(19) United States
(12) Patent Application Publication
(10) Pub. No.: US 2024/0409557 A1
(43) Pub. Date: Dec. 12, 2024

(54) 5,6-FUSED AND 6,6-FUSED BICYCLIC ALCOHOLS AND ETHERS AND COMPOSITIONS FOR USE AS 15-PROSTAGLANDIN DEHYDROGENASE MODULATORS

(71) Applicant: AMGEN INC., Thousand Oaks, CA (US)

(72) Inventors: Kevin Lloyd GREENMAN, Thousand Oaks, CA (US); Alexander J. Pickrell, Westlake Village, CA (US); Kexue LI, Newbury Park, CA (US); Albert K. AMEGADZIE, Westlake Village, CA (US); Hui-Ling WANG, Thousand Oaks, CA (US); Nicholas Anthony WEIRES, Thousand Oaks, CA (US); John G. ALLEN, Newbury Park, CA (US); Matthew P. BOURBEAU, Woodland Hills, CA (US)

(73) Assignee: AMGEN INC., Thousand Oaks, CA (US)

(21) Appl. No.: 18/657,114

(22) Filed: May 7, 2024

Related U.S. Application Data
(60) Provisional application No. 63/501,059, filed on May 9, 2023.
(60) Provisional application No. 63/640,585, filed on Apr. 30, 2024.

(51) Int. Cl. C07D 519/00; A61K 31/437; A61K 31/519; A61P 1/04

(52) U.S. Cl. CPC C07D 519/00; A61K 31/437; A61K 31/519; A61P 1/04

(57) ABSTRACT

The present disclosure provides novel 15-hydroxy-prostaglandin dehydrogenase inhibitors, pharmaceutical compositions comprising such compounds, and methods of using such compounds and compositions in treating 15-hydroxy-prostaglandin dehydrogenase-mediated disease. Also provided are methods of making such compounds and intermediates thereof. The compounds have a general Formula (A).

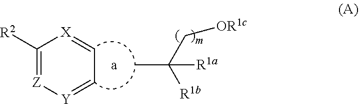

Further, the disclosure provides intermediates useful in the synthesis of compounds of Formula (A).

### CROSS-REFERENCE TO RELATED APPLICATIONS

This application claims the benefit of priority to U.S. Provisional Patent Application No. 63/501,059, filed May 9, 2023, and U.S. Provisional Patent Application No. 63/640,585, filed Apr. 30, 2024, wherein each of the foregoing applications is incorporated herein by reference in its entirety.

### FIELD

Provided herein are novel compounds, pharmaceutical compositions comprising such compounds, and methods of using such compounds and compositions in treating 15-hydroxy-prostaglandin dehydrogenase-mediated disease. Also provided are methods of making such compounds and intermediates thereof.

### BACKGROUND

Short-chain dehydrogenases (SCDs) are a family of dehydrogenases that are involved in synthesis and degradation of fatty acids, steroids, and some prostaglandins. They are therefore implicated in a variety of disorders such as lipid storage disease, myopathy, SCD deficiency, and certain genetic disorders. The SCD 15-hydroxy-prostaglandin dehydrogenase (15-PGDH) (also identified as 15-prostaglandin dehydrogenase or hydroxyprostaglandin dehydrogenase 15-(nicotinamide adeninedinucleotide)), represents a key enzyme in the inactivation of a number of active prostaglandins, leukotrienes and hydroxyeicosatetraenoic acids (HETEs). Recent studies suggest that inhibitors of 15-PGDH and activators of 15-PGDH could be therapeutically valuable. 15-PGDH is responsible for the inactivation of prostaglandin E2 (PGE2), which is a downstream product of cyclooxygenase-2 (COX-2) metabolism. PGE2 has been shown to be beneficial in a variety of biological processes, such as hair density, dermal wound healing, and bone formation.

### SUMMARY

Provided herein are compounds of Formula (A):

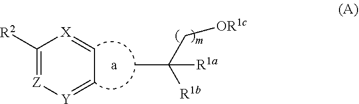

or a pharmaceutically acceptable salt of said compound;
wherein

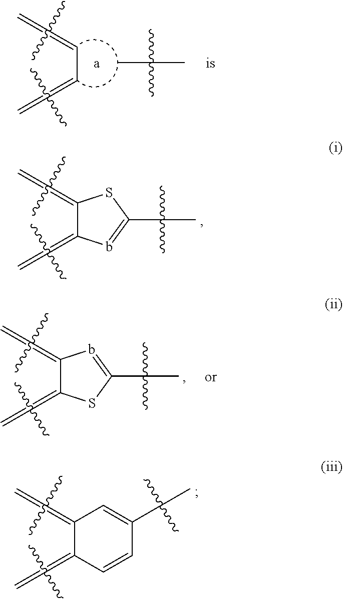

m is 0 or 1;
X is N or CH;
Y is N or C-R³;
Z is N or C-R⁵;
b is N or C-R⁴;
R^{1a} is —H, deuterium, C₁₋₆ alkyl, or C₁₋₆ haloalkyl;
   wherein, when R^{1a} is C₁₋₆ alkyl or C₁₋₆ haloalkyl, then R^{1a} may be unsubstituted or substituted with one or more substituents, each of which independently is halogen, —OH, —N(R^{b})₂, C₁₋₃ alkyl, or C₁₋₃ alkoxy;
R^{1b} is —H, C₁₋₆ alkyl, C₁₋₆ haloalkyl, C₁₋₆ heteroalkyl, —(C₁₋₆ alkylene)-O—(C₁₋₆ alkyl), C₃₋₁₀ cycloalkyl, or heterocyclyl having 3 to 10 ring members;
   wherein, when R^{1b} is C₁₋₆ alkyl, C₁₋₆ haloalkyl, C₁₋₆ heteroalkyl, —(C₁₋₆ alkylene)-O-(C₁₋₆ alkyl), C₃₋₁₀ cycloalkyl, or heterocyclyl having 3 to 10 ring members, then R^{1b} may be unsubstituted or substituted with one or more substituents, each of which independently is halogen, —OH, —N(R^{b})₂, C₁₋₃ alkyl, or C₁₋₃ alkoxy; wherein each of the C₁₋₃ alkyl and C₁₋₃ alkoxy may be unsubstituted or substituted with 1 to 6—F groups;
or, alternatively, R^{1a} and R^{1b} together form a C₃₋₁₀ cycloalkyl, C₃₋₁₀ cycloalkenyl, or heterocyclyl having 3 to 10 ring members;
   wherein the R^{1a} and R^{1b} cycle may be unsubstituted or substituted with one or more substituents, each of which independently is halogen, —OH, oxo, —N(R^{b})₂, C₁₋₃ alkyl, C₁₋₃alkoxy, -CN, C₁₋₆ heteroalkyl, C₃₋₁₀ cycloalkyl, or heterocyclyl having 3 to 10 ring members; wherein each of the C₁₋₃ alkyl and C₁₋₃ alkoxy may be unsubstituted or substituted with 1 to 9 halogen groups or C₁₋₃ alkoxy;
R^{1c} is —H, C₁₋₆ alkyl, or C₃₋₆ cycloalkyl;
   wherein, when R^{1c} is C₁₋₆ alkyl or C₃₋₆ cycloalkyl, then R^{1c} may be unsubstituted or substituted with one or more substituents, each of which independently is halogen, —OH, —N(R^{b})₂, C₁₋₃ alkyl, C₁₋₃ alkoxy, or C₁₋₆heteroalkyl;
R² is C₃₋₂₀ cycloalkyl, C₃₋₂₀ cycloalkenyl, C₆₋₁₈ aryl, heteroaryl having 5 to 20 ring members, or heterocyclyl having 3 to 20 ring members;
   wherein R² may be unsubstituted or substituted with one or more substituents, each of which independently is halogen, —OH, oxo, —N(R^{b})₂, C₁₋₆ alkyl, C₁₋₆haloalkyl, C₁₋₆ alkoxy, C₁₋₆ alkylene-OH, C₁₋₆alkylene-O—C₁₋₆alkyl, or C₁₋₆heteroalkyl;
R³ is —H, C₁₋₆ alkyl, C₁₋₆ haloalkyl, C₃₋₆ cycloalkyl, amino, C₁₋₄alkyl-amino, di-C₁₋₄alkyl-amino, C₆₋₁₀ aryl, heteroaryl having 5 to 10 ring members, or heterocyclyl having 5 to 10 ring members;
   wherein, when R³ is C₁₋₆ alkyl, C₁₋₆haloalkyl, C₃₋₆ cycloalkyl, C₁₋₄alkyl-amino, di-C₁₋₄ alkyl-amino, C₆₋₁₀ aryl, heteroaryl having 5 to 10 ring members, or heterocyclyl having 5 to 10 ring members, then R³ may be unsubstituted or substituted with one or more substituents, each of which independently is halogen, —OH, oxo, —N(R^{b})₂, C₁₋₃ alkyl, C₁₋₃ haloalkyl, C₁₋₃ alkoxy, C₁₋₃ alkylene-C₁₋₃ alkoxy, C₁₋₆ heteroalkyl, or two R³ substituents together form a C₃₋₆ carbocyclyl or a heterocyclyl having 3 to 6 ring members;
R⁴ is —H, C₁₋₃ alkyl, C₁₋₃ haloalkyl, —CN, halogen, or —N(R^{a})₂ where each instance of R^{a}, where present, independently is —H, C₁₋₆ alkyl, or —C(O)C₁₋₆alkyl; or, alternatively, two R^{a} substituents together form a heterocyclyl having 3 to 6 ring members;
   wherein R^{a} may be unsubstituted or substituted with one or more substituents, each of which independently is —F, —Cl, —OH, C₁₋₃ alkoxy; R⁵ is —H, C₁₋₆ alkyl, C₃₋₆ cycloalkyl, C₁₋₆ haloalkyl, or C₁₋₆heteroalkyl;
   wherein, when R is C₁₋₆alkyl, C₃₋₆ cycloalkyl, C₁₋₆ haloalkyl, or C₁₋₆ heteroalkyl, then R may be unsubstituted or substituted one or more substituents, each of which independently is halogen, —OH, C₁₋₃ alkyl, or C₁₋₃ alkoxy;
where each instance of R^{b}, where present, independently is —H or C₁₋₆alkyl; or, alternatively, two R^{b} substituents together form a heterocyclyl having 3 to 6 ring members; and
   wherein, when R^{b} is C₁₋₆ alkyl or, alternatively, two R^{b} substituents together form a heterocyclyl having 3 to 6 ring members, then R^{b} may be unsubstituted or substituted with one or more substituents, each of which independently is —F, —Cl, —OH, C₁₋₃ alkoxy.

In several embodiments, R^{1a} is —H, C₁₋₆ alkyl, C₁₋₆ haloalkyl, or C₁₋₆ heteroalkyl. In several embodiments, R^{1a} may be unsubstituted or substituted with 1, 2, 3, or 4 substituents, each of which independently is halogen, —OH, alkyl, or alkoxy.

In several embodiments, R^{1b} is —H, C₁₋₆ alkyl, C₁₋₆ haloalkyl, C₃₋₁₀ cycloalkyl, or heterocyclyl having 3 to 10 ring members. In several embodiments, R^{1b} may be unsubstituted or substituted 1, 2, 3, or 4 substituents, each of which independently is halogen, —OH, alkyl, or alkoxy.

In several embodiments, R^{1a} and R^{1b} together form a C₃₋₁₀ cycloalkyl, C₃₋₁₀ cycloalkenyl, or heterocyclyl having 3 to 10 ring members. In several embodiments, where R^{1a} and R^{1b} together provide a cycle, the R^{a} and R^{1b} cycle may be unsubstituted or substituted with 1, 2, 3, or 4 substituents, each of which independently is halogen, —OH, alkyl, oxo, alkoxy, or heteroalkyl.

In several embodiments, R^{1c} is —H, C₁₋₆ alkyl, or C₃₋₆ cycloalkyl. In several embodiments, R^{1c} may be unsubstituted or substituted 1, 2, 3, or 4 substituents, each of which independently is halogen, —OH, alkyl, alkoxy, or heteroalkyl.

In several embodiments, R² is C₃₋₁₀ cycloalkyl, C₃₋₁₀ cycloalkenyl, C₈₋₁₀ cycloalkynyl, C₆₋₁₀ aryl, heteroaryl having 5 to 10 ring members, or heterocyclyl having 3 to 10 ring members. In several embodiments, R² may be unsubstituted or substituted with 1, 2, 3, 4, or 5 substituents, each of which independently is halogen, —OH, oxo, alkyl, haloalkyl, alkoxy, alkylene-OH, alkylene-O-alkyl, or heteroalkyl.

In several embodiments, R³ is —H, C₁₋₆ alkyl, C₁₋₆ haloalkyl, C₃₋₆ cycloalkyl, amino, C₁₋₄alkyl-amino, di-C₁₋₄alkyl-amino, C₆₋₁₀ aryl, heteroaryl having 5 to 10 ring members, or heterocyclyl having 5 to 10 ring members. In several embodiments, R³ may be unsubstituted or substituted with 1, 2, 3, 4, or 5 substituents, each of which independently is halogen, —OH, oxo, alkyl, haloalkyl, alkoxy, alkylene-alkoxy, heteroalkyl, or two R³ substituents together form a carbocyclyl or a heterocyclyl.

In several embodiments, R⁴ is —H, C₁₋₃ alkyl, or —N(R^{a})₂. In several embodiments, R^{a}, where present, independently is —H, C₁₋₃ alkyl, or C₁₋₆heteroalkyl. In several embodiments, R^{a}, where present, independently is —H or C₁₋₃ alkyl. In several embodiments, R^{a} may be unsubstituted or substituted with 1, 2, 3, 4, or 5 substituents, each of which independently is halogen, —OH, C₁₋₃ haloalkyl, C₁₋₃ alkoxy, or two R^{a} substituents together form a C₃₋₆ carbocyclyl or a heterocyclyl having 3 to 6 ring members.

In several embodiments, R⁵ is —H, C₁₋₆ alkyl, C₃₋₆ cycloalkyl, C₁₋₆ haloalkyl, or C₁₋₆heteroalkyl.

In several embodiments, R² may be unsubstituted or substituted 1, 2, 3, or 4 substituents, each of which independently is halogen, —OH, alkyl, or alkoxy.

In several embodiments, when R² is unsubstituted cyclopropyl, unsubstituted phenyl, or unsubstituted or substituted cycloalkenyl, then one of R^{1a}, R^{1b}b, and R^{1c} is not —H.

Several embodiments pertain to a pharmaceutical composition comprising a compound or salt of Formula (A) (which may be further defined as a compound or salt of Formula (I)) and a pharmaceutically acceptable excipient.

Several embodiments pertain to a compound or salt of Formula (A) (e.g., including a compound or salt of Formula (I)) or a pharmaceutical composition comprising a compound of Formula (A) for use as a medicament.

Several embodiments pertain to a compound or salt of Formula (A) (which may be further defined as a compound or salt of Formula (I)) or a pharmaceutical composition comprising a compound of Formula (A) for use in treating a 15-PGDH mediated disease or disorder.

Several embodiments pertain to a Formula (A) (e.g., including a compound or salt of Formula (I)) or a pharmaceutical composition comprising a compound of Formula (A) for use in treating inflammatory bowel disease.

Several embodiments pertain to a compound or salt of Formula (A) (which may be further defined as a compound or salt of Formula (I)) or a pharmaceutical composition comprising a compound of Formula (A) for use in treating ulcerative colitis.

Several embodiments pertain to a compound or salt of Formula (A) (which may be further defined as a compound or salt of Formula (I)) or a pharmaceutical composition comprising a compound of Formula (A) for use in treating Crohn's disease.

Several embodiments pertain to a compound or salt of Formula (A) (which may be further defined as a compound or salt of Formula (I)) or a pharmaceutical composition comprising a compound of Formula (A) for use in treating a fibrotic disease, disorder or condition.

Several embodiments pertain to a method of treating a 15-PGDH mediated disease in a subject in need thereof, the method comprising administering to the subject therapeutically an effective amount of a compound or salt of Formula (A) (which may be further defined as a compound or salt of Formula (I)) or a pharmaceutical composition comprising a compound of Formula (A).

Several embodiments pertain to a method of treating intestinal, gastrointestinal, or bowel disorders in a subject in need thereof, the method comprising administering to the subject therapeutically an effective amount of a compound or salt of Formula (A) (which may be further defined as a compound or salt of Formula (I)) or a pharmaceutical composition comprising a compound of Formula (A).

Further aspects and advantages will be apparent to those of ordinary skill in the art from a review of the following detailed description. The description hereafter includes specific cases, embodiments, and examples with the understanding that the disclosure is illustrative and is not intended to limit the embodiments of the present disclosure to the specific cases, embodiments, and examples described herein. To the contrary, reference to embodiments of the present disclosure is intended to cover alternatives, modifications, and equivalents as may be included within the spirit and scope of the embodiments of the present disclosure as defined by the appended claims.

### BRIEF DESCRIPTION OF THE DRAWINGS

FIGS. 1A-1D provide colon length data for mice having dextran sodium sulfate (DSS) induced colitis and treated with one of four different compounds of Formula (A). FIG. 1A shows data for mice treated with Compound B15S versus mice treated with vehicle only or naïve mice. FIG. 1B shows data for mice treated with Compound B15R versus mice treated with vehicle only or naïve mice.

FIG. 1C shows data for mice treated with Compound B3OR versus mice treated with vehicle only or naïve mice. FIG. 1D shows data for mice treated with Compound B41R versus mice treated with vehicle only or naïve mice.

FIGS. 2A-2D provide histopathological data for mice having DSS induced colitis and treated Compound B15R versus mice receiving vehicle only or naïve mice. FIG. 2A provides the sum of histopathology scores. FIG. 2B provides inflammation scores. FIG. 2C provides erosion scores.

FIG. 2D provides gland loss scores.

FIGS. 3A-3D provide histopathological data for mice having DSS induced colitis and treated Compound B30R versus mice receiving vehicle only or naïve mice. FIG. 3A provides the sum of histopathology scores. FIG. 3B provides inflammation scores. FIG. 3C provides erosion scores.

FIG. 3D provides gland loss scores.

FIGS. 4A-4D provide histopathological data for mice having DSS induced colitis and treated Compound B41R versus mice receiving vehicle only or naïve mice. FIG. 4A provides the sum of histopathology scores. FIG. 4B provides inflammation scores. FIG. 4C provides erosion scores.

FIG. 4D provides gland loss scores.

### DETAILED DESCRIPTION

Several embodiments provide compounds and compositions, methods of use thereof, and methods of manufacture thereof. In several embodiments, the compounds disclosed herein are useful for inhibiting SCD and/or 15-PGDH and/or for treating SCD and/or 15-PGDH diseases, disorders, and conditions. In several embodiments, the compounds disclosed herein have a structure of Formula (A). In several embodiments, the compounds disclosed herein have a structure of Formula (I). In several embodiments, the compounds disclosed herein have either a benzothiophenyl or a 5,6-fused thieno-heterocyclyl (e.g., thieno-pyridinyl) core structure. In several embodiments, the compound comprises an alcohol or ether moiety located on a carbon alpha to the 2-position of a benzothiophenyl core or 5,6-fused thieno-heterocyclyl (e.g., thieno-pyridinyl) core. In several embodiments, the compound comprises an alcohol located on a carbon alpha to the 2-position of a benzothiophenyl core or 5,6-fused thieno-heterocyclyl (e.g., thieno-pyridinyl) core. Elsewhere herein, these compounds may be referred to as benzothiophenyl alcohols or 5,6-fused thieno-heterocyclyl alcohols (e.g., thieno-pyridinyl alcohols) for brevity. These compounds and any other compounds disclosed herein may be generally referred to as 15-PGDH inhibitors.

The following description provides context and examples, but should not be interpreted to limit the scope of the inventions covered by the claims that follow in this specification or in any other application that claims priority to this specification. No single component or collection of components is essential or indispensable. The section headings used herein are for organizational purposes only and are not to be construed as limiting the subject matter described. Features disclosed under one heading (such as a composition or combination) can be combined with features disclosed under a different heading (a method of treating).

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

As used herein, “Cₐ-b” (or similar wording, such as, Cₐ to C_{b}) in which “a” and “b” are integers refer to the number of carbon atoms in an alkyl, alkylene, alkenyl, alkenylene, alkynyl, alkynylene, haloalkyl, alkoxy, thioalkyl, heteroalkyl, cycloalkyl, cycloalkenyl, cycloalkynyl, aryl, heteroaryl, or other group. That is, the alkyl, alkenyl, alkynyl, ring of the cycloalkyl, ring of the cycloalkenyl, ring of the cycloalkynyl, ring of the aryl, or the ring of the heteroaryl can contain from “a” to “b”, inclusive, carbon atoms. Thus, for example, a “C₁₋₄ alkyl” group (or C₁ to C₄ alkyl) refers to all alkyl groups having from 1 to 4 carbons (e.g., 1, 2, 3, or 4), that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. A “C₁₋₆ alkyl” group refers to all alkyl groups having from 1 to 6 carbons (e.g., 1, 2, 3, 4, 5, or 6). If no “a” and “b” are designated with regard to a group, the ranges described in these definitions are envisioned. As will be appreciated in view of the preceding disclosure, where a range is disclosed (for alkyl groups or other groups disclosed herein), each individual member within that disclosed range is also envisioned and disclosed. Thus, where a “C₁₋₆ alkyl” is disclosed, all alkyl groups having from 1 to 6 carbons and any alkyl groups containing 1 carbon, 2 carbons, 3 carbons, 4 carbons, 5 carbons, or 6 carbons, or combinations of the foregoing (e.g., 1, 2, 3, or 5 carbons; 2, 3, 4, or 6 carbons; etc.) are provided. Likewise, where a “C₁₋₃ alkyl” is disclosed, all alkyl groups having from 1 to 3 carbons and any alkyl groups containing 1 carbon, 2 carbons, or 3 carbons, or combinations of the foregoing (e.g., 1 or 3; 1 or 2; 2 or 3; etc.) are provided.

The term “alkyl” refers to a straight or branched hydrocarbon chain that is fully saturated (i.e., contains no double or triple bonds). Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 12 carbon atoms. The “alkyl” group could also be a lower alkyl having 1 to 6 carbon atoms. A C₁₋₅ alkyl includes C₅ alkyls, C₄ alkyls, C₃ alkyls, C₂ alkyls and C₁ alkyl (i.e., methyl). A C₁₋₆ alkyl includes all moieties described above for C₁₋₅ alkyls but also includes C₆ alkyls. A C₁₋₁₀ alkyl includes all moieties described above for C₁₋₅ alkyls and C₁₋₆ alkyls, but also includes C₇, C₈, C₉ and C₁₀ alkyls. Similarly, a C₁₋₁₂ alkyl includes all the foregoing moieties, but also includes C₁₁ and C₁₂ alkyls. By way of example only, “C₁₋₄ alkyl” indicates that there are one to four carbon atoms in the alkyl chain, i.e., the alkyl chain is methyl, ethyl, propyl, iso-propyl, n-butyl, iso-butyl, sec-butyl, or t-butyl. Non-limiting examples of C₁₋₁₂ alkyl include, but are in no way limited to, methyl (“Me” or —CH₃), ethyl, n-propyl, i-propyl, sec-propyl, n-butyl, i-butyl, sec-butyl, t-butyl, n-pentyl, neo-pentyl, n-hexyl, n-heptyl, n-octyl, n-nonyl, n-decyl, n-undecyl, and n-dodecyl.

The term “alkylene” or “alkylene chain” refers to a fully saturated, straight or branched divalent hydrocarbon chain radical, and having from one to twelve carbon atoms. Examples of alkylene groups include, but are not limited to, methylene, ethylene, propylene, butylene, pentylene, hexylene, heptylene and octylene. A C₁₋₁₂ alkylene includes C₁₂ alkylenes, C₁₁ alkylenes, C₁₀ alkylenes, C₉ alkylenes, Cs alkylenes, C₇ alkylenes, C₆ alkylenes, C₅ alkylenes, C₄ alkylenes, C₃ alkylenes, and C₂ alkylenes, and C₁ alkylene (i.e., methylene). An alkylene group may be a lower alkylene having 1 to 6 carbon atoms. A lower alkylene includes C₆ alkylenes, C₅ alkylenes, C₄ alkylenes, C₃ alkylenes, and C₂ alkylenes, and C₁ alkylene. It also is to be understood that certain diradical naming conventions can include either mono-radical or di-radical naming conventions, depending on the context. For example, where a position of a substituent within a molecule requires two points of attachment to the rest of the molecule, it is understood that the substituent is a di-radical. For example, a substituent identified as alkyl but that requires two points of attachment includes alkylene di-radicals such as —CH₂—, —CH₂CH₂—, —CH₂CH(CH₃)CH₂—, and the like.

The term “alkenyl” or “alkenyl group” refers to a straight or branched hydrocarbon chain radical having from two to twelve carbon atoms and having one or more carbon-carbon double bonds. An alkenyl group comprising up to 12 carbon atoms is a C₂₋₁₂ alkenyl, an alkenyl comprising up to 10 carbon atoms is a C₂₋₁₀ alkenyl, an alkenyl group comprising up to 6 carbon atoms is a C₂₋₆ alkenyl and an alkenyl comprising up to 5 carbon atoms is a C₂₋₅ alkenyl. A C₂₋₅ alkenyl includes C₅ alkenyls, C₄ alkenyls, C₃ alkenyls, and C₂ alkenyls. A C₂₋₆ alkenyl includes all moieties described above for C₂₋₅ alkenyls but also includes C₆ alkenyls. A C₂₋₁₀ alkenyl includes all moieties described above for C₂₋₅ alkenyls and C₂₋₆ alkenyls, but also includes C₇, C₈, C₉ and C₁₀ alkenyls. A C₂₋₁₂ alkenyl includes all the foregoing moieties, but also includes C₁₁ and C₁₂ alkenyls. Non-limiting examples of C₂₋₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl.

The term “alkynyl” or “alkynyl group” refers to a straight or branched hydrocarbon chain radical having from two to twelve carbon atoms and having one or more carbon-carbon triple bonds. An alkynyl group comprising up to 12 carbon atoms is a C₂₋₁₂ alkynyl, an alkynyl comprising up to 10 carbon atoms is a C₂₋₁₀ alkynyl, an alkynyl group comprising up to 6 carbon atoms is a C₂₋₆ alkynyl and an alkynyl comprising up to 5 carbon atoms is a C₂₋₅ alkynyl. A C₂₋₅ alkynyl includes C₅ alkynyls, C₄ alkynyls, C₃ alkynyls, and C₂ alkynyls. A C₂₋₆ alkynyl includes all moieties described above for C₂-s alkynyls but also includes C₆ alkynyls. A C₂₋₁₀ alkynyl includes all moieties described above for C₂-s alkynyls and C₂₋₆ alkynyls, but also includes C₇, C₈, C₉ and C₁₀ alkynyls. A C₂₋₁₂ alkynyl includes all the foregoing moieties, but also includes C₁₁ and C₁₂ alkynyls. Non-limiting examples of C₂₋₁₂ alkynyl include ethynyl, propynyl, butynyl, pentynyl and the like.

The term “halogen” or “halo,” as used herein, means any one of the radio-stable atoms of column 7 of the Periodic Table of the Elements, e.g., fluorine (—F), chlorine (—Cl), bromine (—Br), or iodine (—I).

The term “haloalkyl” refers to a straight- or branched-chain alkyl group, substituting one or more or all hydrogens (e.g., —H) with halogens. Examples of haloalkyl groups include, but are not limited to, —CF₃, —CHF₂, —CH₂F, —CH₂CF₃, —CH₂CHF₂, —CH₂CH₂F, —CH₂CH₂Cl, —CH₂CF₂CF₃ and other groups that in light of the ordinary skill in the art and the teachings provided herein, would be considered equivalent to any one of the foregoing examples. The haloalkyl may be a lower haloalkyl. The haloalkyl may be perhalogenated (e.g., perfluorinated).

The term “alkoxy” refers to the formula —OR wherein R is an alkyl group as defined elsewhere herein. For illustration, a “C₁₋₉ alkoxy” includes but is not limited to methoxy, ethoxy, n-propoxy, 1-methylethoxy (isopropoxy), n-butoxy, iso-butoxy, sec-butoxy, tert-butoxy, and the like.

The term “heteroalkyl” refers to a straight or branched hydrocarbon chain (e.g., alkyl) containing one or more heteroatoms each of which independently is nitrogen (e.g., amino, etc.), oxygen (e.g., alkoxy, ether, hydroxyl, etc.), or sulfur (e.g., thiol, sulfide, etc.). The heteroalkyl group may have 1 to 12 carbon atoms although the present definition also covers the occurrence of the term “heteroalkyl” where no numerical range is designated. The heteroalkyl group could also be a lower heteroalkyl having 1 to 6 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain. As will be understood from the above, in embodiments having one heteroatom, the heteroatom may be found anywhere along the alkyl portion of the heteroalkyl group, including in the first position (e.g., the heteroatom of the heteroalkyl may serve as the atom that directly attaches the alkyl to the rest of the molecule).

The term “aryl” refers to a hydrocarbon ring system radical comprising hydrogen and at least one aromatic ring. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. For example, the aryl group can be a C₆₋₁₄ aryl group, a C₆₋₁₀ aryl group, or a C₆ aryl group. For purposes of this invention, the aryl radical can be a monocyclic, bicyclic, tricyclic or tetracyclic ring system, which can include fused or bridged ring systems, wherein at least one ring in the system is aromatic. Examples of aryl groups include, but are not limited to, phenyl, aceanthrylenyl, acenaphthylenyl, acephenanthrylenyl, anthracenyl, azulenyl, chrysenyl, fluoranthenyl, fluorenyl, as-indacene-yl, s-indacene-yl, indanyl, indenyl, naphthalenyl, phenalenyl, phenanthrenyl, pleiadenyl, pyrenyl, and triphenylenyl.

The term “carbocyclyl” means a non-aromatic cyclic ring or ring system containing only carbon atoms in the ring system backbone and comprising 3 to 20 ring members (3 to 20 carbon atom ring members; C₃₋₂₀). When the carbocyclyl is a ring system, two or more rings may be joined together in a fused, bridged or spiro-connected fashion. Carbocyclyls include cycloalkyls, cycloalkenyls, and cycloalkynyls. The carbocyclyl group may be a medium size carbocyclyl having 3 to 10 carbon atoms.

The carbocyclyl group could have 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

The term “cycloalkyl” refers to a non-aromatic monocyclic or polycyclic fully saturated hydrocarbon radical consisting solely of carbon and hydrogen atoms, which can include fused, bridged, or spiro ring systems, having from 3 to 20 carbon atom ring members (e.g., having from 3 to 10 ring atoms, 3 to 8 ring atoms, etc.), and which is attached to the rest of the molecule by a single bond. No ring in a cycloalkyl ring or ring system is aromatic. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like.

The term “cycloalkenyl” refers to a monocyclic or polycyclic hydrocarbon radical consisting solely of carbon and hydrogen atoms, having one or more carbon-carbon double bonds, which can include fused, bridged, or spiro ring systems, having from 3 to 20 carbon ring members (e.g., having from 3 to 10 ring atoms, 4 to 10 ring atoms, etc.) and which is attached to the rest of the molecule by a single bond. No ring in a cycloalkenyl ring or ring system is aromatic. An example is cyclohexenyl, cycloalkenyl groups can contain 4 to 10 atoms in the ring(s). Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like.

The term “cycloalkynyl” refers to a monocyclic or polycyclic hydrocarbon radical consisting solely of carbon and hydrogen atoms, having one or more carbon-carbon triple bonds, which can include fused, bridged, or spiro ring systems, having from 8 to 20 carbon ring members (e.g., having from 8 to 10 ring atoms, etc.) and which is attached to the rest of the molecule by a single bond. No ring in a cycloalkynyl ring or ring system is aromatic. Monocyclic cycloalkynyl radicals include, for example, cycloheptynyl, cyclooctynyl, and the like.

The term “heterocyclyl” refers to three-, four-, five-, six-, seven-, eight-, nine-, ten-, up to 20-membered monocyclic, bicyclic, and tricyclic ring systems wherein carbon atoms together with from 1 to 5 heteroatoms (each of which independently is nitrogen (e.g., N, NH, N-R, etc.), oxygen, or sulfur (e.g., S, S(O), S(O)₂)) constitute said ring system. Heterocyclyl or heterocyclic rings include non-heteroaryl ring systems (e.g., those cycles comprising heteroatoms as ring members that do not fall within the definition of a “heteroaryl”). Unless stated otherwise specifically in the specification, the heterocyclyl radical can be a monocyclic, bicyclic, tricyclic or tetracyclic ring system, which can include fused, bridged, and spiro ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, azetidinyl, aziridinyl, 1,3-dioxin-yl, 1,3-dioxanyl, 1,4-dioxanyl, 1,3-oxathianyl, 1,3-oxathiolanyl, 1,3-dithiolyl, 1,3-dithiolanyl, 1,4-oxathianyl, tetrahydro-1,4-thiazinyl, dioxolanyl, decahydroisoquinolyl, imidazolidinyl, isothiazolidinyl, isoxazolidinyl, maleimidyl, morpholinyl, octahydroindolyl, octahydroisoindolyl, oxetanyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, succinimidyl, thiazolidinyl, tetrahydrofuranyl, trithianyl, tetrahydropyranyl, thiomorpholinyl, thiamorpholinyl, 1-oxo-thiomorpholinyl, 1,1-dioxo-thiomorpholinyl, and the like. The point of attachment of the heterocyclyl, heterocyclic ring, or heterocycle to the rest of the molecule by a single bond is through a ring member atom, which can be carbon or nitrogen. A heterocycle may optionally contain one or more unsaturated bonds situated in such a way, however, that a fully delocalized pi-electron system does not occur in any ring of the ring system. The heterocyclyl group may be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations (e.g., a heterocyclyl having 3 to 6 ring members or a 3- to 6-membered heterocyclyl).

The term “heteroaryl” refers to a 5- to 20-membered ring system radical with 1 to 19 carbon atoms and 1 to 6 heteroatoms (each of which independently is nitrogen (e.g., N, NH, N-R, etc.), oxygen, or sulfur) as the ring members. The heteroaryl radical can be a monocyclic, bicyclic, tricyclic or tetracyclic ring system, which can include fused or bridged ring systems, wherein at least one ring is aromatic. The nitrogen, carbon or sulfur atoms in the heteroaryl radical can be optionally oxidized and the nitrogen atom can be optionally quaternized. The heteroaryl group can contain 5 to 14 ring members (atoms in the ring(s)), 5 to 10 ring members (atoms in the ring(s)), 5 to 9 ring members (atoms in the ring(s)), 5 to 7 ring members (atoms in the ring(s)), 5 to 6 ring members (atoms in the ring(s)). The heteroaryl group may be a medium size heteroaryl having 5 to 10 ring members. The heteroaryl group could also be a heteroaryl having 5 to 6 ring members. The heteroaryl group could also be a heteroaryl having 6 to 9 ring members. The heteroaryl group could also have 6 ring members. The heteroaryl group could also have 9 ring members. In various embodiments, a heteroaryl contains from 1 to 6 heteroatoms, from 1 to 5 heteroatoms, from 1 to 4 heteroatoms, from 1 to 3 heteroatoms, from 1 to 2 heteroatoms, or 1 heteroatom. For example, in various embodiments, a heteroaryl contains 1 to 5 nitrogen atoms, 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, 1 sulfur or oxygen atom, etc. Examples include, but are not limited to, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophene-yl), benzotriazolyl, benzo[4,6]imidazo[1,2-a]pyridinyl, carbazolyl, cinnolinyl, dibenzofuranyl, dibenzothiophene-yl, furanyl, isothiazolyl, imidazolyl, imidazo[1,2-a]pyridinyl, indazolyl, indolyl, indazolyl, isoindolyl, indolinyl, isoindolinyl, isoquinolyl, indolizinyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrazolopyridinyl, pyridine-onyl, quinazolinyl, quinoxalinyl, quinolinyl, isoquinolinyl, tetrahydroquinolinyl, thiazolyl, thiadiazolyl, triazolyl, triazolopyridinyl, tetrazolyl, triazinyl, and thiophene-yl (i.e., thienyl).

The term “hydroxy” (or “hydroxyl”) refers to a —OH group.

The term “cyano” refers to a “—CN” group.

The term “oxo” refers to the =0 substituent.

The term “amino” refers to a NH₂ group.

The term “alkyl-amino” refers to a “—NR_{A}H” group in which R_{A} is alkyl.

The term “di-alkyl-amino” refers to a “—NR_{A}R_{B}” group in which R_{A} and R_{B} are each independently alkyl. R_{A} and R_{B} may be taken together with the nitrogen to which they are attached to provide a heteroaryl or heterocyclyl.

As used herein, any “R” group(s) such as, without limitation, R¹, R², R³, etc., represent substituents that can be attached to the indicated atom. An R group may be unsubstituted or substituted. If two “R” groups are described as being “taken together” (or similar language), the R groups and the atoms they are attached to can form a cycle (e.g., cycloalkyl, aryl, heteroaryl, heterocyclyl). When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

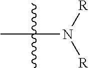

and each instance of R is defined as being independently hydrogen or alkyl, or each instance of R together with the nitrogen to which they are attached form a heterocyclyl, it is meant that each instance of R can be independently hydrogen or alkyl, or alternatively, the substructure has structure:

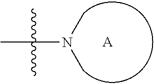

where ring A is a heterocyclyl ring containing the depicted nitrogen. As further illustration, without limitation, if R_{A} and R_{B} of an NR_{A}R_{B} group are indicated to be “taken together,” it means that they are covalently bonded to one another to form a ring:

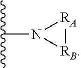

A cyclic structure may be shown using provided using the following structure (or a similar structure with a different ring, heteroatoms, unsaturated bonds, etc.):

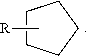

When a cyclic structure is depicted using this type of illustration, what is meant is that the R group may be attached to any position of the ring by replacing an -H of the ring with -R. For example, for the following ring:

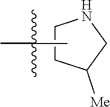

it is meant to include any of the following structures:

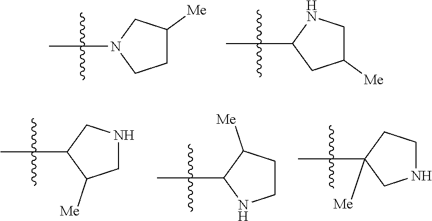 [SMILES: *N1CCC(C)C1 ; *C1CC(C)CN1 ; *C1CNCC1C ; *C1NCCC1C ; *C1(C)CCNC1]

where “ [symbol] ” indicates a bond to a remaining portion of the structure. Likewise, for the following structure:

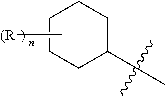

where “ [symbol] ” indicates a bond to a remaining portion of the structure and n is 1 to 6, any of the following structures are envisioned or other variations (as would be readily appreciated by the one of skill in the art):

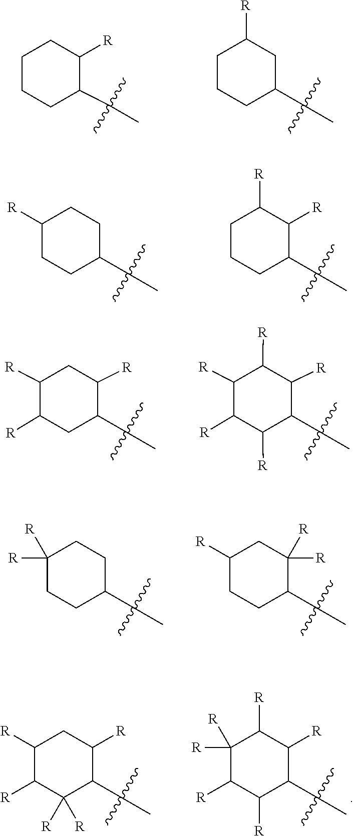

Additionally, where a nitrogen atom is present in the cycle, a hydrogen atom may be removed from the nitrogen to provide a substitution. Thus, for the following structure:

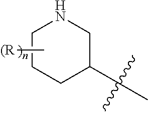

where “ [symbol] ” indicates a bond to a remaining portion of the structure and n is 1 to 6, any of the following structures are envisioned or other variations (as would be readily appreciated by the one of skill in the art):

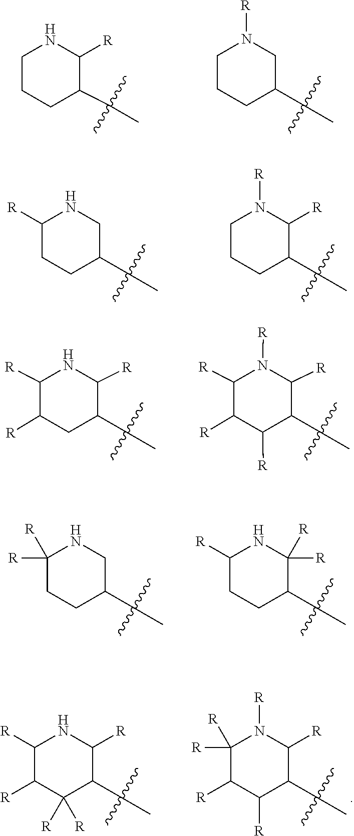

Where specified, a nitrogen atom in the cycle may provide a cation. Thus, for the following structure:

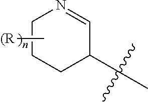

where “ [symbol] ” indicates a bond to a remaining portion of the structure and n is 1 to 6, any of the following structures are envisioned or other variations (as would be readily appreciated by the one of skill in the art):

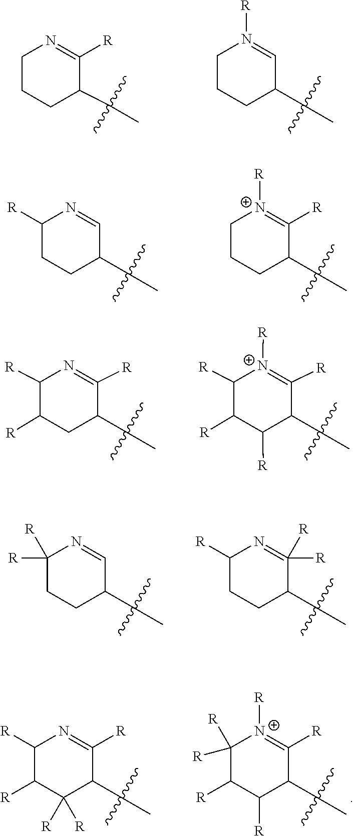

When two “adjacent” R groups are said to form a ring “together with the atoms to which they are attached,” it is meant that the collective unit of the atoms, intervening bonds, and the two R groups are the recited ring. For example, when the following substructure is present:

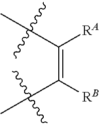

and R^{A} and R^{B} are each independently hydrogen or alkyl, or R¹ and R²together with the atoms to which they are attached form an aryl or carbocyclyl, it is meant that R¹ and R² can be selected from hydrogen or alkyl, or alternatively, the substructure has structure:

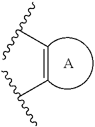

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

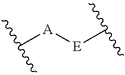

includes the substituent being oriented such that the “A” is attached at the leftmost attachment point of the molecule as well as the case in which “A” is attached at the rightmost attachment point of the molecule.

As noted in the definition for alkylene, it also is to be understood that certain radical naming conventions can include either a mono-radical or a di-radical, depending on the context. For example, where a substituent (e.g., in a genus structure) requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other examples a substituent may require two points of attachment include alkoxy, aryl, heteroaryl, carbocyclyl, heterocyclyl, etc.

As used herein, a radical indicates species with a single, unpaired electron such that the species containing the radical can be covalently bonded to another species. Hence, in this context, a radical is not necessarily a free radical. Rather, a radical indicates a specific portion of a larger molecule.

Where a particular group (e.g., an R group) is indicated as being substituted, it is understood that such substitutions occur where valency allows. Thus, if the group is R and R is —H or alkyl with R is indicated as being unsubstituted or substituted, then it will be understood that the “—H” atom is not substituted because valency does not allow it to be. On the other hand, where R is alkyl, it may be unsubstituted or substituted.

“Solvate” refers to the compound formed by the interaction of a solvent and a compound described herein, a metabolite, or salt thereof. A solvate means solvent addition forms that contain either stoichiometric or non-stoichiometric amounts of solvent. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate. Non-limiting examples of hydrates include monohydrates, dihydrates, etc. Nonlimiting examples of solvates include ethanol solvates, acetone solvates, etc. The scope of the instant disclosure is to be understood to encompass all solvents of the compounds disclosed herein and the stereoisomers, tautomers and isotopically-labelled forms thereof or a pharmaceutically acceptable salt of any of the foregoing.

The term “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be prepared using and/or formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, camphorsulfonic acid, maleic acid, malonic acid, succinic acid, fumaric acid, formic acid, tartaric acid, citric acid, benzoic acid, cinnamic acid, mandelic acid, methanesulfonic acid, ethanesulfonic acid, p-toluenesulfonic acid, salicylic acid, carbonic acid, and the like. Pharmaceutically acceptable base addition salts can be prepared using and/or formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Organic bases from which salts can be derived also include, for example, ethylenediamine, N-methyl-glucamine, lysine, arginine, ornithine, choline, N,N-dibenzylethylenediamine, chloroprocaine, diethanolamine, procaine, N-benzylphenethylamine, piperazine, tris-(hydroxymethyl)-aminomethane, tetramethylammonium hydroxide, dibenzylamine, ephenamine, dehydroabietylamine, N-ethylpiperidine, benzylamine, tetramethylammonium, tetraethylammonium, methylamine, dimethylamine, ethylamine, basic amino acids, and the like. Other salts are known in the art, as described in International Publication No. WO87/05297 (incorporated by reference herein in its entirety). Additionally, the salts of the compounds described herein, can exist in either hydrated or unhydrated (the anhydrous) form or as solvates with other solvent molecules.

As used herein, “intermediate” compounds, includes structures produced from the synthetic procedures described, whether isolated or generated in-situ and not isolated, prior to obtaining the finally desired compound. These intermediates are included in the scope of this disclosure. Exemplary embodiments of such intermediate compounds are set forth elsewhere herein.

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropoisomers. Accordingly, the scope of the instant disclosure is to be understood to encompass all possible stereoisomers of the illustrated compounds, including the stereoisomerically pure form (for example, geometrically pure, enantiomerically pure (e.g., (R), (S), (R)(R), (S)(S), etc.), diastereomerically pure, and atropoisomerically pure) and stereoisomeric mixtures (e.g., both (R) and (S), etc., diastereomers, and atropoisomers, or mixture of any of the foregoing) of any chemical structures disclosed herein (in whole or in part), unless the stereochemistry is specifically identified.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rats and mice but also includes many other species.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements.

When referring to numerical values, the terms “or ranges including and/or spanning the aforementioned values” (and variations thereof) is meant to include any range that includes or spans the aforementioned values. For example, when the temperature of a reaction is expressed as “20° C., 30° C., 40° C., 50° C., or ranges including and/or spanning the aforementioned values,” this includes the particular temperature provided (e.g., 20° C., 30° C., 40° C., or 50° C.) or temperature ranges extending between 20° C. to 50° C., 20° C. to 40° C., 20° C. to 30° C., 30° C. to 50° C., 30° C. to 40° C., or 40° C. to 50° C.

### Compounds

Provided herein as Embodiment 1 is a compound of Formula (A):

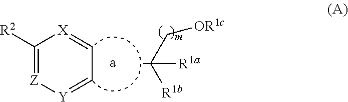

or a pharmaceutically acceptable salt of said compound;

wherein is (i), (ii), or (iii);

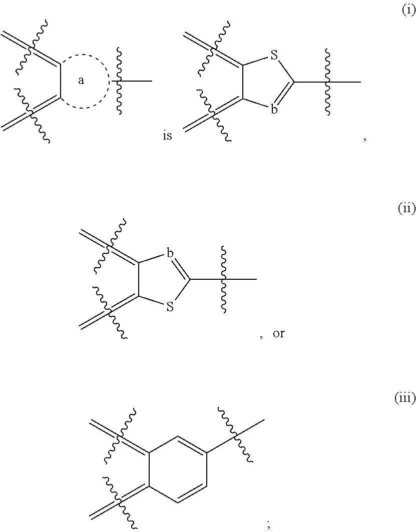

m is 0 or 1;
X is N or CH;
Y is N or C—R³;
Z is N or C—R⁵;
b is N or C—R⁴;
R^{1a} is —H, deuterium, C₁₋₆ alkyl, or C₁₋₆ haloalkyl;
   wherein, when R^{1a} is C₁₋₆ alkyl or C₁₋₆ haloalkyl, then R^{1a} may be unsubstituted or substituted with one or more substituents, each of which independently is halogen, —OH, —N(R^{b})₂, C₁₋₃ alkyl, or C₁₋₃ alkoxy;
R^{1b} is —H, C₁₋₆ alkyl, C₁₋₆ haloalkyl, C₁₋₆ heteroalkyl, —(C₁₋₆ alkylene)-O-(C₁₋₆ alkyl), C₃₋₁₀ cycloalkyl, or heterocyclyl having 3 to 10 ring members;
   wherein, when R^{1b} is C₁₋₆ alkyl, C₁₋₆ haloalkyl, C₁₋₆ heteroalkyl, —(C₁₋₆ alkylene)-O—(C₁₋₆ alkyl), C₃₋₁₀ cycloalkyl, or heterocyclyl having 3 to 10 ring members, then R^{1b} may be unsubstituted or substituted with one or more substituents, each of which independently is halogen, —OH, —N(R^{b})₂, C₁₋₃ alkyl, or C₁₋₃ alkoxy; wherein each of the C₁₋₃ alkyl and C₁₋₃ alkoxy may be unsubstituted or substituted with 1 to 6—F groups;
or, alternatively, R^{1a} and R^{1b} together form a C₃₋₁₀ cycloalkyl, C₃₋₁₀ cycloalkenyl, or heterocyclyl having 3 to 10 ring members;
   wherein the R^{1a} and R^{1b} cycle may be unsubstituted or substituted with one or more substituents, each of which independently is halogen, —OH, oxo, —N(R^{b})₂, C₁₋₃ alkyl, C₁₋₃alkoxy, -CN, C₁₋₆ heteroalkyl, C₃₋₁₀ cycloalkyl, or heterocyclyl having 3 to 10 ring members; wherein each of the C₁₋₃ alkyl and C₁₋₃ alkoxy may be unsubstituted or substituted with 1 to 9 halogen groups or C₁₋₃ alkoxy;
R^{1c} is —H, C₁₋₆ alkyl, or C₃₋₆ cycloalkyl;
   wherein, when R^{1c} is C₁₋₆ alkyl or C₃₋₆ cycloalkyl, then R^{1c} may be unsubstituted or substituted with one or more substituents, each of which independently is halogen, —OH, —N(R^{b})₂, C₁₋₃ alkyl, C₁₋₃ alkoxy, or C₁₋₆heteroalkyl;
R² is C₃₋₂₀ cycloalkyl, C₃₋₂₀ cycloalkenyl, C₆₋₁₈ aryl, heteroaryl having 5 to 20 ring members, or heterocyclyl having 3 to 20 ring members;
   wherein R² may be unsubstituted or substituted with one or more substituents, each of which independently is halogen, —OH, oxo, —N(R^{b})₂, C₁₋₆ alkyl, C₁₋₆ haloalkyl, C₁₋₆ alkoxy, C₁₋₆ alkylene-OH, C₁₋₆alkylene-O—C₁₋₆alkyl, or C₁₋₆heteroalkyl;
R³ is —H, C₁₋₆ alkyl, C₁₋₆ haloalkyl, C₃₋₆ cycloalkyl, amino, C₁₋₄alkyl-amino, di-C₁₋₄alkyl-amino, C₆₋₁₀ aryl, heteroaryl having 5 to 10 ring members, or heterocyclyl having 5 to 10 ring members;
   wherein, when R³ is C₁₋₆ alkyl, C₁₋₆ haloalkyl, C₃₋₆ cycloalkyl, C₁₋₄alkyl-amino, di-C₁₋₄alkyl-amino, C₆₋₁₀ aryl, heteroaryl having 5 to 10 ring members, or heterocyclyl having 5 to 10 ring members, then R³ may be unsubstituted or substituted with one or more substituents, each of which independently is halogen, —OH, oxo, —N(R^{b})₂, C₁₋₃ alkyl, C₁₋₃ haloalkyl, C₁₋₃ alkoxy, C₁₋₃ alkylene-C₁₋₃ alkoxy, C₁₋₆ heteroalkyl, or two R³ substituents together form a C₃₋₆ carbocyclyl or a heterocyclyl having 3 to 6 ring members;
R⁴ is —H, C₁₋₃ alkyl, C₁₋₃ haloalkyl, —CN, halogen, or —N(R^{a})₂ where each instance of R^{a}, where present, independently is —H, C₁₋₆ alkyl, or —C(O)C₁₋₆ alkyl; or, alternatively, two R^{a} substituents together form a heterocyclyl having 3 to 6 ring members;
   wherein R^{a} may be unsubstituted or substituted with one or more substituents, each of which independently is —F, —Cl, —OH, C₁₋₃ alkoxy; R⁵ is —H, C₁₋₆ alkyl, C₃₋₆ cycloalkyl, C₁₋₆ haloalkyl, or C₁₋₆heteroalkyl;
   wherein, when R⁵ is C₁₋₆ alkyl, C₃₋₆ cycloalkyl, C₁₋₆ haloalkyl, or C₁₋₆heteroalkyl, then R may be unsubstituted or substituted one or more substituents, each of which independently is halogen, —OH, C₁₋₃ alkyl, or C₁₋₃ alkoxy;
where each instance of R^{b}, where present, independently is —H or C₁₋₆ alkyl; or, alternatively, two R^{b} substituents together form a heterocyclyl having 3 to 6 ring members; and
wherein, when R^{b} is C₁₋₆ alkyl or, alternatively, two R^{b} substituents together form a heterocyclyl having 3 to 6 ring members, then R^{b} may be unsubstituted or substituted with one or more substituents, each of which independently is —F, —Cl, —OH, C₁₋₃ alkoxy. In several embodiments, R^{1a} and R^{1b} are not both methyl. In several embodiments, R^{1a} and R^{1b} are not both —CF₃. In several embodiments, R^{1a} is not methyl where R^{1b} is CF₃. In several embodiments, R^{1a} and R^{1b} do not together provide oxetanyl. In several embodiments, R^{1a} and R^{1b} do not together provide piperadinyl. In several embodiments, R³ is not unsubstituted or substituted morpholinyl. In several embodiments, R² is not unsubstituted or substituted phenyl. In several embodiments, R² is not unsubstituted or substituted heteroaryl having 6 ring members. In several embodiments, the structure of Formula (A) is further represented by the structure of Formula (I) (for example, where Z is C—R⁵, Y is C—R³, m is 1, and

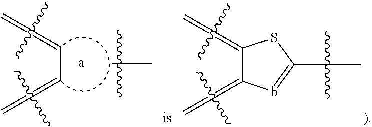

In several embodiments, R^{1a} and R^{1b} are not both H. In several embodiments, R^{1a} is —H, C₁₋₃ alkyl, or C₁₋₃ haloalkyl. In several embodiments, R^{1a} is C₁₋₆alkyl or C₁₋₆haloalkyl. In several embodiments, R^{1a} is —H or C₁₋₆alkyl. In several embodiments, R^{1a} is —H or C₁₋₆ haloalkyl. In several embodiments, R^{1a} is C₁₋₆ alkyl. In several embodiments, R^{1a} is C₁₋₆ haloalkyl. In several embodiments, R^{1a} is —H. In several embodiments, R^{1b} is C₁₋₆ alkyl, C₁₋₆ haloalkyl, C₃₋₁₀ cycloalkyl, or heterocyclyl having 3 to 10 ring members. In several embodiments, R^{1b} is C₁₋₆ alkyl or C₁₋₆ haloalkyl. In several embodiments, R^{1b} is C₃₋₁₀ cycloalkyl or heterocyclyl having 3 to 10 ring members. In several embodiments, R^{1b} is C₁₋₆haloalkyl. In several embodiments, R^{1b} is C₃₋₁₀ cycloalkyl. In several embodiments, R^{1b} is heterocyclyl having 3 to 10 ring members. In several embodiments, when R^{1b} is C₁₋₆ alkyl, C₁₋₆ haloalkyl, C₁₋₆ heteroalkyl, —(C₁₋₆ alkylene)-O—(C₁₋₆ alkyl), C₃₋₁₀ cycloalkyl, or heterocyclyl having 3 to 10 ring members, then R^{1b} may be unsubstituted or substituted with one or more substituents, each of which independently is halogen, -OH, —N(R^{b})₂, C₁₋₃ alkyl, or C₁₋₃ alkoxy; wherein each of the C₁₋₃ alkyl and C₁₋₃ alkoxy may be unsubstituted or substituted with 1 to 6 halogen groups (e.g., —F). In several embodiments, R^{1a} and R^{1b}together form a C₃₋₁₀ cycloalkyl or heterocyclyl having 3 to 10 ring members. In several embodiments, R^{1a} and R^{1b} together form a C₃₋₆ cycloalkyl. In several embodiments, R^{1a} and R^{1b} together form a C₄₋₆ cycloalkyl. In several embodiments, R^{1a} and R^{1b} together form a heterocyclyl having 4 to 6 ring members. In several embodiments, the R^{1a} and R^{1b} cycle may be unsubstituted or substituted with one or more substituents, each of which independently is halogen, —OH, oxo, —N(R^{b})₂, C₁₋₃ alkyl, or C₁₋₃alkoxy. In several embodiments, R^{1c} is —H. In several embodiments, R² is C₃₋₁₀ cycloalkyl, C₆₋₁₀ aryl, heteroaryl having 5 to 10 ring members, or heterocyclyl having 3 to 10 ring members. In several embodiments, R² is heteroaryl having 5 to 10 ring members or heterocyclyl having 3 to 10 ring members. In several embodiments, R² is heteroaryl having 5 to 10 ring members. In several embodiments, R³ is C₁₋₆ alkyl, C₁₋₆ haloalkyl, C₃₋₆ cycloalkyl, amino, C₁₋₄alkyl-amino, di-C₁₋₄alkyl-amino, C₆₋₁₀ aryl, heteroaryl having 5 to 10 ring members, or heterocyclyl having 5 to 10 ring members. In several embodiments, R³ is C₁₋₆ alkyl, C₁₋₆ haloalkyl, C₃₋₆ cycloalkyl, amino, C₁₋₂ alkyl-amino (e.g., -NH(C₁₋₂ alkyl)), di-C₁₋₂alkyl-amino (e.g., -N(C₁₋₂ alkyl)₂), C₆₋₁₀ aryl, heteroaryl having 5 to 10 ring members, or heterocyclyl having 5 to 10 ring members. In several embodiments, R³ is C₃₋₆ cycloalkyl, di-C₁₋₄alkyl-amino, C₆₋₁₀ aryl, heteroaryl having 5 to 10 ring members, or heterocyclyl having 5 to 10 ring members. In several embodiments, R³ is C₃₋₆ cycloalkyl, di-C₁₋₄alkyl-amino, heteroaryl having 5 to 10 ring members, or heterocyclyl having 5 to 10 ring members. In several embodiments, R³ is C₃₋₆ cycloalkyl, di-C₁₋₄alkyl-amino, C₆₋₁₀ aryl, or heteroaryl having 5 to 10 ring members. In several embodiments, R³ is C₃₋₆ cycloalkyl. In several embodiments, R³ is —N(Me)₂. In several embodiments, R³ is heteroaryl having 5 to 10 ring members. In several embodiments, R⁴ is —H, C₁₋₃ alkyl, halogen, or —N(R^{a})₂. In several embodiments, R⁴ is —H or C₁₋₃ alkyl. In several embodiments, R⁴ is —H or —N(R^{a})₂. In several embodiments, R⁴ is N(R^{a})₂, R⁴ may be acylated (e.g., —NHAc). In several embodiments, R⁴ may be CN. In several embodiments, R³ is —H, C₁₋₆ alkyl, or C₁₋₆ heteroalkyl. In several embodiments, R⁵ is —H, C₁₋₃ alkyl, or C₁₋₃ heteroalkyl. In several embodiments, R⁵ is —H. In several embodiments, R⁵ is C₁₋₆ alkyl. In several embodiments, R³ is C₁₋₆ heteroalkyl. In several embodiments, where R³ is a group other than —H, R³ is —H. In several embodiments, where R³ is a group other than —H, R⁵ is —H. In several embodiments, each of R³ and R³ is —H. In several embodiments, a haloalkyl as provided in a variable of Embodiment 1 (or any of the other embodiments provided herein) may be perhalogenated, where each —H of the haloalkyl chain has been exchanged for a halogen. In several embodiments, a haloalkyl as provided in a variable of Embodiment 1 (or any of the other embodiments provided herein) is not perhalogenated and one or more instances C—H occur (e.g., 1, 2, 3, 4, 5, 6, 7, 8, or more). In several embodiments, C—H instances occurring within the haloalkyl may occur on the same carbon (e.g., providing CH₂ or CH₃). In several embodiments, the haloalkyl includes at least one and optionally more instances of a halogen (e.g., 1, 2, 3, 4, 5, 6, 7, 8, 9, 10, 11, more instances, or perhalo). In several embodiments, each halogen of the haloalkyl independently is —F, —Cl, —Br, or —I. In several embodiments, each halogen of the haloalkyl is —F or —Cl. In several embodiments, each halogen of the haloalkyl is —F. In several embodiments, a heteroalkyl as provided in a variable of Embodiment 1 (or any of the other embodiments provided herein) includes a heteroatom (or one or more heteroatoms) within the alkyl backbone. In several embodiments, each heteroatom of the heteroalkyl independently is nitrogen, oxygen, or sulfur (e.g., S, SO, or SO₂). In several embodiments, each heteroatom of the heteroalkyl independently is nitrogen or oxygen. In several embodiments, each heteroatom of the heteroalkyl is nitrogen. In several embodiments, each heteroatom of the heteroalkyl is oxygen. In several embodiments, the heteroalkyl may have from 1 to 4 heteroatoms (e.g., 1, 2, 3, or 4), from 1 to 3 heteroatoms (e.g., 1, 2, or 3), 1 or 2 heteroatoms, or 1 heteroatom. In several embodiments, the compound of Formula (A) or a pharmaceutically acceptable salt of said compound is further represented by the compound of Formula (I) or a pharmaceutically acceptable salt thereof. For example, the structure of Formula (A) is further represented by the structure of Formula (I), where Z is C—R⁵, Y is C—R³, m is 1, and

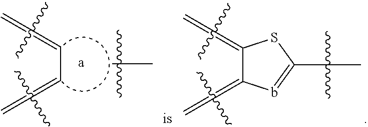

In several embodiments, R^{1a} and R^{1b} may both be —H. In several embodiments, R^{1a} and R^{1b} are not both —H.

A compound Formula (A) or pharmaceutically acceptable salt thereof may be further represented a structure of any one of the following formulae:

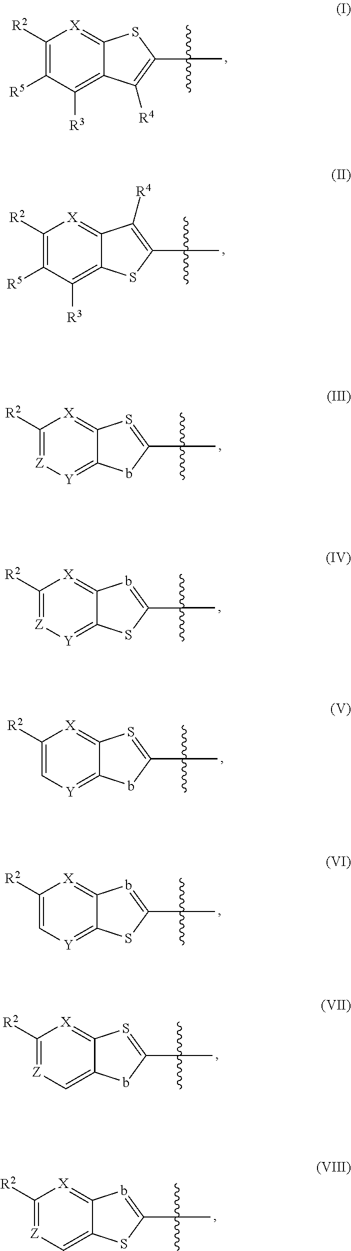

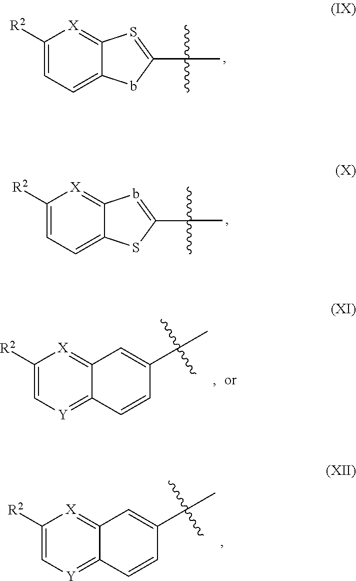

where the variables each formula are as provided in Formula (A) or as disclosed anywhere else herein.

A compound of Formula (A) or pharmaceutically acceptable salt thereof may be further represented by a structure of any one of the following formulae:

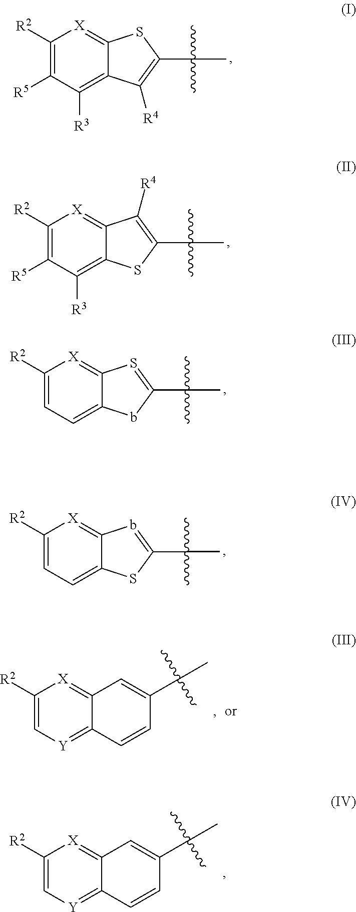

where the variables each formula are as provided in Formula (A) or as disclosed anywhere else herein (e.g., for any other formula provided herein).

Provided herein as Embodiment 2 is a compound of Formula (A):

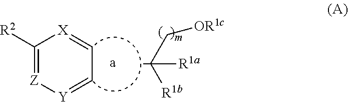

or a pharmaceutically acceptable salt of said compound;
wherein

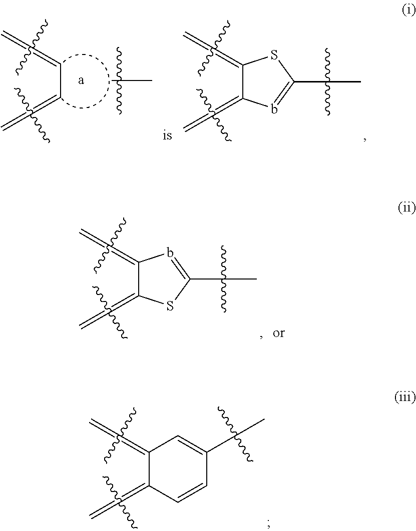

m is 0 or 1;
X is N or CH;
Y is N or C—R³;
Z is N or C-R⁵;
b is N or C-R⁴;
R^{1a} is —H, deuterium, C₁₋₆ alkyl, or C₁₋₆ haloalkyl;
   wherein, when R^{1a} is C₁₋₆ alkyl or C₁₋₆ haloalkyl, then R^{1a} may be unsubstituted or substituted with one or more substituents, each of which independently is halogen, —OH, —N(R^{b})₂, C₁₋₃ alkyl, or C₁₋₃ alkoxy;
R^{1b} is —H, C₁₋₆ alkyl, C₁₋₆ haloalkyl, —(C₁₋₆ alkylene)-O—(C₁₋₆ alkyl), C₃₋₁₀ cycloalkyl, or heterocyclyl having 3 to 10 ring members and 1 to 3 heteroatom ring members, each of which independently is nitrogen, oxygen, or —S(O)ₓ—, where x is 0, 1, or 2;
   wherein, when R^{1b} is C₁₋₆ alkyl, C₁₋₆ haloalkyl, —(C₁₋₆ alkylene)-O-(C₁₋₆ alkyl), C₃₋₁₀ cycloalkyl, or heterocyclyl having 3 to 10 ring members, then R^{1b} may be unsubstituted or substituted with one or more substituents, each of which independently is halogen, —OH, —N(R^{b})₂, C₁₋₃ alkyl, or C₁₋₃ alkoxy; wherein each of the C₁₋₃ alkyl and C₁₋₃ alkoxy may be unsubstituted or substituted with 1 to 6 F groups;
or, alternatively, R^{1a} and R^{1b} together form a C₃₋₁₀ cycloalkyl, C₃₋₁₀ cycloalkenyl, or heterocyclyl having 3 to 10 ring members and 1 to 3 heteroatom ring members, each of which independently is nitrogen, oxygen, or —S(O)ₓ—, where x is 0, 1, or 2;
   wherein the R^{1a} and R^{1b} cycle may be unsubstituted or substituted with one or more substituents, each of which independently is halogen, —OH, oxo, —N(R^{b})₂, C₁₋₃ alkyl, C₁₋₃alkoxy, -CN, C₃₋₁₀ cycloalkyl, or heterocyclyl having 3 to 10 ring members and 1 to 3 heteroatom ring members, each of which independently is nitrogen, oxygen, or —S(O)_{W}—, where w is 0, 1, or 2; wherein each of the C₁₋₃ alkyl and C₁₋₃ alkoxy may be unsubstituted or substituted with 1 to 9 halogen groups or C₁₋₃ alkoxy;
R^{1c} is —H, C₁₋₆ alkyl, or C₃₋₆ cycloalkyl;
   wherein, when R^{1c} is C₁₋₆ alkyl or C₃₋₆ cycloalkyl, then R^{1c} may be unsubstituted or substituted with one or more substituents, each of which independently is halogen, —OH, —N(R^{b})₂, C₁₋₃ alkyl, or C₁₋₃ alkoxy;
R² is C₃₋₂₀ cycloalkyl, C₃₋₂₀ cycloalkenyl, C₆₋₁₈ aryl, heteroaryl having 5 to 20 ring members and 1 to 5 heteroatoms each of which independently is nitrogen, oxygen, or —S(O)_{y}—, where y is 0 to 2, or heterocyclyl having 3 to 20 ring members and 1 to 5 heteroatoms each of which independently is nitrogen, oxygen, or —S(O)_{y}—, where y is 0 to 2;
   wherein R² may be unsubstituted or substituted with one or more substituents, each of which independently is halogen, —OH, oxo, —N(R^{b})₂, C₁₋₆ alkyl, C₁₋₆ haloalkyl, C₁₋₆ alkoxy, C₁₋₆alkylene-OH, or C₁₋₆alkylene-O—C₁₋₆alkyl;
R³ is —H, C₁₋₆ alkyl, C₁₋₆ haloalkyl, C₃₋₆ cycloalkyl, amino, C₁₋₄alkyl-amino, di-C₁₋₄alkyl-amino, C₆₋₁₀ aryl, heteroaryl having 5 to 10 ring members and 1 to 5 heteroatoms each of which independently is nitrogen, oxygen, or —S(O)_{z}—, where z is 0 to 2, or heterocyclyl having 5 to 10 ring members and 1 to 5 heteroatoms each of which independently is nitrogen, oxygen, or —S(O)_{z}—, where z is 0 to 2;
   wherein, when R³ is C₁₋₆ alkyl, C₁₋₆ haloalkyl, C₃₋₆ cycloalkyl, C₁₋₄alkyl-amino, di-C₁₋₄ alkyl-amino, C₆₋₁₀ aryl, heteroaryl having 5 to 10 ring members, or heterocyclyl having 5 to 10 ring members, then R³ may be unsubstituted or substituted with one or more substituents, each of which independently is halogen, —OH, oxo, —N(R^{b})₂, C₁₋₃ alkyl, C₁₋₃ haloalkyl, C₁₋₃ alkoxy, or C₁₋₃ alkylene-C₁₋₃ alkoxy;
R⁴ is —H, C₁₋₃ alkyl, halogen, or —N(R^{a})₂ where each instance of R^{a}, where present, independently is —H or C₁₋₆ alkyl; or, alternatively, two R^{a} substituents together form a heterocyclyl having 3 to 6 ring members and 1 to 2 heteroatom ring members, each of which independently is nitrogen or oxygen;
   wherein R^{a} may be unsubstituted or substituted with one or more substituents, each of which independently is —F, —Cl, —OH, C₁₋₃ alkoxy; R⁵ is —H, C₁₋₆ alkyl, C₃₋₆ cycloalkyl, or C₁₋₆ haloalkyl;
   wherein, when R is C₁₋₆ alkyl, C₃₋₆ cycloalkyl, or C₁₋₆ haloalkyl, then R² may be unsubstituted or substituted one or more substituents, each of which independently is halogen, —OH, C₁₋₃ alkyl, or C₁₋₃ alkoxy;
where each instance of R, where present, independently is —H or C₁₋₆ alkyl; or, alternatively, two R^{b} substituents together form a heterocyclyl having 3 to 6 ring members and 1 to 2 heteroatoms each of which independently is nitrogen or oxygen; and
wherein, when R^{b} is C₁₋₆ alkyl or, alternatively, two R^{b} substituents together form a heterocyclyl having 3 to 6 ring members, then R^{b} may be unsubstituted or substituted with one or more substituents, each of which independently is —F, —Cl, —OH, C₁₋₃ alkoxy.

Provided herein as Embodiment 3 is the compound or salt of Embodiment 1 or 2, wherein, when the compound of Formula (A) is represented by

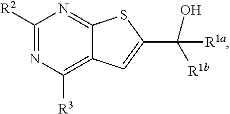

where b is C—H or N, R² is unsubstituted or substituted pyridinyl, unsubstituted or substituted pyrimidinyl, unsubstituted or substituted pyrrolopyridinyl, unsubstituted or substituted indazolyl, unsubstituted or substituted imidazopyridinyl, or unsubstituted or substituted quinolinyl, and 1) R^{1a} and R^{1b} are both methyl or 2) R^{1a} and R^{1b} together provide oxetanyl, then R³ is not morpholinyl.

Provided herein as Embodiment 4 is the compound or salt of Embodiment 1 to 3, wherein, when the compound of Formula (A) is represented by

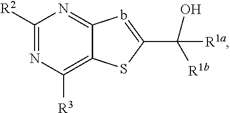

where b is C—H or N, R² is unsubstituted or substituted phenyl, unsubstituted or substituted piperadinyl, unsubstituted or substituted thiazolyl, unsubstituted or substituted pyridinyl, unsubstituted or substituted pyrimidinyl, unsubstituted or substituted 2, 3, 4, 5-tetrahydropyridinyl, unsubstituted or substituted indolyl, unsubstituted or substituted imidazopyridinyl, unsubstituted or substituted indazolyl, unsubstituted or substituted pyrrolopyridinyl, unsubstituted or substituted pyrrolopyridazinyl, unsubstituted or substituted quinolinyl, unsubstituted or substituted isoquinolinyl, or unsubstituted or substituted quinolinyl, and each of R^{1a} and R^{1b} independently is methyl, then R³ is not unsubstituted or substituted morpholinyl.

Provided herein as Embodiment 5 is the compound or salt of any one of Embodiments 1 to 4, wherein, when the compound of Formula (A) is represented by

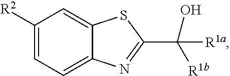

and R^{1a} and R^{1b}are both methyl, then R² is not unsubstituted or substituted phenyl, unsubstituted naphthalenyl, unsubstituted pyridinyl, unsubstituted benzothienyl, or unsubstituted phenanthrenyl.

Provided herein as Embodiment 6 is the compound or salt of any one of Embodiments 1 to 5, wherein, when the compound of Formula (A) is represented by

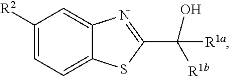

and R^{1a} and R^{1b}are both methyl, R^{1a} and R^{1b} are both —CF₃, R^{1a} is methyl and R^{1b} is CF₃, or R^{1a} and R^{1b} together provide unsubstituted or substituted piperdinyl, then R² is not unsubstituted or substituted phenyl, unsubstituted or substituted pyridinyl, unsubstituted or substituted 2, 3, 4, 5-tetrahydropyridinyl, unsubstituted or substituted pyrimidinyl, unsubstituted or substituted naphthalenyl, or unsubstituted or substituted benzothienyl.

Provided herein as Embodiment 7 is the compound or salt of any one of Embodiments 1 to 6, wherein, when the compound of Formula (A) is represented by

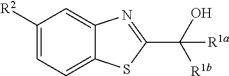

and R^{1a} and R^{1b}are both methyl or R^{1a} and R^{1b} together provide a unsubstituted or substituted piperadinyl, then R² is not unsubstituted or substituted piperadinyl, unsubstituted or substituted pyrimidinyl, or unsubstituted or substituted 2, 3, 4, 5-tetrahydropyridinyl.

Provided herein as Embodiment 8 is the compound or salt of any one of Embodiments 1 to 7, wherein, when the compound of Formula (A) is represented by

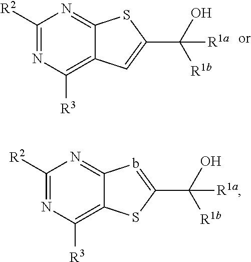

then R³ is not morpholinyl.

Provided herein as Embodiment 9 is the compound or salt of any one of Embodiments 1 to 8, wherein, when the compound of Formula (A) is represented by

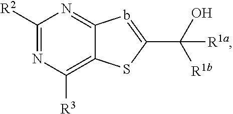

where b is C—H or N, R² is unsubstituted or substituted pyrimidinyl, unsubstituted or substituted 2, 3, 4, 5-tetrahydropyridinyl, or unsubstituted or substituted indazolyl, and 1) each of R^{1a} and R^{1b} independently is unsubstituted or substituted methyl, 2) each of R^{1a} and R^{1b} independently is unsubstituted or substituted ethyl, 3) R^{1a} and R^{1b} together provide unsubstituted or substituted piperdinyl, 4) R^{1a} and R^{1b} together provide oxetanyl, or 5) R^{1a} and R^{1b} together provide unsubstituted or substituted thiopyranyl, then R³ is not unsubstituted or substituted morpholinyl.

Provided herein as Embodiment 10 is the compound or salt of any one of Embodiments 1 to 9, wherein R³ is not unsubstituted or substituted morpholinyl.

Provided herein as Embodiment 11 is the compound or salt of any one of Embodiments 1 to 10, wherein R^{1a} is not —H.

Provided herein as Embodiment 12 is the compound or salt of any one of Embodiments 1 to 11, wherein R^{1b} is not —H.

Provided herein as Embodiment 13 is the compound or salt of any one of Embodiments 1 to 12, wherein

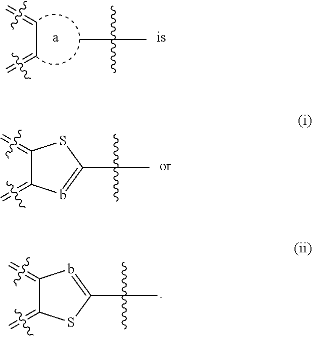

Provided herein as Embodiment 14 is the compound or salt of any one of Embodiments 1 to 12, wherein

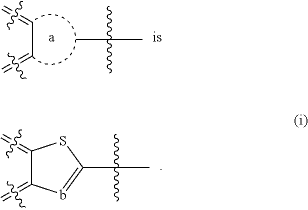

Provided herein as Embodiment 15 is the compound or salt of any one of Embodiments 1 to 12, wherein

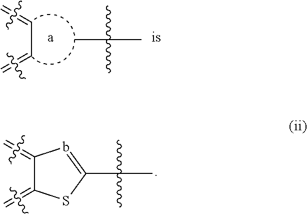

Provided herein as Embodiment 16 is the compound or salt of any one of Embodiments 1 to 12, wherein

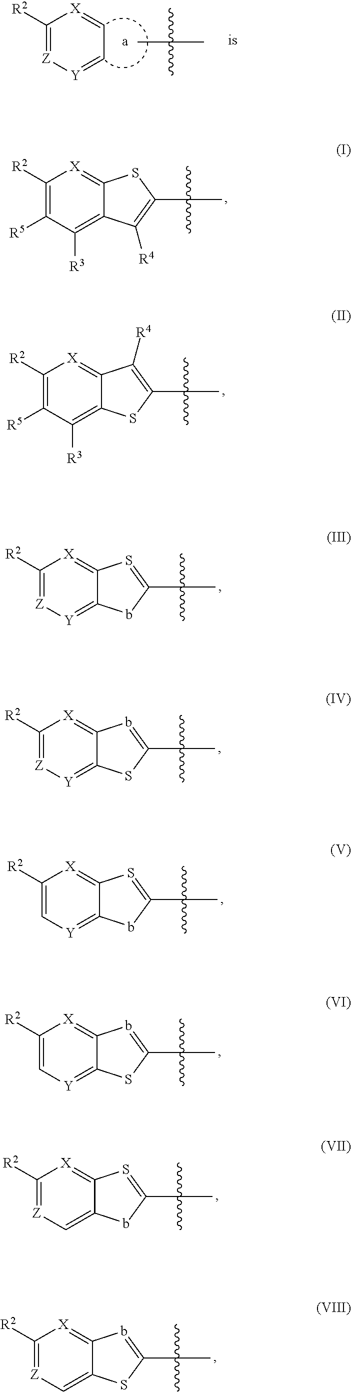

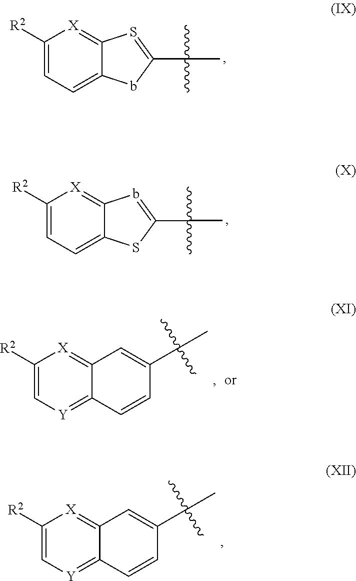

Provided herein as Embodiment 17 is the compound or salt of any one of Embodiments 1 to 16, wherein

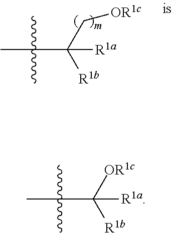

For example, in several embodiments, m is 0.

Provided herein as Embodiment 18 is the compound or salt of any one of Embodiments 1 to 16, wherein

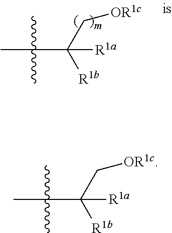

For example, in several embodiments, m is 1.

Provided herein as Embodiment 19 is the compound or salt of any one of Embodiments 1 to 18, wherein X is N.

Provided herein as Embodiment 20 is the compound or salt of any one of Embodiments 1 to 18, wherein X is CH.

Provided herein as Embodiment 21 is the compound or salt of any one of Embodiments 1 to 20, wherein Y is N.

Provided herein as Embodiment 22 is the compound or salt of any one of Embodiments 1 to 20, wherein Y is C—R³.

Provided herein as Embodiment 23 is the compound or salt of any one of Embodiments 1 to 22, wherein Z is N.

Provided herein as Embodiment 24 is the compound or salt of any one of Embodiments 1 to 22, wherein Z is C-R⁵.

Provided herein as Embodiment 25 is the compound or salt of any one of Embodiments 1 to 24, wherein b is N.

Provided herein as Embodiment 26 is the compound or salt of any one of Embodiments 1 to 24, wherein b is C—R⁴.

Provided herein as Embodiment 27 is a compound of Formula (I)

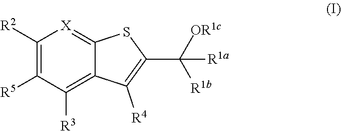

or a pharmaceutically acceptable salt of said compound;

where
X is N or CH;
R^{1a} is —H, C₁₋₆ alkyl, or C₁₋₆ haloalkyl;
   wherein, when R^{1a} is C₁₋₆ alkyl or C₁₋₆ haloalkyl, then R^{1a} may be unsubstituted or substituted with one or more substituents, each of which independently is halogen, —OH, —N(R^{b})₂, C₁₋₃ alkyl, or C₁₋₃ alkoxy;
R^{1b} is —H, C₁₋₆ alkyl, C₁₋₆ haloalkyl, C₁₋₆ heteroalkyl, —(C₁₋₆ alkylene)-O-(C₁₋₆ alkyl), C₃₋₁₀ cycloalkyl, or heterocyclyl having 3 to 10 ring members;
   wherein, when R^{1b} is C₁₋₆ alkyl, C₁₋₆ haloalkyl, C₁₋₆ heteroalkyl, C₃₋₁₀ cycloalkyl, or heterocyclyl having 3 to 10 ring members, then R^{1b} may be unsubstituted or substituted with one or more substituents, each of which independently is halogen, —OH, —N(R^{b})₂, C₁₋₃ alkyl, or C₁₋₃ alkoxy; wherein each of the C₁₋₃ alkyl and C₁₋₃ alkoxy may be unsubstituted or substituted with 1 to 6—F groups;
or, alternatively, R^{1a} and R^{1b} together form a C₃₋₁₀ cycloalkyl, C₃₋₁₀ cycloalkenyl, or heterocyclyl having 3 to 10 ring members;
   wherein the R^{1a} and R^{1b} cycle may be unsubstituted or substituted with one or more substituents, each of which independently is halogen, —OH, oxo, —N(R^{b})₂, C₁₋₃ alkyl, C₁₋₃alkoxy, or C₁₋₆heteroalkyl; wherein each of the C₁₋₃ alkyl and C₁₋₃ alkoxy may be unsubstituted or substituted with 1 to 9 halogen groups or C₁₋₃ alkoxy; R^{1c} is —H, C₁₋₆ alkyl, or C₃₋₆ cycloalkyl;
   wherein, when R^{1c} is C₁₋₆ alkyl or C₃₋₆ cycloalkyl, R^{1c} may be unsubstituted or substituted with one or more substituents, each of which independently is halogen, —OH, —N(R^{b})₂, C₁₋₃ alkyl, C₁₋₃ alkoxy, or C₁₋₆heteroalkyl;
R² is C₃₋₂₀ cycloalkyl, C₃₋₂₀ cycloalkenyl, C₆₋₁₈ aryl, heteroaryl having 5 to 20 ring members, or heterocyclyl having 3 to 20 ring members;
   wherein R² may be unsubstituted or substituted with one or more substituents, each of which independently is halogen, —OH, oxo, —N(R^{b})₂, C₁₋₆ alkyl, C₁₋₆ haloalkyl, C₁₋₆ alkoxy, C₁₋₆ alkylene-OH, C₁₋₆alkylene-O—C₁₋₆alkyl, or C₁₋₆heteroalkyl;
R³ is —H, C₁₋₆ alkyl, C₁₋₆ haloalkyl, C₃₋₆ cycloalkyl, amino, C₁₋₄alkyl-amino, di-C₁₋₄alkyl-amino, C₆₋₁₀ aryl, heteroaryl having 5 to 10 ring members, or heterocyclyl having 5 to 10 ring members;
   wherein, when R³ is C₁₋₆ alkyl, C₁₋₆ haloalkyl, C₃₋₆ cycloalkyl, C₁₋₄alkyl-amino, di-C₁₋₄ alkyl-amino, C₆₋₁₀ aryl, heteroaryl having 5 to 10 ring members, or heterocyclyl having 5 to 10 ring members, then R³ may be unsubstituted or substituted with one or more substituents, each of which independently is halogen, —OH, oxo, —N(R^{b})₂, C₁₋₃ alkyl, C₁₋₃ haloalkyl, C₁₋₃ alkoxy, C₁₋₃ alkylene-C₁₋₃ alkoxy, C₁₋₆ heteroalkyl, or two R³ substituents together form a C₃₋₆ carbocyclyl or a heterocyclyl having 3 to 6 ring members;
R⁴ is —H or —N(R^{a})₂ where each instance of R^{a}, where present, independently is —H or C₁₋₆ alkyl; or, alternatively, two R^{a} substituents together form a heterocyclyl having 3 to 6 ring members;
   wherein R^{a} may be unsubstituted or substituted with one or more substituents, each of which independently is —F, —Cl, —OH, C₁₋₃ alkoxy;
R⁵ is —H, C₁₋₆ alkyl, C₃₋₆ cycloalkyl, C₁₋₆ haloalkyl, or C₁₋₆heteroalkyl;
   wherein, when R³ is C₁₋₆ alkyl, C₃₋₆ cycloalkyl, C₁₋₆ haloalkyl, or C₁₋₆heteroalkyl, then R may be unsubstituted or substituted one or more substituents, each of which independently is halogen, —OH, C₁₋₃ alkyl, or C₁₋₃ alkoxy;
where each instance of R^{b}, where present, independently is —H or C₁₋₆ alkyl; or, alternatively, two R^{b} substituents together form a heterocyclyl having 3 to 6 ring members; and wherein, when R^{b} is C₁₋₆ alkyl or, alternatively, two R^{b} substituents together form a heterocyclyl having 3 to 6 ring members, then R^{b} may be unsubstituted or substituted with one or more substituents, each of which independently is —F, —Cl, —OH, C₁₋₃ alkoxy. In several embodiments, R^{1a} is —H, C₁₋₃ alkyl, or C₁₋₃ haloalkyl. In several embodiments, R^{1a} is C₁₋₆alkyl or C₁₋₆haloalkyl. In several embodiments, R^{1a} is —H or C₁₋₆alkyl. In several embodiments, R^{1a} is —H or C₁₋₆ haloalkyl. In several embodiments, R^{1a} is C₁₋₆ alkyl. In several embodiments, R^{1a} is C₁₋₆haloalkyl. In several embodiments, R^{1a} is —H. In several embodiments, R^{1b} is C₁₋₆ alkyl, C₁₋₆ haloalkyl, C₁₋₆ heteroalkyl, C₃₋₁₀ cycloalkyl, or heterocyclyl having 3 to 10 ring members. In several embodiments, R^{1b} is C₁₋₆ alkyl, C₁₋₆ haloalkyl, or C₁₋₆ heteroalkyl. In several embodiments, R^{1b} is C₃₋₁₀ cycloalkyl or heterocyclyl having 3 to 10 ring members. In several embodiments, R^{1b} is C₁₋₆ haloalkyl. In several embodiments, R^{1b} is C₃₋₁₀ cycloalkyl. In several embodiments, R^{1b} is heterocyclyl having 3 to 10 ring members. In several embodiments, R^{1a} and R^{1b}together form a C₃₋₁₀ cycloalkyl or heterocyclyl having 3 to 10 ring members. In several embodiments, R^{1a} and R^{1b} together form a C₃₋₆ cycloalkyl. In several embodiments, R^{1a} and R^{1b} together form a C₄₋₆ cycloalkyl. In several embodiments, R^{1a} and R^{1b} together form a heterocyclyl having 4 to 6 ring members. In several embodiments, the R^{1a} and R^{1b} cycle may be unsubstituted or substituted with one or more substituents, each of which independently is halogen, —OH, oxo, —N(R^{b})₂, C₁₋₃ alkyl, or C₁₋₃alkoxy. In several embodiments, R^{1c} is —H. In several embodiments, R² is C₃₋₁₀ cycloalkyl, C₆₋₁₀ aryl, heteroaryl having 5 to 10 ring members, or heterocyclyl having 3 to 10 ring members. In several embodiments, R² is heteroaryl having 5 to 10 ring members or heterocyclyl having 3 to 10 ring members. In several embodiments, R² is heteroaryl having 5 to 10 ring members. In several embodiments, R³ is C₁₋₆ alkyl, C₁₋₆ haloalkyl, C₃₋₆ cycloalkyl, amino, C₁₋₄alkyl-amino, di-C₁₋₄alkyl-amino, C₆₋₁₀ aryl, heteroaryl having 5 to 10 ring members, or heterocyclyl having 5 to 10 ring members. In several embodiments, R³ is C₁₋₆ alkyl, C₁₋₆ haloalkyl, C₃₋₆ cycloalkyl, amino, C₁₋₂ alkyl-amino (e.g., —NH(C₁₋₂ alkyl)), di-C₁₋₂alkyl-amino (e.g., -N(C₁₋₂ alkyl)₂), C₆₋₁₀ aryl, heteroaryl having 5 to 10 ring members, or heterocyclyl having 5 to 10 ring members. In several embodiments, R³ is C₃₋₆ cycloalkyl, di-C₁₋₄alkyl-amino, C₆₋₁₀ aryl, heteroaryl having 5 to 10 ring members, or heterocyclyl having 5 to 10 ring members. In several embodiments, R³ is C₃₋₆ cycloalkyl, di-C₁₋₄alkyl-amino, heteroaryl having 5 to 10 ring members, or heterocyclyl having 5 to 10 ring members. In several embodiments, R³ is C₃₋₆ cycloalkyl, di-C₁₋₄alkyl-amino, C₆₋₁₀ aryl, or heteroaryl having 5 to 10 ring members. In several embodiments, R³ is C₃₋₆ cycloalkyl. In several embodiments, R³ is —N(Me)₂. In several embodiments, R³ is heteroaryl having 5 to 10 ring members. In several embodiments, R⁴ is —H or —N(R^{a})₂. In several embodiments, R⁴ is N(R^{a})₂₂, R⁴ may be acylated (e.g., -NHAc). In several embodiments, R⁴ may be CN. In several embodiments, R is —H, C₁₋₆ alkyl, or C₁₋₆ heteroalkyl. In several embodiments, R is —H, C₁₋₃ alkyl, or C₁₋₃ heteroalkyl. In several embodiments, R⁵ is —H. In several embodiments, R is C₁₋₆ alkyl. In several embodiments, R is C₁₋₆ heteroalkyl. In several embodiments, where R is a group other than —H, R³ is —H. In several embodiments, where R³ is a group other than —H, R is —H. In several embodiments, each of R³ and R is —H. In several embodiments, a haloalkyl as provided in a variable of Embodiment 27 (or any of the other embodiments provided herein) may be perhalogenated, where each —H of the haloalkyl chain has been exchanged for a halogen. In several embodiments, a haloalkyl as provided in a variable of Embodiment 27 (or any of the other embodiments provided herein) is not perhalogenated and one or more instances C—H occur (e.g., 1, 2, 3, 4, 5, 6, 7, 8, or more). In several embodiments, C—H instances occurring within the haloalkyl may occur on the same carbon (e.g., providing CH₂ or CH₃). In several embodiments, the haloalkyl includes at least one and optionally more instances of a halogen (e.g., 1, 2, 3, 4, 5, 6, 7, 8, 9, 10, 11, more instances, or perhalo). In several embodiments, each halogen of the haloalkyl independently is —F, —Cl, —Br, or —I. In several embodiments, each halogen of the haloalkyl is —F or —Cl. In several embodiments, each halogen of the haloalkyl is —F. In several embodiments, a heteroalkyl as provided in a variable of Embodiment 27 (or any of the other embodiments provided herein) includes a heteroatom (or one or more heteroatoms) within the alkyl backbone. In several embodiments, each heteroatom of the heteroalkyl independently is nitrogen, oxygen, or sulfur (e.g., S, SO, or SO₂). In several embodiments, each heteroatom of the heteroalkyl independently is nitrogen or oxygen. In several embodiments, each heteroatom of the heteroalkyl is nitrogen. In several embodiments, each heteroatom of the heteroalkyl is oxygen. In several embodiments, the heteroalkyl may have from 1 to 4 heteroatoms (e.g., 1, 2, 3, or 4), from 1 to 3 heteroatoms (e.g., 1, 2, or 3), 1 or 2 heteroatoms, or 1 heteroatom. In several embodiments, the compound of Formula (A) or a pharmaceutically acceptable salt of said compound is further represented by the compound of Formula (I) or a pharmaceutically acceptable salt thereof. For example, the structure of Formula (A) is further represented by the structure of Formula (I), where Z is C-R, Y is C—R³, m is 1, and

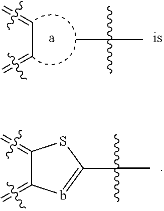

In several embodiments, R^{1a} and R^{1b} may both be —H. In several embodiments, R^{1a} and R^{1b} are not both —H.

Provided herein as Embodiment 28 is a compound of Formula (I)

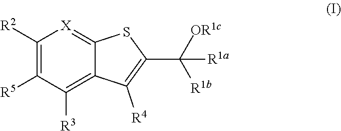

or a pharmaceutically acceptable salt of said compound;

where
X is N or CH;
R^{1a} is —H, C₁₋₆ alkyl, or C₁₋₆ haloalkyl;
   wherein, when R^{1a} is C₁₋₆ alkyl or C₁₋₆ haloalkyl, then R^{1a} may be unsubstituted or substituted with one or more substituents, each of which independently is halogen, —OH, —N(R^{b})₂, C₁₋₃ alkyl, or C₁₋₃ alkoxy;
R^{1b} is —H, C₁₋₆ alkyl, C₁₋₆ haloalkyl, —(C₁₋₆ alkylene)-O—(C₁₋₆ alkyl), C₃₋₁₀ cycloalkyl, or heterocyclyl having 3 to 10 ring members and 1 to 3 heteroatom ring members, each of which independently is nitrogen, oxygen, or —S(O)ₓ—, where x is 0, 1, or 2;
   wherein, when R^{1b} is C₁₋₆ alkyl, C₁₋₆ haloalkyl, —(C₁₋₆ alkylene)-O-(C₁₋₆ alkyl), C₃₋₁₀ cycloalkyl, or heterocyclyl having 3 to 10 ring members, then R^{1b} may be unsubstituted or substituted with one or more substituents, each of which independently is halogen, —OH, —N(R^{b})₂, C₁₋₃ alkyl, or C₁₋₃ alkoxy; wherein each of the C₁₋₃ alkyl and C₁₋₃ alkoxy may be unsubstituted or substituted with 1 to 6—F groups;
or, alternatively, R^{1a} and R^{1b} together form a C₃₋₁₀ cycloalkyl, C₃₋₁₀ cycloalkenyl, or heterocyclyl having 3 to 10 ring members and 1 to 3 heteroatom ring members, each of which independently is nitrogen, oxygen, or —S(O)ₓ—, where x is 0, 1, or 2;
   wherein the R^{1a} and R^{1b} cycle may be unsubstituted or substituted with one or more substituents, each of which independently is halogen, —OH, oxo, —N(R^{b})₂, C₁₋₃ alkyl, or C₁₋₃alkoxy; wherein each of the C₁₋₃ alkyl and C₁₋₃ alkoxy may be unsubstituted or substituted with 1 to 9 halogen groups or C₁₋₃ alkoxy; R^{1c} is —H, C₁₋₆ alkyl, or C₃₋₆ cycloalkyl;
   wherein, when R^{1c} is C₁₋₆ alkyl or C₃₋₆ cycloalkyl, R^{1c} may be unsubstituted or substituted with one or more substituents, each of which independently is halogen, —OH, —N(R^{b})₂, C₁₋₃ alkyl, or C₁₋₃ alkoxy;
R² is C₃₋₂₀ cycloalkyl, C₃₋₂₀ cycloalkenyl, C₆₋₁₈ aryl, heteroaryl having 5 to 20 ring members and 1 to 5 heteroatoms each of which independently is nitrogen, oxygen, or —S(O)_{y}—, where y is 0 to 2, or heterocyclyl having 3 to 20 ring members and 1 to 5 heteroatoms each of which independently is nitrogen, oxygen, or —S(O)_{y}—, where y is 0 to 2;
   wherein R² may be unsubstituted or substituted with one or more substituents, each of which independently is halogen, —OH, oxo, —N(R^{b})₂, C₁₋₆ alkyl, C₁₋₆ haloalkyl, C₁₋₆ alkoxy, C₁₋₆alkylene-OH, or —C₁₋₆alkylene-O—C₁₋₆alkyl;
R³ is —H, C₁₋₆ alkyl, C₁₋₆ haloalkyl, C₃₋₆ cycloalkyl, amino, C₁₋₄alkyl-amino, di-C₁₋₄alkyl-amino, C₆₋₁₀ aryl, heteroaryl having 5 to 10 ring members and 1 to 5 heteroatoms each of which independently is nitrogen, oxygen, or —S(O)_{z}—, where z is 0 to 2, or heterocyclyl having 5 to 10 ring members and 1 to 5 heteroatoms each of which independently is nitrogen, oxygen, or —S(O)_{z}—, where z is 0 to 2;
   wherein, when R³ is C₁₋₆ alkyl, C₁₋₆ haloalkyl, C₃₋₆ cycloalkyl, C₁₋₄alkyl-amino, di-C₁₋₄ alkyl-amino, C₆₋₁₀ aryl, heteroaryl having 5 to 10 ring members, or heterocyclyl having 5 to 10 ring members, then R³ may be unsubstituted or substituted with one or more substituents, each of which independently is halogen, —OH, oxo, —N(R^{b})₂, C₁₋₃ alkyl, C₁₋₃ haloalkyl, C₁₋₃ alkoxy, C₁₋₃ alkylene-C₁₋₃ alkoxy, C₁₋₆ heteroalkyl, or two R³ substituents together form a C₃₋₆ carbocyclyl or a heterocyclyl having 3 to 6 ring members and 1 to 2 heteroatom ring members, each of which independently is nitrogen or oxygen;
R⁴ is —H or —N(R^{a})₂ where each instance of R, where present, independently is —H or C₁₋₆ alkyl; or, alternatively, two R^{a} substituents together form a heterocyclyl having 3 to 6 ring members and 1 to 2 heteroatom ring members, each of which independently is nitrogen or oxygen;
   wherein R^{a} may be unsubstituted or substituted with one or more substituents, each of which independently is —F, —Cl, —OH, C₁₋₃ alkoxy; R⁵ is —H, C₁₋₆ alkyl, C₃₋₆ cycloalkyl, or C₁₋₆ haloalkyl;
   wherein, when R is C₁₋₆ alkyl, C₃₋₆ cycloalkyl, or C₁₋₆ haloalkyl, then R² may be unsubstituted or substituted one or more substituents, each of which independently is halogen, —OH, C₁₋₃ alkyl, or C₁₋₃ alkoxy;
where each instance of R, where present, independently is —H or C₁₋₆ alkyl; or, alternatively, two R^{b} substituents together form a heterocyclyl having 3 to 6 ring members and 1 to 3 heteroatoms each of which independently is nitrogen or oxygen; and
wherein, when R^{b} is C₁₋₆ alkyl or, alternatively, two R^{b} substituents together form a heterocyclyl having 3 to 6 ring members, then R^{b} may be unsubstituted or substituted with one or more substituents, each of which independently is —F, —Cl, —OH, C₁₋₃ alkoxy.

Provided herein as Embodiment 29 is the compound or salt of Embodiment 27 or 28, wherein the compound of Formula (I) is not 6-phenyl-4-(trifluoromethyl)thieno[2,3-b]pyridine-2-methanol, 6-cyclopropylbenzo[b]thiophene-2-methanol, 6-phenylbenzo[b]thiophene-2-methanol, or 6-[3-[(dimethylamino)methyl]bicyclo[2.2.1]hept-2-en-2-yl]benzo[b]thiophene-2-methanol. For example, in several embodiments, the compound of Formula (I) is not 6-phenyl-4-(trifluoromethyl)thieno[2,3-b]pyridine-2-methanol. In several embodiments, the compound of Formula (I) is not 6-cyclopropylbenzo[b]thiophene-2-methanol. In several embodiments, the compound of Formula (I) is not 6-phenylbenzo[b]thiophene-2-methanol. In several embodiments, the compound of Formula (I) is not 6-[3-[(dimethylamino)methyl]bicyclo[2.2.1]hept-2-en-2-yl]benzo[b]thiophene-2-methanol.

Provided herein as Embodiment 30 is the compound or salt of any one of Embodiments 27 to 29, wherein R² is not unsubstituted phenyl.

Provided herein as Embodiment 31 is the compound or salt of any one of Embodiments 27 to 30, wherein when R² is unsubstituted cyclopropyl, unsubstituted phenyl, or unsubstituted or substituted cycloalkenyl, then one of R¹, R^{1b}, and R^{1c} is not —H.

Provided herein as Embodiment 32 is the compound or salt of any one of Embodiments 27 to 31, wherein X is N.

Provided herein as Embodiment 33 is the compound or salt of any one of Embodiments 27 to 31, wherein X is CH.

Provided herein as Embodiment 34 is the compound or salt of any one of Embodiments 1 to 33, wherein R^{1a} is —H, deuterium, unsubstituted or substituted C₁₋₃alkyl, or unsubstituted or substituted C₁₋₃ haloalkyl. For example, in several embodiments, R^{1a} is —H, unsubstituted or substituted C₁₋₃alkyl, or unsubstituted or substituted C₁₋₃haloalkyl. In several embodiments, R^{1a} is —H or unsubstituted or substituted C₁₋₃haloalkyl. In several embodiments, R^{1a} is unsubstituted or substituted C₁₋₃alkyl or unsubstituted or substituted C₁₋₃haloalkyl. In several embodiments, R^{1a} is —H, deuterium, or unsubstituted or substituted C₁₋₃haloalkyl. In several embodiments, R^{1a} is deuterium, unsubstituted or substituted C₁₋₃alkyl, or unsubstituted or substituted C₁₋₃haloalkyl. In several embodiments, R^{1a} is —H, deuterium, or unsubstituted or substituted C₁₋₃alkyl. In several embodiments, R^{1a} is —H or deuterium. In several embodiments, R^{1a} is unsubstituted or substituted C₁₋₂ alkyl. In several embodiments, R^{1a} is methyl. In several embodiments, R^{1a} is unsubstituted or substituted C₂ alkyl. In several embodiments, R^{1a} is unsubstituted or substituted C₃ alkyl. In several embodiments, R^{1a} is unsubstituted or substituted C₁₋₂ haloalkyl. In several embodiments, R^{1a} is unsubstituted or substituted C₁ haloalkyl (e.g., —CF₃). In several embodiments, R^{1a} is unsubstituted or substituted C₂haloalkyl. In several embodiments, R^{1a} is unsubstituted or substituted C₃ haloalkyl.

Provided herein as Embodiment 35 is the compound or salt of any one of Embodiments 1 to 33, wherein R^{1a} is unsubstituted or substituted C₁₋₃alkyl. For example, in several embodiments, R^{1a} is unsubstituted C₁₋₂ alkyl. In several embodiments, R^{1a} is unsubstituted C₂₋₃ alkyl. In several embodiments, R^{1a} is unsubstituted C₁ alkyl. In several embodiments, R^{1a} is unsubstituted C₂ alkyl. In several embodiments, R^{1a} is unsubstituted C₃ alkyl. In several embodiments, R^{1a} is substituted C₁₋₂ alkyl. In several embodiments, R^{1a} is substituted C₂₋₃ alkyl. In several embodiments, R^{1a} is substituted C₁ alkyl. In several embodiments, R^{1a} is substituted C₂ alkyl. In several embodiments, R^{1a} is substituted C₃ alkyl.

Provided herein as Embodiment 36 is the compound or salt of any one of Embodiments 1 to 33, wherein R^{1a} is unsubstituted or substituted C₁₋₃haloalkyl. For example, in several embodiments, R^{1a} is unsubstituted C₁₋₂haloalkyl. In several embodiments, R^{1a} is unsubstituted C₂₋₃ haloalkyl. In several embodiments, R^{1a} is unsubstituted C₁ haloalkyl (e.g., —CF₃). In several embodiments, R^{1a} is unsubstituted C₂haloalkyl. In several embodiments, R^{1a} is unsubstituted C₃ haloalkyl. In several embodiments, R^{1a} is substituted C₁2 haloalkyl. In several embodiments, R^{1a} is substituted C₂₋₃ haloalkyl. In several embodiments, R^{1a} is substituted C₁ haloalkyl (e.g., —CF₃). In several embodiments, R^{1a} is substituted C₂ haloalkyl. In several embodiments, R^{1a} is substituted C₃ haloalkyl.

Provided herein as Embodiment 37 is the compound or salt of any one of Embodiments 1 to 36, wherein R^{1a} is substituted with 1, 2, 3, or 4 substituents. For example, in several embodiments, R^{1a} is substituted with 1 to 4 substituents. In several embodiments, R^{1a} is substituted with 1 to 3 substituents. In several embodiments, R^{1a} is substituted with 1 to 2 substituents. In several embodiments, R^{1a} is substituted with 2 to 4 substituents. In several embodiments, R^{1a} is substituted with 3 to 4 substituents. In several embodiments, R^{1a} is substituted with 2 to 3 substituents.

Provided herein as Embodiment 38 is the compound or salt of any one of Embodiments 27 to 37, wherein R^{1a} is substituted with 1, 2, or 3 substituents.

Provided herein as Embodiment 39 is the compound or salt of any one of Embodiments 1 to 38, wherein R^{1a} is substituted with 1 or 2 substituents.

Provided herein as Embodiment 40 is the compound or salt of any one of Embodiments 1 to 39, wherein R^{1a} is substituted with 1 substituent.

Provided herein as Embodiment 41 is the compound or salt of any one of Embodiments 1 to 40, wherein, when R^{1a} is substituted, each R^{1a} substituent independently is —F, —OH, or C₁₋₃alkoxy. For example, in several embodiments, each R^{1a} substituent independently is —F or -OH. In several embodiments, each R^{1a} substituent independently is —OH or C₁₋₃ alkoxy. In several embodiments, each R^{1a} substituent independently is —F or C₁₋₃ alkoxy. In several embodiments, R^{1a} is substituted with —F, —OH, C₁ alkoxy, C₂ alkoxy, and/or C₃ alkoxy. In several embodiments, R^{1a} each R^{1a} substituent independently is —C₁ alkoxy, C₂ alkoxy, or C₃ alkoxy. In several embodiments, R^{1a} is substituted with -C₁ alkoxy. In several embodiments, R^{1a} is substituted with C₂ alkoxy. In several embodiments, R^{1a} is substituted with C₃ alkoxy. In several embodiments, R^{1a} is substituted with —F. In several embodiments, R^{1a} is substituted with -OH.

Provided herein as Embodiment 42 is the compound or salt of any one of Embodiments 1 to 36, wherein R^{1a} is unsubstituted.

Provided herein as Embodiment 43 is the compound or salt of any one of Embodiments 1 to 33, wherein R^{1a} is —H, methyl, or —CF₃. For example, in several embodiments, R^{1a} is —H or methyl. In several embodiments, R^{1a} is —H or —CF₃. In several embodiments, R^{1a} is methyl or —CF₃. In several embodiments, R^{1a} is —H. In several embodiments, R^{1a} is methyl. In several embodiments, R^{1a} is —CF₃.

Provided herein as Embodiment 44 is the compound or salt of any one of Embodiments 1 to 33, wherein when R^{1a} is —H.

Provided herein as Embodiment 45 is the compound or salt of any one of Embodiments 1 to 33, wherein when R^{1a} is —CH₃.

Provided herein as Embodiment 46 is the compound or salt of any one of Embodiments 1 to 33, wherein when R^{1a} is —CF₃.

Provided herein as Embodiment 47 is the compound or salt of any one of Embodiments 1 to 46, wherein R^{1b} is —H, unsubstituted or substituted C₁₋₃ alkyl, unsubstituted or substituted C₁₋₃ haloalkyl, unsubstituted or substituted C₃₋₆ cycloalkyl, or unsubstituted or substituted heterocyclyl having 3- to 6-ring members. For example, in several embodiments, R^{1b} is unsubstituted or substituted C₁₋₃ alkyl, unsubstituted or substituted C₁₋₃ haloalkyl, unsubstituted or substituted C₃₋₆ cycloalkyl, or unsubstituted or substituted heterocyclyl having 3- to 6-ring members. In several embodiments, R^{1b} is unsubstituted or substituted C₁₋₃ haloalkyl, unsubstituted or substituted C₃₋₆ cycloalkyl, or unsubstituted or substituted heterocyclyl having 3- to 6-ring members. In several embodiments, R^{1b} is unsubstituted or substituted C₁₋₃ alkyl, unsubstituted or substituted C₃₋₆ cycloalkyl, or unsubstituted or substituted heterocyclyl having 3- to 6-ring members. In several embodiments, R^{1b} is unsubstituted or substituted C₁₋₃ alkyl, unsubstituted or substituted C₁₋₃ haloalkyl, or unsubstituted or substituted C₃₋₆ cycloalkyl. In several embodiments, R^{1b} is unsubstituted or substituted C₁₋₃ alkyl or unsubstituted or substituted C₃₋₆ cycloalkyl. In several embodiments, R^{1b} is unsubstituted or substituted C₁₋₃ alkyl or unsubstituted or substituted C₁₋₃ haloalkyl. In several embodiments, R^{1b} is unsubstituted or substituted C₃₋₆ cycloalkyl or heterocyclyl having 3- to 6-ring members. In several embodiments, R^{1b} is unsubstituted or substituted C₁₋₃ haloalkyl or unsubstituted or substituted heterocyclyl having 3- to 6-ring members. In several embodiments, R^{1b} is unsubstituted or substituted C₁₋₃ haloalkyl or unsubstituted or substituted C₃₋₆ cycloalkyl. In several embodiments, where R^{1b} is unsubstituted or substituted alkyl, R^{1b} is unsubstituted or substituted C₁₋₂ alkyl. In several embodiments, where R^{1b} is unsubstituted or substituted alkyl, R^{1b} is unsubstituted or substituted C₂₋₃ alkyl. In several embodiments, where R^{1b} is unsubstituted or substituted alkyl, R^{1b} is unsubstituted or substituted C₁ alkyl (e.g., methyl). In several embodiments, where R^{1b} is unsubstituted or substituted alkyl, R^{1b} is unsubstituted or substituted C₂ alkyl (e.g., ethyl). In several embodiments, where R^{1b} is unsubstituted or substituted alkyl, R^{1b} is unsubstituted or substituted C₃ alkyl (e.g., isopropyl or propyl). In several embodiments, where R^{1b} is unsubstituted or substituted haloalkyl, R^{1b} is unsubstituted or substituted C₁₋₂ haloalkyl. In several embodiments, where R^{1b} is unsubstituted or substituted haloalkyl, R^{1b} is unsubstituted or substituted C₂₋₃ haloalkyl. In several embodiments, where R^{1b} is unsubstituted or substituted haloalkyl, R^{1b} is unsubstituted or substituted C₁ haloalkyl (e.g., —CF₃, etc.). In several embodiments, where R^{1b} is unsubstituted or substituted haloalkyl, R^{1b} is unsubstituted or substituted C₂ haloalkyl. In several embodiments, where R^{1b} is unsubstituted or substituted haloalkyl, R^{1b} is unsubstituted or substituted C₃ haloalkyl (e.g., —CH₂CH₂CF₃, etc.). In several embodiments, where R^{1b} is unsubstituted or substituted cycloalkyl, R^{1b} is unsubstituted or substituted C₄₋₆ cycloalkyl. In several embodiments, where R^{1b} is unsubstituted or substituted cycloalkyl, R^{1b} is unsubstituted or substituted C₃-s cycloalkyl. In several embodiments, where R^{1b} is unsubstituted or substituted cycloalkyl, R^{1b} is unsubstituted or substituted C₃ cycloalkyl (e.g., cyclopropyl). In several embodiments, where R^{1b} is unsubstituted or substituted cycloalkyl, R^{1b} is unsubstituted or substituted C₄ cycloalkyl (e.g., cyclobutyl). In several embodiments, where R^{1b} is unsubstituted or substituted cycloalkyl, R^{1b} is unsubstituted or substituted C₅ cycloalkyl (e.g., cyclopentyl). In several embodiments, where R^{1b} is unsubstituted or substituted cycloalkyl, R^{1b} is unsubstituted or substituted C₆ cycloalkyl. In several embodiments, where R^{1b} is unsubstituted or substituted heterocyclyl, R^{1b} is unsubstituted or substituted heterocyclyl having 4 to 6-ring members. In several embodiments, where R^{1b} is unsubstituted or substituted heterocyclyl, R^{1b} is unsubstituted or substituted heterocyclyl having 3 to 5-ring members. In several embodiments, where R^{1b} is unsubstituted or substituted heterocyclyl, R^{1b} is unsubstituted or substituted heterocyclyl having 3-ring members. In several embodiments, where R^{1b} is unsubstituted or substituted heterocyclyl, R^{1b} is unsubstituted or substituted heterocyclyl having 4-ring members (e.g., oxetanyl). In several embodiments, where R^{1b} is unsubstituted or substituted heterocyclyl, R^{1b} is unsubstituted or substituted heterocyclyl having 5-ring members. In several embodiments, where R^{1b} is unsubstituted or substituted heterocyclyl, R^{1b} is unsubstituted or substituted heterocyclyl having 6-ring members. In several embodiments, R^{1b} is tetrahydropyranyl. In several embodiments, R^{1b} is tetrahydrofuranyl. In several embodiments, R^{1b} is piperidinyl. In several embodiments, R^{1b} is pyrrolidinyl. In several embodiments, R^{1b} is azetidinyl. In several embodiments, R^{1b} is:

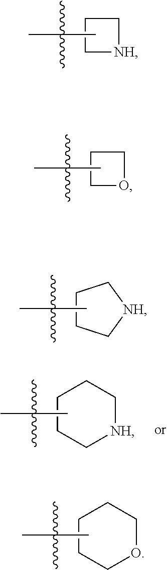

Provided herein as Embodiment 48 is the compound or salt of any one of Embodiments 1 to 47, wherein R^{1b} is an unsubstituted or substituted cycloalkyl spiro ring system or an unsubstituted or substituted heterocyclyl spiro ring system. For example, in several embodiments, R^{1b} is an unsubstituted or substituted cycloalkyl spiro ring system. In several embodiments, R^{1b} is an unsubstituted or substituted heterocyclyl spiro ring system. In several embodiments, R^{1b} is unsubstituted or substituted spiro[3.3]heptanyl. In several embodiments, R^{1b} is unsubstituted or substituted spiro[3.4]octanyl. In several embodiments, R^{1b} is unsubstituted or substituted spiro[3.5]nonanyl. In several embodiments, R^{1b} is unsubstituted or substituted oxaspiro[3.3]heptanyl.

Provided herein as Embodiment 49 is the compound or salt of any one of Embodiments 1 to 47, wherein R^{1b} is an unsubstituted or substituted bridged cycloalkyl ring system or unsubstituted or substituted bridged heterocyclyl ring system. For example, in several embodiments, R^{1b} is an unsubstituted or substituted bridged cycloalkyl ring system. In several embodiments, R^{1b} is an unsubstituted or substituted bridged heterocyclyl ring system.

Provided herein as Embodiment 50 is the compound or salt of any one of Embodiments 1 to 49, wherein R^{1b} is a bicyclic ring system.

Provided herein as Embodiment 51 is the compound or salt of any one of Embodiments 1 to 50, wherein R^{1b} is unsubstituted or substituted heterocyclyl having 1, 2, or 3 heteroatom ring members, each of which independently is nitrogen, oxygen, or —S(O)ₓ—, where x is 0, 1, or 2. For example, in several embodiments, the R^{1b} heterocyclyl includes 1, 2, or 3 heteroatom ring members, each of which independently is nitrogen, oxygen, or —S(O)ₓ—, where x is 0, 1, or 2. In several embodiments, the R^{1b}heterocyclyl includes 1 or 3 heteroatom ring members. In several embodiments, the R^{1b} heterocyclyl includes 2 or 3 heteroatom ring members. In several embodiments, x is 0. In several embodiments, x is 1. In several embodiments, x is 2.

Provided herein as Embodiment 52 is the compound or salt of any one of Embodiments 1 to 51, wherein R^{1b} is unsubstituted or substituted heterocyclyl having 1, 2, or 3 heteroatom ring members, each of which independently is nitrogen or oxygen. For example, in several embodiments, where there are multiple heteroatom ring members, the heteroatom ring members may each be oxygen or there may be a combination of oxygen and nitrogen. In several embodiments, where there are multiple heteroatom ring members, the heteroatom ring members may each be nitrogen or there may be a combination of oxygen and nitrogen. In several embodiments, where there is one heteroatom, that heteroatom may be oxygen. In several embodiments, where there is one heteroatom, that heteroatom may be nitrogen.

Provided herein as Embodiment 53 is the compound or salt of any one of Embodiments 1 to 52, wherein R^{1b} is heterocyclyl having 1 or 2 heteroatom ring members.

Provided herein as Embodiment 54 is the compound or salt of any one of Embodiments 1 to 53, wherein R^{1b} is heterocyclyl having 1 heteroatom ring member.

Provided herein as Embodiment 55 is the compound or salt of any one of Embodiments 1 to 54, wherein R^{1b} is heterocyclyl and each heteroatom ring member of R^{1b} is nitrogen.

Provided herein as Embodiment 56 is the compound or salt of any one of Embodiments 1 to 54, wherein R^{1b} is heterocyclyl and each heteroatom ring member of R^{1b} is oxygen.

Provided herein as Embodiment 57 is the compound or salt of any one of Embodiments 1 to 56, wherein R^{1b} is unsubstituted or substituted heterocyclyl having 3- to 6-ring members. For example, in several embodiments, R^{1b} is unsubstituted or substituted heterocyclyl having 3- to 5-ring members. In several embodiments, R^{1b} is unsubstituted or substituted heterocyclyl having 3- to 4-ring members. In several embodiments, R^{1b} is unsubstituted or substituted heterocyclyl having 4- to 6-ring members. In several embodiments, R^{1b} is unsubstituted or substituted heterocyclyl having 4- to 5-ring members. In several embodiments, R^{1b} is unsubstituted or substituted heterocyclyl having 5- to 6-ring members. In several embodiments, R^{1b} is unsubstituted or substituted heterocyclyl having 3-ring members. In several embodiments, R^{1b} is unsubstituted or substituted heterocyclyl having 4-ring members. In several embodiments, R^{1b} is unsubstituted or substituted heterocyclyl having 5-ring members. In several embodiments, R^{1b} is unsubstituted or substituted heterocyclyl having 6-ring members. In several embodiments, R^{1b} is an unsubstituted heterocyclyl. In several embodiments, R^{1b} is a substituted heterocyclyl.

Provided herein as Embodiment 58 is the compound or salt of any one of any one of Embodiments 1 to 47, wherein R^{1b} is unsubstituted or substituted C₁₋₃ alkyl. For example, in several embodiments, R^{1b} is unsubstituted or substituted C₁₋₂ alkyl. In several embodiments, R^{1b} is unsubstituted or substituted C₂₋₃ alkyl. In several embodiments, R^{1b} is unsubstituted or substituted C₁ alkyl. In several embodiments, R^{1b} is unsubstituted or substituted C₂ alkyl. In several embodiments, R^{1b} is unsubstituted or substituted C₃ alkyl. In several embodiments, the R^{1b} alkyl is unsubstituted. In several embodiments, the R^{1b} alkyl is substituted.

Provided herein as Embodiment 59 is the compound or salt of any one of Embodiments 1 to 47, wherein R^{1b} is unsubstituted or substituted C₁₋₃ haloalkyl. For example, in several embodiments, R^{1b} is unsubstituted or substituted C₁₋₂ haloalkyl. In several embodiments, R^{1b} is unsubstituted or substituted C₂-3 haloalkyl. In several embodiments, R^{1b} is unsubstituted or substituted C₁ haloalkyl. In several embodiments, R^{1b} is unsubstituted or substituted C₂haloalkyl. In several embodiments, R^{1b} is unsubstituted or substituted C₃ haloalkyl. In several embodiments, the R^{1b} haloalkyl is unsubstituted. In several embodiments, the R^{1b} haloalkyl is substituted. In several embodiments, the R^{1b} haloalkyl is perhalogenated (e.g., perfluorinated). In several embodiments, where R^{1b} is C₁₋₃ haloalkyl, the haloalkyl can include 1, 2, 3, 4, 5, 6, or 7 halogen atoms (e.g., —F).

Provided herein as Embodiment 60 is the compound or salt of any one of Embodiments 1 to 47, wherein R^{1b} is unsubstituted or substituted C₃₋₆ cycloalkyl. For example, in several embodiments, R^{1b} is unsubstituted or substituted C₃₋ₛ cycloalkyl. In several embodiments, R^{1b} is unsubstituted or substituted C₃₋₄ cycloalkyl. In several embodiments, R^{1b} is unsubstituted or substituted C₄₋₆ cycloalkyl. In several embodiments, R^{1b} is unsubstituted or substituted C₅₋₆ cycloalkyl. In several embodiments, R^{1b} is unsubstituted or substituted C₃ cycloalkyl. In several embodiments, R^{1b} is unsubstituted or substituted C₄ cycloalkyl. In several embodiments, R^{1b} is unsubstituted or substituted C₅ cycloalkyl. In several embodiments, R^{1b} is unsubstituted or substituted C₆ cycloalkyl. In several embodiments, the R^{1b}cycloalkyl is unsubstituted. In several embodiments, the R^{1b} haloalkyl is cycloalkyl.

Provided herein as Embodiment 61 is the compound or salt of any one of Embodiments 1 to 46, wherein R^{1b} is unsubstituted or substituted —(C₁₋₆ alkylene)-O-(C₁₋₆ alkyl). For example, in several embodiments, R^{1b} is unsubstituted or substituted —(C₁₋₆ alkylene)-O-(C₁ alkyl) (e.g., —CH₂CH₂OCH₃, —CH₂OCH₃, etc.). In several embodiments, R^{1b} is unsubstituted or substituted —(C₁₋₆ alkylene)-O-(C₂ alkyl). In several embodiments, R^{1b} is unsubstituted or substituted —(C₁₋₆ alkylene)-O-(C₃ alkyl). In several embodiments, R^{1b} is unsubstituted or substituted —(C₁₋₆ alkylene)-O-(C₄ alkyl). In several embodiments, R^{1b} is unsubstituted or substituted —(C₁₋₆ alkylene)-O-(C₅ alkyl). In several embodiments, R^{1b} is unsubstituted or substituted —(C₁₋₆ alkylene)-O-(C₆ alkyl). In several embodiments, R^{1b} is unsubstituted or substituted —(C₁₋₆ alkylene)-O-(C₁₋₂ alkyl). In several embodiments, R^{1b} is unsubstituted or substituted —(C₁₋₆ alkylene)-O—(C₁₋₃ alkyl). In several embodiments, as disclosed elsewhere herein, the R^{1b} alkylene is C₁₋₂ alkylene. In several embodiments, the R^{1b} alkylene is C₂₋₃ alkylene. In several embodiments, the R^{1b} alkylene is C₁₋₃ alkylene. In several embodiments, the R^{1b} alkylene is C₂₋₄ alkylene. In several embodiments, the R^{1b} alkylene is C₂₋₅ alkylene. In several embodiments, the R^{1b} alkylene is C₁ alkylene. In several embodiments, the R^{1b} alkylene is C₂ alkylene. In several embodiments, the R^{1b}alkylene is C₃ alkylene. In several embodiments, the R^{1b} alkylene is C₄ alkylene. In several embodiments, the R^{1b} alkylene is C₅ alkylene. In several embodiments, R^{1b} is unsubstituted or substituted —(C₁₋₃ alkylene)-O—(C₁₋₃ alkyl). In several embodiments, R^{1b} is unsubstituted or substituted —(C₁₋₂ alkylene)-O-(C₁₋₂ alkyl). In several embodiments, R^{1b} is unsubstituted or substituted —(C₁₋₃ alkylene)-O-(C₁₋₂ alkyl). In several embodiments, R^{1b} is unsubstituted or substituted —(C₁₋₃ alkylene)-OMe. In several embodiments, the alkylene-O-alkyl is unsubstituted. In several embodiments, the alkylene-O-alkyl is substituted. In several embodiments, R^{1b} is —CH₂—O—CH₃. In several embodiments, R^{1b} may be represented by one of the following structures:

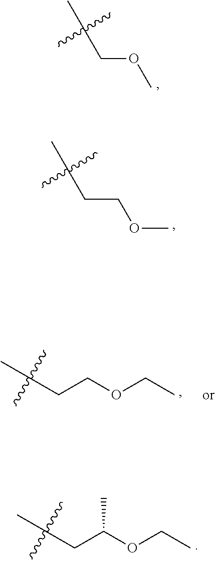

In several embodiments, where R^{1b} is —(C₁₋₃ alkylene)-O-(C₁₋₃ alkyl) and wherein R^{1b} is substituted with 2 or 3 substituents (e.g., —F), then R^{1b} may be

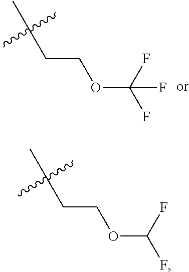 [SMILES: *CCOC(F)(F)F ; *CCOC(F)F]

respectively.

Provided herein as Embodiment 62 is the compound or salt of any one of Embodiments 1 to 46, wherein R^{1b} is unsubstituted or substituted —(C₁₋₃alkylene)-O-(C₁₋₃ alkyl).

Provided herein as Embodiment 63 is the compound or salt of any one of Embodiments 1 to 62, wherein R^{1b} is substituted with 1, 2, 3, or 4 substituents. For example, in several embodiments, R^{1b} is substituted with 1 to 4 substituents. In several embodiments, R^{1b} is substituted with 1 to 3 substituents. In several embodiments, R^{1b} is substituted with 1 to 2 substituents. In several embodiments, R^{1b} is substituted with 2 to 4 substituents. In several embodiments, R^{1b} is substituted with 3 to 4 substituents. In several embodiments, R^{1b} is substituted with 2 to 3 substituents.

Provided herein as Embodiment 64 is the compound or salt of any one of Embodiments 1 to 62, wherein R^{1b} is substituted with 1, 2, or 3 substituents.

Provided herein as Embodiment 65 is the compound or salt of any one of Embodiments 1 to 62, wherein R^{1b} is substituted with 1 or 2 substituents.

Provided herein as Embodiment 66 is the compound or salt of any one of Embodiments 1 to 62, wherein R^{1b} is substituted with 1 substituent.

Provided herein as Embodiment 67 is the compound or salt of any one of Embodiments 1 to 66, wherein, when R^{1b} is substituted, each R^{1b} substituent independently is halogen, unsubstituted or substituted C₁₋₃alkyl, or unsubstituted or substituted C₁₋₃alkoxy. For example, in several embodiments, each R^{1b} substituent independently is halogen, unsubstituted C₁₋₃alkyl, or unsubstituted C₁₋₃alkoxy. In several embodiments, each R^{1b} substituent independently is halogen, substituted C₁₋₃alkyl, or substituted C₁₋₃alkoxy. In several embodiments, each R^{1b} substituent independently is halogen or C₁₋₃alkoxy. In several embodiments, each R^{1b} substituent independently is halogen or C₁₋₃alkyl. In several embodiments, each R^{1b} substituent independently is C₁₋₃alkyl or C₁₋₃alkoxy. In several embodiments, each R^{1b} substituent independently is —F, —Cl, or —Br. In several embodiments, each R^{1b} substituent independently is —F or —Cl. In several embodiments, R^{1b} is substituted with —F. In several embodiments, R^{1b} is substituted with C₁₋₂ alkyl. In several embodiments, R^{1b} is substituted with C₂, alkyl. In several embodiments, R^{1b} is substituted with methyl. In several embodiments, R^{1b} is substituted with C₂ alkyl. In several embodiments, R^{1b} is substituted with C₃ alkyl. In several embodiments, R^{1b} is substituted with C₁₋₂ alkoxy. In several embodiments, R^{1b} is substituted with C₂₋₃ alkoxy. In several embodiments, R^{1b} is substituted with C₁ alkoxy. In several embodiments, R^{1b} is substituted with C₂ alkoxy. In several embodiments, R^{1b} is substituted with C₃ alkoxy. In several embodiments, where R^{1b} is substituted with unsubstituted or substituted C₁₋₃alkyl or unsubstituted or substituted C₁₋₃alkoxy, each of the C₁₋₃alkyl or C₁₋₃alkoxy may be unsubstituted or substituted with 1 to 9 halogen groups or C₁₋₃ alkoxy. In several embodiments, where R^{1b} is substituted with unsubstituted or substituted C₁₋₃alkyl or unsubstituted or substituted C₁₋₃alkoxy, each of the C₁₋₃alkyl or C₁₋₃alkoxy may be unsubstituted or substituted with 1 to 9—F groups or C₁₋₃ alkoxy. In several embodiments, where R^{1b} is substituted with one or more substituents, each of the substituents independently is:

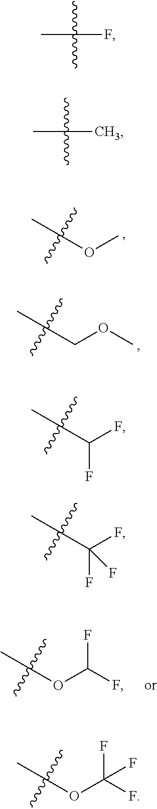

In several embodiments, R^{1b} together with its substituents is difluorocyclobutyl (e.g., 3,3-diflourocyclobutyl) or difluorocyclohexyl (e.g., 4,4-difluorocyclohexyl). In several embodiments, R^{1b}together with its substituents is N-methylpiperidinyl. In several embodiments, R^{1b} together with its substituents is N-methylpyrrolidinyl. In several embodiments, R^{1b} together with its substituents is N-methylazetidinyl. In several embodiments, R^{1b} together with its substituents, where present, is

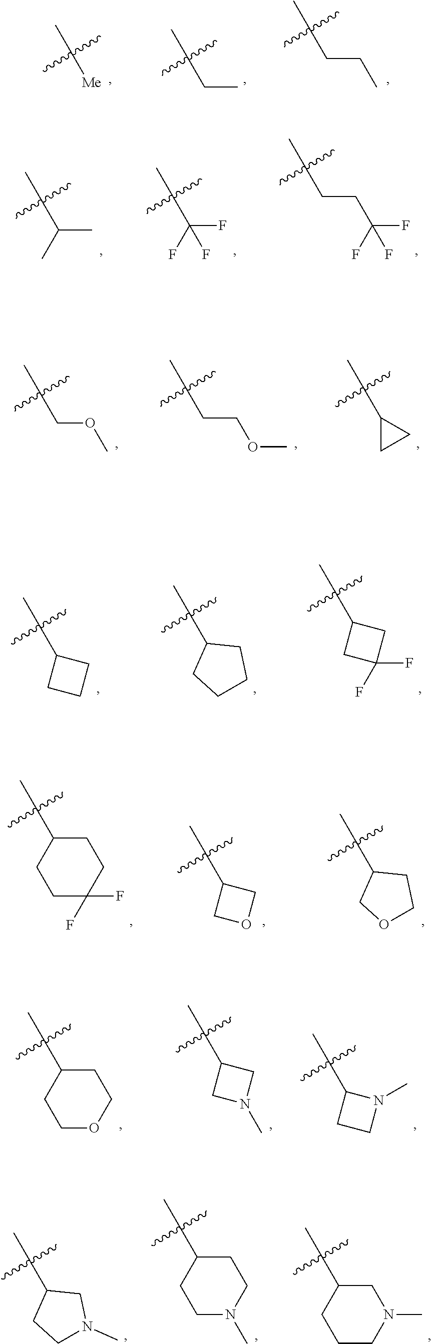

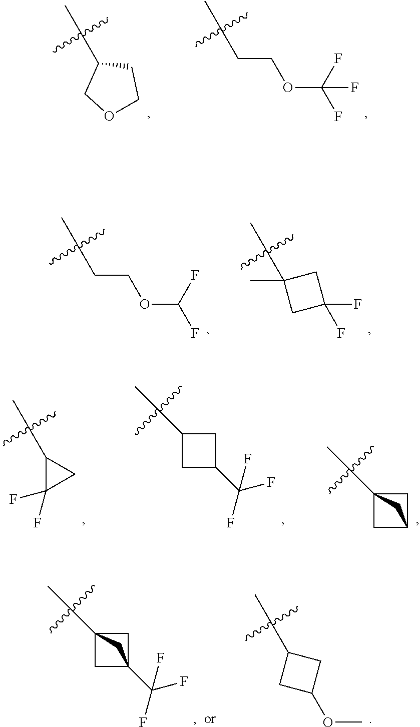

Provided herein as Embodiment 68 is the compound or salt of any one of Embodiments 1 to 67, wherein, when R^{1b} is substituted, each R^{1b} substituent independently is —F, —OH, methyl, or —OMe. For example, in several embodiments, each R^{1b} substituent independently is —OH, methyl, or —OMe. In several embodiments, each R^{1b} substituent independently is —F, methyl, or —OMe. In several embodiments, each R^{1b} substituent independently is —F, —OH, or —OMe. In several embodiments, each R^{1b}substituent independently is —F, —OH, or methyl. In several embodiments, each R^{1b} substituent independently is —F or -OH. In several embodiments, each R^{1b} substituent independently is —F or methyl. In several embodiments, each R^{1b} substituent independently is —F or —OMe. In several embodiments, each R^{1b} substituent independently is —OH or methyl. In several embodiments, each R^{1b} substituent independently is —OH or —OMe. In several embodiments, each R^{1b} substituent independently is methyl or —OMe.

Provided herein as Embodiment 69 is the compound or salt of any one of Embodiments 1 to 62, wherein R^{1b} is unsubstituted.

Provided herein as Embodiment 70 is the compound or salt of any one of Embodiments 1 to 46, wherein R^{1b} is —H.

Provided herein as Embodiment 71 is the compound or salt of any one of Embodiments 1 to 33, wherein R^{1a} and R^{1b} together form an unsubstituted or substituted C₃₋₁₀ cycloalkyl or an unsubstituted or substituted heterocyclyl having 3- to 10-ring members. For example, in several embodiments, R^{1a} and R^{1b} together form a C₄₋₆ cycloalkyl. In several embodiments, R^{1a} and R^{1b} together form a C₃₋₅ cycloalkyl. In several embodiments, R^{1a} and R^{1b} together form a C₄₋₆ cycloalkyl. In several embodiments, R^{1a} and R^{1b}together form a C₃₋₄ cycloalkyl. In several embodiments, R^{1a} and R^{1b} together form a C₅₋₆ cycloalkyl. In several embodiments, R^{1a} and R^{1b} together form a C₈₋₁₀ cycloalkyl. In several embodiments, R^{1a} and R^{1b}together form a C₃ cycloalkyl (e.g., cyclopropyl). In several embodiments, R^{1a} and R^{1b} together form a C₄ cycloalkyl (e.g., cyclobutyl). In several embodiments, R^{1a} and R^{1b} together form a C₅ cycloalkyl (e.g., cyclopentyl, bicyclo[1.1.1]pentan-1-yl, etc.). In several embodiments, R^{1a} and R^{1b} together form a C₆ cycloalkyl (e.g., cyclohexyl, spiro[2.3]hexyl, etc.). In several embodiments, R^{1a} and R^{1b} together form a C₇ cycloalkyl (e.g., cycloheptyl, spiro[3.3]heptyl, etc.). In several embodiments, R^{1a} and R^{1b} together form a C₅ cycloalkyl (e.g., spiro[3.4]octyl, etc.). In several embodiments, R^{1a} and R^{1b} together form a C₉ cycloalkyl (e.g., spiro[3.5]nonyl, etc.). In several embodiments, R^{1a} and R^{1b} together form a heterocyclyl having 4 to 6-ring members. In several embodiments, R^{1a} and R^{1b} together form a heterocyclyl having 5 to 6-ring members. In several embodiments, R^{1a} and R^{1b} together form a heterocyclyl having 3 to 5-ring members. In several embodiments, R^{1a} and R^{1b} together form a heterocyclyl having 3 to 4-ring members. In several embodiments, R^{1a} and R^{1b} together form a heterocyclyl having 4 to 5-ring members. In several embodiments, R^{1a} and R^{1b} together form a heterocyclyl having 3-ring members. In several embodiments, R^{1a} and R^{1b} together form a heterocyclyl having 4-ring members (e.g., azetidinyl, oxetanyl, etc.). In several embodiments, R^{1a} and R^{1b} together form a heterocyclyl having 5-ring members (e.g., pyrrolidinyl, tetrahydrofuranyl, etc.). In several embodiments, R^{1a} and R^{1b} together form a heterocyclyl having 6-ring members (e.g., piperidinyl, tetrahydropyranyl, tetrahydro-2H-thiopyran-1,1-dioxide, etc.). In several embodiments, R^{1a} and R^{1b} together form a heterocyclyl having 7-ring members (e.g., oxaspiro[3.3]heptanyl, etc.). In several embodiments, R^{1a} and R^{1b} together form an unsubstituted or substituted C₃₋₉ cycloalkyl or an unsubstituted or substituted heterocyclyl having 3- to 9-ring members. In several embodiments, R^{1a} and R^{1b} together form an unsubstituted or substituted C₃₋₉ cycloalkyl or an unsubstituted or substituted heterocyclyl having 4- to 8-ring members. In several embodiments, R^{1a} and R^{1b} together form an unsubstituted or substituted C₃₋₉ cycloalkyl or an unsubstituted or substituted heterocyclyl having 4- to 7-ring members. In several embodiments, when R^{1a} and R^{1b} together form a cycloalkyl or heterocyclyl that is unsubstituted, then

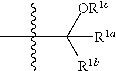

may be represented as:

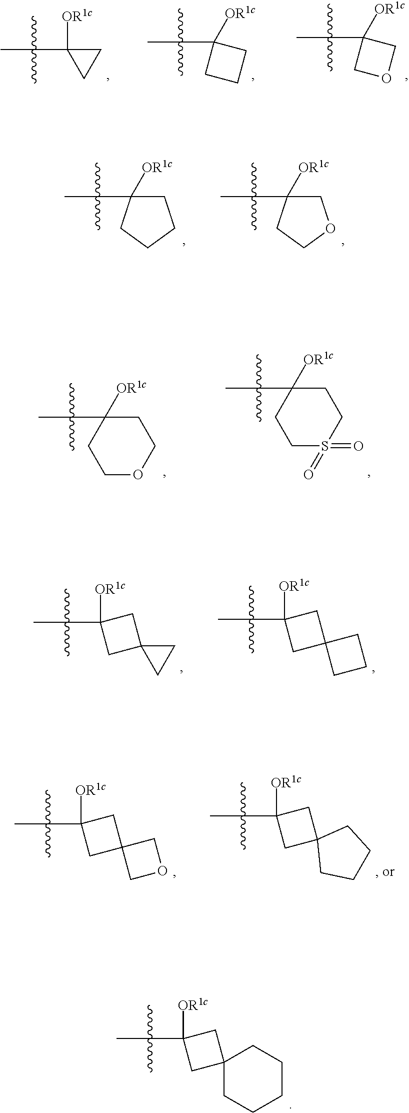

In several embodiments, when R^{1a} and R^{1b} together form a cycloalkyl or heterocyclyl that is substituted, then

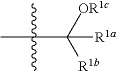

may be represented as:

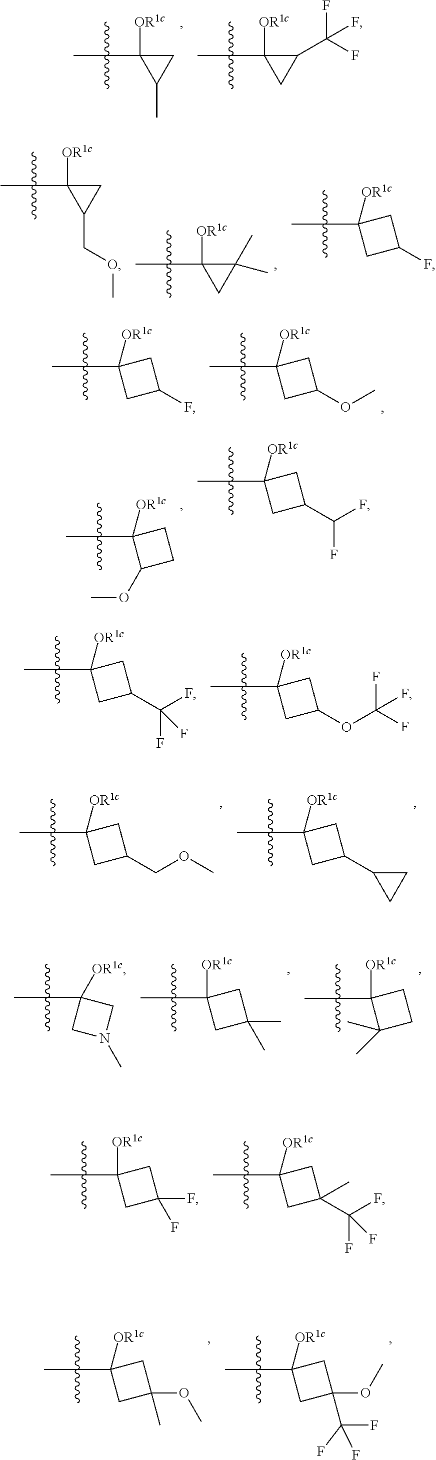

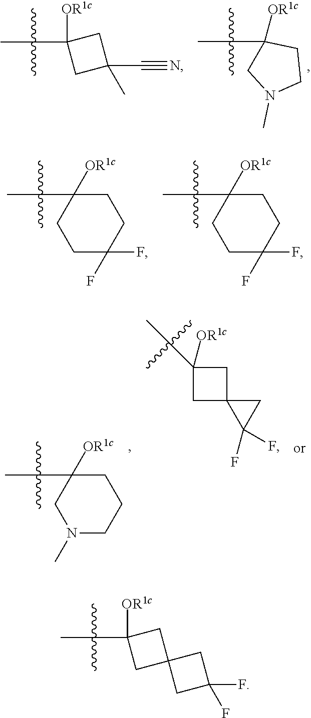

In several embodiments, where R^{1c} is H and when R^{1a} and R^{1b} together form a cycloalkyl or heterocyclyl that is unsubstituted,

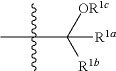

may be represented as:

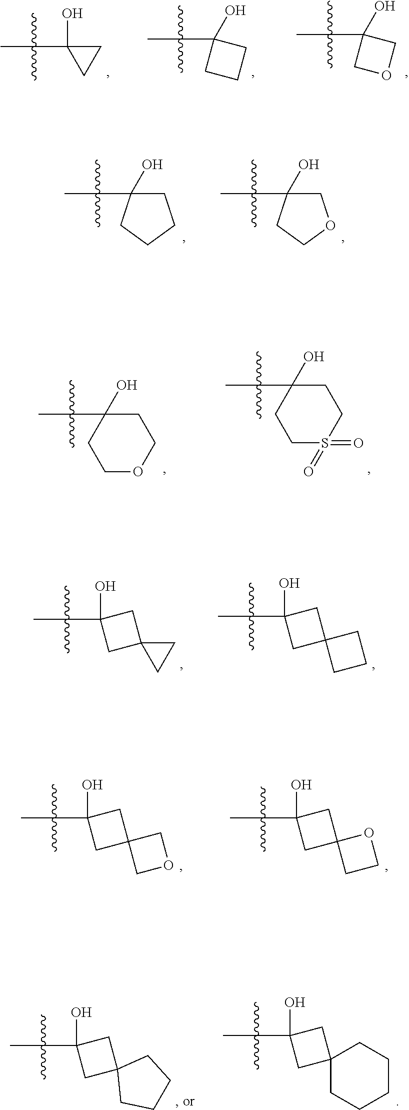

In several embodiments, where R^{1c} is H and when R^{1a} and R^{1b} together form a cycloalkyl or heterocyclyl that is substituted,

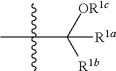

may be represented as;

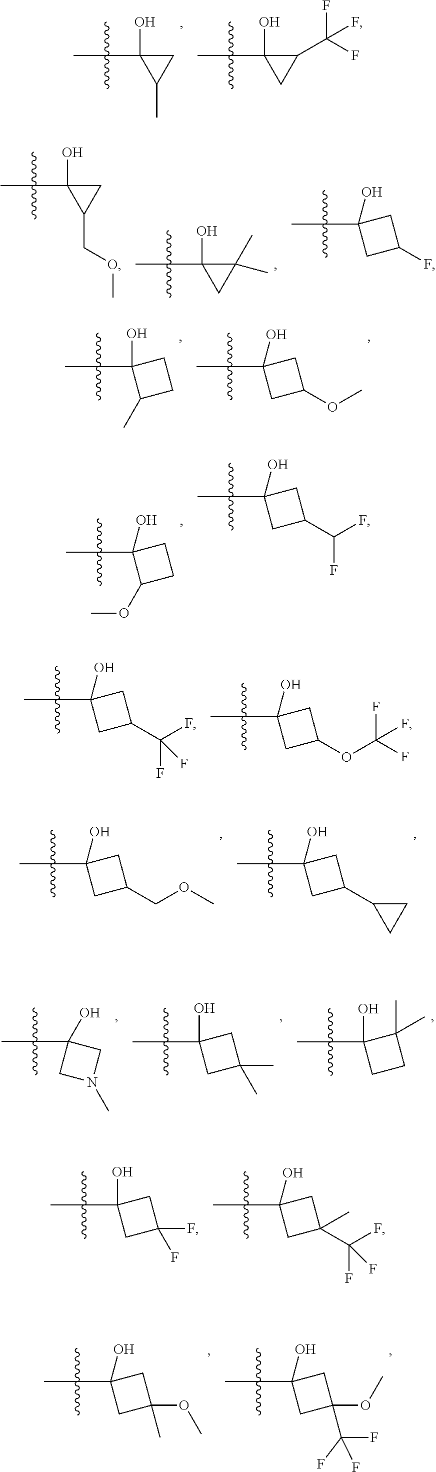

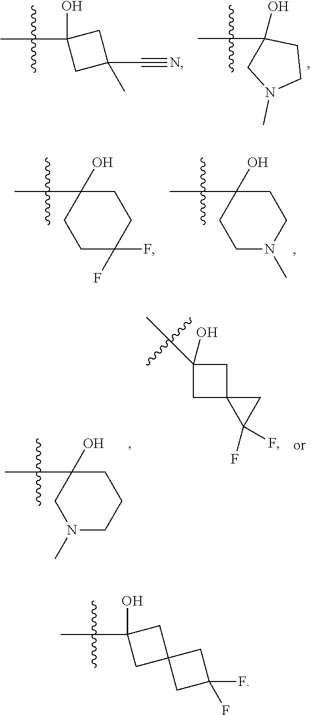

In several embodiments, a substituent on a cycloalkyl or heterocyclyl formed by R^{1a} and R^{1b} may be cis to the —OR^{1c} group. In several embodiments, a substituent on a cycloalkyl or heterocyclyl formed by R^{1a} and R^{1b} may be trans to the —OR^{1c} group. In several embodiments, where more than one substituent is provided on a cycloalkyl or heterocyclyl formed by R^{1a} and R^{1b}, each of the substituents may independently be either cis or trans to the —OR^{1c} group. For example, in this structure, the —CH₃ and the —OH are trans to one another and the —CF₃ and —OH are cis to one another:

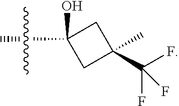

Provided herein as Embodiment 72 is the compound or salt of any one of Embodiments 1 to 33, wherein R^{1a} and R^{1b} together form an unsubstituted or substituted heterocyclyl having 3- to 10-ring members. For example, in several embodiments, R^{1a} and R^{1b} together form an unsubstituted or substituted 3-membered heterocyclyl (e.g., an unsubstituted or substituted heterocyclyl having 3 ring members). In several embodiments, R^{1a} and R^{1b} together form an unsubstituted or substituted 4-membered heterocyclyl (e.g., an unsubstituted or substituted heterocyclyl having 4 ring members). In several embodiments, R^{1a} and R^{1b} together form an unsubstituted or substituted 5-membered heterocyclyl (e.g., an unsubstituted or substituted heterocyclyl having 5 ring members). In several embodiments, R^{1a} and R^{1b} together form an unsubstituted or substituted 6-membered heterocyclyl (e.g., an unsubstituted or substituted heterocyclyl having 6 ring members). In several embodiments, R^{1a} and R^{1b} together form an unsubstituted or substituted 7-membered heterocyclyl (e.g., an unsubstituted or substituted heterocyclyl having 7 ring members). In several embodiments, R^{1a} and R^{1b} together form an unsubstituted or substituted 8-membered heterocyclyl (e.g., an unsubstituted or substituted heterocyclyl having 8 ring members). In several embodiments, R^{1a} and R^{1b} together form an unsubstituted or substituted 9-membered heterocyclyl (e.g., an unsubstituted or substituted heterocyclyl having 9 ring members). In several embodiments, R^{1a} and R^{1b} together form an unsubstituted or substituted 10-membered heterocyclyl (e.g., an unsubstituted or substituted heterocyclyl having 10 ring members).

Provided herein as Embodiment 73 is the compound or salt of any one of Embodiments 1 to 33, wherein R^{1a} and R^{1b} together form an unsubstituted or substituted heterocyclyl having 5- to 10-ring members.

Provided herein as Embodiment 74 is the compound or salt of any one of Embodiments 1 to 33, wherein R^{1a} and R^{1b} together form an unsubstituted or substituted heterocyclyl having 7- to 10-ring members. For example, in several embodiments, R^{1a} and R^{1b} together form an unsubstituted or substituted 7-membered heterocyclyl. In several embodiments, R^{1a} and R^{1b} together form an unsubstituted or substituted 8-membered heterocyclyl. In several embodiments, R^{1a} and R^{1b} together form an unsubstituted or substituted 9-membered heterocyclyl. In several embodiments, R^{1a} and R^{1b} together form an unsubstituted or substituted 10-membered heterocyclyl. In several embodiments, the heterocyclyl may be spiro cyclic. In several embodiments, the heterocyclyl may be a bridged multicyclic ring.

Provided herein as Embodiment 75 is the compound or salt of any one of Embodiments 1 to 33, wherein R^{1a} and R^{1b} together form an unsubstituted or substituted heterocyclyl having 4- to 6-ring members. For example, in several embodiments, R^{1a} and R^{1b} together form an unsubstituted or substituted 4-membered heterocyclyl (e.g., a heterocyclyl having 4 ring members). In several embodiments, R^{1a} and R^{1b} together form an unsubstituted or substituted 5-membered heterocyclyl (e.g., a heterocyclyl having 5 ring members). In several embodiments, R^{1a} and R^{1b} together form an unsubstituted or substituted 6-membered heterocyclyl (e.g., a heterocyclyl having 6 ring members, such as tetrahydropyranyl, etc.).

Provided herein as Embodiment 76 is the compound or salt of any one of Embodiments 1 to 33 and 71 to 75, wherein, when R^{1a} and R^{1b} together form an unsubstituted or substituted heterocyclyl, the heterocyclyl includes 1, 2, or 3 heteroatom ring members, each of which independently is nitrogen, oxygen, or —S(O)ₓ—, where x is 0, 1, or 2. For example, in several embodiments, R^{1a} and R^{1b} together form a heterocyclyl having 1 or 3 heteroatom ring members. In several embodiments, R^{1a} and R^{1b} together form a heterocyclyl having 2 or 3 heteroatom ring members. In several embodiments, R^{1a} and R^{1b}together form a heterocyclyl having 1 or 2 heteroatom ring members. In several embodiments, R^{1a} and R^{1b} together form a heterocyclyl having 1 or 3 heteroatom ring members. In several embodiments, R^{1a} and R^{1b} together form a heterocyclyl having S(O)₂ as a ring member. In several embodiments, R^{1a} and R^{1b} together form a heterocyclyl having N as a ring member. In several embodiments, R^{1a} and R^{1b}together form a heterocyclyl having two N ring members. In several embodiments, R^{1a} and R^{1b} together form a heterocyclyl having three N ring members. In several embodiments, R^{1a} and R^{1b} together form a heterocyclyl having O as a ring member. In several embodiments, R^{1a} and R^{1b} together form a heterocyclyl having S as a ring member. In several embodiments, x is 0. In several embodiments, x is 1. In several embodiments, x is 2.

Provided herein as Embodiment 77 is the compound or salt of any one of Embodiments 1 to 33 and 71 to 75, wherein, when R^{1a} and R^{1b} together form an unsubstituted or substituted heterocyclyl, the heterocyclyl includes 1, 2, or 3 heteroatom ring members, each of which independently is nitrogen or oxygen.

Provided herein as Embodiment 78 is the compound or salt of any one of Embodiments 1 to 33 and 71 to 77, wherein, when R^{1a} and R^{1b} together form an unsubstituted or substituted heterocyclyl, the heterocyclyl includes 1 or 2 heteroatom ring members.

Provided herein as Embodiment 79 is the compound or salt of any one of Embodiments 1 to 33 and 71 to 78, wherein, when R^{1a} and R^{1b} together form an unsubstituted or substituted heterocyclyl, the heterocyclyl includes 1 heteroatom ring member.

Provided herein as Embodiment 80 is the compound or salt of any one of Embodiments 1 to 33 and 71 to 79, wherein R^{1a} and R^{1b} together form an unsubstituted or substituted heterocyclyl the heteroatom ring member or ring members of R^{1a} and R^{1b} comprise or consist of nitrogen.

Provided herein as Embodiment 81 is the compound or salt of any one of Embodiments 1 to 33 and 71 to 80, wherein R^{1a} and R^{1b} together form an unsubstituted or substituted heterocyclyl and the heteroatom ring member or heteroatom ring members of R^{1a} and R^{1b} comprise or consist of oxygen.

Provided herein as Embodiment 82 is the compound or salt of any one of Embodiments 1 to 33 and 71, wherein R^{1a} and R^{1b} together form an unsubstituted or substituted C₃₋₁₀ cycloalkyl. For example, in several embodiments, R^{1a} and R^{1b} together form a C₈₋₁₀ cycloalkyl. In several embodiments, R^{1a} and R^{1b} together form a C₇₋₁₀ cycloalkyl. In several embodiments, R^{1a} and R^{1b} together form a C₃₋₆ cycloalkyl. In several embodiments, the cycloalkyl may be spiro cyclic. In several embodiments, the cycloalkyl may be a bridged multicyclic ring.

Provided herein as Embodiment 83 is the compound or salt of any one of Embodiments 1 to 33 and 71, wherein R^{1a} and R^{1b} together form an unsubstituted or substituted C₅₋₁₀ cycloalkyl.

Provided herein as Embodiment 84 is the compound or salt of any one of Embodiments 1 to 33 and 71, wherein R^{1a} and R^{1b} together form an unsubstituted or substituted C₇₋₁₀ cycloalkyl.

Provided herein as Embodiment 85 is the compound or salt of any one of Embodiments 1 to 33 and 71, wherein R^{1a} and R^{1b} together form an unsubstituted or substituted C₃₋₆ cycloalkyl. For example, in several embodiments, R^{1a} and R^{1b} together form an unsubstituted or substituted cyclopropyl. In several embodiments, R^{1a} and R^{1b} together form an unsubstituted or substituted cyclobutyl. In several embodiments, R^{1a} and R^{1b} together form an unsubstituted or substituted cyclopentyl. In several embodiments, R^{1a} and R^{1b} together form an unsubstituted or substituted cyclohexyl.

Provided herein as Embodiment 86 is the compound or salt of any one of Embodiments 1 to 33 and 71 to 85, wherein R^{1a} and R^{1b} together form a spiro ring system. For example, in several embodiments, when R^{1c} is

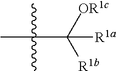

may be represented as:

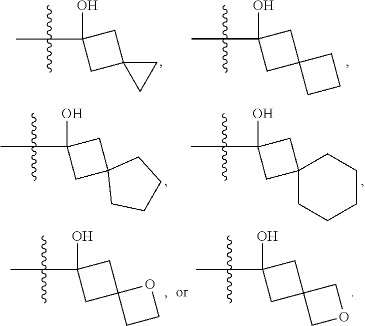

In several embodiments, when R^{1a} and R^{1b} together form a spiro ring system that is substituted and R^{1c} is

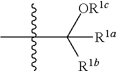

may be represented as:

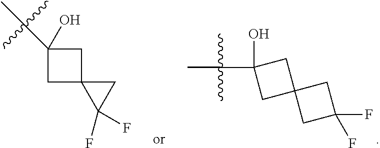 [SMILES: *C1(O)CC2(C1)CC2(F)F ; *C1(O)CC2(C1)CC(F)(F)C2]

Provided herein as Embodiment 87 is the compound or salt of any one of Embodiments 1 to 33 and 71 to 86, wherein R^{1a} and R^{1b} together form a bicyclic ring system.

Provided herein as Embodiment 88 is the compound or salt of any one of Embodiments 1 to 33 and 71 to 87, wherein R^{1a} and R^{1b} together form a cycle that is substituted with 1, 2, 3, or 4 substituents.

Provided herein as Embodiment 89 is the compound or salt of any one of Embodiments 1 to 33 and 71 to 87, wherein R^{1a} and R^{1b} together form a cycle that is substituted with 1, 2, or 3 substituents. For example, in several embodiments, the R^{1a} and R^{1b} cycle is substituted with 2 to 3 substituents. In several embodiments, the R^{1a} and R^{1b} cycle is substituted with 1 or 3 substituents. In several embodiments, the R^{1a} and R^{1b} cycle is substituted with 1 or 2 substituents.

Provided herein as Embodiment 90 is the compound or salt of any one of Embodiments 1 to 33 and 71 to 87, wherein R^{1a} and R^{1b} together form a cycle that is substituted with 1 or 2 substituents.

Provided herein as Embodiment 91 is the compound or salt of any one of Embodiments 1 to 33 and 71 to 87, wherein R^{1a} and R^{1b} together form a cycle that is substituted with 1 substituent.

Provided herein as Embodiment 92 is the compound or salt of any one of Embodiments 1 to 33 and 71 to 91, wherein R^{1a} and R^{1b} together form a cycle that is substituted with -(C₁₋₃ alkylene)-O-(C₁₋₃ alkyl). For example, in several embodiments, wherein R^{1a} and R^{1b} together form a cycle that is substituted with C₁₋₃ alkylene-O—C₁₋₃ alkyl as the heteroalkyl, the C₁₋₃ alkylene-O—C₁₋₃ alkyl may be represented by one of the following structures:

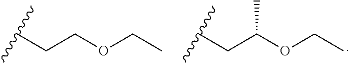

For example, in several embodiments, R^{1a} and R^{1b} together form a cycle that is substituted with alkyl substituted with C₁ alkoxy. In several embodiments, R^{1a} and R^{1b} together form a cycle that is substituted with alkyl substituted with C₂ alkoxy. In several embodiments, R^{1a} and R^{1b} together form a cycle that is substituted with alkyl substituted with C₃ alkoxy. In several embodiments, an alkyl substituent of the R^{1a} and R^{1b} cycle is C₁₋₂ alkyl. In several embodiments, an alkyl substituent of the R^{1a} and R^{1b} cycle is C₁₋₃ alkyl. In several embodiments, an alkyl substituent of the R^{1a} and R^{1b} cycle is methyl. In several embodiments, an alkyl substituent of the R^{1a} and R^{1b} cycle is C₂ alkyl (e.g., ethyl). In several embodiments, an alkyl substituent of the R^{1a} and R^{1b} cycle is C₃ alkyl (e.g., propyl or isopropyl).

Provided herein as Embodiment 93 is the compound or salt of any one of Embodiments 1 to 33 and 71 to 91, wherein R^{1a} and R^{1b} together form a cycle that is substituted and each substituent of the cycle independently is halogen, —OH, oxo, —N(R^{b})₂, unsubstituted or substituted C₁₋₃ alkyl, unsubstituted or substituted C₁₋₃alkoxy, —CN, C₃₋₁₀ cycloalkyl, or heterocyclyl having 3 to 10 ring members and 1, 2, or 3 heteroatom ring members, each of which independently is nitrogen, oxygen, or —S(O)_{W}—, where w is 0, 1, or 2, wherein each of the C₁₋₃ alkyl and C₁₋₃ alkoxy may be unsubstituted or substituted with 1 to 6 halogen groups or C₁₋₃ alkoxy. For example, in several embodiments, the R^{1a} and R^{1b} cycle is substituted with C₃₋₆ cycloalkyl or heterocyclyl having 3 to 10 ring members and 1 or 2 heteroatom ring members. In several embodiments, the substituent is a C₃₋₆ cycloalkyl. In several embodiments, the substituent is a heterocyclyl substituent having 3 to 6 ring members. In several embodiments, the heterocyclyl substituent comprises 1 or 2 heteroatom ring members, each of which independently is nitrogen or oxygen. In several embodiments, the R^{1a} and R^{1b} cycle is substituted with halogen (e.g., —F). In several embodiments, the R^{1a} and R^{1b} cycle is substituted with —OH. In several embodiments, the R^{1a} and R^{1b}cycle is substituted with oxo. In several embodiments, the R^{1a} and R^{1b} cycle is substituted with —N(R^{b})₂. In several embodiments, the R^{1a} and R^{1b} cycle is substituted with C₁₋₃ alkyl. In several embodiments, the R^{1a} and R^{1b} cycle is substituted with -CN. In several embodiments, the R^{1a} and R^{1b} cycle is substituted with C₁₋₃alkoxy. In several embodiments, the R^{1a} and R^{1b} cycle is substituted with C₁₋₃ alkyl or C₁₋₃alkoxy. In several embodiments, the C₁₋₃ alkyl or C₁₋₃alkoxy substituents of the R^{1a} and R^{1b} cycle are further substituted with 1-6 halogens. In several embodiments, a C₁₋₃ alkyl substituent of the R^{1a} and R^{1b}cycle is substituted with 1-6 halogens (e.g., providing a —CH₂F, —CHF₂, or —CF₃ substituent on R^{1a} and R^{1b}cycle, etc.). In several embodiments, the C₁₋₃alkoxy substituent of the R^{1a} and R^{1b} cycle is substituted with 1-6 halogens (e.g., providing a —OCF₃ substituent on R^{1a} and R^{1b} cycle, etc.). In several embodiments, the C₁₋₃ alkyl or C₁₋₃alkoxy substituents of the R^{1a} and R^{1b} cycle are further substituted with 1-3 halogens. In several embodiments, the C₁₋₃ alkyl or C₁₋₃alkoxy substituents of the R^{1a} and R^{1b} cycle are further substituted with 1-6—F atoms. In several embodiments, the C₁₋₃ alkyl or C₁₋₃alkoxy substituents of the R^{1a} and R^{1b} cycle are further substituted with 1-3—F atoms. In several embodiments, a C₁₋₃ alkyl substituent of the R^{1a} and R^{1b} cycle may be further substituted with 1-6 halogens. In several embodiments, a C₁₋₃ alkyl substituent of the R^{1a} and R^{1b} cycle may be further substituted with 1-3 halogens. In several embodiments, a C₁₋₃ alkyl substituent of the R^{1a} and R^{1b} cycle may be further substituted with 1-6—F atoms. In several embodiments, a C₁₋₃ alkyl substituent of the R^{1a} and R^{1b} cycle may be further substituted with 1-3—F atoms. In several embodiments, a C₁₋₃ alkyl substituent of the R^{1a} and R^{1b} cycle may be further substituted with 1, 2, 3, 4, 5, or 6—F atoms. In several embodiments, a C₁₋₃ alkyl substituent of the R^{1a} and R^{1b} cycle may be further substituted with 1, 2, or 3—F atoms. In several embodiments, the R^{1a} and R^{1b} cycle is substituted with a C₁ alkyl substituent and the C₁ alkyl substituent of the R^{1a} and R^{1b} cycle may be further substituted with 1, 2, or 3—F atoms. In several embodiments, a C₁₋₃ alkoxy substituent of the R^{1a} and R^{1b} cycle may be further substituted with 1-6 halogens. In several embodiments, a C₁₋₃ alkoxy substituent of the R^{1a} and R^{1b} cycle may be further substituted with 1-3 halogens. In several embodiments, a C₁₋₃ alkoxy substituent of the R^{1a} and R^{1b} cycle may be further substituted with 1-6—F atoms. In several embodiments, a C₁₋₃ alkoxy substituent of the R^{1a} and R^{1b} cycle may be further substituted with 1-3—F atoms. In several embodiments, a C₁₋₃ alkoxy substituent of the R^{1a} and R^{1b} cycle may be further substituted with 1, 2, 3, 4, 5, or 6—F atoms. In several embodiments, a C₁₋₃ alkoxy substituent of the R^{1a} and R^{1b} cycle may be further substituted with 1, 2, or 3—F atoms. In several embodiments, the R^{1a} and R^{1b} cycle is substituted with a C₁ alkoxy substituent and the C₁ alkoxy substituent of the R^{1a} and R^{1b} cycle may be further substituted with 1, 2, or 3—F atoms. In several embodiments, a C₁₋₃ alkyl substituent of the R^{1a} and R^{1b} cycle may be further substituted with a C₁₋₃ alkoxy. In several embodiments, a C₁₋₃ alkyl substituent of the R^{1a} and R^{1b} cycle may be further substituted with a C₁ alkoxy, C₂ alkoxy, or C₃ alkoxy (providing for example, C₁₋₃ alkylene-C₁ alkoxy, C₁₋₃ alkylene-C₂ alkoxy, C₁₋₃ alkylene-C₃ alkoxy, C₁ alkylene-C₁ alkoxy, C₂ alkylene-C₁ alkoxy, etc.). In several embodiments, a C₁₋₃ alkoxy substituent of the R^{1a} and R^{1b} cycle may be further substituted with a C₁₋₃ alkoxy. In several embodiments, a C₁₋₃ alkoxy substituent of the R^{1a} and R^{1b} cycle may be further substituted with a C₁ alkoxy, C₂ alkoxy, or C₃ alkoxy. As disclosed elsewhere herein, the R^{1a} and R^{1b}cycle may be cycloalkyl, cycloalkenyl, or heterocyclyl. In several embodiments, the R^{1a} and R^{1b} cycle is substituted with one or more substituents, each of which independently is:

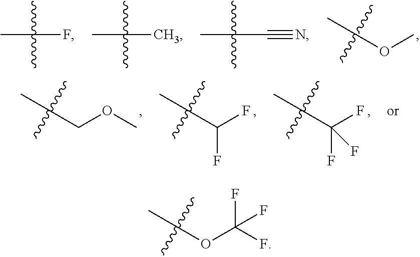

In several embodiments, the R^{1a} and R^{1b} cycle is substituted with one or more substituents, each of which independently is:

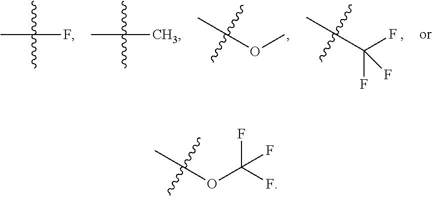

In several embodiments, the R^{1a} and R^{1b} cycle is substituted with one or more substituents, each of which independently is:

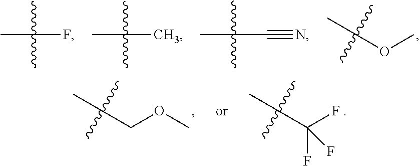

In several embodiments, the R^{1a} and R^{1b} cycle is substituted with one or more substituents, each of which independently is:

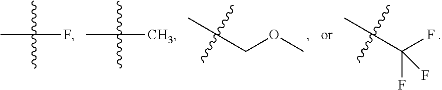

Provided herein as Embodiment 94 is the compound or salt of any one of Embodiments 1 to 33 and 71 to 93, wherein R^{1a} and R^{1b} together form a cycle that is substituted and each substituent of the cycle independently is halogen, —OH, oxo, —N(R^{b})₂, C₁₋₃ alkyl, or C₁₋₃alkoxy, wherein each of the C₁₋₃ alkyl and C₁₋₃ alkoxy may be unsubstituted or substituted with 1 to 6 halogen groups or C₁₋₃ alkoxy.

Provided herein as Embodiment 95 is the compound or salt of any one of Embodiments 1 to 33 and 71 to 94, wherein R^{1a} and R^{1b} together form a cycle that is substituted and each substituent of the cycle independently is C₁₋₃ alkyl or C₁₋₃alkoxy, wherein each of the C₁₋₃ alkyl and C₁₋₃ alkoxy may be unsubstituted or substituted with 1 to 3 halogen groups or C₁₋₃ alkoxy.

Provided herein as Embodiment 96 is the compound or salt of any one of Embodiments 1 to 33 and 71 to 95, wherein R^{1a} and R^{1b} together form a cycle that is substituted and each substituent of the cycle independently is —F, —OH, oxo, methyl, methoxy, or —OCF₃. For example, in several embodiments, each substituent of the R^{1a} and R^{1b} cycle independently is —OH, methyl, or —OMe. In several embodiments, each substituent of the R^{1a} and R^{1b} cycle independently is —F, methyl, or —OMe. In several embodiments, each substituent of the R^{1a} and R^{1b} cycle independently is —F, methyl, or —OCF₃. In several embodiments, each substituent of the R^{1a} and R^{1b} cycle independently is —F, —OH, or —OMe. In several embodiments, each substituent of the R^{1a} and R^{1b} cycle independently is —F, —OH, or methyl. In several embodiments, each substituent of the R^{1a} and R^{1b} cycle independently is —F or —OH. In several embodiments, each substituent of the R^{1a} and R^{1b} cycle independently is —F or methyl. In several embodiments, each substituent of the R^{1a} and R^{1b} cycle independently is —F or —OMe. In several embodiments, each substituent of the R^{1a} and R^{1b} cycle independently is —OH or methyl. In several embodiments, each substituent of the R^{1a} and R^{1b} cycle independently is —OH or —OMe. In several embodiments, each substituent of the R^{1a} and R^{1b} cycle independently is methyl or —OMe. In several embodiments, the R^{1a} and R^{1b} cycle is substituted with —F. In several embodiments, the R^{1a} and R^{1b} cycle is substituted with -OMe. In several embodiments, the R^{1a} and R^{1b} cycle is substituted with methyl (—Me). In several embodiments, the R^{1a} and R^{1b} cycle together with its substituents form a 4,4-difluorohexyl. In several embodiments, the R^{1a} and R^{1b} cycle together with its substituents form an N-methyl-piperidinyl. In several embodiments, the R^{1a} and R^{1b} cycle together with its substituents form an N-methyl-pyrrolidinyl. In several embodiments, the R^{1a} and R^{1b} cycle together with its substituents form an N-methyl-azetidinyl.

Provided herein as Embodiment 97 is the compound or salt of any one of Embodiments 1 to 33 and 71 to 91, wherein R^{1a} and R^{1b} together form a cycle that is unsubstituted.

Provided herein as Embodiment 98 is the compound or salt of any one of Embodiments 1 to 97, wherein R^{1c} is substituted with 1, 2, 3, or 4 substituents. In several embodiments, the substituents are each the same. In several embodiments, the substituents are each different. In several embodiments, one of substituents is different from the other substituents.

Provided herein as Embodiment 99 is the compound or salt of any one of Embodiments 1 to 97, wherein R^{1c} is substituted with 1, 2, or 3 substituents.

Provided herein as Embodiment 100 is the compound or salt of any one of Embodiments 1 to 97, wherein R^{1c} is substituted with 1 or 2 substituents.

Provided herein as Embodiment 101 is the compound or salt of any one of Embodiments 1 to 97, wherein R^{1c} is substituted with 1 substituent.

Provided herein as Embodiment 102 is the compound or salt of any one of Embodiments 1 to 97, wherein R^{1c} is unsubstituted.

Provided herein as Embodiment 103 is the compound or salt of any one of Embodiments 1 to 97, wherein R^{1c} is —H.

Provided herein as Embodiment 104 is the compound or salt of any one of Embodiments 1 to 102, wherein R^{1c} is C₁₋₃ alkyl.

Provided herein as Embodiment 105 is the compound or salt of any one of Embodiments 1 to 102, wherein R^{1c} is —CF₃.

Provided herein as Embodiment 106 is the compound or salt of any one of Embodiments 1 to 97, wherein R^{1c} is —CH₃.

Provided herein as Embodiment 107 is the compound or salt of any one of Embodiments 1 to 106, wherein R² is unsubstituted or substituted C₃₋₆cycloalkyl, unsubstituted or substituted C₆₋₁₀aryl, unsubstituted or substituted heteroaryl, or unsubstituted or substituted heterocyclyl. For example, in several embodiments, R² is C₃₋₆cycloalkyl, heteroaryl having 5 to 10 ring members, or heterocyclyl having 5 to 10 ring members. In several embodiments, R² is heteroaryl having 5 to 10 ring members or heterocyclyl having 5 to 10 ring members, in several embodiments, R² is heteroaryl having 5 to 10 ring members. In several embodiments, R² is C₃₋₆cycloalkyl, heteroaryl having 5 to 10 ring members, or heterocyclyl having 5 to 10 ring members. In several embodiments, R² is C₆₋₁₀aryl or heteroaryl having 5 to 10 ring members. In several embodiments, R² is C₃₋₆cycloalkyl or heteroaryl having 5 to 10 ring members. In several embodiments, R² is C₃₋₆cycloalkyl, C₆₋₁₀aryl, or heteroaryl having 5 to 10 ring members. In several embodiments, R² is pyrazolopyridinyl (e.g., pyrazolo[3,4-b]pyridinyl, 1H-pyrazolo[3,4-b]pyridin-5-yl, etc.). In several embodiments, R² is pyrazolyl. In several embodiments, R² is indazolyl. In several embodiments, R² is triazolopyridinyl (e.g., 3H-[1,2,3]triazolo[4,5-b]pyridin-6-yl, etc.). In several embodiments, R² is imidazopyridinyl (e.g., imidazo[1,2-a]pyridin-6-yl, etc.). In several embodiments, R² is benzoxazolyl. In several embodiments, R² is imidazo[1,2-a]pyridinyl. In several embodiments, R² is pyrimidinonyl (e.g., pyrimidin-4(3H)-one). In several embodiments, R² is isoquinolin-1(2H)-one. In several embodiments, R² is quinoxalinyl. In several embodiments, R² is pyrimidinyl.

Provided herein as Embodiment 108 is the compound or salt of any one of Embodiments 1 to 107, wherein R is unsubstituted or substituted heteroaryl or unsubstituted or substituted heterocyclyl and the R²heteroaryl or heterocyclyl includes 1, 2, 3, 4, or 5 heteroatom ring members. For example, in several embodiments, includes 5 heteroatom ring members. In several embodiments, includes 4 heteroatom ring members. In several embodiments, includes 2 heteroatom ring members. In several embodiments, includes 2 heteroatom ring members. In several embodiments, includes 1 heteroatom ring member. In several embodiments, R² comprises 1 to 5 heteroatom ring members. In several embodiments, R² comprises 1 to 4 heteroatom ring members. In several embodiments, R² comprises 1 to 3 heteroatom ring members. In several embodiments, R² comprises 1 or 2 heteroatom ring members. In several embodiments, R² comprises 2 to 5 heteroatom ring members. In several embodiments, R² comprises 3 to 5 heteroatom ring members. In several embodiments, R² comprises 3 to 4 heteroatom ring members. In several embodiments, R² comprises nitrogen atoms as the only ring heteroatoms. In several embodiments, R² comprises one nitrogen atom as a ring heteroatom. In several embodiments, R² comprises two nitrogen atoms as a ring heteroatoms. In several embodiments, R² comprises three nitrogen atoms as a ring heteroatoms. In several embodiments, R² comprises four nitrogen atoms as a ring heteroatoms. In several embodiments, R² comprises five nitrogen atoms as a ring heteroatoms. In several embodiments, R² comprises one oxygen ring heteroatom and the remaining ring heteroatoms are nitrogen atoms. In several embodiments, R² comprises oxygen atoms as the only ring heteroatoms.

Provided herein as Embodiment 109 is the compound or salt of any one of Embodiments 1 to 107, wherein R is unsubstituted or substituted heteroaryl or unsubstituted or substituted heterocyclyl and the R²heteroaryl or heterocyclyl includes 1, 2, 3, or 4 heteroatom ring members.

Provided herein as Embodiment 110 is the compound or salt of any one of Embodiments 1 to 107, wherein R is unsubstituted or substituted heteroaryl or unsubstituted or substituted heterocyclyl and the R²heteroaryl or heterocyclyl includes 1, 2, or 3 heteroatom ring members.

Provided herein as Embodiment 111 is the compound or salt of any one of Embodiments 1 to 107, wherein R is unsubstituted or substituted heteroaryl or unsubstituted or substituted heterocyclyl and the R₂ heteroaryl or heterocyclyl includes 1 or 2 heteroatom ring members.

Provided herein as Embodiment 112 is the compound or salt of any one of Embodiments 1 to 107, wherein R is unsubstituted or substituted heteroaryl or unsubstituted or substituted heterocyclyl and the R₂ heteroaryl or heterocyclyl includes 1 heteroatom ring member.

Provided herein as Embodiment 113 is the compound or salt of any one of Embodiments 1 to 112, wherein R is unsubstituted or substituted heteroaryl or unsubstituted or substituted heterocyclyl and each ring member heteroatom of the R² ring independently is nitrogen, oxygen, or —S(O)_{y}—, where y is 0, 1, or 2.

Provided herein as Embodiment 114 is the compound or salt of any one of Embodiments 1 to 113, wherein R is unsubstituted or substituted heteroaryl or unsubstituted or substituted heterocyclyl and each heteroatom of the R² ring independently is nitrogen or oxygen.

Provided herein as Embodiment 115 is the compound or salt of any one of Embodiments 1 to 113, wherein R is unsubstituted or substituted heteroaryl or unsubstituted or substituted heterocyclyl and each heteroatom of the R² ring is nitrogen.

Provided herein as Embodiment 116 is the compound or salt of any one of Embodiments 1 to 107 and 113 to 115, wherein R² is unsubstituted or substituted heteroaryl or unsubstituted or substituted heterocyclyl comprising four nitrogen ring atoms.

Provided herein as Embodiment 117 is the compound or salt of any one of Embodiments 1 to 110 and 113 to 115, wherein R² is unsubstituted or substituted heteroaryl or unsubstituted or substituted heterocyclyl comprising three nitrogen ring atoms.

Provided herein as Embodiment 118 is the compound or salt of any one of Embodiments 1 to 111 and 113 to 115, wherein R² is unsubstituted or substituted heteroaryl or unsubstituted or substituted heterocyclyl comprising two nitrogen ring atoms.

Provided herein as Embodiment 119 is the compound or salt of any one of Embodiments 1 to 115, wherein R is unsubstituted or substituted heteroaryl or unsubstituted or substituted heterocyclyl comprising one nitrogen ring atom.

Provided herein as Embodiment 120 is the compound or salt of any one of Embodiments 1 to 114, wherein R² is unsubstituted or substituted heteroaryl or unsubstituted or substituted heterocyclyl and each heteroatom of the R² ring is oxygen.

Provided herein as Embodiment 121 is the compound or salt of any one of Embodiments 1 to 120, wherein R² is unsubstituted or substituted fused bicyclic heteroaryl or unsubstituted or substituted fused bicyclic heterocyclyl. For example, in several embodiments, R² is fused bicyclic heteroaryl. In several embodiments, the fused bicyclic R² comprises a 6-membered ring. In several embodiments, the fused bicyclic R² comprises a 5-membered ring. In several embodiments, R² comprises a 6-membered ring and a 5-membered ring, which are fused. In several embodiments, the fused bicyclic R² is a 5,6-member fused ring. In several embodiments, the fused bicyclic R² is a 6,6-member fused ring.

Provided herein as Embodiment 122 is the compound or salt of any one of Embodiments 1 to 120, wherein R is unsubstituted or substituted heteroaryl having 5 to 9 ring-members or unsubstituted or substituted heterocyclyl having 5 to 9 ring-members. In several embodiments, the fused bicyclic R² is a 5,6-member fused ring.

Provided herein as Embodiment 123 is the compound or salt of any one of Embodiments 1 to 120, wherein R² is unsubstituted or substituted heteroaryl having 5 to 10 ring-members or unsubstituted or substituted heterocyclyl having 5 to 10 ring-members. In several embodiments, the fused bicyclic R² is a 6,6-member fused ring.

Provided herein as Embodiment 124 is the compound or salt of any one of Embodiments 1 to 121, wherein R² is unsubstituted or substituted heteroaryl having 8 to 10 ring-members or unsubstituted or substituted heterocyclyl having 8 to 10 ring-members.

Provided herein as Embodiment 125 is the compound or salt of any one of Embodiments 1 to 121, wherein R² is unsubstituted or substituted heteroaryl having 9 to 10 ring-members or unsubstituted or substituted heterocyclyl having 9 to 10 ring-members.

Provided herein as Embodiment 126 is the compound or salt of any one of Embodiments 1 to 121, wherein R is unsubstituted or substituted heteroaryl having 9 ring-members or unsubstituted or substituted heterocyclyl having 9 ring-members.

Provided herein as Embodiment 127 is the compound or salt of any one of Embodiments 1 to 121, wherein R² is unsubstituted or substituted heteroaryl having 10 ring-members or unsubstituted or substituted heterocyclyl having 10 ring-members.

Provided herein as Embodiment 128 is the compound or salt of any one of Embodiments 1 to 120, wherein R² is a 6-ring member heteroaryl or a is a 6-ring member heterocyclyl.

Provided herein as Embodiment 129 is the compound or salt of any one of Embodiments 1 to 107 and 109 to 120, wherein R² is unsubstituted or substituted heteroaryl having 5 ring members or unsubstituted or substituted heterocyclyl having 5 ring members.

Provided herein as Embodiment 130 is the compound or salt of any one of Embodiments 1 to 129, wherein R² is unsubstituted or substituted heteroaryl.

Provided herein as Embodiment 131 is the compound or salt of any one of Embodiments 1 to 129, wherein R² is unsubstituted or substituted heterocyclyl.

Provided herein as Embodiment 132 is the compound or salt of any one of Embodiments 1 to 106, wherein R² is unsubstituted or substituted C₃₋₆cycloalkyl. For example, in several embodiments, R² is a C₄₋₆ cycloalkyl. In several embodiments, R² is a C₃₋₅ cycloalkyl. In several embodiments, R² is a C₄₋₆ cycloalkyl. In several embodiments, R² is a C₃₋₄ cycloalkyl. In several embodiments, R² is a C₅₋₆ cycloalkyl. In several embodiments, R² is a C₃ cycloalkyl (e.g., cyclopropyl). In several embodiments, R² is a C₄ cycloalkyl (e.g., cyclobutyl). In several embodiments, R² is a C₅ cycloalkyl. In several embodiments, R² is a C₆ cycloalkyl.

Provided herein as Embodiment 133 is the compound or salt of any one of Embodiments 1 to 106, wherein R² is unsubstituted or substituted C₃₋₁₀cycloalkenyl. For example, in several embodiments, R² is a C₄₋₆ cycloalkenyl. In several embodiments, R² is a C₃₋₅ cycloalkenyl. In several embodiments, R² is a C₄₋₆ cycloalkenyl. In several embodiments, R² is a C₃₋₄ cycloalkenyl. In several embodiments, R² is a C₅₋₆ cycloalkenyl. In several embodiments, R² is a C₅₋₁₀cycloalkenyl. In several embodiments, R² is a C₃ cycloalkenyl. In several embodiments, R² is a C₄ cycloalkenyl. In several embodiments, R² is a Cs cycloalkenyl. In several embodiments, R² is a C₆ cycloalkenyl. In several embodiments, R² is a C₇ cycloalkenyl. In several embodiments, R² is a C₅ cycloalkenyl. In several embodiments, R² is a C₉ cycloalkenyl. In several embodiments, R² is a C₁₀ cycloalkenyl.

Provided herein as Embodiment 134 is the compound or salt of any one of Embodiments 1 to 106, wherein R² is unsubstituted or substituted C₆₋₁₀aryl. For example, in several embodiments, R² is a C₆ aryl (e.g., phenyl). In several embodiments, R² is a C₁₀ aryl.

Provided herein as Embodiment 135 is the compound or salt of any one of Embodiments 1 to 134, wherein R² is substituted with 1, 2, 3, 4, or 5 substituents. For example, in several embodiments, R² is substituted with 1 to 5 substituents. In several embodiments, R² is substituted with 1 to 4 substituents. In several embodiments, R² is substituted with 1 to 3 substituents. In several embodiments, R² is substituted with 1 to 2 substituents. In several embodiments, R² is substituted with 2 to 4 substituents. In several embodiments, R² is substituted with 2 to 5 substituents. In several embodiments, R² is substituted with 3 to 5 substituents. In several embodiments, R² is substituted with 4 to 5 substituents. In several embodiments, R² is substituted with 3 to 4 substituents. In several embodiments, R² is substituted with 2 to 3 substituents.

Provided herein as Embodiment 136 is the compound or salt of any one of Embodiments 1 to 134, wherein R² is substituted with 1, 2, 3, or 4 substituents.

Provided herein as Embodiment 137 is the compound or salt of any one of Embodiments 1 to 134, wherein R² is substituted with 1, 2, or 3 substituents.

Provided herein as Embodiment 138 is the compound or salt of any one of Embodiments 1 to 134, wherein R² is substituted with 1 or 2 substituents.

Provided herein as Embodiment 139 is the compound or salt of any one of Embodiments 1 to 134, wherein R² is substituted with 1 substituent.

Provided herein as Embodiment 140 is the compound or salt of any one of Embodiments 1 to 139, wherein, when R² is substituted, each R² substituent independently is halogen, —OH, oxo, C₁₋₃alkyl, or C₁₋₃alkoxy. For example, in several embodiments, each R² substituent independently is halogen, —OH, oxo, or C₁₋₃alkyl. In several embodiments, each R² substituent independently is halogen, —OH, oxo, C₁₋₃ alkyl, or C₁₋₃alkoxy. In several embodiments, each R² substituent independently is halogen, —OH, oxo, or C₁₋₃alkoxy. In several embodiments, each R² substituent independently is halogen, —OH, C₁₋₃alkyl, or C₁₋₃ alkoxy. In several embodiments, each R² substituent independently is halogen, oxo, C₁₋₃alkyl, or C₁₋₃ alkoxy. In several embodiments, each R² substituent independently is —OH, oxo, C₁₋₃alkyl, or C₁₋₃alkoxy. In several embodiments, each R² substituent independently is oxo, C₁₋₃alkyl, or C₁₋₃alkoxy. In several embodiments, each R² substituent independently is halogen, C₁₋₃alkyl, or C₁₋₃alkoxy. In several embodiments, each R² substituent independently is halogen, —OH, or C₁₋₃alkoxy. In several embodiments, each R² substituent independently is halogen, —OH, or oxo. In several embodiments, each R² substituent independently is halogen, —OH, oxo, C₁₋₃alkyl, or C₁₋₃alkoxy. In several embodiments, each R² substituent independently is halogen, oxo, or C₁₋₃alkyl. In several embodiments, each R² substituent independently is halogen or C₁₋₃alkyl. In several embodiments, each R² substituent independently is oxo or C₁₋₃alkyl. In several embodiments, each R² substituent independently is halogen or oxo. In several embodiments, each R² substituent independently is halogen. In several embodiments, a halogen substituent of R (or one or more substituents independently) is —F, —Cl, or —Br. In several embodiments, a halogen substituent of R² (or one or more substituents independently) is —F or —Cl. In several embodiments, R² is substituted with —F. In several embodiments, R² is substituted with C₁₋₃alkyl. In several embodiments, R² is substituted with C₁₋₂ alkyl. In several embodiments, R² is substituted with C₂₋₃ alkyl. In several embodiments, R² is substituted with methyl. In several embodiments, R² is substituted with C₂ alkyl (e.g., ethyl). In several embodiments, R² is substituted with C₃ alkyl. In several embodiments, R² is substituted with C₁₋₂ alkoxy. In several embodiments, R² is substituted with C₂₋₃ alkoxy. In several embodiments, R² is substituted with methoxy. In several embodiments, R² is substituted with C₂ alkoxy. In several embodiments, R² is substituted with C₃ alkoxy. In several embodiments, R² together with its substituents form N-methyl-pyrazolyl. In several embodiments, R² together with its substituents form an alkyl-pyrazolo[3,4-b]pyridine (e.g., 2-methyl-2H-pyrazolo[3,4-b]pyridinyl, 2-ethyl-2H-pyrazolo[3,4-b]61yridine-5-yl, 2-(2-propanyl)-2H-pyrazolo[3,4-b]61yridine-5-yl, 1-(2-propanyl)-1H-pyrazolo[3,4-b]61yridine-5-yl, etc.). In several embodiments, R² together with its substituents form a methyl-pyrazolo[3,4-b]pyridine. In several embodiments, R² together with its substituents form an alkyl-indazolyl (e.g., 1-methyl-1H-indazol-5-yl, 3-methyl-1H-indazol-5-yl, 6-methyl-1H-indazol-5-yl, 2-isopropyl-2H-indazol-5-yl, etc.). In several embodiments, R² together with its substituents form a methyl-indazolyl. In several embodiments, R² together with its substituents form a methyl-triazolopyridinyl (e.g., 2-methyl-2H-triazolo[4,5-b]pyridine-6-yl, 3-methyl-3H-[1,2,3]triazolo[4,5-b]pyridine-6-yl, etc.). In several embodiments, R² together with its substituents form a methyl-benzoxazolyl (e.g., 1,2-benzoxazol-5-yl, 2-methyl-1,3-benzoxazol-5-yl, etc.). In several embodiments, together with its substituents form a methyl-imidazo[1,2-a]pyridinyl (e.g. 2-methylimidazo[1,2-a]pyridine-6-yl, etc.). In several embodiments, R² together with its substituents form a methyl-pyrimidinonyl (e.g., 3-methylpyrimidin-4(3H)-one). In several embodiments, R² together with its substituents form a methyl-isoquinolin-onyl (e.g., 2-methylisoquinolin-1(2H)-one).

Provided herein as Embodiment 141 is the compound or salt of any one of Embodiments 1 to 139, wherein, when R² is substituted, each R² substituent independently is methyl, ethyl, or isopropyl.

Provided herein as Embodiment 142 is the compound or salt of any one of Embodiments 1 to 139, wherein, when R² is substituted, R² is substituted with methyl.

Provided herein as Embodiment 143 is the compound or salt of any one of Embodiments 1 to 139, wherein, when R² is substituted, R² is substituted with ethyl.

Provided herein as Embodiment 144 is the compound or salt of any one of Embodiments 1 to 139, wherein, when R² is substituted, R² is substituted with isopropyl.

Provided herein as Embodiment 145 is the compound or salt of any one of Embodiments 1 to 134, wherein R² is unsubstituted.

Provided herein as Embodiment 146 is the compound or salt of any one of Embodiments 1 to 106, wherein R² is

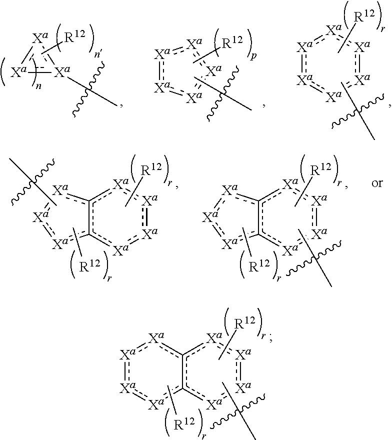

where
[symbol] indicates a double bond or single bond may be present;
each instance of X independently is nitrogen, oxygen, or carbon;
each instance of n, where present, is an integer selected from 1, 2, 3, 4, or 5;
each instance of n′, where present, is an integer selected from 1, 2, 3, 4, or 5;
each instance of p, where present, is an integer selected from 0, 1, 2, 3, or 4;
each instance of r, where present, is an integer selected from 0, 1, 2, 3, 4, or 5, wherein where two instances of r occur on the same R² group, the total of both r values combined does not exceed 5;
each instance of R¹², when present and attached to an N ring member, is C₁₋₆alkyl, C₁₋₆haloalkyl, C₁₋₆alkylene-OH, or C₁₋₆alkylene-O—C₁₋₆alkyl; and
each instance of R¹², when present and attached to a C ring member, independently is halogen, —OH, oxo, —N(R^{b})₂, C₁₋₆ alkyl, C₁₋₆ haloalkyl, C₁₋₆alkoxy, C₁₋₆alkylene-OH, or C₁₋₆alkylene-O—C₁₋₆alkyl. In several embodiments, a —N=ring nitrogen can be bonded to an R¹² to provide a cation. In several embodiments, no —N=ring nitrogen is bonded to an R¹² to provide a cation. In several embodiments, each instance of R¹², when present and attached to an N ring member, is C₁₋₆alkyl, C₁₋₆haloalkyl, C₁₋₆ alkylene-OH, or C₁₋₆alkylene-O—C₁₋₆alkyl. In several embodiments, each instance of R¹², when present and attached to an N ring member, is C₁₋₃ alkyl, C₁₋₃ haloalkyl, C₁₋₃ alkylene-OH, or C₁₋₃ alkylene-O—C₁₋₃ alkyl. In several embodiments, each instance of R¹², when present and attached to an N ring member, is C₁₋₃ alkyl, C₁₋₃ haloalkyl, or C₁₋₃ alkylene-OH. In several embodiments, each instance of R¹², when present and attached to an N ring member, is C₁₋₃ alkyl, C₁₋₃ haloalkyl, or C₁₋₃ alkylene-O—C₁₋₃ alkyl. In several embodiments, each instance of R¹², when present and attached to an N ring member, is C₁₋₃ alkyl, C₁₋₃ alkylene-OH, or C₁₋₃ alkylene-O—C₁₋₃ alkyl. In several embodiments, each instance of R¹², when present and attached to an N ring member, is C₁₋₃ haloalkyl, C₁₋₃ alkylene-OH, or C₁₋₃ alkylene-O—C₁₋₃ alkyl. In several embodiments, each instance of R¹², when present and attached to an N ring member, is C₁₋₃ alkyl or C₁₋₃ haloalkyl. In several embodiments, an instance of R¹², when present and attached to an N ring member, is C₁₋₃ alkyl (e.g., methyl, ethyl, propyl, or isopropyl). In several embodiments, each instance of R¹², when present and attached to a C ring member, independently is halogen, —OH, oxo, —N(R^{b})₂, C₁₋₆ alkyl, C₁₋₆ haloalkyl, C₁₋₆ alkoxy, or C₁₋₆alkylene-OH. In several embodiments, each instance of R¹², when present and attached to a C ring member, independently is halogen, —OH, oxo, —N(R^{b})₂, C₁₋₆ alkyl, C₁₋₆ haloalkyl, C₁₋₆ alkoxy, or C₁₋₆alkylene-O—C₁₋₆alkyl. In several embodiments, each instance of R¹², when present and attached to a C ring member, independently is halogen, —OH, oxo, —N(R^{b})₂, C₁₋₆ alkyl, C₁₋₆ haloalkyl, C₁₋₆alkylene-OH, or C₁₋₆alkylene-O—C₁₋₆alkyl. In several embodiments, each instance of R¹², when present and attached to a C ring member, independently is halogen, —OH, oxo, —N(R^{b})₂, C₁₋₆ alkyl, C₁₋₆ alkoxy, C₁₋₆alkylene-OH, or C₁₋₆alkylene-O—C₁₋₆alkyl. In several embodiments, each instance of R¹², when present and attached to a C ring member, independently is halogen, —OH, oxo, —N(R^{b})₂, C₁₋₆ haloalkyl, C₁₋₆ alkoxy, C₁₋₆alkylene-OH, or C₁₋₆alkylene-O—C₁₋₆alkyl. In several embodiments, each instance of R¹², when present and attached to a C ring member, independently is halogen, —OH, oxo, C₁₋₆ alkyl, C₁₋₆ haloalkyl, C₁₋₆ alkoxy, C₁₋₆alkylene-OH, or C₁₋₆ alkylene-O—C₁₋₆alkyl. In several embodiments, each instance of R¹², when present and attached to a C ring member, independently is halogen, —OH, —N(R^{b})₂, C₁₋₆ alkyl, C₁₋₆ haloalkyl, C₁₋₆ alkoxy, C₁₋₆alkylene-OH, or C₁₋₆alkylene-O—C₁₋₆alkyl. In several embodiments, each instance of R¹², when present and attached to a C ring member, independently is halogen, oxo, —N(R^{b})₂, C₁₋₆ alkyl, C₁₋₆ haloalkyl, C₁₋₆ alkoxy, C₁₋₆alkylene-OH, or C₁₋₆alkylene-O—C₁₋₆alkyl. In several embodiments, each instance of R¹², when present and attached to a C ring member, independently is halogen, —OH, oxo, —N(R^{b})₂, C₁₋₆ alkyl, C₁₋₆ haloalkyl, or C₁₋₆ alkoxy. In several embodiments, each instance of R¹², when present and attached to an C ring member, is C₁₋₆alkyl, C₁₋₆haloalkyl, C₁₋₆alkylene-OH, or C₁₋₆alkylene-O—C₁₋₆alkyl. In several embodiments, each instance of R¹², when present and attached to an C ring member, is C₁₋₃ alkyl, C₁₋₃ haloalkyl, C₁₋₃ alkylene-OH, or C₁₋₃ alkylene-O—C₁₋₃ alkyl. In several embodiments, each instance of R¹², when present and attached to an C ring member, is C₁₋₃ alkyl, C₁₋₃ haloalkyl, or C₁₋₃ alkylene-OH. In several embodiments, each instance of R¹², when present and attached to an C ring member, is C₁₋₃ alkyl, C₁₋₃ haloalkyl, or C₁₋₃ alkylene-O—C₁₋₃ alkyl. In several embodiments, each instance of R¹², when present and attached to an C ring member, is C₁₋₃ alkyl, C₁₋₃ alkylene-OH, or C₁₋₃ alkylene-O—C₁₋₃ alkyl. In several embodiments, each instance of R¹², when present and attached to an C ring member, is C₁₋₃ haloalkyl, C₁₋₃ alkylene-OH, or C₁₋₃ alkylene-O-C₁₋₃ alkyl. In several embodiments, each instance of R¹², when present and attached to an C ring member, is C₁₋₃ alkyl or C₁₋₃ haloalkyl. In several embodiments, an instance of R¹², when present and attached to an C ring member, is C₁₋₃ alkyl. In several embodiments, an instance of R¹², when present and attached to an C ring member, is C₁₋₂ alkyl. In several embodiments, an instance of R¹², when present and attached to an C ring member, is C₂₋₃ alkyl. In several embodiments, an instance of R¹², when present and attached to an C ring member, is methyl. In several embodiments, an instance of R¹², when present and attached to an C ring member, is C₂ alkyl. In several embodiments, an instance of R¹², when present and attached to an C ring member, is C₃ alkyl. In several embodiments, an instance of R¹², when present and attached to an C ring member, is C₁₋₂haloalkyl. In several embodiments, an instance of R¹², when present and attached to an C ring member, is C₁ haloalkyl. In several embodiments, an instance of R¹², when present and attached to an C ring member, is C₂haloalkyl. In several embodiments, an instance of R¹², when present and attached to an C ring member, is C₃ haloalkyl. In several embodiments, the R¹² halogen is —F, —Cl, or —Br. In several embodiments, the R¹² halogen is —F or —Cl. In several embodiments, the R¹² halogen is —F.

Provided herein as Embodiment 147 is the compound or salt of any one of Embodiments 1 to 17, 19 to 106, and 146, wherein Formula (A) is further represented by the compound of Formula (A2i):

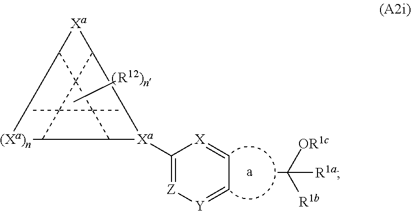

where
“ [symbol] ” indicates a double bond or single bond may be present;
each instance of X^{a} independently is nitrogen, oxygen, or carbon;
each instance of n, where present, is an integer selected from 1, 2, 3, 4, or 5; and
each instance of n′, where present, is an integer selected from 1, 2, 3, 4, or 5;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, oxo, C₁₋₃alkyl, C₁₋₃haloalkyl, C₁₋₃alkylene-OH, or C₁₋₃alkylene-O—C₁₋₃alkyl. In several embodiments, a —N=ring nitrogen can be bonded to an R¹² to provide a cation. In several embodiments, no —N=ring nitrogen is bonded to an R¹² to provide a cation. In several embodiments, the variables of the compound of Formula (A2i) are as defined in any one of Embodiments 1 to 17, 19 to 106, and 146.

Provided herein as Embodiment 148 is the compound or salt of any one of Embodiments 1 to 17, 19 to 106, and 146, wherein Formula (A) is further represented by the compound of Formula (A2ii):

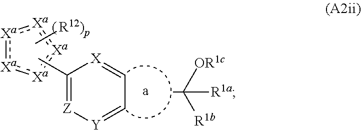

where
“ [symbol] ” indicates a double bond or single bond may be present;
each instance of X^{a} independently is nitrogen, oxygen, or carbon;
each instance of p is an integer selected from 0, 1, 2, 3, or 4;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, oxo, C₁₋₃alkyl, C₁₋₃haloalkyl, C₁₋₃alkylene-OH, or C₁₋₃alkylene-O—C₁₋₃alkyl. In several embodiments, a —N=ring nitrogen can be bonded to an R¹² to provide a cation. In several embodiments, no —N=ring nitrogen is bonded to an R¹² to provide a cation. In several embodiments, the variables of the compound of Formula (A2ii) are as defined in any one of Embodiments 1 to 17, 19 to 106, and 146.

Provided herein as Embodiment 149 is the compound or salt of any one of Embodiments 1 to 17, 19 to 106, and 146, wherein Formula (A) is further represented by the compound of Formula (A2iii):

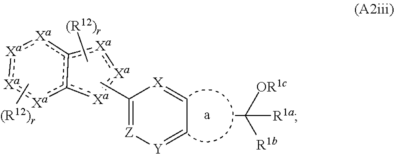

where
“ [symbol] ” indicates a double bond or single bond may be present;
each instance of X^{a} independently is nitrogen, oxygen, or carbon;
each instance of r is an integer selected from 0, 1, 2, 3, 4, or 5, wherein, where two instances of r occur on the Formula (A2iii) compound, the total of both r values combined does not exceed 5;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, oxo, C₁₋₃alkyl, C₁₋₃haloalkyl, C₁₋₃alkylene-OH, or C₁₋₃alkylene-O—C₁₋₃alkyl. In several embodiments, a —N=ring nitrogen can be bonded to an R¹² to provide a cation. In several embodiments, no —N=ring nitrogen is bonded to an R¹² to provide a cation. In several embodiments, the variables of the compound of Formula (A2iii) are as defined in any one of Embodiments 1 to 17, 19 to 106, and 146.

Provided herein as Embodiment 150 is the compound or salt of any one of Embodiments 1 to 17, 19 to 106, and 146, wherein Formula (A) is further represented by the compound of Formula (A2iv):

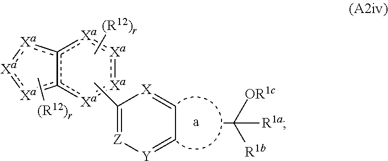

where
“ [symbol] ” indicates a double bond or single bond may be present;
each instance of X^{a} independently is nitrogen, oxygen, or carbon;
each instance of r is an integer selected from 0, 1, 2, 3, 4, or 5, wherein, where two instances of r occur on the Formula (A2iv) compound, the total of both r values combined does not exceed 5;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, oxo, C₁₋₃alkyl, C₁₋₃haloalkyl, C₁₋₃alkylene-OH, or C₁₋₃alkylene-O—C₁₋₃alkyl. In several embodiments, a —N=ring nitrogen can be bonded to an R¹² to provide a cation. In several embodiments, no —N=ring nitrogen is bonded to an R¹² to provide a cation. In several embodiments, the variables of the compound of Formula (A2iv) are as defined in any one of Embodiments 1 to 17, 19 to 106, and 146.

Provided herein as Embodiment 151 is the compound or salt of any one of Embodiments 1 to 17, 19 to 106, and 146, wherein Formula (A) is further represented by the compound of Formula (A2v):

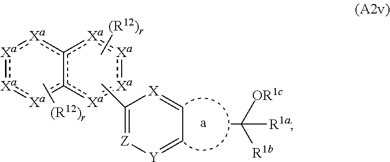

where
“ [symbol] ” indicates a double bond or single bond may be present;
each instance of X^{a} independently is nitrogen, oxygen, or carbon;
each instance of r is an integer selected from 0, 1, 2, 3, 4, or 5, wherein, where two instances of r occur on the Formula (A2v) compound, the total of both r values combined does not exceed 5;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, oxo, C₁₋₃alkyl, C₁₋₃haloalkyl, C₁₋₃alkylene-OH, or C₁₋₃alkylene-O—C₁₋₃alkyl. In several embodiments, a —N=ring nitrogen can be bonded to an R¹² to provide a cation. In several embodiments, no —N=ring nitrogen is bonded to an R¹² to provide a cation. In several embodiments, the variables of the compound of Formula (A2v) are as defined in any one of Embodiments 1 to 17, 19 to 106, and 146.

Provided herein as Embodiment 152 is the compound or salt of any one of Embodiments 1 to 106 and 146, wherein R² is

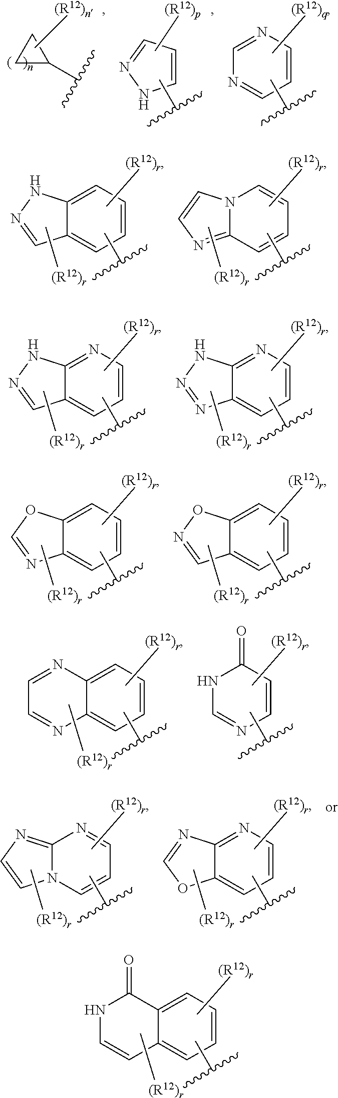

where
each instance of n, where present, is an integer selected from 1, 2, 3, 4, or 5;
each instance of n′, where present, is an integer selected from 0, 1, 2, 3, 4, or 5;
each instance of p, where present, is an integer selected from 0, 1, 2, 3, or 4;
each instance of q, where present, is an integer selected from 0, 1, 2, 3, 4, or 5;
each instance of r, where present, is an integer selected from 0, 1, 2, 3, 4, or 5, wherein where two instances of r occur on the same R² group, the total of both r values combined does not exceed 5;
each instance of s, where present, is an integer selected from 0, 1, or 2;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl;
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl;
wherein an —H on any ring member can be replaced with R¹². In several embodiments, a —N=ring nitrogen can be bonded to an R¹² to provide a cation. In several embodiments, no —N=ring nitrogen is bonded to an R¹² to provide a cation. In several embodiments, the variables for each R² structure provided are as defined in any one of Embodiments 1 to 106 and 146.

Provided herein as Embodiment 153 is the compound or salt of any one of Embodiments 1 to 106 and 146, wherein R² is:

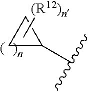

where
n is an integer selected from 1, 2, 3, 4, or 5;
n′ is an integer selected from 0, 1, 2, 3, 4, or 5;
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl.

Provided herein as Embodiment 154 is the compound or salt of any one of Embodiments 1 to 106 and 146, wherein R² is:

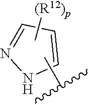

where
p is an integer selected from 0, 1, 2, 3, or 4;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl.

Provided herein as Embodiment 155 is the compound or salt of any one of Embodiments 1 to 106 and 146, wherein R² is:

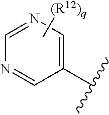

where
q is an integer selected from 0, 1, 2, 3, 4, or 5;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl. In several embodiments, a —N=ring nitrogen can be bonded to an R¹² to provide a cation. In several embodiments, no —N=ring nitrogen is bonded to an R¹² to provide a cation.

Provided herein as Embodiment 156 is the compound or salt of any one of Embodiments 1 to 106 and 146, wherein R² is:

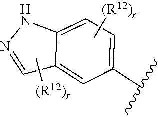

where
each instance of r is an integer selected from 0, 1, 2, or 3;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl. In several embodiments, a —N=ring nitrogen can be bonded to an R¹² to provide a cation. In several embodiments, no —N=ring nitrogen is bonded to an R¹² to provide a cation.

Provided herein as Embodiment 157 is the compound or salt of any one of Embodiments 1 to 106 and 146, wherein R² is:

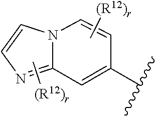

where
each instance of r is an integer selected from 0, 1, 2, or 3;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O-C₁₋₃alkyl. In several embodiments, a —N=ring nitrogen can be bonded to an R¹² to provide a cation. In several embodiments, no —N=ring nitrogen is bonded to an R¹² to provide a cation.

Provided herein as Embodiment 158 is the compound or salt of any one of Embodiments 1 to 106 and 146, wherein R² is:

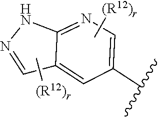

where
each instance of r is an integer selected from 0, 1, 2, or 3;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl. In several embodiments, a —N=ring nitrogen can be bonded to an R¹² to provide a cation. In several embodiments, no —N=ring nitrogen is bonded to an R¹² to provide a cation.

Provided herein as Embodiment 159 is the compound or salt of any one of Embodiments 1 to 106 and 146, wherein R² is

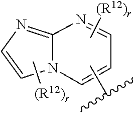

where
each instance of r is an integer selected from 0, 1, 2, or 3;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl. In several embodiments, a —N=ring nitrogen can be bonded to an R¹² to provide a cation. In several embodiments, no —N=ring nitrogen is bonded to an R¹² to provide a cation.

Provided herein as Embodiment 160 is the compound or salt of any one of Embodiments 1 to 106 and 146, wherein R² is:

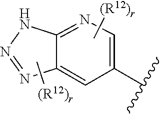

where
each instance of r is an integer selected from 0, 1, 2, or 3;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl. In several embodiments, a —N=ring nitrogen can be bonded to an R¹² to provide a cation. In several embodiments, no —N=ring nitrogen is bonded to an R¹² to provide a cation.

Provided herein as Embodiment 161 is the compound or salt of any one of Embodiments 1 to 106 and 146, wherein R² is:

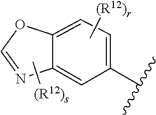

where
each instance of r, where present, is an integer selected from 0, 1, 2, or 3;
each instance of s, where present, is an integer selected from 0, 1, or 2;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl. In several embodiments, a —N=ring nitrogen can be bonded to an R¹² to provide a cation. In several embodiments, no —N=ring nitrogen is bonded to an R¹² to provide a cation.

Provided herein as Embodiment 162 is the compound or salt of any one of Embodiments 1 to 106 and 146, wherein R² is:

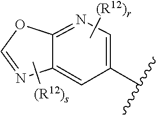

where
each instance of r, where present, is an integer selected from 0, 1, or 2;
each instance of s, where present, is an integer selected from 0 or 1;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl. In several embodiments, a —N=ring nitrogen can be bonded to an R¹² to provide a cation. In several embodiments, no —N=ring nitrogen is bonded to an R¹² to provide a cation.

Provided herein as Embodiment 163 is the compound or salt of any one of Embodiments 1 to 106 and 146, wherein R² is:

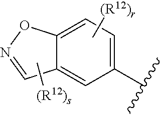

where
each instance of r, where present, is an integer selected from 0, 1, 2, or 3;
each instance of s, where present, is an integer selected from 0, 1, or 2;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl.

Provided herein as Embodiment 164 is the compound or salt of any one of Embodiments 1 to 106 and 146, wherein R² is:

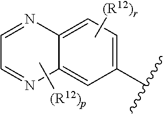

where
each instance of p, where present, is an integer selected from 0, 1, 2, 3, or 4;
each instance of r, where present, is an integer selected from 0, 1, 2, or 3;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl;
wherein R¹² may be connected to a=N— ring member to provide an ammonium cation.

Provided herein as Embodiment 165 is the compound or salt of any one of Embodiments 1 to 106 and 146, wherein R² is:

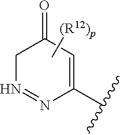

where
each instance of p, where present, is an integer selected from 0, 1, 2, 3, or 4;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl. In several embodiments, a —N=ring nitrogen can be bonded to an R¹² to provide a cation. In several embodiments, no —N=ring nitrogen is bonded to an R¹² to provide a cation.

Provided herein as Embodiment 166 is the compound or salt of any one of Embodiments 1 to 106 and 146, wherein R² is:

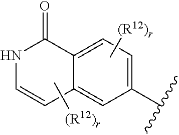

where
each instance of r, where present, is an integer selected from 0, 1, 2, or 3;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl.

Provided herein as Embodiment 167 is the compound or salt of any one of Embodiments 1 to 106 and 146, wherein R² is

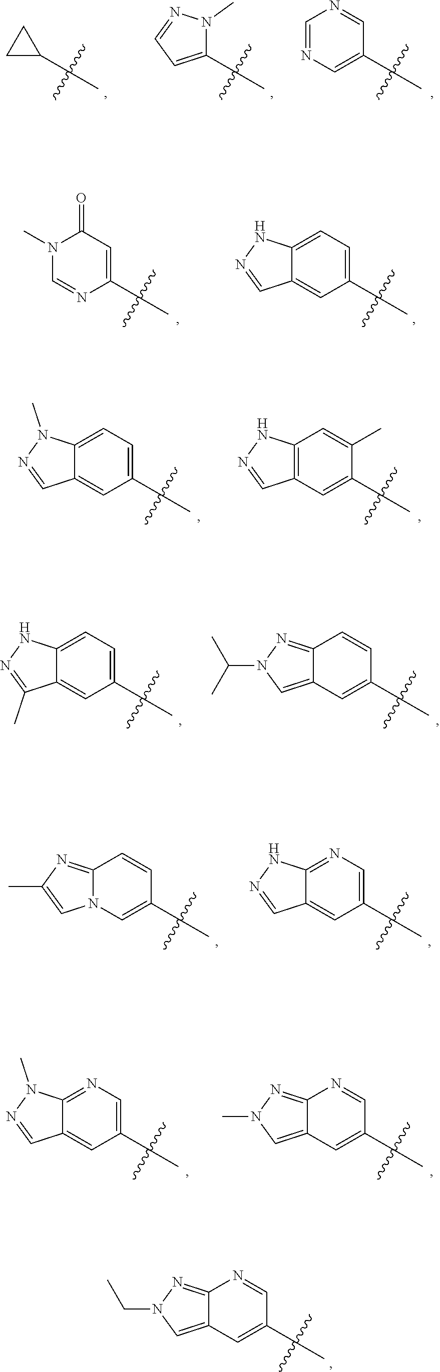

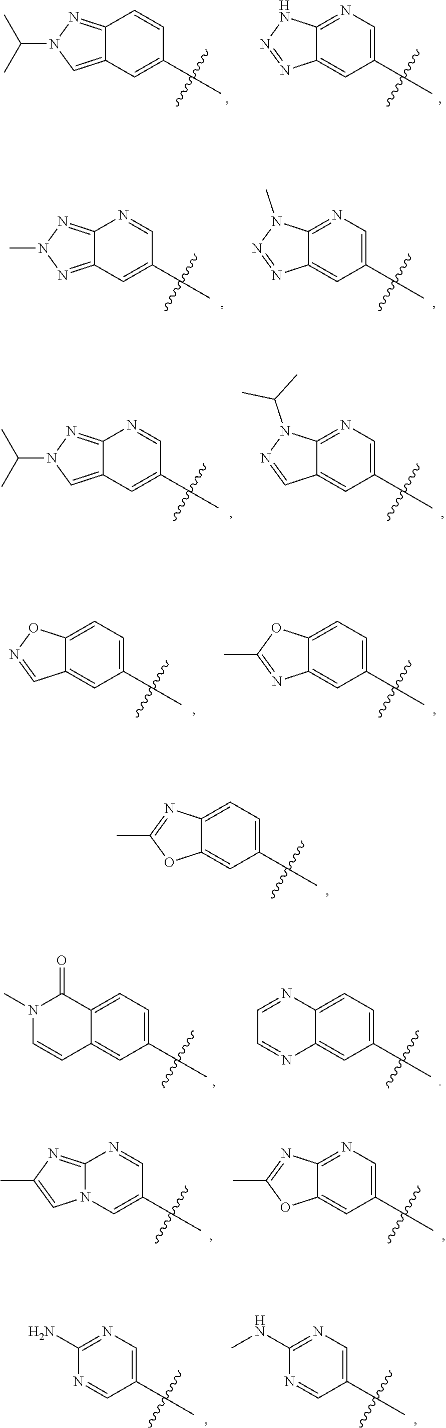

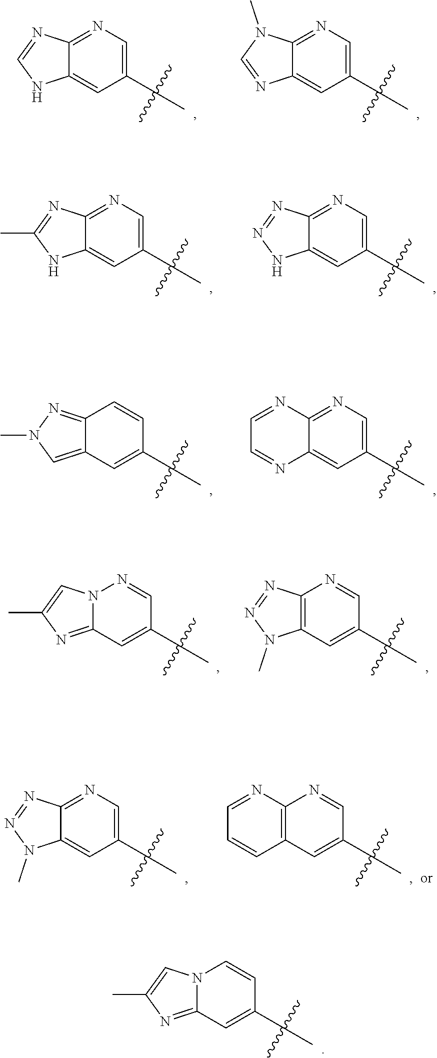

For example, in several embodiments, R² (including its substituents) is

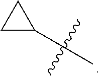 [SMILES: *C1CC1]

In several embodiments, R² (including its substituents) is

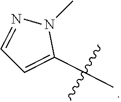 [SMILES: *c1ccnn1C]

In several embodiments, R² (including its substituents) is

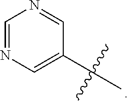 [SMILES: *c1cncnc1]

In several embodiments, R² (including its substituents) is

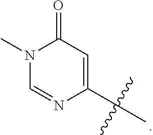 [SMILES: *c1cc(=O)n(C)cn1]

In several embodiments, R² (including its substituents) is

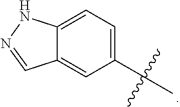 [SMILES: *c1ccc2[nH]ncc2c1]

In several embodiments, R² (including its substituents) is

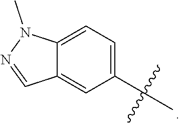 [SMILES: *c1ccc2c(cnn2C)c1]

In several embodiments, R² (including its substituents) is

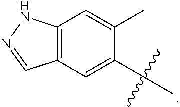 [SMILES: *c1cc2cn[nH]c2cc1C]

In several embodiments, R² (including its substituents) is

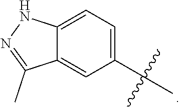 [SMILES: *c1ccc2[nH]nc(C)c2c1]

In several embodiments, R² (including its substituents) is

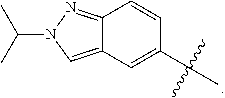 [SMILES: *c1ccc2nn(C(C)C)cc2c1]

In several embodiments, R² (including its substituents) is

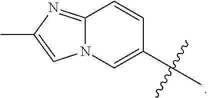 [SMILES: *c1ccc2nc(C)cn2c1]

In several embodiments, R² (including its substituents) is

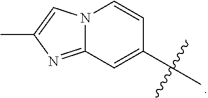 [SMILES: *c1ccn2cc(C)nc2c1]

In several embodiments, R² (including its substituents) is

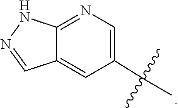 [SMILES: *c1cnc2[nH]ncc2c1]

In several embodiments, R² (including its substituents) is

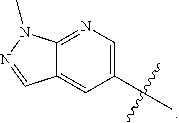 [SMILES: *c1cnc2c(cnn2C)c1]

In several embodiments, R² (including its substituents) is

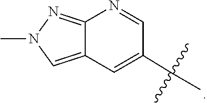 [SMILES: *c1cnc2nn(C)cc2c1]

In several embodiments, R² (including its substituents) is

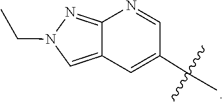 [SMILES: *c1cnc2nn(CC)cc2c1]

In several embodiments, R² (including its substituents) is

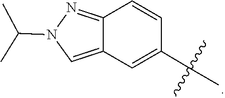 [SMILES: *c1ccc2nn(C(C)C)cc2c1]

In several embodiments, R² (including its substituents) is

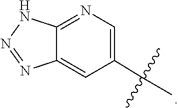 [SMILES: *c1cnc2[nH]nnc2c1]

In several embodiments, R² (including its substituents) is

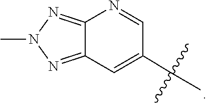 [SMILES: *c1cnc2nn(C)nc2c1]

In several embodiments, R² (including its substituents) is

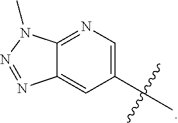

In several embodiments, R² (including its substituents) is

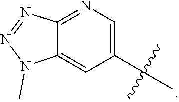

In several embodiments R² (including its substituents) is

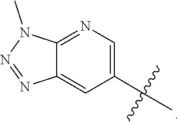 [SMILES: *c1cnc2c(c1)nnn2C]

In several embodiments R² (including its substituents) is

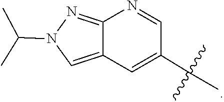 [SMILES: *c1cnc2nn(C(C)C)cc2c1]

In several embodiments, R² (including its substituents) is

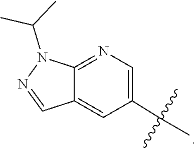 [SMILES: *c1cnc2c(cnn2C(C)C)c1]

In several embodiments, R² (including its substituents) is

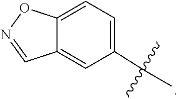 [SMILES: *c1ccc2oncc2c1]

In several embodiments, R² (including its substituents) is

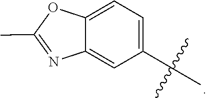 [SMILES: *c1ccc2oc(C)nc2c1]

In several embodiments, R² (including its substituents) is

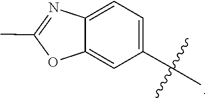 [SMILES: *c1ccc2nc(C)oc2c1]

In several embodiments, R² (including its substituents) is

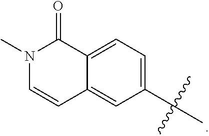 [SMILES: *c1ccc2c(=O)n(C)ccc2c1]

In several embodiments, R² (including its substituents) is

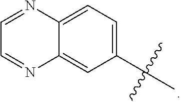 [SMILES: *c1ccc2nccnc2c1]

In several embodiments, R² (including its substituents) is

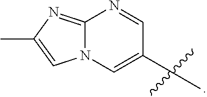 [SMILES: *c1cnc2nc(C)cn2c1]

In several embodiments, R² (including its substituents) is

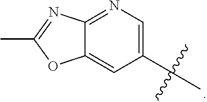 [SMILES: *c1cnc2nc(C)oc2c1]

In several embodiments, R² (including its substituents) is

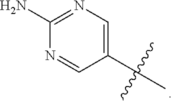 [SMILES: *c1cnc(N)nc1]

In several embodiments, R² (including its substituents) is

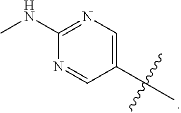 [SMILES: *c1cnc(NC)nc1]

In several embodiments, R² (including its substituents) is

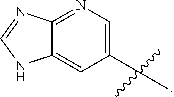 [SMILES: *c1cnc2nc[nH]c2c1]

In several embodiments, R² (including its substituents) is

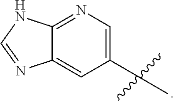 [SMILES: *c1cnc2[nH]cnc2c1]

In several embodiments, RN (including its substituents) is

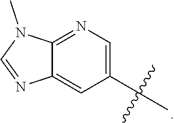 [SMILES: *c1cnc2c(c1)ncn2C]

In several embodiments, R² (including its substituents) is

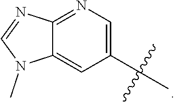 [SMILES: *c1cnc2ncn(C)c2c1]

In several embodiments, R² (including its substituents) is

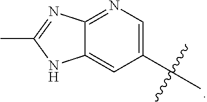 [SMILES: *c1cnc2nc(C)[nH]c2c1]

In several embodiments, R (including its substituents) is

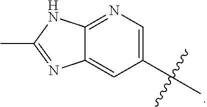 [SMILES: *c1cnc2[nH]c(C)nc2c1]

In several embodiments, R² (including its substituents) is

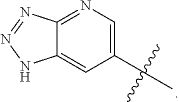 [SMILES: *c1cnc2nn[nH]c2c1]

In several embodiments, R² (including its substituents) is

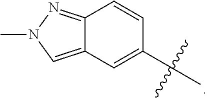 [SMILES: *c1ccc2nn(C)cc2c1]

In several embodiments, R² (including its substituents) is

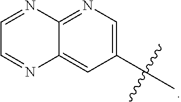 [SMILES: *c1cnc2nccnc2c1]

In several embodiments, R² (including its substituents is

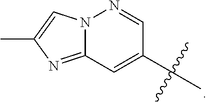 [SMILES: *c1cnn2cc(C)nc2c1]

In several embodiments, R² including its substituents) is

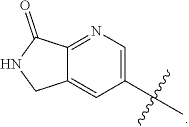 [SMILES: *c1cnc2c(c1)CNC2=O]

In several embodiments. R² (including its substituents) is

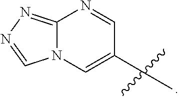 [SMILES: *c1cnc2nncn2c1]

In several embodiments, R² (including its substituents) is

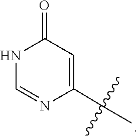 [SMILES: *c1cc(=O)[nH]cn1]

In several embodiments, R² (including its substituents) is

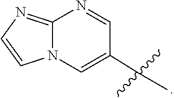 [SMILES: *c1cnc2nccn2c1]

In several embodiments, R² (including its substituents) is

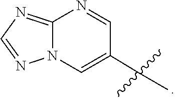 [SMILES: *c1cnc2ncnn2c1]

In several embodiments, R² (including its substituents) is

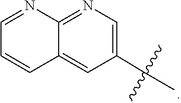 [SMILES: *c1cnc2ncccc2c1]

In several embodiments, R² (including its substituents) is

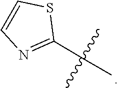 [SMILES: *c1nccs1]

In several embodiments, R² (including its substituents) is

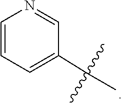 [SMILES: *c1cccnc1]

In several embodiments, R² (including its substituents) is

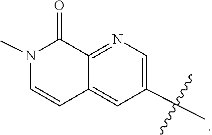 [SMILES: *c1cnc2c(=O)n(C)ccc2c1]

In several embodiments, R² (including its substituents) is

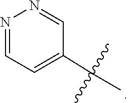 [SMILES: *c1ccnnc1]

In several embodiments, R² (including its substituents) is

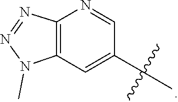

In several embodiments, R² (including its substituents) is

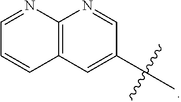 [SMILES: *c1cnc2ncccc2c1]

Any combination of the foregoing R² substituents is envisioned.

Provided herein as Embodiment 168 is the compound or salt of any one of Embodiments 1 to 106 and 146, wherein R² is

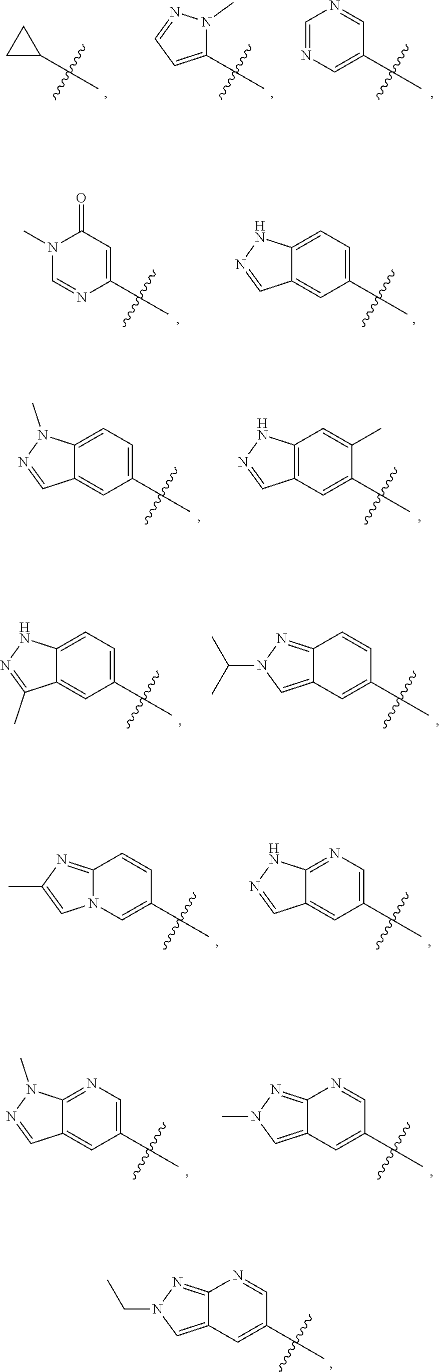

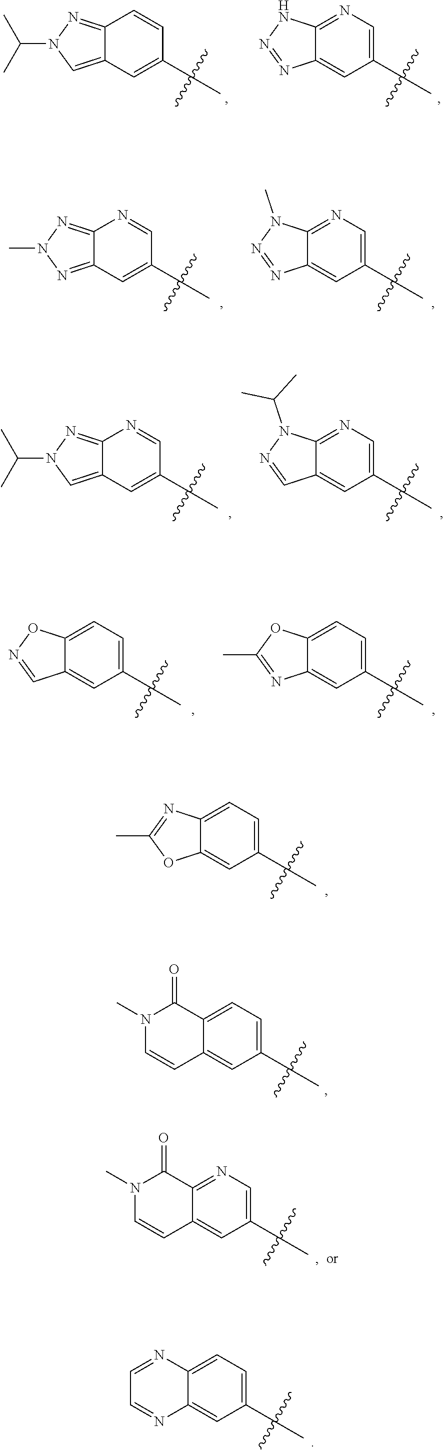

Provided herein as Embodiment 169 is the compound or salt of any one of Embodiments 1 to 20 and 22 to 168, wherein R³ is C₃₋₆cycloalkyl, C₃₋₁₀cycloalkenyl, C₆₋₁₀aryl, heteroaryl having 5 to 10 ring members, or heterocyclyl having 5 to 10 ring members. For example, in several embodiments, R³ is C₃₋₆cycloalkyl. In several embodiments, R³ is a C₄₋₆ cycloalkyl. In several embodiments, R³ is a C₃₋₅ cycloalkyl. In several embodiments, R³ is a C₄₋₆ cycloalkyl. In several embodiments, R³ is a C₃₋₄ cycloalkyl. In several embodiments, R³ is a C₅₋₆ cycloalkyl. In several embodiments, R³ is a C₃ cycloalkyl (e.g., cyclopropyl). In several embodiments, R³ is a C₄ cycloalkyl (e.g., cyclobutyl). In several embodiments, R³ is a C₅ cycloalkyl. In several embodiments, R³ is a C₆ cycloalkyl.

Provided herein as Embodiment 170 is the compound or salt of any one of 1 to 20 and 22 to 169, wherein R³ is unsubstituted or substituted heteroaryl or unsubstituted or substituted heterocyclyl and the R³ heteroaryl or heterocyclyl includes 1, 2, 3, 4, or 5 heteroatom ring members.

Provided herein as Embodiment 171 is the compound or salt of any one of 1 to 20 and 22 to 169, wherein R³ is unsubstituted or substituted heteroaryl or unsubstituted or substituted heterocyclyl and the R³ heteroaryl or heterocyclyl includes 1, 2, 3, or 4 heteroatom ring members.

Provided herein as Embodiment 172 is the compound or salt of any one of 1 to 20 and 22 to 169, wherein R³ is unsubstituted or substituted heteroaryl or unsubstituted or substituted heterocyclyl and the R³ heteroaryl or heterocyclyl includes 1, 2, or 3 heteroatom ring members.

Provided herein as Embodiment 173 is the compound or salt of any one of 1 to 20 and 22 to 169, wherein R³ is unsubstituted or substituted heteroaryl or unsubstituted or substituted heterocyclyl and the R³ heteroaryl or heterocyclyl includes 1 or 2 heteroatom ring members.

Provided herein as Embodiment 174 is the compound or salt of any one of 1 to 20 and 22 to 169, wherein R³ is unsubstituted or substituted heteroaryl or unsubstituted or substituted heterocyclyl and the R³ heteroaryl or heterocyclyl includes 1 heteroatom ring member.

Provided herein as Embodiment 175 is the compound or salt of any one of Embodiments 1 to 20 and 22 to 174, wherein R³ is unsubstituted or substituted heteroaryl or unsubstituted or substituted heterocyclyl and each heteroatom of the R³ ring independently is nitrogen, oxygen, or —S(O)ₓ—, where z is 0, 1, or 2.

Provided herein as Embodiment 176 is the compound or salt of any one of Embodiments 1 to 20 and 22 to 174, wherein R³ is unsubstituted or substituted heteroaryl or unsubstituted or substituted heterocyclyl and each heteroatom of the R³ ring independently is nitrogen or oxygen.

Provided herein as Embodiment 177 is the compound or salt of any one of Embodiments 1 to 20 and 22 to 174, wherein R³ is unsubstituted or substituted heteroaryl or unsubstituted or substituted heterocyclyl and each heteroatom of the R³ ring is nitrogen.

Provided herein as Embodiment 178 is the compound or salt of any one of Embodiments 1 to 20, 22 to 171 and 175 to 177, wherein R³ is unsubstituted or substituted heteroaryl or unsubstituted or substituted heterocyclyl comprising four nitrogen heteroatom ring atoms.

Provided herein as Embodiment 179 is the compound or salt of any one of Embodiments 1 to 20, 22 to 172, and 175 to 177, wherein R³ is unsubstituted or substituted heteroaryl or unsubstituted or substituted heterocyclyl comprising three nitrogen heteroatoms.

Provided herein as Embodiment 180 is the compound or salt of any one of Embodiments 1 to 20, 22 to 173, and 175 to 177, wherein R³ is unsubstituted or substituted heteroaryl or unsubstituted or substituted heterocyclyl comprising two nitrogen heteroatoms.

Provided herein as Embodiment 181 is the compound or salt of any one of Embodiments 1 to 20 and 22 to 177, wherein R³ is unsubstituted or substituted heteroaryl or unsubstituted or substituted heterocyclyl comprising one nitrogen heteroatom.

Provided herein as Embodiment 182 is the compound or salt of any one of Embodiments 1 to 20 and 22 to 176, wherein R³ is unsubstituted or substituted heteroaryl or unsubstituted or substituted heterocyclyl and each heteroatom of the R³ ring is oxygen.

Provided herein as Embodiment 183 is the compound or salt of any one of Embodiments 1 to 20 and 22 to 182, wherein R³ is unsubstituted or substituted fused bicyclic heteroaryl or unsubstituted or substituted fused bicyclic heterocyclic.

Provided herein as Embodiment 184 is the compound or salt of any one of Embodiments 1 to 20 and 22 to 182, wherein R³ is unsubstituted or substituted heteroaryl or unsubstituted or substituted heterocyclyl comprising 5 to 9 ring-members.

Provided herein as Embodiment 185 is the compound or salt of any one of Embodiments 1 to 20 and 22 to 182, wherein R³ is unsubstituted or substituted heteroaryl or unsubstituted or substituted heterocyclyl comprising 5 to 10 ring-members.

Provided herein as Embodiment 186 is the compound or salt of any one of Embodiments 1 to 20 and 22 to 183, wherein R³ is unsubstituted or substituted heteroaryl comprising 8 to 10 ring-members or unsubstituted or substituted heterocyclyl comprising 8 to 10 ring-members.

Provided herein as Embodiment 187 is the compound or salt of any one of Embodiments 1 to 20 and 22 to 183, wherein R³ is unsubstituted or substituted heteroaryl comprising 9 to 10 ring-members or unsubstituted or substituted heterocyclyl comprising 9 to 10 ring-members.

Provided herein as Embodiment 188 is the compound or salt of any one of Embodiments 1 to 20 and 22 to 183, wherein R³ is unsubstituted or substituted heteroaryl comprising 9 ring-members or unsubstituted or substituted heterocyclyl comprising 9 ring-members.

Provided herein as Embodiment 189 is the compound or salt of any one of Embodiments 1 to 20 and 22 to 183, wherein R³ is unsubstituted or substituted heteroaryl comprising 10 ring-members or unsubstituted or substituted heterocyclyl comprising 10 ring-members.

Provided herein as Embodiment 190 is the compound or salt of any one of Embodiments 1 to 20, 22 to 169, and 171 to 182, wherein R³ is unsubstituted or substituted heteroaryl comprising 6 ring-members or unsubstituted or substituted heterocyclyl comprising 6 ring-members.

Provided herein as Embodiment 191 is the compound or salt of any one of Embodiments 1 to 20, 22 to 169, and 171 to 182, wherein R³ is unsubstituted or substituted heteroaryl comprising 5 ring members or unsubstituted or substituted heterocyclyl comprising 5 ring members.

Provided herein as Embodiment 192 is the compound or salt of any one of Embodiments 1 to 20 and 22 to 191, wherein R³ is unsubstituted or substituted heteroaryl.

Provided herein as Embodiment 193 is the compound or salt of any one of Embodiments 1 to 20 and 22 to 191, wherein R³ is unsubstituted or substituted heterocyclyl.

Provided herein as another embodiment is the compound or salt of any one of Embodiments 1 to 20 and 22 to 168, wherein R³ is unsubstituted or substituted C₃₋₄cycloalkyl.

Provided herein as Embodiment 194 is the compound or salt of any one of Embodiments 1 to 20 and 22 to 168, wherein R³ is unsubstituted or substituted C₃₋₄cycloalkyl. For example, in several embodiments, R³ is cyclopropyl. In several embodiments, R³ is cyclobutyl.

Provided herein as Embodiment 195 is the compound or salt of any one of Embodiments 1 to 20 and 22 to 168, wherein R³ is unsubstituted or substituted C₁₋₆alkyl.

Provided herein as Embodiment 196 is the compound or salt of any one of Embodiments 1 to 20 and 22 to 168, wherein R³ is unsubstituted or substituted C₁₋₁₀aryl.

Provided herein as Embodiment 197 is the compound or salt of any one of Embodiments 1 to 20 and 22 to 196, wherein R³ is substituted with 1, 2, 3, 4, or 5 substituents.

Provided herein as Embodiment 198 is the compound or salt of any one of Embodiments 1 to 20 and 22 to 197, wherein R³ is substituted with 1, 2, 3, or 4 substituents.

Provided herein as Embodiment 199 is the compound or salt of any one of Embodiments 1 to 20 and 22 to 198, wherein R³ is substituted with 1, 2, or 3 substituents.

Provided herein as Embodiment 200 is the compound or salt of any one of Embodiments 1 to 20 and 22 to 199, wherein R³ is substituted with 1 or 2 substituents.

Provided herein as Embodiment 201 is the compound or salt of any one of Embodiments 1 to 20 and 22 to 200, wherein R³ is substituted with 1 substituent. For example, in several embodiments, R³

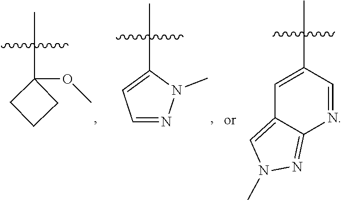

In several embodiments, R³ together with its substituents form alkyl-pyrazolo[3,4-b]pyridine (e.g., 2-methyl-2H-pyrazolo[3,4-b]pyridinyl, etc.).

Provided herein as Embodiment 202 is the compound or salt of any one of Embodiments 1 to 20 and 22 to 201, wherein, when R³ is substituted, each R³ substituent independently is C₁₋₃alkyl, C₁₋₃alkoxy, or C₁₋₃ alkylene-C₁₋₃ alkoxy. For example, in several embodiments, when R³ is substituted, an R³ substituent may be —CH₂OCH₃.

Provided herein as Embodiment 203 is the compound or salt of any one of Embodiments 1 to 20 and 22 to 196, wherein, when R³ is substituted, each R³ substituent independently is methyl, ethyl, or isopropyl.

Provided herein as Embodiment 204 is the compound or salt of any one of Embodiments 1 to 20 and 22 to 203, wherein, when R³ is substituted, R³ is substituted with methyl.

Provided herein as Embodiment 205 is the compound or salt of any one of Embodiments 1 to 20 and 22 to 203, wherein, when R³ is substituted, R³ is substituted with ethyl.

Provided herein as Embodiment 206 is the compound or salt of any one of Embodiments 1 to 20 and 22 to 203, wherein, when R³ is substituted, R³ is substituted with isopropyl.

Provided herein as Embodiment 207 is the compound or salt of any one of Embodiments 1 to 20 and 22 to 196, wherein R³ is unsubstituted.

Provided herein as Embodiment 208 is the compound or salt of any one of Embodiments 1 to 20 and 22 to 168, wherein R³ is —H.

Provided herein as Embodiment 209 is the compound or salt of any one of Embodiments 1 to 20 and 22 to 168, wherein R³ is

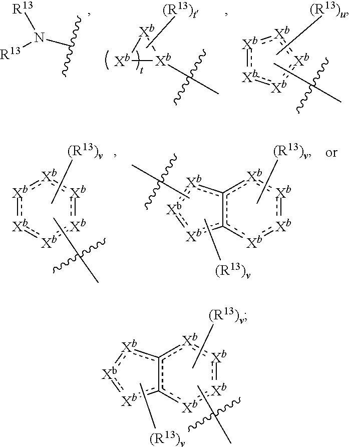

where
“ [symbol] ” indicates a double bond or single bond may be present;
each instance of X^{b} independently is nitrogen, oxygen, or carbon;
each instance of t, where present, is an integer selected from 1, 2, 3, 4, or 5;
each instance of t′, where present, is an integer selected from 1, 2, 3, 4, or 5;
each instance of v, where present, is an integer selected from 0, 1, 2, 3, 4, or 5, wherein where two instances of v occur on the same R³ group, the total of both v values combined does not exceed 5;
each instance of R¹³, when present and attached to an N ring member, independently is C₁₋₃alkyl, C₁₋₃haloalkyl, or C₁₋₃ alkylene-C₁₋₃ alkoxy; and
each instance of R¹³, when present and attached to a C ring member, independently is halogen, —OH, oxo, —N(R^{b})₂, C₁₋₃alkyl, C₁₋₃haloalkyl, or C₁₋₃alkylene-O—C₁₋₃alkyl;
or, alternatively, two instances of R¹³ together form a C₃₋₆ cycloalkyl or heterocyclyl having 3 to 6 ring members, which may provided a fused ring system or spiro ring system when R³ is a ring structure. In several embodiments, a —N=ring nitrogen can be bonded to an R¹³ to provide a cation. In several embodiments, no —N=ring nitrogen is bonded to an R¹³ to provide a cation.

Provided herein as Embodiment 210 is the compound or salt of any one of Embodiments 1 to 20 and 22 to 168, wherein R³ is:

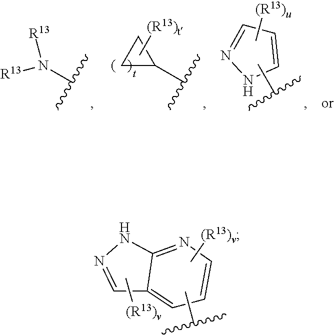

where
each instance of t, where present, is an integer selected from 1, 2, 3, 4, or 5;
each instance of t′, where present, is an integer selected from 1, 2, 3, 4, or 5;
each instance of v, where present, is an integer selected from 0, 1, 2, 3, 4, or 5, wherein where two instances of v occur on the same R³ group, the total of both v values combined does not exceed 5;
each instance of R¹³, when present and attached to an N ring member, independently is C₁₋₃alkyl, C₁₋₃haloalkyl, or C₁₋₃ alkylene-C₁₋₃ alkoxy; and
each instance of R¹³, when present and attached to a C ring member, independently is halogen, —OH, oxo, C₁₋₃alkyl, C₁₋₃haloalkyl, or C₁₋₃alkylene-O—C₁₋₃alkyl;
or, alternatively, two instances of R¹³ together form a C₃₋₆ cycloalkyl or heterocyclyl having 3 to 6 ring members, which may provide a fused ring system or spiro ring system when R³ is a ring structure. In several embodiments, a —N=ring nitrogen can be bonded to an R¹³ to provide a cation. In several embodiments, no —N=ring nitrogen is bonded to an R¹³ to provide a cation.

Provided herein as Embodiment 211 is the compound or salt of any one of Embodiments 1 to 20 and 22 to 168, wherein R³ together with its substituents is

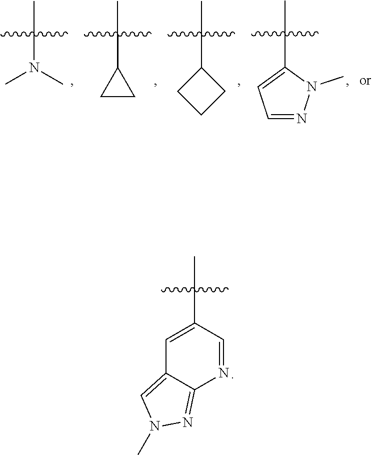

Provided herein as Embodiment 212 is the compound or salt of any one of Embodiments 1 to 20 and 22 to 168, wherein R³ is:

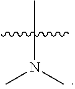

Provided herein as Embodiment 213 is the compound or salt of any one of Embodiments 1 to 20 and 22 to 168, wherein R³ is:

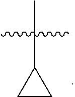 [SMILES: *C1CC1]

Provided herein as Embodiment 214 is the compound or salt of any one of Embodiments 1 to 20 and 22 to 168, wherein R³ is:

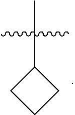 [SMILES: *C1CCC1]

Provided herein as Embodiment 215 is the compound or salt of any one of Embodiments 1 to 20 and 22 to 168, wherein R³ is:

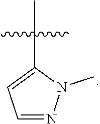 [SMILES: *c1ccnn1C]

Provided herein as Embodiment 216 is the compound or salt of any one of Embodiments 1 to 20 and 22 to 168, wherein R³ is:

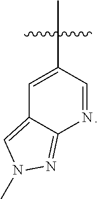 [SMILES: *c1cnc2nn(C)cc2c1]

Provided herein as Embodiment 217 is the compound or salt of any one of Embodiments 1 to 216, wherein each instance of R independently is substituted with 1, 2, 3, 4, or 5 substituents.

Provided herein as Embodiment 218 is the compound or salt of any one of Embodiments 1 to 217, wherein each instance of R independently is substituted with 1, 2, 3, or 4 substituents.

Provided herein as Embodiment 219 is the compound or salt of any one of Embodiments 1 to 218, wherein each instance of R independently is substituted with 1, 2, or 3 substituents.

Provided herein as Embodiment 220 is the compound or salt of any one of Embodiments 1 to 219, wherein each instance of R independently is substituted with 1 or 2 substituents.

Provided herein as Embodiment 221 is the compound or salt of any one of Embodiments 1 to 220, wherein each instance of R independently is substituted with 1 substituent.

Provided herein as Embodiment 222 is the compound or salt of any one of Embodiments 1 to 230, wherein each instance of R is unsubstituted.

Provided herein as Embodiment 223 is the compound or salt of any one of Embodiments 1 to 24 and 26 to 222, wherein R⁴ is —H, C₁₋₃ alkyl, halogen, or —N(R^{a})₂ where each instance of R^{a}, where present, independently is —H or C₁₋₆ alkyl.

Provided herein as Embodiment 224 is the compound or salt of any one of Embodiments 1 to 24 and 26 to 223, is the compound or salt of any one of Embodiments 1 to 24 and 26 to 222, wherein R⁴ is —N(R^{a})₂.

Provided herein as Embodiment 225 is the compound or salt of any one of Embodiments 1 to 24 and 26 to 224, wherein each instance of R independently is substituted with 1, 2, 3, 4, or 5 substituents.

Provided herein as Embodiment 226 is the compound or salt of any one of Embodiments 1 to 24 and 26 to 224, wherein each instance of R independently is substituted with 1, 2, 3, or 4 substituents.

Provided herein as Embodiment 227 is the compound or salt of any one of Embodiments 1 to 24 and 26 to 224, wherein each instance of R independently is substituted with 1, 2, or 3 substituents.

Provided herein as Embodiment 228 is the compound or salt of any one of Embodiments 1 to 24 and 26 to 224, wherein each instance of R independently is substituted with 1 or 2 substituents.

Provided herein as Embodiment 229 is the compound or salt of any one of Embodiments 1 to 24 and 26 to 224, wherein each instance of R independently is substituted with 1 substituent.

Provided herein as Embodiment 230 is the compound or salt of any one of Embodiments 1 to 24 and 26 to 224, wherein each instance of R is unsubstituted.

Provided herein as Embodiment 231 is the compound or salt of any one of Embodiments 1 to 24 and 26 to 230, wherein at least one instance of R is C₁₋₃alkyl.

Provided herein as Embodiment 232 is the compound or salt of any one of Embodiments 1 to 24, 26 to 223, and 231, wherein at least one instance of R is —CH₃.

Provided herein as Embodiment 233 is the compound or salt of any one of Embodiments 1 to 24, 26 to 223, and 231 to 232, wherein at least one instance of R is —H.

Provided herein as Embodiment 234 is the compound or salt of any one of Embodiments 1 to 24, 26 to 223, and 231 to 233, wherein each instance of R is —H.

Provided herein as Embodiment 235 is the compound or salt of any one of Embodiments 1 to 24 and 26 to 223, wherein R⁴ is C₁₋₃ alkyl.

Provided herein as Embodiment 236 is the compound or salt of any one of Embodiments 1 to 24 and 26 to 223, wherein R⁴ is methyl.

Provided herein as Embodiment 237 is the compound or salt of any one of Embodiments 1 to 24 and 26 to 223, wherein R⁴ is halogen.

Provided herein as Embodiment 238 is the compound or salt of any one of Embodiments 1 to 24 and 26 to 223, wherein R⁴ is —Cl.

Provided herein as Embodiment 239 is the compound or salt of any one of Embodiments 1 to 24 and 26 to 223, wherein R⁴ is —H.

Provided herein as Embodiment 240 is the compound or salt of any one of Embodiments 1 to 239, wherein R independently is substituted with 1, 2, 3, or 4 substituents. For example, in several embodiments, R⁵ is substituted with 1 to 4 substituents. In several embodiments, R⁵ is substituted with 1 to 3 substituents. In several embodiments, R⁵ is substituted with 1 to 2 substituents. In several embodiments, R⁵ is substituted with 2 to 4 substituents. In several embodiments, R⁵ is substituted with 3 to 4 substituents. In several embodiments, R⁵ is substituted with 2 to 3 substituents. In several embodiments, R⁵ is substituted with 1, 2, or 4 substituents.

Provided herein as Embodiment 241 is the compound or salt of any one of Embodiments 1 to 22 and 24 to 239, wherein R⁵ independently is substituted with 1, 2, or 3 substituents.

Provided herein as Embodiment 242 is the compound or salt of any one of Embodiments 1 to 22 and 24 to 239, wherein R⁵ independently is substituted with 1 or 2 substituents.

Provided herein as Embodiment 243 is the compound or salt of any one of Embodiments 1 to 22 and 24 to 239, wherein R⁵ independently is substituted with 1 substituent.

Provided herein as Embodiment 244 is the compound or salt of any one of Embodiments 1 to 22 and 24 to 239, wherein R⁵ is unsubstituted.

Provided herein as Embodiment 245 is the compound or salt of any one of Embodiments 1 to 22 and 24 to 244, wherein R is —H or unsubstituted or substituted C₁₋₆ alkyl.

Provided herein as Embodiment 246 is the compound or salt of any one of Embodiments 1 to 22 and 24 to 239, wherein R⁵ is —H or —CH₂OH.

Provided herein as Embodiment 247 is the compound or salt of any one of Embodiments 1 to 22 and 24 to 239, wherein R⁵ is —H.

Provided herein as Embodiment 248 is the compound or salt of any one of Embodiments 1 to 22 and 24 to 239, wherein R⁵ is —CH₂OCH₃.

Provided herein as Embodiment 249 is the compound or salt of any one of Embodiments 1, 2, 10 to 13, 15 to 20, and 34 to 248, wherein the compound of Formula (A) is a compound of Formula (II):

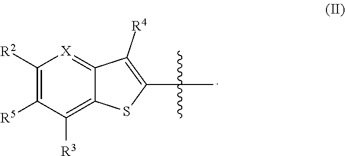

Provided herein as Embodiment 250 is the compound or salt of any one of Embodiments 1 to 3, 5, 8, 10 to 14, 16 to 248, wherein the compound of Formula (A) is a compound of Formula (III):

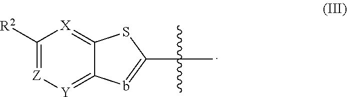

Provided herein as Embodiment 251 is the compound or salt of any one of Embodiments 1, 2, 4, 6 to 13, 15 to 26, and 34 to 248, wherein the compound of Formula (A) is a compound of Formula (IV):

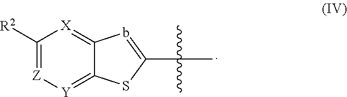

Provided herein as Embodiment 252 is the compound or salt of any one of Embodiments 1, 2, 5, 10 to 14, 16 to 22, 25, 26, and 34 to 239, wherein the compound of Formula (A) is a compound of Formula (V):

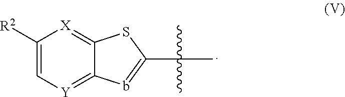

Provided herein as Embodiment 253 is the compound or salt of any one of Embodiments 1, 2, 4, 6, 7, 10 to 13, 15 to 22, 25, 26, and 34 to 239, wherein the compound of Formula (A) is a compound of Formula (VI):

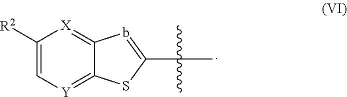

Provided herein as Embodiment 254 is the compound or salt of any one of Embodiments 1 to 5, 8, 10 to 14, 16 to 20, 23 to 26, 34 to 168, and 217 to 248, wherein the compound of Formula (A) is a compound of Formula (VII):

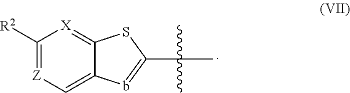

Provided herein as Embodiment 255 is the compound or salt of any one of Embodiments 1, 2, 4, 6 to 13, 15 to 20, 23 to 26, and 34 to 168, and 217 to 248, wherein the compound of Formula (A) is a compound of Formula (VIII):

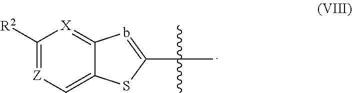

Provided herein as Embodiment 256 is the compound or salt of any one of Embodiments 1, 2, 5, 10 to 14, 16 to 20, 25 to 168, and 217 to 239, wherein the compound of Formula (A) is a compound of Formula (IX):

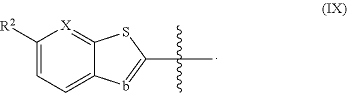

Provided herein as Embodiment 257 is the compound or salt of any one of Embodiments 1, 2, 6, 7, 10 to 13, 15 to 20, 25, 26, 34 to 168, and 217 to 239, wherein the compound of Formula (A) is a compound of Formula (X):

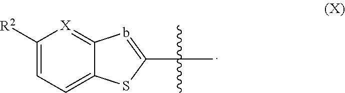

Provided herein as Embodiment 258 is the compound or salt of any one of Embodiments 1, 2, 10 to 12, 16 to 22, and 34 to 222, wherein the compound of Formula (A) is a compound of Formula (XI):

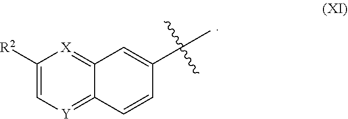

Provided herein as Embodiment 259 is the compound or salt of any one of Embodiments 1, 2, 10 to 12, 16 to 22, and 34 to 222, wherein the compound of Formula (A) is a compound of Formula (XII):

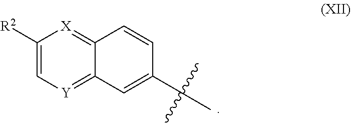

Provided herein as Embodiment 260 is the compound or salt of any one of Embodiments 1, 27 to 97, 107 to 222, and 240 to 248, wherein the compound of Formula (A) is a compound of Formula (IAi):

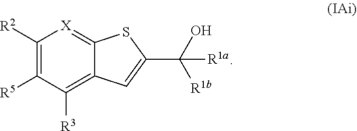

Provided herein as Embodiment 261 is the compound or salt of any one of Embodiments 1, 2, 10-14, 16, 19, 20, 27 to 97, 107 to 222, and 240 to 248, wherein the compound of Formula (A) is a compound of Formula (IAii):

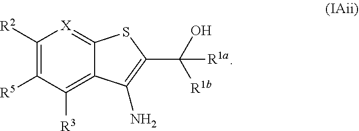

Provided herein as Embodiment 262 is the compound or salt of any one of Embodiments 1, 2, 10-14, 16, 19, 20, 22, 24, 26 to 97, 107 to 239, wherein the compound of Formula (A) is a compound of Formula (IBi):

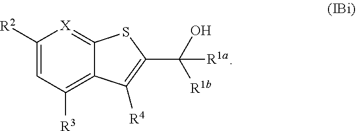

Provided herein as Embodiment 263 is the compound or salt of any one of Embodiments 1, 2, 10-14, 16, 19, 20, 22, 26 to 96, and 107 to 222, wherein the compound of Formula (A) is a compound of Formula (IBii):

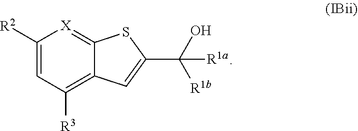

Provided herein as Embodiment 264 is the compound or salt of any one of Embodiments 1, 2, 10-14, 16, 19, 20, 22, 26 to 96, 107 to 222, wherein the compound of Formula (A) is a compound of Formula (IBiii):

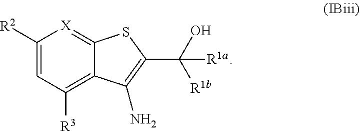

Provided herein as Embodiment 265 is the compound or salt of any one of Embodiments 1, 2, 11-14, 16, 19, 20, 24, 26 to 97, 107 to 168, and 217 to 248, wherein the compound of Formula (A) is a compound of Formula (ICi):

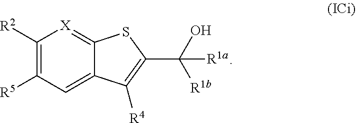

Provided herein as Embodiment 266 is the compound or salt of any one of Embodiments 1, 2, 11-14, 16, 19, 20, 24, 26 to 97, 107 to 168, 217 to 222, and 240 to 248, wherein the compound of Formula (A) is a compound of Formula (ICii):

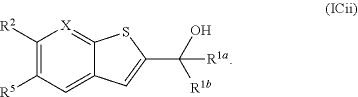

Provided herein as Embodiment 267 is the compound or salt of any one of Embodiments 1, 2, 11-14, 16, 19, 20, 24, 27 to 97,103,107 to 168, 217 to 222, and 240 to 248, wherein the compound of Formula (A) is a compound of Formula (ICiii):

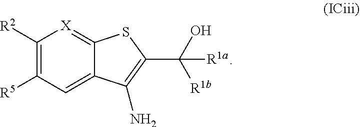

Provided herein as Embodiment 268 is the compound or salt of any one of Embodiments 1, 2, 11-14, 16, 19, 20, 27 to 97, 107 to 168, and 217 to 239, wherein the compound of Formula (A) is a compound of Formula (IDi):

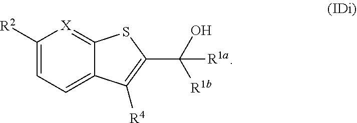

Provided herein as Embodiment 269 is the compound or salt of any one of Embodiments 1, 2, 11-14, 16, 19, 20, 27 to 97, 107 to 168, and 217 to 222, wherein the compound of Formula (A) is a compound of Formula (IDii):

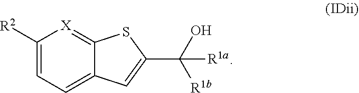

Provided herein as Embodiment 270 is the compound or salt of any one of Embodiments 1, 2, 11-14, 16, 27 to 97, 107 to 168, and 217 to 222, wherein the compound of Formula (A) is a compound of Formula (I-1):

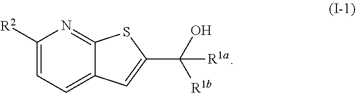

Provided herein as Embodiment 271 is the compound or salt of any one of Embodiments 1, 2, 11-14, 16, 27 to 97, 107 to 168, and 217 to 239, wherein the compound of Formula (A) is a compound of Formula (I-2):

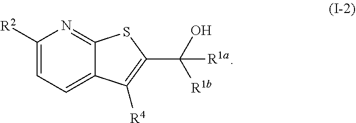

Provided herein as Embodiment 272 is the compound or salt of any one of Embodiments 1, 2, 11-14, 16, 27 to 97, 107 to 168, and 217 to 222, wherein the compound of Formula (A) is a compound of Formula (1-3):

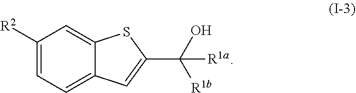

Provided herein as Embodiment 273 is the compound or salt of any one of Embodiments 1, 2, 11-14, 16, 34 to 97, 107 to 168, and 217 to 222, wherein the compound of Formula (A) is a compound of Formula (III-1):

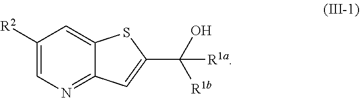

Provided herein as Embodiment 274 is the compound or salt of any one of Embodiments 1, 2, 11-14, 16, 34 to 97, 107 to 168, and 217 to 222, wherein the compound of Formula (A) is a compound of Formula (III-2):

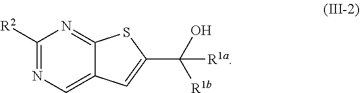

Provided herein as Embodiment 275 is the compound or salt of any one of Embodiments 1, 2, 11-14, 16, 34 to 97, 107 to 168, and 217 to 222, wherein the compound of Formula (A) is a compound of Formula (III-3):

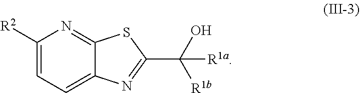

Provided herein as Embodiment 276 is the compound or salt of any one of Embodiments 1, 2, 11-14, 16, 34 to 97, 107 to 168, and 217 to 222, wherein the compound of Formula (A) is a compound of Formula (III-4):

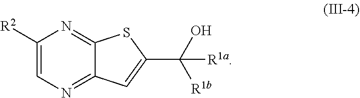

Provided herein as Embodiment 277 is the compound or salt of any one of Embodiments 1, 2, 11-14, 16, 34 to 97, 107 to 168, and 217 to 239, wherein the compound of Formula (A) is a compound of Formula (III-5):

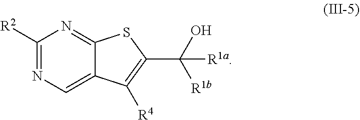

Provided herein as Embodiment 278 is the compound or salt of any one of Embodiments 1, 2, 11-13, 15, 16, 34 to 97, 107 to 168, and 217 to 222, wherein the compound of Formula (A) is a compound of Formula (IV-1):

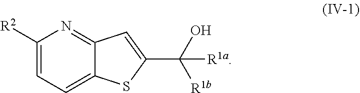

Provided herein as Embodiment 279 is the compound or salt of any one of Embodiments 1, 2, 11-13, 15, 16, 34 to 97, 107 to 168, and 217 to 222, wherein the compound of Formula (A) is a compound of Formula (IV-2):

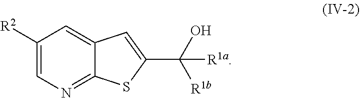

Provided herein as Embodiment 280 is the compound or salt of any one of Embodiments 1, 2, 11-13, 15, 16, 34 to 97, 107 to 168, and 217 to 222, wherein the compound of Formula (A) is a compound of Formula (IV-3):

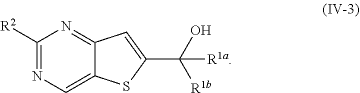

Provided herein as Embodiment 281 is the compound or salt of any one of Embodiments 1, 2, 11-13, 15, 16, 34 to 97, 107 to 168, and 217 to 222, wherein the compound of Formula (A) is a compound of Formula (IV-4):

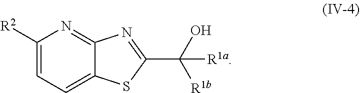

Provided herein as Embodiment 282 is the compound or salt of any one of Embodiments 1, 2, 34 to 97, 107 to 168, and 217 to 222, wherein the compound of Formula (A) is a compound of Formula (XI-1):—

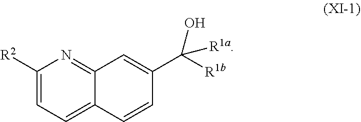

Provided herein as Embodiment 283 is the compound or salt of any one of Embodiments 1, 2, 34 to 97, 107 to 168, and 217 to 222, wherein the compound of Formula (A) is a compound of Formula (XI-2):

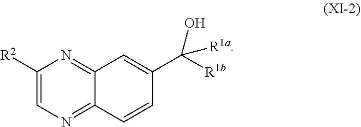

Provided herein as Embodiment 284 is the compound or salt of any one of Embodiments 1, 2, 34 to 97, 107 to 168, and 217 to 222, wherein the compound of Formula (A) is a compound of Formula (XII-1):

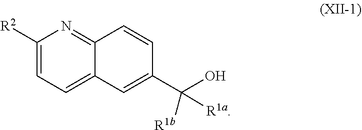

Provided herein as Embodiment 285 is the compound or salt of Embodiment 1 to 284, wherein no more than two of X, Y, and Z are N.

Provided herein as Embodiment 286 is the compound or salt of Embodiment 1 or 16, wherein the compound is:

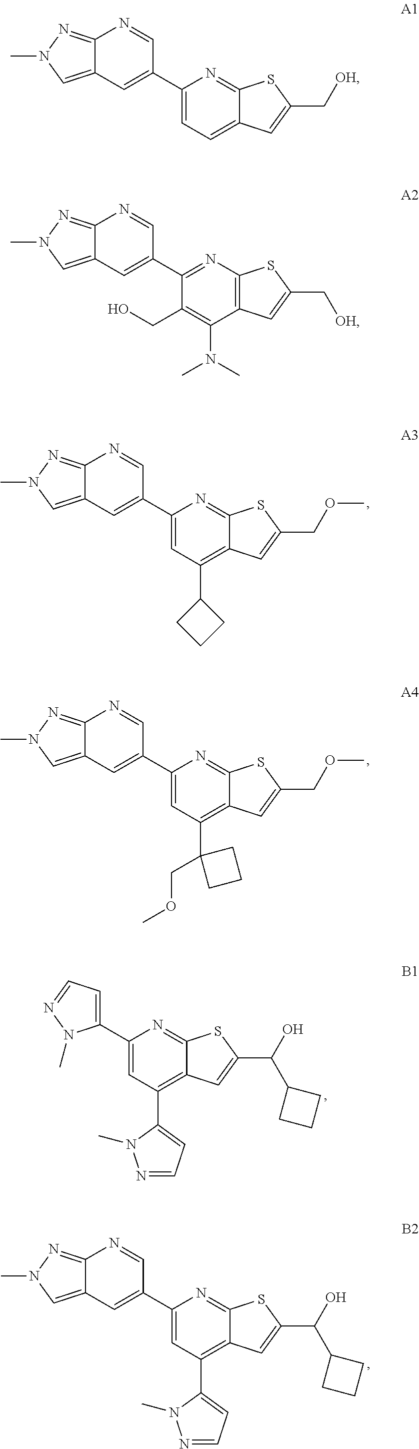

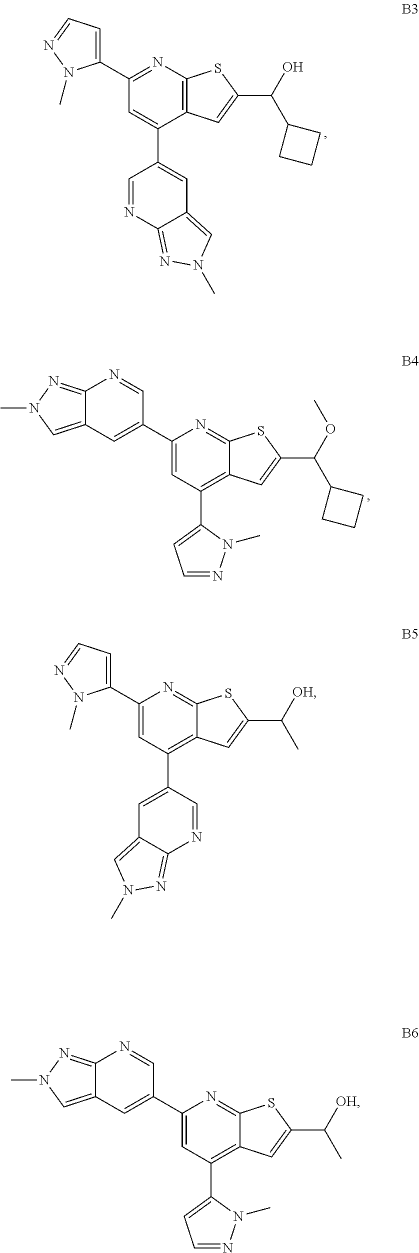

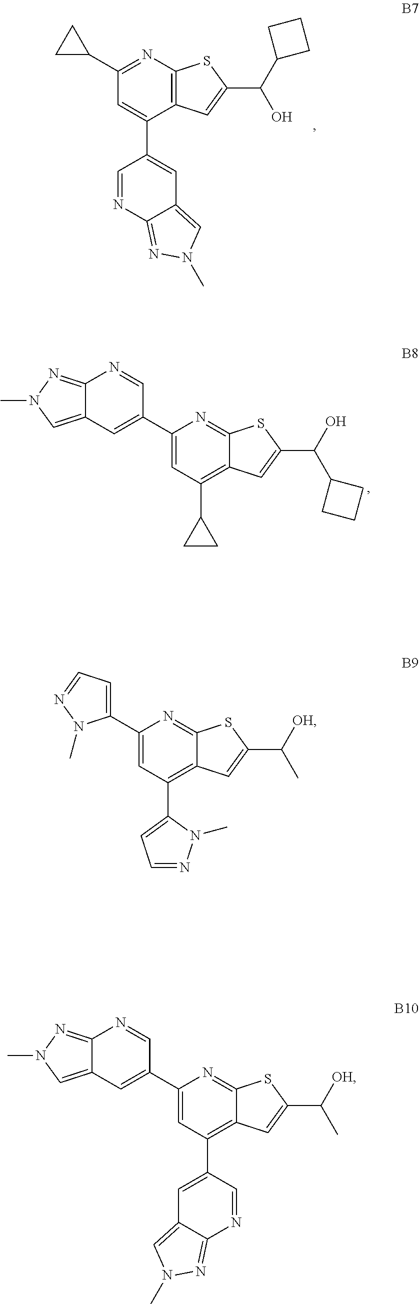

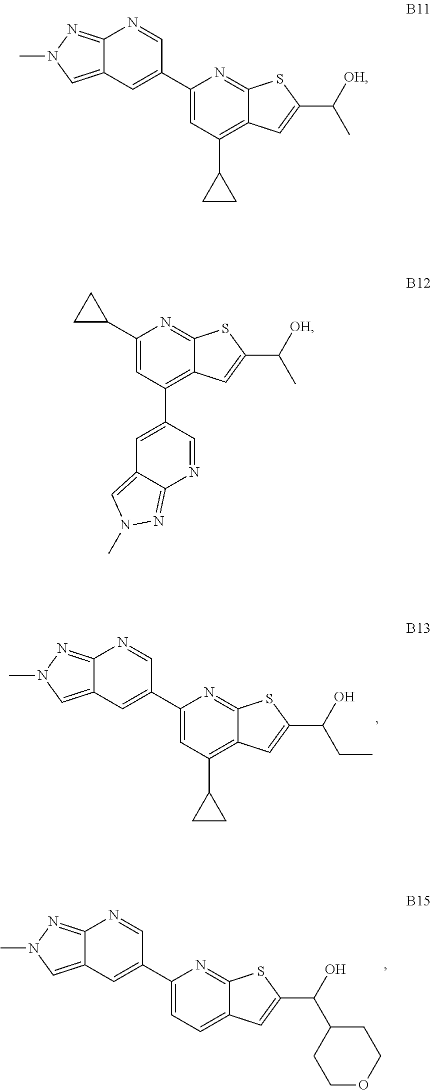

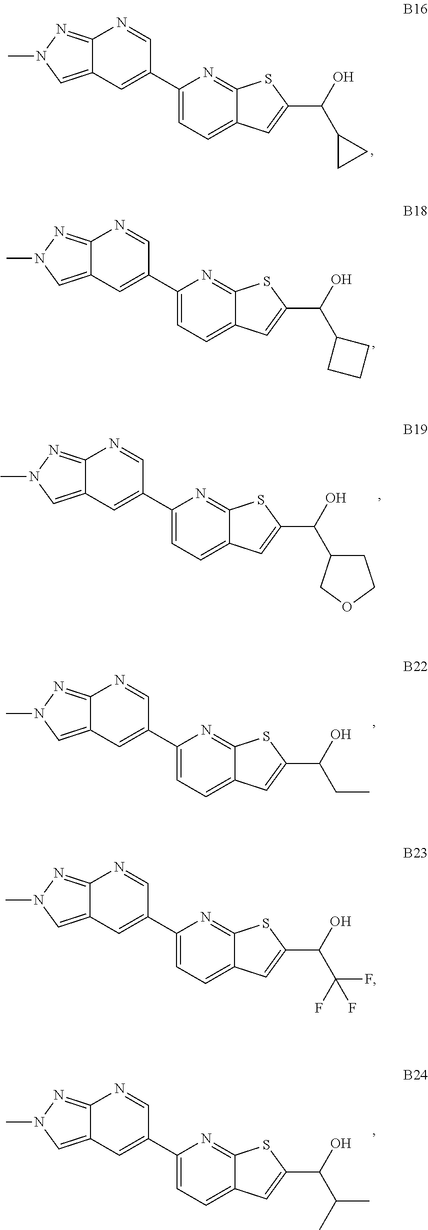

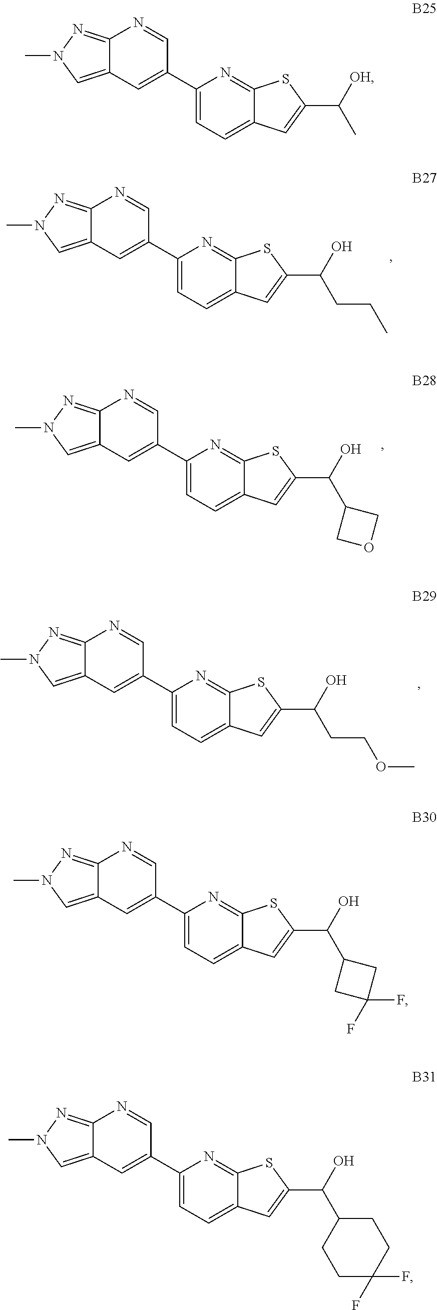

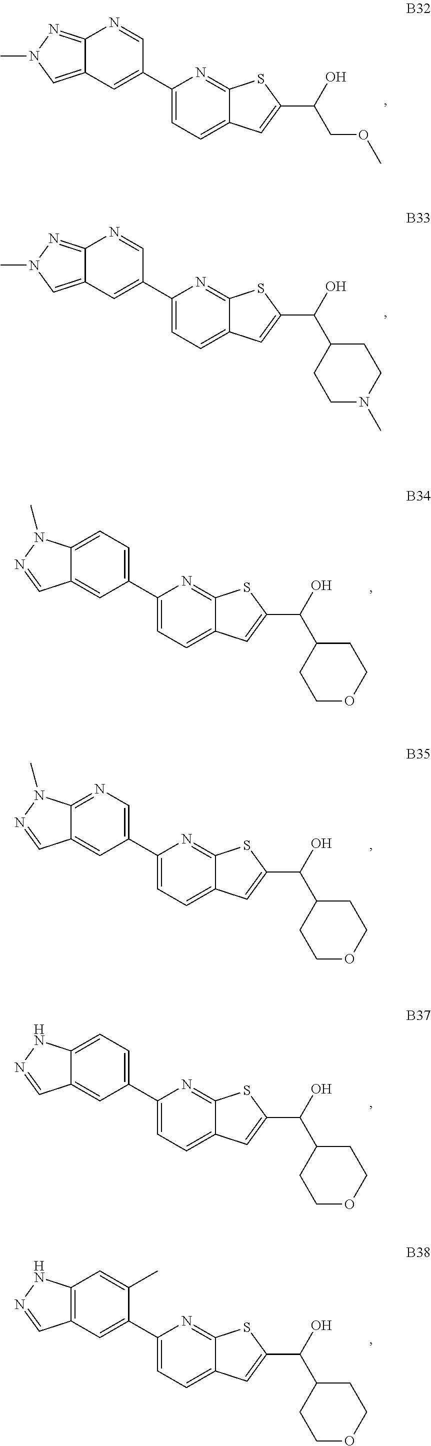

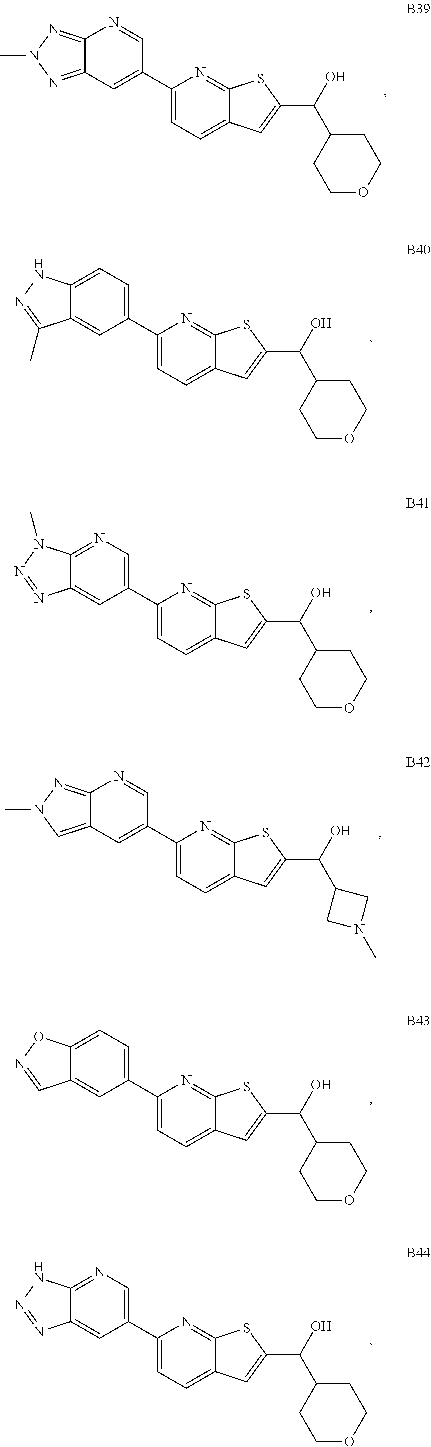

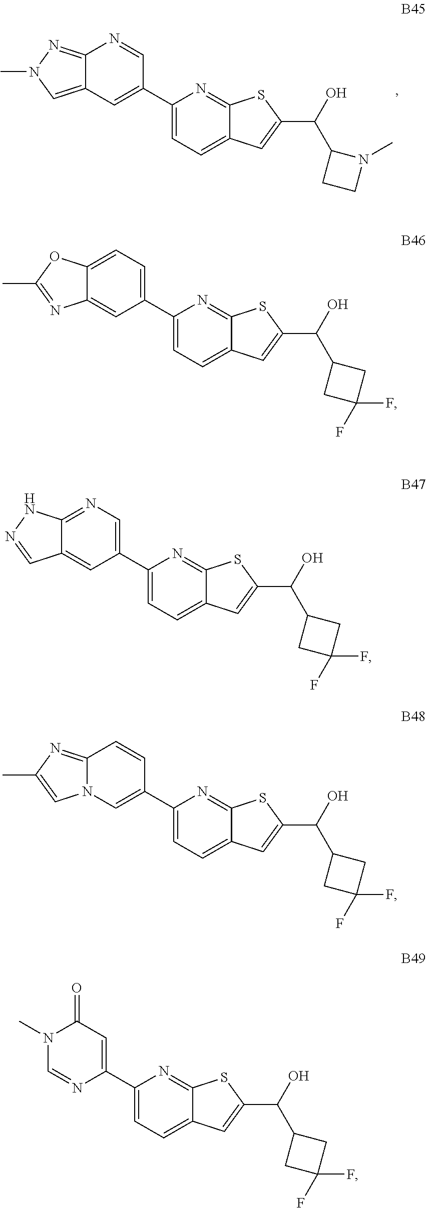

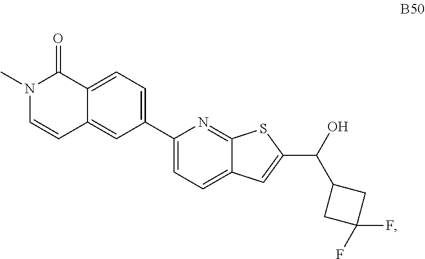 [SMILES: Cn1ccc2cc(-c3ccc4cc(C(O)C5CC(F)(F)C5)sc4n3)ccc2c1=O]

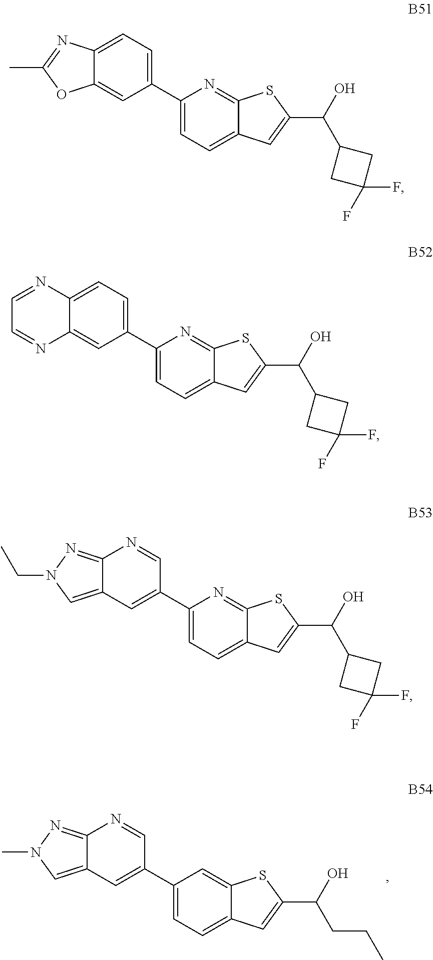 [SMILES: Cc1nc2ccc(-c3ccc4cc(C(O)C5CC(F)(F)C5)sc4n3)cc2o1 ; OC(c1cc2ccc(-c3ccc4nccnc4c3)nc2s1)C1CC(F)(F)C1 ; CCn1cc2cc(-c3ccc4cc(C(O)C5CC(F)(F)C5)sc4n3)cnc2n1 ; CCCC(O)c1cc2ccc(-c3cnc4nn(C)cc4c3)cc2s1]

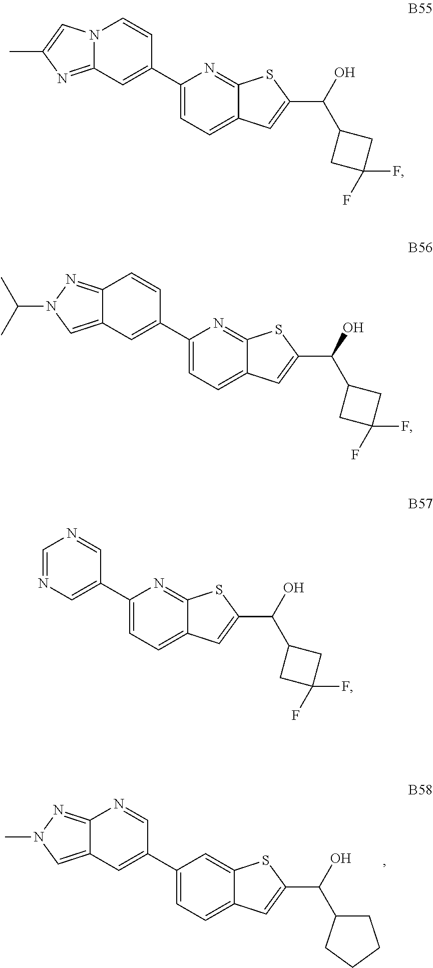 [SMILES: Cc1cn2ccc(-c3ccc4cc(C(O)C5CC(F)(F)C5)sc4n3)cc2n1 ; CC(C)n1cc2cc(-c3ccc4cc([C@@H](O)C5CC(F)(F)C5)sc4n3)ccc2n1 ; OC(c1cc2ccc(-c3cncnc3)nc2s1)C1CC(F)(F)C1 ; Cn1cc2cc(-c3ccc4cc(C(O)C5CCCC5)sc4c3)cnc2n1]

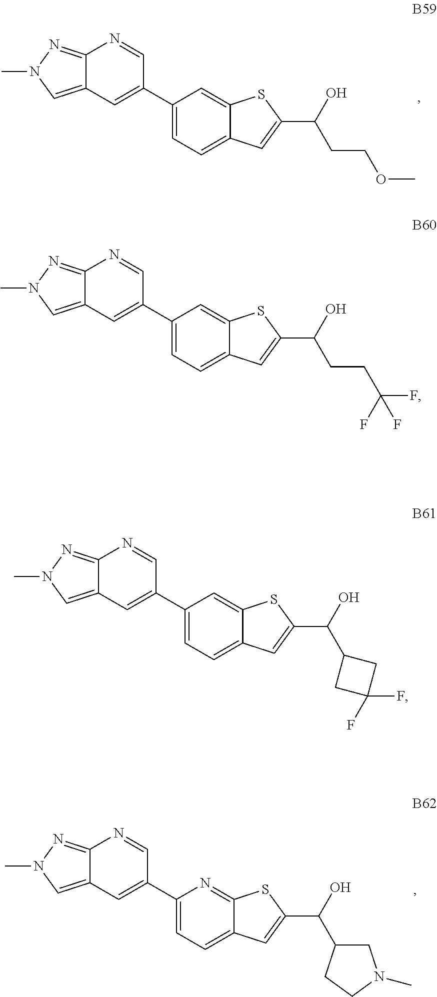

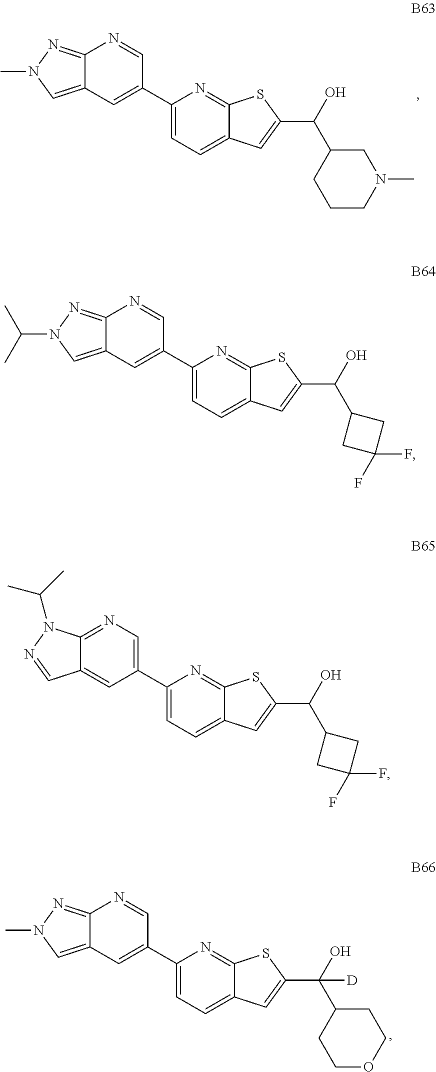

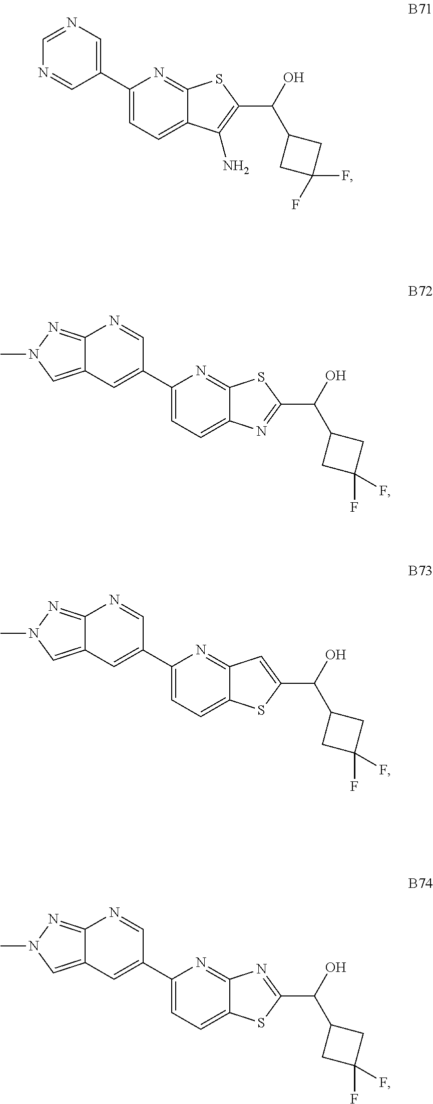 [SMILES: Nc1c(C(O)C2CC(F)(F)C2)sc2nc(-c3cncnc3)ccc12 ; Cn1cc2cc(-c3ccc4nc(C(O)C5CC(F)(F)C5)sc4n3)cnc2n1 ; Cn1cc2cc(-c3ccc4sc(C(O)C5CC(F)(F)C5)cc4n3)cnc2n1 ; Cn1cc2cc(-c3ccc4sc(C(O)C5CC(F)(F)C5)nc4n3)cnc2n1]

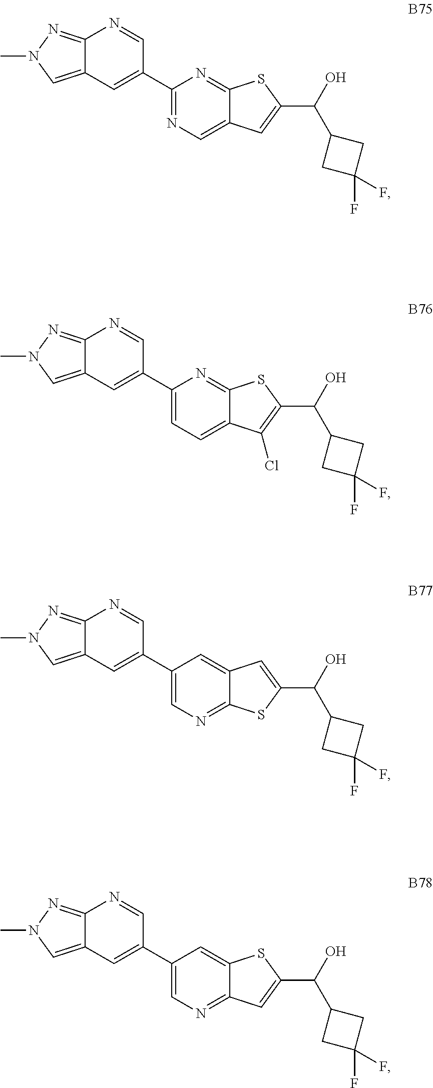 [SMILES: Cn1cc2cc(-c3ncc4cc(C(O)C5CC(F)(F)C5)sc4n3)cnc2n1 ; Cn1cc2cc(-c3ccc4c(Cl)c(C(O)C5CC(F)(F)C5)sc4n3)cnc2n1 ; Cn1cc2cc(-c3cnc4sc(C(O)C5CC(F)(F)C5)cc4c3)cnc2n1 ; Cn1cc2cc(-c3cnc4cc(C(O)C5CC(F)(F)C5)sc4c3)cnc2n1]

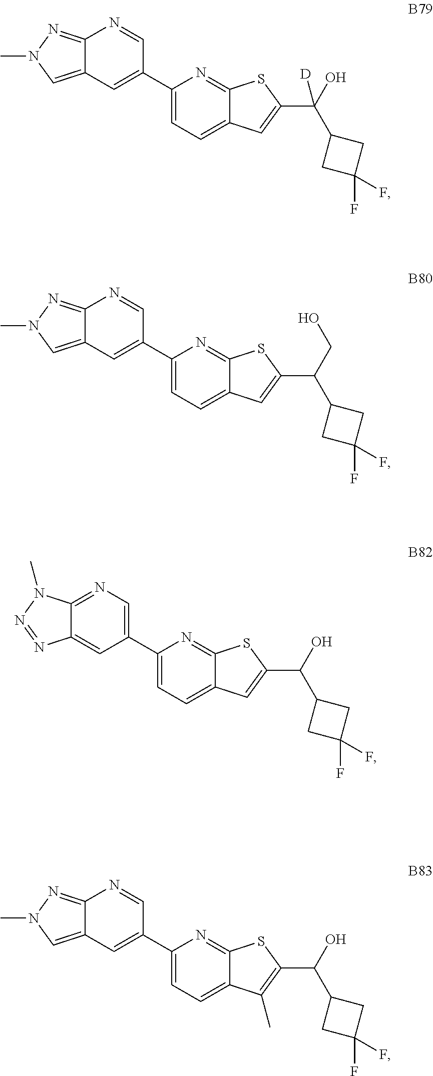 [SMILES: [2H]C(O)(c1cc2ccc(-c3cnc4nn(C)cc4c3)nc2s1)C1CC(F)(F)C1 ; Cn1cc2cc(-c3ccc4cc(C(CO)C5CC(F)(F)C5)sc4n3)cnc2n1 ; Cn1nnc2cc(-c3ccc4cc(C(O)C5CC(F)(F)C5)sc4n3)cnc21 ; Cc1c(C(O)C2CC(F)(F)C2)sc2nc(-c3cnc4nn(C)cc4c3)ccc12]

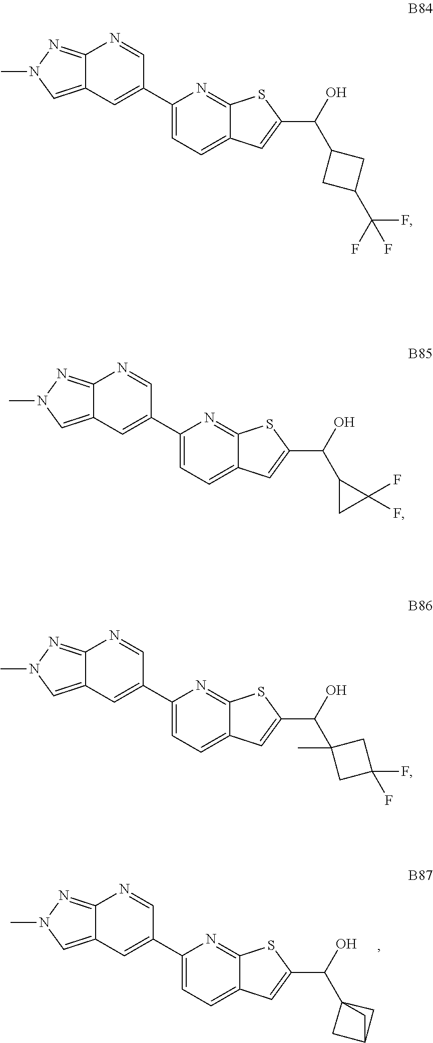 [SMILES: Cn1cc2cc(-c3ccc4cc(C(O)C5CC(C(F)(F)F)C5)sc4n3)cnc2n1 ; Cn1cc2cc(-c3ccc4cc(C(O)C5CC5(F)F)sc4n3)cnc2n1 ; Cn1cc2cc(-c3ccc4cc(C(O)C5(C)CC(F)(F)C5)sc4n3)cnc2n1 ; Cn1cc2cc(-c3ccc4cc(C(O)C56CC(C5)C6)sc4n3)cnc2n1]

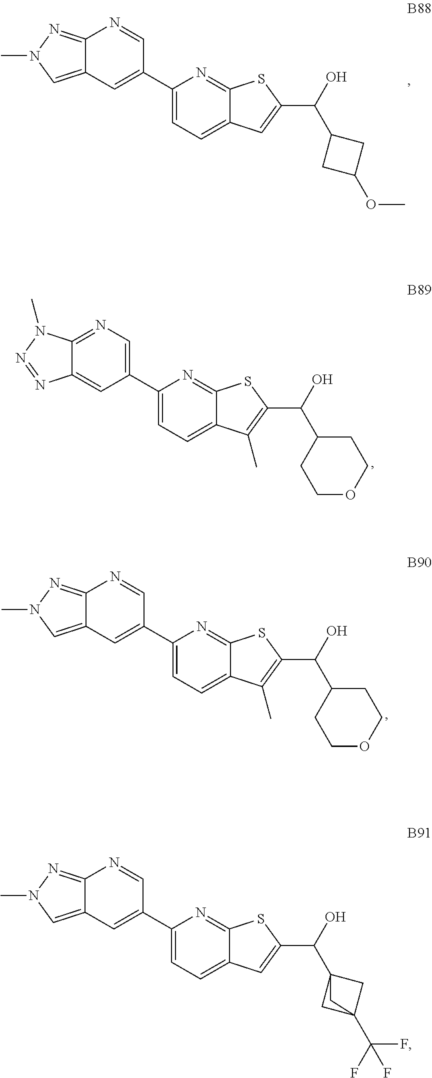

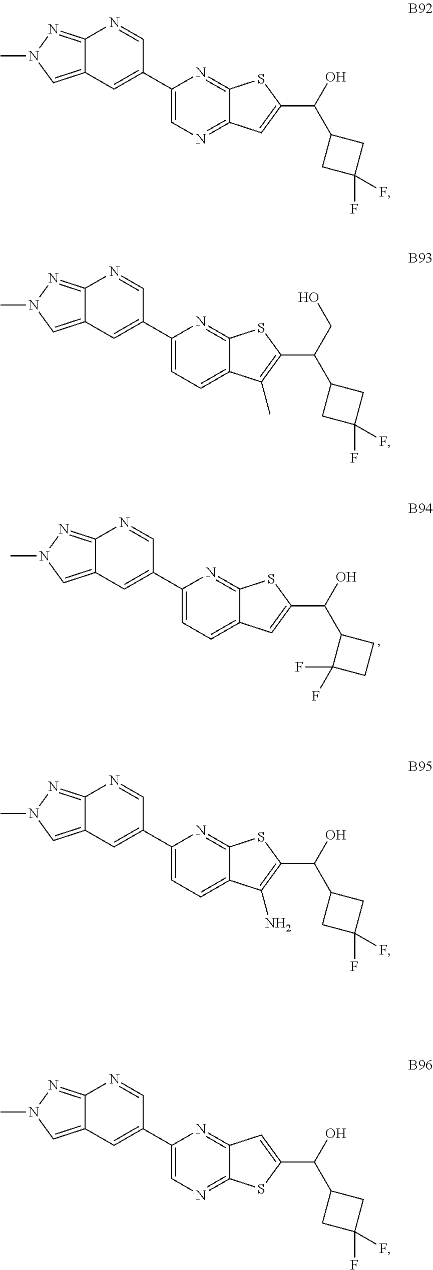 [SMILES: Cn1cc2cc(-c3cnc4cc(C(O)C5CC(F)(F)C5)sc4n3)cnc2n1 ; Cc1c(C(CO)C2CC(F)(F)C2)sc2nc(-c3cnc4nn(C)cc4c3)ccc12 ; Cn1cc2cc(-c3ccc4cc(C(O)C5CCC5(F)F)sc4n3)cnc2n1 ; Cn1cc2cc(-c3ccc4c(N)c(C(O)C5CC(F)(F)C5)sc4n3)cnc2n1 ; Cn1cc2cc(-c3cnc4sc(C(O)C5CC(F)(F)C5)cc4n3)cnc2n1]

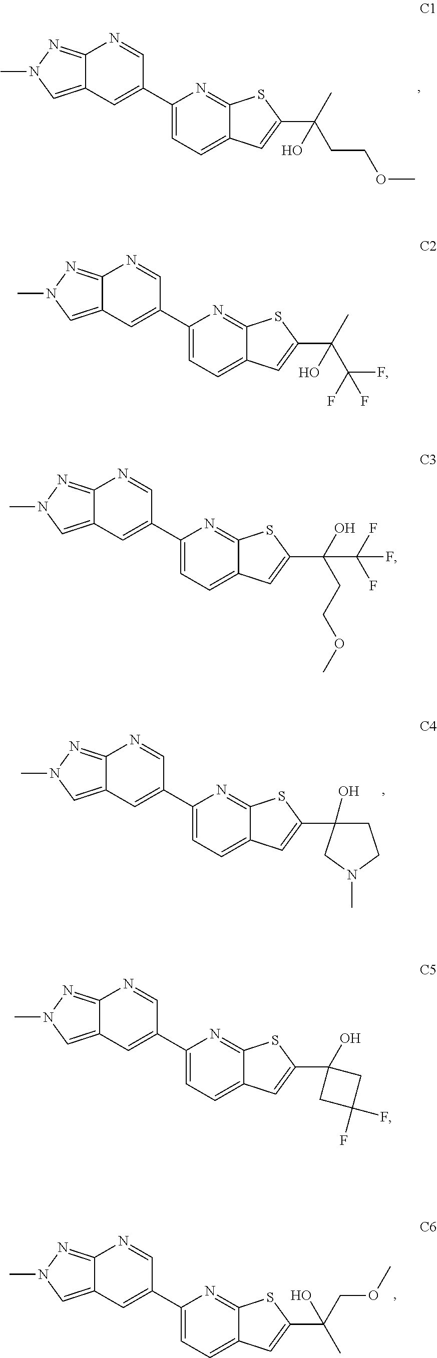

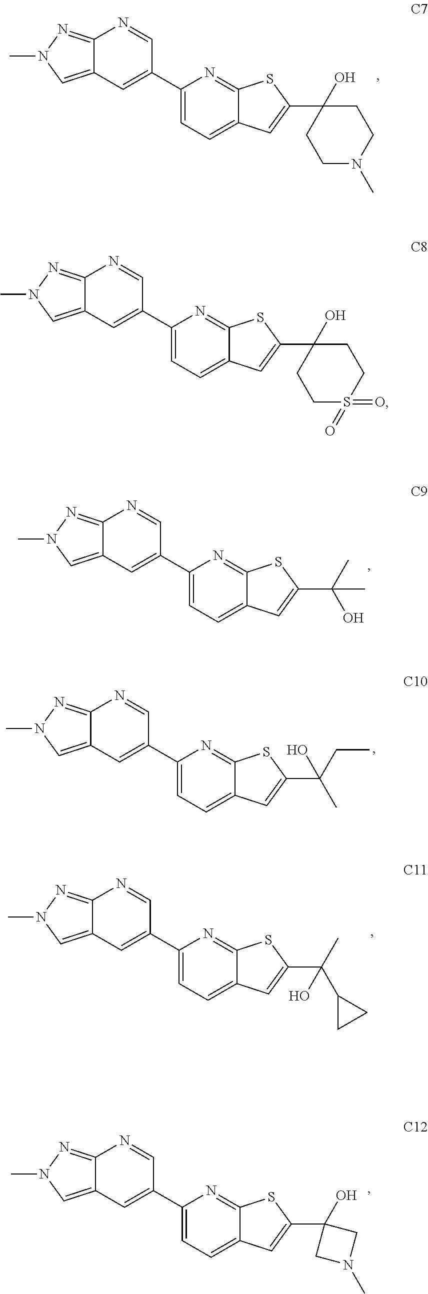

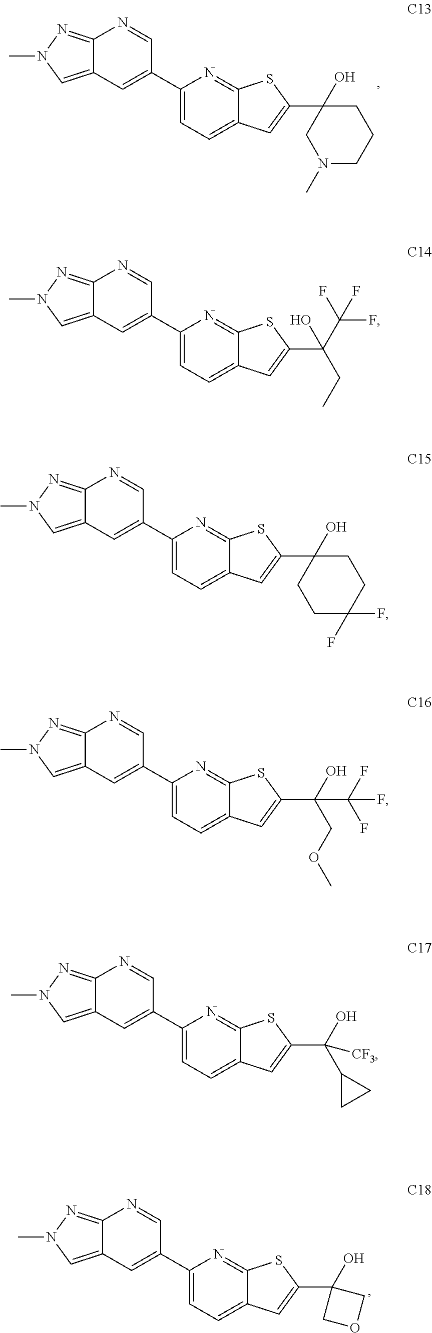 [SMILES: CN1CCCC(O)(c2cc3ccc(-c4cnc5nn(C)cc5c4)nc3s2)C1 ; CCC(O)(c1cc2ccc(-c3cnc4nn(C)cc4c3)nc2s1)C(F)(F)F ; Cn1cc2cc(-c3ccc4cc(C5(O)CCC(F)(F)CC5)sc4n3)cnc2n1 ; COCC(O)(c1cc2ccc(-c3cnc4nn(C)cc4c3)nc2s1)C(F)(F)F ; Cn1cc2cc(-c3ccc4cc(C(O)(C5CC5)C(F)(F)F)sc4n3)cnc2n1 ; Cn1cc2cc(-c3ccc4cc(C5(O)COC5)sc4n3)cnc2n1]

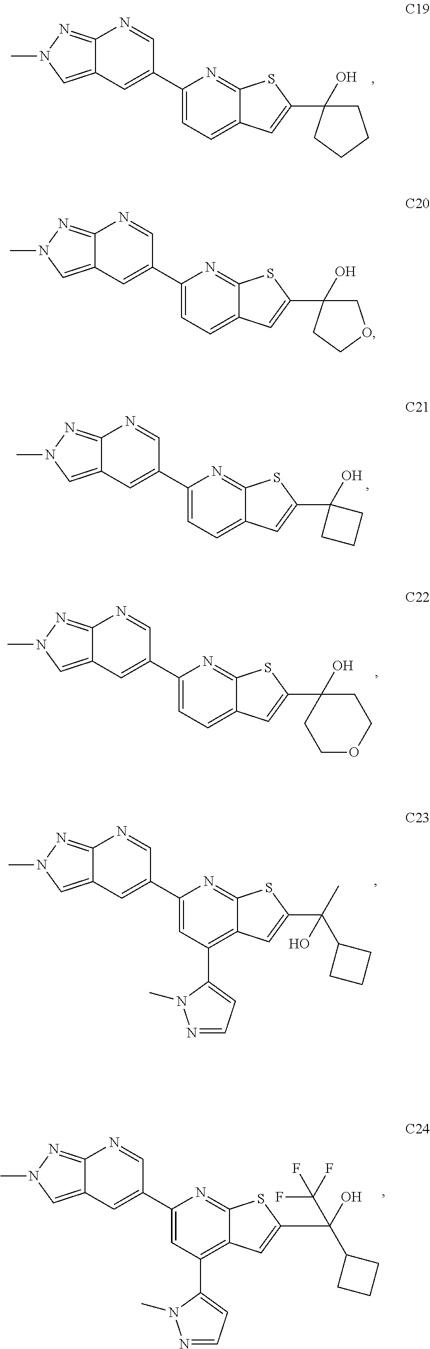 [SMILES: Cn1cc2cc(-c3ccc4cc(C5(O)CCCC5)sc4n3)cnc2n1 ; Cn1cc2cc(-c3ccc4cc(C5(O)CCOC5)sc4n3)cnc2n1 ; Cn1cc2cc(-c3ccc4cc(C5(O)CCC5)sc4n3)cnc2n1 ; Cn1cc2cc(-c3ccc4cc(C5(O)CCOCC5)sc4n3)cnc2n1 ; Cn1cc2cc(-c3cc(-c4ccnn4C)c4cc(C(C)(O)C5CCC5)sc4n3)cnc2n1 ; Cn1cc2cc(-c3cc(-c4ccnn4C)c4cc(C(O)(C5CCC5)C(F)(F)F)sc4n3)cnc2n1]

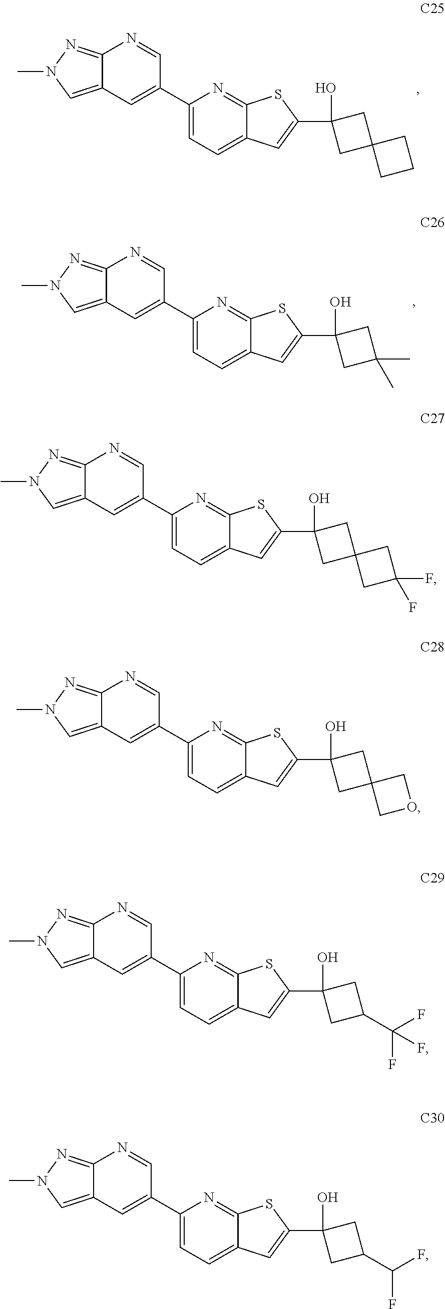

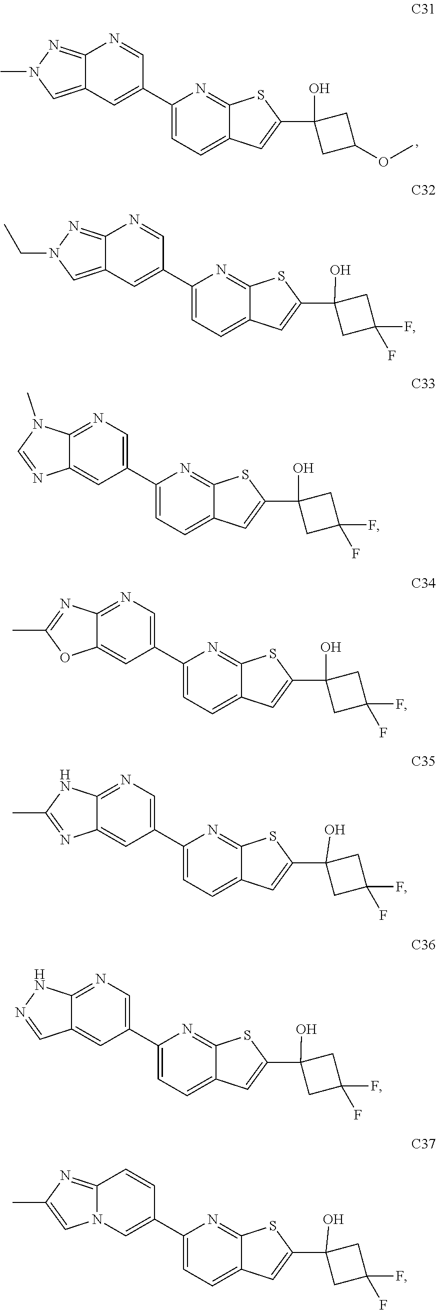

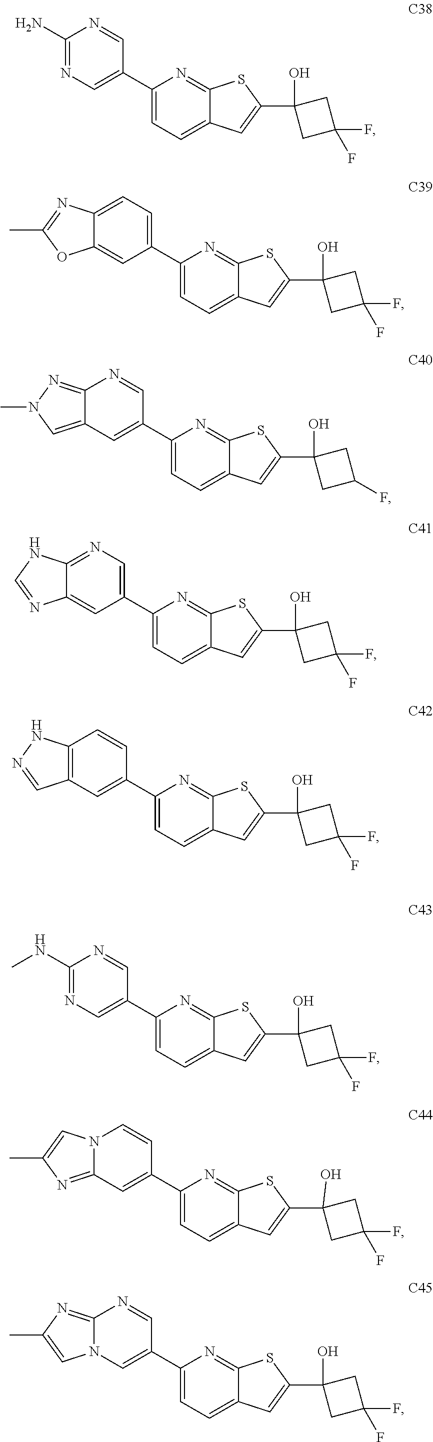

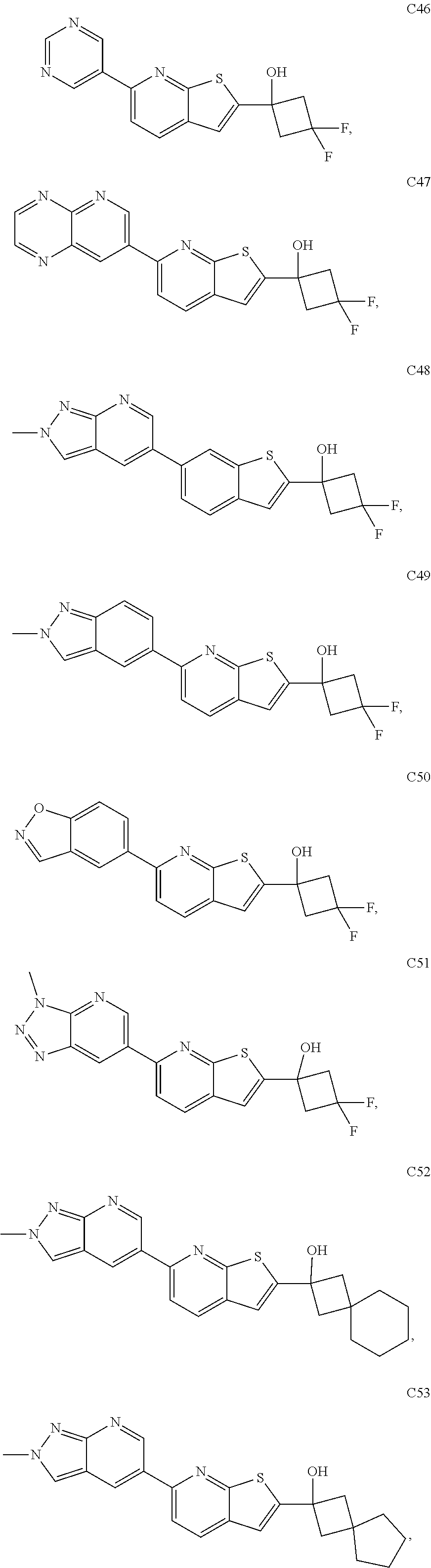

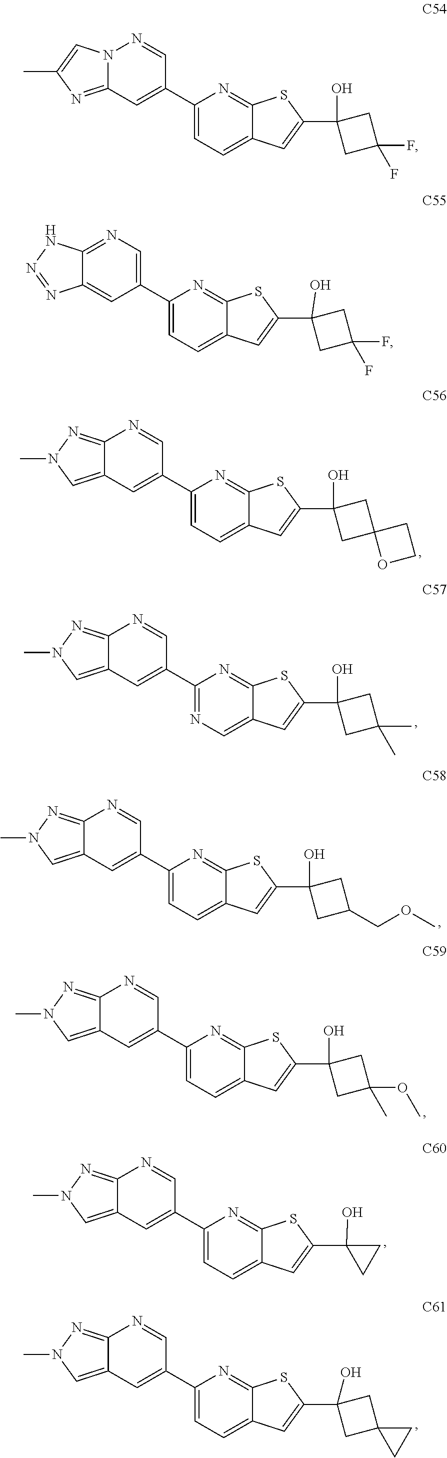

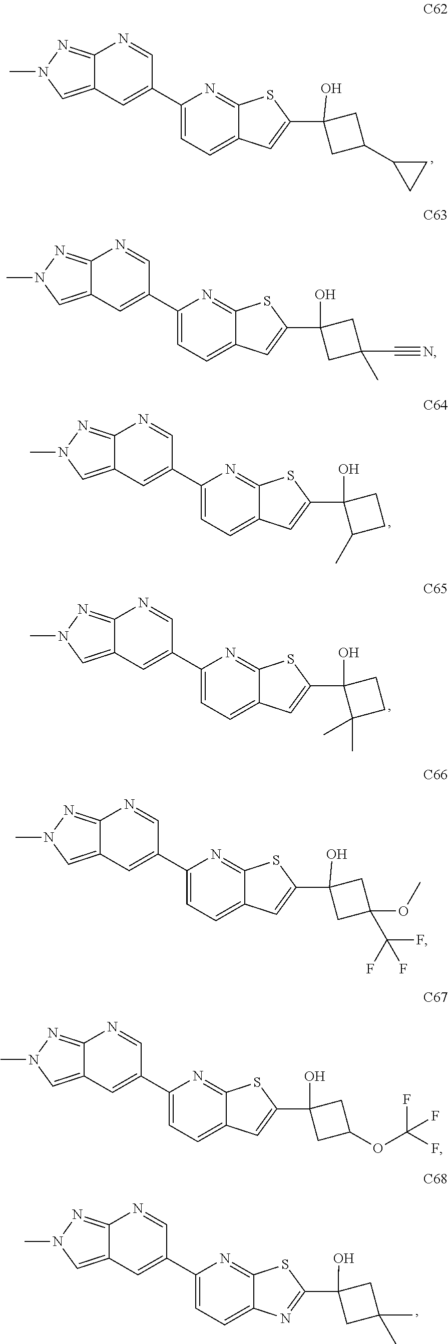

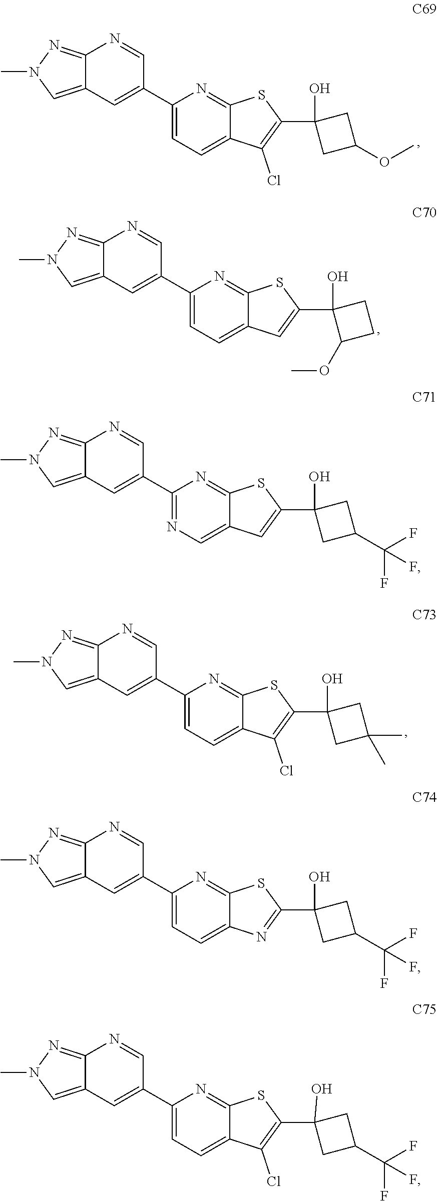

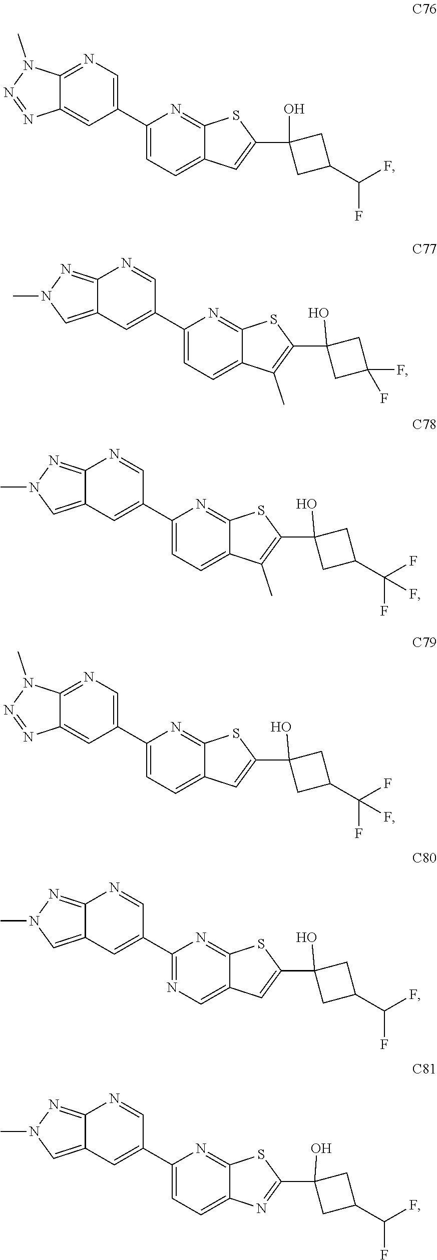 [SMILES: Cn1nnc2cc(-c3ccc4cc(C5(O)CC(C(F)F)C5)sc4n3)cnc21 ; Cc1c(C2(O)CC(F)(F)C2)sc2nc(-c3cnc4nn(C)cc4c3)ccc12 ; Cc1c(C2(O)CC(C(F)(F)F)C2)sc2nc(-c3cnc4nn(C)cc4c3)ccc12 ; Cn1nnc2cc(-c3ccc4cc(C5(O)CC(C(F)(F)F)C5)sc4n3)cnc21 ; Cn1cc2cc(-c3ncc4cc(C5(O)CC(C(F)F)C5)sc4n3)cnc2n1 ; Cn1cc2cc(-c3ccc4nc(C5(O)CC(C(F)F)C5)sc4n3)cnc2n1]

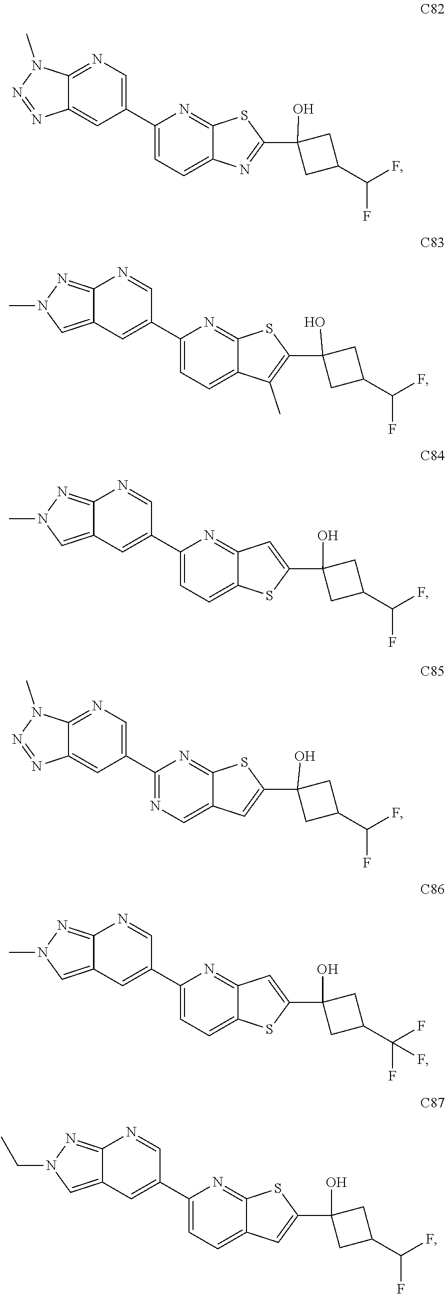 [SMILES: Cn1nnc2cc(-c3ccc4nc(C5(O)CC(C(F)F)C5)sc4n3)cnc21 ; Cc1c(C2(O)CC(C(F)F)C2)sc2nc(-c3cnc4nn(C)cc4c3)ccc12 ; Cn1cc2cc(-c3ccc4sc(C5(O)CC(C(F)F)C5)cc4n3)cnc2n1 ; Cn1nnc2cc(-c3ncc4cc(C5(O)CC(C(F)F)C5)sc4n3)cnc21 ; Cn1cc2cc(-c3ccc4sc(C5(O)CC(C(F)(F)F)C5)cc4n3)cnc2n1 ; CCn1cc2cc(-c3ccc4cc(C5(O)CC(C(F)F)C5)sc4n3)cnc2n1]

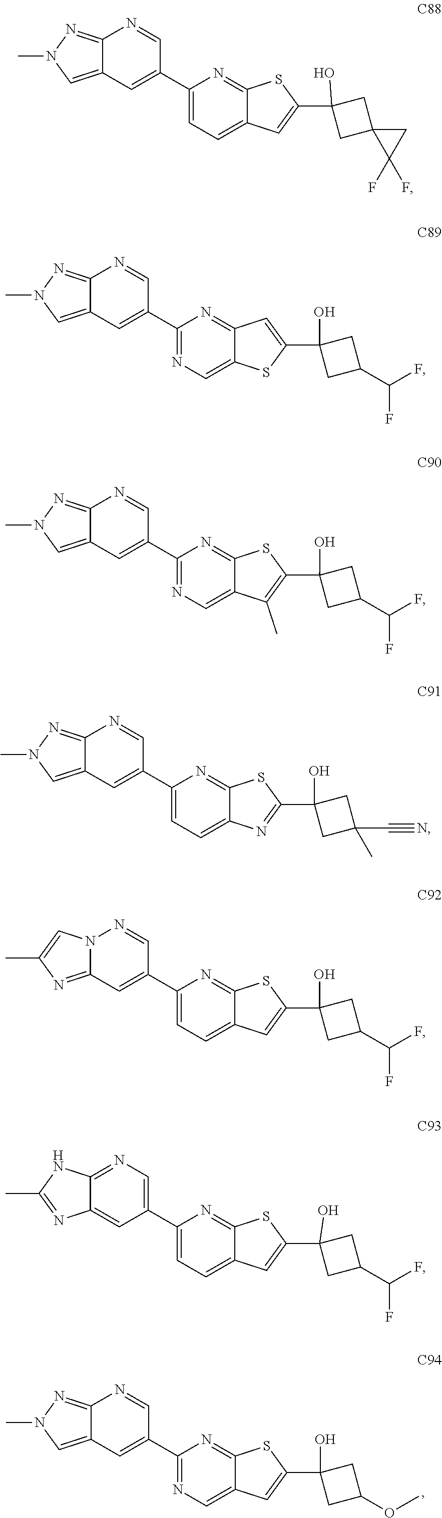

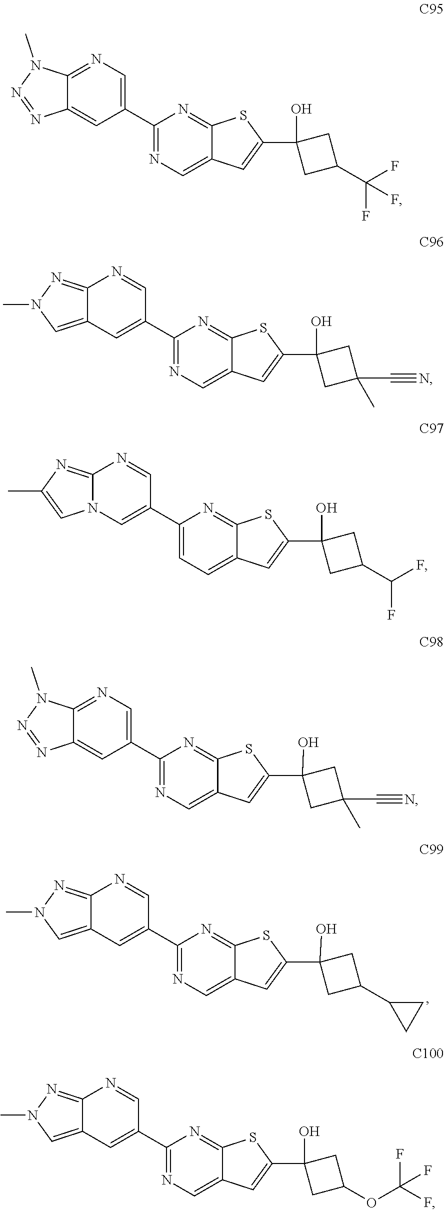 [SMILES: Cn1nnc2cc(-c3ncc4cc(C5(O)CC(C(F)(F)F)C5)sc4n3)cnc21 ; Cn1cc2cc(-c3ncc4cc(C5(O)CC(C)(C#N)C5)sc4n3)cnc2n1 ; Cc1cn2cc(-c3ccc4cc(C5(O)CC(C(F)F)C5)sc4n3)cnc2n1 ; Cn1nnc2cc(-c3ncc4cc(C5(O)CC(C)(C#N)C5)sc4n3)cnc21 ; Cn1cc2cc(-c3ncc4cc(C5(O)CC(C6CC6)C5)sc4n3)cnc2n1 ; Cn1cc2cc(-c3ncc4cc(C5(O)CC(OC(F)(F)F)C5)sc4n3)cnc2n1]

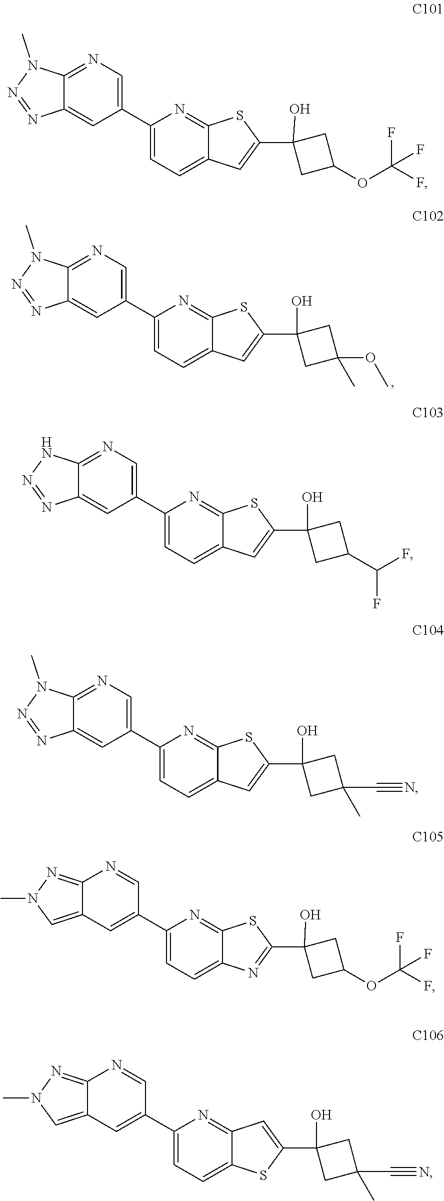

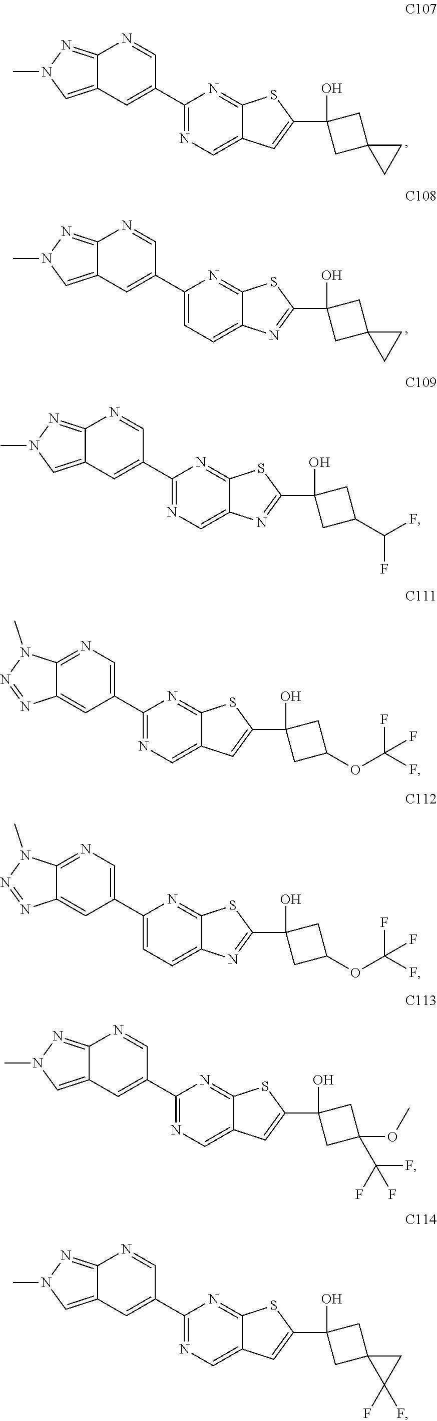

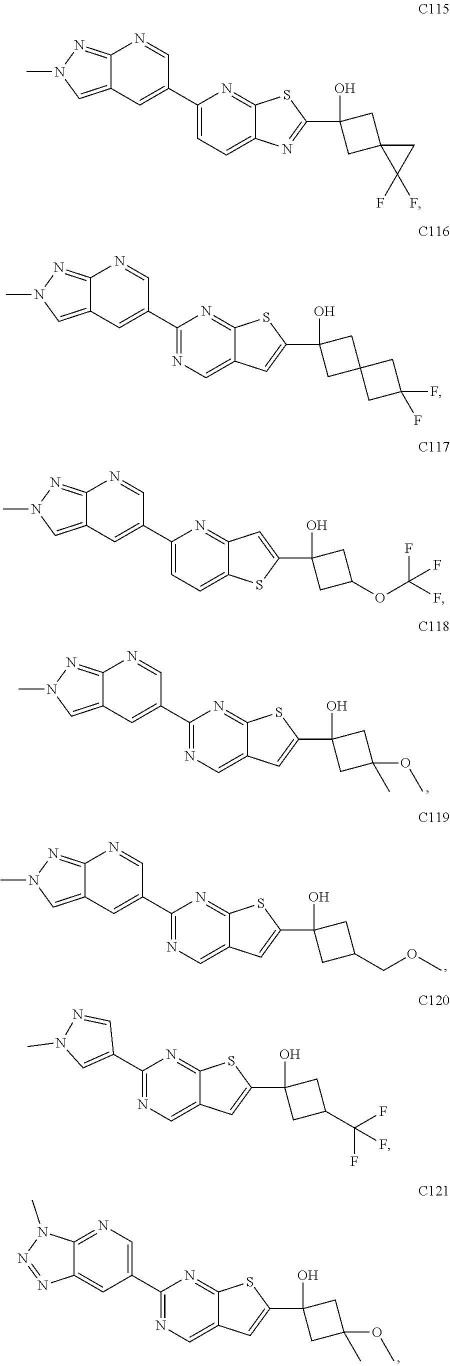

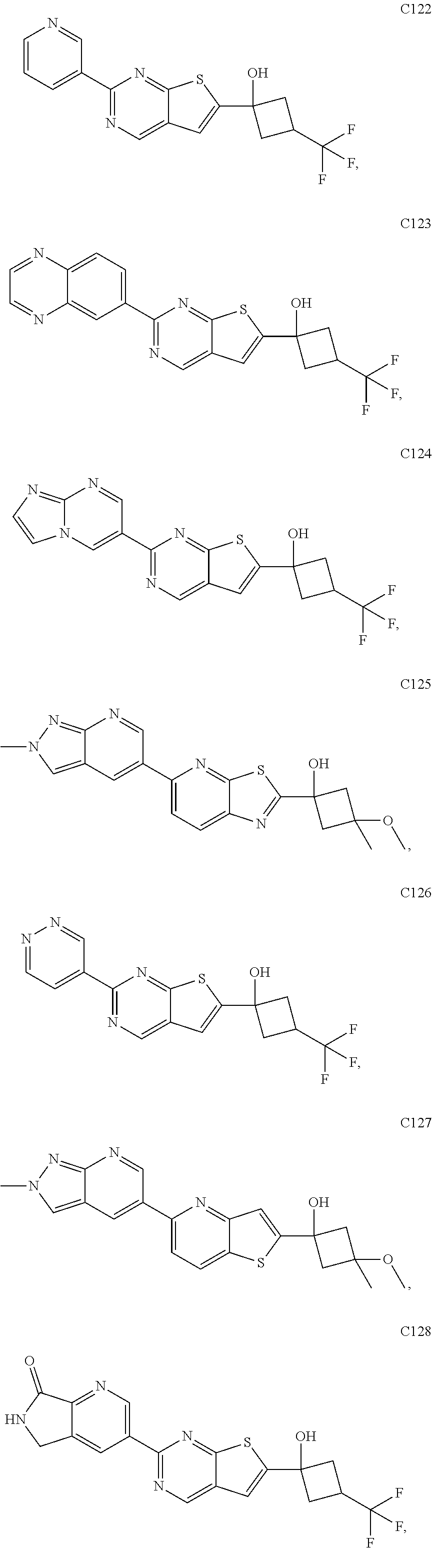

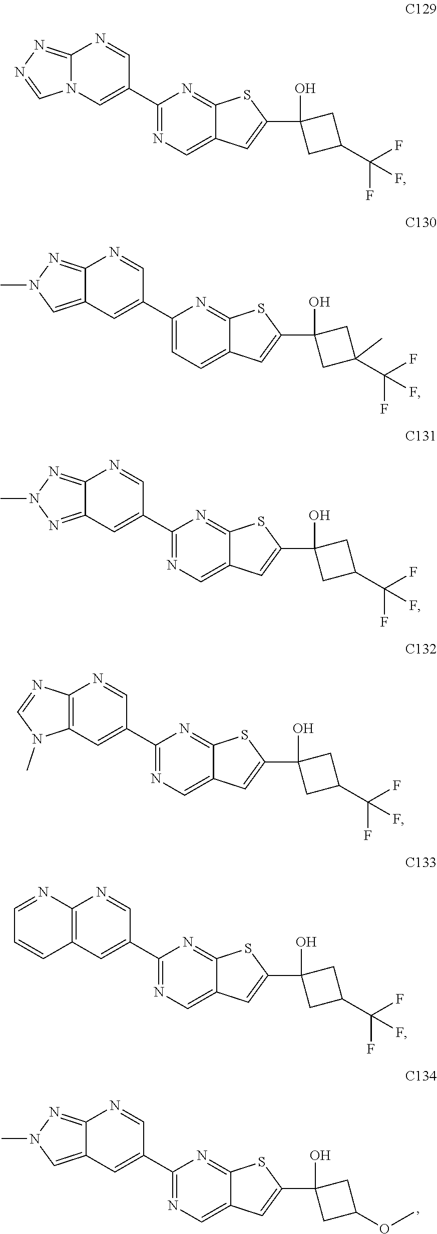

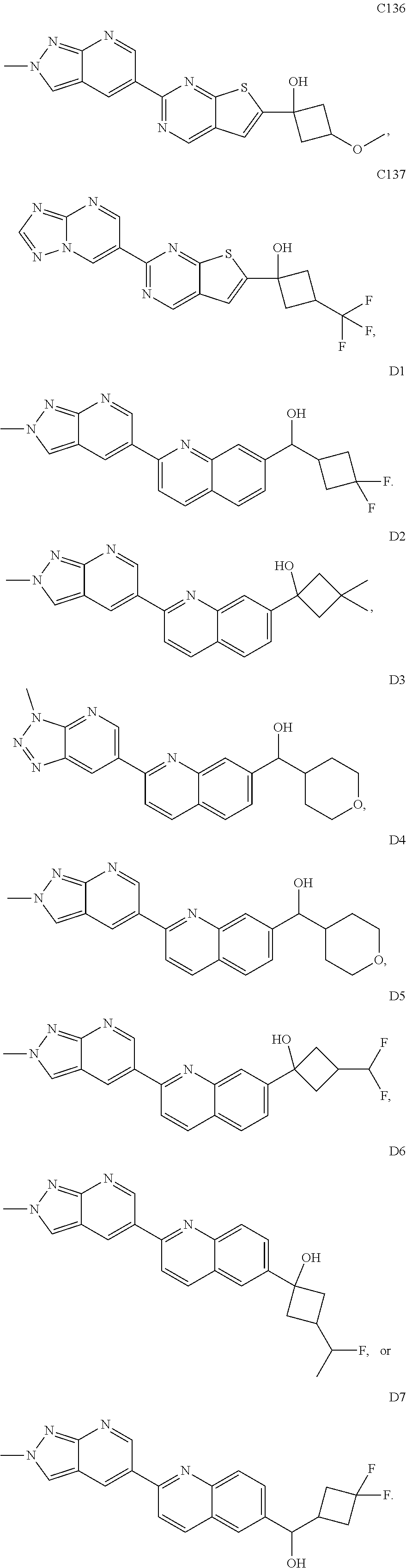

Provided herein as Embodiment 287 is the compound or salt of Embodiment 1, wherein the compound is:

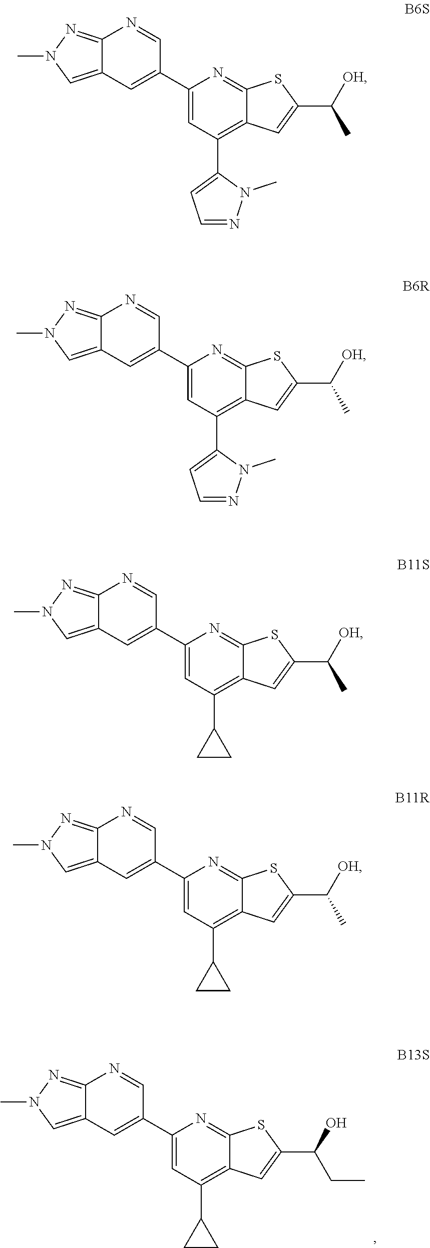

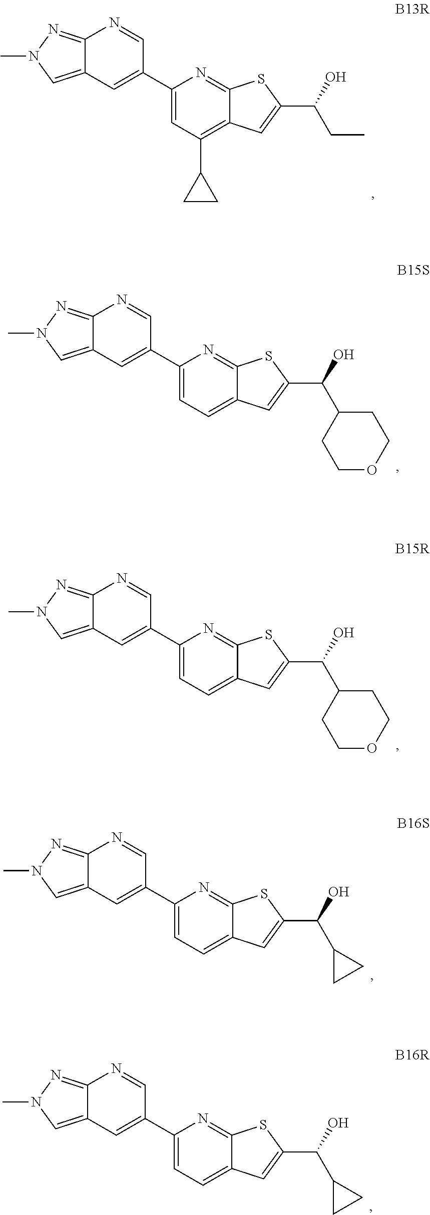

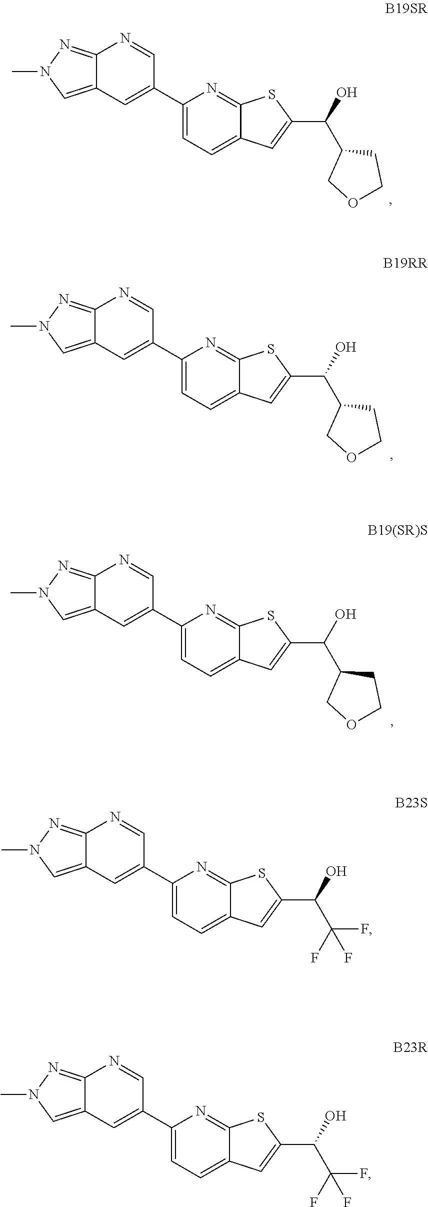 [SMILES: Cn1cc2cc(-c3ccc4cc([C@@H](O)[C@@H]5CCOC5)sc4n3)cnc2n1 ; Cn1cc2cc(-c3ccc4cc([C@H](O)[C@@H]5CCOC5)sc4n3)cnc2n1 ; Cn1cc2cc(-c3ccc4cc(C(O)[C@H]5CCOC5)sc4n3)cnc2n1 ; Cn1cc2cc(-c3ccc4cc([C@@H](O)C(F)(F)F)sc4n3)cnc2n1 ; Cn1cc2cc(-c3ccc4cc([C@H](O)C(F)(F)F)sc4n3)cnc2n1]

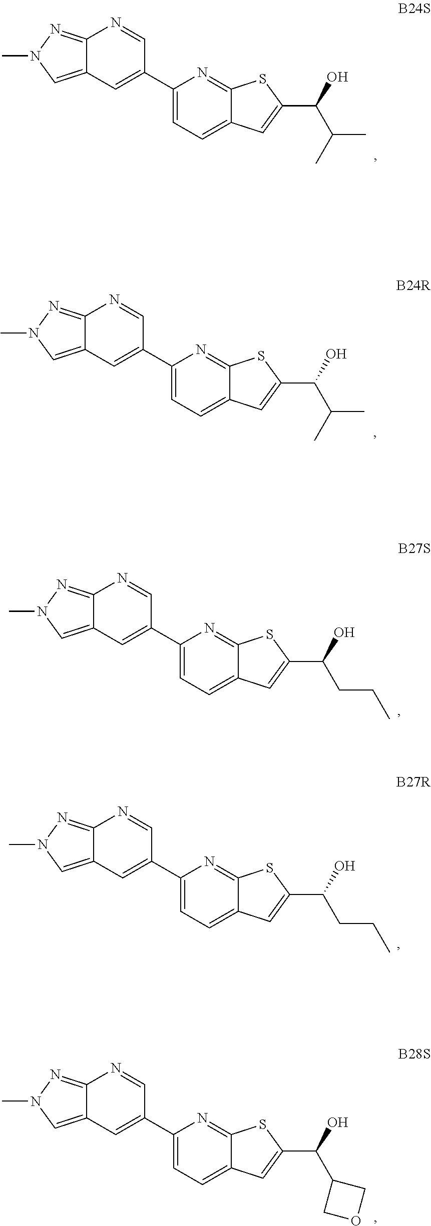

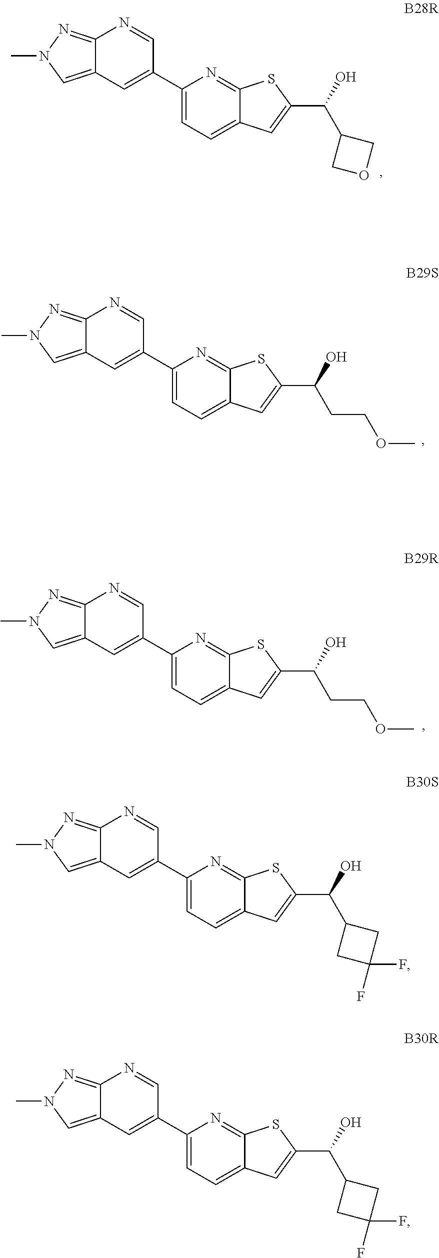

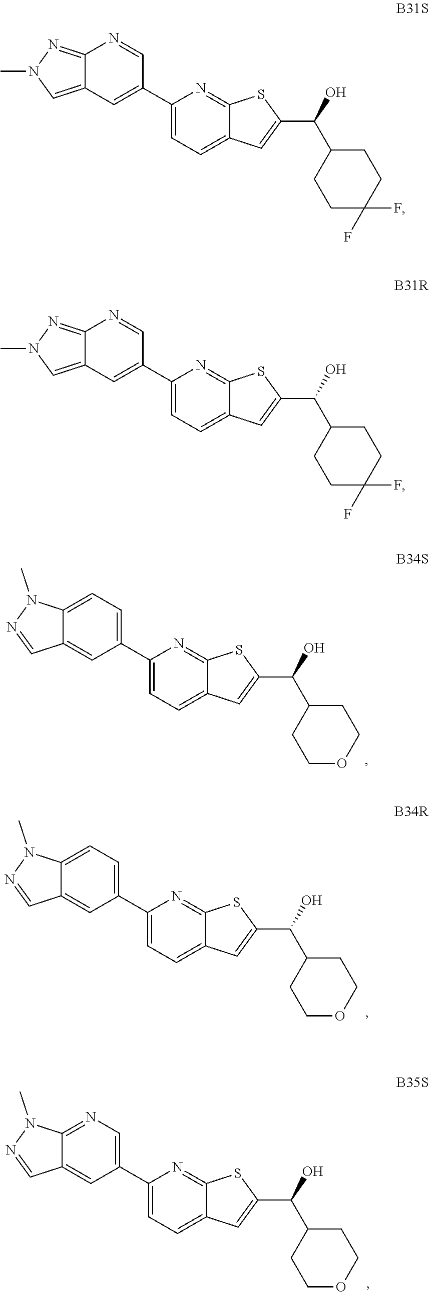 [SMILES: Cn1cc2cc(-c3ccc4cc([C@@H](O)C5CCC(F)(F)CC5)sc4n3)cnc2n1 ; Cn1cc2cc(-c3ccc4cc([C@H](O)C5CCC(F)(F)CC5)sc4n3)cnc2n1 ; Cn1ncc2cc(-c3ccc4cc([C@@H](O)C5CCOCC5)sc4n3)ccc21 ; Cn1ncc2cc(-c3ccc4cc([C@H](O)C5CCOCC5)sc4n3)ccc21 ; Cn1ncc2cc(-c3ccc4cc([C@@H](O)C5CCOCC5)sc4n3)cnc21]

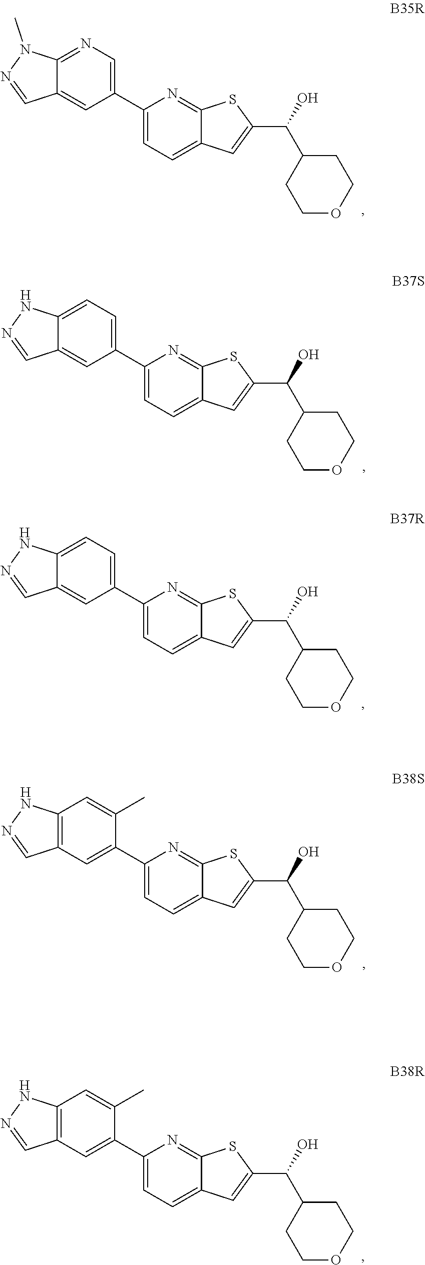 [SMILES: Cn1ncc2cc(-c3ccc4cc([C@H](O)C5CCOCC5)sc4n3)cnc21 ; O[C@H](c1cc2ccc(-c3ccc4[nH]ncc4c3)nc2s1)C1CCOCC1 ; O[C@@H](c1cc2ccc(-c3ccc4[nH]ncc4c3)nc2s1)C1CCOCC1 ; Cc1cc2[nH]ncc2cc1-c1ccc2cc([C@@H](O)C3CCOCC3)sc2n1 ; Cc1cc2[nH]ncc2cc1-c1ccc2cc([C@H](O)C3CCOCC3)sc2n1]

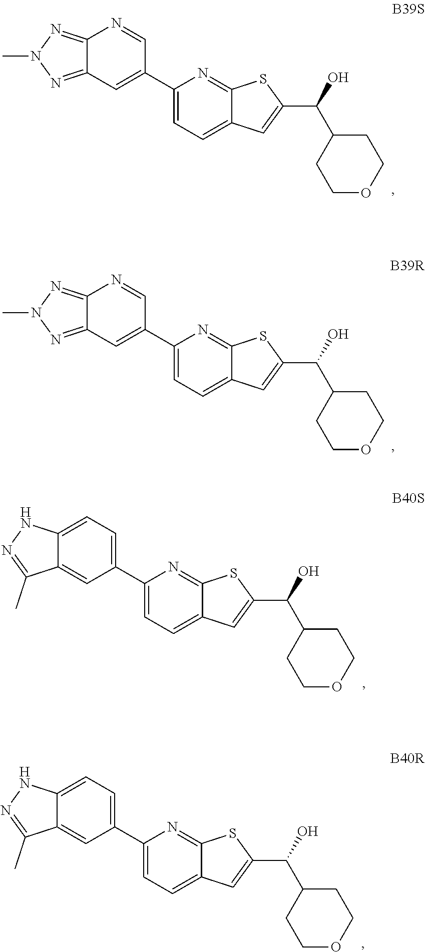 [SMILES: Cn1nc2cc(-c3ccc4cc([C@@H](O)C5CCOCC5)sc4n3)cnc2n1 ; Cn1nc2cc(-c3ccc4cc([C@H](O)C5CCOCC5)sc4n3)cnc2n1 ; Cc1n[nH]c2ccc(-c3ccc4cc([C@@H](O)C5CCOCC5)sc4n3)cc12 ; Cc1n[nH]c2ccc(-c3ccc4cc([C@H](O)C5CCOCC5)sc4n3)cc12]

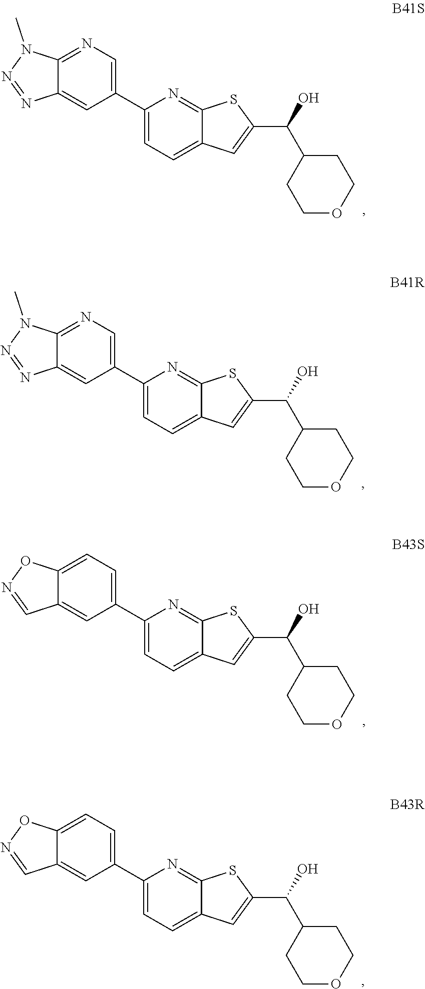 [SMILES: Cn1nnc2cc(-c3ccc4cc([C@@H](O)C5CCOCC5)sc4n3)cnc21 ; Cn1nnc2cc(-c3ccc4cc([C@H](O)C5CCOCC5)sc4n3)cnc21 ; O[C@H](c1cc2ccc(-c3ccc4oncc4c3)nc2s1)C1CCOCC1 ; O[C@@H](c1cc2ccc(-c3ccc4oncc4c3)nc2s1)C1CCOCC1]

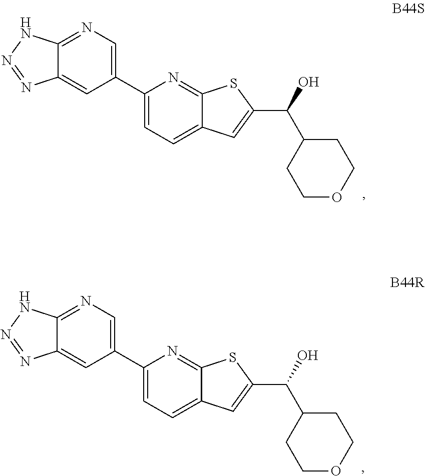 [SMILES: O[C@H](c1cc2ccc(-c3cnc4[nH]nnc4c3)nc2s1)C1CCOCC1 ; O[C@@H](c1cc2ccc(-c3cnc4[nH]nnc4c3)nc2s1)C1CCOCC1]

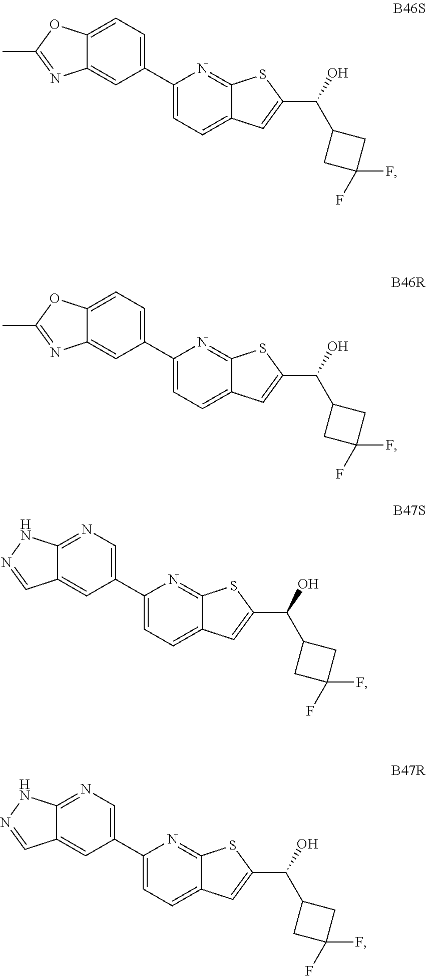 [SMILES: Cc1nc2cc(-c3ccc4cc([C@H](O)C5CC(F)(F)C5)sc4n3)ccc2o1 ; Cc1nc2cc(-c3ccc4cc([C@H](O)C5CC(F)(F)C5)sc4n3)ccc2o1 ; O[C@H](c1cc2ccc(-c3cnc4[nH]ncc4c3)nc2s1)C1CC(F)(F)C1 ; O[C@@H](c1cc2ccc(-c3cnc4[nH]ncc4c3)nc2s1)C1CC(F)(F)C1]

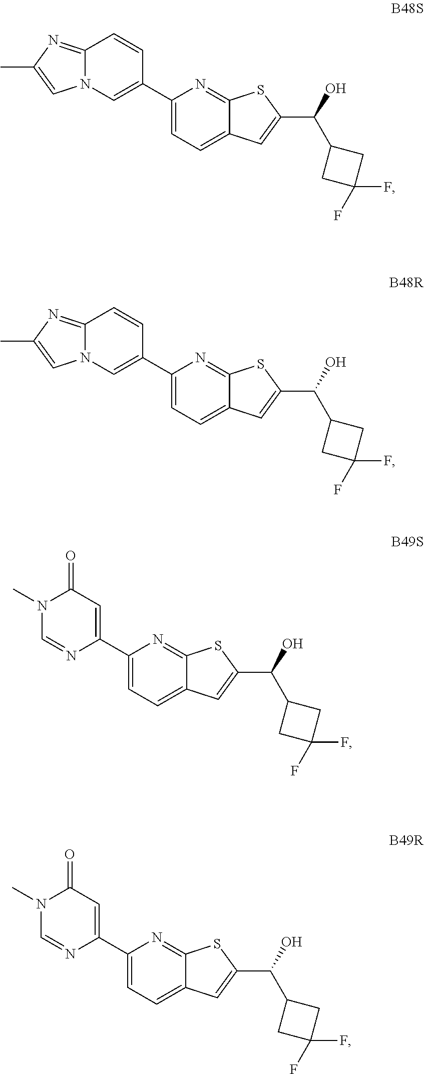 [SMILES: Cc1cn2cc(-c3ccc4cc([C@@H](O)C5CC(F)(F)C5)sc4n3)ccc2n1 ; Cc1cn2cc(-c3ccc4cc([C@H](O)C5CC(F)(F)C5)sc4n3)ccc2n1 ; Cn1cnc(-c2ccc3cc([C@@H](O)C4CC(F)(F)C4)sc3n2)cc1=O ; Cn1cnc(-c2ccc3cc([C@H](O)C4CC(F)(F)C4)sc3n2)cc1=O]

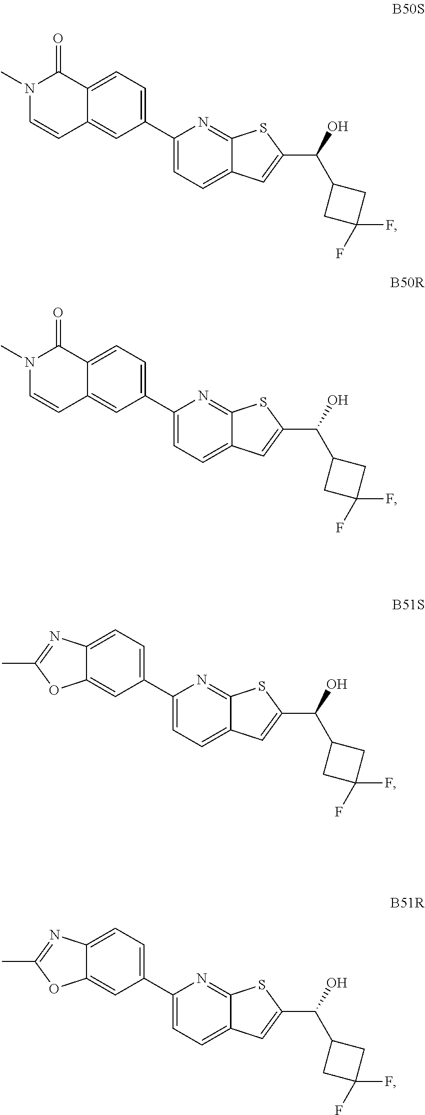 [SMILES: Cn1ccc2cc(-c3ccc4cc([C@@H](O)C5CC(F)(F)C5)sc4n3)ccc2c1=O ; Cn1ccc2cc(-c3ccc4cc([C@H](O)C5CC(F)(F)C5)sc4n3)ccc2c1=O ; Cc1nc2ccc(-c3ccc4cc([C@@H](O)C5CC(F)(F)C5)sc4n3)cc2o1 ; Cc1nc2ccc(-c3ccc4cc([C@H](O)C5CC(F)(F)C5)sc4n3)cc2o1]

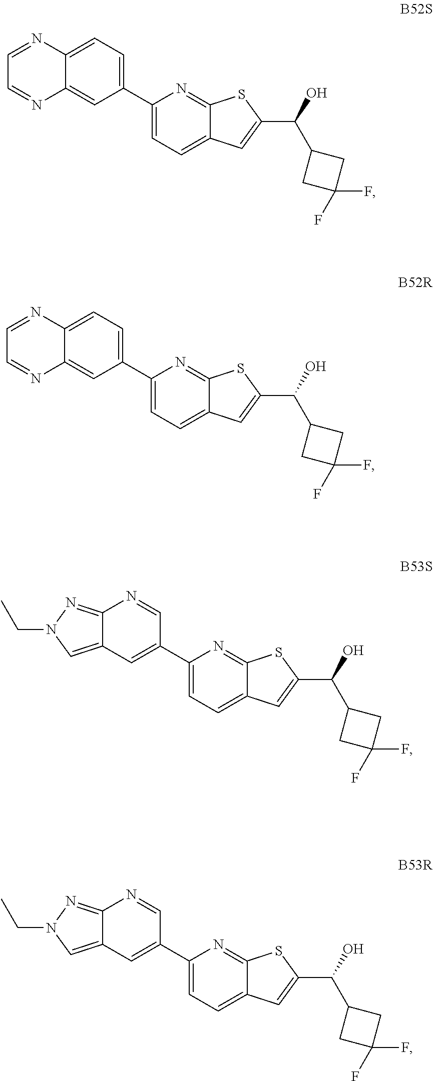 [SMILES: O[C@H](c1cc2ccc(-c3ccc4nccnc4c3)nc2s1)C1CC(F)(F)C1 ; O[C@@H](c1cc2ccc(-c3ccc4nccnc4c3)nc2s1)C1CC(F)(F)C1 ; CCn1cc2cc(-c3ccc4cc([C@@H](O)C5CC(F)(F)C5)sc4n3)cnc2n1 ; CCn1cc2cc(-c3ccc4cc([C@H](O)C5CC(F)(F)C5)sc4n3)cnc2n1]

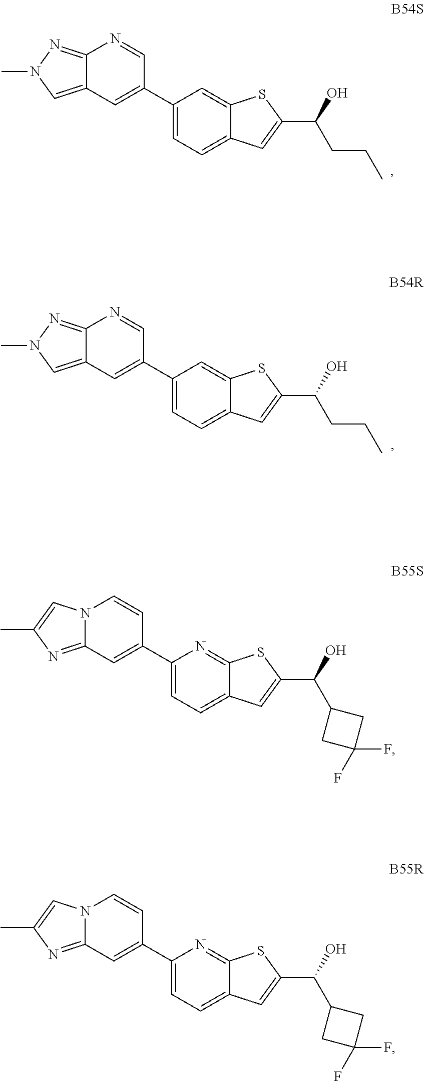

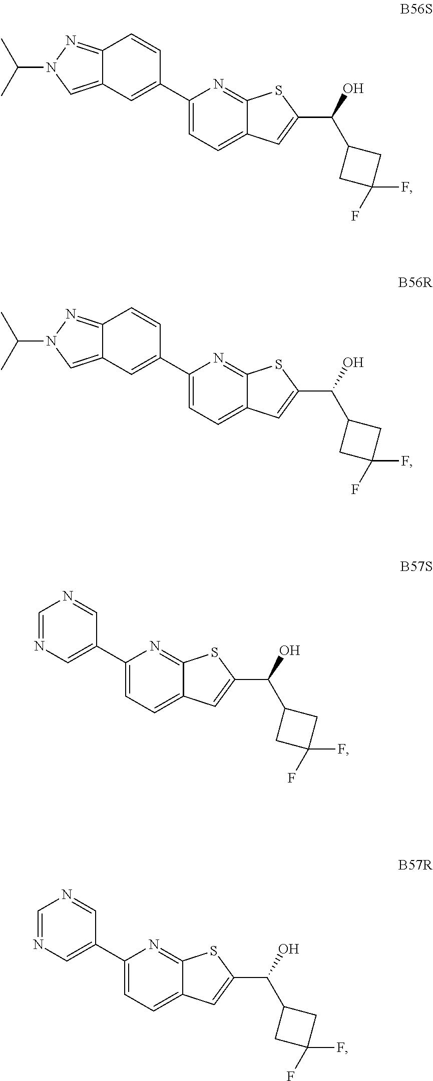 [SMILES: CC(C)n1cc2cc(-c3ccc4cc([C@@H](O)C5CC(F)(F)C5)sc4n3)ccc2n1 ; CC(C)n1cc2cc(-c3ccc4cc([C@H](O)C5CC(F)(F)C5)sc4n3)ccc2n1 ; O[C@H](c1cc2ccc(-c3cncnc3)nc2s1)C1CC(F)(F)C1 ; O[C@@H](c1cc2ccc(-c3cncnc3)nc2s1)C1CC(F)(F)C1]

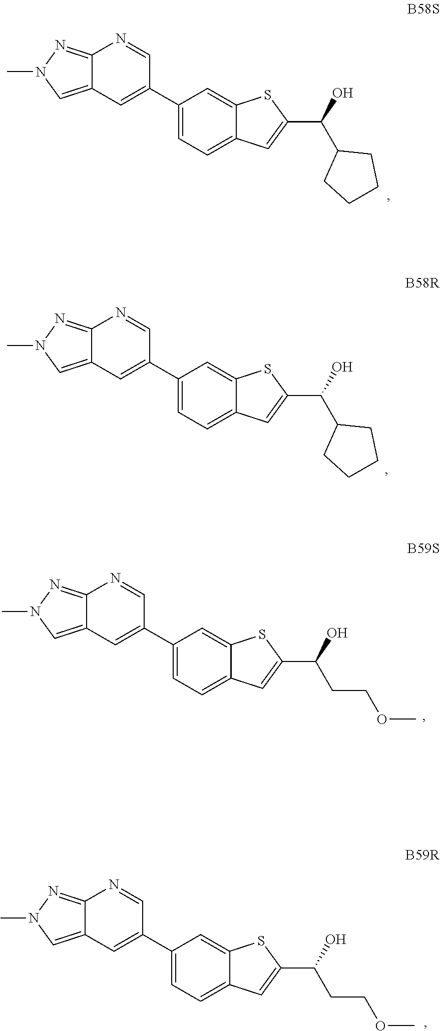

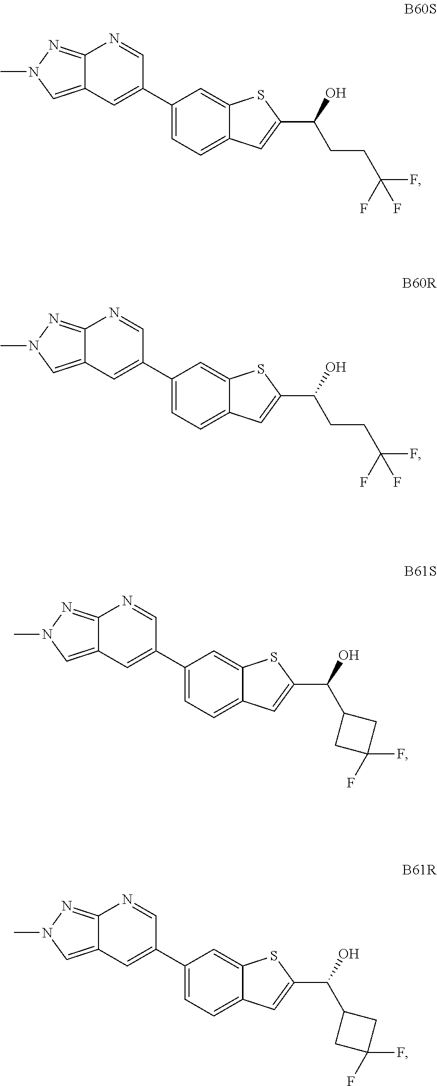 [SMILES: Cn1cc2cc(-c3ccc4cc([C@@H](O)CCC(F)(F)F)sc4c3)cnc2n1 ; Cn1cc2cc(-c3ccc4cc([C@H](O)CCC(F)(F)F)sc4c3)cnc2n1 ; Cn1cc2cc(-c3ccc4cc([C@@H](O)C5CC(F)(F)C5)sc4c3)cnc2n1 ; Cn1cc2cc(-c3ccc4cc([C@H](O)C5CC(F)(F)C5)sc4c3)cnc2n1]

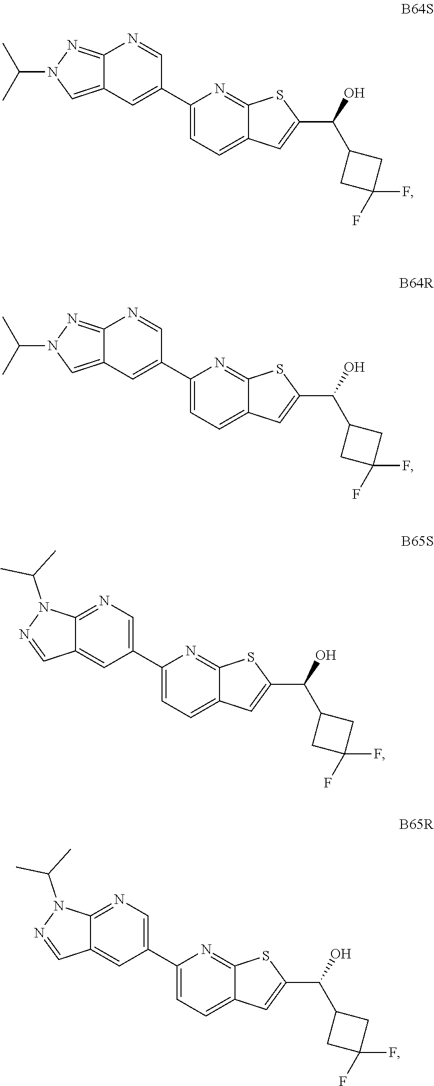 [SMILES: CC(C)n1cc2cc(-c3ccc4cc([C@@H](O)C5CC(F)(F)C5)sc4n3)cnc2n1 ; CC(C)n1cc2cc(-c3ccc4cc([C@H](O)C5CC(F)(F)C5)sc4n3)cnc2n1 ; CC(C)n1ncc2cc(-c3ccc4cc([C@@H](O)C5CC(F)(F)C5)sc4n3)cnc21 ; CC(C)n1ncc2cc(-c3ccc4cc([C@H](O)C5CC(F)(F)C5)sc4n3)cnc21]

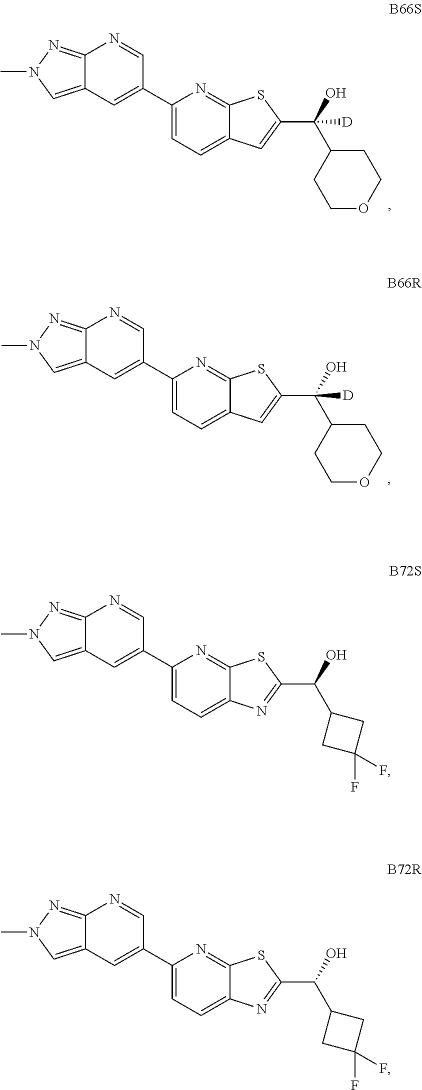 [SMILES: [2H][C@@](O)(c1cc2ccc(-c3cnc4nn(C)cc4c3)nc2s1)C1CCOCC1 ; [2H][C@](O)(c1cc2ccc(-c3cnc4nn(C)cc4c3)nc2s1)C1CCOCC1 ; Cn1cc2cc(-c3ccc4nc([C@@H](O)C5CC(F)(F)C5)sc4n3)cnc2n1 ; Cn1cc2cc(-c3ccc4nc([C@H](O)C5CC(F)(F)C5)sc4n3)cnc2n1]

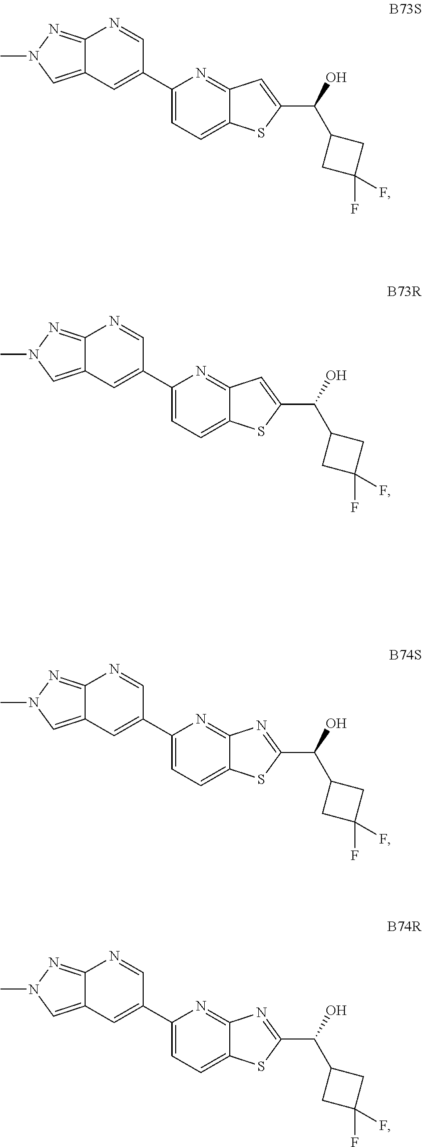 [SMILES: Cn1cc2cc(-c3ccc4sc([C@@H](O)C5CC(F)(F)C5)cc4n3)cnc2n1 ; Cn1cc2cc(-c3ccc4sc([C@H](O)C5CC(F)(F)C5)cc4n3)cnc2n1 ; Cn1cc2cc(-c3ccc4sc([C@@H](O)C5CC(F)(F)C5)nc4n3)cnc2n1 ; Cn1cc2cc(-c3ccc4sc([C@H](O)C5CC(F)(F)C5)nc4n3)cnc2n1]

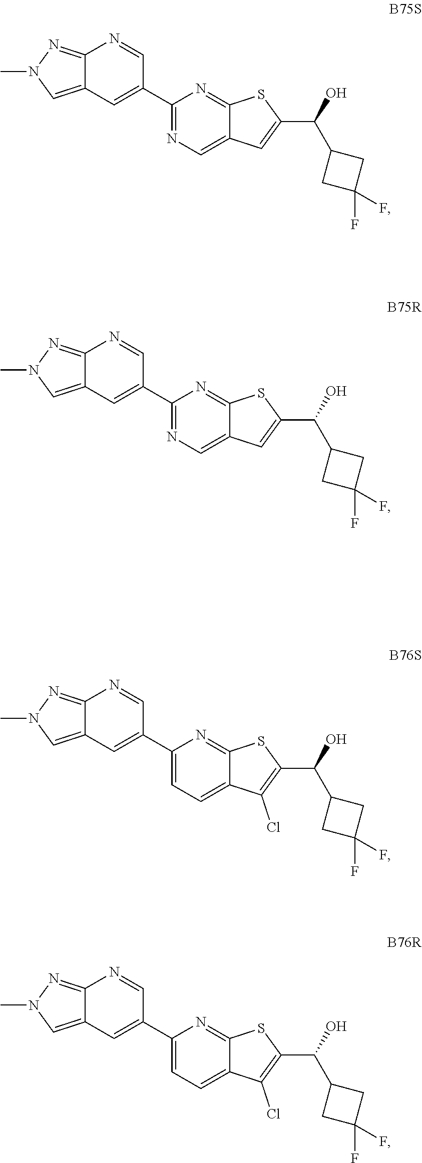 [SMILES: Cn1cc2cc(-c3ncc4cc([C@@H](O)C5CC(F)(F)C5)sc4n3)cnc2n1 ; Cn1cc2cc(-c3ncc4cc([C@H](O)C5CC(F)(F)C5)sc4n3)cnc2n1 ; Cn1cc2cc(-c3ccc4c(Cl)c([C@@H](O)C5CC(F)(F)C5)sc4n3)cnc2n1 ; Cn1cc2cc(-c3ccc4c(Cl)c([C@H](O)C5CC(F)(F)C5)sc4n3)cnc2n1]

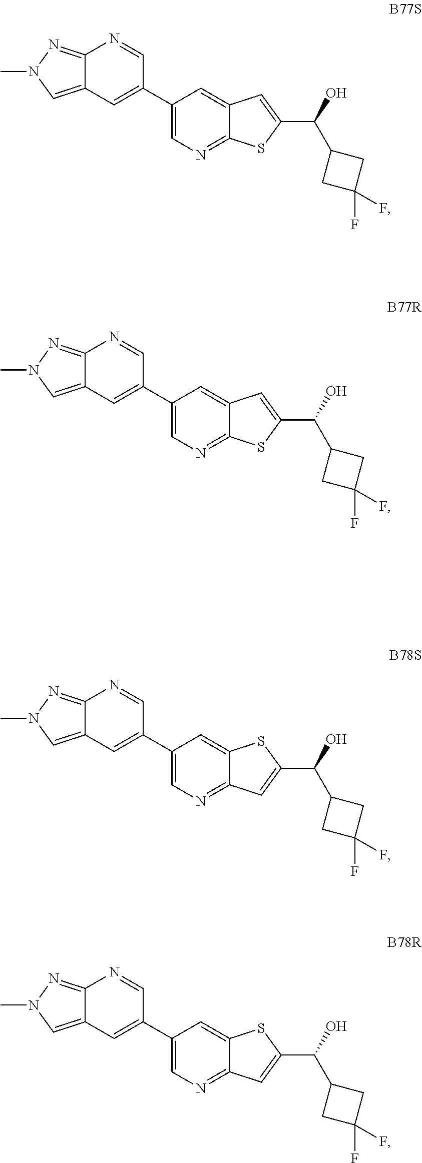 [SMILES: Cn1cc2cc(-c3cnc4sc([C@@H](O)C5CC(F)(F)C5)cc4c3)cnc2n1 ; Cn1cc2cc(-c3cnc4sc([C@H](O)C5CC(F)(F)C5)cc4c3)cnc2n1 ; Cn1cc2cc(-c3cnc4cc([C@@H](O)C5CC(F)(F)C5)sc4c3)cnc2n1 ; Cn1cc2cc(-c3cnc4cc([C@H](O)C5CC(F)(F)C5)sc4c3)cnc2n1]

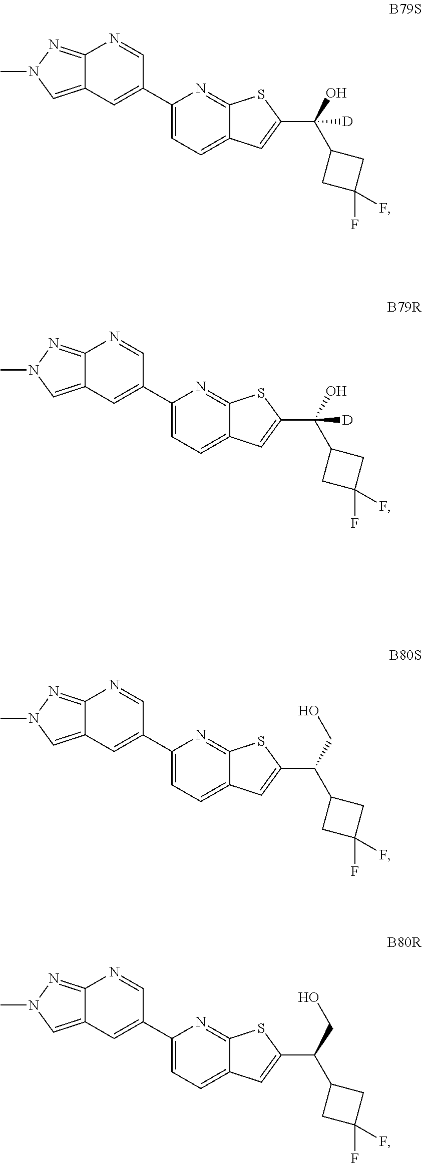 [SMILES: [2H][C@@](O)(c1cc2ccc(-c3cnc4nn(C)cc4c3)nc2s1)C1CC(F)(F)C1 ; [2H][C@](O)(c1cc2ccc(-c3cnc4nn(C)cc4c3)nc2s1)C1CC(F)(F)C1 ; Cn1cc2cc(-c3ccc4cc([C@H](CO)C5CC(F)(F)C5)sc4n3)cnc2n1 ; Cn1cc2cc(-c3ccc4cc([C@@H](CO)C5CC(F)(F)C5)sc4n3)cnc2n1]

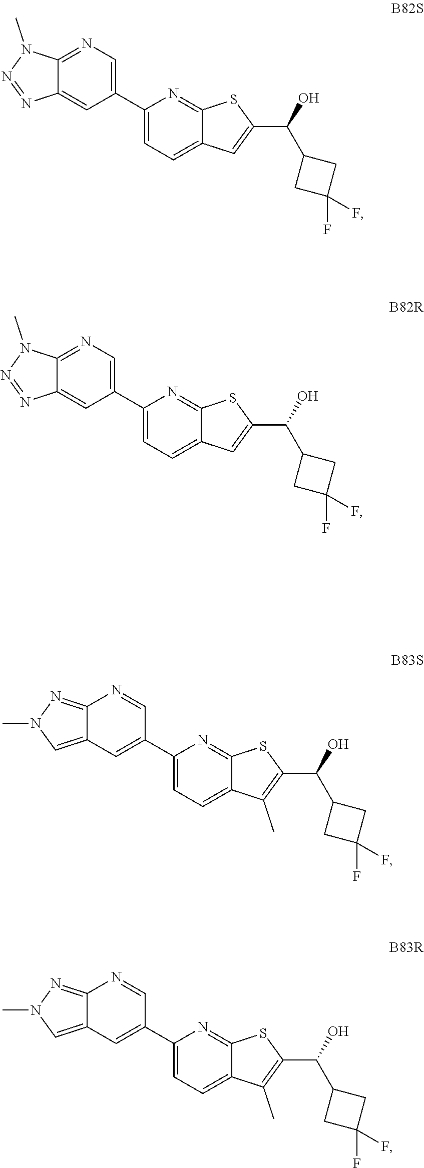 [SMILES: Cn1nnc2cc(-c3ccc4cc([C@@H](O)C5CC(F)(F)C5)sc4n3)cnc21 ; Cn1nnc2cc(-c3ccc4cc([C@H](O)C5CC(F)(F)C5)sc4n3)cnc21 ; Cc1c([C@@H](O)C2CC(F)(F)C2)sc2nc(-c3cnc4nn(C)cc4c3)ccc12 ; Cc1c([C@H](O)C2CC(F)(F)C2)sc2nc(-c3cnc4nn(C)cc4c3)ccc12]

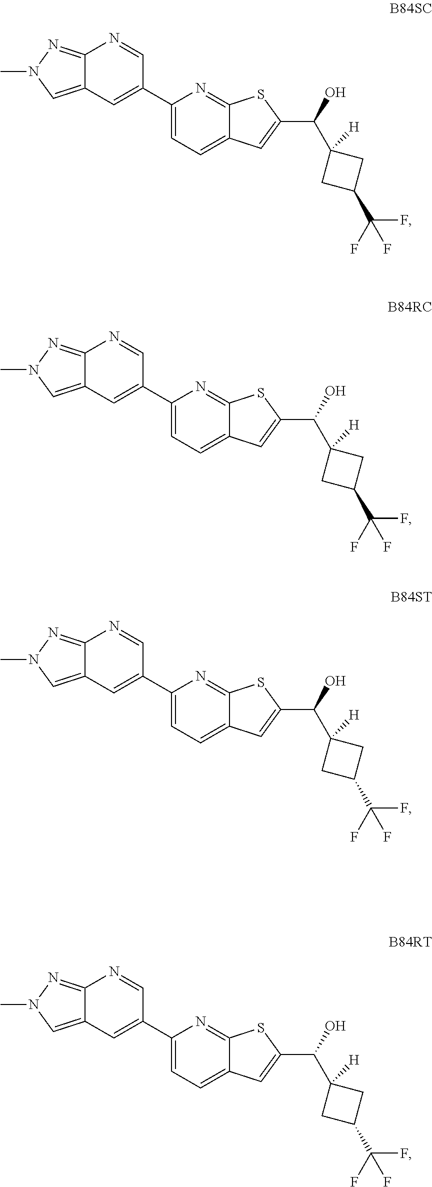 [SMILES: Cn1cc2cc(-c3ccc4cc([C@@H](O)[C@H]5C[C@@H](C(F)(F)F)C5)sc4n3)cnc2n1 ; Cn1cc2cc(-c3ccc4cc([C@H](O)[C@H]5C[C@@H](C(F)(F)F)C5)sc4n3)cnc2n1 ; Cn1cc2cc(-c3ccc4cc([C@@H](O)[C@H]5C[C@H](C(F)(F)F)C5)sc4n3)cnc2n1 ; Cn1cc2cc(-c3ccc4cc([C@H](O)[C@H]5C[C@H](C(F)(F)F)C5)sc4n3)cnc2n1]

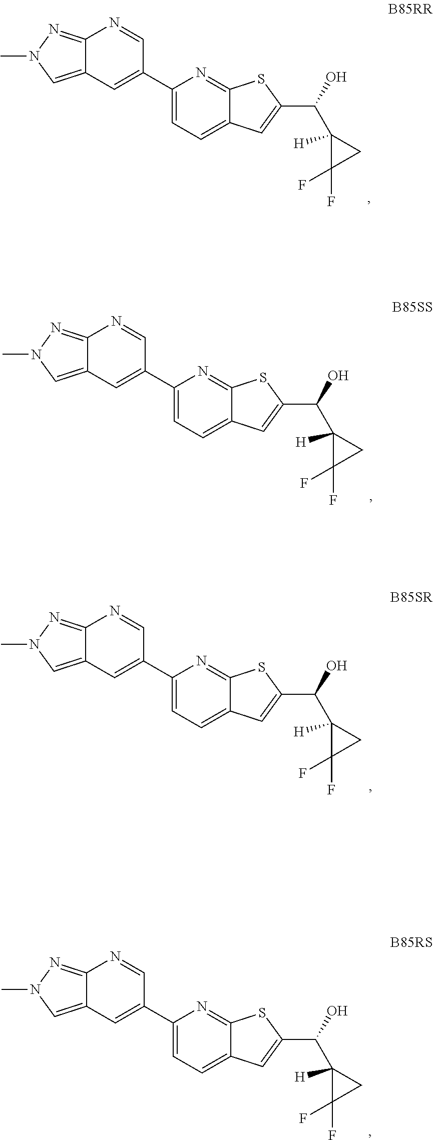 [SMILES: Cn1cc2cc(-c3ccc4cc([C@H](O)[C@H]5CC5(F)F)sc4n3)cnc2n1 ; Cn1cc2cc(-c3ccc4cc([C@@H](O)[C@@H]5CC5(F)F)sc4n3)cnc2n1 ; Cn1cc2cc(-c3ccc4cc([C@@H](O)[C@H]5CC5(F)F)sc4n3)cnc2n1 ; Cn1cc2cc(-c3ccc4cc([C@H](O)[C@@H]5CC5(F)F)sc4n3)cnc2n1]

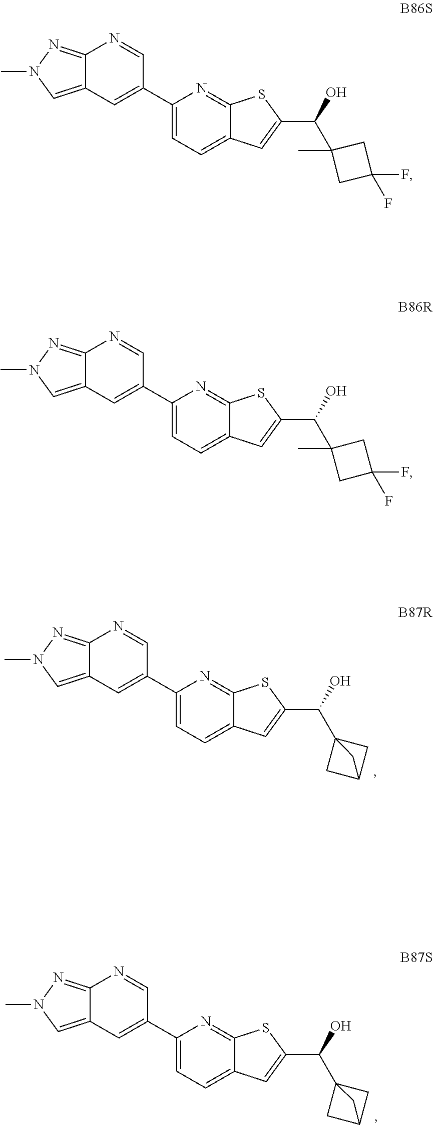 [SMILES: Cn1cc2cc(-c3ccc4cc([C@@H](O)C5(C)CC(F)(F)C5)sc4n3)cnc2n1 ; Cn1cc2cc(-c3ccc4cc([C@H](O)C5(C)CC(F)(F)C5)sc4n3)cnc2n1 ; Cn1cc2cc(-c3ccc4cc([C@H](O)C56CC(C5)C6)sc4n3)cnc2n1 ; Cn1cc2cc(-c3ccc4cc([C@@H](O)C56CC(C5)C6)sc4n3)cnc2n1]

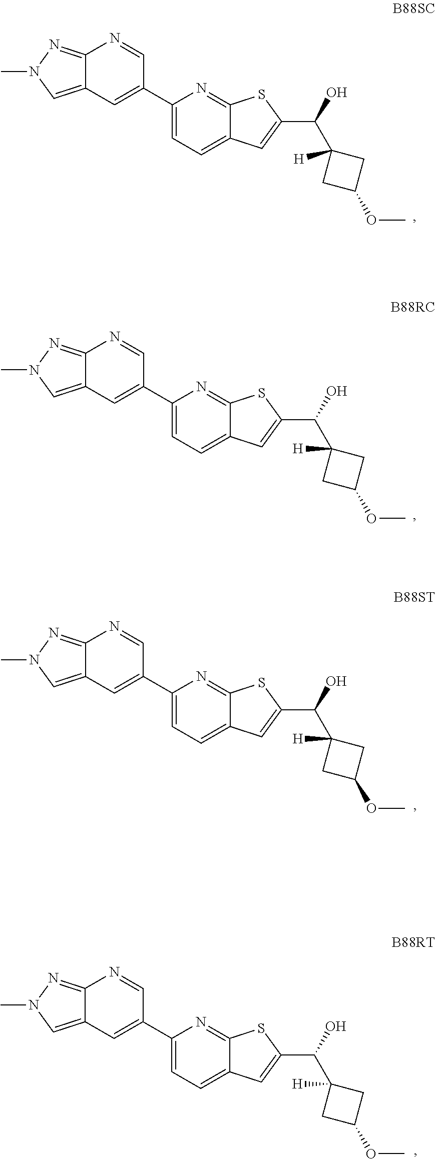

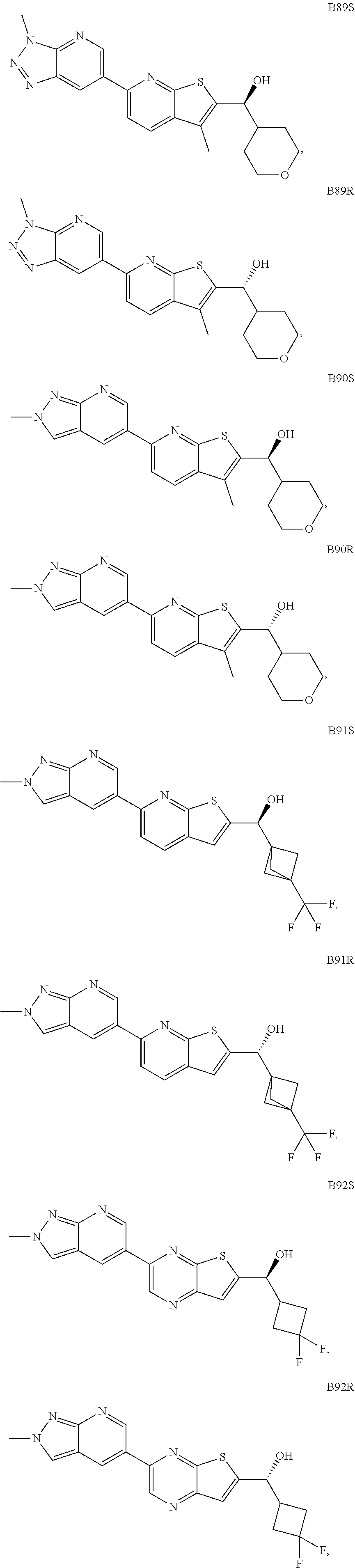

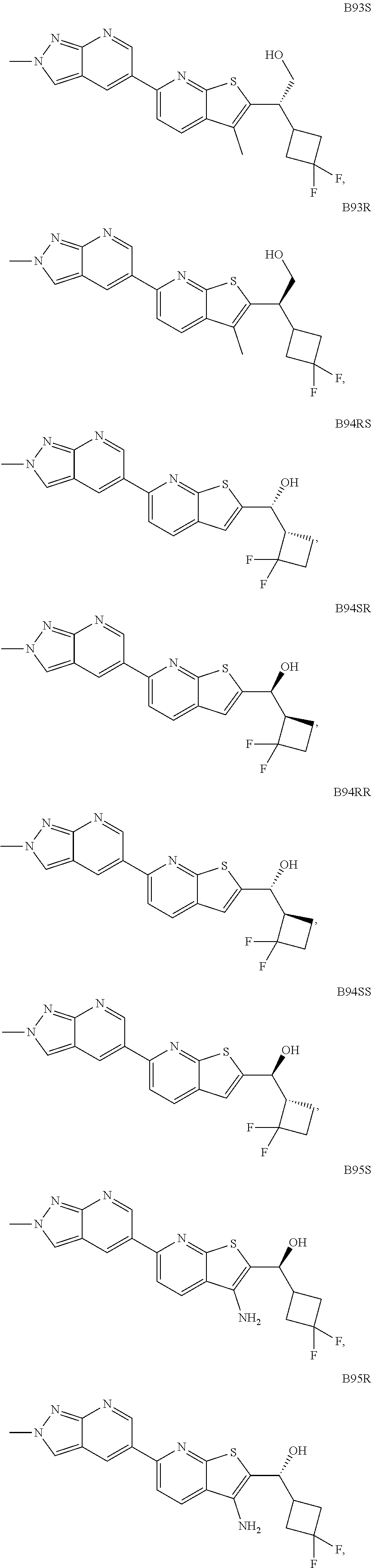

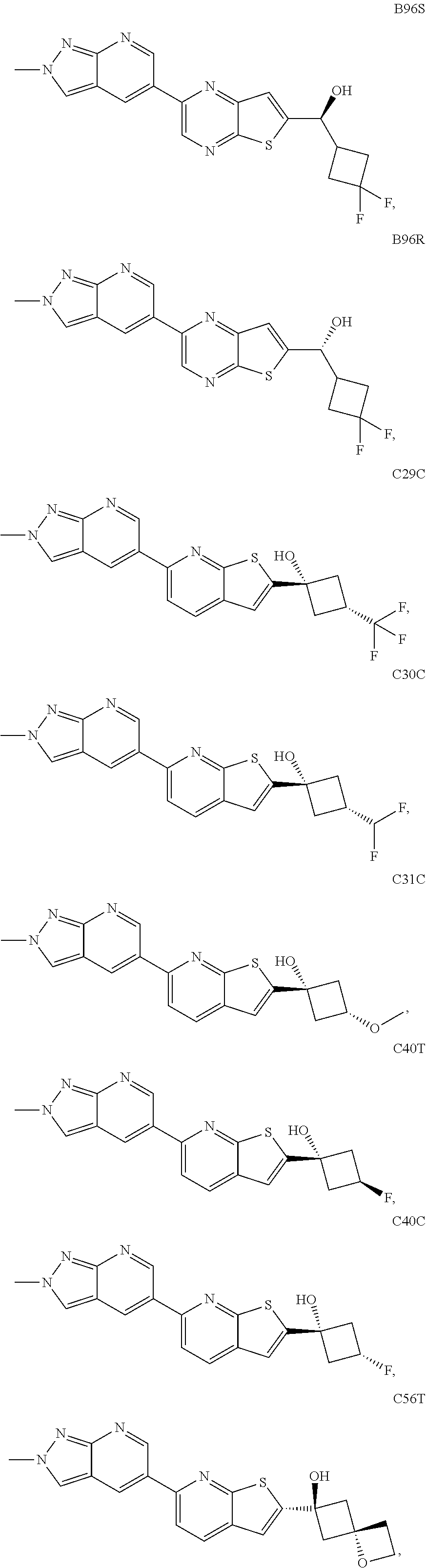

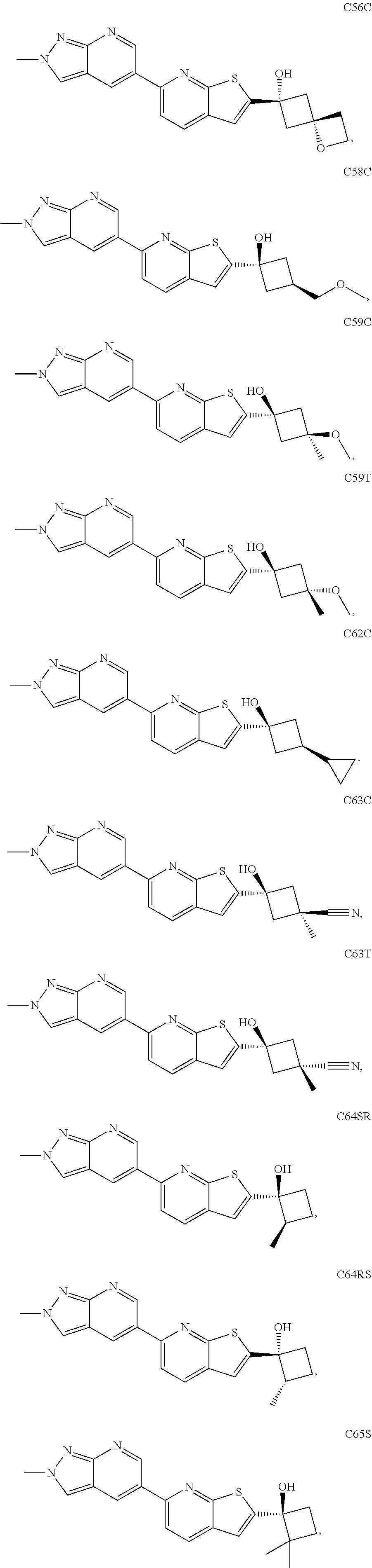

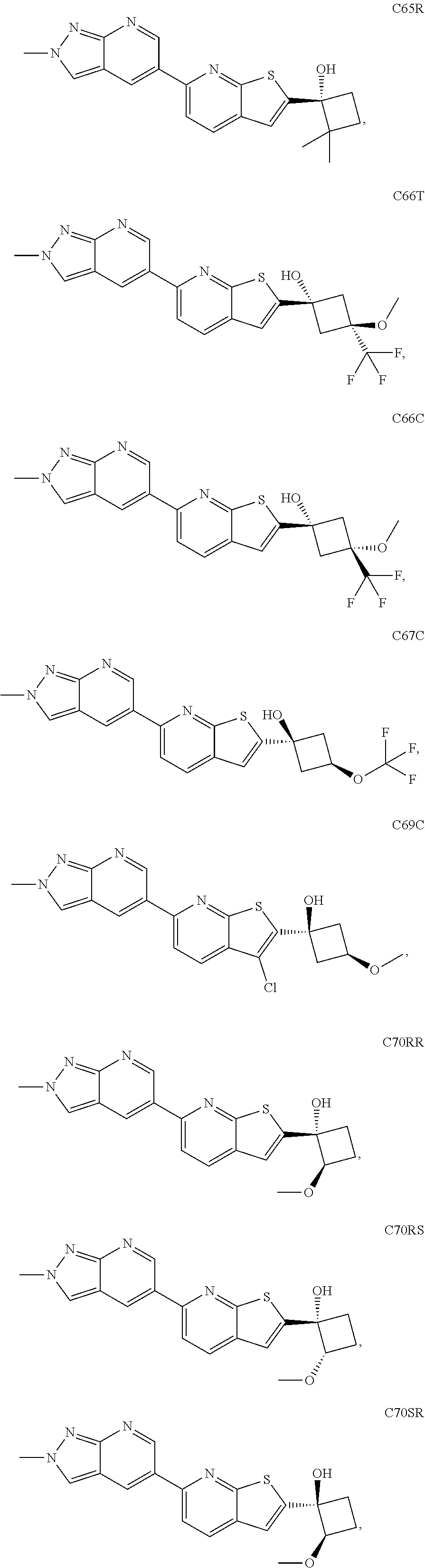

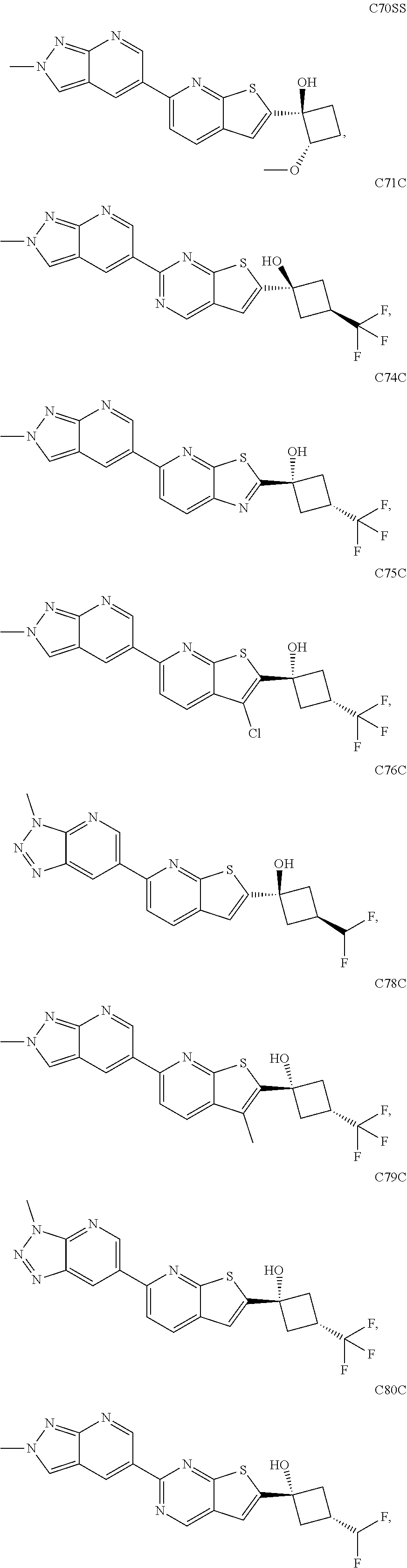

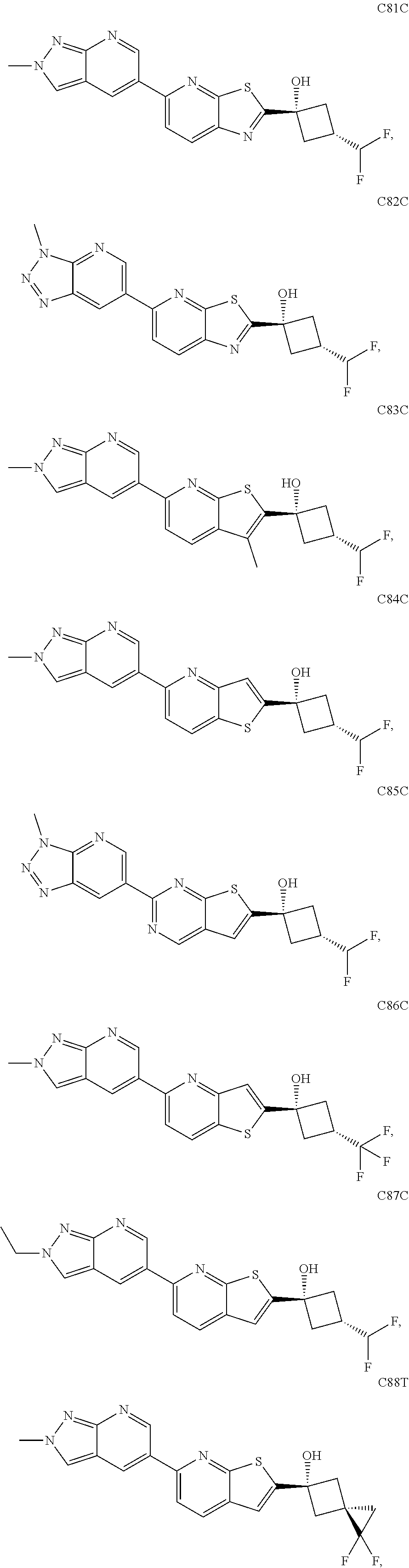

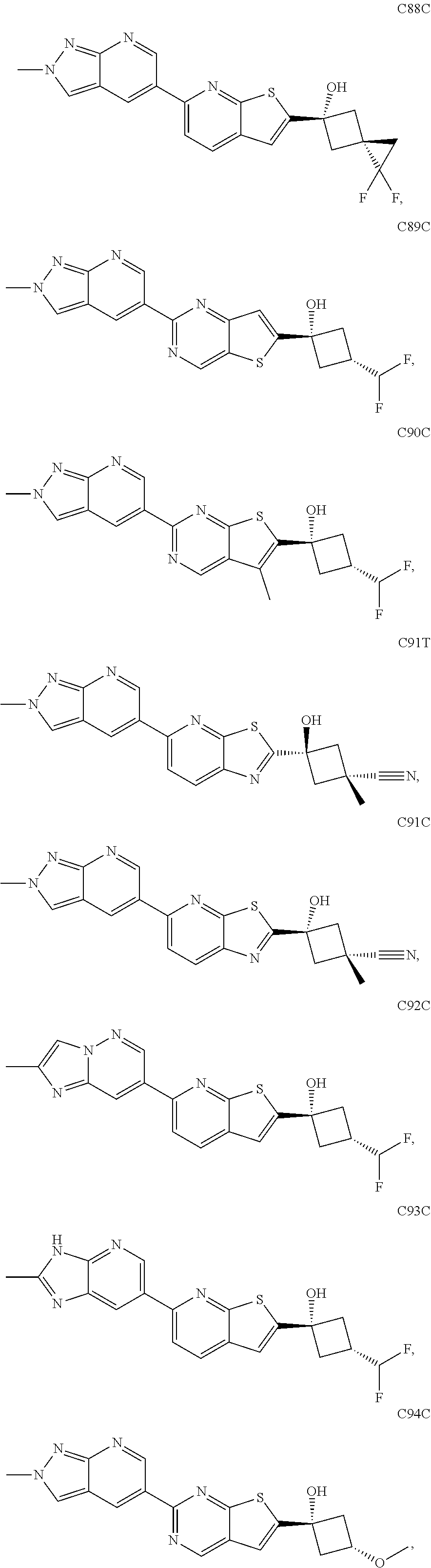

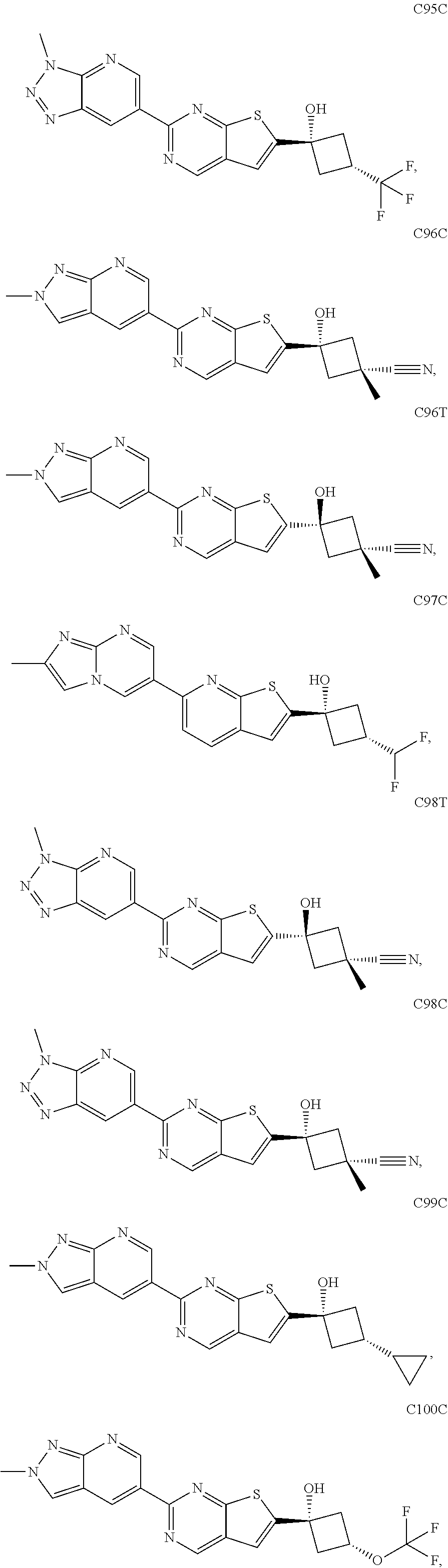

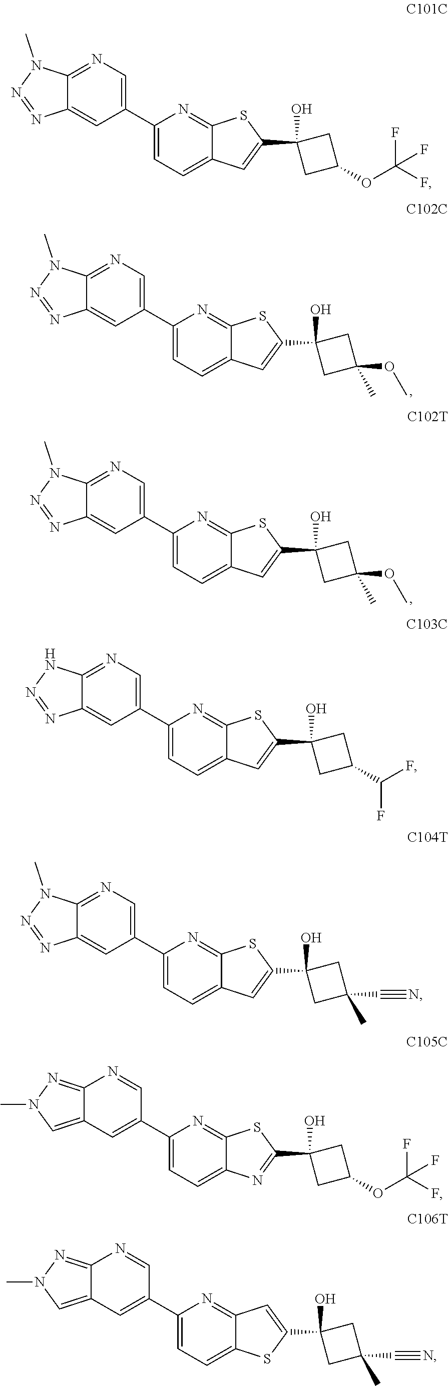

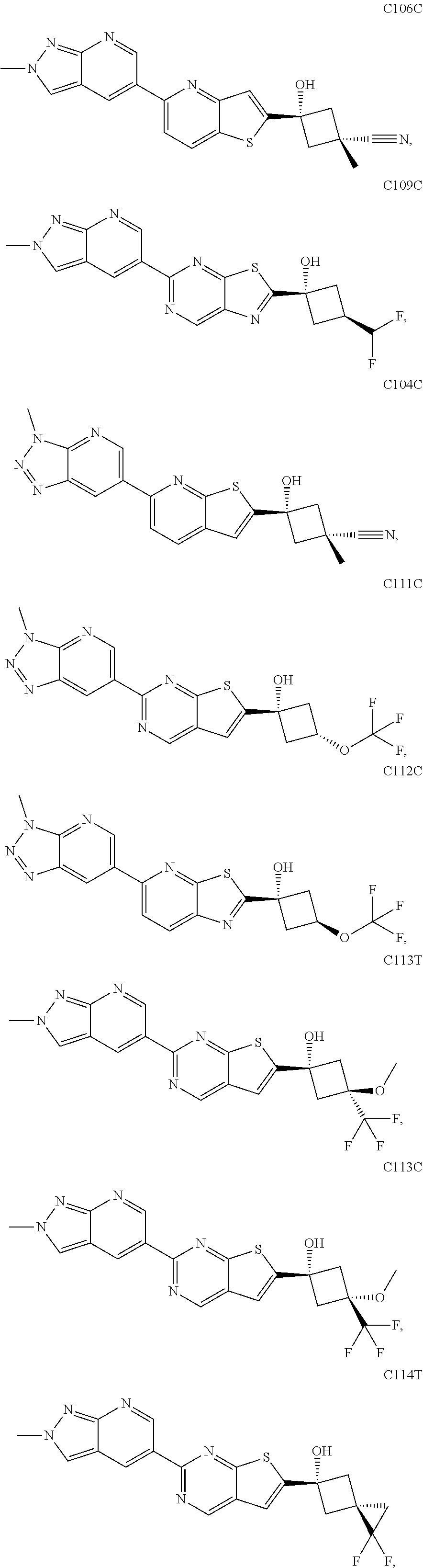

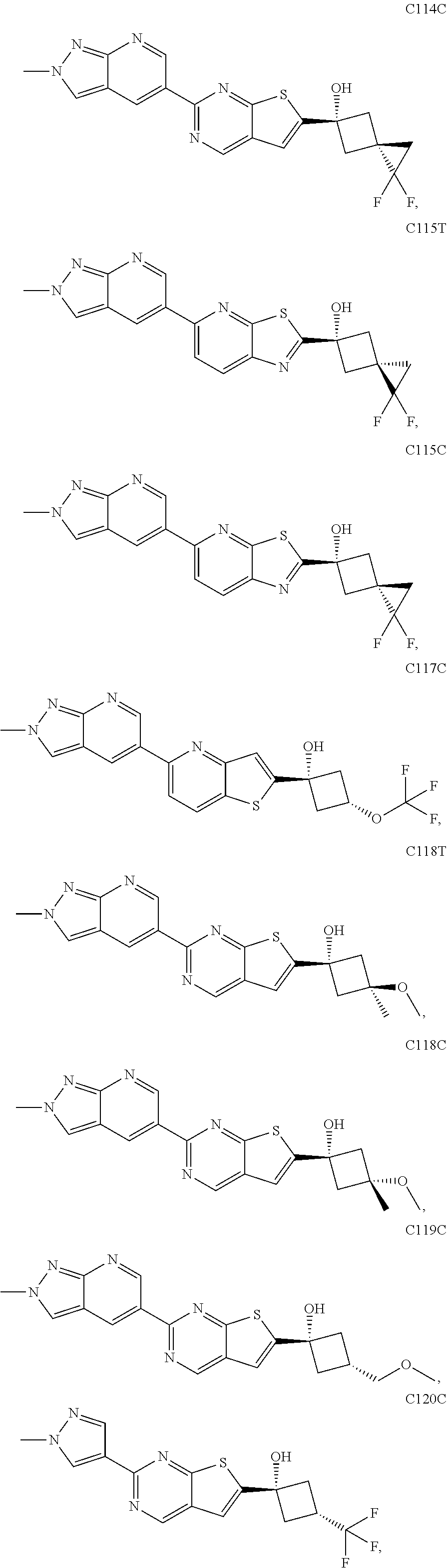

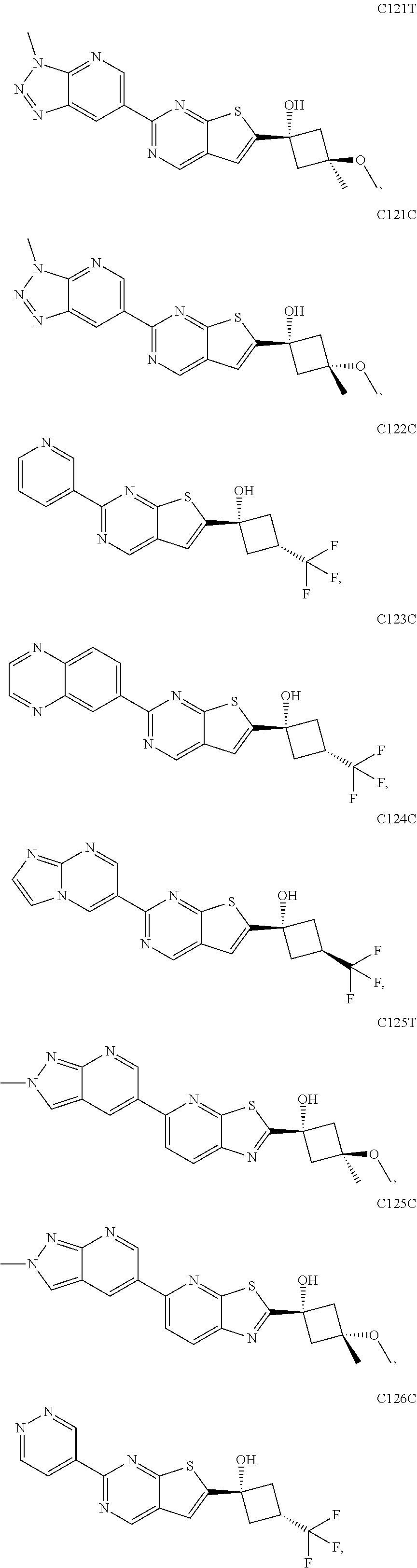

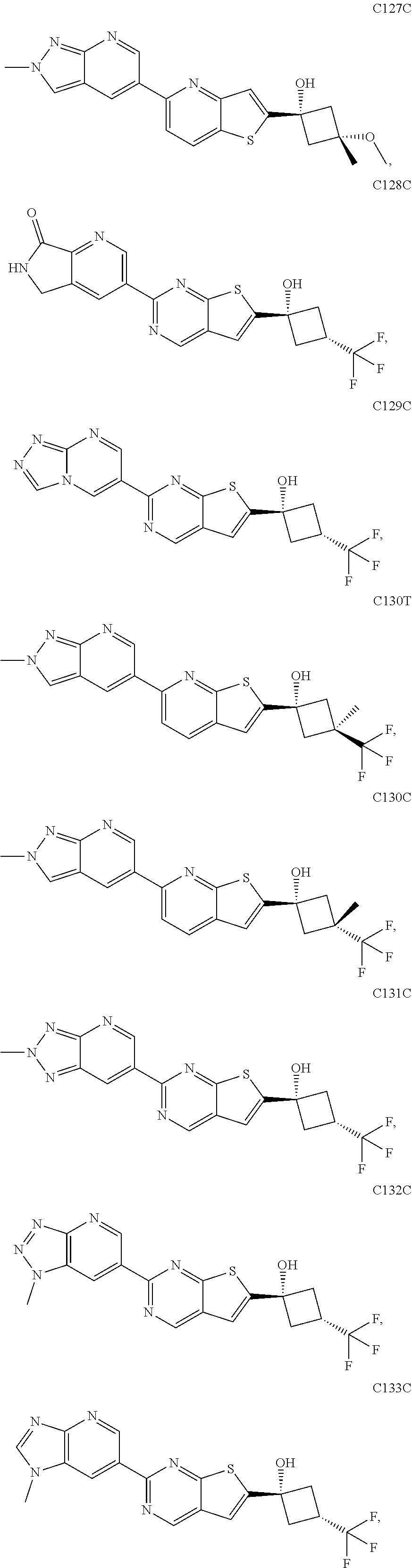

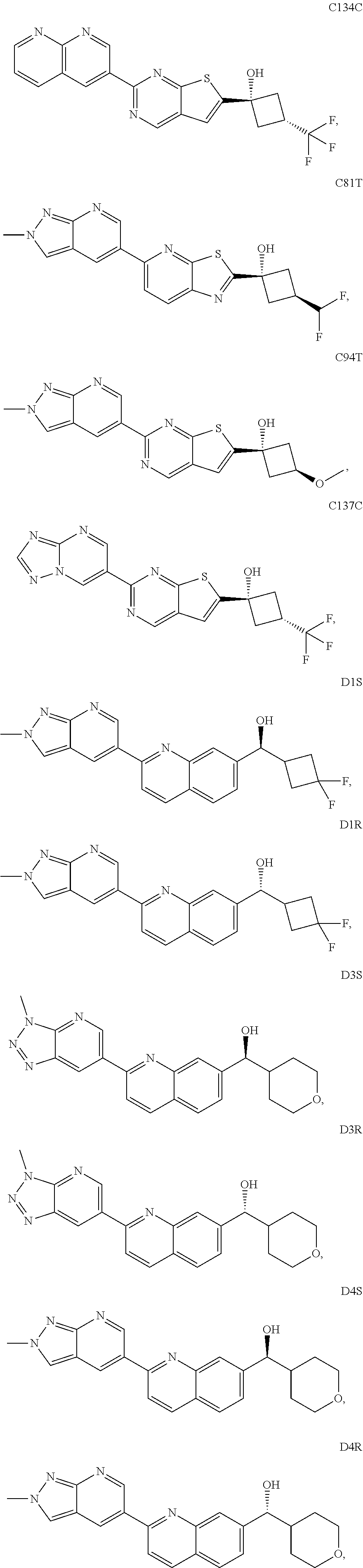

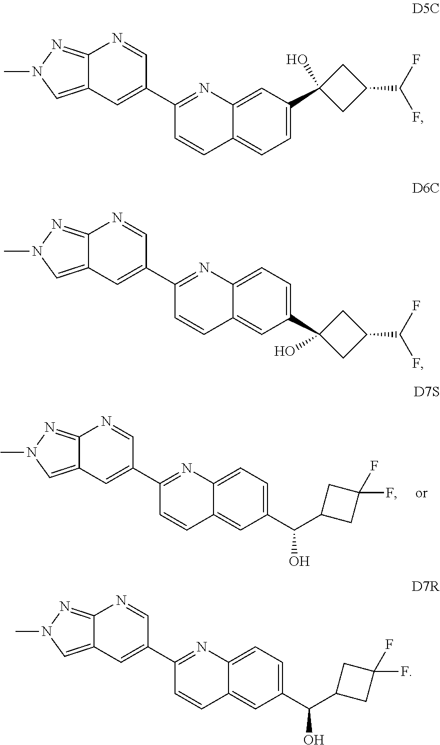 [SMILES: Cn1cc2cc(-c3ccc4ccc([C@]5(O)C[C@@H](C(F)F)C5)cc4n3)cnc2n1 ; Cn1cc2cc(-c3ccc4cc([C@]5(O)C[C@@H](C(F)F)C5)ccc4n3)cnc2n1 ; Cn1cc2cc(-c3ccc4cc([C@@H](O)C5CC(F)(F)C5)ccc4n3)cnc2n1 ; Cn1cc2cc(-c3ccc4cc([C@H](O)C5CC(F)(F)C5)ccc4n3)cnc2n1]

Provided herein as Embodiment 288 is the compound or salt of Embodiment 1, wherein the compound is:
5-(4-cyclobutyl-2-(methoxymethyl)thieno[2,3-b]pyridin-6-yl)-2-methyl-2H-pyrazolo[3,4-b]pyridine;
5-(2-(methoxymethyl)-4-(1-(methoxymethyl)cyclobutyl)thieno[2,3-b]pyridin-6-yl)-2-methyl-2H-pyrazolo[3,4-b]pyridine;
(R)-(4,6-bis(1-methyl-1H-pyrazol-5-yl)thieno[2,3-b]pyridin-2-yl)(cyclobutyl)methanol;
(S)-(4,6-bis(1-methyl-1H-pyrazol-5-yl)thieno[2,3-b]pyridin-2-yl)(cyclobutyl)methanol;
(R)-cyclobutyl(6-(2-methyl-2H-pyrazolo[3,4-b]pyridin-5-yl)-4-(1-methyl-1H-pyrazol-5-yl)thieno[2,3-b]pyridin-2-yl)methanol;
(S)-cyclobutyl(6-(2-methyl-2H-pyrazolo[3,4-b]pyridin-5-yl)-4-(1-methyl-1H-pyrazol-5-yl)thieno[2,3-b]pyridin-2-yl)methanol;
(R)-cyclobutyl(4-(2-methyl-2H-pyrazolo[3,4-b]pyridin-5-yl)-6-(1-methyl-1H-pyrazol-5-yl)thieno[2,3-b]pyridin-2-yl)methanol;
(S)-cyclobutyl(4-(2-methyl-2H-pyrazolo[3,4-b]pyridin-5-yl)-6-(1-methyl-1H-pyrazol-5-yl)thieno[2,3-b]pyridin-2-yl)methanol;
5-(2-((R)-cyclobutyl(methoxy)methyl)-4-(1-methyl-1H-pyrazol-5-yl)thieno[2,3-b]pyridin-6-yl)-2-methyl-2H-pyrazolo[3,4-b]pyridine;
5-(2-((S)-cyclobutyl(methoxy)methyl)-4-(1-methyl-1H-pyrazol-5-yl)thieno[2,3-b]pyridin-6-yl)-2-methyl-2H-pyrazolo [3,4-b]pyridine;
(1R)-1-cyclobutyl-1-(6-(2-methyl-2H-pyrazolo[3,4-b]pyridin-5-yl)-4-(1-methyl-1H-pyrazol-5-yl)thieno[2,3-b]pyridin-2-yl)ethanol;
(1S)-1-cyclobutyl-1-(6-(2-methyl-2H-pyrazolo[3,4-b]pyridin-5-yl)-4-(1-methyl-1H-pyrazol-5-yl)thieno[2,3-b]pyridin-2-yl)ethanol;
(1R)-1-cyclobutyl-2,2,2-trifluoro-1-(6-(2-methyl-2H-pyrazolo[3,4-b]pyridin-5-yl)-4-(1-methyl-1H-pyrazol-5-yl)thieno[2,3-b]pyridin-2-yl)ethanol;
(1S)-1-cyclobutyl-2,2,2-trifluoro-1-(6-(2-methyl-2H-pyrazolo[3,4-b]pyridin-5-yl)-4-(1-methyl-1H-pyrazol-5-yl)thieno[2,3-b]pyridin-2-yl)ethanol;
(1R)-1-(4-(2-methyl-2H-pyrazolo[3,4-b]pyridin-5-yl)-6-(1-methyl-1H-pyrazol-5-yl)thieno[2,3-b]pyridin-2-yl)ethanol;
(1S)-1-(4-(2-methyl-2H-pyrazolo[3,4-b]pyridin-5-yl)-6-(1-methyl-1H-pyrazol-5-yl)thieno[2,3-b]pyridin-2-yl)ethanol;
(1R)-1-(6-(2-methyl-2H-pyrazolo[3,4-b]pyridin-5-yl)-4-(1-methyl-1H-pyrazol-5-yl)thieno[2,3-b]pyridin-2-yl)ethanol;
(1S)-1-(6-(2-methyl-2H-pyrazolo[3,4-b]pyridin-5-yl)-4-(1-methyl-1H-pyrazol-5-yl)thieno[2,3-b]pyridin-2-yl)ethanol;
(R)-cyclobutyl(6-cyclopropyl-4-(2-methyl-2H-pyrazolo[3,4-b]pyridin-5-yl)thieno[2,3-b]pyridin-2-yl)methanol;
(S)-cyclobutyl(6-cyclopropyl-4-(2-methyl-2H-pyrazolo[3,4-b]pyridin-5-yl)thieno[2,3-b]pyridin-2-yl)methanol;
(R)-cyclobutyl(4-cyclopropyl-6-(2-methyl-2H-pyrazolo[3,4-b]pyridin-5-yl)thieno[2,3-b]pyridin-2-yl)methanol;
(S)-cyclobutyl(4-cyclopropyl-6-(2-methyl-2H-pyrazolo[3,4-b]pyridin-5-yl)thieno[2,3-b]pyridin-2-yl)methanol;
(4-(dimethylamino)-6-(2-methyl-2H-pyrazolo[3,4-b]pyridin-5-yl)thieno[2,3-b]pyridine-2,5-diyl)dimethanol;
(1R)-1-(4,6-bis(1-methyl-1H-pyrazol-5-yl)thieno[2,3-b]pyridin-2-yl)ethanol;
(1S)-1-(4,6-bis(1-methyl-1H-pyrazol-5-yl)thieno[2,3-b]pyridin-2-yl)ethanol;
(1R)-1-(4,6-bis(2-methyl-2H-pyrazolo[3,4-b]pyridin-5-yl)thieno[2,3-b]pyridin-2-yl)ethanol;
(1S)-1-(4,6-bis(2-methyl-2H-pyrazolo[3,4-b]pyridin-5-yl)thieno[2,3-b]pyridin-2-yl)ethanol;
(1R)-1-(4-cyclopropyl-6-(2-methyl-2H-pyrazolo[3,4-b]pyridin-5-yl)thieno[2,3-b]pyridin-2-yl)ethanol;
(1S)-1-(4-cyclopropyl-6-(2-methyl-2H-pyrazolo[3,4-b]pyridin-5-yl)thieno[2,3-b]pyridin-2-yl)ethanol;
(1R)-1-(6-cyclopropyl-4-(2-methyl-2H-pyrazolo[3,4-b]pyridin-5-yl)thieno[2,3-b]pyridin-2-yl)ethanol;
(1S)-1-(6-cyclopropyl-4-(2-methyl-2H-pyrazolo[3,4-b]pyridin-5-yl)thieno[2,3-b]pyridin-2-yl)ethanol;
(1R)-1-(4-cyclopropyl-6-(2-methyl-2H-pyrazolo[3,4-b]pyridin-5-yl)thieno[2,3-b]pyridin-2-yl)-1-propanol;
(1S)-1-(4-cyclopropyl-6-(2-methyl-2H-pyrazolo[3,4-b]pyridin-5-yl)thieno[2,3-b]pyridin-2-yl)-1-propanol;
(R)-(6-(2-methyl-2H-pyrazolo[3,4-b]pyridin-5-yl)thieno[2,3-b]pyridin-2-yl)(tetrahydro-2H-pyran-4-yl)methanol;
(S)-(6-(2-methyl-2H-pyrazolo[3,4-b]pyridin-5-yl)thieno[2,3-b]pyridin-2-yl)(tetrahydro-2H-pyran-4-yl)methanol;
(R)-cyclopropyl(6-(2-methyl-2H-pyrazolo[3,4-b]pyridin-5-yl)thieno[2,3-b]pyridin-2-yl)methanol;
(S)-cyclopropyl(6-(2-methyl-2H-pyrazolo[3,4-b]pyridin-5-yl)thieno[2,3-b]pyridin-2-yl)methanol;
(R)-cyclobutyl(6-(2-methyl-2H-pyrazolo[3,4-b]pyridin-5-yl)thieno[2,3-b]pyridin-2-yl)methanol;
(S)-cyclobutyl(6-(2-methyl-2H-pyrazolo[3,4-b]pyridin-5-yl)thieno[2,3-b]pyridin-2-yl)methanol;
(3R)-3-(6-(2-methyl-2H-pyrazolo[3,4-b]pyridin-5-yl)thieno[2,3-b]pyridin-2-yl)tetrahydro-3-furanol;
(3S)-3-(6-(2-methyl-2H-pyrazolo[3,4-b]pyridin-5-yl)thieno[2,3-b]pyridin-2-yl)tetrahydro-3-furanol;
1-(6-(2-methyl-2H-pyrazolo[3,4-b]pyridin-5-yl)thieno[2,3-b]pyridin-2-yl)cyclobutanol;
4-(6-(2-methyl-2H-pyrazolo[3,4-b]pyridin-5-yl)thieno[2,3-b]pyridin-2-yl)tetrahydro-2H-pyran-4-ol;
(R)-(6-(2-methyl-2H-pyrazolo[3,4-b]pyridin-5-yl)thieno[2,3-b]pyridin-2-yl)((3R)-tetrahydro-3-furanyl)methanol;
(R)-(6-(2-methyl-2H-pyrazolo[3,4-b]pyridin-5-yl)thieno[2,3-b]pyridin-2-yl)((3S)-tetrahydro-3-furanyl)methanol;
(S)-(6-(2-methyl-2H-pyrazolo[3,4-b]pyridin-5-yl)thieno[2,3-b]pyridin-2-yl)((3R)-tetrahydro-3-furanyl)methanol;
(S)-(6-(2-methyl-2H-pyrazolo[3,4-b]pyridin-5-yl)thieno[2,3-b]pyridin-2-yl)((3S)-tetrahydro-3-furanyl)methanol;
3-(6-(2-methyl-2H-pyrazolo[3,4-b]pyridin-5-yl)thieno[2,3-b]pyridin-2-yl)-3-oxetanol;
1-(6-(2-methyl-2H-pyrazolo[3,4-b]pyridin-5-yl)thieno[2,3-b]pyridin-2-yl)cyclopentanol;
(1R)-1-(6-(2-methyl-2H-pyrazolo[3,4-b]pyridin-5-yl)thieno[2,3-b]pyridin-2-yl)-1-propanol;
(1S)-1-(6-(2-methyl-2H-pyrazolo[3,4-b]pyridin-5-yl)thieno[2,3-b]pyridin-2-yl)-1-propanol;
(6-(2-methyl-2H-pyrazolo[3,4-b]pyridin-5-yl)thieno[2,3-b]pyridin-2-yl)methanol;
(1R)-2,2,2-trifluoro-1-(6-(2-methyl-2H-pyrazolo[3,4-b]pyridin-5-yl)thieno[2,3-b]pyridin-2-yl)ethanol;
(1S)-2,2,2-trifluoro-1-(6-(2-methyl-2H-pyrazolo[3,4-b]pyridin-5-yl)thieno[2,3-b]pyridin-2-yl)ethanol;
(1R)-2-methyl-1-(6-(2-methyl-2H-pyrazolo[3,4-b]pyridin-5-yl)thieno[2,3-b]pyridin-2-yl)-1-propanol;
(1S)-2-methyl-1-(6-(2-methyl-2H-pyrazolo[3,4-b]pyridin-5-yl)thieno[2,3-b]pyridin-2-yl)-1-propanol;
(1R)-1-(6-(2-methyl-2H-pyrazolo[3,4-b]pyridin-5-yl)thieno[2,3-b]pyridin-2-yl)ethanol;
(1S)-1-(6-(2-methyl-2H-pyrazolo[3,4-b]pyridin-5-yl)thieno[2,3-b]pyridin-2-yl)ethanol;
(1R)-1-(6-(2-methyl-2H-pyrazolo[3,4-b]pyridin-5-yl)thieno[2,3-b]pyridin-2-yl)-1-butanol;
(1S)-1-(6-(2-methyl-2H-pyrazolo[3,4-b]pyridin-5-yl)thieno[2,3-b]pyridin-2-yl)-1-butanol;
(R)-(6-(2-methyl-2H-pyrazolo[3,4-b]pyridin-5-yl)thieno[2,3-b]pyridin-2-yl)(3-oxetanyl)methanol;
(S)-(6-(2-methyl-2H-pyrazolo[3,4-b]pyridin-5-yl)thieno[2,3-b]pyridin-2-yl)(3-oxetanyl)methanol;
(1R)-3-methoxy-1-(6-(2-methyl-2H-pyrazolo[3,4-b]pyridin-5-yl)thieno[2,3-b]pyridin-2-yl)-1-propanol;
(1S)-3-methoxy-1-(6-(2-methyl-2H-pyrazolo[3,4-b]pyridin-5-yl)thieno[2,3-b]pyridin-2-yl)-1-propanol;
4,4-difluoro-1-(6-(2-methyl-2H-pyrazolo[3,4-b]pyridin-5-yl)thieno[2,3-b]pyridin-2-yl)cyclohexanol;
(2R)-1,1,1-trifluoro-3-methoxy-2-(6-(2-methyl-2H-pyrazolo[3,4-b]pyridin-5-yl)thieno[2,3-b]pyridin-2-yl)-2-propanol;
(2S)-1,1,1-trifluoro-3-methoxy-2-(6-(2-methyl-2H-pyrazolo[3,4-b]pyridin-5-yl)thieno[2,3-b]pyridin-2-yl)-2-propanol;
(1R)-1-cyclopropyl-2,2,2-trifluoro-1-(6-(2-methyl-2H-pyrazolo[3,4-b]pyridin-5-yl)thieno[2,3-b]pyridin-2-yl)ethanol;
(1S)-1-cyclopropyl-2,2,2-trifluoro-1-(6-(2-methyl-2H-pyrazolo[3,4-b]pyridin-5-yl)thieno[2,3-b]pyridin-2-yl)ethanol;
(R)-(3,3-difluorocyclobutyl)(6-(2-methyl-2H-pyrazolo[3,4-b]pyridin-5-yl)thieno[2,3-b]pyridin-2-yl)methanol;
(S)-(3,3-difluorocyclobutyl)(6-(2-methyl-2H-pyrazolo[3,4-b]pyridin-5-yl)thieno[2,3-b]pyridin-2-yl)methanol;
(R)-(4,4-difluorocyclohexyl)(6-(2-methyl-2H-pyrazolo[3,4-b]pyridin-5-yl)thieno[2,3-b]pyridin-2-yl)methanol;
(S)-(4,4-difluorocyclohexyl)(6-(2-methyl-2H-pyrazolo[3,4-b]pyridin-5-yl)thieno[2,3-b]pyridin-2-yl)methanol;
1-methyl-3-(6-(2-methyl-2H-pyrazolo[3,4-b]pyridin-5-yl)thieno[2,3-b]pyridin-2-yl)-3-azetidinol;
(3R)-1-methyl-3-(6-(2-methyl-2H-pyrazolo[3,4-b]pyridin-5-yl)thieno[2,3-b]pyridin-2-yl)-3-piperidinol;
(3S)-1-methyl-3-(6-(2-methyl-2H-pyrazolo[3,4-b]pyridin-5-yl)thieno[2,3-b]pyridin-2-yl)-3-piperidinol;
(2R)-1,1,1-trifluoro-2-(6-(2-methyl-2H-pyrazolo[3,4-b]pyridin-5-yl)thieno[2,3-b]pyridin-2-yl)-2-butanol;
(2S)-1,1,1-trifluoro-2-(6-(2-methyl-2H-pyrazolo[3,4-b]pyridin-5-yl)thieno[2,3-b]pyridin-2-yl)-2-butanol;
(1R)-2-methoxy-1-(6-(2-methyl-2H-pyrazolo[3,4-b]pyridin-5-yl)thieno[2,3-b]pyridin-2-yl)ethanol;
(1S)-2-methoxy-1-(6-(2-methyl-2H-pyrazolo[3,4-b]pyridin-5-yl)thieno[2,3-b]pyridin-2-yl)ethanol;
4-(6-(2-methyl-2H-pyrazolo[3,4-b]pyridin-5-yl)thieno[2,3-b]pyridin-2-yl)tetrahydro-2H-thiopyran-4-ol 1,1-dioxide;
2-(6-(2-methyl-2H-pyrazolo[3,4-b]pyridin-5-yl)thieno[2,3-b]pyridin-2-yl)-2-propanol;
(2R)-2-(6-(2-methyl-2H-pyrazolo[3,4-b]pyridin-5-yl)thieno[2,3-b]pyridin-2-yl)-2-butanol;
(2S)-2-(6-(2-methyl-2H-pyrazolo[3,4-b]pyridin-5-yl)thieno[2,3-b]pyridin-2-yl)-2-butanol;
(1R)-1-cyclopropyl-1-(6-(2-methyl-2H-pyrazolo[3,4-b]pyridin-5-yl)thieno[2,3-b]pyridin-2-yl)ethanol;
(1S)-1-cyclopropyl-1-(6-(2-methyl-2H-pyrazolo[3,4-b]pyridin-5-yl)thieno[2,3-b]pyridin-2-yl)ethanol;
1-methyl-4-(6-(2-methyl-2H-pyrazolo[3,4-b]pyridin-5-yl)thieno[2,3-b]pyridin-2-yl)-4-piperidinol;
(2R)-1-methoxy-2-(6-(2-methyl-2H-pyrazolo[3,4-b]pyridin-5-yl)thieno[2,3-b]pyridin-2-yl)-2-propanol;
(2S)-1-methoxy-2-(6-(2-methyl-2H-pyrazolo[3,4-b]pyridin-5-yl)thieno[2,3-b]pyridin-2-yl)-2-propanol;
3,3-difluoro-1-(6-(2-methyl-2H-pyrazolo[3,4-b]pyridin-5-yl)thieno[2,3-b]pyridin-2-yl)cyclobutanol;
(3R)-1-methyl-3-(6-(2-methyl-2H-pyrazolo[3,4-b]pyridin-5-yl)thieno[2,3-b]pyridin-2-yl)-3-pyrrolidinol;
(3S)-1-methyl-3-(6-(2-methyl-2H-pyrazolo[3,4-b]pyridin-5-yl)thieno[2,3-b]pyridin-2-yl)-3-pyrrolidinol;
(R)-(1-methyl-4-piperidinyl)(6-(2-methyl-2H-pyrazolo[3,4-b]pyridin-5-yl)thieno[2,3-b]pyridin-2-yl)methanol;
(S)-(1-methyl-4-piperidinyl)(6-(2-methyl-2H-pyrazolo[3,4-b]pyridin-5-yl)thieno[2,3-b]pyridin-2-yl)methanol;
(S)-(6-(1-methyl-1H-indazol-5-yl)thieno[2,3-b]pyridin-2-yl)(tetrahydro-2H-pyran-4-yl)methanol;
(R)-(6-(1-methyl-1H-indazol-5-yl)thieno[2,3-b]pyridin-2-yl)(tetrahydro-2H-pyran-4-yl)methanol;
(S)-(6-(1-methyl-1H-pyrazolo[3,4-b]pyridin-5-yl)thieno[2,3-b]pyridin-2-yl)(tetrahydro-2H-pyran-4-yl)methanol;
(R)-(6-(1-methyl-1H-pyrazolo[3,4-b]pyridin-5-yl)thieno[2,3-b]pyridin-2-yl)(tetrahydro-2H-pyran-4-yl)methanol;
(2R)-1,1,1-trifluoro-4-methoxy-2-(6-(2-methyl-2H-pyrazolo[3,4-b]pyridin-5-yl)thieno[2,3-b]pyridin-2-yl)-2-butanol;
(2S)-1,1,1-trifluoro-4-methoxy-2-(6-(2-methyl-2H-pyrazolo[3,4-b]pyridin-5-yl)thieno[2,3-b]pyridin-2-yl)-2-butanol;
(S)-(6-(1H-indazol-5-yl)thieno[2,3-b]pyridin-2-yl)(tetrahydro-2H-pyran-4-yl)methanol;
(R)-(6-(1H-indazol-5-yl)thieno[2,3-b]pyridin-2-yl)(tetrahydro-2H-pyran-4-yl)methanol;
(S)-(6-(6-methyl-1H-indazol-5-yl)thieno[2,3-b]pyridin-2-yl)(tetrahydro-2H-pyran-4-yl)methanol;
(R)-(6-(6-methyl-1H-indazol-5-yl)thieno[2,3-b]pyridin-2-yl)(tetrahydro-2H-pyran-4-yl)methanol;
(2R)-1,1,1-trifluoro-2-(6-(2-methyl-2H-pyrazolo[3,4-b]pyridin-5-yl)thieno[2,3-b]pyridin-2-yl)-2-propanol;
(2S)-1,1,1-trifluoro-2-(6-(2-methyl-2H-pyrazolo[3,4-b]pyridin-5-yl)thieno[2,3-b]pyridin-2-yl)-2-propanol;
(S)-(6-(2-methyl-2H-[1,2,3]triazolo[4,5-b]pyridin-6-yl)thieno[2,3-b]pyridin-2-yl)(tetrahydro-2H-pyran-4-yl)methanol;
(R)-(6-(2-methyl-2H-[1,2,3]triazolo[4,5-b]pyridin-6-yl)thieno[2,3-b]pyridin-2-yl)(tetrahydro-2H-pyran-4-yl)methanol;
(S)-(6-(3-methyl-1H-indazol-5-yl)thieno[2,3-b]pyridin-2-yl)(tetrahydro-2H-pyran-4-yl)methanol;
(R)-(6-(3-methyl-1H-indazol-5-yl)thieno[2,3-b]pyridin-2-yl)(tetrahydro-2H-pyran-4-yl)methanol;
(2R)-4-methoxy-2-(6-(2-methyl-2H-pyrazolo[3,4-b]pyridin-5-yl)thieno[2,3-b]pyridin-2-yl)-2-butanol;
(2S)-4-methoxy-2-(6-(2-methyl-2H-pyrazolo[3,4-b]pyridin-5-yl)thieno[2,3-b]pyridin-2-yl)-2-butanol;
(R)-(6-(3-methyl-3H-[1,2,3]triazolo[4,5-b]pyridin-6-yl)thieno[2,3-b]pyridin-2-yl)(tetrahydro-2H-pyran-4-yl)methanol;
(S)-(6-(3-methyl-3H-[1,2,3]triazolo[4,5-b]pyridin-6-yl)thieno[2,3-b]pyridin-2-yl)(tetrahydro-2H-pyran-4-yl)methanol;
(R)-(1-methyl-3-azetidinyl)(6-(2-methyl-2H-pyrazolo[3,4-b]pyridin-5-yl)thieno[2,3-b]pyridin-2-yl)methanol;
(S)-(1-methyl-3-azetidinyl)(6-(2-methyl-2H-pyrazolo[3,4-b]pyridin-5-yl)thieno[2,3-b]pyridin-2-yl)methanol;
(S)-(6-(1,2-benzoxazol-5-yl)thieno[2,3-b]pyridin-2-yl)(tetrahydro-2H-pyran-4-yl)methanol;
(R)-(6-(1,2-benzoxazol-5-yl)thieno[2,3-b]pyridin-2-yl)(tetrahydro-2H-pyran-4-yl)methanol;
(S)-tetrahydro-2H-pyran-4-yl(6-(3H-[1,2,3]triazolo[4,5-b]pyridin-6-yl)thieno[2,3-b]pyridin-2-yl)methanol;
(R)-tetrahydro-2H-pyran-4-yl(6-(3H-[1,2,3]triazolo[4,5-b]pyridin-6-yl)thieno[2,3-b]pyridin-2-yl)methanol;
(R)-((2R)-1-methyl-2-azetidinyl)(6-(2-methyl-2H-pyrazolo[3,4-b]pyridin-5-yl)thieno[2,3-b]pyridin-2-yl)methanol;
(R)-((2S)-1-methyl-2-azetidinyl)(6-(2-methyl-2H-pyrazolo[3,4-b]pyridin-5-yl)thieno[2,3-b]pyridin-2-yl)methanol;
(S)-((2R)-1-methyl-2-azetidinyl)(6-(2-methyl-2H-pyrazolo[3,4-b]pyridin-5-yl)thieno[2,3-b]pyridin-2-yl)methanol;
(S)-((2S)-1-methyl-2-azetidinyl)(6-(2-methyl-2H-pyrazolo[3,4-b]pyridin-5-yl)thieno[2,3-b]pyridin-2-yl)methanol;
(S)-(3,3-difluorocyclobutyl)(6-(2-methyl-1,3-benzoxazol-5-yl)thieno[2,3-b]pyridin-2-yl)methanol;
(R)-(3,3-difluorocyclobutyl)(6-(2-methyl-1,3-benzoxazol-5-yl)thieno[2,3-b]pyridin-2-yl)methanol;
(S)-(3,3-difluorocyclobutyl)(6-(1H-pyrazolo[3,4-b]pyridin-5-yl)thieno[2,3-b]pyridin-2-yl)methanol;
(R)-(3,3-difluorocyclobutyl)(6-(1H-pyrazolo[3,4-b]pyridin-5-yl)thieno[2,3-b]pyridin-2-yl)methanol;
(S)-(3,3-difluorocyclobutyl)(6-(2-methylimidazo[1,2-a]pyridin-6-yl)thieno[2,3-b]pyridin-2-yl)methanol;
(R)-(3,3-difluorocyclobutyl)(6-(2-methylimidazo[1,2-a]pyridin-6-yl)thieno[2,3-b]pyridin-2-yl)methanol;
6-(2-((R)-(3,3-difluorocyclobutyl)(hydroxy)methyl)thieno[2,3-b]pyridin-6-yl)-3-methyl-4(3H)-pyrimidinone;
6-(2-((S)-(3,3-difluorocyclobutyl)(hydroxy)methyl)thieno[2,3-b]pyridin-6-yl)-3-methyl-4(3H)-pyrimidinone;
6-(2-((S)-(3,3-difluorocyclobutyl)(hydroxy)methyl)thieno [2,3-b]pyridin-6-yl)-2-methyl-1(2H)-isoquinolinone;
6-(2-((R)-(3,3-difluorocyclobutyl)(hydroxy)methyl)thieno [2,3-b]pyridin-6-yl)-2-methyl-1(2H)-isoquinolinone;
(S)-(3,3-difluorocyclobutyl)(6-(2-methyl-1,3-benzoxazol-6-yl)thieno[2,3-b]pyridin-2-yl)methanol;
(R)-(3,3-difluorocyclobutyl)(6-(2-methyl-1,3-benzoxazol-6-yl)thieno[2,3-b]pyridin-2-yl)methanol;
(S)-(3,3-difluorocyclobutyl)(6-(6-quinoxalinyl)thieno[2,3-b]pyridin-2-yl)methanol;
(R)-(3,3-difluorocyclobutyl)(6-(6-quinoxalinyl)thieno[2,3-b]pyridin-2-yl)methanol;
(S)-(3,3-difluorocyclobutyl)(6-(2-ethyl-2H-pyrazolo[3,4-b]pyridin-5-yl)thieno[2,3-b]pyridin-2-yl)methanol;
(R)-(3,3-difluorocyclobutyl)(6-(2-ethyl-2H-pyrazolo[3,4-b]pyridin-5-yl)thieno[2,3-b]pyridin-2-yl)methanol;
(1S)-1-(6-(2-methyl-2H-pyrazolo[3,4-b]pyridin-5-yl)-1-benzothiophen-2-yl)-1-butanol;
(1R)-1-(6-(2-methyl-2H-pyrazolo[3,4-b]pyridin-5-yl)-1-benzothiophen-2-yl)-1-butanol;
(S)-(3,3-difluorocyclobutyl)(6-(2-methylimidazo[1,2-a]pyridin-7-yl)thieno[2,3-b]pyridin-2-yl)methanol;
(R)-(3,3-difluorocyclobutyl)(6-(2-methylimidazo[1,2-a]pyridin-7-yl)thieno[2,3-b]pyridin-2-yl)methanol;
(S)-(3,3-difluorocyclobutyl)(6-(2-(2-propanyl)-2H-indazol-5-yl)thieno[2,3-b]pyridin-2-yl)methanol;
(R)-(3,3-difluorocyclobutyl)(6-(2-(2-propanyl)-2H-indazol-5-yl)thieno[2,3-b]pyridin-2-yl)methanol;
(S)-(3,3-difluorocyclobutyl)(6-(5-pyrimidinyl)thieno[2,3-b]pyridin-2-yl)methanol;
(R)-(3,3-difluorocyclobutyl)(6-(5-pyrimidinyl)thieno[2,3-b]pyridin-2-yl)methanol;
(S)-cyclopentyl(6-(2-methyl-2H-pyrazolo[3,4-b]pyridin-5-yl)-1-benzothiophen-2-yl)methanol;
(R)-cyclopentyl(6-(2-methyl-2H-pyrazolo[3,4-b]pyridin-5-yl)-1-benzothiophen-2-yl)methanol;
(1S)-3-methoxy-1-(6-(2-methyl-2H-pyrazolo[3,4-b]pyridin-5-yl)-1-benzothiophen-2-yl)-1-propanol;
(1R)-3-methoxy-1-(6-(2-methyl-2H-pyrazolo[3,4-b]pyridin-5-yl)-1-benzothiophen-2-yl)-1-propanol;
(1S)-4,4,4-trifluoro-1-(6-(2-methyl-2H-pyrazolo[3,4-b]pyridin-5-yl)-1-benzothiophen-2-yl)-1-butanol;
(1R)-4,4,4-trifluoro-1-(6-(2-methyl-2H-pyrazolo[3,4-b]pyridin-5-yl)-1-benzothiophen-2-yl)-1-butanol;
(S)-(3,3-difluorocyclobutyl)(6-(2-methyl-2H-pyrazolo[3,4-b]pyridin-5-yl)-1-benzothiophen-2-yl)methanol;
(R)-(3,3-difluorocyclobutyl)(6-(2-methyl-2H-pyrazolo[3,4-b]pyridin-5-yl)-1-benzothiophen-2-yl)methanol;
(R)-(6-(2-methyl-2H-pyrazolo[3,4-b]pyridin-5-yl)thieno[2,3-b]pyridin-2-yl)((3R)-1-methyl-3-pyrrolidinyl)methanol;
(R)-(6-(2-methyl-2H-pyrazolo[3,4-b]pyridin-5-yl)thieno[2,3-b]pyridin-2-yl)((3S)-1-methyl-3-pyrrolidinyl)methanol;
(S)-(6-(2-methyl-2H-pyrazolo[3,4-b]pyridin-5-yl)thieno[2,3-b]pyridin-2-yl)((3R)-1-methyl-3-pyrrolidinyl)methanol;
(S)-(6-(2-methyl-2H-pyrazolo[3,4-b]pyridin-5-yl)thieno[2,3-b]pyridin-2-yl)((3S)-1-methyl-3-pyrrolidinyl)methanol;
(R)-((3R)-1-methyl-3-piperidinyl)(6-(2-methyl-2H-pyrazolo[3,4-b]pyridin-5-yl)thieno[2,3-b]pyridin-2-yl)methanol;
(R)-((3S)-1-methyl-3-piperidinyl)(6-(2-methyl-2H-pyrazolo[3,4-b]pyridin-5-yl)thieno[2,3-b]pyridin-2-yl)methanol;
(S)-((3R)-1-methyl-3-piperidinyl)(6-(2-methyl-2H-pyrazolo[3,4-b]pyridin-5-yl)thieno[2,3-b]pyridin-2-yl)methanol;
(S)-((3S)-1-methyl-3-piperidinyl)(6-(2-methyl-2H-pyrazolo[3,4-b]pyridin-5-yl)thieno[2,3-b]pyridin-2-yl)methanol;
(S)-(3,3-difluorocyclobutyl)(6-(2-(2-propanyl)-2H-pyrazolo[3,4-b]pyridin-5-yl)thieno[2,3-b]pyridin-2-yl)methanol;
(R)-(3,3-difluorocyclobutyl)(6-(2-(2-propanyl)-2H-pyrazolo[3,4-b]pyridin-5-yl)thieno[2,3-b]pyridin-2-yl)methanol;
(S)-(3,3-difluorocyclobutyl)(6-(1-(2-propanyl)-1H-pyrazolo[3,4-b]pyridin-5-yl)thieno[2,3-b]pyridin-2-yl)methanol;
(R)-(3,3-difluorocyclobutyl)(6-(1-(2-propanyl)-1H-pyrazolo[3,4-b]pyridin-5-yl)thieno[2,3-b]pyridin-2-yl)methanol;
(R)-(6-(2-methyl-2H-pyrazolo[3,4-b]pyridin-5-yl)thieno[2,3-b]pyridin-2-yl)(tetrahydro-2H-pyran-4-yl)methan-d-ol;
(S)-(6-(2-methyl-2H-pyrazolo[3,4-b]pyridin-5-yl)thieno[2,3-b]pyridin-2-yl)(tetrahydro-2H-pyran-4-yl)methan-d-ol;
(R)-(3-amino-6-(5-pyrimidinyl)thieno[2,3-b]pyridin-2-yl)(3,3-difluorocyclobutyl)methanol;
(S)-(3-amino-6-(5-pyrimidinyl)thieno[2,3-b]pyridin-2-yl)(3,3-difluorocyclobutyl)methanol;
2-(6-(2-methyl-2H-pyrazolo[3,4-b]pyridin-5-yl)thieno[2,3-b]pyridin-2-yl)spiro[3.3]heptan-2-ol;
3,3-dimethyl-1-(6-(2-methyl-2H-pyrazolo[3,4-b]pyridin-5-yl)thieno[2,3-b]pyridin-2-yl)cyclobutanol;
6,6-difluoro-2-(6-(2-methyl-2H-pyrazolo[3,4-b]pyridin-5-yl)thieno[2,3-b]pyridin-2-yl)spiro[3.3]heptan-2-ol;
6-(6-(2-methyl-2H-pyrazolo[3,4-b]pyridin-5-yl)thieno[2,3-b]pyridin-2-yl)-2-oxaspiro[3.3]heptan-6-ol;
cis-1-(6-(2-methyl-2H-pyrazolo[3,4-b]pyridin-5-yl)thieno[2,3-b]pyridin-2-yl)-3-(trifluoromethyl)cyclobutanol;
cis-3-(difluoromethyl)-1-(6-(2-methyl-2H-pyrazolo[3,4-b]pyridin-5-yl)thieno[2,3-b]pyridin-2-yl)cyclobutanol;
cis-3-methoxy-1-(6-(2-methyl-2H-pyrazolo[3,4-b]pyridin-5-yl)thieno[2,3-b]pyridin-2-yl)cyclobutanol;
(S)-(3,3-difluorocyclobutyl)(5-(2-methyl-2H-pyrazolo[3,4-b]pyridin-5-yl)[1,3]thiazolo[5,4-b]pyridin-2-yl)methanol;
(R)-(3,3-difluorocyclobutyl)(5-(2-methyl-2H-pyrazolo[3,4-b]pyridin-5-yl)[1,3]thiazolo[5,4-b]pyridin-2-yl)methanol;
1-(6-(2-ethyl-2H-pyrazolo[3,4-b]pyridin-5-yl)thieno[2,3-b]pyridin-2-yl)-3,3-difluorocyclobutanol;
(S)-(3,3-difluorocyclobutyl)(5-(2-methyl-2H-pyrazolo[3,4-b]pyridin-5-yl)thieno[3,2-b]pyridin-2-yl)methanol;
(R)-(3,3-difluorocyclobutyl)(5-(2-methyl-2H-pyrazolo[3,4-b]pyridin-5-yl)thieno[3,2-b]pyridin-2-yl)methanol;
3,3-difluoro-1-(6-(3-methyl-3H-imidazo[4,5-b]pyridin-6-yl)thieno[2,3-b]pyridin-2-yl)cyclobutanol;
3,3-difluoro-1-(6-(2-methyl[1,3]oxazolo[4,5-b]pyridin-6-yl)thieno[2,3-b]pyridin-2-yl)cyclobutanol;
3,3-difluoro-1-(6-(2-methyl-3H-imidazo[4,5-b]pyridin-6-yl)thieno[2,3-b]pyridin-2-yl)cyclobutanol;
3,3-difluoro-1-(6-(1H-pyrazolo[3,4-b]pyridin-5-yl)thieno[2,3-b]pyridin-2-yl)cyclobutanol;
3,3-difluoro-1-(6-(2-methylimidazo[1,2-a]pyridin-6-yl)thieno[2,3-b]pyridin-2-yl)cyclobutanol;
1-(6-(2-amino-5-pyrimidinyl)thieno[2,3-b]pyridin-2-yl)-3,3-difluorocyclobutanol;
(S)-(3,3-difluorocyclobutyl)(5-(2-methyl-2H-pyrazolo[3,4-b]pyridin-5-yl)[1,3]thiazolo[4,5-b]pyridin-2-yl)methanol;
(R)-(3,3-difluorocyclobutyl)(5-(2-methyl-2H-pyrazolo[3,4-b]pyridin-5-yl)[1,3]thiazolo[4,5-b]pyridin-2-yl)methanol;
3,3-difluoro-1-(6-(2-methyl-1,3-benzoxazol-6-yl)thieno[2,3-b]pyridin-2-yl)cyclobutanol;
trans-3-fluoro-1-(6-(2-methyl-2H-pyrazolo[3,4-b]pyridin-5-yl)thieno[2,3-b]pyridin-2-yl)cyclobutanol;
(R)-(3,3-difluorocyclobutyl)(2-(2-methyl-2H-pyrazolo[3,4-b]pyridin-5-yl)thieno[2,3-d]pyrimidin-6-yl)methanol;
(S)-(3,3-difluorocyclobutyl)(2-(2-methyl-2H-pyrazolo[3,4-b]pyridin-5-yl)thieno[2,3-d]pyrimidin-6-yl)methanol;
(S)-(3-chloro-6-(2-methyl-2H-pyrazolo[3,4-b]pyridin-5-yl)thieno[2,3-b]pyridin-2-yl)(3,3-difluorocyclobutyl)methanol;
(R)-(3-chloro-6-(2-methyl-2H-pyrazolo[3,4-b]pyridin-5-yl)thieno[2,3-b]pyridin-2-yl)(3,3-difluorocyclobutyl)methanol;
3,3-difluoro-1-(6-(3H-imidazo[4,5-b]pyridin-6-yl)thieno[2,3-b]pyridin-2-yl)cyclobutanol;
3,3-difluoro-1-(6-(1H-indazol-5-yl)thieno[2,3-b]pyridin-2-yl)cyclobutanol;
(R)-(3,3-difluorocyclobutyl)(5-(2-methyl-2H-pyrazolo[3,4-b]pyridin-5-yl)thieno[2,3-b]pyridin-2-yl)methanol;
(S)-(3,3-difluorocyclobutyl)(5-(2-methyl-2H-pyrazolo[3,4-b]pyridin-5-yl)thieno[2,3-b]pyridin-2-yl)methanol;
(S)-(3,3-difluorocyclobutyl)(6-(2-methyl-2H-pyrazolo[3,4-b]pyridin-5-yl)thieno[3,2-b]pyridin-2-yl)methanol;
(R)-(3,3-difluorocyclobutyl)(6-(2-methyl-2H-pyrazolo[3,4-b]pyridin-5-yl)thieno[3,2-b]pyridin-2-yl)methanol;
3,3-difluoro-1-(6-(2-(methylamino)-5-pyrimidinyl)thieno[2,3-b]pyridin-2-yl)cyclobutanol;
(R)-(3,3-difluorocyclobutyl)(6-(2-methyl-2H-pyrazolo[3,4-b]pyridin-5-yl)thieno[2,3-b]pyridin-2-yl)methan-d-ol;
(S)-(3,3-difluorocyclobutyl)(6-(2-methyl-2H-pyrazolo[3,4-b]pyridin-5-yl)thieno[2,3-b]pyridin-2-yl)methan-d-ol;
3,3-difluoro-1-(6-(2-methylimidazo[1,2-a]pyridin-7-yl)thieno[2,3-b]pyridin-2-yl)cyclobutanol;
3,3-difluoro-1-(6-(2-methylimidazo[1,2-a]pyrimidin-6-yl)thieno[2,3-b]pyridin-2-yl)cyclobutanol;
(2R)-2-(3,3-difluorocyclobutyl)-2-(6-(2-methyl-2H-pyrazolo[3,4-b]pyridin-5-yl)thieno[2,3-b]pyridin-2-yl)ethanol;
(2S)-2-(3,3-difluorocyclobutyl)-2-(6-(2-methyl-2H-pyrazolo[3,4-b]pyridin-5-yl)thieno[2,3-b]pyridin-2-yl)ethanol;
3,3-difluoro-1-(6-(5-pyrimidinyl)thieno[2,3-b]pyridin-2-yl)cyclobutanol;
3,3-difluoro-1-(6-(pyrido[2,3-b]pyrazin-7-yl)thieno[2,3-b]pyridin-2-yl)cyclobutanol;
(S)-(3,3-difluorocyclobutyl)(2-(2-methyl-2H-pyrazolo[3,4-b]pyridin-5-yl)-7-quinolinyl)methanol;
(R)-(3,3-difluorocyclobutyl)(2-(2-methyl-2H-pyrazolo[3,4-b]pyridin-5-yl)-7-quinolinyl)methanol;
3,3-difluoro-1-(6-(2-methyl-2H-pyrazolo[3,4-b]pyridin-5-yl)-1-benzothiophen-2-yl)cyclobutanol;
3,3-difluoro-1-(6-(2-methyl-2H-indazol-5-yl)thieno[2,3-b]pyridin-2-yl)cyclobutanol;
1-(6-(1,2-benzoxazol-5-yl)thieno[2,3-b]pyridin-2-yl)-3,3-difluorocyclobutanol;
3,3-difluoro-1-(6-(3-methyl-3H-[1,2,3]triazolo[4,5-b]pyridin-6-yl)thieno[2,3-b]pyridin-2-yl)cyclobutanol;
2-(6-(2-methyl-2H-pyrazolo[3,4-b]pyridin-5-yl)thieno[2,3-b]pyridin-2-yl)spiro[3.5]nonan-2-ol;
2-(6-(2-methyl-2H-pyrazolo[3,4-b]pyridin-5-yl)thieno[2,3-b]pyridin-2-yl)spiro[3.4]octan-2-ol;
3,3-difluoro-1-(6-(2-methylimidazo[1,2-b]pyridazin-7-yl)thieno[2,3-b]pyridin-2-yl)cyclobutanol;
3,3-difluoro-1-(6-(3H-[1,2,3]triazolo[4,5-b]pyridin-6-yl)thieno[2,3-b]pyridin-2-yl)cyclobutanol;
(R)-(3,3-difluorocyclobutyl)(6-(3-methyl-3H-[1,2,3]triazolo[4,5-b]pyridin-6-yl)thieno[2,3-b]pyridin-2-yl)methanol;
(S)-(3,3-difluorocyclobutyl)(6-(3-methyl-3H-[1,2,3]triazolo[4,5-b]pyridin-6-yl)thieno[2,3-b]pyridin-2-yl)methanol;
trans-6-(6-(2-methyl-2H-pyrazolo[3,4-b]pyridin-5-yl)thieno[2,3-b]pyridin-2-yl)-1-oxaspiro[3.3]heptan-6-ol;
cis-6-(6-(2-methyl-2H-pyrazolo[3,4-b]pyridin-5-yl)thieno[2,3-b]pyridin-2-yl)-1-oxaspiro[3.3]heptan-6-ol;
3,3-dimethyl-1-(2-(2-methyl-2H-pyrazolo[3,4-b]pyridin-5-yl)thieno[2,3-d]pyrimidin-6-yl)cyclobutanol;
cis-3-(methoxymethyl)-1-(6-(2-methyl-2H-pyrazolo[3,4-b]pyridin-5-yl)thieno[2,3-b]pyridin-2-yl)cyclobutanol;
trans-3-methoxy-3-methyl-1-(6-(2-methyl-2H-pyrazolo[3,4-b]pyridin-5-yl)thieno[2,3-b]pyridin-2-yl)cyclobutanol;
cis-3-methoxy-3-methyl-1-(6-(2-methyl-2H-pyrazolo[3,4-b]pyridin-5-yl)thieno[2,3-b]pyridin-2-yl)cyclobutanol;
(R)-(3,3-difluorocyclobutyl)(3-methyl-6-(2-methyl-2H-pyrazolo[3,4-b]pyridin-5-yl)thieno[2,3-b]pyridin-2-yl)methanol;
(S)-(3,3-difluorocyclobutyl)(3-methyl-6-(2-methyl-2H-pyrazolo[3,4-b]pyridin-5-yl)thieno[2,3-b]pyridin-2-yl)methanol;
1-(6-(2-methyl-2H-pyrazolo[3,4-b]pyridin-5-yl)thieno[2,3-b]pyridin-2-yl)cyclopropanol;
5-(6-(2-methyl-2H-pyrazolo[3,4-b]pyridin-5-yl)thieno[2,3-b]pyridin-2-yl)spiro[2.3]hexan-5-ol;
cis-3-cyclopropyl-1-(6-(2-methyl-2H-pyrazolo [3,4-b]pyridin-5-yl)thieno [2,3-b]pyridin-2-yl)cyclobutanol;
trans-3-hydroxy-1-methyl-3-(6-(2-methyl-2H-pyrazolo[3,4-b]pyridin-5-yl)thieno[2,3-b]pyridin-2-yl)cyclobutanecarbonitrile;
cis-3-hydroxy-1-methyl-3-(6-(2-methyl-2H-pyrazolo[3,4-b]pyridin-5-yl)thieno[2,3-b]pyridin-2-yl)cyclobutanecarbonitrile;
(1S,2R)-2-methyl-1-(6-(2-methyl-2H-pyrazolo[3,4-b]pyridin-5-yl)thieno[2,3-b]pyridin-2-yl)cyclobutanol;
(1R,2S)-2-methyl-1-(6-(2-methyl-2H-pyrazolo[3,4-b]pyridin-5-yl)thieno[2,3-b]pyridin-2-yl)cyclobutanol;
(1S)-2,2-dimethyl-1-(6-(2-methyl-2H-pyrazolo[3,4-b]pyridin-5-yl)thieno[2,3-b]pyridin-2-yl)cyclobutanol;
(1R)-2,2-dimethyl-1-(6-(2-methyl-2H-pyrazolo[3,4-b]pyridin-5-yl)thieno[2,3-b]pyridin-2-yl)cyclobutanol;
trans-3-methoxy-1-(6-(2-methyl-2H-pyrazolo[3,4-b]pyridin-5-yl)thieno[2,3-b]pyridin-2-yl)-3-(trifluoromethyl)cyclobutanol;
cis-3-methoxy-1-(6-(2-methyl-2H-pyrazolo[3,4-b]pyridin-5-yl)thieno[2,3-b]pyridin-2-yl)-3-(trifluoromethyl)cyclobutanol;
cis-1-(6-(2-methyl-2H-pyrazolo[3,4-b]pyridin-5-yl)thieno[2,3-b]pyridin-2-yl)-3-(trifluoromethoxy)cyclobutanol;
3,3-dimethyl-1-(5-(2-methyl-2H-pyrazolo[3,4-b]pyridin-5-yl)[1,3]thiazolo[5,4-b]pyridin-2-yl)cyclobutanol;
cis-1-(3-chloro-6-(2-methyl-2H-pyrazolo[3,4-b]pyridin-5-yl)thieno[2,3-b]pyridin-2-yl)-3-methoxycyclobutanol;
(1R,2R)-2-methoxy-1-(6-(2-methyl-2H-pyrazolo[3,4-b]pyridin-5-yl)thieno[2,3-b]pyridin-2-yl)cyclobutanol;
(1R,2S)-2-methoxy-1-(6-(2-methyl-2H-pyrazolo[3,4-b]pyridin-5-yl)thieno[2,3-b]pyridin-2-yl)cyclobutanol;
(1S,2R)-2-methoxy-1-(6-(2-methyl-2H-pyrazolo[3,4-b]pyridin-5-yl)thieno[2,3-b]pyridin-2-yl)cyclobutanol;
cis-1-(2-(2-methyl-2H-pyrazolo[3,4-b]pyridin-5-yl)thieno[2,3-d]pyrimidin-6-yl)-3-(trifluoromethyl)cyclobutanol;
(1S,2S)-2-methoxy-1-(6-(2-methyl-2H-pyrazolo[3,4-b]pyridin-5-yl)thieno[2,3-b]pyridin-2-yl)cyclobutanol;
1-(3-chloro-6-(2-methyl-2H-pyrazolo[3,4-b]pyridin-5-yl)thieno[2,3-b]pyridin-2-yl)-3,3-dimethylcyclobutanol;
cis-1-(5-(2-methyl-2H-pyrazolo[3,4-b]pyridin-5-yl)[1,3]thiazolo[5,4-b]pyridin-2-yl)-3-(trifluoromethyl)cyclobutanol;
(S)-(6-(2-methyl-2H-pyrazolo[3,4-b]pyridin-5-yl)thieno[2,3-b]pyridin-2-yl)((cis)-3-(trifluoromethyl)cyclobutyl)methanol;
(R)-(6-(2-methyl-2H-pyrazolo[3,4-b]pyridin-5-yl)thieno[2,3-b]pyridin-2-yl)((cis)-3-(trifluoromethyl)cyclobutyl)methanol;
(S)-(6-(2-methyl-2H-pyrazolo[3,4-b]pyridin-5-yl)thieno[2,3-b]pyridin-2-yl)((trans)-3-(trifluoromethyl)cyclobutyl)methanol;
(R)-(6-(2-methyl-2H-pyrazolo[3,4-b]pyridin-5-yl)thieno[2,3-b]pyridin-2-yl)((trans)-3-(trifluoromethyl)cyclobutyl)methanol;
cis-1-(3-chloro-6-(2-methyl-2H-pyrazolo[3,4-b]pyridin-5-yl)thieno[2,3-b]pyridin-2-yl)-3-(trifluoromethyl)cyclobutanol;
(R)-((1R)-2,2-difluorocyclopropyl)(6-(2-methyl-2H-pyrazolo[3,4-b]pyridin-5-yl)thieno[2,3-b]pyridin-2-yl)methanol;
(S)-((1S)-2,2-difluorocyclopropyl)(6-(2-methyl-2H-pyrazolo[3,4-b]pyridin-5-yl)thieno[2,3-b]pyridin-2-yl)methanol;
(S)-((1R)-2,2-difluorocyclopropyl)(6-(2-methyl-2H-pyrazolo[3,4-b]pyridin-5-yl)thieno[2,3-b]pyridin-2-yl)methanol;
(R)-((1S)-2,2-difluorocyclopropyl)(6-(2-methyl-2H-pyrazolo[3,4-b]pyridin-5-yl)thieno[2,3-b]pyridin-2-yl)methanol;
(R)-(3,3-difluoro-1-methylcyclobutyl)(6-(2-methyl-2H-pyrazolo[3,4-b]pyridin-5-yl)thieno[2,3-b]pyridin-2-yl)methanol;
(S)-(3,3-difluoro-1-methylcyclobutyl)(6-(2-methyl-2H-pyrazolo[3,4-b]pyridin-5-yl)thieno[2,3-b]pyridin-2-yl)methanol;
(R)-bicyclo[1.1.1]pentan-1-yl(6-(2-methyl-2H-pyrazolo[3,4-b]pyridin-5-yl)thieno[2,3-b]pyridin-2-yl)methanol;
(S)-bicyclo[1.1.1]pentan-1-yl(6-(2-methyl-2H-pyrazolo[3,4-b]pyridin-5-yl)thieno[2,3-b]pyridin-2-yl)methanol;
(S)-((cis)-3-methoxycyclobutyl)(6-(2-methyl-2H-pyrazolo[3,4-b]pyridin-5-yl)thieno[2,3-b]pyridin-2-yl)methanol;
cis-3-(difluoromethyl)-1-(6-(3-methyl-3H-[1,2,3]triazolo[4,5-b]pyridin-6-yl)thieno[2,3-b]pyridin-2-yl)cyclobutanol;
(R)-((cis)-3-methoxycyclobutyl)(6-(2-methyl-2H-pyrazolo[3,4-b]pyridin-5-yl)thieno[2,3-b]pyridin-2-yl)methanol;
(S)-((trans)-3-methoxycyclobutyl)(6-(2-methyl-2H-pyrazolo[3,4-b]pyridin-5-yl)thieno[2,3-b]pyridin-2-yl)methanol;
(R)-((trans)-3-methoxycyclobutyl)(6-(2-methyl-2H-pyrazolo[3,4-b]pyridin-5-yl)thieno[2,3-b]pyridin-2-yl)methanol;
3,3-dimethyl-1-(2-(2-methyl-2H-pyrazolo[3,4-b]pyridin-5-yl)-7-quinolinyl)cyclobutanol;
3,3-difluoro-1-(3-methyl-6-(2-methyl-2H-pyrazolo[3,4-b]pyridin-5-yl)thieno[2,3-b]pyridin-2-yl)cyclobutanol;
cis-1-(3-methyl-6-(2-methyl-2H-pyrazolo[3,4-b]pyridin-5-yl)thieno[2,3-b]pyridin-2-yl)-3-(trifluoromethyl)cyclobutanol;
cis-1-(6-(3-methyl-3H-[1,2,3]triazolo[4,5-b]pyridin-6-yl)thieno[2,3-b]pyridin-2-yl)-3-(trifluoromethyl)cyclobutanol;
cis-3-(difluoromethyl)-1-(2-(2-methyl-2H-pyrazolo[3,4-b]pyridin-5-yl)thieno[2,3-d]pyrimidin-6-yl)cyclobutanol;
(R)-(2-(3-methyl-3H-[1,2,3]triazolo[4,5-b]pyridin-6-yl)-7-quinolinyl)(tetrahydro-2H-pyran-4-yl)methanol;
(S)-(2-(3-methyl-3H-[1,2,3]triazolo[4,5-b]pyridin-6-yl)-7-quinolinyl)(tetrahydro-2H-pyran-4-yl)methanol;
(R)-(2-(2-methyl-2H-pyrazolo[3,4-b]pyridin-5-yl)-7-quinolinyl)(tetrahydro-2H-pyran-4-yl)methanol;
(S)-(2-(2-methyl-2H-pyrazolo[3,4-b]pyridin-5-yl)-7-quinolinyl)(tetrahydro-2H-pyran-4-yl)methanol;
cis-3-(difluoromethyl)-1-(5-(2-methyl-2H-pyrazolo[3,4-b]pyridin-5-yl)[1,3]thiazolo[5,4-b]pyridin-2-yl)cyclobutanol;
cis-3-(difluoromethyl)-1-(5-(3-methyl-3H-[1,2,3]triazolo[4,5-b]pyridin-6-yl)[1,3]thiazolo[5,4-b]pyridin-2-yl)cyclobutanol;
(R)-(3-methyl-6-(3-methyl-3H-[1,2,3]triazolo[4,5-b]pyridin-6-yl)thieno[2,3-b]pyridin-2-yl)(tetrahydro-2H-pyran-4-yl)methanol;
(S)-(3-methyl-6-(3-methyl-3H-[1,2,3]triazolo[4,5-b]pyridin-6-yl)thieno[2,3-b]pyridin-2-yl)(tetrahydro-2H-pyran-4-yl)methanol;
(R)-(3-methyl-6-(2-methyl-2H-pyrazolo[3,4-b]pyridin-5-yl)thieno[2,3-b]pyridin-2-yl)(tetrahydro-2H-pyran-4-yl)methanol;
(S)-(3-methyl-6-(2-methyl-2H-pyrazolo[3,4-b]pyridin-5-yl)thieno[2,3-b]pyridin-2-yl)(tetrahydro-2H-pyran-4-yl)methanol;
cis-3-(difluoromethyl)-1-(2-(2-methyl-2H-pyrazolo[3,4-b]pyridin-5-yl)-7-quinolinyl)cyclobutanol;
(R)-(6-(2-methyl-2H-pyrazolo[3,4-b]pyridin-5-yl)thieno[2,3-b]pyridin-2-yl)(3-(trifluoromethyl)bicyclo[1.1.1]pentan-1-yl)methanol;
(S)-(6-(2-methyl-2H-pyrazolo[3,4-b]pyridin-5-yl)thieno[2,3-b]pyridin-2-yl)(3-(trifluoromethyl)bicyclo[1.1.1]pentan-1-yl)methanol;
cis-3-fluoro-1-(6-(2-methyl-2H-pyrazolo[3,4-b]pyridin-5-yl)thieno[2,3-b]pyridin-2-yl)cyclobutanol;
cis-3-(difluoromethyl)-1-(3-methyl-6-(2-methyl-2H-pyrazolo[3,4-b]pyridin-5-yl)thieno[2,3-b]pyridin-2-yl)cyclobutanol;
cis-3-(difluoromethyl)-1-(2-(2-methyl-2H-pyrazolo[3,4-b]pyridin-5-yl)-6-quinolinyl)cyclobutanol;
cis-3-(difluoromethyl)-1-(5-(2-methyl-2H-pyrazolo[3,4-b]pyridin-5-yl)thieno[3,2-b]pyridin-2-yl)cyclobutanol;
cis-3-(difluoromethyl)-1-(2-(3-methyl-3H-[1,2,3]triazolo[4,5-b]pyridin-6-yl)thieno[2,3-d]pyrimidin-6-yl)cyclobutanol;
cis-1-(5-(2-methyl-2H-pyrazolo[3,4-b]pyridin-5-yl)thieno[3,2-b]pyridin-2-yl)-3-(trifluoromethyl)cyclobutanol;
(S)-(3,3-difluorocyclobutyl)(2-(2-methyl-2H-pyrazolo[3,4-b]pyridin-5-yl)-6-quinolinyl)methanol;
(R)-(3,3-difluorocyclobutyl)(2-(2-methyl-2H-pyrazolo[3,4-b]pyridin-5-yl)-6-quinolinyl)methanol;
cis-3-(difluoromethyl)-1-(6-(2-ethyl-2H-pyrazolo[3,4-b]pyridin-5-yl)thieno[2,3-b]pyridin-2-yl)cyclobutanol;
(trans)-1,1-difluoro-5-(6-(2-methyl-2H-pyrazolo[3,4-b]pyridin-5-yl)thieno[2,3-b]pyridin-2-yl)spiro[2.3]hexan-5-ol;
(cis)-1,1-difluoro-5-(6-(2-methyl-2H-pyrazolo[3,4-b]pyridin-5-yl)thieno[2,3-b]pyridin-2-yl)spiro[2.3]hexan-5-ol;
cis-3-(difluoromethyl)-1-(2-(2-methyl-2H-pyrazolo[3,4-b]pyridin-5-yl)thieno[3,2-d]pyrimidin-6-yl)cyclobutanol;
cis-3-(difluoromethyl)-1-(5-methyl-2-(2-methyl-2H-pyrazolo[3,4-b]pyridin-5-yl)thieno[2,3-d]pyrimidin-6-yl)cyclobutanol;
trans-3-hydroxy-1-methyl-3-(5-(2-methyl-2H-pyrazolo[3,4-b]pyridin-5-yl)[1,3]thiazolo[5,4-b]pyridin-2-yl)cyclobutanecarbonitrile;
cis-3-hydroxy-1-methyl-3-(5-(2-methyl-2H-pyrazolo[3,4-b]pyridin-5-yl)[1,3]thiazolo[5,4-b]pyridin-2-yl)cyclobutanecarbonitrile;
cis-3-(difluoromethyl)-1-(6-(2-methylimidazo[1,2-b]pyridazin-7-yl)thieno[2,3-b]pyridin-2-yl)cyclobutanol;
cis-3-(difluoromethyl)-1-(6-(2-methyl-3H-imidazo[4,5-b]pyridin-6-yl)thieno[2,3-b]pyridin-2-yl)cyclobutanol;
(R)-(3,3-difluorocyclobutyl)(3-(2-methyl-2H-pyrazolo[3,4-b]pyridin-5-yl)thieno[2,3-b]pyrazin-6-yl)methanol;
(S)-(3,3-difluorocyclobutyl)(3-(2-methyl-2H-pyrazolo[3,4-b]pyridin-5-yl)thieno[2,3-b]pyrazin-6-yl)methanol;
(2S)-2-(3,3-difluorocyclobutyl)-2-(3-methyl-6-(2-methyl-2H-pyrazolo[3,4-b]pyridin-5-yl)thieno[2,3-b]pyridin-2-yl)ethanol;
(2R)-2-(3,3-difluorocyclobutyl)-2-(3-methyl-6-(2-methyl-2H-pyrazolo[3,4-b]pyridin-5-yl)thieno[2,3-b]pyridin-2-yl)ethanol;
cis-3-methoxy-1-(2-(2-methyl-2H-pyrazolo[3,4-b]pyridin-5-yl)thieno[2,3-d]pyrimidin-6-yl)cyclobutanol;
cis-1-(2-(3-methyl-3H-[1,2,3]triazolo[4,5-b]pyridin-6-yl)thieno[2,3-d]pyrimidin-6-yl)-3-(trifluoromethyl)cyclobutanol;
cis-3-hydroxy-1-methyl-3-(2-(2-methyl-2H-pyrazolo[3,4-b]pyridin-5-yl)thieno[2,3-d]pyrimidin-6-yl)cyclobutanecarbonitrile;
trans-3-hydroxy-1-methyl-3-(2-(2-methyl-2H-pyrazolo[3,4-b]pyridin-5-yl)thieno[2,3-d]pyrimidin-6-yl)cyclobutanecarbonitrile;
cis-3-(difluoromethyl)-1-(6-(2-methylimidazo[1,2-a]pyrimidin-6-yl)thieno[2,3-b]pyridin-2-yl)cyclobutanol;
trans-3-hydroxy-1-methyl-3-(2-(3-methyl-3H-[1,2,3]triazolo[4,5-b]pyridin-6-yl)thieno[2,3-d]pyrimidin-6-yl)cyclobutanecarbonitrile;
cis-3-hydroxy-1-methyl-3-(2-(3-methyl-3H-[1,2,3]triazolo[4,5-b]pyridin-6-yl)thieno[2,3-d]pyrimidin-6-yl)cyclobutanecarbonitrile;
cis-3-cyclopropyl-1-(2-(2-methyl-2H-pyrazolo[3,4-b]pyridin-5-yl)thieno[2,3-d]pyrimidin-6-yl)cyclobutanol;
cis-1-(2-(2-methyl-2H-pyrazolo[3,4-b]pyridin-5-yl)thieno[2,3-d]pyrimidin-6-yl)-3-(trifluoromethoxy)cyclobutanol;
cis-1-(6-(3-methyl-3H-[1,2,3]triazolo[4,5-b]pyridin-6-yl)thieno[2,3-b]pyridin-2-yl)-3-(trifluoromethoxy)cyclobutanol;
cis-3-methoxy-3-methyl-1-(6-(3-methyl-3H-[1,2,3]triazolo[4,5-b]pyridin-6-yl)thieno[2,3-b]pyridin-2-yl)cyclobutanol;
cis-3-(difluoromethyl)-1-(6-(3H-[1,2,3]triazolo[4,5-b]pyridin-6-yl)thieno[2,3-b]pyridin-2-yl)cyclobutanol;
trans-3-hydroxy-1-methyl-3-(6-(3-methyl-3H-[1,2,3]triazolo[4,5-b]pyridin-6-yl)thieno[2,3-b]pyridin-2-yl)cyclobutanecarbonitrile;
cis-1-(5-(2-methyl-2H-pyrazolo[3,4-b]pyridin-5-yl)[1,3]thiazolo[5,4-b]pyridin-2-yl)-3-(trifluoromethoxy)cyclobutanol;
trans-3-hydroxy-1-methyl-3-(5-(2-methyl-2H-pyrazolo[3,4-b]pyridin-5-yl)thieno[3,2-b]pyridin-2-yl)cyclobutanecarbonitrile;
cis-3-hydroxy-1-methyl-3-(5-(2-methyl-2H-pyrazolo[3,4-b]pyridin-5-yl)thieno[3,2-b]pyridin-2-yl)cyclobutanecarbonitrile;
5-(2-(2-methyl-2H-pyrazolo[3,4-b]pyridin-5-yl)thieno[2,3-d]pyrimidin-6-yl)spiro[2.3]hexan-5-ol;
5-(5-(2-methyl-2H-pyrazolo[3,4-b]pyridin-5-yl)[1,3]thiazolo[5,4-b]pyridin-2-yl)spiro[2.3]hexan-5-ol;
(R)-((1S)-2,2-difluorocyclobutyl)(6-(2-methyl-2H-pyrazolo[3,4-b]pyridin-5-yl)thieno[2,3-b]pyridin-2-yl)methanol;
(S)-((1R)-2,2-difluorocyclobutyl)(6-(2-methyl-2H-pyrazolo[3,4-b]pyridin-5-yl)thieno[2,3-b]pyridin-2-yl)methanol;
(R)-((1R)-2,2-difluorocyclobutyl)(6-(2-methyl-2H-pyrazolo[3,4-b]pyridin-5-yl)thieno[2,3-b]pyridin-2-yl)methanol;
(S)-((1S)-2,2-difluorocyclobutyl)(6-(2-methyl-2H-pyrazolo[3,4-b]pyridin-5-yl)thieno[2,3-b]pyridin-2-yl)methanol;
cis-3-(difluoromethyl)-1-(5-(2-methyl-2H-pyrazolo[3,4-b]pyridin-5-yl)[1,3]thiazolo[5,4-d]pyrimidin-2-yl)cyclobutanol;
cis-3-hydroxy-1-methyl-3-(6-(3-methyl-3H-[1,2,3]triazolo[4,5-b]pyridin-6-yl)thieno[2,3-b]pyridin-2-yl)cyclobutanecarbonitrile;
cis-1-(2-(3-methyl-3H-[1,2,3]triazolo[4,5-b]pyridin-6-yl)thieno[2,3-d]pyrimidin-6-yl)-3-(trifluoromethoxy)cyclobutanol;
cis-1-(5-(3-methyl-3H-[1,2,3]triazolo[4,5-b]pyridin-6-yl)[1,3]thiazolo[5,4-b]pyridin-2-yl)-3-(trifluoromethoxy)cyclobutanol;
trans-3-methoxy-1-(2-(2-methyl-2H-pyrazolo[3,4-b]pyridin-5-yl)thieno[2,3-d]pyrimidin-6-yl)-3-(trifluoromethyl)cyclobutanol;
cis-3-methoxy-1-(2-(2-methyl-2H-pyrazolo[3,4-b]pyridin-5-yl)thieno[2,3-d]pyrimidin-6-yl)-3-(trifluoromethyl)cyclobutanol;
trans-1,1-difluoro-5-(2-(2-methyl-2H-pyrazolo[3,4-b]pyridin-5-yl)thieno[2,3-d]pyrimidin-6-yl)spiro[2.3]hexan-5-ol;
cis-1,1-difluoro-5-(2-(2-methyl-2H-pyrazolo[3,4-b]pyridin-5-yl)thieno[2,3-d]pyrimidin-6-yl)spiro[2.3]hexan-5-ol;
trans-1,1-difluoro-5-(5-(2-methyl-2H-pyrazolo[3,4-b]pyridin-5-yl)[1,3]thiazolo[5,4-b]pyridin-2-yl)spiro[2.3]hexan-5-ol;
cis-1,1-difluoro-5-(5-(2-methyl-2H-pyrazolo[3,4-b]pyridin-5-yl)[1,3]thiazolo[5,4-b]pyridin-2-yl)spiro[2.3]hexan-5-ol;
6,6-difluoro-2-(2-(2-methyl-2H-pyrazolo[3,4-b]pyridin-5-yl)thieno[2,3-d]pyrimidin-6-yl)spiro[3.3]heptan-2-ol;
cis-1-(5-(2-methyl-2H-pyrazolo[3,4-b]pyridin-5-yl)thieno[3,2-b]pyridin-2-yl)-3-(trifluoromethoxy)cyclobutanol;
trans-3-methoxy-3-methyl-1-(2-(2-methyl-2H-pyrazolo[3,4-b]pyridin-5-yl)thieno[2,3-d]pyrimidin-6-yl)cyclobutanol;
cis-3-methoxy-3-methyl-1-(2-(2-methyl-2H-pyrazolo[3,4-b]pyridin-5-yl)thieno[2,3-d]pyrimidin-6-yl)cyclobutanol;
cis-3-(methoxymethyl)-1-(2-(2-methyl-2H-pyrazolo[3,4-b]pyridin-5-yl)thieno[2,3-d]pyrimidin-6-yl)cyclobutanol;
cis-1-(2-(1-methyl-1H-pyrazol-4-yl)thieno[2,3-d]pyrimidin-6-yl)-3-(trifluoromethyl)cyclobutanol;
(S)-(3-amino-6-(2-methyl-2H-pyrazolo[3,4-b]pyridin-5-yl)thieno[2,3-b]pyridin-2-yl)(3,3-difluorocyclobutyl)methanol;
(R)-(3-amino-6-(2-methyl-2H-pyrazolo[3,4-b]pyridin-5-yl)thieno[2,3-b]pyridin-2-yl)(3,3-difluorocyclobutyl)methanol;
trans-3-methoxy-3-methyl-1-(2-(3-methyl-3H-[1,2,3]triazolo[4,5-b]pyridin-6-yl)thieno[2,3-d]pyrimidin-6-yl)cyclobutanol;
cis-3-methoxy-3-methyl-1-(2-(3-methyl-3H-[1,2,3]triazolo[4,5-b]pyridin-6-yl)thieno[2,3-d]pyrimidin-6-yl)cyclobutanol;
cis-1-(2-(3-pyridinyl)thieno[2,3-d]pyrimidin-6-yl)-3-(trifluoromethyl)cyclobutanol;
cis-1-(2-(6-quinoxalinyl)thieno[2,3-d]pyrimidin-6-yl)-3-(trifluoromethyl)cyclobutanol;
cis-1-(2-(imidazo[1,2-a]pyrimidin-6-yl)thieno[2,3-d]pyrimidin-6-yl)-3-(trifluoromethyl)cyclobutanol;
trans-3-methoxy-3-methyl-1-(5-(2-methyl-2H-pyrazolo[3,4-b]pyridin-5-yl)[1,3]thiazolo[5,4-b]pyridin-2-yl)cyclobutanol;
cis-3-methoxy-3-methyl-1-(5-(2-methyl-2H-pyrazolo[3,4-b]pyridin-5-yl)[1,3]thiazolo[5,4-b]pyridin-2-yl)cyclobutanol;
trans-3-methoxy-3-methyl-1-(6-(3-methyl-3H-[1,2,3]triazolo[4,5-b]pyridin-6-yl)thieno[2,3-b]pyridin-2-yl)cyclobutanol;
cis-1-(2-(4-pyridazinyl)thieno[2,3-d]pyrimidin-6-yl)-3-(trifluoromethyl)cyclobutanol;
cis-3-methoxy-3-methyl-1-(5-(2-methyl-2H-pyrazolo[3,4-b]pyridin-5-yl)thieno[3,2-b]pyridin-2-yl)cyclobutanol;
3-(6-(cis-1-hydroxy-3-(trifluoromethyl)cyclobutyl)thieno[2,3-d]pyrimidin-2-yl)-5,6-dihydro-7H-pyrrolo[3,4-b]pyridin-7-one;
cis-1-(2-([1,2,4]triazolo[4,3-a]pyrimidin-6-yl)thieno[2,3-d]pyrimidin-6-yl)-3-(trifluoromethyl)cyclobutanol;
(R)-(3,3-difluorocyclobutyl)(2-(2-methyl-2H-pyrazolo[3,4-b]pyridin-5-yl)thieno[2,3-b]pyrazin-6-yl)methanol;
(S)-(3,3-difluorocyclobutyl)(2-(2-methyl-2H-pyrazolo[3,4-b]pyridin-5-yl)thieno[2,3-b]pyrazin-6-yl)methanol;
trans-3-methyl-1-(6-(2-methyl-2H-pyrazolo[3,4-b]pyridin-5-yl)thieno[2,3-b]pyridin-2-yl)-3-(trifluoromethyl)cyclobutanol;
cis-3-methyl-1-(6-(2-methyl-2H-pyrazolo[3,4-b]pyridin-5-yl)thieno[2,3-b]pyridin-2-yl)-3-(trifluoromethyl)cyclobutanol;
cis-1-(2-(2-methyl-2H-[1,2,3]triazolo[4,5-b]pyridin-6-yl)thieno[2,3-d]pyrimidin-6-yl)-3-(trifluoromethyl)cyclobutanol;
cis-1-(2-(1-methyl-1H-[1,2,3]triazolo[4,5-b]pyridin-6-yl)thieno[2,3-d]pyrimidin-6-yl)-3-(trifluoromethyl)cyclobutanol;
cis-1-(2-(1-methyl-1H-imidazo[4,5-b]pyridin-6-yl)thieno[2,3-d]pyrimidin-6-yl)-3-(trifluoromethyl)cyclobutanol;
cis-1-(2-(1,8-naphthyridin-3-yl)thieno[2,3-d]pyrimidin-6-yl)-3-(trifluoromethyl)cyclobutanol;
trans-3-(difluoromethyl)-1-(5-(2-methyl-2H-pyrazolo[3,4-b]pyridin-5-yl)thiazolo[5,4-b]pyridin-2-yl)cyclobutanol;
trans-3-methoxy-1-(2-(2-methyl-2H-pyrazolo[3,4-b]pyridin-5-yl)thieno[2,3-d]pyrimidin-6-yl)cyclobutanol; or
cis-1-(2-([1,2,4]triazolo[1,5-a]pyrimidin-6-yl)thieno[2,3-d]pyrimidin-6-yl)-3-(trifluoromethyl)cyclobutan-1-ol.
Provided herein as Embodiment 289 is the compound or salt of Embodiment 1, wherein the compound is:
5-(4-cyclobutyl-2-(methoxymethyl)thieno[2,3-b]pyridin-6-yl)-2-methyl-2H-pyrazolo[3,4-b]pyridine;
5-(2-(methoxymethyl)-4-(1-(methoxymethyl)cyclobutyl)thieno[2,3-b]pyridin-6-yl)-2-methyl-2H-pyrazolo[3,4-b]pyridine;
(4,6-bis(1-methyl-1H-pyrazol-5-yl)thieno[2,3-b]pyridin-2-yl)(cyclobutyl)methanol;
cyclobutyl(6-(2-methyl-2H-pyrazolo[3,4-b]pyridin-5-yl)-4-(1-methyl-1H-pyrazol-5-yl)thieno[2,3-b]pyridin-2-yl)methanol;
cyclobutyl(4-(2-methyl-2H-pyrazolo[3,4-b]pyridin-5-yl)-6-(1-methyl-1H-pyrazol-5-yl)thieno[2,3-b]pyridin-2-yl)methanol;
5-(2-(cyclobutyl(methoxy)methyl)-4-(1-methyl-1H-pyrazol-5-yl)thieno[2,3-b]pyridin-6-yl)-2-methyl-2H-pyrazolo[3,4-b]pyridine;
1-cyclobutyl-1-(6-(2-methyl-2H-pyrazolo[3,4-b]pyridin-5-yl)-4-(1-methyl-1H-pyrazol-5-yl)thieno[2,3-b]pyridin-2-yl)ethanol;
1-cyclobutyl-2,2,2-trifluoro-1-(6-(2-methyl-2H-pyrazolo[3,4-b]pyridin-5-yl)-4-(1-methyl-1H-pyrazol-5-yl)thieno[2,3-b]pyridin-2-yl)ethanol;
1-(4-(2-methyl-2H-pyrazolo[3,4-b]pyridin-5-yl)-6-(1-methyl-1H-pyrazol-5-yl)thieno[2,3-b]pyridin-2-yl)ethanol;
1-(6-(2-methyl-2H-pyrazolo[3,4-b]pyridin-5-yl)-4-(1-methyl-1H-pyrazol-5-yl)thieno[2,3-b]pyridin-2-yl)ethanol;
cyclobutyl(6-cyclopropyl-4-(2-methyl-2H-pyrazolo[3,4-b]pyridin-5-yl)thieno[2,3-b]pyridin-2-yl)methanol;
cyclobutyl(4-cyclopropyl-6-(2-methyl-2H-pyrazolo[3,4-b]pyridin-5-yl)thieno[2,3-b]pyridin-2-yl)methanol;
(4-(dimethylamino)-6-(2-methyl-2H-pyrazolo[3,4-b]pyridin-5-yl)thieno[2,3-b]pyridine-2,5-diyl)dimethanol;
1-(4,6-bis(1-methyl-1H-pyrazol-5-yl)thieno[2,3-b]pyridin-2-yl)ethanol;
1-(4,6-bis(2-methyl-2H-pyrazolo[3,4-b]pyridin-5-yl)thieno[2,3-b]pyridin-2-yl)ethanol;
1-(4-cyclopropyl-6-(2-methyl-2H-pyrazolo[3,4-b]pyridin-5-yl)thieno[2,3-b]pyridin-2-yl)ethanol;
1-(6-cyclopropyl-4-(2-methyl-2H-pyrazolo[3,4-b]pyridin-5-yl)thieno[2,3-b]pyridin-2-yl)ethanol;
1-(4-cyclopropyl-6-(2-methyl-2H-pyrazolo[3,4-b]pyridin-5-yl)thieno[2,3-b]pyridin-2-yl)-1-propanol;
(6-(2-methyl-2H-pyrazolo[3,4-b]pyridin-5-yl)thieno[2,3-b]pyridin-2-yl)(tetrahydro-2H-pyran-4-yl)methanol;
cyclopropyl(6-(2-methyl-2H-pyrazolo[3,4-b]pyridin-5-yl)thieno[2,3-b]pyridin-2-yl)methanol;
cyclobutyl(6-(2-methyl-2H-pyrazolo[3,4-b]pyridin-5-yl)thieno[2,3-b]pyridin-2-yl)methanol;
3-(6-(2-methyl-2H-pyrazolo[3,4-b]pyridin-5-yl)thieno[2,3-b]pyridin-2-yl)tetrahydro-3-furanol;
1-(6-(2-methyl-2H-pyrazolo[3,4-b]pyridin-5-yl)thieno[2,3-b]pyridin-2-yl)cyclobutanol;
4-(6-(2-methyl-2H-pyrazolo[3,4-b]pyridin-5-yl)thieno[2,3-b]pyridin-2-yl)tetrahydro-2H-pyran-4-ol;
(6-(2-methyl-2H-pyrazolo[3,4-b]pyridin-5-yl)thieno[2,3-b]pyridin-2-yl)(tetrahydro-3-furanyl)methanol;
3-(6-(2-methyl-2H-pyrazolo[3,4-b]pyridin-5-yl)thieno[2,3-b]pyridin-2-yl)-3-oxetanol;
1-(6-(2-methyl-2H-pyrazolo[3,4-b]pyridin-5-yl)thieno[2,3-b]pyridin-2-yl)cyclopentanol;
1-(6-(2-methyl-2H-pyrazolo[3,4-b]pyridin-5-yl)thieno[2,3-b]pyridin-2-yl)-1-propanol;
(6-(2-methyl-2H-pyrazolo[3,4-b]pyridin-5-yl)thieno[2,3-b]pyridin-2-yl)methanol;
2,2,2-trifluoro-1-(6-(2-methyl-2H-pyrazolo[3,4-b]pyridin-5-yl)thieno[2,3-b]pyridin-2-yl)ethanol;
2-methyl-1-(6-(2-methyl-2H-pyrazolo[3,4-b]pyridin-5-yl)thieno[2,3-b]pyridin-2-yl)-1-propanol;
1-(6-(2-methyl-2H-pyrazolo[3,4-b]pyridin-5-yl)thieno[2,3-b]pyridin-2-yl)ethanol;
1-(6-(2-methyl-2H-pyrazolo[3,4-b]pyridin-5-yl)thieno[2,3-b]pyridin-2-yl)-1-butanol;
(6-(2-methyl-2H-pyrazolo[3,4-b]pyridin-5-yl)thieno[2,3-b]pyridin-2-yl)(3-oxetanyl)methanol;
3-methoxy-1-(6-(2-methyl-2H-pyrazolo[3,4-b]pyridin-5-yl)thieno[2,3-b]pyridin-2-yl)-1-propanol;
4,4-difluoro-1-(6-(2-methyl-2H-pyrazolo[3,4-b]pyridin-5-yl)thieno[2,3-b]pyridin-2-yl)cyclohexanol;
1,1,1-trifluoro-3-methoxy-2-(6-(2-methyl-2H-pyrazolo[3,4-b]pyridin-5-yl)thieno[2,3-b]pyridin-2-yl)-2-propanol;
1-cyclopropyl-2,2,2-trifluoro-1-(6-(2-methyl-2H-pyrazolo[3,4-b]pyridin-5-yl)thieno[2,3-b]pyridin-2-yl)ethanol;
(3,3-difluorocyclobutyl)(6-(2-methyl-2H-pyrazolo[3,4-b]pyridin-5-yl)thieno[2,3-b]pyridin-2-yl)methanol;
(4,4-difluorocyclohexyl)(6-(2-methyl-2H-pyrazolo[3,4-b]pyridin-5-yl)thieno[2,3-b]pyridin-2-yl)methanol;
1-methyl-3-(6-(2-methyl-2H-pyrazolo[3,4-b]pyridin-5-yl)thieno[2,3-b]pyridin-2-yl)-3-azetidinol;
1-methyl-3-(6-(2-methyl-2H-pyrazolo[3,4-b]pyridin-5-yl)thieno[2,3-b]pyridin-2-yl)-3-piperidinol;
1,1,1-trifluoro-2-(6-(2-methyl-2H-pyrazolo[3,4-b]pyridin-5-yl)thieno[2,3-b]pyridin-2-yl)-2-butanol;
2-methoxy-1-(6-(2-methyl-2H-pyrazolo[3,4-b]pyridin-5-yl)thieno[2,3-b]pyridin-2-yl)ethanol;
4-(6-(2-methyl-2H-pyrazolo[3,4-b]pyridin-5-yl)thieno[2,3-b]pyridin-2-yl)tetrahydro-2H-thiopyran-4-ol 1,1-dioxide;
2-(6-(2-methyl-2H-pyrazolo[3,4-b]pyridin-5-yl)thieno[2,3-b]pyridin-2-yl)-2-propanol;
2-(6-(2-methyl-2H-pyrazolo[3,4-b]pyridin-5-yl)thieno[2,3-b]pyridin-2-yl)-2-butanol;
1-cyclopropyl-1-(6-(2-methyl-2H-pyrazolo[3,4-b]pyridin-5-yl)thieno[2,3-b]pyridin-2-yl)ethanol;
1-methyl-4-(6-(2-methyl-2H-pyrazolo[3,4-b]pyridin-5-yl)thieno[2,3-b]pyridin-2-yl)-4-piperidinol;
1-methoxy-2-(6-(2-methyl-2H-pyrazolo[3,4-b]pyridin-5-yl)thieno[2,3-b]pyridin-2-yl)-2-propanol;
3,3-difluoro-1-(6-(2-methyl-2H-pyrazolo[3,4-b]pyridin-5-yl)thieno[2,3-b]pyridin-2-yl)cyclobutanol;
1-methyl-3-(6-(2-methyl-2H-pyrazolo[3,4-b]pyridin-5-yl)thieno[2,3-b]pyridin-2-yl)-3-pyrrolidinol;
(1-methyl-4-piperidinyl)(6-(2-methyl-2H-pyrazolo[3,4-b]pyridin-5-yl)thieno[2,3-b]pyridin-2-yl)methanol;
(6-(1-methyl-1H-indazol-5-yl)thieno[2,3-b]pyridin-2-yl)(tetrahydro-2H-pyran-4-yl)methanol;
(6-(1-methyl-1H-pyrazolo[3,4-b]pyridin-5-yl)thieno[2,3-b]pyridin-2-yl)(tetrahydro-2H-pyran-4-yl)methanol;
1,1,1-trifluoro-4-methoxy-2-(6-(2-methyl-2H-pyrazolo[3,4-b]pyridin-5-yl)thieno[2,3-b]pyridin-2-yl)-2-butanol;
(6-(1H-indazol-5-yl)thieno[2,3-b]pyridin-2-yl)(tetrahydro-2H-pyran-4-yl)methanol;
(6-(6-methyl-1H-indazol-5-yl)thieno[2,3-b]pyridin-2-yl)(tetrahydro-2H-pyran-4-yl)methanol;
1,1,1-trifluoro-2-(6-(2-methyl-2H-pyrazolo[3,4-b]pyridin-5-yl)thieno[2,3-b]pyridin-2-yl)-2-propanol;
(6-(2-methyl-2H-[1,2,3]triazolo[4,5-b]pyridin-6-yl)thieno[2,3-b]pyridin-2-yl)(tetrahydro-2H-pyran-4-yl)methanol;
(6-(3-methyl-1H-indazol-5-yl)thieno[2,3-b]pyridin-2-yl)(tetrahydro-2H-pyran-4-yl)methanol;
4-methoxy-2-(6-(2-methyl-2H-pyrazolo[3,4-b]pyridin-5-yl)thieno[2,3-b]pyridin-2-yl)-2-butanol;
(6-(3-methyl-3H-[1,2,3]triazolo[4,5-b]pyridin-6-yl)thieno[2,3-b]pyridin-2-yl)(tetrahydro-2H-pyran-4-yl)methanol;
(1-methyl-3-azetidinyl)(6-(2-methyl-2H-pyrazolo[3,4-b]pyridin-5-yl)thieno[2,3-b]pyridin-2-yl)methanol;
(6-(1,2-benzoxazol-5-yl)thieno[2,3-b]pyridin-2-yl)(tetrahydro-2H-pyran-4-yl)methanol;
tetrahydro-2H-pyran-4-yl(6-(3H-[1,2,3]triazolo[4,5-b]pyridin-6-yl)thieno[2,3-b]pyridin-2-yl)methanol;
(1-methyl-2-azetidinyl)(6-(2-methyl-2H-pyrazolo[3,4-b]pyridin-5-yl)thieno[2,3-b]pyridin-2-yl)methanol;
(3,3-difluorocyclobutyl)(6-(2-methyl-1,3-benzoxazol-5-yl)thieno[2,3-b]pyridin-2-yl)methanol;
(3,3-difluorocyclobutyl)(6-(1H-pyrazolo[3,4-b]pyridin-5-yl)thieno[2,3-b]pyridin-2-yl)methanol;
(3,3-difluorocyclobutyl)(6-(2-methylimidazo[1,2-a]pyridin-6-yl)thieno[2,3-b]pyridin-2-yl)methanol;
6-(2-((3,3-difluorocyclobutyl)(hydroxy)methyl)thieno[2,3-b]pyridin-6-yl)-3-methyl-4(3H)-pyrimidinone;
6-(2-((3,3-difluorocyclobutyl)(hydroxy)methyl)thieno[2,3-b]pyridin-6-yl)-2-methyl-1(2H)-isoquinolinone;
(3,3-difluorocyclobutyl)(6-(2-methyl-1,3-benzoxazol-6-yl)thieno[2,3-b]pyridin-2-yl)methanol;
(3,3-difluorocyclobutyl)(6-(6-quinoxalinyl)thieno[2,3-b]pyridin-2-yl)methanol;
(3,3-difluorocyclobutyl)(6-(2-ethyl-2H-pyrazolo[3,4-b]pyridin-5-yl)thieno[2,3-b]pyridin-2-yl)methanol;
1-(6-(2-methyl-2H-pyrazolo[3,4-b]pyridin-5-yl)-1-benzothiophen-2-yl)-1-butanol;
(3,3-difluorocyclobutyl)(6-(2-methylimidazo[1,2-a]pyridin-7-yl)thieno[2,3-b]pyridin-2-yl)methanol;
(3,3-difluorocyclobutyl)(6-(2-(2-propanyl)-2H-indazol-5-yl)thieno[2,3-b]pyridin-2-yl)methanol;
(3,3-difluorocyclobutyl)(6-(5-pyrimidinyl)thieno[2,3-b]pyridin-2-yl)methanol;
cyclopentyl(6-(2-methyl-2H-pyrazolo[3,4-b]pyridin-5-yl)-1-benzothiophen-2-yl)methanol;
3-methoxy-1-(6-(2-methyl-2H-pyrazolo[3,4-b]pyridin-5-yl)-1-benzothiophen-2-yl)-1-propanol;
4,4,4-trifluoro-1-(6-(2-methyl-2H-pyrazolo[3,4-b]pyridin-5-yl)-1-benzothiophen-2-yl)-1-butanol;
(3,3-difluorocyclobutyl)(6-(2-methyl-2H-pyrazolo[3,4-b]pyridin-5-yl)-1-benzothiophen-2-yl)methanol;
(6-(2-methyl-2H-pyrazolo[3,4-b]pyridin-5-yl)thieno[2,3-b]pyridin-2-yl)(1-methyl-3-pyrrolidinyl)methanol;
(1-methyl-3-piperidinyl)(6-(2-methyl-2H-pyrazolo[3,4-b]pyridin-5-yl)thieno[2,3-b]pyridin-2-yl)methanol;
(3,3-difluorocyclobutyl)(6-(2-(2-propanyl)-2H-pyrazolo[3,4-b]pyridin-5-yl)thieno[2,3-b]pyridin-2-yl)methanol;
(3,3-difluorocyclobutyl)(6-(1-(2-propanyl)-1H-pyrazolo[3,4-b]pyridin-5-yl)thieno[2,3-b]pyridin-2-yl)methanol;
(6-(2-methyl-2H-pyrazolo[3,4-b]pyridin-5-yl)thieno[2,3-b]pyridin-2-yl)(tetrahydro-2H-pyran-4-yl)methan-d-ol;
(3-amino-6-(5-pyrimidinyl)thieno[2,3-b]pyridin-2-yl)(3,3-difluorocyclobutyl)methanol;
2-(6-(2-methyl-2H-pyrazolo[3,4-b]pyridin-5-yl)thieno[2,3-b]pyridin-2-yl)spiro[3.3]heptan-2-ol;
3,3-dimethyl-1-(6-(2-methyl-2H-pyrazolo[3,4-b]pyridin-5-yl)thieno[2,3-b]pyridin-2-yl)cyclobutanol;
6,6-difluoro-2-(6-(2-methyl-2H-pyrazolo[3,4-b]pyridin-5-yl)thieno[2,3-b]pyridin-2-yl)spiro[3.3]heptan-2-ol;
6-(6-(2-methyl-2H-pyrazolo[3,4-b]pyridin-5-yl)thieno[2,3-b]pyridin-2-yl)-2-oxaspiro[3.3]heptan-6-ol;
1-(6-(2-methyl-2H-pyrazolo[3,4-b]pyridin-5-yl)thieno[2,3-b]pyridin-2-yl)-3-(trifluoromethyl)cyclobutanol;
3-(difluoromethyl)-1-(6-(2-methyl-2H-pyrazolo[3,4-b]pyridin-5-yl)thieno[2,3-b]pyridin-2-yl)cyclobutanol;
3-methoxy-1-(6-(2-methyl-2H-pyrazolo[3,4-b]pyridin-5-yl)thieno[2,3-b]pyridin-2-yl)cyclobutanol;
(3,3-difluorocyclobutyl)(5-(2-methyl-2H-pyrazolo[3,4-b]pyridin-5-yl)[1,3]thiazolo[5,4-b]pyridin-2-yl)methanol;
1-(6-(2-ethyl-2H-pyrazolo[3,4-b]pyridin-5-yl)thieno[2,3-b]pyridin-2-yl)-3,3-difluorocyclobutanol;
(3,3-difluorocyclobutyl)(5-(2-methyl-2H-pyrazolo[3,4-b]pyridin-5-yl)thieno[3,2-b]pyridin-2-yl)methanol;
3,3-difluoro-1-(6-(3-methyl-3H-imidazo[4,5-b]pyridin-6-yl)thieno[2,3-b]pyridin-2-yl)cyclobutanol;
3,3-difluoro-1-(6-(2-methyl[1,3]oxazolo[4,5-b]pyridin-6-yl)thieno[2,3-b]pyridin-2-yl)cyclobutanol;
3,3-difluoro-1-(6-(2-methyl-3H-imidazo[4,5-b]pyridin-6-yl)thieno[2,3-b]pyridin-2-yl)cyclobutanol;
3,3-difluoro-1-(6-(1H-pyrazolo[3,4-b]pyridin-5-yl)thieno[2,3-b]pyridin-2-yl)cyclobutanol;
3,3-difluoro-1-(6-(2-methylimidazo[1,2-a]pyridin-6-yl)thieno[2,3-b]pyridin-2-yl)cyclobutanol;
1-(6-(2-amino-5-pyrimidinyl)thieno[2,3-b]pyridin-2-yl)-3,3-difluorocyclobutanol;
(3,3-difluorocyclobutyl)(5-(2-methyl-2H-pyrazolo[3,4-b]pyridin-5-yl)[1,3]thiazolo[4,5-b]pyridin-2-yl)methanol;
3,3-difluoro-1-(6-(2-methyl-1,3-benzoxazol-6-yl)thieno[2,3-b]pyridin-2-yl)cyclobutanol;
3-fluoro-1-(6-(2-methyl-2H-pyrazolo[3,4-b]pyridin-5-yl)thieno[2,3-b]pyridin-2-yl)cyclobutanol;
(3,3-difluorocyclobutyl)(2-(2-methyl-2H-pyrazolo[3,4-b]pyridin-5-yl)thieno[2,3-d]pyrimidin-6-yl)methanol;
(3-chloro-6-(2-methyl-2H-pyrazolo[3,4-b]pyridin-5-yl)thieno[2,3-b]pyridin-2-yl)(3,3-difluorocyclobutyl)methanol;
3,3-difluoro-1-(6-(3H-imidazo[4,5-b]pyridin-6-yl)thieno[2,3-b]pyridin-2-yl)cyclobutanol;
3,3-difluoro-1-(6-(1H-indazol-5-yl)thieno[2,3-b]pyridin-2-yl)cyclobutanol;
(3,3-difluorocyclobutyl)(5-(2-methyl-2H-pyrazolo[3,4-b]pyridin-5-yl)thieno[2,3-b]pyridin-2-yl)methanol;
(3,3-difluorocyclobutyl)(6-(2-methyl-2H-pyrazolo[3,4-b]pyridin-5-yl)thieno[3,2-b]pyridin-2-yl)methanol;
3,3-difluoro-1-(6-(2-(methylamino)-5-pyrimidinyl)thieno[2,3-b]pyridin-2-yl)cyclobutanol;
(3,3-difluorocyclobutyl)(6-(2-methyl-2H-pyrazolo[3,4-b]pyridin-5-yl)thieno[2,3-b]pyridin-2-yl)methan-d-ol;
3,3-difluoro-1-(6-(2-methylimidazo[1,2-a]pyridin-7-yl)thieno[2,3-b]pyridin-2-yl)cyclobutanol;
3,3-difluoro-1-(6-(2-methylimidazo[1,2-a]pyrimidin-6-yl)thieno[2,3-b]pyridin-2-yl)cyclobutanol;
2-(3,3-difluorocyclobutyl)-2-(6-(2-methyl-2H-pyrazolo[3,4-b]pyridin-5-yl)thieno[2,3-b]pyridin-2-yl)ethanol;
3,3-difluoro-1-(6-(5-pyrimidinyl)thieno[2,3-b]pyridin-2-yl)cyclobutanol;
3,3-difluoro-1-(6-(pyrido[2,3-b]pyrazin-7-yl)thieno[2,3-b]pyridin-2-yl)cyclobutanol;
(3,3-difluorocyclobutyl)(2-(2-methyl-2H-pyrazolo[3,4-b]pyridin-5-yl)-7-quinolinyl)methanol;
3,3-difluoro-1-(6-(2-methyl-2H-pyrazolo[3,4-b]pyridin-5-yl)-1-benzothiophen-2-yl)cyclobutanol;
3,3-difluoro-1-(6-(2-methyl-2H-indazol-5-yl)thieno[2,3-b]pyridin-2-yl)cyclobutanol;
1-(6-(1,2-benzoxazol-5-yl)thieno[2,3-b]pyridin-2-yl)-3,3-difluorocyclobutanol;
3,3-difluoro-1-(6-(3-methyl-3H-[1,2,3]triazolo[4,5-b]pyridin-6-yl)thieno[2,3-b]pyridin-2-yl)cyclobutanol;
2-(6-(2-methyl-2H-pyrazolo[3,4-b]pyridin-5-yl)thieno[2,3-b]pyridin-2-yl)spiro[3.5]nonan-2-ol;
2-(6-(2-methyl-2H-pyrazolo[3,4-b]pyridin-5-yl)thieno[2,3-b]pyridin-2-yl)spiro[3.4]octan-2-ol;
3,3-difluoro-1-(6-(2-methylimidazo[1,2-b]pyridazin-7-yl)thieno[2,3-b]pyridin-2-yl)cyclobutanol;
3,3-difluoro-1-(6-(3H-[1,2,3]triazolo[4,5-b]pyridin-6-yl)thieno[2,3-b]pyridin-2-yl)cyclobutanol;
(3,3-difluorocyclobutyl)(6-(3-methyl-3H-[1,2,3]triazolo[4,5-b]pyridin-6-yl)thieno[2,3-b]pyridin-2-yl)methanol;
6-(6-(2-methyl-2H-pyrazolo[3,4-b]pyridin-5-yl)thieno[2,3-b]pyridin-2-yl)-1-oxaspiro[3.3]heptan-6-ol;
3,3-dimethyl-1-(2-(2-methyl-2H-pyrazolo[3,4-b]pyridin-5-yl)thieno[2,3-d]pyrimidin-6-yl)cyclobutanol;
3-(methoxymethyl)-1-(6-(2-methyl-2H-pyrazolo[3,4-b]pyridin-5-yl)thieno[2,3-b]pyridin-2-yl)cyclobutanol;
3-methoxy-3-methyl-1-(6-(2-methyl-2H-pyrazolo[3,4-b]pyridin-5-yl)thieno[2,3-b]pyridin-2-yl)cyclobutanol;
(3,3-difluorocyclobutyl)(3-methyl-6-(2-methyl-2H-pyrazolo[3,4-b]pyridin-5-yl)thieno[2,3-b]pyridin-2-yl)methanol;
1-(6-(2-methyl-2H-pyrazolo[3,4-b]pyridin-5-yl)thieno[2,3-b]pyridin-2-yl)cyclopropanol;
5-(6-(2-methyl-2H-pyrazolo[3,4-b]pyridin-5-yl)thieno[2,3-b]pyridin-2-yl)spiro[2.3]hexan-5-ol;
3-cyclopropyl-1-(6-(2-methyl-2H-pyrazolo[3,4-b]pyridin-5-yl)thieno[2,3-b]pyridin-2-yl)cyclobutanol;
3-hydroxy-1-methyl-3-(6-(2-methyl-2H-pyrazolo[3,4-b]pyridin-5-yl)thieno[2,3-b]pyridin-2-yl)cyclobutanecarbonitrile;
2-methyl-1-(6-(2-methyl-2H-pyrazolo[3,4-b]pyridin-5-yl)thieno[2,3-b]pyridin-2-yl)cyclobutanol;
2,2-dimethyl-1-(6-(2-methyl-2H-pyrazolo[3,4-b]pyridin-5-yl)thieno[2,3-b]pyridin-2-yl)cyclobutanol;
3-methoxy-1-(6-(2-methyl-2H-pyrazolo[3,4-b]pyridin-5-yl)thieno[2,3-b]pyridin-2-yl)-3-(trifluoromethyl)cyclobutanol;
1-(6-(2-methyl-2H-pyrazolo[3,4-b]pyridin-5-yl)thieno[2,3-b]pyridin-2-yl)-3-(trifluoromethoxy)cyclobutanol;
3,3-dimethyl-1-(5-(2-methyl-2H-pyrazolo[3,4-b]pyridin-5-yl)[1,3]thiazolo[5,4-b]pyridin-2-yl)cyclobutanol;
1-(3-chloro-6-(2-methyl-2H-pyrazolo[3,4-b]pyridin-5-yl)thieno[2,3-b]pyridin-2-yl)-3-methoxycyclobutanol;
2-methoxy-1-(6-(2-methyl-2H-pyrazolo[3,4-b]pyridin-5-yl)thieno[2,3-b]pyridin-2-yl)cyclobutanol;
1-(2-(2-methyl-2H-pyrazolo[3,4-b]pyridin-5-yl)thieno[2,3-d]pyrimidin-6-yl)-3-(trifluoromethyl)cyclobutanol;
1-(3-chloro-6-(2-methyl-2H-pyrazolo[3,4-b]pyridin-5-yl)thieno[2,3-b]pyridin-2-yl)-3,3-dimethylcyclobutanol;
1-(5-(2-methyl-2H-pyrazolo[3,4-b]pyridin-5-yl)[1,3]thiazolo[5,4-b]pyridin-2-yl)-3-(trifluoromethyl)cyclobutanol;
1-(6-(2-methyl-2H-pyrazolo[3,4-b]pyridin-5-yl)thieno[2,3-b]pyridin-2-yl)-3-(trifluoromethoxy)cyclobutan-1-ol;
(6-(2-methyl-2H-pyrazolo[3,4-b]pyridin-5-yl)thieno[2,3-b]pyridin-2-yl)(3-(trifluoromethyl)cyclobutyl)methanol;
1-(3-chloro-6-(2-methyl-2H-pyrazolo[3,4-b]pyridin-5-yl)thieno[2,3-b]pyridin-2-yl)-3-(trifluoromethyl)cyclobutanol;
(2,2-difluorocyclopropyl)(6-(2-methyl-2H-pyrazolo[3,4-b]pyridin-5-yl)thieno[2,3-b]pyridin-2-yl)methanol;
(3,3-difluoro-1-methylcyclobutyl)(6-(2-methyl-2H-pyrazolo[3,4-b]pyridin-5-yl)thieno[2,3-b]pyridin-2-yl)methanol;
bicyclo [1.1.1]pentan-1-yl(6-(2-methyl-2H-pyrazolo[3,4-b]pyridin-5-yl)thieno[2,3-b]pyridin-2-yl)methanol;
(3-methoxycyclobutyl)(6-(2-methyl-2H-pyrazolo[3,4-b]pyridin-5-yl)thieno[2,3-b]pyridin-2-yl)methanol;
3-(difluoromethyl)-1-(6-(3-methyl-3H-[1,2,3]triazolo[4,5-b]pyridin-6-yl)thieno[2,3-b]pyridin-2-yl)cyclobutanol;
3,3-dimethyl-1-(2-(2-methyl-2H-pyrazolo[3,4-b]pyridin-5-yl)-7-quinolinyl)cyclobutanol;
3,3-difluoro-1-(3-methyl-6-(2-methyl-2H-pyrazolo[3,4-b]pyridin-5-yl)thieno[2,3-b]pyridin-2-yl)cyclobutanol;
1-(3-methyl-6-(2-methyl-2H-pyrazolo[3,4-b]pyridin-5-yl)thieno[2,3-b]pyridin-2-yl)-3-(trifluoromethyl)cyclobutanol;
1-(6-(3-methyl-3H-[1,2,3]triazolo[4,5-b]pyridin-6-yl)thieno[2,3-b]pyridin-2-yl)-3-(trifluoromethyl)cyclobutanol;
3-(difluoromethyl)-1-(2-(2-methyl-2H-pyrazolo[3,4-b]pyridin-5-yl)thieno[2,3-d]pyrimidin-6-yl)cyclobutanol;
(2-(3-methyl-3H-[1,2,3]triazolo[4,5-b]pyridin-6-yl)-7-quinolinyl)(tetrahydro-2H-pyran-4-yl)methanol;
(2-(2-methyl-2H-pyrazolo[3,4-b]pyridin-5-yl)-7-quinolinyl)(tetrahydro-2H-pyran-4-yl)methanol;
3-(difluoromethyl)-1-(5-(2-methyl-2H-pyrazolo[3,4-b]pyridin-5-yl)[1,3]thiazolo[5,4-b]pyridin-2-yl)cyclobutanol;
3-(difluoromethyl)-1-(5-(3-methyl-3H-[1,2,3]triazolo[4,5-b]pyridin-6-yl)[1,3]thiazolo[5,4-b]pyridin-2-yl)cyclobutanol;
(3-methyl-6-(3-methyl-3H-[1,2,3]triazolo[4,5-b]pyridin-6-yl)thieno[2,3-b]pyridin-2-yl)(tetrahydro-2H-pyran-4-yl)methanol;
(3-methyl-6-(2-methyl-2H-pyrazolo[3,4-b]pyridin-5-yl)thieno[2,3-b]pyridin-2-yl)(tetrahydro-2H-pyran-4-yl)methanol;
3-(difluoromethyl)-1-(2-(2-methyl-2H-pyrazolo[3,4-b]pyridin-5-yl)-7-quinolinyl)cyclobutanol;
(6-(2-methyl-2H-pyrazolo[3,4-b]pyridin-5-yl)thieno[2,3-b]pyridin-2-yl)(3-(trifluoromethyl)bicyclo[1.1.1]pentan-1-yl)methanol;
3-(difluoromethyl)-1-(3-methyl-6-(2-methyl-2H-pyrazolo[3,4-b]pyridin-5-yl)thieno[2,3-b]pyridin-2-yl)cyclobutanol;
3-(difluoromethyl)-1-(2-(2-methyl-2H-pyrazolo[3,4-b]pyridin-5-yl)-6-quinolinyl)cyclobutanol;
3-(difluoromethyl)-1-(5-(2-methyl-2H-pyrazolo[3,4-b]pyridin-5-yl)thieno[3,2-b]pyridin-2-yl)cyclobutanol;
3-(difluoromethyl)-1-(2-(3-methyl-3H-[1,2,3]triazolo[4,5-b]pyridin-6-yl)thieno[2,3-d]pyrimidin-6-yl)cyclobutanol;
3-fluoro-3-(6-(2-methyl-2H-pyrazolo[3,4-b]pyridin-5-yl)thieno[2,3-b]pyridin-2-yl)cyclobutanol;
1-(5-(2-methyl-2H-pyrazolo[3,4-b]pyridin-5-yl)thieno[3,2-b]pyridin-2-yl)-3-(trifluoromethyl)cyclobutanol;
(3,3-difluorocyclobutyl)(2-(2-methyl-2H-pyrazolo[3,4-b]pyridin-5-yl)-6-quinolinyl)methanol;
3-(difluoromethyl)-1-(6-(2-ethyl-2H-pyrazolo[3,4-b]pyridin-5-yl)thieno[2,3-b]pyridin-2-yl)cyclobutanol;
1,1-difluoro-5-(6-(2-methyl-2H-pyrazolo[3,4-b]pyridin-5-yl)thieno[2,3-b]pyridin-2-yl)spiro[2.3]hexan-5-ol;
3-(difluoromethyl)-1-(2-(2-methyl-2H-pyrazolo[3,4-b]pyridin-5-yl)thieno[3,2-d]pyrimidin-6-yl)cyclobutanol;
3-(difluoromethyl)-1-(5-methyl-2-(2-methyl-2H-pyrazolo[3,4-b]pyridin-5-yl)thieno[2,3-d]pyrimidin-6-yl)cyclobutanol;
3-hydroxy-1-methyl-3-(5-(2-methyl-2H-pyrazolo[3,4-b]pyridin-5-yl)[1,3]thiazolo[5,4-b]pyridin-2-yl)cyclobutanecarbonitrile;
3-(difluoromethyl)-1-(6-(2-methylimidazo[1,2-b]pyridazin-7-yl)thieno[2,3-b]pyridin-2-yl)cyclobutanol;
3-(difluoromethyl)-1-(6-(2-methyl-3H-imidazo[4,5-b]pyridin-6-yl)thieno[2,3-b]pyridin-2-yl)cyclobutanol;
(3,3-difluorocyclobutyl)(3-(2-methyl-2H-pyrazolo[3,4-b]pyridin-5-yl)thieno[2,3-b]pyrazin-6-yl)methanol;
2-(3,3-difluorocyclobutyl)-2-(3-methyl-6-(2-methyl-2H-pyrazolo[3,4-b]pyridin-5-yl)thieno[2,3-b]pyridin-2-yl)ethanol;
3-methoxy-1-(2-(2-methyl-2H-pyrazolo[3,4-b]pyridin-5-yl)thieno[2,3-d]pyrimidin-6-yl)cyclobutanol;
1-(2-(3-methyl-3H-[1,2,3]triazolo[4,5-b]pyridin-6-yl)thieno[2,3-d]pyrimidin-6-yl)-3-(trifluoromethyl)cyclobutanol;
3-hydroxy-1-methyl-3-(2-(2-methyl-2H-pyrazolo[3,4-b]pyridin-5-yl)thieno[2,3-d]pyrimidin-6-yl)cyclobutanecarbonitrile;
3-(difluoromethyl)-1-(6-(2-methylimidazo[1,2-a]pyrimidin-6-yl)thieno[2,3-b]pyridin-2-yl)cyclobutanol;
3-hydroxy-1-methyl-3-(2-(3-methyl-3H-[1,2,3]triazolo[4,5-b]pyridin-6-yl)thieno[2,3-d]pyrimidin-6-yl)cyclobutanecarbonitrile;
3-cyclopropyl-1-(2-(2-methyl-2H-pyrazolo[3,4-b]pyridin-5-yl)thieno[2,3-d]pyrimidin-6-yl)cyclobutanol;
1-(2-(2-methyl-2H-pyrazolo[3,4-b]pyridin-5-yl)thieno[2,3-d]pyrimidin-6-yl)-3-(trifluoromethoxy)cyclobutanol;
1-(6-(3-methyl-3H-[1,2,3]triazolo[4,5-b]pyridin-6-yl)thieno[2,3-b]pyridin-2-yl)-3-(trifluoromethoxy)cyclobutanol;
3-methoxy-3-methyl-1-(6-(3-methyl-3H-[1,2,3]triazolo[4,5-b]pyridin-6-yl)thieno[2,3-b]pyridin-2-yl)cyclobutanol;
3-(difluoromethyl)-1-(6-(3H-[1,2,3]triazolo[4,5-b]pyridin-6-yl)thieno[2,3-b]pyridin-2-yl)cyclobutanol;
3-hydroxy-1-methyl-3-(6-(3-methyl-3H-[1,2,3]triazolo[4,5-b]pyridin-6-yl)thieno[2,3-b]pyridin-2-yl)cyclobutanecarbonitrile;
1-(5-(2-methyl-2H-pyrazolo[3,4-b]pyridin-5-yl)[1,3]thiazolo[5,4-b]pyridin-2-yl)-3-(trifluoromethoxy)cyclobutanol;
3-hydroxy-1-methyl-3-(5-(2-methyl-2H-pyrazolo[3,4-b]pyridin-5-yl)thieno[3,2-b]pyridin-2-yl)cyclobutanecarbonitrile;
5-(2-(2-methyl-2H-pyrazolo[3,4-b]pyridin-5-yl)thieno[2,3-d]pyrimidin-6-yl)spiro[2.3]hexan-5-ol;
5-(5-(2-methyl-2H-pyrazolo[3,4-b]pyridin-5-yl)[1,3]thiazolo[5,4-b]pyridin-2-yl)spiro[2.3]hexan-5-ol;
2,2-difluorocyclobutyl)(6-(2-methyl-2H-pyrazolo[3,4-b]pyridin-5-yl)thieno[2,3-b]pyridin-2-yl)methanol;
3-(difluoromethyl)-1-(5-(2-methyl-2H-pyrazolo[3,4-b]pyridin-5-yl)[1,3]thiazolo[5,4-d]pyrimidin-2-yl)cyclobutanol;
1-(2-(3-methyl-3H-[1,2,3]triazolo[4,5-b]pyridin-6-yl)thieno[2,3-d]pyrimidin-6-yl)-3-(trifluoromethoxy)cyclobutanol;
1-(5-(3-methyl-3H-[1,2,3]triazolo[4,5-b]pyridin-6-yl)[1,3]thiazolo[5,4-b]pyridin-2-yl)-3-(trifluoromethoxy)cyclobutanol;
3-methoxy-1-(2-(2-methyl-2H-pyrazolo[3,4-b]pyridin-5-yl)thieno[2,3-d]pyrimidin-6-yl)-3-(trifluoromethyl)cyclobutanol;
1,1-difluoro-5-(2-(2-methyl-2H-pyrazolo[3,4-b]pyridin-5-yl)thieno[2,3-d]pyrimidin-6-yl)spiro[2.3]hexan-5-ol;
1,1-difluoro-5-(5-(2-methyl-2H-pyrazolo[3,4-b]pyridin-5-yl)[1,3]thiazolo[5,4-b]pyridin-2-yl)spiro[2.3]hexan-5-ol;
6,6-difluoro-2-(2-(2-methyl-2H-pyrazolo[3,4-b]pyridin-5-yl)thieno[2,3-d]pyrimidin-6-yl)spiro[3.3]heptan-2-ol;
1-(5-(2-methyl-2H-pyrazolo[3,4-b]pyridin-5-yl)thieno[3,2-b]pyridin-2-yl)-3-(trifluoromethoxy)cyclobutanol;
3-methoxy-3-methyl-1-(2-(2-methyl-2H-pyrazolo[3,4-b]pyridin-5-yl)thieno[2,3-d]pyrimidin-6-yl)cyclobutanol;
3-(methoxymethyl)-1-(2-(2-methyl-2H-pyrazolo[3,4-b]pyridin-5-yl)thieno[2,3-d]pyrimidin-6-yl)cyclobutanol;
1-(2-(1-methyl-1H-pyrazol-4-yl)thieno[2,3-d]pyrimidin-6-yl)-3-(trifluoromethyl)cyclobutanol;
(3-amino-6-(2-methyl-2H-pyrazolo[3,4-b]pyridin-5-yl)thieno[2,3-b]pyridin-2-yl)(3,3-difluorocyclobutyl)methanol;
3-methoxy-3-methyl-1-(2-(3-methyl-3H-[1,2,3]triazolo[4,5-b]pyridin-6-yl)thieno[2,3-d]pyrimidin-6-yl)cyclobutanol;
1-(2-(3-pyridinyl)thieno[2,3-d]pyrimidin-6-yl)-3-(trifluoromethyl)cyclobutanol;
1-(2-(6-quinoxalinyl)thieno[2,3-d]pyrimidin-6-yl)-3-(trifluoromethyl)cyclobutanol;
1-(2-(imidazo[1,2-a]pyrimidin-6-yl)thieno[2,3-d]pyrimidin-6-yl)-3-(trifluoromethyl)cyclobutanol;
3-methoxy-3-methyl-1-(5-(2-methyl-2H-pyrazolo[3,4-b]pyridin-5-yl)[1,3]thiazolo[5,4-b]pyridin-2-yl)cyclobutanol;
1-(2-(4-pyridazinyl)thieno[2,3-d]pyrimidin-6-yl)-3-(trifluoromethyl)cyclobutanol;
3-methoxy-3-methyl-1-(5-(2-methyl-2H-pyrazolo[3,4-b]pyridin-5-yl)thieno[3,2-b]pyridin-2-yl)cyclobutanol;
3-(6-(1-hydroxy-3-(trifluoromethyl)cyclobutyl)thieno[2,3-d]pyrimidin-2-yl)-5,6-dihydro-7H-pyrrolo[3,4-b]pyridin-7-one;
1-(2-([1,2,4]triazolo[4,3-a]pyrimidin-6-yl)thieno[2,3-d]pyrimidin-6-yl)-3-(trifluoromethyl)cyclobutanol;
(3,3-difluorocyclobutyl)(2-(2-methyl-2H-pyrazolo[3,4-b]pyridin-5-yl)thieno[2,3-b]pyrazin-6-yl)methanol;
3-methyl-1-(6-(2-methyl-2H-pyrazolo[3,4-b]pyridin-5-yl)thieno[2,3-b]pyridin-2-yl)-3-(trifluoromethyl)cyclobutanol;
1-(2-(2-methyl-2H-[1,2,3]triazolo[4,5-b]pyridin-6-yl)thieno[2,3-d]pyrimidin-6-yl)-3-(trifluoromethyl)cyclobutanol;
1-(2-(1-methyl-1H-[1,2,3]triazolo[4,5-b]pyridin-6-yl)thieno[2,3-d]pyrimidin-6-yl)-3-(trifluoromethyl)cyclobutanol;
1-(2-(1-methyl-1H-imidazo[4,5-b]pyridin-6-yl)thieno[2,3-d]pyrimidin-6-yl)-3-(trifluoromethyl)cyclobutanol;
1-(2-(1,8-naphthyridin-3-yl)thieno[2,3-d]pyrimidin-6-yl)-3-(trifluoromethyl)cyclobutanol
3-(difluoromethyl)-1-(5-(2-methyl-2H-pyrazolo[3,4-b]pyridin-5-yl)thiazolo[5,4-b]pyridin-2-yl)cyclobutanol; or
1-(2-([1,2,4]triazolo[1,5-a]pyrimidin-6-yl)thieno[2,3-d]pyrimidin-6-yl)-3-(trifluoromethyl)cyclobutan-1-ol.

Provided herein as Embodiment 290 is the compound or salt of Embodiment 1, wherein the compound is:

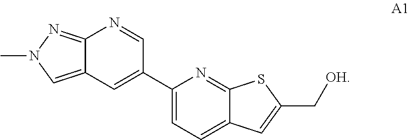 [SMILES: Cn1cc2cc(-c3ccc4cc(CO)sc4n3)cnc2n1]

Provided herein as Embodiment 291 is the compound or salt of Embodiment 1, wherein the compound is:

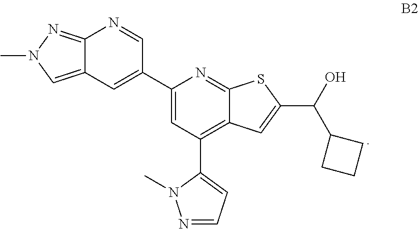 [SMILES: Cn1cc2cc(-c3cc(-c4ccnn4C)c4cc(C(O)C5CCC5)sc4n3)cnc2n1]

Provided herein as Embodiment 292 is the compound or salt of Embodiment 1, wherein the compound is:

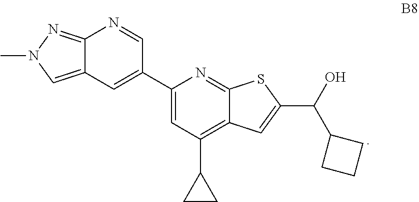 [SMILES: Cn1cc2cc(-c3cc(C4CC4)c4cc(C(O)C5CCC5)sc4n3)cnc2n1]

Provided herein as Embodiment 293 is the compound or salt of Embodiment 1, wherein the compound is:

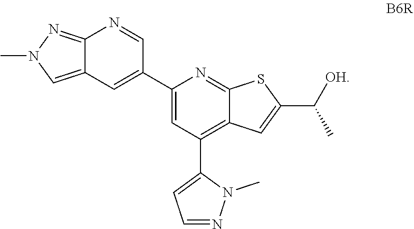 [SMILES: C[C@@H](O)c1cc2c(-c3ccnn3C)cc(-c3cnc4nn(C)cc4c3)nc2s1]

Provided herein as Embodiment 294 is the compound or salt of Embodiment 1, wherein the compound is:

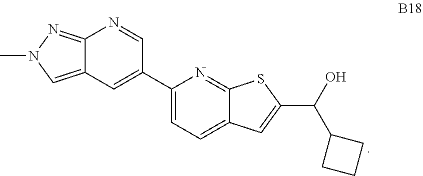 [SMILES: Cn1cc2cc(-c3ccc4cc(C(O)C5CCC5)sc4n3)cnc2n1]

Provided herein as Embodiment 295 is the compound or salt of Embodiment 1, wherein the compound is:

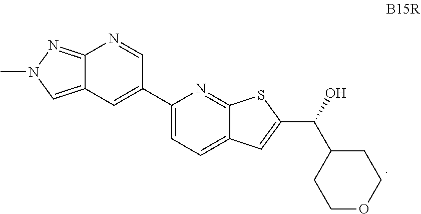 [SMILES: Cn1cc2cc(-c3ccc4cc([C@H](O)C5CCOCC5)sc4n3)cnc2n1]

Provided herein as Embodiment 296 is the compound or salt of Embodiment 1, wherein the compound is:

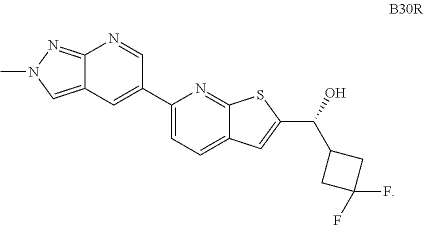 [SMILES: Cn1cc2cc(-c3ccc4cc([C@H](O)C5CC(F)(F)C5)sc4n3)cnc2n1]

Provided herein as Embodiment 297 is the compound or salt of Embodiment 1, wherein the compound is:

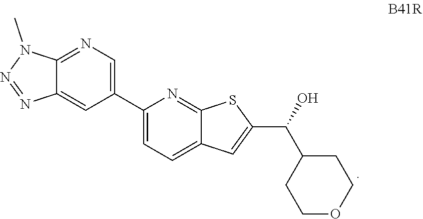 [SMILES: Cn1nnc2cc(-c3ccc4cc([C@H](O)C5CCOCC5)sc4n3)cnc21]

Provided herein as Embodiment 298 is the compound or salt of Embodiment 1, wherein the compound is:

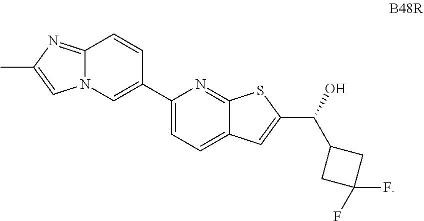 [SMILES: Cc1cn2cc(-c3ccc4cc([C@H](O)C5CC(F)(F)C5)sc4n3)ccc2n1]

Provided herein as Embodiment 299 is the compound or salt of Embodiment 1, wherein the compound is:

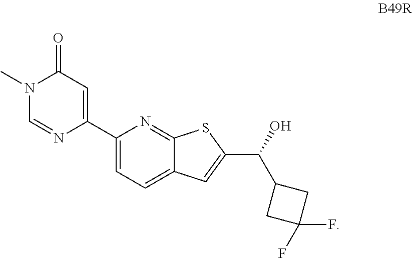 [SMILES: Cn1cnc(-c2ccc3cc([C@H](O)C4CC(F)(F)C4)sc3n2)cc1=O]

Provided herein as Embodiment 300 is the compound or salt of Embodiment 1, wherein the compound is:

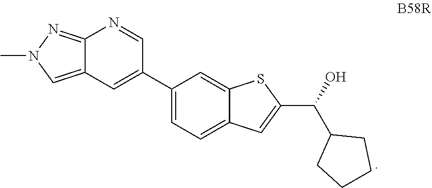 [SMILES: Cn1cc2cc(-c3ccc4cc([C@H](O)C5CCCC5)sc4c3)cnc2n1]

Provided herein as Embodiment 301 is the compound or salt of Embodiment 1, wherein the compound is:

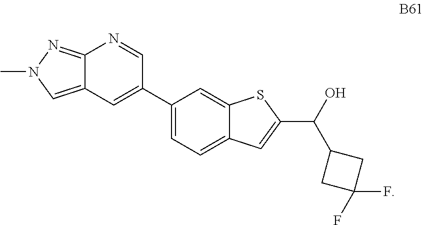 [SMILES: Cn1cc2cc(-c3ccc4cc(C(O)C5CC(F)(F)C5)sc4c3)cnc2n1]

Provided herein as Embodiment 302 is the compound or salt of Embodiment 1, wherein the compound is:

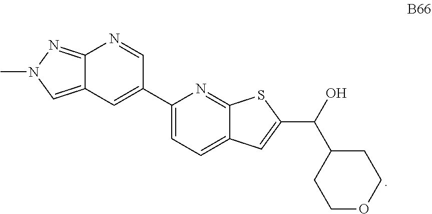 [SMILES: Cn1cc2cc(-c3ccc4cc(C(O)C5CCOCC5)sc4n3)cnc2n1]

Provided herein as Embodiment 303 is the compound or salt of Embodiment 1, wherein the compound is:

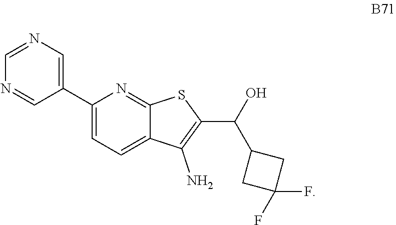 [SMILES: Nc1c(C(O)C2CC(F)(F)C2)sc2nc(-c3cncnc3)ccc12]

Provided herein as Embodiment 304 is the compound or salt of Embodiment 1, wherein the compound is:

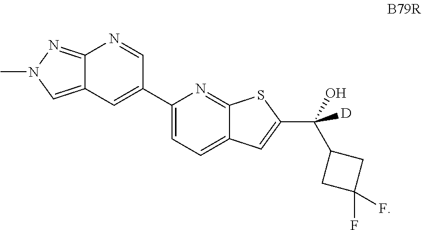 [SMILES: [2H][C@](O)(c1cc2ccc(-c3cnc4nn(C)cc4c3)nc2s1)C1CC(F)(F)C1]

Provided herein as Embodiment 305 is the compound or salt of Embodiment 1, wherein the compound is:

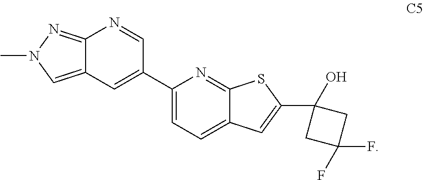 [SMILES: Cn1cc2cc(-c3ccc4cc(C5(O)CC(F)(F)C5)sc4n3)cnc2n1]

Provided herein as Embodiment 306 is the compound or salt of Embodiment 1, wherein the compound is:

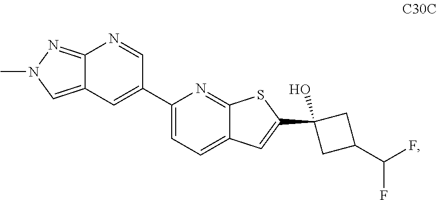

Provided herein as Embodiment 307 is the compound or salt of Embodiment 1, wherein the compound is:

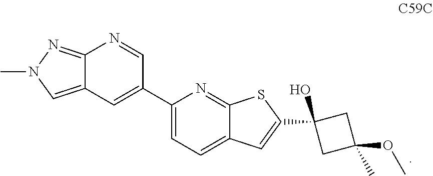

Provided herein as Embodiment 308 is the compound or salt of Embodiment 1, wherein the compound is:

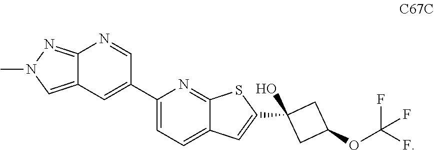 [SMILES: Cn1cc2cc(-c3ccc4cc([C@]5(O)C[C@@H](OC(F)(F)F)C5)sc4n3)cnc2n1]

Provided herein as Embodiment 309 is the compound or salt of Embodiment 1, wherein the compound is:

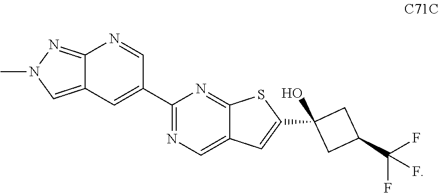 [SMILES: Cn1cc2cc(-c3ncc4cc([C@]5(O)C[C@@H](C(F)(F)F)C5)sc4n3)cnc2n1]

Provided herein as Embodiment 310 is the compound or salt of Embodiment 1, wherein the compound is:

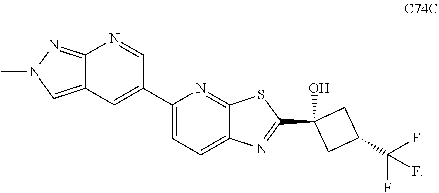 [SMILES: Cn1cc2cc(-c3ccc4nc([C@]5(O)C[C@@H](C(F)(F)F)C5)sc4n3)cnc2n1]

Provided herein as Embodiment 311 is the compound or salt of Embodiment 1, wherein the compound is:

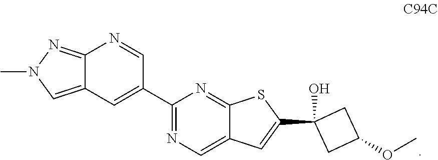

Provided herein as Embodiment 312 is the compound or salt of Embodiment 1, wherein the

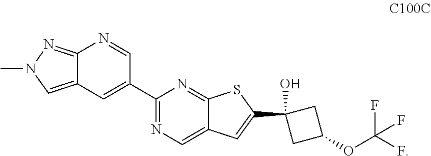 [SMILES: Cn1cc2cc(-c3ncc4cc([C@]5(O)C[C@@H](OC(F)(F)F)C5)sc4n3)cnc2n1]

Provided herein as Embodiment 313 is the compound of any one of Embodiments 1 to 312.

If the stereochemistry of a structure or a portion of a structure disclosed herein is not explicitly shown (e.g., such as with dashed or bold lines), then the structure or portion of structure is either achiral or interpreted as being any of the possible stereoisomers of the structure or portion of the structure. If the stereochemistry of a structure or portion of a structure is explicitly shown, a single stereoisomer of the structure or portion of the structure is represented, with the understanding that the stereochemistry of the structure or portion of the structure may have been arbitrarily assigned.

The term “stereoisomer” or “stereoisomerically pure” compound as used herein refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of other enantiomers or diastereomers of the compound. A compound as disclosed in any one of Embodiments 1 to 289 may be stereoisomerically pure or stereoisomerically enriched.

A typical stereoisomerically enriched compound comprises greater than about 50% by weight of one stereoisomer of the compound and equal or less than about 50% by weight of other stereoisomers of the compound, greater than about 60% by weight of one stereoisomer of the compound and equal or less than about 40% by weight of other stereoisomers of the compound, greater than about 70% by weight of one stereoisomer of the compound and equal or less than about 30% by weight of other stereoisomers of the compound, greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, greater than about 97% by weight of one stereoisomer of the compound and equal or less than about 3% by weight of other stereoisomers of the compound, greater than about 99% by weight of one stereoisomer of the compound and equal or less than about 1% by weight of other stereoisomers of the compound, or greater than about 99.9% by weight of one stereoisomer of the compound and equal or less than about 0.10% by weight of other stereoisomers of the compound.

This disclosure also encompasses the pharmaceutical compositions comprising stereoisomerically pure forms (or enriched forms) and the use of stereoisomerically pure forms (or enriched forms) of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. See, for example, Jacques et al., Enantiomers, Racemates and Resolutions (Wiley-Interscience, New York, 1981); Wilen et al., Tetrahedron 33:2725; Eliel, Stereochemistry of Carbon Compounds (McGraw-Hill, NY, 1962); and Wilen, Tables of Resolving Agents and Optical Resolutions, page 268 (Eliel, Ed., Univ. of Notre Dame Press, Notre Dame, IN, 1972).

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula. Accordingly, the scope of the instant disclosure is to be understood to encompass all tautomeric forms of the compounds disclosed herein. Exemplary tautomers include enol and imine forms, the keto and enamine forms, and geometric isomers and mixtures thereof. A tautomer is one of two or more structural isomers that exist in equilibrium and are readily converted from one isomeric form to another. This reaction results in the formal migration of a hydrogen atom accompanied by a switch of adjacent conjugated double bonds.

As discussed elsewhere herein, the compounds disclosed herein and the stereoisomers, tautomers, and isotopically-labelled forms thereof or a pharmaceutically acceptable salt of any of the foregoing may exist in solvated or unsolvated forms.

In several embodiments, the compound of Formula (A) (or any one of Formulae (I), (II), (III), (IV), (V), (VI), (VII), (VIII), (IX), (X), (XI), (XII), (IAi), (IAii), (IBi), (IBii), (IBiii), (ICi), (ICii), (ICiii), (IDi), (IDii), (I-1), (I-2), (I-3), (III-1), (III-2), (III-3), (III-4), (III-5), (IV-1), (IV-2), (IV-3), (IV-4), (XI-1), (XI-2), (XII-1), or any other compound disclosed herein) can be characterized by its ability to inhibit the enzymatic activity of 15-PGDH (e.g., recombinant 15-PGDH) in an assay. In several embodiments, the assay is a cellular assay or a cell-free binding assay. In several embodiments, the compound of Formula (A) (or any one of Formulae (I), (II), (III), (IV), (V), (VI), (VII), (VIII), (IX), (X), (XI), (XII), (IAi), (IAii), (IBi), (IBii), (IBiii), (ICi), (ICii), (ICiii), (IDi), (IDii), (I-1), (I-2), (I-3), (III-1), (III-2), (III-3), (III-4), (III-5), (IV-1), (IV-2), (IV-3), (IV-4), (XI-1), (XI-2), (XII-1), or any other compound disclosed herein) inhibits the enzymatic activity of 15-PGDH in the assay at an IC₅₀ of less than or equal to about: 1 μM, 250 nM, 50 nM, 20 nM, 10 nM, 5 nM, 2.5 nM 1 nM, 0.1 nM, 0.01 nM, 1 μM, or ranges including and/or spanning the aforementioned values. In several embodiments, the compound of Formula (A) (or any one of Formulae (I), (II), (III), (IV), (V), (VI), (VII), (VIII), (IX), (X), (XI), (XII), (IAi), (IAii), (IBi), (IBii), (IBiii), (ICi), (ICii), (ICiii, (IDi), (IDii), (I-1), (I-2), (I-3), (III-1), (III-2), (III-3), (III-4), (III-5), (IV-1), (IV-2), (IV-3), (IV-4), (XI-1), (XI-2), (XII-1), or any other compound disclosed herein) inhibits the enzymatic activity of recombinant 15-PGDH at an IC₅₀ ranging from about 20 nM to about 0.01 pM, from about 20 nM to about 0.01 nM, from about 20 nM to about 0.1 nM, from about 20 nM to about 1 nM, from about 10 nM to about 0.01 pM, from about 10 nM to about 0.01 nM, from about 10 nM to about 0.1 nM, from about 10 nM to about 1 nM, from about 5 nM to about 0.01 pM, from about 5 nM to about 0.01 nM, from about 5 nM to about 0.1 nM, or from about 5 nM to about 1 nM.

In several embodiments, the IC₅₀ is measured using an assay as disclosed in the Examples. In several embodiments, the IC₅₀ for inhibition of 15-PGDH is measured using a cell-free binding assay. In several embodiments, recombinant 15-PGDH enzymatic assays are performed in a 25 μL volume of reaction buffer containing 50 mM Tris, pH 7.5, 0.01% Tween-20 and 100 μM DTT in a 384-well microtiter plate. In several embodiments, concentration-response experiments with test compounds are performed at 22 concentrations from 2-fold serial dilutions in DMSO. In several embodiments, the test compounds are pre-incubated with 15-PGDH for 15 minutes at room temperature. In several embodiments, thereafter, PGE2 and β-NAD+ are added to initiate the 15-PGDH reaction. After 60 minutes at room temperature, the reaction is quenched and NADH signal was measured using a microtiter plate reader.

In several embodiments, the compound of Formula (A) (or any one of Formulae (I), (II), (III), (IV), (V), (VI), (VII), (VIII), (IX), (X), (XI), (XII), (IAi), (IAii), (IBi), (IBii), (IBiii1), (ICi), (ICIi), (ICiii), (IDi), (IDii), (I-1), (I-2), (I-3), (III-1), (III-2), (III-3), (III-4), (III-5), (IV-1), (IV-2), (IV-3), (IV-4), (XI-1), (XI-2), (XII-1), or any other compound disclosed herein) can be characterized by its low inhibition of acetylcholinesterase (ACHE). In several embodiments, inhibition of ACHE may be measured using the method described in Ellman G. L., et al., A new and rapid colorimetric determination of acetylcholinesterase activity. Biochem Pharmacol. 1961; 7:88-95. In several embodiments, ACHE inhibition is measured by colorimetric detection of conversion of acetylthiocholine to thiocholine using a compound concentration of 10 μM under the Ellman conditions. In several embodiments, the substrate (acetylthiocholine; ATch) is provided at a concentration of 400 pM. In several embodiments, the ACHE enzyme is provided at a concentration of 1 mU. In several embodiments, dithiobisnotrobenzoate (DTNB) is provided at a concentration of 500 μM. In several embodiments, the reaction is carried out in a buffer of 8 mM NaH₂PO₄/Na₂HPO₄, 20 mM NaCl, 0.06% Triton and 0.8 mM EDTA and about 1 mU enzyme (ACHE). In several embodiments, the test compound is added to the buffer comprising enzyme. In several embodiments, the enzymatic reaction is initiated by addition of 400 μM of the substrate ATch and 500 μM DTNB. In this reaction ATch is transformed in thiocholine that will react with DTNB and forms an anion, 5-thio-2-nitrobenzoate that is yellow. In several embodiments, the absorbance is measured immediately (t=0) at λ=405 nm using a microplate reader. In several embodiments, the plate reader is an Envision, Perkin Elmer plate reader. In several embodiments, this measurement allows to verify compound interference with the spectrophotometric detection at this wavelength. After 30 min incubation, a second measurement of the absorbance is made. The enzyme activity is determined by subtracting the signal measured at t=0 from that measured at t=30. In several embodiments, water is used as a control (substituted for the experimental compound). In several embodiments, a compound of Formula (A) (or any one of Formulae (I), (II), (III), (IV), (V), (VI), (VII), (VIII), (IX), (X), (XI), (XII), (IAi), (IAii), (IBi), (IBii), (IBiii), (ICi), (ICii), (ICiii), (IDi), (IDii), (I-1), (I-2), (I-3), (III-1), (III-2), (III-3), (111-4), (111-5), (IV-1), (IV-2), (IV-3), (IV-4), (XI-1), (XI-2), (XII-1), or any other compound disclosed herein) resulted in no detectable inhibition of ACHE at a 10 pM test compound concentration. In several embodiments, a compound of Formula (A) (or any one of Formulae (I), (II), (III), (IV), (V), (VI), (VII), (VIII), (IX), (X), (XI), (XII), (IAi), (IAii), (IBi), (IBii), (IBiii), (ICi), (ICIi), (ICiii), (IDi), (IDii), (I-1), (I-2), (I-3), (III-1), (III-2), (III-3), (III-4), (III-5), (IV-1), (IV-2), (IV-3), (IV-4), (XI-1), (XI-2), (XII-1), or any other compound disclosed herein) resulted in no detectable inhibition of ACHE at a 5 pM test compound concentration. In several embodiments, a compound of Formula (A) (or any one of Formulae (I), (II), (III), (IV), (V), (VI), (VII), (VIII), (IX), (X), (XI), (XII), (IAi), (IAii), (IBi), (IBii), (IBiii), (ICi), (ICii), (ICiii), (IDi), (IDii), (I-1), (I-2), (I-3), (III-1), (III-2), (III-3), (III-4), (III-5), (IV-1), (IV-2), (IV-3), (IV-4), (XI-1), (XI-2), (XII-1), or any other compound disclosed herein) resulted in no detectable inhibition of ACHE at a 2.5 pM test compound concentration. In several embodiments, a compound of Formula (A) (or any one of Formulae (I), (II), (III), (IV), (V), (VI), (VII), (VIII), (IX), (X), (XI), (XII), (IAi), (IAii), (IBi), (IBii), (IBiii), (ICi), (ICii), (ICiii), (IDi), (IDii), (I-1), (I-2), (I-3), (III-1), (III-2), (III-3), (111-4), (111-5), (IV-1), (IV-2), (IV-3), (IV-4), (XI-1), (XI-2), (XII-1), or any other compound disclosed herein) resulted in no detectable inhibition of ACHE at a 1.0 pM test compound concentration. In several embodiments, at a concentration of test compound of 10 pM, the % inhibition of the enzyme is less than or equal to about 0% (undetectable), 0.05%, 0.1%, 0.5%, 0.75%, 1.0%, 2.0%, 5.0%, or ranges including and/or spanning the aforementioned values.

### Isotopically-Labelled Compounds

Further, the scope of the present disclosure includes all pharmaceutically acceptable isotopically-labelled compounds of the compounds disclosed herein, such as the compounds of Formula (A) (or any one of Formulae (I), (II), (III), (IV), (V), (VI), (VII), (VIII), (IX), (X), (XI), (XII), (IAi), (IAii), (IBi), (IBii), (IBiii), (ICi), (ICii), (ICiii, (IDi), (IDii), (I-1), (I-2), (I-3), (III-1), (III-2), (III-3), (III-4), (111-5), (IV-1), (IV-2), (IV-3), (IV-4), (XI-1), (XI-2), (XII-1), benzothiophenyl alcohols, thienopyridinyl alcohols, or any other compound disclosed herein) wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H (deuterium) and ³H (tritium), carbon, such as ¹¹C, ¹³C and ¹⁴C, chlorine, such as ³⁶CI, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of Formula (A), for example, those incorporating a radioactive isotope, are useful in drug and/or substrate tissue distribution studies. The radioactive isotopes tritium (H) and carbon-14 (¹⁴C) are particularly useful for this purpose in view of their ease of incorporation and ready means of detection. Substitution with isotopes such as deuterium (2H or D) may afford certain therapeutic advantages resulting from greater metabolic stability, for example, increased in vivo half-life or reduced dosage requirements, and hence may be advantageous in some circumstances. In several embodiments, the compounds herein are deuterated at metabolic hot spots (e.g., where certain hydrogen atoms of a structure are more metabolically labile than other hydrogens present on the molecule). Substitution with positron emitting isotopes, such as ¹¹C, ¹⁸F, ¹⁵O and ¹³N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying General Synthetic Schemes and Examples using an appropriate isotopically-labelled reagent in place of the non-labelled reagent previously employed.

### Solvates

As discussed above, the compounds disclosed herein and the stereoisomers and isotopically-labelled forms thereof or a pharmaceutically acceptable salt of any of the foregoing may exist in solvated or unsolvated forms. The term “solvate” as used herein refers to a molecular complex comprising a compound or a pharmaceutically acceptable salt thereof as described herein and a stoichiometric or non-stoichiometric amount of one or more pharmaceutically acceptable solvent molecules. If the solvent is water, the solvate is referred to as a “hydrate.” Accordingly, the scope of the instant disclosure is to be understood to encompass all solvents of the compounds disclosed herein and the stereoisomers, tautomers and isotopically-labelled forms thereof or a pharmaceutically acceptable salt of any of the foregoing.

### Formulation and Route of Administration

While it may be possible to administer a compound disclosed herein alone in the uses described, the compound administered normally will be present as an active ingredient in a pharmaceutical composition. Thus, in several embodiments, provided herein is a pharmaceutical composition comprising a compound disclosed herein in combination with one or more pharmaceutically acceptable excipients, such as diluents, carriers, adjuvants and the like, and, if desired, other active ingredients. In several embodiments, a pharmaceutical composition comprises a therapeutically effective amount of a compound disclosed herein.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. The pharmaceutical composition is typically made in the form of a dosage unit containing a particular amount of the active ingredient.

Provided herein as Embodiment 314 is pharmaceutical composition comprising the compound or salt of any one of Embodiments 1 to 312 or a compound of Embodiment 313, and a pharmaceutically acceptable excipient.

Further, this disclosure encompasses pharmaceutical compositions comprising mixtures of any of the compounds disclosed herein and one or more other active agents disclosed herein.

### Use of Compounds and Compositions, Medicaments for Use, and Methods of Treatment

As discussed herein, the compounds described herein are to be understood to include all stereoisomers, tautomers, or pharmaceutically acceptable salts of any of the foregoing or solvates of any of the foregoing. Accordingly, the scope of the methods and uses provided in the instant disclosure is to be understood to encompass also methods and uses employing all such forms. Besides being useful for human treatment, the compounds provided herein may be useful for veterinary treatment of companion animals, exotic animals and farm animals, including mammals, rodents, and the like. For example, animals including horses, dogs, and cats may be treated with compounds provided herein.

In several embodiments, as disclosed elsewhere herein, a method of treating a patient is provided. In several embodiments, the method comprises administering a therapeutic amount of a 15-PGDH inhibitor compound (e.g., of any one of Formulae (A), (I), (II), (III), (IV), (V), (VI), (VII), (VIII), (IX), (X), (XI), (XII), (IAi), (IAii), (IBi), (IBii), (IBiii), (ICi), (ICii), (ICiii), (IDi), (IDii), (I-1), (I-2), (I-3), (III-1), (III-2), (III-3), (III-4), (III-5), (IV-1), (IV-2), (IV-3), (IV-4), (XI-1), (XI-2), (XII-1), a benzothiophenyl alcohol, a thienopyridinyl alcohol, or any other compound disclosed herein) to a patient.

In several embodiments, after administration of a 15-PGDH inhibitor to the patient, the patient's response is measured. In several embodiments, a beneficial effect of the 15-PGDH inhibitors may be assessed by a reduction in one or more inflammatory biomarkers in a relevant sample from the subject. In several embodiments, the inflammatory biomarker may comprise or consist of one or more of cytokines or inflammatory cytokines (e.g., those associated with fibrosis). Such cytokines can include, for example, ′HHb, MIP2 (e.g., CCL3 or CCL4), IFN5, TGFP, TNFa, IL-6, MCP-1, IL2, and IL-10 in BAL fluid. Methods for measuring the amount of such biomarkers, include but are not limited to ELISAs. In several embodiments, the biomarker is an eicosanoid or a PGE2 metabolite, such as, prostaglandin E2 (PGE2), prostaglandin E metabolite (PGE-M; tetranor-PGEM), 15-keto prostaglandin E 2 (15-keto-PGE2), prostaglandin F2α PGF2α, 6-keto prostaglandin F1α (6-keto-PGF1α), prostaglandin D2 (PGD2), prostaglandin J2 (PGJ2), tetranor-PGE1 (TN-E), thromboxane B2 (TXB2), leukotriene B4 (LTB4), 15-hydroxyeicosatetraenoic acid (15-HETE), 12-hydroxyeicosatetraenoic acid (12-HETE), 8-hydroxyeicosatetraenoic acid (8-HETE), 5-hydroxyeicosatetraenoic acid (5-HETE), 17-HDA, 12,13-dihydroxy-9Z-octadecenoic acid (12, 13-DiHOME), 9,10-dihydroxy-9Z-octadecenoic acid 9,10-DiHOME, 14,15-dihydroxyeicosatrienoic acid (14,15-DHET), 11,12-dihydroxyeicosatrienoic acid (11,12-DHET), or combinations of the foregoing. In several embodiments, the methods disclosed herein may comprise reducing an amount of one or more biomarkers in a sample from the subject compared to control. In several embodiments, the methods disclosed herein may comprise reducing an amount of one or more biomarkers in a sample from the subject compared to the patient prior to treatment. In several embodiments, the methods disclosed herein may comprise increasing an amount of one or more biomarkers in a sample from the subject compared to control. In several embodiments, the methods disclosed herein may comprise increasing an amount of one or more biomarkers in a sample from the subject compared to the patient prior to treatment. Thus, the levels of these biomarkers may be measured before or after treatment with a 15-PDGH inhibitor as disclosed herein.

In several embodiments, as disclosed elsewhere herein, the patient is one suffering from a 15-PGDH mediated disease or disorder. In several embodiments, the 15-PGDH mediated disease or disorder is inflammatory bowel disease, ulcerative colitis, Crohn's disease, fibrotic disease disorder or condition. In several embodiments, the 15-PGDH mediated disease or disorder is atherosclerosis. In several embodiments, the 15-PGDH mediated disease or disorder is autoimmune disease. In several embodiments, the 15-PGDH mediated disease is systemic sclerosis, multifocal fibrosclerosis, nephrogenic systemic fibrosis, kidney fibrosis, glomerular sclerosis, renal tubulointerstitial fibrosis, progressive renal disease or diabetic nephropathy, cardiac fibrosis, pulmonary fibrosis, glomerulosclerosis pulmonary fibrosis, idiopathic pulmonary fibrosis, silicosis, asbestosis, interstitial lung disease, interstitial fibrotic lung disease, chemotherapy/radiation induced pulmonary fibrosis, endomyocardial fibrosis, deltoid fibrosis, pancreatitis, general fibrosis syndrome characterized by replacement of normal muscle tissue by fibrous tissue in varying degrees, retroperitoneal fibrosis, liver fibrosis, liver cirrhosis, chronic renal failure, myelofibrosis, bone marrow fibrosis, acute fibrosis, or organ specific fibrosis. In several embodiments, the 15-PGDH mediated disease is intestinal ischemia, ischemia, ischemic brain disease, ischemic heart disease, ischemic peripheral vascular disease, ischemic placenta, ischemic renal disease, ischemic vascular disease, ischemic-reperfusion injury, limb ischemia, lower extremity ischemia, myocardial ischemia, organ ischemia, peripheral ischemia, tissue ischemia, transient ischemic attack (TIA), and wounds to tissues or organs.

Provided herein as Embodiment 315 is a compound or salt according to any one of Embodiments 1 to 312, the compound of Embodiment 313, or the pharmaceutical composition of Embodiment 314, for use as a medicament.

Provided herein as Embodiment 316 is a compound or salt according to any one of Embodiments 1 to 312, the compound of Embodiment 313, or the pharmaceutical composition of Embodiment 314 for use in treating a 15-PGDH mediated disease or disorder.

Provided herein as Embodiment 317 is a compound or salt according to any one of Embodiments 1 to 312, the compound of Embodiment 313, or the pharmaceutical composition of Embodiment 314 for use in treating inflammatory bowel disease.

Provided herein as Embodiment 318 is a compound or salt according to any one of Embodiments 1 to 312, the compound of Embodiment 313, or the pharmaceutical composition of Embodiment 314 for use in treating ulcerative colitis.

Provided herein as Embodiment 319 is a compound or salt according to any one of Embodiments 1 to 312, the compound of Embodiment 313, or the pharmaceutical composition of Embodiment 314 for use in treating Crohn's disease.

Provided herein as Embodiment 320 is a compound or salt according to any one of Embodiments 1 to 312, the compound of Embodiment 313, or the pharmaceutical composition of Embodiment 314 for use in treating a fibrotic disease, disorder or condition.

Provided herein as Embodiment 321 is a compound, salt, or composition according to Embodiment 320, wherein the fibrotic disease is characterized, in whole or in part, by the excess production of fibrous material, including excess production of fibrotic material within the extracellular matrix, or the replacement of normal tissue elements by abnormal, non-functional, and/or excessive accumulation of matrix-associated components.

Provided herein as Embodiment 322 is a compound, salt, or composition according to Embodiment 320 or 321, wherein the fibrotic disease is systemic sclerosis, multifocal fibrosclerosis, nephrogenic systemic fibrosis, kidney fibrosis, glomerular sclerosis, renal tubulointerstitial fibrosis, progressive renal disease or diabetic nephropathy, cardiac fibrosis, pulmonary fibrosis, glomerulosclerosis pulmonary fibrosis, idiopathic pulmonary fibrosis, silicosis, asbestosis, interstitial lung disease, interstitial fibrotic lung disease, chemotherapy/radiation induced pulmonary fibrosis, endomyocardial fibrosis, deltoid fibrosis, pancreatitis, general fibrosis syndrome characterized by replacement of normal muscle tissue by fibrous tissue in varying degrees, retroperitoneal fibrosis, liver fibrosis, liver cirrhosis, chronic renal failure; myelofibrosis, bone marrow fibrosis, acute fibrosis, or organ specific fibrosis.

Provided herein as Embodiment 323 is a compound, salt, or composition according to Embodiment 320 or 321, wherein the fibrotic disease is idiopathic pulmonary fibrosis.

Provided herein as Embodiment 324 is a compound, salt, or composition according to Embodiment 320 or 321, wherein the fibrotic disease is kidney fibrosis.

Provided herein as Embodiment 325 is a compound, salt, or composition according to Embodiment 320 or 321, wherein the fibrotic disease is liver fibrosis.

Provided herein as Embodiment 326 is a compound or salt according to any one of Embodiments 1 to 312, the compound of Embodiment 313, or the pharmaceutical composition of Embodiment 314 for use in treating one or more of atherosclerotic cardiovascular disease, autoimmune disease, intestinal ischemia, ischemia, ischemic brain disease, ischemic heart disease, ischemic peripheral vascular disease, ischemic placenta, ischemic renal disease, ischemic vascular disease, ischemic-reperfusion injury, limb ischemia, lower extremity ischemia, myocardial ischemia, organ ischemia, peripheral ischemia, tissue ischemia, transient ischemic attack (TIA), and wounds to tissues or organs.

Provided herein as Embodiment 327 is a compound or salt according to any one of Embodiments 1 to 312 or the compound of Embodiment 313, for the manufacture of a medicament for the treatment of a 15-PGDH mediated disease or disorder.

Provided herein as Embodiment 328 is a compound or salt according to any one of Embodiments 1 to 312 or the compound of Embodiment 313, for the manufacture of a medicament for the treatment of inflammatory bowel disease.

Provided herein as Embodiment 329 is a compound or salt according to any one of Embodiments 1 to 312 or the compound of Embodiment 313, for the manufacture of a medicament for the treatment of ulcerative colitis.

Provided herein as Embodiment 330 is a compound or salt according to any one of Embodiments 1 to 312 or the compound of Embodiment 313, for the manufacture of a medicament for the treatment of Crohn's disease.

Provided herein as Embodiment 331 is a compound or salt according to any one of Embodiments 1 to 312 or the compound of Embodiment 313, for the manufacture of a medicament for the treatment of a fibrotic disease.

Provided herein as Embodiment 332 is a compound or salt according to any one of Embodiments 1 to 312 or the compound of Embodiment 313, for the manufacture of a medicament for the treatment of one or more of atherosclerotic cardiovascular disease, autoimmune disease, intestinal ischemia, ischemia, ischemic brain disease, ischemic heart disease, ischemic peripheral vascular disease, ischemic placenta, ischemic renal disease, ischemic vascular disease, ischemic-reperfusion injury, limb ischemia, lower extremity ischemia, myocardial ischemia, organ ischemia, peripheral ischemia, tissue ischemia, transient ischemic attack (TIA), and wounds to tissues or organs.

Provided herein as Embodiment 333 is a method of treating a 15-PGDH mediated disease in a subject in need thereof, the method comprising:
administering to the subject a therapeutically acceptable amount of a compound or salt according to any one of Embodiments 1 to 312, the compound of Embodiment 313, or the pharmaceutical composition of Embodiment 314.

Provided herein as Embodiment 334 is a method of treating intestinal, gastrointestinal, or bowel disorders in a subject in need thereof, the method comprising:
administering to the subject a therapeutically acceptable amount of a compound or salt according to any one of Embodiments 1 to 312, the compound of Embodiment 313, or the pharmaceutical composition of Embodiment 314.

Provided herein as Embodiment 335 is the method of Embodiment 333 or 334, wherein the disease or disorder comprises at least one of ulcerative colitis, inflammatory bowel disease, and Crohn's disease.

### Combinations

In several embodiments, the 15-PGDH inhibitor compounds disclosed herein (including those of Formula (A), Formula (I), other formulae disclosed herein, benzothiophenyl alcohols, 5,6-fused thieno-heterocyclyl alcohols, and/or thienopyridinyl alcohols) can be provided in combination with other therapeutic agents.

In several embodiments, a 15-PGDH inhibitor as disclosed herein is used in combination with a tumor necrosis factor (TNF) inhibitor (e.g., TNF-α inhibitor). In several embodiments, the TNF inhibitors can include, but are not limited to, anti-TNF alpha antibodies (e.g., infliximab, adalimumab, certolizumab pegol, and golimumab), receptor-construct fusion proteins (such as etanercept), or small molecules, such as, but not limited to, pomalidomide, thalidomide, lenalidomide and bupropion.

In several embodiments, a 15-PGDH inhibitor as disclosed herein is used in combination with a corticosteroid. In several embodiments, the corticosteroid is aclovate, alclometasone dipropionate, amcinafel, amcinafide, amcinonide, aristocort A, augmented betamethasone dipropionate, beclamethasone, beclopmethasone dipropionate, betamethasone, betamethasone benzoate, betamethasone-17-benzoate, betamethasone dipropionate, betamethasone sodium phosphate and acetate, betamethasone valerate, betamethasone-17-valerate, chloroprednisone, clobetasol propionate, clobetasone propionate, clocortelone, cordran, corticosterone, cortisol, cortisol acetate, cortisol cypionate, cortisol sodium phosphate, cortisol sodium succinate, cortisone, cortisone acetate, cortodoxone, cyclocort, deflazacort, defluprednate, descinolone, desonide, desowen, desoximetasone, desoxycorticosterone acetate, desoxycorticosterone pivalate, 11-desoxycortisol, dexamethasone, dexamethasone acetate, dexamethasone sodium phosphate, dichlorisone, diflorasone diacetate, dihydroxycortisone, diprolen, diprolene, diprosone, esters of betamethasone, florone, flucetonide, flucloronide, flucortolone, fludrocortisone, fludrocortisone acetate, flumethalone, flumethasone, flumethasone pivalate, flunisolide, fluocinolone acetonide, fluocinolone acetonide acetate, fluocinonide, fluorametholone, fluorocortisone, fluperolone, fluprednisolone, flurandrenolide, fluroandrenolone acetonide, fluticasone propionate, fuprednisolone, halcinonide, halobetasol propionate, halog, hydrocortamate, hydrocortisone, hydrocortisone acetate, hydrocortisone butyrate, hydrocortisone valerate, hydrocortisone-17-valerate, kenalog, lidex, locold, locorten, maxiflor, medrysone, meprednisone, methylprednisolone, 6α-methylprednisolone, methylprednisolone acetate, methylprednisolone sodium succinate, methylprednisone, mometasone furoate, paramethasone, paramethasone acetate, prednidone, prednisone, prednisolone, prednisolone acetate, prednisolone sodium phosphate, prednisolone sodium succinate, prednisolone tebutate, prednisone, psorcon, synalar, temovate, tetrahydrocortisol, topicort, topicort LP, triamcinolone, triamcinolone acetonide, triamcinolone diacetate, triamcinolone hexacotonide, tridesilone, valisone, westcort, or a combination of the foregoing.

In several embodiments, each constituent of the therapeutic combination can be administered in using the same route or a different route. For example, as disclosed elsewhere herein, the 15-PGDH inhibitors compounds disclosed herein may be administered by any suitable route in the form of a pharmaceutical composition adapted to such a route and in a dose effective for the treatment intended. Each constituent of the therapeutic combination can, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The individual therapeutics comprising the combination can be administered substantially simultaneously using any of the disclosed routes for any of the disclosed methods of treatment (e.g., methods of treating IBD, etc.). The individual therapeutics comprising the combination can be administered in a sequential manner using any of the disclosed routes for any of the disclosed methods of treatment (e.g., methods of treating IBD, etc.). The individual therapeutics comprising the combination may be administered together as part of the same composition or as different compositions.

It will be appreciated that, for a particular biologic (e.g., antibody or protein-based therapeutic), those therapeutic agents may be more suitably for administered by injection. Alternatively, small molecules (such as the 15-PGDH inhibitors, benzothiophenyl alcohols, 5,6-fused thieno-heterocyclyl alcohols, and/or thienopyridinyl alcohols disclosed herein) may be suitable for injection or other administration methods (e.g., through other routes, including orally).

### Methods of Manufacture

The compounds provided herein can be synthesized according to the procedures described in this and the following sections. The synthetic methods described herein are merely exemplary, and the compounds disclosed herein may also be synthesized by alternate routes utilizing alternative synthetic strategies, as appreciated by persons of ordinary skill in the art, in view of the teachings of the present disclosure. It should be appreciated that the general synthetic procedures and specific examples provided herein are illustrative only and should not be construed as limiting the scope of the present disclosure in any manner.

Generally, the compounds of Formula (A) and Formula (I) (or any other formula provided herein) can be synthesized according to the following schemes. Suitable reaction conditions, such as, solvent, reaction temperature, and reagents, for the Schemes discussed in this section, may be found in the examples provided herein. As illustrated in schemes that follow, compounds of Formula (A) or Formula (I) can be prepared using intermediates as disclosed herein. As will be understood in the context of the disclosure provided herein, any variables for the intermediates or final compounds provided in this Methods of Manufacture section that are not defined directly in this section may be as defined elsewhere herein. For example, an intermediate of Formulae (A-Int-a), (A-Int-b.1), (A-Int-b.2), (A-Int-b.3), (A-Int-c.3), (A-Int-d.3), (A-Int-e.3), (A-Int-f.3), (A-Int-c.1), etc., or a compound of Formulae (A.1), (A.2), (A.3), (A.4), etc. may have variables as defined in Formula (A) (e.g., as provided in any one of Embodiments 1, 2, etc.). To illustrate, where a boronic ester is provided as B-R², R²may be as defined in Formula (A) or as provided elsewhere herein.

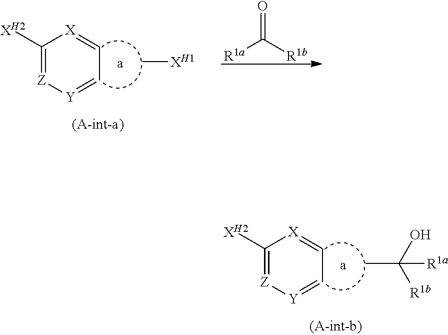

As provided in Scheme Ala, the synthesis of a compound of Formula (A) or Formula (I) may employ an intermediate of Formula (A-int-a) and an intermediate of Formula (A-int-b), in which each of X^{H1} and X^{H2} independently is a suitable halogen atom (e.g., —F, —Cl, —Br, —I, etc.) or a similar reactive group. In several embodiments, each of X^{H1} and X^{H2} is a different halogen atom or another appropriate reactive group. In several embodiments, X^{H}i is —Br, —I, or —H. In several embodiments, X^{H2} is —Br, —Cl, —OTf, -OTs, or -OMs, or another appropriate moiety for Suzuki or Stille coupling.

In several embodiments, the compound of Formula (A-int-a) is reacted with compound comprising R^{1a} and R^{1b}. In several embodiments, the compound comprising R^{1a} and R^{1b} is a ketone or an aldehyde (e.g., where R^{1a} is —H). In several embodiments, the reaction is performed in the presence of metalating agent (e.g., n-butyllithium) and solvent. In several embodiments, the metalating agent exchanges X^{H1} with a metal (e.g., Li). In several embodiments, a compound of Formula (A-int-b) is provided upon nucleophilic addition (e.g., 1,2-addition) of the compound of Formula (A-int-a) to the carbonyl of the compound comprising R^{1a} and R^{1b}. In several embodiments, the compound of Formula (A-int-b.1) may then be subjected to coupling conditions to provide a compound of Formula (A) or Formula (I), as shown in Scheme Alb.

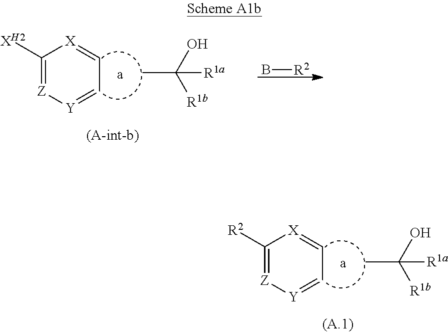

As illustrated in Scheme Alb, compounds of Formula (A.1) (e.g., of Formula (A) where R^{1c} is —H and m is 0) can be prepared by the reaction of an intermediate of Formula (A-int-b), in which X^{H2} represents a suitable halogen atom (e.g., —Cl) or a similar reactive group with an intermediate of B-R², where B represents a suitable reactive moiety, such as a boronic acid, a boronate (or boronic) ester (e.g., a pinacol ester), or a trialkyl stannyl (e.g., tributyl stannyl) group in a cross-coupling reaction (e.g., Suzuki or Stille). In several embodiments, a Suzuki coupling is performed using a palladium or similar transition metal catalyst together with appropriate ligands according to methods described in the literature, and as provided in the Examples below. In several embodiments, the boronic acid group and a boronic ester group (e.g., a pinacol boronate ester) may be represented by the following structures, respectively:

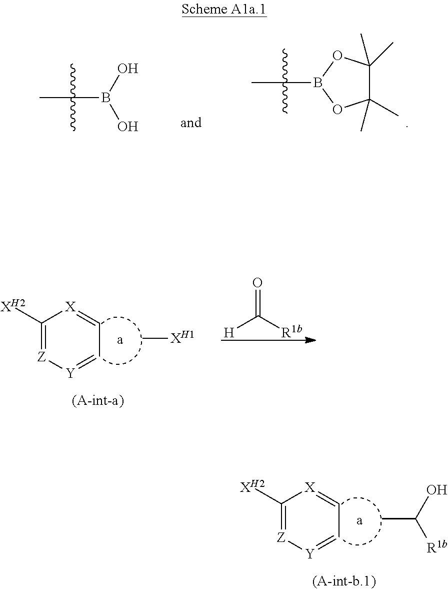

As provided in Scheme Ala.1 and Scheme Alb.1, the synthesis of a compound of Formula (A) or Formula (I) may employ an intermediate of Formula (A-int-a), in which each of X^{H1} and X^{H2} independently is a suitable halogen atom (e.g., —F, —Cl, —Br, —I, etc.) or a similar reactive group. In several embodiments, each of X^{H1} and X^{H2} is a different halogen atom or another appropriate reactive group. In several embodiments, X^{H1} is —Br, —I, or —H. In several embodiments, X^{H2} is —Br, —Cl, -OTf, -OTs, or -OMs, or another appropriate moiety for Suzuki or Stille coupling. In several embodiments, the compound of Formula (A-int-a) is reacted with an aldehyde comprising R^{1b}. In several embodiments, the reaction is performed in the presence of metalating agent (e.g., n-butyllithium) and solvent. In several embodiments, the metalating agent exchanges X^{H1} with a metal (e.g., Li). In several embodiments, a compound of Formula (A-int-b.1) is provided upon nucleophilic addition (e.g., 1,2-addition) to the aldehyde carbonyl. In several embodiments, the compound of Formula (A-int-b.1) may then be subjected to coupling conditions to provide a compound of Formula (A) or Formula (I), as shown in Scheme Alb.

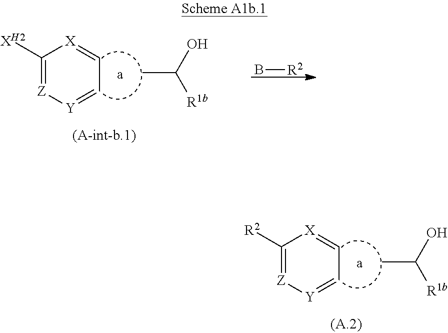

As illustrated in Scheme Alb.1, compounds of Formula (A.2) (e.g., of Formula (A) where R^{1c} and R^{1b} are each —H and m is 0) can be prepared by the reaction of an intermediate of Formula (A-int-b.1), in which X^{H2} represents a suitable halogen atom (e.g., —Cl) or a similar reactive group with an intermediate of B-R², where B represents a suitable reactive moiety, such as a boronic acid, a boronate (or boronic) ester (e.g., a pinacol ester), or a trialkyl stannyl (e.g., tributyl stannyl) group in a cross-coupling reaction (e.g., Suzuki or Stille). In several embodiments, a Suzuki coupling is performed using a palladium or similar transition metal catalyst together with appropriate ligands according to methods described in the literature, and as provided in the Examples below.

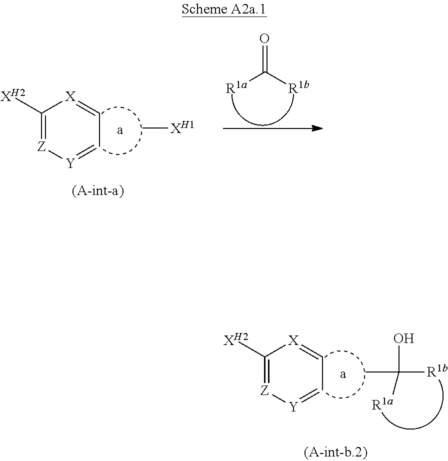

In several embodiments, the compound of Formula (A-int-a) is reacted with a cyclic ketone comprising R^{1a} and R^{1b} (e.g., where the cycle formed by R^{1a} and R^{1b} is as defined in Formula (A), etc.). In several embodiments, the reaction is performed in the presence of metalating agent (e.g., n-butyllithium) and solvent. In several embodiments, the metalating agent exchanges X^{H}i with a metal (e.g., Li). In several embodiments, a compound of Formula (A-int-b.2) is provided upon nucleophilic addition (e.g., 1,2-addition) to the ketone carbonyl. In several embodiments, a compound of Formula (A-int-b.2) is provided.

In several embodiments, as shown in Scheme A2b.1, the compound of Formula (A-int-b.2) may then be subject to coupling conditions to provide a compound of Formula (A.2).

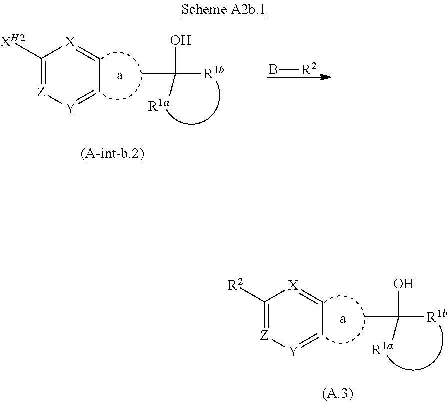

As illustrated in Scheme A2b.1, compounds of Formula (A.3) (e.g., of Formula (A) or Formula (I)) can be prepared by the reaction of an intermediate of Formula (A-int-b.2), in which X^{H2} represents a suitable halogen atom (e.g., —Cl) or a similar reactive group with an intermediate of B-R², where B represents a suitable reactive moiety, such as a boronic acid, boronate (or boronic) ester (e.g., a pinacol ester), or a trialkyl stannyl (e.g., tributyl stannyl) group in a cross-coupling reaction (e.g., Suzuki or Stille). In several embodiments, a Suzuki coupling is performed using a palladium or similar transition metal catalyst together with appropriate ligands according to methods described in the literature, and as provided in the Examples below. In several embodiments, the other variables of the intermediate of Formula (A-Int-b.2) or the compound of Formula (A.3) are as defined anywhere elsewhere herein, including as defined in Formula (A).

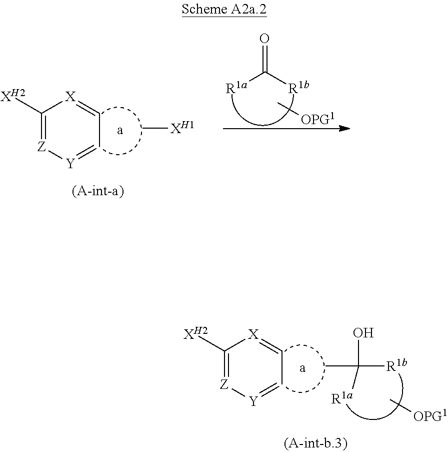

In several embodiments, the compound of Formula (A-int-a) is reacted with a cyclic ketone comprising R^{1a} and R^{1b} where the cycle formed by R^{1a} and R^{1b} includes a protected hydroxyl substituent. In several embodiments, the hydroxyl substituent of the R^{1a} and R^{1b} cycle may later be deprotected to reveal the hydroxyl group. In several embodiments, the hydroxyl group may then be further functionalized to provide a substitution of the R^{1a} and R^{1b} cycle. In several embodiments, the reaction is performed in the presence of metalating agent (e.g., n-butyllithium) and solvent. In several embodiments, the metalating agent exchanges X^{H1} with a metal (e.g., Li). In several embodiments, a compound of Formula (A-int-b.3) is provided upon nucleophilic addition (e.g., 1,2-addition) to the ketone carbonyl. In several embodiments, the protecting group of the hydroxyl (e.g., PG¹), is a suitable hydroxyl protecting group (e.g., benzyl, tert-butyldimethylsilyl (TBS), tert-butyldiphenylsilyl (TBDPS), another silyl protecting group, etc.). In several embodiments, a compound of Formula (A-int-b.3) is provided.

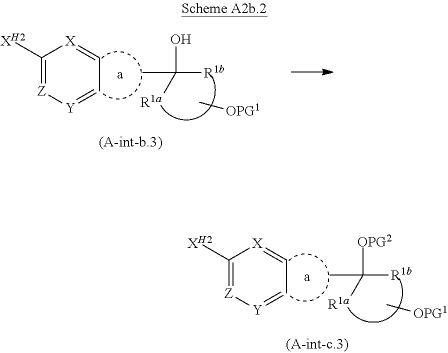

In several embodiments, as shown in Scheme A2b.2, the tertiary alcohol of the compound of Formula (A-int-b.3) may be protected using a second protecting group (PG²). In several embodiments, the protecting group of the tertiary alcohol is an orthogonal protecting group to PG¹. In several embodiments, PG² is an acyl group. In several embodiments, the compound of Formula (A-int-b.3) is reacted with an acylating agent (e.g., acetic anhydride) to afford the compound of Formula (A-int-c.3).

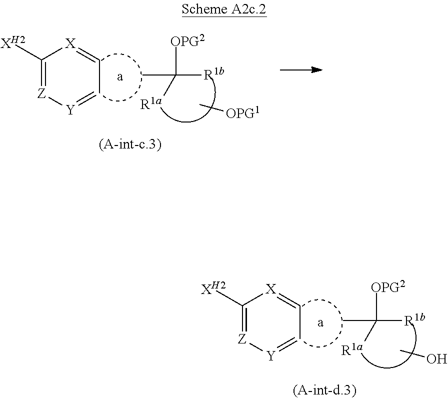

In several embodiments, as shown in Scheme A2c.2, the PG¹ may be removed using a deprotecting step. In several embodiments, where PG¹ is a silyl protecting group, a silyl deprotecting agent is used. In several embodiments, the silyl deprotecting group is tetra-n-butylammonium fluoride (TBAF).

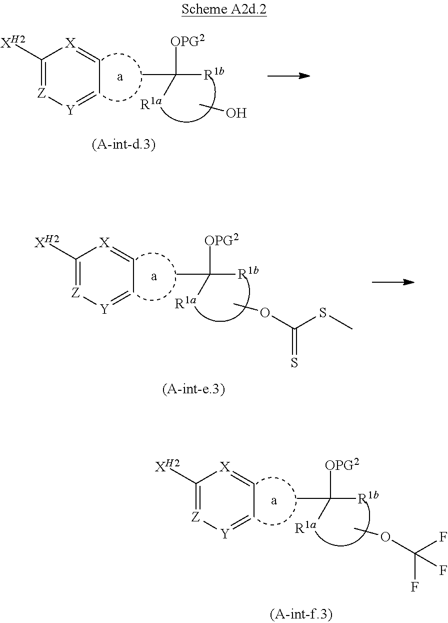

In several embodiments, as shown in Scheme A2d.2, the hydroxyl substituent of the R^{1a} and R^{1b}cycle may be converted to a ((methylthio)carbonothioyl)oxy group through reaction with sodium hydride (NaH) and CS₂ followed by methyl iodide (Mel), providing Intermediate (A-int-e.3). Thereafter, Intermediate (A-int-f.3) is formed by reaction of Intermediate (A-int-e.3) with 1,3-dibromo-5,5-dimethylhydantoin in the presence of HF-pyridine.

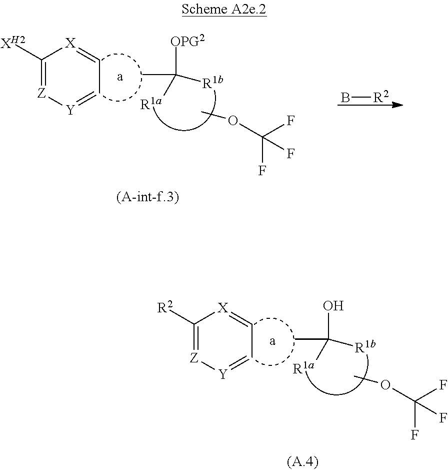

As illustrated in Scheme A2e.2, compounds of Formula (A) or Formula (I) can be prepared by the reaction of an intermediate of Formula (A-int-f.3), in which X^{H2} represents a suitable halogen atom (e.g., —Cl) or a similar reactive group with an intermediate of B-R², where B represents a suitable reactive moiety, such as a boronic acid, boronate (or boronic) ester (e.g., a pinacol ester), or a trialkyl stannyl (e.g., tributyl stannyl) group in a cross-coupling reaction (e.g., Suzuki or Stille). In several embodiments, a Suzuki coupling is performed using a palladium or similar transition metal catalyst together with appropriate ligands according to methods described in the literature, and as provided in the Examples below. In several embodiments, PG² is deprotected using a base (e.g., potassium carbonate) in a one-pot deprotection and coupling reaction to afford the compound of Formula (A.4). Alternatively, the deprotection step may be performed as a separated step after the coupling is performed, as shown in Alt-1-A2e.2.

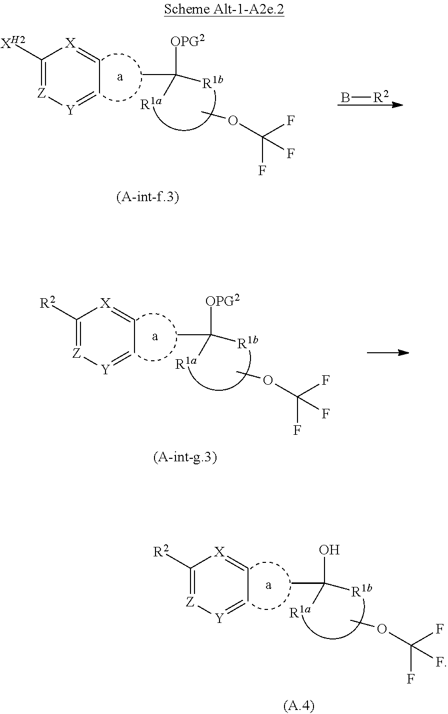

As another alternative, as shown in the Scheme Alt-2-A2e.2, PG² may be deprotected prior to the coupling reaction:

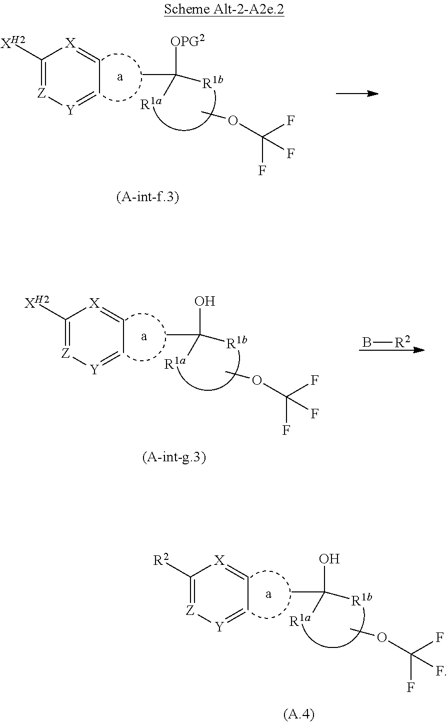

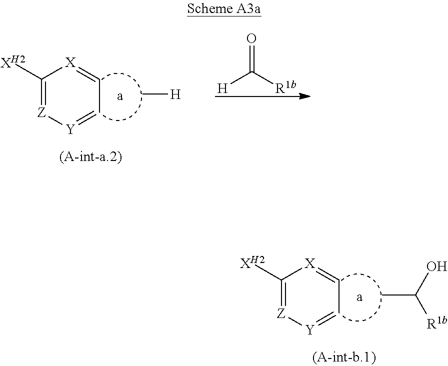

In several embodiments, the compound of Formula (A-int-a.2) is reacted with an aldehyde comprising R^{1b}. In several embodiments, the reaction is performed in the presence of strong base (e.g., lithium diisopropylamide (LDA)) and solvent. In several embodiments, a compound of Formula (A-int-b.1) is provided. As in the compound of Formula (A-int-a.2), the other variables of the compound of Formula (A-int-b.1) may be as defined elsewhere herein, including as defined in Formula (A). In several embodiments, the compound of Formula (A-int-b.1) is then subject to coupling conditions to provide a compound of Formula (A) or Formula (I), as shown in Scheme Alb.

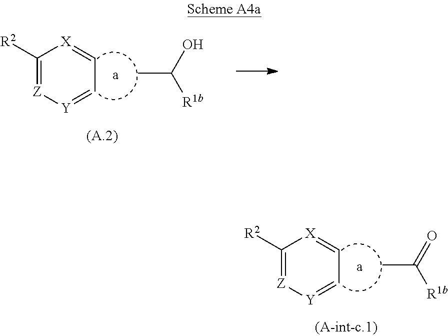

In several embodiments, a secondary alcohol having a structure of Formula (A.2), as shown in Scheme A4a, may be oxidized using an oxidizing agent to provide a ketone. In several embodiments, the oxidizing agent is Dess-Martin periodinane. As in the compound of Formula (A-int-a.2), etc., the other variables of the compound of Formula (A-int-c.1) may be as defined elsewhere herein, including as defined in Formula (A).

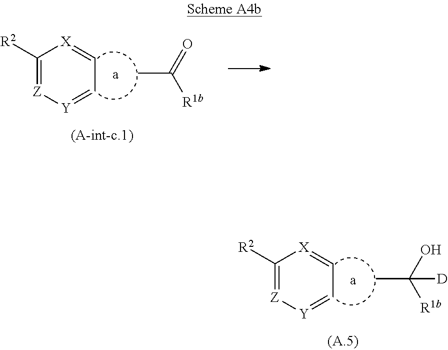

In several embodiments, a secondary alcohol having Formula (A-int-c.1), as shown in Scheme A4b, may be reacted with a deuterating agent to provide a deuterated compound of Formula (A.5). In several embodiments, the deuterating agent is NaBD₄. The other variables of the Formula (A.4) may be as defined elsewhere herein, including as defined in Formula (A).

As illustrated in Schemes 1a-1c, compounds of Formula (A) or Formula (I) can be prepared using intermediate of Formula (I-int-a) and an intermediate of Formula (I-int-b), in which each of X^{H1} and X^{H2} independently is a suitable halogen atom (e.g., —F, —Cl, —Br, —I, etc.) or a similar reactive group. In several embodiments, each of X^{H1} and X^{H2} is a different halogen atom or another appropriate reactive group. In several embodiments, X^{H1} is —Br, —I, or —H. In several embodiments, X^{H2} is —Br, —Cl, -OTf, -OTs, or -OMs, or another appropriate moiety for Suzuki or Stille coupling.

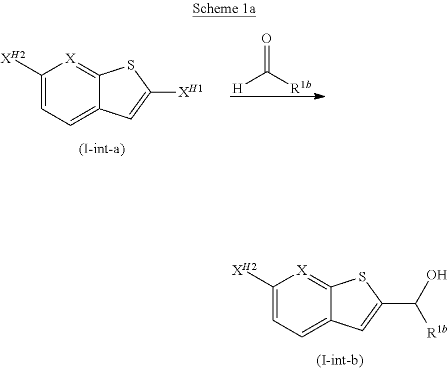

In several embodiments, the compound of Formula (I-int-a) is reacted with an aldehyde comprising R^{1b}. In several embodiments, the reaction is performed in the presence of metalating agent (e.g., n-butyllithium) and solvent. In several embodiments, a compound of Formula (I-int-b) is provided. In several embodiments, the compound of Formula (I-int-b) is then subject to coupling conditions to provide a compound of Formula (A) or Formula (I), as shown in Scheme 1b.

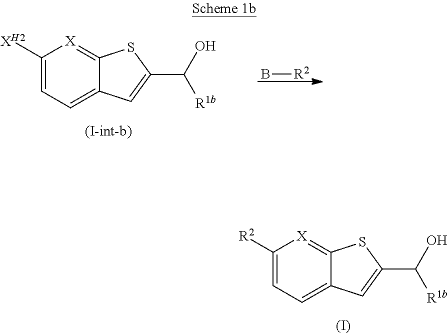

As illustrated in Scheme 1b, compounds of Formula (A) or Formula (I) can be prepared by the reaction of an intermediate of Formula (I-int-b), in which X^{H2} represents a suitable halogen atom (e.g., —Cl) or a similar reactive group with an intermediate of B-R², where B represents a suitable reactive moiety, such as a boronic acid ester (e.g., a pinacol ester) or a trialkyl stannyl (e.g., tributyl stannyl) group in a cross-coupling reaction (e.g., Suzuki or Stille). In several embodiments, a Suzuki coupling is performed using a palladium or similar transition metal catalyst together with appropriate ligands according to methods described in the literature, and as provided in the Examples below.

Scheme 1c provides a representative and nonlimiting illustration of the synthesis performed in Schemes la and 1b:

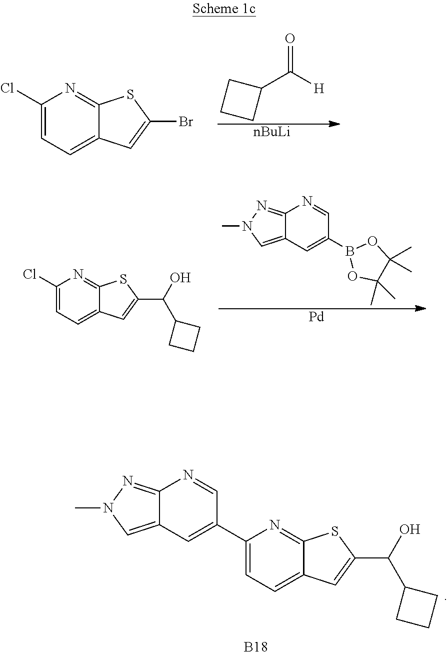

As illustrated in Schemes 2a-d, compounds of Formula (A) or Formula (I) can be prepared using an intermediate of Formula (I-int-c), an intermediate of Formula (I-int-d), an intermediate of Formula (I-int-e), an intermediate of Formula (I-int-f), and an intermediate of Formula (I-int-g). In several embodiments, each X^{H} independently represents a suitable halogen atom (e.g., —Cl, —Br, etc.) or a similar reactive group.

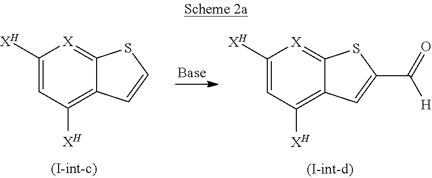

As illustrated in Scheme 2a, an intermediate of Formula (I-int-d) may be prepared from an intermediate of Formula (I-int-e), in which each X^{H} independently represents a suitable halogen atom (e.g., —Cl, —Br, etc.) or a similar reactive group. In several embodiments, the compound of Formula (1-int-c) is reacted with base (e.g., LDA). In several embodiments, the base is added to the compound of Formula (I-int-c) at reduced temperature (e.g., −78° C.). In several embodiments, dimethylformamide (DMF) is added. The reaction is quenched to provide a compound of Formula (I-int-d). In several embodiments, the compound of Formula (I-int-d) is then reacted with a Grignard reagent to provide a compound of Formula (I-int-e), as shown in Scheme 2b.

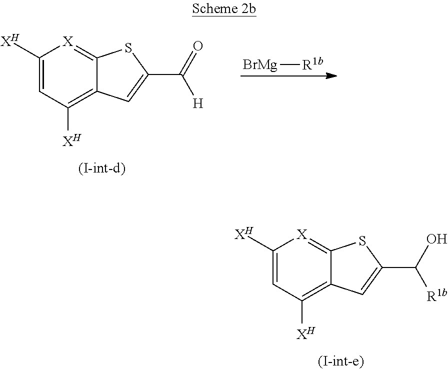

As illustrated in Scheme 2b, an intermediate of Formula (I-int-d) may be reacted with an R^{1b}-magnesium halide (e.g., MgBr) compound in solvent. In several embodiments, the reaction is performed at reduced temperature (e.g., 0° C.). In several embodiments, the compound of Formula (I-int-e) is then subject to cross-coupling conditions to provide a compound of Formula (I-int-f), as shown in Scheme 2c.

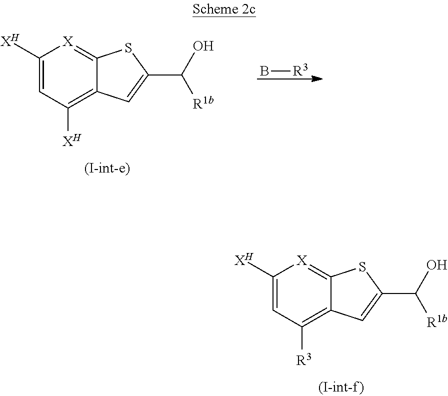

As illustrated in Scheme 2c, under coupling conditions, the intermediate of Formula (I-int-b) provides the intermediate of Formula (I-int-f) upon coupling with an intermediate of B-R³, where B represents a suitable reactive moiety, such as a boronic acid, a boronic acid ester (e.g., a pinacol ester), or a trialkyl tin moiety (e.g., tributyl) in a cross-coupling reaction (e.g., Suzuki, Stille, etc.). In several embodiments, a Suzuki coupling is performed using a palladium or similar transition metal catalyst together with appropriate ligands according to methods described in the literature, and as provided in the Examples below. In several embodiments, the intermediate of Formula (I-Int-f) is further subject to coupling conditions to provide a compound of Formula (A) or Formula (I), as shown in Scheme 2d.

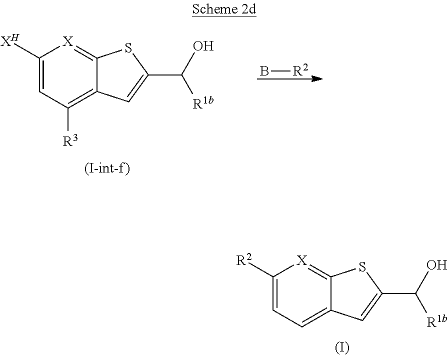

As illustrated in Scheme 2d, compounds of Formula (A) or Formula (I) can be prepared by the reaction of an intermediate of Formula (I-int-f), in which X^{H} represents a suitable halogen atom (e.g., -C₁) or a similar reactive group with an intermediate of B-R², where B represents a suitable reactive moiety, such as a boronic acid, a boronic acid ester (e.g., a pinacol ester), or a trialkyl stannyl group (e.g., tributyl) in a cross-coupling reaction (e.g., Suzuki or Stille). In several embodiments, a Suzuki coupling is performed using a palladium or similar transition metal catalyst together with appropriate ligands according to methods described in the literature, and as provided in the Examples below.

### Scheme 2e

Scheme 2e provides a representative and nonlimiting illustration of the synthesis performed in

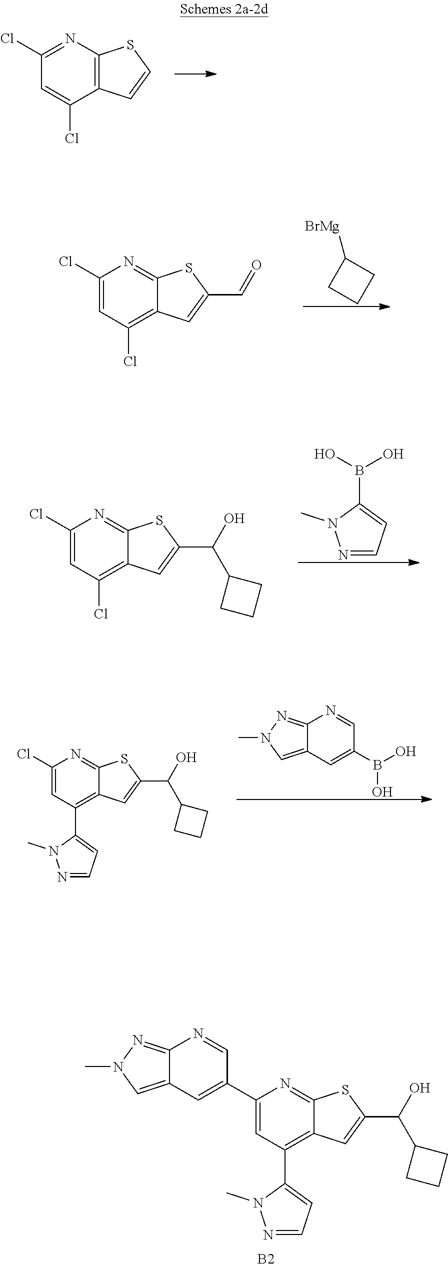

Provided herein as Embodiment 336 is a method of manufacturing the compound of Embodiment 1 or pharmaceutically acceptable salt thereof, the method comprising:
performing a Suzuki coupling between a compound of Formula (1-int)

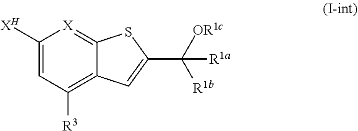

with a compound of Formula (R2-B):

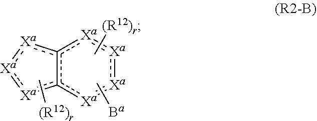

where
X^{H} is an appropriate moiety for Suzuki or Stille coupling;
B^{a} is a boronic acid group or a boronic ester group;
[symbol] indicates a double bond or single bond may be present;
each instance of X^{a} independently is nitrogen, oxygen, or carbon;
each instance of r is an integer selected from 0, 1, 2, 3, 4, or 5, wherein where two instances of r occur on the same R² group, the total of both r values combined does not exceed 5;
each instance of R¹², when present and attached to an N ring member, independently is C₁₋₆alkyl, C₁₋₆haloalkyl, C₁₋₆alkylene-OH, or C₁₋₆alkylene-O—C₁₋₆alkyl; and each instance of R¹², when present and attached to a C ring member, independently is —OH, oxo, C₁₋₆ alkyl, C₁₋₆ haloalkyl, C₁₋₆ alkoxy, C₁₋₆alkylene-OH, or C₁₋₆alkylene-O—C₁₋₆alkyl. In several embodiments, a —N=ring nitrogen can be bonded to an R¹² to provide a cation. In several embodiments, no —N=ring nitrogen is bonded to an R¹² to provide a cation. In several embodiments, X^{H} is a suitable halogen. In several embodiments, X^{H} is —Cl, —Br, or —I. In several embodiments, X^{H} is —Br or —Cl. In several embodiments, X^{H} is another appropriate moiety for Suzuki or Stille coupling. In several embodiments, X^{H} is -OTf, -OTs, or -OMs. In several embodiments, B^{a} is a boronic acid pinacol ester.

Several embodiments provided herein and in the following paragraphs pertain to intermediates. In several embodiments, the variables of these intermediates may be defined as provided below or as defined elsewhere herein. Thus, for example, where R³ (or R^{1a}, R^{1b}, or any other variable) is defined in one manner for Formula (A) or Formula (I) and in another way for Formula (I-int-1), the definition for R³ (or R^{a}, R^{1b}, or any other variable) in Formula (I) may be substituted for the definition of R (or R^{a}, R^{1b} or any other variable) in Formula (I-int-1) and vice versa. Likewise, the definitions for like variables in two different intermediates may be used interchangeably.

Provided herein as Embodiment 337 is a method of manufacturing the compound or salt of Embodiment 1, the method comprising:

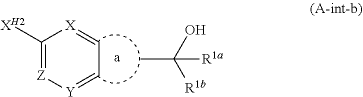

performing a Suzuki coupling between a compound of Formula (A-int-b)

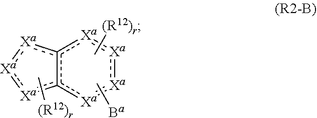

where
X^{H} is a —Br or —Cl;
B^{a} is a boronic acid group or a boronic ester group;
[symbol] indicates a double bond or single bond may be present;
each instance of X^{a} independently is nitrogen, oxygen, or carbon;
each instance of r, where present, is an integer selected from 0, 1, 2, 3, 4, or 5, wherein where two instances of r occur on the same R² group, the total of both r values combined does not exceed 5;
each instance of R¹², when present and attached to an N ring member, independently is C₁₋₆alkyl, C₁₋₆haloalkyl, C₁₋₆alkylene-OH, or C₁₋₆alkylene-O—C₁₋₆alkyl; and
each instance of R¹², when present and attached to a C ring member, independently is —OH, oxo, —N(R^{b})₂, C₁₋₆ alkyl, C₁₋₆ haloalkyl, C₁₋₆ alkoxy, C₁₋₆alkylene-OH, or C₁₋₆alkylene-O—C₁₋₆alkyl;
where each instance of R, where present, independently is —H or C₁₋₆ alkyl; or, alternatively, two R substituents together form a heterocyclyl having 3 to 6 ring members; and
   wherein, when R is C₁₋₆ alkyl or, alternatively, two R substituents together form a heterocyclyl having 3 to 6 ring members, then R^{b} may be unsubstituted or substituted with one or more substituents, each of which independently is —F, —Cl, —OH, C₁₋₃ alkoxy.

Provided herein as Embodiment 338 is a compound or salt of Embodiment 1, made by a method comprising:
performing a Suzuki coupling between a compound of Formula (A-int-b)

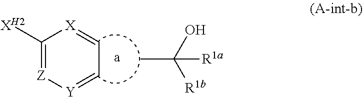

with a compound of Formula (R2-B):

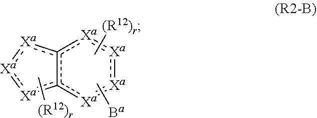

where
X^{H} is a —Br or —Cl;
B^{a} is a boronic acid group or a boronic ester group;
[symbol] indicates a double bond or single bond may be present;
each instance of X^{a} independently is nitrogen, oxygen, or carbon;
each instance of r, where present, is an integer selected from 0, 1, 2, 3, 4, or 5, wherein where two instances of r occur on the same R² group, the total of both r values combined does not exceed 5;
each instance of R¹², when present and attached to an N ring member, independently is C₁₋₆alkyl, C₁₋₆haloalkyl, C₁₋₆alkylene-OH, or C₁₋₆alkylene-O—C₁₋₆alkyl; and
each instance of R¹², when present and attached to a C ring member, independently is —OH, oxo, —N(R^{b})₂, C₁₋₆ alkyl, C₁₋₆ haloalkyl, C₁₋₆ alkoxy, C₁₋₆alkylene-OH, or C₁₋₆alkylene-O—C₁₋₆alkyl;
where each instance of R, where present, independently is —H or C₁₋₆ alkyl; or, alternatively, two R^{b} substituents together form a heterocyclyl having 3 to 6 ring members; and
   wherein, when R^{b} is C₁₋₆ alkyl or, alternatively, two R^{b} substituents together form a heterocyclyl having 3 to 6 ring members, then R² may be unsubstituted or substituted with one or more substituents, each of which independently is —F, —Cl, —OH, C₁₋₃ alkoxy.

Provided herein as Embodiment 339 is a method of manufacturing the compound or salt of Embodiment 1 or 27, the method comprising using a compound of Formula (A-int-a) as an intermediate:

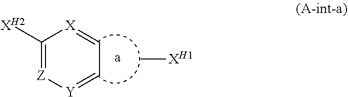

or a pharmaceutically acceptable salt of said compound;
   where
X^{H1} is —Cl, —Br, or —I; and
X^{H2} is —Cl, —Br, -OTf, -OTs, or -OMs.

Provided herein as Embodiment 340 is a method of manufacturing the compound or salt of Embodiment 1 or 27, the method comprising using a compound of Formula (A-int-b.2) as an intermediate:

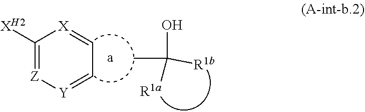

where
X^{H2}—Cl, —Br, —I, -OTf, -OTs, or -OMs; and
R^{1a} and R^{1b} together form a C₃₋₁₀ cycloalkyl or heterocyclyl having 3 to 10 ring members, wherein the R^{1a} and R^{1b} cycle is unsubstituted or substituted.

Provided herein as Embodiment 341 is a compound of Formula (I-int-1):

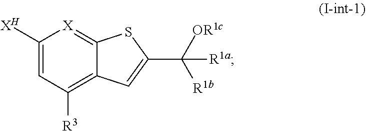

wherein X^{H} is an appropriate moiety for Suzuki or Stille coupling; R^{1a} is —H, C₁₋₆ alkyl, or C₁₋₆ haloalkyl; wherein R^{1a} may be unsubstituted or substituted with one or more substituents, each of which independently is halogen, —OH, —N(R^{b})₂, C₁₋₃ alkyl, or C₁₋₃ alkoxy; R^{1b} is —H, C₁₋₆ alkyl, C₁₋₆ haloalkyl, C₃. 10 cycloalkyl, or heterocyclyl having 3 to 10 ring members; wherein R^{1b} may be unsubstituted or substituted with one or more substituents, each of which independently is halogen, —OH, —N(R^{b})₂, C₁₋₃ alkyl, or C₁₋₃ alkoxy; or, alternatively, R^{1a} and R^{1b} together form a C₃₋₁₀ cycloalkyl, C₃₋₁₀ cycloalkenyl, or heterocyclyl having 3 to 10 ring members; wherein the R^{1a} and R^{1b} cycle may be unsubstituted or substituted with one or more substituents, each of which independently is halogen, —OH, oxo, —N(R^{b})₂, C₁₋₃ alkyl, C₁₋₃alkoxy, or C₁₋₆ heteroalkyl; R^{1c} is —H, C₁₋₆ alkyl, or C₃₋₆ cycloalkyl; wherein R^{1c} may be unsubstituted or substituted with one or more substituents, each of which independently is halogen, —OH, —N(R^{b})₂, C₁₋₃ alkyl, C₁₋₃ alkoxy, or C₁₋₆heteroalkyl; R³ is —H, C₁₋₆ alkyl, C₁₋₆ haloalkyl, C₃₋₆ cycloalkyl, amino, C₁₋₄alkyl-amino, di-C₁₋₄alkyl-amino, C₆₋₁₀ aryl, heteroaryl having 5 to 10 ring members, or heterocyclyl having 5 to 10 ring members; wherein R³ may be unsubstituted or substituted with one or more substituents, each of which independently is halogen, —OH, oxo, —N(R^{b})₂, C₁₋₃ alkyl, C₁₋₃ haloalkyl, C₁₋₃ alkoxy, C₁₋₃ alkylene-C₁₋₃ alkoxy, C₁₋₆ heteroalkyl, or two R³ substituents together form a C₃₋₆ carbocyclyl or a heterocyclyl having 3 to 6 ring members; where each instance of R^{b}, where present, independently is —H or C₁₋₆ alkyl; or, alternatively, two R^{b} substituents together form a heterocyclyl having 3 to 6 ring members; wherein R^{b} may be unsubstituted or substituted with one or more substituents, each of which independently is —F, —Cl, —OH, C₁₋₃ alkoxy. In several embodiments, X^{H} is a suitable halogen. In several embodiments, X^{H} is —Cl, —Br, or —I. In several embodiments, X^{H} is —Br or —Cl. In several embodiments, X^{H} is another appropriate moiety for Suzuki or Stille coupling. In several embodiments, X^{H} is -OTf, -OTs, or -OMs.

Provided herein as Embodiment 342 is a compound of Formula (I-int-a):

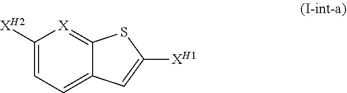

or a pharmaceutically acceptable salt of said compound; where X is N or CH; X^{H1} is —H or halogen; and X^{H2} is an appropriate moiety for Suzuki or Stille coupling. In several embodiments, X^{H}i is a halogen. In several embodiments, X^{H}i is —Cl, —Br, or —I. In several embodiments, X^{H}i is —H. In several embodiments, where it is —H, X^{H}i may be removed and exchanged for a Li atom (e.g., using a lithiating agent such as n-butyllithium). In several embodiments, X^{H2} is a suitable halogen. In several embodiments, X^{H2} is —Br or —Cl. In several embodiments, X^{H2} is another appropriate moiety for Suzuki or Stille coupling. In several embodiments, X^{H2} is -OTf, -OTs, or -OMs (or another sulfonate-based moiety). In several embodiments, X^{H2} is —Br, —Cl, -OTf, -OTs, or -OMs.

Provided herein as Embodiment 343 is a compound of Formula (I-int-b):

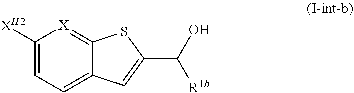

or a pharmaceutically acceptable salt of said compound; where X is N or CH; X^{H2} is an appropriate moiety for Suzuki or Stille coupling; R^{1b} is —H, C₁₋₆ alkyl, C₁₋₆ haloalkyl, C₃₋₁₀ cycloalkyl, or heterocyclyl having 3 to 10 ring members; wherein R^{1b} may be unsubstituted or substituted with one or more substituents, each of which independently is halogen, —OH, —N(R^{b})₂, C₁₋₃ alkyl, or C₁₋₃ alkoxy; where each instance of R^{b}, where present, independently is —H or C₁₋₆ alkyl; or, alternatively, two R^{b} substituents together form a heterocyclyl having 3 to 6 ring members; and wherein R^{b} may be unsubstituted or substituted with one or more substituents, each of which independently is —F, —Cl, —OH, C₁₋₃ alkoxy. In several embodiments, X^{H2} is a suitable halogen. In several embodiments, X^{H2} is —Br or —Cl. In several embodiments, X^{H2} is another appropriate moiety for Suzuki or Stille coupling. In several embodiments, X^{H2} is -OTf, -OTs, or -OMs. In several embodiments, X^{H2} is —Br, —Cl, -OTf, -OTs, or -OMs.

Provided herein as Embodiment 344 is a compound of Formula (I-int-c):

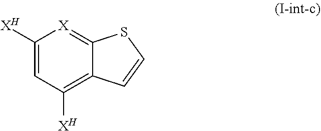

or a pharmaceutically acceptable salt of said compound; where X is N or CH; and each instance of X^{H} is an appropriate moiety for Suzuki or Stille coupling. In several embodiments, each instance of X^{H} is a suitable halogen. In several embodiments, each instance of X^{H} independently is —Br or —Cl. In several embodiments, at least one instance of X^{H} is another appropriate moiety for Suzuki or Stille coupling (e.g., -OTf, -OTs, or -OMs).

Provided herein as Embodiment 345 is a compound of Formula (I-int-d):

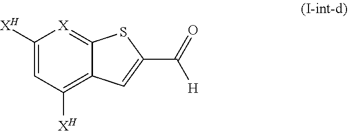

or a pharmaceutically acceptable salt of said compound; where X is N or CH; and each instance of X^{H} is an appropriate moiety for Suzuki or Stille coupling. In several embodiments, each instance of X^{H} is a suitable halogen. In several embodiments, each instance of X^{H} independently is —Br or —Cl. In several embodiments, at least one instance of X^{H} is another appropriate moiety for Suzuki or Stille coupling (e.g., -OTf, -OTs, or -OMs).

Provided herein as Embodiment 346 is a compound of Formula (I-int-e):

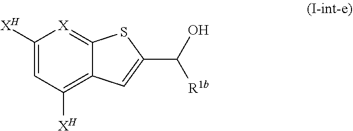

or a pharmaceutically acceptable salt of said compound; where X is N or CH; R^{1b} is —H, C₁₋₆ alkyl, C₁₋₆ haloalkyl, C₃₋₁₀ cycloalkyl, or heterocyclyl having 3 to 10 ring members; wherein R^{1b} may be unsubstituted or substituted with one or more substituents, each of which independently is halogen, —OH, —N(R^{b})₂, C₁₋₃ alkyl, or C₁₋₃ alkoxy; each instance of X^{H} is an appropriate moiety for Suzuki or Stille coupling; where each instance of R^{b}, where present, independently is —H or C₁₋₆ alkyl; or, alternatively, two R^{b} substituents together form a heterocyclyl having 3 to 6 ring members; and wherein R^{b} may be unsubstituted or substituted with one or more substituents, each of which independently is —F, —Cl, —OH, C₁₋₃ alkoxy. In several embodiments, each instance of X^{H} is a suitable halogen. In several embodiments, each instance of X^{H} independently is —Br or —Cl. In several embodiments, at least one instance of X^{H} is another appropriate moiety for Suzuki or Stille coupling (e.g., -OTf, -OTs, or -OMs).

Provided herein as Embodiment 347 is a compound of Formula (I-int-f):

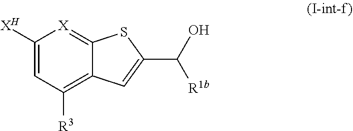

where X is N or CH; R^{1b} is —H, C₁₋₆ alkyl, C₁₋₆ haloalkyl, C₃₋₁₀ cycloalkyl, or heterocyclyl having 3 to 10 ring members; wherein R^{1b} may be unsubstituted or substituted with one or more substituents, each of which independently is halogen, —OH, —N(R^{b})₂, C₁₋₃ alkyl, or C₁₋₃ alkoxy; R³ is —H, C₁₋₆ alkyl, C₁₋₆ haloalkyl, C₃₋₆ cycloalkyl, amino, C₁₋₄alkyl-amino, di-C₁₋₄alkyl-amino, C₆₋₁₀ aryl, heteroaryl having 5 to 10 ring members, or heterocyclyl having 5 to 10 ring members; wherein R³ may be unsubstituted or substituted with one or more substituents, each of which independently is halogen, —OH, oxo, —N(R^{b})₂, C₁₋₃ alkyl, C₁₋₃ haloalkyl, C₁₋₃ alkoxy, C₁₋₃ alkylene-C₁₋₃ alkoxy, C₁₋₆heteroalkyl, or two R³ substituents together form a C₃₋₆ carbocyclyl or a heterocyclyl having 3 to 6 ring members; X^{H} is an appropriate moiety for Suzuki or Stille coupling; where each instance of R^{b}, where present, independently is —H or C₁. ₆ alkyl; or, alternatively, two R^{b} substituents together form a heterocyclyl having 3 to 6 ring members; and wherein R^{b} may be unsubstituted or substituted with one or more substituents, each of which independently is —F, —Cl, —OH, C₁₋₃ alkoxy. In several embodiments, X^{H} is —Cl, —Br, or —I. In several embodiments, X^{H} is —C₁ or —Br. In several embodiments, X^{H} IS -C₁. In several embodiments, X^{H} is —Br.

### Enumerated Embodiments

The following provide exemplary illustrative enumerated embodiments not meant to limit the remainder of the disclosure.
A1. A compound of Formula (I)

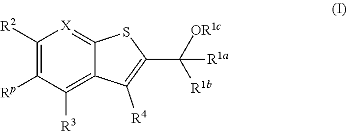

or a pharmaceutically acceptable salt of said compound;
   where
X is N or CH;
R^{1a} is —H, C₁₋₆ alkyl, or C₁₋₆ haloalkyl;
   wherein R^{1a} may be unsubstituted or substituted with one or more substituents, each of which independently is halogen, —OH, —N(R^{b})₂, C₁₋₃ alkyl, or C₁₋₃ alkoxy;
R^{1b} is —H, C₁₋₆ alkyl, C₁₋₆ haloalkyl, C₁₋₆ heteroalkyl, C₃₋₁₀ cycloalkyl, or heterocyclyl having 3 to 10 ring members;
   wherein R^{1b} may be unsubstituted or substituted with one or more substituents, each of which independently is halogen, —OH, —N(R^{b})₂, C₁₋₃ alkyl, or C₁₋₃ alkoxy;
or, alternatively, R^{1a} and R^{1b} together form a C₃₋₁₀ cycloalkyl, C₃₋₁₀ cycloalkenyl, or heterocyclyl having 3 to 10 ring members;
   wherein the R^{1a} and R^{1b} cycle may be unsubstituted or substituted with one or more substituents, each of which independently is halogen, —OH, oxo, —N(R^{b})₂, C₁₋₃ alkyl, C₁₋₃alkoxy, or C₁₋₆ heteroalkyl;
R^{1c} is —H, C₁₋₆ alkyl, or C₃₋₆ cycloalkyl;
   wherein R^{1c} may be unsubstituted or substituted with one or more substituents, each of which independently is halogen, —OH, —N(R^{b})₂, C₁₋₃ alkyl, C₁₋₃ alkoxy, or C₁₋₆heteroalkyl;
R² is C₃₋₂₀ cycloalkyl, C₃₋₂₀ cycloalkenyl, C₈₋₂₀ cycloalkynyl, C₆₋₁₈ aryl, heteroaryl having 5 to 20 ring members, or heterocyclyl having 3 to 20 ring members;
   wherein R² may be unsubstituted or substituted with one or more substituents, each of which independently is halogen, —OH, oxo, —N(R^{b})₂, C₁₋₆ alkyl, C₁₋₆ haloalkyl, C₁₋₆ alkoxy, C₁₋₆ alkylene-OH, C₁₋₆alkylene-O—C₁₋₆alkyl, or C₁₋₆ heteroalkyl;
R³ is —H, C₁₋₆ alkyl, C₁₋₆ haloalkyl, C₃₋₆ cycloalkyl, amino, C₁₋₄alkyl-amino, di-C₁₋₄alkyl-amino, C₆₋₁₀ aryl, heteroaryl having 5 to 10 ring members, or heterocyclyl having 5 to 10 ring members;
   wherein R³ may be unsubstituted or substituted with one or more substituents, each of which independently is halogen, —OH, oxo, —N(R^{b})₂, C₁₋₃ alkyl, C₁₋₃ haloalkyl, C₁₋₃ alkoxy, C₁₋₃ alkylene-C₁₋₃ alkoxy, C₁₋₆ heteroalkyl, or two R³ substituents together form a C₃₋₆ carbocyclyl or a heterocyclyl having 3 to 6 ring members;
R⁴ is —H or —N(R^{a})₂ where each instance of R^{a}, where present, independently is —H or C₁₋₆ alkyl; or, alternatively, two R^{a} substituents together form a heterocyclyl having 3 to 6 ring members;
   wherein R^{a} may be unsubstituted or substituted with one or more substituents, each of which independently is —F, —Cl, —OH, C₁₋₃ alkoxy; RP is —H, C₁₋₆ alkyl, C₃₋₆ cycloalkyl, C₁₋₆ haloalkyl, or C₁₋₆heteroalkyl;
   wherein RP may be unsubstituted or substituted one or more substituents, each of which independently is halogen, —OH, C₁₋₃ alkyl, or C₁₋₃ alkoxy;
where each instance of R^{b}, where present, independently is —H or C₁₋₆ alkyl; or, alternatively, two R^{b} substituents together form a heterocyclyl having 3 to 6 ring members; and
   wherein R^{b} may be unsubstituted or substituted with one or more substituents, each of which independently is —F, —Cl, —OH, C₁₋₃ alkoxy.
A2. The compound or salt of Embodiment A1, wherein the compound of Formula (I) is not:
6-phenyl-4-(trifluoromethyl)thieno[2,3-b]pyridine-2-methanol;
6-cyclopropylbenzo[b]thiophene-2-methanol;
6-phenylbenzo[b]thiophene-2-methanol;
6-[3-[(dimethylamino)methyl]bicyclo[2.2.1]hept-2-en-2-yl]benzo[b]thiophene-2-methanol;
   A3. The compound or salt of Embodiment A1 or A2, wherein R² is not unsubstituted phenyl.
   A4. The compound or salt of Embodiment A1 or A2, wherein when R² is unsubstituted cyclopropyl, unsubstituted phenyl, or unsubstituted or substituted cycloalkenyl, then one of R¹, R^{1b}, and R^{1c} is not —H.
   A5. The compound or salt of any one of Embodiments A1 to A4, wherein X is N.
   A6. The compound or salt of any one of Embodiments A1 to A4, wherein X is CH.
   A7. The compound or salt of any one of Embodiments A1 to A6, wherein R^{1a} is —H, C₁₋₃alkyl, or C₁₋₃haloalkyl.
   A8. The compound or salt of any one of Embodiments A1 to A6, wherein R^{1a} is C₁₋₃alkyl.
   A9. The compound or salt of any one of Embodiments A1 to A6, wherein R^{1a} is C₁₋₃haloalkyl.
   A10. The compound or salt of any one of Embodiments A1 to A9, wherein R^{1a} is substituted with 1, 2, 3, or 4 substituents.
   A11. The compound or salt of any one of Embodiments A1 to A10, wherein R^{1a} is substituted with 1, 2, or 3 substituents.
   A12. The compound or salt of any one of Embodiments A1 to A11, wherein R^{1a} is substituted with 1 or 2 substituents.
   A13. The compound or salt of any one of Embodiments A1 to A12, wherein R^{1a} is substituted with 1 substituent.
   A14. The compound or salt of any one of Embodiments A1 to A13, wherein, when R^{1a} is substituted, each R^{1a} substituent independently is —F, —OH, or C₁₋₃alkoxy.
   A15. The compound or salt of any one of Embodiments A1 to A9, wherein R^{1a} is unsubstituted.
   A16. The compound or salt of any one of Embodiments A1 to A6, wherein R^{1a} is —H, methyl, or CF₃.
   A17. The compound or salt of any one of Embodiments A1 to A6, wherein when R^{1a} is —H.
   A18. The compound or salt of any one of Embodiments A1 to A6, wherein when R^{1a} is —CH₃.
   A19. The compound or salt of any one of Embodiments A1 to A6, wherein when R^{1a} is —CF₃.
   A20. The compound or salt of any one of Embodiments A1 to A19, wherein R^{1b} is —H, C₁₋₃ alkyl, C₁₋₃ haloalkyl, C₃₋₆ cycloalkyl, or heterocyclyl having 3- to 6-ring members.
   A21. The compound or salt of any one of Embodiments A1 to A20, wherein, when R^{1b} is heterocyclyl, the R^{1b} heterocyclyl includes 1, 2, or 3 heteroatom ring members, each of which independently is nitrogen, oxygen, or —S(O)ₓ—, where x is 0, 1, or 2.
   A22. The compound or salt of any one of Embodiments A1 to A21, wherein, when R^{1b} is heterocyclyl, the R^{1b} heterocyclyl includes 1 or 2 heteroatom ring members.
   A23. The compound or salt of any one of Embodiments A1 to A22, wherein, when R^{1b} is heterocyclyl, the R^{1b} heterocyclyl includes 1 heteroatom ring member.
   A24. The compound or salt of any one of Embodiments A1 to A23, wherein, when R^{1b} is heterocyclyl, the heteroatom ring member or ring members of R^{1b} comprise or consist of nitrogen.
   A25. The compound or salt of any one of Embodiments A1 to A24, wherein, when R^{1b} is heterocyclyl, the heteroatom ring member or heteroatom ring members of R^{1b} comprise or consist of oxygen.
   A26. The compound or salt of any one of Embodiments A1 to A20, wherein R^{1b} is C₁₋₃alkyl.
   A27. The compound or salt of any one of Embodiments A1 to A20, wherein R^{1b} is C₁₋₃ haloalkyl.
   A28. The compound or salt of any one of Embodiments A1 to A20, wherein R^{1b} is C₃₋₆cycloalkyl.
   A29. The compound or salt of any one of Embodiments A1 to A25, wherein R^{1b} is 3- to 6-ring member heterocyclyl.
   A30. The compound or salt of any one of Embodiments A1 to A29, wherein R^{1b} is substituted with 1, 2, 3, or 4 substituents.
   A31. The compound or salt of any one of Embodiments A1 to A30, wherein R^{1b} is substituted with 1, 2, or 3 substituents
   A32. The compound or salt of any one of Embodiments A1 to A31, wherein R^{1b} is substituted with 1 or 2 substituents.
   A33. The compound or salt of any one of Embodiments A1 to A32, wherein R^{1b} is substituted with 1 substituent.
   A34. The compound or salt of any one of Embodiments A1 to A33, wherein, when R^{1b} is substituted, each R^{1b} substituent independently is halogen, C₁₋₃alkyl, or C₁₋₃alkoxy.
   A35. The compound or salt of any one of Embodiments A1 to A34, wherein, when R^{1b} is substituted, each R^{1b} substituent independently is —F, —OH, methyl, or —OMe.
   A36. The compound or salt of any one of Embodiments A1 to A19, wherein R^{1b} is C₁₋₆ alkyl substituted with C₁₋₆ alkoxy.
   A37. The compound or salt of Embodiment A36, wherein R^{1b} is C₁₋₃alkyl substituted with C₁₋₃ alkoxy.
   A38. The compound or salt of any one of Embodiments A1 to A29, wherein R^{1b} is unsubstituted.
   A39. The compound or salt of any one of Embodiments A1 to A19, wherein R^{1b} is —H.
   A40. The compound or salt of any one of Embodiments A1 to A6, wherein R^{1a} and R^{1b}together form a C₃₋₆ cycloalkyl or 3 to 6 membered heterocycle.
   A41. The compound or salt of any one of Embodiments A1 to A6 and A40, wherein, when R^{1a} and R^{1b} together form a heterocyclyl, the heterocyclyl includes 1, 2, or 3 heteroatom ring members, each of which independently is nitrogen, oxygen, or —S(O)ₓ—, where x is 0, 1, or 2.
   A42. The compound or salt of any one of Embodiments A1 to A6 and A40-A41, wherein, when R^{1a} and R^{1b} together form a heterocyclyl, the heterocyclyl includes 1 or 2 heteroatom ring members.
   A43. The compound or salt of any one of Embodiments A1 to A6 and A40-A42, wherein, when R^{1a} and R^{1b} together form a heterocyclyl, the heterocyclyl includes 1 heteroatom ring member.
   A44. The compound or salt of any one of Embodiments A1 to A6 and A40-A43, wherein R^{1a} and R^{1b} together form a heterocyclyl the heteroatom ring member or ring members of R^{1a} and R^{1b}comprise or consist of nitrogen.
   A45. The compound or salt of any one of Embodiments A1 to A6 and A40-A44, wherein R^{1a} and R^{1b} together form a heterocyclyl and the heteroatom ring member or heteroatom ring members of R^{1a} and R^{1b} comprise or consist of oxygen.
   A46. The compound or salt of any one of Embodiments A1 to A6 and A40-A45, wherein R^{1a} and R^{1b} together form a cycle that is substituted with 1, 2, 3, or 4 substituents.
   A47. The compound or salt of any one of Embodiments A1 to A6 and A40-A46, wherein R^{1a} and R^{1b} together form a cycle that is substituted with 1, 2, or 3 substituents.
   A48. The compound or salt of any one of Embodiments A1 to A6 and A40-A47, wherein R^{1a} and R^{1b} together form a cycle that is substituted with 1 or 2 substituents.
   A49. The compound or salt of any one of Embodiments A1 to A6 and A40-A47, wherein R^{1a} and R^{1b} together form a cycle that is substituted with 1 substituent.
   A50. The compound or salt of any one of Embodiments A1 to A6 and A40-A49, wherein R^{1a} and R^{1b} together form a cycle that is substituted with C₁₋₃ alkylene-O—C₁₋₃ alkyl as the heteroalkyl.
   A51. The compound or salt of any one of Embodiments A1 to A6 and A40-A49, wherein R^{1a} and R^{1b} together form a cycle that is substituted and each substituent of the cycle independently is —F, —OH, oxo, methyl, or methoxy.
   A52. The compound or salt of any one of Embodiments A1 to A51, wherein R^{1c} is substituted with 1, 2, 3, or 4 substituents.
   A53. The compound or salt of any one of Embodiments A1 to A52, wherein R^{1c} is substituted with 1, 2, or 3 substituents.
   A54. The compound or salt of any one of Embodiments A1 to A53, wherein R^{1c} is substituted with 1 or 2 substituents.
   A55. The compound or salt of any one of Embodiments A1 to A54, wherein R^{1c} is substituted with 1 substituent.
   A56. The compound or salt of any one of Embodiments A1 to A51, wherein R^{1c} is unsubstituted.
   A57. The compound or salt of any one of Embodiments A1 to A51, wherein R^{1c} is —H.
   A58. The compound or salt of any one of Embodiments A1 to A56, wherein R^{1c} is C₁₋₃ alkyl.
   A59. The compound or salt of any one of Embodiments A1 to A51, wherein R^{1c} is methyl.
   A60. The compound or salt of any one of Embodiments A1 to A59, wherein R² is C₃₋₆cycloalkyl, C₆₋₁₀aryl, heteroaryl having 5 to 10 ring members, or heterocyclyl having 5 to 10 ring members.
   A61. The compound or salt of any one of Embodiments A1 to A60, wherein R² is heteroaryl or heterocyclyl and the R²heteroaryl or heterocyclyl includes 1, 2, 3, 4, or 5 heteroatom ring members.
   A62. The compound or salt of any one of Embodiments A1 to A61, wherein R² is heteroaryl or heterocyclyl and the R²heteroaryl or heterocyclyl includes 1, 2, 3, or 4 heteroatom ring members.
   A63. The compound or salt of any one of Embodiments A1 to A62, wherein R² is heteroaryl or heterocyclyl and the R²heteroaryl or heterocyclyl includes 1, 2, or 3 heteroatom ring members.
   A64. The compound or salt of any one of Embodiments A1 to A63, wherein R² is heteroaryl or heterocyclyl and the R₂ heteroaryl or heterocyclyl includes 1 or 2 heteroatom ring members.
   A65. The compound or salt of any one of Embodiments A1 to A64, wherein R² is heteroaryl or heterocyclyl and the R₂ heteroaryl or heterocyclyl includes 1 heteroatom ring member.
   A66. The compound or salt of any one of Embodiments A1 to A65, wherein R² is heteroaryl or heterocyclyl and each ring member heteroatom of the R² ring independently is nitrogen, oxygen, or —S(O)_{y}—, where y is 0, 1, or 2.
   A67. The compound or salt of any one of Embodiments A1 to A65, wherein R² is heteroaryl or heterocyclyl and each heteroatom of the R² ring independently is nitrogen or oxygen.
   A68. The compound or salt of any one of Embodiments A1 to A65, wherein R² is heteroaryl or heterocyclyl and each heteroatom of the R² ring is nitrogen.
   A69. The compound or salt of any one of Embodiments A1 to A60 and A66 to A68, wherein R² is heteroaryl has four N ring atoms.
   A70. The compound or salt of any one of Embodiments A1 to A60 and A66 to A68, wherein the R² heteroaryl has three N ring atoms.
   A71. The compound or salt of any one of Embodiments A1 to A60 and A66 to A68, wherein the R² heteroaryl has two N ring atoms.
   A72. The compound or salt of any one of Embodiments A1 to A60 and A66 to A68, wherein the R² heteroaryl has one N ring atom.
   A73. The compound or salt of any one of Embodiments A1 to A67, wherein R² is heteroaryl or heterocyclyl and each heteroatom of the R² ring is oxygen.
   A74. The compound or salt of any one of Embodiments A1 to A73, wherein R² is fused bicyclic heteroaryl or fused bicyclic heterocyclic.
   A75. The compound or salt of any one of Embodiments A1 to A73, wherein R² is fused bicyclic heteroaryl.
   A76. The compound or salt of any one of Embodiments A1 to A73, wherein R² comprises 5 to 10 ring-members.
   A77. The compound or salt of any one of Embodiments A1 to A73, wherein R² comprises 5 to 9 ring-members.
   A78. The compound or salt of any one of Embodiments A1 to A75, wherein R² is a 9-ring member heteroaryl.
   A79. The compound or salt of any one of Embodiments A1 to A73, wherein R² is a 6-ring member heteroaryl.
   A80. The compound or salt of any one of Embodiments A1 to A73, wherein R² is a 5-ring member heteroaryl.
   A81. The compound or salt of any one of Embodiments A1 to A73, wherein R² is fused bicyclic heterocyclyl.
   A82. The compound or salt of any one of Embodiments A1 to A73, wherein R² is a 9-ring member heterocyclyl.
   A83. The compound or salt of any one of Embodiments A1 to A60 and A64 to A68, wherein R² is a 6-ring member heterocyclyl.
   A84. The compound or salt of any one of Embodiments A1 to A60 and A64 to A68, wherein R² is a 5-ring member heterocyclyl.
   A85. The compound or salt of any one of Embodiments A1 to A59, wherein R² is C₃₋₆cycloalkyl.
   A86. The compound or salt of any one of Embodiments A1 to A59, wherein R² is C₃₋₁₀cycloalkenyl.
   A87. The compound or salt of any one of Embodiments A1 to A59, wherein R² is C₅₋₁₀cycloalkynyl.
   A88. The compound or salt of any one of Embodiments A1 to A59, wherein R² is C₆₋₁₀aryl.
   A89. The compound or salt of any one of Embodiments A1 to A88, wherein R² is substituted with 1, 2, 3, 4, or 5 substituents.
   A90. The compound or salt of any one of Embodiments A1 to A89, wherein R² is substituted with 1, 2, 3, or 4 substituents.
   A91. The compound or salt of any one of Embodiments A1 to A90, wherein R² is substituted with 1, 2, or 3 substituents
   A92. The compound or salt of any one of Embodiments A1 to A91, wherein R² is substituted with 1 or 2 substituents
   A93. The compound or salt of any one of Embodiments A1 to A92, wherein R² is substituted with 1 substituent.
   A94. The compound or salt of any one of Embodiments A1 to A93, wherein, when R² is substituted, each R² substituent independently is halogen, —OH, oxo, C₁₋₃alkyl, or C₁₋₃alkoxy.
   A95. The compound or salt of any one of Embodiments A1 to A93, wherein, when R² is substituted, each R² substituent independently is methyl, ethyl, or isopropyl.
   A96. The compound or salt of any one of Embodiments A1 to A93, wherein, when R² is substituted, R² is substituted with methyl.
   A97. The compound or salt of any one of Embodiments A1 to A93, wherein, when R² is substituted, R² is substituted with ethyl.
   A98. The compound or salt of any one of Embodiments A1 to A93, wherein, when R² is substituted, R² is substituted with isopropyl.
   A99. The compound or salt of any one of Embodiments A1 to A88, wherein R² is unsubstituted.
   A100. The compound or salt of any one of Embodiments A1 to A59, wherein R² is

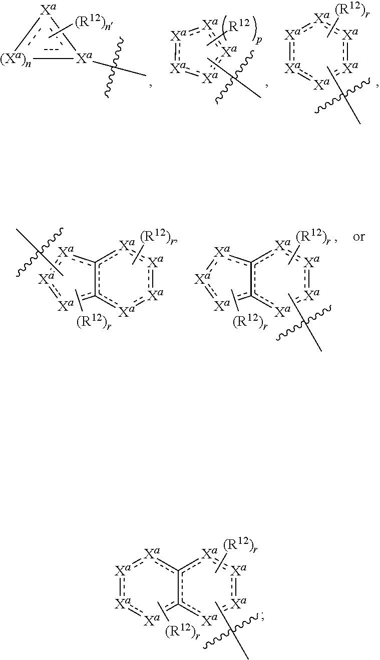

where
[symbol] indicates a double bond or single bond may be present;
each instance of X^{a} independently is nitrogen, oxygen, or carbon;
each instance of n, where present, is an integer selected from 1, 2, 3, 4, or 5;
each instance of n′, where present, is an integer selected from 1, 2, 3, 4, or 5;
each instance of p, where present, is an integer selected from 0, 1, 2, 3, or 4;
each instance of r, where present, is an integer selected from 0, 1, 2, 3, 4, or 5, wherein where two instances of r occur on the same R² group, the total of both r values combined does not exceed 5;
each instance of R¹², when present and attached to an N ring member, is C₁₋₆alkyl, C₁₋₆haloalkyl, C₁₋₆alkylene-OH, or C₁₋₆alkylene-O—C₁₋₆alkyl; and
each instance of R¹², when present and attached to a C ring member, independently is halogen, —OH, oxo, —N(R^{b})₂, C₁₋₆ alkyl, C₁₋₆ haloalkyl, C₁₋₆ alkoxy, C₁₋₆alkylene-OH, or C₁₋₆alkylene-O—C₁₋₆alkyl;
wherein a —N=ring nitrogen can be bonded to an R¹² to provide a cation.
A101. The compound or salt of any one of Embodiments A l to A59, wherein Formula (I) is further represented by the compound of Formula (I2i):

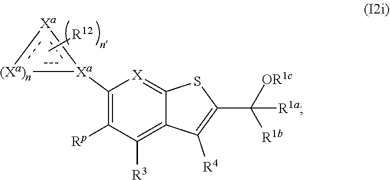

where
[symbol] indicates a double bond or single bond may be present;
each instance of X^{a} independently is nitrogen, oxygen, or carbon;
each instance of n, where present, is an integer selected from 1, 2, 3, 4, or 5; and
each instance of n′, where present, is an integer selected from 1, 2, 3, 4, or 5;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, oxo, C₁₋₃alkyl, C₁₋₃haloalkyl, C₁₋₃alkylene-OH, or C₁₋₃alkylene-O—C₁₋₃alkyl;
wherein a —N=ring nitrogen can be bonded to an R¹² to provide a cation.
A102. The compound or salt of any one of Embodiments A l to A59, wherein Formula (I) is further represented by the compound of Formula (I2ii):

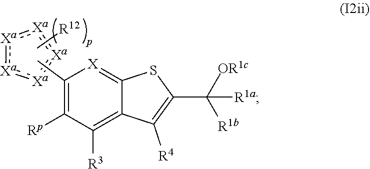

where [symbol]
[symbol] indicates a double bond or single bond may be present;
each instance of X^{a} independently is nitrogen, oxygen, or carbon;
each instance of p, where present, is an integer selected from 0, 1, 2, 3, or 4;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, oxo, C₁₋₃alkyl, C₁₋₃haloalkyl, C₁₋₃alkylene-OH, or C₁₋₃alkylene-O—C₁₋₃alkyl;
wherein a —N=ring nitrogen can be bonded to an R¹² to provide a cation.
A103. The compound or salt of any one of Embodiments A1 to A59, wherein Formula (I) is further represented by the compound of Formula (I2iii):

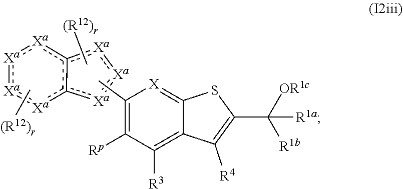

where
[symbol] indicates a double bond or single bond may be present;
each instance of X^{a} independently is nitrogen, oxygen, or carbon;
each instance of r, where present, is an integer selected from 0, 1, 2, 3, 4, or 5, wherein where two instances of r occur on the same R² group, the total of both r values combined does not exceed 5;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, oxo, C₁₋₃alkyl, C₁₋₃haloalkyl, C₁₋₃alkylene-OH, or C₁₋₃alkylene-O—C₁₋₃alkyl;
wherein a —N=ring nitrogen can be bonded to an R¹² to provide a cation.
A104. The compound or salt of any one of Embodiments A l to A59, wherein Formula (I) is further represented by the compound of Formula (I2iv):

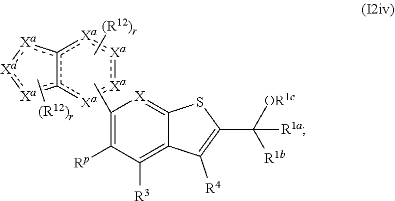

where
[symbol] indicates a double bond or single bond may be present;
each instance of X^{a} independently is nitrogen, oxygen, or carbon;
each instance of r, where present, is an integer selected from 0, 1, 2, 3, 4, or 5, wherein where two instances of r occur on the same R² group, the total of both r values combined does not exceed 5;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, oxo, C₁₋₃alkyl, C₁₋₃haloalkyl, C₁₋₃alkylene-OH, or C₁₋₃alkylene-O—C₁₋₃alkyl;
wherein a —N=ring nitrogen can be bonded to an R¹² to provide a cation.
A105. The compound or salt of any one of Embodiments A l to A59, wherein Formula (I) is further represented by the compound of Formula (I2v):

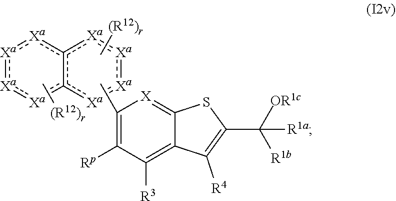

where
[symbol] indicates a double bond or single bond may be present;
each instance of X^{a} independently is nitrogen, oxygen, or carbon;
each instance of r, where present, is an integer selected from 0, 1, 2, 3, 4, or 5, wherein where two instances of r occur on the same R² group, the total of both r values combined does not exceed 5;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, oxo, C₁₋₃alkyl, C₁₋₃haloalkyl, C₁₋₃alkylene-OH, or C₁₋₃alkylene-O—C₁₋₃alkyl;
wherein a —N=ring nitrogen can be bonded to an R¹² to provide a cation.
A106. The compound or salt of any one of Embodiments A1 to A59 and A100, wherein R² is

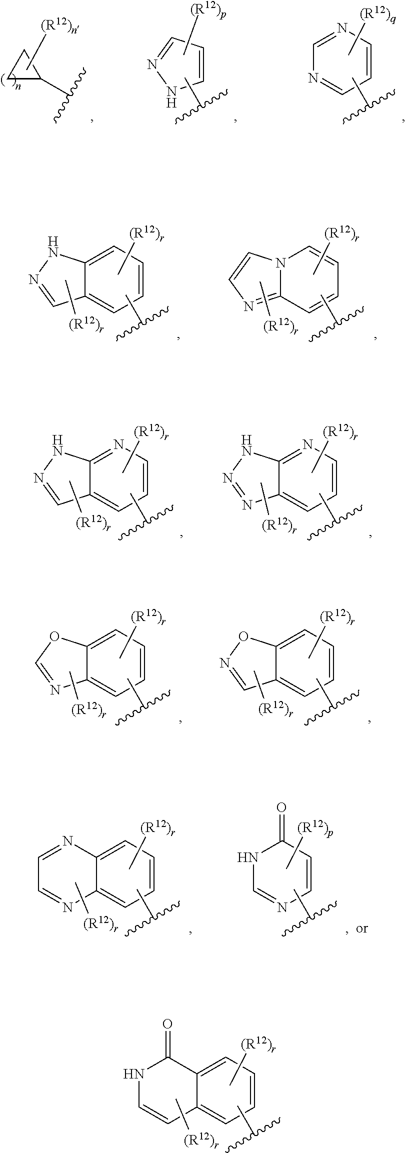

where
each instance of n, where present, is an integer selected from 1, 2, 3, 4, or 5;
each instance of n′, where present, is an integer selected from 0, 1, 2, 3, 4, or 5;
each instance of p, where present, is an integer selected from 0, 1, 2, 3, or 4;
each instance of q, where present, is an integer selected from 0, 1, 2, 3, 4, or 5;
each instance of r, where present, is an integer selected from 0, 1, 2, 3, 4, or 5, wherein where two instances of r occur on the same R² group, the total of both r values combined does not exceed 5;
each instance of s, where present, is an integer selected from 0, 1, or 2;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl;
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl;
wherein an —H on any ring member can be replaced with R¹²;
wherein a —N=ring nitrogen can be bonded to an R¹² to provide a cation.
A107. The compound or salt of any one of Embodiments A1 to A59, wherein R² is:

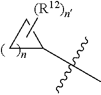

where
n is an integer selected from 1, 2, 3, 4, or 5;
n′ is an integer selected from 0, 1, 2, 3, 4, or 5;
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl.
A108. The compound or salt of an one of Embodiments A1 to A59, wherein R² is: (R¹²)

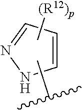

where
p is an integer selected from 0, 1, 2, or 3;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl.
A109. The compound or salt of any one of Embodiments A1 to A59, wherein R² is:

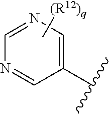

where
q is an integer selected from 0, 1, 2, or 3;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl;
wherein R¹² may be connected to a=N— ring member to provide a cation.
A110. The compound or salt of any one of Embodiments A1 to A59, wherein R² is:

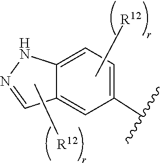

where
each instance of r is an integer selected from 0, 1, 2, or 3;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl;
wherein R¹² may be connected to a=N— ring member to provide a cation.
A111. The compound or salt of any one of Embodiments A1 to A59, wherein R² is:

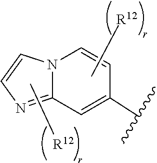

where
each instance of r is an integer selected from 0, 1, 2, or 3;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl;
wherein R¹² may be connected to a=N— ring member to provide a cation.
A112. The compound or salt of any one of Embodiments A1 to A59, wherein R² is:

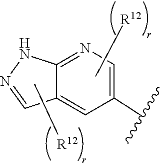

where
each instance of r is an integer selected from 0, 1, 2, or 3;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl;
wherein R¹² may be connected to a=N— ring member to provide a cation.
A113. The compound or salt of any one of Embodiments A1 to A59, wherein R² is:

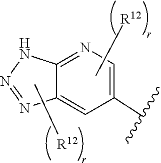

where
each instance of r is an integer selected from 0, 1, 2, or 3;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl;
wherein R¹² may be connected to a=N— ring member to provide a cation.
A114. The compound or salt of any one of Embodiments A1 to A59, wherein R² is:

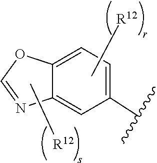

where
each instance of r, where present, is an integer selected from 0, 1, 2, or 3;
each instance of s, where present, is an integer selected from 0, 1, or 2;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl;
wherein R¹² may be connected to a=N— ring member to provide a cation.
A115. The compound or salt of—any one of Embodiments A1 to A59, wherein R² is:

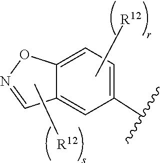

where
each instance of r, where present, is an integer selected from 0, 1, 2, or 3;
each instance of s, where present, is an integer selected from 0, 1, or 2;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl;
wherein R¹² may be connected to a=N— ring member to provide a cation.
A116. The compound or salt of any one of Embodiments A1 to A59, wherein R² is:

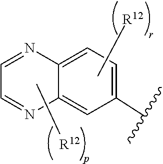

where
each instance of p, where present, is an integer selected from 0, 1, 2, 3, or 4;
each instance of r, where present, is an integer selected from 0, 1, 2, or 3;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl;
wherein R¹² may be connected to a=N— ring member to provide an ammonium cation.
A117. The compound or salt of any one of Embodiments A1 to A59, wherein R² is:

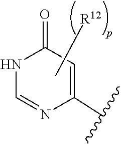

where
each instance of p, where present, is an integer selected from 0, 1, 2, 3, or 4;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl;
wherein R¹² may be connected to a=N— ring member to provide an ammonium cation.
A118. The compound or salt of any one of Embodiments A1 to A59, wherein R² is:

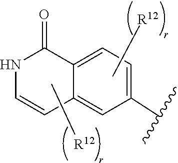

where
each instance of r, where present, is an integer selected from 0, 1, 2, or 3;
each instance of R¹², when present and attached to an N ring member, independently is C₁₋₃alkyl, C₁₋₃haloalkyl, C₁₋₃alkylene-OH, or C₁₋₃alkylene-O—C₁₋₃alkyl; and
each instance of R¹², when present and attached to a C ring member, independently is halogen, —OH, C₁₋₃alkyl, C₁₋₃haloalkyl, C₁₋₃alkylene-OH, or C₁₋₃alkylene-O—C₁₋₃alkyl.
A119. The compound or salt of any one of Embodiments A1 to A59, wherein R² is

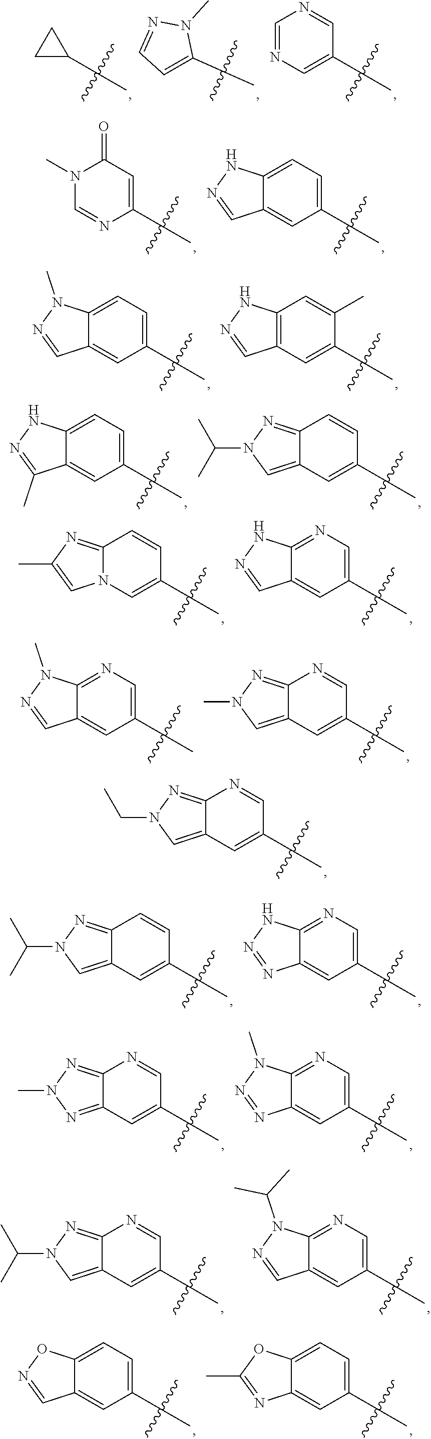

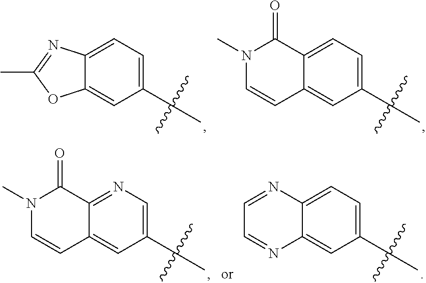 [SMILES: *c1ccc2nc(C)oc2c1 ; *c1ccc2c(=O)n(C)ccc2c1 ; *c1cnc2c(=O)n(C)ccc2c1 ; *c1ccc2nccnc2c1]

A120. The compound or salt of any one of Embodiments A1 to A119, wherein R³ is C₃₋₆cycloalkyl, C₃₋₁₀cycloalkenyl, C₇₋₁₀cycloalkynyl, C₆₋₁₀aryl, heteroaryl having 5 to 10 ring members, or heterocyclyl having 5 to 10 ring members.
A121. The compound or salt of any one of Embodiments A1 to A120, wherein R³ is heteroaryl or heterocyclyl and the R³ heteroaryl or heterocyclyl includes 1, 2, 3, 4, or 5 heteroatom ring members.
A122. The compound or salt of any one of Embodiments A1 to A121, wherein R³ is heteroaryl or heterocyclyl and the R³ heteroaryl or heterocyclyl includes 1, 2, 3, or 4 heteroatom ring members.
A123. The compound or salt of any one of Embodiments A1 to A122, wherein R³ is heteroaryl or heterocyclyl and the R³ heteroaryl or heterocyclyl includes 1, 2, or 3 heteroatom ring members.

A 124. The compound or salt of any one of Embodiments A l to A123, wherein R³ is heteroaryl or heterocyclyl and the R³ heteroaryl or heterocyclyl includes 1 or 2 heteroatom ring members.
A125. The compound or salt of any one of Embodiments A1 to A124, wherein R³ is heteroaryl or heterocyclyl and the R³ heteroaryl or heterocyclyl includes 1 heteroatom ring member.

A 126. The compound or salt of any one of Embodiments A l to A125, wherein R³ is heteroaryl or heterocyclyl and each heteroatom of the R³ ring independently is nitrogen, oxygen, or —S(O)_{z}, where z is 0, 1, or 2.

A 127. The compound or salt of any one of Embodiments A l to A125, wherein R³ is heteroaryl or heterocyclyl and each heteroatom of the R³ ring independently is nitrogen or oxygen.
A128. The compound or salt of any one of Embodiments A1 to A127, wherein R³ is heteroaryl or heterocyclyl and each heteroatom of the R³ ring is nitrogen.
A129. The compound or salt of any one of Embodiments A1 to A 119 and A126 to A128, wherein R³ is heteroaryl has four N heteroatom ring atoms.
A130. The compound or salt of any one of Embodiments A1 to A 119 and A126 to A128, wherein the R³ heteroaryl has three N heteroatoms.
A131. The compound or salt of any one of Embodiments A l to A 119 and A126 to A128, wherein the R³ heteroaryl has two N heteroatoms.
A132. The compound or salt of any one of Embodiments A l to A 119 and A126 to A128, wherein the R³ heteroaryl has one N heteroatom.
A133. The compound or salt of any one of Embodiments A1 to A127, wherein R³ is heteroaryl or heterocyclyl and each heteroatom of the R³ ring is oxygen.
A134. The compound or salt of any one of Embodiments A1 to A133, wherein R³ is fused bicyclic heteroaryl or fused bicyclic heterocyclic.
A135. The compound or salt of any one of Embodiments A1 to A133, wherein R³ is fused bicyclic heteroaryl.
A136. The compound or salt of any one of Embodiments A1 to A133, wherein R³ comprises 5 to 10 ring-members.
A137. The compound or salt of any one of Embodiments A1 to A133, wherein R³ comprises 5 to 9 ring-members.
A138. The compound or salt of any one of Embodiments A1 to A135, wherein R³ is a 9-ring member heteroaryl.
A139. The compound or salt of any one of Embodiments A1 to A133, wherein R³ is a 6-ring member heteroaryl.
A140. The compound or salt of any one of Embodiments A1 to A133, wherein R³ is a 5-ring member heteroaryl.
A141. The compound or salt of any one of Embodiments A1 to A133, wherein R³ is fused bicyclic heterocyclyl.
A142. The compound or salt of any one of Embodiments A1 to A133, wherein R³ is a 9-ring member heterocyclyl.
A143. The compound or salt of any one of Embodiments A1 to A120 and A123 to A127, wherein R³ is a 6-ring member heterocyclyl.
A144. The compound or salt of any one of Embodiments A1 to A120 and A124 to A127, wherein R³ is a 5-ring member heterocyclyl.
A145. The compound or salt of any one of Embodiments A1 to A119, wherein R³ is C₃₋₄cycloalkyl.
A146. The compound or salt of any one of Embodiments A1 to A119, wherein R³ is C₁₋₆ alkyl.

A 147. The compound or salt of any one of Embodiments A l to A 119, wherein R³ is C₆₋₁₀aryl.
A148. The compound or salt of any one of Embodiments A1 to A147, wherein R³ is substituted with 1, 2, 3, 4, or 5 substituents.
A149. The compound or salt of any one of Embodiments A1 to A148, wherein R³ is substituted with 1, 2, 3, or 4 substituents.
A150. The compound or salt of any one of Embodiments A1 to A149, wherein R³ is substituted with 1, 2, or 3 substituents.
A151. The compound or salt of any one of Embodiments A1 to A150, wherein R³ is substituted with 1 or 2 substituents.
A152. The compound or salt of any one of Embodiments A1 to A151, wherein R³ is substituted with 1 substituent.
A153. The compound or salt of any one of Embodiments A1 to A152, wherein, when R³ is substituted, each R³ substituent independently is C₁₋₃alkyl, C₁₋₃alkoxy, or C₁₋₃ alkylene-C₁₋₃ alkoxy.

A 154. The compound or salt of any one of Embodiments A l to A147, wherein, when R³ is substituted, each R³ substituent independently is methyl, ethyl, or isopropyl.
A155. The compound or salt of any one of Embodiments A1 to A154, wherein, when R³ is substituted, R³ is substituted with methyl.
A156. The compound or salt of any one of Embodiments A1 to A154, wherein, when R³ is substituted, R³ is substituted with ethyl.
A157. The compound or salt of any one of Embodiments A1 to A154, wherein, when R³ is substituted, R³ is substituted with isopropyl.
A158. The compound or salt of any one of Embodiments A1 to A147, wherein R³ is unsubstituted.
A159. The compound or salt of any one of Embodiments A1 to A119, wherein R³ is —H.
A160. The compound or salt of any one of Embodiments A1 to A 119, wherein R³ is

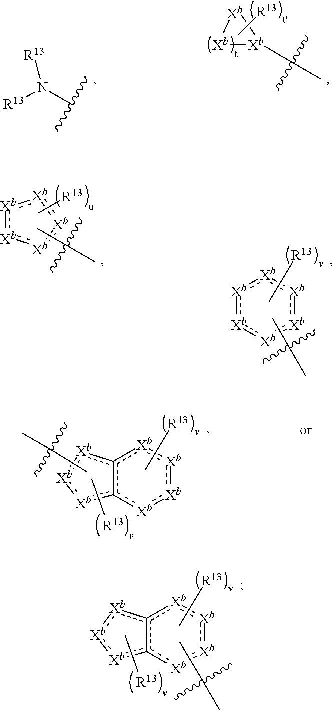

where
[symbol] indicates a double bond or single bond may be present;
each instance of X^{b} independently is nitrogen, oxygen, or carbon;
each instance of t, where present, is an integer selected from 1, 2, 3, 4, or 5;
each instance of t′, where present, is an integer selected from 1, 2, 3, 4, or 5;
each instance of v, where present, is an integer selected from 0, 1, 2, 3, 4, or 5, wherein where two instances of v occur on the same R³ group, the total of both v values combined does not exceed 5;
each instance of R¹³, when present and attached to an N ring member, independently is C₁₋₃alkyl, C₁₋₃haloalkyl, or C₁₋₃ alkylene-C₁₋₃ alkoxy; and
each instance of R¹³, when present and attached to a C ring member, independently is halogen, —OH, oxo, —N(R^{b})₂, C₁₋₃alkyl, C₁₋₃haloalkyl, or C₁₋₃alkylene-O—C₁₋₃alkyl; or, alternatively, two instances of R¹³ together form a C₃₋₆ cycloalkyl or heterocyclyl having 3 to 6 ring members, which may provided a fused ring system or spiro ring system when R³ is a ring structure;
wherein a —N=ring nitrogen can be bonded to an R¹³ to provide a cation.
A161. The compound or salt of any one of Embodiments A1 to A119 wherein R³ is:

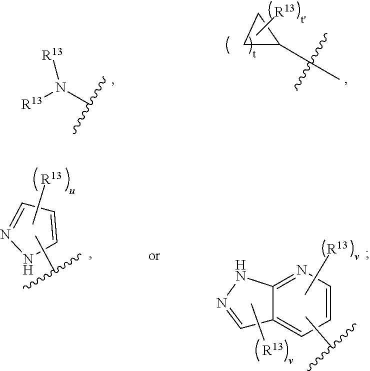

where
each instance of t, where present, is an integer selected from 1, 2, 3, 4, or 5;
each instance of t′, where present, is an integer selected from 1, 2, 3, 4, or 5;
each instance of v, where present, is an integer selected from 0, 1, 2, 3, 4, or 5, wherein where two instances of v occur on the same R³ group, the total of both v values combined does not exceed 5;
each instance of R¹³, when present and attached to an N ring member, independently is C₁₋₃alkyl, C₁₋₃haloalkyl, or C₁₋₃ alkylene-C₁₋₃ alkoxy; and
each instance of R¹³, when present and attached to a C ring member, independently is halogen, —OH, oxo, C₁₋₃alkyl, C₁₋₃haloalkyl, or C₁₋₃alkylene-O-C₁₋₃alkyl;
or, alternatively, two instances of R¹³ together form a C₃₋₆ cycloalkyl or heterocyclyl having 3 to 6 ring members, which may provide a fused ring system or spiro ring system when R³ is a ring structure;
wherein a —N=ring nitrogen can be bonded to an R¹³ to provide a cation.
A162. The compound or salt of any one of Embodiments A1 to A 119, wherein R³ together with its substituents is

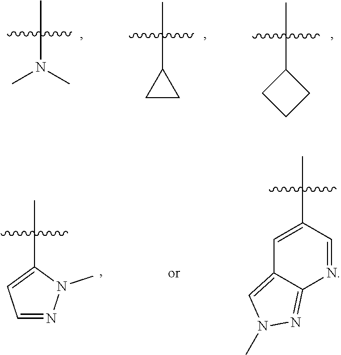

A163. The compound or salt of any one of Embodiments A1 to A 119, wherein R³ is:

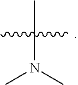

A164. The compound or salt of any one of Embodiments A1 to A119, wherein R³ is:

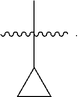 [SMILES: *C1CC1]

A165. The compound or salt of any one of Embodiments A1 to A 119, wherein R³ is:

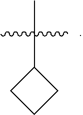 [SMILES: *C1CCC1]

A166. The compound or salt of any one of Embodiments A1 to A119, wherein R³ is:

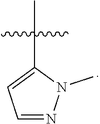 [SMILES: *c1ccnn1C]

A167. The compound or salt of any one of Embodiments A1 to A 119, wherein R³ is:

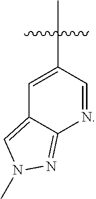 [SMILES: *c1cnc2nn(C)cc2c1]

A168. The compound or salt of any one of Embodiments A1 to A167, wherein each instance of R independently is substituted with 1, 2, 3, 4, or 5 substituents.
A169. The compound or salt of any one of Embodiments A1 to A168, wherein each instance of R independently is substituted with 1, 2, 3, or 4 substituents.
A170. The compound or salt of any one of Embodiments A1 to A169, wherein each instance of R independently is substituted with 1, 2, or 3 substituents.
A171. The compound or salt of any one of Embodiments A l to A170, wherein each instance of R independently is substituted with 1 or 2 substituents.
A172. The compound or salt of any one of Embodiments A1 to A171, wherein each instance of R independently is substituted with 1 substituent.

A 173. The compound or salt of any one of Embodiments A l to A180, wherein each instance of R is unsubstituted.
A174. The compound or salt of any one of Embodiments A1 to A167, wherein R⁴ is —N(R^{a})₂.

A 175. The compound or salt of any one of Embodiments A l to A174, wherein each instance of R independently is substituted with 1, 2, 3, 4, or 5 substituents.
A176. The compound or salt of any one of Embodiments A1 to A175, wherein each instance of R independently is substituted with 1, 2, 3, or 4 substituents.

A 177. The compound or salt of any one of Embodiments A l to A176, wherein each instance of R independently is substituted with 1, 2, or 3 substituents.
A178. The compound or salt of any one of Embodiments A1 to A177, wherein each instance of R independently is substituted with 1 or 2 substituents.
A179. The compound or salt of any one of Embodiments A1 to A178, wherein each instance of R independently is substituted with 1 substituent.
A180. The compound or salt of any one of Embodiments A1 to A167, wherein each instance of R is unsubstituted.
A181. The compound or salt of any one of Embodiments A1 to A180, wherein R^{a} is C₁₋₃ alkyl.
A182. The compound or salt of any one of Embodiments A1 to A174, wherein R^{a} is —CH₃
A183. The compound or salt of any one of Embodiments A1 to A174, wherein R^{a} is —H.
A184. The compound or salt of any one of Embodiments A1 to A167, wherein R⁴ is —H.
A185. The compound or salt of any one of Embodiments A1 to A167, wherein R⁴ is C₁₋₃ alkyl.
A186. The compound or salt of any one of Embodiments A1 to A183, wherein RP independently is substituted with 1, 2, 3, or 4 substituents.
A187. The compound or salt of any one of Embodiments A1 to A186, wherein RP independently is substituted with 1, 2, or 3 substituents.
A188. The compound or salt of any one of Embodiments A1 to A187, wherein RP independently is substituted with 1 or 2 substituents.
A189. The compound or salt of any one of Embodiments A1 to A188, wherein RP independently is substituted with 1 substituent.
A190. The compound or salt of any one of Embodiments A1 to A184, wherein RP is unsubstituted.
A191. The compound or salt of any one of Embodiments A1 to A184, wherein RP is —H, C₁₋₆ alkyl, or C₁₋₆heteroalkyl.

A 192. The compound or salt of any one of Embodiments A l to A184, wherein RP is —H or —CH₂OH.
A193. The compound or salt of any one of Embodiments A1 to A184, wherein R is —H.
A194. The compound or salt of any one of Embodiments A1 to A184, wherein RP is —CH₂OCH₃.
A195. The compound or salt of any one of Embodiments A1 to A51, A57, A60 to A173, and A186 to A194, wherein the compound of Formula (I) is further represented by the compound of Formula (IAi):

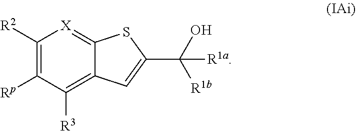

A196. The compound or salt of any one of Embodiments A1 to A51, A57, A60 to A173, and A186 to A194, wherein the compound of Formula (I) is further represented by the compound of Formula (IAii):

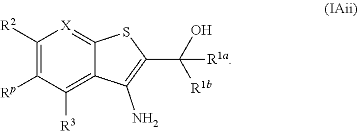

A197. The compound or salt of any one of Embodiments A1 to A51, A57, and A60 to A184, wherein the compound of Formula (I) is further represented by the compound of Formula (IBi):

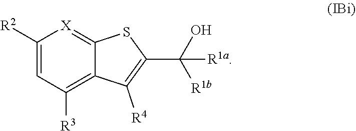

A198. The compound or salt of any one of Embodiments A1 to A51, A57, and A60 to A173, wherein the compound of Formula (I) is further represented by the compound of Formula (IBii):

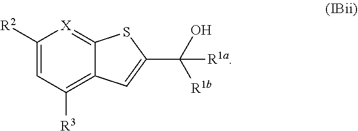

A199. The compound or salt of any one of Embodiments A1 to A51, A57, and A60 to A173, wherein the compound of Formula (I) is further represented by the compound of Formula (IBiii):

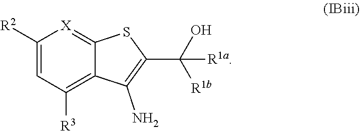

A200. The compound or salt of any one of Embodiments A1 to A51, A57, A60 to A119, and A168 to A194, wherein the compound of Formula (I) is further represented by the compound of Formula (ICi):

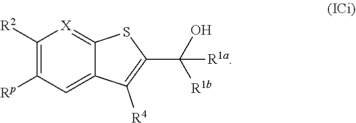

A201. The compound or salt of any one of Embodiments A1 to A51, A57, A60 to A119, A168 to A173, and A186 to A194, wherein the compound of Formula (I) is further represented by the compound of Formula (ICii):

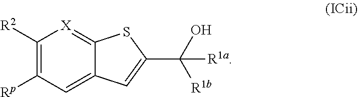

A202. The compound or salt of any one of Embodiments A1 to A51, A57, A60 to A119, A168 to A173, and A186 to A194, wherein the compound of Formula (I) is further represented by the compound of Formula (ICiii):

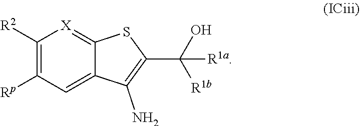

A203. The compound or salt of any one of Embodiments A1 to A51, A57, A60 to A119, and A168 to A184, wherein the compound of Formula (I) is further represented by the compound of Formula (IDi):

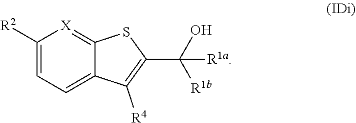

A204. The compound or salt of any one of Embodiments A1 to A51, A57, A60 to A119, and A168 to A173, wherein the compound of Formula (I) is further represented by the compound of Formula (IDii):

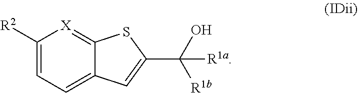

A205. The compound or salt of Embodiment A1, wherein the compound is:

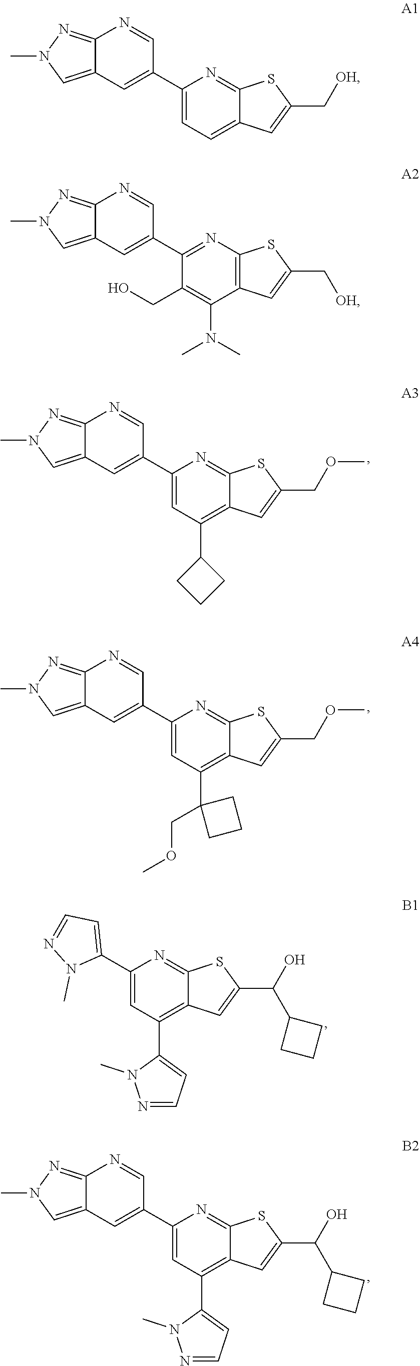

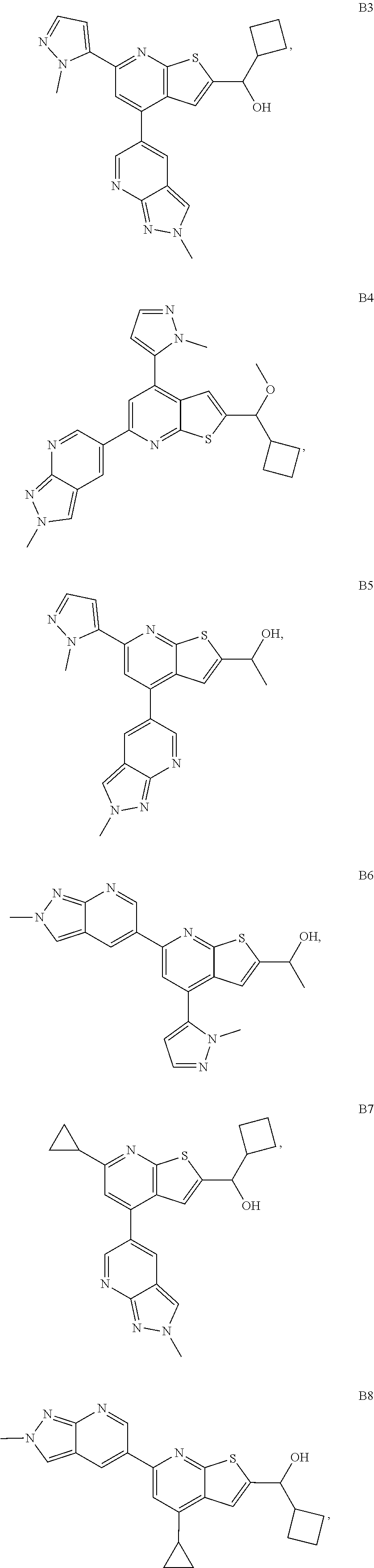

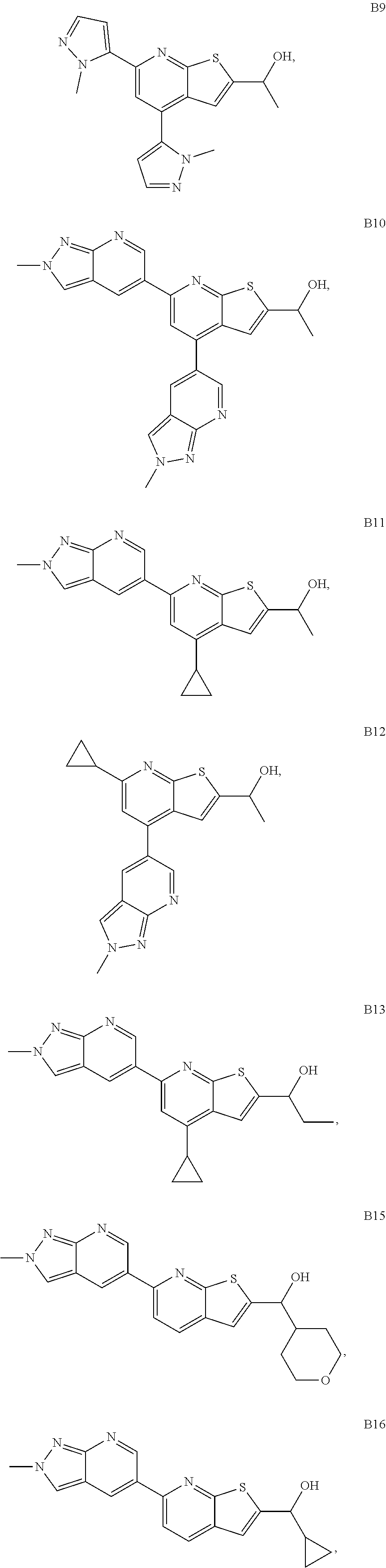

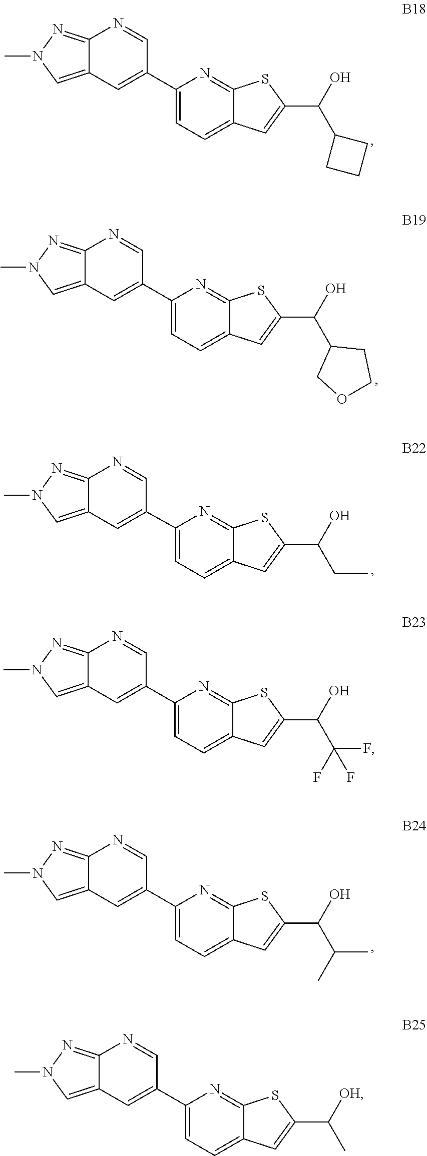

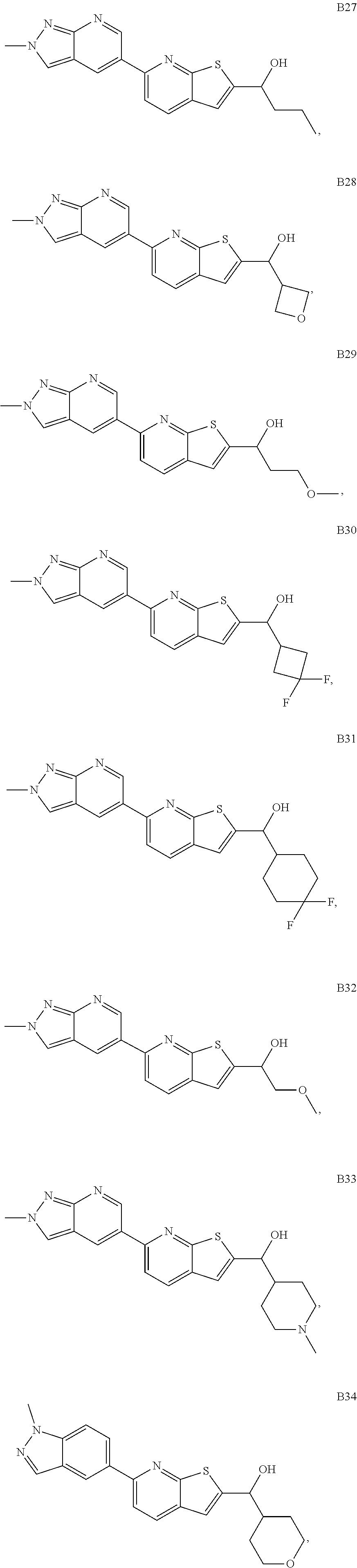

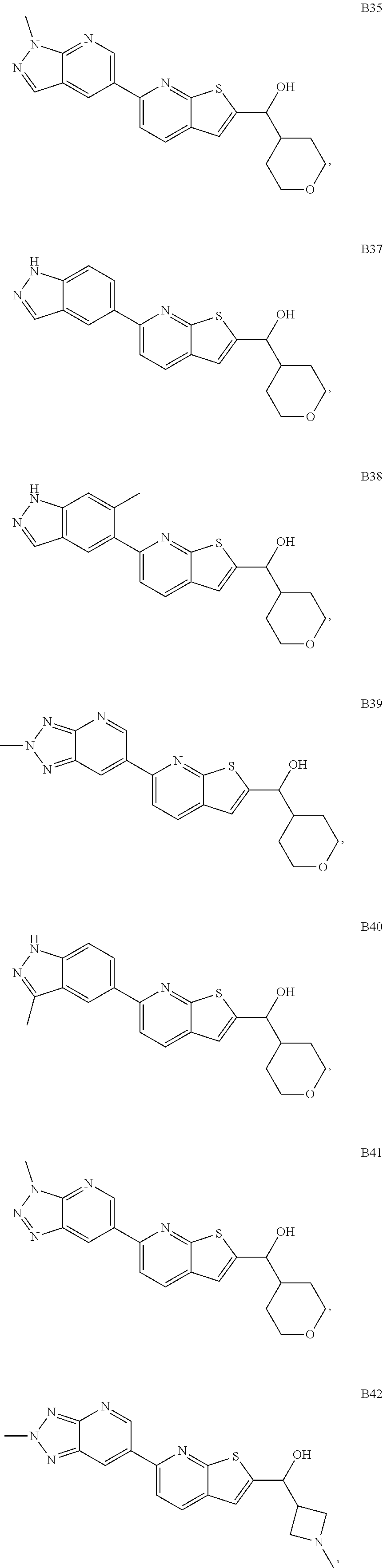

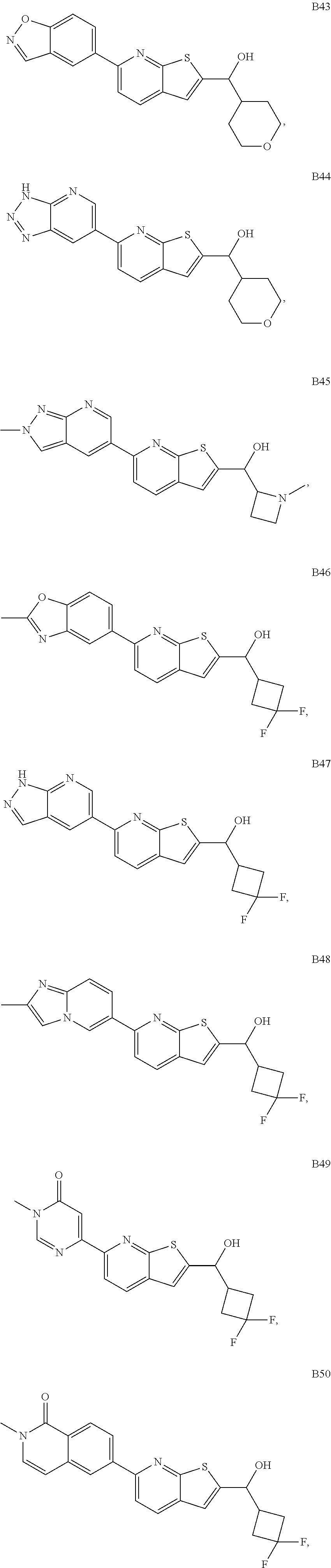

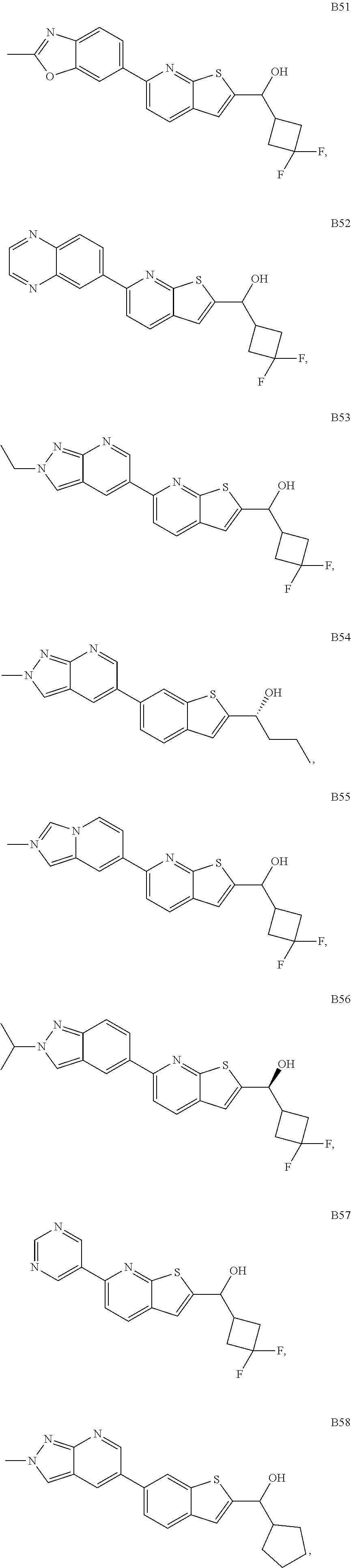

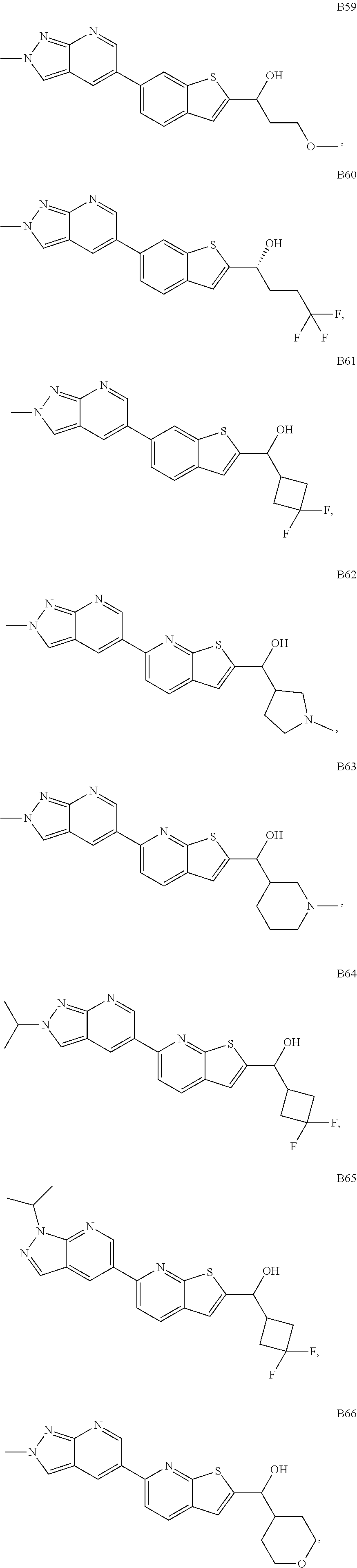

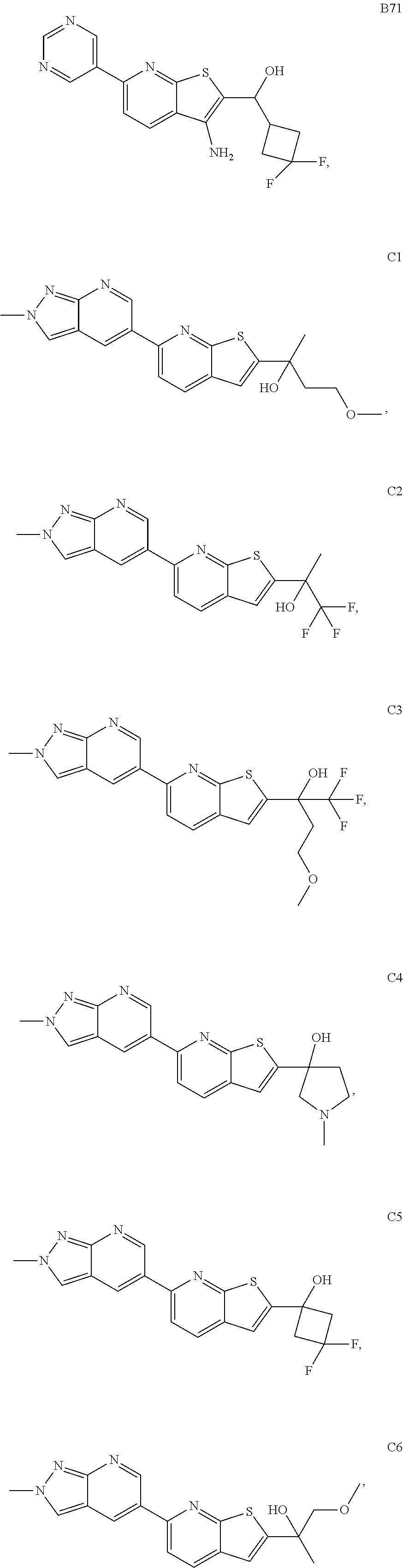

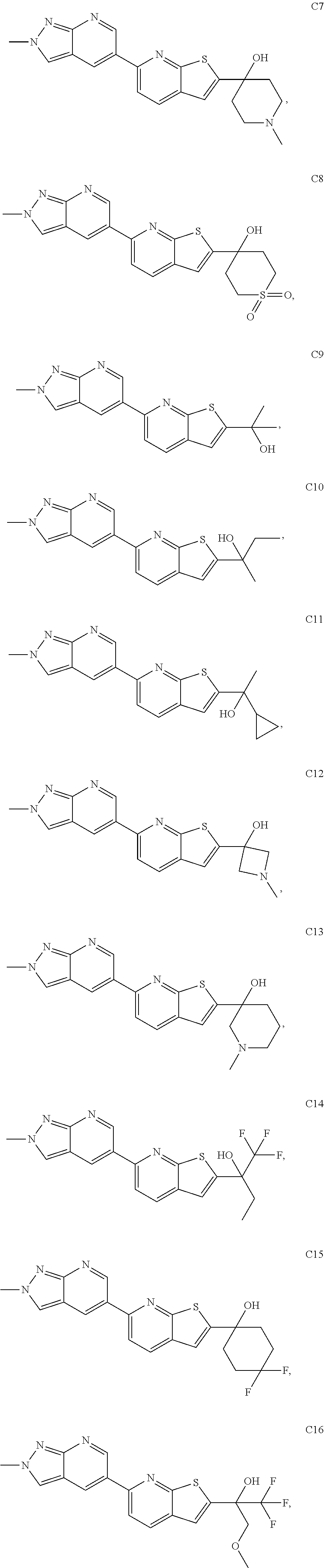

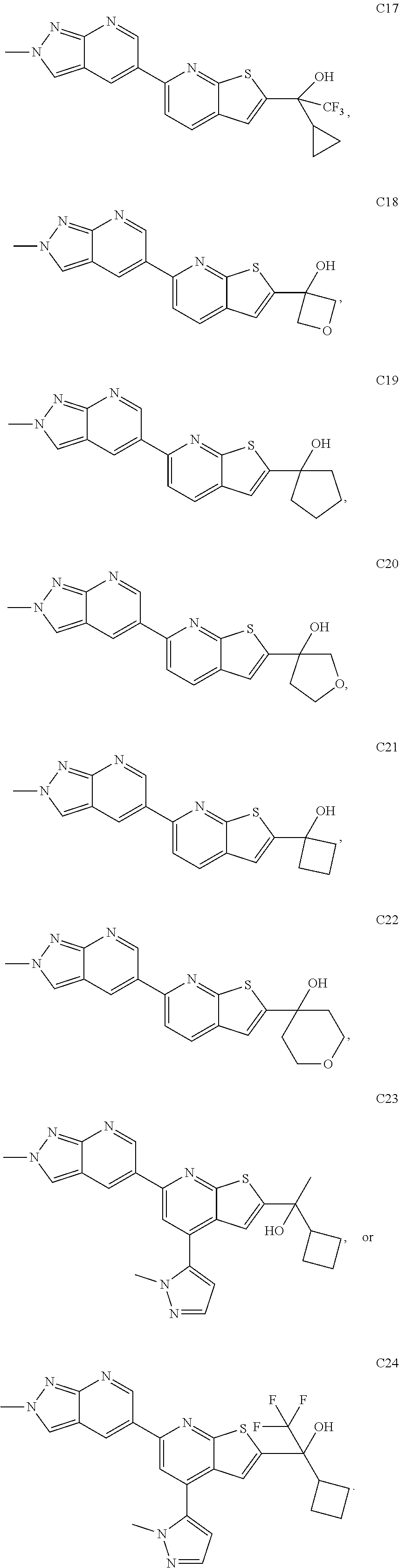

A206. The compound or salt of Embodiment A1, wherein the compound is:

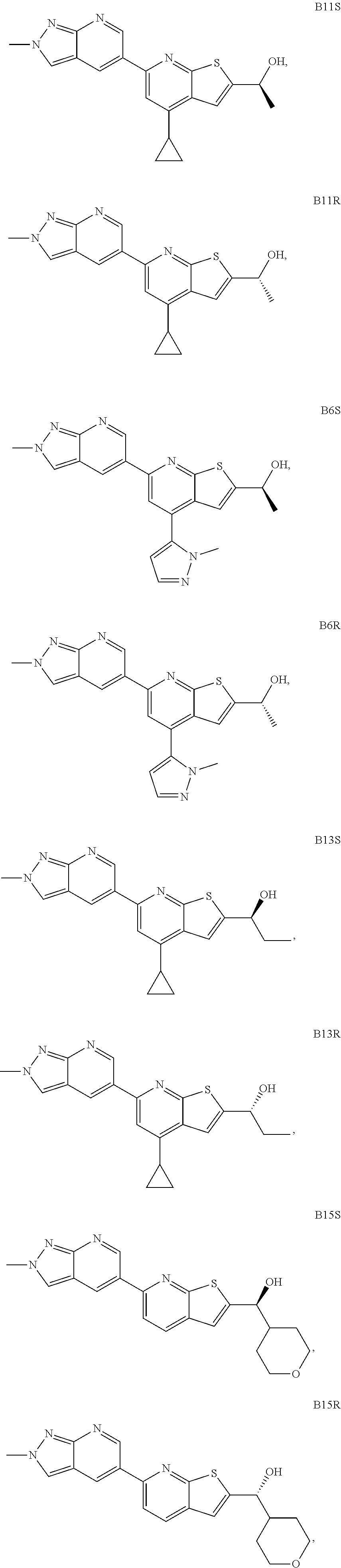

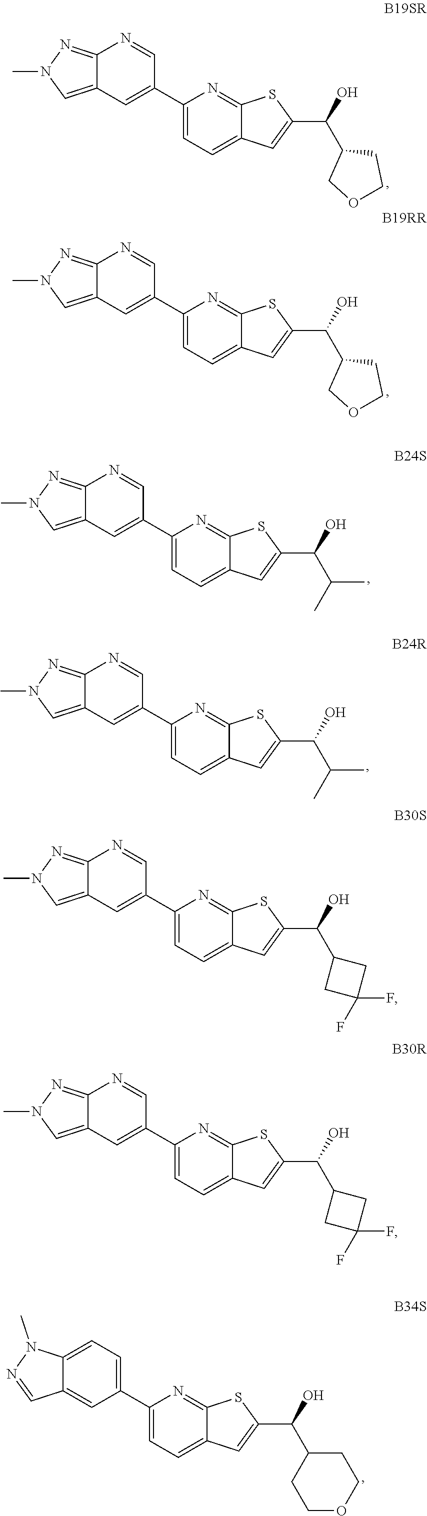

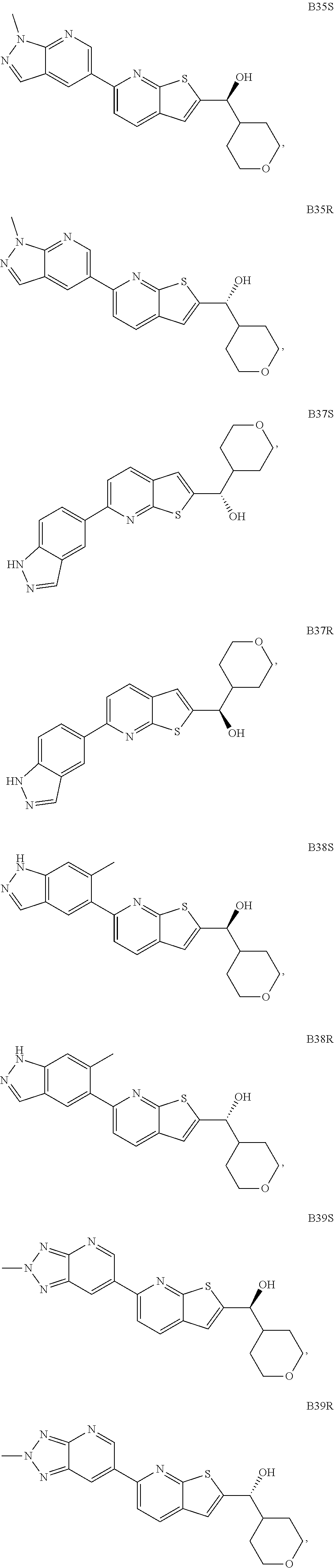

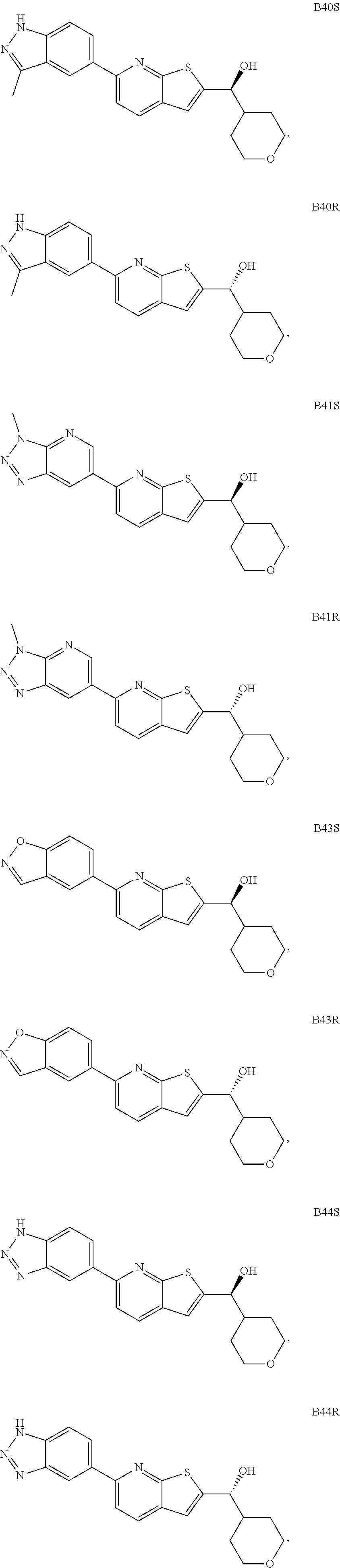

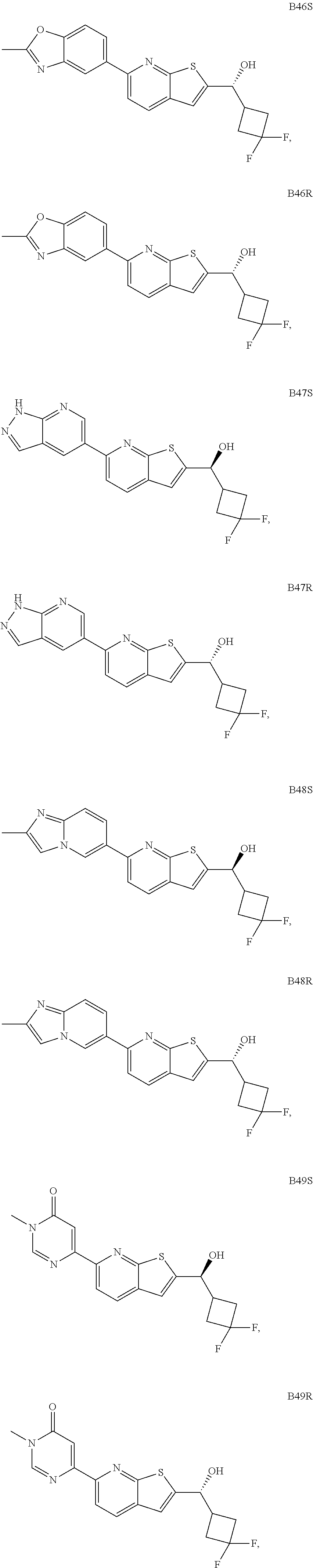

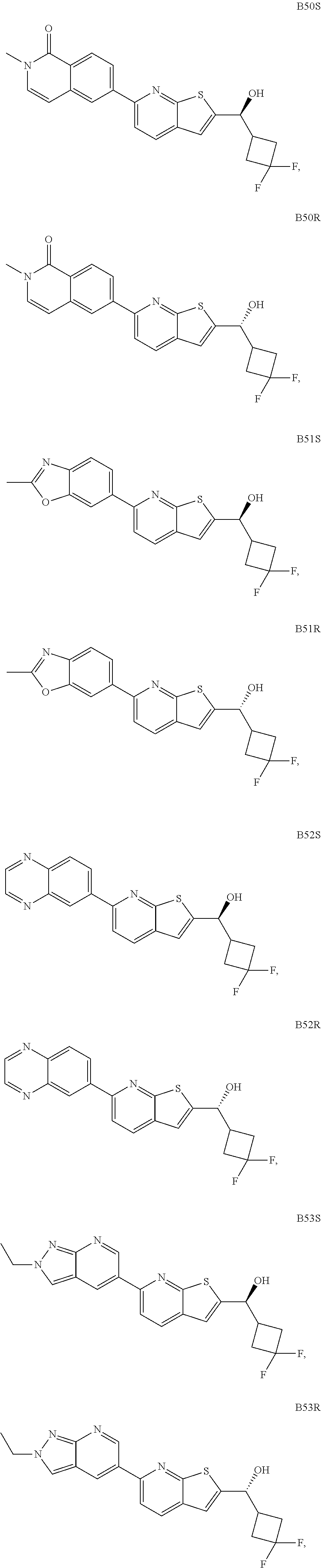

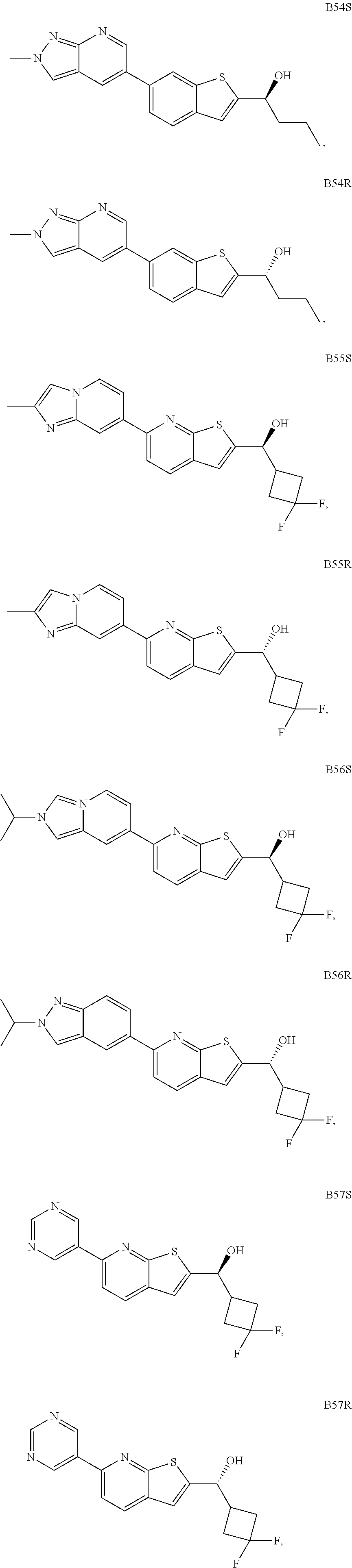

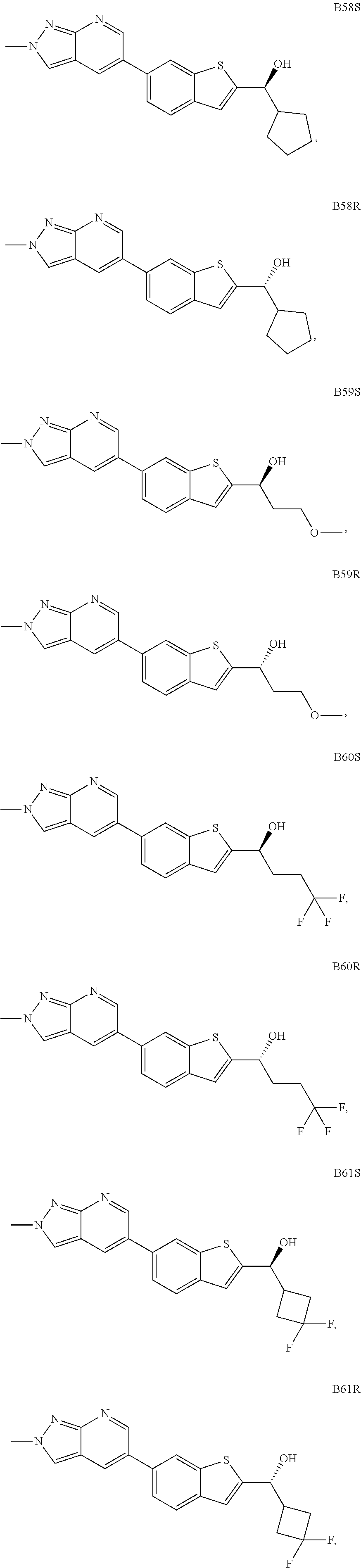

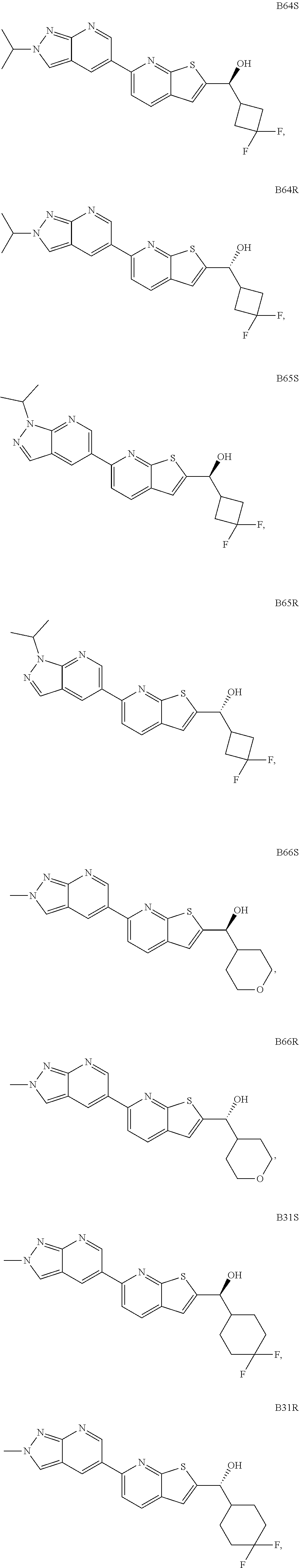

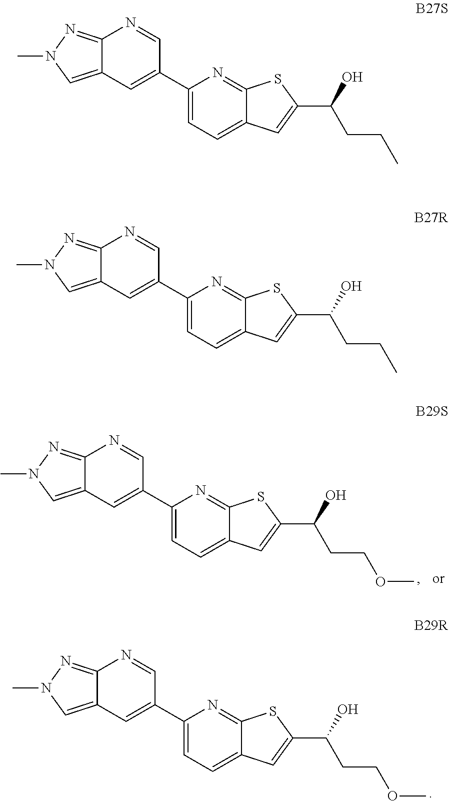

A207. The compound or salt of Embodiment A1, wherein the compound is:
5-(4-cyclobutyl-2-(methoxymethyl)thieno[2,3-b]pyridin-6-yl)-2-methyl-2H-pyrazolo[3,4-b]pyridine;
5-(2-(methoxymethyl)-4-(1-(methoxymethyl)cyclobutyl)thieno[2,3-b]pyridin-6-yl)-2-methyl-2H-pyrazolo[3,4-b]pyridine;
(R)-(4,6-bis(1-methyl-1H-pyrazol-5-yl)thieno[2,3-b]pyridin-2-yl)(cyclobutyl)methanol;
(S)-(4,6-bis(1-methyl-1H-pyrazol-5-yl)thieno[2,3-b]pyridin-2-yl)(cyclobutyl)methanol;
(R)-cyclobutyl(6-(2-methyl-2H-pyrazolo[3,4-b]pyridin-5-yl)-4-(1-methyl-1H-pyrazol-5-yl)thieno[2,3-b]pyridin-2-yl)methanol;
(S)-cyclobutyl(6-(2-methyl-2H-pyrazolo[3,4-b]pyridin-5-yl)-4-(1-methyl-1H-pyrazol-5-yl)thieno[2,3-b]pyridin-2-yl)methanol;
(R)-cyclobutyl(4-(2-methyl-2H-pyrazolo[3,4-b]pyridin-5-yl)-6-(1-methyl-1H-pyrazol-5-yl)thieno[2,3-b]pyridin-2-yl)methanol;
(S)-cyclobutyl(4-(2-methyl-2H-pyrazolo[3,4-b]pyridin-5-yl)-6-(1-methyl-1H-pyrazol-5-yl)thieno[2,3-b]pyridin-2-yl)methanol;
5-(2-((R)-cyclobutyl(methoxy)methyl)-4-(1-methyl-1H-pyrazol-5-yl)thieno[2,3-b]pyridin-6-yl)-2-methyl-2H-pyrazolo[3,4-b]pyridine;
5-(2-((S)-cyclobutyl(methoxy)methyl)-4-(1-methyl-1H-pyrazol-5-yl)thieno[2,3-b]pyridin-6-yl)-2-methyl-2H-pyrazolo[3,4-b]pyridine;
(1R)-1-cyclobutyl-1-(6-(2-methyl-2H-pyrazolo[3,4-b]pyridin-5-yl)-4-(1-methyl-1H-pyrazol-5-yl)thieno[2,3-b]pyridin-2-yl)ethanol;
(1S)-1-cyclobutyl-1-(6-(2-methyl-2H-pyrazolo[3,4-b]pyridin-5-yl)-4-(1-methyl-1H-pyrazol-5-yl)thieno[2,3-b]pyridin-2-yl)ethanol;
(1R)-1-cyclobutyl-2,2,2-trifluoro-1-(6-(2-methyl-2H-pyrazolo[3,4-b]pyridin-5-yl)-4-(1-methyl-1H-pyrazol-5-yl)thieno[2,3-b]pyridin-2-yl)ethanol;
(1S)-1-cyclobutyl-2,2,2-trifluoro-1-(6-(2-methyl-2H-pyrazolo[3,4-b]pyridin-5-yl)-4-(1-methyl-1H-pyrazol-5-yl)thieno[2,3-b]pyridin-2-yl)ethanol;
(1R)-1-(4-(2-methyl-2H-pyrazolo[3,4-b]pyridin-5-yl)-6-(1-methyl-1H-pyrazol-5-yl)thieno[2,3-b]pyridin-2-yl)ethanol;
(1S)-1-(4-(2-methyl-2H-pyrazolo[3,4-b]pyridin-5-yl)-6-(1-methyl-1H-pyrazol-5-yl)thieno[2,3-b]pyridin-2-yl)ethanol;
(1R)-1-(6-(2-methyl-2H-pyrazolo[3,4-b]pyridin-5-yl)-4-(1-methyl-1H-pyrazol-5-yl)thieno[2,3-b]pyridin-2-yl)ethanol;
(1S)-1-(6-(2-methyl-2H-pyrazolo[3,4-b]pyridin-5-yl)-4-(1-methyl-1H-pyrazol-5-yl)thieno[2,3-b]pyridin-2-yl)ethanol;
(R)-cyclobutyl(6-cyclopropyl-4-(2-methyl-2H-pyrazolo[3,4-b]pyridin-5-yl)thieno[2,3-b]pyridin-2-yl)methanol;
(S)-cyclobutyl(6-cyclopropyl-4-(2-methyl-2H-pyrazolo[3,4-b]pyridin-5-yl)thieno[2,3-b]pyridin-2-yl)methanol;
(R)-cyclobutyl(4-cyclopropyl-6-(2-methyl-2H-pyrazolo[3,4-b]pyridin-5-yl)thieno[2,3-b]pyridin-2-yl)methanol;
(S)-cyclobutyl(4-cyclopropyl-6-(2-methyl-2H-pyrazolo[3,4-b]pyridin-5-yl)thieno[2,3-b]pyridin-2-yl)methanol;
(4-(dimethylamino)-6-(2-methyl-2H-pyrazolo[3,4-b]pyridin-5-yl)thieno[2,3-b]pyridine-2,5-diyl)dimethanol;
(1R)-1-(4,6-bis(1-methyl-1H-pyrazol-5-yl)thieno[2,3-b]pyridin-2-yl)ethanol;
(1S)-1-(4,6-bis(1-methyl-1H-pyrazol-5-yl)thieno[2,3-b]pyridin-2-yl)ethanol;
(1R)-1-(4,6-bis(2-methyl-2H-pyrazolo[3,4-b]pyridin-5-yl)thieno[2,3-b]pyridin-2-yl)ethanol;
(1S)-1-(4,6-bis(2-methyl-2H-pyrazolo[3,4-b]pyridin-5-yl)thieno[2,3-b]pyridin-2-yl)ethanol;
(1R)-1-(4-cyclopropyl-6-(2-methyl-2H-pyrazolo[3,4-b]pyridin-5-yl)thieno[2,3-b]pyridin-2-yl)ethanol;
(1S)-1-(4-cyclopropyl-6-(2-methyl-2H-pyrazolo[3,4-b]pyridin-5-yl)thieno[2,3-b]pyridin-2-yl)ethanol;
(1R)-1-(6-cyclopropyl-4-(2-methyl-2H-pyrazolo[3,4-b]pyridin-5-yl)thieno[2,3-b]pyridin-2-yl)ethanol;
(1S)-1-(6-cyclopropyl-4-(2-methyl-2H-pyrazolo[3,4-b]pyridin-5-yl)thieno[2,3-b]pyridin-2-yl)ethanol;
(1R)-1-(4-cyclopropyl-6-(2-methyl-2H-pyrazolo[3,4-b]pyridin-5-yl)thieno[2,3-b]pyridin-2-yl)-1-propanol;
(1S)-1-(4-cyclopropyl-6-(2-methyl-2H-pyrazolo[3,4-b]pyridin-5-yl)thieno[2,3-b]pyridin-2-yl)-1-propanol;
(R)-(6-(2-methyl-2H-pyrazolo[3,4-b]pyridin-5-yl)thieno[2,3-b]pyridin-2-yl)(tetrahydro-2H-pyran-4-yl)methanol;
(S)-(6-(2-methyl-2H-pyrazolo[3,4-b]pyridin-5-yl)thieno[2,3-b]pyridin-2-yl)(tetrahydro-2H-pyran-4-yl)methanol;
(R)-cyclopropyl(6-(2-methyl-2H-pyrazolo[3,4-b]pyridin-5-yl)thieno[2,3-b]pyridin-2-yl)methanol;
(S)-cyclopropyl(6-(2-methyl-2H-pyrazolo[3,4-b]pyridin-5-yl)thieno[2,3-b]pyridin-2-yl)methanol;
(R)-cyclobutyl(6-(2-methyl-2H-pyrazolo[3,4-b]pyridin-5-yl)thieno[2,3-b]pyridin-2-yl)methanol;
(S)-cyclobutyl(6-(2-methyl-2H-pyrazolo[3,4-b]pyridin-5-yl)thieno[2,3-b]pyridin-2-yl)methanol;
(3R)-3-(6-(2-methyl-2H-pyrazolo[3,4-b]pyridin-5-yl)thieno[2,3-b]pyridin-2-yl)tetrahydro-3-furanol;
(3S)-3-(6-(2-methyl-2H-pyrazolo[3,4-b]pyridin-5-yl)thieno[2,3-b]pyridin-2-yl)tetrahydro-3-furanol;
1-(6-(2-methyl-2H-pyrazolo[3,4-b]pyridin-5-yl)thieno[2,3-b]pyridin-2-yl)cyclobutanol;
4-(6-(2-methyl-2H-pyrazolo[3,4-b]pyridin-5-yl)thieno[2,3-b]pyridin-2-yl)tetrahydro-2H-pyran-4-ol;
(R)-(6-(2-methyl-2H-pyrazolo[3,4-b]pyridin-5-yl)thieno[2,3-b]pyridin-2-yl)((3R)-tetrahydro-3-furanyl)methanol;
(R)-(6-(2-methyl-2H-pyrazolo[3,4-b]pyridin-5-yl)thieno[2,3-b]pyridin-2-yl)((3S)-tetrahydro-3-furanyl)methanol;
(S)-(6-(2-methyl-2H-pyrazolo[3,4-b]pyridin-5-yl)thieno[2,3-b]pyridin-2-yl)((3R)-tetrahydro-3-furanyl)methanol;
(S)-(6-(2-methyl-2H-pyrazolo[3,4-b]pyridin-5-yl)thieno[2,3-b]pyridin-2-yl)((3S)-tetrahydro-3-furanyl)methanol;
3-(6-(2-methyl-2H-pyrazolo[3,4-b]pyridin-5-yl)thieno[2,3-b]pyridin-2-yl)-3-oxetanol;
1-(6-(2-methyl-2H-pyrazolo[3,4-b]pyridin-5-yl)thieno[2,3-b]pyridin-2-yl)cyclopentanol;
(1R)-1-(6-(2-methyl-2H-pyrazolo[3,4-b]pyridin-5-yl)thieno[2,3-b]pyridin-2-yl)-1-propanol;
(1S)-1-(6-(2-methyl-2H-pyrazolo[3,4-b]pyridin-5-yl)thieno[2,3-b]pyridin-2-yl)-1-propanol;
(6-(2-methyl-2H-pyrazolo[3,4-b]pyridin-5-yl)thieno[2,3-b]pyridin-2-yl)methanol;
(1R)-2,2,2-trifluoro-1-(6-(2-methyl-2H-pyrazolo[3,4-b]pyridin-5-yl)thieno[2,3-b]pyridin-2-yl)ethanol;
(1S)-2,2,2-trifluoro-1-(6-(2-methyl-2H-pyrazolo[3,4-b]pyridin-5-yl)thieno[2,3-b]pyridin-2-yl)ethanol;
(1R)-2-methyl-1-(6-(2-methyl-2H-pyrazolo[3,4-b]pyridin-5-yl)thieno[2,3-b]pyridin-2-yl)-1-propanol;
(1S)-2-methyl-1-(6-(2-methyl-2H-pyrazolo[3,4-b]pyridin-5-yl)thieno[2,3-b]pyridin-2-yl)-1-propanol;
(1R)-1-(6-(2-methyl-2H-pyrazolo[3,4-b]pyridin-5-yl)thieno[2,3-b]pyridin-2-yl)ethanol;
(1S)-1-(6-(2-methyl-2H-pyrazolo[3,4-b]pyridin-5-yl)thieno[2,3-b]pyridin-2-yl)ethanol;
(1R)-1-(6-(2-methyl-2H-pyrazolo[3,4-b]pyridin-5-yl)thieno[2,3-b]pyridin-2-yl)-1-butanol;
(1S)-1-(6-(2-methyl-2H-pyrazolo[3,4-b]pyridin-5-yl)thieno[2,3-b]pyridin-2-yl)-1-butanol;
(R)-(6-(2-methyl-2H-pyrazolo[3,4-b]pyridin-5-yl)thieno[2,3-b]pyridin-2-yl)(3-oxetanyl)methanol;
(S)-(6-(2-methyl-2H-pyrazolo[3,4-b]pyridin-5-yl)thieno[2,3-b]pyridin-2-yl)(3-oxetanyl)methanol;
(1R)-3-methoxy-1-(6-(2-methyl-2H-pyrazolo[3,4-b]pyridin-5-yl)thieno[2,3-b]pyridin-2-yl)-1-propanol;
(1S)-3-methoxy-1-(6-(2-methyl-2H-pyrazolo[3,4-b]pyridin-5-yl)thieno[2,3-b]pyridin-2-yl)-1-propanol;
4,4-difluoro-1-(6-(2-methyl-2H-pyrazolo[3,4-b]pyridin-5-yl)thieno[2,3-b]pyridin-2-yl)cyclohexanol;
(2R)-1,1,1-trifluoro-3-methoxy-2-(6-(2-methyl-2H-pyrazolo[3,4-b]pyridin-5-yl)thieno[2,3-b]pyridin-2-yl)-2-propanol;
(2S)-1,1,1-trifluoro-3-methoxy-2-(6-(2-methyl-2H-pyrazolo[3,4-b]pyridin-5-yl)thieno[2,3-b]pyridin-2-yl)-2-propanol;
(1R)-1-cyclopropyl-2,2,2-trifluoro-1-(6-(2-methyl-2H-pyrazolo[3,4-b]pyridin-5-yl)thieno[2,3-b]pyridin-2-yl)ethanol;
(1S)-1-cyclopropyl-2,2,2-trifluoro-1-(6-(2-methyl-2H-pyrazolo[3,4-b]pyridin-5-yl)thieno[2,3-b]pyridin-2-yl)ethanol;
(R)-(3,3-difluorocyclobutyl)(6-(2-methyl-2H-pyrazolo[3,4-b]pyridin-5-yl)thieno[2,3-b]pyridin-2-yl)methanol;
(S)-(3,3-difluorocyclobutyl)(6-(2-methyl-2H-pyrazolo[3,4-b]pyridin-5-yl)thieno[2,3-b]pyridin-2-yl)methanol;
(R)-(4,4-difluorocyclohexyl)(6-(2-methyl-2H-pyrazolo[3,4-b]pyridin-5-yl)thieno[2,3-b]pyridin-2-yl)methanol;
(S)-(4,4-difluorocyclohexyl)(6-(2-methyl-2H-pyrazolo[3,4-b]pyridin-5-yl)thieno[2,3-b]pyridin-2-yl)methanol;
1-methyl-3-(6-(2-methyl-2H-pyrazolo[3,4-b]pyridin-5-yl)thieno[2,3-b]pyridin-2-yl)-3-azetidinol;
(3R)-1-methyl-3-(6-(2-methyl-2H-pyrazolo[3,4-b]pyridin-5-yl)thieno[2,3-b]pyridin-2-yl)-3-piperidinol;
(3S)-1-methyl-3-(6-(2-methyl-2H-pyrazolo[3,4-b]pyridin-5-yl)thieno[2,3-b]pyridin-2-yl)-3-piperidinol;
(2R)-1,1,1-trifluoro-2-(6-(2-methyl-2H-pyrazolo[3,4-b]pyridin-5-yl)thieno[2,3-b]pyridin-2-yl)-2-butanol;
(2S)-1,1,1-trifluoro-2-(6-(2-methyl-2H-pyrazolo[3,4-b]pyridin-5-yl)thieno[2,3-b]pyridin-2-yl)-2-butanol;
(1R)-2-methoxy-1-(6-(2-methyl-2H-pyrazolo[3,4-b]pyridin-5-yl)thieno[2,3-b]pyridin-2-yl)ethanol;
(1S)-2-methoxy-1-(6-(2-methyl-2H-pyrazolo[3,4-b]pyridin-5-yl)thieno[2,3-b]pyridin-2-yl)ethanol;
4-(6-(2-methyl-2H-pyrazolo[3,4-b]pyridin-5-yl)thieno[2,3-b]pyridin-2-yl)tetrahydro-2H-thiopyran-4-ol 1,1-dioxide;
2-(6-(2-methyl-2H-pyrazolo[3,4-b]pyridin-5-yl)thieno[2,3-b]pyridin-2-yl)-2-propanol;
(2R)-2-(6-(2-methyl-2H-pyrazolo[3,4-b]pyridin-5-yl)thieno[2,3-b]pyridin-2-yl)-2-butanol;
(2S)-2-(6-(2-methyl-2H-pyrazolo[3,4-b]pyridin-5-yl)thieno[2,3-b]pyridin-2-yl)-2-butanol;
(1R)-1-cyclopropyl-1-(6-(2-methyl-2H-pyrazolo[3,4-b]pyridin-5-yl)thieno[2,3-b]pyridin-2-yl)ethanol;
(1S)-1-cyclopropyl-1-(6-(2-methyl-2H-pyrazolo[3,4-b]pyridin-5-yl)thieno[2,3-b]pyridin-2-yl)ethanol;
1-methyl-4-(6-(2-methyl-2H-pyrazolo[3,4-b]pyridin-5-yl)thieno[2,3-b]pyridin-2-yl)-4-piperidinol;
(2R)-1-methoxy-2-(6-(2-methyl-2H-pyrazolo[3,4-b]pyridin-5-yl)thieno[2,3-b]pyridin-2-yl)-2-propanol;
(2S)-1-methoxy-2-(6-(2-methyl-2H-pyrazolo[3,4-b]pyridin-5-yl)thieno[2,3-b]pyridin-2-yl)-2-propanol;
3,3-difluoro-1-(6-(2-methyl-2H-pyrazolo[3,4-b]pyridin-5-yl)thieno[2,3-b]pyridin-2-yl)cyclobutanol;
(3R)-1-methyl-3-(6-(2-methyl-2H-pyrazolo[3,4-b]pyridin-5-yl)thieno[2,3-b]pyridin-2-yl)-3-pyrrolidinol;
(3S)-1-methyl-3-(6-(2-methyl-2H-pyrazolo[3,4-b]pyridin-5-yl)thieno[2,3-b]pyridin-2-yl)-3-pyrrolidinol;
(R)-(1-methyl-4-piperidinyl)(6-(2-methyl-2H-pyrazolo[3,4-b]pyridin-5-yl)thieno[2,3-b]pyridin-2-yl)methanol;
(S)-(1-methyl-4-piperidinyl)(6-(2-methyl-2H-pyrazolo[3,4-b]pyridin-5-yl)thieno[2,3-b]pyridin-2-yl)methanol;
(S)-(6-(1-methyl-1H-indazol-5-yl)thieno[2,3-b]pyridin-2-yl)(tetrahydro-2H-pyran-4-yl)methanol;
(R)-(6-(1-methyl-1H-indazol-5-yl)thieno[2,3-b]pyridin-2-yl)(tetrahydro-2H-pyran-4-yl)methanol;
(S)-(6-(1-methyl-1H-pyrazolo[3,4-b]pyridin-5-yl)thieno[2,3-b]pyridin-2-yl)(tetrahydro-2H-pyran-4-yl)methanol;
(R)-(6-(1-methyl-1H-pyrazolo[3,4-b]pyridin-5-yl)thieno[2,3-b]pyridin-2-yl)(tetrahydro-2H-pyran-4-yl)methanol;
(2R)-1,1,1-trifluoro-4-methoxy-2-(6-(2-methyl-2H-pyrazolo[3,4-b]pyridin-5-yl)thieno[2,3-b]pyridin-2-yl)-2-butanol;
(2S)-1,1,1-trifluoro-4-methoxy-2-(6-(2-methyl-2H-pyrazolo[3,4-b]pyridin-5-yl)thieno[2,3-b]pyridin-2-yl)-2-butanol;
(S)-(6-(1H-indazol-5-yl)thieno[2,3-b]pyridin-2-yl)(tetrahydro-2H-pyran-4-yl)methanol;
(R)-(6-(1H-indazol-5-yl)thieno[2,3-b]pyridin-2-yl)(tetrahydro-2H-pyran-4-yl)methanol;
(S)-(6-(6-methyl-1H-indazol-5-yl)thieno[2,3-b]pyridin-2-yl)(tetrahydro-2H-pyran-4-yl)methanol;
(R)-(6-(6-methyl-1H-indazol-5-yl)thieno[2,3-b]pyridin-2-yl)(tetrahydro-2H-pyran-4-yl)methanol;
(2R)-1,1,1-trifluoro-2-(6-(2-methyl-2H-pyrazolo[3,4-b]pyridin-5-yl)thieno[2,3-b]pyridin-2-yl)-2-propanol;
(2S)-1,1,1-trifluoro-2-(6-(2-methyl-2H-pyrazolo[3,4-b]pyridin-5-yl)thieno[2,3-b]pyridin-2-yl)-2-propanol;
(S)-(6-(2-methyl-2H-[1,2,3]triazolo[4,5-b]pyridin-6-yl)thieno[2,3-b]pyridin-2-yl)(tetrahydro-2H-pyran-4-yl)methanol;
(R)-(6-(2-methyl-2H-[1,2,3]triazolo[4,5-b]pyridin-6-yl)thieno[2,3-b]pyridin-2-yl)(tetrahydro-2H-pyran-4-yl)methanol;
(S)-(6-(3-methyl-1H-indazol-5-yl)thieno[2,3-b]pyridin-2-yl)(tetrahydro-2H-pyran-4-yl)methanol;
(R)-(6-(3-methyl-1H-indazol-5-yl)thieno[2,3-b]pyridin-2-yl)(tetrahydro-2H-pyran-4-yl)methanol;
(2R)-4-methoxy-2-(6-(2-methyl-2H-pyrazolo[3,4-b]pyridin-5-yl)thieno[2,3-b]pyridin-2-yl)-2-butanol;
(2S)-4-methoxy-2-(6-(2-methyl-2H-pyrazolo[3,4-b]pyridin-5-yl)thieno[2,3-b]pyridin-2-yl)-2-butanol;
(R)-(6-(3-methyl-3H-[1,2,3]triazolo[4,5-b]pyridin-6-yl)thieno[2,3-b]pyridin-2-yl)(tetrahydro-2H-pyran-4-yl)methanol;
(S)-(6-(3-methyl-3H-[1,2,3]triazolo[4,5-b]pyridin-6-yl)thieno[2,3-b]pyridin-2-yl)(tetrahydro-2H-pyran-4-yl)methanol;
(R)-(1-methyl-3-azetidinyl)(6-(2-methyl-2H-pyrazolo[3,4-b]pyridin-5-yl)thieno[2,3-b]pyridin-2-yl)methanol;
(S)-(1-methyl-3-azetidinyl)(6-(2-methyl-2H-pyrazolo[3,4-b]pyridin-5-yl)thieno[2,3-b]pyridin-2-yl)methanol;
(S)-(6-(1,2-benzoxazol-5-yl)thieno[2,3-b]pyridin-2-yl)(tetrahydro-2H-pyran-4-yl)methanol;
(R)-(6-(1,2-benzoxazol-5-yl)thieno[2,3-b]pyridin-2-yl)(tetrahydro-2H-pyran-4-yl)methanol;
(S)-tetrahydro-2H-pyran-4-yl(6-(3H-[1,2,3]triazolo[4,5-b]pyridin-6-yl)thieno[2,3-b]pyridin-2-yl)methanol;
(R)-tetrahydro-2H-pyran-4-yl(6-(3H-[1,2,3]triazolo[4,5-b]pyridin-6-yl)thieno[2,3-b]pyridin-2-yl)methanol;
(R)-((2R)-1-methyl-2-azetidinyl)(6-(2-methyl-2H-pyrazolo[3,4-b]pyridin-5-yl)thieno[2,3-b]pyridin-2-yl)methanol;
(R)-((2S)-1-methyl-2-azetidinyl)(6-(2-methyl-2H-pyrazolo[3,4-b]pyridin-5-yl)thieno[2,3-b]pyridin-2-yl)methanol;
(S)-((2R)-1-methyl-2-azetidinyl)(6-(2-methyl-2H-pyrazolo[3,4-b]pyridin-5-yl)thieno[2,3-b]pyridin-2-yl)methanol;
(S)-((2S)-1-methyl-2-azetidinyl)(6-(2-methyl-2H-pyrazolo[3,4-b]pyridin-5-yl)thieno[2,3-b]pyridin-2-yl)methanol;
(S)-(3,3-difluorocyclobutyl)(6-(2-methyl-1,3-benzoxazol-5-yl)thieno[2,3-b]pyridin-2-yl)methanol;
(R)-(3,3-difluorocyclobutyl)(6-(2-methyl-1,3-benzoxazol-5-yl)thieno[2,3-b]pyridin-2-yl)methanol;
(S)-(3,3-difluorocyclobutyl)(6-(1H-pyrazolo[3,4-b]pyridin-5-yl)thieno[2,3-b]pyridin-2-yl)methanol;
(R)-(3,3-difluorocyclobutyl)(6-(1H-pyrazolo[3,4-b]pyridin-5-yl)thieno[2,3-b]pyridin-2-yl)methanol;
(S)-(3,3-difluorocyclobutyl)(6-(2-methylimidazo[1,2-a]pyridin-6-yl)thieno[2,3-b]pyridin-2-yl)methanol;
(R)-(3,3-difluorocyclobutyl)(6-(2-methylimidazo[1,2-a]pyridin-6-yl)thieno[2,3-b]pyridin-2-yl)methanol;
6-(2-((R)-(3,3-difluorocyclobutyl)(hydroxy)methyl)thieno[2,3-b]pyridin-6-yl)-3-methyl-4(3H)-pyrimidinone;
6-(2-((S)-(3,3-difluorocyclobutyl)(hydroxy)methyl)thieno[2,3-b]pyridin-6-yl)-3-methyl-4(3H)-pyrimidinone;
6-(2-((S)-(3,3-difluorocyclobutyl)(hydroxy)methyl)thieno[2,3-b]pyridin-6-yl)-2-methyl-1(2H)-isoquinolinone;
6-(2-((R)-(3,3-difluorocyclobutyl)(hydroxy)methyl)thieno[2,3-b]pyridin-6-yl)-2-methyl-1(2H)-isoquinolinone;
(S)-(3,3-difluorocyclobutyl)(6-(2-methyl-1,3-benzoxazol-6-yl)thieno[2,3-b]pyridin-2-yl)methanol;
(R)-(3,3-difluorocyclobutyl)(6-(2-methyl-1,3-benzoxazol-6-yl)thieno[2,3-b]pyridin-2-yl)methanol;
(S)-(3,3-difluorocyclobutyl)(6-(6-quinoxalinyl)thieno[2,3-b]pyridin-2-yl)methanol;
(R)-(3,3-difluorocyclobutyl)(6-(6-quinoxalinyl)thieno[2,3-b]pyridin-2-yl)methanol;
(S)-(3,3-difluorocyclobutyl)(6-(2-ethyl-2H-pyrazolo[3,4-b]pyridin-5-yl)thieno[2,3-b]pyridin-2-yl)methanol;
(R)-(3,3-difluorocyclobutyl)(6-(2-ethyl-2H-pyrazolo[3,4-b]pyridin-5-yl)thieno[2,3-b]pyridin-2-yl)methanol;
(1S)-1-(6-(2-methyl-2H-pyrazolo[3,4-b]pyridin-5-yl)-1-benzothiophen-2-yl)-1-butanol;
(1R)-1-(6-(2-methyl-2H-pyrazolo[3,4-b]pyridin-5-yl)-1-benzothiophen-2-yl)-1-butanol;
(S)-(3,3-difluorocyclobutyl)(6-(2-methylimidazo[1,2-a]pyridin-7-yl)thieno[2,3-b]pyridin-2-yl)methanol;
(R)-(3,3-difluorocyclobutyl)(6-(2-methylimidazo[1,2-a]pyridin-7-yl)thieno[2,3-b]pyridin-2-yl)methanol;
(S)-(3,3-difluorocyclobutyl)(6-(2-(2-propanyl)-2H-indazol-5-yl)thieno[2,3-b]pyridin-2-yl)methanol;
(R)-(3,3-difluorocyclobutyl)(6-(2-(2-propanyl)-2H-indazol-5-yl)thieno[2,3-b]pyridin-2-yl)methanol;
(S)-(3,3-difluorocyclobutyl)(6-(5-pyrimidinyl)thieno[2,3-b]pyridin-2-yl)methanol;
(R)-(3,3-difluorocyclobutyl)(6-(5-pyrimidinyl)thieno[2,3-b]pyridin-2-yl)methanol;
(S)-cyclopentyl(6-(2-methyl-2H-pyrazolo[3,4-b]pyridin-5-yl)-1-benzothiophen-2-yl)methanol;
(R)-cyclopentyl(6-(2-methyl-2H-pyrazolo[3,4-b]pyridin-5-yl)-1-benzothiophen-2-yl)methanol;
(1S)-3-methoxy-1-(6-(2-methyl-2H-pyrazolo[3,4-b]pyridin-5-yl)-1-benzothiophen-2-yl)-1-propanol;
(1R)-3-methoxy-1-(6-(2-methyl-2H-pyrazolo[3,4-b]pyridin-5-yl)-1-benzothiophen-2-yl)-1-propanol;
(1S)-4,4,4-trifluoro-1-(6-(2-methyl-2H-pyrazolo[3,4-b]pyridin-5-yl)-1-benzothiophen-2-yl)-1-butanol;
(1R)-4,4,4-trifluoro-1-(6-(2-methyl-2H-pyrazolo[3,4-b]pyridin-5-yl)-1-benzothiophen-2-yl)-1-butanol;
(S)-(3,3-difluorocyclobutyl)(6-(2-methyl-2H-pyrazolo[3,4-b]pyridin-5-yl)-1-benzothiophen-2-yl)methanol;
(R)-(3,3-difluorocyclobutyl)(6-(2-methyl-2H-pyrazolo[3,4-b]pyridin-5-yl)-1-benzothiophen-2-yl)methanol;
(R)-(6-(2-methyl-2H-pyrazolo[3,4-b]pyridin-5-yl)thieno[2,3-b]pyridin-2-yl)((3R)-1-methyl-3-pyrrolidinyl)methanol;
(R)-(6-(2-methyl-2H-pyrazolo[3,4-b]pyridin-5-yl)thieno[2,3-b]pyridin-2-yl)((3S)-1-methyl-3-pyrrolidinyl)methanol;
(S)-(6-(2-methyl-2H-pyrazolo[3,4-b]pyridin-5-yl)thieno[2,3-b]pyridin-2-yl)((3R)-1-methyl-3-pyrrolidinyl)methanol;
(S)-(6-(2-methyl-2H-pyrazolo[3,4-b]pyridin-5-yl)thieno[2,3-b]pyridin-2-yl)((3S)-1-methyl-3-pyrrolidinyl)methanol;
(R)-((3R)-1-methyl-3-piperidinyl)(6-(2-methyl-2H-pyrazolo[3,4-b]pyridin-5-yl)thieno[2,3-b]pyridin-2-yl)methanol;
(R)-((3S)-1-methyl-3-piperidinyl)(6-(2-methyl-2H-pyrazolo[3,4-b]pyridin-5-yl)thieno[2,3-b]pyridin-2-yl)methanol;
(S)-((3R)-1-methyl-3-piperidinyl)(6-(2-methyl-2H-pyrazolo[3,4-b]pyridin-5-yl)thieno[2,3-b]pyridin-2-yl)methanol;
(S)-((3S)-1-methyl-3-piperidinyl)(6-(2-methyl-2H-pyrazolo[3,4-b]pyridin-5-yl)thieno[2,3-b]pyridin-2-yl)methanol;
(S)-(3,3-difluorocyclobutyl)(6-(2-(2-propanyl)-2H-pyrazolo[3,4-b]pyridin-5-yl)thieno[2,3-b]pyridin-2-yl)methanol;
(R)-(3,3-difluorocyclobutyl)(6-(2-(2-propanyl)-2H-pyrazolo[3,4-b]pyridin-5-yl)thieno[2,3-b]pyridin-2-yl)methanol;
(S)-(3,3-difluorocyclobutyl)(6-(1-(2-propanyl)-1H-pyrazolo[3,4-b]pyridin-5-yl)thieno[2,3-b]pyridin-2-yl)methanol;
(R)-(3,3-difluorocyclobutyl)(6-(1-(2-propanyl)-1H-pyrazolo[3,4-b]pyridin-5-yl)thieno[2,3-b]pyridin-2-yl)methanol;
(R)-(6-(2-methyl-2H-pyrazolo[3,4-b]pyridin-5-yl)thieno[2,3-b]pyridin-2-yl)(tetrahydro-2H-pyran-4-yl)methan-d-ol;
(S)-(6-(2-methyl-2H-pyrazolo[3,4-b]pyridin-5-yl)thieno[2,3-b]pyridin-2-yl)(tetrahydro-2H-pyran-4-yl)methan-d-ol;
(R)-(3-amino-6-(5-pyrimidinyl)thieno[2,3-b]pyridin-2-yl)(3,3-difluorocyclobutyl)methanol; and
(S)-(3-amino-6-(5-pyrimidinyl)thieno[2,3-b]pyridin-2-yl)(3,3-difluorocyclobutyl)methanol.
A208. The compound or salt of Embodiment A1, wherein the compound is:
5-(4-cyclobutyl-2-(methoxymethyl)thieno[2,3-b]pyridin-6-yl)-2-methyl-2H-pyrazolo[3,4-b]pyridine;
5-(2-(methoxymethyl)-4-(1-(methoxymethyl)cyclobutyl)thieno[2,3-b]pyridin-6-yl)-2-methyl-2H-pyrazolo[3,4-b]pyridine;
(4,6-bis(1-methyl-1H-pyrazol-5-yl)thieno[2,3-b]pyridin-2-yl)(cyclobutyl)methanol;
cyclobutyl(6-(2-methyl-2H-pyrazolo[3,4-b]pyridin-5-yl)-4-(1-methyl-1H-pyrazol-5-yl)thieno[2,3-b]pyridin-2-yl)methanol;
cyclobutyl(4-(2-methyl-2H-pyrazolo[3,4-b]pyridin-5-yl)-6-(1-methyl-1H-pyrazol-5-yl)thieno[2,3-b]pyridin-2-yl)methanol;
5-(2-(cyclobutyl(methoxy)methyl)-4-(1-methyl-1H-pyrazol-5-yl)thieno[2,3-b]pyridin-6-yl)-2-methyl-2H-pyrazolo [3,4-b]pyridine;
1-cyclobutyl-1-(6-(2-methyl-2H-pyrazolo[3,4-b]pyridin-5-yl)-4-(1-methyl-1H-pyrazol-5-yl)thieno[2,3-b]pyridin-2-yl)ethanol;
1-cyclobutyl-2,2,2-trifluoro-1-(6-(2-methyl-2H-pyrazolo[3,4-b]pyridin-5-yl)-4-(1-methyl-1H-pyrazol-5-yl)thieno[2,3-b]pyridin-2-yl)ethanol;
1-(4-(2-methyl-2H-pyrazolo[3,4-b]pyridin-5-yl)-6-(1-methyl-1H-pyrazol-5-yl)thieno[2,3-b]pyridin-2-yl)ethanol;
1-(6-(2-methyl-2H-pyrazolo[3,4-b]pyridin-5-yl)-4-(1-methyl-1H-pyrazol-5-yl)thieno[2,3-b]pyridin-2-yl)ethanol;
cyclobutyl(6-cyclopropyl-4-(2-methyl-2H-pyrazolo[3,4-b]pyridin-5-yl)thieno[2,3-b]pyridin-2-yl)methanol;
cyclobutyl(4-cyclopropyl-6-(2-methyl-2H-pyrazolo[3,4-b]pyridin-5-yl)thieno[2,3-b]pyridin-2-yl)methanol;
(4-(dimethylamino)-6-(2-methyl-2H-pyrazolo[3,4-b]pyridin-5-yl)thieno[2,3-b]pyridine-2,5-diyl)dimethanol;
1-(4,6-bis(1-methyl-1H-pyrazol-5-yl)thieno[2,3-b]pyridin-2-yl)ethanol;
1-(4,6-bis(2-methyl-2H-pyrazolo[3,4-b]pyridin-5-yl)thieno[2,3-b]pyridin-2-yl)ethanol;
1-(4-cyclopropyl-6-(2-methyl-2H-pyrazolo[3,4-b]pyridin-5-yl)thieno[2,3-b]pyridin-2-yl)ethanol;
1-(6-cyclopropyl-4-(2-methyl-2H-pyrazolo[3,4-b]pyridin-5-yl)thieno[2,3-b]pyridin-2-yl)ethanol;
1-(4-cyclopropyl-6-(2-methyl-2H-pyrazolo[3,4-b]pyridin-5-yl)thieno[2,3-b]pyridin-2-yl)-1-propanol;
(6-(2-methyl-2H-pyrazolo[3,4-b]pyridin-5-yl)thieno[2,3-b]pyridin-2-yl)(tetrahydro-2H-pyran-4-yl)methanol;
cyclopropyl(6-(2-methyl-2H-pyrazolo[3,4-b]pyridin-5-yl)thieno[2,3-b]pyridin-2-yl)methanol;
cyclobutyl(6-(2-methyl-2H-pyrazolo[3,4-b]pyridin-5-yl)thieno[2,3-b]pyridin-2-yl)methanol;
3-(6-(2-methyl-2H-pyrazolo[3,4-b]pyridin-5-yl)thieno[2,3-b]pyridin-2-yl)tetrahydro-3-furanol;
1-(6-(2-methyl-2H-pyrazolo[3,4-b]pyridin-5-yl)thieno[2,3-b]pyridin-2-yl)cyclobutanol;
4-(6-(2-methyl-2H-pyrazolo[3,4-b]pyridin-5-yl)thieno[2,3-b]pyridin-2-yl)tetrahydro-2H-pyran-4-ol;
(6-(2-methyl-2H-pyrazolo[3,4-b]pyridin-5-yl)thieno[2,3-b]pyridin-2-yl)(tetrahydro-3-furanyl)methanol;
3-(6-(2-methyl-2H-pyrazolo[3,4-b]pyridin-5-yl)thieno[2,3-b]pyridin-2-yl)-3-oxetanol;
1-(6-(2-methyl-2H-pyrazolo[3,4-b]pyridin-5-yl)thieno[2,3-b]pyridin-2-yl)cyclopentanol;
1-(6-(2-methyl-2H-pyrazolo[3,4-b]pyridin-5-yl)thieno[2,3-b]pyridin-2-yl)-1-propanol;
(6-(2-methyl-2H-pyrazolo[3,4-b]pyridin-5-yl)thieno[2,3-b]pyridin-2-yl)methanol;
2,2,2-trifluoro-1-(6-(2-methyl-2H-pyrazolo[3,4-b]pyridin-5-yl)thieno[2,3-b]pyridin-2-yl)ethanol;
2-methyl-1-(6-(2-methyl-2H-pyrazolo[3,4-b]pyridin-5-yl)thieno[2,3-b]pyridin-2-yl)-1-propanol;
1-(6-(2-methyl-2H-pyrazolo[3,4-b]pyridin-5-yl)thieno[2,3-b]pyridin-2-yl)ethanol;
1-(6-(2-methyl-2H-pyrazolo[3,4-b]pyridin-5-yl)thieno[2,3-b]pyridin-2-yl)-1-butanol;
(6-(2-methyl-2H-pyrazolo[3,4-b]pyridin-5-yl)thieno[2,3-b]pyridin-2-yl)(3-oxetanyl)methanol;
3-methoxy-1-(6-(2-methyl-2H-pyrazolo[3,4-b]pyridin-5-yl)thieno[2,3-b]pyridin-2-yl)-1-propanol;
4,4-difluoro-1-(6-(2-methyl-2H-pyrazolo[3,4-b]pyridin-5-yl)thieno[2,3-b]pyridin-2-yl)cyclohexanol;
1,1,1-trifluoro-3-methoxy-2-(6-(2-methyl-2H-pyrazolo[3,4-b]pyridin-5-yl)thieno[2,3-b]pyridin-2-yl)-2-propanol;
1-cyclopropyl-2,2,2-trifluoro-1-(6-(2-methyl-2H-pyrazolo[3,4-b]pyridin-5-yl)thieno[2,3-b]pyridin-2-yl)ethanol;
(3,3-difluorocyclobutyl)(6-(2-methyl-2H-pyrazolo[3,4-b]pyridin-5-yl)thieno[2,3-b]pyridin-2-yl)methanol;
(4,4-difluorocyclohexyl)(6-(2-methyl-2H-pyrazolo[3,4-b]pyridin-5-yl)thieno[2,3-b]pyridin-2-yl)methanol;
1-methyl-3-(6-(2-methyl-2H-pyrazolo[3,4-b]pyridin-5-yl)thieno[2,3-b]pyridin-2-yl)-3-azetidinol;
1-methyl-3-(6-(2-methyl-2H-pyrazolo[3,4-b]pyridin-5-yl)thieno[2,3-b]pyridin-2-yl)-3-piperidinol;
1,1,1-trifluoro-2-(6-(2-methyl-2H-pyrazolo[3,4-b]pyridin-5-yl)thieno[2,3-b]pyridin-2-yl)-2-butanol;
2-methoxy-1-(6-(2-methyl-2H-pyrazolo[3,4-b]pyridin-5-yl)thieno[2,3-b]pyridin-2-yl)ethanol;
4-(6-(2-methyl-2H-pyrazolo[3,4-b]pyridin-5-yl)thieno[2,3-b]pyridin-2-yl)tetrahydro-2H-thiopyran-4-ol 1,1-dioxide;
2-(6-(2-methyl-2H-pyrazolo[3,4-b]pyridin-5-yl)thieno[2,3-b]pyridin-2-yl)-2-propanol;
2-(6-(2-methyl-2H-pyrazolo[3,4-b]pyridin-5-yl)thieno[2,3-b]pyridin-2-yl)-2-butanol;
1-cyclopropyl-1-(6-(2-methyl-2H-pyrazolo[3,4-b]pyridin-5-yl)thieno[2,3-b]pyridin-2-yl)ethanol;
1-methyl-4-(6-(2-methyl-2H-pyrazolo[3,4-b]pyridin-5-yl)thieno[2,3-b]pyridin-2-yl)-4-piperidinol;
1-methoxy-2-(6-(2-methyl-2H-pyrazolo[3,4-b]pyridin-5-yl)thieno[2,3-b]pyridin-2-yl)-2-propanol;
3,3-difluoro-1-(6-(2-methyl-2H-pyrazolo[3,4-b]pyridin-5-yl)thieno[2,3-b]pyridin-2-yl)cyclobutanol;
1-methyl-3-(6-(2-methyl-2H-pyrazolo[3,4-b]pyridin-5-yl)thieno[2,3-b]pyridin-2-yl)-3-pyrrolidinol;
(1-methyl-4-piperidinyl)(6-(2-methyl-2H-pyrazolo[3,4-b]pyridin-5-yl)thieno[2,3-b]pyridin-2-yl)methanol;
(6-(1-methyl-1H-indazol-5-yl)thieno[2,3-b]pyridin-2-yl)(tetrahydro-2H-pyran-4-yl)methanol;
(6-(1-methyl-1H-pyrazolo[3,4-b]pyridin-5-yl)thieno[2,3-b]pyridin-2-yl)(tetrahydro-2H-pyran-4-yl)methanol;
1,1,1-trifluoro-4-methoxy-2-(6-(2-methyl-2H-pyrazolo[3,4-b]pyridin-5-yl)thieno[2,3-b]pyridin-2-yl)-2-butanol;
(6-(1H-indazol-5-yl)thieno[2,3-b]pyridin-2-yl)(tetrahydro-2H-pyran-4-yl)methanol;
(6-(6-methyl-1H-indazol-5-yl)thieno[2,3-b]pyridin-2-yl)(tetrahydro-2H-pyran-4-yl)methanol;
1,1,1-trifluoro-2-(6-(2-methyl-2H-pyrazolo[3,4-b]pyridin-5-yl)thieno[2,3-b]pyridin-2-yl)-2-propanol;
(6-(2-methyl-2H-[1,2,3]triazolo[4,5-b]pyridin-6-yl)thieno[2,3-b]pyridin-2-yl)(tetrahydro-2H-pyran-4-yl)methanol;
(6-(3-methyl-1H-indazol-5-yl)thieno[2,3-b]pyridin-2-yl)(tetrahydro-2H-pyran-4-yl)methanol;
4-methoxy-2-(6-(2-methyl-2H-pyrazolo[3,4-b]pyridin-5-yl)thieno[2,3-b]pyridin-2-yl)-2-butanol;
(6-(3-methyl-3H-[1,2,3]triazolo[4,5-b]pyridin-6-yl)thieno[2,3-b]pyridin-2-yl)(tetrahydro-2H-pyran-4-yl)methanol;
(1-methyl-3-azetidinyl)(6-(2-methyl-2H-pyrazolo[3,4-b]pyridin-5-yl)thieno[2,3-b]pyridin-2-yl)methanol;
(6-(1,2-benzoxazol-5-yl)thieno[2,3-b]pyridin-2-yl)(tetrahydro-2H-pyran-4-yl)methanol;
tetrahydro-2H-pyran-4-yl(6-(3H-[1,2,3]triazolo[4,5-b]pyridin-6-yl)thieno[2,3-b]pyridin-2-yl)methanol;
(1-methyl-2-azetidinyl)(6-(2-methyl-2H-pyrazolo[3,4-b]pyridin-5-yl)thieno[2,3-b]pyridin-2-yl)methanol;
(3,3-difluorocyclobutyl)(6-(2-methyl-1,3-benzoxazol-5-yl)thieno[2,3-b]pyridin-2-yl)methanol;
(3,3-difluorocyclobutyl)(6-(1H-pyrazolo[3,4-b]pyridin-5-yl)thieno[2,3-b]pyridin-2-yl)methanol;
(3,3-difluorocyclobutyl)(6-(2-methylimidazo[1,2-a]pyridin-6-yl)thieno[2,3-b]pyridin-2-yl)methanol;
6-(2-((3,3-difluorocyclobutyl)(hydroxy)methyl)thieno[2,3-b]pyridin-6-yl)-3-methyl-4(3H)-pyrimidinone;
6-(2-((3,3-difluorocyclobutyl)(hydroxy)methyl)thieno[2,3-b]pyridin-6-yl)-2-methyl-1(2H)-isoquinolinone;
(3,3-difluorocyclobutyl)(6-(2-methyl-1,3-benzoxazol-6-yl)thieno[2,3-b]pyridin-2-yl)methanol;
(3,3-difluorocyclobutyl)(6-(6-quinoxalinyl)thieno[2,3-b]pyridin-2-yl)methanol;
(3,3-difluorocyclobutyl)(6-(2-ethyl-2H-pyrazolo[3,4-b]pyridin-5-yl)thieno[2,3-b]pyridin-2-yl)methanol;
1-(6-(2-methyl-2H-pyrazolo[3,4-b]pyridin-5-yl)-1-benzothiophen-2-yl)-1-butanol;
(3,3-difluorocyclobutyl)(6-(2-methylimidazo[1,2-a]pyridin-7-yl)thieno[2,3-b]pyridin-2-yl)methanol;
(3,3-difluorocyclobutyl)(6-(2-(2-propanyl)-2H-indazol-5-yl)thieno[2,3-b]pyridin-2-yl)methanol;
(3,3-difluorocyclobutyl)(6-(5-pyrimidinyl)thieno[2,3-b]pyridin-2-yl)methanol;
cyclopentyl(6-(2-methyl-2H-pyrazolo[3,4-b]pyridin-5-yl)-1-benzothiophen-2-yl)methanol;
3-methoxy-1-(6-(2-methyl-2H-pyrazolo[3,4-b]pyridin-5-yl)-1-benzothiophen-2-yl)-1-propanol;
4,4,4-trifluoro-1-(6-(2-methyl-2H-pyrazolo[3,4-b]pyridin-5-yl)-1-benzothiophen-2-yl)-1-butanol;
(3,3-difluorocyclobutyl)(6-(2-methyl-2H-pyrazolo[3,4-b]pyridin-5-yl)-1-benzothiophen-2-yl)methanol;
(6-(2-methyl-2H-pyrazolo[3,4-b]pyridin-5-yl)thieno[2,3-b]pyridin-2-yl)(1-methyl-3-pyrrolidinyl)methanol;
(1-methyl-3-piperidinyl)(6-(2-methyl-2H-pyrazolo[3,4-b]pyridin-5-yl)thieno[2,3-b]pyridin-2-yl)methanol;
(3,3-difluorocyclobutyl)(6-(2-(2-propanyl)-2H-pyrazolo[3,4-b]pyridin-5-yl)thieno[2,3-b]pyridin-2-yl)methanol;
(3,3-difluorocyclobutyl)(6-(1-(2-propanyl)-1H-pyrazolo[3,4-b]pyridin-5-yl)thieno[2,3-b]pyridin-2-yl)methanol;
(6-(2-methyl-2H-pyrazolo[3,4-b]pyridin-5-yl)thieno[2,3-b]pyridin-2-yl)(tetrahydro-2H-pyran-4-yl)methan-d-ol; or
(3-amino-6-(5-pyrimidinyl)thieno[2,3-b]pyridin-2-yl)(3,3-difluorocyclobutyl)methanol.
   A209. The compound or salt of Embodiment A1, wherein the compound is:

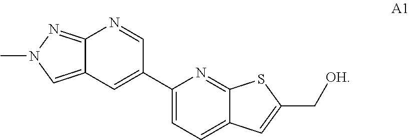 [SMILES: Cn1cc2cc(-c3ccc4cc(CO)sc4n3)cnc2n1]

A210. The compound or salt of Embodiment A1, wherein the compound is:

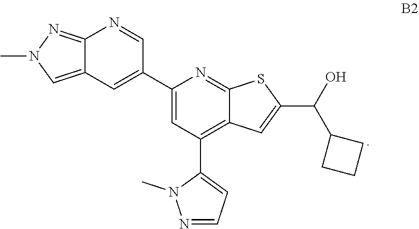 [SMILES: Cn1cc2cc(-c3cc(-c4ccnn4C)c4cc(C(O)C5CCC5)sc4n3)cnc2n1]

A211. The compound or salt of Embodiment A1, wherein the compound is:

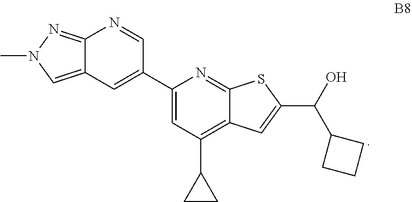 [SMILES: Cn1cc2cc(-c3cc(C4CC4)c4cc(C(O)C5CCC5)sc4n3)cnc2n1]

A212. The compound or salt of Embodiment A1, wherein the compound is:

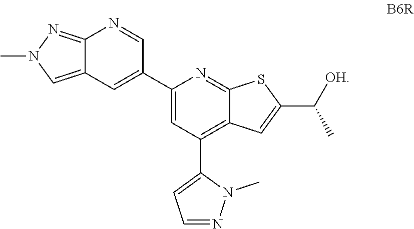 [SMILES: C[C@@H](O)c1cc2c(-c3ccnn3C)cc(-c3cnc4nn(C)cc4c3)nc2s1]

A213. The compound or salt of Embodiment A1, wherein the compound is:

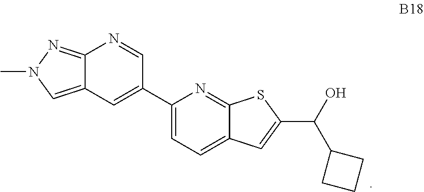 [SMILES: Cn1cc2cc(-c3ccc4cc(C(O)C5CCC5)sc4n3)cnc2n1]

A214. The compound or salt of Embodiment A1, wherein the compound is:

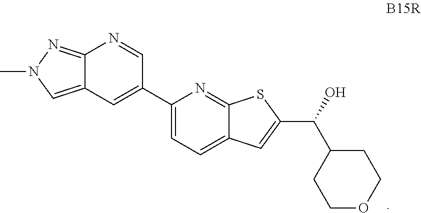 [SMILES: Cn1cc2cc(-c3ccc4cc([C@H](O)C5CCOCC5)sc4n3)cnc2n1]

A215. The compound or salt of Embodiment A1, wherein the compound is:

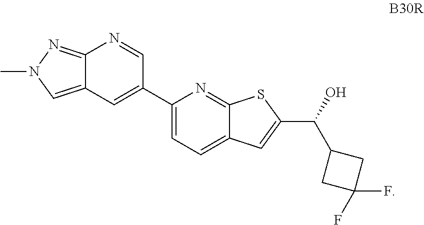 [SMILES: Cn1cc2cc(-c3ccc4cc([C@H](O)C5CC(F)(F)C5)sc4n3)cnc2n1]

A216. The compound or salt of Embodiment A1, wherein the compound is:

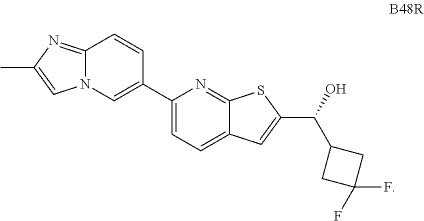 [SMILES: Cc1cn2cc(-c3ccc4cc([C@H](O)C5CC(F)(F)C5)sc4n3)ccc2n1]

A217. The compound or salt of Embodiment A1, wherein the compound is:

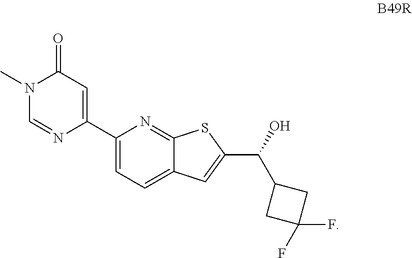 [SMILES: Cn1cnc(-c2ccc3cc([C@H](O)C4CC(F)(F)C4)sc3n2)cc1=O]

A218. The compound or salt of Embodiment A1, wherein the compound is:

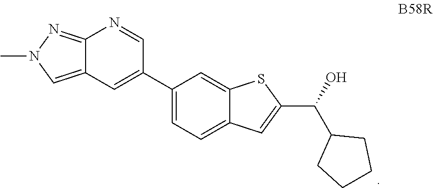 [SMILES: Cn1cc2cc(-c3ccc4cc([C@H](O)C5CCCC5)sc4c3)cnc2n1]

A219. The compound or salt of Embodiment A1, wherein the compound is:

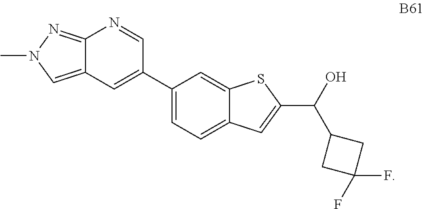 [SMILES: Cn1cc2cc(-c3ccc4cc(C(O)C5CC(F)(F)C5)sc4c3)cnc2n1]

A220. The compound or salt of Embodiment A1, wherein the compound is:

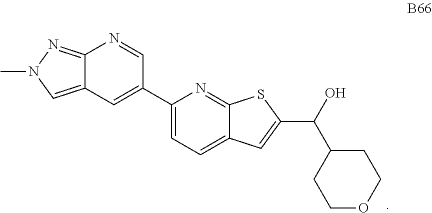 [SMILES: Cn1cc2cc(-c3ccc4cc(C(O)C5CCOCC5)sc4n3)cnc2n1]

A221. The compound or salt of Embodiment A1, wherein the compound is:

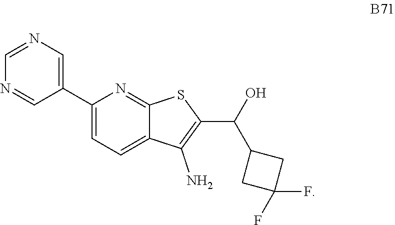 [SMILES: Nc1c(C(O)C2CC(F)(F)C2)sc2nc(-c3cncnc3)ccc12]

A222. The compound or salt of Embodiment A1, wherein the compound is:

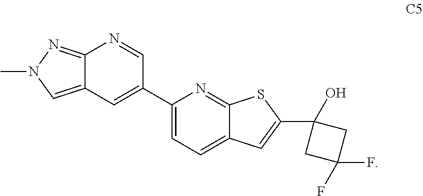 [SMILES: Cn1cc2cc(-c3ccc4cc(C5(O)CC(F)(F)C5)sc4n3)cnc2n1]

A223. A pharmaceutical composition comprising the compound according to any one of Embodiments A1 to A222, and a pharmaceutically acceptable excipient.
A224. A compound or salt according to any one of Embodiments A l to A222, or the pharmaceutical composition of Embodiment A223 for use as a medicament.
A225. A compound or salt according to any one of Embodiments A1 to A222, or the pharmaceutical composition of Embodiment A223 for use in treating a 15-PGDH mediated disease or disorder.
A226. A compound or salt according to any one of Embodiments A l to A222, or the pharmaceutical composition of Embodiment A223 for use in treating inflammatory bowel disease.
A227. A compound or salt according to any one of Embodiments A l to A222, or the pharmaceutical composition of Embodiment A223 for use in treating ulcerative colitis.
A228. A compound or salt according to any one of Embodiments A1 to A222, or the pharmaceutical composition of Embodiment A223 for use in treating Crohn's disease.
A229. A compound or salt according to any one of Embodiments A1 to A222, or the pharmaceutical composition of Embodiment A223 for use in treating a fibrotic disease, disorder or condition.
A230. The compound, salt, or composition of Embodiment A229, wherein the fibrotic disease, disorder, or condition is characterized, in whole or in part, by the excess production of fibrous material, including excess production of fibrotic material within the extracellular matrix, or the replacement of normal tissue elements by abnormal, non-functional, and/or excessive accumulation of matrix-associated components.
A231. The compound, salt, or composition of Embodiment A229 or A230, wherein the fibrotic disease, disorder, or condition is systemic sclerosis, multifocal fibrosclerosis, nephrogenic systemic fibrosis, kidney fibrosis, glomerular sclerosis, renal tubulointerstitial fibrosis, progressive renal disease or diabetic nephropathy, cardiac fibrosis, pulmonary fibrosis, glomerulosclerosis pulmonary fibrosis, idiopathic pulmonary fibrosis, silicosis, asbestosis, interstitial lung disease, interstitial fibrotic lung disease, chemotherapy/radiation induced pulmonary fibrosis, endomyocardial fibrosis, deltoid fibrosis, pancreatitis, general fibrosis syndrome characterized by replacement of normal muscle tissue by fibrous tissue in varying degrees, retroperitoneal fibrosis, liver fibrosis, liver cirrhosis, chronic renal failure; myelofibrosis, bone marrow fibrosis, acute fibrosis, or organ specific fibrosis.
A232. The compound, salt, or composition of Embodiment A229 or A230, wherein the fibrotic disease, disorder, or condition is idiopathic pulmonary fibrosis.
A233. The compound, salt, or composition of Embodiment A229 or A230, wherein the fibrotic disease, disorder, or condition is kidney fibrosis.
A234. The compound, salt, or composition of Embodiment A229 or A230, wherein the fibrotic disease, disorder, or condition is liver fibrosis.
A235. A compound or salt according to any one of Embodiments A1 to A222, or the pharmaceutical composition of Embodiment A223 for use in treating one or more of atherosclerotic cardiovascular disease, autoimmune disease, intestinal ischemia, ischemia, ischemic brain disease, ischemic heart disease, ischemic peripheral vascular disease, ischemic placenta, ischemic renal disease, ischemic vascular disease, ischemic-reperfusion injury, limb ischemia, lower extremity ischemia, myocardial ischemia, organ ischemia, peripheral ischemia, tissue ischemia, transient ischemic attack (TIA), and wounds to tissues or organs.
A236. A method of treating a 15-PGDH mediated disease, disorder, or condition in a subject in need thereof, the method comprising:
administering to the subject a therapeutically acceptable amount of a compound or salt according to any one of Embodiments A l to A222, or the composition of Embodiment A223.
A237. A method of treating intestinal, gastrointestinal, or bowel disorders in a subject in need thereof, the method comprising:
administering to the subject a therapeutically acceptable amount of a compound or salt according to any one of Embodiments A l to A222, or the composition of Embodiment A223.
A238. The method of Embodiment A236 or A237, wherein the disease or disorder comprises at least one of ulcerative colitis, inflammatory bowel disease, and Crohn's disease.
A239. A method of manufacturing the compound or salt of Embodiment A1, the method comprising:
performing a Suzuki coupling between a compound of Formula (I-int-1)

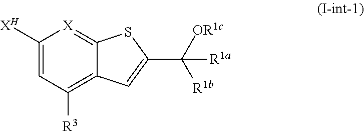

with a compound of Formula (R2-B):

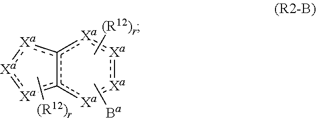

where
X^{H} is a —Br or —Cl;
B^{a} is a boronic acid group or a boronic ester group;
[symbol] indicates a double bond or single bond may be present;
each instance of X^{a} independently is nitrogen, oxygen, or carbon;
each instance of r, where present, is an integer selected from 0, 1, 2, 3, 4, or 5, wherein where two instances of r occur on the same R² group, the total of both r values combined does not exceed 5;
each instance of R¹², when present and attached to an N ring member, independently is C₁₋₆alkyl, C₁₋₆haloalkyl, C₁₋₆alkylene-OH, or C₁₋₆alkylene-O—C₁₋₆alkyl; and
each instance of R¹², when present and attached to a C ring member, independently is —OH, oxo, C₁₋₆ alkyl, C₁₋₆ haloalkyl, C₁₋₆ alkoxy, C₁₋₆alkylene-OH, or C₁₋₆alkylene-O—C₁₋₆alkyl;
wherein a —N=ring nitrogen can be bonded to an R¹² to provide a cation.
A240. A method of manufacturing the compound or salt of Embodiment A1, the method comprising using a compound of Formula (I-int) as an intermediate:

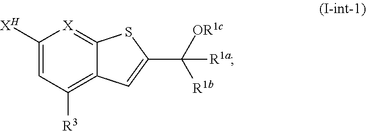

wherein
X^{H} is a —Cl, —Br, -OTf, -OTs, or -OMs;
R^{1a} is —H, C₁₋₆ alkyl, or C₁₋₆ haloalkyl;
   wherein R^{1a} may be unsubstituted or substituted with one or more substituents, each of which independently is halogen, —OH, —N(R^{b})₂, C₁₋₃ alkyl, or C₁₋₃ alkoxy;
R^{1b} is —H, C₁₋₆ alkyl, C₁₋₆ haloalkyl, C₃₋₁₀ cycloalkyl, or heterocyclyl having 3 to 10 ring members;
   wherein R^{1b} may be unsubstituted or substituted with one or more substituents, each of which independently is halogen, —OH, —N(R^{b})₂, C₁₋₃ alkyl, or C₁₋₃ alkoxy;
or, alternatively, R^{1a} and R^{1b} together form a C₃₋₁₀ cycloalkyl, C₃₋₁₀ cycloalkenyl, or heterocyclyl having 3 to 10 ring members;
   wherein the R^{1a} and R^{1b} cycle may be unsubstituted or substituted with one or more substituents, each of which independently is halogen, —OH, oxo, —N(R^{b})₂, C₁₋₃ alkyl, C₁₋₃alkoxy, or C₁₋₆ heteroalkyl;
R^{1c} is —H, C₁₋₆ alkyl, or C₃₋₆ cycloalkyl;
   wherein R^{1c} may be unsubstituted or substituted with one or more substituents, each of which independently is halogen, —OH, —N(R^{b})₂, C₁₋₃ alkyl, C₁₋₃ alkoxy, or C₁₋₆heteroalkyl;
R³ is —H, C₁₋₆ alkyl, C₁₋₆ haloalkyl, C₃₋₆ cycloalkyl, amino, C₁₋₄alkyl-amino, di-C₁₋₄alkyl-amino, C₆₋₁₀ aryl, heteroaryl having 5 to 10 ring members, or heterocyclyl having 5 to 10 ring members;
   wherein R³ may be unsubstituted or substituted with one or more substituents, each of which independently is halogen, —OH, oxo, —N(R^{b})₂, C₁₋₃ alkyl, C₁₋₃ haloalkyl, C₁₋₃ alkoxy, C₁₋₃ alkylene-C₁₋₃ alkoxy, C₁₋₆ heteroalkyl, or two R³ substituents together form a C₃₋₆ carbocyclyl or a heterocyclyl having 3 to 6 ring members;
where each instance of R, where present, independently is —H or C₁₋₆ alkyl; or, alternatively, two R^{b} substituents together form a heterocyclyl having 3 to 6 ring members;
   wherein R^{b} may be unsubstituted or substituted with one or more substituents, each of which independently is —F, —Cl, —OH, C₁₋₃ alkoxy.
A241. A method of manufacturing the compound or salt of Embodiment A1, the method comprising using a compound of Formula (I-int-a) as an intermediate:

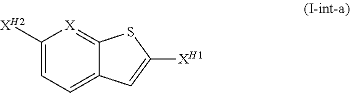

or a pharmaceutically acceptable salt of said compound;
   where
X is N or CH;
X^{H1} is —Cl, —Br, or —I; and
X^{H2} is —Cl, —Br, -OTf, -OTs, or -OMs.
A242. A method of manufacturing the compound or salt of Embodiment A1, the method comprising using a compound of Formula (I-int-b) as an intermediate:

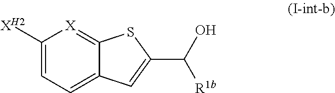

or a pharmaceutically acceptable salt of said compound;
   where
X is N or CH;
X^{H2}—Cl, —Br, -OTf, -OTs, or -OMs; and
R^{1b} is —H, C₁₋₆ alkyl, C₁₋₆ haloalkyl, C₃₋₁₀ cycloalkyl, or heterocyclyl having 3 to 10 ring members;
wherein R^{1b} may be unsubstituted or substituted with one or more substituents, each of which independently is halogen, —OH, —N(R^{b})₂, C₁₋₃ alkyl, or C₁₋₃ alkoxy;
where each instance of R^{b}, where present, independently is —H or C₁₋₆ alkyl; or, alternatively, two R^{b} substituents together form a heterocyclyl having 3 to 6 ring members; and
wherein R^{b} may be unsubstituted or substituted with one or more substituents, each of which independently is —F, —Cl, —OH, C₁₋₃ alkoxy.
A243. A method of manufacturing the compound or salt of Embodiment A1, the method comprising using a compound of Formula (I-int-c) as an intermediate:

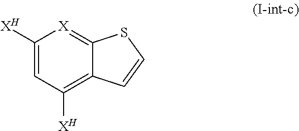

or a pharmaceutically acceptable salt of said compound;
   where
X is N or CH; and
each instance of X^{H} independently is —C₁ or —Br.
A244. A method of manufacturing the compound or salt of Embodiment A1, the method comprising using a compound of Formula (I-int-d) as an intermediate:

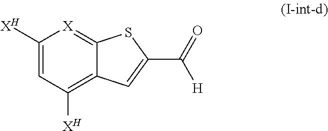

or a pharmaceutically acceptable salt of said compound;
   where
X is N or CH; and
each instance of X^{H} independently is —C₁ or —Br.
A245. A method of manufacturing the compound or salt of Embodiment A1, the method comprising using a compound of Formula (I-int-e) as an intermediate:

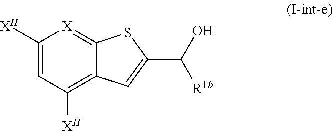

where
X is N or CH;
each instance of X^{H} independently is C₁ or —Br; and
R^{1b} is —H, C₁₋₆ alkyl, C₁₋₆ haloalkyl, C₃₋₁₀ cycloalkyl, or heterocyclyl having 3 to 10 ring members;
wherein R^{1b} may be unsubstituted or substituted with one or more substituents, each of which independently is halogen, —OH, —N(R^{b})₂, C₁₋₃ alkyl, or C₁₋₃ alkoxy;
where each instance of R^{b}, where present, independently is —H or C₁₋₆ alkyl; or, alternatively, two R^{b} substituents together form a heterocyclyl having 3 to 6 ring members; and
wherein R^{b} may be unsubstituted or substituted with one or more substituents, each of which independently is —F, —Cl, —OH, C₁₋₃ alkoxy.
A246. A method of manufacturing the compound or salt of Embodiment A1, the method comprising using a compound of Formula (I-int-f) as an intermediate:

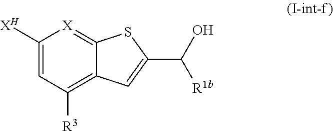

where
X is N or CH;
X^{H} is a halogen; and
R^{1b} is —H, C₁₋₆ alkyl, C₁₋₆ haloalkyl, C₃₋₁₀ cycloalkyl, or heterocyclyl having 3 to 10 ring members (wherein R^{1b} may be unsubstituted or substituted as disclosed elsewhere herein);
R³ is —H, C₁₋₆ alkyl, C₁₋₆ haloalkyl, C₃₋₆ cycloalkyl, amino, C₁₋₄alkyl-amino, di-C₁₋₄alkyl-amino, C₆₋₁₀ aryl, heteroaryl having 5 to 10 ring members, or heterocyclyl having 5 to 10 ring members (wherein R^{1b} may be unsubstituted or substituted as disclosed elsewhere herein).

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### EXAMPLES

This section provides specific examples of compounds of Formula (A) and Formula (I) and methods of making the same. If the chemical structure and chemical name conflict, the chemical structure is determinative of the identity of the compound.

TABLE A
List of Abbreviations

| | |
|---|---|
| Ac | acetyl |
| MeCN or ACN | acetonitrile |
| AcOH | acetic acid |
| aq or aq. | aqueous |
| BOC or Boc | tert-butyloxycarbonyl |
| B₂pin₂ | 4,4,4′,4′,5,5,5′,5′-octamethyl-2,2′-bi(1,3,2- |
| | dioxaborolane) |
| cataCXium | [(di(1-adamantyl)-butylphosphine)-2-(2′-amino-1,1′- |
| A Pd G3 | biphenyl)]palladium(II) methanesulfonate |
| Cy | cyclohexyl |
| DABCO | 1,4-diazabicyclo[2.2.2]octane |
| DAST | diethylaminosulfur trifluoride |
| DCM | dichloromethane |
| DEA | diethylamine |
| DMF | N,N-dimethylformamide |
| DMSO | dimethyl sulfoxide |
| equiv. | equivalent |
| ESI or ES | electrospray ionization |
| Et | ethyl |
| EtOAc | ethyl acetate |
| EtOH | ethanol |
| g or gr | gram(s) |
| h | hour(s) |
| HATU | hexafluorophosphate azabenzotriazole tetramethyl |
| | uronium |
| HBpin | 4,4,5,5-tetramethyl-1,3,2-dioxaborolane |
| HPLC | high pressure liquid chromatography |
| IBX | 2-iodoxybenzoic acid |
| iPr | iso-propyl |
| iPrOH or IPA | iso-propanol |
| iPAm or IPAm | iso-propylamine |
| KOAc | potassium acetate |
| LC MS, LCMS, | liquid chromatography mass spectroscopy |
| LC-MS or LC/MS | |
| LDA | lithium diisopropylamide |
| m/z | mass divided by charge |
| Me | methyl |
| MeI | iodomethane |
| MeOH | methanol |
| mg | milligrams |
| min | minutes |
| mL | milliliters |
| MS | mass spectra |
| MTBE | methyl tert-butyl ether |
| NaHMDS | sodium hexamethyldisilazide |
| NMR | nuclear magnetic resonance |
| PCy₃ | tricyclohexylphosphine |
| Pd(amphos)Cl₂ | bis(di-tert-butyl(4-dimethylamino- |
| | phenyl)phosphine)dichloropalladium(II) |
| Pd/C | palladium on carbon |
| Pd₂(dba)₃ | tris(dibenzylideneacetone)dipalladium(0) |
| Pd(dppf)Cl₂•DCM, | [1,1′- |
| Pd(dppf)Cl₂ | bis(diphenylphosphino)ferrocene]dichloro- |
| | palladium(II), complex with dichloromethane |
| Pd(PPh₃)₄ | tetrakis(triphenylphosphine)palladium(0) |
| pet. ether | Petroleum ether |
| Ph | phenyl |
| Pin | pinacolato |
| RP-HPLC | reverse phase high pressure liquid chromatography |
| RT or rt or r.t. | room temperature |
| sat. or satd. | saturated |
| SFC | supercritical fluid chromatography |
| SPhos Pd G3 | (2-dicyclohexylphosphino-2′,6′- |
| | dimethoxybiphenyl) [2-(2′-amino-1,1′- |
| | biphenyl)]palladium(II) methanesulfonate |
| TBAF | tetra-n-butylammonium fluoride |
| TEA or Et₃N | triethylamine |
| temp | temperature |
| TFA | trifluoroacetic acid |
| THF | tetrahydrofuran |
| UV | ultraviolet |
| XPhos | 2-dicyclohexylphosphino-2′,4′,6′-triisopropyl-1,1′- |
| | biphenyl |

### Section 1: Intermediates

Provided in this section is the synthesis of various intermediates used to prepare compounds of Formula (A) or Formula (I). All starting materials are either commercially available from Fisher Scientific, Enamine, or similar vendors, unless otherwise noted, or known in the art and may be synthesized by employing known procedures using ordinary skill.

### Intermediate 1: 2-methyl-5-(4,4,5,5-tetramethyl-1,3,2-dioxaborolan-2-yl)-2H-pyrazolo[3,4-b]pyridine

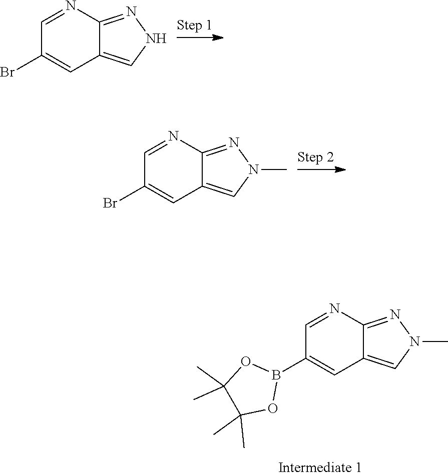

Step 1: 5-bromo-2-methyl-2H-pyrazolo[3,4-b]pyridine. To a mixture of 5-bromo-2H-pyrazolo[3,4-b]pyridine (50.0 g, 252 mmol) in THF (1600 mL) was added NaHMDS (379 mL, 379 mmol) at 0° C. After stirring for 0.5 h., Mel (47.4 mL, 757 mmol) was added at 0° C. The mixture was warmed to 25° C. and stirred for 4 h. The reaction mixture was quenched by addition of H₂O (300 mL) at 0° C., then diluted with H₂O (200 mL), extracted with EtOAc (3×150 mL), washed with brine (150 mL), dried over Na₂SO₄, filtered and concentrated in vacuo. The crude mixture of isomers was absorbed onto a plug of silica gel and purified by column chromatography, eluting with a gradient of 0% to 70% EtOAc in pet. ether, to provide 5-bromo-2-methyl-2H-pyrazolo[3,4-b]pyridine (8.00 g, 37.7 mmol, 75% yield). m z (ESI): 212.0 (M+H)⁺. ¹H NMR (400 MHz, DMSO-d₆) δ 8.59 (d, J 2.4 Hz, 1H), 8.42 (s, 1H) 8.51 (d, J 2.4 Hz, 1H), 4.21 (s, 3H).

Step 2: 2-methyl-5-(4,4,5,5-tetramethyl-1,3,2-dioxaborolan-2-yl)-2H-pyrazolo[3,4-b]pyridine. To a solution of 5-bromo-2-methyl-2H-pyrazolo[3,4-b]pyridine (20 g, 94 mmol) in 1,4-dioxane (300 mL) was added KOAc (13.86 g, 141 mmol), B₂pin₂ (28.7 g, 113 mmol) and Pd(dppf)Cl₂-DCM adduct (3.85 g, 4.72 mmol). The mixture was stirred at 100° C. for 12 h under an atmosphere of N₂. The mixture was filtered through a celite plug, and the filtrate was concentrated in vacuo. The reaction mixture was diluted with EtOAc (50 mL)/pet. ether (300 mL) and stirred at 20° C. for 15 min. The mixture was filtered, and the filter cake was washed with pet. ether (3×300 mL). The filtrate was concentrated in vacuo, and the solid was further dried under high vacuum to provide Intermediate 1 (24 g, 93 mmol, 98% yield). m/z (ESI): 178.1 (M-pinacol+2OH)⁺. ¹H NMR (400 MHz, DMSO-d₆) δ 8.72 (d, J 1.8 Hz, 1H), 8.53 (d, J 1.8 Hz, 1H), 8.47 (s, 1H), 4.21 (s, 3H), 1.32 (s, 12H).

### Intermediate 2: 2-isopropyl-5-(4,4,5,5-tetramethyl-1,3,2-dioxaborolan-2-yl)-2H-pyrazolo[3,4-b]pyridine

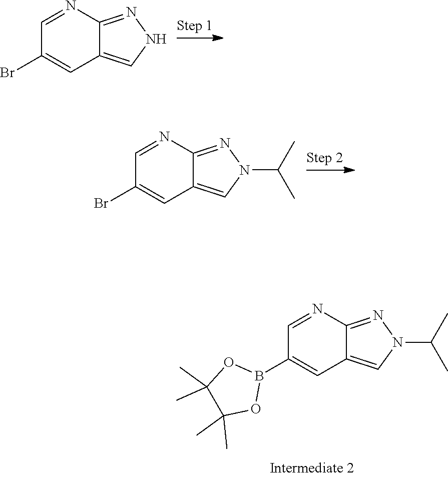 [SMILES: Brc1cnc2n[nH]cc2c1 ; CC(C)n1cc2cc(Br)cnc2n1 ; CC(C)n1cc2cc(B3OC(C)(C)C(C)(C)O3)cnc2n1]

Step 1: A similar protocol as Intermediate 1 was used, with 5-bromo-2H-pyrazolo[3,4-b]pyridine as starting material. m/z (ESI): 240.0 (M+H)⁺.

Step 2: 2-isopropyl-5-(4,4,5,5-tetramethyl-1,3,2-dioxaborolan-2-yl)-2H-pyrazolo[3,4-b]pyridine. A similar protocol as Intermediate 1 was used, with 5-bromo-2-isopropyl-2H-pyrazolo[3,4-b]pyridine as starting material. m/z (ESI): 206.2 (M-pinacol+2OH)⁺.

### Intermediate 3: 1-isopropyl-5-(4,4,5,5-tetramethyl-1,3,2-dioxaborolan-2-yl)-1H-pyrazolo[3,4-b]pyridine

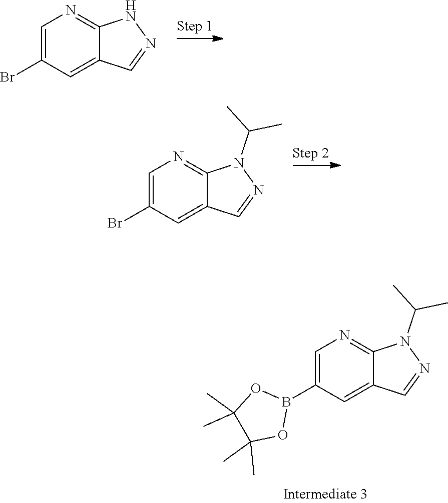 [SMILES: Brc1cnc2[nH]ncc2c1 ; CC(C)n1ncc2cc(Br)cnc21 ; CC(C)n1ncc2cc(B3OC(C)(C)C(C)(C)O3)cnc21]

Step 1: A similar protocol as Intermediate 1 was used, with 5-bromo-1H-pyrazolo[3,4-b]pyridine as starting material. m/z (ESI): 240.0 (M+H)⁺.

Step 2: 1-isopropyl-5-(4,4,5,5-tetramethyl-1,3,2-dioxaborolan-2-yl)-1H-pyrazolo[3,4-b]pyridine. A similar protocol as Intermediate 1 was used, with 5-bromo-1-isopropyl-1H-pyrazolo[3,4-b]pyridine as starting material. m/z (ESI): 206.8 (M-pinacol+20H)+. Intermediate 4: 6-(4,4,5,5-tetramethyl-1,3,2-dioxaborolan-2-yl)-1-((2-(trimethylsilyl)ethoxy)methyl)-1H-[1,2,3]triazolo[4,5-b]pyridine

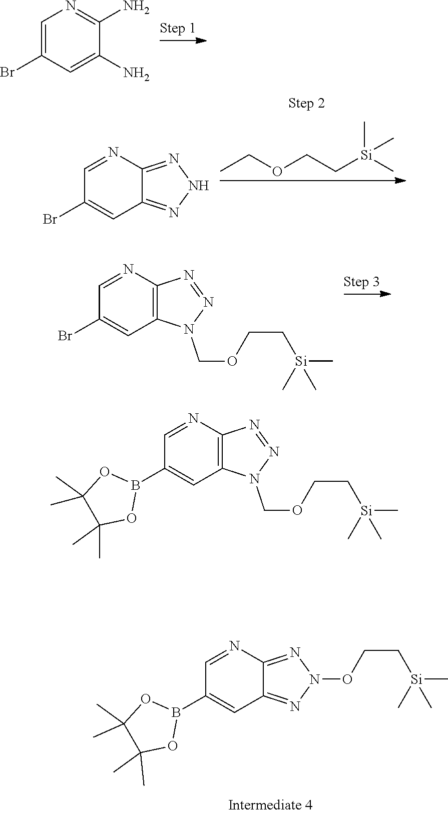

Step 1: 6-bromo-2H-[1,2,3]triazolo[4,5-b]pyridine. To a solution of 1 N H₂SO₄ (150 mL) in H₂O (500 mL) was added 5-bromo-2,3-dihydropyridine-2,3-diamine (10 g, 52.6 mmol). The mixture was cooled to 0° C. and NaNO₂ (4.25 g, 61.6 mmol) in H₂O (120 mL) was added. Then, the mixture was stirred at 25° C. for 12 h under an atmosphere of N₂. The reaction mixture was poured into H₂O (80 mL), extracted with 2Me-THF (3×60 mL), washed with brine (50 mL), dried over Na₂SO₄, filtered and concentrated in vacuo to obtain the residue. The resulting residue was stirred with MTBE (50 mL) at 25° C. for 0.5 h. The suspension was filtered, and the filter cake was dried under reduced pressure to provide 6-bromo-2H-[1,2,3]triazolo[4,5-b]pyridine (8.00 g, 40.2 mmol, 76% yield). m/z (ESI): 198.9 (M+H)⁺. ¹H NMR (400 MHz, DMSO-d₆) δ 8.78-8.79 (m, 2H).

Step 2: 6-bromo-1-((2-(trimethylsilyl)ethoxy)methyl)-1H-[1,2,3]triazolo[4,5-b]pyridine. To a solution of 6-bromo-1H-[1,2,3]triazolo[4,5-b]pyridine (lg, 5.02 mmol) in THF (10 mL) at 0° C. was added NaH (0.181 g, 7.54 mmol). The mixture was stirred at 0° C. for 30 min. Then, (2-ethoxyethyl)trimethylsilyl chloride (1.103 g, 6.62 mmol) was added dropwise at 0° C., and stirring was continued for 1 h. The reaction mixture was diluted with H₂O (40 mL) and sat. aq. NH₄Cl (30 mL), extracted with EtOAc (30 mL×3), washed with brine (30 mL), dried over Na₂SO₄, filtered and the filtrate was concentrated in vacuo. The crude material was absorbed onto a plug of silica gel and purified by column chromatography, eluting with a gradient of 0% to 55% EtOAc in pet. ether, to provide 6-bromo-1-((2-(trimethylsilyl)ethoxy)methyl)-1H-[1,2,3]triazolo[4,5-b]pyridine (650 mg, 1.97 mmol, 39% yield). m/z (ESI): 328.9 (M+H)⁺. ¹H NMR (400 MHz, DMSO-d₆) δ 9.02 (d, J 2.0 Hz, 1H), 8.92 (d, J 2.0 Hz, 1H), 6.04 (s, 2H), 3.67 (t, J 8.0 Hz, 2H), 0.84 (t, J 8.0 Hz, 2H), —0.11 (s, 9H).

Step 3: 6-(4,4,5,5-tetramethyl-1,3,2-dioxaborolan-2-yl)-1-((2-(trimethylsilyl)ethoxy)methyl)-1H-[1,2,3]triazolo[4,5-b]pyridine & 6-(4,4,5,5-tetramethyl-1,3,2-dioxaborolan-2-yl)-2-((2-(trimethylsilyl)ethoxy)methyl)-2H-[1,2,3]triazolo[4,5-b]pyridine. To a solution of B₂pin₂ (93.0 mg, 0.36 mmol) and 6-bromo-1-((2-(trimethylsilyl)ethoxy)methyl)-1H-[1,2,3]triazolo[4,5-b]pyridine (100 mg, 0.30 mmol) in 1,4-dioxane (1 mL) was added KOAc (44.7 mg, 0.46 mmol) and Pd(dppf)Cl₂-DCM adduct (12.6 mg, 0.015 mmol). The mixture was degassed with N₂, then stirred at 80° C. for 12 h. The solvent was removed in vacuo, and the mixture was used without further purification for the next step. m/z (ESI): 377.1 (M+H)⁺.

### Intermediate 5: 2-methyl-6-(4,4,5,5-tetramethyl-1,3,2-dioxaborolan-2-yl)-2H-[1,2,3]triazolo[4,5-b]pyridine

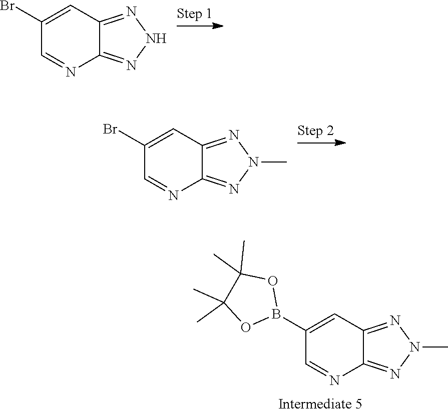

Step 1: 6-bromo-2-methyl-2H-[1,2,3]triazolo[4,5-b]pyridine. 6-bromo-2-methyl-2H-[1,2,3]triazolo[4,5-b]pyridine was prepared according to the literature procedure (Journal of Organic Chemistry (2018), 83(12), 6334-6353).

Step 2: 2-methyl-6-(4,4,5,5-tetramethyl-1,3,2-dioxaborolan-2-yl)-2H-[1,2,3]triazolo[4,5-b]pyridine. To a solution of 6-bromo-2-methyl-2H-[1,2,3]triazolo[4,5-b]pyridine (1.50 g, 7.04 mmol) in 1,4-dioxane (5 mL) was added B₂Pin₂ (3.580 g, 14.08 mmol), KOAc (1.175 g, 11.97 mmol), and Pd(dppf)Cl₂ DCM (0.575 g, 0.70 mmol) in sequence. Then the mixture was stirred at 110° C. for 12 h. The reaction mixture was diluted with H₂O (20 mL) and extracted with EtOAc (3×10 mL). The combined organic layers were washed with brine (15 mL), dried over Na₂SO₄, filtered and concentrated under reduced pressure to give a residue. The residue was purified by column chromatography (pet. ether/EtOAc=5/1 to 3/1) to afford Intermediate 5 (1.60 g, 6.15 mmol, 87% yield). m/z (ESI): 261.0 (M+H)⁺.

### Intermediate 6: (2-methylimidazo[1,2-b]pyridazin-7-yl)boronic acid

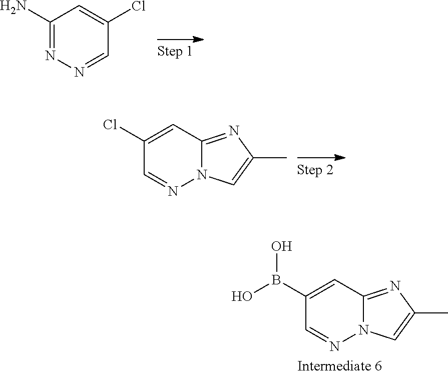

Step 1: 7-chloro-2-methylimidazo[1,2-b]pyridazine. To a stirred solution of 1-chloropropan-2-one (4.29 g, 46.3 mmol) in EtOH (40.0 mL) at rt under N₂ was added 5-chloropyridazin-3-amine (2.00 g, 15.4 mmol). The reaction mixture was stirred at 100° C. for 16 h. The reaction mixture was concentrated under reduced pressure and dried under vacuum. The residue was washed with EtOAc (200 mL×5) and filtered, and the filtrate was concentrated under reduced pressure to provide 7-chloro-2-methylimidazo[1,2-b]pyridazine (0.4 g, 2.39 mmol, 15% yield). m/z (ESI): 168.2 (M+H)⁺. ¹H NMR (400 MHz, DMSO-d₆) δ 8.56 (d, J=2.4 Hz, 1H), 8.28 (d, J=2.4 Hz, 1H), 8.11 (d, J=1.1 Hz, 1H), 2.39 (d, J=0.9 Hz, 3H).

Step 2: (2-methylimidazo[1,2-b]pyridazin-7-yl)boronic acid. A mixture of 7-chloro-2-methylimidazo[1,2-b]pyridazine (0.1 g, 0.60 mmol), potassium acetate (0.234 g, 2.39 mmol), and B₂pin₂ (0.758 g, 2.98 mmol) in 1,4-dioxane (8.00 mL) was sealed at rt, and the mixture was degassed and purged with N₂ for 5 min. Next, XPhos (0.028 g, 0.06 mmol) and Pd₂(dba)₃ (0.027 g, 0.03 mmol) were added at rt, and the reaction was heated to 100° C. for 16 h. Then, the reaction was diluted with EtOAc, filtered, and washed with EtOAc (50 mL). The filtrate was concentrated under reduced pressure to afford crude Intermediate 6, which was used without further purification. m/z (ESI): 178.2 (M+H)⁺.

### Intermediate 7: (2-methylimidazo[1,2-a]pyridin-7-yl)boronic acid

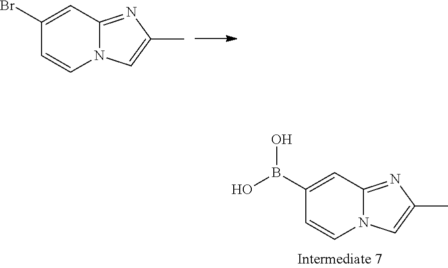

(2-methylimidazo[1,2-a]pyridin-7-yl)boronic acid. A mixture of 7-bromo-2-methylimidazo[1,2-a]pyridine (0.2 g, 0.948 mmol), potassium acetate (0.372 g, 3.79 mmol), and B₂pin₂ (1.20 g, 4.74 mmol) in 1,4-dioxane (8.00 mL) at rt was degassed and purged with N₂ for 5 min. Then, Pd(dppf)Cl₂ DCM adduct (0.077 g, 0.10 mmol) was added at rt. The reaction mass was heated to 110° C. for 5 h. Then, the reaction mass was diluted with EtOAc, filtered, and washed with EtOAc (50 mL). Next, the filtrate was concentrated under reduced pressure to afford crude Intermediate 7, which was used without further purification. m/z (ESI): 177.2 (M+H)⁺.

### Intermediate 8: 2-methyl-6-(4,4,5,5-tetramethyl-1,3,2-dioxaborolan-2-yl)imidazo[1,2-a]pyrimidine

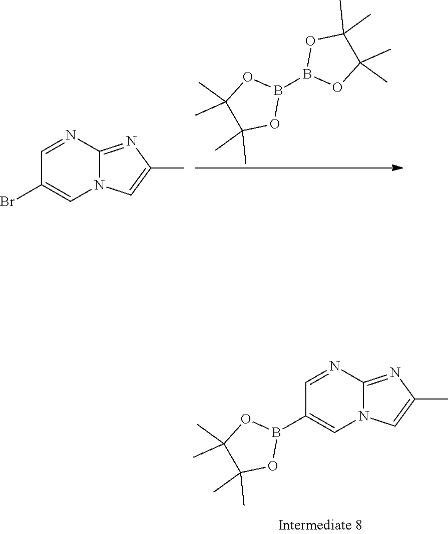

2-methyl-6-(4,4,5,5-tetramethyl-1,3,2-dioxaborolan-2-yl)imidazo[1,2-a]pyrimidine. A mixture of 6-bromo-2-methylimidazo[1,2-a]pyrimidine (50 mg, 0.236 mmol), potassium acetate (93 mg, 0.943 mmol) and B₂pin₂ (299 mg, 1.179 mmol) in 1,4-dioxane (4.00 mL)) at rt was degassed for 5 minutes. XPhos (11.24 mg, 0.024 mmol) and Pd₂(dba)₃ (10.80 mg, 0.012 mmol) were added at rt, and the reaction mass was heated to 100° C. for 16 h. The reaction mass was diluted with EtOAc (10 mL), filtered through celite and washed with EtOAc (5 mL). The filtrate was concentrated under reduced pressure to afford crude Intermediate 8, which was used without further purification. m/z (ESI): 260.1 (M+H)⁺.

### Intermediate 9: 2-ethyl-5-(4,4,5,5-tetramethyl-1,3,2-dioxaborolan-2-yl)-2H-pyrazolo[3,4-b]pyridine

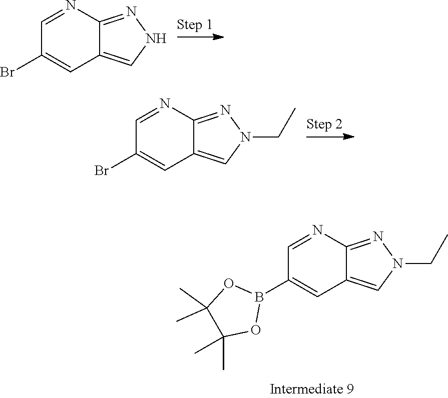 [SMILES: Brc1cnc2n[nH]cc2c1 ; CCn1cc2cc(Br)cnc2n1 ; CCn1cc2cc(B3OC(C)(C)C(C)(C)O3)cnc2n1]

Step 1: A similar protocol as Intermediate 1 was used, with 5-bromo-2H-pyrazolo[3,4-b]pyridine as starting material. m/z (ESI): 226.0 (M+H)⁺.

Step 2: 2-ethyl-5-(4,4,5,5-tetramethyl-1,3,2-dioxaborolan-2-yl)-2H-pyrazolo[3,4-b]pyridine. A similar protocol as Intermediate 1 was used, with 5-bromo-2-ethyl-2H-pyrazolo[3,4-b]pyridine as starting material. m/z (ESI): 274.1 (M+H)⁺.

### Intermediate 10: 3-(4,4,5,5-tetramethyl-1,3,2-dioxaborolan-2-yl)-5,6-dihydro-7H-pyrrolo[3,4-b]pyridin-7-one

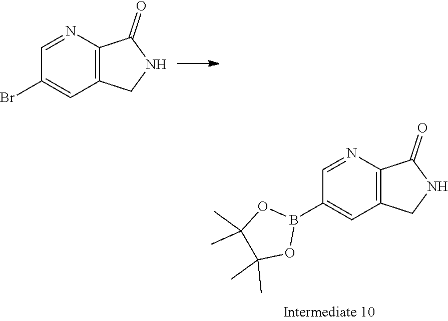 [SMILES: O=C1NCc2cc(Br)cnc21 ; CC1(C)OB(c2cnc3c(c2)CNC3=O)OC1(C)C]

3-(4,4,5,5-tetramethyl-1,3,2-dioxaborolan-2-yl)-5,6-dihydro-7H-pyrrolo[3,4-b]pyridin-7-one. A similar protocol as Intermediate 1, Step 2 was used, with 3-bromo-5,6-dihydro-7H-pyrrolo[3,4-b]pyridin-7-one as starting material. m/z (ESI): 261.1 (M+H)⁺.

### Intermediate 11: 6-(4,4,5,5-tetramethyl-1,3,2-dioxaborolan-2-yl)-[1,2,4]triazolo[4,3-a]pyrimidine

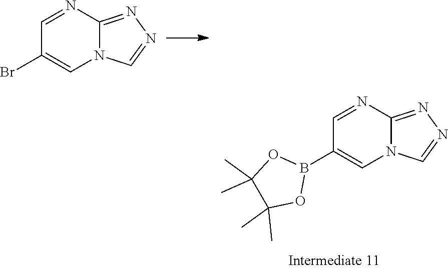 [SMILES: Brc1cnc2nncn2c1 ; CC1(C)OB(c2cnc3nncn3c2)OC1(C)C]

6-(4,4,5,5-tetramethyl-1,3,2-dioxaborolan-2-yl)-[1,2,4]triazolo[4,3-a]pyrimidine. A similar protocol as Intermediate 1, Step 2 was used, with 6-bromo-[1,2,4]triazolo[4,3-a]pyrimidine as starting material. m/z (ESI): 247.1 (M+H)⁺.

### Intermediate 12: 1-methyl-6-(4,4,5,5-tetramethyl-1,3,2-dioxaborolan-2-yl)-1H-[1,2,3]triazolo[4,5-b]pyridine

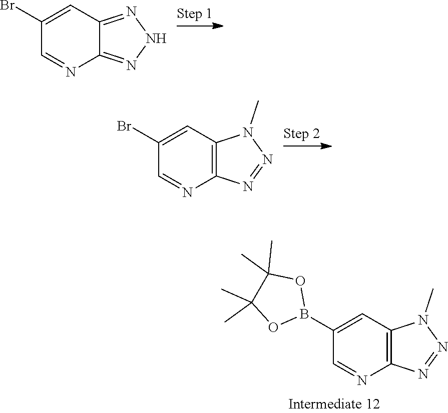 [SMILES: Brc1cnc2n[nH]nc2c1 ; Cn1nnc2ncc(Br)cc21 ; Cn1nnc2ncc(B3OC(C)(C)C(C)(C)O3)cc21]

Step 1: A similar protocol as Intermediate 1 was used, with 6-bromo-2H-[1,2,3]triazolo[4,5-b]pyridine as starting material. m/z (ESI): 213.0 (M+H)⁺.

Step 2: 1-methyl-6-(4,4,5,5-tetramethyl-1,3,2-dioxaborolan-2-yl)-1H-[1,2,3]triazolo[4,5-b]pyridine. A similar protocol as Intermediate 1 was used, with 6-bromo-1-methyl-1H-[1,2,3]triazolo[4,5-b]pyridine as starting material. m/z (ESI): 261.0 (M+H)⁺.

### Intermediate 13: 1-methyl-6-(4,4,5,5-tetramethyl-1,3,2-dioxaborolan-2-yl)-1H-[1,2,3]triazolo[4,5-b]pyridine

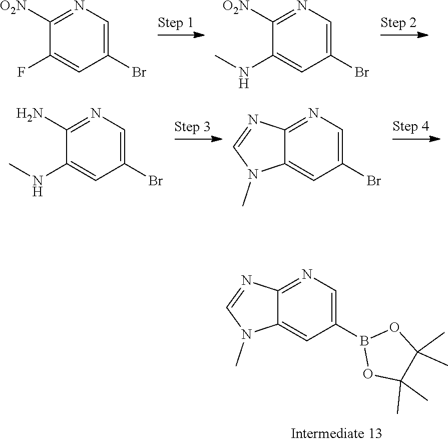

Step 1: 5-bromo-N-methyl-2-nitropyridin-3-amine. To a stirred mixture of 5-bromo-3-fluoro-2-nitropyridine (4.00 g, 18.10 mmol) in THF (56 mL) at rt was added a solution of methylamine in THF (2.0 M, 27.2 mL, 54.3 mmol). The reaction mixture was heated to 50° C. for 2 h. The reaction mass was concentrated, quenched with ice water (500 mL) and stirred for 15 minutes. The resulting precipitate was filtered and dried to give 5-bromo-N-methyl-2-nitropyridin-3-amine (4.00 g, 17.2 mmol, 95% yield). m/z (ESI): 232.0 (M+H)⁺. ¹H NMR (400 MHz, DMSO-d₆) δ 8.08-7.98 (m, 1H), 7.86 (d, J=1.9 Hz, 1H), 7.80 (d, J=1.9 Hz, 1H), 2.96 (d, J=5.0 Hz, 3H).

Step 2: 5-bromo-N³-methylpyridine-2,3-diamine. A stirred mixture of 5-bromo-N-methyl-2-nitropyridin-3-amine (4.00 g, 17.24 mmol) and stannous chloride dihydrate (11.67 g, 51.7 mmol) in THF (80 mL) was heated to 70° C. for 5 h. The reaction mixture was concentrated and taken up in DCM (100 mL) and 10% aq. NaOH (100 mL). The biphasic mixture was stirred until all of the solids dissolved. The organic layer was separated, and aqueous layer was extracted with DCM (2×100 mL). The combined organic layers were washed with brine (50 mL), dried over Na₂SO₄, filtered and concentrated. The crude material was purified by column chromatography, eluting with a gradient of 40% ethyl acetate in pet. ether, to provide 5-bromo-N³-methylpyridine-2,3-diamine (2.9 g, 14.35 mmol, 83% yield). m/z (ESI): 202.0 (M+H)⁺. ¹H NMR (400 MHz, DMSO-d₆) δ 7.29 (d, J=2.1 Hz, 1H), 6.57 (d, J=2.1 Hz, 1H), 5.65 (s, 2H), 5.27-5.20 (m, 1H), 2.70 (d, J=4.9 Hz, 3H).

Step 3: 6-bromo-1-methyl-1H-imidazo[4,5-b]pyridine. A stirred mixture of 5-bromo-N³-methylpyridine-2,3-diamine (1 g, 4.95 mmol) in formic acid (15 mL, 391 mmol) was heated to 100° C. for 18 h. The reaction mass was concentrated under reduced pressure. The resulting residue was quenched with ice water (50 mL) and extracted with DCM (2×100 mL). The combined organic layers were washed with brine (50 mL), dried over Na₂SO₄, filtered and concentrated to afford 6-bromo-1-methyl-1H-imidazo[4,5-b]pyridine (0.7 g, 3.30 mmol, 67% yield). m/z (ESI): 212.0 (M+H)⁺. ¹H NMR (400 MHz, DMSO-d₆) δ 8.50-8.46 (m, 2H), 8.42 (d, J=2.2 Hz, 1H), 3.87 (s, 3H).

Step 4: 1-methyl-6-(4,4,5,5-tetramethyl-1,3,2-dioxaborolan-2-yl)-1H-imidazo[4,5-b]pyridine. A similar protocol as Intermediate 1, Step 2 was used, with 6-bromo-1-methyl-1H-imidazo[4,5-b]pyridine as starting material. m/z (ESI): 260.1 (M+H)⁺.

### Intermediate 14: 3-(4,4,5,5-tetramethyl-1,3,2-dioxaborolan-2-yl)-1,8-naphthyridine

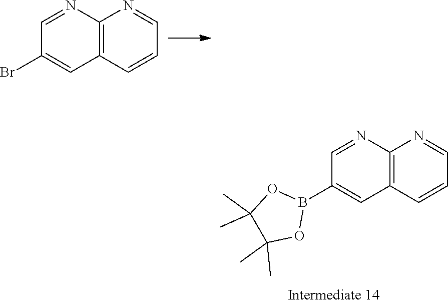 [SMILES: Brc1cnc2ncccc2c1 ; CC1(C)OB(c2cnc3ncccc3c2)OC1(C)C]

3-(4,4,5,5-tetramethyl-1,3,2-dioxaborolan-2-yl)-1,8-naphthyridine. A similar protocol as Intermediate 1, Step 2 was used, with 3-bromo-1,8-naphthyridine as starting material. m/z (ESI): 257.0 (M+H)⁺.

### Intermediate 15: 6-(4,4,5,5-tetramethyl-1,3,2-dioxaborolan-2-yl)-[1,2,4]triazolo[1,5-a]pyrimidine

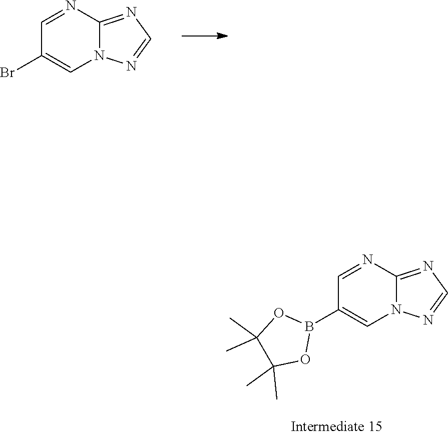 [SMILES: Brc1cnc2ncnn2c1 ; CC1(C)OB(c2cnc3ncnn3c2)OC1(C)C]

6-(4,4,5,5-tetramethyl-1,3,2-dioxaborolan-2-yl)-[1,2,4]triazolo[1,5-a]pyrimidine. A similar protocol as Intermediate 1, Step 2 was used, with 6-bromo-[1,2,4]triazolo[1,5-a]pyrimidine as starting material. m/z (ESI): 247.0 (M+H)⁺.

### Section 2-General Procedures

### Group 1

### Example 1-1: cyclobutyl(6-(2-methyl-2H-pyrazolo[3,4-b]pyridin-5-yl)thieno[2,3-b]pyridin-2-yl)methanol B18

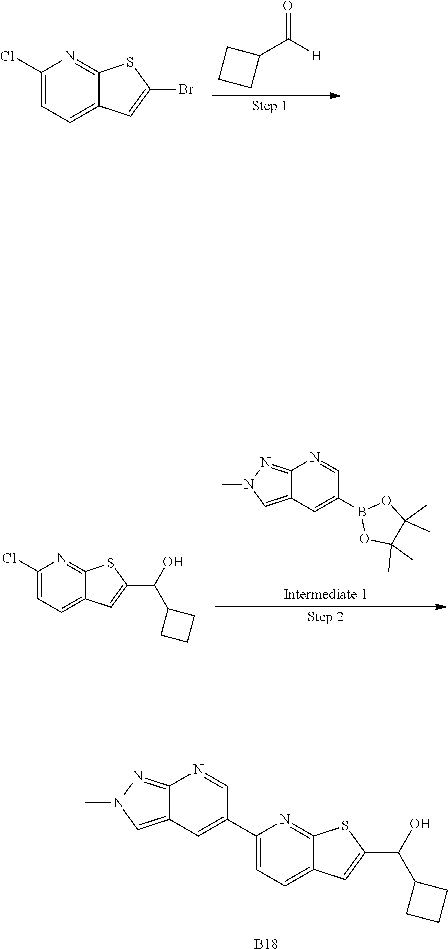

Step 1: (6-chlorothieno[2,3-b]pyridin-2-yl)(cyclobutyl)methanol. To a solution of 2-bromo-6-chlorothieno[2,3-b]pyridine (400 mg, 1.61 mmol) in THF (1 mL) was added n-butyllithium (2.5 M in hexanes, 644 μL, 1.61 mmol) at −78° C. The reaction mixture was stirred for 2 min and cyclobutanecarbaldehyde (135 mg, 1.61 mmol) in THF (1 mL) was added. The reaction mixture was stirred for additional 1 h at −78° C. The reaction mixture was quenched by addition of NH₄Cl (50 mL), extracted with EtOAc (3×50 mL), washed with brine (50 mL), dried over Na₂SO₄, and filtered. The filtrate was concentrated in vacuo. The crude material was absorbed onto a plug of silica gel and purified by column chromatography, eluting with a gradient of 0% to 14% EtOAc in pet. ether, to provide (6-chlorothieno[2,3-b]pyridin-2-yl)(cyclobutyl)methanol (308 mg, 1.21 mmol, 75% yield). m/z (ESI): 254.0 (M+H)⁺.

Step 2: cyclobutyl(6-(2-methyl-2H-pyrazolo[3,4-b]pyridin-5-yl)thieno[2,3-b]pyridin-2-yl)methanol. To a solution of (6-chlorothieno[2,3-b]pyridin-2-yl)(cyclobutyl)methanol (295 mg, 1.16 mmol) in 1,4-dioxane (3 mL) and H₂O (0.6 mL) was added Intermediate 1 (602 mg, 2.33 mmol), K₂CO₃ (482 mg, 3.49 mmol) and Pd(dppf)Cl₂ (47.5 mg, 0.058 mmol). The solution was stirred at 110° C. for 12 h. Upon completion, the reaction mixture was concentrated in vacuo. The residue was purified by prep-HPLC, eluting with a gradient of 25% to 45% MeCN in H₂O (10 mM NH₄HCO₃), to provide Compound B18. m/z (ESI): 351.0 (M+H)⁺. ¹H NMR (400 MHz, DMSO-d₆) δ 9.37 (d, J 2.25 Hz, 1H), 8.90 (d, J 2.38 Hz, 1H), 8.54 (s, 1H), 8.24 (d, J 8.38 Hz, 1H), 8.07 (d, J 8.38 Hz, 1H), 7.25 (s, 1H), 5.87 (br d, J 4.00 Hz, 1H), 4.79 (br d, J 5.13 Hz, 1H), 4.24 (s, 3H), 2.58-2.69 (m, 1H), 1.96-2.05 (m, 2H), 1.86-1.95 (m, 2H), 1.74-1.86 (m, 2H).

Example 1-2: (R)-(6-(2-methyl-2H-pyrazolo[3,4-b]pyridin-5-yl)thieno[2,3-b]pyridin-2-yl)(tetrahydro-2H-pyran-4-yl)methanol B15R; (S)-(6-(2-methyl-2H-pyrazolo[3,4-b]pyridin-5-yl)thieno[2,3-b]pyridin-2-yl)(tetrahydro-2H-pyran-4-yl)methanol B15S

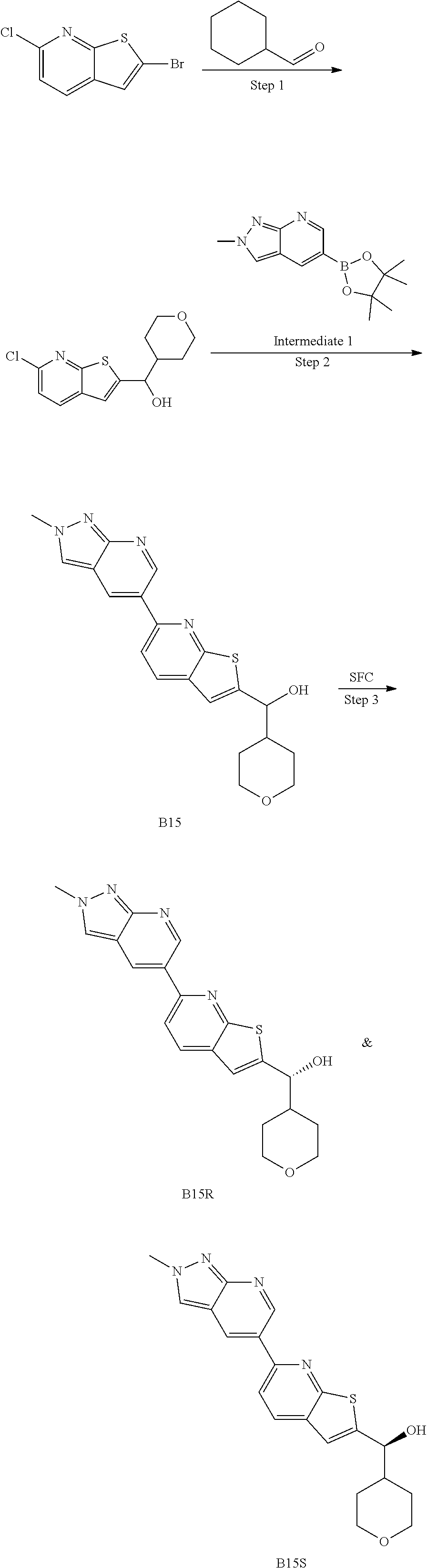

Step 1: (6-chlorothieno[2,3-b]pyridin-2-yl)(tetrahydro-2H-pyran-4-yl)methanol. To a stirred mixture of 2-bromo-6-chlorothieno[2,3-b]pyridine (100 mg, 0.40 mmol) in THF (1 mL) was added n-butyllithium (2.5 M in hexanes, 161 μL, 0.40 mmol) at −70° C. The mixture was stirred at −70° C. for 2 min. Tetrahydro-2H-pyran-4-carbaldehyde (45.9 mg, 0.40 mmol) in THF (1 mL) was added into the mixture. The mixture was stirred at −70° C. for 45 min. The reaction mixture was quenched by addition of sat. aq NH₄Cl (20 mL) at 25° C. The aqueous phase was extracted with EtOAc (4×20 mL). The combined organic layers were washed with brine (20 mL), dried over Na₂SO₄, filtered and concentrated under reduced pressure to give (6-chlorothieno[2,3-b]pyridin-2-yl)(tetrahydro-2H-pyran-4-yl)methanol (80 mg, 0.28 mmol, 70% yield). ¹H NMR (400 MHz, DMSO-d₆) δ 8.21 (d, J 8.25 Hz, 1H), 7.48 (d, J 8.38 Hz, 1H), 7.28 (s, 1H), 6.00 (d, J 4.63 Hz, 1H), 4.65 (dd, J 6.19, 4.94 Hz, 1H), 3.92-3.76 (m, 2H), 3.23 (qd, J 11.72, 2.25 Hz, 2H), 1.86-1.75 (m, 1H), 1.71 (br d, J 13.13 Hz, 1H), 1.41-1.30 (m, 2H), 1.30-1.25 (m, 1H).

Step 2: (6-(2-methyl-2H-pyrazolo[3,4-b]pyridin-5-yl)thieno[2,3-b]pyridin-2-yl)(tetrahydro-2H-pyran-4-yl)methanol. A mixture of (6-chlorothieno[2,3-b]pyridin-2-yl)(tetrahydro-2H-pyran-4-yl)methanol (6.53 g, 23.0 mmol), 2-methyl-5-(4,4,5,5-tetramethyl-1,3,2-dioxaborolan-2-yl)-2H-pyrazolo[3,4-b]pyridine (11.93 g, 46.0 mmol), K₂CO₃ (9.54 g, 69.0 mmol), and Pd(dppf)Cl₂ (1.684 g, 2.30 mmol) in H₂O (13 mL) and 1,4-dioxane (104 mL) was purged with N₂. The mixture was stirred at 110° C. for 12 h under N₂. The reaction mixture was quenched with H₂O (150 mL) and then extracted with EtOAc (5×100 mL). The combined organic layers were washed with brine (500 mL), dried over Na₂SO₄, filtered and concentrated under reduced pressure to give a residue. The residue was purified by chromatography, eluting with a gradient of 0-10% MeOH/EtOAc, to provide Compound B15. m/z (ESI): 381.1 (M+H)⁺. ¹H NMR (400 MHz, DMSO-d₆) δ 9.37 (d, J=2.3 Hz, 1H), 8.90 (d, J=2.3 Hz, 1H), 8.54 (s, 1H), 8.25 (d, J=8.5 Hz, 1H), 8.08 (d, J=8.5 Hz, 1H), 7.27 (s, 1H), 5.96 (d, J=4.5 Hz, 1H), 4.66 (dd, J=5.0, 6.2 Hz, 1H), 4.24 (s, 3H), 3.91-3.79 (m, 2H), 3.30-3.20 (m, 2H), 1.87-1.73 (m, 2H), 1.42-1.28 (m, 3H).

Step 3: SFC Purification. The sample was purified via SFC using a Chiralpak AD-3, 50×4.6 mm I.D., 3 μm, column with a mobile phase of 50% EtOH with 0.1% IPAm using a flowrate of 4 mL/min to generate peak 1 and peak 2:

Peak 1: B15R. Compound B15R was obtained (3.5 g, 9.1 mmol, 39% yield). m/z (ESI): 381.1 (M+H)⁺. ¹H NMR (400 MHz, DMSO-d₆) δ 9.37 (d, J 2.3 Hz, 1H), 8.90 (d, J 2.3 Hz, 1H), 8.54 (s, 1H), 8.25 (d, J 8.5 Hz, 1H), 8.08 (d, J 8.5 Hz, 1H), 7.27 (s, 1H), 5.96 (d, J 4.5 Hz, 1H), 4.66 (dd, J 5.0, 6.2 Hz, 1H), 4.24 (s, 3H), 3.91-3.79 (m, 2H), 3.30-3.20 (m, 2H), 1.87-1.73 (m, 2H), 1.42-1.28 (m, 3H).

Peak 2: B15S. Compound B15S was obtained (3.3 g, 8.6 mmol, 37% yield). m/z (ESI): 381.1 (M+H)⁺. ¹H NMR (400 MHz, DMSO-d₆) δ 9.37 (d, J 2.3 Hz, 1H), 8.90 (d, J 2.3 Hz, 1H), 8.54 (s, 1H), 8.25 (d, J 8.5 Hz, 1H), 8.08 (d, J 8.5 Hz, 1H), 7.27 (s, 1H), 5.96 (d, J 4.5 Hz, 1H), 4.66 (dd, J 5.0, 6.2 Hz, 1H), 4.24 (s, 3H), 3.91-3.79 (m, 2H), 3.30-3.20 (m, 2H), 1.87-1.73 (m, 2H), 1.42-1.28 (m, 3H).

Example 1-3: (R)-(3,3-difluorocyclobutyl)(6-(2-methyl-2H-pyrazolo[3,4-b]pyridin-5-yl)thieno[2,3-b]pyridin-2-yl)methanol B30R; (S)-(3,3-difluorocyclobutyl)(6-(2-methyl-2H-pyrazolo[3,4-b]pyridin-5-yl)thieno[2,3-b]pyridin-2-yl)methanol B30S

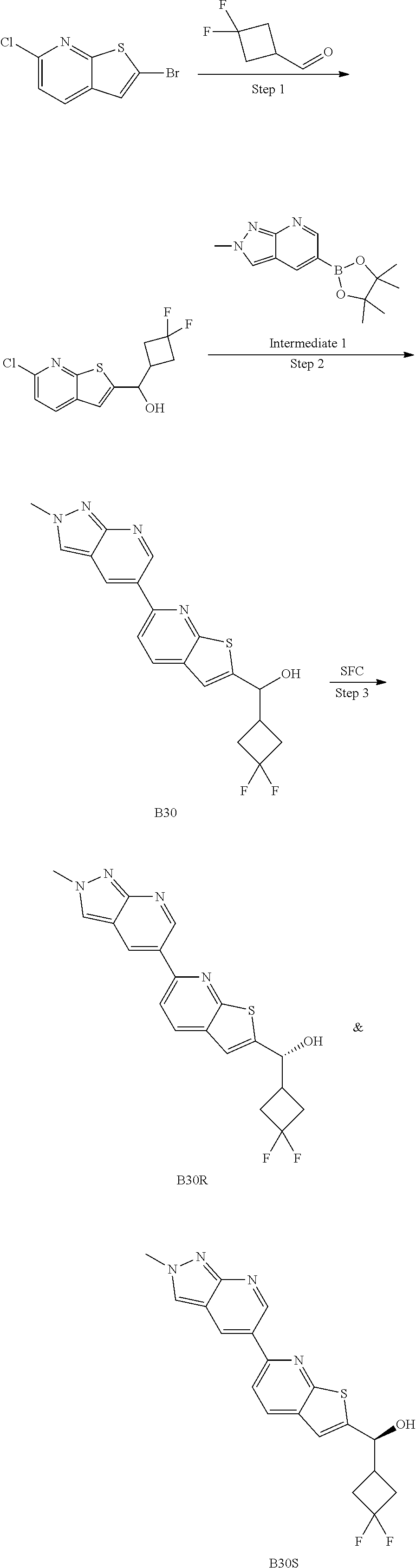

Step 1: (6-chlorothieno[2,3-b]pyridin-2-yl)(3,3-difluorocyclobutyl)methanol. 2—Bromo-6-chlorothieno[2,3-b]pyridine (10.0 g, 40.2 mmol) was dissolved in THF (100 mL). The solution was purged with N₂ and cooled to −70° C. Then, n-butyllithium (2.5 M in hexanes, 17.70 mL, 44.3 mmol) was added dropwise, and the mixture was stirred at −70° C. for 10 min. 3,3-Difluorocyclobutane-1-carbaldehyde (5.80 g, 48.3 mmol) was added, and the mixture was stirred at −70° C. for 1 h. The reaction mixture was quenched by addition of cold sat. aq. NH₄Cl (100 mL) and extracted with EtOAc (3×100 mL). The combined organic phases were washed with brine, dried over Na₂SO₄, filtered and concentrated under reduced pressure to give a crude product. The residue was purified by chromatography, eluting with 0-25% EtOAc/pet. ether, to provide (6-chlorothieno[2,3-b]pyridin-2-yl)(3,3-difluorocyclobutyl)methanol (9.00 g, 31.1 mmol, 77% yield). ¹H NMR (400 MHz, DMSO-d₆) δ 8.22 (d, J 8.4 Hz, 1H), 7.49 (d, J 8.4 Hz, 1H), 7.34 (d, J 0.8 Hz, 1H), 6.31 (d, J 5.1 Hz, 1H), 4.91 (t, J 4.9 Hz, 1H), 2.63-2.51 (m, 5H).

Step 2: (3,3-difluorocyclobutyl)(6-(2-methyl-2H-pyrazolo[3,4-b]pyridine-5-yl)thieno[2,3-b]pyridine-2-yl)methanol. To a mixture of (6-chlorothieno[2,3-b]pyridine-2-yl)(3,3-difluorocyclobutyl)methanol (7.50 g, 25.9 mmol), 2-methyl-5-(4,4,5,5-tetramethyl-1,3,2-dioxaborolan-2-yl)-2H-pyrazolo[3,4-b]pyridine (8.72 g, 33.7 mmol), and K₂CO₃ (10.73 g, 78 mmol) in 1,4-dioxane (150 mL) and H₂O (15 mL) was added Pd(dppf)Cl₂ (1.89 g, 2.59 mmol) at 20° C. The mixture was stirred at 110° C. for 12 h under N₂. The reaction mixture was quenched by addition of H₂O (500 mL) and extracted with EtOAc (8×200 mL). The combined organic phases were washed with brine, dried over Na₂SO₄, filtered and concentrated under reduced pressure to give the crude product. The residue was purified by chromatography, eluting with a gradient of 0-20% MeOH/EtOAc, to provide Compound B30 (7.20 g, 16.21 mmol, 63% yield). m/z (ESI): 387.3 (M+H)⁺. ¹H NMR (400 MHz, DMSO-d₆) δ 9.37 (d, J 2.0 Hz, 1H), 8.91 (d, J 2.4 Hz, 1H), 8.54 (s, 1H), 8.26 (d, J 8.4 Hz, 1H), 8.09 (d, J 8.4 Hz, 1H), 7.33 (s, 1H), 6.26 (d, J 4.8 Hz, 1H), 4.93 (s, 1H), 4.23 (s, 3H), 2.61-2.48 (m, 5H).

Step 3: SFC Purification. The sample was purified via SFC using a Chiralpak IH-3, 50×4.6 mm I.D., 3 um, column with a mobile phase of 50% EtOH with 0.1% IPAm using a flowrate of 3.4 mL/min to generate peak 1 and peak 2.

Peak 1: B30R. Compound B30R was obtained (2.00 g, 5.12 mmol, 42% yield). m/z (ESI): 387.3 (M+H)⁺. ¹H NMR (400 MHz, DMSO-d₆) δ 9.37 (d, J 2.3 Hz, 1H), 8.91 (d, J 2.3 Hz, 1H), 8.54 (s, 1H), 8.26 (d, J 8.5 Hz, 1H), 8.09 (d, J 8.4 Hz, 1H), 7.33 (s, 1H), 6.26 (d, J 4.6 Hz, 1H), 4.93 (br s, 1H), 4.24 (s, 3H), 2.67-2.53 (m, 5H).

Peak 2: B30S. Compound B30S was obtained (2.00 g, 5.12 mmol, 42% yield). m/z (ESI): 387.3 (M+H)⁺. ¹H NMR (400 MHz, DMSO-d₆) δ 9.37 (d, J 2.3 Hz, 1H), 8.91 (d, J 2.3 Hz, 1H), 8.54 (s, 1H), 8.26 (d, J 8.5 Hz, 1H), 8.09 (d, J 8.4 Hz, 1H), 7.33 (s, 1H), 6.26 (d, J 4.6 Hz, 1H), 4.93 (br s, 1H), 4.24 (s, 3H), 2.67-2.53 (m, 5H).

Example 1-4: (R)-(6-(3-methyl-3H-[1,2,3]triazolo[4,5-b]pyridin-6-yl)thieno[2,3-b]pyridin-2-yl)(tetrahydro-2H-pyran-4-yl)methanol B41R; (S)-(6-(3-methyl-3H-[1,2,3]triazolo[4,5-b]pyridin-6-yl)thieno[2,3-b]pyridin-2-yl)(tetrahydro-2H-pyran-4-yl)methanol B41S

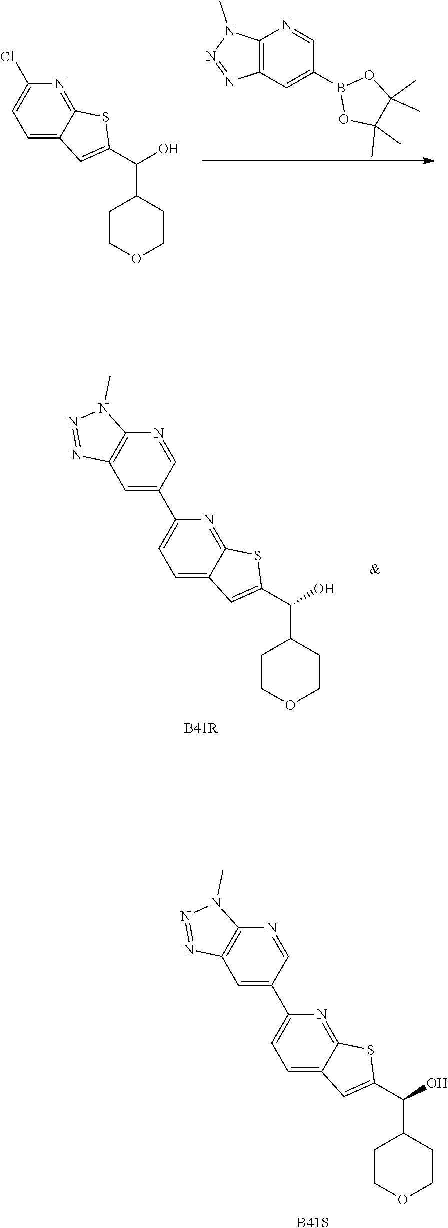 [SMILES: OC(c1cc2ccc(Cl)nc2s1)C1CCOCC1 ; Cn1nnc2cc(B3OC(C)(C)C(C)(C)O3)cnc21 ; "B41R" ; Cn1nnc2cc(-c3ccc4cc([C@H](O)C5CCOCC5)sc4n3)cnc21 ; "B41S" ; Cn1nnc2cc(-c3ccc4cc([C@@H](O)C5CCOCC5)sc4n3)cnc21]

To a solution of (6-chlorothieno[2,3-b]pyridin-2-yl)(tetrahydro-2H-pyran-4-yl)methanol (350 mg, 1.23 mmol) and 3-methyl-6-(4,4,5,5-tetramethyl-1,3,2-dioxaborolan-2-yl)-3H-[1,2,3]triazolo[4,5-b]pyridine (481 mg, 1.85 mmol) in N,N-dimethylacetamide (3 mL) and H₂O (0.6 mL) was added Na₂CO₃ (327 mg, 3.08 mmol) and Pd(PPh₃)₄(71.3 mg, 0.062 mmol). Then, the mixture was stirred at 120° C. for 1 h. The reaction mixture was poured into H₂O (40 mL), extracted with EtOAc (30 mL×3), washed with brine (20 mL), dried over Na₂SO₄, filtered and concentrated under reduced pressure to provide a crude material. The crude material was purified by column chromatography, eluting with 0-55% EtOAc in pet. ether, to obtain a mixture of B41R and B41S (280 mg, 0.73 mmol, 60% yield). m/z (ESI): 382.1 (M+H)⁺.

Step 3: SFC Purification. The sample was purified via SFC using a Chiralpak AD-3, 50×4.6 mm I.D., 3 um, column with a mobile phase of 50% EtOH with 0.1% IPAm using a flowrate of 4 mL/min to generate peak 1 and peak 2.

Peak 1: B41R. Compound B41R was obtained (131 mg, 0.342 mmol, 28% yield). m/z (ESI): 382.0 (M+H)⁺. ¹H NMR (400 MHz, DMSO-d₆) δ 9.57 (d, J 2.00 Hz, 1H), 9.20 (d, J 2.00 Hz, 1H), 8.35-8.29 (m, 1H), 8.22 (d, J 8.38 Hz, 1H), 7.32 (s, 1H), 5.99 (s, 1H), 4.69 (d, J 6.75 Hz, 1H), 4.37 (s, 3H), 3.95-3.78 (m, 2H), 3.31-3.19 (m, 2H), 1.85 (qd, J 7.36, 3.31 Hz, 1H), 1.76 (br d, J 13.13 Hz, 1H), 1.45-1.28 (m, 3H).

Peak 2: B41S. Compound B41S was obtained (97 mg, 0.255 mmol, 21% yield). m/z (ESI): 382.0 (M+H)⁺. ¹H NMR (400 MHz, DMSO-d₆) δ 9.55 (d, J 2.00 Hz, 1H), 9.20 (d, J 2.00 Hz, 1H), 8.32-8.30 (m, 1H), 8.20 (d, J 8.38 Hz, 1H), 7.30 (s, 1H), 5.99 (s, 1H), 4.68 (d, J 6.75 Hz, 1H), 4.36 (s, 3H), 3.90-3.81 (m, 2H), 3.33-3.24 (m, 2H), 1.84 (qd, J 7.36, 3.31 Hz, 1H), 1.74 (br d, J 13.13 Hz, 1H), 1.42-1.32 (m, 3H).

Example 1-5: (R)-(3,3-difluorocyclobutyl)(6-(2-methylimidazo[1,2-a]pyridin-6-yl)thieno[2,3-b]pyridin-2-yl)methanol B48R; (S)-(3,3-difluorocyclobutyl)(6-(2-methylimidazo[1,2-a]pyridin-6-yl)thieno[2,3-b]pyridin-2-yl)methanol B48S

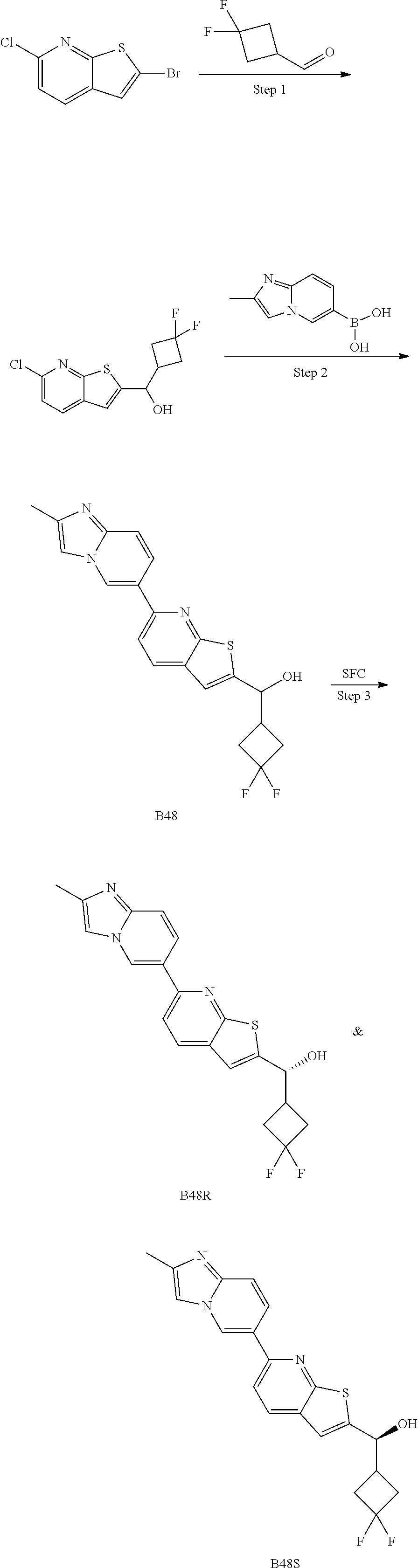

Step 1: (6-chlorothieno[2,3-b]pyridin-2-yl)(3,3-difluorocyclobutyl)methanol. To a solution of 2-bromo-6-chlorothieno[2,3-b]pyridine (5.00 g, 20.1 mmol) in THF (50 mL) was added a solution of n-butyllithium in hexanes (2.5 M, 8.45 mL, 21.1 mmol) at −78° C. The reaction mixture was stirred for 2 min, and 3,3-difluorocyclobutane-1-carbaldehyde (2.66 g, 22.1 mmol) in THF (50 mL) was added. The reaction mixture was stirred for an additional 0.5 h at −78° C. The reaction mixture was diluted with H₂O (100 mL). Then, the mixture was extracted with EtOAc (3×50 mL). The combined organic layers were washed with brine (50 mL), dried over Na₂SO₄, filtered and concentrated under reduced pressure. The residue was purified by chromatography, eluting with a gradient of 0-7% MeOH in (1:1) EtOAc:pet. ether, to provide (6-chlorothieno[2,3-b]pyridin-2-yl)(3,3-difluorocyclobutyl)methanol (6.00 g, 20.7 mmol, 103% crude yield). ¹H NMR (400 MHz, CHLOROFORM-d) 6:7.92 (d, J 8.4 Hz, 1H), 7.31 (d, J 8.3 Hz, 1H), 7.14 (s, 1H), 4.99 (br d, J 6.0 Hz, 1H), 2.74-2.66 (m, 2H), 2.62-2.57 (m, 2H), 2.54-2.44 (m, 1H).

Step 2: (3,3-difluorocyclobutyl)(6-(2-methylimidazo[1,2-a]pyridin-6-yl)thieno[2,3-b]pyridin-2-yl)methanol. A mixture of (2-methylimidazo[1,2-a]pyridin-6-yl)boronic acid (146 mg, 0.83 mmol), (6-chlorothieno[2,3-b]pyridin-2-yl)(3,3-difluorocyclobutyl)methanol (200 mg, 0.70 mmol), K₂CO₃ (239 mg, 1.73 mmol), and Pd(dppf)Cl₂ (50.5 mg, 0.069 mmol) in H₂O (0.5 mL) and 1,4-dioxane (3 mL) was purged with N₂. The mixture was stirred at 110° C. for 12 h under N₂. The reaction mixture was quenched by addition of H₂O (30 mL) and then extracted with EtOAc (5×30 mL). The combined organic layers were washed with brine (50 mL), dried over Na₂SO₄, filtered, and concentrated under reduced pressure to give a residue. The residue was purified by chromatography, eluting with a gradient of 0-10% MeOH/EtOAc to provide Compound B48 (50 mg, 0.10 mmol, 15% yield). m/z (ESI): 386.0 (M+H)⁺. ¹H NMR (400 MHz, DMSO-d₆) δ 9.30 (s, 1H), 8.25 (d, J 8.8 Hz, 1H), 7.99-7.94 (m, 2H), 7.80 (s, 1H), 7.53 (d, J 9.2 Hz, 1H), 7.32 (s, 1H), 6.25 (d, J 5.2 Hz, 1H), 4.92 (d, J 4.4 Hz, 1H), 2.62-2.54 (m, 5H), 2.36 (s, 3H).

Step 3: SFC Purification. The sample was purified via SFC using a Chiralpak IG-3, 50×4.6 mm I.D., 3 um, column with a mobile phase of 50% EtOH with 0.1% IPAm using a flowrate of 3.4 mL/min to generate peak 1 and peak 2:

Peak 1: B48R. Compound B48R was obtained (15.0 mg, 0.039 mmol, 38% yield). m/z (ESI): 386.0 (M+H)⁺. ¹H NMR (400 MHz, DMSO-d₆) δ 9.31 (s, 1H), 8.25 (d, J 8.5 Hz, 1H), 8.00-7.93 (m, 2H), 7.80 (s, 1H), 7.53 (d, J 9.4 Hz, 1H), 7.32 (s, 1H), 6.26 (d, J 5.0 Hz, 1H), 4.92 (t, J 4.4 Hz, 1H), 2.62-2.53 (m, 5H), 2.36 (s, 3H).

Peak 2: B48S. Compound B48S was obtained (11.2 mg, 0.028 mmol, 28% yield). m/z (ESI): 386.1 (M+H)⁺. ¹H NMR (400 MHz, DMSO-d₆) δ 9.30 (d, J 0.6 Hz, 1H), 8.25 (d, J 8.5 Hz, 1H), 8.00 -7.93 (m, 2H), 7.80 (s, 1H), 7.53 (d, J 9.5 Hz, 1H), 7.32 (s, 1H), 6.25 (d, J 5.0 Hz, 1H), 4.94-4.90 (m, 1H), 2.63-2.53 (m, 5H), 2.36 (s, 3H).

Example 1-6: cis-3-(difluoromethyl)-1-(6-(2-methyl-2H-pyrazolo[3,4-b]pyridin-5-yl)thieno[2,3-b]pyridin-2-yl)cyclobutanol C30C

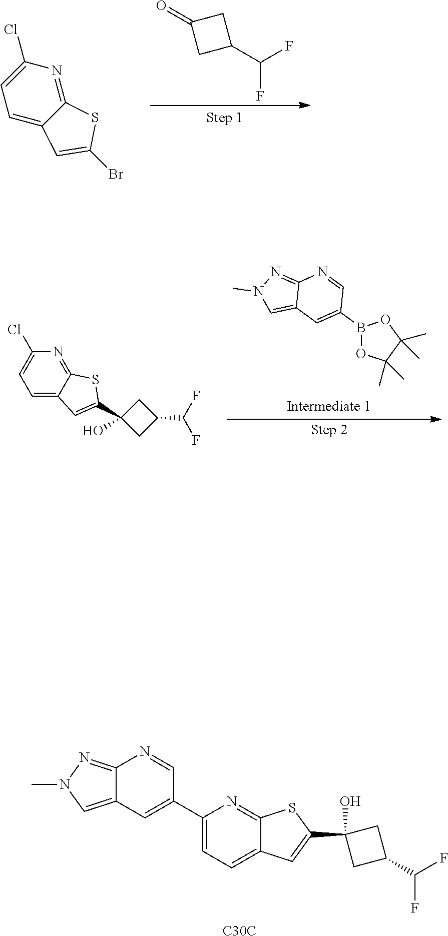

Step 1: cis-1-(6-chlorothieno[2,3-b]pyridin-2-yl)-3-(difluoromethyl)cyclobutan-1-ol. To a solution of 2-bromo-6-chlorothieno[2,3-b]pyridine (0.25 g, 1.01 mmol) and 3-(difluoromethyl)cyclobutan-1-one (0.242 g, 2.01 mmol) in THF (4.00 mL) was added a solution of n-butyllithium in THF (2.5 M, 1.01 mL, 2.51 mmol) in one portion at −78° C. After addition, the resulting mixture was stirred at −78° C. for 1 h. Then, the mixture was quenched by addition of sat. aq. NH₄Cl solution (50 mL), extracted with EtOAc (2×50 mL), washed with brine (50 mL), dried over Na₂SO₄, and filtered. The filtrate was concentrated under reduced pressure to give a residue that was purified by column chromatography, eluting with 20-25% pet. ether/EtOAc, to give cis-1-(6-chlorothieno[2,3-b]pyridin-2-yl)-3-(difluoromethyl)cyclobutan-1-ol. m/z (ESI): 289.9 (M+H)⁺.

Step 2: cis-3-(difluoromethyl)-1-(6-(2-methyl-2H-pyrazolo[3,4-b]pyridin-5-yl)thieno[2,3-b]pyridin-2-yl)cyclobutanol. A mixture of cis-1-(6-chlorothieno[2,3-b]pyridin-2-yl)-3-(difluoromethyl)cyclobutan-1-ol (0.25 g, 0.86 mmol), Intermediate 1 (0.291 g, 1.12 mmol), and K₂CO₃ (0.358 g, 2.59 mmol) in 1,4-dioxane (5.00 mL) and H₂O (0.50 mL) was degassed with N₂ for 10 min. To the degassed mixture was added Pd(dppf)Cl₂ (0.063 g, 0.086 mmol) at rt. The reaction mixture was then stirred at 110° C. for 16 h. The reaction mixture was filtered through celite and concentrated under reduced pressure to give a crude material that was purified by Preparative HPLC, eluting with a gradient of 5-95% (0.1% formic acid) H₂O in ACN over 3.0 min with a flow rate of 2.0 mL/min, to provide Compound C30C (100 mg). m/z (ESI): 387.0 (M+H)⁺. ¹H NMR (400 MHz, DMSO-d₆): δ 9.39 (d, J=2.4 Hz, 1H), 8.92 (d, J=2.3 Hz, 1H), 8.55 (s, 1H), 8.26 (d, J=8.4 Hz, 1H), 8.11 (d, J=8.5 Hz, 1H), 7.50 (s, 1H), 6.49 (s, 1H), 6.17 (dt, J=57.2, 3.4 Hz, 1H), 4.25 (s, 3H), 2.65-2.53 (m, 3H), 2.44 (q, J=8.2, 6.5 Hz, 2H).

Example 1-7: cis-3-methoxy-3-methyl-1-(6-(2-methyl-2H-pyrazolo[3,4-b]pyridin-5-yl)thieno[2,3-b]pyridin-2-yl)cyclobutanol C59C

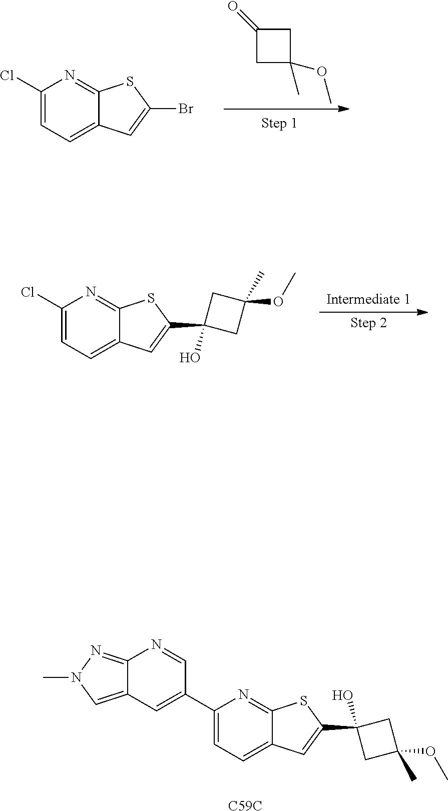

Step 1: 1-(6-chlorothieno[2,3-b]pyridin-2-yl)-3-methoxy-3-methylcyclobutan-1-ol. To a solution of 2-bromo-6-chlorothieno[2,3-b]pyridine (1 g, 4.02 mmol) in toluene (40.0 mL) at −78° C. was added n-butyllithium in hexanes (2.5 M, 2.41 mL, 6.04 mmol). This solution was stirred for 2 min, after which a solution of 3-methoxy-3-methylcyclobutan-1-one (0.689 g, 6.04 mmol) in toluene (10 mL) was added dropwise. The reaction was stirred for 10 min at −78° C. Then, the reaction was diluted with satd. aq. NH₄Cl (50 mL) and extracted with EtOAc (2×50 mL). The combined organic extracts were washed with H₂O (50 mL), dried over Na₂SO₄, filtered, and concentrated. The crude material was purified by chromatography, eluting with a gradient of 15%-30% EtOAc in hexane, to provide cis-1-(6-chlorothieno[2,3-b]pyridin-2-yl)-3-methoxy-3-methylcyclobutan-1-ol (500 mg, 1.76 mmol, 44% yield); m/z (ESI): 284.1 (M+H); ¹H NMR (400 MHz, DMSO-d₆) δ 8.23 (d, J=8.4 Hz, 1H), 7.50 (d, J=8.4 Hz, 1H), 7.42 (s, 1H), 6.29 (s, 1H), 3.12 (s, 3H), 2.65-2.45 (m, 4H), 1.23 (s, 3H); and trans-1-(6-chlorothieno[2,3-b]pyridin-2-yl)-3-methoxy-3-methylcyclobutan-1-ol (190 mg, 0.67 mmol, 17% yield); m/z (ESI): 284.0 (M+H); ¹H NMR (400 MHz, DMSO-d₆) δ 8.23 (d, J=8.4 Hz, 1H), 7.52-7.43 (m, 1H), 7.34 (s, 1H), 6.33 (s, 1H), 3.11 (s, 3H), 2.63-2.54 (m, 2H), 2.36-2.26 (m, 2H), 1.47 (s, 3H).

Step 2: cis-3-methoxy-3-methyl-1-(6-(2-methyl-2H-pyrazolo[3,4-b]pyridin-5-yl)thieno[2,3-b]pyridin-2-yl)cyclobutanol. A solution of cis-1-(6-chlorothieno[2,3-b]pyridin-2-yl)-3-methoxy-3-methylcyclobutan-1-ol (0.19 g, 0.67 mmol), 2-methyl-5-(4,4,5,5-tetramethyl-1,3,2-dioxaborolan-2-yl)-2H-pyrazolo[3,4-b]pyridine (0.26 g, 1.0 mmol) and K₂CO₃ (0.278 g, 2.0 mmol) in 1,4-dioxane (6.84 mL) and H₂O (0.76 mL) was purged with N₂ gas for 5 min. After purging, SPhos Pd G3 (52 mg, 0.067 mmol) was added. The reaction mixture was stirred at 110° C. for 2 h. Then, the reaction mass was diluted with EtOAc (30 mL) and filtered through celite. The filtrate was washed with satd. NH₄Cl (30 mL), dried over Na₂SO₄,filtered and concentrated. The residue was purified by reverse-phase preparative HPLC, eluting with a linear gradient of 5%-95% ACN in (0.1% NH₃) H₂O using a flow rate 15 mL/min, to provide Compound C59C. m/z (ESI): 381.2 (M+H)⁺. ¹H NMR (400 MHz,) 69.38 (d, J=2.3 Hz, 1H), 8.91 (d, J=2.3 Hz, 1H), 8.55 (s, 1H), 8.27 (d, J=8.4 Hz, 1H), 8.09 (d, J=8.4 Hz, 1H), 7.32 (s, 1H), 6.26 (s, 1H), 4.24 (s, 3H), 3.13 (s, 3H), 2.62 (d, J=13.2 Hz, 2H), 2.35 (d, J=13.4 Hz, 2H), 1.50 (s, 3H).

Example 1-8: trans-3-hydroxy-1-methyl-3-(6-(2-methyl-2H-pyrazolo[3,4-b]pyridin-5-yl)thieno[2,3-b]pyridin-2-yl)cyclobutanecarbonitrile C63T; cis-3-hydroxy-1-methyl-3-(6-(2-methyl-2H-pyrazolo[3,4-b]pyridin-5-yl)thieno[2,3-b]pyridin-2-yl)cyclobutanecarbonitrile C63C

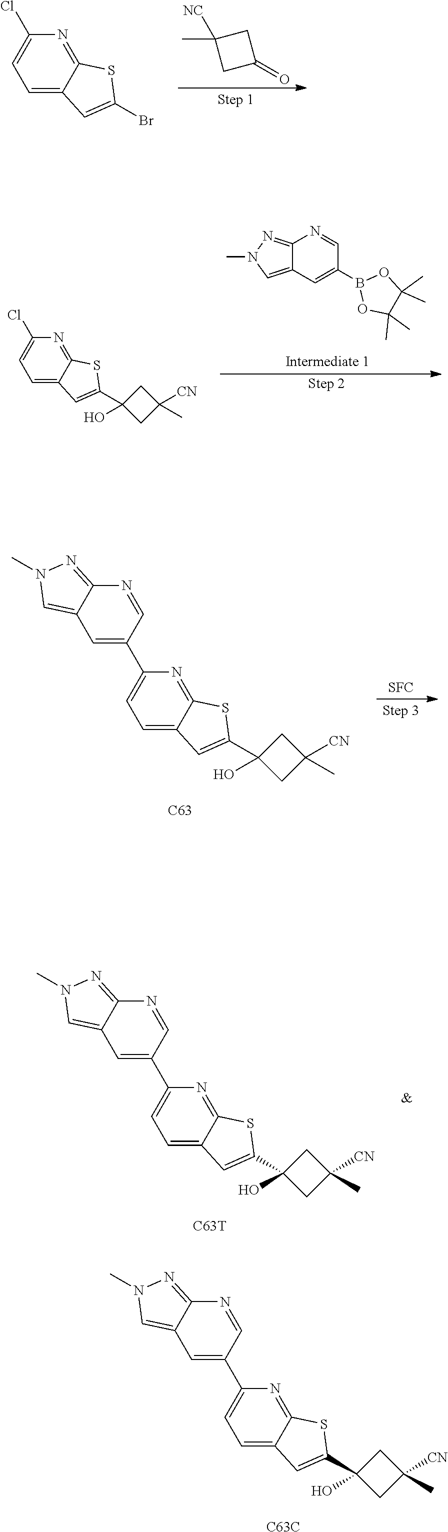

Step 1: 3-(6-chlorothieno[2,3-b]pyridin-2-yl)-3-hydroxy-1-methylcyclobutane-1-carbonitrile. To a solution of 2-bromo-6-chlorothieno[2,3-b]pyridine (1 g, 4.02 mmol) in THF (25.00 mL) was added a solution of n-butyllithium in hexanes (2.5 M, 2.414 mL, 6.04 mmol) dropwise at −78° C., and the mixture was stirred at −78° C. for 1 min. Then, 1-methyl-3-oxocyclobutane-1-carbonitrile (0.659 g, 6.04 mmol) was dissolved in THF (2 mL) and added dropwise, and the reaction was stirred at −78° C. for 5 min. Then, the reaction was quenched by addition of satd. aq. NH₄Cl (50 mL), extracted with EtOAc (30 mL×3), washed with brine (30 mL), dried over Na₂SO₄, and concentrated under reduced pressure to give a crude material. The crude material was purified by column chromatography, eluting with 15% EtOAc in pet. ether, to obtain 3-(6-chlorothieno[2,3-b]pyridin-2-yl)-3-hydroxy-1-methylcyclobutane-1-carbonitrile (0.5 g, 1.80 mmol, 44% yield). m/z (ESI): 279.2 (M+H)⁺. ¹H NMR (400 MHz, DMSO-d₆) δ 8.23 (d, J=8.4 Hz, 1H), 7.51 (d, J=8.4 Hz, 1H), 7.48 (s, 1H), 6.74 (s, 1H), 2.91-2.79 (m, 2H), 2.78-2.68 (m, 2H), 1.53 (s, 3H).

Step 2: 3-hydroxy-1-methyl-3-(6-(2-methyl-2H-pyrazolo[3,4-b]pyridin-5-yl)thieno[2,3-b]pyridin-2-yl)cyclobutane-1-carbonitrile. A mixture of 3-(6-chlorothieno[2,3-b]pyridin-2-yl)-3-hydroxy-1-methylcyclobutane-1-carbonitrile (0.5 g, 1.80 mmol), Intermediate 1 (0.60 g, 2.33 mmol) and K₂CO₃ (0.74 g, 5.38 mmol) in 1,4-dioxane (18 mL) and H₂O (2 mL) was purged with N₂ gas for 5 min. After purging, SPhos Pd G3 (0.14 g, 0.18 mmol) was added, and the reaction mixture was stirred at 110° C. for 3 h. After, the reaction mass was diluted with EtOAc, filtered, and washed with EtOAc (50 mL). The filtrate was concentrated under reduced pressure to afford a crude material. The crude material was purified by preparative HPLC, eluting with a gradient of 5-95% (0.1% NH₃) H₂O in ACN with a flow rate of 15 mL/min, to provide a mixture of Compound C63T and Compound C63C (0.5 g, 1.33 mmol, 74% yield). m/z (ESI): 376.2 (M+H)⁺.

Step 3: SFC Purification. The sample was purified via SFC using a Chiralel OJ-H 250×30 mm, 5 μm, column with a mobile phase of 40% MeOH/CO₂ using a flowrate of 130 mL/min to generate peak 1 and peak 2.

Peak 1: C63T. Compound C63T was obtained (145 mg, 386 mmol, 26% yield). m/z (ESI): 376.2 (M+H)⁺. ¹H NMR (400 MHz, DMSO-d₆) δ 9.39 (d, J=2.3 Hz, 1H), 8.93 (d, J=2.3 Hz, 1H), 8.56 (s, 1H), 8.32 (d, J=8.4 Hz, 1H), 8.12 (d, J=8.4 Hz, 1H), 7.53 (s, 1H), 6.65 (s, 1H), 4.25 (s, 3H), 3.18-3.04 (m, 2H), 2.58 (d, J=13.3 Hz, 2H), 1.68 (s, 3H).

Peak 2: C63C. Compound C63C was obtained (200 mg, 533 mmol, 36% yield). m/z (ESI): 376.2 (M+H)⁺. ¹H NMR (400 MHz, DMSO-d₆) δ 9.38 (d, J=2.3 Hz, 1H), 8.93 (d, J=2.4 Hz, 1H), 8.55 (s, 1H), 8.28 (d, J=8.4 Hz, 1H), 8.12 (d, J=8.4 Hz, 1H), 7.46 (s, 1H), 6.70 (s, 1H), 4.24 (s, 3H), 2.92-2.82 (m, 2H), 2.81-2.69 (m, 2H), 1.55 (s, 3H).

Example 1-9: cis-1-(2-(2-methyl-2H-pyrazolo[3,4-b]pyridin-5-yl)thieno[2,3-d]pyrimidin-6-yl)-3-(trifluoromethyl)cyclobutanol C71C

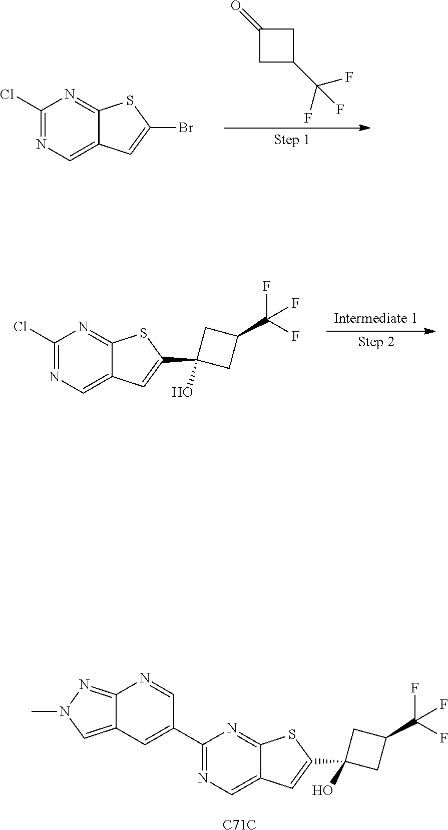 [SMILES: Clc1ncc2cc(Br)sc2n1 ; O=C1CC(C(F)(F)F)C1 ; O[C@]1(c2cc3cnc(Cl)nc3s2)C[C@@H](C(F)(F)F)C1 ; Cn1cc2cc(-c3ncc4cc([C@]5(O)C[C@@H](C(F)(F)F)C5)sc4n3)cnc2n1 ; "C71C"]

Step 1: cis-1-(2-chlorothieno[2,3-d]pyrimidin-6-yl)-3-(trifluoromethyl)cyclobutan-1-ol. To a solution of 6-bromo-2-chlorothieno[2,3-d]pyrimidine (200 mg, 0.8 mmol) in toluene (6 mL) was added n-butyllithium in THF (2.5 M, 0.417 mL, 1.04 mmol) dropwise at −78° C. After stirring for 1 min, 3-(trifluoromethyl)cyclobutan-1-one (221 mg, 1.6 mmol) in toluene (1 mL) was added, and the mixture was stirred at −78° C. for 1 h. Then, the reaction was quenched by addition of satd. aq. NH₄Cl (50 mL) and extracted with EtOAc (2×50 mL). The combined organic layers were washed with brine (50 mL), dried over Na₂SO₄, filtered, and concentrated under reduced pressure. The residue was purified by column chromatography, eluting with a gradient of 5%-95% EtOAc in pet. ether, to give cis-1-(2-chlorothieno[2,3-d]pyrimidin-6-yl)-3-(trifluoromethyl)cyclobutan-1-ol (120 mg, 0.39 mmol, 49% yield). m/z (ESI): 309.0 (M+H)⁺. ¹H NMR (400 MHz, DMSO-d₆) δ 9.15 (s, 1H), 7.72 (s, 1H), 6.92 (s, 1H), 3.13 (h, J=9.0 Hz, 1H), 2.78-2.68 (m, 2H), 2.61-2.51 (m, 2H).

Step 2: cis-1-(2-(2-methyl-2H-pyrazolo[3,4-b]pyridin-5-yl)thieno[2,3-d]pyrimidin-6-yl)-3-(trifluoromethyl)cyclobutanol. To a solution of cis-1-(2-chlorothieno[2,3-d]pyrimidin-6-yl)-3-(trifluoromethyl)cyclobutan-1-ol (0.15 g, 0.49 mmol) in 1,4-dioxane (10 mL) and water (2 mL) was added 2-methyl-5-(4,4,5,5-tetramethyl-1,3,2-dioxaborolan-2-yl)-2H-pyrazolo[3,4-b]pyridine (164 mg, 0.63 mmol) and K₂CO₃ (201 mg, 1.46 mmol). The solution was degassed with N₂ gas for 5 minutes, and SPhos Pd G3 (38 mg, 0.049 mmol) was added. The reaction mixture was stirred at 90° C. for 2 h. The reaction mixture was diluted with H₂O (10 mL) and extracted with EtOAc (2×50 mL). The combined organic extracts were washed with satd. aq. NaCl (50 mL) and dried over Na₂SO₄. The mixture was filtered and concentrated. The crude material was purified by reverse-phase preparative HPLC, eluting with a gradient of 5%-95% ACN in (0.1% NH₃) H₂O and a flow rate of 15 mL/min, to provide Compound C71C (0.12 g, 0.28 mmol, 58% yield). m/z (ESI): 406.2 (M+H)⁺. ¹H NMR (400 MHz, DMSO-d₆) δ 9.64 (d, J=2.1 Hz, 1H), 9.30 (s, 1H), 9.24 (d, J=2.2 Hz, 1H), 8.60 (s, 1H), 7.70 (s, 1H), 6.85 (s, 1H), 4.25 (s, 3H), 3.13 (h, J=9.0 Hz, 1H), 2.83-2.72 (m, 2H), 2.62-2.52 (m, 2H).

Example 1-10: cis-1-(5-(2-methyl-2H-pyrazolo[3,4-b]pyridin-5-yl)[1,3]thiazolo[5,4-b]pyridin-2-yl)-3-(trifluoromethyl)cyclobutanol C74C

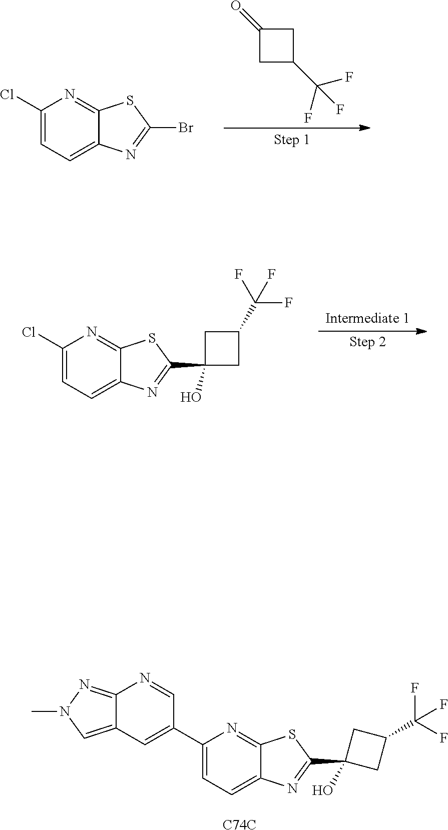 [SMILES: Clc1ccc2nc(Br)sc2n1 ; O=C1CC(C(F)(F)F)C1 ; O[C@]1(c2nc3ccc(Cl)nc3s2)C[C@H](C(F)(F)F)C1 ; Cn1cc2cc(-c3ccc4nc([C@]5(O)C[C@@H](C(F)(F)F)C5)sc4n3)cnc2n1 ; "C74C"]

Step 1:cis-1-(5-chlorothiazolo[5,4-b]pyridin-2-yl)-3-(trifluoromethyl)cyclobutan-1-ol. To a solution of 2-bromo-5-chlorothiazolo[5,4-b]pyridine (0.5 g, 2.0 mmol) and 3-(trifluoromethyl)cyclobutan-1-one (0.55 g, 4.01 mmol) in THF (25 mL) at −78° C. under a N₂ atmosphere was added n-butyllithium in hexanes (2.5 M, 1.6 mL, 4.0 mmol) dropwise. The reaction mixture was stirred for 5 min at −78° C. Then, the reaction mixture was diluted with satd. aq. NH₄Cl (25 mL) and extracted with EtOAc (2×25 mL). The combined organic extracts were washed with H₂O (50 mL) and dried over Na₂SO₄. The mixture was filtered and concentrated. The crude material was purified by chromatography, eluting with a gradient of 5%-15% EtOAc in hexane, to provide cis-1-(5-chlorothiazolo[5,4-b]pyridin-2-yl)-3-(trifluoromethyl)cyclobutan-1-ol (0.2 g, 0.65 mmol, 32% yield). m/z (ESI): 309.0(M+H)⁺. ¹H NMR (401 MHz, DMSO-d₆) δ 8.45 (d, J=8.5 Hz, 1H), 7.67 (d, J=8.6 Hz, 1H), 7.36 (s, 1H), 3.28-3.18 (m, 1H), 2.83 (ddd, J=10.0, 8.5, 2.7 Hz, 2H), 2.58 (td, J=9.7, 2.8 Hz, 2H).

Step 2: cis-1-(5-(2-methyl-2H-pyrazolo[3,4-b]pyridin-5-yl)[1,3]thiazolo[5,4-b]pyridin-2-yl)-3-(trifluoromethyl)cyclobutanol. A stirred solution of cis-1-(5-chlorothiazolo[5,4-b]pyridin-2-yl)-3-(trifluoromethyl)cyclobutan-1-ol (200 mg, 0.65 mmol), 2-methyl-5-(4,4,5,5-tetramethyl-1,3,2-dioxaborolan-2-yl)-2H-pyrazolo[3,4-b]pyridine (252 mg, 0.97 mmol) and K₂CO₃ (269 mg, 1.94 mmol) in 1,4-dioxane (7.2 mL) and H₂O (0.8 mL) was purged with N₂ gas for 5 min. Then, SPhos Pd G3 (51 mg, 0.065 mmol) was added. The reaction mixture was stirred at 110° C. for 2 h. Then, the reaction mixture was diluted with EtOAc (50 mL), filtered through celite, and washed with satd. aq. NH₄Cl (50 mL). The organic layer was dried over Na₂SO₄, filtered, and concentrated. The crude material was purified by reverse-phase preparative HPLC, eluting with a linear gradient of 5%-95% ACN in (0.1% NH₃) H₂O using a flow rate of 15 mL/min, to provide Compound C74C (0.145 g, 0.36 mmol, 55% yield). m/z (ESI): 406.2 (M+H)⁺. ¹H NMR (400 MHz, DMSO-d₆) δ 9.40 (d, J=2.3 Hz, 1H), 8.97 (d, J=2.3 Hz, 1H), 8.57 (s, 1H), 8.50 (d, J=8.6 Hz, 1H), 8.28 (d, J=8.6 Hz, 1H), 7.30 (s, 1H), 4.25 (s, 3H), 3.29-3.22 (m, 1H), 2.87 (td, J=8.9, 2.8 Hz, 2H), 2.59 (dd, J=12.5, 9.4 Hz, 2H).

Example 1-11: cis-3-methoxy-1-(2-(2-methyl-2H-pyrazolo[3,4-b]pyridin-5-yl)thieno[2,3-d]pyrimidin-6-yl)cyclobutanol C94C

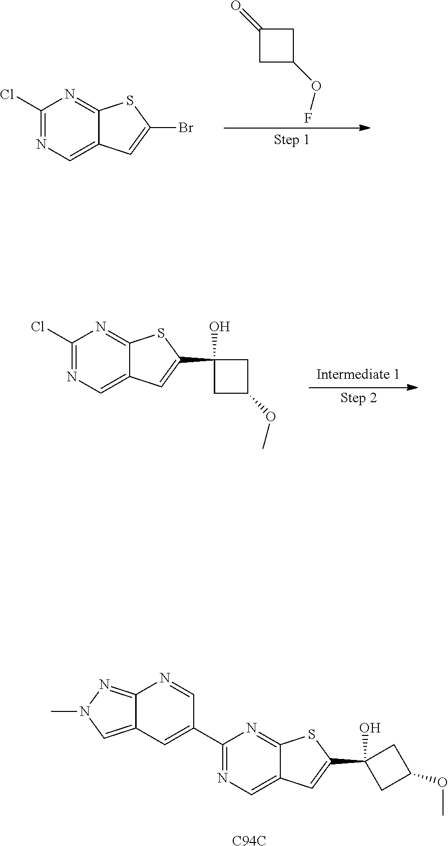

Step 1: cis-1-(5-chlorothiazolo[5,4-b]pyridin-2-yl)-3-(trifluoromethyl)cyclobutan-1-ol. To a solution of 6-bromo-2-chlorothieno[2,3-d]pyrimidine (0.150 g, 0.601 mmol) in toluene (30 mL) at −78° C. under a N₂ atmosphere was added n-butyllithium in hexanes (2.5 M, 0.481 mL, 1.20 mmol) dropwise. After 10 min, a solution of 3-methoxycyclobutan-1-one (0.090 g, 0.902 mmol) in toluene (5 mL) was added dropwise. The reaction mixture was stirred at −78° C. for 10 min. Then, the reaction mixture was diluted with satd. aq. NH₄Cl and extracted with EtOAc (2×20 mL). The combined organic extracts were washed with brine (20 mL) and dried over Na₂SO₄. The mixture was filtered and concentrated in vacuo. The crude material was purified by chromatography, eluting with a gradient of 5-40% EtOAc in hexane, to provide cis-1-(2-chlorothieno[2,3-d]pyrimidin-6-yl)-3-methoxycyclobutan-1-ol. m/z (ESI): 271.0 (M+H)⁺. ¹H NMR (400 MHz, DMSO-d₆) δ (ppm) 9.12 (s, 1H), 7.50 (s, 1H), 6.63 (s, 1H), 3.82 (p, J=7.2 Hz, 1H), 3.19 (s, 3H), 2.90-2.77 (m, 2H), 2.41-2.33 (m, 2H).

Step 2: cis-3-methoxy-1-(2-(2-methyl-2H-pyrazolo[3,4-b]pyridin-5-yl)thieno[2,3-d]pyrimidin-6-yl)cyclobutanol. A stirred solution of cis-1-(2-chlorothieno[2,3-d]pyrimidin-6-yl)-3-methoxycyclobutan-1-ol (0.15 g, 0.554 mmol), Intermediate 1 (0.215 g, 0.831 mmol), and K₂CO₃ (0.229 g, 1.662 mmol) in 1,4 dioxane (4.50 mL) and water (0.45 mL) was degassed with N₂ for 5 minutes before SPhos Pd G3 (0.043 g, 0.055 mmol) was added. The reaction mixture was stirred at 100° C. for 2 h. Then, the reaction mixture was diluted with water (25 mL) and extracted with EtOAc (3×50 mL). The combined organic extracts were washed with satd. aq. NaCl (25 mL), dried over Na₂SO₄, filtered, and concentrated. The crude material was purified by reverse-phase preparative HPLC, eluting with a linear gradient of (0.1% ammonia) water in ACN, to provide cis-3-methoxy-1-(2-(2-methyl-2H-pyrazolo[3,4-b]pyridin-5-yl)thieno[2,3-d]pyrimidin-6-yl)cyclobutanol (84 mg, 0.229 mmol, 41.3% yield). m/z (ESI): 368.0 (M+H)⁺. ¹H NMR (400 MHz, DMSO-d₆) δ 9.64 (d, J=2.4 Hz, 1H), 9.28 (s, 1H), 9.23 (d, J=2.0 Hz, 1H), 8.59 (s, 1H), 7.48 (s, 1H), 6.55 (s, 1H), 4.25 (s, 3H), 3.89-3.79 (m, 1H), 3.21 (s, 3H), 2.92-2.84 (m, 2H), 2.42-2.31 (m, 2H).

### Alternate conditions in reference to Example 1-1

(1) To a stirring solution of product from Step 2 (1 equiv.) in THF (2 mL) was added NaHMDS (483 μL, 0.48 mmol) at 0° C. under an atmosphere of N₂. The mixture was stirred at 0° C. for 0.5 h before iodoethane (188 mg, 1.21 mmol) was transferred at 0° C. The reaction was warmed to 20° C. and stirred for 12 h before the mixture was quenched by addition of sat. aq. NH₄Cl (20 mL). The aqueous phase was extracted with EtOAc (4×20 mL). The combined organic phase was washed with sat. NaCl (50 mL), dried over Na₂SO₄, filtered and concentrated in vacuo. The crude material was absorbed onto a plug of silica gel and purified by column chromatography, eluting with a gradient of 0% to 100% EtOAc in pet. ether, to provide the product.
(2) 5 mol % Pd(PPh₃)₄ and Na₂CO₃ was used as the catalyst and base, instead of Pd(dppf)Cl₂ and K₂CO₃.
(3) Deprotection: Following Step 2, where Intermediate 4 was used, the product was dissolved in dichloromethane (2.5 mL) and TFA (41.8 μL, 0.54 mmol) and stirred at 20° C. for 4 h. The reaction mixture was concentrated in vacuo. The crude material was absorbed onto a plug of silica gel and purified by column chromatography, eluting with a gradient of 0% to 80% EtOAc in pet. ether, to provide the product.
(4) To a stirring solution of product from Step 1 (1 equiv.) in THF (5 mL) was added trimethyl(trifluoromethyl)silane (0.72 g, 5.06 mmol) and TBAF (3 mL, 3.00 mmol) at 0° C. Then, the mixture was stirred at 20° C. for 2 h. The residue was diluted with H₂O (20 mL) and extracted with EtOAc (30 mL×2). The combined organic layers were washed with brine (10 mL×2), dried over Na₂SO₄, filtered, and concentrated under reduced pressure to give a crude residue. The crude residue was purified by chromatography, eluting with a gradient of 0-6% EtOAc in pet. ether, to provide the product.
(5) 10 mol % o SPhos Pd G3 was used as the catalyst, instead of Pd(dppf)Cl₂.
(6) Deprotection: Following Step 2, where Intermediate 4 was used, the product was dissolved in THE (5 mL) at 0° C. Conc. HCl (2 mL) was added, and the reaction was stirred for 5 h at rt. The reaction mass was quenched by addition of satd. aq. NaHCO₃ (20 mL) and extracted with EtOAc (2×25 mL). The combined organic layers were washed with brine (10 mL×2), dried over Na₂SO₄, filtered, and concentrated under reduced pressure. The crude material was purified by reverse-phase preparative HPLC, eluting with a gradient of 353795% ACN in (0.1 NH₃) H₂ using a flow rate of 15 mL/min, to provide the product.
(7) Step 1 was performed in toluene instead of THF.
(8) Step 1 was perfomed in DCM instead of THF.

Compounds in Table 1-1 were prepared following the procedure described in Group 1, using appropriate starting materials (e.g., aldehyde/ketones and boronic esters). All starting materials are commercially available or are described in the Intermediates section above.

TABLE 1-1

| | | | Reaction |
|---|---|---|---|
| Ex. # | Chemical Structure & Name | LCMS: (ESI + ve ion) m/z; NMR | Comments |
| B16 | 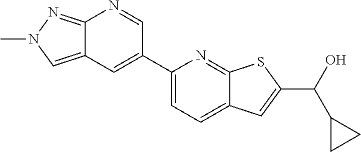 [SMILES: Cn1cc2cc(-c3ccc4cc(C(O)C5CC5)sc4n3)cnc2n1] | 337.1; ¹H NMR (400 MHz, DMSO- d₆) δ 9.38 (d, J = 2.3 Hz, 1H), 8.91 (d, J = 2.3 Hz, 1H), 8.54 (s, 1H), 8.26 (d, J = 8.4 Hz, 1H), 8.08 (d, J = 8.4 Hz, 1H), 7.33 (s, 1H), 5.88 (d, J = 5.0 Hz, 1H), 4.35-4.30 (m, 1H), 4.23 (s, 3H), 1.35-1.15 (m, 1H), 0.58-0.55 (m, 2H), 0.50-0.48 (m, 2H) | |
| | cyclopropyl(6-(2-methyl-2H- | | |
| | pyrazolo[3,4-b]pyridin-5- | | |
| | yl)thieno[2,3-b]pyridin-2- | | |
| | yl)methanol | | |
| B16S | 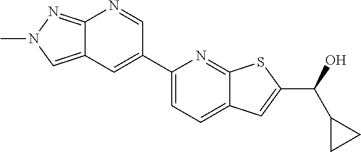 [SMILES: Cn1cc2cc(-c3ccc4cc([C@@H](O)C5CC5)sc4n3)cnc2n1] | 337.1; ¹H NMR (400 MHz, DMSO- d₆) δ 9.38 (d, J = 2.0 Hz, 1H), 8.91 (d, J = 2.0 Hz, 1H), 8.54 (s, 1H), 8.26 (d, J = 8.4 Hz, 1H), 8.08 (d, J = 8.4 Hz, 1H), 7.32 (s, 1H), 5.89 (br s, 1H), 4.31 (d, J = 7.6 Hz, 1H), 4.24 (s, 3H), 1.30-1.14 (m, 1H), 0.62-0.43 (m, 4H) | Peak 1/SFC: Chiralpak IH-3, 50 × 4.6 mm 3 μm; Mobile phase: 5-50% EtOH (0.2% NH₃; 7 M in MeOH) in CO₂ using a flow rate of 3.4 mL/min. |
| | (S)-cyclopropyl(6-(2-methyl-2H- | | |
| | pyrazolo[3,4-b]pyridin-5- | | |
| | yl)thieno[2,3-b]pyridin-2- | | |
| | yl)methanol | | |
| B16R | 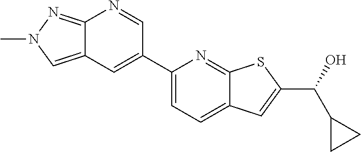 [SMILES: Cn1cc2cc(-c3ccc4cc([C@H](O)C5CC5)sc4n3)cnc2n1] | 337.1; ¹H NMR (400 MHz, DMSO- d₆) δ 9.38 (d, J = 2.3 Hz, 1H), 8.91 (d, J = 2.3 Hz, 1H), 8.54 (s, 1H), 8.26 (d, J = 8.4 Hz, 1H), 8.08 (d, J = 8.4 Hz, 1H), 7.33 (s, 1H), 5.88 (br s, 1H), 4.31 (d, J = 7.6 Hz, 1H), 4.24 (s, 3H), 1.29-1.16 (m, 1H), 0.60-0.53 (m, 2H), 0.52-0.44 (m, 2H | Peak 2/SFC: Chiralpak IH-3, 50 × 4.6 mm 3 μm; Mobile phase: 5-50% EtOH (0.2% NH₃; 7 M in MeOH) in CO₂ using a flow rate of 3.4 mL/min. |
| | (R)-cyclopropyl(6-(2-methyl- | | |
| | 2H-pyrazolo[3,4-b]pyridin-5- | | |
| | yl)thieno[2,3-b]pyridin-2- | | |
| | yl)methanol | | |
| B19RR | 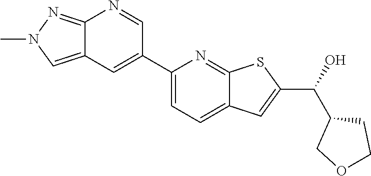 [SMILES: Cn1cc2cc(-c3ccc4cc([C@H](O)[C@@H]5CCOC5)sc4n3)cnc2n1] | 367.0; ¹H NMR (500 MHz, DMSO- d₆) δ 9.44 (d, J = 2.3 Hz, 1H), 9.01 (d, J = 2.3 Hz, 1H), 8.54 (s, 1H), 8.16 (s, 1H), 7.66 (d, J = 1.8 Hz, 1H), 7.11 (d, J = 1.0 Hz, 1H), 6.66 (d, J = 2.0 Hz, 1H), 5.85 (d, J = 4.8 Hz, 1H), 5.1-5.0 (m, 1H), 4.24 (s, 3H), 3.90 (s, 3H), 1.50 (d, J = 6.4 Hz, 3H) | Peak 1/SFC: Chiralpak AY, 21 × 250 mm 5 μm; mobile phase of 50% MeOH with DEA using a flowrate of 80 mL/min. Peak 1/SFC: Chiralpak AS, 21 ×x 250 mm 5 μm; mobile phase of 50% MeOH with DEA using a flowrate of 80 mL/min. |
| | (R)-(6-(2-methyl-2H- | | |
| | pyrazolo[3,4-b]pyridin-5- | | |
| | yl)thieno[2,3-b]pyridin-2- | | |
| | yl)((3R)-tetrahydro-3- | | |
| | furanyl)methanol | | |
| B19SR | 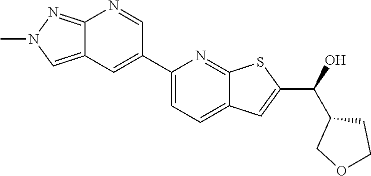 [SMILES: Cn1cc2cc(-c3ccc4cc([C@@H](O)[C@@H]5CCOC5)sc4n3)cnc2n1] | 367.0; ¹H NMR (500 MHz, DMSO- d₆) δ 9.44 (d, J = 2.3 Hz, 1H), 9.01 (d, J = 2.3 Hz, 1H), 8.54 (s, 1H), 8.16 (s, 1H), 7.66 (d, J = 1.8 Hz, 1H), 7.11 (d, J = 1.1 Hz, 1H), 6.66 (d, J = 2.0 Hz, 1H), 5.85 (d, J = 4.8 Hz, 1H), 5.1-5.0 (m, 1H), 4.24 (s, 3H), 3.90 (s, 3H), 1.50 (d, J = 6.5 Hz, 3H) | Peak 2/SFC: Chiralpak AY, 21 × 250 mm 5 μm; mobile phase of 50% MeOH with DEA using a flowrate of 80 mL/min. |
| | (S)-(6-(2-methyl-2H- | | |
| | pyrazolo[3,4-b]pyridin-5- | | |
| | yl)thieno[2,3-b]pyridin-2- | | |
| | yl)((3R)-tetrahydro-3- | | |
| | furanyl)methanol | | |
| B19 (RS)S | 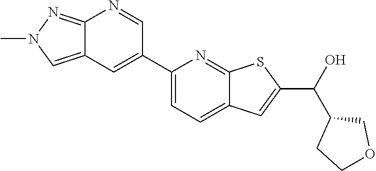 [SMILES: Cn1cc2cc(-c3ccc4cc(C(O)[C@H]5CCOC5)sc4n3)cnc2n1] | 367.2; ¹H NMR (400 MHz, CHLOROFORM-d) δ 9.35 (s, 1H), 8.75 (d, J = 1.0 Hz, 1H), 8.1-8.0 (m, 2H), 7.78 (d, J = 8.2 Hz, 1H), 7.16 (s, 1H), 4.95 (d, J = 7.5 Hz, 1H), 4.30 (s, 3H), 3.99 (dt, J = 5.4, 7.9 Hz, 1H), 3.9-3.7 (m, 2H), 3.7- 3.6 (m, 1H), 2.9-2.7 (m, 1H), 2.2- 2.0 (m, 2H), 1.3-1.2 (m, 1H). | Peak 1/SFC: Chiralpak AY, 21 × 250 mm 5 μm; mobile phase of 50% MeOH with DEA using a flowrate of 80 mL/min. Peak 2/SFC: Chiralpak AS, 21 × 250 mm 5 μm; mobile phase of 50% MeOH with DEA using a flowrate of 80 mL/min. |
| | (6-(2-methyl-2H-pyrazolo[3,4- | | |
| | b]pyridin-5-yl)thieno[2,3- | | |
| | b]pyridin-2-yl)((3S)-tetrahydro- | | |
| | 3-furanyl)methanol | | |
| B22 | 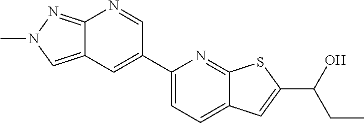 [SMILES: CCC(O)c1cc2ccc(-c3cnc4nn(C)cc4c3)nc2s1] | 325.0; ¹H NMR (400 MHz, DMSO- d₆) δ 9.37 (d, J = 2.4 Hz, 1H), 8.90 (d, J = 2.4 Hz, 1H), 8.53 (s, 1H), 8.24 (d, J = 8.4 Hz, 1H), 8.07 (d, J = 8.4 Hz, 1H), 7.27 (s, 1H), 5.84 (d, J = 4.8 Hz, 1H), 4.84-4.80 (m, 1H), 4.24 (s, 3H), 1.83-1.76 (m, 2H), 0.95-0.91 (m, 3H) | |
| | 1-(6-(2-methyl-2H-pyrazolo[3,4- | | |
| | b]pyridin-5-yl)thieno[2,3- | | |
| | b]pyridin-2-yl)-1-propanol | | |
| B23 | 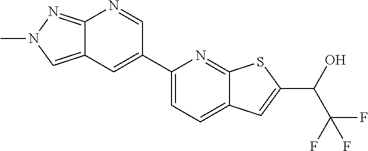 [SMILES: Cn1cc2cc(-c3ccc4cc(C(O)C(F)(F)F)sc4n3)cnc2n1] | 365.0; ¹H NMR (400 MHz, DMSO- d₆) δ 9.39 (d, J = 2.4 Hz, 1 H), 8.94 (d, J = 2.4 Hz, 1 H), 8.55 (s, 1 H), 8.38 (d, J = 8.4 Hz, 1 H), 8.15 (d, J = 8.4 Hz, 1 H), 7.58 (s, 1 H), 7.54 (br d, J = 3.6 Hz, 1 H), 5.78-5.65 (m, 1 H), 4.24 (s, 3 H) | Alternative condition (4) was used with DMF as the electrophile in Step 1 |
| | 2,2,2-trifluoro-1-(6-(2-methyl- | | |
| | 2H-pyrazolo[3,4-b]pyridin-5- | | |
| | yl)thieno[2,3-b]pyridin-2- | | |
| | yl)ethanol | | |
| B23S | 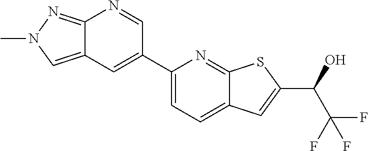 [SMILES: Cn1cc2cc(-c3ccc4cc([C@@H](O)C(F)(F)F)sc4n3)cnc2n1] | 365.1; ¹H NMR (400 MHz, DMSO- d₆) δ 9.43-9.37 (m, 1 H), 8.99- 8.91 (m, 1H), 8.55 (s, 1H), 8.38 (d, J = 8.4 Hz, 1H), 8.15 (br d, J = 8.4 Hz, 1H), 7.73-7.61 (m, 1H), 7.58 (s, 1H), 5.79-5.64 (m, 1H), 4.24 (s, 3H) ¹⁹F NMR (377 MHz, DMSO-d₆) δ- 76.80 (s, 3F) | Alternative condition (4) was used with DMF as the electrophile in Step 1 Peak 1/SFC: Chiralpak IH-3, 100 × 4.6 mm 3 μm; Mobile phase: 50% IPA (0.2% NH₃ (7 M in MeOH)) in CO₂ using a flow rate of 4.0 mL/min. |
| | (1S)-2,2,2-trifluoro-1-(6-(2- | | |
| | methyl-2H-pyrazolo[3,4- | | |
| | b]pyridin-5-yl)thieno[2,3- | | |
| | b]pyridin-2-yl)ethanol | | |
| B23R | 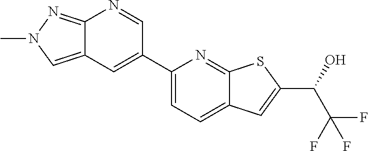 [SMILES: Cn1cc2cc(-c3ccc4cc([C@H](O)C(F)(F)F)sc4n3)cnc2n1] | 365.1; ¹H NMR (400 MHz, DMSO- d₆) δ 9.39 (d, J = 2.3 Hz, 1H), 8.94 (d, J = 2.4 Hz, 1H), 8.55 (s, 1H), 8.38 (d, J = 8.4 Hz, 1H), 8.15 (d, J = 8.4 Hz, 1H), 7.58 (s, 1H), 7.55 (d, J = 5.8 Hz, 1H), 5.79-5.62 (m, 1H), 4.24 (s, 3H) ¹⁹F NMR (377 MHz, DMSO-d₆) δ- 76.80 (s, 3 F) | Alternative condition (4) was used with DMF as the electrophile in Step 1 Peak 2/SFC: Chiralpak IH-3, 100 × 4.6 mm 3 μm; Mobile phase: 50% IPA (0.2% NH₃ (7 M in MeOH)) in CO₂ using a flow rate of 4.0 mL/min. |
| | (1R)-2,2,2-trifluoro-1-(6-(2- | | |
| | methyl-2H-pyrazolo[3,4- | | |
| | b]pyridin-5-yl)thieno[2,3- | | |
| | b]pyridin-2-yl)ethanol | | |
| B24 | 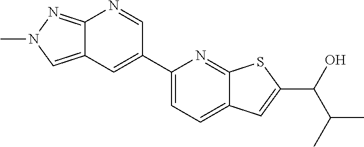 [SMILES: CC(C)C(O)c1cc2ccc(-c3cnc4nn(C)cc4c3)nc2s1] | 339.0; ¹H NMR (DMSO-d₆) δ: 9.37 (d, J = 2.3 Hz, 1H), 8.90 (d, J = 2.1 Hz, 1H), 8.53 (s, 1H), 8.25 (d, J = 8.4 Hz, 1H), 8.07 (d, J = 8.4 Hz, 1H), 7.26 (s, 1H), 5.85 (d, J = 4.5 Hz, 1H), 4.64 (t, J = 5.2 Hz, 1H), 4.24 (s, 3H), 2.01-1.88 (m, 1H), 0.96 (d, J = 6.8 Hz, 3H), 0.89 (d, J = 6.8 Hz, 3H) | |
| | 2-methyl-1-(6-(2-methyl-2H- | | |
| | pyrazolo[3,4-b]pyridin-5- | | |
| | yl)thieno[2,3-b]pyridin-2- | | |
| | yl)propan-1-ol | | |
| B24R | 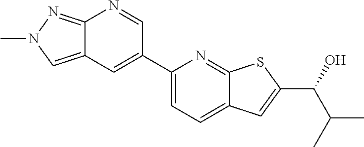 [SMILES: CC(C)[C@@H](O)c1cc2ccc(-c3cnc4nn(C)cc4c3)nc2s1] | 339.2; ¹H NMR (400 MHz, DMSO- d₆) δ 9.38 (d, J = 2.3 Hz, 1 H), 8.91 (d, J = 2.3 Hz, 1 H), 8.55 (s, 1 H), 8.26 (d, J = 8.5 Hz, 1 H), 8.08 (d, J = 8.5 Hz, 1 H), 7.27 (s, 1 H), 5.87 (d, J = 4.6 Hz, 1 H), 4.65 (t, J = 5.2 Hz, 1 H), 4.24 (s, 3 H), 2.00- 1.91 (m, 1 H), 0.99-0.87 (m, 6 H) | Peak 1/SFC: Chiralcel OX, 21 × 150 mm 5 μm; mobile phase: 60% CO₂ : 40% MeOH(0.2 DEA, v/v) using a flowrate of 150 mL/min |
| | (1R)-2-methyl-1-(6-(2-methyl- | | |
| | 2H-pyrazolo[3,4-b]pyridin-5- | | |
| | yl)thieno[2,3-b]pyridin-2-yl)-1- | | |
| | propanol | | |
| B24S | 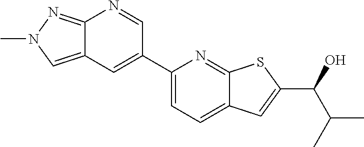 [SMILES: CC(C)[C@H](O)c1cc2ccc(-c3cnc4nn(C)cc4c3)nc2s1] | 339.2; ¹H NMR (400 MHz, DMSO- d₆) δ 9.38 (d, J = 2.3 Hz, 1 H), 8.91 (d, J = 2.3 Hz, 1 H), 8.54 (s, 1 H), 8.26 (d, J = 8.5 Hz, 1 H), 8.08 (d, J = 8.5 Hz, 1 H), 7.26 (s, 1 H), 5.87 (d, J = 4.6 Hz, 1 H), 4.65 (t, J = 5.2 Hz, 1 H), 4.24 (s, 3 H), 2.00-1.91 (m, 1 H), 0.99-0.88 (m, 6 H) | Peak 2/SFC: Chiralcel OX, 21 x 150 mm 5μm; mobile phase: 60% CO_{2:} 40% MeOH(0.2 DEA, v/v) using a flowrate of 150 mL/min |
| | (1S)-2-methyl-1-(6-(2-methyl- | | |
| | 2H-pyrazolo[3,4-b]pyridin-5- | | |
| | yl)thieno[2,3-b]pyridin-2-yl)-1- | | |
| | propanol | | |
| B25 | 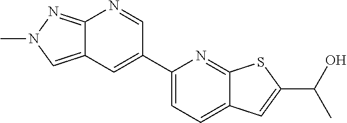 [SMILES: CC(O)c1cc2ccc(-c3cnc4nn(C)cc4c3)nc2s1] | 311.0; ¹H NMR (400 MHz, DMSO- d₆) δ 9.38 (d, J = 2.4 Hz, 1H), 8.91 (d, J = 2.4 Hz, 1H), 8.54 (s, 1H), 8.24 (d, J = 8.4 Hz, 1H), 8.08 (d, J = 8.8 Hz, 1H), 7.26 (s, 1H), 5.85 (d, J = 4.8 Hz, 1H), 5.10-5.04 (m, 1H), 4.24 (s, 3H), 1.51 (d, J = 6.4 Hz, 3H) | |
| | 1-(6-(2-methyl-2H-pyrazolo[3,4- | | |
| | b]pyridin-5-yl)thieno[2,3- | | |
| | b]pyridin-2-yl)ethanol | | |
| B27 | 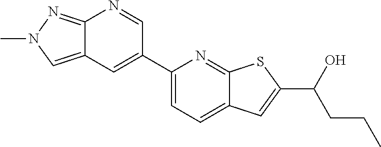 [SMILES: CCCC(O)c1cc2ccc(-c3cnc4nn(C)cc4c3)nc2s1] | 339.0; ¹H NMR (400 MHz, DMSO- d₆) δ 9.37 (d, J = 2.0 Hz, 1H), 8.90 (d, J = 2.0 Hz, 1H), 8.53 (s, 1H), 8.24 (d, J = 8.4 Hz, 1H), 8.07 (d, J = 8.4 Hz, 1H), 7.27 (s, 1H), 5.83 (d, J = 4.8 Hz, 1H), 4.91-4.87 (m, 1H), 4.24 (s, 3H), 1.77-1.73 (m, 2H), 1.46-1.35 (m, 2H), 0.92 (t, J = 7.2 Hz, 3H) | |
| | 1-(6-(2-methyl-2H-pyrazolo[3,4- | | |
| | b]pyridin-5-yl)thieno[2,3- | | |
| | b]pyridin-2-yl)-1-butanol | | |
| B28 | 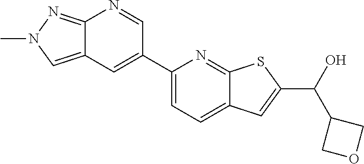 [SMILES: Cn1cc2cc(-c3ccc4cc(C(O)C5COC5)sc4n3)cnc2n1] | 353.0; ¹H NMR (400 MHz, DMSO- d₆) δ 9.37 (d, J = 2.3 Hz, 1H), 8.90 (d, J = 2.3 Hz, 1H), 8.54 (s, 1H), 8.25 (d, J = 8.5 Hz, 1H), 8.08 (d, J = 8.4 Hz, 1H), 7.29 (d, J = 0.6 Hz, 1H), 6.24 (br d, J = 4.1 Hz, 1H), 5.18 (br dd, J = 7.2, 3.3 Hz, 1H), 4.67-4.55 (m, 3H), 4.50 (t, J = 6.2 Hz, 1H), 4.23 (s, 3H), 3.39- 3.31 (m, 1H) | |
| | (6-(2-methyl-2H-pyrazolo[3,4- | | |
| | b]pyridin-5-yl)thieno[2,3- | | |
| | b]pyridin-2-yl)(oxetan-3- | | |
| | yl)methanol | | |
| B28R | 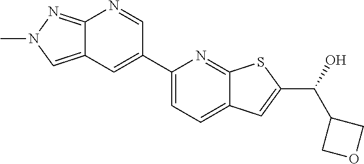 [SMILES: Cn1cc2cc(-c3ccc4cc([C@H](O)C5COC5)sc4n3)cnc2n1] | 353.1; ¹H NMR (400 MHz, DMSO- d₆) δ 9.37 (d, J = 2.2 Hz, 1H), 8.90 (d, J = 2.2 Hz, 1H), 8.53 (s, 1H), 8.25 (d, J = 8.3 Hz, 1H), 8.08 (d, J = 8.3 Hz, 1H), 7.29 (s, 1H), 6.23 (s, 1H), 5.18 (d, J = 7.5 Hz, 1H), 4.67- 4.56 (m, 3H), 4.50 (t, J = 6.1 Hz, 1H), 4.24 (s, 3H), 3.34 (br d, J = 7.6 Hz, 1 H) | Peak 1/SFC: Chiralpak IH-3, 50 × 4.6 mm 3 μm; Mobile phase: 50-55% EtOH (0.1% NH₃; 7 M in MeOH) in CO₂ using a flow rate of 4.0 mL/min. |
| | (R)-(6-(2-methyl-2H- | | |
| | pyrazolo[3,4-b]pyridin-5- | | |
| | yl)thieno[2,3-b]pyridin-2-yl)(3- | | |
| | oxetanyl)methanol | | |
| B28S | 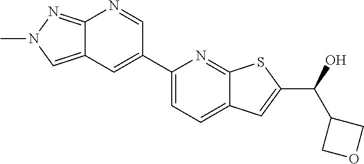 [SMILES: Cn1cc2cc(-c3ccc4cc([C@@H](O)C5COC5)sc4n3)cnc2n1] | 353.1; ¹H NMR (400 MHz, DMSO- d₆) δ 9.37 (d, J = 2.3 Hz, 1H), 8.90 (d, J = 2.3 Hz, 1H), 8.53 (s, 1H), 8.25 (d, J = 8.5 Hz, 1H), 8.08 (d, J = 8.5 Hz, 1H), 7.29 (s, 1H), 6.23 (s, 1H), 5.18 (d, J = 7.6 Hz, 1H), 4.67- 4.56 (m, 3H), 4.52-4.48 (m, 1H), 4.24 (s, 3H), 3.34 (br d, J = 7.6 Hz, 1H) | Peak 2/SFC: Chiralpak IH-3, 50 × 4.6 mm 3 μm; Mobile phase: 50-55% EtOH (0.1% NH₃; 7 M in MeOH) in CO₂ using a flow rate of 4.0 mL/min. |
| | (S)-(6-(2-methyl-2H- | | |
| | pyrazolo[3,4-b]pyridin-5- | | |
| | yl)thieno[2,3-b]pyridin-2-yl)(3- | | |
| | oxetanyl)methanol | | |
| B29 | 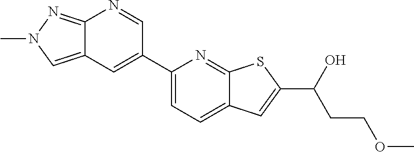 | 355.1; ¹H NMR (400 MHz, DMSO- d₆) δ 9.38 (d, J = 2.4 Hz, 1H), 8.91 (d, J = 2.2 Hz, 1H), 8.54 (s, 1H), 8.25 (d, J = 8.3 Hz, 1H), 8.09 (d, J = 8.3 Hz, 1H), 7.28 (s, 1H), 5.96 (d, J = 5.0 Hz, 1H), 5.04-4.95 (m, 1H), 4.24 (s, 3H), 3.52 (td, J = 6.7, 9.2 Hz, 1H), 3.40 (td, J = 6.0, 9.4 Hz, 1H), 3.25 (s, 3H), 2.06- 1.93 (m, 2H) | |
| | 3-methoxy-1-(6-(2-methyl-2H- | | |
| | pyrazolo[3,4-b]pyridin-5- | | |
| | yl)thieno[2,3-b]pyridin-2- | | |
| | yl)propan-1-ol | | |
| B31 | 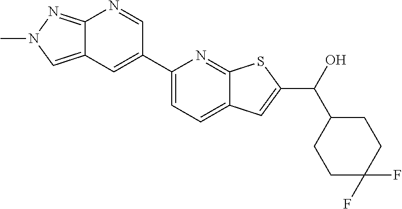 [SMILES: Cn1cc2cc(-c3ccc4cc(C(O)C5CCC(F)(F)CC5)sc4n3)cnc2n1] | 415.1; ¹H NMR (400 MHz, DMSO- d₆) δ 9.37 (d, J = 1.8 Hz, 1H), 8.90 (d, J = 1.9 Hz, 1H), 8.54 (s, 1H), 8.26 (d, J = 8.4 Hz, 1H), 8.08 (d, J = 8.4 Hz, 1H), 7.29 (s, 1H), 6.02 (d, J = 4.5 Hz, 1H), 4.75 (br t, J = 5.2 Hz, 1H), 4.24 (s, 3H), 2.09-1.90 (m, 3H), 1.88-1.68 (m, 3H), 1.58 (br d, J = 12.1 Hz, 1H), 1.47-1.28 (m, 2H) | |
| | (4,4-difluorocyclohexyl)(6-(2- | | |
| | methyl-2H-pyrazolo[3,4- | | |
| | b]pyridin-5-yl)thieno[2,3- | | |
| | b]pyridin-2-yl)methanol | | |
| B32 | 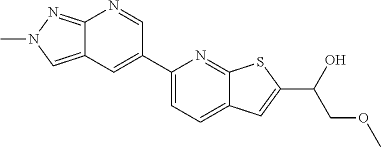 | 341.0; ¹H NMR (400 MHz, DMSO- d₆) δ 9.38 (d, J = 2.0 Hz, 1H), 8.92 (d, J = 2.4 Hz, 1H), 8.54 (s, 1H), 8.27 (d, J = 8.4 Hz, 1H), 8.09 (d, J = 8.4 Hz, 1H), 7.33 (s, 1H), 6.10 (d, J = 4.8 Hz, 1H), 5.08-5.04 (m, 1H), 4.24 (s, 3H), 3.60-3.56 (m, 2H), 3.33 (s, 3H) | |
| | 2-methoxy-1-(6-(2-methyl-2H- | | |
| | pyrazolo[3,4-b]pyridin-5- | | |
| | yl)thieno[2,3-b]pyridin-2- | | |
| | yl)ethanol | | |
| B33 | 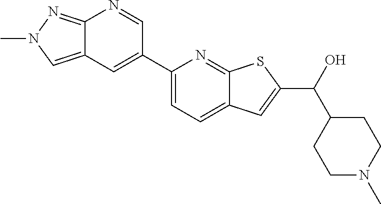 | 394.1; ¹H NMR (400 MHz, DMSO- d₆) δ 10.17 (s, 1H), 9.44 (s, 1H), 9.09 (s, 1H), 8.67 (s, 1H), 8.30 (d, J = 8.4 Hz, 1H), 8.13 (d, J = 8.4 Hz, 1H), 7.33 (s, 1H), 4.73 (d, J = 6.4 Hz, 1H), 4.27 (s, 3H), 3.38 (t, J = 14.4 Hz, 2H), 2.95-2.8 (m, 2H), 2.67 (d, J = 4.4 Hz, 3H), 2.08- 1.92 (m, 1H), 1.92-1.80 (m, 1H), 1.73-1.55 (m, 3H) | |
| | (1-methyl-4-piperidinyl)(6-(2- | | |
| | methyl-2H-pyrazolo[3,4- | | |
| | b]pyridin-5-yl)thieno[2,3- | | |
| | b]pyridin-2-yl)methanol | | |
| B34 | 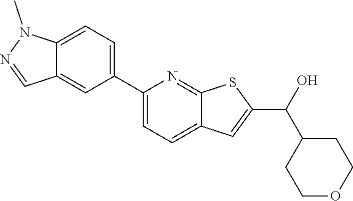 [SMILES: Cn1ncc2cc(-c3ccc4cc(C(O)C5CCOCC5)sc4n3)ccc21] | 380.0; ¹H NMR (400 MHz, DMSO- d₆) δ 8.55 (s, 1H), 8.35-8.25 (m, 2H), 8.20 (s, 1H), 8.04 (d, J = 8.4 Hz, 1H), 7.75 (d, J = 7.2 Hz, 1H), 7.25 (s, 1H), 5.94 (d, J = 4.4 Hz, 1H), 4.67-4.64 (m, 1H), 4.09 (s, 3H), 3.94-3.77 (m, 2H), 3.35-3.18 (m, 2H), 1.95-1.85 (m, 1H), 1.76 (s, 1H), 1.44-1.23 (m, 3H) | |
| | (6-(1-methyl-1H-indazol-5- | | |
| | yl)thieno[2,3-b]pyridin-2- | | |
| | yl)(tetrahydro-2H-pyran-4- | | |
| | yl)methanol | | |
| B34S | 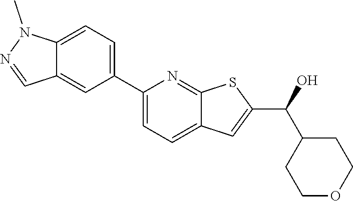 [SMILES: Cn1ncc2cc(-c3ccc4cc([C@@H](O)C5CCOCC5)sc4n3)ccc21] | 380.1; ¹H NMR (400 MHz, DMSO- d₆) δ 8.55 (s, 1H), 8.35-8.25 (m, 2H), 8.20 (s, 1H), 8.04 (d, J = 8.4 Hz, 1H), 7.75 (d, J = 9.2 Hz, 1H), 7.26 (s, 1H), 5.94 (d, J = 4.4 Hz, 1H), 4.67-4.64 (m, 1H), 4.09 (s, 3H), 3.94-3.77 (m, 2H), 3.35- 3.18 (m, 2H), 1.95-1.85 (m, 1H), 1.75 (s, 1H), 1.44-1.23 (m, 3H) | Peak 1/SFC: Chiralpak AD-3, 50 × 4.6 mm I.D., 3 μm; Mobile phase: 50% CO₂: 50% EtOH:ACN=1:1(0.1% IPAm, v/v) using a flow rate of 4 mL/min |
| | (S)-(6-(1-methyl-1H-indazol-5- | | |
| | yl)thieno[2,3-b]pyridin-2- | | |
| | yl)(tetrahydro-2H-pyran-4- | | |
| | yl)methanol | | |
| B34R | 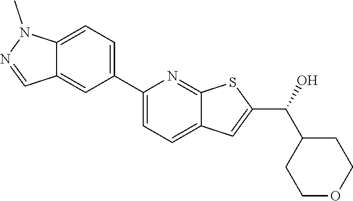 [SMILES: Cn1ncc2cc(-c3ccc4cc([C@H](O)C5CCOCC5)sc4n3)ccc21] | 380.1; ¹H NMR (400 MHz, DMSO- d₆) δ 8.55 (s, 1H), 8.35-8.25 (m, 2H), 8.20 (s, 1H), 8.04 (d, J = 8.0 Hz, 1H), 7.75 (d, J = 8.8 Hz, 1H), 7.25 (s, 1H), 5.93 (d, J = 4.4 Hz, 1H), 4.66-4.63 (m, 1H), 4.09 (s, 3H), 3.94-3.77 (m, 2H), 3.35- 3.18 (m, 2H), 1.95-1.85 (m, 1H), 1.75 (s, 1H), 1.44-1.23 (m, 3H) | Peak 2/SFC: Chiralpak AD-3, 50 × 4.6 mm I.D., 3 μm; Mobile phase: 50% CO₂: 50% EtOH:ACN = 1:1(0.1% IPAm, v/v) using a flow rate of 4 mL/min |
| | (R)-(6-(1-methyl-1H-indazol-5- | | |
| | yl)thieno[2,3-b]pyridin-2- | | |
| | yl)(tetrahydro-2H-pyran-4- | | |
| | yl)methanol | | |
| B35S | 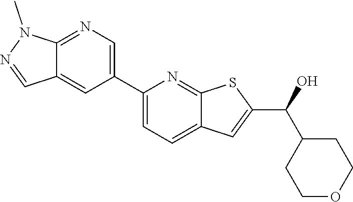 [SMILES: Cn1ncc2cc(-c3ccc4cc([C@@H](O)C5CCOCC5)sc4n3)cnc21] | 381.0; ¹H NMR (400 MHz, DMSO- d₆) δ 9.34 (d, J = 2.0 Hz, 1H), 8.94 (d, J = 2.0 Hz, 1H), 8.23-8.30 (m, 2H), 8.10 (d, J = 8.4 Hz, 1H), 7.28 (s, 1H), 5.97 (s, 1H), 4.67 (d, J = 6.8 Hz, 1H), 4.11 (s, 3H), 3.93- 3.77 (m, 2H), 3.35-3.15 (m, 2H), 1.93-1.85 (m, 1H), 1.85-1.65 (m, 1H), 1.44-1.25 (m, 3H) | Alternative condition (2) was used Peak 1/SFC Column: Chiralpak IH-3, 100 × 4.6 mm, I.D., 3 μm Mobile phase: 60% CO₂: 40% EtOH (0.1% IPAm) using a flow rate of 4 mL/min |
| | (S)-(6-(1-methyl-1H- | | |
| | pyrazolo[3,4-b]pyridin-5- | | |
| | yl)thieno[2,3-b]pyridin-2- | | |
| | yl)(tetrahydro-2H-pyran-4- | | |
| | yl)methanol | | |
| B35R | 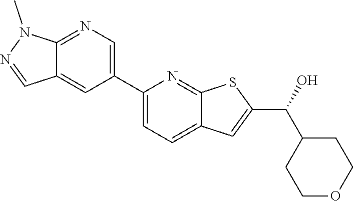 [SMILES: Cn1ncc2cc(-c3ccc4cc([C@H](O)C5CCOCC5)sc4n3)cnc21] | 381.1; ¹H NMR (400 MHz, DMSO- d₆) δ 9.34 (d, J = 2.0 Hz, 1H), 8.94 (d, J = 2.0 Hz, 1H), 8.23-8.30 (m, 2H), 8.09 (d, J = 8.4 Hz, 1H), 7.28 (s, 1H), 5.97 (d, J = 4.4 Hz, 1H), 4.68-4.65 (m, 1H), 4.11 (s, 3H), 3.90-3.81 (m, 2H), 3.27-3.23 (m, 2H), 1.84-1.74 (m, 2H), 1.39- 1.32 (m, 3H) | Alternative condition (2) was used Peak 2/SFC Column: Chiralpak IH-3, 100 × 4.6 mm, I.D., 3 μm Mobile phase: 60% CO₂: 40% EtOH (0.1% IPAm) using a flow rate of 4 mL/min |
| | (R)-(6-(1-methyl-1H- | | |
| | pyrazolo[3,4-b]pyridin-5- | | |
| | yl)thieno[2,3-b]pyridin-2- | | |
| | yl)(tetrahydro-2H-pyran-4- | | |
| | yl)methanol | | |
| B37R | 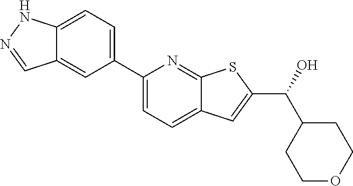 [SMILES: O[C@@H](c1cc2ccc(-c3ccc4[nH]ncc4c3)nc2s1)C1CCOCC1] | 366.0; ¹H NMR (400 MHz, DMSO- d₆) δ 13.19 (s, 1H), 8.55 (s, 1H), 8.22-8.15 (m, 3H), 8.02 (d, J = 8.0 Hz, 1H), 7.64 (d, J = 8.8 Hz, 1H), 7.25 (s, 1H), 5.94 (d, J = 4.0 Hz, 1H), 4.65 (t, J = 4.4 Hz, 1H), 3.87-3.80 (m, 2H), 3.33-3.23 (m, 2H), 1.81-1.75 (m, 2H), 1.38- 1.31 (m, 3H) | Peak 1/SFC: Chiralpak AD-3, 50 × 4.6 mm I.D., 3 μm Mobile phase: 50% CO₂: 50% EtOH(0.1% IPAm, v/v) using a flow rate of 4 mL/min |
| | (R)-(6-(1H-indazol-5- | | |
| | yl)thieno[2,3-b]pyridin-2- | | |
| | yl)(tetrahydro-2H-pyran-4- | | |
| | yl)methanol | | |
| B37S | 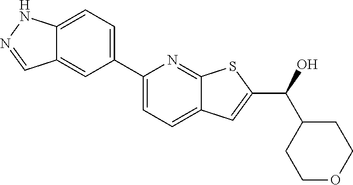 [SMILES: O[C@H](c1cc2ccc(-c3ccc4[nH]ncc4c3)nc2s1)C1CCOCC1] | 366.0; ¹H NMR (400 MHz, DMSO- d₆) δ 13.18 (s, 1H), 8.55 (s, 1H), 8.22-8.21 (m, 1H), 8.20-8.19 (m, 2H), 8.02 (d, J = 8.4 Hz, 1H), 7.65 (d, J = 8.8 Hz, 1H), 7.25 (s, 1H), 5.93 (s, 1H), 4.67-6.62 (m, 1H), 3.89-3.87 (m, 2H), 3.29-3.26 (m, 2H), 1.90-1.85 (m, 1H), 1.67- 1.85 (m, 1H), 1.40-1.35 (m, 3H) | Peak 2/SFC: Chiralpak AD-3, 50 × 4.6 mm I.D., 3 μm Mobile phase: 50% CO₂: 50% EtOH(0.1% IPAm, v/v) using a flow rate of 4 mL/min |
| | (S)-(6-(1H-indazol-5- | | |
| | yl)thieno[2,3-b]pyridin-2- | | |
| | yl)(tetrahydro-2H-pyran-4- | | |
| | yl)methanol | | |
| B38R | 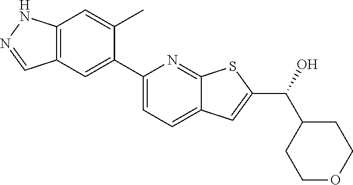 [SMILES: Cc1cc2[nH]ncc2cc1-c1ccc2cc([C@H](O)C3CCOCC3)sc2n1] | 380.1; ¹H NMR (400 MHz, DMSO- d₆) δ 13.01 (s, 1H), 8.21 (d, J = 8.3 Hz, 1 H), 8.07 (s, 1 H), 7.79 (s, 1 H), 7.54 (d, J = 8.1 Hz, 1H), 7.45 (s, 1H), 7.28 (s, 1H), 5.92 (d, J = 4.6 Hz, 1H), 4.71-4.61 (m, 1H), 3.93-3.79 (m, 2H), 3.30-3.20 (m, 2H), 2.44 (s, 3H), 1.89-1.80 (m, 1H), 1.77 (br d, J = 13.5 Hz, 1H), 1.44-1.28 (m, 3H) | Peak 1/SFC Chiralpak AD-3, 50 × 4.6 mm I.D., 3 μm Mobile phase: 0-95% EtOH (0.1% IPAm, v/v) in CO₂ using a flow rate of 3.4 mL/min |
| | (R)-(6-(6-methyl-1H-indazol-5- | | |
| | yl)thieno[2,3-b]pyridin-2- | | |
| | yl)(tetrahydro-2H-pyran-4- | | |
| | yl)methanol | | |
| B38S | 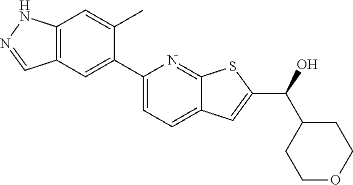 [SMILES: Cc1cc2[nH]ncc2cc1-c1ccc2cc([C@@H](O)C3CCOCC3)sc2n1] | 380.0; ¹H NMR (400 MHz, DMSO- d₆) δ 13.01 (s, 1H), 8.21 (d, J = 8.0 Hz, 1H), 8.07 (s, 1H), 7.79 (s, 1H), 7.54 (d, J = 8.4 Hz, 1H), 7.45 (s, 1H), 7.28 (s, 1H), 5.93 (d, J = 4.8 Hz, 1H), 4.66 (t, J = 5.2 Hz, 1H), 3.90-3.80 (m, 2H), 3.29-3.20 (m, 2H), 2.44 (s, 3H), 1.90-1.72 (m, 2H), 1.42-1.30 (m, 3H) | Peak 2/SFC Chiralpak AD-3, 50 × 4.6 mm I.D., 3 μm Mobile phase: 0-95% EtOH (0.1% IPAm, v/v) in CO₂ using a flow rate of 3.4 mL/min |
| | (S)-(6-(6-methyl-1H-indazol-5- | | |
| | yl)thieno[2,3-b]pyridin-2- | | |
| | yl)(tetrahydro-2H-pyran-4- | | |
| | yl)methanol | | |
| B39R | 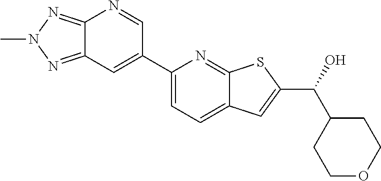 [SMILES: Cn1nc2cc(-c3ccc4cc([C@H](O)C5CCOCC5)sc4n3)cnc2n1] | 382.0; ¹H NMR (400 MHz, DMSO- d₆) δ 9.60 (d, J = 2.0 Hz, 1H), 9.08 (d, J = 2.0 Hz, 1H), 8.31 (d, J = 9.6 Hz, 1H), 8.21 (d, J = 8.4 Hz, 1H), 7.31 (s, 1H), 6.01 (s, 1H), 4.69- 4.67 (m, 1H), 4.59 (s, 3H), 3.93- 3.78 (m, 2H), 3.31-3.17 (m, 2H), 1.9-1.78 (m, 1H), 1.76-1.73 (m, 1H), 1.45-1.27 (m, 3H) | Peak 1/SFC Column: Chiralpak IH-3, 100 × 4.6 mm, I.D., 3 μm Mobile phase: 60% CO₂: 40% EtOH (0.1% IPAm) using a flow rate of 4 mL/min |
| | (R)-(6-(2-methyl-2H- | | |
| | [1,2,3]triazolo[4,5-b]pyridin-6- | | |
| | yl)thieno[2,3-b]pyridin-2- | | |
| | yl)(tetrahydro-2H-pyran-4- | | |
| | yl)methanol | | |
| B39S | 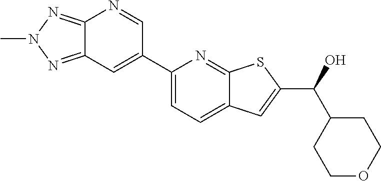 [SMILES: Cn1nc2cc(-c3ccc4cc([C@@H](O)C5CCOCC5)sc4n3)cnc2n1] | 382.0; ¹H NMR (400 MHz, DMSO- d₆) δ 9.60 (d, J = 2.0 Hz, 1H), 9.08 (d, J = 2.0 Hz, 1H), 8.32 (d, J = 9.6 Hz, 1H), 8.22 (d, J = 8.4 Hz, 1H), 7.31 (s, 1H), 6.00 (d, J = 4.4 Hz, 1H), 4.74-4.6 (m, 1H), 4.59 (s, 3H), 3.9-3.72 (m, 2H), 3.29-3.17 (m, 2H), 1.89-1.78 (m, 1H), 1.76- 1.7 (m, 1H), 1.43-1.27 (m, 3H) | Peak 2/SFC Column: Chiralpak IH-3, 100 × 4.6 mm, I.D., 3 μm Mobile phase: 60% CO₂: 40% EtOH (0.1% IPAm) using a flow rate of 4 mL/min |
| | (S)-(6-(2-methyl-2H- | | |
| | [1,2,3]triazolo[4,5-b]pyridin-6- | | |
| | yl)thieno[2,3-b]pyridin-2- | | |
| | yl)(tetrahydro-2H-pyran-4- | | |
| | yl)methanol | | |
| B40S | 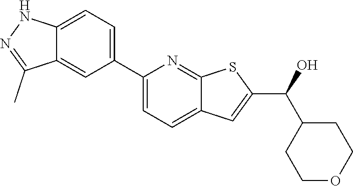 [SMILES: Cc1n[nH]c2ccc(-c3ccc4cc([C@@H](O)C5CCOCC5)sc4n3)cc12] | 380.0; ¹H NMR (400 MHz, DMSO- d₆) δ 12.74 (s, 1H), 8.48 (s, 1H), 8.21-8.17 (m, 2H) 8, 8.05 (d, J = 8.8 Hz, 1H), 7.55 (d, J = 8.8 Hz, 1H), 7.25 (s, 1H), 5.94 (d, J = 4.4 Hz, 1H), 4.68-4.62 (m, 1H), 3.91-3.80 (m, 2H), 3.32-3.21 (m, 2H), 2.57 (s, 3H), 1.87-1.70 (m, 2H), 1.40-1.27 (m, 3H) | Peak 1/SFC Chiralpak IH-3, 100 × 4.6 mm I.D., 3 μm, Mobile phase: 60% CO₂ : 40% EtOH (0.1% IPAm, v/v) using a flow rate of 4 mL/min |
| | (S)-(6-(3-methyl-1H-indazol-5- | | |
| | yl)thieno[2,3-b]pyridin-2- | | |
| | yl)(tetrahydro-2H-pyran-4- | | |
| | yl)methanol | | |
| B40R | 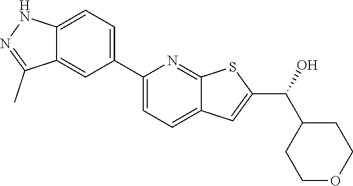 [SMILES: Cc1n[nH]c2ccc(-c3ccc4cc([C@H](O)C5CCOCC5)sc4n3)cc12] | 380.0; ¹H NMR (400 MHz, DMSO- d₆) δ 12.75 (s, 1H), 8.48 (s, 1H), 8.21-8.17 (m, 2H), 8.06 (d, J = 8.4 Hz, 1H), 7.55 (d, J = 8.8 Hz, 1H), 7.25 (s, 1H), 5.94 (d, J = 4.4Hz, 1H), 4.66-4.63 (m, 1H), 3.87- 3.81 (m, 2H), 3.33-3.23 (m, 2H), 2.57 (s, 3H), 1.78-1.75 (m, 2H), 1.38-1.31 (m, 3H) | Peak 2/SFC Chiralpak IH-3, 100 × 4.6 mm I.D., 3 μm, Mobile phase: 60% CO₂:₄0% EtOH (0.1% IPAm, v/v) using a flow rate of 4 mL/min |
| | (R)-(6-(3-methyl-1H-indazol-5- | | |
| | yl)thieno[2,3-b]pyridin-2- | | |
| | yl)(tetrahydro-2H-pyran-4- | | |
| | yl)methanol | | |
| B42 | 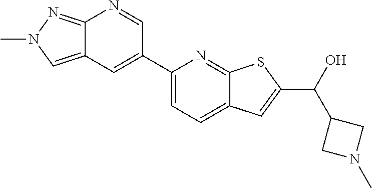 | 366.1; ¹H NMR (400 MHz, DMSO- d₆) δ 9.45 (s, 1H), 9.13 (s, 1H), 8.69 (s, 1H), 8.31 (d, J = 7.6 Hz, 1H), 8.14 (d, J = 8.4 Hz, 1H), 7.34 (s, 1H), 5.30-5.15 (m, 1H), 4.27- 4.11 (m, 7H), 3.18-3.16 (m, 1H), 2.80 (s, 3H) | |
| | (1-methyl-3-azetidinyl)(6-(2- | | |
| | methyl-2H-pyrazolo[3,4- | | |
| | b]pyridin-5-yl)thieno[2,3- | | |
| | b]pyridin-2-yl)methanol | | |
| B43R | 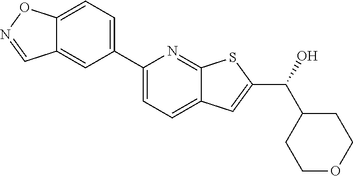 [SMILES: O[C@@H](c1cc2ccc(-c3ccc4oncc4c3)nc2s1)C1CCOCC1] | 367.1; ¹H NMR (400 MHz, DMSO- d₆) δ 8.32 (s, 1H), 8.26 (d, J = 8.4 Hz, 1H), 8.19-8.16 (m, 1H), 7.93 (d, J = 8.4 Hz, 1H), 7.22 (s, 1H), 7.06 (d, J = 8.8 Hz, 1H), 5.92 (s, 1H), 4.63 (d, J = 6.4 Hz, 1H), 3.89- 3.85 (m, 1H), 3.83-3.79 (m, 1H), 3.23-3.19 (m, 2H), 1.82-1.73 (m, 2H), 1.37-1.30 (m, 3H) | Peak 1/SFC Chiralpak AD-3, 150 × 4.6 mm I.D., 3 μm, mobile phase: 50% CO₂ 50% EtOH (0.1% IPAm) using a flow rate of 2.5 mL/min |
| | (R)-(6-(1,2-benzoxazol-5- | | |
| | yl)thieno[2,3-b]pyridin-2- | | |
| | yl)(tetrahydro-2H-pyran-4- | | |
| | yl)methanol | | |
| B43S | 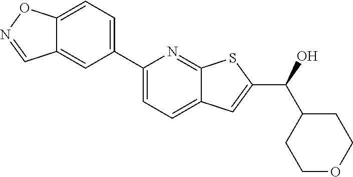 [SMILES: O[C@H](c1cc2ccc(-c3ccc4oncc4c3)nc2s1)C1CCOCC1] | 367.1; ¹H NMR (400 MHz, DMSO- d₆) δ 8.33 (s, 1H), 8.27 (d, J = 8.8 Hz, 1H), 8.19-8.16 (m, 1H), 7.94 (d, J = 8.4 Hz, 1H), 7.23 (s, 1H), 7.07 (d, J = 8.4 Hz, 1H), 5.93 (s, 1H), 4.63 (d, J = 6.4 Hz, 1H), 3.90- 3.81 (m, 2H), 3.24-3.21 (m, 2H), 1.83-1.74 (m, 2H), 1.38-1.31 (m, 3H) | Peak 2/SFC Chiralpak AD-3, 150 × 4.6 mm I.D., 3 μm, mobile phase: 50% CO₂ 50% EtOH (0.1% IPAm) using a flow rate of 2.5 mL/min |
| | (S)-(6-(1,2-benzoxazol-5- | | |
| | yl)thieno[2,3-b]pyridin-2- | | |
| | yl)(tetrahydro-2H-pyran-4- | | |
| | yl)methanol | | |
| B44S | 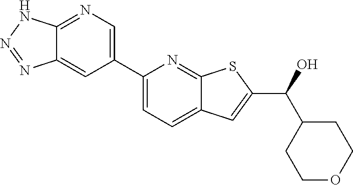 [SMILES: O[C@H](c1cc2ccc(-c3cnc4[nH]nnc4c3)nc2s1)C1CCOCC1] | 368.0; ¹H NMR (400 MHz, DMSO- d₆) δ 9.40 (d, J = 1.6 Hz, 1H), 8.97 (d, J = 2.0 Hz, 1H), 8.29 (d, J = 8.8 Hz, 1H), 8.18 (d, J = 8.4 Hz, 1H), 7.29 (s, 1H), 6.00-5.95 (m, 1H), 4.67 (d, J = 6.8 Hz, 1H), 3.90-3.80 (m, 2H), 3.30-3.24 (m, 2H), 1.84- 1.74 (m, 2H), 1.39-1.34 (m, 3H) | Alternative condition (3) was used Peak 1/SFC Chiralpak AD-3, 50 × 4.6 mm I.D., 3 μm Mobile phase: 50% CO₂ 50% EtOH (0.1% IPAm) using a flow rate of 4 mL/min |
| | (S)-tetrahydro-2H-pyran-4-yl(6- | | |
| | (3H-[1,2,3]triazolo[4,5- | | |
| | b]pyridin-6-yl)thieno[2,3- | | |
| | b]pyridin-2-yl)methanol | | |
| B44R | 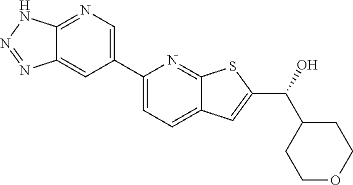 [SMILES: O[C@@H](c1cc2ccc(-c3cnc4[nH]nnc4c3)nc2s1)C1CCOCC1] | 368.0; ¹H NMR (400 MHz, DMSO- d₆) δ 9.40 (d, J = 1.9 Hz, 1 H), 8.97 (d, J = 1.9 Hz, 1 H), 8.29 (d, J = 8.5 Hz, 1 H), 8.18 (d, J = 8.4 Hz, 1 H), 7.30 (s, 1 H), 6.08-5.87 (m, 1 H), 4.67 (br d, J = 6.6 Hz, 1 H), 3.92-3.79 (m, 2 H), 3.26 (br d, J = 11.51 Hz, 2 H), 1.91-1.80 (m, 1 H), 1.76 (br d, J = 13.01 Hz, 1 H), 1.44-1.26 (m, 3 H) | Alternative condition (3) was used Peak 2/SFC Chiralpak AD-3, 50 × 4.6 mm I.D., 3 μm Mobile phase: A: CO₂ B: IPA(0.1% IPAm, v/v) Gradient: A:B = 50:50 Flow rate: 4 mL/min |
| | (R)-tetrahydro-2H-pyran-4-yl(6- | | |
| | (3H-[1,2,3]triazolo[4,5- | | |
| | b]pyridin-6-yl)thieno[2,3- | | |
| | b]pyridin-2-yl)methanol | | |
| B45 | 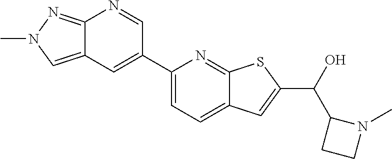 | 366.1; ¹H NMR (400 MHz, DMSO- d₆) δ 9.37 (d, J = 1.6 Hz, 1H), 8.90 (d, J = 2.4 Hz, 1H), 8.53 (s, 1H), 8.25 (d, J = 8.8 Hz, 1H), 8.07 (d, J = 8.4 Hz, 1H), 7.31 (s, 1H), 5.96 (d, J = 4.8 Hz, 1H), 4.85-4.82 (m, 1H), 4.23 (s, 3H), 3.26-3.10 (m, 2H), 2.67-2.65 (m, 1H), 2.30 (s, 3H), 1.91-1.79 (m, 2H) | |
| | 1-methyl-2-azetidinyl)(6-(2- | | |
| | methyl-2H-pyrazolo[3,4- | | |
| | b]pyridin-5-yl)thieno[2,3- | | |
| | b]pyridin-2-yl)methanol | | |
| B46S | 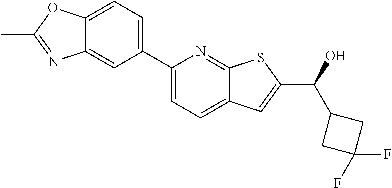 [SMILES: Cc1nc2cc(-c3ccc4cc([C@@H](O)C5CC(F)(F)C5)sc4n3)ccc2o1] | 387.0; ¹H NMR (400 MHz, DMSO- d₆) δ 8.39 (d, J = 1.6 Hz, 1H), 8.24- 8.17 (m, 2H), 8.07 (d, J = 8.4 Hz, 1H), 7.77 (d, J = 8.8 Hz, 1H), 7.32 (s, 1H), 6.25 (d, J = 5.2 Hz, 1H), 4.93-4.91 (m, 1H), 2.65 (s, 3H), 2.61-2.54 (m, 5H) | Peak 2/SFC Chiralpak IH- 3,100 × 4.6 mm I.D., 3 μm Mobile phase: 50% CO₂: 50% EtOH(0.1% IPAm, v/v) using a flow rate of 3.4 mL/min |
| | (S)-(3,3-difluorocyclobutyl)(6- | | |
| | (2-methyl-1,3-benzoxazol-5- | | |
| | yl)thieno[2,3-b]pyridin-2- | | |
| | yl)methanol | | |
| B46R | 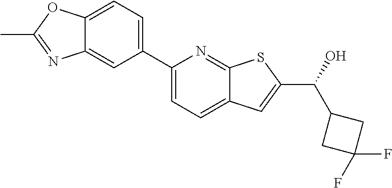 [SMILES: Cc1nc2cc(-c3ccc4cc([C@H](O)C5CC(F)(F)C5)sc4n3)ccc2o1] | 387.0; ¹H NMR (400 MHz, DMSO- d₆) δ 8.38 (d, J = 1.6 Hz, 1H), 8.24- 8.16 (m, 2H), 8.06 (d, J = 8.4 Hz, 1H), 7.76 (d, J = 8.4 Hz, 1H), 7.32 (s, 1H), 6.26 (d, J = 4.8 Hz, 1H), 4.92 (s, 1H), 2.65 (s, 3H), 2.65- 2.54 (m, 5H) | Peak 1/SFC Chiralpak IH- 3,100 × 4.6 mm I.D., 3 μm Mobile phase: 50% CO₂: 50% EtOH(0.1% IPAm, v/v) using a flow rate of 3.4 mL/min |
| | (R)-(3,3-difluorocyclobutyl)(6- | | |
| | (2-methyl-1,3-benzoxazol-5- | | |
| | yl)thieno[2,3-b]pyridin-2- | | |
| | yl)methanol | | |
| B47S | 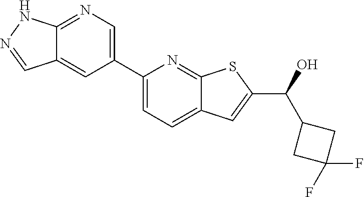 [SMILES: O[C@H](c1cc2ccc(-c3cnc4[nH]ncc4c3)nc2s1)C1CC(F)(F)C1] | 373.0; ¹H NMR (400 MHz, DMSO- d₆) δ 13.80 (br d, J = 1.9 Hz, 1H), 9.31 (d, J = 2.1 Hz, 1H), 8.95 (d, J = 2.0 Hz, 1H), 8.31-8.23 (m, 2H), 8.11 (d, J = 8.5 Hz, 1H), 7.34 (s, 1H), 6.27 (br d, J = 1.2 Hz, 1H), 4.93 (br s, 1H), 2.63-2.53 (m, 5H) | Peak 1/SFC Chiralpak IG-3, 50 × 4.6 mm I.D., 3 μm, Mobile phase: 50% CO₂: 50% IPA(0.1% IPAm, v/v) using a flow rate of 4 mL/min |
| | (S)-(3,3-difluorocyclobutyl)(6- | | |
| | (1H-pyrazolo[3,4-b]pyridin-5- | | |
| | yl)thieno[2,3-b]pyridin-2- | | |
| | yl)methanol | | |
| B47R | 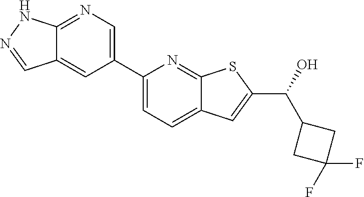 [SMILES: O[C@@H](c1cc2ccc(-c3cnc4[nH]ncc4c3)nc2s1)C1CC(F)(F)C1] | 373.0; ¹H NMR (400 MHz, DMSO- d₆) δ 13.80 (br d, J = 1.9 Hz, 1H), 9.31 (d, J = 2.1 Hz, 1H), 8.95 (d, J = 2.0 Hz, 1H), 8.31-8.23 (m, 2H), 8.11 (d, J = 8.5 Hz, 1H), 7.34 (s, 1H), 6.27 (br d, J = 1.2 Hz, 1H), 4.93 (br s, 1H), 2.63-2.53 (m, 5H) | Peak 2/SFC Chiralpak IG-3, 50 × 4.6mm I.D., 3 μm, Mobile phase: 50% CO₂: 50% IPA(0.1% IPAm, v/v) using a flow rate of 4 mL/min |
| | (R)-(3,3-difluorocyclobutyl)(6- | | |
| | (1H-pyrazolo[3,4-b]pyridin-5- | | |
| | yl)thieno[2,3-b]pyridin-2- | | |
| | yl)methanol | | |
| B50S | 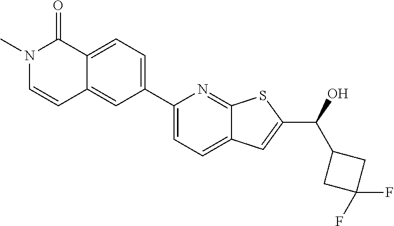 [SMILES: Cn1ccc2cc(-c3ccc4cc([C@@H](O)C5CC(F)(F)C5)sc4n3)ccc2c1=O] | 413.0; ¹H NMR (400 MHz, DMSO- d₆) δ 8.44 (s, 1H), 8.33-8.25 (m, 3H), 8.13 (d, J = 8.4 Hz, 1H), 7.52 (d, J = 7.6 Hz, 1H), 7.36 (s, 1H), 6.76 (d, J = 7.2 Hz, 1H), 6.29 (s, 1H), 4.94 (d, J = 4.0 Hz, 1H), 3.53 (s, 3H), 2.60-2.56 (m, 5H) | Peak 1/SFC Chiralpak IH-3, 100 × 4.6 mm I.D., 3 μm, Mobile phase: 50% CO₂: 50% EtOH (0.1% IPAm, v/v) using a flow rate of 4 mL/min |
| | 6-(2-((S)-(3,3- | | |
| | difluorocyclobutyl)(hydroxy)met | | |
| | hyl)thieno[2,3-b]pyridin-6-yl)-2- | | |
| | methyl-1(2H)-isoquinolinone | | |
| B50R | 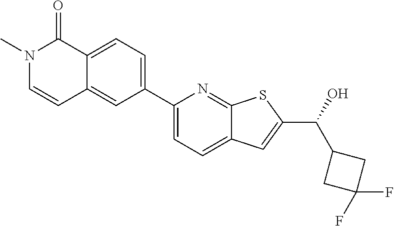 [SMILES: Cn1ccc2cc(-c3ccc4cc([C@H](O)C5CC(F)(F)C5)sc4n3)ccc2c1=O] | 413.0; ¹H NMR (400 MHz, DMSO- d₆) δ 8.44 (s, 1 H), 8.36-8.22 (m, 3H), 8.13 (d, J = 8.4 Hz, 1H), 7.52 (d, J = 7.3 Hz, 1H), 7.36 (s, 1H), 6.77 (d, J = 7.25 Hz, 1H), 6.29 (br s, 1H), 4.94 (br d, J = 3.5 Hz, 1H), 3.53 (s, 3H), 2.69-2.53 (m, 5H) | Peak 2/SFC Chiralpak IH-3, 100 × 4.6 mm I.D., 3 μm, Mobile phase: 50% CO₂: 50% EtOH (0.1% IPAm, v/v) using a flow rate of 4 mL/min |
| | 6-(2-((R)-(3,3- | | |
| | difluorocyclobutyl)(hydroxy)met | | |
| | hyl)thieno[2,3-b]pyridin-6-yl)-2- | | |
| | methyl-1(2H)-isoquinolinone | | |
| B51S | 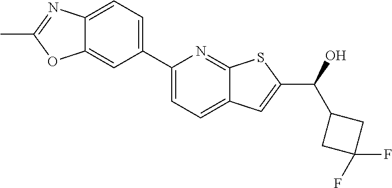 [SMILES: Cc1nc2ccc(-c3ccc4cc([C@@H](O)C5CC(F)(F)C5)sc4n3)cc2o1] | 387.0; ¹H NMR (400 MHz, DMSO- d6) § 8.40 (s, 1H), 8.24 (d, J = 8.8 Hz, 1H), 8.17 (dd, J = 1.6 Hz, 6.4 Hz, 1H), 8.07 (d, J = 8.4 Hz, 1H), 7.75 (d, J = 8.0 Hz, 1H), 7.33 (d, J = 0.8 Hz, 1H), 6.26 (s, 1H), 4.93 (d, J = 4.0 Hz, 1H), 2.66 (s, 3H), 2.63- 2.55 (m, 5H) | Peak 1/SFC Chiralpak IG-3, 50 × 4.6 mm I.D., 3 μm, Mobile phase: 50% CO₂ 50% EtOH (0.1% IPAm, v/v) using a flow rate of 4 mL/min |
| | (S)-(3,3-difluorocyclobutyl)(6- | | |
| | (2-methyl-1,3-benzoxazol-6- | | |
| | yl)thieno[2,3-b]pyridin-2- | | |
| | yl)methanol | | |
| B51R | 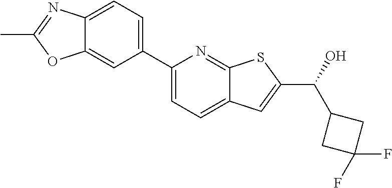 [SMILES: Cc1nc2ccc(-c3ccc4cc([C@H](O)C5CC(F)(F)C5)sc4n3)cc2o1] | 387.0; ¹H NMR (400 MHz, DMSO- d₆) δ 8.40 (d, J = 1.3 Hz, 1H), 8.24 (d, J = 8.5 Hz, 1H), 8.17 (dd, J = 8.4, 1.6 Hz, 1H), 8.07 (d, J = 8.5 Hz, 1H), 7.76 (d, J = 8.5 Hz, 1H), 7.33 (d, J = 0.8 Hz, 1H), 6.26 (s, 1H), 4.93 (br d, J = 3.9 Hz, 1H), 2.66 (s, 3H), 2.64-2.51 (m, 5H) | Peak 2/SFC Chiralpak IG-3, 50 × 4.6 mm I.D., 3 μm, Mobile phase: 50% CO₂ 50% EtOH (0.1% IPAm, v/v) using a flow rate of 4 mL/min |
| | (R)-(3,3-difluorocyclobutyl)(6- | | |
| | (2-methyl-1,3-benzoxazol-6- | | |
| | yl)thieno[2,3-b]pyridin-2- | | |
| | yl)methanol | | |
| B52S | 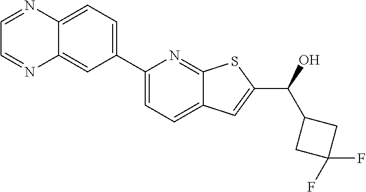 [SMILES: O[C@H](c1cc2ccc(-c3ccc4nccnc4c3)nc2s1)C1CC(F)(F)C1] | 384.0; ¹H NMR (400 MHz, DMSO- d₆) δ 9.01 (d, J = 1.6 Hz, 1H) 8.97 (d, J = 1.6 Hz, 1H), 8.82 (d, J = 2.0 Hz, 1H), 8.70 (dd, J = 8.8 Hz, 2.0 Hz, 1H), 8.34-8.30 (m, 2H), 8.22 (d, J = 8.8 Hz, 1H), 7.38 (s, 1H), 6.30 (d, J = 4.8 Hz, 1H), 4.95 (t, J = 8.4 Hz, 1H), 2.61-2.56 (m, 5H) | Peak 1/SFC: Chiralpak IH- 3,100 × 4.6 mm I.D., 3 μm Mobile phase: 50% CO₂ 50% EtOH (0.1% IPAm, v/v) using a flow rate of 3.4 mL/min |
| | (S)-(3,3-difluorocyclobutyl)(6- | | |
| | (6-quinoxalinyl)thieno[2,3- | | |
| | b]pyridin-2-yl)methanol | | |
| B52R | 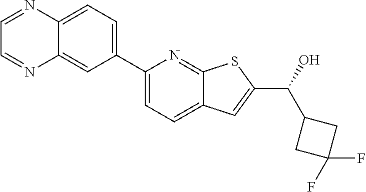 [SMILES: O[C@@H](c1cc2ccc(-c3ccc4nccnc4c3)nc2s1)C1CC(F)(F)C1] | 384.0; ¹H NMR (400 MHz, DMSO- d₆) δ 9.01 (d, J = 1.8 Hz, 1H), 8.98 (d, J = 1.8 Hz, 1H), 8.82 (d, J = 1.9 Hz, 1H), 8.70 (dd, J = 8.8, 2.0 Hz, 1H), 8.36-8.27 (m, 2H), 8.22 (d, J = 8.8 Hz, 1H), 7.38 (d, J = 0.6Hz, 1H), 6.30 (d, J = 5.1 Hz, 1H), 4.95 (t, J = 4.5 Hz, 1H), 2.69-2.54 (m, 5H) | Peak 2/SFC: Chiralpak IH- 3,100 × 4.6 mm I.D., 3 μm Mobile phase: 50% CO₂ 50% EtOH (0.1% IPAm, v/v) using a flow rate of 3.4 mL/min |
| | (R)-(3,3-difluorocyclobutyl)(6- | | |
| | (6-quinoxalinyl)thieno[2,3- | | |
| | b]pyridin-2-yl)methanol | | |
| B53S | 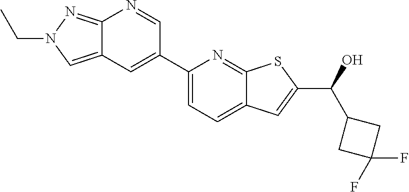 [SMILES: CCn1cc2cc(-c3ccc4cc([C@@H](O)C5CC(F)(F)C5)sc4n3)cnc2n1] | 401.1; ¹H NMR (400 MHz, DMSO- d₆) δ 9.38 (d, J = 2.3 Hz, 1H), 8.90 (d, J = 2.3 Hz, 1H), 8.58 (s, 1H), 8.26 (d, J = 8.4 Hz, 1H), 8.09 (d, J = 8.4 Hz, 1H), 7.33 (s, 1H), 6.26 (br s, 1H), 4.93 (br d, J = 4.5 Hz, 1H), 4.52 (q, J = 7.3 Hz, 2H), 2.65- 2.54 (m, 5H), 1.55 (t, J = 7.3 Hz, 3H) | Alternative condition (1) was used Peak 1/SFC: Chiralpak AD-3, 50 × 4.6 mm I.D., 3 μm, mobile phase: 50% CO₂ 50% IPA (0.1% IPAm, v/v) using a flow rate of 3.4 mL/min |
| | (S)-(3,3-difluorocyclobutyl)(6- | | |
| | (2-ethyl-2H-pyrazolo[3,4- | | |
| | b]pyridin-5-yl)thieno[2,3- | | |
| | b]pyridin-2-yl)methanol | | |
| B53R | 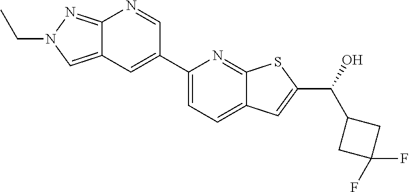 [SMILES: CCn1cc2cc(-c3ccc4cc([C@H](O)C5CC(F)(F)C5)sc4n3)cnc2n1] | 401.0; ¹H NMR (400 MHz, DMSO- d₆) δ 9.38 (d, J = 2.3 Hz, 1H), 8.90 (d, J = 2.4 Hz, 1H), 8.58 (s, 1H), 8.26 (d, J = 8.4 Hz, 1H), 8.09 (d, J = 8.4 Hz, 1H), 7.33 (d, J = 0.6 Hz, 1H), 6.26 (d, J = 5.0 Hz, 1H), 4.93 (t, J = 4.6 Hz, 1H), 4.52 (q, J = 7.3 Hz, 2H), 2.64-2.54 (m, 5H), 1.55 (t, J = 7.3 Hz, 3H) | Alternative condition (1) was used Peak 2/SFC: Chiralpak AD-3, 50 × 4.6 mm I.D., 3 μm, mobile phase: 50% CO₂ 50% IPA (0.1% IPAm, v/v) using a flow rate of 3.4 mL/min |
| | (R)-(3,3-difluorocyclobutyl)(6- | | |
| | (2-ethyl-2H-pyrazolo[3,4- | | |
| | b]pyridin-5-yl)thieno[2,3- | | |
| | b]pyridin-2-yl)methanol | | |
| B55S | 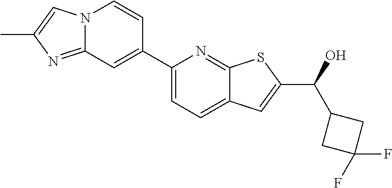 [SMILES: Cc1cn2ccc(-c3ccc4cc([C@@H](O)C5CC(F)(F)C5)sc4n3)cc2n1] | 386.0; ¹H NMR (400 MHz, DMSO- d₆) δ 8.54 (d, J = 7.1 Hz, 1H), 8.26- 8.22 (m, 2H), 8.14 (d, J = 8.4 Hz, 1H), 7.76 (s, 1H), 7.72-7.67 (m, 1H), 7.33 (s, 1H), 6.27 (d, J = 5.1 Hz, 1H), 4.93 (t, J = 4.6 Hz, 1H), 2.59-2.54 (m, 5H), 2.37 (s, 3H) | Alternative condition (2) was used Peak 1/SFC Chiralpak IG-3, 100 × 4.6 mm, 3 μm; Mobile phase: 50% CO₂ 50% EtOH (0.1% IPAm, v/v) using a flow rate of 3 mL/min |
| | (S)-(3,3-difluorocyclobutyl)(6- | | |
| | (2-methylimidazo[1,2-a]pyridin- | | |
| | 7-yl)thieno[2,3-b]pyridin-2- | | |
| | yl)methanol | | |
| B55R | 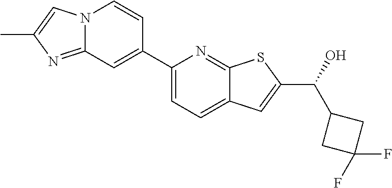 [SMILES: Cc1cn2ccc(-c3ccc4cc([C@H](O)C5CC(F)(F)C5)sc4n3)cc2n1] | 386.0; ¹H NMR (400 MHz, DMSO- d₆) δ 8.55 (d, J = 7.1 Hz, 1H), 8.27- 8.21 (m, 2H), 8.14 (d, J = 8.5 Hz, 1H), 7.76 (s, 1H), 7.70 (dd, J = 7.1, 1.6 Hz, 1H), 7.34 (s, 1H), 6.27 (d, J = 5.0 Hz, 1H), 4.93 (t, J = 4.6 Hz, 1H), 2.65-2.52 (m, 5H), 2.37 (s, 3H) | Alternative condition (2) was used Peak 2/SFC Chiralpak IG-3, 100 × 4.6 mm, 3 μm; Mobile phase: 50% CO₂ 50% EtOH (0.1% IPAm, v/v) using a flow rate of 3 mL/min |
| | (R)-(3,3-difluorocyclobutyl)(6- | | |
| | (2-methylimidazo[1,2-a]pyridin- | | |
| | 7-yl)thieno[2,3-b]pyridin-2- | | |
| | yl)methanol | | |
| B56 | 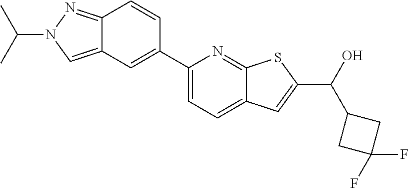 [SMILES: CC(C)n1cc2cc(-c3ccc4cc(C(O)C5CC(F)(F)C5)sc4n3)ccc2n1] | 414.0; ¹H NMR (400 MHz, DMSO- d₆) δ 8.53 (s, 1H), 8.49 (s, 1H), 8.20 (d, J = 8.4 Hz, 1H), 8.07 (dd, J = 1.7, 9.1 Hz, 1H), 8.01 (d, J = 8.5 Hz, 1H), 7.71 (d, J = 9.1 Hz, 1H), 7.31 (s, 1H), 6.22 (d, J = 5.0 Hz, 1H), 4.91-4.83 (m, 2H), 2.61- 2.53 (m, 5H), 1.58 (d, J = 6.6 Hz, 6H) | |
| | (3,3-difluorocyclobutyl)(6-(2-(2- | | |
| | propanyl)-2H-indazol-5- | | |
| | yl)thieno[2,3-b]pyridin-2- | | |
| | yl)methanol | | |
| B56S | 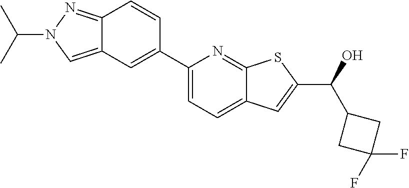 [SMILES: CC(C)n1cc2cc(-c3ccc4cc([C@@H](O)C5CC(F)(F)C5)sc4n3)ccc2n1] | 414.1; ¹H NMR (400 MHz, DMSO- d₆) δ 8.52 (s, 1H), 8.49 (s, 1H), 8.20 (d, J = 8.4 Hz, 1H), 8.08 (dd, J = 1.7, 9.1 Hz, 1H), 8.03 (d, J = 8.5 Hz, 1H), 7.71 (d, J = 9.1 Hz, 1H), 7.30 (s, 1H), 6.23 (d, J = 5.0 Hz, 1H), 4.91-4.84 (m, 2H), 2.61- 2.50 (m, 5H), 1.58 (d, J = 6.6 Hz, 6H) | Peak 1/SFC: Chiralpak IH- 3(50 × 4.6 mm, 3 μm); Mobile phase: 0-50% EtOH (0.1% IPAm, v/v) in CO₂ using a flow rate of 3 mL/min |
| | (S)-(3,3-difluorocyclobutyl)(6- | | |
| | (2-(2-propanyl)-2H-indazol-5- | | |
| | yl)thieno[2,3-b]pyridin-2- | | |
| | yl)methanol | | |
| B56R | 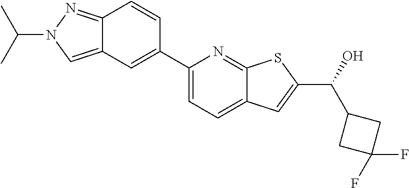 [SMILES: CC(C)n1cc2cc(-c3ccc4cc([C@H](O)C5CC(F)(F)C5)sc4n3)ccc2n1] | 414.1; ¹H NMR (400 MHz, DMSO- d₆) δ 8.55-8.50 (m, 1H), 8.49 (s, 1H), 8.20 (d, J = 8.4 Hz, 1H), 8.07 (dd, J = 1.6, 9.1 Hz, 1H), 8.01 (d, J = 8.4 Hz, 1H), 7.71 (d, J = 9.1 Hz, 1H), 7.30 (s, 1H), 6.23 (d, J = 5.0 Hz, 1H), 4.99-4.75 (m, 2H), 2.63- 2.53 (m, 5H), 1.58 (d, J = 6.6 Hz, 6H) | Peak 2/SFC: Chiralpak IH- 3(50 × 4.6 mm, 3 μm); Mobile phase: 0-50% EtOH (0.1% IPAm, v/v) in CO₂ using a flow rate of 3 mL/min |
| | (R)-(3,3-difluorocyclobutyl)(6- | | |
| | (2-(2-propanyl)-2H-indazol-5- | | |
| | yl)thieno[2,3-b]pyridin-2- | | |
| | yl)methanol | | |
| B57S | 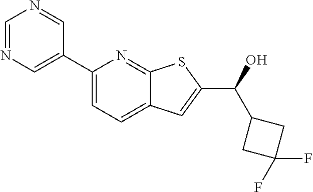 [SMILES: O[C@H](c1cc2ccc(-c3cncnc3)nc2s1)C1CC(F)(F)C1] | 334.0; ¹H NMR (400 MHz, DMSO- d₆) δ 9.50 (s, 2H), 9.26 (s, 1H), 8.33 (d, J = 8.4 Hz, 1H), 8.15 (d, J = 8.4 Hz, 1H), 7.37 (s, 1H), 6.30 (s, 1H), 4.95 (d, J = 4.0 Hz, 1H), 2.59- 2.55 (m, 5H) | Peak 1/SFC Chiralpak AD-3, 50 × 4.6 mm I.D., 3 μm Mobile phase: 50% CO₂: 50% EtOH(0.1% IPAm, v/v) using a flow rate of 4 mL/min |
| | (S)-(3,3-difluorocyclobutyl)(6- | | |
| | (5-pyrimidinyl)thieno[2,3- | | |
| | b]pyridin-2-yl)methanol | | |
| B57R | 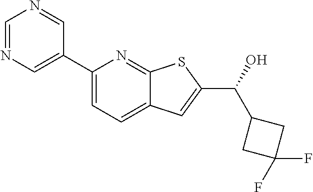 [SMILES: O[C@@H](c1cc2ccc(-c3cncnc3)nc2s1)C1CC(F)(F)C1] | 334.0; ¹H NMR (400 MHz, DMSO- d₆) δ 9.51 (s, 2H), 9.25 (s, 1H), 8.33 (d, J = 8.4 Hz, 1H), 8.15 (d, J = 8.4 Hz, 1H), 7.37 (s, 1H), 6.30 (s, 1H), 4.95 (d, J = 4.4 Hz, 1H), 2.71- 2.51 (m, 5H) | Peak 2/SFC Chiralpak AD-3, 50 × 4.6 mm I.D., 3 μm Mobile phase: 50% CO₂: 50% EtOH(0.1% IPAm, v/v) using a flow rate of 4 mL/min |
| | (R)-(3,3-difluorocyclobutyl)(6- | | |
| | (5-pyrimidinyl)thieno[2,3- | | |
| | b]pyridin-2-yl)methanol | | |
| B62 | 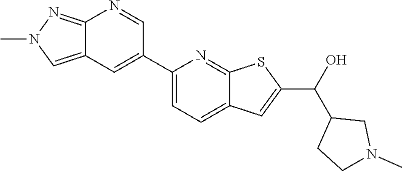 | 380.1; ¹H NMR (400 MHz, DMSO- d₆) δ 9.37 (d, J = 2.4 Hz, 1H), 8.91 (d, J = 2.0 Hz, 1H), 8.54 (s, 1H), 8.25 (d, J = 8.4 Hz, 1H), 8.08 (d, J = 8.4 Hz, 1H), 7.32 (d, J = 7.2 Hz, 1H), 6.01 (s, 1H), 4.75-4.65 (m, 1H), 4.24 (s, 3H), 2.60-2.40 (m, 4H), 2.22 (d, J = 17.6 Hz, 3H), 2.00- 1.79 (m, 1H), 1.78-1.65 (m, 1H), 1.60-1.50 (m, 1H) | |
| | (6-(2-methyl-2H-pyrazolo[3,4- | | |
| | b]pyridin-5-yl)thieno[2,3- | | |
| | b]pyridin-2-yl)(1-methyl-3- | | |
| | pyrrolidinyl)methanol | | |
| B63 | 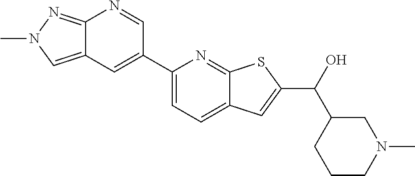 | 394.3; ¹H NMR (400 MHz, DMSO- d₆) δ 9.37 (d, J = 1.6 Hz, 1H), 8.91 (s, 1H), 8.54 (s, 1H), 8.25 (d, J = 8.0 Hz, 1H), 8.08 (d, J = 8.0 Hz, 1H), 7.26 (d, J = 7.6 Hz, 1H), 6.10- 5.85 (m, 1H), 4.75-4.69 (m, 1H), 4.24 (s, 3H), 2.92 (d, J = 6.8 Hz, 1H), 2.64 (d, J = 8.8 Hz, 1H), 2.14 (d, J = 29.6 Hz, 3H), 1.85 (s, 4H), 1.75-1.55 (m, 1H), 1.41 (d, J = 11.2 Hz, 2H) | |
| | 1-methyl-3-piperidinyl)(6-(2- | | |
| | methyl-2H-pyrazolo[3,4- | | |
| | b]pyridin-5-yl)thieno[2,3- | | |
| | b]pyridin-2-yl)methanol | | |
| B64R | 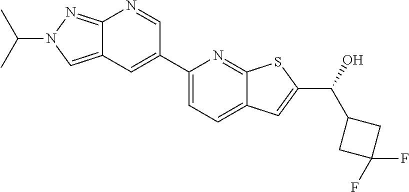 [SMILES: CC(C)n1cc2cc(-c3ccc4cc([C@H](O)C5CC(F)(F)C5)sc4n3)cnc2n1] | 415.0; ¹H NMR (400 MHz, DMSO- d₆) δ 9.38 (d, J = 2.0 Hz, 1H), 8.89 (d, J = 2.4 Hz, 1H), 8.60 (s, 1H), 8.26 (d, J = 8.4 Hz, 1H), 8.09 (d, J = 8.4 Hz, 1H), 7.33 (s, 1H), 6.27 (s, 1H), 5.05-4.80 (m, 2H), 2.65- 2.51 (m, 5H), 1.60 (d, J = 6.8 Hz, 6H) | Peak 1/SFC: Chiralpak AD-3, 50 × 4.6 mm I.D., 3 μm Mobile phase: 50% CO₂: 50% EtOH(0.1% IPAm, v/v) using a flow rate of 2.5 mL/min |
| | (R)-(3,3-difluorocyclobutyl)(6- | | |
| | (2-(2-propanyl)-2H- | | |
| | pyrazolo[3,4-b]pyridin-5- | | |
| | yl)thieno[2,3-b]pyridin-2- | | |
| | yl)methanol | | |
| B64S | 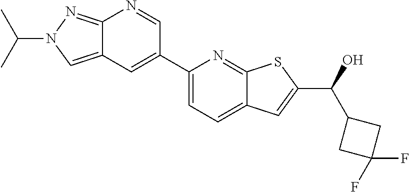 [SMILES: CC(C)n1cc2cc(-c3ccc4cc([C@@H](O)C5CC(F)(F)C5)sc4n3)cnc2n1] | 415.0; ¹H NMR (400 MHz, DMSO- d₆) δ 9.38 (d, J = 2.0 Hz, 1H), 8.89 (d, J = 2.4 Hz, 1H), 8.60 (s, 1H), 8.26 (d, J = 8.4 Hz, 1H), 8.09 (d, J = 8.4 Hz, 1H), 7.33 (s, 1H), 6.27 (s, 1H), 5.05-4.75 (m, 2H), 2.65- 2.51 (m, 5H), 1.60 (d, J = 6.8 Hz, 6H | Peak 2/SFC: Chiralpak AD-3, 50 × 4.6 mm I.D., 3 μm Mobile phase: 50% CO₂: 50% EtOH(0.1% IPAm, v/v) using a flow rate of 2.5 mL/min |
| | (S)-(3,3-difluorocyclobutyl)(6- | | |
| | (2-(2-propanyl)-2H- | | |
| | pyrazolo[3,4-b]pyridin-5- | | |
| | yl)thieno[2,3-b]pyridin-2- | | |
| | yl)methanol | | |
| B65 | 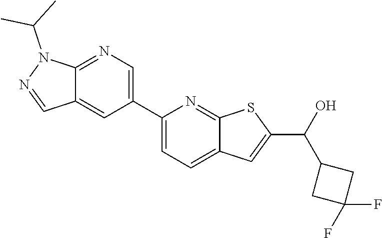 [SMILES: CC(C)n1ncc2cc(-c3ccc4cc(C(O)C5CC(F)(F)C5)sc4n3)cnc21] | 415.0; ¹H NMR (400 MHz, DMSO-d6) 8 9.32 (d, J = 2.0 Hz, 1H), 8.94 (d, J = 2.0 Hz, 1H), 8.29- 8.23 (m, 2H), 8.10 (d, J = 8.4 Hz, 1H), 7.34 (s, 1H), 6.26 (d, J = 5.2 Hz, 1H), 5.23-5.22 (m, 1H), 4.93 (t, J = 4.8 Hz, 1H), 2.61- 2.55 (m, 5H), 1.54 (d, J = 6.8 Hz, 6H) | |
| | (3,3-difluorocyclobutyl)(6-(1-(2- | | |
| | propanyl)-1H-pyrazolo[3,4- | | |
| | b]pyridin-5-yl)thieno[2,3- | | |
| | b]pyridin-2-yl)methanol | | |
| B65R | 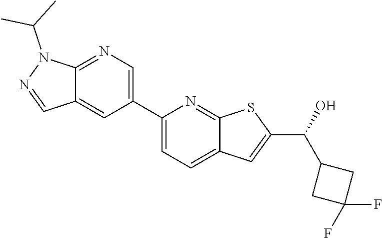 [SMILES: CC(C)n1ncc2cc(-c3ccc4cc([C@H](O)C5CC(F)(F)C5)sc4n3)cnc21] | 415.0; ¹H NMR (400 MHz, DMSO- d₆) δ 9.32 (d, J = 2.0 Hz, 1H), 8.94 (d, J = 2.0 Hz, 1H), 8.29-8.23 (m, 2H), 8.10 (d, J = 8.4 Hz, 1H), 7.34 (s, 1H), 6.26 (d, J = 3.6 Hz, 1H), 5.30-5.20 (m, 1H), 4.93 (br s, 1H), 2.75-2.51 (m, 5H), 1.53 (d, J = 6.8 Hz, 6H) | Peak 1/SFC: Chiralpak AD-3, 50 × 4.6 mm I.D., 3 μm Mobile phase: 50% CO₂: 50% EtOH(0.1% IPAm, v/v) using a flow rate of 2.5 mL/min |
| | (R)-(3,3-difluorocyclobutyl)(6- | | |
| | (1-(2-propanyl)-1H- | | |
| | pyrazolo[3,4-b]pyridin-5- | | |
| | yl)thieno[2,3-b]pyridin-2- | | |
| | yl)methanol | | |
| B65S | 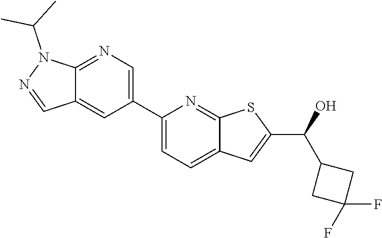 [SMILES: CC(C)n1ncc2cc(-c3ccc4cc([C@@H](O)C5CC(F)(F)C5)sc4n3)cnc21] | 415.0; ¹H NMR (400 MHz, DMSO- d₆) δ 9.32 (d, J = 2.0 Hz, 1H), 8.94 (d, J = 2.0 Hz, 1H), 8.29-8.23 (m, 2H), 8.10 (d, J = 8.4 Hz, 1H), 7.34 (s, 1H), 6.22 (s, 1H), 5.35-5.15 (m, 1H), 4.93 (br s, 1H), 2.75-2.51 (m, 5H), 1.54 (d, J = 6.8 Hz, 6H) | Peak 2/SFC: Chiralpak AD-3, 50 × 4.6 mm I.D., 3 μm Mobile phase: 50% CO₂: 50% EtOH(0.1% IPAm, v/v) using a flow rate of 2.5 mL/min |
| | (S)-(3,3-difluorocyclobutyl)(6- | | |
| | (1-(2-propanyl)-1H- | | |
| | pyrazolo[3,4-b]pyridin-5- | | |
| | yl)thieno[2,3-b]pyridin-2- | | |
| | yl)methanol | | |
| B31S | 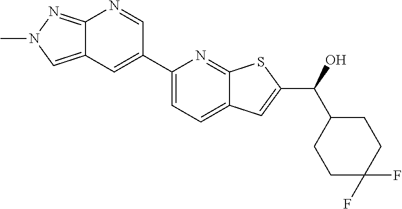 [SMILES: Cn1cc2cc(-c3ccc4cc([C@@H](O)C5CCC(F)(F)CC5)sc4n3)cnc2n1] | 415.2; ¹H NMR (400 MHz, DMSO- d₆) δ 9.38 (d, J = 2.3 Hz, 1H) 8.91 (d, J = 2.3 Hz, 1H) 8.55 (s, 1H) 8.26 (d, J = 8.5 Hz, 1H) 8.09 (d, J = 8.3 Hz, 1H) 7.29 (s, 1H) 6.03 (d, J = 4.6 Hz, 1H) 4.79-4.73 (m, 1H) 4.24 (s, 3H) 2.08-1.89 (m, 3H) 1.86-1.70 (m, 3H) 1.59 (br d, J = 12.9 Hz, 1H) 1.45-1.32 (m, 2H) ¹⁹F NMR (376 MHz, DMSO-d6) 8- 99.87 (d, J = 233.1 Hz, 1F), −89.57 (d, J = 229.4 Hz, 1F) | Peak 2/SFC: Chiralcel OX, 150 × 21 mm I.D., 5 μm, column Mobile phase: 50% CO₂: 50% MeOH(0.2% diethylamine, v/v) using a flow rate of 130 mL/min |
| | (S)-(4,4-difluorocyclohexyl)(6- | | |
| | (2-methyl-2H-pyrazolo[3,4- | | |
| | b]pyridin-5-yl)thieno[2,3- | | |
| | b]pyridin-2-yl)methanol | | |
| B31R | 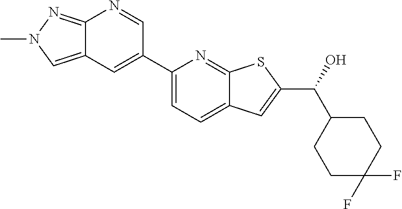 [SMILES: Cn1cc2cc(-c3ccc4cc([C@H](O)C5CCC(F)(F)CC5)sc4n3)cnc2n1] | 415.0; 9.38 (d, J = 2.3 Hz, 1H) 8.91 (d, J = 2.3 Hz, 1H) 8.55 (s, 1H) 8.26 (d, J = 8.5 Hz, 1H) 8.09 (d, J = 8.3 Hz, 1H) 7.29 (s, 1H) 6.03 (d, J = 4.6 Hz, 1H) 4.79-4.73 (m, 1H) 4.24 (s, 3H) 2.08-1.92 (m, 3H) 1.87-1.70 (m, 3H) 1.59 (br d, J = 13.1 Hz, 1H) 1.44-1.32 (m, 2H); ¹⁹F NMR (376 MHz, DMSO-d6) 8 −99.87 (d, J = 233.1 Hz, IF), −89.57 (d, J = 229.4 Hz, 1F). | Peak 1/SFC: Chiralcel OX, 150 × 21 mm I.D., 5 μm, column Mobile phase: 50% CO₂: 50% MeOH(0.2% diethylamine, v/v) using a flow rate of 130 mL/min |
| | (R)-(4,4-difluorocyclohexyl)(6- | | |
| | (2-methyl-2H-pyrazolo[3,4- | | |
| | b]pyridin-5-yl)thieno[2,3- | | |
| | b]pyridin-2-yl)methanol | | |
| B27S | 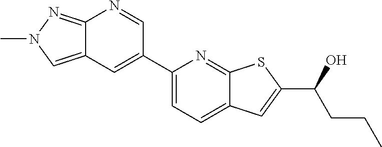 [SMILES: CCC[C@H](O)c1cc2ccc(-c3cnc4nn(C)cc4c3)nc2s1] | 339.2; ¹H NMR (400 MHz, DMSO- d₆) δ 9.38 (d, J = 2.3 Hz, 1H) 8.91 (d, J = 2.3 Hz, 1H) 8.54 (s, 1H) 8.25 (d, J = 8.5 Hz, 1H) 8.08 (d, J = 8.5 Hz, 1H) 7.27 (s, 1H) 5.85 (d, J = 4.8 Hz, 1H) 4.93-4.86 (m, 1H) 4.24 (s, 3H) 1.82-1.69 (m, 2H) 1.49-1.31 (m, 2H) 0.93 (t, J = 7.4 Hz, 3H) | Peak 1/SFC: Chiralpak AS, 250 × 20 mm I.D., 5 μm, column Mobile phase: 70% CO₂: 30% MeOH(0.2% diethylamine, v/v) using a flow rate of 80 mL/min |
| | (1S)-1-(6-(2-methyl-2H- | | |
| | pyrazolo[3,4-b]pyridin-5- | | |
| | yl)thieno[2,3-b]pyridin-2-yl)-1- | | |
| | butanol | | |
| B27R | 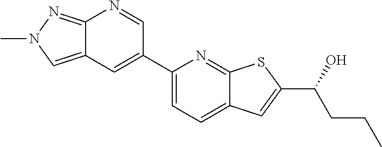 [SMILES: CCC[C@@H](O)c1cc2ccc(-c3cnc4nn(C)cc4c3)nc2s1] | 339.2; ¹H NMR (400 MHz, DMSO- d₆) δ 9.38 (d, J = 2.3 Hz, 1H) 8.91 (d, J = 2.3 Hz, 1H) 8.54 (s, 1H) 8.25 (d, J = 8.5 Hz, 1H) 8.08 (d, J = 8.3 Hz, 1H) 7.28 (s, 1H) 5.85 (d, J = 4.8 Hz, 1H) 4.94-4.86 (m, 1H) 4.24 (s, 3H) 1.82-1.69 (m, 2H) 1.49-1.31 (m, 2H) 0.93 (t, J = 7.4 Hz, 3H) | Peak 2/SFC: Chiralpak AS, 250 × 20 mm I.D., 5 μm, column Mobile phase: 70% CO₂: 30% MeOH(0.2% diethylamine, v/v) using a flow rate of 80 mL/min |
| | (1R)-1-(6-(2-methyl-2H- | | |
| | pyrazolo[3,4-b]pyridin-5- | | |
| | yl)thieno[2,3-b]pyridin-2-yl)-1- | | |
| | butanol | | |
| B29S | 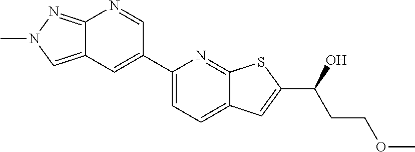 | 355.3; ¹H NMR (400 MHz, DMSO- d₆) δ 9.37 (d, J = 2.4 Hz, 1H), 8.90 (d, J = 2 Hz, 1H), 8.53 (s, 1H), 8.24 (d, J = 8.8 Hz, 1H), 8.07 (d, J = 7.6 Hz, 1H), 7.27 (s, 1H), 5.94 (br s, 1H), 4.99 (br s, 1H), 4.23 (s, 3H), 3.53-3.51 (m, 1H), 3.41-3.39 (m, 1H), 3.25 (s, 3H), 2.05-1.94 (m, 2H) | Peak 1/SFC: Chiralpak AD-3, 50 × 4.6 mm, 3 μm; Mobile phase: 50% CO₂: 50% MeOH; (0.1%IPAm, v/v) using a flow rate of 3 mL/min |
| | (1S)-3-methoxy-1-(6-(2-methyl- | | |
| | 2H-pyrazolo[3,4-b]pyridin-5- | | |
| | yl)thieno[2,3-b]pyridin-2-yl)-1- | | |
| | propanol | | |
| B29R | 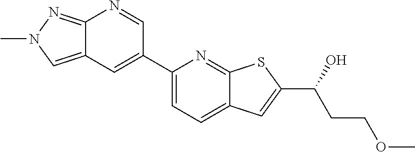 | 355.3; ¹H NMR (400 MHz, DMSO- d₆) δ 9.37 (d, J = 2.4 Hz, 1H), 8.90 (d, J = 2 Hz, 1H), 8.53 (s, 1H), 8.25 (d, J = 8.4 Hz, 1H), 8.08 (d, J = 8.8 Hz, 1H), 7.28 (s, 1H), 5.94 (br s, 1H), 4.99 (t, J = 6.4 Hz, 1H), 4.24 (s, 3H), 3.53-3.51 (m, 1H), 3.41- 3.39 (m, 1H), 3.25 (s, 3H), 2.05- 1.94 (m, 2H) | Peak 2/SFC: Chiralpak AD- 3(50 × 4.6 mm, 3 μm); Mobile phase: 50% CO₂: 50% MeOH; (0.1%IPAm, v/v) using a flow rate of 3 mL/min |
| | (1R)-3-methoxy-1-(6-(2-methyl- | | |
| | 2H-pyrazolo[3,4-b]pyridin-5- | | |
| | yl)thieno[2,3-b]pyridin-2-yl)-1- | | |
| | propanol | | |
| B72R | 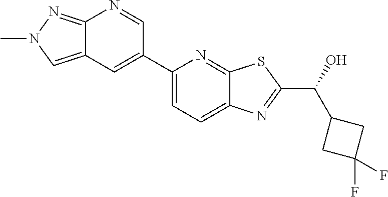 [SMILES: Cn1cc2cc(-c3ccc4nc([C@H](O)C5CC(F)(F)C5)sc4n3)cnc2n1] | 388.2; ¹H NMR (401 MHz, DMSO- d₆) δ 9.38 (d, J = 2.3 Hz, 1H), 8.95 (d, J = 2.3 Hz, 1H), 8.56 (s, 1H), 8.44 (d, J = 8.6 Hz, 1H), 8.24 (d, J = 8.7 Hz, 1H), 6.90 (d, J = 5.5 Hz, 1H), 5.01 (t, J = 5.0 Hz, 1H), 4.25 (s, 3H), 2.79-2.61 (m, 5H) | Peak 1/SFC: Chiralpak AS-H 250 × 30 mm, 5 μm; Mobile phase: 30% MeOH in CO₂ with a flow rate of 130 mL/min |
| | (R)-(3,3-difluorocyclobutyl)(5- | | |
| | (2-methyl-2H-pyrazolo[3,4- | | |
| | b]pyridin-5-yl)[1,3]thiazolo[5,4- | | |
| | b]pyridin-2-yl)methanol | | |
| B72S | 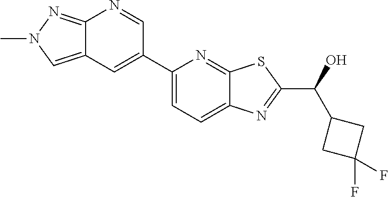 [SMILES: Cn1cc2cc(-c3ccc4nc([C@@H](O)C5CC(F)(F)C5)sc4n3)cnc2n1] | 388.2; ¹H NMR (401 MHz, DMSO- d₆) δ 9.39 (d, J = 2.4 Hz, 1H), 8.95 (d, J = 2.3 Hz, 1H), 8.56 (s, 1H), 8.44 (d, J = 8.6 Hz, 1H), 8.25 (d, J = 8.6 Hz, 1H), 6.90 (d, J = 5.5 Hz, 1H), 5.01 (t, J = 5.1 Hz, 1H), 4.25 (s, 3H), 2.69 (ddd, J = 15.5, 11.9, 6.9 Hz, 5H) | Peak 2/SFC: Chiralpak AS-H 250 × 30 mm, 5 μm; Mobile phase: 30% MeOH in CO₂ with a flow rate of 130 mL/min |
| | (S)-(3,3-difluorocyclobutyl)(5- | | |
| | (2-methyl-2H-pyrazolo[3,4- | | |
| | b]pyridin-5-yl)[1,3]thiazolo[5,4- | | |
| | b]pyridin-2-yl)methanol | | |
| B74S | 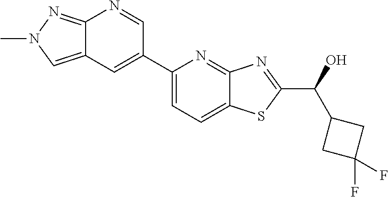 [SMILES: Cn1cc2cc(-c3ccc4sc([C@@H](O)C5CC(F)(F)C5)nc4n3)cnc2n1] | 388.2; ¹H NMR (400 MHz, DMSO- d₆) δ 9.42 (d, J = 2.3 Hz, 1H), 8.96 (d, J = 2.4 Hz, 1H), 8.70 (d, J = 8.4 Hz, 1H), 8.57 (s, 1H), 8.16 (d, J = 8.5 Hz, 1H), 6.92 (d, J = 5.5 Hz, 1H), 5.08 (t, J = 5.0 Hz, 1H), 4.25 (s, 3H), 2.84-2.59 (m, 5H) | Peak 1/SFC: Chiralcel OJ-H 250 × 4.6 mm, 5um; Mobile phase: 40% MeOH (0.2% NH₄OH) in CO₂ with a flow rate of 4.0 mL/min |
| | (S)-(3,3-difluorocyclobutyl)(5- | | |
| | (2-methyl-2H-pyrazolo[3,4- | | |
| | b]pyridin-5-yl)[1,3]thiazolo[4,5- | | |
| | b]pyridin-2-yl)methanol | | |
| B74R | 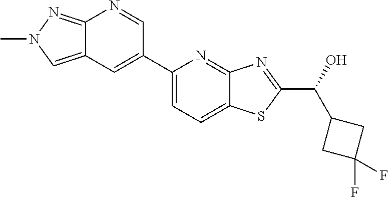 [SMILES: Cn1cc2cc(-c3ccc4sc([C@H](O)C5CC(F)(F)C5)nc4n3)cnc2n1] | 388.2; ¹H NMR (400 MHz, DMSO- d₆) δ 9.42 (d, J = 2.4 Hz, 1H), 8.96 (d, J = 2.4 Hz, 1H), 8.70 (d, J = 8.4 Hz, 1H), 8.57 (s, 1H), 8.16 (d, J =8.4 Hz, 1H), 6.92 (d, J = 5.6 Hz, 1H), 5.08 (t, J = 5.1 Hz, 1H), 4.25 (s, 3H), 2.89-2.60 (m, 5H) | Peak 2/SFC: Chiralcel OJ-H 250 × 4.6 mm, 5 μm; Mobile phase: 40% MeOH (0.2% NH₄OH) in CO₂ with a flow rate of 4.0 mL/min |
| | (R)-(3,3-difluorocyclobutyl)(5- | | |
| | (2-methyl-2H-pyrazolo[3,4- | | |
| | b]pyridin-5-yl)[1,3]thiazolo[4,5- | | |
| | b]pyridin-2-yl)methanol | | |
| D1R | 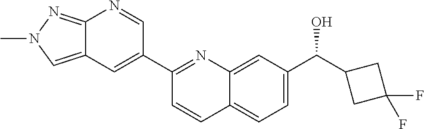 [SMILES: Cn1cc2cc(-c3ccc4ccc([C@H](O)C5CC(F)(F)C5)cc4n3)cnc2n1] | 381.1; ¹H NMR (401 MHz, DMSO- d₆) δ 9.55 (d, J = 2.3 Hz, 1H), 9.04 (d, J = 2.4 Hz, 1H), 8.59 (s, 1H), 8.46 (d, J = 8.7 Hz, 1H), 8.23 (d, J = 8.8 Hz, 1H), 8.05 (s, 1H), 7.97 (d, J = 8.4 Hz, 1H), 7.62 (dd, J = 8.3, 1.7 Hz, 1H), 5.80 (s, 1H), 4.81 (d, J = 5.0 Hz, 1H), 4.26 (s, 3H), 2.68- 2.43 (m, 5H) | Peak 1/SFC: Amylose-SA 250 × 30 mm, 5 μm; Mobile phase: 50% 1:1 (MeOH: 0.2% NH₄OH in ACN) in CO₂ with a flow rate of 160 mL/min |
| | (R)-(3,3-difluorocyclobutyl)(2- | | |
| | (2-methyl-2H-pyrazolo[3,4- | | |
| | b]pyridin-5-yl)-7- | | |
| | quinolinyl)methanol | | |
| D1S | 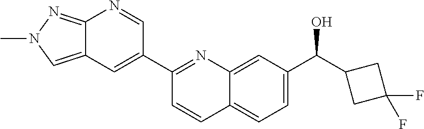 [SMILES: Cn1cc2cc(-c3ccc4ccc([C@@H](O)C5CC(F)(F)C5)cc4n3)cnc2n1] | 381.1; ¹H NMR (401 MHz, DMSO- d₆) δ 9.55 (d, J = 2.3 Hz, 1H), 9.03 (d, J = 2.5 Hz, 1H), 8.59 (s, 1H), 8.45 (d, J = 8.6 Hz, 1H), 8.23 (d, J = 8.7 Hz, 1H), 8.05 (s, 1H), 7.97 (d, J = 8.3 Hz, 1H), 7.62 (d, J = 8.4 Hz, 1H), 5.82 (s, 1H), 4.81 (d, J = 5.1 Hz, 1H), 4.26 (s, 3H), 2.61- 2.44 (m, 5H) | Peak 2/SFC: Amylose-SA 250 × 30 mm, 5 μm; Mobile phase: 50% 1:1 (MeOH: 0.2% NH₄OH in ACN) in CO₂ with a flow rate of 160 mL/min |
| | (S)-(3,3-difluorocyclobutyl)(2- | | |
| | (2-methyl-2H-pyrazolo[3,4- | | |
| | b]pyridin-5-yl)-7- | | |
| | quinolinyl)methanol | | |
| B84RC | 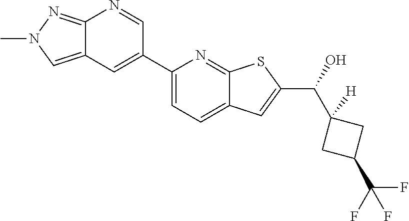 [SMILES: Cn1cc2cc(-c3ccc4cc([C@H](O)[C@H]5C[C@@H](C(F)(F)F)C5)sc4n3)cnc2n1] | 419.2; ¹H NMR (401 MHz, DMSO- d₆) δ 9.38 (d, J = 2.3 Hz, 1H), 8.91 (d, J = 2.3 Hz, 1H), 8.55 (s, 1H), 8.26 (d, J = 8.4 Hz, 1H), 8.09 (d, J = 8.4 Hz, 1H), 7.31 (s, 1H), 6.09 (d, J = 4.9 Hz, 1H), 4.81 (t, J = 5.8 Hz, 1H), 4.24 (s, 3H), 3.05 (h, J = 9.1 Hz, 1H), 2.68-2.59 (m, 1H), 2.17-2.03 (m, 4H) | Alternate condition (5) was used; After Step 1, SFC was performed; Peak 3/SFC: Chiralpak IG 250 × 50 mm, 5 μm; Mobile phase: 30% (1:1) MeOH:ACN in CO₂ with a flow rate of 150 mL/min |
| | (R)-(6-(2-methyl-2H- | | |
| | pyrazolo[3,4-b]pyridin-5- | | |
| | yl)thieno[2,3-b]pyridin-2- | | |
| | yl)((cis)-3- | | |
| | (trifluoromethyl)cyclobutyl) | | |
| | methanol | | |
| B84SC | 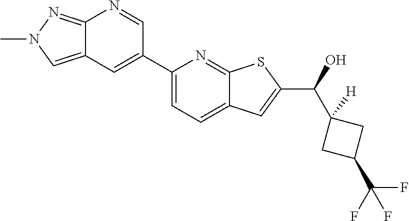 [SMILES: Cn1cc2cc(-c3ccc4cc([C@@H](O)[C@H]5C[C@@H](C(F)(F)F)C5)sc4n3)cnc2n1] | 419.2; ¹H NMR (401 MHz, DMSO- d₆) δ 9.38 (d, J = 2.3 Hz, 1H), 8.91 (d, J = 2.3 Hz, 1H), 8.55 (s, 1H), 8.26 (d, J = 8.4 Hz, 1H), 8.09 (d, J = 8.4 Hz, 1H), 7.31 (s, 1H), 6.16 (d, J = 5.0 Hz, 1H), 4.98-4.90 (m, 1H), 4.24 (s, 3H), 3.18-3.05 (m, 1H), 2.71 (q, J = 8.2 Hz, 1H), 2.39- 2.15 (m, 1H), 2.15 (d, J = 3.1 Hz, 1H), 2.13-2.06 (m, 2H). | Alternate condition (5) was used; After Step 1, SFC was performed; Peak 2/SFC: Chiralpak IG 250 × 50 mm, 5 μm; Mobile phase: 30% (1:1) MeOH:ACN in CO₂ with a flow rate of 150 mL/min |
| | (S)-(6-(2-methyl-2H- | | |
| | pyrazolo[3,4-b]pyridin-5- | | |
| | yl)thieno[2,3-b]pyridin-2- | | |
| | yl)((cis)-3- | | |
| | (trifluoromethyl)cyclobutyl) | | |
| | methanol | | |
| B84RT | 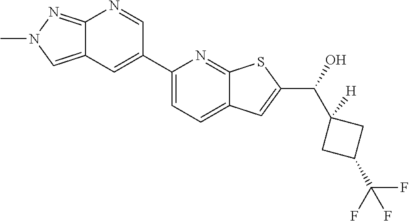 [SMILES: Cn1cc2cc(-c3ccc4cc([C@H](O)[C@H]5C[C@H](C(F)(F)F)C5)sc4n3)cnc2n1] | 419.2; ¹H NMR (401 MHz, DMSO- d₆) δ 9.38 (d, J = 2.3 Hz, 1H), 8.91 (d, J = 2.3 Hz, 1H), 8.55 (s, 1H), 8.26 (d, J = 8.5 Hz, 1H), 8.09 (d, J = 8.4 Hz, 1H), 7.33-7.28 (m, 1H), 6.16 (d, J = 5.0 Hz, 1H), 4.98-4.90 (m, 1H), 4.24 (s, 3H), 3.11 (dq, J = 10.2, 5.0 Hz, 1H), 2.72 (q, J = 8.1 Hz, 1H), 2.39-2.24 (m, 1H), 2.27-2.18 (m, 1H), 2.13 (ddd, J = 13.2, 8.7, 5.8 Hz, 2H) | Alternate condition (5) was used; After Step 1, SFC was performed; Peak 4/SFC: Chiralpak IG 250 × 50 mm, 5 μm; Mobile phase: 30% (1:1) MeOH:ACN in CO₂ with a flow rate of 150 mL/min |
| | (R)-(6-(2-methyl-2H- | | |
| | pyrazolo[3,4-b]pyridin-5- | | |
| | yl)thieno[2,3-b]pyridin-2- | | |
| | yl)((trans)-3- | | |
| | (trifluoromethyl)cyclobutyl) | | |
| | methanol | | |
| B84ST | 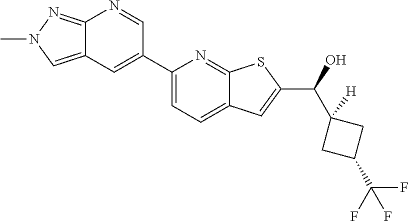 [SMILES: Cn1cc2cc(-c3ccc4cc([C@@H](O)[C@H]5C[C@H](C(F)(F)F)C5)sc4n3)cnc2n1] | 419.2; ¹H NMR (401 MHz, DMSO- d₆) δ 9.38 (d, J = 2.3 Hz, 1H), 8.91 (d, J = 2.3 Hz, 1H), 8.55 (s, 1H), 8.26 (d, J = 8.4 Hz, 1H), 8.09 (d, J = 8.4 Hz, 1H), 7.31 (s, 1H), 6.09 (d, J = 4.9 Hz, 1H), 4.80 (dd, J = 6.6, 4.9 Hz, 1H), 4.24 (s, 3H), 3.05 (h, J = 9.2 Hz, 1H), 2.62 (dd, J = 16.4, 8.2 Hz, 1H), 2.17-2.03 (m, 4H) | Alternate condition (5) was used; After Step 1, SFC was performed; Peak 1/SFC: Chiralpak IG 250 × 50 mm, 5 μm; Mobile phase: 30% (1:1) MeOH:ACN in CO₂ with a flow rate of 150 mL/min |
| | (S)-(6-(2-methyl-2H- | | |
| | pyrazolo[3,4-b]pyridin-5- | | |
| | yl)thieno[2,3-b]pyridin-2- | | |
| | yl)((trans)-3- | | |
| | (trifluoromethyl)cyclobutyl) | | |
| | methanol | | |
| B85SS | 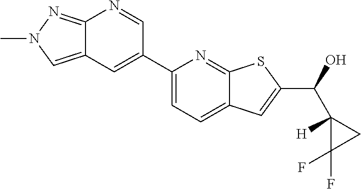 [SMILES: Cn1cc2cc(-c3ccc4cc([C@@H](O)[C@@H]5CC5(F)F)sc4n3)cnc2n1] | 373.2; ¹H NMR (400 MHz, DMSO- d₆) δ 9.40 (d, J = 2.3 Hz, 1H), 8.93 (d, J = 2.3 Hz, 1H), 8.55 (s, 1H), 8.33 (d, J = 8.4 Hz, 1H), 8.12 (d, J = 8.4 Hz, 1H), 7.31 (d, J = 1.1 Hz, 1H), 6.37 (d, J = 5.5 Hz, 1H), 4.68 (dd, J = 8.8, 5.8 Hz, 1H), 4.25 (s, 3H), 2.26-2.11 (m, 1H), 1.81 (dtd, J = 11.6, 7.7, 3.6 Hz, 1H), 1.65 (dtd, J = 11.9, 7.6, 3.7 Hz, 1H) | Alternate condition (5) was used; After Step 1, the sample was purified by chromatography to provide a 1st eluting isomer and a 2nd eluting isomer; The 2nd eluting isomer was purified via SFC; Peak 1/SFC: Chiralpak IG 250 × 50 mm, 5 μm; Mobile phase: 40% (1:1) MeOH:ACN in CO₂ with a flow rate of 150 mL/min |
| | (S)-((1S)-2,2- | | |
| | difluorocyclopropyl)(6-(2- | | |
| | methyl-2H-pyrazolo[3,4- | | |
| | b]pyridin-5-yl)thieno[2,3- | | |
| | b]pyridin-2-yl)methanol | | |
| B85RR | 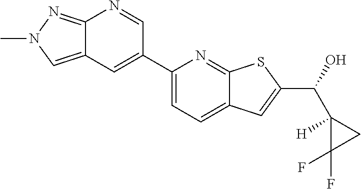 [SMILES: Cn1cc2cc(-c3ccc4cc([C@H](O)[C@H]5CC5(F)F)sc4n3)cnc2n1] | 373.2; ¹H NMR (401 MHz, DMSO- d₆) δ 9.40 (d, J = 2.3 Hz, 1H), 8.93 (d, J = 2.4 Hz, 1H), 8.55 (s, 1H), 8.34 (d, J = 8.4 Hz, 1H), 8.12 (d, J = 8.4 Hz, 1H), 7.31 (d, J = 1.2 Hz, 1H), 6.36 (d, J = 5.5 Hz, 1H), 4.68 (dd, J = 8.9, 5.9 Hz, 1H), 4.25 (s, 3H), 2.19 (dddd, J = 14.2, 11.4, 9.1, 7.6 Hz, 1H), 1.81 (dtd, J = 11.5, 7.6, 3.7 Hz, 1H), 1.76-1.60 (m, 1H) | Alternate condition (5) was used; After Step 1, the sample was purified by chromatography to provide a 1st eluting isomer and a 2nd eluting isomer; The 2nd eluting isomer was purified via SFC; Peak 2/SFC: Chiralpak IG 250 × 50 mm, 5 μm; Mobile phase: 40% (1:1) MeOH:ACN in CO₂ with a flow rate of 150 mL/min |
| | (R)-((1R)-2,2- | | |
| | difluorocyclopropyl)(6-(2- | | |
| | methyl-2H-pyrazolo[3,4- | | |
| | b]pyridin-5-yl)thieno[2,3- | | |
| | b]pyridin-2-yl)methanol | | |
| B85RS | 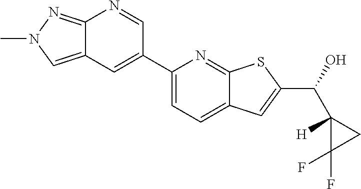 [SMILES: Cn1cc2cc(-c3ccc4cc([C@H](O)[C@@H]5CC5(F)F)sc4n3)cnc2n1] | 373.2; ¹H NMR (400 MHz, DMSO- d₆) δ 9.39 (d, J = 2.3 Hz, 1H), 8.92 (d, J = 2.4 Hz, 1H), 8.55 (s, 1H), 8.28 (d, J = 8.4 Hz, 1H), 8.10 (d, J = 8.4 Hz, 1H), 7.40 (s, 1H), 6.42 (d, J = 5.4 Hz, 1H), 4.68 (dd, J = 9.6, 5.5 Hz, 1H), 4.25 (s, 3H), 2.23- 2.05 (m, 1H), 1.72 (ddt, J = 11.9, 8.7, 4.5 Hz, 1H), 1.68-1.51 (m, 1H) | Alternate condition (5) was used; After Step 1, the sample was purified by chromatography to provide a 1st eluting isomer and a 2nd eluting isomer; The 1st eluting isomer was purified via SFC; Peak 1/SFC: Chiralpak IG 250 × 50 mm, 5 μm; Mobile phase: 30% (1:1) MeOH:ACN in CO₂ with a flow rate of 150 mL/min |
| | (R)-((1S)-2,2- | | |
| | difluorocyclopropyl)(6-(2- | | |
| | methyl-2H-pyrazolo[3,4- | | |
| | b]pyridin-5-yl)thieno[2,3- | | |
| | b]pyridin-2-yl)methanol | | |
| B85SR | 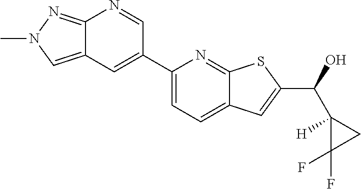 [SMILES: Cn1cc2cc(-c3ccc4cc([C@@H](O)[C@H]5CC5(F)F)sc4n3)cnc2n1] | 373.2; ¹H NMR (400 MHz, DMSO- d₆) δ 9.39 (d, J = 2.3 Hz, 1H), 8.92 (d, J = 2.4 Hz, 1H), 8.55 (s, 1H), 8.28 (d, J = 8.4 Hz, 1H), 8.10 (d, J = 8.4 Hz, 1H), 7.40 (s, 1H), 6.42 (d, J = 5.4 Hz, 1H), 4.68 (dd, J = 9.5, 5.5 Hz, 1H), 4.25 (s, 3H), 2.18 (qd, J = 12.2, 8.3 Hz, 1H), 1.71 (tdd, J = 12.1, 7.7, 4.4 Hz, 1H), 1.60 (ddd, J = 13.0, 7.7, 4.0 Hz, 1H) | Alternate condition (5) was used; After Step 1, the sample was purified by chromatography to provide a 1st eluting isomer and a 2nd eluting isomer; The 1st eluting isomer was purified via SFC; Peak 2/SFC: Chiralpak IG 250 × 50 mm, 5 μm; Mobile phase: 30% (1:1) MeOH:ACN in CO₂ with a flow rate of 150 mL/min |
| | (S)-((1R)-2,2- | | |
| | difluorocyclopropyl)(6-(2- | | |
| | methyl-2H-pyrazolo[3,4- | | |
| | b]pyridin-5-yl)thieno[2,3- | | |
| | b]pyridin-2-yl)methanol | | |
| B88RC | 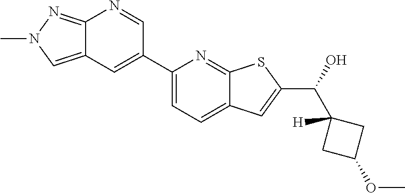 | 381.2; ¹H NMR (401 MHz, DMSO- d₆) δ 9.38 (d, J = 2.3 Hz, 1H), 8.91 (d, J = 2.3 Hz, 1H), 8.55 (s, 1H), 8.25 (d, J = 8.5 Hz, 1H), 8.08 (d, J = 8.4 Hz, 1H), 7.27 (s, 1H), 5.97 (d, J = 4.9 Hz, 1H), 4.79 (t, J = 5.4 Hz, 1H), 4.24 (s, 3H), 3.70 (p, J = 7.2 Hz, 1H), 3.12 (s, 3H), 2.30- 2.10 (m, 3H), 1.87-1.79 (m, 2H) | Alternate condition (5) was used; After Step 1, the sample was purified via SFC Peak 2/SFC: Chiralpak IG 250 × 50 mm, 5 μm; Mobile phase: 35% (1:1) MeOH:ACN in CO₂ with a flow rate of 150 mL/min The sample was purified by SFC Peak 2/SFC: Chiralpak IG 250 × 50 mm, 5 μm; Mobile phase: 35% (1:1) MeOH:ACN in CO2 with a flow rate of 5 mL/min |
| | (R)-((cis)-3- | | |
| | methoxycyclobutyl)(6-(2- | | |
| | methyl-2H-pyrazolo[3,4- | | |
| | b]pyridin-5-yl)thieno[2,3- | | |
| | b]pyridin-2-yl)methanol | | |
| B88SC | 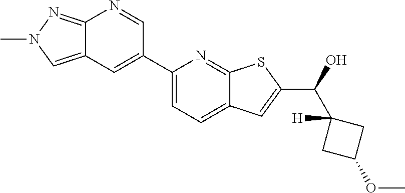 | 381.2; ¹H NMR (401 MHz, DMSO- d₆) δ 9.38 (d, J = 2.3 Hz, 1H), 8.91 (d, J = 2.3 Hz, 1H), 8.55 (s, 1H), 8.25 (d, J = 8.4 Hz, 1H), 8.08 (d, J = 8.4 Hz, 1H), 7.27 (s, 1H), 5.97 (d, J = 4.9 Hz, 1H), 4.79 (t, J = 5.4 Hz, 1H), 4.24 (s, 3H), 3.70 (p, J = 7.2 Hz, 1H), 3.12 (s, 3H), 2.30- 2.10 (m, 3H), 1.87-1.79 (m, 2H) | Alternate condition (5) was used; After Step 1, the sample was purified via SFC Peak 2/SFC: Chiralpak IG 250 × 50 mm, 5 μm; Mobile phase: 35% (1:1) MeOH:ACN in CO₂ with a flow rate of 150 mL/min The sample was purified by SFC Peak 1/SFC: Chiralpak IG 250 × 50 mm, 5 μm; Mobile phase: 35% (1:1) MeOH:ACN in CO₂ with a flow rate of 5 mL/min |
| | (S)-((cis)-3- | | |
| | methoxycyclobutyl)(6-(2- | | |
| | methyl-2H-pyrazolo[3,4- | | |
| | b]pyridin-5-yl)thieno[2,3- | | |
| | b]pyridin-2-yl)methanol | | |
| B88RT | 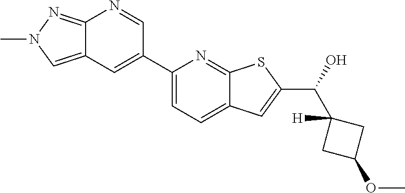 | 381.2; ¹H NMR (401 MHz, DMSO- d₆) δ 9.38 (d, J = 2.3 Hz, 1H), 8.91 (d, J = 2.3 Hz, 1H), 8.55 (s, 1H), 8.26 (d, J = 8.4 Hz, 1H), 8.09 (d, J = 8.4 Hz, 1H), 7.29 (s, 1H), 6.03 (d, J = 4.9 Hz, 1H), 4.86 (dd, J = 7.1, 4.9 Hz, 1H), 4.24 (s, 3H), 3.95 (p, J = 6.1 Hz, 1H), 3.12 (s, 3H), 2.59- 2.52 (m, 1H), 2.30-2.19 (m, 1H), 2.17-2.08 (m, 1H), 2.03-1.96 (m, 2H) | Alternate condition (5) was used; After Step 1, the sample was purified via SFC Peak 1/SFC: Chiralpak IG 250 × 50 mm, 5 μm; Mobile phase: 35% (1:1) MeOH:ACN in CO₂ with a flow rate of 150 mL/min The sample was purified by SFC Peak 2/SFC: Chiralpak IG 250 × 50 mm, 5 μm; Mobile phase: 35% (1:1) MeOH:ACN in CO₂ with a flow rate of 5 mL/min |
| | (R)-((trans)-3- | | |
| | methoxycyclobutyl)(6-(2- | | |
| | methyl-2H-pyrazolo[3,4- | | |
| | b]pyridin-5-yl)thieno[2,3- | | |
| | b]pyridin-2-yl)methanol | | |
| B88ST | 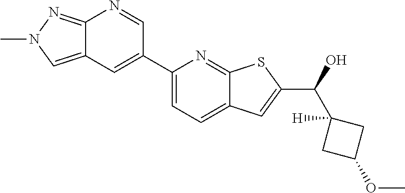 | 381.2; ¹H NMR (401 MHz, DMSO- d₆) δ 9.38 (d, J = 2.3 Hz, 1H), 8.91 (d, J = 2.3 Hz, 1H), 8.55 (s, 1H), 8.26 (d, J = 8.4 Hz, 1H), 8.09 (d, J = 8.5 Hz, 1H), 7.29 (s, 1H), 6.03 (d, J = 4.9 Hz, 1H), 4.86 (dd, J = 7.1, 4.9 Hz, 1H), 4.24 (s, 3H), 3.95 (p, J = 6.2 Hz, 1H), 3.12 (s, 3H), 2.59- 2.52 (m, 1H), 2.30-2.19 (m, 1H), 2.17-2.08 (m, 1H), 2.03-1.96 (m, 2H) | Alternate condition (5) was used; After Step 1, the sample was purified via SFC Peak 1/SFC: Chiralpak IG 250 × 50 mm, 5 μm; Mobile phase: 35% (1:1) MeOH:ACN in CO₂ with a flow rate of 150 mL/min The sample was purified by SFC Peak 1/SFC: Chiralpak IG 250 × 50 mm, 5 μm; Mobile phase: 35% (1:1) MeOH:ACN in CO₂ with a flow rate of 5 mL/min |
| | (S)-((trans)-3- | | |
| | methoxycyclobutyl)(6-(2- | | |
| | methyl-2H-pyrazolo[3,4- | | |
| | b]pyridin-5-yl)thieno[2,3- | | |
| | b]pyridin-2-yl)methanol | | |
| C1 | 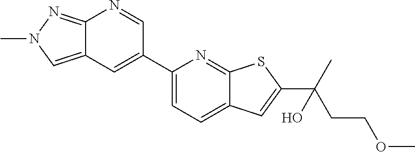 | 369.1; ¹H NMR (400 MHz, DMSO- d₆) δ 9.37 (d, J = 2.0 Hz, 1H), 8.90 (d, J = 2.0 Hz, 1H), 8.53 (s, 1H), 8.23 (d, J = 8.0 Hz, 1H), 8.07 (d, J = 8.4 Hz, 1H), 7.24 (s, 1H), 5.76 (s, 1H), 4.23 (s, 3H), 3.50-3.33 (m, 2H), 3.17 (s, 3H), 2.09 (t, J = 6.8 Hz, 2H), 1.60 (s, 3H) | |
| | 4-methoxy-2-(6-(2-methyl-2H- | | |
| | pyrazolo[3,4-b]pyridin-5- | | |
| | yl)thieno[2,3-b]pyridin-2-yl)-2- | | |
| | butanol | | |
| C2 | 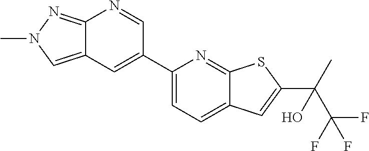 [SMILES: Cn1cc2cc(-c3ccc4cc(C(C)(O)C(F)(F)F)sc4n3)cnc2n1] | 379.0; ¹H NMR (400 MHz, DMSO- d₆) δ 9.39 (d, J = 2.4 Hz, 1H), 8.92 (d, J = 2.0 Hz, 1H), 8.55 (s, 1H), 8.35 (d, J = 8.4 Hz, 1H), 8.14 (d, J = 8.8 Hz, 1H), 7.55 (s, 1H), 7.41 (s, 1H), 4.24 (s, 3H), 1.83(s, 3H) | |
| | 1,1,1-trifluoro-2-(6-(2-methyl- | | |
| | 2H-pyrazolo[3,4-b]pyridin-5- | | |
| | yl)thieno[2,3-b]pyridin-2-yl)-2- | | |
| | propanol | | |
| C3 | 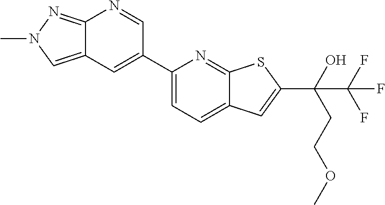 [SMILES: COCCC(O)(c1cc2ccc(-c3cnc4nn(C)cc4c3)nc2s1)C(F)(F)F] | 423.1; ¹H NMR (400 MHz, DMSO- d₆) δ 9.39 (d, J = 2.0 Hz, 1H), 8.94 (d, J = 2.0 Hz, 1H), 8.56 (s, 1H), 8.35 (d, J = 8.4 Hz, 1H), 8.14 (d, J = 8.4 Hz, 1H), 7.56 (s, 1H), 7.35 (s, 1H), 4.24 (s, 3H), 3.55-3.49 (m, 1H), 3.31-3.25 (m, 1H), 3.16 (s, 3H), 2.48-2.32 (m, 2H) | |
| | 1,1,1-trifluoro-4-methoxy-2-(6- | | |
| | (2-methyl-2H-pyrazolo[3,4- | | |
| | b]pyridin-5-yl)thieno[2,3- | | |
| | b]pyridin-2-yl)-2-butanol | | |
| C4 | 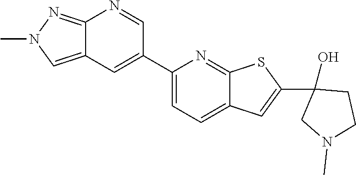 [SMILES: CN1CCC(O)(c2cc3ccc(-c4cnc5nn(C)cc5c4)nc3s2)C1] | 366.1; ¹H NMR (400 MHz, DMSO- d₆) δ 9.37 (d, J = 2.0 Hz, 1H), 8.89 (d, J = 2.4 Hz, 1H), 8.53 (s, 1H), 8.23 (d, J = 8.4 Hz, 1H), 8.06 (d, J = 8.4 Hz, 1H), 7.31 (s, 1H), 6.06 (s, 1H), 4.23 (s, 3H), 2.96 (d, J = 9.2 Hz, 1H), 2.80-2.65 (m, 3H), 2.32 (s, 3H), 2.32-2.25 (m, 1H), 2.10-2.05 (m, 1H) | |
| | 1-methyl-3-(6-(2-methyl-2H- | | |
| | pyrazolo[3,4-b]pyridin-5- | | |
| | yl)thieno[2,3-b]pyridin-2-yl)-3- | | |
| | pyrrolidinol | | |
| C6 | 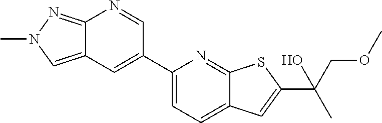 | 355.1; ¹H NMR (400 MHz, DMSO- d₆) δ 9.37 (d, J = 2.0 Hz, 1H), 8.90 (d, J = 2.4 Hz, 1H), 8.54 (s, 1H), 8.24 (d, J = 8.8 Hz, 1H), 8.07 (d, J = 8.4 Hz, 1H), 7.27 (s, 1H), 5.88 (s, 1H), 4.24 (s, 3H), 3.50 (s, 2H), 3.31 (s, 3H), 1.57 (s, 3H) | |
| | 1-methoxy-2-(6-(2-methyl-2H- | | |
| | pyrazolo[3,4-b]pyridin-5- | | |
| | yl)thieno[2,3-b]pyridin-2-yl)-2- | | |
| | propanol | | |
| C7 | 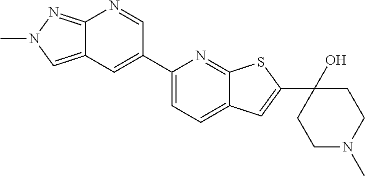 | 380.1; ¹H NMR (400 MHz, DMSO- d₆) δ 9.37 (s, 1H), 8.91 (s, 1H), 8.54 (s, 1H), 8.25 (d, J = 7.6 Hz, 1H), 8.09 (d, J = 8.4 Hz, 1H), 7.31 (s, 1H), 5.91-5.71 (m, 1H), 4.23 (s, 3H), 2.82-2.65 (m, 2H), 2.41- 2.25 (m, 2H), 2.15-2.01 (m, 2H), 2.01-1.85 (m, 2H) | |
| | 1-methyl-4-(6-(2-methyl-2H- | | |
| | pyrazolo[3,4-b]pyridin-5- | | |
| | yl)thieno[2,3-b]pyridin-2-yl)-4- | | |
| | piperidinol | | |
| C8 | 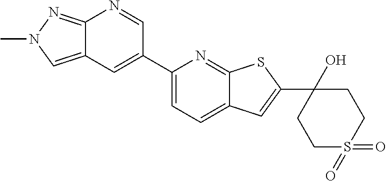 [SMILES: Cn1cc2cc(-c3ccc4cc(C5(O)CCS(=O)(=O)CC5)sc4n3)cnc2n1] | 415.0; ¹H NMR (400 MHz, DMSO- d₆) δ 9.38 (d, J = 2.4 Hz, 1H), 8.92 (d, J = 2.4 Hz, 1H), 8.54 (s, 1H), 8.27 (d, J = 8.0 Hz, 1H), 8.11 (d, J = 8.8 Hz, 1H), 7.41 (s, 1H), 6.46 (s, 1H), 4.23 (s, 3H), 3.45-3.38 (m, 2H), 3.17-3.15 (m, 2H), 2.5-2.45 (m, 2H), 2.35-2.33 (m, 2H) | |
| | 4-(6-(2-methyl-2H-pyrazolo[3,4- | | |
| | b]pyridin-5-yl)thieno[2,3- | | |
| | b]pyridin-2-yl)tetrahydro-2H- | | |
| | thiopyran-4-ol 1,1-dioxide | | |
| C9 | 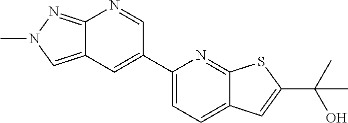 [SMILES: Cn1cc2cc(-c3ccc4cc(C(C)(C)O)sc4n3)cnc2n1] | 325.0; ¹H NMR (400 MHz, DMSO- d₆) δ 9.37 (d, J = 2.4 Hz, 1H), 8.90 (d, J = 2.0 Hz, 1H), 8.53 (s, 1H), 8.22 (d, J = 8.4 Hz, 1H), 8.06 (d, J = 8.4 Hz, 1H), 7.23 (s, 1H), 5.76 (s, 1H), 4.23 (s, 3H), 1.59 (s, 6H) | |
| | 2-(6-(2-methyl-2H-pyrazolo[3,4- | | |
| | b]pyridin-5-yl)thieno[2,3- | | |
| | b]pyridin-2-yl)-2-propanol | | |
| C10 | 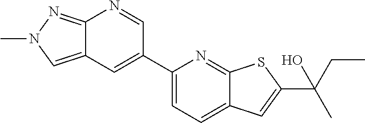 [SMILES: CCC(C)(O)c1cc2ccc(-c3cnc4nn(C)cc4c3)nc2s1] | 339.0; ¹H NMR (400 MHz, DMSO- d₆) δ 9.37 (d, J = 2.4 Hz, 1H), 8.89 (d, J = 2.0 Hz, 1H), 8.53 (s, 1H), 8.22 (d, J = 8.4 Hz, 1H), 8.06 (d, J = 9.2 Hz, 1H), 7.19 (s, 1H), 5.61 (s, 1H), 4.23 (s, 3H), 1.84-1.82 (m, 2H), 1.55 (s, 3H), 0.84 (t, J = 7.2 Hz, 3H) | |
| | 2-(6-(2-methyl-2H-pyrazolo[3,4- | | |
| | b]pyridin-5-yl)thieno[2,3- | | |
| | b]pyridin-2-yl)-2-butanol | | |
| C11 | 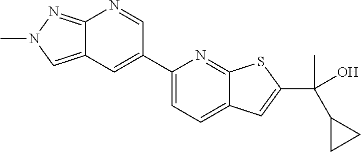 [SMILES: Cn1cc2cc(-c3ccc4cc(C(C)(O)C5CC5)sc4n3)cnc2n1] | 351.1; ¹H NMR (400 MHz, DMSO- d₆) δ 9.37 (d, J = 2.4 Hz, 1H), 8.89 (d, J = 2.3 Hz, 1H), 8.53 (s, 1H), 8.23 (d, J = 8.4 Hz, 1H), 8.06 (d, J = 8.4 Hz, 1H), 7.28 (s, 1H), 5.46 (s, 1H), 4.23 (s, 3H), 1.58 (s, 3H), 1.35- 1.25 (m, 1H), 0.62-0.32 (m, 4H) | |
| | 1-cyclopropyl-1-(6-(2-methyl- | | |
| | 2H-pyrazolo[3,4-b]pyridin-5- | | |
| | yl)thieno[2,3-b]pyridin-2- | | |
| | yl)ethanol | | |
| C12 | 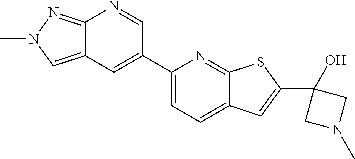 | 352.0; ¹H NMR (400 MHz, DMSO- d₆) δ 9.38 (d, J = 2.4 Hz, 1H), 8.91 (d, J = 2.4 Hz, 1H), 8.54 (s, 1H), 8.27 (d, J = 8.4 Hz, 1H), 8.09 (d, J = 8.4 Hz, 1H), 7.49 (s, 1H), 6.57 (s, 1H), 4.24 (s, 3H), 3.66 (d, J = 7.2 Hz, 2H), 3.33-3.26 (m, 2H), 2.38 (s, 3H) | |
| | 1-methyl-3-(6-(2-methyl-2H- | | |
| | pyrazolo[3,4-b]pyridin-5- | | |
| | yl)thieno[2,3-b]pyridin-2-yl)-3- | | |
| | azetidinol | | |
| C13 | 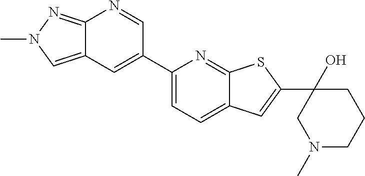 [SMILES: CN1CCCC(O)(c2cc3ccc(-c4cnc5nn(C)cc5c4)nc3s2)C1] | 380.1; ¹H NMR (400 MHz, DMSO- d₆) δ 9.38 (d, J = 2.0 Hz, 1H), 8.90 (d, J = 2.4 Hz, 1H), 8.54 (s, 1H), 8.25 (d, J = 8.4 Hz, 1H), 8.07 (d, J = 8.4 Hz, 1H), 7.43 (s, 1H), 5.66 (br s, 1H), 4.24 (s, 3H), 2.74-2.62 (m, 1H), 2.37-2.33 (m, 3H), 2.26 (s, 3H), 1.88-1.75 (m, 3H), 1.72- 1.51 (m, 1H) | |
| | 1-methyl-3-(6-(2-methyl-2H- | | |
| | pyrazolo[3,4-b]pyridin-5- | | |
| | yl)thieno[2,3-b]pyridin-2-yl)-3- | | |
| | piperidinol | | |
| C14 | 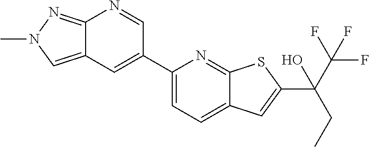 [SMILES: CCC(O)(c1cc2ccc(-c3cnc4nn(C)cc4c3)nc2s1)C(F)(F)F] | 393.0; ¹H NMR (400 MHz, DMSO- d₆) δ 9.38 (br d, J = 0.9 Hz, 1H), 8.96-8.88 (m, 1H), 8.56 (s, 1H), 8.35-8.14 (m, 2H), 7.50 (s, 1H), 7.14 (d, J = 4.4 Hz, 1H), 4.25 (s, 3H), 2.22-2.11 (m, 2H), 0.88- 0.83 (m, 3H) | |
| | 1,1,1-trifluoro-2-(6-(2-methyl- | | |
| | 2H-pyrazolo[3,4-b]pyridin-5- | | |
| | yl)thieno[2,3-b]pyridin-2-yl)-2- | | |
| | butanol | | |
| C15 | 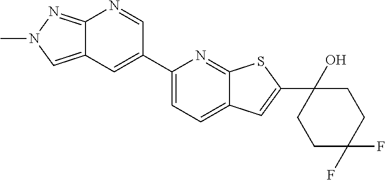 [SMILES: Cn1cc2cc(-c3ccc4cc(C5(O)CCC(F)(F)CC5)sc4n3)cnc2n1] | 401.1; ¹H NMR (400 MHz, DMSO- d₆) δ 9.38 (s, 1H), 8.91 (s, 1H), 8.54 (s, 1H), 8.25 (d, J = 8.8 Hz, 1H), 8.10 (d, J = 8.0 Hz, 1H), 7.35 (s, 1H), 6.06 (s, 1H), 4.24 (s, 3H), 2.26- 2.18 (m, 2H), 2.08-2.01 (m, 6H) | |
| | 4,4-difluoro-1-(6-(2-methyl-2H- | | |
| | pyrazolo[3,4-b]pyridin-5- | | |
| | yl)thieno[2,3-b]pyridin-2- | | |
| | yl)cyclohexanol | | |
| C16 | 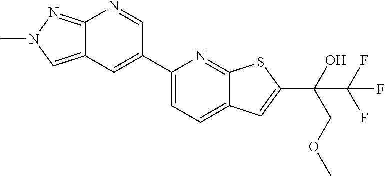 [SMILES: COCC(O)(c1cc2ccc(-c3cnc4nn(C)cc4c3)nc2s1)C(F)(F)F] | 409.1; ¹H NMR (400 MHz, DMSO- d₆) δ 9.39 (d, J = 2.0 Hz, 1H), 8.94 (d, J = 2.4 Hz, 1H), 8.56 (s, 1H), 8.36 (d, J = 8.4 Hz, 1H), 8.14 (d, J = 8.4 Hz, 1H), 7.57 (s,1H), 7.55 (s, 1H), 4.24 (s, 3H), 3.95-3.86 (m, 2H), 3.36 (s, 3H) | |
| | 1, 1,1-trifluoro-3-methoxy-2-(6- | | |
| | (2-methyl-2H-pyrazolo[3,4- | | |
| | b]pyridin-5-yl)thieno[2,3- | | |
| | b]pyridin-2-yl)-2-propanol | | |
| C17 | 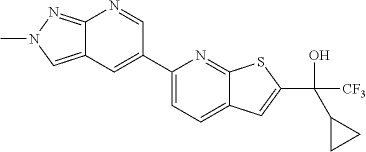 [SMILES: Cn1cc2cc(-c3ccc4cc(C(O)(C5CC5)C(F)(F)F)sc4n3)cnc2n1] | 405.0; ¹H NMR (400 MHz, DMSO- d₆) δ 9.39 (d, J = 2.4 Hz, 1H), 8.94 (d, J = 2.4 Hz, 1H), 8.56 (s, 1H), 8.37 (d, J = 8.8 Hz, 1H), 8.15 (d, J = 8.4 Hz, 1H), 7.61 (s, 1H), 6.92 (s, 1H), 4.24 (s, 3H), 1.66-1.63 (m, 1H), 0.74-0.61 (m, 1H), 0.57- 0.54 (m, 3H) | |
| | 1-cyclopropyl-2,2,2-trifluoro-1- | | |
| | (6-(2-methyl-2H-pyrazolo[3,4- | | |
| | b]pyridin-5-yl)thieno[2,3- | | |
| | b]pyridin-2-yl)ethanol | | |
| C18 | 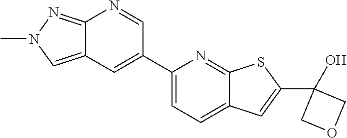 [SMILES: Cn1cc2cc(-c3ccc4cc(C5(O)COC5)sc4n3)cnc2n1] | 339.0; ¹H NMR (400 MHz, DMSO- d₆) δ 9.39 (d, J = 2.4 Hz, 1H), 8.93 (d, J = 2.4 Hz, 1H), 8.55 (s, 1H), 8.32 (d, J = 8.4 Hz, 1H), 8.13 (d, J = 8.4 Hz, 1H), 7.6 (s, 1H), 7.05 (s, 1H), 4.85-4.8 (m, 4H), 4.24 (s, 3H) | |
| | 3-(6-(2-methyl-2H-pyrazolo[3,4- | | |
| | b]pyridin-5-yl)thieno[2,3- | | |
| | b]pyridin-2-yl)-3-oxetanol | | |
| C19 | 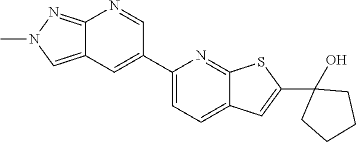 [SMILES: Cn1cc2cc(-c3ccc4cc(C5(O)CCCC5)sc4n3)cnc2n1] | 351.0; ¹H NMR (400 MHz, DMSO- d₆) δ 9.37 (d, J = 2.1 Hz, 1H), 8.90 (d, J = 2.1 Hz, 1H), 8.53 (s, 1H), 8.22 (d, J = 8.3 Hz, 1H), 8.07 (d, J = 8.5 Hz, 1H), 7.28 (s, 1H), 5.62 (s, 1H), 4.24 (s, 3H), 2.08-1.95 (m, 4H), 1.95-1.72 (m, 4H) | |
| | 1-(6-(2-methyl-2H-pyrazolo[3,4- | | |
| | b]pyridin-5-yl)thieno[2,3- | | |
| | b]pyridin-2-yl)cyclopentanol | | |
| C20 | 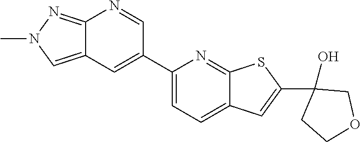 [SMILES: Cn1cc2cc(-c3ccc4cc(C5(O)CCOC5)sc4n3)cnc2n1] | 353.0; ¹H NMR (400 MHz, DMSO- d₆) δ 9.38 (d, J = 2.0 Hz, 1H), 8.91 (d, J = 2.0 Hz, 1H), 8.54 (s, 1H), 8.26 (d, J = 8.4 Hz, 1H), 8.09 (d, J = 8.4 Hz, 1H), 7.38 (s, 1H), 6.22 (s, 1H), 4.24 (s, 3H), 4.05-4.02 (m, 2H), 3.90 (d, J = 2.0 Hz, 2H), 2.42- 2.30 (m, 2H) | |
| | 3-(6-(2-methyl-2H-pyrazolo[3,4- | | |
| | b]pyridin-5-yl)thieno[2,3- | | |
| | b]pyridin-2-yl)tetrahydro-3- | | |
| | furanol | | |
| C21 | 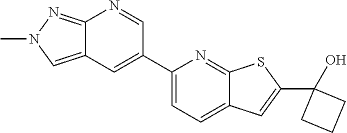 [SMILES: Cn1cc2cc(-c3ccc4cc(C5(O)CCC5)sc4n3)cnc2n1] | 337.0; ¹H NMR (400 MHz, DMSO- d₆) δ 9.38 (d, J = 2.4 Hz, 1H), 8.91 (d, J = 2.4 Hz, 1H), 8.53 (s, 1H), 8.24 (d, J = 8.4 Hz, 1H), 8.08 (d, J = 8.4 Hz, 1H), 7.4 (s, 1H), 6.24 (s, 1H), 4.24 (s, 3H), 2.49-2.35 (m, 4H), 1.94-1.90 (m, 1H), 1.82- 1.77 (m, 1H) | |
| | 1-(6-(2-methyl-2H-pyrazolo[3,4- | | |
| | b]pyridin-5-yl)thieno[2,3- | | |
| | b]pyridin-2-yl)cyclobutanol | | |
| C22 | 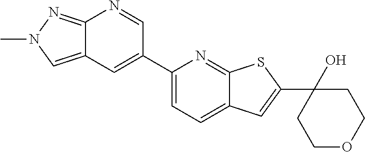 [SMILES: Cn1cc2cc(-c3ccc4cc(C5(O)CCOCC5)sc4n3)cnc2n1] | 367.0; ¹H NMR (400 MHz, DMSO- d₆) δ 9.42-9.38 (m, , 1H), 8.90- 8.95 (m, 1H), 8.54 (s, 1H), 8.25 (d, J = 8.4 Hz, 1H), 8.09 (d, J = 8.4 Hz, 1H), 7.32 (s, 1H), 5.89 (s, 1H), 4.24 (s, 3H), 3.78-3.76 (m, 4H), 2.11- 2.05 (m, 2H), 1.82 (br d, J = 12.0 Hz, 2H) | |
| | 4-(6-(2-methyl-2H-pyrazolo[3,4- | | |
| | b]pyridin-5-yl)thieno[2,3- | | |
| | b]pyridin-2-yl)tetrahydro-2H- | | |
| | pyran-4-ol | | |
| C25 | 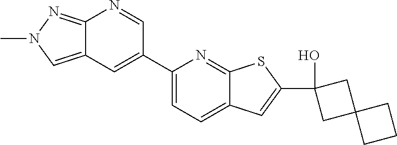 [SMILES: Cn1cc2cc(-c3ccc4cc(C5(O)CC6(CCC6)C5)sc4n3)cnc2n1] | 377.1; ¹H NMR (400 MHz, DMSO- d₆) δ 9.38 (d, J = 2.3 Hz, 1H), 8.91 (d, J = 2.3 Hz, 1H), 8.54 (s, 1H), 8.25 (d, J = 8.4 Hz, 1H), 8.08 (d, J = 8.4 Hz, 1H), 7.30 (s, 1H), 6.10 (s, 1H), 4.24 (s, 3H), 2.64 (d, J = 12.5 Hz, 2H), 2.45 (d, J = 12.7 Hz, 2H), 2.10 (t, J = 7.4 Hz, 2H), 2.02 (t, J = 7.5 Hz, 2H), 1.87-1.75 (m, 2H) | |
| | 2-(6-(2-methyl-2HI-pyrazolo[3,4- | | |
| | b]pyridin-5-yl)thieno[2,3- | | |
| | b]pyridin-2-yl)spiro[3.3]heptan- | | |
| | 2-ol | | |
| C26 | 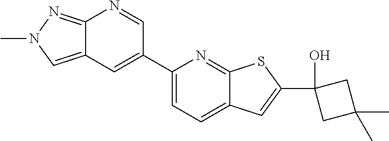 | 365.1; ¹H NMR (401 MHz, DMSO- d₆) δ 9.38 (d, J = 2.3 Hz, 1H), 8.91 (d, J = 2.3 Hz, 1H), 8.54 (s, 1H), 8.26 (d, J = 8.4 Hz, 1H), 8.09 (d, J = 8.4 Hz, 1H), 7.35 (s, 1H), 6.10 (s, 1H), 4.24 (s, 3H), 2.47-2.41 (m, 2H), 2.28-2.18 (m, 2H), 1.32 (s, 3H), 1.16 (s, 3H) | |
| | 3,3-dimethyl-1-(6-(2-methyl-2H- | | |
| | pyrazolo[3,4-b]pyridin-5- | | |
| | yl)thieno[2,3-b]pyridin-2- | | |
| | yl)cyclobutanol | | |
| C27 | 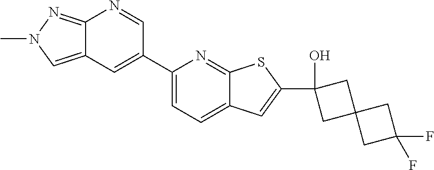 [SMILES: Cn1cc2cc(-c3ccc4cc(C5(O)CC6(CC(F)(F)C6)C5)sc4n3)cnc2n1] | 413.2; ¹H NMR (401 MHz, DMSO- d₆) δ 9.38 (d, J = 2.4 Hz, 1H), 8.91 (d, J = 2.3 Hz, 1H), 8.55 (s, 1H), 8.25 (d, J = 8.3 Hz, 1H), 8.09 (d, J = 8.3 Hz, 1H), 7.38 (s, 1H), 6.29 (s, 1H), 4.24 (s, 3H), 2.80-2.62 (m, 6H), 2.59 (d, J = 12.7 Hz, 2H) | |
| | 6,6-difluoro-2-(6-(2-methyl-2H- | | |
| | pyrazolo[3,4-b]pyridin-5- | | |
| | yl)thieno[2,3-b]pyridin-2- | | |
| | yl)spiro[3.3]heptan-2-ol | | |
| C28 | 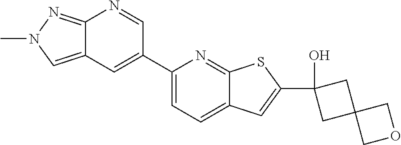 [SMILES: Cn1cc2cc(-c3ccc4cc(C5(O)CC6(COC6)C5)sc4n3)cnc2n1] | 379.2; ¹H NMR (401 MHz, DMSO- d₆) δ 9.37 (d, J = 2.3 Hz, 1H), 8.91 (d, J = 2.3 Hz, 1H), 8.54 (s, 1H), 8.24 (d, J = 8.4 Hz, 1H), 8.09 (d, J = 8.4 Hz, 1H), 7.30 (s, 1H), 6.25 (s, 1H), 4.64 (d, J = 17.1 Hz, 4H), 4.24 (s, 3H), 2.86-2.79 (m, 2H), 2.65- 2.57 (m, 2H) | |
| | 6-(6-(2-methyl-2H-pyrazolo[3,4- | | |
| | b]pyridin-5-yl)thieno[2,3- | | |
| | b]pyridin-2-yl)-2- | | |
| | oxaspiro[3.3]heptan-6-ol | | |
| C29C | 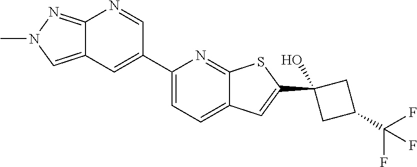 [SMILES: Cn1cc2cc(-c3ccc4cc([C@]5(O)C[C@@H](C(F)(F)F)C5)sc4n3)cnc2n1] | 405.2; ¹H NMR (400 MHz, DMSO- d₆) δ 9.39 (d, J = 2.3 Hz, 1H), 8.93 (d, J = 2.3 Hz, 1H), 8.55 (s, 1H), 8.26 (d, J = 8.4 Hz, 1H), 8.12 (d, J = 8.4 Hz, 1H), 7.56 (s, 1H), 6.68 (s, 1H), 4.25 (s, 3H), 3.09 (h, J = 8.8 Hz, 1H), 2.80-2.69 (m, 2H), 2.59- 2.55 (m, 2H) | |
| | cis-1-(6-(2-methyl-2H- | | |
| | pyrazolo[3,4-b]pyridin-5- | | |
| | yl)thieno[2,3-b]pyridin-2-yl)-3- | | |
| | (trifluoromethyl)cyclobutanol | | |
| C31C | 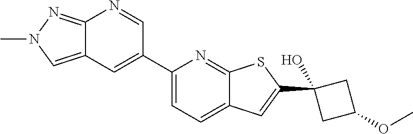 | 367.2; ¹H NMR (401 MHz, DMSO- d₆) δ 9.38 (d, J = 2.3 Hz, 1H), 8.92 (d, J = 2.3 Hz, 1H), 8.55 (s, 1H), 8.24 (d, J = 8.4 Hz, 1H), 8.09 (d, J = 8.4 Hz, 1H), 7.36 (s, 1H), 6.39 (s, 1H), 4.25 (s, 3H), 3.81 (p, J = 7.1 Hz, 1H), 3.20 (s, 3H), 2.87 (ddd, J = 9.7, 6.9, 3.0 Hz, 2H), 2.41-2.34 (m, 2H) | |
| | cis-3-methoxy-1-(6-(2-methyl- | | |
| | 2H-pyrazolo[3,4-b]pyridin-5- | | |
| | yl)thieno[2,3-b]pyridin-2- | | |
| | yl)cyclobutanol | | |
| C40C | 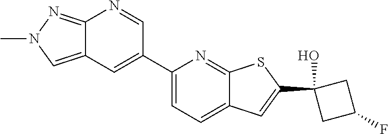 [SMILES: Cn1cc2cc(-c3ccc4cc([C@]5(O)C[C@@H](F)C5)sc4n3)cnc2n1] | 355.0; ¹H NMR (401 MHz, DMSO- d₆) δ 9.37 (d, J = 2.2 Hz, 1H), 8.91 (d, J = 2.2 Hz, 1H), 8.54 (s, 1H), 8.23 (d, J = 8.4 Hz, 1H), 8.09 (d, J = 8.4 Hz, 1H), 7.37 (s, 1H), 6.61 (s, 1H), 5.28-4.83 (m, 1H), 4.25 (s, 3H), 3.14-2.96 (m, 2H), 2.81- 2.60 (m, 2H) | Alternate conditions (5) and (7) were used |
| | cis-3-fluoro-1-(6-(2-methyl-2H- | | |
| | pyrazolo[3,4-b]pyridin-5- | | |
| | yl)thieno[2,3-b]pyridin-2- | | |
| | yl)cyclobutanol | | |
| C52 | 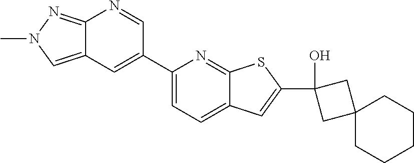 [SMILES: Cn1cc2cc(-c3ccc4cc(C5(O)CC6(CCCCC6)C5)sc4n3)cnc2n1] | 405.2; ¹H NMR (400 MHz, DMSO- d₆) δ 9.38 (d, J = 2.3 Hz, 1H), 8.91 (d, J = 2.4 Hz, 1H), 8.55 (s, 1H), 8.26 (d, J = 8.4 Hz, 1H), 8.09 (d, J = 8.4 Hz, 1H), 7.33 (s, 1H), 6.11 (s, 1H), 4.24 (s, 3H), 2.36 (d, J = 12.9 Hz, 2H), 2.25-2.13 (m, 2H), 1.66 (d, J = 6.6 Hz, 2H), 1.55-1.27 (m, 8H) | |
| | 2-(6-(2-methyl-2H-pyrazolo[3,4- | | |
| | b]pyridin-5-yl)thieno[2,3- | | |
| | b]pyridin-2-yl)spiro[3.5]nonan- | | |
| | 2-ol | | |
| C53 | 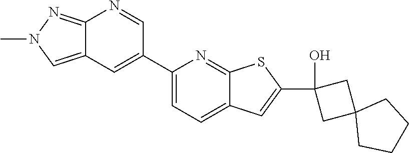 [SMILES: Cn1cc2cc(-c3ccc4cc(C5(O)CC6(CCCC6)C5)sc4n3)cnc2n1] | 391.2; ¹H NMR (400 MHz, DMSO- d₆) δ 9.47-9.32 (m, 1H), 8.91 (dd, J = 2.4, 1.0 Hz, 1H), 8.54 (s, 1H), 8.26 (dd, J = 8.5, 1.0 Hz, 1H), 8.09 (d, J = 8.2 Hz, 1H), 7.36 (d, J = 1.0 Hz, 1H), 6.11 (d, J = 1.1 Hz, 1H), 4.24 (s, 3H), 2.54 (s, 2H), 2.35 (d, J = 12.4 Hz, 2H), 1.77 (t, J = 6.8 Hz, 2H), 1.70-1.46 (m, 6H) | Alternate condition (5) was used |
| | 2-(6-(2-methyl-2H-pyrazolo[3,4- | | |
| | b]pyridin-5-yl)thieno[2,3- | | |
| | b]pyridin-2-yl)spiro[3.4]octan-2- | | |
| | ol | | |
| C56T | 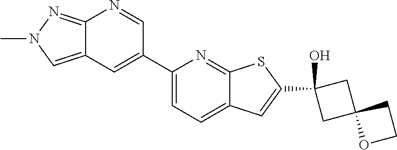 [SMILES: Cn1cc2cc(-c3ccc4cc([C@]5(O)C[C@@]6(CCO6)C5)sc4n3)cnc2n1] | 379.2; ¹H NMR (401 MHz, DMSO- d₆) δ 9.38 (d, J = 2.4 Hz, 1H), 8.91 (d, J = 2.4 Hz, 1H), 8.54 (s, 1H), 8.25 (d, J = 8.4 Hz, 1H), 8.09 (d, J = 8.5 Hz, 1H), 7.31 (s, 1H), 6.25 (s, 1H), 4.47 (t, J = 7.5 Hz, 2H), 4.24 (s, 3H), 2.82 (t, J = 7.5 Hz, 2H), 2.79-2.67 (m, 4H). | Alternate conditions (5) and (7) were used; SFC was performed after Step 1; Peak 1/SFC: Chiralpak AD-H 150 × 50 mm, 5 μm; Mobile phase: 50% (1:1) MeOH:ACN in CO₂ with a flow rate of 150 mL/min |
| | trans-6-(6-(2-methyl-2H- | | |
| | pyrazolo[3,4-b]pyridin-5- | | |
| | yl)thieno[2,3-b]pyridin-2-yl)-1- | | |
| | oxaspiro[3.3]heptan-6-ol | | |
| C56C | 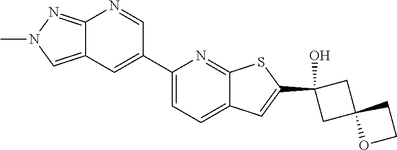 [SMILES: Cn1cc2cc(-c3ccc4cc([C@]5(O)C[C@]6(CCO6)C5)sc4n3)cnc2n1] | 379.2; ¹H NMR (401 MHz, DMSO- d₆) δ 9.38 (t, J = 2.1 Hz, 1H), 8.91 (t, J = 2.1 Hz, 1H), 8.55 (d, J = 1.9 Hz, 1H), 8.25 (dd, J = 8.4, 1.9 Hz, 1H), 8.09 (dd, J = 8.4, 1.9 Hz, 1H), 7.31 (d, J = 1.9 Hz, 1H), 6.32 (s, 1H), 4.41 (td, J = 7.5, 1.9 Hz, 2H), 4.25 (d, J = 1.8 Hz, 3H), 3.06-2.97 (m, 2H), 2.69 (q, J = 7.2 Hz, 4H). | Alternate conditions (5) and (7) were used; SFC was performed after Step 1; Peak 2/SFC: Chiralpak AD-H 150 × 50 mm, 5 μm; Mobile phase: 50% (1:1) MeOH:ACN in CO₂ with a flow rate of 150 mL/min |
| | cis-6-(6-(2-methyl-2H- | | |
| | pyrazolo[3,4-b]pyridin-5- | | |
| | yl)thieno[2,3-b]pyridin-2-yl)-1- | | |
| | oxaspiro[3.3]heptan-6-ol | | |
| C57 | 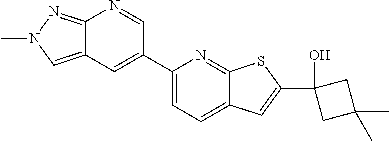 | 366.2; ¹H NMR (401 MHz, DMSO- d₆) δ 9.63 (d, J = 2.2 Hz, 1H), 9.30 (s, 1H), 9.22 (d, J = 2.2 Hz, 1H), 8.59 (s, 1H), 7.48 (s, 1H), 6.28 (s, 1H), 4.25 (s, 3H), 2.48-2.40 (m, 2H), 2.30-2.23 (m, 2H), 1.33 (s, 3H), 1.17 (s, 3H) | Alternate condition (5) was used |
| | 3,3-dimethyl-1-(2-(2-methyl-2H- | | |
| | pyrazolo[3,4-b]pyridin-5- | | |
| | yl)thieno[2,3-d]pyrimidin-6- | | |
| | yl)cyclobutanol | | |
| C58C | 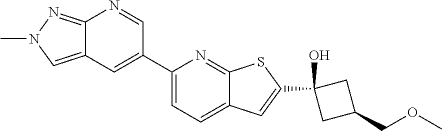 | 381.2; ¹H NMR (400 MHz, DMSO- d₆) δ 9.38 (d, J = 2.3 Hz, 1H), 8.92 (d, J = 2.3 Hz, 1H), 8.54 (s, 1H), 8.25 (d, J = 8.4 Hz, 1H), 8.09 (d, J = 8.4 Hz, 1H), 7.42 (s, 1H), 6.25 (s, 1H), 4.24 (s, 3H), 3.43 (d, J = 6.2 Hz, 2H), 3.28 (s, 3H), 2.58 (td, J = 7.6, 3.9 Hz, 2H), 2.40-2.31 (m, 1H), 2.20 (td, J = 9.2, 2.6 Hz, 2H) | Alternate condition (5) was used; SFC was performed after Step 1; Peak 2/SFC: Chiralpak AD-H 150 × 50 mm, 5 μm; Mobile phase: 50% (1:1) MeOH:ACN in CO₂ with a flow rate of 150 mL/min |
| | cis-3-(methoxymethyl)-1-(6-(2- | | |
| | methyl-2H-pyrazolo[3,4- | | |
| | b]pyridin-5-yl)thieno[2,3- | | |
| | b]pyridin-2-yl)cyclobutanol | | |
| C59T | 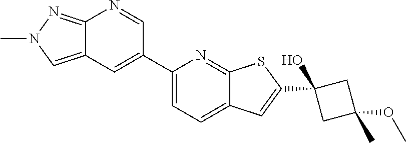 | 381.2; ¹H NMR (400 MHz, DMSO- d₆) δ 9.38 (d, J = 2.3 Hz, 1H), 8.91 (d, J = 2.3 Hz, 1H), 8.55 (s, 1H), 8.27 (d, J = 8.4 Hz, 1H), 8.09 (d, J = 8.4 Hz, 1H), 7.32 (s, 1H), 6.26 (s, 1H), 4.24 (s, 3H), 3.13 (s, 3H), 2.62 (d, J = 13.2 Hz, 2H), 2.35 (d, J = 13.4 Hz, 2H), 1.50 (s, 3H) | Alternate conditions (5) and (7) were used; First eluting isomer after step (1): purification by silica gel chromatography (eluent of 15-30% EtOAc in hexanes) |
| | trans-3-methoxy-3-methyl-1-(6- | | |
| | (2-methyl-2H-pyrazolo[3,4- | | |
| | b]pyridin-5-yl)thieno[2,3- | | |
| | b]pyridin-2-yl)cyclobutanol | | |
| C61 | 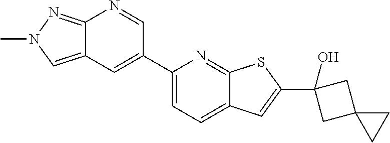 [SMILES: Cn1cc2cc(-c3ccc4cc(C5(O)CC6(CC6)C5)sc4n3)cnc2n1] | 363.2; ¹H NMR (400 MHz, DMSO- d₆) δ 9.39 (d, J = 2.3 Hz, 1H), 8.92 (d, J = 2.3 Hz, 1H), 8.55 (d, J = 0.6 Hz, 1H), 8.28 (d, J = 8.4 Hz, 1H), 8.10 (d, J = 8.4 Hz, 1H), 7.45 (s, 1H), 6.39 (s, 1H), 4.25 (s, 3H), 2.79 -2.66 (m, 2H), 2.50-2.45 (m, 2H), 0.71-0.40 (m, 4H) | Alternate condition (5) was used |
| | 5-(6-(2-methyl-2H-pyrazolo[3,4- | | |
| | b]pyridin-5-yl)thieno[2,3- | | |
| | b]pyridin-2-yl)spiro[2.3]hexan- | | |
| | 5-o1 | | |
| C62C | 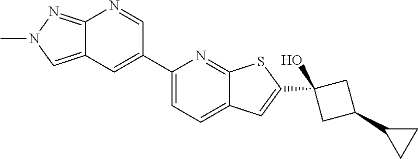 [SMILES: Cn1cc2cc(-c3ccc4cc([C@]5(O)C[C@@H](C6CC6)C5)sc4n3)cnc2n1] | 377.2; ¹H NMR (400 MHz, DMSO- d₆) δ 9.38 (d, J = 2.3 Hz, 1H), 8.91 (d, J = 2.3 Hz, 1H), 8.54 (s, 1H), 8.24 (d, J = 8.4 Hz, 1H), 8.08 (d, J = 8.4 Hz, 1H), 7.38 (s, 1H), 6.20 (s, 1H), 4.24 (s, 3H), 2.64-2.52 (m, 2H), 2.11 (td, J = 9.5, 2.8 Hz, 2H), 1.80 (p, J = 8.4 Hz, 1H), 0.99-0.82 (m, 1H), 0.40 (dt, J = 8.3, 2.9 Hz, 2H), 0.14 (q, J = 5.0 Hz, 2H) | Alternate condition (5) was used; Peak 2/SFC: Chiralpak AS- H(250 × 30) mm, 5 μm; Mobile phase: 30% (1:1) MeOH:ACN in CO₂ with a flow rate of 150 mL/min |
| | cis-3-cyclopropyl-1-(6-(2- | | |
| | methyl-2H-pyrazolo[3,4- | | |
| | b]pyridin-5-yl)thieno[2,3- | | |
| | b]pyridin-2-yl)cyclobutanol | | |
| C64SR | 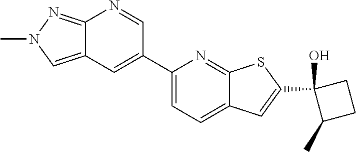 [SMILES: C[C@@H]1CC[C@@]1(O)c1cc2ccc(-c3cnc4nn(C)cc4c3)nc2s1] | 351.2; ¹H NMR (400 MHz, DMSO- d₆) δ 9.38 (d, J = 2.3 Hz, 1H), 8.91 (d, J = 2.4 Hz, 1H), 8.54 (s, 1H), 8.25 (d, J = 8.4 Hz, 1H), 8.09 (d, J = 8.4 Hz, 1H), 7.35 (s, 1H), 5.95 (s, 1H), 4.24 (s, 3H), 2.75-2.62 (m, 1H), 2.47-2.40 (m, 1H), 2.28 (ddd, J = 11.7, 8.0, 4.6 Hz, 1H), 2.12- 1.92 (m, 1H), 1.67 (ddt, J = 9.3, 7.4, 5.1 Hz, 1H), 1.10 (d, J = 7.0 Hz, 3H) | Alternate condition (5) was used; SFC was performed after Step 1; Peak 1/SFC: Chiralpak IG 250 × 50 mm, 5um; Mobile phase: 40% (1:1) IPA:ACN in CO₂ with a flow rate of 180 mL/min |
| | (1S,2R)-2-methyl-1-(6-(2- | | |
| | methyl-2H-pyrazolo[3,4- | | |
| | b]pyridin-5-yl)thieno[2,3- | | |
| | b]pyridin-2-yl)cyclobutanol | | |
| C64RS | 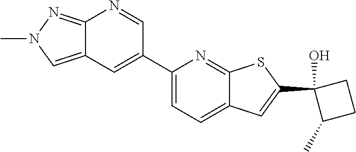 [SMILES: C[C@H]1CC[C@]1(O)c1cc2ccc(-c3cnc4nn(C)cc4c3)nc2s1] | 351.2; ¹H NMR (400 MHz, DMSO- d₆) δ 9.38 (d, J = 2.3 Hz, 1H), 8.92 (d, J = 2.3 Hz, 1H), 8.55 (s, 1H), 8.25 (d, J = 8.4 Hz, 1H), 8.09 (d, J = 8.4 Hz, 1H), 7.36 (s, 1H), 5.97 (s, 1H), 4.24 (s, 3H), 2.68 (q, J = 7.5 Hz, 1H), 2.49-2.39 (m, 1H), 2.28 (ddd, J = 11.8, 9.3, 5.0 Hz, 1H), 2.09-1.97 (m, 1H), 1.67 (ddt, J = 10.7, 9.2, 7.4 Hz, 1H), 1.10 (d, J = 7.0 Hz, 3H) | Alternate condition (5) was used; SFC was performed after Step 1; Peak 2/SFC: Chiralpak IG 250 × 50 mm, 5 μm; Mobile phase: 40% (1:1) IPA:ACN in CO₂ with a flow rate of 180 mL/min |
| | (1R,2S)-2-methyl-1-(6-(2- | | |
| | methyl-2H-pyrazolo[3,4- | | |
| | b]pyridin-5-yl)thieno[2,3- | | |
| | b]pyridin-2-yl)cyclobutanol | | |
| C65S | 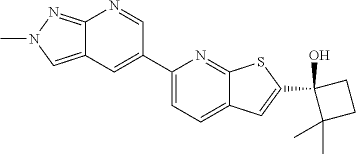 [SMILES: Cn1cc2cc(-c3ccc4cc([C@]5(O)CCC5(C)C)sc4n3)cnc2n1] | 365.2; ¹H NMR (400 MHz, DMSO- d₆) δ 9.34 (d, J = 2.3 Hz, 1H), 8.87 (d, J = 2.3 Hz, 1H), 8.50 (s, 1H), 8.26 (d, J = 8.4 Hz, 1H), 8.03 (d, J = 8.4 Hz, 1H), 7.33 (s, 1H),6.86 (s, 1H), 4.21 (s, 3H), 2.73-2.62 (m, 1H), 2.20 (ddd, J = 12.1, 9.6, 6.7 Hz, 1H), 1.80 (td, J = 10.1, 5.9 Hz, 1H), 1.61 (ddd, J = 10.8, 8.9, 6.7 Hz, 1H), 1.16 (s, 3H), 0.74 (s, 3H) | Alternate condition (5) was used; SFC was performed after Step 1; Peak 1/SFC: Chiralpak AD-H 250 × 4.6 mm, 5 μm; Mobile phase: 50% 1:1 (MeOH: 0.2% NH₄OH in ACN) in CO₂ with a flow rate of 5 mL/min |
| | (1S)-2,2-dimethyl-1-(6-(2- | | |
| | methyl-2H-pyrazolo[3,4- | | |
| | b]pyridin-5-yl)thieno[2,3- | | |
| | b]pyridin-2-yl)cyclobutanol | | |
| C65R | 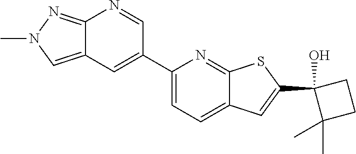 [SMILES: Cn1cc2cc(-c3ccc4cc([C@@]5(O)CCC5(C)C)sc4n3)cnc2n1] | 365.2; ¹H NMR (400 MHz, DMSO- d₆) δ 9.34 (d, J = 2.3 Hz, 1H), 8.87 (d, J = 2.3 Hz, 1H), 8.50 (s, 1H), 8.26 (d, J = 8.4 Hz, 1H), 8.03 (d, J = 8.4 Hz, 1H), 7.33 (s, 1H),6.86 (s, 1H), 4.21 (s, 3H), 2.73-2.62 (m, 1H), 2.20 (ddd, J = 12.1, 9.6, 6.7 Hz, 1H), 1.80 (td, J = 10.1, 5.9 Hz, 1H), 1.61 (ddd, J = 10.8, 8.9, 6.7 Hz, 1H), 1.16 (s, 3H), 0.74 (s, 3H) | Alternate condition (5) was used; SFC was performed after Step 1; Peak 2/SFC: Chiralpak AD-H 250 × 4.6 mm, 5 μm; Mobile phase: 50% 1:1 (MeOH: 0.2% NH₄OH in ACN) in CO₂ with a flow rate of 5 mL/min |
| | (1R)-2,2-dimethyl-1-(6-(2- | | |
| | methyl-2H-pyrazolo[3,4- | | |
| | b]pyridin-5-yl)thieno[2,3- | | |
| | b]pyridin-2-yl)cyclobutanol | | |
| C66T | 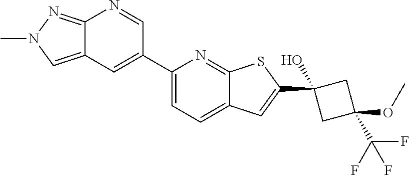 [SMILES: CO[C@]1(C(F)(F)F)C[C@](O)(c2cc3ccc(-c4cnc5nn(C)cc5c4)nc3s2)C1] | 435.2; ¹H NMR (400 MHz, DMSO- d₆) δ 9.39 (d, J = 2.3 Hz, 1H), 8.93 (d, J = 2.4 Hz, 1H), 8.56 (s, 1H), 8.31 (d, J = 8.4 Hz, 1H), 8.12 (d, J = 8.5 Hz, 1H), 7.45 (s, 1H), 6.69 (s, 1H), 4.25 (s, 3H), 3.36 (d, J = 1.5 Hz, 3H), 2.91 (d, J = 14.3 Hz, 2H), 2.79-2.69 (m, 2H) | Alternate conditions (5) and (7) were used: First eluting isomer after step (1): purification by silica gel chromatography (eluent of 5-20% EtOAc in hexanes) |
| | trans-3-methoxy-1-(6-(2-methyl- | | |
| | 2H-pyrazolo[3,4-b]pyridin-5- | | |
| | yl)thieno[2,3-b]pyridin-2-yl)-3- | | |
| | (trifluoromethyl)cyclobutanol | | |
| C66C | 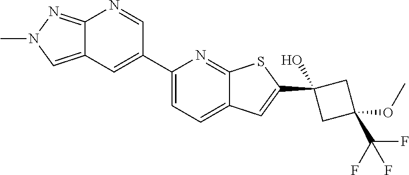 [SMILES: CO[C@]1(C(F)(F)F)C[C@@](O)(c2cc3ccc(-c4cnc5nn(C)cc5c4)nc3s2)C1] | 435.2; ¹H NMR (400 MHz, DMSO- d₆) δ 9.38 (d, J = 2.3 Hz, 1H), 8.93 (d, J = 2.3 Hz, 1H), 8.55 (s, 1H), 8.29 (d, J = 8.4 Hz, 1H), 8.11 (d, J = 8.4 Hz, 1H), 7.41 (s, 1H), 6.68 (s, 1H), 4.25 (s, 3H), 3.46 (s, 3H), 3.08- 2.92 (m, 2H), 2.76 (d, J = 14.5 Hz, 2H) | Alternate conditions (5) and (7) were used; Second eluting isomer after step (1): purification by silica gel chromatography (eluent of 5-20% EtOAc in hexanes) |
| | cis-3-methoxy-1-(6-(2-methyl- | | |
| | 2H-pyrazolo[3,4-b]pyridin-5- | | |
| | yl)thieno[2,3-b]pyridin-2-yl)-3- | | |
| | (trifluoromethyl)cyclobutanol | | |
| C68 | 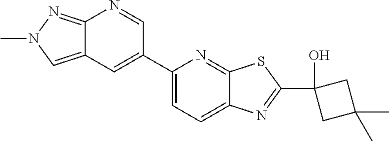 | 366.2; ¹H NMR (400 MHz, DMSO- d₆) δ 9.39 (d, J = 2.3 Hz, 1H), 8.95 (d, J = 2.3 Hz, 1H), 8.56 (s, 1H), 8.47 (d, J = 8.6 Hz, 1H), 8.24 (d, J = 8.7 Hz, 1H), 6.76 (s, 1H), 4.25 (s, 3H), 2.65-2.55 (m, 2H), 2.27- 2.15 (m, 2H), 1.35 (s, 3H), 1.26 (s, 3H) | Alternate condition (5) was used |
| | 3,3-dimethyl-1-(5-(2-methyl-2H- | | |
| | pyrazolo[3,4-b]pyridin-5- | | |
| | yl)[1,3]thiazolo[5,4-b]pyridin-2- | | |
| | yl)cyclobutanol | | |
| C70SR | 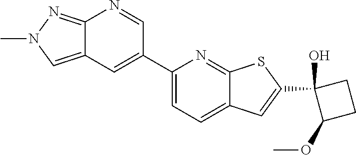 [SMILES: CO[C@@H]1CC[C@@]1(O)c1cc2ccc(-c3cnc4nn(C)cc4c3)nc2s1] | 367.2; ¹H NMR (400 MHz, DMSO- d₆) δ 9.38 (d, J = 2.3 Hz, 1H), 8.91 (d, J = 2.3 Hz, 1H), 8.54 (s, 1H), 8.25 (d, J = 8.5 Hz, 1H), 8.09 (d, J = 8.4 Hz, 1H), 7.39 (s, 1H), 6.12 (s, 1H), 4.24 (s, 3H), 4.07-3.96 (m, 1H), 3.23 (s, 3H), 2.23-2.00 (m, 4H) | Alternate condition (5) was used; First eluting isomer after step (1): purification by silica gel chromatography (eluent of 15% EtOAc in pet. ether); Peak was then purified by SFC; Peak 1/SFC: Chiralpak IC 150 × 50 mm, 5 μm; Mobile phase: 40% MeOH in CO₂ using a flow rate of 120 mL/min |
| | (1S,2R)-2-methoxy-1-(6-(2- | | |
| | methyl-2H-pyrazolo[3,4- | | |
| | b]pyridin-5-yl)thieno[2,3- | | |
| | b]pyridin-2-yl)cyclobutanol | | |
| C70RS | 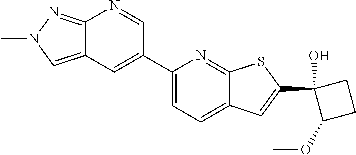 [SMILES: CO[C@H]1CC[C@]1(O)c1cc2ccc(-c3cnc4nn(C)cc4c3)nc2s1] | 367.2; ¹H NMR (400 MHz, DMSO- d₆) δ 9.38 (d, J = 2.3 Hz, 1H), 8.91 (d, J = 2.3 Hz, 1H), 8.55 (s, 1H), 8.25 (d, J = 8.3 Hz, 1H), 8.09 (d, J = 8.5 Hz, 1H), 7.39 (s, 1H), 6.11 (s, 1H), 4.24 (s, 3H), 4.06-3.95 (m, 1H), 3.23 (s, 3H), 2.21-2.01 (m, 4H) | Alternate condition (5) was used; First eluting isomer after step (1): purification by silica gel chromatography (eluent of 15% EtOAc in pet. ether); Peak was then purified by SFC; Peak 2/SFC: Chiralpak IC 150 × 50 mm, 5 μm; Mobile phase: 40% MeOH in CO₂ using a flow rate of 120 mL/min |
| | (1R,2S)-2-methoxy-1-(6-(2- | | |
| | methyl-2H-pyrazolo[3,4- | | |
| | b]pyridin-5-yl)thieno[2,3- | | |
| | b]pyridin-2-yl)cyclobutanol | | |
| C70RR | 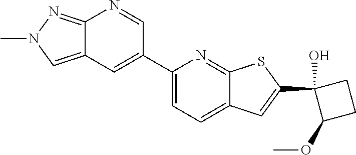 [SMILES: CO[C@@H]1CC[C@]1(O)c1cc2ccc(-c3cnc4nn(C)cc4c3)nc2s1] | 367.2; ¹H NMR (400 MHz, DMSO- d₆) δ 9.39 (d, J = 2.3 Hz, 1H), 8.92 (d, J = 2.3 Hz, 1H), 8.55 (s, 1H), 8.27 (d, J = 8.5 Hz, 1H), 8.09 (d, J = 8.4 Hz, 1H), 7.44 (s, 1H), 6.51 (s, 1H), 4.25 (s, 3H), 4.02 (t, J = 8.5 Hz, 1H), 3.20 (s, 3H), 2.37 (d, J = 10.9 Hz, 1H), 2.17 (q, J = 9.6 Hz, 1H), 1.94 (dd, J = 11.5, 9.4 Hz, 1H), 1.71 (q, J = 10.3 Hz, 1H) | Alternate condition (5) was used; Second eluting isomer after step (1): purification by silica gel chromatography (eluent of 15% EtOAc in pet. ether); Peak was then purified by SFC; Peak 2/SFC: Chiralpak IC 150 × 50 mm, 5 μm; Mobilephase: 40% MeOH in CO₂ using a flow rate of 120 mL/min |
| | (1R,2R)-2-methoxy-1-(6-(2- | | |
| | methyl-2H-pyrazolo[3,4- | | |
| | b]pyridin-5-yl)thieno[2,3- | | |
| | b]pyridin-2-yl)cyclobutanol | | |
| C70SS | 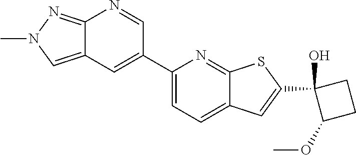 [SMILES: CO[C@H]1CC[C@@]1(O)c1cc2ccc(-c3cnc4nn(C)cc4c3)nc2s1] | 367.2; ¹H NMR (400 MHz, DMSO- d₆) δ 9.39 (d, J = 2.3 Hz, 1H), 8.92 (d, J = 2.3 Hz, 1H), 8.55 (s, 1H), 8.27 (d, J = 8.4 Hz, 1H), 8.09 (d, J = 8.4 Hz, 1H), 7.44 (s, 1H), 6.51 (s, 1H), 4.25 (s, 3H), 4.02 (t, J = 8.5 Hz, 1H), 3.20 (s, 3H), 2.37 (d, J = 10.9 Hz, 1H), 2.24-2.10 (m, 1H), 2.02-1.89 (m, 1H), 1.70 (p, J = 10.4 Hz, 1H) | Alternate condition (5) was used; Second eluting isomer after step (1): purification by silica gel chromatography (eluent of 15% EtOAc in pet. ether); Peak was then purified by SFC; Peak 1/SFC: Chiralpak IC 150 × 50 mm, 5 μm; Mobile phase: 40% MeOH in CO₂ using a flow rate of 120 mL/min |
| | (1S,2S)-2-methoxy-1-(6-(2- | | |
| | methyl-2H-pyrazolo[3,4- | | |
| | b]pyridin-5-yl)thieno[2,3- | | |
| | b]pyridin-2-yl)cyclobutanol | | |
| C76C | 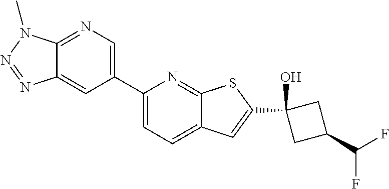 [SMILES: Cn1nnc2cc(-c3ccc4cc([C@]5(O)C[C@@H](C(F)F)C5)sc4n3)cnc21] | 388.0; ¹H NMR (401 MHz, DMSO- d₆) δ 9.58 (d, J = 2.0 Hz, 1H), 9.22 (d, J = 2.0 Hz, 1H), 8.33 (d, J = 8.4 Hz, 1H), 8.25 (d, J = 8.4 Hz, 1H), 7.54 (s, 1H), 6.55 (br s, 1H), 6.18 (td, J = 57.4, 3.6 Hz, 1H), 4.37 (s, 3H), 2.65-2.58 (m, 3H), 2.48- 2.41 (m, 2H) | |
| | cis-3-(difluoromethyl)-1-(6-(3- | | |
| | methyl-3H-[1,2,3]triazolo[4,5- | | |
| | b]pyridin-6-yl)thieno[2,3- | | |
| | b]pyridin-2-yl)cyclobutanol | | |
| C80C | 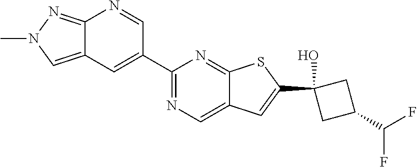 [SMILES: Cn1cc2cc(-c3ncc4cc([C@]5(O)C[C@@H](C(F)F)C5)sc4n3)cnc2n1] | 388.2; ¹H NMR (401 MHz, DMSO- d₆) δ 9.64 (d, J = 2.3 Hz, 1H), 9.29 (s, 1H), 9.23 (d, J = 2.2 Hz, 1H), 8.59 (s, 1H), 7.63 (s, 1H), 6.67 (s, 1H), 6.18 (td, J = 57.1, 3.6 Hz, 1H), 4.25 (s, 3H), 2.68-2.58 (m, 3H), 2.49-2.43 (m, 2H) | Alternate conditions (5) and (7) were used |
| | cis-3-(difluoromethyl)-1-(2-(2- | | |
| | methyl-2H-pyrazolo[3,4- | | |
| | b]pyridin-5-yl)thieno[2,3- | | |
| | d]pyrimidin-6-yl)cyclobutanol | | |
| C81C | 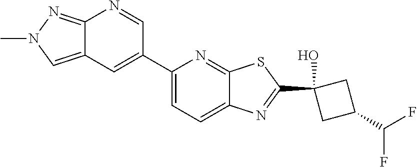 [SMILES: Cn1cc2cc(-c3ccc4nc([C@]5(O)C[C@@H](C(F)F)C5)sc4n3)cnc2n1] | 388.2; ¹H NMR (400 MHz, DMSO- d₆) δ 9.39 (d, J = 2.4 Hz, 1H), 8.96 (d, J = 2.3 Hz, 1H), 8.56 (s, 1H), 8.48 (d, J = 8.6 Hz, 1H), 8.26 (d, J = 8.7 Hz, 1H), 7.09 (s, 1H), 6.19 (td, J = 57.2, 4.3 Hz, 1H), 4.25 (s, 3H), 2.94-2.79 (m, 1H), 2.76- 2.66 (m, 2H), 2.48-2.39 (m, 2H) | Alternate condition (5) was used |
| | cis-3-(difluoromethyl)-1-(5-(2- | | |
| | methyl-2H-pyrazolo[3,4- | | |
| | b]pyridin-5-yl)[1,3]thiazolo[5,4- | | |
| | b]pyridin-2-yl)cyclobutanol | | |
| C82C | 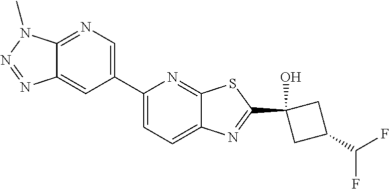 [SMILES: Cn1nnc2cc(-c3ccc4nc([C@]5(O)C[C@@H](C(F)F)C5)sc4n3)cnc21] | 389.2; ¹H NMR (401 MHz, DMSO- d₆) δ 9.58 (d, J = 2.0 Hz, 1H), 9.25 (d, J = 2.0 Hz, 1H), 8.54 (d, J = 8.6 Hz, 1H), 8.39 (d, J = 8.6 Hz, 1H), 7.13 (s, 1H), 6.20 (td, J = 57.2, 4.3 Hz, 1H), 4.37 (s, 3H), 2.86 (qd, J = 8.6, 4.0 Hz, 1H), 2.77- 2.68 (m, 2H), 2.49-2.40 (m, 2H) | Alternate condition (5) was used |
| | cis-3-(difluoromethyl)-1-(5-(3- | | |
| | methyl-3H-[1,2,3]triazolo[4,5- | | |
| | b]pyridin-6-yl)[1,3]thiazolo[5,4- | | |
| | b]pyridin-2-yl)cyclobutanol | | |
| C85C | 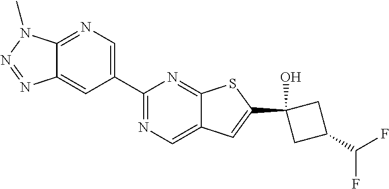 [SMILES: Cn1nnc2cc(-c3ncc4cc([C@]5(O)C[C@@H](C(F)F)C5)sc4n3)cnc21] | 389.1; ¹H NMR (401 MHz, DMSO- d₆) δ 9.82 (d, J = 1.9 Hz, 1H), 9.42- 9.35 (m, 2H), 7.69 (s, 1H), 6.70 (s, 1H), 6.18 (td, J = 56.9, 3.5 Hz, 1H), 4.38 (s, 3H), 2.70-2.50 (m, 5H) | Alternate conditions (5) and (7) were used |
| | cis-3-(difluoromethyl)-1-(2-(3- | | |
| | methyl-3H-[1,2,3]triazolo[4,5- | | |
| | b]pyridin-6-yl)thieno[2,3- | | |
| | d]pyrimidin-6-yl)cyclobutanol | | |
| C87C | 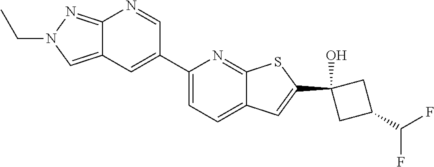 [SMILES: CCn1cc2cc(-c3ccc4cc([C@]5(O)C[C@@H](C(F)F)C5)sc4n3)cnc2n1] | 401.2; ¹H NMR (400 MHz, DMSO- d₆) δ 9.39 (d, J = 2.3 Hz, 1H), 8.92 (d, J = 2.4 Hz, 1H), 8.59 (s, 1H), 8.26 (d, J = 8.4 Hz, 1H), 8.11 (d, J = 8.4 Hz, 1H), 7.50 (s, 1H), 6.49 (s, 1H), 6.17 (td, J = 57.5, 3.7 Hz, 1H), 4.53 (q, J = 7.3 Hz, 2H), 2.68-2.54 (m, 3H), 2.44 (dd, J = 11.0, 6.3 Hz, 2H), 1.56 (t, J = 7.3 Hz, 3H). | Alternate condition (5) was used |
| | cis-3-(difluoromethyl)-1-(6-(2- | | |
| | ethyl-2H-pyrazolo[3,4- | | |
| | b]pyridin-5-yl)thieno[2,3- | | |
| | b]pyridin-2-yl)cyclobutanol | | |
| C88T | 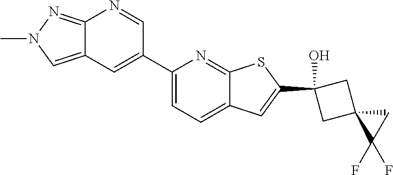 [SMILES: Cn1cc2cc(-c3ccc4cc([C@]5(O)C[C@@]6(CC6(F)F)C5)sc4n3)cnc2n1] | 399.2; ¹H NMR (401 MHz, DMSO- d₆) δ 9.39 (d, J = 2.3 Hz, 1H), 8.93 (d, J = 2.3 Hz, 1H), 8.55 (s, 1H), 8.33 (d, J = 8.4 Hz, 1H), 8.11 (d, J = 8.4 Hz, 1H), 7.35 (s, 1H), 6.72 (br s, 1H), 4.25 (s, 3H), 2.77-2.67 (m, 4H), 1.55 (t, J = 8.4 Hz, 2H). | Alternate conditions (5) and (7) were used; First eluting isomer after step (1): purification by silica gel chromatography (eluent of 15-20% EtOAC in pet. ether) |
| | trans-1,1-difluoro-5-(6-(2- | | |
| | methyl-2H-pyrazolo[3,4- | | |
| | b]pyridin-5-yl)thieno[2,3- | | |
| | b]pyridin-2-yl)spiro[2.3]hexan- | | |
| | 5-ol | | |
| C88C | 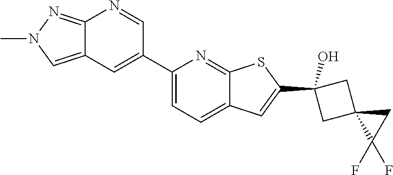 [SMILES: Cn1cc2cc(-c3ccc4cc([C@]5(O)C[C@]6(CC6(F)F)C5)sc4n3)cnc2n1] | 399.2; ¹H NMR (401 MHz, DMSO- d₆) δ 9.39 (d, J = 2.3 Hz, 1H), 8.93 (d, J = 2.3 Hz, 1H), 8.55 (s, 1H), 8.28 (d, J = 8.4 Hz, 1H), 8.11 (d, J = 8.4 Hz, 1H), 7.49 (s, 1H), 6.60 (s, 1H), 4.25 (s, 3H), 2.76-2.61 (m, 4H), 1.59 (t, J = 8.5 Hz, 2H) | Alternate conditions (5) and (7) were used; Second eluting isomer after step (1): purification by silica gel chromatography (eluent of 15-20% EtOAC in pet. ether) |
| | cis-1,1-difluoro-5-(6-(2-methyl- | | |
| | 2H-pyrazolo[3,4-b ]pyridin-5- | | |
| | yl)thieno[2,3-b]pyridin-2- | | |
| | yl)spiro[2.3]hexan-5-ol | | |
| C89C | 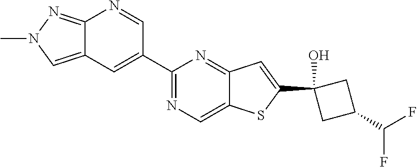 [SMILES: Cn1cc2cc(-c3ncc4sc([C@]5(O)C[C@@H](C(F)F)C5)cc4n3)cnc2n1] | 388.2; ¹H NMR (401 MHz, DMSO- d₆) δ 9.66 (d, J = 2.2 Hz, 1H), 9.53 (s, 1H), 9.21 (d, J = 2.3 Hz, 1H), 8.60 (s, 1H), 7.78 (s, 1H), 6.76 (s, 1H), 6.19 (td, J = 57.4, 3.6 Hz, 1H), 4.25 (s, 3H), 2.82-2.75 (m, 1H), 2.71-2.61 (m, 2H), 2.47-2.40 (m, 2H) | Alternate conditions (5) and (7) were used |
| | cis-3-(difluoromethyl)-1-(2-(2- | | |
| | methyl-2H-pyrazolo[3,4- | | |
| | b]pyridin-5-yl)thieno[3,2- | | |
| | d]pyrimidin-6-yl)cyclobutanol | | |
| C90C | 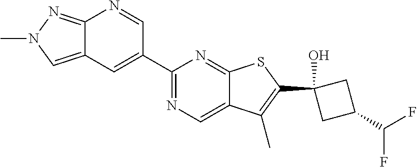 [SMILES: Cc1c2cnc(-c3cnc4nn(C)cc4c3)nc2sc1[C@]1(O)C[C@@H](C(F)F)C1] | 402.2; ¹H NMR (401 MHz, DMSO- d₆) δ 9.65 (d, J = 2.2 Hz, 1H), 9.33 (s, 1H), 9.23 (d, J = 2.2 Hz, 1H), 8.59 (s, 1H), 6.35 (s, 1H), 6.13 (td, J = 57.3, 4.4 Hz, 1H), 4.25 (s, 3H), 2.82-2.72 (m, 3H), 2.56 (s, 3H), 2.50-2.43 (m, 2H) | Alternate conditions (5) and (7) were used |
| | cis-3-(difluoromethyl)-1-(5- | | |
| | methyl-2-(2-methyl-2H- | | |
| | pyrazolo[3,4-b]pyridin-5- | | |
| | yl)thieno[2,3-d]pyrimidin-6- | | |
| | yl)cyclobutanol | | |
| C91T | 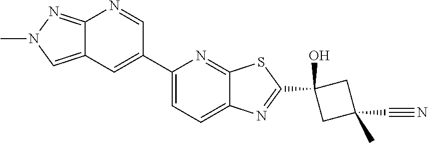 [SMILES: Cn1cc2cc(-c3ccc4nc([C@]5(O)C[C@](C)(C#N)C5)sc4n3)cnc2n1] | 377.2; ¹H NMR (400 MHz, DMSO- d₆) δ 9.40 (d, J = 2.3 Hz, 1H), 8.97 (d, J = 2.4 Hz, 1H), 8.57 (s, 1H), 8.53 (d, J = 8.6 Hz, 1H), 8.28 (d, J = 8.7 Hz, 1H), 7.23 (s, 1H), 4.25 (s, 3H), 3.30 (d, J = 10.8 Hz, 2H), 2.57 (d, J = 2.9 Hz, 2H), 1.73 (s, 3H) | Alternate condition (5) was used; Second eluting isomer after step (1): purification by silica gel chromatography (eluent of 15% EtOAC in pet. ether) |
| | trans-3-hydroxy-1-methyl-3-(5- | | |
| | (2-methyl-2H-pyrazolo[3,4- | | |
| | b]pyridin-5-yl)[1,3]thiazolo[5,4- | | |
| | b]pyridin-2- | | |
| | yl)cyclobutanecarbonitrile | | |
| C91C | 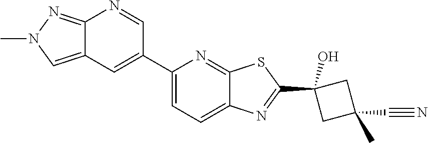 [SMILES: Cn1cc2cc(-c3ccc4nc([C@]5(O)C[C@@](C)(C#N)C5)sc4n3)cnc2n1] | 377.2; ¹H NMR (400 MHz, DMSO- d₆) δ 9.39 (d, J = 2.3 Hz, 1H), 8.96 (d, J = 2.4 Hz, 1H), 8.57 (s, 1H), 8.50 (d, J = 8.7 Hz, 1H), 8.27 (d, J = 8.7 Hz, 1H), 7.32 (s, 1H), 4.25 (s, 3H), 2.95-2.85 (m, 4H), 1.67 (s, 3H | Alternate condition (5) was used; First eluting isomer after step (1): purification by silica gel chromatography (eluent of 15% EtOAC in pet. ether) |
| | cis-3-hydroxy-1-methyl-3-(5-(2- | | |
| | methyl-2H-pyrazolo[3,4- | | |
| | b]pyridin-5-yl)[1,3]thiazolo[5,4- | | |
| | b]pyridin-2- | | |
| | yl)cyclobutanecarbonitrile | | |
| C92C | 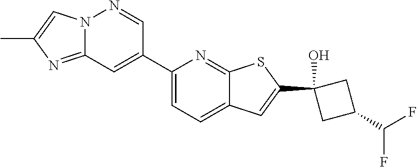 [SMILES: Cc1cn2ncc(-c3ccc4cc([C@]5(O)C[C@@H](C(F)F)C5)sc4n3)cc2n1] | 387.2; ¹H NMR (401 MHz, DMSO- d₆) δ 9.27 (d, J = 2.3 Hz, 1H), 8.69 (d, J = 2.1 Hz, 1H), 8.30 (d, J = 8.5 Hz, 1H), 8.25 (d, J = 8.4 Hz, 1H), 8.16 (s, 1H), 7.52 (s, 1H), 6.53 (s, 1H), 6.17 (td, J = 57.3, 3.6 Hz, 1H), 2.68-2.58 (m, 3H), 2.53-2.40 (m, 5H) | Alternate condition (5) was used |
| | cis-3-(difluoromethyl)-1-(6-(2- | | |
| | methylimidazo[1,2-b]pyridazin- | | |
| | 7-yl)thieno[2,3-b]pyridin-2- | | |
| | yl)cyclobutanol | | |
| C93C | 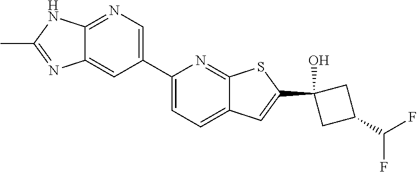 [SMILES: Cc1nc2cc(-c3ccc4cc([C@]5(O)C[C@@H](C(F)F)C5)sc4n3)cnc2[nH]1] | 387.2; ¹H NMR (400 MHz, DMSO- d₆) δ 9.02 (d, J = 2.1 Hz, 1H), 8.50 (d, J = 2.1 Hz, 1H), 8.23 (d, J = 8.4 Hz, 1H), 8.09 (d, J = 8.4 Hz, 1H), 7.48 (s, 1H), 6.51 (br s, 1H) 6.17 (td, J = 57.5, 3.7 Hz, 1H), 2.68- 2.59 (m, 3H), 2.56 (s, 3H), 2.45- 2.41 (m, 2H) | Alternate condition (5) was used |
| | cis-3-(difluoromethyl)-1-(6-(2- | | |
| | methyl-3H-imidazo[4,5- | | |
| | b]pyridin-6-yl)thieno[2,3- | | |
| | b]pyridin-2-yl)cyclobutanol | | |
| C95C | 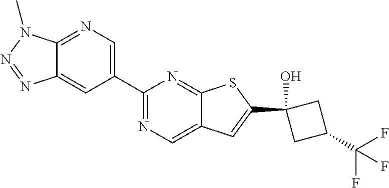 [SMILES: Cn1nnc2cc(-c3ncc4cc([C@]5(O)C[C@@H](C(F)(F)F)C5)sc4n3)cnc21] | 407.2; ¹H NMR (401 MHz, DMSO- d₆) δ 9.82 (d, J = 1.8 Hz, 1H), 9.42- 9.35 (m, 2H), 7.75 (s, 1H), 6.89 (s, 1H), 4.38 (s, 3H), 3.14 (dt, J = 17.9, 8.9 Hz, 1H), 2.83-2.73 (m, 2H), 2.60 (dd, J = 10.1, 2.9 Hz, 2H) | Alternate conditions (5) and (7) were used |
| | cis-1-(2-(3-methyl-3H- | | |
| | [1,2,3]triazolo[4,5-b]pyridin-6- | | |
| | yl)thieno[2,3-d]pyrimidin-6-yl)- | | |
| | 3-(trifluoromethyl)cyclobutanol | | |
| C96C | 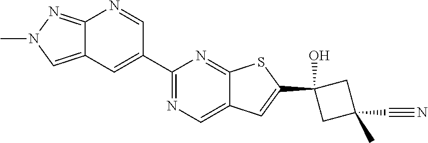 [SMILES: Cn1cc2cc(-c3ncc4cc([C@]5(O)C[C@@](C)(C#N)C5)sc4n3)cnc2n1] | 377.2; ¹H NMR (401 MHz, DMSO- d₆) δ 9.64 (d, J = 2.2 Hz, 1H), 9.32 (s, 1H), 9.23 (d, J = 2.2 Hz, 1H), 8.60 (s, 1H), 7.60 (s, 1H), 6.83 (s, 1H), 4.25 (s, 3H), 2.93-2.85 (m, 2H), 2.81-2.73 (m, 2H), 1.56 (s, 3H) | Alternate conditions (5) and (7) were used; After Step 1, the sample was purified by SFC Peak 2/SFC: Chiralpak AS-H 250 × 30 mm, 5 μm; Mobile phase: 15% (1:1) MeOH:ACN in CO₂ using a flow rate of 120 mL/min |
| | cis-3-hydroxy-1-methyl-3-(2-(2- | | |
| | methyl-2H-pyrazolo[3,4- | | |
| | b]pyridin-5-yl)thieno[2,3- | | |
| | d]pyrimidin-6- | | |
| | yl)cyclobutanecarbonitrile | | |
| C96T | 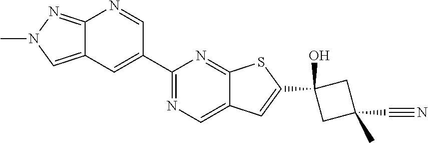 [SMILES: Cn1cc2cc(-c3ncc4cc([C@]5(O)C[C@](C)(C#N)C5)sc4n3)cnc2n1] | 377.2; ¹H NMR (401 MHz, DMSO- d₆) δ 9.64 (d, J = 2.2 Hz, 1H), 9.37 (s, 1H), 9.24 (d, J = 2.3 Hz, 1H), 8.60 (s, 1H), 7.66 (s, 1H), 6.78 (s, 1H), 4.25 (s, 3H), 3.21-3.13 (m, 2H), 2.64-2.56 (m, 2H), 1.68 (s, 3H) | Alternate conditions (5) and (7) were used; After Step 1, the sample was purified by SFC Peak 1/SFC: Chiralpak AS-H 250 × 30 mm, 5 μm; Mobile phase: 15% (1:1) MeOH:ACN in CO₂ using a flow rate of 120 mL/min |
| | trans-3-hydroxy-1-methyl-3-(2- | | |
| | (2-methyl-2H-pyrazolo[3,4- | | |
| | b]pyridin-5-yl)thieno[2,3- | | |
| | d]pyrimidin-6- | | |
| | yl)cyclobutanecarbonitrile | | |
| C97C | 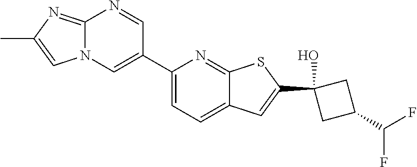 [SMILES: Cc1cn2cc(-c3ccc4cc([C@]5(O)C[C@@H](C(F)F)C5)sc4n3)cnc2n1] | 387.2; ¹H NMR (400 MHz, DMSO- d₆) δ 9.68 (d, J = 2.5 Hz, 1H), 9.22 (d, J = 2.5 Hz, 1H), 8.30 (d, J = 8.4 Hz, 1H), 8.10 (d, J = 8.4 Hz, 1H), 7.79 (s, 1H), 7.52 (s, 1H), 6.53 (s, 1H), 6.17 (td, J = 57.5, 3.5 Hz, 1H), 2.68-2.60 (m, 3H), 2.48-2.38 (m, 5H) ¹⁹F NMR (377 MHz, DMSO-d₆) δ- 121.09 (d, J = 12.6 Hz), -121.24 (d, J = 12.9 Hz) | Alternate condition (5) was used; |
| | cis-3-(difluoromethyl)-1-(6-(2- | | |
| | methylimidazo[1,2-a]pyrimidin- | | |
| | 6-yl)thieno[2,3-b]pyridin-2- | | |
| | yl)cyclobutanol | | |
| C98T | 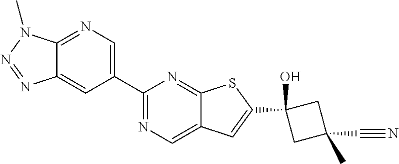 [SMILES: Cn1nnc2cc(-c3ncc4cc([C@]5(O)C[C@](C)(C#N)C5)sc4n3)cnc21] | 378.2; ¹H NMR (401 MHz, DMSO- d₆) δ 9.81 (d, J = 1.9 Hz, 1H), 9.43 (s, 1H), 9.39 (d, J = 1.9 Hz, 1H), 7.71 (s, 1H), 6.82 (s, 1H), 4.38 (s, 3H), 3.22-3.14 (m, 2H), 2.62 (d, J = 13.1 Hz, 2H), 1.69 (s, 3H) | Alternate conditions (5) and (7) were used; After Step 1, the sample was purified by SFC Peak 1/SFC: Chiralpak AS-H 250 × 30 mm, 5 μm; Mobile phase: 15% (1:1) MeOH:ACN in CO₂ using a flow rate of 120 mL/min |
| | trans-3-hydroxy-1-methyl-3-(2- | | |
| | (3-methyl-3H- | | |
| | [1,2,3]triazolo[4,5-b]pyridin-6- | | |
| | yl)thieno[2,3-d]pyrimidin-6- | | |
| | yl)cyclobutanecarbonitrile | | |
| C98C | 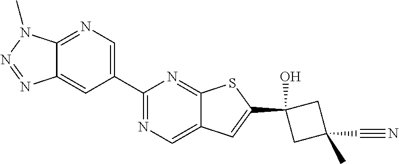 [SMILES: Cn1nnc2cc(-c3ncc4cc([C@]5(O)C[C@@](C)(C#N)C5)sc4n3)cnc21] | 378.2; ¹H NMR (401 MHz, DMSO- d₆) δ 9.81 (d, JJ = 1.9 Hz, 1H), 9.39 (br s, 2H), 7.65 (s, 1H), 6.88 (s, 1H), 4.38 (s, 3H), 2.90 (d, JJ = 13.2 Hz, 2H), 2.79 (d, JJ = 13.2 Hz, 2H), 1.57 (s, 3H) | Alternate conditions (5) and (7) were used; After Step 1, the sample was purified by SFC Peak 2/SFC: Chiralpak AS-H 250 × 30 mm, 5 μm; Mobile phase: 15% (1:1) MeOH:ACN in CO₂ using a flow rate of 120 mL/min |
| | cis-3-hydroxy-1-methyl-3-(2-(3- | | |
| | methyl-3H-[1,2,3]triazolo[4,5- | | |
| | b]pyridin-6-yl)thieno[2,3- | | |
| | d]pyrimidin-6- | | |
| | yl)cyclobutanecarbonitrile | | |
| C99C | 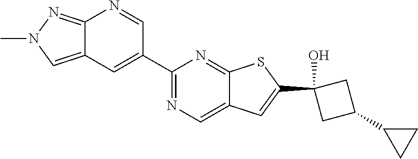 [SMILES: Cn1cc2cc(-c3ncc4cc([C@]5(O)C[C@@H](C6CC6)C5)sc4n3)cnc2n1] | 378.2; ¹H NMR (401 MHz, DMSO- d₆) δ 9.63 (d, J = 2.2 Hz, 1H), 9.27 (s, 1H), 9.22 (d, J = 2.3 Hz, 1H), 8.59 (s, 1H), 7.50 (s, 1H), 6.36 (s, 1H), 4.25 (s, 3H), 2.64-2.55 (m, 2H), 2.14 (td, J = 9.5, 2.9 Hz, 2H), 1.82 (p, J = 8.5 Hz, 1H), 0.90 (dq, J = 7.9, 4.1, 3.0 Hz, 1H), 0.46-0.35 (m, 2H), 0.15 (t, J = 5.0 Hz, 2H) | Alternate conditions (5) and (7) were was used |
| | cis-3-cyclopropyl-1-(2-(2- | | |
| | methyl-2H-pyrazolo[3,4- | | |
| | b]pyridin-5-yl)thieno[2,3- | | |
| | d]pyrimidin-6-yl)cyclobutanol | | |
| C102T | 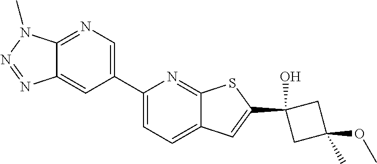 [SMILES: CO[C@]1(C)C[C@](O)(c2cc3ccc(-c4cnc5c(c4)nnn5C)nc3s2)C1] | 382.2; ¹H NMR (400 MHz, DMSO- d₆) δ 9.58 (d, J = 1.9 Hz, 1H), 9.21 (d, J = 2.0 Hz, 1H), 8.33 (d, J = 8.4 Hz, 1H), 8.23 (d, J = 8.4 Hz, 1H), 7.36 (s, 1H), 6.30 (s, 1H), 4.37 (s, 3H), 3.14 (s, 3H), 2.67-2.59 (m, 2H), 2.41-2.32 (m, 2H), 1.50 (s, 3H) | Alternate conditions (5) and (7) were used; First eluting isomer after step (1): purification by silica gel chromatography (eluent of 15-30% EtOAC in hexanes) |
| | trans-3-methoxy-3-methyl-1-(6- | | |
| | (3-methyl-3H- | | |
| | [1,2,3]triazolo[4,5-b]pyridin-6- | | |
| | yl)thieno[2,3-b]pyridin-2- | | |
| | yl)cyclobutanol | | |
| C102C | 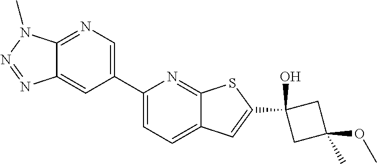 [SMILES: CO[C@]1(C)C[C@@](O)(c2cc3ccc(-c4cnc5c(c4)nnn5C)nc3s2)C1] | 382.2; ¹H NMR (400 MHz, DMSO- d₆) δ 9.57 (d, J = 2.0 Hz, 1H), 9.21 (d, J = 2.0 Hz, 1H), 8.34 (d, J = 8.4 Hz, 1H), 8.24 (d, J = 8.4 Hz, 1H), 7.44 (s, 1H), 6.26 (s, 1H), 4.37 (s, 3H), 3.15 (s, 3H), 2.69- 2.53 (m, 4H), 1.26 (s, 3H) | Alternate conditions (5) and (7) were used; Second eluting isomer after step (1): purification by silica gel chromatography (eluent of 15-30% EtOAc in hexanes) |
| | cis-3-methoxy-3-methyl-1-(6-(3- | | |
| | methyl-3H-[1,2,3]triazolo[4,5- | | |
| | b]pyridin-6-yl)thieno[2,3- | | |
| | b]pyridin-2-yl)cyclobutanol | | |
| C103C | 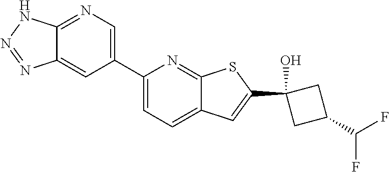 [SMILES: O[C@]1(c2cc3ccc(-c4cnc5[nH]nnc5c4)nc3s2)C[C@H](C(F)F)C1] | 374.2; ¹H NMR (400 MHz, DMSO- d₆) δ 9.46 (d, J = 2.0 Hz, 1H), 9.02 (d, J = 2.1 Hz, 1H), 8.31 (d, J = 8.4 Hz, 1H), 8.22 (d, J = 8.4 Hz, 1H), 7.53 (s, 1H), 6.53 (s, 1H), 6.18 (td, J = 57.4, 3.7 Hz, 1H), 2.70-2.56 (m, 3H), 2.45 (d, J = 2.8 Hz, 2H) ¹⁹F NMR (377 MHz, DMSO-d₆) δ- 121.15 | Alternate conditions (5) and (6) were used; the product was purified by SFC, using a Chiralpak IC 150 × 50 mm, 5 μm column; Mobile phase: 40% (1:1) MeOH:ACN in CO₂ with a flow rate of 150 mL/min |
| | cis-3-(difluoromethyl)-1-(6-(3H- | | |
| | [1,2,3]triazolo[4,5-b]pyridin-6- | | |
| | yl)thieno[2,3-b]pyridin-2- | | |
| | yl)cyclobutanol | | |
| C104T | 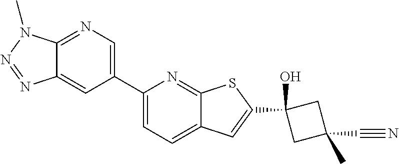 [SMILES: Cn1nnc2cc(-c3ccc4cc([C@]5(O)C[C@](C)(C#N)C5)sc4n3)cnc21] | 377.2; ¹H NMR (401 MHz, DMSO- d₆) δ 9.59 (d, J = 2.0 Hz, 1H), 9.23 (d, J = 1.9 Hz, 1H), 8.38 (d, J = 8.4 Hz, 1H), 8.26 (d, J = 8.4 Hz, 1H), 7.57 (s, 1H), 6.66 (s, 1H), 4.37 (s, 3H), 3.19-3.11 (m, 2H), 2.63- 2.56 (m, 2H), 1.68 (s, 3H) | Alternate condition (5) was used; After Step 1, the sample was purified by SFC Peak 1/SFC: Cellulose SZ 250 × 4.6 mm, 5 μm column; Mobile phase: 50% (1:1) MeOH:ACN in CO₂ and a flow rate of 120 mL/min |
| | trans-3-hydroxy-1-methyl-3-(6- | | |
| | (3-methyl-3H- | | |
| | [1,2,3]triazolo[4,5-b]pyridin-6- | | |
| | yl)thieno[2,3-b]pyridin-2- | | |
| | yl)cyclobutanecarbonitrile | | |
| C107 | 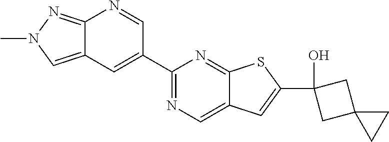 [SMILES: Cn1cc2cc(-c3ncc4cc(C5(O)CC6(CC6)C5)sc4n3)cnc2n1] | 364.2; ¹H NMR (400 MHz, DMSO- d₆) δ 9.64 (d, J = 2.2 Hz, 1H), 9.33 (s, 1H), 9.23 (d, J = 2.2 Hz, 1H), 8.59 (d, J = 0.6 Hz, 1H), 7.57 (s, 1H), 6.55 (s, 1H), 4.25 (s, 3H), 2.71- 2.77 (m, 2H), 2.47 (d, J = 2.7 Hz, 2H), 0.60-0.66 (m, 2H), 0.56 (ddd, J = 9.7, 6.0, 2.0 Hz, 2H) | Alternate conditions (5) and (7) were used |
| | 5-(2-(2-methyl-2H-pyrazolo[3,4- | | |
| | b]pyridin-5-yl)thieno[2,3- | | |
| | d]pyrimidin-6- | | |
| | yl)spiro[2.3]hexan-5-ol | | |
| C108 | 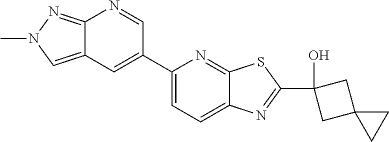 [SMILES: Cn1cc2cc(-c3ccc4nc(C5(O)CC6(CC6)C5)sc4n3)cnc2n1] | 364.2; ¹H NMR (400 MHz, DMSO- d₆) δ 9.40 (d, J = 2.4 Hz, 1H), 8.96 (d, J = 2.3 Hz, 1H), 8.56 (s, 1H), 8.49 (d, J = 8.6 Hz, 1H), 8.25 (d, J = 8.6 Hz, 1H), 6.95 (s, 1H), 4.25 (s, 3H), 2.89-2.79 (m, 2H), 2.61- 2.53 (m, 2H), 0.70-0.44 (m, 4H) | Alternate conditions (5) and (7) were used |
| | 5-(5-(2-methyl-2H-pyrazolo[3,4- | | |
| | b]pyridin-5-yl)[1,3]thiazolo[5,4- | | |
| | b]pyridin-2-yl)spiro [2.3]hexan- | | |
| | 5-ol | | |
| C109C | 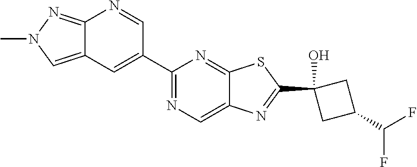 [SMILES: Cn1cc2cc(-c3ncc4nc([C@]5(O)C[C@@H](C(F)F)C5)sc4n3)cnc2n1] | 389.2; ¹H NMR (401 MHz, DMSO- d₆) δ 9.64 (d, J = 2.2 Hz, 1H), 9.54 (s, 1H), 9.25 (d, J = 2.2 Hz, 1H), 8.61 (s, 1H), 7.30 (br s, 1H), 6.20 (dt, J =57.2, 4.0 Hz, 1H), 4.26 (s, 3H), 2.93-2.81 (m, 1H), 2.79- 2.70 (m, 2H), 2.49-2.44 (m, 2H) | Alternate conditions (5) and (8) were used |
| | cis-3-(difluoromethyl)-1-(5-(2- | | |
| | methyl-2H-pyrazolo[3,4- | | |
| | b]pyridin-5-yl)[1,3]thiazolo[5,4- | | |
| | d\|pyrimidin-2-yl)cyclobutanol | | |
| C104C | 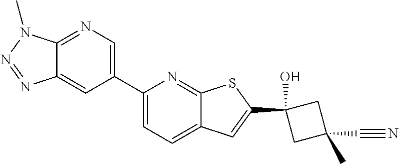 [SMILES: Cn1nnc2cc(-c3ccc4cc([C@]5(O)C[C@@](C)(C#N)C5)sc4n3)cnc21] | 377.2; ¹H NMR (401 MHz, DMSO- d₆) δ 9.58 (d, J = 2.0 Hz, 1H), 9.22 (d, J = 1.9 Hz, 1H), 8.34 (d, J = 8.4 Hz, 1H), 8.25 (d, J = 8.4 Hz, 1H), 7.49 (s, 1H), 6.71 (s, 1H), 4.37 (s, 3H), 2.94-2.85 (m, 2H), 2.81- 2.72 (m, 2H), 1.56 (s, 3H) | Alternate condition (5) was used; After Step 1, the sample was purified by SFC Peak 2/SFC: Cellulose SZ 250 × 4.6 mm, 5 μm column; Mobile phase: 50% (1:1) MeOH:ACN in CO₂ and a flow rate of 120 mL/min |
| | cis-3-hydroxy-1-methyl-3-(6-(3- | | |
| | methyl-3H-[1,2,3]triazolo[4,5- | | |
| | b]pyridin-6-yl)thieno[2,3- | | |
| | b]pyridin-2- | | |
| | yl)cyclobutanecarbonitrile | | |
| C113T | 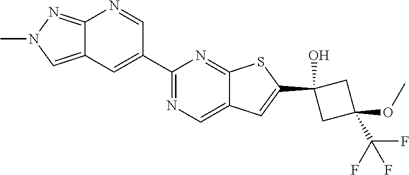 | 436.2; ¹H NMR (401 MHz, DMSO- d₆) δ 9.64 (d, J = 2.2 Hz, 1H), 9.36 (s, 1H), 9.24 (d, J = 2.2 Hz, 1H), 8.60 (s, 1H), 7.56 (s, 1H), 6.85 (s, 1H), 4.25 (s, 3H), 3.38 (s, 3H), 2.98- 2.89 (m, 2H), 2.81-2.73 (m, 2H) | Alternate conditions (5) and (7) were used; First eluting isomer after step (1): purification by silica gel chromatography (eluent of 5-20% EtOAC in hexanes) |
| | trans-3-methoxy-1-(2-(2-methyl- | | |
| | 2H-pyrazolo[3,4-b]pyridin-5- | | |
| | yl)thieno[2,3-d]pyrimidin-6-yl)- | | |
| | 3-(trifluoromethyl)cyclobutanol | | |
| C113C | 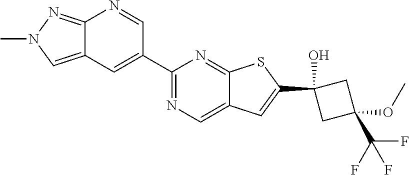 | 436.2; ¹H NMR (401 MHz, DMSO- d₆) δ 9.63 (d, J = 2.2 Hz, 1H), 9.33 (s, 1H), 9.23 (d, J = 2.2 Hz, 1H), 8.60 (s, 1H), 7.54 (s, 1H), 6.84 (s, 1H), 4.25 (s, 3H), 3.46 (s, 3H), 3.08- 3.00 (m, 2H), 2.82-2.72 (m, 2H) | Alternate conditions (5) and (7) were used; Second eluting isomer after step (1): purification by silica gel chromatography (eluent of 5-20% EtOAC in hexanes) |
| | cis-3-methoxy-1-(2-(2-methyl- | | |
| | 2H-pyrazolo[3,4-b]pyridin-5- | | |
| | yl)thieno[2,3-d]pyrimidin-6-yl)- | | |
| | 3-(trifluoromethyl)cyclobutanol | | |
| C114T | 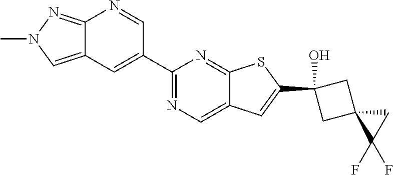 [SMILES: Cn1cc2cc(-c3ncc4cc([C@]5(O)C[C@@]6(CC6(F)F)C5)sc4n3)cnc2n1] | 400.0; ¹H NMR (400 MHz, DMSO- d₆) δ 9.64 (d, J = 2.2 Hz, 1H), 9.38 (s, 1H), 9.24 (d, J = 2.2 Hz, 1H), 8.60 (s, 1H), 7.46 (s, 1H), 6.88 (s, 1H), 4.25 (s, 3H), 2.79-2.63 (m, 4H), 1.57 (t, J = 8.4 Hz, 2H) | Alternate conditions (5) and (7) were used; After Step 1, the sample was purified by SFC Peak 1/SFC: Chiral Pak IB N-5 250 × 30 mm, 5 μm column; Mobile phase: 15% MeOH in CO₂ and a flow rate of 150 mL/min |
| | trans-1,1-difluoro-5-(2-(2- | | |
| | methyl-2H-pyrazolo[3,4- | | |
| | b]pyridin-5-yl)thieno[2,3- | | |
| | d]pyrimidin-6- | | |
| | yl)spiro[2.3]hexan-5-ol | | |
| C114C | 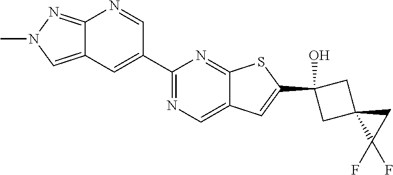 [SMILES: Cn1cc2cc(-c3ncc4cc([C@]5(O)C[C@]6(CC6(F)F)C5)sc4n3)cnc2n1] | 400.0; ¹H NMR (400 MHz, DMSO- d₆) δ 9.64 (d, J = 2.2 Hz, 1H), 9.33 (s, 1H), 9.24 (d, J = 2.2 Hz, 1H), 8.60 (s, 1H), 7.62 (s, 1H), 6.77 (s, 1H), 4.25 (s, 3H), 2.74 (d, J = 12.6 Hz, 2H), 2.66 (d, J = 12.6 Hz, 2H), 1.61 (t, J = 8.5 Hz, 2H) | Alternate conditions (5) and (7) were used; After Step 1, the sample was purified by SFC Peak 2/SFC: Chiral Pak IB N-5 250 × 30 mm, 5 μm column; Mobile phase: 15% MeOH in CO₂ and a flow rate of 150 mL/min |
| | cis-1,1-difluoro-5-(2-(2-methyl- | | |
| | 2H-pyrazolo[3,4-b]pyridin-5- | | |
| | yl)thieno[2,3-d]pyrimidin-6- | | |
| | yl)spiro[2.3]hexan-5-ol | | |
| C115T | 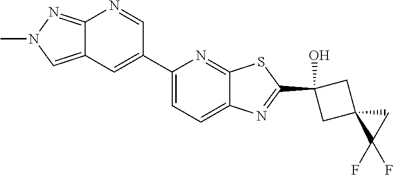 [SMILES: Cn1cc2cc(-c3ccc4nc([C@]5(O)C[C@@]6(CC6(F)F)C5)sc4n3)cnc2n1] | 400.0; ¹H NMR (400 MHz, DMSO- d₆) δ 9.40 (d, J = 2.3 Hz, 1H), 8.96 (d, J = 2.4 Hz, 1H), 8.57 (s, 1H), 8.48 (d, J = 8.6 Hz, 1H), 8.26 (d, J = 8.7 Hz, 1H), 7.28 (s, 1H), 4.25 (s, 3H), 2.90-3.00 (m, 2H), 2.64 (dt, J = 13.9, 3.1 Hz, 2H), 1.56 (t, J = 8.4 Hz, 2H) | Alternate conditions (5) and (7) were used; Second eluting isomer after step (1): purification by silica gel chromatography (eluent of 10-15% EtOAC in hexanes) |
| | trans-1,1-difluoro-5-(5-(2- | | |
| | methyl-2H-pyrazolo[3,4- | | |
| | b]pyridin-5-yl)[1,3]thiazolo[5,4- | | |
| | b]pyridin-2-yl)spiro[2.3]hexan- | | |
| | 5-ol | | |
| C115C | 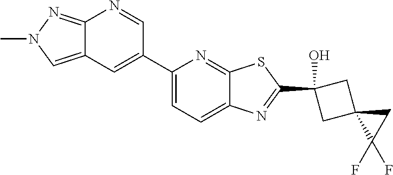 [SMILES: Cn1cc2cc(-c3ccc4nc([C@]5(O)C[C@]6(CC6(F)F)C5)sc4n3)cnc2n1] | 400.2; ¹H NMR (400 MHz, DMSO- d₆) δ 9.40 (d, J = 2.4 Hz, 1H), 8.97 (d, J = 2.3 Hz, 1H), 8.57 (s, 1H), 8.50 (d, J = 8.6 Hz, 1H), 8.27 (d, J = 8.7 Hz, 1H), 7.19 (br s, 1H), 4.25 (s, 3H), 2.91 (dt, J = 14.1, 2.9 Hz, 2H), 2.58-2.66 (m, 2H), 1.57 (t, J = 8.6 Hz, 2H) | Alternate conditions (5) and (7) were used; First eluting isomer after step (1): purification by silica gel chromatography (eluent of 10-15% EtOAC in hexanes) |
| | cis-1,1-difluoro-5-(5-(2-methyl- | | |
| | 2H-pyrazolo[3,4-b]pyridin-5- | | |
| | yl)[1,3]thiazolo[5,4-b]pyridin-2- | | |
| | yl)spiro[2.3]hexan-5-ol | | |
| C116 | 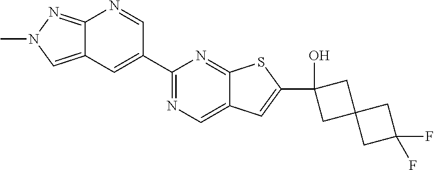 [SMILES: Cn1cc2cc(-c3ncc4cc(C5(O)CC6(CC(F)(F)C6)C5)sc4n3)cnc2n1] | 414.2; ¹H NMR (400 MHz, DMSO- d₆) δ 9.63 (d, J = 2.2 Hz, 1H), 9.29 (s, 1H), 9.22 (d, J = 2.2 Hz, 1H), 8.59 (s, 1H), 7.51 (s, 1H), 6.48 (s, 1H), 4.25 (s, 3H), 2.82-2.74 (m, 4H), 2.74-2.56 (m, 4H) | Alternate conditions (5) and (7) were used |
| | 6,6-difluoro-2-(2-(2-methyl-2H- | | |
| | pyrazolo[3,4-b]pyridin-5- | | |
| | yl)thieno[2,3-d]pyrimidin-6- | | |
| | yl)spiro[3.3]heptan-2-ol | | |
| C118T | 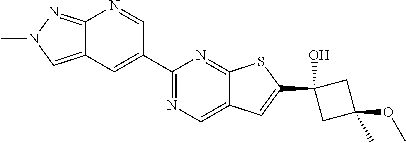 | 382.2; ¹H NMR (400 MHz, DMSO- d6)): 8 (ppm) 9.63 (d, J-2.2 Hz, 1H), 9.31 (s, 1H), 9.22 (d, J =2.2 Hz, 1H), 8.59 (s, 1H), 7.44 (s, 1H), 6.44 (s, 1H), 4.25 (s, 3H), 3.14 (s, 3H), 2.71-2.58 (m, 2H), 2.42- 2.32 (m, 2H), 1.49 (s, 3H) | Alternate conditions (5) and (7) were used After Step 1, the sample was purified by SFC Peak 1/SFC: LUX-C4 250 × 30 mm, 5 μm column; Mobile phase: 25% MeOH in CO₂ and a flow rate of 120 mL/min |
| | trans-3-methoxy-3-methyl-1-(2- | | |
| | (2-methyl-2H-pyrazolo[3,4- | | |
| | b]pyridin-5-yl)thieno[2,3- | | |
| | d\|pyrimidin-6-yl)cyclobutanol | | |
| C118C | 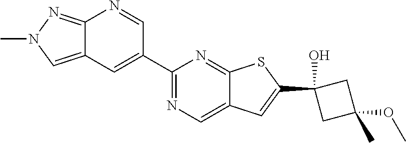 | 382.2; ¹H NMR (400 MHz, DMSO- d₆) δ (ppm) 9.63 (d, J =2.2 Hz, 1H), 9.30 (s, 1H), 9.23 (d, J =2.2 Hz, 1H), 8.59 (s, 1H), 7.53 (s, 1H), 6.39 (s, 1H), 4.25 (s, 3H),3.21 (s, 3H), 2.68-2.57 (m, 4H), 1.27 (s, 3H) | Alternate conditions (5) and (7) were used After Step 1, the sample was purified by SFC Peak 2/SFC: LUX-C4 250 × 30 mm, 5 μm column; Mobile phase: 25% MeOH in CO₂ and a flow rate of 120 mL/min |
| | cis-3-methoxy-3-methyl-1-(2-(2- | | |
| | methyl-2H-pyrazolo[3,4- | | |
| | b]pyridin-5-yl)thieno[2,3- | | |
| | d]pyrimidin-6-yl)cyclobutanol | | |
| C119C | 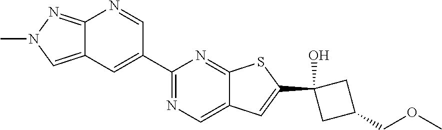 | 382.2; ¹H NMR (400 MHz, DMSO- d₆) δ 9.64 (d, J = 2.2 Hz, 1H), 9.29 (s, 1H), 9.23 (d, J = 2.2 Hz, 1H), 8.59 (s, 1H), 7.55 (s, 1H), 6.41 (s, 1H), 4.25 (s, 3H), 3.43 (d, J = 6.2 Hz, 2H), 3.28 (s, 3H), 2.58 (ddd, J = 10.1, 7.8, 2.6 Hz, 2H), 2.43-2.33 (m, 1H), 2.22 (td, J = 9.3, 2.7 Hz, 2H) | Alternate conditions (5) and (7) were used |
| | cis-3-(methoxymethyl)-1-(2-(2- | | |
| | methyl-2H-pyrazolo[3,4- | | |
| | b]pyridin-5-yl)thieno[2,3- | | |
| | d]pyrimidin-6-yl)cyclobutanol | | |
| C120C | 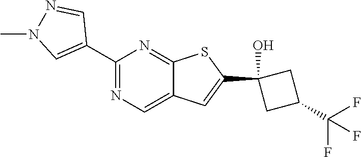 [SMILES: Cn1cc(-c2ncc3cc([C@]4(O)C[C@@H](C(F)(F)F)C4)sc3n2)cn1] | 355.2; ¹H NMR (400 MHz, DMSO- d₆) δ 9.12 (s, 1H), 8.45 (s, 1H), 8.09 (d, J = 0.7 Hz, 1H), 7.60 (s, 1H), 6.77 (s, 1H), 3.92 (s, 3H), 3.09 (h, J = 9.0 Hz, 1H), 2.79-2.70 (m, 2H), 2.63-2.52 (m, 2H) | Alternate conditions (5) and (7) were used |
| | cis-1-(2-(1-methyl-1H-pyrazol- | | |
| | 4-yl)thieno[2,3-d]pyrimidin-6- | | |
| | yl)-3- | | |
| | (trifluoromethyl)cyclobutanol | | |
| C121T | 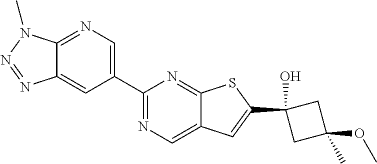 [SMILES: CO[C@]1(C)C[C@](O)(c2cc3cnc(-c4cnc5c(c4)nnn5C)nc3s2)C1] | 383.2; ¹H NMR (400 MHz, DMSO- d₆) δ 9.80 (d, J = 1.9 Hz, 1H), 9.40 -9.33 (m, 2H), 7.49 (s, 1H), 6.47 (s, 1H), 4.38 (s, 3H), 3.15 (s, 3H), 2.69-2.62 (m, 2H), 2.43-2.34 (m, 2H), 1.49 (s, 3H) | Alternate conditions (5) and (7) were used; First eluting isomer after step (1): purification by silica gel chromatography (eluent of 15-30% EtOAC in hexanes) |
| | trans-3-methoxy-3-methyl-1-(2- | | |
| | (3-methyl-3H- | | |
| | [1,2,3]triazolo[4,5-b]pyridin-6- | | |
| | yl)thieno[2,3-d]pyrimidin-6- | | |
| | yl)cyclobutanol | | |
| C121C | 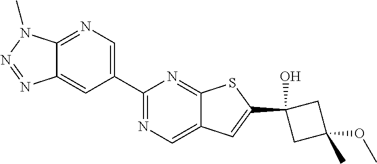 [SMILES: CO[C@]1(C)C[C@@](O)(c2cc3cnc(-c4cnc5c(c4)nnn5C)nc3s2)C1] | 383.2; ¹H NMR (400 MHz, DMSO- d₆) δ 9.81 (d, J = 1.9 Hz, 1H), 9.40- 9.35 (m, 2H), 7.58 (s, 1H), 6.44 (s, 1H), 4.38 (s, 3H), 3.15 (s, 3H), 2.69-2.53 (m, 4H), 1.28 (s, 3H) | Alternate conditions (5) and (7) were used; Second eluting isomer after step (1): purification by silica gel chromatography (eluent of 15-30% EtOAC in hexanes) |
| | cis-3-methoxy-3-methyl-1-(2-(3- | | |
| | methyl-3H-[1,2,3]triazolo[4,5- | | |
| | b]pyridin-6-yl)thieno[2,3- | | |
| | d\|pyrimidin-6-yl)cyclobutanol | | |
| C122C | 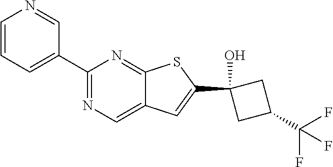 [SMILES: O[C@]1(c2cc3cnc(-c4cccnc4)nc3s2)C[C@H](C(F)(F)F)C1] | 352.2; ¹H NMR (401 MHz, DMSO- d₆) δ 9.59 (d, J = 2.2 Hz, 1H), 9.33 (s, 1H), 8.80-8.68 (m, 2H), 7.73 (s, 1H), 7.64-7.56 (m, 1H), 6.87 (s, 1H), 3.14 (h, J = 9.0 Hz, 1H), 2.82-2.71 (m, 2H), 2.59 (dd, J = 9.9, 3.0 Hz, 2H) | Alternate conditions (5) and (7) were used |
| | cis-1-(2-(3-pyridinyl)thieno[2,3- | | |
| | d]pyrimidin-6-yl)-3- | | |
| | (trifluoromethyl)cyclobutanol | | |
| C123C | 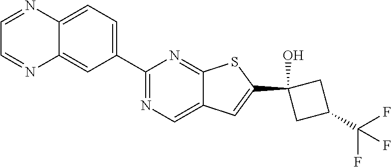 [SMILES: O[C@]1(c2cc3cnc(-c4ccc5nccnc5c4)nc3s2)C[C@H](C(F)(F)F)C1] | 403.2; ¹H NMR (401 MHz, DMSO- d₆) δ 9.40 (s, 1H), 9.14 (d, J = 1.9 Hz, 1H), 9.05 (d, J = 1.9 Hz, 1H), 9.02 (d, J = 1.8 Hz, 1H), 8.93 (dd, J = 8.8, 2.0 Hz, 1H), 8.27 (d, J = 8.9 Hz, 1H), 7.77 (s, 1H), 6.89 (s, 1H), 3.23-3.09 (m, 1H), 2.78 (ddd, J = 10.1, 6.5, 2.2 Hz, 2H), 2.59 (td, J = 9.8, 2.8 Hz, 2H) | Alternate conditions (5) and (7) were used |
| | cis-1-(2-(6- | | |
| | quinoxalinyl)thieno[2,3- | | |
| | d]pyrimidin-6-yl)-3- | | |
| | (trifluoromethyl)cyclobutanol | | |
| C124C | 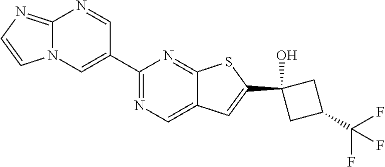 [SMILES: O[C@]1(c2cc3cnc(-c4cnc5nccn5c4)nc3s2)C[C@H](C(F)(F)F)C1] | 392.2; ¹H NMR (401 MHz, DMSO- d6) § 10.00 (d, J = 2.4 Hz, 1H), 9.47 (d, J = 2.4 Hz, 1H), 9.33 (s, 1H), 8.11 (d, J = 1.5 Hz, 1H), 7.81 (d, J = 1.4 Hz, 1H), 7.74 (s, 1H), 6.90 (s, 1H), 3.22-3.06 (m, 1H), 2.82- 2.73 (m, 2H), 2.58 (td, J = 9.8, 2.7 Hz, 2H) | Alternate conditions (5) and (7) were used |
| | cis-1-(2-(imidazo[1,2- | | |
| | a]pyrimidin-6-yl)thieno[2,3- | | |
| | d]pyrimidin-6-yl)-3- | | |
| | (trifluoromethyl)cyclobutanol | | |
| C125T | 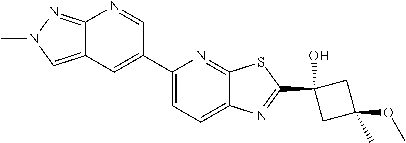 | 382.2; ¹H NMR (400 MHz, DMSO- d₆) δ 9.39 (d, J = 2.4 Hz, 1H), 8.96 (d, J = 2.4 Hz, 1H), 8.56 (s, 1H), 8.48 (d, J = 8.6 Hz, 1H), 8.25 (d, J = 8.6 Hz, 1H), 6.85 (s, 1H), 4.25 (s, 3H), 3.15 (s, 3H), 2.88-2.80 (m, 2H), 2.34-2.27 (m, 2H), 1.54 (s, 3H) | Alternate conditions (5) and (7) were used; After Step 1, the sample was purified by SFC Peak 1/SFC: ChiralPak IG 250 × 21 mm, 5 μm column; Mobile phase: 30% 1:1 MeOH/ACN in CO₂ and a flow rate of 100 mL/min |
| | trans-3-methoxy-3-methyl-1-(5- (2-methyl-2H-pyrazolo[3,4- b]pyridin-5-yl)[1,3]thiazolo[5,4- b]pyridin-2-yl)cyclobutanol | | |
| C125C | 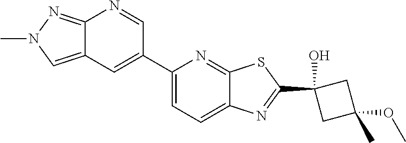 | 382.2; ¹H NMR (400 MHz, DMSO- d₆) δ 9.39 (d, J = 2.3 Hz, 1H), 8.96 (d, J = 2.3 Hz, 1H), 8.56 (s, 1H), 8.46 (d, J = 8.6 Hz, 1H), 8.24 (d, J = 8.6 Hz, 1H), 6.90 (s, 1H), 4.25 (s, 3H), 3.16 (s, 3H), 2.79-2.71 (m, 2H), 2.62-2.54 (m, 2H), 1.47 (s, 3H) | Alternate conditions (5) and (7) were used; After Step 1, the sample was purified by SFC Peak 2/SFC: ChiralPak IG 250 × 21 mm, 5 μm column; Mobile phase: 30% 1:1 MeOH/ACN in CO₂ and a flow rate of 100 mL/min |
| | cis-3-methoxy-3-methyl-1-(5-(2- | | |
| | methyl-2H-pyrazolo[3,4- | | |
| | b]pyridin-5-yl)[1,3]thiazolo[5,4- | | |
| | b]pyridin-2-yl)cyclobutanol | | |
| C126C | 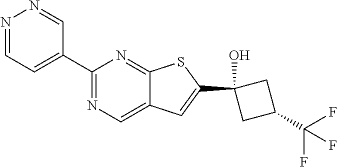 [SMILES: O[C@]1(c2cc3cnc(-c4ccnnc4)nc3s2)C[C@H](C(F)(F)F)C1] | 353.2; ¹H NMR (400 MHz, DMSO- d₆) δ 10.10 (dd, J = 2.4, 1.3 Hz, 1H), 9.46 (dd, J = 5.4, 1.3 Hz, 1H), 9.42 (s, 1H), 8.53 (dd, J = 5.4, 2.3 Hz, 1H), 7.80 (s, 1H), 6.94 (s, 1H), 3.22-3.11 (m, 1H), 2.82-2.72 (m, 2H), 2.59-2.56 (m, 2H) | Alternate conditions (5) and (7) were used |
| | cis-1-(2-(4- | | |
| | pyridazinyl)thieno[2,3- | | |
| | d]pyrimidin-6-yl)-3- | | |
| | (trifluoromethyl)cyclobutanol | | |
| C128C | 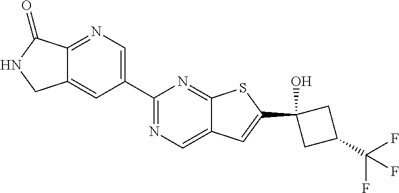 [SMILES: O=C1NCc2cc(-c3ncc4cc([C@]5(O)C[C@@H](C(F)(F)F)C5)sc4n3)cnc21] | 407.2; ¹H NMR (400 MHz, DMSO- d₆) δ 9.73 (d, J = 2.0 Hz, 1H), 9.37 (s, 1H), 9.10 (s, 1H), 8.99 (d, J = 1.9 Hz, 1H), 7.76 (s, 1H), 6.90 (s, 1H), 4.52 (s, 2H), 3.32-3.09 (m, 1H), 2.80-2.74 (m, 2H), 2.58- 2.50 (m, 2H) | Alternate conditions (5) and (7) were used |
| | 3-(6-(cis-1-hydroxy-3- | | |
| | (trifluoromethyl)cyclobutyl)thien | | |
| | o[2,3-d]pyrimidin-2-yl)-5,6- | | |
| | dihydro-7H-pyrrolo[3,4- | | |
| | b]pyridin-7-one | | |
| C129C | 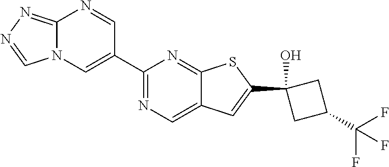 [SMILES: O[C@]1(c2cc3cnc(-c4cnc5nncn5c4)nc3s2)C[C@H](C(F)(F)F)C1] | 393.2; ¹H NMR (400 MHz, DMSO- d₆) δ 10.08 (d, J = 2.3 Hz, 1H), 9.81 (d, J = 2.3 Hz, 1H), 9.38 (s, 1H), 8.81 (s, 1H), 7.77 (s, 1H), 6.91 (br s, 1H), 3.19-3.12 (m, 1H), 2.80- 2.77 (m, 2H), 2.61-2.58 (m, 2H) | Alternate conditions (5) and (7) were used |
| | cis-1-(2-([1,2,4]triazolo[4,3- | | |
| | a]pyrimidin-6-yl)thieno[2,3- | | |
| | d]pyrimidin-6-yl)-3- | | |
| | (trifluoromethyl)cyclobutanol | | |
| C130T | 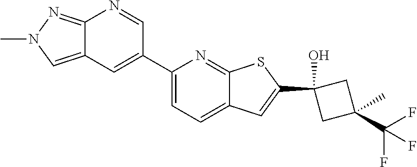 [SMILES: Cn1cc2cc(-c3ccc4cc([C@]5(O)C[C@](C)(C(F)(F)F)C5)sc4n3)cnc2n1] | 419.0; ¹H NMR (400 MHz, DMSO- d₆) δ 9.38 (d, J = 2.3 Hz, 1H), 8.92 (d, J = 2.3 Hz, 1H), 8.55 (s, 1H), 8.30 (d, J = 8.4 Hz, 1H), 8.11 (d, J = 8.4 Hz, 1H), 7.37 (s, 1H), 6.64 (s, 1H), 4.24 (s, 3H), 2.94-2.87 (m, 2H), 2.35-2.27 (m, 2H), 1.59 (s, 3H) | Alternate conditions (5) and (7) were used; After Step 1, the sample was purified by SFC Peak 1/SFC: ChiralPak IG 250 × 50 mm, 5 μm column; Mobile phase: 20% 1:1 MeOH/ACN in CO₂ and a flow rate of 180 mL/min |
| | trans-3-methyl-1-(6-(2-methyl- | | |
| | 2H-pyrazolo[3,4-b]pyridin-5- | | |
| | yl)thieno[2,3-b]pyridin-2-yl)-3- | | |
| | (trifluoromethyl)cyclobutanol | | |
| C130C | 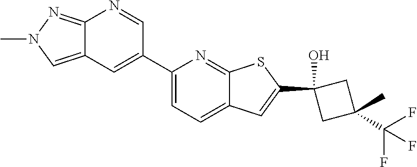 [SMILES: Cn1cc2cc(-c3ccc4cc([C@]5(O)C[C@@](C)(C(F)(F)F)C5)sc4n3)cnc2n1] | 419.0; ¹H NMR (400 MHz, DMSO- d₆) δ 9.39 (d, J = 2.3 Hz, 1H), 8.93 (d, J = 2.3 Hz, 1H), 8.55 (s, 1H), 8.30 (d, J = 8.4 Hz, 1H), 8.12 (d, J = 8.4 Hz, 1H), 7.55 (s, 1H), 6.47 (s, 1H), 4.25 (s, 3H), 2.78-2.70 (m, 2H), 2.61 (d, J = 13.7 Hz, 2H), 1.35 (s, 3H) | Alternate conditions (5) and (7) were used; After Step 1, the sample was purified by SFC Peak 2/SFC: ChiralPak IG 250 × 50 mm, 5 μm column; Mobile phase: 20% 1:1 MeOH/ACN in CO₂ and a flow rate of 180 mL/min |
| | cis-3-methyl-1-(6-(2-methyl-2H- | | |
| | pyrazolo[3,4-b]pyridin-5- | | |
| | yl)thieno[2,3-b]pyridin-2-yl)-3- | | |
| | (trifluoromethyl)cyclobutanol | | |
| C131C | 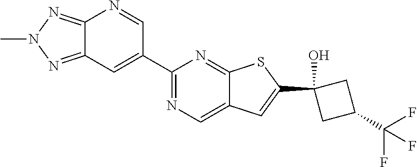 [SMILES: Cn1nc2cc(-c3ncc4cc([C@]5(O)C[C@@H](C(F)(F)F)C5)sc4n3)cnc2n1] | 407.0; ¹H NMR (400 MHz, DMSO- d₆) δ 9.84 (d, J = 2.0 Hz, 1H), 9.38 (s, 1H), 9.30 (d, J = 2.1 Hz, 1H), 7.76 (s, 1H), 6.89 (s, 1H), 4.62 (s, 3H), 3.18-3.12 (m, 1H), 2.80-2.75 (m, 2H), 2.68-2.61 (m, 2H) | Alternate conditions (5) and (7) were used |
| | cis-1-(2-(2-methyl-2H- | | |
| | [1,2,3]triazolo[4,5-b]pyridin-6- | | |
| | yl)thieno[2,3-d]pyrimidin-6-yl)- | | |
| | 3-(trifluoromethyl)cyclobutanol | | |
| C132C | 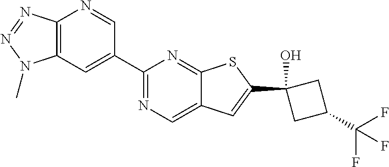 [SMILES: Cn1nnc2ncc(-c3ncc4cc([C@]5(O)C[C@@H](C(F)(F)F)C5)sc4n3)cc21] | 407.0; ¹H NMR (400 MHz, DMSO- d₆) δ 9.77 (d, J = 2.0 Hz, 1H), 9.39 (s, 1H), 9.33 (d, J = 2.0 Hz, 1H), 7.77 (s, 1H), 6.90 (s, 1H), 4.48 (s, 3H), 3.19-3.12 (m, 1H), 2.81-2.75 (m, 2H), 2.68-2.59 (m, 2H) | Alternate conditions (5) and (7) were used |
| | cis-1-(2-(1-methyl-1H- | | |
| | [1,2,3]triazolo[4,5-b]pyridin-6- | | |
| | yl)thieno[2,3-d]pyrimidin-6-yl)- | | |
| | 3-(trifluoromethyl)cyclobutanol | | |
| C133C | 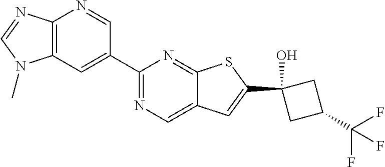 [SMILES: Cn1cnc2ncc(-c3ncc4cc([C@]5(O)C[C@@H](C(F)(F)F)C5)sc4n3)cc21] | 406.0; ¹H NMR (400 MHz, DMSO- d₆) δ 9.55 (d, J = 2.0 Hz, 1H), 9.33 (s, 1H), 8.98 (d, J = 2.0 Hz, 1H), 8.57 (s, 1H), 7.72 (s, 1H), 6.85 (br s, 1H), 4.00 (s, 3H), 3.20-3.08 (m, 1H), 2.82-2.73 (m, 2H), 2.63- 2.54 (m, 2H) | Alternate conditions (5) and (7) were used |
| | cis-1-(2-(1-methyl-1H- | | |
| | imidazo[4,5-b]pyridin-6- | | |
| | yl)thieno[2,3-d]pyrimidin-6-yl)- | | |
| | 3-(trifluoromethyl)cyclobutanol | | |
| C134C | 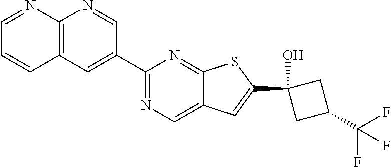 [SMILES: O[C@]1(c2cc3cnc(-c4cnc5ncccc5c4)nc3s2)C[C@H](C(F)(F)F)C1] | 403.6; ¹H NMR (400 MHz, DMSO- d₆) δ 10.08 (d, J = 2.5 Hz, 1H), 9.49 (d, J = 2.5 Hz, 1H), 9.41 (s, 1H), 9.17 (dd, J = 4.2, 2.0 Hz, 1H), 8.76- 8.74 (m, 1H), 7.78 (s, 1H), 7.75- 7.72 (m, 1H), 6.91 (br s, 1H), 3.19- 3.12 (m, 1H), 2.81-2.76 (m, 2H), 2.68-2.62 (m, 2H) | Alternate conditions (5) and (7) were used |
| | cis-1-(2-(1,8-naphthyridin-3- yl)thieno[2,3-d]pyrimidin-6-yl)- 3-(trifluoromethyl)cyclobutanol | | |
| C81T | 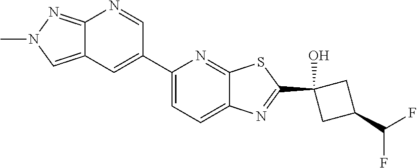 [SMILES: Cn1cc2cc(-c3ccc4nc([C@]5(O)C[C@H](C(F)F)C5)sc4n3)cnc2n1] | 388.2; ¹H NMR (400 MHz, DMSO- d₆) δ 9.39 (d, J = 2.4 Hz, 1H), 8.96 (d, J = 2.3 Hz, 1H), 8.56 (s, 1H), 8.48 (d, J = 8.6 Hz, 1H), 8.26 (d, J = 8.7 Hz, 1H), 7.09 (s, 1H), 6.19 (td, J = 57.2, 4.3 Hz, 1H), 4.25 (s, 3H), 2.94-2.79 (m, 1H), 2.76- 2.66 (m, 2H), 2.48-2.39 (m, 2H) | Alternate condition (5) was used; Minor eluting isomer from column chromatography after Step 1 |
| | trans-3-(difluoromethyl)-1-(5- | | |
| | (2-methyl-2H-pyrazolo[3,4- | | |
| | b]pyridin-5-yl)thiazolo[5,4- | | |
| | b]pyridin-2-yl)cyclobutanol | | |
| C94T | 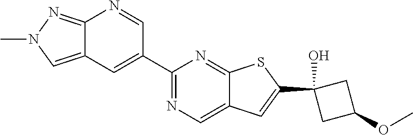 | 368.2; ¹H NMR (401 MHz, DMSO- d₆) δ 9.63 (d, J = 2.2 Hz, 1H), 9.30 (s, 1H), 9.22 (d, J = 2.2 Hz, 1H), 8.59 (s, 1H), 7.46 (s, 1H), 6.46 (s, 1H), 4.25 (s, 3H), 4.22-4.15 (m, 1H), 3.22 (s, 3H), 2.74-2.64 (m, 2H), 2.46-2.36 (m, 2H) | Alternate conditions (5) and (7) were used; After Step 1, the sample was purified by SFC Minor Isomer/SFC: ChiralPak IG 250 × 50 mm, 5 μm column; Mobile phase: 40% (30% DCM in 1:1 MeOH/ACN) in CO₂ and a flow rate of 180 mL/min |
| | trans-3-methoxy-1-(2-(2-methyl- | | |
| | 2H-pyrazolo[3,4-b]pyridin-5- | | |
| | yl)thieno[2,3-d]pyrimidin-6- | | |
| | yl)cyclobutanol | | |
| C137C | 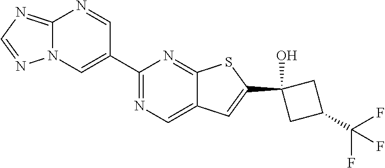 [SMILES: O[C@]1(c2cc3cnc(-c4cnc5ncnn5c4)nc3s2)C[C@H](C(F)(F)F)C1] | 393.0; ¹H NMR (400 MHz, DMSO- d₆) δ 10.08 (d, J = 2.4 Hz, 1H), 9.81 (d, J = 2.4 Hz, 1H), 9.38 (s, 1H), 8.81 (s, 1H), 7.77 (s, 1H), 6.91 (s, 1H), 3.19-3.12 (m, 1H), 2.83- 2.72 (m, 2H), 2.68-2.59 (m, 2H) | Alternate conditions (5) and (7) were used |
| | cis-1-(2-([1,2,4]triazolo[1,5- | | |
| | alpyrimidin-6-yl)thieno[2,3- | | |
| | d]pyrimidin-6-yl)-3- | | |
| | (trifluoromethyl)cyclobutan-1-ol | | |
| D2 | 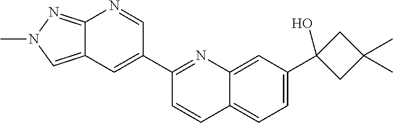 | 359.2; ¹H NMR (401 MHz, DMSO- d₆) δ 9.56 (d, J = 2.3 Hz, 1H), 9.04 (d, J = 2.3 Hz, 1H), 8.59 (s, 1H), 8.45 (d, J = 8.6 Hz, 1H), 8.22 (d, J = 8.6 Hz, 1H), 8.08 (d, J = 1.8 Hz, 1H), 7.99 (d, J = 8.5 Hz, 1H), 7.68 (dd, J = 8.5, 1.8 Hz, 1H), 5.57 (s, 1H), 4.26 (s, 3H), 2.50-2.43 (m, 2H), 2.26-2.19 (m, 2H), 1.38 (s, 3H), 1.12 (s, 3H) | Alternate condition (5) was used |
| | 3,3-dimethyl-1-(2-(2-methyl-2H- | | |
| | pyrazolo[3,4-b]pyridin-5-yl)-7- | | |
| | quinolinyl)cyclobutanol | | |
| D3R | 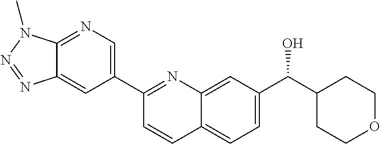 [SMILES: Cn1nnc2cc(-c3ccc4ccc([C@H](O)C5CCOCC5)cc4n3)cnc21] | 376.2; ¹H NMR (401 MHz, DMSO- d₆) δ 9.74 (d, J = 1.9 Hz, 1H), 9.35 (d, J = 1.9 Hz, 1H), 8.53 (d, J = 8.6 Hz, 1H), 8.36 (d, J = 8.6 Hz, 1H), 8.05-7.97 (m, 2H), 7.63 (dd, J = 8.4, 1.6 Hz, 1H), 5.48 (d, J = 4.5 Hz, 1H), 4.56 (dd, J = 6.7, 4.6 Hz, 1H), 4.39 (s, 3H), 3.92- 3.84 (m, 1H), 3.80 (dd, J = 11.2, 4.0 Hz, 1H), 3.26-3.14 (m, 2H), 1.92-1.80 (m, 1H), 1.71 (d, J = 13.2 Hz, 1H), 1.38-1.31 (m, 2H), 1.23-1.16 (m, 1H). | Alternate condition (5) was used; After Step 1, the sample was purified via SFC; Peak 1/SFC: Chiralcel OJ-H (250 × 4.6) mm, 5.0 um column; Mobile phase: 20% MeOH in CO₂ with a flow rate of 150 mL/min |
| | (R)-(2-(3-methyl-3H- | | |
| | [1,2,3]triazolo[4,5-b]pyridin-6- | | |
| | yl)-7-quinolinyl)(tetrahydro-2H- | | |
| | pyran-4-yl)methanol | | |
| D3S | 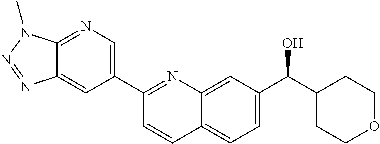 [SMILES: Cn1nnc2cc(-c3ccc4ccc([C@@H](O)C5CCOCC5)cc4n3)cnc21] | 376.2; ¹H NMR (401 MHz, DMSO- d₆) δ 9.74 (d, J = 2.0 Hz, 1H), 9.35 (d, J = 2.0 Hz, 1H), 8.53 (d, J = 8.6 Hz, 1H), 8.36 (d, J = 8.6 Hz, 1H), 8.05-7.97 (m, 2H), 7.63 (dd, J = 8.4, 1.6 Hz, 1H), 5.48 (d, J = 4.6 Hz, 1H), 4.56 (dd, J = 6.7, 4.6 Hz, 1H), 4.39 (s, 3H), 3.92-3.84 (m, 1H), 3.80 (dd, J = 11.5, 4.1 Hz, 1H), 3.22 (dtd, J = 18.2, 11.7, 2.1 Hz, 2H), 1.92- 1.80 (m, 1H), 1.71 (d, J = 13.2 Hz, 1H), 1.38-1.31 (m, 2H), 1.23- 1.16 (m, 1H). | Alternate condition (5) was used; After Step 1, the sample was purified via SFC; Peak 2/SFC: Chiralcel OJ-H (250 × 4.6) mm, 5.0 um column; Mobile phase: 20% MeOH in CO₂ with a flow rate of 150 mL/min |
| | (S)-(2-(3-methyl-3H- | | |
| | [1,2,3]triazolo[4,5-b]pyridin-6- | | |
| | yl)-7-quinolinyl)(tetrahydro-2H- | | |
| | pyran-4-yl)methanol | | |
| D4S | 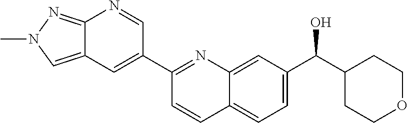 [SMILES: Cn1cc2cc(-c3ccc4ccc([C@@H](O)C5CCOCC5)cc4n3)cnc2n1] | 375.2; ¹H NMR (401 MHz, DMSO- d₆) δ 9.55 (d, J = 2.3 Hz, 1H), 9.03 (d, J = 2.3 Hz, 1H), 8.59 (s, 1H), 8.45 (d, J = 8.6 Hz, 1H), 8.22 (d, J = 8.6 Hz, 1H), 8.00-7.93 (m, 2H), 7.58 (dd, J = 8.4, 1.6 Hz, 1H), 5.45 (d, J = 4.5 Hz, 1H), 4.54 (dd, J = 6.8, 3.9 Hz, 1H), 4.26 (s, 3H), 3.88 (dd, J = 11.4, 4.1 Hz, 1H), 3.83- 3.75 (m, 1H), 3.22 (dtd, J = 19.4, 11.7, 2.2 Hz, 2H), 1.91-1.79 (m, 1H), 1.76-1.68 (m, 1H), 1.37 (qd, J = 12.2, 4.5 Hz, 2H), 1.18 (d, J = 12.3 Hz, 1H) | Alternate condition (5) was used; After Step 1, the sample was purified via SFC; Peak 1/SFC: Chiralcel OJ-H (250 × 4.6) mm, 5.0 um column; Mobile phase: 20% MeOH in CO₂ with a flow rate of 150 mL/min |
| | (S)-(2-(2-methyl-2H- | | |
| | pyrazolo[3,4-b]pyridin-5-yl)-7- | | |
| | quinolinyl)(tetrahydro-2H- | | |
| | pyran-4-yl)methanol | | |
| D4R | 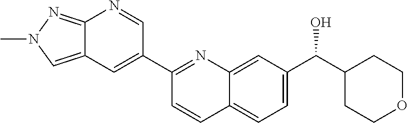 [SMILES: Cn1cc2cc(-c3ccc4ccc([C@H](O)C5CCOCC5)cc4n3)cnc2n1] | 375.2; ¹H NMR (401 MHz, DMSO- d₆) δ 9.55 (d, J = 2.3 Hz, 1H), 9.03 (d, J = 2.3 Hz, 1H), 8.59 (s, 1H), 8.45 (d, J = 8.6 Hz, 1H), 8.22 (d, J = 8.7 Hz, 1H), 8.00-7.93 (m, 2H), 7.58 (dd, J = 8.4, 1.7 Hz, 1H), 5.45 (d, J = 4.5 Hz, 1H), 4.54 (dd, J = 6.7, 4.6 Hz, 1H), 4.26 (s, 3H), 3.91- 3.83 (m, 1H), 3.83-3.76 (m, 1H), 3.27-3.14 (m, 2H), 1.88- 1.80 (m, 1H), 1.72 (d, J = 13.2 Hz, 1H), 1.37 (qd, J = 12.2, 4.5 Hz, 2H), 1.20 (t, J = 15.5 Hz, 1H) | Alternate condition (5) was used; After Step 1, the sample was purified via SFC; Peak 2/SFC: Chiralcel OJ-H (250 × 4.6) mm, 5.0 um column; Mobile phase: 20% MeOH in CO₂ with a flow rate of 150 mL/min |
| | (R)-(2-(2-methyl-2H- | | |
| | pyrazolo[3,4-b]pyridin-5-yl)-7- | | |
| | quinolinyl)(tetrahydro-2H- | | |
| | pyran-4-yl)methanol | | |
| D5C | 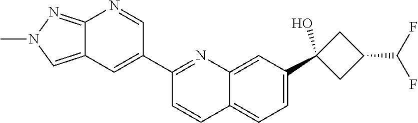 [SMILES: Cn1cc2cc(-c3ccc4ccc([C@]5(O)C[C@@H](C(F)F)C5)cc4n3)cnc2n1] | 381.2; ¹H NMR (401 MHz, DMSO- d₆) δ 9.57 (d, J = 2.3 Hz, 1H), 9.05 (d, J = 2.3 Hz, 1H), 8.59 (s, 1H), 8.48 (d, J = 8.5 Hz, 1H), 8.24 (d, J = 8.5 Hz, 1H), 8.17 (s, 1H), 8.02 (d, J = 8.3 Hz, 1H), 7.85-7.75 (m, 1H), 6.22 (td, J = 57.4, 4.8 Hz, 1H), 6.01 (s, 1H), 4.26 (s, 3H), 2.71- 2.52 (m, 3H), 2.43-2.38 (m, 2H) | Alternate condition (5) was used |
| | cis-3-(difluoromethyl)-1-(2-(2- | | |
| | methyl-2H-pyrazolo[3,4- | | |
| | b]pyridin-5-yl)-7- | | |
| | quinolinyl)cyclobutanol | | |
| D6C | 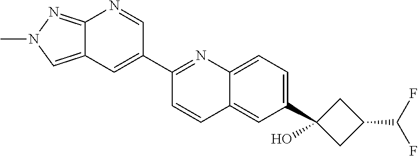 [SMILES: Cn1cc2cc(-c3ccc4cc([C@]5(O)C[C@@H](C(F)F)C5)ccc4n3)cnc2n1] | 381.0; ¹H NMR (401 MHz, DMSO- d₆) δ 9.56 (d, J = 2.3 Hz, 1H), 9.04 (d, J = 2.3 Hz, 1H), 8.59 (s, 1H), 8.52 (d, J = 8.7 Hz, 1H), 8.26 (d, J = 8.6 Hz, 1H), 8.16-8.07 (m, 2H), 7.96 (dd, J = 8.8, 2.1 Hz, 1H), 6.21 (dt, J = 57.6, 4.8 Hz, 1H), 5.99 (s, 1H), 4.26 (s, 3H), 2.72-2.62 (m, 2H), 2.58-2.54 (m, 1H), 2.44- 2.38 (m, 2H) | Alternate condition (5) was used |
| | cis-3-(difluoromethyl)-1-(2-(2- | | |
| | methyl-2H-pyrazolo[3,4- | | |
| | b]pyridin-5-yl)-6- | | |
| | quinolinyl)cyclobutanol | | |
| D7R | 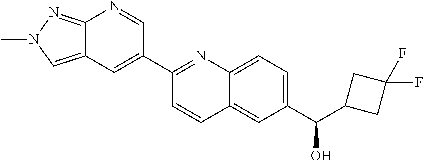 [SMILES: Cn1cc2cc(-c3ccc4cc([C@H](O)C5CC(F)(F)C5)ccc4n3)cnc2n1] | 381.2; ¹H NMR (401 MHz, DMSO- d₆) δ 9.54 (d, J = 2.3 Hz, 1H), 9.03 (d, J = 2.3 Hz, 1H), 8.59 (s, 1H), 8.48 (d, J = 8.6 Hz, 1H), 8.25 (d, J = 8.7 Hz, 1H), 8.06 (d, J = 8.7 Hz, 1H), 7.96 (d, J = 1.9 Hz, 1H), 7.80 (dd, J = 8.8, 2.0 Hz, 1H), 5.81 (d, J = 4.6 Hz, 1H), 4.77 (t, J = 4.8 Hz, 1H), 4.26 (s, 3H), 2.71-2.52 (m, 3H), 2.51-2.41 (m, 2H) | Alternate condition (5) was used; After Step 1, the sample was purified by SFC; Peak 2/SFC: Chiralpak AS-H 250 × 30 mm, 5um; Mobile phase: 20% IPA in CO₂ using a flow rate of 120 mL/min |
| | (R)-(3,3-difluorocyclobutyl)(2- | | |
| | (2-methyl-2H-pyrazolo[3,4- | | |
| | b]pyridin-5-yl)-6- | | |
| | quinolinyl)methanol | | |
| D7S | 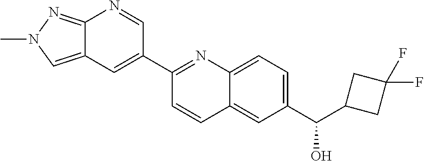 [SMILES: Cn1cc2cc(-c3ccc4cc([C@@H](O)C5CC(F)(F)C5)ccc4n3)cnc2n1] | 381.2; ¹H NMR (401 MHz, DMSO- d₆) δ 9.54 (d, J = 2.3 Hz, 1H), 9.03 (d, J = 2.3 Hz, 1H), 8.59 (s, 1H), 8.54-8.44 (m, 1H), 8.25 (d, J = 8.6 Hz, 1 H), 8.06 (d, J = 8.7 Hz, 1 H), 7.96 (d, J = 1.9 Hz, 1H), 7.80 (dd, J = 8.7, 2.0 Hz, 1H), 5.81 (d, J = 4.6 Hz, 1H), 4.77 (t, J = 4.8 Hz, 1H), 4.26 (s, 3H), 2.71- 2.52 (m, 3H), 2.44-2.37 (m, 2H) | Alternate condition (5) was used; After Step 1, the sample was purified by SFC; Peak 1/SFC: Chiralpak AS-H 250 × 30 mm, 5 μm; Mobile phase: 20% IPA in CO₂ using a flow rate of 120 mL/min |
| | (S)-(3,3-difluorocyclobutyl)(2- | | |
| | (2-methyl-2H-pyrazolo[3,4- | | |
| | b]pyridin-5-yl)-6- | | |
| | quinolinyl)methanol | | |

### Group 2

Example 2-1:6-(2-((R)-(3,3-difluorocyclobutyl)(hydroxy)methyl)thieno[2,3-b]pyridin-6-yl)-3-methyl-4(3H)-pyrimidinone B49R; 6-(2-((S)-(3,3-difluorocyclobutyl)(hydroxy)methyl)thieno[2,3-b]pyridin-6-yl)-3-methyl-4(3H)-pyrimidinone B49S

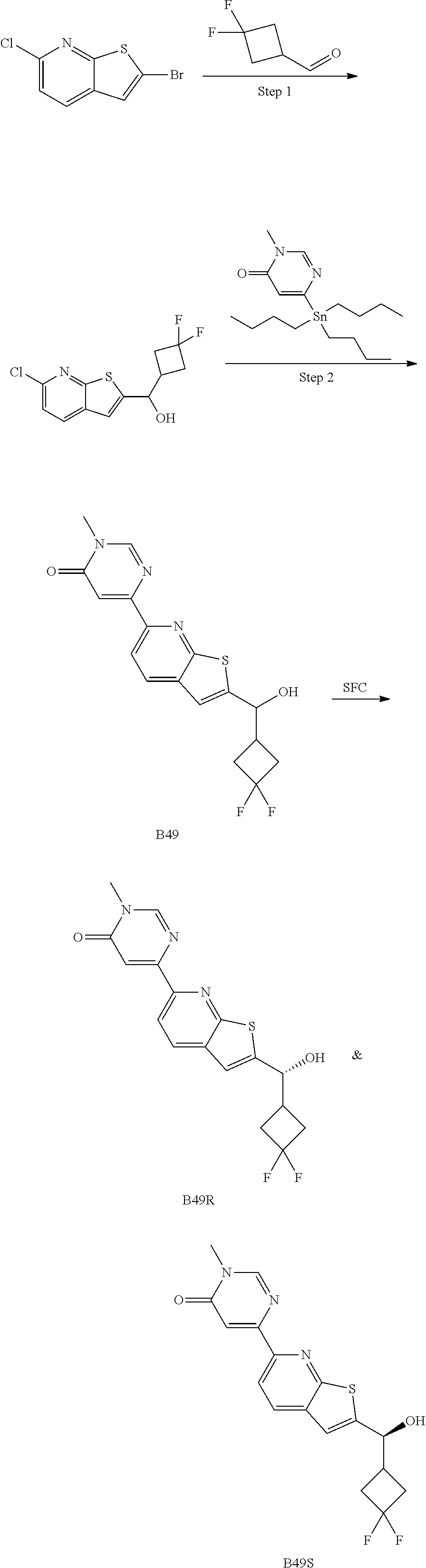

Step 1: (6-chlorothieno[2,3-b]pyridine-2-yl)(3,3-difluorocyclobutyl)methanol. To a solution of 2-bromo-6-chlorothieno[2,3-b]pyridine (5.00 g, 20.1 mmol) in THF (50 mL) was added a solution of n-butyllithium in hexanes (2.5 M, 8.45 mL, 21.1 mmol) at −78° C. The reaction mixture was stirred for 2 min, and 3,3-difluorocyclobutane-1-carbaldehyde (2.66 g, 22.1 mmol) in THF (50 mL) was added. The reaction mixture was stirred for additional 0.5 h at −78° C. The reaction mixture was diluted with H₂O (100 mL). Then the mixture was extracted with EtOAc (3×50 mL). The combined organic layers were washed with brine (50 mL), dried over Na₂SO₄, filtered and concentrated under reduced pressure. The residue was purified by chromatography, eluting with a gradient of 0-7% MeOH/EtOAc, to provide (6-chlorothieno[2,3-b]pyridin-2-yl)(3,3-difluorocyclobutyl)methanol (6.00 g, 20.7 mmol, 103% crude yield). ¹H NMR (CHLOROFORM-d) δ: 7.92 (d, J 8.4 Hz, 1H), 7.31 (d, J 8.3 Hz, 1H), 7.14 (s, 1H), 4.99 (br d, J 6.0 Hz, 1H), 2.66-2.74 (m, 2H), 2.57-2.62 (m, 2H), 2.44-2.54 (m, 1H).

Step 2: 6-(2-((3,3-difluorocyclobutyl)(hydroxy)methyl)thieno[2,3-b]pyridine-6-yl)-3-methylpyrimidin-4(3H)-one. Ten batches of this reaction were conducted in parallel. To a mixture of (6-chlorothieno[2,3-b]pyridine-2-yl)(3,3-difluorocyclobutyl)methanol (500 mg, 1.73 mmol) and 3-methyl-6-(tributylstannyl)pyrimidin-4(3H)-one (827 mg, 2.07 mmol) in 1,4-dioxane (5 mL) was added Pd(amphos)Cl₂ (122 mg, 0.17 mmol). The mixture was stirred at 100° C. for 12 h. The ten reaction mixtures were combined, diluted with H₂O (50 mL) and extracted with EtOAc (4×50 mL). The combined organic layers were washed with brine (3×30 mL), dried over Na₂SO₄, filtered and concentrated under reduced pressure to give a residue. The residue was purified by column chromatography, eluting with a gradient of 1%-33% EtOAc in pet. ether, to give Compound B49 (6.7 g, 18.44 mmol). m/z (ESI): 364.3 (M+H)⁺; ¹H NMR (400 MHz, DMSO-d₆) δ 8.62 (s, 1H), 8.29 (s, 2H), 7.38 (s, 1H), 7.27 (s, 1H), 6.31 (d, J 5.2 Hz, 1H), 4.94 (br t, J 4.4 Hz, 1H), 3.47 (s, 3H), 2.57-2.54 (m, 5H).

Step 3: SFC Purification. The sample was purified via SFC using a Chiralpak IC-3, 50×4.6 mm, 3 μm column with a mobile phase of 50% MeOH with 0.1% IPAm using a flowrate of 3.4 mL/min to generate peak 1 and peak 2:

Peak 1: B49S. m/z (ESI): 364.0 (M+H)⁺. ¹H NMR (400 MHz, DMSO-d₆) δ 8.62 (s, 1H), 8.33-8.27 (m, 2H), 7.38 (s, 1H), 7.27 (s, 1H), 6.31 (d, J 4.8 Hz, 1H), 4.94 (br s, 1H), 3.47 (s, 3H), 2.54-2.60 (m, 5H).

Peak 2: B49R. m/z (ESI): 364.0 (M+H)⁺. ¹H NMR (400 MHz, DMSO-d₆) δ: 8.62 (s, 1H), 8.29 (s, 2H), 7.38 (s, 1H), 7.27 (s, 1H), 6.32 (br d, J 2.4 Hz, 1H), 4.94 (br s, 1H), 3.47 (s, 3H), 2.53-2.67 (m, 5H).

### Group 3

Example 3-1:3,3-difluoro-1-(6-(2-methyl-2H-pyrazolo[3,4-b]pyridin-5-yl)thieno[2,3-b]pyridin-2-yl)cyclobutanol C₅

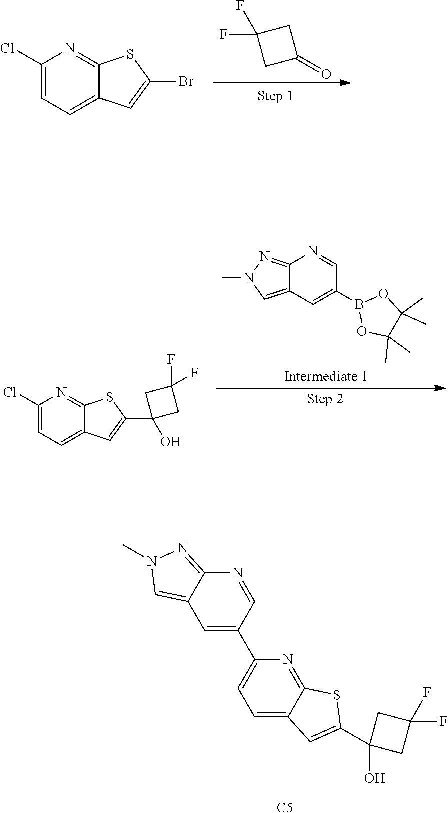

Step 1: 1-(6-chlorothieno[2,3-b]pyridin-2-yl)-3,3-difluorocyclobutan-1-ol. To a solution of 2-bromo-6-chlorothieno[2,3-b]pyridine (0.5 g, 2.01 mmol) in THF (2 mL) was added n-butyllithium in hexanes (2.5 M, 1.21 mL, 3.02 mmol) at −78° C. The reaction mixture was stirred for 5 min, and 3,3-difluorocyclobutan-1-one (0.24 g, 2.21 mmol) in THF (0.5 mL) was added. The reaction mixture was quenched by addition of sat. aq. NH₄Cl (10 mL). Then the mixture was extracted with EtOAc (10 mL×3), and the combined organic layers were washed with brine (50 mL), dried over Na₂SO₄, filtered and the concentrated under reduced pressure. The residue was purified by preperative thin-layer chromatography, eluting with 1:1 pet. ether/EtOAc, to afford 1-(6-Chlorothieno[2,3-b]pyridin-2-yl)-3,3-difluorocyclobutan-1-ol (0.028 g, 0.10 mmol, 5% yield). m/z (ESI): 276.0 (M+H)⁺.

Step 2: 3,3-difluoro-1-(6-(2-methyl-2H-pyrazolo[3,4-b]pyridin-5-yl)thieno[2,3-b]pyridin-2-yl)cyclobutanol. 1-(6-Chlorothieno[2,3-b]pyridin-2-yl)-3,3-difluorocyclobutan-1-ol (28.0 mg, 0.102 mmol), Intermediate 1 (31.6 mg, 0.12 mmol), K₂CO₃ (28.1 mg, 0.20 mmol) and Pd(dppf)Cl₂ (7.43 mg, 0.010 mmol) were taken up into a microwave tube in 1,4-dioxane (1.5 mL) and H₂O (0.5 mL). The sealed tube was heated at 120° C. for 1 h under microwave irradiation. The reaction mixture was directly purified by prep-HPLC, eluting with 25-55% ACN in H₂O (0.2% NH₄HCO₃), to give Compound C₅ (4.0 mg, 0.010 mmol, 10% yield). m/z (ESI): 373.1 (M+H)⁺. ¹H NMR (400 MHz, DMSO-d₆) δ 9.38 (d, J 2.3 Hz, 1H) 8.92 (d, J 2.3 Hz, 1H) 8.54 (s, 1H) 8.29 (d, J 8.4 Hz, 1H) 8.12 (d, J 8.4 Hz, 1H) 7.46 (s, 1H) 6.84 (s, 1H) 4.24 (s, 3H) 3.19-3.28 (m, 2H) 2.99-3.11 (m, 2H).

### Group 4

Example 4-1: (R)-cyclopentyl(6-(2-methyl-2H-pyrazolo[3,4-b]pyridin-5-yl)-1-benzothiophen-2-yl)methanol B58R; (S)-cyclopentyl(6-(2-methyl-2H-pyrazolo[3,4-b]pyridin-5-yl)-1-benzothiophen-2-yl)methanol B58S

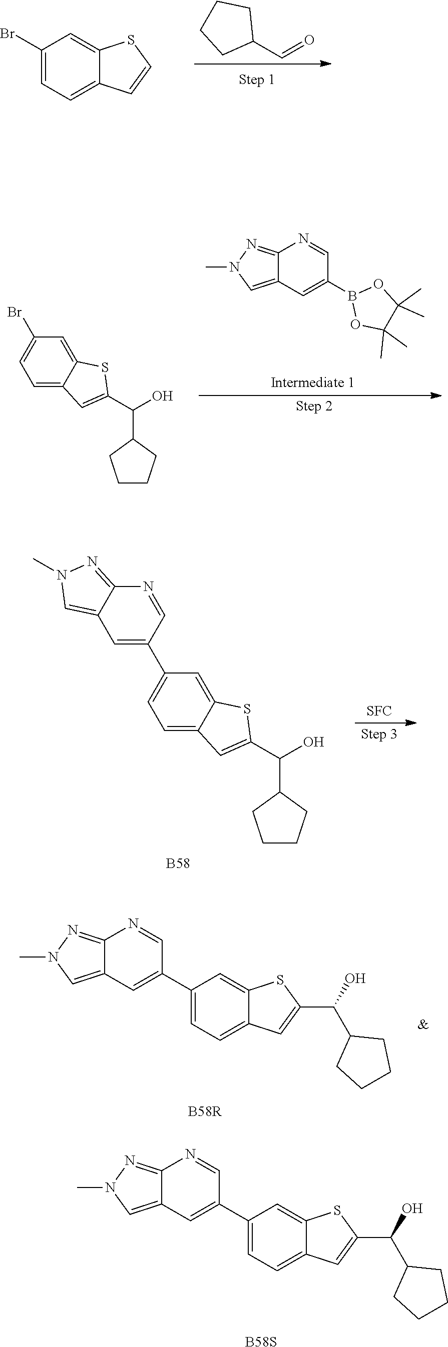

Step 1: (6-bromobenzo[b]thiophen-2-yl)(cyclopentyl)methanol. To a solution of 6-bromobenzo[b]thiophene (500 mg, 2.35 mmol) in THF (1 mL) was added a solution of LDA in THF (2.0 M, 1.29 mL, 2.58 mmol) at −70° C. under N₂. After 60 min, cyclopentanecarbaldehyde (253 mg, 2.58 mmol) was added. The mixture was stirred at −70° C. for a further 2 h. The reaction mixture was quenched by addition of sat. aq. NH₄Cl (40 mL) at 0° C. The mixture was extracted with EtOAc (3×30 mL). The combined organic layers were washed with brine (100 mL), dried over Na₂SO₄, filtered and concentrated under reduced pressure to obtain a crude residue. The residue was purified by chromatography, eluting with a gradient of 0-10% EtOAc in pet. ether, to provide (6-bromobenzo[b]thiophen-2-yl)(cyclopentyl)methanol (600 mg, 1.93 mmol, 82% yield), which was used in the next step directly. m z (ESI): 293.1 (M-OH)⁺.

Step 2: cyclopentyl(6-(2-methyl-2H-pyrazolo[3,4-b]pyridin-5-yl)-1-benzothiophen-2-yl)methanol. A mixture of (6-bromobenzo[b]thiophen-2-yl)(cyclopentyl)methanol (600 mg, 1.93 mmol), Intermediate 1 (599 mg, 2.31 mmol), K₂CO₃ (666 mg, 4.82 mmol), and Pd(dppf)Cl₂ (141 mg, 0.19 mmol) in H₂O (10 mL) and 1,4-dioxane (1 mL) was purged with N₂. The mixture was stirred at 110° C. for 12 h under N₂. The reaction mixture was quenched by addition of H₂O (20 mL) and then extracted with EtOAc (5×20 mL). The combined organic layers were washed with brine (20 mL), dried over Na₂SO₄, filtered, and concentrated under reduced pressure to give a crude material. The crude material was purified by chromatography, eluting with a gradient of 0-10% MeOH in EtOAc, to provide Compound B58 (160 mg, 0.41 mmol, 21% yield). m/z (ESI): 364.1 (M+H)⁺; ¹H NMR (400 MHz, DMSO-d₆) δ 8.98 (d, J 2.0 Hz, 1H), 8.49-8.46 (m, 2H), 8.31 (s, 1H), 7.87 (d, J 8.4 Hz, 1H), 7.84-7.70 (m, 1H), 7.28 (s, 1H), 5.75 (d, J 4.8 Hz, 1H), 4.69-4.64 (m, 1H), 4.22 (s, 3H), 2.24-2.22 (m, 1H), 1.72-1.62 (m, 1H), 1.57-1.51 (m, 6H), 1.49-1.33 (m, 1H).

Step 3: SFC Purification. The sample was purified via SFC using a Chiralpak AD-3, 150×4.6 mm, 3 um, column with a mobile phase of 50% EtOH with 0.1% IPAm using a flowrate of 2.5 mL/min to generate peak 1 and peak 2:

Peak 1:58R. Compound 58R was obtained (58 mg, 0.16 mmol, 38% yield). m/z (ESI): 364.1 (M+H)⁺. ¹H NMR (400 MHz, DMSO-d₆) δ 8.98 (d, J 2.1 Hz, 1H), 8.51-8.43 (m, 2H), 8.31 (s, 1H), 7.86 (d, J 8.4 Hz, 1H), 7.72 (dd, J 1.3, 8.2 Hz, 1H), 7.29 (s, 1H), 5.75 (d, J 4.5 Hz, 1H), 4.67 (dd, J 4.7, 7.2 Hz, 1H), 4.23 (s, 3H), 2.27-2.17 (m, 1H), 1.78-1.68 (m, 1H), 1.60-1.44 (m, 6H), 1.37-1.29 (m, 1H).

Peak 2:58S. Compound 58S was obtained (49.6 mg, 0.13 mmol, 32% yield). m/z (ESI): 364.1 (M+H)⁺. ¹H NMR (400 MHz, DMSO-d₆) δ 8.98 (d, J 2.3 Hz, 1H), 8.51-8.44 (m, 2H), 8.31 (s, 1H), 7.86 (d, J 8.3 Hz,1H), 7.72 (dd, J 1.4, 8.4 Hz, 1H), 7.29 (s, 1H), 5.75 (d, J 4.5 Hz, 1H), 4.67 (dd, J 4.7, 7.3 Hz, 1H), 4.23 (s, 3H), 2.27-2.16 (m, 1H), 1.74 (br d, J 8.0 Hz, 1H), 1.62-1.45 (m, 6H), 1.38-1.28 (m, 1H).

Example 4-2: (R)-(3,3-difluorocyclobutyl)(6-(2-methyl-2H-pyrazolo[3,4-b]pyridin-5-yl)-1-benzothiophen-2-yl)methanol B61R & (S)-(3,3-difluorocyclobutyl)(6-(2-methyl-2H-pyrazolo[3,4-b]pyridin-5-yl)-1-benzothiophen-2-yl)methanol B61S

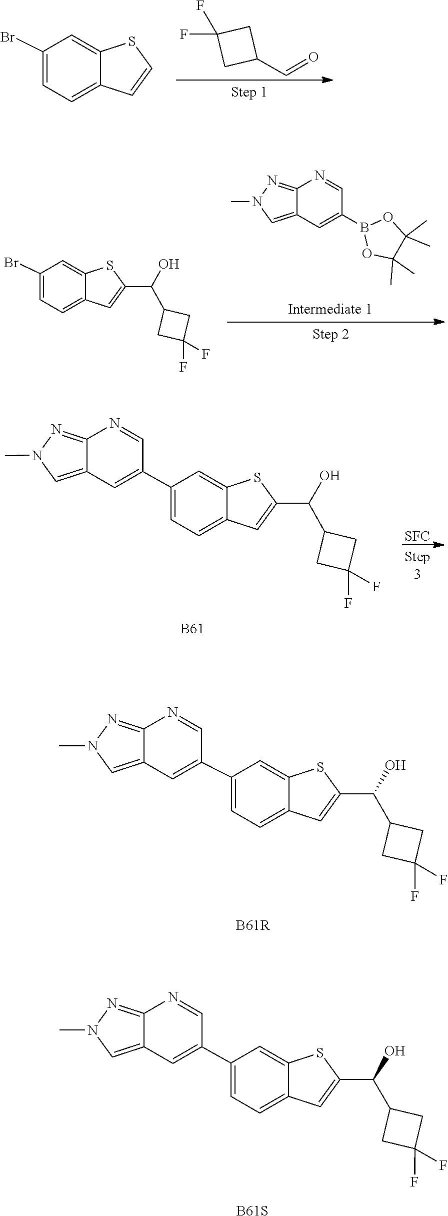

Step 1: (6-bromobenzo[b]thiophen-2-yl)(3,3-difluorocyclobutyl)methanol. To a solution of 6-bromobenzo[b]thiophene (400 mg, 1.88 mmol) in THF (1 mL) was added a solution of LDA in THF (2 M, 1.03 mL, 2.07 mmol) at −70° C. over 60 min under N₂ atmosphere. Then, 3,3-difluorocyclobutane-1-carbaldehyde (248 mg, 2.07 mmol) was added at −70° C., and the reaction mixture was stirred for 1 h. The mixture was then quenched by addition of H₂O (20 mL) and extracted with EtOAc (6×20 mL). The combined organic layers were washed with brine (30 mL), dried over Na₂SO₄, filtered, and concentrated in vacuo. The crude material was absorbed onto a plug of silica gel and purified by column chromatography, eluting with a gradient of 0% to 30% EtOAc in pet ether, to provide (6-bromobenzo[b]thiophen-2-yl)(3,3-difluorocyclobutyl)methanol (290 mg, 0.84 mmol, 45% yield). ¹H NMR (400 MHz, DMSO-d₆) δ 8.20 (d, J 1.6 Hz, 1H), 7.71 (d, J 8.8 Hz, 1H), 7.47 (dd, J 1.8, 8.6 Hz, 1H), 7.30 (s, 1H), 6.18 (d, J 5.2 Hz, 1H), 4.88 (t, J 5.2 Hz, 1H), 2.56-2.52 (m, 5H).

Step 2: (3,3-difluorocyclobutyl)(6-(2-methyl-2H-pyrazolo[3,4-b]pyridin-5-yl)benzo[b]thiophen-2-yl)methanol. A mixture of (6-bromobenzo[b]thiophen-2-yl)(3,3-difluorocyclobutyl)methanol (240 mg, 0.72 mmol), Intermediate 1 (224 mg, 0.86 mmol), K₂CO₃ (249 mg, 1.80 mmol), and Pd(dppf)Cl₂ (52.7 mg, 0.072 mmol) in H₂O (1 mL) and 1,4-dioxane (5 mL) was degassed with N₂. The mixture was stirred at 110° C. for 12 h. The reaction mixture was diluted with water (30 mL) and extracted with EtOAc (50 mL×3). The combined organic layers were washed with brine (50 mL), dried over Na₂SO₄, filtered and concentrated under reduced pressure. The residue was purified by column chromatography, eluting with 0 to 90% EtOAc in pet. ether, to afford Compound B61 (130 mg, 0.337 mmol, 47% yield). m/z (ESI): 386.0 (M+H)⁺. ¹H NMR (400 MHz, DMSO-d₆) δ 8.98 (d, J 2.3 Hz, 1H), 8.53-8.43 (m, 2H), 8.33 (s, 1H), 7.87 (d, J 8.4 Hz, 1H), 7.73 (dd, J 8.3, 1.6 Hz, 1H), 7.34 (s, 1H), 6.17 (br s, 1H), 4.94-4.87 (m, 1H), 4.23 (s, 3H), 2.68-2.52 (m, 5H).

Step 3: SFC Purification. The sample was purified via SFC using a Chiralpak AD-3, 50×4.6 mm I.D., 3 um, column with a mobile phase of 50% EtOH (0.1% IPAm, v/v) with a flow rate of 4 mL/min to generate peak 1 and peak 2.

Peak 1: B61S. Compound B61S was isolated (35 mg, 0.092 mmol, 29% yield). m/z (ESI): 386.0 (M+H)⁺. ¹H NMR (400 MHz, DMSO-d₆) δ 8.98 (s, 1H), 8.55-8.39 (m, 2H), 8.32 (s, 1H), 7.87 (d, J 8.0 Hz, 1H), 7.73 (d, J 8.0 Hz, 1H), 7.34 (s, 1H), 6.16 (d, J 5.2 Hz, 1H), 4.97-4.87 (m, 1H), 4.23 (s, 3H), 2.60-2.55 (m, 5H).

Peak 2: B61R. Compound B61R was isolated (33 mg, 0.085 mmol, 28% yield). m/z (ESI): 386.0 (M+H)⁺. ¹H NMR (400 MHz, DMSO-d₆) δ 8.98 (d, J=2.3 Hz, 1H), 8.55-8.43 (m, 2H), 8.32 (s, 1H), 7.87 (br d, J 8.1 Hz, 1H), 7.73 (br d, J 8.3 Hz, 1H), 7.34 (s, 1H), 6.16 (br d, J 4.9 Hz, 1H), 4.94-4.88 (m, 1H), 4.23 (s, 3H), 2.66-2. 53 (m, 5H). Alternate conditions in reference to Example 4-1:
(1) To a solution of 5-chlorothieno[3,2-b]pyridine (2 g, 11.79 mmol) in THF (20 mL) at −78° C. was added a solution of n-butyllithium in hexanes (2.5 M, 9.43 mL, 23.58 mmol) dropwise over 15 min, and the mixture was stirred for 15 m. Next, 3,3-difluorocyclobutane-)-carbaldehyde (2.83 g, 23.58 mmol) was added dropwise over 5 min at −78° C., and the mixture was stirred for 1 h. The reaction was quenched by addition of satd. aq. NH₄C₁, and extracted with EtOAc (30 mL×2). Then, the extracted layers were washed with brine (10 mL×2), dried over Na₂SO₄, filtered, and concentrated under reduced pressure. The crude residue was purified by chromatography, eluting with a gradient of 30-50% o EtOAc in pet. ether, to provide the product.
(2) 10 mole SPhos Pd G3 was used as the catalyst, instead of Pd(dppf)Cl₂.

Compounds in Table 4-1 were prepared following the procedure described in Group 4 above using appropriate starting materials. All starting materials are commercially available or are described in the Intermediates section above.

TABLE 4-1

| | | | Reaction |
|---|---|---|---|
| Ex. # | Chemical Structure & Name | LCMS: (ESI + ve ion) m/z; NMR | Comments |
| B54 | 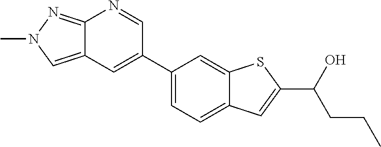 [SMILES: CCCC(O)c1cc2ccc(-c3cnc4nn(C)cc4c3)cc2s1] | 338.1; ¹H NMR (400 MHz, DMSO- d₆) δ 8.98 (d, J = 2.4 Hz, 1H), 8.49- 8.47 (m, 2H), 8.31 (s, 1H), 7.86 (d, J = 8.4 Hz, 1H), 7.72 (dd, J = 8.3, 1.6 Hz, 1H), 7.28 (s, 1H), 5.72 (d, J = 4.8 Hz, 1H), 4.91-4.86 (m, 1H), 4.23 (s, 3H), 1.76-1.72 (m, 2H), 1.42-1.36 (m, 2H), 0.91 (t, J = 7.4 Hz, 3H) | |
| | 1-(6-(2-methyl-2H-pyrazolo[3,4- | | |
| | b]pyridin-5-yl)-1- | | |
| | benzothiophen-2-yl)-1-butanol | | |
| B54R | 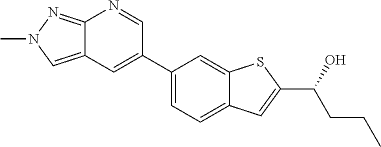 [SMILES: CCC[C@@H](O)c1cc2ccc(-c3cnc4nn(C)cc4c3)cc2s1] | 338.1; ¹H NMR (400 MHz, DMSO- d₆) δ 8.98 (d, J = 2.4 Hz, 1H), 8.53- 8.46 (m, 2H), 8.31 (s, 1H), 7.86- 7.84 (m, 1H), 7.72-7.70 (m, 1H), 7.31-7.25 (m, 1H), 5.76-5.70 (m, 1H), 4.92-4.85 (m, 1H), 4.23 (s, 3H), 1.83-1.67 (m, 2H), 1.42-1.37 (m, 2H), 0.92 - 0.89 (m, 3H) | Peak 1/SFC: Chiralcel OZ, 250 mm × 25 mm, 10 μm; mobile phase: 65% MeOH in CO₂ |
| | (1R)-1-(6-(2-methyl-2H- | | |
| | pyrazolo[3,4-b]pyridin-5-yl)-1- | | |
| | benzothiophen-2-yl)-1-butanol | | |
| B54S | 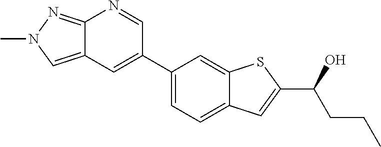 [SMILES: CCC[C@H](O)c1cc2ccc(-c3cnc4nn(C)cc4c3)cc2s1] | 338.1; ¹H NMR (400 MHz, DMSO- d₆) δ 8.98 (d, J = 2.4 Hz, 1H), 8.49- 8.47 (m, 2H), 8.31 (s, 1H), 7.86 (d, J = 8.4 Hz, 1H), 7.72 (dd, J = 8.3, 1.7 Hz, 1H), 7.28 (s, 1H), 5.73 (br d, J = 4.5 Hz, 1H), 4.91-4.86 (m, 1H), 4.23 (s, 3H), 1.76-1.72 (m, 2H), 1.42-1.36 (m, 2H), 0.91 (t, J = 7.4 Hz, 3H) | Peak 2/SFC: Chiralcel OZ, 250 mm × 25 mm, 10 μm; mobile phase: 65% MeOH in CO₂ |
| | (1S)-1-(6-(2-methyl-2H- | | |
| | pyrazolo[3,4-b]pyridin-5-yl)-1- | | |
| | benzothiophen-2-yl)-1-butanol | | |
| B59 | 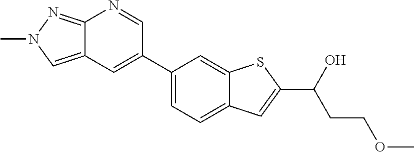 | 354.1; ¹H NMR (400 MHz, DMSO- d₆) δ 8.98 (d, J = 2.4 Hz, 1H), 8.49- 8.44 (m, 2H), 8.32 (s, 1H), 7.87- 7.84 (m, 1H), 7.74-7.71 (m, 1H), 7.28 (s, 1H), 5.83 (d, J = 4.8 Hz, 1H), 5.02-4.96 (m, 1H), 4.22 (s, 3H), 3.52-3.34 (m, 1H), 3.42-3.37 (m, 1H), 3.25 (s, 3H), 2.01-1.96 (m, 2H) | |
| | 3-methoxy-1-(6-(2-methyl-2H- | | |
| | pyrazolo[3,4-b]pyridin-5-yl)-1- | | |
| | benzothiophen-2-yl)-1-propanol | | |
| B59S | 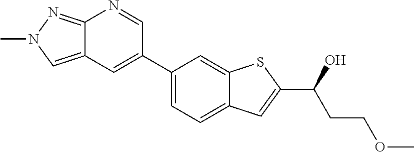 | 354.1; ¹H NMR (400 MHz, DMSO- d₆) δ 8.98-8.96 (m, 1H), 8.49- 8.47 (m, 2H), 8.32 (s, 1H), 7.87- 7.86 (m, 1H), 7.74-7.72 (m, 1H), 7.29 (s, 1H), 5.84 (d, J = 4.0Hz, 1H), 5.02-4.96 (m, 1H), 4.22 (s, 3H), 3.52-3.50 (m, 1H), 3.49-3.35 (m, 1H), 3.25 (s, 3H), 2.01-1.97 (m, 2H) | Peak 1/SFC: Chiralcel OZ-3, 100 × 4.6 mm I.D., 3 μm Mobile phase: 50% CO₂ : 50% EtOH (0.1% IPAm, v/v) using a flow rate of 4 mL/min |
| | (1S)-3-methoxy-1-(6-(2-methyl- | | |
| | 2H-pyrazolo[3,4-b]pyridin-5-y1)- | | |
| | 1-benzothiophen-2-y1)-1- | | |
| | propanol | | |
| B59R | 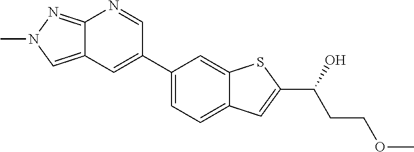 | 354.1; ¹H NMR (400 MHz, DMSO- d₆) δ 8.98 (d, J = 2.3 Hz, 1H), 8.51- 8.46 (m, 2H), 8.32 (d, J = 0.7 Hz, 1H), 7.87 (d, J = 8.2 Hz, 1H), 7.73 (dd, J = 1.7, 8.3 Hz, 1H), 7.29 (s, 1H), 5.84 (d, J = 5.0 Hz, 1H), 5.02- 4.95 (m, 1H), 4.23 (s, 3H), 3.51 (td, J = 6.8, 9.4 Hz, 1H), 3.43-3.36 (m, 1H), 3.25 (s, 3H), 2.02-1.94 (m, 2H) | Peak 2/SFC: Chiralcel OZ-3, 100 × 4.6 mm I.D., 3 μm Mobile phase: 50% CO₂ : 50% EtOH (0.1% IPAm, v/v) using a flow rate of 4 mL/min |
| | (1R)-3-methoxy-1-(6-(2-methyl- | | |
| | 2H-pyrazolo[3,4-b]pyridin-5-yl)- | | |
| | 1-benzothiophen-2-y1)-1- | | |
| | propanol | | |
| B60 | 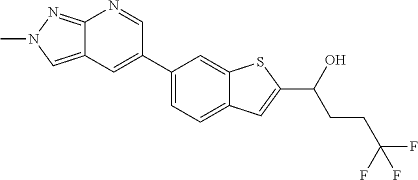 [SMILES: Cn1cc2cc(-c3ccc4cc(C(O)CCC(F)(F)F)sc4c3)cnc2n1] | 392.0; ¹H NMR (400 MHz, DMSO- d₆) δ 8.99 (d, J = 2.4 Hz, 1H), 8.52- 8.46 (m, 2H), 8.34 (s, 1H), 7.89 (d, J = 8.4 Hz, 1H), 7.75-7.73 (m, 1H), 7.34 (s, 1H), 6.09 (d, J = 5.2 Hz, 1H), 5.03-4.99 (m, 1H), 4.23 (s, 3H), 2.45-2.25 (m, 2H), 2.10-1.90 (m, 2H) | |
| | 4,4,4-trifluoro-1-(6-(2-methyl- | | |
| | 2H-pyrazolo[3,4-b]pyridin-5-yl)- | | |
| | 1-benzothiophen-2-y1)-1-butanol | | |
| B60R | 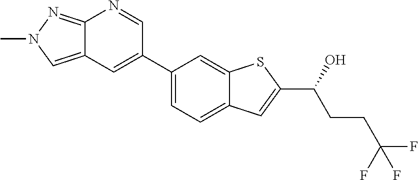 [SMILES: Cn1cc2cc(-c3ccc4cc([C@H](O)CCC(F)(F)F)sc4c3)cnc2n1] | 392.0; ¹H NMR (400 MHz, DMSO- d6) 8 8.98 (d, J = 1.6 Hz, 1H), 8.53 - 8.43 (m, 8.34 (s, 1H), 7.89 (d, J = 8.4 Hz, 1H), 7.74 (d, J = 8.4 Hz, 1H), 7.34 (s, 1H), 6.11 (s, 1H), 5.01 (t, J = 6.4 Hz, 1H), 4.23 (s, 3H), 2.50-2.25 (m, 2H), 2.10-1.85 (m, 2H) | Peak 1/SFC: Chiralpak AD-3, 50 × 4.6 mm I.D., 3 μm, Mobile phase: (5%-95%) MeOH (0.1% IPAm, v/v) in CO₂ using a flow rate of 3.4 mL/min |
| | (1R)-4,4,4-trifluoro-1-(6-(2- | | |
| | methyl-2H-pyrazolo[3,4- | | |
| | b]pyridin-5-yl)-1- | | |
| | benzothiophen-2-yl)-1-butanol | | |
| B60S | 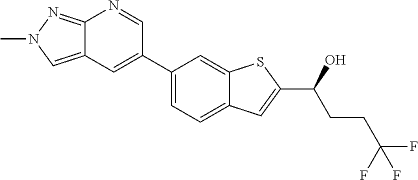 [SMILES: Cn1cc2cc(-c3ccc4cc([C@@H](O)CCC(F)(F)F)sc4c3)cnc2n1] | 392.0; ¹H NMR (400 MHz, DMSO- d₆) δ 8.98 (d, J = 2.0 Hz, 1H), 8.52- 8.46 (m, 2H), 8.34 (d, J = 0.8 Hz, 1H), 7.89 (d, J = 8.0 Hz, 1H), 7.78- 7.70 (m, 1H), 7.35 (s, 1H), 6.09 (s, 1H), 5.01 (t, J = 6.4 Hz, 1H), 4.23 (s, 3H), 2.45-2.25 (m, 2H), 2.05- 1.90 (m, 2H) | Peak 2/SFC: Chiralpak AD-3, 50 × 4.6 mm I.D., 3 μm, Mobile phase: (5%-95%) MeOH (0.1% IPAm, v/v) in CO₂ using a flow rate of 3.4 mL/min |
| | (1S)-4,4,4-trifluoro-1-(6-(2- | | |
| | methyl-2H-pyrazolo[3,4- | | |
| | b]pyridin-5-y1)-1- | | |
| | benzothiophen-2-yl)-1-butanol | | |
| B73R | 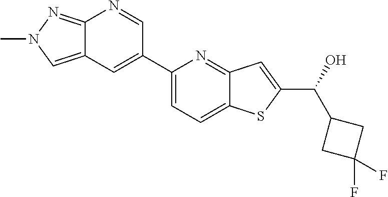 [SMILES: Cn1cc2cc(-c3ccc4sc([C@H](O)C5CC(F)(F)C5)cc4n3)cnc2n1] | 387.1; ¹H NMR (401 MHz, DMSO- d₆) δ 9.40 (d, J = 2.3 Hz, 1H), 8.89 (d, J = 2.3 Hz, 1H), 8.56-8.45 (m, 2H), 8.00 (d, J = 8.6 Hz, 1H), 7.51 (s, 1H), 6.31 (d, J = 5.1 Hz, 1H), 5.00 (d, J = 4.8 Hz, 1H), 4.24 (s, 3H), 2.70-2.55 (m, 5H) | Alternate condition (1) was used; Peak 1/SFC: Chiralpak AS-H 250 × 30 mm, 5 μm; Mobile phase: 30% MeOH in CO₂ with a flow rate of 130 mL/min |
| | (R)-(3,3-difluorocyclobutyl)(5- | | |
| | (2-methyl-2H-pyrazolo[3,4- | | |
| | b]pyridin-5-y1)thieno[3,2- | | |
| | b]pyridin-2-yl)methanol | | |
| B73S | 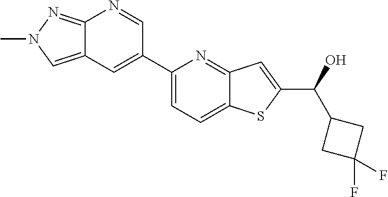 [SMILES: Cn1cc2cc(-c3ccc4sc([C@@H](O)C5CC(F)(F)C5)cc4n3)cnc2n1] | 387.0; ¹H NMR (401 MHz, DMSO- d₆) δ 9.40 (d, J = 2.3 Hz, 1H), 8.89 (d, J = 2.3 Hz, 1H), 8.62-8.44 (m, 2H), 8.00 (d, J = 8.5 Hz, 1H), 7.51 (s, 1H), 6.31 (d, J = 5.1 Hz, 1H), 5.00 (d, J = 4.8 Hz, 1H), 4.24 (s, 3H), 2.74-2.56 (m, 5H) | Alternate condition (1) was used; Peak 2/SFC: Chiralpak AS-H 250 × 30 mm, 5 μm; Mobile phase: 30% MeOH in CO₂ with a flow rate of 130 mL/min |
| | (S)-(3,3-difluorocyclobutyl)(5- | | |
| | (2-methyl-2H-pyrazolo[3,4- | | |
| | b]pyridin-5-yl)thieno[3,2- | | |
| | b]pyridin-2-yl)methanol | | |
| C84C | 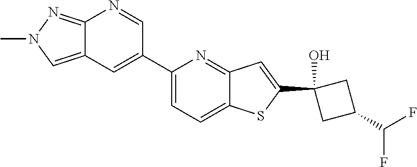 [SMILES: Cn1cc2cc(-c3ccc4sc([C@]5(O)C[C@@H](C(F)F)C5)cc4n3)cnc2n1] | 387.0; ¹H NMR (401 MHz, DMSO- d₆) δ 9.42 (d, J = 2.3 Hz, 1H), 8.89 (d, J = 2.3 Hz, 1H), 8.55 (s, 1H), 8.50 (d, J = 8.5 Hz, 1H), 8.01 (d, J = 8.5 Hz, 1H), 7.68 (s, 1H), 6.52 (s, 1H), 6.17 (d, J = 57.2, 4.2 Hz, 1H), 4.25 (s, 3H), 2.64 (td, J = 9.2, 3.2 Hz, 3H), 2.51-2.41 (m, 2H) | Alternate conditions (1) and (2) were used; Step 3 was not performed; |
| | cis-3-(difluoromethyl)-1-(5-(2- | | |
| | methyl-2H-pyrazolo[3,4- | | |
| | b]pyridin-5-y1)thieno[3,2- | | |
| | b]pyridin-2-yl)cyclobutanol | | |
| C86C | 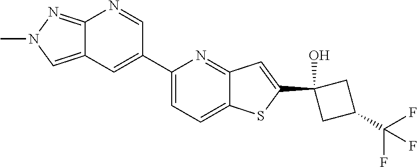 [SMILES: Cn1cc2cc(-c3ccc4sc([C@]5(O)C[C@@H](C(F)(F)F)C5)cc4n3)cnc2n1] | 405.2; ¹H NMR (401 MHz, DMSO- d₆) δ 9.42 (d, J = 2.3 Hz, 1H), 8.90 (d, J = 2.3 Hz, 1H), 8.56 (s, 1H), 8.51 (d, J = 8.5 Hz, 1H), 8.02 (d, J = 8.5 Hz, 1H), 7.78 (s, 1H), 6.72 (s, 1H), 4.25 (s, 3H), 3.20 (dt, J = 18.2, 9.0 Hz, 1H), 2.86-2.73 (m, 2H), 2.57 (dd, J = 12.5, 9.4 Hz, 2H) | Alternate conditions (1) and (2) were used; Step 3 was not performed; |
| | cis-1-(5-(2-methyl-2H- | | |
| | pyrazolo[3,4-b]pyridin-5- | | |
| | yl)thieno[3,2-b]pyridin-2-yl)-3- | | |
| | (trifluoromethyl)cyclobutanol | | |
| C106T | 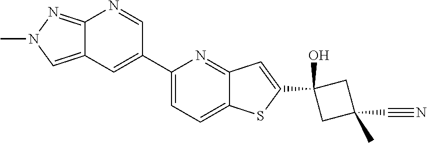 [SMILES: Cn1cc2cc(-c3ccc4sc([C@]5(O)C[C@](C)(C#N)C5)cc4n3)cnc2n1] | 376.2; ¹H NMR (401 MHz, DMSO- d₆) δ 9.41 (d, J = 2.3 Hz, 1H), 8.92 (d, J = 2.3 Hz, 1H), 8.57-8.49 (m, 2H), 8.03 (d, J = 8.6 Hz, 1H), 7.72 (d, J = 0.7 Hz, 1H), 6.67 (s, 1H), 4.25 (s, 3H), 3.23-3.15 (m, 2H), 2.62 (d, J = 2.7 Hz, 1H), 2.62-2.52 (m, 1H), 1.68 (s, 3H) | Alternate condition (1) was used; After Step 1, the sample was purified by SFC Peak 1/SFC: Cellulose SZ 250 × 4.6 mm, 5 μm column; |
| | | | Mobile phase: 50% |
| | trans-3-hydroxy-1-methyl-3-(5- | | (1:1) MeOH:ACN |
| | (2-methyl-2H-pyrazolo[3,4- | | in CO₂ and a flow |
| | b]pyridin-5-yl)thieno[3,2- | | rate of 120 mL/min |
| | b]pyridin-2- | | |
| | yl)cyclobutanecarbonitrile | | |
| C106C | 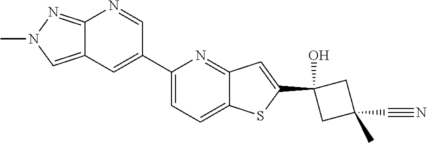 [SMILES: Cn1cc2cc(-c3ccc4sc([C@]5(O)C[C@@](C)(C#N)C5)cc4n3)cnc2n1] | 376.2; ¹H NMR (401 MHz, DMSO- d₆) δ 9.41 (d, J = 2.3 Hz, 1H), 8.89 (d, J = 2.3 Hz, 1H), 8.58-8.48 (m, 2H), 8.02 (d, J = 8.6 Hz, 1H), 7.69 (d, J = 0.7 Hz, 1H), 6.72 (s, 1H), 4.24 (s, 3H), 2.93-2.84 (m, 2H), 2.84-2.72 (m, 2H), 1.57 (s, 3H) | Alternate condition (1) was used; After Step 1, the sample was purified by SFC Peak 2/SFC: Cellulose SZ 250 × 4.6 mm, 5 μm column; |
| | | | Mobile phase: 50% |
| | cis-3-hydroxy-1-methyl-3-(5-(2- | | (1:1) MeOH:ACN |
| | methyl-2H-pyrazolo[3,4- | | in CO₂ and a flow |
| | b]pyridin-5-yl)thieno[3,2- | | rate of 120 mL/min |
| | b]pyridin-2- | | |
| | yl)cyclobutanecarbonitrile | | |
| C127C | 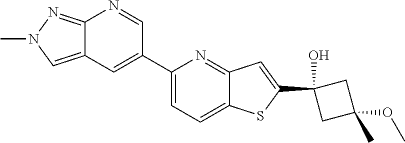 | 381.2; ¹H NMR (400 MHz, DMSO- d₆) δ 9.41 (d, J = 2.3 Hz, 1H), 8.90 (d, J = 2.3 Hz, 1H), 8.55 (s, 1H), 8.49 (d, J = 8.5 Hz, 1H), 8.01 (d, J = 8.6 Hz, 1H), 7.57 (s, 1H), 6.26 (s, 1H), 4.25 (s, 3H), 3.15 (s, 3H), 2.72- 2.64 (m, 2H), 2.62-2.54 (m, 2H), 1.27 (s, 3H). | Alternate condition (2) was used |
| | cis-3-methoxy-3-methyl-1-(5-(2- | | |
| | methyl-2H-pyrazolo[3,4- | | |
| | b]pyridin-5-y1)thieno[3,2- | | |
| | b]pyridin-2-yl)cyclobutanol | | |

### Group 5

Example 5-1: (6-(2-methyl-2H-pyrazolo[3,4-b]pyridin-5-yl)thieno[2,3-b]pyridin-2-yl)methanol A1

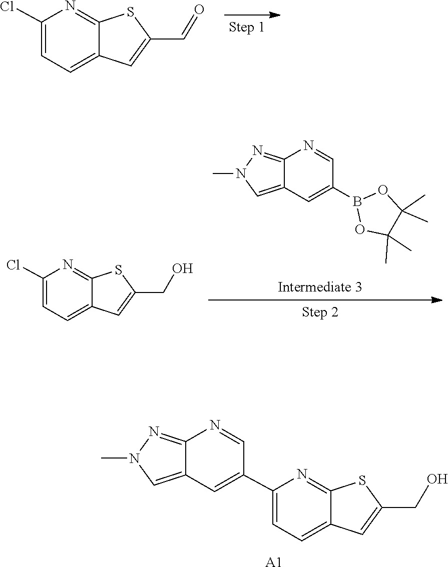

Step 1: (6-chlorothieno[2,3-b]pyridin-2-yl)methanol. To a stirred mixture of 6-chlorothieno[2,3-b]pyridine-2-carbaldehyde (210 mg, 1.06 mmol) in MeOH (2.5 mL) was added NaBH₄ (44.2 mg, 1.17 mmol) at 0° C., and the reaction was stirred for 0.5 h. The reaction mixture was quenched by addition of sat. aq. NH₄Cl (20 mL) at 25° C., extracted with EtOAc (20 mL×4), washed with brine (20 mL), dried over Na₂SO₄, filtered and concentrated in vacuo. The crude material was absorbed onto a plug of silica gel and purified by column chromatography, eluting with a gradient of 0% to 28% EtOAc in pet ether, to provide (6-chlorothieno[2,3-b]pyridin-2-yl)methanol (170 mg, 0.85 mmol, 80% yield). m z (ESI): 199.9 (M+H)⁺. ¹H NMR (400 MHz, DMSO-d₆) δ 8.22 (d, J 8.4 Hz, 1H), 7.48 (d, J 8.3 Hz, 1H), 7.30 (s, 1H), 5.81 (t, J 5.77 Hz, 1H), 4.77 (dd, J 5.77, 1.00 Hz, 2H).

Step 2: (6-(2-methyl-2H-pyrazolo[3,4-b]pyridin-5-yl)thieno[2,3-b]pyridin-2-yl)methanol. A mixture of (6-chlorothieno[2,3-b]pyridin-2-yl)methanol (170 mg, 0.85 mmol), Intermediate 1 (441 mg, 1.70 mmol), K₂CO₃ (353 mg, 2.55 mmol), and Pd(dppf)Cl₂ (31.2 mg, 0.043 mmol) in 1,4-dioxane (1.6 mL) and H₂O (0.2 mL) was stirred at 110° C. for 2 h. The reaction was quenched by addition of H₂O (80 mL), extracted with EtOAc:MeOH (10:1; 8×30 mL), dried over Na₂SO₄, filtered and concentrated. The residue was purified by prep-HPLC, eluting with a gradient of 15%-45% MeCN in H₂O (10 mM NH₄HCO₃), to provide Compound A1 (47.7 mg, 0.16 mmol, 19% yield). m/z (ESI): 297.1 (M+H)⁺. ¹H NMR (400 MHz, DMSO-d₆) δ 9.38 (d, J 2.1 Hz, 1H), 8.91 (d, J 2.2 Hz, 1H), 8.53 (s, 1H), 8.26 (d, J 8.4 Hz, 1H), 8.08 (d, J 8.3 Hz, 1H), 7.29 (s, 1H), 5.78 (t, J 5.8 Hz, 1H), 4.79 (d, J 5.6 Hz, 2H), 4.24 (s, 3H).

### Group 6

Example 6-1: (S)-(6-(2-methyl-2H-pyrazolo[3,4-b]pyridin-5-yl)thieno[2,3-b]pyridin-2-yl)(tetrahydro-2H-pyran-4-yl)methan-d-ol B66S; (R)-(6-(2-Methyl-2H-pyrazolo[3,4-b]pyridin-5-yl)thieno[2,3-b]pyridin-2-yl)(tetrahydro-2H-pyran-4-yl)methan-d-ol B66R

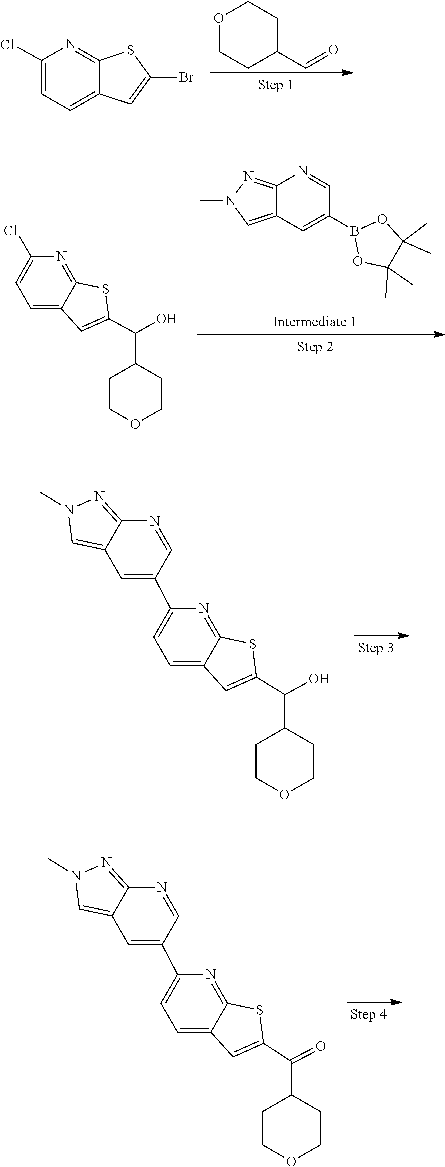

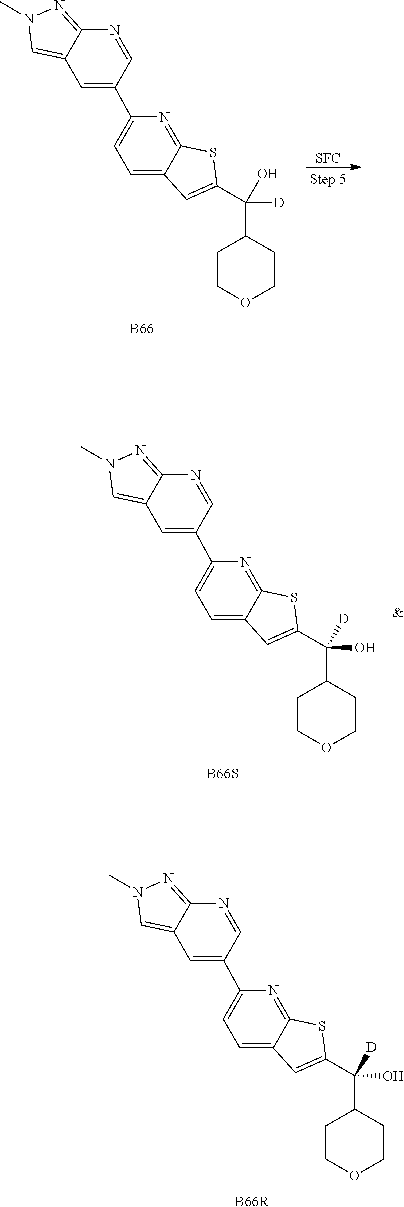

Step 1: (6-chlorothieno[2,3-b]pyridin-2-yl)(tetrahydro-2H-pyran-4-yl)methanol. To a solution of 2-bromo-6-chlorothieno[2,3-b]pyridine (500 mg, 2.01 mmol) in THF (3 mL) was added a solution of n-butyllithium in hexanes (2.5 M, 1.20 mL, 3.02 mmol) and tetrahydro-2H-pyran-4-carbaldehyde (253 mg, 2.21 mmol) at −78° C., and the mixture was stirred for 4 h. The reaction mixture was quenched by addition of H₂O (100 mL) at 0° C., diluted with additional H₂O (30 mL), and extracted with EtOAc (3×80 mL). The combined organic phases were washed with brine (150 mL), dried over Na₂SO₄, filtered and concentrated. The crude material was absorbed onto a plug of silica gel and purified by column chromatography, eluting with a gradient of 0%-50% EtOAc in pet. ether, to provide (6-chlorothieno[2,3-b]pyridin-2-yl)(tetrahydro-2H-pyran-4-yl)methanol (0.571 g, 2.01 mmol, 100% yield) m/z (ESI): 283.9 (M+H)⁺.

Step 2:. (6-(2-methyl-2H-pyrazolo[3,4-b]pyridin-5-yl)thieno[2,3-b]pyridin-2-yl)(tetrahydro-2H-pyran-4-yl)methanol To a microwave tube was added Pd(dppf)Cl₂ (103 mg, 0.14 mmol), K₂CO₃ (390 mg, 2.82 mmol), (6-chlorothieno[2,3-b]pyridin-2-yl)(tetrahydro-2H-pyran-4-yl)methanol (400 mg, 1.41 mmol) and Intermediate 1 (365 mg, 1.41 mmol) in 1,4-dioxane (0.5 mL) and H₂O (0.1 mL). The sealed tube was heated at 120° C. for 1 h in a microwave reactor. The reaction mixture was quenched by addition of H₂O (20 mL) and extracted with EtOAc (3×20 mL). The combined organic phases were washed with brine (15 mL), dried over Na₂SO₄, filtered and concentrated. The crude material was absorbed onto a plug of silica gel and purified by column chromatography, eluting with a gradient of 0% -100% EtOAc in pet ether, to provide (6-(2-methyl-2H-pyrazolo[3,4-b]pyridin-5-yl)thieno[2,3-b]pyridin-2-yl)(tetrahydro-2H-pyran-4-yl)methanol (1.00 g, 2.63 mmol, 75% yield) m/z (ESI): 381.2 (M+H)⁺.

Step 3: (6-(2-methyl-2H-pyrazolo[3,4-b]pyridin-5-yl)thieno[2,3-b]pyridin-2-yl)(tetrahydro-2H-pyran-4-yl)methanone. To a solution of (6-(2-methyl-2H-pyrazolo[3,4-b]pyridin-5-yl)thieno[2,3-b]pyridin-2-yl)(tetrahydro-2H-pyran-4-yl)methanol (600 mg, 1.58 mmol) in DCM (4 mL) and THF (0.6 mL) was added Dess-Martin periodinane (669 mg, 1.58 mmol). The reaction was maintained at 20° C. for 1 h. The reaction mixture was concentrated, and the crude material was purified by prep-HPLC, eluting with a gradient of 25% to 55% MeCN in H₂O (10 mM NH₄HCO₃), to provide (6-(2-methyl-2H-pyrazolo[3,4-b]pyridin-5-yl)thieno[2,3-b]pyridin-2-yl)(tetrahydro-2H-pyran-4-yl)methanone (134.5 mg, 0.35 mmol, 13% yield). m/z (ESI): 379.0 (M+H)⁺.

Step 4: (6-(2-methyl-2H-pyrazolo[3,4-b]pyridin-5-yl)thieno[2,3-b]pyridin-2-yl)(tetrahydro-2H-pyran-4-yl)methan-d-ol. To a solution of (6-(2-methyl-2H-pyrazolo[3,4-b]pyridin-5-yl)thieno[2,3-b]pyridin-2-yl)(tetrahydro-2H-pyran-4-yl)methanone (100 mg, 0.26 mmol) in MeOH (0.5 mL) was added NaBD₄ (16.6 mg, 0.40 mmol) at 0° C. The mixture was stirred at 20° C. for 2 h. The reaction mixture was concentrated and purified by prep-HPLC, eluting with a gradient of 15%-45% MeCN in H₂O (10 mM NH₄HCO₃), to provide Compound B66 (68.5 mg, 0.18 mmol, 68% yield). m z (ESI): 382.1 (M+H)⁺. ¹H NMR (400 MHz, DMSO-d₆) δ 9.37 (d, J 2.4 Hz, 1H), 8.90 (d, J 2.4 Hz, 1H), 8.54 (s, 1H), 8.25 (d, J 8.4 Hz, 1H), 8.08 (d, J 8.4 Hz, 1H), 7.27 (s, 1H), 5.94 (s, 1H), 4.23 (s, 3H), 3.93-3.80 (m, 2H), 3.41-3.25 (m, 2H), 1.84-1.72 (m, 2H), 1.43-1.26 (m, 3H).

Step 5: SFC Separation. Compound B66 was separated into B66R and B66S via SFC using a Chiralpak IH-3, 50×4.6 mm, 3 μm column, with a mobile phase gradient of 5% to 50% EtOH (0.1% IPAm, v/v) and a flow rate of 3.4 mL/min.

Peak 1: B66S. Compound B66S was isolated as the 1^{st} eluting peak (15 mg, 0.040 mmol, 38% yield). m/z (ESI): 382.1 (M+H)⁺. ¹H NMR (400 MHz, DMSO-d₆) δ 9.37 (d, J 2.4 Hz, 1H), 8.90 (d, J 2.4 Hz, 1H), 8.54 (s, 1H), 8.25 (d, J 8.0 Hz, 1H), 8.08 (d, J 8.4 Hz, 1H), 7.27 (s, 1H), 5.94 (s, 1H), 4.24 (s, 3H), 3.93-3.78 (m, 2H), 3.41-3.18 (m, 2H), 1.86-1.72 (m, 2H), 1.41-1.29 (m, 3H).

Peak 2: B66R. Compound B66R was isolated as the 2^{nd} eluting peak (9 mg, 0.023 mmol, 22% yield). m/z (ESI): 382.1(M+H)⁺. ¹H NMR (400 MHz, DMSO-d₆) δ 9.37 (d, J 2.3 Hz, 1H), 8.90 (d, J 2.4 Hz, 1H), 8.54 (s, 1H), 8.25 (d, J 8.4 Hz, 1H), 8.08 (d, J 8.4 Hz, 1H), 7.27 (s, 1H), 5.93 (s, 1H), 4.24 (s, 3H), 3.94-3.78 (m, 2H), 3.29-3.20 (m, 2H), 1.89-1.71 (m, 2H), 1.44-1.22 (m, 3H).

Example 6-2: (R)-(3,3-difluorocyclobutyl)(6-(2-methyl-2H-pyrazolo[3,4-b]pyridin-5-yl)thieno[2,3-b]pyridin-2-yl)methan-d-ol B79R; (S)-(3,3-difluorocyclobutyl)(6-(2-methyl-2H-pyrazolo[3,4-b]pyridin-5-yl)thieno[2,3-b]pyridin-2-yl)methan-d-ol B79S

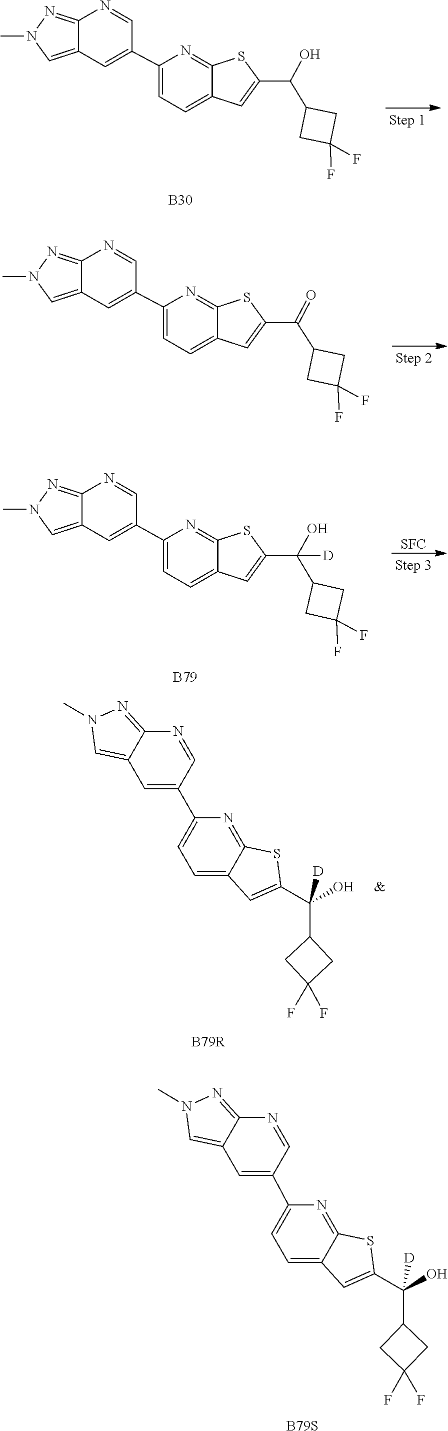

Step 1: (3,3-difluorocyclobutyl)(6-(2-methyl-2H-pyrazolo[3,4-b]pyridin-5-yl)thieno[2,3-b]pyridin-2-yl)methanone. Dess-Martin periodinane (1.09 g, 2.57 mmol) was added to a stirred mixture of sodium bicarbonate (539 mg, 6.42 mmol) and (S)-(3,3-difluorocyclobutyl)(6-(2-methyl-2H-pyrazolo[3,4-b]pyridin-5-yl)thieno[2,3-b]pyridin-2-yl)methanol (496 mg, 1.28 mmol) in dichloromethane (10 mL). The reaction mixture was stirred at rt for 4 h. The reaction mixture was diluted with satd. aq. sodium bicarbonate (75 mL) and washed with DCM (2×100 mL). The aqueous layer was separated, and the suspension was filtered and washed with water to give (3,3-difluorocyclobutyl)(6-(2-methyl-2H-pyrazolo[3,4-b]pyridin-5-yl)thieno[2,3-b]pyridin-2-yl)methanone (213 mg, 0.554 mmol, 43% yield). m z (ESI): 385.0 (M+H)⁺.

Step 2: (3,3-difluorocyclobutyl)(6-(2-methyl-2H-pyrazolo[3,4-b]pyridin-5-yl)thieno[2,3-b]pyridin-2-yl)methan-d-ol. Sodium borodeuteride (49.7 mg, 1.186 mmol) was added to a stirred mixture of (3,3-difluorocyclobutyl)(6-(2-methyl-2H-pyrazolo[3,4-b]pyridin-5-yl)thieno[2,3-b]pyridin-2-yl)methanone (228 mg, 0.59 mmol) in THF (2 mL). The reaction mixture was stirred at rt for 2 h. The reaction mixture was diluted with EtOAc (50 mL) and quenched by addition of satd. aq. NaHCO₃ (30 mL). The organic layer was separated, washed with brine (30 mL), dried over MgSO₄, filtered, and concentrated. The crude material was purified by chromatography, eluting with a gradient of 0 to 100% (3:1) EtOAc:EtOH in heptane to give Compound B79 (92 mg, 0.24 mmol, 40% yield). m/z (ESI): 388.0 (M+H)⁺.

Step 3: SFC Separation. B79 was purified via SFC using a Chiralcel OX, 250×20 mm, 3 μm column, with a mobile phase of 50% MeOH using a flowrate of 80 mL/min., to generate peak 1 and peak 2.

Peak 1: B79R. Compound B79R was isolated as the 1^{st} eluting peak (37 mg). m/z (ESI): 388.0 (M+H)⁺. ¹H NMR (400 MHz, DMSO-d₆) δ 9.38 (d, J=2.1 Hz, 1H), 8.91 (d, J=2.3 Hz, 1H), 8.54 (s, 1H), 8.26 (d, J=8.4 Hz, 1H), 8.09 (d, J=8.6 Hz, 1H), 7.34 (s, 1H), 6.20 (s, 1H), 4.25 (s, 3H), 2.69-2.54 (m, 5H).

Peak 2: B79S. Compound B79S was isolated as the 2^{nd} eluting peak (38 mg). m/z (ESI): 388.0 (M+H)⁺. ¹H NMR (400 MHz, DMSO-d₆) δ 9.38 (d, J=2.1 Hz, 1H), 8.91 (d, J=2.3 Hz, 1H), 8.54 (s, 1H), 8.26 (d, J=8.4 Hz, 1H), 8.09 (d, J=8.4 Hz, 1H), 7.34 (s, 1H), 6.20 (s, 1H), 4.25 (s, 3H), 2.70-2.53 (m, 5H).

### Group 7

Example 7-1: cyclobutyl(6-(2-methyl-2H-pyrazolo[3,4-b]pyridin-5-yl)-4-(1-methyl-1H-pyrazol-5-yl)thieno[2,3-b]pyridin-2-yl)methanol B2

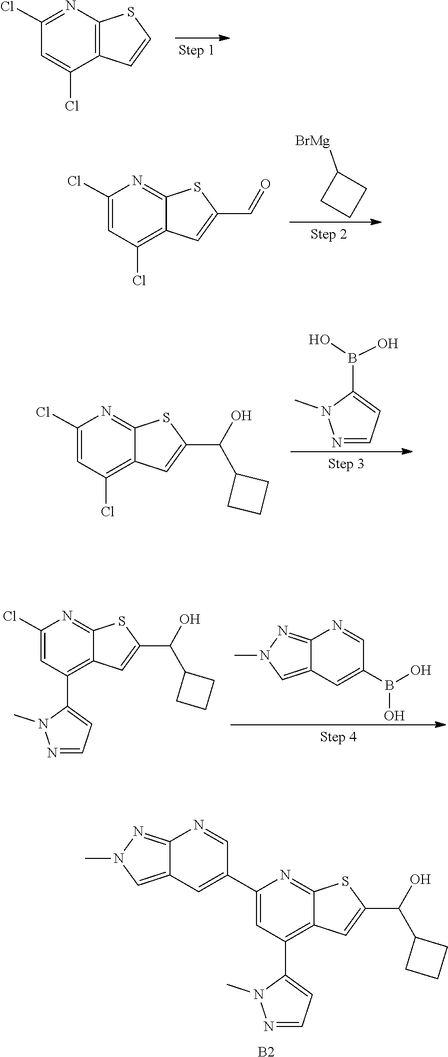

Step 1: 4,6-dichlorothieno[2,3-b]pyridine-2-carbaldehyde. To a stirred solution of 4,6-dichlorothieno[2,3-b]pyridine (2.00 g, 9.80 mmol) in THF (20 mL) was added a solution of LDA in THF (1.0 M, 11.8 mL, 11.8 mmol) at −78° C. under an atmosphere of N₂. The reaction mixture was stirred for 25 min. Then, DMF (0.79 g, 0.84 mL, 10.8 mmol) was transferred at −78° C. The reaction was stirred at -78° C. for a further 20 min, then quenched by addition of sat. aq. NH₄Cl (4 mL). The mixture was warmed to rt and concentrated. The crude material was purified by column chromatography, eluting with a gradient of 0% to 20% EtOAc in heptane, to provide 4,6-dichlorothieno[2,3-b]pyridine-2-carbaldehyde (1.60 g, 6.89 mmol, 70% yield). m/z (ESI): 232.0 (M+H)⁺. ¹H NMR (CHLOROFORM-d, 400 MHz) δ 10.12 (s, 1H), 8.08 (s, 1H), 7.48 (s, 1H).

Step 2: cyclobutyl(4,6-dichlorothieno[2,3-b]pyridin-2-yl)methanol. To a stirred solution of 4,6-dichlorothieno[2,3-b]pyridine-2-carbaldehyde (123 mg, 0.53 mmol) in THF (2.5 mL) was added a solution of cyclobutylmagnesium bromide in THF (0.5 M, 1.06 mL, 0.53 mmol) at 0° C. under an atmosphere of N₂. After 25 min, the reaction mixture was quenched by addition of sat. aq. NH₄Cl and concentrated in vacuo. The crude material was purified by column chromatography, eluting with a gradient of 0% to 4% MeOH in DCM, to provide cyclobutyl(4,6-dichlorothieno[2,3-b]pyridin-2-yl)methanol (145 mg, 0.50 mmol, 95% yield). m/z (ESI): 288.0 (M+H)⁺.

Step 3: (6-chloro-4-(1-methyl-1H-pyrazol-5-yl)thieno[2,3-b]pyridin-2-yl)(cyclobutyl)methanol. A mixture of cyclobutyl(4,6-dichlorothieno[2,3-b]pyridin-2-yl)methanol (145 mg, 0.50 mmol), (1-methyl-1H-pyrazol-5-yl)boronic acid (76 mg, 0.60 mmol), cataCXium A Pd G3 (36.6 mg, 0.050 mmol) and K₃PO₄ monohydrate (348 mg, 1.51 mmol) in THF (10 mL) and H₂O (1 mL) in a microwave reaction vial was purged with N₂ for 15 min. The vial was capped and subjected to microwave irradiation at 70° C. for 1 h. The volatiles were removed, and the crude material was purified by column chromatography, eluting with a gradient of 0% to 11% MeOH in DCM, to provide (6-chloro-4-(1-methyl-1H-pyrazol-5-yl)thieno[2,3-b]pyridin-2-yl)(cyclobutyl)methanol. m/z (ESI): 334.2 (M+H)⁺.

Step 4: cyclobutyl(4-(1-methyl-1H-pyrazol-5-yl)-6-(2-methyl-2H-pyrazolo[3,4-b]pyridin-5-yl)thieno[2,3-b]pyridin-2-yl)methanol. A mixture of (6-chloro-4-(1-methyl-1H-pyrazol-5-yl)thieno[2,3-b]pyridin-2-yl)(cyclobutyl)methanol (85.0 mg, 0.26 mmol), (2-methyl-2H-pyrazolo[3,4-b]pyridin-5-yl)boronic acid (72.1 mg, 0.41 mmol), Pd(dppf)Cl₂ (27.9 mg, 0.038 mmol), and K₂CO₃ (106 mg, 0.76 mmol) in 1,4-dioxane (3 mL) and H₂O (1 mL) in a microwave reactor vial was purged with N₂. The vial was capped and subjected to microwave irradiation at 110° C. for 1 h. The volatiles were removed, and the crude material was purified by column chromatography, eluting with a gradient of 0% to 20% MeOH in DCM, to provide Compound B2 (66 mg, 0.15 mmol, 60% yield). m/z (ESI): 431.2 (M+H)⁺. ¹H NMR (CHLOROFORM-d, 400 MHz) δ 9.36 (d, J 2.1 Hz, 1H), 8.74 (d, J 2.3 Hz, 1H), 8.01 (s, 1H), 7.68 (s, 1H), 7.65 (d, J 1.9 Hz, 1H), 7.01 (s, 1H), 6.50 (d, J 1.9 Hz, 1H), 4.91 (br d, J 7.3 Hz, 1H), 4.29 (s, 3H), 3.87 (s, 3H), 2.84-2.72 (m, 1H), 2.20-1.84 (m, 6H).

Example 7-2: cyclobutyl(4-cyclopropyl-6-(2-methyl-2H-pyrazolo[3,4-b]pyridin-5-yl)thieno[2,3-b]pyridin-2-yl)methanol B8

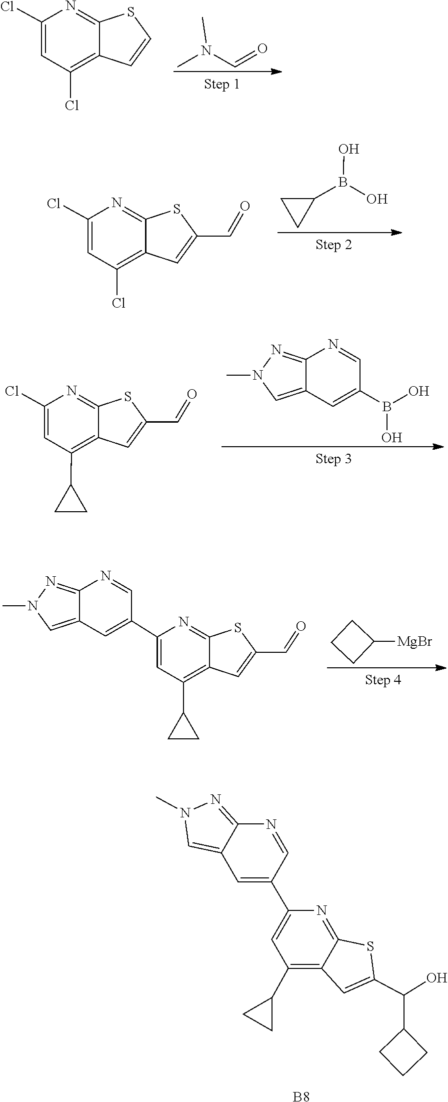

Step 1: 4,6-dichlorothieno[2,3-b]pyridine-2-carbaldehyde. To a stirred solution of 4,6-dichlorothieno[2,3-b]pyridine (2.00 g, 9.80 mmol) in THF (20 mL) at −78° C. was added under N₂ a solution of LDA in THF (1.0 M, 11.8 mL, 11.8 mmol). The resulting mixture was stirred at −78° C. for 25 min before DMF (0.79 g, 0.84 mL, 10.8 mmol) was added via syringe. The resulting mixture was stirred at −78° C. for 18 min, then quenched by addition of satd. aq. NH₄Cl (4 mL). The mixture was allowed to warm to RT. The volatiles were removed, and the crude residue was purified by column chromatography, eluting with a gradient of 0 to 20% MeOH in DCM, to give 4,6-dichlorothieno[2,3-b]pyridine-2-carbaldehyde (1.60 g, 6.89 mmol, 70% yield). m/z (ESI): 232.0 (M+H)⁺. ¹H NMR (CHLOROFORM-d, 400 MHz) δ 10.12 (s, 1H), 8.08 (s, 1H), 7.48 (s, 1H).

Step 2: 6-chloro-4-cyclopropylthieno[2,3-b]pyridine-2-carbaldehyde. A mixture of 4,6-dichlorothieno[2,3-b]pyridine-2-carbaldehyde (300 mg, 1.29 mmol), cyclopropylboronic acid (111 mg, 1.29 mmol), palladium(II) acetate (29.0 mg, 0.13 mmol), PCy₃ (72.5 mg, 0.26 mmol), and K₃PO₄ monohydrate (1.042 g, 4.52 mmol) in toluene (10 mL) and H₂O (1 mL) in a 20 mL microwave reaction vessel was purged with N₂ for 6 min. The vessel was capped and subjected to microwave irradiation at 95° C. for 3 h. The crude mixture was purified by column chromatography, eluting with 0 to 9% MeOH in DCM, to give 6-chloro-4-cyclopropylthieno[2,3-b]pyridine-2-carbaldehyde (105 mg, 0.44 mmol, 34% yield). m/z (ESI): 238.0 (M+H)⁺. ¹H NMR (CHLOROFORM-d, 400 MHz) δ 10.12 (s, 1H), 8.18 (s, 1H), 6.86 (s, 1H), 2.33 (tt, J 5.0, 8.4 Hz, 1H), 1.34-1.30 (m, 2H), 1.03-0.99 (m, 2H). A additional mixture of 6-chloro-4-cyclopropylthieno[2,3-b]pyridine-2-carbaldehyde with 4-chloro-6-cyclopropylthieno[2,3-b]pyridine-2-carbaldehyde (60 mg) was also isolated and taken on in Step 3.

Step 3: 4-cyclopropyl-6-(2-methyl-2H-pyrazolo[3,4-b]pyridin-5-yl)thieno[2,3-b]pyridine-2-carbaldehyde. A mixture of 6-chloro-4-cyclopropylthieno[2,3-b]pyridine-2-carbaldehyde with 4-chloro-6-cyclopropylthieno[2,3-b]pyridine-2-carbaldehyde (60.0 mg, 0.25 mmol), (2-methyl-2H-pyrazolo[3,4-b]pyridin-5-yl)boronic acid (71.5 mg, 0.40 mmol), Pd(dppf)Cl₂ (27.7 mg, 0.038 mmol), and K₂CO₃ (105 mg, 0.76 mmol) in 1,4-dioxane (4 mL) and H₂O (1 mL) in a microwave vial was purged with N₂. The vial was capped and subjected to microwave irradiation at 110° C. for 1 h. The crude mixture was purified by column chromatography, eluting with 0 to 12% MeOH in DCM, to afford peak 1 and peak 2. An additional mixture of peak 1 and peak 2 (29 mg) was also isolated and taken on in Step 4.

Peak 1:6-cyclopropyl-4-(2-methyl-2H-pyrazolo[3,4-b]pyridin-5-yl)thieno[2,3-b]pyridine-2-carbaldehyde (28 mg, 0.084 mmol, 33% yield). m/z (ESI): 335.2 (M+H)⁺. ¹H NMR (CHLOROFORM-d, 400 MHz) δ 10.13 (s, 1H), 9.36 (d, J 2.3 Hz, 1H), 8.81 (d, J 2.1 Hz, 1H), 8.24 (s, 1H), 8.03 (s, 1H), 7.37 (s, 1H), 4.31 (s, 3H), 2.41 (tt, J 5.1, 8.4 Hz, 1H), 1.4-1.3 (m, 2H), 1.1-1.0 (m, 2H).

Peak 2:4-cyclopropyl-6-(2-methyl-2H-pyrazolo[3,4-b]pyridin-5-yl)thieno[2,3-b]pyridine-2-carbaldehyde (28 mg, 0.084 mmol,33% yield). m/z(ESI): 335.2 (M+H)⁺. ¹H NMR (CHLOROFORM-d, 400 MHz) δ 10.01 (s, 1H), 8.93 (d, J 2.1 Hz, 1H), 8.27 (d, J 2.3 Hz, 1H), 8.08 (s, 1H), 8.00 (s, 1H), 7.29 (s, 1H), 4.36 (s, 3H), 2.24 (ddd, J 3.3, 4.7, 8.1 Hz, 1H), 1.3-1.2 (m, 2H), 1.2-1.1 (m, 2H).

Step 4: cyclobutyl(4-cyclopropyl-6-(2-methyl-2H-pyrazolo[3,4-b]pyridin-5-yl)thieno[2,3-b]pyridin-2-yl)methanol. To a stirred solution of a mixture of 4-cyclopropyl-6-(2-methyl-2H-pyrazolo[3,4-b]pyridin-5-yl)thieno[2,3-b]pyridine-2-carbaldehyde and 6-cyclopropyl-4-(2-methyl-2H-pyrazolo[3,4-b]pyridin-5-yl)thieno[2,3-b]pyridine-2-carbaldehyde (29.0 mg, 0.087 mmol) in THF (8 mL) at 0° C. under an atmosphere of N₂ was added a solution of cyclobutylmagnesium bromide in THF (0.5 M, 0.87 mL, 0.43 mmol). The resulting mixture was stirred at 0° C. for 0.5 h and quenched by addition of sat. aq. NH₄Cl (1 mL). The volatiles were removed, and the crude mixture was purified by column chromatography, eluting with 0 to 15% MeOH in DCM, to give Compound B7 (3 mg) as the 2^{nd} eluting peak. The first eluting peak was further purified by column chromatography, eluting with 20 to 100% 3:1 EtOAc/EtOH in heptane, to give Compound B8 (7 mg). m/z (ESI): 391.2 (M+H)⁺. ¹H NMR (CHLOROFORM-d, 400 MHz) δ 8.91 (d, J 2.1 Hz, 1H), 8.21 (d, J 2.3 Hz, 1H), 8.03 (s, 1H), 7.17-7.14 (m, 2H), 4.85 (br d, J 7.7 Hz, 1H), 4.33 (s, 3H), 2.8-2.7 (m, 1H), 2.2-1.3 (m, 7H), 1.2-1.1 (m, 2H), 1.1-1.0 (m, 2H).

Alternate conditions in reference to Example 7-1:
(1) To a stirred solution of the product of Step 4 (1 equiv.) in THF (8 mL) and MeOH (2 mL) at rt under an atmosphere of N₂ was added sodium borohydride (13.5 mg, 0.36 mmol). The resulting mixture was stirred at rt for 0.5 h. The volatiles were removed, and the crude residue was purified by column chromatography, eluting with 3 to 20% MeOH in DCM, to give the product.
(2) To a stirred suspension of the alcohol (1 equiv.) in DCM (3 mL) and THF (1 mL) at rt was added NaH (60% dispersion in mineral oil, 2.60 mg, 0.065 mmol) under an atmosphere of N₂. The resulting mixture was stirred at rt for 15 min before iodomethane (4.62 mg, 0.033 mmol) was added. The resulting mixture was stirred at rt for 1.5 h. The crude mixture was purified by column chromatography, eluting with 0 to 16% MeOH in DCM, to give the product.
(3) Step 1: To a stirred suspension of the product of alternate condition (5) (1 equiv.) in 4:1 DCM/THF (0.01 M) at 0° C. was added CsF (1 equiv.) followed by (trifluoromethyl)trimethylsilane (1 equiv.). The resulting mixture was stirred at 0° C. for 5 min before being allowed to warm to rt and stir for 22 h. The mixture was directly purified by column chromatography, eluting with 0 to 15% MeOH in DCM, to afford product.
(3) Continued Step 2: To a stirred solution of the product of alternate condition (3), Step 1 (1 equiv.) in THF (4 mL) at rt was added a solution of tetrabutylammonium fluoride in THF (1.0 M, 1 mL, 1.0 mmol). The resulting mixture was stirred at rt for 0.5 h. The volatiles were removed in vacuo, and the crude residue was purified by column chromatography, eluting with 0 to 20% MeOH in DCM, to give the product.
(4) Step 1: To a stirred solution of 4,6-dichlorothieno[2,3-b]pyridine (2.50 g, 12.3 mmol) and [1,3-bis(2,6-diisopropylphenyl)imidazol-2-ylidene](3-chloropyridyl)palladium(II) dichloride (0.835 g, 1.225 mmol) in THF (10 mL) at 0° C. was added a solution of cyclobutylzinc bromide in THF (0.5 M. 29.4 mL, 14.70 mmol). The reaction was stirred at 0° C. for 30 min. The reaction was diluted with satd. aq. NH₄Cl and extracted with EtOAc. The organic layer was concentrated, and the residue was purified by column chromatography, eluting with 0-40% 3:1 EtOAc/EtOH in heptane, to afford 6-chloro-4-cyclobutylthieno[2,3-b]pyridine (1.45 g, 6.49 mmol, 53% yield). m/z (ESI): 224.1 (M+H)⁺.
(4) Continued Step 2: To a stirred solution of 6-chloro-4-cyclobutylthieno[2,3-b]pyridine (220 mg, 0.983 mmol) in THF (1.5 mL) at −78° C. under N₂ was added a solution of n-butyllithium in THF (2.5 M, 0.472 mL, 1.180 mmol). The resulting mixture was stirred at −78° C. for 30 min before a suspension of formaldehyde (32.5 mg, 1.082 mmol) in THF (1.5 mL) was added via a syringe. The resulting mixture was stirred at −78° C. for 1.5 h before being warmed to rt and stirred for 30 min. The reaction was quenched by addition of cold satd. aq. NH₄Cl, and the mixture was extracted twice with EtOAc. The combined organic layers were dried over Na₂SO₄, filtered and concentrated. The residue was purified by column chromatography, eluting with 0 to 10% MeOH in DCM, to afford crude (6-chloro-4-cyclobutylthieno[2,3-b]pyridin-2-yl)methanol (130 mg), which was taken on directly. m/z (ESI): 254.0 (M+1)+. Additionally, (1-(6-chloro-2-(hydroxymethyl)thieno[2,3-b]pyridin-4-yl)cyclobutyl)methanol (45 mg) was also isolated. m/z (ESI): 284.0 (M+1i.
(5) To a stirred suspension of the product of Step 4 (1 equiv.) in 9:2 DCM/THF (0.015 M) at rt was added Dess-Martin periodinane (1.2 equiv.). The reaction mixture was stirred at rt for 45 ma. The crude mixture was directly purified by column chromatography, eluting with 0 to 14% o MeOH in DCM, to afford the product.

Compounds in Table 7-1 were prepared following the procedure described in Group 7 above using appropriate starting materials. All starting materials are commercially available or are described in the Intermediates section above.

TABLE 7-1

| Ex. # | Chemical Structure & Name | LCMS: (ESI + ve ion) m/z; NMR | Reaction Comments |
|---|---|---|---|
| A2 | 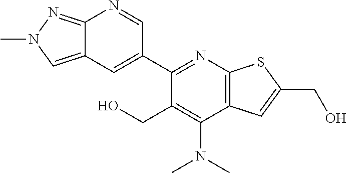 [SMILES: CN(C)c1c(CO)c(-c2cnc3nn(C)cc3c2)nc2sc(CO)cc12] | 370.0; ¹H NMR (METHANOL-d₄, 400 MHz) δ 8.88 (d, J = 2.1 Hz, 1H), 8.55 (d, J = 2.1 Hz, 1H), 8.38 (s, 1H), 7.49 (s, 1H), 4.87 (d, J = 1.0 Hz, 2H), 4.68 (s, 2H), 4.30 (s, 3H), 3.25 (s, 6H) | Compound resulted from a minor product isolated in Step 1; Step 2 and 3: N/A; Alternate condition (1) was used; |
| | (4-(dimethylamino)-6-(2-methyl-2H-pyrazolo | | |
| | [3,4-b]pyridin-5-yl)thieno[2,3-b]pyridine-2,5-diyl)dimethanol | | |
| A3 | 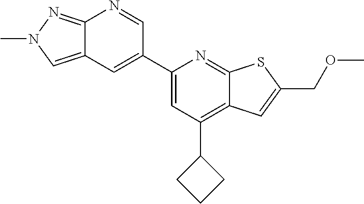 | 365.0; ¹H NMR (400 MHz, CHLOROFORM-d) δ 9.39 (d, J = 2.1 Hz, 1H), 8.73 (d, J = 2.3 Hz, 1H), 7.99 (s, 1H), 7.61 (s, 1H), 7.18 (d, J = 0.8 Hz, 1H), 4.72 (s, 2H), 4.28 (s, 3H), 4.1-3.9 (m, 1H), 3.47 (s, 3H), 2.6-2.5 (m, 2H), 2.37 (dquin, J = 2.3, 9.3 Hz, 2H), 2.3- 2.1 (m, 1H), 2.1-2.0 (m, 1H). | Alternate condition (4) was used instead of Steps 1- 3; Alternate condition (2) was used before Step 4 |
| | 5-(4-cyclobutyl-2-(methoxymethyl)thieno[2,3- | | |
| | b]pyridin-6-yl)-2-methyl-2H-pyrazolo[3,4-b]pyridine | | |
| A4 | 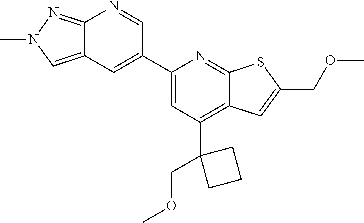 | 409.0; ¹H NMR (400 MHz, CHLOROFORM-d) δ 9.39 (d, J = 2.3 Hz, 1H), 8.75 (d, J = 2.1 Hz, 1H), 7.99 (s, 1H), 7.50 (s, 1H), 7.11 (s, 1H), 4.73 (s, 2H), 4.30 (s, 3H), 3.75 (s, 2H), 3.48 (s, 3H), 3.27 (s, 3H), 2.7-2.5 (m, 4H), 2.3-2.1 (m, 1H), 2.0-1.9 (m, 1H). | Alternate condition (4) was used instead of Steps 1- 3; Alternate condition (2) was used before Step 4 |
| | 5-(2-(methoxymethyl)-4-(1-(methoxymethyl)cyclobutyl) | | |
| | thieno[2,3-b]pyridin-6-yl)-2-methyl-2H-pyrazolo[3,4-b] | | |
| | pyridine | | |
| B1 | 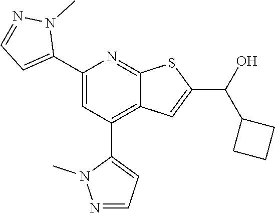 [SMILES: Cn1nccc1-c1cc(-c2ccnn2C)c2cc(C(O)C3CCC3)sc2n1] | 380.2; ¹H NMR (CHLOROFORM-d, 400 MHz) δ 7.65 (d, J = 1.9 Hz, 1H), 7.6-7.5 (m, 2H), 7.05 (s, 1H), 6.64 (d, J = 1.9 Hz, 1H), 6.49 (d, J = 1.7 Hz, 1H), 4.91 (d, J = 7.7 Hz, 1H), 4.33 (s, 3H), 3.86 (s, 3H), 3.50 (s, 1H), 2.8-2.7 (m, 1H), 2.2-2.1 (m, 2H), 2.0-1.9 (m, 4H) | Step 4: N/A |
| | (4,6-bis(1-methyl-1H-pyrazol-5-yl)thieno[2,3-b] | | |
| | pyridin-2-yl)(cyclobutyl)methanol | | |
| B3 | 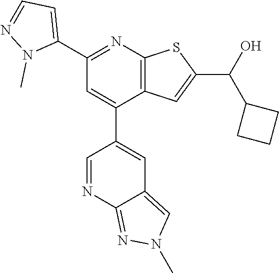 [SMILES: Cn1cc2cc(-c3cc(-c4ccnn4C)nc4sc(C(O)C5CCC5)cc34)cnc2n1] | 431.2; ¹H NMR (CHLOROFORM-d, 400 MHz) δ 8.93 (d, J = 2.1 Hz, 1H), 8.25 (d, J = 2.1 Hz, 1H), 8.05 (s, 1H), 7.59 (s,1H), 7.53 (d, J = 1.9 Hz, 1H), 7.3-7.2 (m, 2H), 4.91 (br d, J = 7.5 Hz, 1H), 4.34 (s, 3H), 4.32 (s, 3H), 2.9-2.7 (m, 1H), 2.47 (br s, 1H), 2.2-1.8 (m, 6H) | |
| | cyclobutyl(4-(2-methyl-2H-pyrazolo[3,4-b] | | |
| | pyridin-5-yl)-6-(1-methyl-1H-pyrazol-5- | | |
| | yl)thieno[2,3-b]pyridin-2-yl)methanol | | |
| B4 | 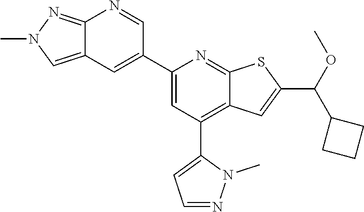 [SMILES: COC(c1cc2c(-c3ccnn3C)cc(-c3cnc4nn(C)cc4c3)nc2s1)C1CCC1] | 445.2; ¹H NMR (CHLOROFORM-d, 400 MHz) δ 9.42 (d, J = 2.1 Hz, 1H), 8.78 (s, 1H), 8.03 (s, 1H), 7.73 (s, 1H), 7.68 (d, J = 1.7 Hz, 1H), 7.06 (s, 1H), 6.54 (d, J = 1.9 Hz, 1H), 4.4-4.3 (m, 4H), 3.91 (s, 3H), 3.38 (s, 3H), 2.8-2.6 (m, 1H), 2.2- 2.0 (m, 2H), 2.0-1.8 (m, 4H) | Alternate condition (2) was used after Step 4 |
| | (cyclobutyl(methoxy)methyl)-4-(1-methyl-1H-pyrazol-5- | | |
| | yl)thieno[2,3-b]pyridin-6-yl)-2-methyl-2H-pyrazolo[3,4- | | |
| | b]pyridine | | |
| B5 | 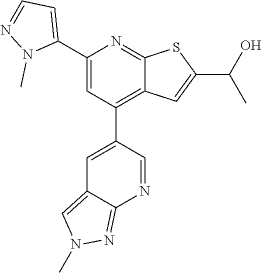 [SMILES: CC(O)c1cc2c(-c3cnc4nn(C)cc4c3)cc(-c3ccnn3C)nc2s1] | 391.0; ¹H NMR (CHLOROFORM-d, 400 MHz) δ 9.54 (d, J = 2.1 Hz, 1H), 8.97 (d, J = 2.1 Hz, 1H), 8.13 (s, 1H), 7.76 (s, 1H), 7.69 (d, J = 1.9 Hz, 1H), 7.07 (d, J = 1.0 Hz, 1H), 6.54 (d, J = 1.9 Hz, 1H), 5.3-5.2 (m, 1H), 4.35 (s, 3H), 3.90 (s, 3H), 2.73 (br s, 1H), 1.70 (d, J = 6.5 Hz, 3H) | |
| | 1-(4-(2-methyl-2H-pyrazolo[3,4-b]pyridin- | | |
| | 5-yl)-6-(1-methyl-1H-pyrazol-5-yl)thieno | | |
| | [2,3-b]pyridin-2-yl)ethanol | | |
| B7 | 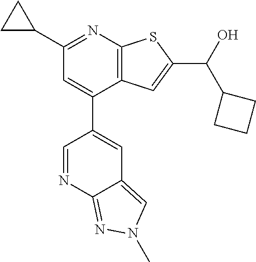 [SMILES: Cn1cc2cc(-c3cc(C4CC4)nc4sc(C(O)C5CCC5)cc34)cnc2n1] | 391.2; ¹H NMR (CHLOROFORM-d, 400 MHz) δ 9.32 (d, J = 2.3 Hz, 1H), 8.70 (d, J = 2.3 Hz, 1H), 7.98 (s, 1H), 7.37 (s, 1H), 7.25 (s, 1H), 4.95 (br d, J = 7.7 Hz, 1H), 4.29 (s, 3H), 2.9-2.7 (m, 1H), 2.4-2.2 (m, 2H), 2.2-1.9 (m, 6H), 1.3-1.2 (m, 2H), 1.0-1.0 (m, 2H) | |
| | cyclobutyl(6-cyclopropyl-4-(2-methyl-2H- | | |
| | pyrazolo[3,4-b]pyridin-5-yl)thieno[2,3-b] | | |
| | pyridin-2-yl)methanol | | |
| B10 | 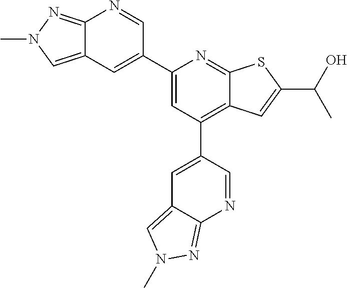 [SMILES: CC(O)c1cc2c(-c3cnc4nn(C)cc4c3)cc(-c3cnc4nn(C)cc4c3)nc2s1] | 442.0; ¹H NMR (CHLOROFORM-d, 400 MHz) δ 9.36 (d, J = 2.1 Hz, 1H), 8.87 (d, J = 1.9 Hz, 1H), 8.71 (d, J = 2.1 Hz, 1H), 8.26 (d, J = 2.1 Hz, 1H), 8.07 (s, 1H), 7.99 (s, 1H), 7.71 (s, 1H), 7.20 (s, 1H), 5.22 (q, J = 6.1 Hz, 1H), 4.33 (s, 3H), 4.28 (s, 3H), 3.17 (br s, 1H), 1.67 (br d, J = 6.3 Hz, 3H) | Step 4: N/A |
| | 1-(4,6-bis(2-methyl-2H-pyrazolo[3,4-b]pyridin-5- | | |
| | yl)thieno[2,3-b]pyridin-2-yl)ethanol | | |
| B11 | 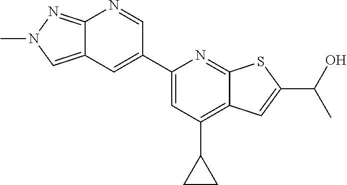 [SMILES: CC(O)c1cc2c(C3CC3)cc(-c3cnc4nn(C)cc4c3)nc2s1] | 351.2; ¹H NMR (CHLOROFORM-d, 400 MHz) δ 9.28 (d, J = 2.1 Hz, 1H), 8.69 (d, J = 2.3 Hz, 1H), 7.98 (s, 1H), 7.36 (s, 1H), 7.23 (s, 1H), 5.25 (br d, J = 5.9 Hz, 1H), 4.28 (s, 3H), 2.61 (br s, 1H), 2.28 (tt, J = 5.2, 8.4 Hz, 1H), 1.72 (d, J = 6.5 Hz, 3H), 1.3-1.2 (m, 2H), 1.0-0.9 (m, 2H) | |
| | 1-(4-cyclopropyl-6-(2-methyl-2H-pyrazolo[3,4-b]pyridin- | | |
| | 5-yl)thieno[2,3-b]pyridin-2-yl)ethanol | | |
| B11S | 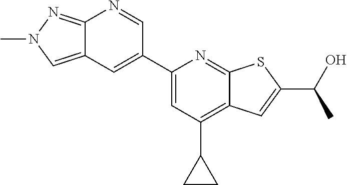 [SMILES: C[C@H](O)c1cc2c(C3CC3)cc(-c3cnc4nn(C)cc4c3)nc2s1] | 351.2; ¹H NMR (DMSO-d₆, 500 MHz) δ 9.36 (d, J = 2.3 Hz, 1H), 8.89 (d, J = 2.3 Hz, 1H), 8.50 (s, 1H), 7.50 (d, J = 1.0 Hz, 1H), 7.47 (s, 1H), 5.1-5.0 (m, 1H), 4.23 (s, 3H), 1.54 (d, J = 6.5 Hz, 3H), 1.2- 1.1 (m, 2H), 1.1-1.1 (m, 2H) | After Step 4, the product was purified by SFC; Peak 2/SFC: (S,S) Whelk-O1, 21 x 250 mm 5μm; mobile phase: 55% CO₂: 45% MeOH(0.2% DEA, v/v) using a flow rate of 80 |
| | (1S)-1-(4-cyclopropyl-6-(2-methyl-2H-pyrazolo[3,4- | | mL/min |
| | b]pyridin-5-yl)thieno[2,3-b]pyridin-2-yl)ethanol | | |
| B11R | 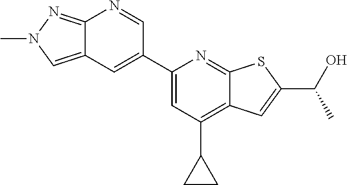 [SMILES: C[C@@H](O)c1cc2c(C3CC3)cc(-c3cnc4nn(C)cc4c3)nc2s1] | 351.2; ¹H NMR (CHLOROFORM-d with drops of MeOH-d4, 400 MHz) δ 9.20 (s, 1H), 8.62 (d, J = 1.3 Hz, 1H), 8.00 (s, 1H), 7.33 (s, 1H), 7.18 (s, 1H), 5.13 (q, J = 6.4 Hz, 1H), 4.22 (s, 3H), 2.3-2.2 (m, 1H), 1.62 (d, J = 6.5 Hz, 3H), 1.2-1.1 (m, 2H), 1.0-0.9 (m, 2H) | After Step 4, the product was purified by SFC; Peak 1/SFC: (S,S) Whelk-O1, 21 x 250 mm 5μm; mobile phase: 55% CO₂: 45% MeOH(0.2% DEA, v/v) using a flow rate of 80 |
| | (1R)-1-(4-cyclopropyl-6-(2-methyl-2H-pyrazolo[3,4- | | mL/min |
| | b]pyridin-5-yl)thieno[2,3-b]pyridin-2-yl)ethanol | | |
| B12 | 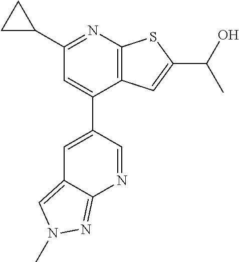 [SMILES: CC(O)c1cc2c(-c3cnc4nn(C)cc4c3)cc(C3CC3)nc2s1] | 351.2; ¹H NMR (CHLOROFORM-d, 400 MHz) δ 8.90 (d, J = 2.3 Hz, 1H), 8.21 (d, J = 2.1 Hz, 1H), 8.02 (s, 1H), 7.16 (s, 1H), 7.15 (2, 1H), 5.2-5.1 (m, 1H), 4.33 (s, 3H), 2.2- 2.1 (m, 2H), 1.64 (d, J = 6.3 Hz, 3H), 1.17 (dd, J = 2.3, 4.8 Hz, 2H), 1.08 (td, J = 3.0, 8.0 Hz, 2H) | |
| | 1-(6-cyclopropyl-4-(2-methyl-2H- | | |
| | pyrazolo[3,4-b]pyridin-5- | | |
| | yl)thieno[2,3-b]pyridin-2-yl)ethanol | | |
| B13 | 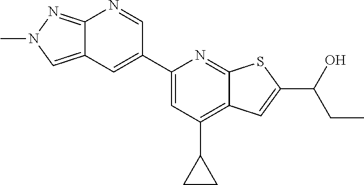 [SMILES: CCC(O)c1cc2c(C3CC3)cc(-c3cnc4nn(C)cc4c3)nc2s1] | 365.2; ¹H NMR (CHLOROFORM-d, 400 MHz) δ 9.32 (d, J = 2.3 Hz, 1H), 8.71 (d, J = 2.3 Hz, 1H), 7.99 (s, 1H), 7.39 (s, 1H), 7.26 (s, 1H), 4.98 (br t, J = 6.3 Hz, 1H), 4.29 (s, 3H), 2.4-2.3 (m, 2H), 2.00 (quin, J = 7.2 Hz, 2H), 1.3-1.2 (m, 2H), 1.07 (t, J = 7.4 Hz, 3H), 1.0-1.0 (m, 2H) | |
| | 1-(4-cyclopropyl-6-(2-methyl-2H-pyrazolo[3,4-b]pyridin-5- | | |
| | yl)thieno[2,3-b]pyridin-2-yl)-1-propanol | | |
| B13R | 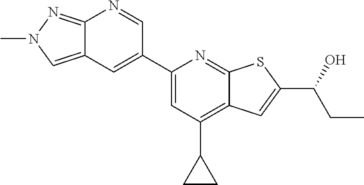 [SMILES: CC[C@@H](O)c1cc2c(C3CC3)cc(-c3cnc4nn(C)cc4c3)nc2s1] | 365.2; ¹H NMR(CHLOROFORM-d with drops of MeOH-d₄, 400 MHz) δ 9.18 (s, 1H), 8.6-8.6 (m, 1H), 8.01 (s, 1H), 7.32 (d, J = 0.6 Hz, 1H), 7.16 (d, J = 1.0 Hz, 1H), 4.84 (t, 1H, J = 6.6 Hz), 4.20 (s, 3H), 2.3-2.2 (m, 1H), 1.89 (quin, J = 7.1 Hz, 2H), 1.2-1.1 (m, 2H), 1.0-0.9 (m, 5H) | After Step 4, the product was purified by SFC; Peak 1/SFC: Lux Cellulose-2, 2 x 25 cm 5 μm; mobile phase: 55% CO₂: 45% MeOH (0.2% DEA, v/v) using a flow rate of 80 mL/min |
| | (1R)-1-(4-cyclopropyl-6-(2-methyl-2H-pyrazolo[3,4- | | |
| | b]pyridin-5-yl)thieno[2,3-b]pyridin-2-yl)-1-propanol | | |
| B13S | 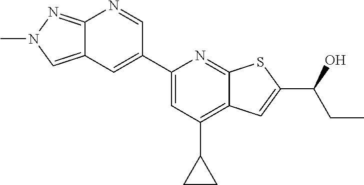 [SMILES: CC[C@H](O)c1cc2c(C3CC3)cc(-c3cnc4nn(C)cc4c3)nc2s1] | 365.2; ¹H NMR (CHLOROFORM-d with drops of MeOH-d₄, 400 MHz) δ 9.18 (s, 1H), 8.6-8.6 (m, 1H), 8.01 (s, 1H), 7.32 (s, 1H), 7.17 (d, J = 0.8 Hz, 1H), 4.85 (t, J = 6.2 Hz, 1H), 4.21 (s, 3H), 2.3-2.2 (m, 1H), 2.0- 1.8 (m, 2H), 1.2-1.1 (m, 2H), 1.0- 0.9 (m, 5H) | After Step 4, the product was purified by SFC; Peak 2/SFC: Lux Cellulose-2, 2 x 25 cm 5 μm; mobile phase: 55% CO₂: 45% MeOH (0.2% DEA, v/v) using a flow rate of 80 mL/min |
| | (1S)-1-(4-cyclopropyl-6-(2-methyl-2H-pyrazolo[3,4- | | |
| | b]pyridin-5-yl)thieno[2,3-b]pyridin-2-yl)-1-propanol | | |
| C23 | 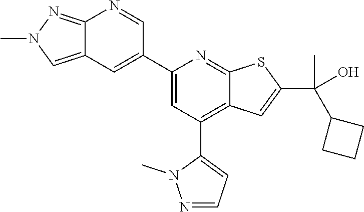 [SMILES: Cn1cc2cc(-c3cc(-c4ccnn4C)c4cc(C(C)(O)C5CCC5)sc4n3)cnc2n1] | 445.2; ¹H NMR (CHLOROFORM-d, 400 MHz) δ 9.42 (d, J = 2.3 Hz, 1H), 8.77 (d, J = 2.3 Hz, 1H), 8.02 (s, 1H), 7.71 (s, 1H), 7.67 (d, J = 1.9 Hz, 1H), 6.98 (s, 1H), 6.52 (d, J = 1.9 Hz, 1H), 4.31 (s, 3H), 3.89 (s, 3H), 2.9-2.7 (m, 1H), 2.2-2.0 (m, 4H), 1.9-1.8 (m, 3H), 1.58 (s, 3H) | Alternate condition (5) was used followed by an additional Grignard addition analogous to Step 2 |
| | 1-cyclobutyl-1-(6-(2-methyl-2H-pyrazolo[3,4-b]pyridin-5- | | |
| | yl)-4-(1-methyl-1H-pyrazol-5-yl)thieno[2,3-b]pyridin-2-yl) | | |
| | ethanol | | |
| C24 | 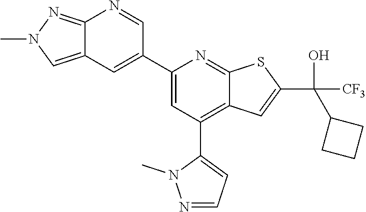 [SMILES: Cn1cc2cc(-c3cc(-c4ccnn4C)c4cc(C(O)(C5CCC5)C(F)(F)F)sc4n3)cnc2n1] | 499.2; ¹H NMR (CHLOROFORM-d, 400 MHz) δ 9.43 (d, J = 2.3 Hz, 1H), 8.78 (d, J = 2.3 Hz, 1H), 8.04 (s, 1H), 7.76 (s, 1H), 7.69 (d, J = 1.9 Hz, 1H), 7.22 (s, 1H), 6.54 (d, J = 1.9 Hz, 1H), 4.31 (s, 3H), 3.90 (s, 3H), 3.3-3.1 (m, 1H), 2.98 (br s, 1H), 2.3-2.2 (m, 1H), 2.1-1.9 (m, 3H), 1.8-1.7 (m, 2H) ¹⁹F NMR (CHLOROFORM-d, 376 MHz) δ −78.24 (s, 3F) | Alternate condition (3) was used |
| | 1-cyclobutyl-2,2,2-trifluoro-1-(6-(2-methyl-2H-pyrazolo | | |
| | [3,4-b]pyridin-5-yl)-4-(1-methyl-1H-pyrazol-5-yl)thieno | | |
| | [2,3-b]pyridin-2-yl)ethanol | | |

### Group 8

Example 8-1: (R)-1-(6-(2-methyl-2H-pyrazolo[3,4-b]pyridin-5-yl)-4-(1-methyl-1H-pyrazol-5-yl)thieno[2,3-b]pyridin-2-yl)ethanol B6R; (S)-1-(6-(2-methyl-2H-pyrazolo[3,4-b]pyridin-5-yl)-4-(1-methyl-1H-pyrazol-5-yl)thieno[2,3-b]pyridin-2-yl)ethanol B6S

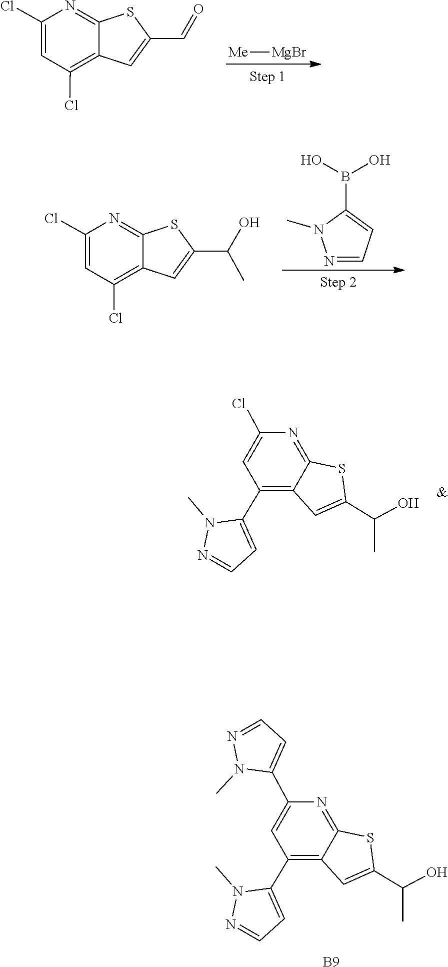

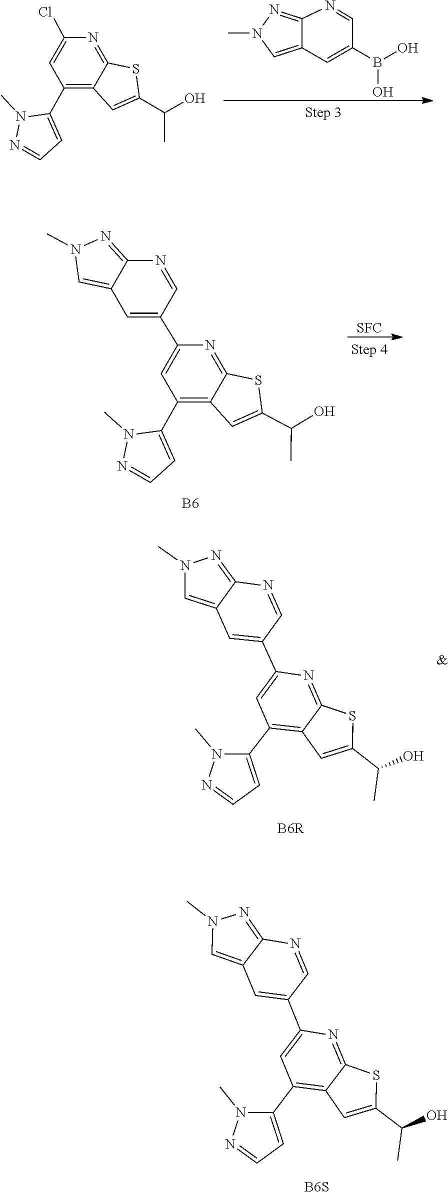

Step 1: 1-(4,6-dichlorothieno[2,3-b]pyridin-2-yl)ethan-1-ol. To a stirred solution of 4,6-dichlorothieno[2,3-b]pyridine-2-carbaldehyde (1.95 g, 8.40 mmol) in THF (40 mL mL) and DCM (40 mL) at 0° C. under N₂ was added a solution of methylmagnesium bromide in THF (3.0 M, 5.6 mL, 16.8 mmol) via syringe. The resulting mixture was stirred at 0° C. for 20 min and then quenched by addition of 0° C. sat. aq. NH₄Cl (4 mL). The mixture was concentrated, and the resulting residue was purified by column chromatography, eluting with 0 to 20% MeOH in DCM, to give 1-(4,6-dichlorothieno[2,3-b]pyridin-2-yl)ethan-1-ol (1.78 g, 7.17 mmol, 85% yield). m/z (ESI): 248.0 (M+H)⁺. ¹H NMR (400 MHz, CHLOROFORM-d) δ 7.35 (s, 1H), 7.24 (d, J=1.0 Hz, 1H), 5.21 (br d, J=6.4 Hz, 1H), 2.26 (br d, J=1.5 Hz, 1H), 1.68 (d, J=6.4 Hz, 3H).

Step 2: 1-(6-chloro-4-(1-methyl-1H-pyrazol-5-yl)thieno[2,3-b]pyridin-2-yl)ethan-1-ol. A mixture of 1-(4,6-dichlorothieno[2,3-b]pyridin-2-yl)ethan-1-ol (577 mg, 2.33 mmol), (1-methyl-1H-pyrazol-5-yl)boranediol (302 mg, 2.40 mmol), cataCXium A Pd G3 (169 mg, 0.23 mmol), and K₃PO₄ monohydrate (1.61 g, 6.98 mmol) in THF (12.0 mL) and H₂O (2.0 mL) in a microwave vial was purged with N₂ for 5 min, capped and subjected to microwave irradiation at 68° C. for 75 min. The volatiles were removed, and the crude residue was purified by column chromatography, eluting with 0 to 20% MeOH in DCM, to give 1-(6-chloro-4-(1-methyl-1H-pyrazol-5-yl)thieno[2,3-b]pyridin-2-yl)ethan-1-ol (425 mg). m/z (ESI): 294.0 (M+H)⁺. Additionally, 25.5 mg of Compound B9 (1-(4,6-bis(1-methyl-1H-pyrazol-5-yl)thieno[2,3-b]pyridin-2-yl)ethanol) was isolated. m/z (ESI): 340.0 (M+H)⁺. ¹H NMR (CHLOROFORM-d, 400 MHz) δ 7.64 (d, J=1.9 Hz, 1H), 7.55-7.50 (m, 2H), 7.06 (s, 1H), 6.64 (d, J=2.1 Hz, 1H), 6.49 (d, J=1.9 Hz, 1H), 5.22 (quin, J=5.8 Hz, 1H), 4.32 (s, 3H), 3.86 (s, 3H), 2.59 (d, J=4.6 Hz, 1H), 1.68 (d, J=6.3 Hz, 3H).

Step 3: 1-(4-(1-methyl-1H-pyrazol-5-yl)-6-(2-methyl-2H-pyrazolo[3,4-b]pyridin-5-yl)thieno[2,3-b]pyridin-2-yl)ethan-1-ol. This reaction was set up in duplicate. A mixture of 1-(6-chloro-4-(1-methyl-1H-pyrazol-5-yl)thieno[2,3-b]pyridin-2-yl)ethan-1-ol (625 mg, 2.13 mmol), (2-methyl-2H-pyrazolo[3,4-b]pyridin-5-yl)boronic acid (602 mg, 3.40 mmol), Pd(dppf)Cl₂ (234 mg, 0.32 mmol), and K₂CO₃ (882 mg, 6.38 mmol) in 1,4-dioxane (12 mL) and H₂O (1.5 mL) in a microwave vial was purged with N₂ for 5 min. The vial was capped and subjected to microwave irradiation at 105° C. for 1.5 h. The crude mixture was concentrated, combined with the duplicate reaction, and purified directly by column chromatography, eluting with 0 to 20% MeOH in DCM. The resulting product was triturated with EtOAc/heptane to provide Compound B6 (718 mg). m/z (ESI): 391.2 (M+H)⁺. ¹H NMR (CHLOROFORM-d, 400 MHz) δ 9.03 (d, J 2.1 Hz, 1H), 8.36 (d, J 2.1 Hz, 1H), 8.12 (s, 1H), 7.63 (s, 1H), 7.60 (d, J 2.1 Hz, 1H), 7.29 (d, J 0.8 Hz, 1H), 6.71 (d, J 2.1 Hz, 1H), 5.3-5.2 (m, 1H), 4.37 (s, 3H), 4.36 (s, 3H), 2.95 (br s, 1H), 1.69 (d, J 6.5 Hz, 3H).

Step 4: SFC Separation. The sample was purified via SFC using a Chiralcel OJ, 21×250 mm, 5 μm column with a mobile phase of 40% EtOH with 0.2% DEA using a flowrate of 100 mL/min to generate peak 1 and peak 2. Peak 1: B6R. Peak 1 was further purified by column chromatography, eluting with 2 to 20% MeOH in DCM. The resulting product was then purified again by preparative reverse-phase HPLC, eluting with a gradient of 15 to 95% ACN in H₂O (0.1% TFA). The resulting TFA salt was dissolved in MeOH/DCM, and to this solution was added aq. NH₄OH (0.8 mL). The resulting mixture was poured through a bed of Na₂SO₄ and directly purified by column chromatography, eluting with 1 to 20% MeOH in DCM, to afford Compound B6R (260 mg). m/z (ESI): 391.1 (M+H)⁺. ¹H NMR (DMSO-d₆, 500 MHz) δ 9.44 (s, 1H), 9.01 (s, 1H), 8.55 (s, 1H), 8.16 (d, J 1.0 Hz, 1H), 7.72-7.60 (m, 1H), 7.11 (s, 1H), 6.71-6.61 (m, 1H), 5.86 (d, J 4.4 Hz, 1H), 5.21-5.00 (m, 1H), 4.24 (s, 3H), 3.90 (d, J 1.0 Hz, 3H), 1.49 (d, J 6.4 Hz, 3H).

Peak 2: B6S. Peak 2 was further purified by column chromatography, eluting with 2 to 20% MeOH in DCM, to afford Compound B6S (310 mg). m/z (ESI): 391.1 (M+H)⁺. ¹H NMR (DMSO-d₆, 500 MHz) δ 9.45 (d, J 1.8 Hz, 1H), 9.01 (d, J 1.9 Hz, 1H), 8.55 (s, 1H), 8.16 (s, 1H), 7.66 (d, J 1.6 Hz, 1H), 7.12 (s, 1H), 6.66 (d, J 1.6 Hz, 1H), 5.87 (d, J 4.5 Hz, 1H), 5.08 (quin, J 5.7 Hz, 1H), 4.24 (s, 3H), 3.90 (s, 3H), 1.49 (d, J 6.5 Hz, 3H).

### Group 9

Example 9-1: (3-amino-6-(5-pyrimidinyl)thieno[2,3-b]pyridin-2-yl)(3,3-difluorocyclobutyl)methanol B71

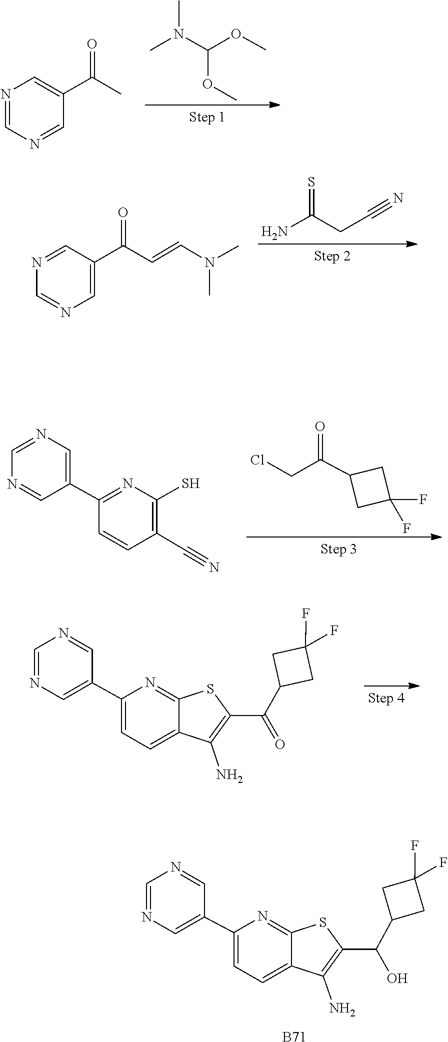

Step 1: (E)-3-(dimethylamino)-1-(pyrimidin-5-yl)prop-2-en-1-one. To a solution of 1-(pyrimidin-2-yl)ethan-1-one (5.00 g, 40.9 mmol) in IPA was added 1,1-dimethoxy-N,N-dimethylmethanamine (7.32 g, 61.4 mmol). The mixture was stirred at 100° C. for 12 h. MTBE was added to the reaction solution to precipitate solids. The solid was filtered to provide (E)-3-(dimethylamino)-1-(pyrimidin-5-yl)prop-2-en-1-one (3.50 g, 19.75 mmol, 48% yield). ¹H NMR (400 MHz, DMSO-d₆) δ 9.27 (s, 1H), 9.22 (s, 2H), 7.82 (d, J 12.1 Hz, 1H), 5.91 (br d, J 12.1 Hz, 1H), 3.19 (s, 3H), 2.97 (s, 3H).

Step 2: 2-mercapto-6-(pyrimidin-5-yl)nicotinonitrile. To a solution of 2-cyanoethanethioamide (0.848 g, 8.46 mmol) and DABCO (0.317 g, 2.82 mmol) in EtOH (10 mL) was added (E)-3-(dimethylamino)-1-(pyrimidin-5-yl)prop-2-en-1-one (1.00 g, 5.64 mmol). The mixture was stirred at 80° C. for 12 h. MTBE (100 mL) was added to the reaction mixture. The product was collected by filtration, and the filter cake was dried under reduced pressure to obtain 2-mercapto-6-(pyrimidin-5-yl)nicotinonitrile (900 mg, 4.20 mmol, 74% yield). ¹H NMR (400 MHz, DMSO-d₆) δ 9.31 (s, 2H), 9.20 (s, 1H), 7.57 (d, J 7.8 Hz, 1H), 7.19 (d, J 7.9 Hz, 1H).

Step 3: (3-amino-6-(pyrimidin-5-yl)thieno[2,3-b]pyridin-2-yl)(3,3-difluorocyclobutyl)methanone. To a solution of 2-mercapto-6-(pyrimidin-5-yl)nicotinonitrile (330 mg, 1.54 mmol) in DMF (3 mL) was added KOH (108 mg, 1.93 mmol). The solution was stirred at 20° C. for 12 h, then 2-chloro-1-(3,3-difluorocyclobutyl)ethan-1-one (260 mg, 1.54 mmol) was added. The mixture was stirred at 20° C. for 2 h. The reaction mixture was poured into H₂O (20 mL) and extracted with EtOAc (3×40 mL). The combined organic layers were washed with brine (2×30 mL), dried over Na₂SO₄, filtered and concentrated. The residue was purified by column chromatography, eluting with 100% EtOAc, to give (3-amino-6-(pyrimidin-5-yl)thieno[2,3-b]pyridin-2-yl)(3,3-difluorocyclobutyl)methanone (180 mg, 0.52 mmol, 40.5% yield). ¹H NMR (400 MHz, DMSO-d₆) δ 9.54 (s, 2H), 9.31 (s, 1H), 8.78 (d, J 8.5 Hz, 1H), 8.26 (d, J 8.5 Hz, 1H), 8.19 (s, 2H), 3.51 (dt, J 3.1, 8.3 Hz, 1H), 2.92 (br dd, J 8.4, 15.6 Hz, 4H).

Step 4: (3-amino-6-(5-pyrimidinyl)thieno[2,3-b]pyridin-2-yl)(3,3-difluorocyclobutyl)methanol. To a mixture of (3-amino-6-(pyrimidin-5-yl)thieno[2,3-b]pyridin-2-yl)(3,3-difluorocyclobutyl)methanone (180 mg, 0.52 mmol) in THF (2 mL) was added NaBH₄ (59.0 mg, 1.56 mmol) slowly at 0° C. under an atmosphere of N₂. The mixture was stirred at 0° C. for 14 h. The reaction mixture was quenched by addition of H₂O and extracted with EtOAc (3×40 mL). The combined organic layers were washed with brine (2×30 mL), dried over Na₂SO₄, filtered and concentrated under reduced pressure. The residue was purified by column chromatography, eluting with a gradient of 0%-100% EtOAc in pet. ether, to give the crude product. The product was purified again by prep-HPLC, eluting with a mobile phase of 5%-95% ACN in (0.1% HCOOH) H₂O, to afford Compound B71 (4.1 mg, 0.012 mmol, 2.3% yield). m/z (ESI): 349.3 (M+H)⁺. ¹H NMR (400 MHz, DMSO-d₆) δ 9.49 (s, 2H), 9.24 (s, 1H), 8.33 (d, J 8.4 Hz, 1H), 8.12 (d, J 8.4 Hz, 1H), 5.90 (J 4.4 Hz, 1H), 5.44 (s, 2H), 5.11 (t, J 4.2 Hz, 1H), 2.70-2.55 (m, 5H).

### Group 10

Example 10-1:3,3-difluoro-1-(6-(3-methyl-3H-imidazo[4,5-b]pyridin-6-yl)thieno[2,3-b]pyridin-2-yl)cyclobutanol C33

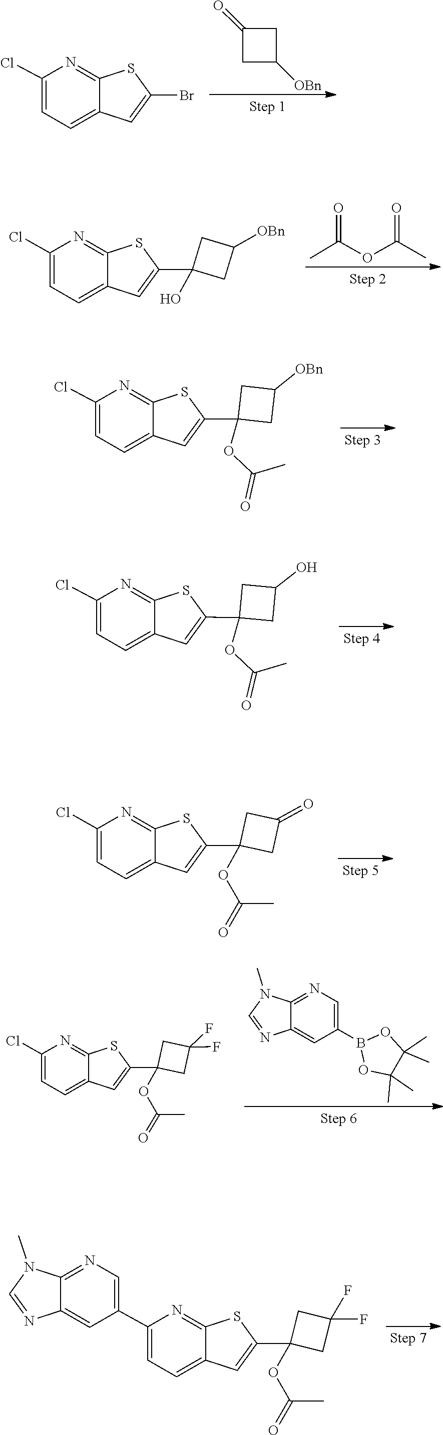

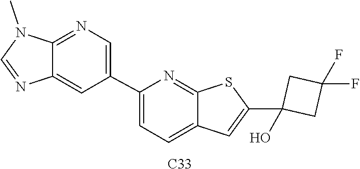 [SMILES: Cn1cnc2cc(-c3ccc4cc(C5(O)CC(F)(F)C5)sc4n3)cnc21 ; "C33"]

Step 1: 3-(benzyloxy)-1-(6-chlorothieno[2,3-b]pyridin-2-yl)cyclobutan-1-ol. To a solution of 2-bromo-6-chlorothieno[2,3-b]pyridine (5 g, 20.12 mmol) and 3-(benzyloxy)cyclobutan-1-one (8.86 g, 50.3 mmol) in THF (100 mL) at −78° C. was added a solution of n-butyllithium in hexanes (2.5 M, 20.12 mL, 50.3 mmol) dropwise, and the reaction mass was stirred at −78° C. for 30 min. Then, the mixture was quenched by addition of sat. aq. NH₄C₁, extracted with EtOAc (200 mL×3), washed with brine (100 mL), dried over Na₂SO₄, and concentrated under reduced pressure. The crude material was purified by column chromatography, eluting with a gradient of 30% EtOAc in pet. ether, to obtain 3-(benzyloxy)-1-(6-chlorothieno[2,3-b]pyridin-2-yl)cyclobutan-1-ol (3 g, 8.67 mmol, 43% yield). m/z (ESI): 346.0 (M+H)⁺. ¹H NMR (400 MHz, DMSO-d₆) δ 8.18 (d, J=8.4 Hz, 1H), 7.47 (d, J=8.4 Hz, 1H), 7.28-7.45 (m, 6H), 6.46 (s, 1H), 4.43-4.36 (m, 2H), 4.05-3.95 (m, 1H), 2.86-2.82 (m, 2H), 2.43-2.38 (m, 2H).

Step 2: 3-(benzyloxy)-1-(6-chlorothieno[2,3-b]pyridin-2-yl)cyclobutyl acetate. To a solution of 3-(benzyloxy)-1-(6-chlorothieno[2,3-b]pyridin-2-yl)cyclobutan-1-ol (3 g, 8.67 mmol) in DCM (90 mL) at 0° C. was added acetic anhydride (4.09 mL, 43.4 mmol), Et₃N (2.418 mL, 17.35 mmol) and N,N-dimethylpyridin-4-amine (0.212 g, 1.74 mmol). The reaction mass was slowly warmed to rt and stirred for 2 h, and then the mixture was concentrated under reduced pressure. The residue was diluted with H₂O (200 mL), extracted with DCM (200 mL×3), and concentrated under reduced pressure to give a crude material. The crude material was purified by chromatography, eluting with a gradient of 0% to 8% EtOAc in hexane to provide 3-(benzyloxy)-1-(6-chlorothieno[2,3-b]pyridin-2-yl)cyclobutyl acetate (2.7 g, 6.96 mmol, 80% yield). m/z (ESI): 388.0 (M+H)⁺. ¹H NMR (400 MHz, DMSO-d₆) δ 8.24 (d, J=8.4 Hz, 1H), 7.54-7.52 (m, 2H), 7.35-7.29 (m, 5H), 4.44 (s, 2H), 4.06-3.99 (m, 1H), 3.32-3.09 (m, 2H), 2.58-2.53 (m, 2H), 2.05 (s, 3H).

Step 3: 1-(6-chlorothieno[2,3-b]pyridin-2-yl)-3-hydroxycyclobutyl acetate. To a solution of 3-(benzyloxy)-1-(6-chlorothieno[2,3-b]pyridin-2-yl)cyclobutyl acetate (2.6 g, 6.70 mmol) in DCM (104 mL) at 0° C. was added TiCl₄ (20% w/w in DCM, 16 mL, 33.5 mmol). The reaction mass was warmed to rt and stirred for 4 h. Then, the mixture was quenched by addition of sat. aq. NaHCO₃, extracted with EtOAc, filtered, and washed with H₂O. The solution was dried over Na₂SO₄, filtered, and concentrated under reduced pressure to afford a crude material. The crude material was purified by column chromatography, eluting using a mobile phase of 50% EtOAc in pet. ether, to obtain 1-(6-chlorothieno[2,3-b]pyridin-2-yl)-3-hydroxycyclobutyl acetate (0.8 g, 2.69 mmol, 40% yield). m/z (ESI): 297.9 (M+H)⁺. ¹H NMR (400 MHz, DMSO-d₆) δ 8.24 (d, J=8.4 Hz, 1H), 7.55-7.51 (m, 2H), 5.44 (d, J=6.4 Hz, 1H), 4.04 (p, J=7.1 Hz, 1H), 3.07 (m, 2H), 2.48-2.41 (m, 2H), 2.03 (s, 3H).

Step 4: 1-(6-chlorothieno[2,3-b]pyridin-2-yl)-3-oxocyclobutyl acetate. To a solution of 1-(6-chlorothieno[2,3-b]pyridin-2-yl)-3-hydroxycyclobutyl acetate (0.9 g, 3.02 mmol) in EtOAc (36.0 mL) was added IBX (2.54 g, 9.07 mmol) at rt. The reaction was stirred at 65° C. for 16 h. Then, the reaction was diluted with EtOAc, filtered, and washed with EtOAc. The filtrate was concentrated under reduced pressure to afford crude 1-(6-chlorothieno[2,3-b]pyridin-2-yl)-3-oxocyclobutyl acetate (0.9 g, 2.92 mmol, 97% yield), which was used directly for the next step. m/z (ESI): 296.0 (M+H)⁺. ¹H NMR (400 MHz, DMSO-d₆) δ 8.27 (d, J=8.4 Hz, 1H), 7.65 (s, 1H), 7.56 (d, J=8.4 Hz, 1H), 3.87 (s, 4H), 2.11 (s, 3H).

Step 5: 1-(6-chlorothieno[2,3-b]pyridin-2-yl)-3,3-difluorocyclobutyl acetate. To a solution of 1-(6-chlorothieno[2,3-b]pyridin-2-yl)-3-oxocyclobutyl acetate (0.8 g, 2.71 mmol) in DCM (32.0 mL) was added a solution of DAST (1.07 mL, 8.12 mmol) in DCM (2 mL) at 0° C. Then, the reaction was warmed to rt and stirred for 20 h. After, the reaction was quenched by addition of sat. aq. NaHCO₃, extracted with DCM (200 mL×3), washed with brine (100 mL), dried over Na₂SO₄, and concentrated under reduced pressure to afford a crude material. The crude material was purified by column chromatography, eluting using a mobile phase of 30% EtOAc in pet. ether, to obtain 1-(6-chlorothieno[2,3-b]pyridin-2-yl)-3,3-difluorocyclobutyl acetate (0.4 g, 1.26 mmol, 47% yield). m/z (ESI): 318.0 (M+H)⁺. ¹H NMR (400 MHz, DMSO-d₆) δ 8.29 (d, J=8.4 Hz, 1H), 7.65 (s, 1H), 7.56 (d, J=8.4 Hz, 1H), 3.59-3.38 (m, 4H), 2.08 (s, 3H).

Step 6: 3,3-difluoro-1-(6-(3-methyl-3H-imidazo[4,5-b]pyridin-6-yl)thieno[2,3-b]pyridin-2-yl)cyclobutyl acetate. A solution of 1-(6-chlorothieno[2,3-b]pyridin-2-yl)-3,3-difluorocyclobutyl acetate (0.1 g, 0.32 mmol), 3-methyl-6-(4,4,5,5-tetramethyl-1,3,2-dioxaborolan-2-yl)-3H-imidazo[4,5-b]pyridine (0.122 g, 0.47 mmol) and K₂CO₃ (0.130 g, 0.94 mmol) in 1,4-dioxane (3.60 mL) and H₂O (0.40 mL) was stirred and purged with N₂ gas for 5 min. After purging, Pd(dppf)Cl₂ (0.023 g, 0.031 mmol) was added, and the reaction was stirred at 110° C. for 2 h. Then, the reaction mixture was diluted with EtOAc, filtered, and washed with EtOAc (10 mL). The filtrate was concentrated under reduced pressure to afford crude 3,3-difluoro-1-(6-(3-methyl-3H-imidazo[4,5-b]pyridin-6-yl)thieno[2,3-b]pyridin-2-yl)cyclobutyl acetate (0.15 g, 0.29 mmol, 93% yield), which was used in next step without further purification. m z (ESI): 415.2 (M+H)⁺.

Step 7: 3,3-difluoro-1-(6-(3-methyl-3H-imidazo[4,5-b]pyridin-6-yl)thieno[2,3-b]pyridin-2-yl)cyclobutanol. To a solution of 3,3-difluoro-1-(6-(3-methyl-3H-imidazo[4,5-b]pyridin-6-yl)thieno[2,3-b]pyridin-2-yl)cyclobutyl acetate (0.15 g, 0.36 mmol) in MeOH (4.50 mL) and H₂O (0.45 mL) was added K₂CO₃ (0.150 g, 1.09 mmol) at rt, and the reaction mixture was stirred for 1 h. After, the solution was concentrated under reduced pressure, diluted with EtOAc, filtered, and washed with EtOAc (20 mL). The filtrate was concentrated under reduced pressure to afford a crude material. The crude material was purified by prep-HPLC, eluting with a linear gradient of 5%-95% (0.1% NH₃) H₂O in ACN with a flow rate of 15 mL/min, to provide Compound C33 (0.078 g, 0.210 mmol, 58% yield). m/z (ESI): 373.2 (M+H)⁺. ¹H NMR (400 MHz, DMSO-d₆) δ 9.20 (d, J=2.0 Hz, 1H), 8.77 (d, J=2.0 Hz, 1H), 8.51 (s, 1H), 8.30 (d, J=8.4 Hz, 1H), 8.15 (d, J=8.4 Hz, 1H), 7.46 (s, 1H), 6.85 (s, 1H), 3.91 (s, 3H), 3.24 (td, J =11.6, 9.5, 3.8 Hz, 2H), 3.06 (td, J=14.2, 12.0 Hz, 2H).

### Alternate Conditions:

(1) In Step 6, SPhos Pd G3 was used as the catalyst instead of Pd(dppf)Cl₂

Compounds in Table 10-1 were prepared following the procedure described in Group 10, using appropriate starting materials (e.g., aldehyde/ketones and boronic esters). All starting materials are commercially available or are described in the Intermediates section above.

TABLE 10-1

| | | | Reaction |
|---|---|---|---|
| Ex. # | Chemical Structure & Name | LCMS: (ESI + ve ion) m/z; NMR | Comments |
| C32 | 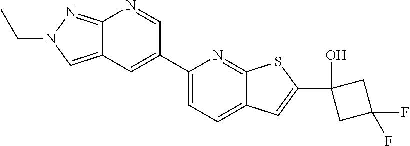 [SMILES: CCn1cc2cc(-c3ccc4cc(C5(O)CC(F)(F)C5)sc4n3)cnc2n1] | 387.2; ¹H NMR (400 MHz, DMSO- d₆) δ 9.40 (d, J = 2.3 Hz, 1H), 8.93 (d, J = 2.4 Hz, 1H), 8.59 (s, 1H), 8.30 (d, J = 8.4 Hz, 1H), 8.12 (d, J = 8.4 Hz, 1H), 7.47 (s, 1H), 6.85 (s, 1H), 4.53 (q, J = 7.3 Hz, 2H), 3.24 (dd, J = 13.4, 9.6 Hz, 2H), 3.06 (q, J= 13.6 Hz, 2H), 1.56 (t, J = 7.3 | |
| | 1-(6-(2-ethyl-2H-pyrazolo[3,4-b]pyridin-5-yl)thieno[2,3- | Hz, 3H) | |
| | b]pyridin-2-yl)-3,3-difluorocyclobutanol | | |
| C34 | 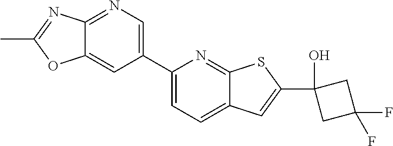 [SMILES: Cc1nc2ncc(-c3ccc4cc(C5(O)CC(F)(F)C5)sc4n3)cc2o1] | 374.0; ¹H NMR (400 MHz, DMSO- d₆) δ 9.28 (d, J = 2.0 Hz, 1H), 8.80 (d, J = 2.0 Hz, 1H), 8.34 (d, J = 8.5 Hz, 1H), 8.18 (d, J = 8.5 Hz, 1H), 7.48 (s, 1H), 6.87 (s, 1H), 3.28- 3.20 (m, 2H), 3.06 (q, J = 13.7 Hz, 2H), 2.74 (s, 3H) | |
| | 3,3-difluoro-1-(6-(2-methyl[1,3]oxazolo[4,5- | | |
| | b]pyridin-6-yl)thieno[2,3-b]pyridin-2-yl)cyclobutanol | | |
| C35 | 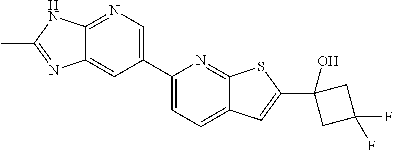 [SMILES: Cc1nc2cc(-c3ccc4cc(C5(O)CC(F)(F)C5)sc4n3)cnc2[nH]1] | 373.2; ¹H NMR (400 MHz, DMSO- d₆) δ 12.75 (s, 1H), 9.03 (d, J = 2.1 Hz, 1H), 8.51 (d, J = 2.1 Hz, 1H), 8.27 (d, J = 8.5 Hz, 1H), 8.10 (d, J = 8.4 Hz, 1H), 7.45 (s, 1H), 6.84 (s, 1H), 3.29-3.16 (m, 2H), 3.05 (q, J = 13.7 Hz, 2H), 2.56 (s, 3H) | |
| | 3,3-difluoro-1-(6-(2-methyl-3H-imidazo[4,5-b]pyridin-6- | | |
| | yl)thieno[2,3-b]pyridin-2-yl)cyclobutanol | | |
| C36 | 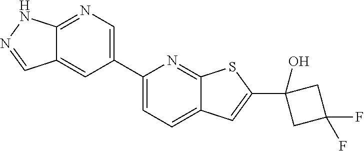 [SMILES: OC1(c2cc3ccc(-c4cnc5[nH]ncc5c4)nc3s2)CC(F)(F)C1] | 359.2; ¹H NMR (401 MHz, DMSO- d₆) δ 13.81 (s, 1H), 9.33 (d, J = 2.2 Hz, 1H), 8.96 (d, J = 2.1 Hz, 1H), 8.31 (d, J = 8.4 Hz, 1H), 8.27 (s, 1H), 8.14 (d, J = 8.4 Hz, 1H), 7.47 (s, 1H), 6.86 (s, 1H), 3.24 (ddt, J = 14.9, 9.1, 4.4 Hz, 2H), 3.15-2.95 (m, 2H) | |
| | 3,3-difluoro-1-(6-(1H-pyrazolo[3,4-b]pyridin-5- | | |
| | yl)thieno[2,3-b]pyridin-2-yl)cyclobutanol | | |
| C37 | 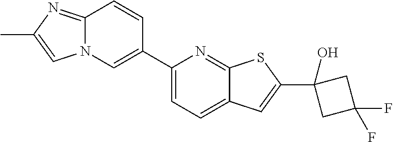 [SMILES: Cc1cn2cc(-c3ccc4cc(C5(O)CC(F)(F)C5)sc4n3)ccc2n1] | 372.2; ¹H NMR (400 MHz, DMSO- d₆) δ 9.33 (dd, J = 1.8, 1.0 Hz, 1H), 8.29 (d, J = 8.4 Hz, 1H), 8.02 (d, J 8.5 Hz, 1H), 7.97 (dd, J = 9.5, 1.9 Hz, 1H), 7.82 (s, 1H), 7.55 (d, J = 9.4 Hz, 1H), 7.46 (s, 1H), 6.85 (s, 1H), 3.30-3.15 (m, 2H), 3.13- 2.94 (m, 2H), 2.37 (d, J = 0.8 Hz, 3H) | |
| | 3,3-difluoro-1-(6-(2-methylimidazo[1,2-a]pyridin-6- | | |
| | yl)thieno[2,3-b]pyridin-2-yl)cyclobutanol | | |
| C38 | 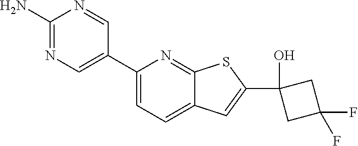 [SMILES: Nc1ncc(-c2ccc3cc(C4(O)CC(F)(F)C4)sc3n2)cn1] | 335.2; ¹H NMR (400 MHz, DMSO- d₆) δ 8.99 (s, 2H), 8.21 (d, J = 8.4 Hz, 1H), 7.92 (d, J = 8.4 Hz, 1H), 7.41 (s, 1H), 7.04 (s, 2H), 6.81 (s, 1H), 3.23 (td, J = 13.5, 9.4 Hz, 2H), 3.04 (q, J = 13.6 Hz, 2H) | |
| | 1-(6-(2-amino-5-pyrimidinyl)thieno[2,3- | | |
| | b]pyridin-2-yl)-3,3-difluorocyclobutanol | | |
| C41 | 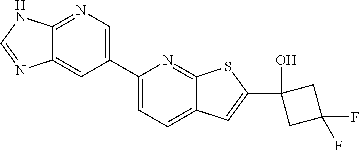 [SMILES: OC1(c2cc3ccc(-c4cnc5[nH]cnc5c4)nc3s2)CC(F)(F)C1] | 359.2; ¹H NMR (400 MHz, DMSO- d₆) δ 9.04 (d, J = 2.2 Hz, 1H), 8.58 (d, J = 2.2 Hz, 1H), 8.32 (s, 1H), 8.25 (d, J = 8.4 Hz, 1H), 8.08 (d, J = 8.4 Hz, 1H), 7.44 (s, 1H), 3.29- 3.21 (m, 2H), 3.05 (td, J = 14.2, 11.7 Hz, 2H) | |
| | 3,3-difluoro-1-(6-(3H-imidazo[4,5-b]pyridin-6- | | |
| | yl)thieno[2,3-b]pyridin-2-yl)cyclobutanol | | |
| C42 | 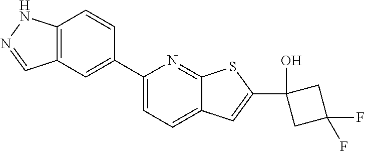 [SMILES: OC1(c2cc3ccc(-c4ccc5[nH]ncc5c4)nc3s2)CC(F)(F)C1] | 358.2; ¹H NMR (400 MHz, DMSO- d₆) δ 13.20 (s, 1H), 8.57 (d, J = 1.7 Hz, 1H), 8.29-8.12 (m, 3H), 8.06 (d, J = 8.5 Hz, 1H), 7.66 (d, J = 8.8 Hz, 1H), 7.44 (s, 1H), 6.82 (s, 1H), 3.23 (dd, J = 13.7, 9.8 Hz, 2H), 3.05 (q, J = 13.6 Hz, 2H) | |
| | 3,3-difluoro-1-(6-(1H-indazol-5-yl)thieno[2,3-b]pyridin-2- | | |
| | yl)cyclobutanol | | |
| C43 | 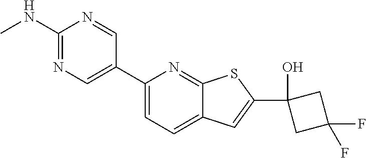 [SMILES: CNc1ncc(-c2ccc3cc(C4(O)CC(F)(F)C4)sc3n2)cn1] | 349.2; ¹H NMR (400 MHz, DMSO- d₆) δ 9.03 (s, 2H), 8.21 (d, J = 8.4 Hz, 1H), 7.91 (d, J = 8.4 Hz, 1H), 7.50 (q, J = 4.9 Hz, 1H), 7.41 (s, 1H), 6.81 (s, 1H), 3.23 (td, J = 13.4, 9.3 Hz, 2H), 3.04 (q, J = 13.7 Hz, 2H), 2.89 (d, J = 4.8 Hz, 3H) | |
| | 3,3-difluoro-1-(6-(2-(methylamino)-5- | | |
| | pyrimidinyl)thieno[2,3-b]pyridin-2-yl)cyclobutanol | | |
| C47 | 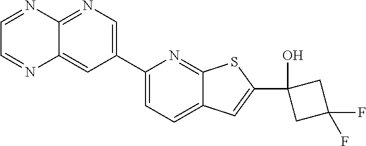 [SMILES: OC1(c2cc3ccc(-c4cnc5nccnc5c4)nc3s2)CC(F)(F)C1] | 371.2; ¹H NMR (400 MHz, DMSO- d₆) δ 9.99 (d, J = 2.4 Hz, 1H), 9.27- 9.11 (m, 3H), 8.42 (s, 2H), 7.54 (d, J = 1.4 Hz, 1H), 6.92 (d, J = 1.3 Hz, 1H), 3.27 (t, J = 11.5 Hz, 2H), 3.08 (q, J = 13.8 Hz, 2H) | |
| | 3,3-difluoro-1-(6-(pyrido[2,3-b]pyrazin-7-yl)thieno[2,3- | | |
| | b]pyridin-2-yl)cyclobutanol | | |
| C51 | 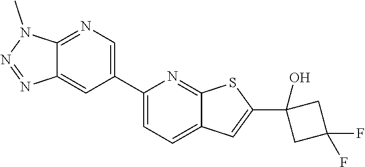 [SMILES: Cn1nnc2cc(-c3ccc4cc(C5(O)CC(F)(F)C5)sc4n3)cnc21] | 374.2; ¹H NMR (400 MHz, DMSO- d₆) δ 9.59 (d, J = 2.0 Hz, 1H), 9.23 (d, J = 2.0 Hz, 1H), 8.37 (d, J = 8.4 Hz, 1H), 8.27 (d, J = 8.4 Hz, 1H), 7.50 (s, 1H), 6.91 (s, 1H), 4.37 (s, 3H), 3.25 (dd, J = 13.8, 9.9 Hz, 2H), 3.07 (q, J = 13.6 Hz, 2H) | Alternate condition (1) was used |
| | 3,3-difluoro-1-(6-(3-methyl-3H-[1,2,3]triazolo[4,5-b]pyridin-6- | | |
| | yl)thieno[2,3-b]pyridin-2-yl)cyclobutanol | | |

### Group 11

Example 11-1:3,3-difluoro-1-(6-(2-methylimidazo[1,2-b]pyridazin-7-yl)thieno[2,3-b]pyridin-2-yl)cyclobutanol C54

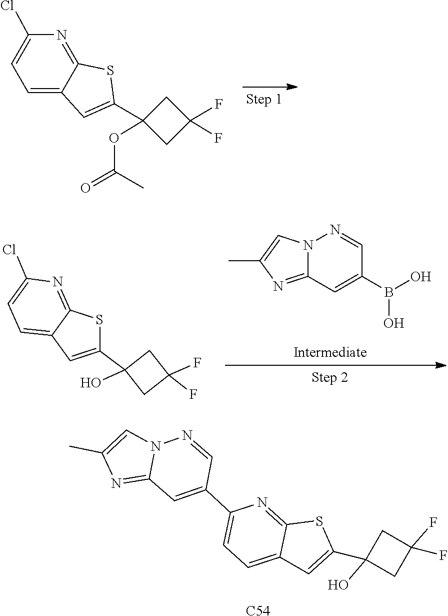

Step 1: 1-(6-chlorothieno[2,3-b]pyridin-2-yl)-3,3-difluorocyclobutan-1-ol. To a solution of 1-(6-chlorothieno[2,3-b]pyridin-2-yl)-3,3-difluorocyclobutyl acetate (0.5 g, 1.57 mmol) in MeOH (15.00 mL) and H₂O (1.50 mL) at rt was added K₂CO₃ (0.652 g, 4.72 mmol), and the mixture was stirred for 1 h. After, the solution was concentrated under reduced pressure, diluted with H₂O (1000 mL) and extracted with EtOAc (100 mL×3). The organic layers were combined, washed with brine (30 mL), dried over Na₂SO₄ and concentrated under reduced pressure to afford crude 1-(6-chlorothieno[2,3-b]pyridin-2-yl)-3,3-difluorocyclobutan-1-ol (0.4 g, 1.16 mmol, 74% yield), which was used directly in the next step without further purification. m/z (ESI): 276.0 (M+H)⁺. ¹H NMR (400 MHz, DMSO-d₆) δ 8.26 (d, J=8.4 Hz, 1H), 7.52 (d, J=8.4 Hz, 1H), 7.48 (s, 1H), 6.91 (s, 1H), 3.29-3.17 (m, 2H), 3.04 (td, J=14.2, 12.0 Hz, 2H).

Step 2: 3,3-difluoro-1-(6-(2-methylimidazo[1,2-b]pyridazin-7-yl)thieno[2,3-b]pyridin-2-yl)cyclobutan-1-ol. A stirred solution of crude 1-(6-chlorothieno[2,3-b]pyridin-2-yl)-3,3-difluorocyclobutan-1-ol (0.15 g, 0.54 mmol), Intermediate 6 (0.106 g, 0.60 mmol) and K₂CO₃ (0.226 g, 1.63 mmol) in 1,4-dioxane (5.40 mL) and H₂O (0.60 mL) was purged with N₂ gas for 5 min. After purging, SPhos Pd G3 (0.042 g, 0.054 mmol) was added, and the mixture was stirred at 110° C. for 3 h. After, the mixture was diluted with EtOAc, filtered, and washed with EtOAc (50 mL). The filtrate was concentrated under reduced pressure to afford a crude material. The crude material was purified by reverse-phase preparative HPLC, eluting with a linear gradient of (0.1% NH₃)H₂O:ACN and a flow rate of 15 mL/min, to provide Compound C54 (0.096 g, 0.26 mmol, 47% yield). m/z (ESI): 373.2 (M+H)⁺. ¹H NMR (400 MHz, DMSO-d₆) δ 9.28 (d, J=2.1 Hz, 1H), 8.71 (d, J=2.1 Hz, 1H), 8.34 (d, J=8.4 Hz, 1H), 8.27 (d, J=8.4 Hz, 1H), 8.17 (s, 1H), 7.49 (s, 1H), 6.91 (s, 1H), 3.31-3.19 (m, 2H), 3.06 (q, J=13.6 Hz, 2H), 2.44 (s, 3H).

Compounds in Table 11-1 were prepared following the procedure described in Group 11, using appropriate starting materials (e.g., aldehyde/ketones and boronic esters). All starting materials are commercially available or are described in the Intermediates section above.

TABLE 11-1

| | | | Reaction |
|---|---|---|---|
| Ex. # | Chemical Structure & Name | LCMS: (ESI + ve ion) m/z; NMR | Comments |
| C39 | 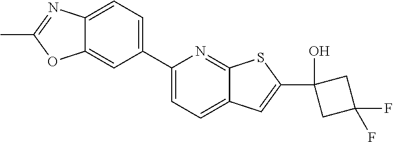 [SMILES: Cc1nc2ccc(-c3ccc4cc(C5(O)CC(F)(F)C5)sc4n3)cc2o1] | 373.2; ¹H NMR (400 MHz, DMSO- d₆) δ 8.42 (d, J = 1.6 Hz, 1H), 8.29 (d, J = 8.4 Hz, 1H), 8.19 (dd, J = 8.4, 1.6 Hz, 1H), 8.11 (d, J = 8.4 Hz, 1H), 7.77 (d, J = 8.4 Hz, 1H), 7.46 (s, 1H), 6.84 (s, 1H), 3.29- 3.19 (m, 2H), 3.05 (q, J = 13.6 Hz, 2H), 2.67 (s, 3H) | |
| | 3,3-difluoro-1-(6-(2-methyl-1,3-benzoxazol-6-yl)thieno[2,3- | | |
| | b]pyridin-2-yl)cyclobutanol | | |
| C44 | 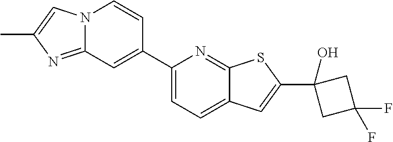 [SMILES: Cc1cn2ccc(-c3ccc4cc(C5(O)CC(F)(F)C5)sc4n3)cc2n1] | 372.2; ¹H NMR (400 MHz, DMSO- d₆) δ 8.58 (d, J = 7.1 Hz, 1H), 8.33- 8.22 (m, 2H), 8.18 (d, J = 8.4 Hz, 1H), 7.79 (s, 1H), 7.74 (dd, J = 7.1, 1.8 Hz, 1H), 7.47 (s, 1H), 6.86 (s, 1H), 3.24 (dd, J = 13.7, 9.8 Hz, 2H), 3.06 (dt, J = 20.6, 13.0 Hz, 2H), 2.39 (s, 3H) | Intermediate 7 was used |
| | 3,3-difluoro-1-(6-(2-methylimidazo[1,2-a]pyridin-7- | | |
| | yl)thieno[2,3-b]pyridin-2-yl)cyclobutanol | | |
| C46 | 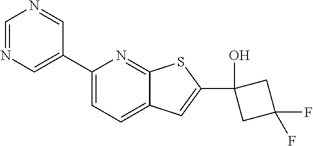 [SMILES: OC1(c2cc3ccc(-c4cncnc4)nc3s2)CC(F)(F)C1] | 320.2; ¹H NMR (400 MHz, DMSO- d₆) δ 9.52 (d, J = 1.2 Hz, 2H), 9.27 (d, J = 1.4 Hz, 1H), 8.37 (dd, J = 8.4, 1.3 Hz, 1H), 8.18 (dd, J = 8.4, 1.4 Hz, 1H), 7.51 (d, J = 1.4 Hz, 1H), 6.90 (s, 1H), 3.25 (ddd, J = 12.7, 7.3, 3.6 Hz, 2H), 3.06 (td, J = 14.4, 12.1 Hz, 2H) | |
| | 3,3-difluoro-1-(6-(5-pyrimidinyl)thieno[2,3- | | |
| | b]pyridin-2-yl)cyclobutanol | | |
| C49 | 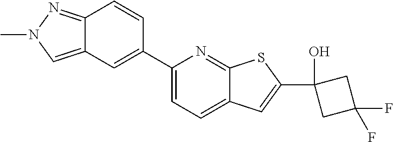 [SMILES: Cn1cc2cc(-c3ccc4cc(C5(O)CC(F)(F)C5)sc4n3)ccc2n1] | 372.2; ¹H NMR (400 MHz, DMSO- d₆) δ 8.52 (s, 1H), 8.47 (s, 1H), 8.24 (dd, J = 8.5, 2.2 Hz, 1H), 8.14- 7.99 (m, 2H), 7.70 (d, J = 9.3 Hz, 1H), 7.44 (d, J = 2.2 Hz, 1H), 6.81 (d, J = 2.3 Hz, 1H), 4.21 (d, J = 2.2 Hz, 3H), 3.28-3.19 (m, 2H), 3.05 (q, J = 13.5, 13.1 Hz, 2H) | |
| | 3,3-difluoro-1-(6-(2-methyl-2H-indazol-5-yl)thieno[2,3- | | |
| | b]pyridin-2-yl)cyclobutanol | | |

### Group 12

Example 12-1: (R)-(3,3-difluorocyclobutyl)(2-(2-methyl-2H-pyrazolo[3,4-b]pyridin-5-yl)thieno[2,3-d]pyrimidin-6-yl)methanol B75R; (S)-(3,3-difluorocyclobutyl)(2-(2-methyl-2H-pyrazolo[3,4-b]pyridin-5-yl)thieno[2,3-d]pyrimidin-6-yl)methanol B75S

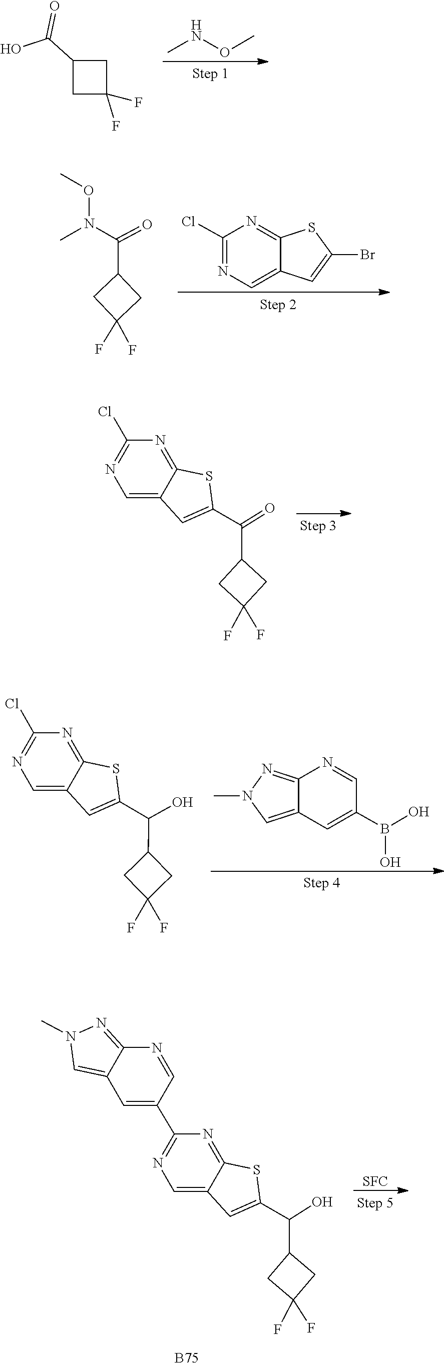

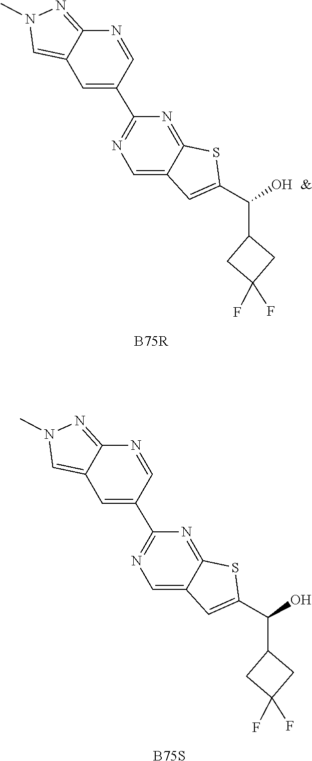 [SMILES: "B75R" ; "B75S" ; Cn1cc2cc(-c3ncc4cc([C@H](O)C5CC(F)(F)C5)sc4n3)cnc2n1 ; Cn1cc2cc(-c3ncc4cc([C@@H](O)C5CC(F)(F)C5)sc4n3)cnc2n1]

Step 1: 3,3-difluoro-N-methoxy-N-methylcyclobutane-1-carboxamide. To a flask was added 3,3-difluorocyclobutane-1-carboxylic acid (1.00 g, 7.35 mmol), diisopropylamine (6.42 mL, 36.7 mmol), HATU (8.38 g, 22.04 mmol), and N,O-dimethylhydroxylamine hydrochloride (2.150 g, 22.04 mmol) in DCM (10 mL). The reaction mixture was stirred at rt for 16 h. The reaction mixture was diluted with H₂O (10 mL) and extracted with DCM (2×10 mL). The organic extracts were washed with brine (10 mL) and dried over Na₂SO₄. The mixture was filtered and concentrated under reduced pressure to give a crude material. The crude material purified by chromatography, eluting with a gradient of 1% to 30% EtOAc in hexane, to provide 3,3-difluoro-N-methoxy-N-methylcyclobutane-1-carboxamide (0.6 g, 3.35 mmol, 46% yield). m/z (ESI): 180.1 (M+H)⁺.

Step 2: (2-chlorothieno[2,3-d]pyrimidin-6-yl)(3,3-difluorocyclobutyl)meth anone. A solution of 6-bromo-2-chlorothieno[2,3-d]pyrimidine (1 g, 4.01 mmol) and 3,3-difluoro-N-methoxy-N— methylcyclobutane-1-carboxamide (1.436 g, 8.02 mmol) in THF (20 mL) was stirred at −78° C. for 10 min. Then, at the same temperature, n-butyllithium (2.5 M in hexanes, 4.01 mL, 10.02 mmol) was added, and the reaction mixture was stirred at −78° C. for 30 min. Next, the mixture was quenched by addition of satd. aq. NH₄Cl (20 mL), extracted with EtOAc (2×50 mL), dried over Na₂SO₄, filtered, and concentrated under reduced pressure to give a crude material. The crude material was purified by chromatography, eluting with a gradient of 1% to 40% EtOAc in hexane, to provide (2-chlorothieno[2,3-d]pyrimidin-6-yl)(3,3-difluorocyclobutyl)methanone (290 mg, 0.90 mmol, 23% yield). m/z (ESI): 289.2 (M+H)⁺.

Step 3: (2-chlorothieno[2,3-d]pyrimidin-6-yl)(3,3-difluorocyclobutyl)methanol. A solution of (2-chlorothieno[2,3-d]pyrimidin-6-yl)(3,3-difluorocyclobutyl)methanone (300 mg, 1.04 mmol) in THF (9 mL) and EtOH (1 mL) was cooled to −78° C. and NaBH₄ (79 mg, 2.08 mmol) was added. Then, the reaction was stirred at −78° C. for 15 min. After, the reaction mixture was quenched by addition of H₂O (10 mL), concentrated, and diluted with H₂O (10 mL). Then, the mixture was extracted with EtOAc (2×25 mL), washed with brine (25 mL), dried over Na₂SO₄, filtered, and concentrated under reduced pressure to provide a crude material. The crude material was purified by chromatography, eluting with a gradient of 1% to 60% EtOAc in hexane, to provide (2-chlorothieno[2,3-d]pyrimidin-6-yl)(3,3-difluorocyclobutyl)methanol (200 mg, 0.69 mmol, 66% yield). m/z (ESI): 291.2 (M+H)⁺. ¹H NMR (401 MHz, DMSO-d₆) δ 9.16 (s, 1H), 7.46 (s, 1H), 6.47 (d, J=5.2 Hz, 1H), 4.98 (t, J=4.5 Hz, 1H), 2.68-2.55 (m, 4H).

Step 4: (3,3-difluorocyclobutyl)(2-(2-methyl-2H-pyrazolo[3,4-b]pyridin-5-yl)thieno[2,3-d]pyrimidin-6-yl)methanol. To a solution of (2-chlorothieno[2,3-d]pyrimidin-6-yl)(3,3-difluorocyclobutyl)methanol (250 mg, 0.86 mmol) in 1,4-dioxane (10 mL) and H₂O (2 mL) was added (2-methyl-2H-pyrazolo[3,4-b]pyridin-5-yl)boronic acid (198 mg, 1.12 mmol) and K₂CO₃ (357 mg, 2.58 mmol), and the mixture was degassed with N₂ for 5 min. Then, SPhos Pd G3 (671 mg, 0.86 mmol) was added, and the reaction mixture was stirred at 90° C. for 12 h. The reaction mixture was diluted with H₂O (10 mL), extracted with EtOAc (2×50 mL), washed with brine (50 mL), and dried over Na₂SO₄. Then, the mixture was filtered and concentrated under reduced pressure to provide crude material. The crude material was purified by reverse-phase preparative HPLC, eluting with 0% to 70% of (0.1% NH₃) H₂O:ACN, to provide Compound B75 (250 mg, 0.65 mmol, 75% yield). m/z (ESI): 388.2 (M+H)⁺. ¹H NMR (401 MHz, DMSO-d₆) δ 9.63 (d, J=2.2 Hz, 1H), 9.31 (s, 1H), 9.22 (d, J=2.2 Hz, 1H), 8.59 (s, 1H), 7.46 (s, 1H), 6.41 (d, J=5.2 Hz, 1H), 4.99 (t, J=5.0 Hz, 1H), 4.25 (s, 3H), 2.65-2.56 (m, 5H).

Step 5: SFC Purification. The sample was purified via SFC using a Cellulose-SZ 250×4.6 mm, 5 μm column with a mobile phase of 50% (1:1) IPA:ACN in CO₂ with a flow rate of 4.0 mL/min to generate peak 1 and peak 2. Peak 1: B75R. (100 mg, 0.26 mmol, 40% yield). m/z (ESI): 388.2 (M+H)⁺. ¹H NMR (401 MHz, DMSO-d₆) δ 9.63 (d, J=2.2 Hz, 1H), 9.31 (s, 1H), 9.22 (d, J=2.2 Hz, 1H), 8.59 (s, 1H), 7.46 (d, J=1.1 Hz, 1H), 6.41 (d, J=5.1 Hz, 1H), 4.99 (t, J=4.9 Hz, 1H), 4.25 (s, 3H), 2.64-2.55 (m, 5H).

Peak 2: B75S. (70 mg, 0.18 mmol, 28% yield). m/z (ESI): 388.2 (M+H)⁺. ¹H NMR (401 MHz, DMSO-d₆) δ 9.63 (d, J=2.2 Hz, 1H), 9.31 (s, 1H), 9.22 (d, J=2.2 Hz, 1H), 8.59 (s, 1H), 7.46 (d, J=1.1 Hz, 1H), 6.41 (d, J=5.1 Hz, 1H), 4.99 (t, J=4.9 Hz, 1H), 4.25 (s, 3H), 2.64-2.55 (m, 5H).

### Alternate Conditions:

(1) After Step 3: To a stirred solution at rt of the product of Step 3 (0.483 mmol), 4-nitrobenzoic acid (0.121 g, 0.725 mmol) and triphenylphosphine (0.190 g, 0.725 mmol) in THF (8.40 mL) was added a solution of diethyl azodicarboxylate (0.115 mL, 0.725 mmol) in THF (1 mL). The reaction mixture was stirred at rt for 48 h. The reaction mass was diluted with EtOAc and washed with satd. aq. NaHCO₃. The organic layer was dried over Na₂SO₄, filtered and concentrated in vacuo. The crude material was purified by column chromatography, eluting with 10% EtOAc in pet. ether, to afford product.
(2) Prior to Step 4: To a stirred 0° C. solution of the product of Alternate condition (1) (0.160 mmol) in methanol (3.5 mL) was added sodium hydroxide (0.013 g, 0.319 mmol) in water (0.1 mL). The reaction mixture was stirred at rt for 30 min. The reaction mass was concentrated to provide crude material. The crude material was diluted with EtOAc and washed with water. The organic layer was separated and concentrated to afford product.
(3) Step 2 was performed in toluene instead of THF.

Compounds in Table 12-1 were prepared following the procedure described in Group 12, using appropriate starting materials (e.g., aldehyde/ketones and boronic esters). All starting materials are commercially available or are described in the Intermediates section above.

TABLE 12-1

| Ex. # | Chemical Structure & Name | LCMS: (ESI + ve ion) m/z; NMR | Reaction Comments |
|---|---|---|---|
| B78S | 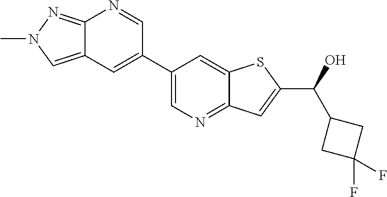 [SMILES: Cn1cc2cc(-c3cnc4cc([C@@H](O)C5CC(F)(F)C5)sc4c3)cnc2n1] | 387.1; ¹H NMR (400 MHz, DMSO- d₆) δ 9.02-8.98 (m, 2H), 8.80 (d, J = 2.2 Hz, 1H), 8.59 (d, J = 2.4 Hz, 1H), 8.53 (s, 1H), 7.48 (s, 1H), 6.32 (d, J = 5.1 Hz, 1H), 5.01 (s, 1H), 4.25 (s, 3H), 2.60-2.55 (m, 5H) | Peak 1/SFC: Chiralpak IC-3, 250 × 4.6 mm 3 μm; Mobile phase: 40% (1:1) MeOH: ACN in CO₂ using a flow rate of 5.0 mL/min. |
| | (S)-(3,3-difluorocyclobutyl)(6-(2-methyl-2H-pyrazolo[3,4- | | |
| | b]pyridin-5-yl)thieno[3,2-b]pyridin-2-yl)methanol | | |
| B78R | 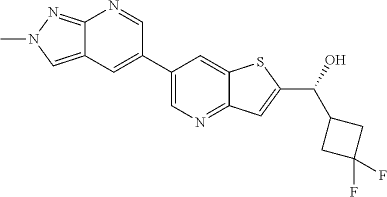 [SMILES: Cn1cc2cc(-c3cnc4cc([C@H](O)C5CC(F)(F)C5)sc4c3)cnc2n1] | 387.1; ¹H NMR (400 MHz, DMSO- d₆) δ 9.04-8.98 (m, 2H), 8.80 (s, 1H), 8.59 (d, J = 2.4 Hz, 1H), 8.53 (s, 1H), 7.48 (s, 1H), 6.32 (d, J = 5.1 Hz, 1H), 5.01 (s, 1H), 4.25 (s, 3H), 2.60-2.55 (m, 5H) | Peak 2/SFC: Chiralpak IC-3, 250 × 4.6 mm 3 μm; Mobile phase: 40% (1:1) MeOH: ACN in CO₂ using a flow rate of 5.0 mL/min. |
| | (R)-(3,3-difluorocyclobutyl)(6-(2-methyl-2H-pyrazolo[3,4- | | |
| | b]pyridin-5-yl)thieno[3,2-b]pyridin-2-yl)methanol | | |
| B94RS | 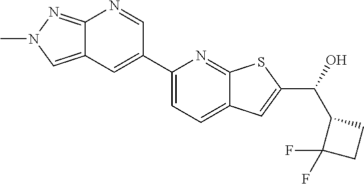 [SMILES: Cn1cc2cc(-c3ccc4cc([C@H](O)[C@@H]5CCC5(F)F)sc4n3)cnc2n1] | 387.0; ¹H NMR (400 MHz, DMSO- d₆) δ 9.39 (d, J = 2.3 Hz, 1H), 8.92 (d, J = 2.3 Hz, 1H), 8.55 (s, 1H), 8.30 (d, J = 8.5 Hz, 1H), 8.10 (d, J = 8.4 Hz, 1H), 7.32 (s, 1H), 6.24 (s, 1H), 5.09 (d, J = 8.2 Hz, 1H), 4.25 (s, 3H), 3.27-3.06 (m, 1H), 2.50- 2.46 (m, 2H), 1.98 (d, J = 9.6 Hz, 1H), 1.93-1.80 (m, 1H) | Alternate conditions (1), (2), and (3) were used; SFC was performed after Step 2; Peak 2/SFC: Chiralpak IG 250 x 50 mm, 5 μm; Mobile phase: 40% (1:1) MeOH:ACN in CO₂ with a flow rate |
| | (R)-((1S)-2,2-difluorocyclobutyl)(6-(2-methyl- | | of 150 mL/min |
| | 2H-pyrazolo[3,4-b]pyridin-5-yl)thieno[2,3-b]pyridin-2- | | |
| | yl)methanol | | |
| B94SR | 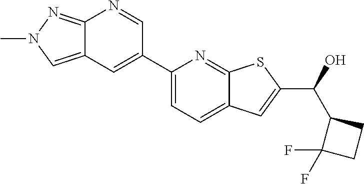 [SMILES: Cn1cc2cc(-c3ccc4cc([C@@H](O)[C@H]5CCC5(F)F)sc4n3)cnc2n1] | 387.2; ¹H NMR (400 MHz, DMSO- d₆) δ 9.39 (d, J = 2.4 Hz, 1H), 8.92 (d, J = 2.3 Hz, 1H), 8.55 (s, 1H), 8.30 (d, J = 8.4 Hz, 1H), 8.10 (d, J = 8.4 Hz, 1H), 7.32 (s, 1H), 6.24 (s, 1H), 5.09 (d, J = 8.1 Hz, 1H), 4.25 (s, 3H), 3.17 (m, J = 16.7, 10.5, 6.2 Hz, 1H), 2.47 (dd, J = 9.7, 6.9 Hz, 2H), 1.98 (d, J = 6.6 Hz, 1H), 1.93- 1.81 (m, 1H) | Alternate conditions (1), (2), and (3) were used; SFC was performed after Step 2; Peak 1/SFC: Chiralpak IG 250 x 50 mm, 5 μm; Mobile phase: 40% (1:1) MeOH:ACN in CO₂ with a flow rate |
| | (S)-((1R)-2,2-difluorocyclobutyl)(6-(2-methyl- | | of 150 mL/min |
| | 2H-pyrazolo[3,4-b]pyridin-5-yl)thieno[2,3-b]pyridin-2- | | |
| | yl)methanol | | |
| B94RR | 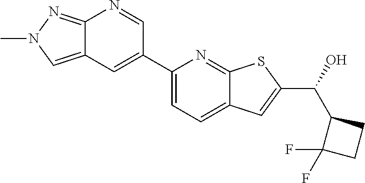 [SMILES: Cn1cc2cc(-c3ccc4cc([C@H](O)[C@H]5CCC5(F)F)sc4n3)cnc2n1] | 387.2; ¹H NMR (400 MHz, DMSO-d₆) δ 9.38 (d, J = 2.3 Hz, 1H), 8.92 (d, J = 2.4 Hz, 1H), 8.55 (s, 1H), 8.28 (d, J = 8.5 Hz, 1H), 8.10 (d, J = 8.4 Hz, 1H), 7.37 (d, J = 0.7 Hz, 1H), 6.22 (d, J = 5.1 Hz, 1H), 5.06 (dd, J = 9.8, 5.1 Hz, 1H), 4.24 (s, 3H), 3.12 (q, J = 11.1 Hz, 1H), 2.47-2.35 (m, 2H), 1.71 (m, J = 10.1, 5.0 Hz, 1H), 1.57 (q, J = 9.5 Hz, 1H) | Alternative condition (3) was used; SFC was performed after Step 3; Peak 2/SFC: Chiralcel OJ-H 250 x 30 mm; Mobile phase: 15% (1:1) MeOH:ACN in CO₂ with a flow rate of 100 mL/min |
| | (R)-((1R)-2,2-difluorocyclobutyl)(6-(2-methyl- | | |
| | 2H-pyrazolo[3,4-b]pyridin-5-yl)thieno[2,3-b]pyridin-2- | | |
| | yl)methanol | | |
| B94SS | 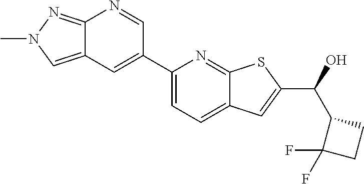 [SMILES: Cn1cc2cc(-c3ccc4cc([C@@H](O)[C@@H]5CCC5(F)F)sc4n3)cnc2n1] | 387.2; ¹H NMR (400 MHz, DMSO- d₆) δ 9.38 (d, J = 2.3 Hz, 1H), 8.92 (d, J = 2.4 Hz, 1H), 8.55 (s, 1H), 8.28 (d, J = 8.4 Hz, 1H), 8.10 (d, J = 8.4 Hz, 1H), 7.37 (d, J = 0.8 Hz, 1H), 6.22 (d, J = 5.1 Hz, 1H), 5.11- 4.96 (m, 1H), 4.24 (s, 3H), 3.11 (m, J = 21.5, 10.3 Hz, 1H), 2.49- 2.27 (m, 2H), 1.71 (dd, J = 10.0, 5.2 Hz, 1H), 1.56 (p, J = 9.8 Hz, 1H) | Alternative condition (3) was used; SFC was performed after Step 3; Peak 1/SFC: Chiralcel OJ-H 250 x 30 mm; Mobile phase: 15% (1:1) MeOH:ACN in CO₂ with a flow rate of 100 mL/min |
| | (S)-((1S)-2,2-difluorocyclobutyl)(6-(2-methyl- | | |
| | 2H-pyrazolo[3,4-b]pyridin-5-yl)thieno[2,3-b] | | |
| | pyridin-2-yl)methanol | | |

### Group 13

Example 13-1: (R)-(3-chloro-6-(2-methyl-2H-pyrazolo[3,4-b]pyridin-5-yl)thieno[2,3-b]pyridin-2-yl)(3,3-difluorocyclobutyl)methanol B76R; (S)-(3-chloro-6-(2-methyl-2H-pyrazolo[3,4-b]pyridin-5-yl)thieno[2,3-b]pyridin-2-yl)(3,3-difluorocyclobutyl)methanol B76S

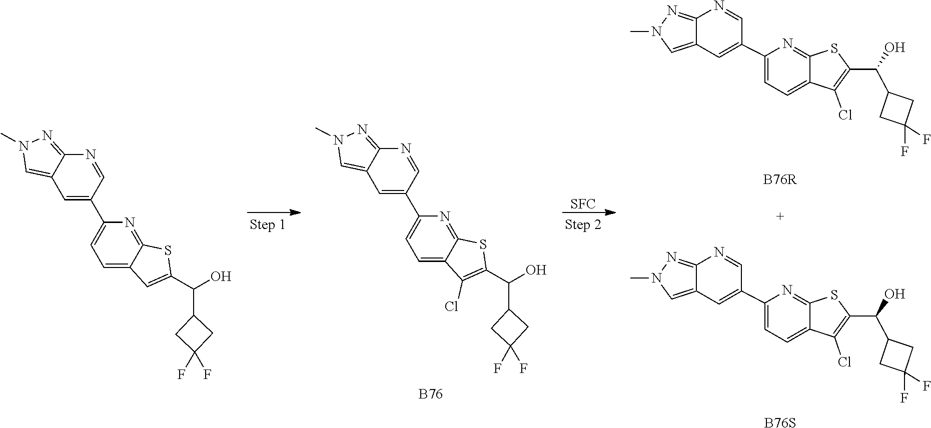

Step 1: (3-chloro-6-(2-methyl-2H-pyrazolo[3,4-b]pyridin-5-yl)thieno[2,3-b]pyridin-2-yl)(3,3-difluorocyclobutyl)methanol, B76. To a solution of (3,3-difluorocyclobutyl)(6-(2-methyl-2H-pyrazolo[3,4-b]pyridin-5-yl)thieno[2,3-b]pyridin-2-yl)methanol (1.00 g, 2.59 mmol) in acetic acid (50.0 mL) was added N-chlorosuccinimide (0.415 g, 3.11 mmol), and then the reaction mixture was stirred at 25° C. for 12 h. The reaction mixture was concentrated in vacuo, diluted with H₂O (25 mL) and extracted with EtOAc (2×25 mL). The combined organic extracts were washed with satd. aq. NaHCO₃ (25 mL), dried over Na₂SO₄, filtered and concentrated to give the crude material. The crude material was purified by preparative HPLC, eluting with a linear gradient of (0.1% NH₃) H₂O:ACN and a flow rate of 15 mL/min, to provide (3-chloro-6-(2-methyl-2H-pyrazolo[3,4-b]pyridin-5-yl)thieno[2,3-b]pyridin-2-yl)(3,3-difluorocyclobutyl)methanol (0.25 g, 0.59 mmol, 23% yield). m/z (ESI): 421.0 (M+H)⁺. ¹H NMR (400 MHz, DMSO-d₆) δ 9.40 (d, J=2.3 Hz, 1H), 8.97 (d, J=2.3 Hz, 1H), 8.57 (s, 1H), 8.27-8.23 (m, 2H), 6.55 (d, J=4.6 Hz, 1H), 5.17 (d, J=4.5 Hz, 1H), 4.25 (s, 3H), 2.78-2.70 (m, 1H), 2.61-2.54 (n, 4H).

Step 2: SFC Purification. The sample was purified by SFC using a Chiralcel 01—H 250×30 mm, 5.m column, with a mobile phase of 40% (1:1) MeOH:ACN in CO₂ and using a flow rate of 120 mL/min to provide peak 1 and peak 2.

Peak 1: B76S (70 mg, 0.17 mmol, 35% o yield). m/z (ESI): 421.2 (M+H)⁺. ¹H NMR (401 MHz, DMSO-d₆) δ 9.40 (d, J=2.3 Hz, 1H), 8.98 (d, J=2.4 Hz, 1H), 8.58 (s, 1H), 8.27-8.23 (m, 2H), 6.56 (s, 1H), 5.17 (d, J=4.1 Hz, 1H), 4.25 (s, 3H), 2.80-2.69 (i, 1H), 2.61-2.54 (, 4H).

Peak 2: B76R (60 mg, 0.14 mmol, 30% yield). m/z(ESI): 421.2 (M+H)⁺. ¹H NMR (401 MHz, DMSO-d₆) δ 9.40 (d, J=2.3 Hz, 1H), 8.98 (d, J=2.4 Hz, 1H), 8.58 (s, 1H), 8.27-8.23 (m, 2H), 6.56 (s, 1H), 5.17 (d,J14.0 Hz, 1H), 4.25 (s, 3H), 2.80-2.69 (, 1H), 2.61-2.54 (n, 4H).

Compounds in Table 13-1 were prepared following the procedure described in Group 13, using appropriate starting materials (e.g., aldehyde/ketones and boronic esters). All starting materials are commercially available or are described in the Intermediates section above.

TABLE 13-1

| | | | Reaction |
|---|---|---|---|
| Ex. # | Chemical Structure & Name | LCMS: (ESI + ve ion) m/z; NMR | Comments |
| C69C | 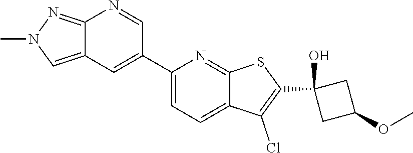 | 401.2; ¹H NMR (400 MHz, ) δ 9.40 (d, J = 2.3 Hz, 1H), 8.97 (d, J = 2.3 Hz, 1H), 8.57 (s, 1H), 8.28-8.16 (m, 2H), 6.49 (s, 1H), 4.25 (s, 3H), 3.86 (p, J = 6.7 Hz, 1H), 3.23-3.07 (m, 5H), 2.47-2.34 (m, 2H) | |
| | cis-1-(3-chloro-6-(2-methyl-2H-pyrazolo[3,4-b]pyridin-5- | | |
| | yl)thieno[2,3-b]pyridin-2-yl)-3-methoxycyclobutanol | | |
| C73 | 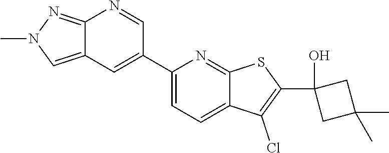 | 399.2; ¹H NMR (400 MHz, ) δ 9.40 (d, J = 2.4 Hz, 1H), 8.97 (d, J = 2.4 Hz, 1H), 8.57 (s, 1H), 8.27-8.17 (m, 2H), 6.31 (s, 1H), 4.25 (s, 3H), 2.77-2.69 (m, 2H), 2.30-2.23 (m, 2H), 1.35 (s, 3H), 1.18 (s, 3H) | |
| | 1-(3-chloro-6-(2-methyl-2H-pyrazolo[3,4-b]pyridin-5- | | |
| | yl)thieno[2,3-b]pyridin-2-yl)-3,3-dimethylcyclobutanol | | |
| C75C | 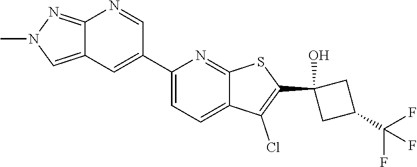 [SMILES: Cn1cc2cc(-c3ccc4c(Cl)c([C@]5(O)C[C@@H](C(F)(F)F)C5)sc4n3)cnc2n1] | 439.0; ¹H NMR (401 MHz, DMSO- d₆) δ 9.41 (d, J = 2.3 Hz, 1H), 8.99 (d, J = 2.4 Hz, 1H), 8.58 (s, 1H), 8.30-8.25 (m, 2H), 6.79 (s, 1H), 4.25 (s, 3H), 3.25-3.16 (m, 1H), 3.07 (ddd, J = 11.4, 8.7, 2.6 Hz, 2H), 2.66-2.56 (m, 2H) | |
| | cis-1-(3-chloro-6-(2-methyl-2H-pyrazolo[3,4-b]pyridin-5- | | |
| | yl)thieno[2,3-b]pyridin-2-yl)-3-(trifluoromethyl)cyclobutanol | | |

### Group 14

Example 14-1:3,3-difluoro-1-(6-(2-methylimidazo[1,2-a]pyrimidin-6-yl)thieno[2,3-b]pyridin-2-yl)cyclobutanol C45

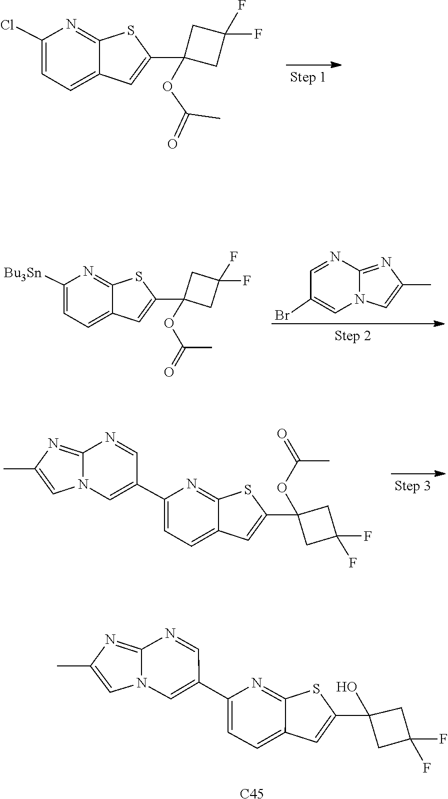

Step 1: 3,3-difluoro-1-(6-(tributylstannyl)thieno[2,3-b]pyridin-2-yl)cyclobutyl acetate. To a 50 mL sealed tube was added 1-(6-chlorothieno[2,3-b]pyridin-2-yl)-3,3-difluorocyclobutyl acetate (0.3 g, 0.94 mmol), lithium chloride (0.240 g, 5.67 mmol) and 1,1,1,2,2,2-hexabutyldistannane (0.657 g, 1.13 mmol) in 1,4-dioxane (9.00 mL) at rt. The mixture was degassed with N₂ gas for 5 min, followed by addition of PCy₃ (0.026 g, 0.094 mmol) and Pd₂(dba)₃ (0.043 g, 0.047 mmol) at rt. The reaction was heated to 120° C. for 16 h. Then, the reaction mixture was filtered, washed with EtOAc and concentrated under reduced pressure to afford a crude material. The crude material was purified by chromatography, eluting with a gradient of 5%-10% EtOAc in hexane, to provide 3,3-difluoro-1-(6-(tributylstannyl)thieno[2,3-b]pyridin-2-yl)cyclobutyl acetate (0.25 g, 0.44 mmol, 46% yield). m/z (ESI): 574.2 (M+H)⁺. ¹H NMR (400 MHz, DMSO-d₆) δ 8.04 (d, J=7.6 Hz, 1H), 7.54-7.49 (m, 2H), 3.59-3.32 (m, 4H), 2.08 (s, 3H), 1.69-1.44 (m, 6H), 1.37-1.29 (m, 6H), 1.21-1.07 (m, 6H), 0.91-0.81 (m, 9H).

Step 2: 3,3-difluoro-1-(6-(2-methylimidazo[1,2-a]pyrimidin-6-yl)thieno[2,3-b]pyridin-2-yl)cyclobutyl acetate. A stirred solution of 3,3-difluoro-1-(6-(tributylstannyl)thieno[2,3-b]pyridin-2-yl)cyclobutyl acetate (0.175 g, 0.31 mmol) and 6-bromo-2-methylimidazo[1,2-a]pyrimidine (0.05 g, 0.24 mmol) in DMF (3.00 mL) was purged with N₂ gas for 5 min. After purging, 1,4-bis(diphenylphosphino)butanepalladium(II) chloride (0.014 g, 0.024 mmol) was added, and the reaction mixture was stirred at 110° C. for 16 h. After, the mixture was quenched by addition of H₂O (50 mL), extracted with EtOAc (30 mL×3), washed with brine (30 mL), dried over Na₂SO₄, and concentrated under reduced pressure to afford crude 3,3-difluoro-1-(6-(2-methylimidazo[1,2-a]pyrimidin-6-yl)thieno[2,3-b]pyridin-2-yl)cyclobutyl acetate (0.03 g, 0.072 mmol, 31% yield), which was used for the next step without further purification. m/z (ESI): 415.0 (M+H)⁺.

Step 3: 3,3-difluoro-1-(6-(2-methylimidazo[1,2-a]pyrimidin-6-yl)thieno[2,3-b]pyridin-2-yl)cyclobutanol. To a solution of crude 3,3-difluoro-1-(6-(2-methylimidazo[1,2-a]pyrimidin-6-yl)thieno[2,3-b]pyridin-2-yl)cyclobutyl acetate (0.3 g, 0.72 mmol) in MeOH (9.00 mL) and H₂O (0.90 mL) was added K₂CO₃ (0.300 g, 2.17 mmol) at rt, and the reaction was stirred for 1 h. After, the solution was concentrated under reduced pressure, diluted with EtOAc, filtered, washed with EtOAc (50 mL), and concentrated under reduced pressure to afford a crude material. The crude material was purified by prep-HPLC, eluting with a linear gradient of (0.1% NH₃) H₂O:ACN with a flow rate of 15 mL/min, to provide Compound C45 (0.03 g, 0.081 mmol, 11% yield). m/z (ESI): 373.2 (M+H)⁺. ¹H NMR (400 MHz, DMSO-d₆) δ 9.68 (d, J=2.5 Hz, 1H), 9.22 (d, J=2.5 Hz, 1H), 8.34 (d, J=8.4 Hz, 1H), 8.11 (d, J=8.4 Hz, 1H), 7.79 (s, 1H), 7.48 (s, 1H), 6.88 (s, 1H), 3.24 (dd, J=13.5, 9.8 Hz, 2H), 3.18-3.01 (m, 2H), 2.41 (s, 3H).

### Alternate Conditions:

(1) Deprotection: Following Step 2, where Intermediate 4 was used, to a stirred solution of the product from Step 2 (0.38 mmol) in THF (16.0 mL), was added 36% HCl (5 mL, 59.2 mmol). The reaction mixture was stirred at rt for 3 h. After, reaction mass was quenched by addition of satd. aq. NaHCO₃ solution (100 mL) and extracted with EtOAc (50 mL×3). The combined organic layers were washed with brine (30 mL), dried over Na₂SO₄, and concentrated under reduced pressure to afford the desired product.

The compound in Table 14-1 was prepared following the procedure described in Group 14, using appropriate starting materials (e.g., aldehyde/ketones and boronic esters). All starting materials are commercially available or are described in the Intermediates section above.

TABLE 14-1

| | | | Reaction |
|---|---|---|---|
| Ex. # | Chemical Structure & Name | LCMS: (ESI + ve ion) m/z; NMR | Comments |
| C55 | 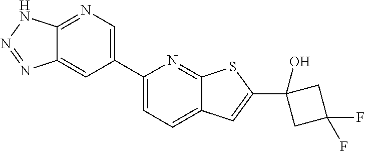 [SMILES: OC1(c2cc3ccc(-c4cnc5[nH]nnc5c4)nc3s2)CC(F)(F)C1] | 360.2; ¹H NMR (400 MHz, DMSO- d₆) δ 9.13 (d, J = 2.1 Hz, 1H), 8.73 (d, J = 2.1 Hz, 1H), 8.25 (d, J = 8.4 Hz, 1H), 8.11 (d, J = 8.4 Hz, 1H), 7.44 (s, 1H), 6.85 (s, 1H), 3.24 (dd, J = 13.6, 9.8 Hz, 2H), 3.05 (q, J = 13.7 Hz, 2H) | Alternate condition (1) was used |
| | 3,3-difluoro-1-(6-(3H-[1,2,3]triazolo[4,5-b]pyridin-6- | | |
| | yl)thieno[2,3-b]pyridin-2-yl)cyclobutanol | | |

### Group 15

Example 15-1: (2R)-2-(3,3-difluorocyclobutyl)-2-(6-(2-methyl-2H-pyrazolo[3,4-b]pyridin-5-yl)thieno[2,3-b]pyridin-2-yl)ethanol B80R; (2S)-2-(3,3-difluorocyclobutyl)-2-(6-(2-methyl-2H-pyrazolo[3,4-b]pyridin-5-yl)thieno[2,3-b]pyridin-2-yl)ethanol B80S

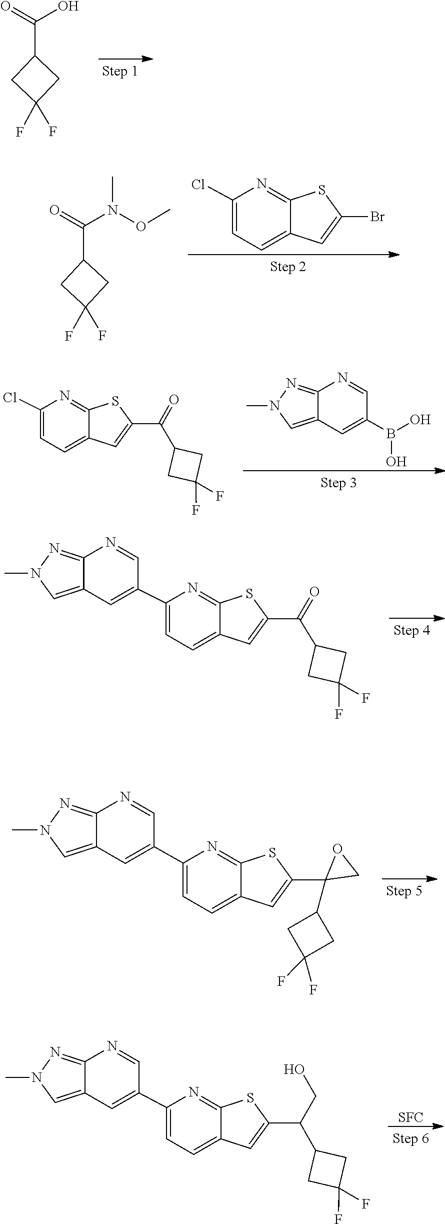

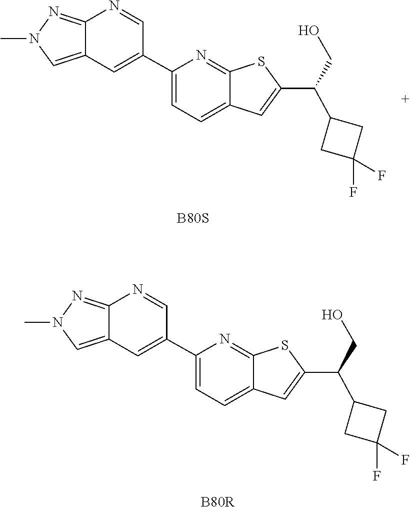

Step 1: 3,3-difluoro-N-methoxy-N-methylcyclobutane-1-carboxamide. To a solution of 3,3-difluorocyclobutane-1-carboxylic acid (50 g, 367 mmol) in THF (1000 mL) was added N,O-dimethyl hydroxylamine hydrochloride salt (108 g, 1.10 mmol), HATU (419 g, 1.10 mol) and diisopropylamine (262 mL, 1.84 mol). The reaction mixture was stirred at rt for 16 h. The reaction mixture was diluted with H₂O (1 L) and extracted with EtOAc (2×1 L). The combined organic extracts were washed with 10% aq. NaHCO₃ (1 L), H₂O (1 L), dried over Na₂SO₄, filtered, and concentrated under reduced pressure to provide a crude material. The crude material was purified by chromatography, eluting with a gradient of 30% to 60% EtOAc in pet. ether, to provide 3,3-difluoro-N-methoxy-N-methylcyclobutane-1-carboxamide (49 g, 74% yield). m/z (ESI): 180.1(M+H)⁺. ¹H NMR (400 MHz, Chloroform-d) δ 3.70 (s, 3H), 3.29 (t, J=9.5 Hz, 1H), 3.23 (s, 3H), 2.96-2.82 (m, 2H), 2.78-2.65 (m, 2H).

Step 2: (6-chlorothieno[2,3-b]pyridin-2-yl)(3,3-difluorocyclobutyl)methanone. To a solution of 2-bromo-6-chlorothieno[2,3-b]pyridine (15 g, 60.4 mmol) in THF (150 mL) at −78° C. under N₂ was added a solution of n-butyllithium in hexanes (2.5 M, 29 mL, 72.4 mmol). The mixture was stirred for 2 min, followed by addition of a solution of 3,3-difluoro-N-methoxy-N-methylcyclobutane-1-carboxamide (14.06 g, 78 mmol) in THF (50 mL). The mixture was stirred for 15 min at −78° C. Then, the reaction was quenched by addition of satd. aq. NH₄Cl (200 mL), extracted with EtOAc (2×250 mL), dried over Na₂SO₄, filtered, and concentrated to give a crude material. The crude material was purified by column chromatography, eluting with a gradient of 10% EtOAc in pet. ether, to provide (6-chlorothieno[2,3-b]pyridin-2-yl)(3,3-difluorocyclobutyl)methanone (11.8 g, 68% yield). m/z (ESI): 288.0 (M+H)⁺. ¹H NMR (400 MHz, DMSO-d₆) δ 8.47 (d, J=8.4 Hz, 1H), 8.42 (s, 1H), 7.65 (d, J=8.5 Hz, 1H), 4.10 (pd, J=8.6, 2.8 Hz, 1H), 2.97 (dqdd, J=16.9, 13.8, 10.1, 7.4 Hz, 4H).

Step 3: (3,3-difluorocyclobutyl)(6-(2-methyl-2H-pyrazolo[3,4-b]pyridin-5-yl)thieno[2,3-b]pyridin-2-yl)methanone. A solution of (6-chlorothieno[2,3-b]pyridin-2-yl)(3,3-difluorocyclobutyl)methanone (1.5 g, 5.21 mmol), K₂CO₃ (1.441 g, 10.43 mmol) and (2-methyl-2H-pyrazolo[3,4-b]pyridin-5-yl)boronic acid (1.199 g, 6.78 mmol) in 1,4-dioxane (15 mL) and H₂O (2 mL) was purged with N₂ for 10 min. Next, PdCl₂(dppf)-DCM adduct (0.426 g, 0.52 mmol) was added, and the reaction mixture was stirred at 90° C. for 2 h. The reaction mixture was concentrated under reduced pressure to give a crude material. The crude material was purified by column chromatography, eluting with 0-3% MeOH in DCM, to give (3,3-difluorocyclobutyl)(6-(2-methyl-2H-pyrazolo[3,4-b]pyridin-5-yl)thieno[2,3-b]pyridin-2-yl)methanone (1.4 g, 3.64 mmol, 70% yield). m/z (ESI): 385.2 (M+H)⁺. ¹H NMR (401 MHz, DMSO-d₆) δ 9.44 (d, J=2.4 Hz, 1H), 9.04 (d, J=2.3 Hz, 1H), 8.60 (s, 1H), 8.52 (d, J=8.5 Hz, 1H), 8.42 (s, 1H), 8.27 (d, J=8.6 Hz, 1H), 4.26 (s, 3H), 4.17-4.09 (m, 1H), 3.05-2.93 (m, 4H).

Step 4: 2-(2-(3,3-difluorocyclobutyl)oxiran-2-yl)-6-(2-methyl-2H-pyrazolo[3,4-b]pyridin-5-yl)thieno[2,3-b]pyridine. To a solution of potassium tert-butoxide (0.490 g, 4.37 mmol) in DMSO (28.0 mL) at rt was added trimethylsulfoxonium iodide (1.042 g, 4.73 mmol), and the mixture was stirred for 30 min. A solution of (3,3-difluorocyclobutyl)(6-(2-methyl-2H-pyrazolo[3,4-b]pyridin-5-yl)thieno[2,3-b]pyridin-2-yl)methanone (1.4 g, 3.64 mmol) in DMSO (28.0 mL) was added, and the mixture was stirred for 48 h. The reaction mixture was combined with another batch of material, and the mixture was diluted with EtOAc (100 mL) and H₂O (150 mL). The aqueous layer was separated and extracted with EtOAc (3×100 mL) and 15% MeOH in DCM (4×150 mL). The organic extracts were combined, dried over Na₂SO₄, and concentrated under reduced pressure to provide a crude material. The crude material was purified by column chromatography, eluting with a mobile phase of 0-5% MeOH in DCM, to give 2-(2-(3,3-difluorocyclobutyl)oxiran-2-yl)-6-(2-methyl-2H-pyrazolo[3,4-b]pyridin-5-yl)thieno[2,3-b]pyridine (1.05 g). m/z (ESI): 399.2 (M+H)⁺. ¹H NMR (401 MHz, DMSO-d₆): δ 9.38 (d, J=2.3 Hz, 1H), 8.90 (d, J=2.3 Hz, 1H), 8.55 (s, 1H), 8.29 (d, J=8.4 Hz, 1H), 8.14 (d, J=8.5 Hz, 1H), 7.60 (s, 1H), 4.25 (s, 3H), 3.42-3.37 (m, 1H), 3.19 (q, J=4.2 Hz, 2H), 2.85 (dtd, J=14.0, 8.9, 4.5 Hz, 1H), 2.73-2.57 (m, 1H), 2.44-2.30 (m, 2H).

Step 5: 2-(3,3-difluorocyclobutyl)-2-(6-(2-methyl-2H-pyrazolo[3,4-b]pyridin-5-yl)thieno[2,3-b]pyridin-2-yl)ethanol. To a stirred solution of 2-(2-(3,3-difluorocyclobutyl)oxiran-2-yl)-6-(2-methyl-2H-pyrazolo[3,4-b]pyridin-5-yl)thieno[2,3-b]pyridine (0.45 g, 1.13 mmol) in MeOH (9.0 mL) and THF (9.0 mL) was added palladium acetate (0.254 g, 1.13 mmol) and 10 wt. % Pd/C (0.12 g, 0.11 mmol). The reaction mixture was stirred at rt for 18 h under an H₂ bladder. Then, the reaction was filtered, washed with 20% MeOH in DCM (3×75 mL) and concentrated under reduced pressure to provide a crude material. The crude material was purified by column chromatography, eluting with a mobile phase of 0-5% MeOH in DCM, to afford 2-(3,3-difluorocyclobutyl)-2-(6-(2-methyl-2H-pyrazolo[3,4-b]pyridin-5-yl)thieno[2,3-b]pyridin-2-yl)ethan-1-ol (0.15 g, 0.38 mmol, 33% yield). m z (ESI): 401.2 (M+H)⁺. ¹H NMR (401 MHz, DMSO-d₆): δ 9.38 (d, J=2.3 Hz, 1H), 8.90 (d, J=2.3 Hz, 1H), 8.54 (s, 1H), 8.24 (d, J=8.4 Hz, 1H), 8.08 (d, J=8.4 Hz, 1H), 7.29 (s, 1H), 4.96 (t, J=5.3 Hz, 1H), 4.24 (s, 3H), 3.69 (t, J=5.2 Hz, 2H), 3.20-3.15 (m, 1H), 2.83-2.71 (m, 2H), 2.60-2.55 (m, 2H), 2.34-2.30 (m, 1H).

Step 6: SFC Purification. The sample was purified by SFC with a Chiralpak IC 150×50 mm, 5 μm column, eluting with 50% (1:1) MeOH:ACN in CO₂ with a flow rate of 190 mL/min to provide peak 1 and peak 2.

Peak 1: B80S (0.135 g, 0.34 mmol, 34% yield). m/z (ESI): 401.2 (M+H)⁺. ¹H NMR (401 MHz, Methanol-d₄) δ 9.37 (d, J=2.3 Hz, 1H), 8.88 (d, J=2.3 Hz, 1H), 8.43 (s, 1H), 8.21 (d, J=8.4 Hz, 1H), 7.97 (d, J=8.4 Hz, 1H), 7.25 (s, 1H), 4.31 (s, 3H), 3.88-3.82 (m, 2H), 3.22-3.13 (m, 1H), 2.88-2.78 (m, 1H), 2.58-2.55 (m, 3H), 2.41-2.27 (m, 1H).

Peak 2: B80R (0.098 g, 0.25 mmol, 25% yield). m/z (ESI): 401.2 (M+H)⁺. ¹H NMR (401 MHz, Methanol-d₄) δ 9.37 (d, J=2.3 Hz, 1H), 8.88 (d, J=2.3 Hz, 1H), 8.43 (s, 1H), 8.21 (d, J=8.4 Hz, 1H), 7.97 (d, J=8.4 Hz, 1H), 7.25 (s, 1H), 4.31 (s, 3H), 3.88-3.82 (m, 2H), 3.22-3.13 (m, 1H), 2.88-2.78 (m, 1H), 2.58-2.55 (m, 3H), 2.41-2.27 (m, 1H).

Example 15-2:3,3-difluoro-1-(6-(2-methyl-2H-pyrazolo[3,4-b]pyridin-5-yl)-1-benzothiophen-2-yl)cyclobutanol C48

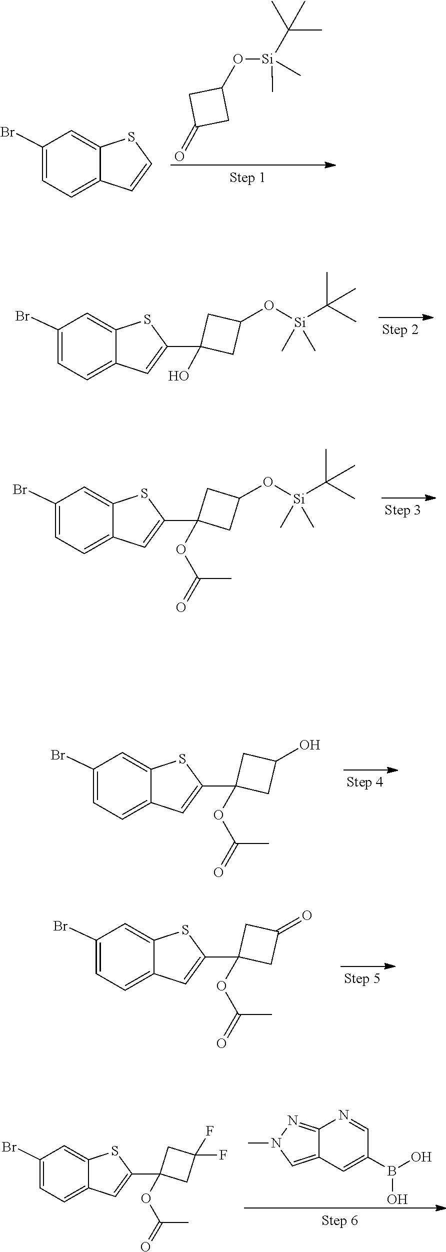

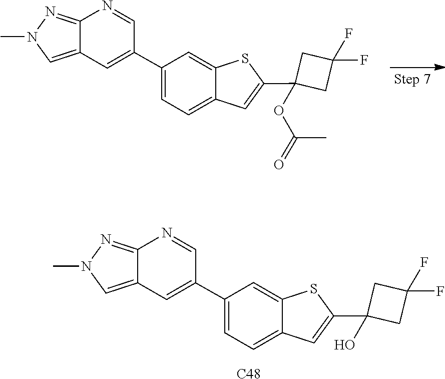 [SMILES: CC(=O)OC1(c2cc3ccc(-c4cnc5nn(C)cc5c4)cc3s2)CC(F)(F)C1 ; Cn1cc2cc(-c3ccc4cc(C5(O)CC(F)(F)C5)sc4c3)cnc2n1 ; "C48"]

Step 1: 1-(6-bromobenzo[b]thiophen-2-yl)-3-((tert-butyldimethylsilyl)oxy)cyclobutan-1-ol. To a solution of 6-bromobenzo[b]thiophene (550 mg, 2.58 mmol) in THF (15 mL) at −78° C. was added a solution of n-butyllithium in THF (2.0 M, 3226 μL, 6.45 mmol) dropwise over 10 min, and the mixture was stirred at −78° C. for 1 h. Then, 3-((tert-butyldimethylsilyl)oxy)cyclobutan-1-one (931 mg, 4.65 mmol) in THF (5 mL) was added dropwise over 5 min, and the reaction mixture was stirred at −78° C. for 1 h. After, the reaction mixture was diluted with satd. aq. NH₄Cl (25 mL), extracted with EtOAc (2×50 mL), dried over Na₂SO₄, filtered, and concentrated under reduced pressure to give a crude material. The crude material was purified by chromatography, eluting with a gradient of 1% to 20% EtOAc in hexane, to provide 1-(6-bromobenzo[b]thiophen-2-yl)-3-((tert-butyldimethylsilyl)oxy)cyclobutan-1-ol (500 mg, 1.21 mmol, 47% yield). ¹H NMR (401 MHz, Chloroform-d) δ 7.96 (d, J=1.8 Hz, 1H), 7.60 (d, J=8.4 Hz, 1H), 7.47 (dd, J=8.4, 1.8 Hz, 1H), 7.16 (d, J=0.8 Hz, 1H), 4.17 (p, J=6.8 Hz, 1H), 3.06-2.94 (m, 2H), 2.55-2.44 (m, 2H), 0.92 (s, 9H), 0.08 (s, 6H).

Step 2: 1-(6-bromobenzo[b]thiophen-2-yl)-3-((tert-butyldimethylsilyl)oxy)cyclobutyl acetate. To a solution of 1-(6-bromobenzo[b]thiophen-2-yl)-3-((tert-butyldimethylsilyl)oxy)cyclobutan-1-ol (800 mg, 1.94 mmol) in DCM (20 mL) at 0° C. was added acetic anhydride (913 μL, 9.67 mmol), N,N-dimethylpyridin-4-amine (118 mg, 0.97 mmol) and Et₃N (539 μL, 3.87 mmol). The reaction mixture was stirred at rt for 1 h. Then, the reaction mixture was diluted with H₂O (20 mL), extracted with DCM (2×20 mL), washed with brine (50 mL), and dried over Na₂SO₄. Next, the mixture was filtered and concentrated under reduced pressure to give a crude material. The crude material was purified by chromatography, eluting with a gradient of 1% to 20% EtOAc in hexane, to provide 1-(6-bromobenzo[b]thiophen-2-yl)-3-((tert-butyldimethylsilyl)oxy)cyclobutyl acetate (800 mg, 1.76 mmol, 91% yield). ¹H NMR (401 MHz, Chloroform-d) δ 7.92 (d, J=1.8 Hz, 1H), 7.60 (dd, J=8.4, 1.4 Hz, 1H), 7.46 (dd, J=8.5, 1.7 Hz, 1H), 7.25 (d, J=0.9 Hz, 1H), 3.24-3.15 (m, 2H), 3.11 (d, J=1.0 Hz, 1H), 3.07 (q, J=7.3 Hz, 2H), 2.05 (d, J=1.3 Hz, 3H), 0.91 (d, J=1.3 Hz, 9H), 0.07 (d, J=1.4 Hz, 6H).

Step 3: 1-(6-bromobenzo[b]thiophen-2-yl)-3-hydroxycyclobutyl acetate. To a solution of 1-(6-bromobenzo[b]thiophen-2-yl)-3-((tert-butyldimethylsilyl)oxy)cyclobutyl acetate (800 mg, 1.76 mmol) in THF (20 mL) at 0° C. was added TBAF (1.0 M in THF, 5269 μL, 5.27 mmol), and the reaction was stirred for 2 h. After, the reaction was diluted with H₂O (100 mL), extracted with EtOAc (2×100 mL), washed with brine (2×50 mL), and dried over Na₂SO₄. The solution was filtered and concentrated under reduced pressure to give a crude material. The crude material was purified by chromatography, eluting with a gradient of 1% to 20% EtOAc in hexane, to provide 1-(6-bromobenzo[b]thiophen-2-yl)-3-hydroxycyclobutyl acetate (580 mg, 1.70 mmol, 97% yield). ¹H NMR (401 MHz, DMSO-d₆) δ 8.21 (d, J=1.8 Hz, 1H), 7.74 (d, J=8.5 Hz, 1H), 7.53 (dd, J=8.5, 1.9 Hz, 1H), 7.48 (s, 1H), 5.40 (dd, J=6.4, 1.4 Hz, 1H), 4.06 (p, J=7.0 Hz, 1H), 3.05 (ddt, J=9.4, 5.6, 2.8 Hz, 2H), 2.47-2.43 (m, 2H), 2.02 (d, J=1.4 Hz, 3H).

Step 4: 1-(6-bromobenzo[b]thiophen-2-yl)-3-oxocyclobutyl acetate. A mixture of 1-(6-bromobenzo[b]thiophen-2-yl)-3-hydroxycyclobutyl acetate (580 mg, 1.70 mmol) and IBX (1.43 mg, 5.10 mmol) in EtOAc (20 mL) was refluxed at 75° C. for 16 h. The reaction mixture was cooled to rt, filtered, and washed with EtOAc (50 mL). Then, the filtrate was concentrated under reduced pressure to give 1-(6-bromobenzo[b]thiophen-2-yl)-3-oxocyclobutyl acetate (550 mg, 1.62 mmol, 95% yield). ¹H NMR (401 MHz, DMSO-d₆) δ 8.26 (d, J=1.9 Hz, 1H), 7.76 (d, J=8.6 Hz, 1H), 7.60 (s, 1H), 7.55 (dd, J=8.4, 1.9 Hz, 1H), 3.88-3.80 (m, 4H), 2.10 (d, J=1.0 Hz, 3H).

Step 5: 1-(6-bromobenzo[b]thiophen-2-yl)-3,3-difluorocyclobutyl acetate. To a solution of 1-(6-bromobenzo[b]thiophen-2-yl)-3-oxocyclobutyl acetate (20 mg, 0.059 mmol) in DCM (1 mL) at 0° C. was added DAST (23.37 μL, 0.18 mmol) in DCM (1 mL). The reaction mixture was stirred at rt for 16 h. Then, the reaction mixture was diluted with NaHCO₃ (5 mL), extracted with DCM (2×5 mL), washed with brine (5 mL), and dried over Na₂SO₄. The mixture was filtered and concentrated under reduced pressure to give crude 1-(6-bromobenzo[b]thiophen-2-yl)-3,3-difluorocyclobutyl acetate, which was used directly for the next step without further purification. ¹H NMR (401 MHz, DMSO-d₆) δ 8.26 (d, J=1.8 Hz, 1H), 7.78 (dd, J=8.6, 1.6 Hz, 1H), 7.60 (d, J=1.6 Hz, 1H), 7.56 (dt, J=8.5, 1.7 Hz, 1H), 3.52-3.37 (m, 5H), 2.07 (d, J=1.5 Hz, 3H).

Step 6: 3,3-difluoro-1-(6-(2-methyl-2H-pyrazolo[3,4-b]pyridin-5-yl)benzo[b]thiophen-2-yl)cyclobutyl acetate. A solution of 1-(6-bromobenzo[b]thiophen-2-yl)-3,3-difluorocyclobutyl acetate (150 mg, 0.42 mmol), (2-methyl-2H-pyrazolo[3,4-b]pyridin-5-yl)boronic acid (88 mg, 0.50 mmol) and K₂CO₃ (172 mg, 1.25 mmol) in 1,4-dioxane (2 mL) and H₂O (0.4 mL) was purged for 5 min under N₂ gas. Then, SPhos Pd G3 (32.4 mg, 0.042 mmol) was added, and the reaction mixture was stirred at 80° C. for 1 h. After, the reaction mixture was cooled to rt, diluted with H₂O (5 mL), and extracted with EtOAc (2×20 mL). The combined organic extracts were washed with brine (25 mL), dried over Na₂SO₄, filtered and concentrated under reduced pressure to give crude 3,3-difluoro-1-(6-(2-methyl-2H-pyrazolo[3,4-b]pyridin-5-yl)benzo[b]thiophen-2-yl)cyclobutyl acetate (220 mg), which was used directly in the next step without further purification. m/z (ESI): 414.2 (M+H)⁺

Step 7: 3,3-difluoro-1-(6-(2-methyl-2H-pyrazolo[3,4-b]pyridin-5-yl)-1-benzothiophen-2-yl)cyclobutanol. A mixture of 3,3-difluoro-1-(6-(2-methyl-2H-pyrazolo[3,4-b]pyridin-5-yl)benzo[b]thiophen-2-yl)cyclobutyl acetate (300 mg, 0.73 mmol) and K₂CO₃ (301 mg, 2.18 mmol) in MeOH (10 mL) was stirred at rt for 1 h. The mixture was concentrated under reduced pressure, taken up in EtOAc (25 mL), filtered and concentrated under reduced pressure to provide a crude material. The crude material was purified by preparative HPLC, using a mobile phase of 5-95% (0.1% formic acid) H₂O in ACN with a flow rate of 2.0 mL/min, to provide Compound C48 (70 mg, 0.19 mmol, 26% yield). m z (ESI): 372.2 (M+H)⁺. ¹H NMR (401 MHz, DMSO-d₆) δ 9.00 (d, J=2.4 Hz, 1H), 8.51 (d, J=2.5 Hz, 1H), 8.49 (s, 1H), 8.35 (d, J=1.8 Hz, 1H), 7.91 (d, J=8.3 Hz, 1H), 7.76 (dd, J=8.4, 1.8 Hz, 1H), 7.47 (s, 1H), 6.74 (d, J=1.6 Hz, 1H), 4.24 (s, 3H), 3.22 (qd, J=11.6, 9.6, 6.6 Hz, 2H), 3.04 (td, J=14.2, 11.7 Hz, 2H).

### Group 16

Example 16-1: (S)-(3,3-difluorocyclobutyl)(6-(3-methyl-3H-[1,2,3]triazolo[4,5-b]pyridin-6-yl)thieno[2,3-b]pyridin-2-yl)methanol B82S

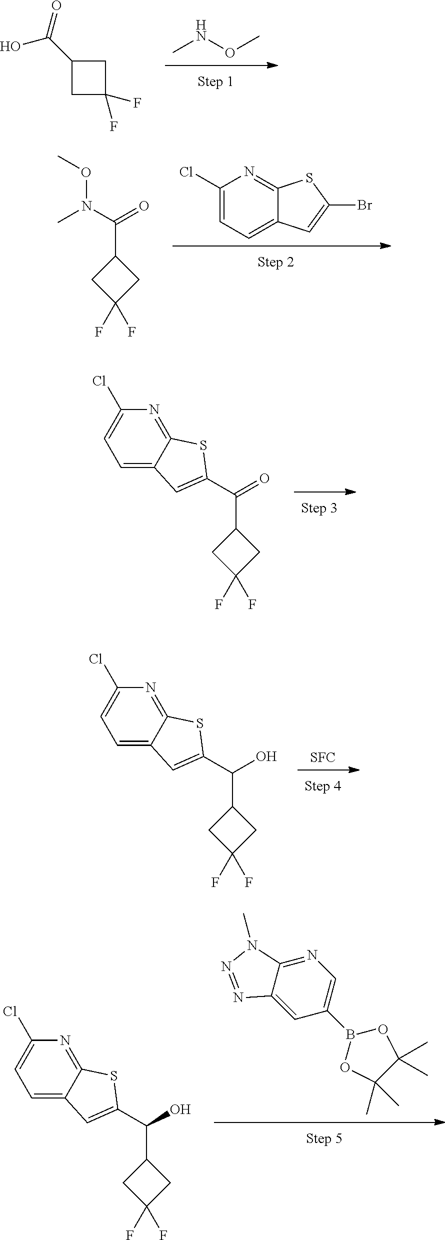

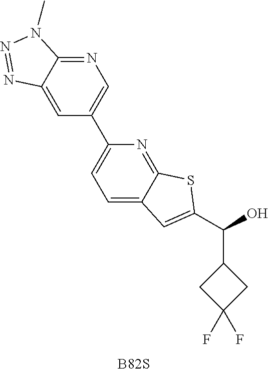 [SMILES: "B82S" ; Cn1nnc2cc(-c3ccc4cc([C@@H](O)C5CC(F)(F)C5)sc4n3)cnc21]

Step 1: 3,3-difluoro-N-methoxy-N-methylcyclobutane-1-carboxamide. To a solution of 3,3-difluorocyclobutane-1-carboxylic acid (50 g, 367 mmol) in THF (1000 mL) was added N,O-dimethylhydroxylamine hydrochloride (108 g, 1.10 mol), HATU (419 g, 1.10 mol) and diisopropylamine (262 mL, 1.84 mol). The reaction mixture was stirred at rt for 18 h. The reaction mixture was diluted with H₂O (1 L), extracted with EtOAc (2×1 L), washed with 10% aq. NaHCO₃ (1 L), H₂O (1 L), and dried over Na₂SO₄. The solution was filtered and concentrated under reduced pressure to give a crude material. The crude material was purified by chromatography, eluting with a gradient of 30% to 60% EtOAc/pet. ether, to provide 3,3-difluoro-N-methoxy-N-methylcyclobutane-1-carboxamide (49 g, 74% yield). m z (ESI): 180.1 (M+H)⁺. ¹H NMR (400 MHz, Chloroform-d) δ 3.70 (s, 3H), 3.29 (t, J=9.5 Hz, 1H), 3.23 (s, 3H), 2.96-2.82 (m, 2H), 2.78-2.65 (m, 2H).

Step 2: (6-chlorothieno[2,3-b]pyridin-2-yl)(3,3-difluorocyclobutyl)methanone. To a solution of 2-bromo-6-chlorothieno[2,3-b]pyridine (45 g, 181 mmol) in THF (450 mL) at −78° C. under N₂ was added n-butyllithium in hexane (2.5 M, 87 mL, 217 mmol), and the mixture was stirred for 2 min. Then, a solution of 3,3-difluoro-N-methoxy-N-methylcyclobutane-1-carboxamide (42.2 g, 235 mmol) in THF (100 mL) was added, and the reaction was stirred for 15 min at −78° C. The reaction mass was quenched by addition of satd. aq. NH₄Cl (500 mL), extracted with EtOAc (2×1 L), dried over Na₂SO₄, filtered, and concentrated under reduced pressure to provide a crude material. The crude material was purified by column chromatography, eluting with a gradient of 10% EtOAc/pet. ether, to provide (6-chlorothieno[2,3-b]pyridin-2-yl)(3,3-difluorocyclobutyl)methanone (36 g, 69% yield). m/z (ESI): 288.0 (M+H)⁺. ¹H NMR (400 MHz, DMSO-d₆) δ 8.47 (d, J=8.4 Hz, 1H), 8.42 (s, 1H), 7.65 (d, J=8.5 Hz, 1H), 4.10 (pd, J=8.6, 2.8 Hz, 1H), 2.97 (dqdd, J=16.9, 13.8, 10.1, 7.4 Hz, 4H).

Step 3: (6-chlorothieno[2,3-b]pyridin-2-yl)(3,3-difluorocyclobutyl)methanol. A solution of (6-chlorothieno[2,3-b]pyridin-2-yl)(3,3-difluorocyclobutyl)methanone (36 g, 125 mmol) in MeOH (720 mL) was cooled to 0° C., and NaBH₄ (9.47 g, 250 mmol) was added. Then, the reaction was stirred at rt for 1 h. After, the reaction mixture was quenched by addition of H₂O (720 mL), concentrated under reduced pressure, diluted with H₂O (500 mL) and extracted with EtOAc (2×1 L). The organic extracts were washed with brine (500 mL), dried over Na₂SO₄, filtered, and concentrated under reduced pressure to provide (6-chlorothieno[2,3-b]pyridin-2-yl)(3,3-difluorocyclobutyl)methanol (35 g, 97% yield). m z (ESI): 290.0 (M+H)⁺. ¹H NMR (400 MHz, DMSO-d₆) δ 8.22 (d, J=8.4 Hz, 1H), 7.49 (d, J=8.3 Hz, 1H), 7.34 (s, 1H), 6.30 (d, J=5.2 Hz, 1H), 4.92 (t, J=5.1 Hz, 1H), 2.65-2.53 (m, 4H).

Step 4: SFC Purification. The sample was purified via SFC using a Chiral Pak AS-H 250×30 mm, 5 μm column with a mobile phase of 10% (0.2% DEA) MeOH in CO₂ with a flow rate of 150 mL/min to generate peak 1 and peak 2.

Peak-1: ¹H NMR (401 MHz, DMSO-d₆) δ 8.23 (d, J=8.3 Hz, 1H), 7.49 (d, J=8.4 Hz, 1H), 7.34 (s, 1H), 6.31 (d, J=5.2 Hz, 1H), 4.92 (t, J=5.0 Hz, 1H), 2.68-2.51 (m, 4H).

Peak-2: ¹H NMR (401 MHz, DMSO-d₆) δ 8.22 (d, J=8.4 Hz, 1H), 7.49 (d, J=8.4 Hz, 1H), 7.34 (s, 1H), 6.31 (d, J=5.2 Hz, 1H), 4.92 (t, J=5.1 Hz, 1H), 2.69-2.54 (m, 1H), 2.54 (d, J=7.3 Hz, 2H).

Step 5: (S)-(3,3-difluorocyclobutyl)(6-(1-methyl-1H-[1,2,3]triazolo[4,5-b]pyridin-6-yl)thieno[2,3-b]pyridin-2-yl)methanol. To a solution of (S)-(6-chlorothieno[2,3-b]pyridin-2-yl)(3,3-difluorocyclobutyl)methanol (Peak 1 from SFC Purification, 200 mg, 0.69 mmol) in 1,4-dioxane (4.00 mL) and H₂O (0.40 mL) was added 3-methyl-6-(4,4,5,5-tetramethyl-1,3,2-dioxaborolan-2-yl)-3H-[1,2,3]triazolo[4,5-b]pyridine (233 mg, 0.90 mmol) and K₂CO₃ (286 mg, 2.07 mmol), and the mixture was degassed with N₂ for 5 min. Then, Pd(dppf)Cl₂ (50.5 mg, 0.069 mmol) was added, and the reaction mixture was stirred at 105° C. for 2 h. The reaction mass was concentrated, diluted with EtOAc (10 mL), and washed with H₂O (10 mL). The organic layer was washed with brine (5 mL), dried over Na₂SO₄, filtered, and concentrated to afford a crude material. The crude material was purified by preparative HPLC, eluting with a gradient of 5-95% (0.1% formic acid) H₂O in ACN with a flow rate of 2.0 mL/min, to afford Compound B82S (70 mg, 0.18 mmol, 26% yield). m/z (ESI): 388.2 (M+H)⁺. ¹H NMR (400 MHz, DMSO-d₆) δ 9.57 (d, J=2.0 Hz, 1H), 9.21 (d, J=2.1 Hz, 1H), 8.33 (d, J=8.4 Hz, 1H), 8.23 (d, J=8.4 Hz, 1H), 7.38 (s, 1H), 6.29 (d, J=4.7 Hz, 1H), 4.95 (d, J=5.0 Hz, 1H), 4.37 (s, 3H), 2.72-2.54 (m, 5H).

Example 16-2: (R)-(3,3-difluorocyclobutyl)(6-(3-methyl-3H-[1,2,3]triazolo[4,5-b]pyridin-6-yl)thieno[2,3-b]pyridin-2-yl)methanol B82R

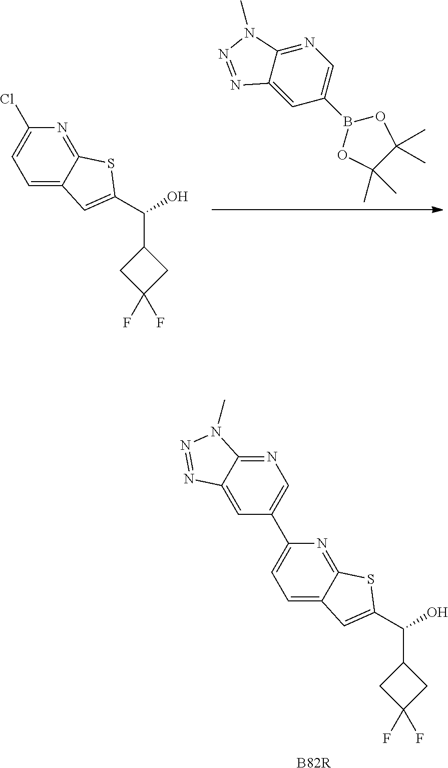 [SMILES: O[C@@H](c1cc2ccc(Cl)nc2s1)C1CC(F)(F)C1 ; Cn1nnc2cc(B3OC(C)(C)C(C)(C)O3)cnc21 ; "B82R" ; Cn1nnc2cc(-c3ccc4cc([C@H](O)C5CC(F)(F)C5)sc4n3)cnc21]

(R)-(3,3-difluorocyclobutyl)(6-(1-methyl-1H-[1,2,3]triazolo[4,5-b]pyridin-6-yl)thieno[2,3-b]pyridin-2-yl)methanol. To a solution of (R)-(6-chlorothieno[2,3-b]pyridin-2-yl)(3,3-difluorocyclobutyl)methanol (Peak 2 from SFC purification, 300 mg, 1.04 mmol) in 1,4-dioxane (10 mL) and H₂O (1 mL) was added 3-methyl-6-(4,4,5,5-tetramethyl-1,3,2-dioxaborolan-2-yl)-3H-[1,2,3]triazolo[4,5-b]pyridine (546 mg, 1.55 mmol) and K₂CO₃ (429 mg, 3.11 mmol), and the mixture was degassed with N₂ for 5 min. Then, Pd(dppf)Cl₂ (76 mg, 0.10 mmol) was added, and the reaction mixture was stirred at 105° C. for 2 h. The reaction mass was concentrated, diluted with EtOAc (50 mL), washed with H₂O (50 mL) and separated. The organic layer was washed with brine (15 mL), dried over Na₂SO₄, filtered, and concentrated to afford a crude material. The crude material was purified by prep HPLC, eluting with a gradient of 5-95% (0.1% formic acid) H₂O in ACN with a flow rate of 2.0 mL/min, to afford Compound B82R (52 mg, 0.13 mmol, 13% yield). m/z (ESI): 388.2 (M+H)⁺. ¹H NMR (400 MHz, DMSO-d₆) δ 9.58 (d, J=1.9 Hz, 1H), 9.22 (d, J=2.0 Hz, 1H), 8.33 (d, J=8.4 Hz, 1H), 8.24 (d, J=8.4 Hz, 1H), 7.38 (s, 1H), 6.33 (s, 1H), 4.96 (d, J=4.4 Hz, 1H), 4.37 (s, 3H), 2.69-2.53 (m, 5H).

Example 16-3:1-(6-(1,2-benzoxazol-5-yl)thieno[2,3-b]pyridin-2-yl)-3,3-difluorocyclobutanol C50

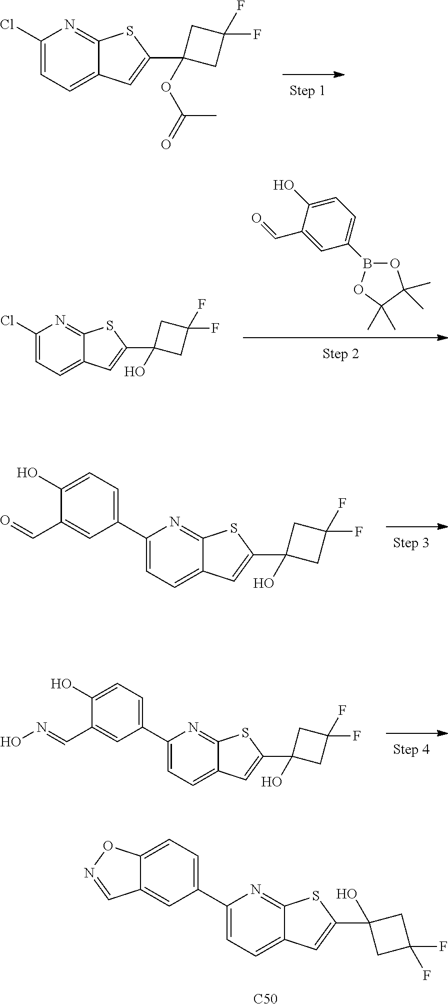

Step 1: 1-(6-chlorothieno[2,3-b]pyridin-2-yl)-3,3-difluorocyclobutan-1-ol. To a solution of 1-(6-chlorothieno[2,3-b]pyridin-2-yl)-3,3-difluorocyclobutyl acetate (0.2 g, 0.63 mmol) in MeOH (6.00 mL) and H₂O (0.60 mL) was added K₂CO₃ (0.261 g, 1.89 mmol) at rt, and the mixture was stirred for 1 h. Then, the solution was concentrated under reduced pressure, diluted with H₂O (20 mL), extracted with EtOAc (20 mL×3), washed with brine (20 mL), dried over Na₂SO₄, and concentrated under reduced pressure to afford crude 1-(6-chlorothieno[2,3-b]pyridin-2-yl)-3,3-difluorocyclobutan-1-ol (0.14 g, 0.508 mmol, 81% yield), which was used in the next step without further purification. m/z (ESI): 276.0 (M+H)⁺. H NMR (400 MHz, DMSO-d₆) δ 8.26 (d, J=8.4 Hz, 1H), 7.52 (d, J=8.4 Hz, 1H), 7.48 (s, 1H), 6.91 (s, 1H), 3.29-3.17 (m, 2H), 3.04 (td, J=14.2, 12.0 Hz, 2H).

Step 2: 5-(2-(3,3-difluoro-1-hydroxycyclobutyl)thieno[2,3-b]pyridin-6-yl)-2-hydroxybenzaldehyde. A stirred solution of 1-(6-chlorothieno[2,3-b]pyridin-2-yl)-3,3-difluorocyclobutan-1-ol (0.1 g, 0.36 mmol), 2-hydroxy-5-(4,4,5,5-tetramethyl-1,3,2-dioxaborolan-2-yl)benzaldehyde (0.135 g, 0.54 mmol) and K₂CO₃ (0.150 g, 1.09 mmol) in 1,4-dioxane (7.20 mL) and H₂O (0.80 mL) was purged with N₂ gas for 5 min. After purging, SPhos Pd G3 (0.028 g, 0.036 mmol) was added, and the reaction mixture was stirred at 110° C. for 3 h. Then, the mixture was diluted with EtOAc, filtered, washed with H₂O (2×25 mL) and dried over Na₂SO₄. The mixture was filtered and concentrated under reduced pressure to give a crude material. The crude material was purified by chromatography, eluting with a gradient of 25% to 60% EtOAc in hexane, to provide 5-(2-(3,3-difluoro-1-hydroxycyclobutyl)thieno[2,3-b]pyridin-6-yl)-2-hydroxybenzaldehyde (120 mg, 65% yield). m/z (ESI): 362.0 (M+H)⁺. ¹H NMR (400 MHz, DMSO-d₆) δ 11.06 (s, 1H), 10.37 (s, 1H), 8.47 (d, J=2.5 Hz, 1H), 8.34 (dd, J=8.7, 2.5 Hz, 1H), 8.24 (d, J=8.5 Hz, 1H), 7.98 (d, J=8.4 Hz, 1H), 7.44 (s, 1H), 7.15 (d, J=8.7 Hz, 1H), 6.82 (s, 1H), 3.27-3.17 (m, 2H), 3.04 (td, J=14.2, 11.8 Hz, 2H).

Step 3: (E)-5-(2-(3,3-difluoro-1-hydroxycyclobutyl)thieno[2,3-b]pyridin-6-yl)-2-hydroxybenzaldehyde oxime. To a stirred solution of 5-(2-(3,3-difluoro-1-hydroxycyclobutyl)thieno[2,3-b]pyridin-6-yl)-2-hydroxybenzaldehyde (0.12 g, 0.33 mmol) in EtOH (4.80 mL) was added sodium acetate (0.054 g, 0.66 mmol) and hydroxylamine hydrochloride (0.030 g, 0.43 mmol) at rt, and the reaction mixture was stirred at 50° C. for 2 h. Then, the solution was concentrated under reduced pressure, diluted with EtOAc, washed with H₂O, dried over Na₂SO₄, filtered, and concentrated under reduced pressure to provide crude (E)-5-(2-(3,3-difluoro-1-hydroxycyclobutyl)thieno[2,3-b]pyridin-6-yl)-2-hydroxybenzaldehyde oxime (0.1 g, 0.27 mmol, 80% yield), which was used directly in the next step without further purification. m/z (ESI): 377.2 (M+H)⁺. ¹H NMR (400 MHz, DMSO-d₆) δ 11.41 (s, 1H), 10.45 (s, 1H), 8.44 (s, 1H), 8.35 (d, J=2.4 Hz, 1H), 8.22 (d, J=8.5 Hz, 1H), 8.04 (dd, J=8.6, 2.4 Hz, 1H), 7.91 (d, J=8.5 Hz, 1H), 7.42 (s, 1H), 7.03 (d, J=8.6 Hz, 1H), 6.80 (s, 1H), 3.27-3.09 (m, 2H), 3.04 (q, J=13.7 Hz, 2H).

Step 4: 1-(6-(benzo[d]isoxazol-5-yl)thieno[2,3-b]pyridin-2-yl)-3,3-difluorocyclobutan-1-ol. A solution oftriphenylphosphine (139 mg, 0.53 mmol) and 2,3-dichloro-5,6-dicyano-1,4-benzoquinone (121 mg, 0.53 mmol) in DCM (4.00 mL) was stirred at rt for 5 min before (E)-5-(2-(3,3-difluoro-1-hydroxycyclobutyl)thieno[2,3-b]pyridin-6-yl)-2-hydroxybenzaldehyde oxime (80 mg, 0.21 mmol) in DCM (2 mL) was added, and the reaction mixture was stirred at rt for 30 min. Then, the solution was concentrated and purified by chromatography, eluting with a gradient of 15% to 40% EtOAc in hexane, to provide Compound C50 (69 mg, 72% yield). m/z (ESI): 359.0 (M+H)⁺. ¹H NMR (400 MHz,) δ 9.34 (d, J=1.1 Hz, 1H), 8.69 (d, J=1.8 Hz, 1H), 8.49 (dd, J=8.9, 1.8 Hz, 1H), 8.31 (d, J=8.4 Hz, 1H), 8.12 (d, J=8.4 Hz, 1H), 7.91 (d, J=8.8 Hz, 1H), 7.48 (s, 1H), 6.87 (s, 1H), 3.32-3.19 (m, 2H), 3.12-3.00 (m, 2H).

Alternate conditions in reference to Example 16-1:
(1) Step 5: SPhos Pd G3 was used as the catalyst instead of Pd(dppf)Cl₂.

The compounds in Table 16-1 was prepared following the procedures described in Group 16, using appropriate starting materials (e.g., aldehyde/ketones and boronate esters). All starting materials are commercially available or are described in the Intermediates section above.

TABLE 16-1

| Ex. # | Chemical Structure & Name | LCMS: (ESI + ve ion) m/z; NMR | Reaction Comments |
|---|---|---|---|
| B86S | 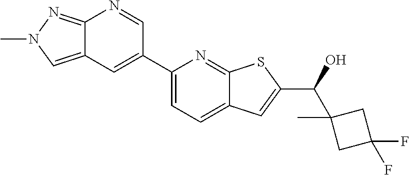 [SMILES: Cn1cc2cc(-c3ccc4cc([C@@H](O)C5(C)CC(F)(F)C5)sc4n3)cnc2n1] | 401.2; ¹H NMR (400 MHz, DMSO-d₆) δ 9.38 (d, J = 2.3 Hz, 1H), 8.92 (d, J = 2.4 Hz, 1H), 8.55 (s, 1H), 8.28 (d, J = 8.4 Hz, 1H), 8.10 (d, J = 8.5 Hz, 1H), 7.35 (s, 1H), 6.41 (s, 1H), 4.88 (s, 1H), 4.24 (s, 3H), 2.94 (p, J = 14.4 Hz, 2H), 2.23 (q, J = 15.2 Hz, 2H), 1.13 (s, 3H) | Alternate condition (1) was used; Before Step 5, the sample was purified via SFC; Peak 1/SFC: Chiralpak AD-H 150 x 50 mm 5 μm column; Mobile phase: 40% (1:1) ACN:MeOH in |
| | (S)-(3,3-difluoro-1-methylcyclobutyl)(6-(2-methyl- | | CO₂ using a flow rate |
| | 2H-pyrazolo[3,4-b]pyridin-5-yl)thieno[2,3-b]pyridin-2- | | of 180 mL/min |
| | yl)methanol | | |
| B86R | 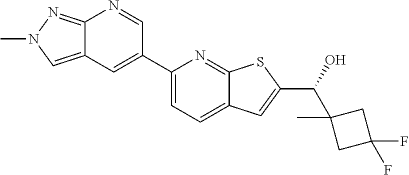 [SMILES: Cn1cc2cc(-c3ccc4cc([C@H](O)C5(C)CC(F)(F)C5)sc4n3)cnc2n1] | 401.2; ¹H NMR (401 MHz, DMSO-d₆) δ 9.38 (d, J = 2.3 Hz, 1H), 8.92 (d, J = 2.3 Hz, 1H), 8.55 (s, 1H), 8.28 (d, J = 8.4 Hz, 1H), 8.10 (d, J = 8.4 Hz, 1H), 7.35 (s, 1H), 6.41 (d, J = 4.4 Hz, 1H), 4.88 (d, J = 4.4 Hz, 1H), 4.24 (s, 3H), 2.94 (p, J = 14.6 Hz, 2H), 2.23 (dd, J = 15.5, 14.6 Hz, 2H), 1.13 (s, 3H) | Alternate condition (1) was used; Before Step 5, the sample was purified via SFC; Peak 2/SFC: Chiralpak AD-H 150 x 50 mm 5 μm column; Mobile phase: 40% (1:1) ACN:MeOH in |
| | (R)-(3,3-difluoro-1-methylcyclobutyl)(6-(2-methyl- | | CO₂ using a flow rate |
| | 2H-pyrazolo[3,4-b]pyridin-5-yl)thieno[2,3-b]pyridin-2- | | of 180 mL/min |
| | yl)methanol | | |
| B87R | 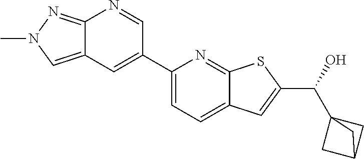 [SMILES: Cn1cc2cc(-c3ccc4cc([C@H](O)C56CC(C5)C6)sc4n3)cnc2n1] | 363.2; ¹H NMR (401 MHz, DMSO-d₆) δ 9.38 (d, J = 2.3 Hz, 1H), 8.91 (d, J = 2.3 Hz, 1H), 8.54 (s, 1H), 8.26 (d, J = 8.4 Hz, 1H), 8.08 (d, J = 8.4 Hz, 1H), 7.22 (d, J = 0.9 Hz, 1H), 5.89 (d, J = 4.4 Hz, 1H), 4.88 (d, J = 4.5 Hz, 1H), 4.24 (s, 3H), 2.58-2.52 (m, 1H), 1.71 (qd, J = 9.5, 1.6 Hz, | Alternate condition (1) was used; Before Step 5, the sample was purified via SFC; Peak 1/SFC: LUX-C4 250 x 50 mm 5 μm column; Mobile phase: 25% of |
| | (R)-bicyclo[1.1.1]pentan-1-yl(6-(2-methyl-2H- | 6H) | (1:1) ACN:MeOH in |
| | pyrazolo[3,4-b]pyridin-5-yl)thieno[2,3-b]pyridin-2- | | CO₂ using a flow rate |
| | yl)methanol | | of 180 mL/min |
| B87S | 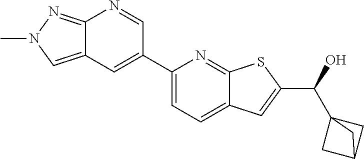 [SMILES: Cn1cc2cc(-c3ccc4cc([C@@H](O)C56CC(C5)C6)sc4n3)cnc2n1] | 363.2; ¹H NMR (401 MHz, DMSO-d₆) δ 9.38 (d, J = 2.3 Hz, 1H), 8.91 (d, J = 2.4 Hz, 1H), 8.55 (s, 1H), 8.26 (d, J = 8.4 Hz, 1H), 8.08 (d, J = 8.4 Hz, 1H), 7.22 (d, J = 0.9 Hz, 1H), 5.90 (s, 1H), 4.88 (s, 1H), 4.24 (s, 3H), 2.48-2.46 (m, 1H), 1.77-1.68 (m, 6H) | Alternate condition (1) was used; Before Step 5, the sample was purified via SFC; Peak 2/SFC: LUX-C4 250 x 50 mm 5 μm column; Mobile phase: 25% of |
| | (S)-bicyclo[1.1.1]pentan-1-yl(6-(2-methyl-2H-pyrazolo[3,4- | | (1:1) ACN:MeOH in |
| | b]pyridin-5-yl)thieno[2,3-b]pyridin-2-yl)methanol | | CO₂ using a flow rate |
| | | | of 180 mL/min |
| B91R | 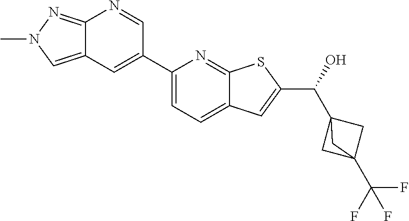 [SMILES: Cn1cc2cc(-c3ccc4cc([C@H](O)C56CC(C(F)(F)F)(C5)C6)sc4n3)cnc2n1] | 431.0; ¹H NMR (400 MHz, DMSO-d₆) δ 9.38 (d, J = 2.3 Hz, 1H), 8.92 (d, J = 2.3 Hz, 1H), 8.55 (s, 1H), 8.28 (d, J = 8.4 Hz, 1H), 8.10 (d, J = 8.4 Hz, 1H), 7.28 (d, J = 1.0 Hz, 1H), 6.15 (d, J = 4.6 Hz, 1H), 5.06 (d, J = 4.5 Hz, 1H), 4.25 (s, 3H), 1.94-1.86 (m, 6H) | Alternate condition (1) was used; Before Step 5, the sample was purified via SFC; Peak 2/SFC: Chiralpak IG 250 x 21 mm, 5 μm column; Mobile phase: 40% (1:1) ACN:MeOH in CO₂ with a flow rate of 100 mL/min |
| | (R)-(6-(2-methyl-2H-pyrazolo[3,4-b]pyridin-5- | | |
| | yl)thieno[2,3-b]pyridin-2-yl)(3-(trifluoromethyl) | | |
| | bicyclo[1.1.1]pentan-1-yl)methanol | | |
| B91S | 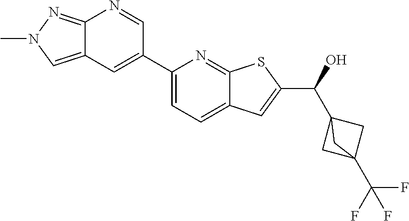 [SMILES: Cn1cc2cc(-c3ccc4cc([C@@H](O)C56CC(C(F)(F)F)(C5)C6)sc4n3)cnc2n1] | 431.1; ¹H NMR (400 MHz, DMSO-d₆) δ 9.38 (d, J = 2.3 Hz, 1H), 8.92 (d, J = 2.3 Hz, 1H), 8.55 (s, 1H), 8.28 (d, J = 8.4 Hz, 1H), 8.10 (d, J = 8.4 Hz, 1H), 7.28 (d, J = 0.9 Hz, 1H), 6.15 (s, 1H), 5.06 (s, 1H), 4.25 (s, 3H), 1.91 (qd, J = 9.4, 1.6 Hz, 6H) | Alternate condition (1) was used; Before Step 5, the sample was purified via SFC; Peak 1/SFC: Chiralpak IG 250 x 21 mm, 5 μm column; Mobile phase: 40% (1:1) ACN:MeOH in CO₂ with a flow rate of 100 mL/min |
| | (S)-(6-(2-methyl-2H-pyrazolo[3,4-b]pyridin-5- | | |
| | yl)thieno[2,3-b]pyridin-2-yl)(3-(trifluoromethyl)bicyclo[1.1.1] | | |
| | pentan-1-yl)methanol | | |

### Group 17

Example 17-1: (R)-(3,3-difluorocyclobutyl)(3-methyl-6-(2-methyl-2H-pyrazolo[3,4-b]pyridin-5-yl)thieno[2,3-b]pyridin-2-yl)methanol B83R; (S)-(3,3-difluorocyclobutyl)(3-methyl-6-(2-methyl-2H-pyrazolo[3,4-b]pyridin-5-yl)thieno[2,3-b]pyridin-2-yl)methanol B83S

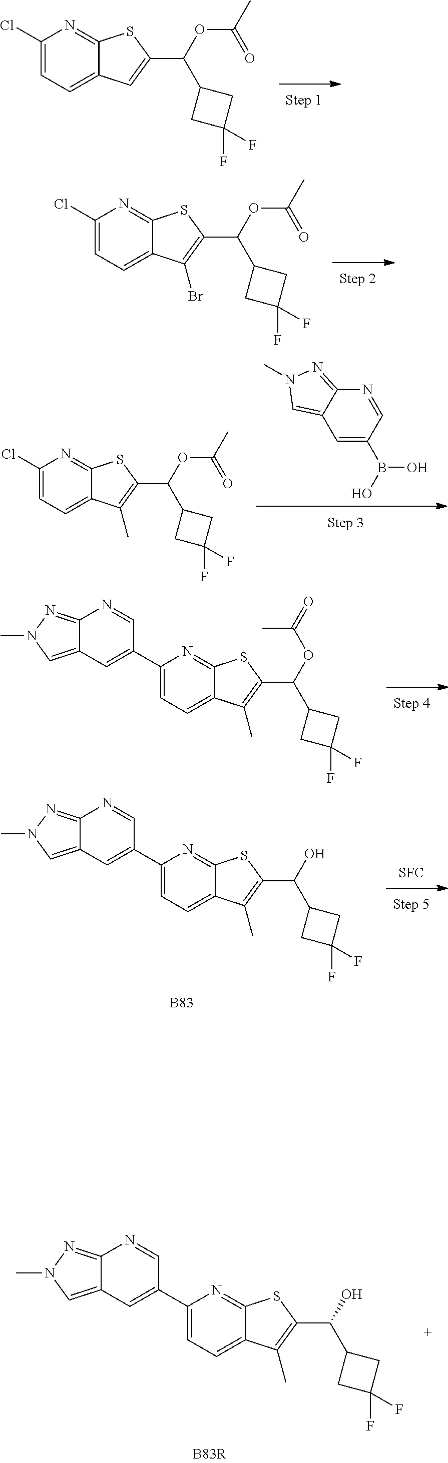

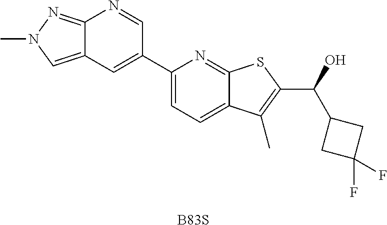 [SMILES: "B83S" ; Cc1c([C@@H](O)C2CC(F)(F)C2)sc2nc(-c3cnc4nn(C)cc4c3)ccc12]

Step 1: (3-bromo-6-chlorothieno[2,3-b]pyridin-2-yl)(3,3-difluorocyclobutyl)methyl acetate. A solution of (6-chlorothieno[2,3-b]pyridin-2-yl)(3,3-difluorocyclobutyl)methyl acetate (2.5 g, 7.54 mmol) and N-bromosuccinimide (26.8 g, 151 mmol) in DMF (50 mL) was sealed and stirred at 70° C. for 24 h. The reaction mixture was cooled to rt. The reaction mixture was diluted with H₂O (250 mL), extracted with EtOAc (2×200 mL), washed with brine (2×200 mL), dried over Na₂SO₄, filtered and concentrated under reduced pressure. The crude material was purified by chromatography, eluting with a gradient of 1%-15% EtOAc in hexane, to provide (3-bromo-6-chlorothieno[2,3-b]pyridin-2-yl)(3,3-difluorocyclobutyl)methyl acetate (2.5 g, 6.09 mmol, 81% yield). m/z (ESI): 410.0 (M+H)⁺. ¹H NMR (401 MHz, DMSO-d₆) δ 8.29 (d, J=8.4 Hz, 1H), 7.55 (d, J=8.0 Hz, 2H), 6.12 (d, J=7.1 Hz, 1H), 2.48-2.40 (m, 1H), 2.85-2.56 (m, 4H), 2.12 (s, 3H).

Step 2: (6-chloro-3-methylthieno[2,3-b]pyridin-2-yl)(3,3-difluorocyclobutyl)methyl acetate. To a solution of (3-bromo-6-chlorothieno[2,3-b]pyridin-2-yl)(3,3-difluorocyclobutyl)methyl acetate (1 g, 2.44 mmol) in 1,4-dioxane (20.0 mL) and H₂O (2.00 mL) was added 2,4,6-trimethyl-1,3,5,2,4,6-trioxatriborinane in toluene (3.5 M, 1.04 mL, 3.65 mmol) and Cs₂CO₃ (2.380 g, 7.31 mmol), and the mixture was degassed with N₂ for 5 min. After purging, Pd(dppf)Cl₂ (0.535 g, 0.73 mmol) was added, and the reaction mixture was stirred at 80° C. for 3 h. Then, the reaction mixture was diluted with H₂O (50 mL), extracted with EtOAc (3×50 mL), washed with brine (50 mL) and dried over Na₂SO₄. The solution was filtered and concentrated under reduced pressure. The crude material was purified by chromatography, eluting with a gradient of 1%-15% EtOAc in hexane, to provide (6-chloro-3-methylthieno[2,3-b]pyridin-2-yl)(3,3-difluorocyclobutyl)methyl acetate (500 mg, 1.45 mmol, 59% yield). H NMR (401 MHz, DMSO-d₆) δ 8.26 (d, J=8.5 Hz, 1H), 7.57 (d, J=8.5 Hz, 1H), 6.21 (d, J=7.0 Hz, 1H), 2.80-2.66 (m, 3H), 2.64-2.57 (m, 2H), 2.46 (s, 3H), 2.06 (s, 3H).

Step 3: (3,3-difluorocyclobutyl)(3-methyl-6-(2-methyl-2H-pyrazolo[3,4-b]pyridin-5-yl)thieno[2,3-b]pyridin-2-yl)methyl acetate. To a solution of (6-chloro-3-methylthieno[2,3-b]pyridin-2-yl)(3,3-difluorocyclobutyl)methyl acetate (500 mg, 1.45 mmol) in 1,4-dioxane (10.00 mL) and H₂O (1.00 mL) was added (2-methyl-2H-pyrazolo[3,4-b]pyridin-5-yl)boronic acid (281 mg, 1.59 mmol) and K₂CO₃ (400 mg, 2.89 mmol), and the mixture was degassed with N₂ for 5 min. After purging, SPhos Pd G3 (226 mg, 0.29 mmol) was added, and the reaction mixture was stirred at 80° C. for 1 h. Then, the reaction mixture was diluted with H₂O (25 mL), extracted with EtOAc (3×50 mL), washed with brine (50 mL), and dried over Na₂SO₄. The solution was filtered and concentrated under reduced pressure to give crude (3,3-difluorocyclobutyl)(3-methyl-6-(2-methyl-2H-pyrazolo[3,4-b]pyridin-5-yl)thieno[2,3-b]pyridin-2-yl)methyl acetate (150 mg), which was used directly for the next step without further purification. m z (ESI): 443.4 (M+H)⁺.

Step 4: (3,3-difluorocyclobutyl)(3-methyl-6-(2-methyl-2H-pyrazolo[3,4-b]pyridin-5-yl)thieno[2,3-b]pyridin-2-yl)methanol. A solution of crude (3,3-difluorocyclobutyl)(3-methyl-6-(2-methyl-2H-pyrazolo[3,4-b]pyridin-5-yl)thieno[2,3-b]pyridin-2-yl)methyl acetate (500 mg, 0.73 mmol) and K₂CO₃ (508 mg, 3.67 mmol) in MeOH (10 mL) was stirred at rt for 1 h. The reaction mixture was concentrated, diluted with H₂O (10 mL), and extracted with EtOAc (2×10 mL). The organic extracts were washed with brine (10 mL), dried over Na₂SO₄, filtered, and concentrated under reduced pressure to give a crude material. The crude material was purified by prep HPLC, eluting with a gradient of 5-95% (0.1% formic acid) H₂O in ACN with a flow rate of 2.0 mL/min, to provide Compound B83 (280 mg, 0.70 mmol, 95% yield). m/z (ESI): 401.2 (M+H)⁺. ¹H NMR (401 MHz, DMSO-d₆) δ 9.39 (d, J=2.3 Hz, 1H), 8.93 (d, J=2.3 Hz, 1H), 8.55 (s, 1H), 8.23 (d, J=8.4 Hz, 1H), 8.12 (d, J=8.5 Hz, 1H), 6.19 (d, J=4.3 Hz, 1H), 5.12 (t, J=4.6 Hz, 1H), 4.24 (s, 3H), 2.80-2.64 (m, 1H), 2.59-2.54 (m, 4H), 2.41 (s, 3H).

Step 5: SFC Purification. The sample was purified by SFC using a Chiralpak OJ-H 250×30 mm, 5 μm column, with a mobile phase of 45% (1:1) MeOH:ACN in CO₂ and using a flow rate of 130 mL/min to provide peak 1 and peak 2.

Peak 1: B83S (50 mg, 0.13 mmol, 15% yield). m/z (ESI): 401.2 (M+H)⁺. ¹H NMR (401 MHz, DMSO-d₆): δ 9.39 (d, J=2.3 Hz, 1H), 8.93 (d, J=2.3 Hz, 1H), 8.55 (s, 1H), 8.23 (d, J=8.4 Hz, 1H), 8.12 (d, J=8.5 Hz, 1H), 6.20 (d, J=4.3 Hz, 1H), 5.12 (t, J=4.6 Hz, 1H), 4.24 (s, 3H), 2.80-2.64 (m, 1H), 2.59-2.54 (m, 4H), 2.41 (s, 3H).

Peak 2: B83R (230 mg, 0.57 mmol, 68% yield). m/z (ESI): 401.2 (M+H)⁺. ¹H NMR (401 MHz, DMSO-d₆): δ 9.39 (d, J=2.3 Hz, 1H), 8.93 (d, J=2.3 Hz, 1H), 8.55 (s, 1H), 8.22 (d, J=8.5 Hz, 1H), 8.12 (d, J=8.5 Hz, 1H), 6.20 (d, J=4.3 Hz, 1H), 5.12 (t, J=4.6 Hz, 1H), 4.24 (s, 3H), 2.80-2.64 (m, 1H), 2.59-2.54 (m, 4H), 2.41 (s, 3H).

The compounds in Table 17-1 were prepared following the procedure described in Group 17, using appropriate starting materials (e.g., aldehyde/ketones and boronate esters). All starting materials are commercially available or are described in the Intermediates section above.

TABLE 17-1

| Ex. # | Chemical Structure & Name | LCMS: (ESI + ve ion) m/z; NMR | Reaction Comments |
|---|---|---|---|
| B89S | 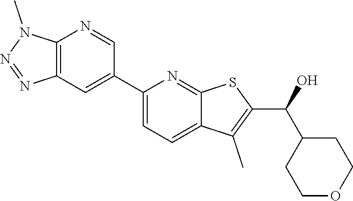 [SMILES: Cc1c([C@@H](O)C2CCOCC2)sc2nc(-c3cnc4c(c3)nnn4C)ccc12] | 396.2; ¹H NMR (400 MHz, DMSO-d₆) δ 9.58 (d, J = 1.9 Hz, 1H), 9.22 (d, J = 2.0 Hz, 1H), 8.30-8.22 (m, 2H), 5.90 (d, J = 3.4 Hz, 1H), 4.84-4.78 (m, 1H), 4.37 (s, 3H), 3.96-3.85 (m, 1H), 3.81 (dd, J = 11.3, 4.2 Hz, 1H), 3.23-3.18 (m, 2H), 2.39 (s, 3H), 1.90-1.78 (m, 2H), 1.48-1.29 (m, 2H), 1.23 (d, J = 12.7 Hz, 1H) | Step 3 was performed last; Peak 1/SFC: Chiralpak IG 250 x 21 mm, 5 μm column; Mobile phase: 50% (1:1) ACN: MeOH in CO₂ with a flow rate of 190 mL/min |
| | (S)-(3-methyl-6-(3-methyl-3H-[1,2,3]triazolo[4,5-b]pyridin- | | |
| | 6-yl)thieno[2,3-b]pyridin-2-yl)(tetrahydro-2H-pyran-4- | | |
| | yl)methanol | | |
| B89R | 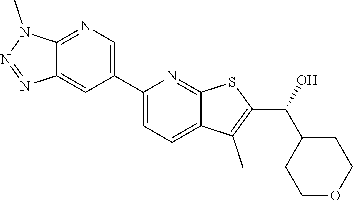 [SMILES: Cc1c([C@H](O)C2CCOCC2)sc2nc(-c3cnc4c(c3)nnn4C)ccc12] | 396.2; ¹H NMR (400 MHz, DMSO-d₆) δ 9.58 (d, J = 1.9 Hz, 1H), 9.22 (d, J = 1.9 Hz, 1H), 8.30-8.22 (m, 2H), 5.90 (s, 1H), 4.82 (d, J = 7.4 Hz, 1H), 4.37 (s, 3H), 3.90 (dd, J = 11.7, 4.1 Hz, 1H), 3.82-3.77 (m, 1H), 3.22 (dd, J = 13.3, 11.0 Hz, 2H), 2.39 (s, 3H), 1.92-1.79 (m, 2H), 1.53- 1.27 (m, 2H), 1.23 (d, J = 12.5 Hz, 1H) | Step 3 was performed last; Peak 2/SFC: Chiralpak IG 250 x 21 mm, 5 μm column; Mobile phase: 50% (1:1) ACN: MeOH in CO₂ with a flow rate of 190 mL/min |
| | (R)-(3-methyl-6-(3-methyl-3H-[1,2,3]triazolo[4,5- | | |
| | b]pyridin-6-yl)thieno[2,3-b]pyridin-2-yl)(tetrahydro-2H- | | |
| | pyran-4-yl)methanol | | |
| B90S | 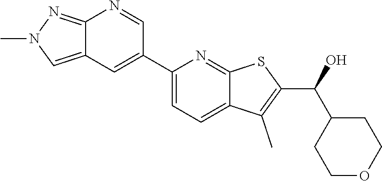 [SMILES: Cc1c([C@@H](O)C2CCOCC2)sc2nc(-c3cnc4nn(C)cc4c3)ccc12] | 395.1; ¹H NMR (401 MHz, DMSO-d₆) δ 9.39 (d, J = 2.3 Hz, 1H), 8.91 (d, J = 2.3 Hz, 1H), 8.54 (s, 1H), 8.20 (d, J = 8.4 Hz, 1H), 8.10 (d, J = 8.4 Hz, 1H), 5.86 (d, J = 2.8 Hz, 1H), 4.80 (dd, J = 7.8, 2.7 Hz, 1H), 4.24 (s, 3H), 3.90 (dd, J = 11.3, 4.3 Hz, 1H), 3.85-3.75 (m, 1H), 3.29-3.12 (m, 2H), 2.37 (s, 3H), 1.93-1.74 (m, 2H), 1.37 (dtt, J = 24.2, 11.9, | Step 3 was performed last; Peak 1/SFC: Chiralpak IG 250 x 21 mm, 5 μm column; Mobile phase: 50% (1:1) ACN:MeOH in CO₂ with a flow rate of 190 mL/min |
| | (S)-(3-methyl-6-(2-methyl-2H-pyrazolo[3,4-b]pyridin-5- | 6.2 Hz, 2H), 1.23 (d, J = 13.8 Hz, | |
| | yl)thieno[2,3-b]pyridin-2-yl)(tetrahydro-2H-pyran-4- | 1H) | |
| | yl)methanol | | |
| B90R | 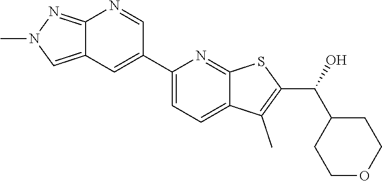 [SMILES: Cc1c([C@H](O)C2CCOCC2)sc2nc(-c3cnc4nn(C)cc4c3)ccc12] | 395.1; ¹H NMR (401 MHz, DMSO-d₆) δ 9.39 (d, J = 2.3 Hz, 1H), 8.92 (d, J = 2.3 Hz, 1H), 8.54 (s, 1H), 8.20 (d, J = 8.5 Hz, 1H), 8.10 (d, J = 8.5 Hz, 1H), 5.86 (d, J = 4.0 Hz, 1H), 4.80 (dd, J = 7.6, 4.0 Hz, 1H), 4.25 (s, 3H), 3.94-3.82 (m, 1H), 3.84-3.75 (m, 1H), 3.31-3.19 (m, 2H), 2.38 (s, 3H), 1.95-1.77 (m, 2H), 1.38 (dqd, J = 23.8, 12.0, 4.3 Hz, | Step 3 was performed last; Peak 2/SFC: Chiralpak IG 250 x 21 mm, 5 μm column; Mobile phase: 50% (1:1) ACN:MeOH in CO₂ with a flow rate of 190 mL/min |
| | (R)-(3-methyl-6-(2-methyl-2H-pyrazolo[3,4-b]pyridin-5- | 2H), 1.23 (d, J = 12.7 Hz, 1H) | |
| | yl)thieno[2,3-b]pyridin-2-yl)(tetrahydro-2H-pyran-4- | | |
| | yl)methanol | | |
| C78C | 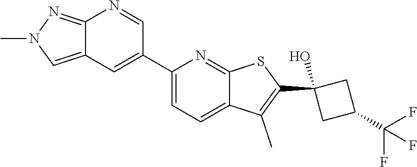 [SMILES: Cc1c2ccc(-c3cnc4nn(C)cc4c3)nc2sc1[C@]1(O)C[C@@H](C(F)(F)F)C1] | 419.2; ¹H NMR (400 MHz, DMSO-d₆) δ 9.41 (d, J = 2.3 Hz, 1H), 8.95 (d, J = 2.3 Hz, 1H), 8.55 (s, 1H), 8.27 (d, J = 8.5 Hz, 1H), 8.16 (d, J = 8.5 Hz, 1H), 6.34 (s, 1H), 4.25 (s, 3H), 3.01- 2.83 (m, 3H), 2.59 (dd, J = 11.7, 8.4 Hz, 2H), 2.46 (s, 3H) | Step 5 was not performed; |
| | cis-1-(3-methyl-6-(2-methyl-2H-pyrazolo[3,4-b]pyridin-5- | | |
| | yl)thieno[2,3-b]pyridin-2-yl)-3-(trifluoromethyl)cyclobutanol | | |
| C83C | 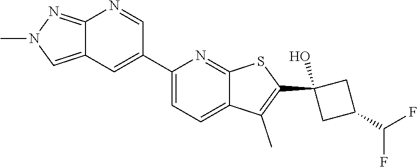 [SMILES: Cc1c2ccc(-c3cnc4nn(C)cc4c3)nc2sc1[C@]1(O)C[C@@H](C(F)F)C1] | 401.0; ¹H NMR (401 MHz, DMSO-d₆) δ 9.41 (d, J = 2.3 Hz, 1H), 8.94 (d, J = 2.3 Hz, 1H), 8.55 (s, 1H), 8.25 (d, J = 8.5 Hz, 1H), 8.15 (d, J = 8.5 Hz, 1H), 6.22 (br s, 1H) 6.19 (td, J = 56.7, 3.9 Hz, 1H), 4.25 (s, 3H), 2.80- 2.70 (m, 2H), 2.46 (s, 6H) | Step 5 was not performed; |
| | cis-3-(difluoromethyl)-1-(3-methyl-6-(2-methyl-2H- | | |
| | pyrazolo[3,4-b]pyridin-5-yl)thieno[2,3-b]pyridin-2- | | |
| | yl)cyclobutanol | | |

### Group 18

Example 18-1:1-(6-(2-methyl-2H-pyrazolo[3,4-b]pyridin-5-yl)thieno[2,3-b]pyridin-2-yl)cyclopropanol C60

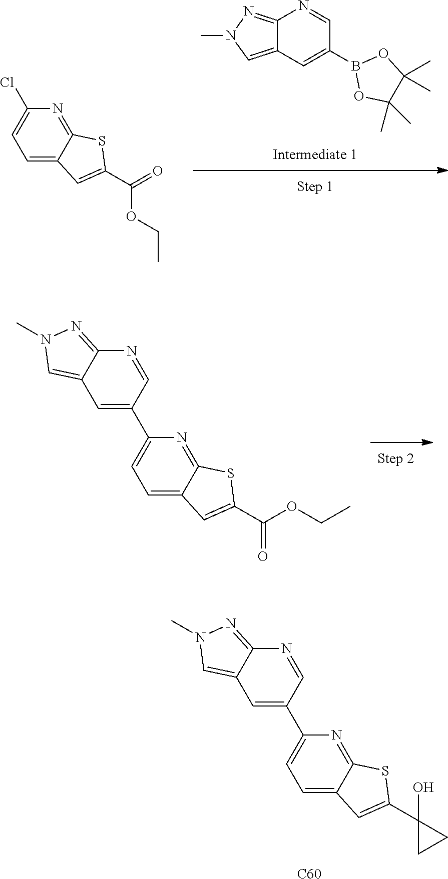

Step 1: ethyl 6-(2-methyl-2H-pyrazolo[3,4-b]pyridin-5-yl)thieno[2,3-b]pyridine-2-carboxylate. A mixture of ethyl 6-chlorothieno[2,3-b]pyridine-2-carboxylate (2 g, 8.28 mmol), Intermediate 1 (3.22 g, 12.41 mmol), and K₂CO₃ (3.43 g, 24.83 mmol) in 1,4-dioxane (40.0 mL) and H₂O (4.00 mL) was degassed with N₂ for 10 min. Then, Pd(dppf)Cl₂ (0.605 g, 0.83 mmol) was added at rt, and the reaction mixture was stirred at 105° C. for 16 h. After, the reaction mixture was cooled to rt and concentrated under reduced pressure to afford a crude material, which was washed with diethylether (2×150 mL) to afford ethyl 6-(2-methyl-2H-pyrazolo[3,4-b]pyridin-5-yl)thieno[2,3-b]pyridine-2-carboxylate (2.4 g, 7.09 mmol, 86% yield). m/z (ESI): 339.2 (M+H)⁺.

Step 2: 1-(6-(2-methyl-2H-pyrazolo[3,4-b]pyridin-5-yl)thieno[2,3-b]pyridin-2-yl)cyclopropanol. To a stirred solution of ethyl 6-(2-methyl-2H-pyrazolo[3,4-b]pyridin-5-yl)thieno[2,3-b]pyridine-2-carboxylate (0.75 g, 2.22 mmol) in THF (30.0 mL) was added titanium(IV) isopropoxide (0.909 mL, 3.10 mmol) at rt, followed by a solution of ethylmagnesium bromide in THF (2.0 M, 11.08 mL, 22.16 mmol) at −10° C. The reaction mixture was stirred at rt for 16 h. The reaction mixture was cooled to −10° C., quenched by addition of H₂O (50 mL), filtered, and extracted with 10% MeOH in DCM (2×75 mL). The combined organic layers were dried over Na₂SO₄, filtered, and concentrated under reduced pressure to give a crude material. The crude material was purified by prep HPLC, using a mobile phase of (0.1% ammonia) water in ACN with a flow rate of 15 mL/min, to afford Compound C60 (55 mg). m/z (ESI): 323.2 (M+H)⁺. ¹H NMR (401 MHz, DMSO-d₆): δ 9.37 (d, J=2.3 Hz, 1H), 8.90 (d, J=2.3 Hz, 1H), 8.54 (s, 1H), 8.18 (d, J=8.4 Hz, 1H), 8.07 (d, J=8.4 Hz, 1H), 7.09 (s, 1H), 6.69 (s, 1H), 4.24 (s, 3H), 1.33-1.26 (m, 2H), 1.19-1.11 (m, 2H).

### Group 19

Example 19-1: cis-1-(6-(2-methyl-2H-pyrazolo[3,4-b]pyridin-5-yl)thieno[2,3-b]pyridin-2-yl)-3-(trifluoromethoxy)cyclobutanol C67C

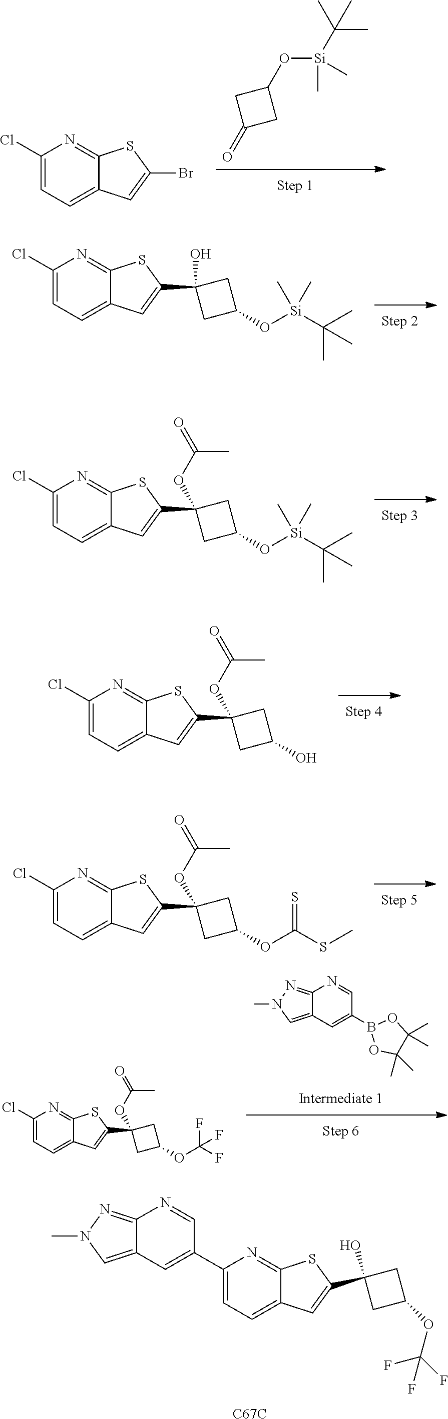

Step 1: 3-((tert-butyldimethylsilyl)oxy)-1-(6-chlorothieno[2,3-b]pyridin-2-yl)cyclobutan-1-o1. To a solution of 2-bromo-6-chlorothieno[2,3-b]pyridine (2.6 g, 10.46 mmol) and 3-((tert-butyldimethylsilyl)oxy)cyclobutan-1-one (4.19 g, 20.92 mmol) in THF (52.0 mL) was added a solution of n-butyllithium in THF (2.5 M, 10.46 mL, 26.2 mmol) in one portion at −78° C., and the resulting mixture was stirred at −78° C. for 1 h. The mixture was quenched by addition of satd. aq. NH₄Cl solution (50 mL), extracted with EtOAc (2×100 mL), washed with brine (100 mL), dried over Na₂SO₄, filtered, and concentrated under reduced pressure. The residue was purified by column chromatography, eluting with a gradient of 15-20% EtOAc in pet. ether, to give 3-((tert-butyldimethylsilyl)oxy)-1-(6-chlorothieno[2,3-b]pyridin-2-yl)cyclobutan-1-ol (2.2 g). m/z (ESI): 370.2 (M+H)⁺. ¹H NMR (401 MHz, DMSO-d₆): δ 8.21 (d, J=8.3 Hz, 1H), 7.49 (d, J=8.3 Hz, 1H), 7.38 (s, 1H), 6.41 (s, 1H), 4.19 (p, J=7.2 Hz, 1H), 2.87 (ddd, J=9.7, 7.0, 3.0 Hz, 2H), 2.39 (ddd, J=10.0, 7.5, 3.0 Hz, 2H), 0.88 (s, 9H), 0.06 (s, 6H).

Step 2: 3-((tert-butyldimethylsilyl)oxy)-1-(6-chlorothieno[2,3-b]pyridin-2-yl)cyclobutyl acetate. To a solution of 3-((tert-butyldimethylsilyl)oxy)-1-(6-chlorothieno[2,3-b]pyridin-2-yl)cyclobutan-1-ol (2.2 g, 5.95 mmol) in DCM (5 mL) at 0° C. was added acetic anhydride (3.04 g, 29.7 mmol), Et₃N (1.658 mL, 11.89 mmol) and N,N-dimethylpyridin-4-amine (0.145 g, 1.19 mmol). The reaction mass was slowly warmed to rt and stirred for 2 h. Next, the mixture was concentrated under reduced pressure, diluted with H₂O (20 mL), and extracted with DCM (20 mL×2). The combined organic layers were concentrated under reduced pressure to give crude 3-((tert-butyldimethylsilyl)oxy)-1-(6-chlorothieno[2,3-b]pyridin-2-yl)cyclobutyl acetate, which was used directly in the next step without further purification. m/z (ESI): 412.2 (M+H)⁺.

Step 3: 1-(6-chlorothieno[2,3-b]pyridin-2-yl)-3-hydroxycyclobutyl acetate. To a solution of 3-((tert-butyldimethylsilyl)oxy)-1-(6-chlorothieno[2,3-b]pyridin-2-yl)cyclobutyl acetate (2.2 g, 5.34 mmol) in THF (66.0 mL) at 0° C. was added TBAF in THF (1 M, 10.68 mL, 10.68 mmol) dropwise. The reaction mass was warmed to rt and stirred for 2 h. The reaction mixture was quenched by addition of H₂O, extracted with EtOAc, washed with H₂O, dried over Na₂SO₄, filtered, and concentrated under reduced pressure to get a crude material. The crude material was purified by column chromatography, eluting with a gradient of 50-60% EtOAc in pet. ether, to afford 1-(6-chlorothieno[2,3-b]pyridin-2-yl)-3-hydroxycyclobutyl acetate (1.2 g, 4.03 mmol, 75% yield). m/z (ESI): 298.0 (M+H)⁺. ¹H NMR (400 MHz, DMSO-d₆) δ 8.24 (d, J=8.4 Hz, 1H), 7.55-7.50 (m, 2H), 5.44 (d, J=6.4 Hz, 1H), 4.04 (p, J=7.1 Hz, 1H), 3.07 (ddd, J=10.0, 6.9, 3.2 Hz, 2H), 2.49-2.43 (m, 2H), 2.03 (s, 3H).

Step 4: 1-(6-chlorothieno[2,3-b]pyridin-2-yl)-3-(((methylthio)carbonothioyl)oxy)cyclobutyl acetate. To a solution of 1-(6-chlorothieno[2,3-b]pyridin-2-yl)-3-hydroxycyclobutyl acetate (100 mg, 0.34 mmol) in THF (2 mL) was added NaH (60% dispersion in mineral oil, 26.9 mg, 0.67 mmol) at 0° C., and the reaction mixture was stirred for 30 min. Next, a solution of CS₂ (81 μL, 1.34 mmol) in THF (5 mL) was added, and the reaction mixture was stirred at 0° C. for 20 min, followed by addition of Mel (63.0 μL, 1.01 mmol) in a dropwise manner. The reaction mixture was stirred at rt for 1 hr, quenched by addition of satd. aq. NH₄Cl (20 mL), extracted with EtOAc (2×30 mL), washed with brine (20 mL), dried over Na₂SO₄, filtered, and concentrated under reduced pressure to give a crude material. The crude material was purified by reverse phase MPLC, eluting with a mobile phase of (1:1) ACN/H₂O, to give 1-(6-chlorothieno[2,3-b]pyridin-2-yl)-3-(((methylthio)carbonothioyl)oxy)cyclobutyl acetate. m/z (ESI): 388.0 (M+H)⁺.

Step 5: 1-(6-chlorothieno[2,3-b]pyridin-2-yl)-3-(trifluoromethoxy)cyclobutyl acetate. To a solution of 1,3-dibromo-5,5-dimethylhydantoin (0.553 g, 1.93 mmol) in DCM (5.00 mL) was added HF-pyridine (1.405 g, 14.18 mmol) at −78° C., followed by addition of a solution of 1-(6-chlorothieno[2,3-b]pyridin-2-yl)-3-(((methylthio)carbonothioyl)oxy)cyclobutyl acetate (0.25 g, 0.64 mmol) in DCM (5 mL). The reaction mixture was stirred at rt for 30 min before being quenched by the addition of cold satd. aq. NaHCO₃ and extracted with DCM. The organic layer was separated, washed with H₂O, dried over Na₂SO₄, filtered, and concentrated under reduced pressure. The crude material was purified by column chromatography, eluting with a mobile phase of 20% EtOAc in pet. ether, to obtain 1-(6-chlorothieno[2,3-b]pyridin-2-yl)-3-(trifluoromethoxy)cyclobutyl acetate. m/z (ESI): 365.9 (M+H)⁺. ¹H NMR (400 MHz, DMSO-d₆):): δ (ppm) 8.30-8.21 (m, 1H), 7.68 (s, 1H), 7.56 (dd, J=8.4, 1.0 Hz, 1H), 4.83 (p, J=7.1 Hz, 1H), 3.33-3.26 (m, 2H), 2.99-2.86 (m, 2H), 2.06 (s, 3H).

Step 6: cis-1-(6-(2-methyl-2H-pyrazolo[3,4-b]pyridin-5-yl)thieno[2,3-b]pyridin-2-yl)-3-(trifluoromethoxy)cyclobutan-1-ol. A stirred solution of 1-(6-chlorothieno[2,3-b]pyridin-2-yl)-3-(trifluoromethoxy)cyclobutyl acetate (40 mg, 0.11 mmol), Intermediate 1 (36.8 mg, 0.14 mmol) and K₂CO₃ (45.3 mg, 0.33 mmol) in 1,4-dioxane (1.44 mL) and H₂O (0.16 mL) was purged with N₂ gas for 5 min. After purging, SPhos Pd G3 (8.53 mg, 10.94 μmol) was added. The reaction mixture was stirred at 110° C. for 3 h. After, the reaction mass was diluted with EtOAc, filtered through celite and washed with EtOAc (50 mL). The filtrate was concentrated under reduced pressure. The crude material was purified by reverse-phase preparative HPLC, eluting with a linear gradient of 0.1%(NH₃) H₂O in ACN and a flow rate of 15 mL/min, to afford Compound C67C. m/z (ESI): 421.0 (M+H)⁺. ¹H NMR (400 MHz, DMSO-d₆) δ (ppm) 9.38 (d, J=2.4 Hz, 1H), 8.92 (d, J=2.4 Hz, 1H), 8.55 (s, 1H), 8.25 (d, J=8.4 Hz, 1H), 8.11 (d, J=8.3 Hz, 1H), 7.49 (s, 1H), 6.73 (s, 1H), 4.79 (p, J=7.2 Hz, 1H), 4.25 (s, 3H), 3.08 (ddd, J=10.5, 7.2, 3.2 Hz, 2H), 2.73 (ddd, J=10.3, 7.2, 3.0 Hz, 2H).

Example 19-2: cis-1-(2-(2-methyl-2H-pyrazolo[3,4-b]pyridin-5-yl)thieno[2,3-d]pyrimidin-6-yl)-3-(trifluoromethoxy)cyclobutanol C100C

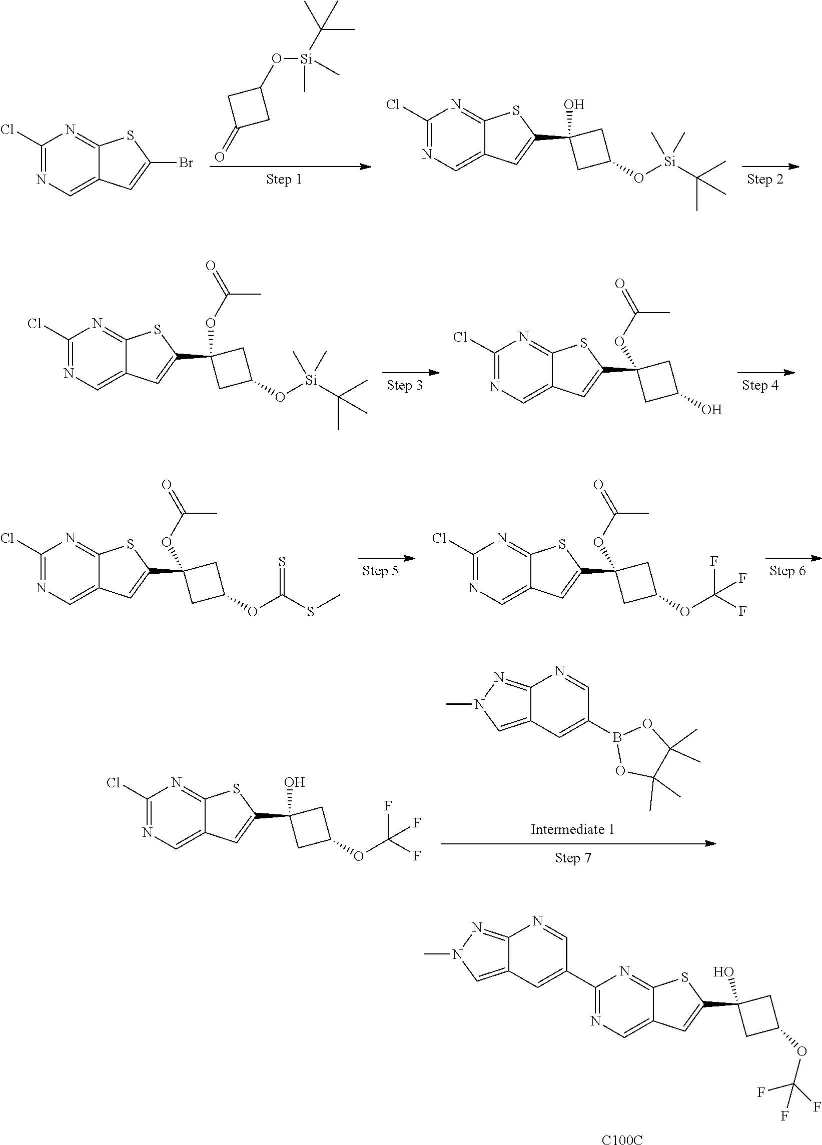

Step 1: cis-3-((tert-butyldimethylsilyl)oxy)-1-(2-chlorothieno[2,3-dipyrimidin-6-yl)cyclobutan-1-ol. To a solution of 6-bromo-2-chlorothieno[2,3-d]pyrimidine (0.9 g, 3.61 mmol) in toluene (90 mL) at −78° C. was added n-butyllithium (2.5 M in hexane, 2.16 mL, 5.41 mmol). The mixture was stirred for 2 minutes at which time a solution of 3-((tert-butyldimethylsilyl)oxy)cyclobutan-1-one (1.084 g, 5.41 mmol) in THF (10 mL) was added. The reaction mixture was stirred for 15 minutes at −78° C. Then, satd. aq. NH₄Cl solution (75 mL) was added. The aqueous layer was extracted with EtOAc (2×75 mL), dried over Na₂SO₄, filtered, and concentrated in vacuo. The residue was purified by column chromatography, eluting with a gradient of 5-15% EtOAc in hexane, to provide cis-3-((tert-butyldimethylsilyl)oxy)-1-(2-chlorothieno[2,3-d]pyrimidin-6-yl)cyclobutan-1-ol (0.5 g, 1.348 mmol, 37.4% yield). m/z (ESI): 371.2 (M+H)⁺. ¹H NMR (400 MHz, DMSO-d₆): δ (ppm) 9.12 (s, 1H), 7.51 (s, 1H), 6.58 (d, J=1.7 Hz, 1H), 4.22 (p, J=7.3 Hz, 1H), 2.88 (ddd, J=9.8, 7.0, 2.9 Hz, 2H), 2.41 (ddd, J=12.2, 6.3, 2.6 Hz, 2H), 0.88 (s, 9H), 0.06 (d, J=1.5 Hz, 6H).

Step 2: cis-3-((tert-butyldimethylsilyl)oxy)-1-(2-chlorothieno[2,3-d]pyrimidin-6-yl)cyclobutyl acetate. To a solution of cis-3-((tert-butyldimethylsilyl)oxy)-1-(2-chlorothieno[2,3-d]pyrimidin-6-yl)cyclobutan-1-ol (2.20 g, 5.95 mmol) in DCM (30 mL) at 0° C. was added acetic anhydride (1.27 mL, 13.5 mmol), followed by TEA (0.751 mL, 5.39 mmol) and DMAP (0.066 g, 0.539 mmol). The reaction mass was slowly allowed to reach rt and stirred for 2 h. Then, the mixture was concentrated in vacuo to give a residue. The residue was diluted with water (50 mL), extracted with EtOAc (2×50 mL), washed with brine (50 mL), dried over Na₂SO₄, filtered, and concentrated in vacuo to provide crude material. The crude material was purified by chromatography, eluting with a gradient of 10-30% EtOAc in hexane, to provide cis-3-((tert-butyldimethylsilyl)oxy)-1-(2-chlorothieno[2,3-d]pyrimidin-6-yl)cyclobutyl acetate (0.95 g, 2.30 mmol, 85% yield). m/z (ESI): 413.1 (M+H)⁺. ¹H NMR (400 MHz, DMSO-d₆) δ (ppm) 9.18 (s, 1H), 7.72 (s, 1H), 4.23 (p, J=7.2 Hz, 1H), 3.17 (ddd, J=10.0, 6.8, 3.1 Hz, 2H), 2.58-2.52 (m, 2H), 2.04 (s, 3H), 0.87 (s, 9H), 0.06 (s, 6H).

Step 3: cis-1-(2-chlorothieno[2,3-d]pyrimidin-6-yl)-3-hydroxycyclobutyl acetate. To a mixture of cis-3-((tert-butyldimethylsilyl)oxy)-1-(2-chlorothieno[2,3-d]pyrimidin-6-yl)cyclobutyl acetate (1.93 g, 8.92 mmol) in THF (38.0 mL) was slowly added TBAF (1.0 M in THF, 4.60 mL, 4.60 mmol) at 0° C. under an atmosphere of N₂. The mixture was allowed to warm to rt and was stirred at 25° C. for 2 h. Then, the reaction mixture was diluted with satd. aq. NH₄Cl (50 mL), extracted with EtOAc (2×30 mL), washed with brine (50 mL), dried over Na₂SO₄, filtered, and concentrated in vacuo to give crude material. The crude material was purified by chromatography, eluting with a gradient of 30-50% EtOAc in hexane, to provide cis-1-(2-chlorothieno[2,3-d]pyrimidin-6-yl)-3-hydroxycyclobutyl acetate (0.48 g, 1.61 mmol, 70% yield). m/z (ESI): 299.0 (M+H)⁺. ¹H NMR (400 MHz, DMSO-d₆) δ (ppm) 9.16 (s, 1H), 7.66 (s, 1H), 5.48 (d, J=6.4 Hz, 1H), 4.12-4.01 (m, 1H), 3.18-3.07 (m, 2H), 2.44-2.49 (m, 2H), 2.04 (s, 3H).

Step 4: cis-1-(2-chlorothieno[2,3-d]pyrimidin-6-yl)-3-(((methylthio)carbonothioyl)oxy)cyclobutyl acetate. To a solution of cis-1-(2-chlorothieno[2,3-d]pyrimidin-6-yl)-3-hydroxycyclobutyl acetate (0.43 g, 1.44 mmol) in THF (22 mL) was added NaH (60 wt. % dispersion in mineral oil, 0.115 g, 2.88 mmol) at 0° C. The reaction mixture was stirred for 30 minutes at 0° C., at which time CS₂ (0.347 mL, 5.76 mmol) in THF (3 ml) was added. The reaction mixture was stirred at 0° C. for 20 min, followed by addition of Mel (0.270 mL, 4.32 mmol) in THF (3 ml) dropwise. The reaction mixture was stirred at 0° C. for 1 h. The reaction mixture was diluted with ice cold water (50 mL), extracted with EtOAc (2×50 mL), washed with brine (50 mL), dried over Na₂SO₄, filtered, and concentrated in vacuo to give a crude material. The crude material was purified by chromatography, eluting with a gradient of 10-20% EtOAc in hexane, to provide cis-1-(2-chlorothieno[2,3-d]pyrimidin-6-yl)-3-(((methylthio)carbonothioyl)oxy)cyclobutyl acetate (0.38 g, 0.977 mmol, 68% yield). m/z (ESI): 389.0 (M+H)⁺. ¹H NMR (400 MHz, DMSO-d₆) δ (ppm) 9.21 (s, 1H), 7.77 (s, 1H), 5.46 (p, J=7.2 Hz, 1H), 3.39 (ddd, J=10.6, 7.0, 3.2 Hz, 2H), 2.95 (ddd, J=10.6, 7.2, 3.3 Hz, 2H), 2.60 (s, 3H), 2.07 (s, 3H).

Step 5: cis-1-(2-chlorothieno[2,3-d]pyrimidin-6-yl)-3-(trifluoromethoxy)cyclobutyl acetate. To a solution of 1,3-dibromo-5,5-dimethylhydantoin (0.838 g, 2.93 mmol) in dichloromethane (19 mL) at −78° C. was added HF-pyridine (2.13 g, 21.5 mmol). This reaction was stirred for 5 minutes at which time cis-1-(2-chlorothieno[2,3-d]pyrimidin-6-yl)-3-(((methylthio)carbonothioyl)oxy)cyclobutyl acetate (0.38 g, 0.977 mmol) in DCM (10 ml) was added. The reaction mixture was stirred at rt for 30 minutes. Then, the reaction mixture was slowly quenched with ice cold satd. aq. NaHCO₃, extracted with DCM (2×50 mL), washed with brine (50 mL), dried over Na₂SO₄, filtered, and concentrated under reduced pressure to give a crude material. The crude material was purified by chromatography, eluting with a gradient of 5-15% EtOAc in DCM, to provide cis-1-(2-chlorothieno[2,3-d]pyrimidin-6-yl)-3-(trifluoromethoxy)cyclobutyl acetate (0.28 g, 0.763 mmol, 78% yield). m/z (ESI): 367.0 (M+H)⁺. ¹H NMR (400 MHz, DMSO-d₆) δ (ppm) 9.19 (s, 1H), 7.82 (s, 1H), 4.83 (p, J=7.2 Hz, 1H), 3.40-3.32 (m, 2H), 2.90-3.00 (m, 2H), 2.07 (s, 3H).

Step 6: cis-1-(2-chlorothieno[2,3-d]pyrimidin-6-yl)-3-(trifluoromethoxy)cyclobutan-1-ol. To a solution of cis-1-(2-chlorothieno[2,3-d]pyrimidin-6-yl)-3-(trifluoromethoxy)cyclobutyl acetate (0.3 g, 0.818 mmol) at rt in methanol (9 mL) and water (0.9 mL) was added potassium carbonate (0.339 g, 2.45 mmol). The reaction mass was stirred at rt for 1 h. Then, the solvent was concentrated in vacuo to provide a residue. The residue was diluted with water (25 mL), extracted with EtOAc (30 mL×3), washed with brine (30 mL), dried over Na₂SO₄, filtered, and concentrated under reduced pressure. The crude material purified by chromatography, eluting with a gradient of 15-30% EtOAc in hexane, to provide cis-1-(2-chlorothieno[2,3-d]pyrimidin-6-yl)-3-(trifluoromethoxy)cyclobutan-1-ol (0.22 g, 0.678 mmol, 83% yield). m/z (ESI): 325.0. ¹H NMR (400 MHz, DMSO-d₆) δ (ppm) 9.13 (s, 1H), 7.65 (s, 1H), 6.92 (s, 1H), 4.82 (p, J=7.2 Hz, 1H), 3.06 (ddd, J=10.5, 7.2, 3.2 Hz, 2H), 2.73 (ddd, J=10.5, 7.3, 3.2 Hz, 2H).

Step 7: cis-1-(2-(2-methyl-2H-pyrazolo[3,4-b]pyridin-5-yl)thieno[2,3-d]pyrimidin-6-yl)-3-(trifluoromethoxy)cyclobutanol. A mixture of cis-1-(2-chlorothieno[2,3-d]pyrimidin-6-yl)-3-(trifluoromethoxy)cyclobutan-1-ol (0.3 g, 0.818 mmol), Intermediate 1 (0. g, 0.462 mmol), and K₂CO₃ (0.128 g, 0.924 mmol) in 1,4-dioxane (3.6 mL) and water (0.4 mL) was purged with N₂ gas for 5 min. Then, SPhos Pd G3 (0.024 g, 0.031 mmol) was added. The reaction mixture was stirred at 110° C. for 2 h. After, the reaction mass was diluted with EtOAc, filtered through celite and washed with EtOAc (50 mL). The filtrate was washed with satd. aq. NH₄Cl (50 mL), dried over Na₂SO₄, filtered, and concentrated in vacuo to provide crude material. The crude material was purified by reverse-phase preparative HPLC, eluting with a linear gradient of 0.1% ammonia in water in ACN, to provide Compound C100C (0.185 mmol, 60% yield). m/z (ESI): 422.2 (M+H)⁺. ¹H NMR (400 MHz, DMSO-d₆): δ (ppm) 9.64 (d, J=2.2 Hz, 1H), 9.29 (s, 1H), 9.23 (d, J=2.2 Hz, 1H), 8.59 (s, 1H), 7.63 (s, 1H), 6.87 (s, 1H), 4.82 (q, J=7.2 Hz, 1H), 4.25 (s, 3H), 3.10 (ddd, J=10.4, 7.2, 3.1 Hz, 2H), 2.75 (ddd, J=10.4, 7.2, 3.2 Hz, 2H). ¹⁹F NMR (377 MHz, DMSO-d₆) δ -57.81.

Alternate conditions in reference to Example 19-1:
(1) Prior to Step 6: To a solution of the product from step 5 (0.82 mmol) in MeOH (9.0 mL) and H₂O (0.90 mL), was added K₂CO₃ at rt, and the reaction was stirred for 1 h at rt. Then, the solution was concentrated, diluted with H₂O (25 mL), and extracted with EtOAc (30 mL×3). The combined organic layers were washed with brine (30 mL), dried over Na₂SO₄, and concentrated. The crude material was purified by chromatography, eluting with a gradient of 15% o to 30% o EtOAc in hexane, to provide the product.
(2) Step 1 was performed in toluene instead of THF.
(3) In substitution of Step 1: To a solution of 5-chlorothieno[3,2-b]pyridine (0.590 mmol) in tetrahydrofuran (5 mL) at −78C was added n-butyllithium (2.5 M in Hexane, 1.179 mmol) dropwise over 5 minutes, and the mixture was stirred for 15 min 3-((tert-butyldimethylsilyl)oxy)cyclobutan-1-one (0.154 g, 0.766 mmol) was added dropwise at −78° C. over 5 minutes, and the recation mixture was stirred for an additional 15 min. The reaction mixture was slowly quenched with sat. aq. NH₄Cl and extracted with EtOAc (2×20 mL). The combined organic layers were washed with brine, dried over Na₂SO₄, filtered and concentrated under reduced pressure. The crude residue was purified by column chromatography, eluting with 0-20% o EtOAc in pet. ether, to afford cis-3-((tert-butyldimethylsilyl)oxy)-1-(5-chlorothieno[3,2-b]pyridin-2-yl)cyclobutan-1-ol.

The compounds in Table 19-1 were prepared following the procedure described in Group 19, using appropriate starting materials (e.g., aldehyde/ketones and boronic esters). All starting materials are commercially available or are described in the Intermediates section above.

TABLE 19-1

| | | | Reaction |
|---|---|---|---|
| Ex. # | Chemical Structure & Name | LCMS: (ESI + ve ion) m/z; NMR | Comments |
| C101C | 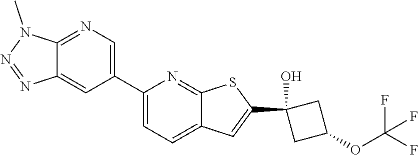 [SMILES: Cn1nnc2cc(-c3ccc4cc([C@]5(O)C[C@@H](OC(F)(F)F)C5)sc4n3)cnc21] | 422.2; ¹H NMR (401 MHz, DMSO- d₆) δ 9.58 (d, J = 2.0 Hz, 1H), 9.22 (d, J = 2.0 Hz, 1H), 8.31 (d, J = 8.4 Hz, 1H), 8.25 (d, J = 8.4 Hz, 1H), 7.53 (s, 1H), 6.74 (s, 1H), 4.81 (p, J = 7.2 Hz, 1H), 4.37 (s, 3H), 3.09 (ddd, J = 10.4, 7.3, 3.1 Hz, 2H), 2.74 (ddd, J = 10.4, 7.4, 3.1 Hz, 2H) ¹⁹F NMR (377 MHz, DMSO-d₆) δ- | Alternate condition (2) was used |
| | cis-1-(6-(3-methyl-3H-[1,2,3]triazolo[4,5-b]pyridin-6- | 57.78 | |
| | yl)thieno[2,3-b]pyridin-2-yl)-3-(trifluoromethoxy)cyclobutanol | | |
| C105C | 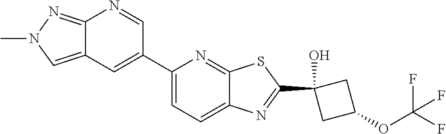 [SMILES: Cn1cc2cc(-c3ccc4nc([C@]5(O)C[C@@H](OC(F)(F)F)C5)sc4n3)cnc2n1] | 422.2; ¹H NMR (401 MHz, DMSO- d₆) δ 9.39 (d, J = 2.3 Hz, 1H), 8.96 (d, J = 2.3 Hz, 1H), 8.57 (s, 1H), 8.51 (d, J = 8.6 Hz, 1H), 8.27 (d, J = 8.7 Hz, 1H), 7.34 (s, 1H), 4.98 (p, J = 7.2 Hz, 1H), 4.25 (s, 3H), 3.20- 3.10 (m, 2H), 2.82-2.73 (m, 2H) ¹⁹F NMR (377 MHz, DMSO-d₆) δ- | Alternate condition (1) was used |
| | cis-1-(5-(2-methyl-2H-pyrazolo[3,4-b]pyridin-5-yl)[1,3]thiazolo[5,4-b] | 57.93 | |
| | pyridin-2-yl)-3-(trifluoromethoxy)cyclobutanol | | |
| C111C | 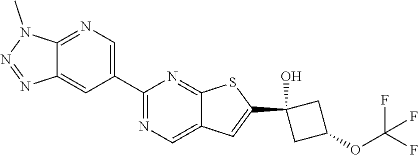 [SMILES: Cn1nnc2cc(-c3ncc4cc([C@]5(O)C[C@@H](OC(F)(F)F)C5)sc4n3)cnc21] | 423.0; ¹H NMR (400 MHz, DMSO- d₆) δ 9.82 (d, J = 1.9 Hz, 1H), 9.42- 9.30 (m, 2H), 7.69 (s, 1H), 6.90 (s, 1H), 4.84 (q, J = 7.2 Hz, 1H), 4.38 (s, 3H), 3.16-3.06 (m, 2H), 2.82-2.72 (m, 2H) | Alternate conditions (1) and (2) were used |
| | cis-1-(2-(3-methyl-3H-[1,2,3]triazolo[4,5-b]pyridin-6- | | |
| | yl)thieno[2,3-d]pyrimidin-6-yl)-3-(trifluoromethoxy)cyclobutanol | | |
| C112C | 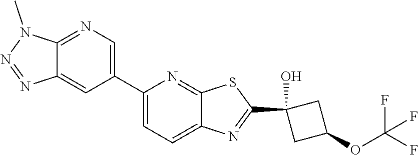 [SMILES: Cn1nnc2cc(-c3ccc4nc([C@]5(O)C[C@H](OC(F)(F)F)C5)sc4n3)cnc21] | 423.2; ¹H NMR (400 MHz, DMSO- d₆) δ 9.59 (d, J = 2.0 Hz, 1H), 9.26 (d, J = 2.0 Hz, 1H), 8.58 (d, J = 8.6 Hz, 1H), 8.41 (d, J = 8.7 Hz, 1H), 7.37 (s, 1H), 4.99 (p, J = 7.1 Hz, 1H), 4.37 (s, 3H), 3.16 (ddd, J = 10.2, 7.2, 3.0 Hz, 2H), 2.84-2.74 (m, 2H) | Alternate condition (1) was used |
| | cis-1-(5-(3-methyl-3H-[1,2,3]triazolo[4,5-b]pyridin-6-yl)[1,3] | | |
| | thiazolo[5,4-b]pyridin-2-yl)-3-(trifluoromethoxy)cyclobutanol | | |
| C117C | 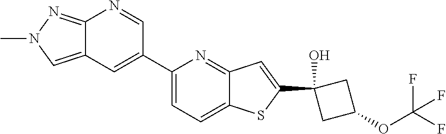 [SMILES: Cn1cc2cc(-c3ccc4sc([C@]5(O)C[C@@H](OC(F)(F)F)C5)cc4n3)cnc2n1] | 421.2; ¹H NMR (400 MHz, DMSO- d₆) δ 9.42 (d, J = 2.3 Hz, 1H), 8.89 (d, J = 2.3 Hz, 1H), 8.56 (s, 1H), 8.50 (d, J = 8.5 Hz, 1H), 8.01 (d, J = 8.6 Hz, 1H), 7.71 (s, 1H), 6.75 (s, 1H), 4.91 (q, J = 7.2 Hz, 1H), 4.25 (s, 3H), 3.11 (ddd, J = 10.4, 7.2, 3.1 Hz, 2H), 2.78-2.71 (m, 2H) | Alternate condition (1) and (3) were used |
| | cis-1-(5-(2-methyl-2H-pyrazolo[3,4-b]pyridin-5- | | |
| | yl)thieno[3,2-b]pyridin-2-yl)-3-(trifluoromethoxy)cyclobutanol | | |

### Group 20

Example 20-1: trans-3-fluoro-1-(6-(2-methyl-2H-pyrazolo[3,4-b]pyridin-5-yl)thieno[2,3-b]pyridin-2-yl)cyclobutanol C40T

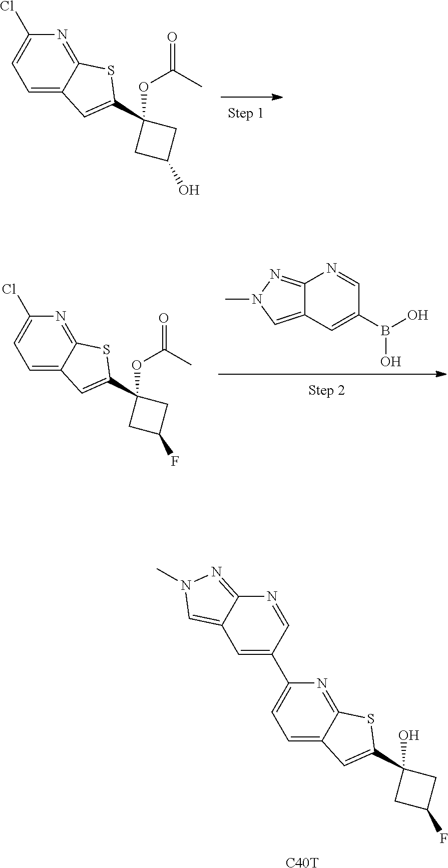 [SMILES: CC(=O)O[C@]1(c2cc3ccc(Cl)nc3s2)C[C@H](O)C1 ; CC(=O)O[C@]1(c2cc3ccc(Cl)nc3s2)C[C@@H](F)C1 ; Cn1cc2cc(B(O)O)cnc2n1 ; "C40T" ; Cn1cc2cc(-c3ccc4cc([C@]5(O)C[C@H](F)C5)sc4n3)cnc2n1]

Step 1: 1-(6-chlorothieno[2,3-b]pyridin-2-yl)-3-fluorocyclobutyl acetate. To a solution of 1-(6-chlorothieno[2,3-b]pyridin-2-yl)-3-hydroxycyclobutyl acetate (0.9 g, 3.02 minol) in DCM (2 mL) at -10° C. was added a solution of DAST (0.799 mL, 6.05 minol) in DCM (1 mL). The reaction mass was stirred for 1 h at 0° C. before being warmed to rt and stirred for 12 h. After, the reaction was quenched by addition of 10% aqueous NaHCO₃ (2 mL), extracted with DCM (3×150 mL), washed with brine (50 mL), dried over Na₂SO₄, filtered, and concentrated under reduced pressure. The crude material was purified by column chromatography, eluting with a mobile phase of 15-20% EtOAc in pet. ether, to give 1-(6-chlorothieno[2,3-b]pyridin-2-yl)-3-fluorocyclobutyl acetate (0.4 g, 1.33 mmol, 44% yield). m z (ESI): 300.0 (M+H)⁺. ¹H NMR (401 MHz, DMSO-d₆): δ 8.27 (d, J=8.4 Hz, 1H), 7.59-7.51 (m, 2H), 5.44 (tt, J=6.7, 4.5 Hz, 1H), 3.21-3.06 (m, 2H), 3.00-2.85 (m, 2H), 2.06 (s, 3H).

Step 2: trans-3-fluoro-1-(6-(2-methyl-2H-pyrazolo[3,4-b]pyridin-5-yl)thieno[2,3-b]pyridin-2-yl)cyclobutanol. A mixture of 1-(6-chlorothieno[2,3-b]pyridin-2-yl)-3-fluorocyclobutyl acetate (0.05 g, 0.17 mmol), (2-methyl-2H-pyrazolo[3,4-b]pyridin-5-yl)boronic acid (0.038 g, 0.22 mmol), and K₂CO₃ (0.069 g, 0.50 mmol) in 1,4-dioxane (1.0 mL) and H₂O (0.10 mL) was degassed with N₂ for 10 min. SPhos Pd G3 (0.015 g, 0.017 mmol) was added at rt, and the reaction mixture was stirred at 80° C. for 1 h. Then, the reaction was cooled to rt, filtered and concentrated under reduced pressure. The crude material was purified by prep HPLC, eluting with a gradient of 5-95% (0.1% formic acid) H₂O in ACN with a flow rate of 2.0 mL/min, to afford Compound C40T. m/z (ESI): 355.0 (M+H)⁺. ¹H NMR (401 MHz, DMSO-d₆): δ 9.39 (d, J=2.3 Hz, 1H), 8.92 (d, J=2.4 Hz, 1H), 8.54 (s, 1H), 8.28 (d, J=8.4 Hz, 1H), 8.10 (d, J=8.4 Hz, 1H), 7.42 (s, 1H), 6.47 (s, 1H), 5.47 (p, J=6.3 Hz, 1H), 4.24 (s, 3H), 2.89-2.64 (m, 4H).

### Group 21

Example 21-1: (R)-(3,3-difluorocyclobutyl)(5-(2-methyl-2H-pyrazolo[3,4-b]pyridin-5-yl)thieno[2,3-b]pyridin-2-yl)methanol B77R; (S)-(3,3-difluorocyclobutyl)(5-(2-methyl-2H-pyrazolo[3,4-b]pyridin-5-yl)thieno[2,3-b]pyridin-2-yl)methanol B77S

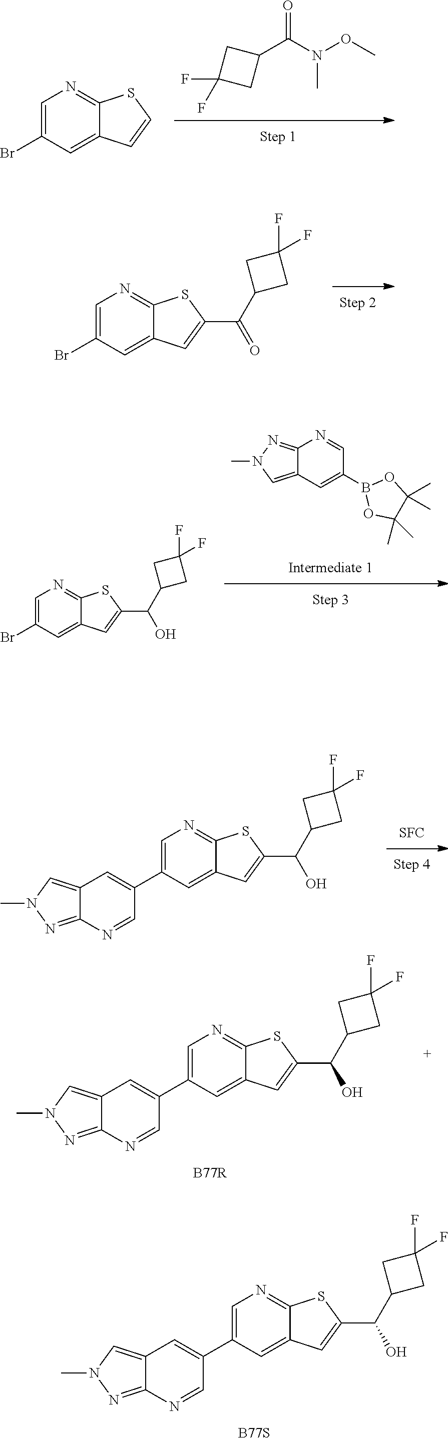

Step 1: (5-bromothieno[2,3-b]pyridin-2-yl)(3,3-difluorocyclobutyl)methanone. To a solution of 5-bromothieno[2,3-b]pyridine (1 g, 4.67 mmol) in THF (20 mL) at −78° C. was added a solution of LDA in THF (2.0 M, 4.67 mL, 9.34 mmol) dropwise over 5 min, and the reaction mixture was stirred for 1 h at the same temperature. Next, 3,3-difluoro-N-methoxy-N-methylcyclobutane-1-carboxamide (1.674 g, 9.34 mmol) in THF (2 mL) was added dropwise over 5 min at −78° C., and the reaction mixture was stirred for 1 h at the same temperature. Then, the reaction mixture was slowly quenched by addition of sat. aq. NH₄C₁, extracted with EtOAc, washed with H₂O, dried over Na₂SO₄, filtered, and concentrated under reduced pressure. The crude material was purified by column chromatography, eluting with a gradient of 20-30% EtOAc in pet. ether, to afford (5-bromothieno[2,3-b]pyridin-2-yl)(3,3-difluorocyclobutyl)methanone (0.8 g, 2.41 mmol, 52% yield). m/z (ESI): 332.0 (M+H)⁺. ¹H NMR (401 MHz, DMSO-d₆) δ 8.85 (d, J=2.3 Hz, 1H), 8.71 (d, J=2.3 Hz, 1H), 8.32 (s, 1H), 4.12 (pd, J=8.5, 2.9 Hz, 1H), 3.07-2.86 (m, 4H).

Step 2: (5-bromothieno[2,3-b]pyridin-2-yl)(3,3-difluorocyclobutyl)methanol. To a solution of (5-bromothieno[2,3-b]pyridin-2-yl)(3,3-difluorocyclobutyl)methanone (0.8 g, 2.41 mmol) in MeOH (16 mL) at 0° C. was added NaBH₄ (0.182 g, 4.82 mmol), and the reaction was stirred at 0° C. for 15 min. After, the reaction mixture was quenched by addition of H₂O (50 mL) and concentrated under reduced pressure. The residue was diluted with H₂O (100 mL), extracted with EtOAc (2×50 mL), washed with brine (100 mL), dried over Na₂SO₄, filtered, and concentrated under reduced pressure. The crude material was purified by chromatography, eluting with a gradient of 1-60% EtOAc in hexane, to provide (5-bromothieno[2,3-b]pyridin-2-yl)(3,3-difluorocyclobutyl)methanol (0.7 g, 2.10 mmol, 87% yield). m z (ESI): 334.0 (M+H)⁺. ¹H NMR (401 MHz, DMSO-d₆) δ 8.59 (d, J=2.2 Hz, 1H), 8.46 (d, J=2.3 Hz, 1H), 7.28 (d, J=1.0 Hz, 1H), 6.31 (d, J=5.1 Hz, 1H), 4.93 (t, J=5.0 Hz, 1H), 2.60-2.54 (m, 5H).

Step 3: (3,3-difluorocyclobutyl)(5-(2-methyl-2H-pyrazolo[3,4-b]pyridin-5-yl)thieno[2,3-b]pyridin-2-yl)methanol. To a stirred solution of (5-bromothieno[2,3-b]pyridin-2-yl)(3,3-difluorocyclobutyl)methanol (0.7 g, 2.10 mmol) in 1,4-dioxane (14 mL) and H₂O (1.40 mL) was added Intermediate 1 (0.651 g, 2.51 mmol) followed by K₂CO₃ (0.868 g, 6.28 mmol), and the mixture was purged with N₂ gas for 5 min. After purging, Pd(dppf)Cl₂ (0.153 g, 0.21 mmol) was added, and the reaction mixture was stirred at 100° C. for 2 h. Next, the reaction mixture was diluted with H₂O (100 mL), extracted with EtOAc (2×100 mL), washed with brine (100 mL), dried over Na₂SO₄, filtered, and concentrated under reduced pressure. The crude material was recrystallized with 20% EtOAc in pet. ether and filtered to provide (3,3-difluorocyclobutyl)(5-(2-methyl-2H-pyrazolo[3,4-b]pyridin-5-yl)thieno[2,3-b]pyridin-2-yl)methanol (0.7 g, 1.81 mmol, 86% yield). m/z (ESI): 387.1 (M+H)⁺. ¹H NMR (401 MHz, DMSO-d₆) δ 8.99 (d, J=2.4 Hz, 1H), 8.88 (d, J=2.2 Hz, 1H), 8.58 (d, J=2.4 Hz, 1H), 8.53 (br s, 2H), 7.37 (s, 1H), 6.28 (dd, J=5.1, 1.4 Hz, 1H), 4.96 (t, J=4.8 Hz, 1H), 4.25 (s, 3H), 2.62-2.53 (m, 5H).

Step 4: SFC Purification. The sample was purified by SFC using a Chiralpak IB 250×30 mm, 5 μm column, with a mobile phase of 40% MeOH in CO₂ and using a flow rate of 170 mL/min to provide peak 1 and peak 2.

Peak 1: B77R (0.315 g, 0.82 mmol, 45% yield). m/z (ESI): 387.2 (M+H)⁺. ¹H NMR (401 MHz, DMSO-d₆): δ 8.99 (d, J=2.3 Hz, 1H), 8.88 (d, J=2.2 Hz, 1H), 8.57 (d, J=2.4 Hz, 1H), 8.53 (b s, 2H), 7.37 (s, 1H), 6.28 (d, J=4.1 Hz, 1H), 4.96 (t, J=4.1 Hz, 1H), 4.25 (s, 3H), 2.62-2.53 (m, 5H).

Peak 2: B77S (0.310 g, 0.80 mmol, 44% yield). m/z (ESI): 387.2 (M+H)⁺. ¹H NMR (401 MHz, DMSO-d₆): δ 8.99 (d, J=2.5 Hz, 1H), 8.88 (d, J=2.2 Hz, 1H), 8.58 (d, J=2.4 Hz, 1H), 8.53 (b s, 2H), 7.37 (s, 1H), 6.28 (d, J=5.1 Hz, 1H), 4.96 (t, J=4.8 Hz, 1H), 4.25 (s, 3H), 2.62-2.53 (m, 5H)..

### Group 22

Example 22-1:3,3-difluoro-1-(3-methyl-6-(2-methyl-2H-pyrazolo[3,4-b]pyridin-5-yl)thieno[2,3-b]pyridin-2-yl)cyclobutanol C77

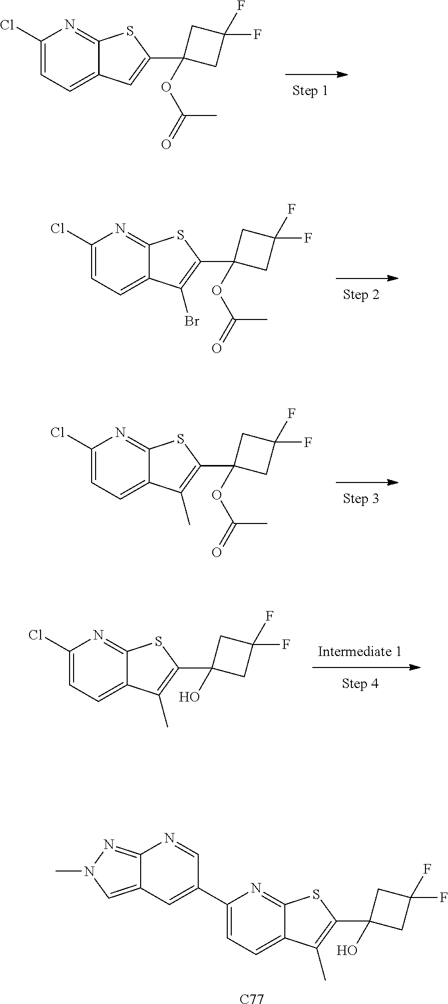 [SMILES: CC(=O)OC1(c2cc3ccc(Cl)nc3s2)CC(F)(F)C1 ; CC(=O)OC1(c2sc3nc(Cl)ccc3c2Br)CC(F)(F)C1 ; CC(=O)OC1(c2sc3nc(Cl)ccc3c2C)CC(F)(F)C1 ; Cc1c(C2(O)CC(F)(F)C2)sc2nc(Cl)ccc12 ; Cc1c(C2(O)CC(F)(F)C2)sc2nc(-c3cnc4nn(C)cc4c3)ccc12 ; "C77"]

Step 1: 1-(3-bromo-6-chlorothieno[2,3-b]pyridin-2-yl)-3,3-difluorocyclobutyl acetate. To a sealed tube was added 1-(6-chlorothieno[2,3-b]pyridin-2-yl)-3,3-difluorocyclobutyl acetate (0.8 g, 2.52 mmol) and N-bromosuccinimide (15.68 g, 88 mmol) in DMF (50 mL). The reaction mixture was stirred at 70° C. for 16 h, then the reaction mixture was cooled to rt. The reaction mixture was diluted with H₂O (100 mL) and extracted with EtOAc (2×50 mL). The combined organic extracts were washed with satd. aq. NaCl (2×50 mL), dried over Na₂SO₄, filtered, and concentrated. The crude material was purified by chromatography, eluting with a gradient of 2%-10% EtOAc in hexane, to provide 1-(3-bromo-6-chlorothieno[2,3-b]pyridin-2-yl)-3,3-difluorocyclobutyl acetate (0.55 g, 1.4 mmol, 55% yield). m/z (ESI): 396.0 (M+H)⁺. ¹H NMR (400 MHz, DMSO-d₆) δ 8.20 (d, J=8.5 Hz, 1H), 7.68 (d, J=8.5 Hz, 1H), 3.64-3.48 (m, 4H), 2.05 (s, 3H).

Step 2: 1-(6-Chloro-3-methylthieno[2,3-b]pyridin-2-yl)-3,3-difluorocyclobutyl acetate. To a solution of 1-(3-bromo-6-chlorothieno[2,3-b]pyridin-2-yl)-3,3-difluorocyclobutyl acetate (0.45 g, 1.14 mmol) in 1,4-dioxane (8.10 mL) and H₂O (0.90 mL) was added 2,4,6-trimethyl-1,3,5,2,4,6-trioxatriborinane (4 M in THF, 1.424 g, 5.67 mmol) and cesium carbonate (1.11 g, 3.40 mmol), and the mixture was degassed with N₂ for 5 min. Then, PdCl₂(dppf) (0.083 g, 0.11 mmol) was added, and the reaction mixture was stirred at 80° C. for 4 h. The reaction mixture was diluted with H₂O (25 mL) and extracted with EtOAc (3×25 mL). The combined organic extracts were washed with satd. aq. NaCl (25 mL), dried over Na₂SO₄, filtered and concentrated. The crude material was purified by chromatography, eluting with a gradient of 3%-10% EtOAc in hexane, to provide 1-(6-chloro-3-methylthieno[2,3-b]pyridin-2-yl)-3,3-difluorocyclobutyl acetate (0.15 g, 0.45 mmol, 40% yield). m/z (ESI): 332.0 (M+H)⁺. ¹H NMR (400 MHz, DMSO-d₆) δ. 8.27 (d, J=8.5 Hz, 1H), 7.56 (d, J=8.5 Hz, 1H), 3.47 (t, J=11.8 Hz, 4H), 2.39 (s, 3H), 2.03 (s, 3H).

Step 3: 1-(6-chloro-3-methylthieno[2,3-b]pyridin-2-yl)-3,3-difluorocyclobutan-1-ol. To a solution of 1-(6-chloro-3-methylthieno[2,3-b]pyridin-2-yl)-3,3-difluorocyclobutyl acetate (0.16 g, 0.48 mmol) in MeOH (4.80 mL) and H₂O (0.48 mL) was added K₂CO₃ (200 mg, 1.5 mmol) at rt. The reaction mass was stirred at rt for 1 h. Then, the solvent was concentrated. The residue was diluted with H₂O (25 mL) and extracted with EtOAc (30 mL×3). The combined organic layers were washed with brine (30 mL), dried over Na₂SO₄, and concentrated under reduced pressure to get 1-(6-chloro-3-methylthieno[2,3-b]pyridin-2-yl)-3,3-difluorocyclobutan-1-ol (120 mg, 0.41 mmol, 86% yield). m/z (ESI): 290.0 (M+H)⁺. H NMR (400 MHz, DMSO-d₆) δ 8.24 (d, J=8.4 Hz, 1H), 7.55 (d, J=8.4 Hz, 1H), 6.62 (s, 1H), 3.28 (td, J=12.1, 3.3 Hz, 2H), 3.08 (td, J=14.0, 10.2 Hz, 2H), 2.41 (s, 3H).

Step 4: 3,3-difluoro-1-(3-methyl-6-(2-methyl-2H-pyrazolo[3,4-b]pyridin-5-yl)thieno[2,3-b]pyridin-2-yl)cyclobutan-1-ol. A stirred solution of 1-(6-chloro-3-methylthieno[2,3-b]pyridin-2-yl)-3,3-difluorocyclobutan-1-ol (0.12 g, 0.41 mmol), 2-methyl-5-(4,4,5,5-tetramethyl-1,3,2-dioxaborolan-2-yl)-2H-pyrazolo[3,4-b]pyridine (161 mg, 0.62 mmol) and K₂CO₃ (0.172 g, 1.24 mmol) in 1,4-dioxane (4.32 mL) and H₂O (0.480 mL) was purged with N₂ gas for 5 min. After purging, SPhos Pd G3 (32 mg, 0.04 mmol) was added, and the reaction mixture was stirred at 110° C. for 2 h. Then, the reaction mass was diluted with EtOAc (50 mL) and filtered through celite. The filtrate was washed with satd. aq. NH₄Cl (50 mL), dried over Na₂SO₄, filtered and concentrated. The crude was purified by reverse-phase preparative HPLC, eluting with a linear gradient of 5%-95% ACN in (0.1% NH₃) H₂O and a flow rate of 15 mL/min, to get Compound C77. m/z (ESI): 387.2 (M+H)⁺. ¹H NMR (400 MHz, DMSO-d₆) δ 9.40 (d, J=2.3 Hz, 1H), 8.95 (d, J=2.4 Hz, 1H), 8.55 (s, 1H), 8.27 (d, J=8.5 Hz, 1H), 8.16 (d, J=8.5 Hz, 1H), 6.57 (s, 1H), 4.25 (s, 3H), 3.28 (d, J=13.0 Hz, 2H), 3.11 (td, J=13.9, 10.0 Hz, 2H), 2.44 (s, 3H).

### Group 23

Example 23-1: (R)-(3,3-difluorocyclobutyl)(3-(2-methyl-2H-pyrazolo[3,4-b]pyridin-5-yl)thieno[2,3-b]pyrazin-6-yl)methanol B92R

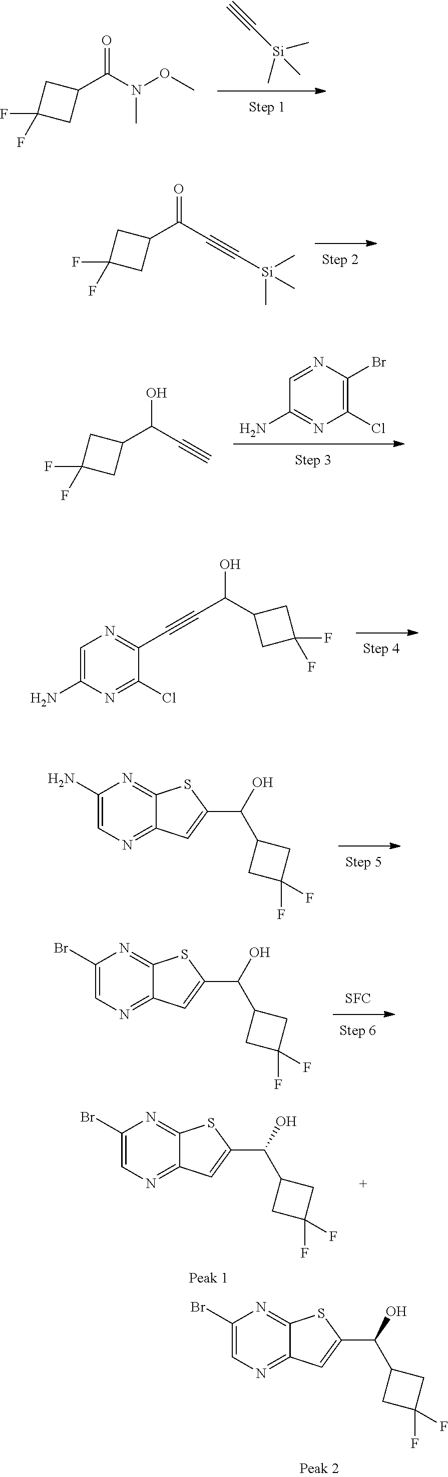

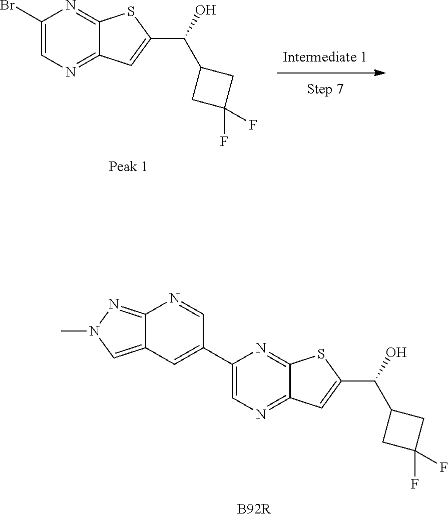 [SMILES: O[C@@H](c1cc2ncc(Br)nc2s1)C1CC(F)(F)C1 ; "B92R" ; Cn1cc2cc(-c3cnc4cc([C@H](O)C5CC(F)(F)C5)sc4n3)cnc2n1]

Step 1: 1-(3,3-difluorocyclobutyl)-3-(trimethylsilyl)prop-2-yn-1-one. To a stirred solution of trimethylsilyl acetylene (6.27 mL, 44.7 mmol) in THF (80 mL) at −78° C. under N₂ gas was added n-butyllithium (2.5 M in hexane, 19.7 mL, 49.1 mmol) dropwise at −78° C., and the mixture was stirred at -78° C. for 30 min. Then, 3,3-difluoro-N-methoxy-N-methylcyclobutane-1-carboxamide (8 g, 44.7 mmol) in THF (20 mL) was added at −78° C., and the reaction was stirred at −78° C. for 3 h. The reaction mixture was quenched by addition of satd. aq. NH₄Cl (100 mL) and extracted with EtOAc (2×250 mL). The combined organic layers were washed with H₂O (100 mL), washed with brine (100 mL), dried over Na₂SO₄, filtered, and concentrated. The crude material was purified by chromatography, eluting with 5% Et₂O in pet. ether, to afford 1-(3,3-difluorocyclobutyl)-3-(trimethylsilyl)prop-2-yn-1-one (6.0 g, 62% yield). ¹H NMR (401 MHz, Chloroform-d) δ 3.18-3.11 (m, 1H), 3.01-2.73 (m, 4H), 0.28 (s, 9H).

Step 2: 1-(3,3-difluorocyclobutyl)prop-2-yn-1-ol. To a stirred solution of 1-(3,3-difluorocyclobutyl)-3-(trimethylsilyl)prop-2-yn-1-one (6 g, 27.7 mmol) in MeOH (60 mL) under N₂ gas at 0° C. was added sodium borohydride (2.099 g, 55.5 mmol), and the reaction was stirred at RT for 1 h. The reaction mixture was quenched by addition of H₂O (60 mL) at 0° C., partially concentrated, and extracted with EtOAc (3×100 mL). The combined organic layers were washed with brine (100 mL), dried over Na₂SO₄, filtered, and concentrated. The crude material was purified by chromatography, eluting with 10% EtOAc in pet. ether, to afford 1-(3,3-difluorocyclobutyl)prop-2-yn-1-ol (3 g, 20.53 mmol, 74% yield). ¹H NMR (401 MHz, DMSO-d₆) δ 5.67 (d, J=5.8 Hz, 1H), 4.25 (td, J=6.0, 2.1 Hz, 1H), 3.33-3.31 (d, J=2.1 Hz, 1H), 2.64-2.28 (m, 5H).

Step 3: 3-(5-amino-3-chloropyrazin-2-yl)-1-(3,3-difluorocyclobutyl)prop-2-yn-1-ol. To a stirred solution of 5-bromo-6-chloropyrazin-2-amine (2 g, 9.6 mmol) in THF (20 mL) under N₂ was added copper(I) iodide (0.091 g, 0.48 mmol), Et₃N (8.01 mL, 57.6 mmol) and Pd(dppf)Cl₂-DCM (0.784 g, 0.96 mmol), and the mixture was purged under N₂ for 5 min. Then, 1-(3,3-difluorocyclobutyl)prop-2-yn-1-ol (1.82 g, 12.5 mmol) in THF (5 mL) was added, and the reaction mixture was stirred at 85° C. for 16 h. After, the reaction mass was cooled to rt and concentrated, followed by addition of H₂O (50 mL) and EtOAc (100 mL). The mixture was filtered through celite and separated. The aqueous layer was extracted with EtOAc (2×50 mL). The combined organic layers were dried over Na₂SO₄, filtered and concentrated. The crude material was purified by chromatography, eluting with a gradient of 40%-50% EtOAc in pet. ether, to afford 3-(5-amino-3-chloropyrazin-2-yl)-1-(3,3-difluorocyclobutyl)prop-2-yn-1-ol (1.6 g). m/z (ESI): 274.2 (M+H)⁺. ¹H NMR (401 MHz, DMSO-d₆) δ 7.80 (s, 1H), 7.32 (s, 2H), 5.85 (d, J=5.7 Hz, 1H), 4.56 (t, J=5.9 Hz, 1H), 2.59-2.42 (m, 5H).

Step 4: (3-aminothieno[2,3-b]pyrazin-6-yl)(3,3-difluorocyclobutyl)methanol. To a stirred solution of 3-(5-amino-3-chloropyrazin-2-yl)-1-(3,3-difluorocyclobutyl)prop-2-yn-1-ol (1.6 g, 5.85 mmol) in DMF (16 mL) under N₂ gas was added sodium sulfide pentahydrate (3.93 g, 23.4 mmol) at rt, and the mixture was stirred at 95° C. for 2 h. Then, the reaction was cooled to rt and H₂O (50 mL) and EtOAc (100 mL) were added. The mixture was filtered through celite and separated. The aqueous layer was extracted with EtOAc (2×50 mL). The combined organic layers were dried over Na₂SO₄, filtered and concentrated. The crude material was purified by chromatography, eluting with 50% EtOAc in pet. ether, to afford (3-aminothieno[2,3-b]pyrazin-6-yl)(3,3-difluorocyclobutyl)methanol (0.8 g). m/z (ESI): 272.0 (M+H)⁺. ¹H NMR (401 MHz, DMSO-d₆) δ 7.94 (d, J=8.6 Hz, 1H), 7.14 (s, 1H), 6.64 (s, 2H), 6.08 (d, J=5.1 Hz, 1H), 4.80 (t, J=5.4 Hz, 1H), 2.62-2.51 (m, 5H).

Step 5: (3-bromothieno[2,3-b]pyrazin-6-yl)(3,3-difluorocyclobutyl)methanol. To a stirred solution of (3-aminothieno[2,3-b]pyrazin-6-yl)(3,3-difluorocyclobutyl)methanol (0.8 g, 3 mmol) in dibromomethane (16 mL) was added isoamyl nitrite (1.19 mL, 8.85 mmol) at 0° C., and the mixture was stirred at rt for 1 h. Bromotrimethylsilane (2.34 mL, 17.7 mmol) was added at 0° C., and the resulting reaction mass was stirred at rt for 20 h. The reaction mixture was cooled to 0° C., quenched by addition of 10% aq. NaHCO₃ and extracted with DCM (2×100 mL). The combined organic layers were washed with brine (50 mL), dried over Na₂SO₄, filtered, and concentrated. The crude material was purified by chromatography, eluting with 5-10% EtOAc in pet. ether, to afford (3-bromothieno[2,3-b]pyrazin-6-yl)(3,3-difluorocyclobutyl)methanol (200 mg). m/z (ESI): 335.0 (M+H)⁺. ¹H NMR (401 MHz, DMSO-d₆) δ 8.86 (s, 1H), 7.54 (s, 1H), 6.52 (d, J=5.2 Hz, 1H), 5.01 (s, 1H), 2.67-2.52 (m, 5H).

Step 6: SFC Separation. The sample of (3-bromothieno[2,3-b]pyrazin-6-yl)(3,3-difluorocyclobutyl)methanol was purified via SFC by using a Chiralpak IG 25×5.0 cm, 5 μm column, eluting with a mobile phase of 40% EtOH in liquid CO₂, using a flow rate of 150 mL/min to provide a 1^{s}eluting peak as (R)-(3-bromothieno[2,3-b]pyrazin-6-yl)(3,3-difluorocyclobutyl)methanol and a 2^{nd} eluting peak as (S)-(3-bromothieno[2,3-b]pyrazin-6-yl)(3,3-difluorocyclobutyl)methanol.

Peak 1. (R)-(3-bromothieno[2,3-b]pyrazin-6-yl)(3,3-difluorocyclobutyl)methanol was isolated as the 1^{st} eluting isomer (90 mg). m/z (ESI): 335.0 (M+H)⁺. ¹H NMR (401 MHz, DMSO-d₆) δ 8.86 (s, 1H), 7.54 (s, 1H), 6.53 (d, J=5.2 Hz, 1H), 5.02-4.99 (m, 1H), 2.67-2.52 (m, 5H).

Peak 2. (S)-(3-bromothieno[2,3-b]pyrazin-6-yl)(3,3-difluorocyclobutyl)methanol was isolated as the 2^{nd} eluting isomer (45 mg). m/z (ESI): 335.0 (M+H)⁺. ¹H NMR (401 MHz, DMSO-d₆) δ 8.86 (s, 1H), 7.54 (s, 1H), 6.52 (d, J=5.2 Hz, 1H), 5.02-4.99 (m, 1H), 2.67-2.51 (m, 5H).

Step 7: (R)-(3,3-difluorocyclobutyl)(3-(2-methyl-2H-pyrazolo[3,4-b]pyridin-5-yl)thieno[2,3-b]pyrazin-6-yl)methanol. A stirred solution of (R)-(3-bromothieno[2,3-b]pyrazin-6-yl)(3,3-difluorocyclobutyl)methanol (90 mg, 0.27 mmol), 2-methyl-5-(4,4,5,5-tetramethyl-1,3,2-dioxaborolan-2-yl)-2H-pyrazolo[3,4-b]pyridine (90 mg, 0.35 mmol) and K₂CO₃ (111 g, 0.81 mmol) in 1,4-dioxane (1.4 mL) and H₂O (0.4 mL) was purged with N₂ gas for 2 min. After purging, SPhos Pd G3 (21 mg, 0.03 mmol) was added, and the reaction mixture was stirred at 110° C. for 2 h. Then, the reaction mass was diluted with EtOAc, filtered through celite, washed with EtOAc (25 mL), and concentrated. The crude material was purified by reverse-phase preparative HPLC, eluting with a linear gradient of 5%-95% ACN in (0.1% NH₃) H₂O with a flow rate of 15 mL/min, to get B92R (70 mg, 67% yield). m/z (ESI): 388.2 (M+H)⁺. ¹H NMR (401 MHz, DMSO-d₆) δ 9.41-9.39 (m, 2H), 9.02 (d, J=2.3 Hz, 1H), 8.60 (s, 1H), 7.54 (s, 1H), 6.47 (s, 1H), 5.03 (d, J=3.5 Hz, 1H), 4.26 (s, 3H), 2.61-2.50 (m, 5H).

Example 23-2: (S)-(3,3-difluorocyclobutyl)(3-(2-methyl-2H-pyrazolo[3,4-b]pyridin-5-yl)thieno[2,3-b]pyrazin-6-yl)methanol B92S

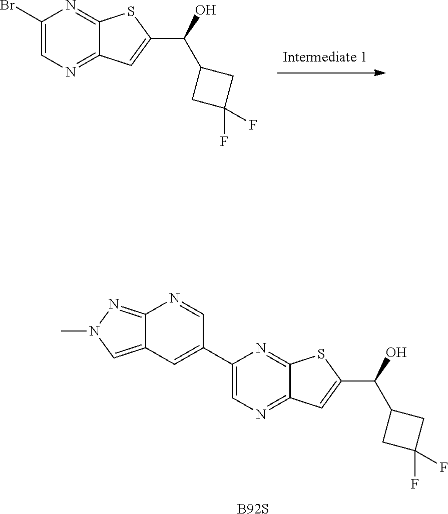 [SMILES: O[C@H](c1cc2ncc(Br)nc2s1)C1CC(F)(F)C1 ; "B92S" ; Cn1cc2cc(-c3cnc4cc([C@@H](O)C5CC(F)(F)C5)sc4n3)cnc2n1]

(S)-(3,3-difluorocyclobutyl)(3-(2-methyl-2H-pyrazolo[3,4-b]pyridin-5-yl)thieno[2,3-b]pyrazin-6-yl)methanol. A stirred solution of (S)-(3-bromothieno[2,3-b]pyrazin-6-yl)(3,3-difluorocyclobutyl)methanol (45 mg, 0.13 mmol), 2-methyl-5-(4,4,5,5-tetramethyl-1,3,2-dioxaborolan-2-yl)-2H-pyrazolo[3,4-b]pyridine (45 mg, 0.18 mmol) and K₂CO₃ (55.7 mg, 0.40 mmol) in 1,4-dioxane (0.72 mL) and H₂O (0.18 mL) was purged with N₂ gas for 2 min. After purging, SPhos Pd G3 (10.5 mg, 0.013 mmol) was added, and the reaction mixture was stirred at 110° C. for 2 h. Then, the reaction was diluted with EtOAc, filtered through celite, washed with EtOAc (15 mL), and concentrated. The crude was purified by reverse-phase preparative HPLC, eluting with a linear gradient of 5%-95% ACN in (0.1% NH₃) H₂O with a flow rate of 15 mL/min, to get B92S (45 mg, 87% yield). m/z (ESI): 388.2 (M+H)⁺. ¹H NMR (401 MHz, DMSO-d₆) δ 9.41-9.39 (m, 2H), 9.02 (d, J=2.3 Hz, 1H), 8.60 (s, 1H), 7.54 (s, 1H), 6.47 (s, 1H), 5.03 (d, J=3.5 Hz, 1H), 4.25 (s, 3H), 2.62-2.50 (m, 5H).

### Alternate Conditions in Reference to Example 23-1

(1) Boc protection: To a stirred mixture of the product of Step 3 (1 equiv) in THF (0.22 M) at rt under N₂ was added triethylamine (3.0 equiv), Boc anhydride (1.2 equiv) and DMAP (0.1 equiv). The reaction mixture was stirred at rt for 16 h. The reaction mixture was quenched with water (25 mL) and extracted with ethyl acetate (2×25 mL). The combined organic layers were washed with water (20 mL), dried over Na2SO4, filtered and concentrated. The resulting crude product was purified by column chromatography, eluting with 5-10% ethyl acetate in pet. ether, to afford a mixture of mono and diBoc product that was used directly in the next step.
(2) Deprotection: To a stirred solution of the product of Step 4 in DCM (0.27 M) at 0° C. under N₂ was added TFA (0.824 mL, 10.77 mmol, 10.0 equiv). The reaction mixture was stirred at rt for 16 h. The reaction mixture was concentrated and quenched with 10% aq. NaHCO₃. The reaction mixture was extracted with DCM (2×25 mL), dried over Na₂SO₄, filtered and concentrated. The resulting crude product was purified by column chromatography, eluting with 30-40% ethyl acetate in pet. ether, to afford product.

The compounds in Table 23-1 were prepared following the procedure described in Group 23, using appropriate starting materials (e.g., aldehyde/ketones and boronate esters). All starting materials are commercially available or are described in the Intermediates section above.

TABLE 23-1

| Ex. # | Chemical Structure & Name | LCMS: (ESI + ve ion) m/z; NMR | Reaction Comments |
|---|---|---|---|
| B96R | 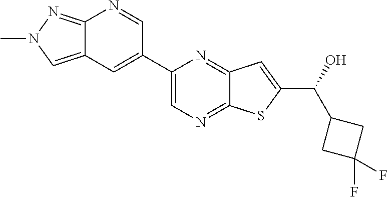 [SMILES: Cn1cc2cc(-c3cnc4sc([C@H](O)C5CC(F)(F)C5)cc4n3)cnc2n1] | 388.2; ¹H NMR (401 MHz, DMSO-d₆) δ 9.39 (d, J = 2.3 Hz, 1H), 9.25 (s, 1H), 9.00 (d, J = 2.3 Hz, 1H), 8.60 (s, 1H), 7.58 (s, 1H), 6.49 (s, 1H), 5.05 (d, J = 3.5 Hz, 1H), 4.26 (s, 3H), 2.68-2.56 (m, 5H). | Alternate conditions (1) and (2) were used |
| | (R)-(3,3-difluorocyclobutyl)(2-(2-methyl-2H-pyrazolo[3,4- | | |
| | b]pyridin-5-yl)thieno[2,3-b]pyrazin-6-yl)methanol | | |
| B96S | 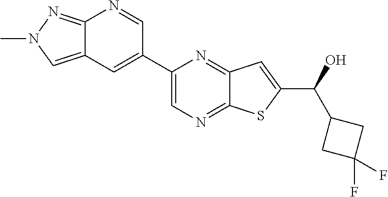 [SMILES: Cn1cc2cc(-c3cnc4sc([C@@H](O)C5CC(F)(F)C5)cc4n3)cnc2n1] | 388.2; ¹H NMR (401 MHz, DMSO-d₆) δ 9.39 (d, J = 2.3 Hz, 1H), 9.25 (s, 1H), 9.00 (d, J = 2.3 Hz, 1H), 8.60 (s, 1H), 7.58 (s, 1H), 6.49 (s, 1H), 5.08-5.03 (m, 1H), 4.26 (s, 3H), 2.67-2.50 (m, 5H). | Alternate conditions (1) and (2) were used |
| | (S)-(3,3-difluorocyclobutyl)(2-(2-methyl-2H-pyrazolo[3,4- | | |
| | b]pyridin-5-yl)thieno[2,3-b]pyrazin-6-yl)methanol | | |

### Group 24

Example 24-1: (2S)-2-(3,3-difluorocyclobutyl)-2-(3-methyl-6-(2-methyl-2H-pyrazolo[3,4-b]pyridin-5-yl)thieno[2,3-b]pyridin-2-yl)ethanol B93S; (2R)-2-(3,3-difluorocyclobutyl)-2-(3-methyl-6-(2-methyl-2H-pyrazolo[3,4-b]pyridin-5-yl)thieno[2,3-b]pyridin-2-yl)ethanol B93R

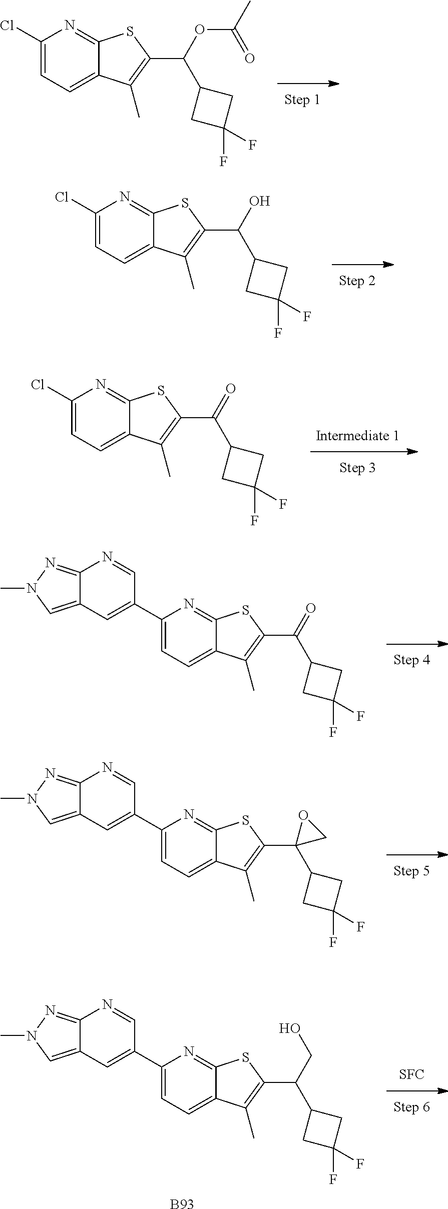

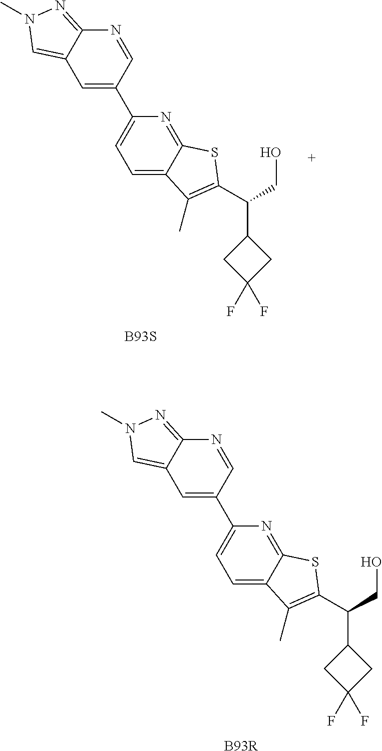 [SMILES: "B93S" ; "B93R" ; Cc1c([C@H](CO)C2CC(F)(F)C2)sc2nc(-c3cnc4nn(C)cc4c3)ccc12 ; Cc1c([C@@H](CO)C2CC(F)(F)C2)sc2nc(-c3cnc4nn(C)cc4c3)ccc12]

Step 1: (6-chloro-3-methylthieno[2,3-b]pyridin-2-yl)(3,3-difluorocyclobutyl)methanol. To a solution of (6-chloro-3-methylthieno[2,3-b]pyridin-2-yl)(3,3-difluorocyclobutyl)methyl acetate (1.9 g, 5.5 mmol) in MeOH (34.2 mL) and H₂O (3.80 mL) was added K₂CO₃ (2.28 g, 16.5 mmol) at rt, and the reaction mass was stirred for 1 h. Then, the solution was concentrated, diluted with H₂O (25 mL), and extracted with EtOAc (30 mL×3). The combined organic layers were washed with brine (30 mL), dried over Na₂SO₄, and concentrated. The crude material was purified by chromatography, eluting with a gradient of 15% to 30% EtOAc in hexane, to provide (6-chloro-3-methylthieno[2,3-b]pyridin-2-yl)(3,3-difluorocyclobutyl)methanol (1.4 g, 4.61 mmol, 84% yield). m/z (ESI): 304.0 (M+H)⁺. ¹H NMR (401 MHz, DMSO-d₆) δ 8.18 (d, J=8.4 Hz, 1H), 7.51 (d, J=8.4 Hz, 1H), 6.23 (d, J=4.4 Hz, 1H), 5.09 (d, J=3.6 Hz, 1H), 2.67 (dd, J=22.0, 9.0 Hz, 1H), 2.49-2.45 (m, 4H), 2.37 (s, 3H).

Step 2: (6-chloro-3-methylthieno[2,3-b]pyridin-2-yl)(3,3-difluorocyclobutyl)methanone. To a solution of (6-chloro-3-methylthieno[2,3-b]pyridin-2-yl)(3,3-difluorocyclobutyl)methanol (1.4 g, 4.6 mmol) in EtOAc (70.0 mL) was added IBX (3.9 g, 13.8 mmol) at rt, and the reaction was stirred at 70° C. for 16 h. Then, the reaction was cooled to rt, diluted with EtOAc (100 mL), filtered through celite, washed with EtOAc (100 mL), and concentrated. The crude material was purified by chromatography, eluting with a gradient of 5% to 12% EtOAc in hexane, to provide (6-chloro-3-methylthieno[2,3-b]pyridin-2-yl)(3,3-difluorocyclobutyl)methanone (1.1 g, 3.7 mmol, 79% yield). m/z (ESI): 302.1 (M+H)⁺. ¹H NMR (401 MHz, DMSO-d₆) δ 8.54 (d, J=8.6 Hz, 1H), 7.67 (d, J=8.6 Hz, 1H), 3.95-3.81 (m, 1H), 3.06-2.90 (m, 4H), 2.74 (s, 3H).

Step 3: (3,3-difluorocyclobutyl)(3-methyl-6-(2-methyl-2H-pyrazolo[3,4-b]pyridin-5-yl)thieno[2,3-b]pyridin-2-yl)methanone. A stirred solution of (6-chloro-3-methylthieno[2,3-b]pyridin-2-yl)(3,3-difluorocyclobutyl)methanone (0.55 g, 1.82 mmol), 2-methyl-5-(4,4,5,5-tetramethyl-1,3,2-dioxaborolan-2-yl)-2H-pyrazolo[3,4-b]pyridine (0.708 g, 2.73 mmol) and K₂CO₃ (0.756 g, 5.47 mmol) in 1,4-dioxane (19.80 mL) and H₂O (2.200 mL) was purged with N₂ gas for 5 min. After purging, SPhos Pd G3 (0.142 g, 0.182 mmol) was added, and the reaction mixture was stirred at 110° C. for 2 h. Then, the reaction mixture was concentrated, diluted with 10% MeOH in DCM (200 mL) and washed with brine (50 mL). The organic layer was dried over Na₂SO₄, filtered, and concentrated. The crude material was slurried in EtOAc (75 mL) and filtered, and the resulting solid was washed with pet. ether (100 mL) to provide (3,3-difluorocyclobutyl)(3-methyl-6-(2-methyl-2H-pyrazolo[3,4-b]pyridin-5-yl)thieno[2,3-b]pyridin-2-yl)methanone (0.55 g, 1.38 mmol, 76% yield). m/z (ESI): 399.0 (M+H)⁺. ¹H NMR (400 MHz, DMSO-d₆) δ 9.44 (d, J=2.4 Hz, 1H), 9.03 (d, J=2.3 Hz, 1H), 8.63-8.53 (m, 2H), 8.27 (d, J=8.7 Hz, 1H), 4.25 (s, 3H), 3.88 (qd, J=8.5, 2.7 Hz, 1H), 3.05-2.89 (m, 4H), 2.77 (s, 3H).

Step 4: 2-(2-(3,3-difluorocyclobutyl)oxiran-2-yl)-3-methyl-6-(2-methyl-2H-pyrazolo[3,4-b]pyridin-5-yl)thieno[2,3-b]pyridine. To a solution of potassium tert-butoxide (0.186 g, 1.656 mmol) in dimethyl sulfoxide (22.00 mL) at rt was added trimethylsulfoxonium iodide (0.365 g, 1.656 mmol), and the mixture was stirred for 30 minutes. A solution of (3,3-difluorocyclobutyl)(3-methyl-6-(2-methyl-2H-pyrazolo[3,4-b]pyridin-5-yl)thieno[2,3-b]pyridin-2-yl)methanone (0.55 g, 1.380 mmol) in dimethyl sulfoxide (22.00 mL) was added, and the reaction mixture was stirred for 5 h. Then, the reaction mixture was diluted with H₂O at 0° C. (50 mL) and extracted with EtOAc (30 mL×2). The combined organic extracts were washed with satd. aq. NaCl (2×50 mL), dried over Na₂SO₄, filtered, and concentrated. The crude material was purified by chromatography, eluting with a gradient of 1%-3% MeOH in DCM, to provide 2-(2-(3,3-difluorocyclobutyl)oxiran-2-yl)-3-methyl-6-(2-methyl-2H-pyrazolo[3,4-b]pyridin-5-yl)thieno[2,3-b]pyridine (360 g, 0.87 mmol, 63% yield). m/z (ESI): 413.2 (M+H)⁺. ¹H NMR (400 MHz, DMSO-d₆) δ 9.39 (d, J=2.3 Hz, 1H), 8.94 (d, J=2.4 Hz, 1H), 8.55 (s, 1H), 8.29 (d, J=8.5 Hz, 1H), 8.16 (d, J=8.5 Hz, 1H), 4.25 (s, 3H), 3.19 (d, J=4.5 Hz, 1H), 3.15 (d, J=4.6 Hz, 1H), 3.05 (tt, J=8.9, 4.4 Hz, 1H), 2.71-2.54 (m, 4H), 2.47 (s, 3H).

Step 5: 2-(3,3-difluorocyclobutyl)-2-(3-methyl-6-(2-methyl-2H-pyrazolo[3,4-b]pyridin-5-yl)thieno[2,3-b]pyridin-2-yl)ethan-1-ol. To a stirred solution of 2-(2-(3,3-difluorocyclobutyl)oxiran-2-yl)-3-methyl-6-(2-methyl-2H-pyrazolo[3,4-b]pyridin-5-yl)thieno[2,3-b]pyridine (0.36 g, 0.87 mmol) in MeOH (7.20 mL) and THF (7.20 mL) was added Pd(OAc)₂ (196 mg, 0.87 mmol), and the mixture was stirred for 5 min. Next, 10 wt % Pd/C (93 mg, 0.087 mmol) was added, and the reaction mixture was stirred at rt for 12 h under an H₂ atmosphere. The reaction was filtered through celite, washed with 20% MeOH in DCM (3×50 mL), and concentrated. The crude was purified by reverse-phase preparative HPLC, eluting with a linear gradient of 5%-95% ACN in (0.1% NH₃) H₂O with a flow rate of 15 mL/min, to get 2-(3,3-difluorocyclobutyl)-2-(3-methyl-6-(2-methyl-2H-pyrazolo[3,4-b]pyridin-5-yl)thieno[2,3-b]pyridin-2-yl)ethan-1-ol (8 mg, 0.019 mmol, 16% yield). m/z (ESI): 415.2 (M+H)⁺.

Step 6: SFC Separation. The stereoisomers were purified via SFC using a Chiralcel OJ-H 25 ×3.0 cm, 5 μm column with a mobile phase of 30% (1:1) MeOH:ACN in liquid CO₂ using a flowrate of 120 mL/min over 9 min to provide a 1^{s} eluting peak as (2S)-2-(3,3-difluorocyclobutyl)-2-(3-methyl-6-(2-methyl-2H-pyrazolo[3,4-b]pyridin-5-yl)thieno[2,3-b]pyridin-2-yl)ethanol and a 2^{nd} eluting isomer as (2R)-2-(3,3-difluorocyclobutyl)-2-(3-methyl-6-(2-methyl-2H-pyrazolo[3,4-b]pyridin-5-yl)thieno[2,3-b]pyridin-2-yl)ethanol.

Peak 1-B93R was isolated as the 1^{st} eluting isomer (36 mg). m/z (ESI): 415.2 (M+H)⁺. ¹H NMR (400 MHz, DMSO-d₆) δ 9.38 (d, J=2.3 Hz, 1H), 8.91 (d, J=2.3 Hz, 1H), 8.54 (s, 1H), 8.21 (d, J=8.4 Hz, 1H), 8.11 (d, J=8.5 Hz, 1H), 4.91 (t, J=5.3 Hz, 1H), 4.24 (s, 3H), 3.64 (dt, J=10.1, 5.3 Hz, 2H), 3.46-3.37 (m, 1H), 2.86-2.71 (m, 1H), 2.59-2.51 (m, 3H), 2.41 (s, 3H), 2.25-2.12 (m, 1H).

Peak 2-B93S was isolated as the 2^{nd} eluting isomer (32 mg). m/z (ESI): 415.2 (M+H)⁺. ¹H NMR (400 MHz, DMSO-d₆) δ 9.38 (d, J=2.1 Hz, 1H), 8.91 (t, J=2.1 Hz, 1H), 8.54 (d, J=1.7 Hz, 1H), 8.21 (d, J=8.5 Hz, 1H), 8.11 (d, J=8.5 Hz, 1H), 4.95-4.88 (m, 1H), 4.24 (s, 3H), 3.65 (dt, J=8.1, 4.0 Hz, 2H), 3.48-3.38 (m, 1H), 2.85-2.72 (m, 1H), 2.59-2.51 (m, 3H), 2.41 (s, 3H), 2.22-2.14 (m, 1H).

### Group 25

Example 25-1: cis-1-(6-(3-methyl-3H-[1,2,3]triazolo[4,5-b]pyridin-6-yl)thieno[2,3-b]pyridin-2-yl)-3-(trifluoromethyl)cyclobutanol C79C

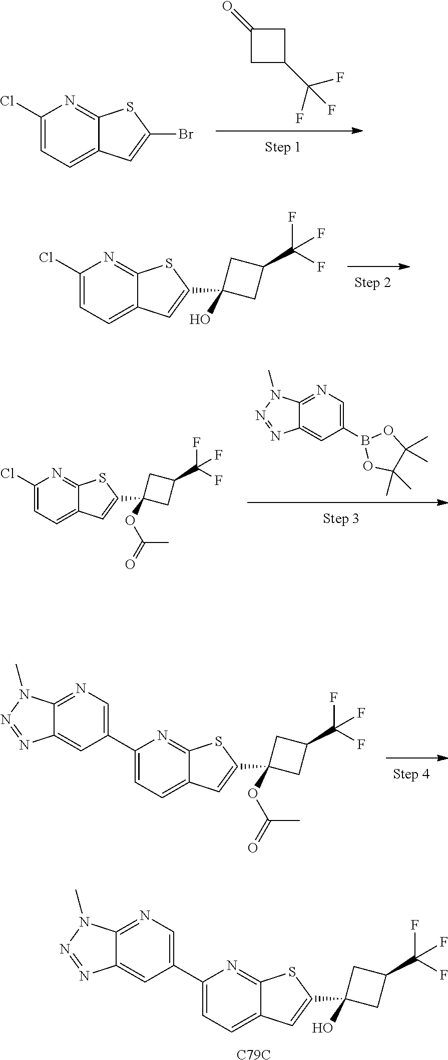

Step 1: cis-1-(6-chlorothieno[2,3-b]pyridin-2-yl)-3-(trifluoromethyl)cyclobutan-1-ol. To a solution of 2-bromo-6-chlorothieno[2,3-b]pyridine (200 mg, 0.8 mmol) and 3-(trifluoromethyl)cyclobutan-1-one (111 mg, 0.81 mmol) in THF (20 mL) at −78° C. was added n-butyllithium (2.5 M in hexane, 0.48 mL, 1.2 mmol) dropwise over 5 min at −78° C., and the reaction mixture was stirred for 1 h at rt. The reaction mixture was quenched by addition of satd. aq. NH₄Cl and extracted with EtOAc (50 mL). The organic layer was washed with H₂O, dried over Na₂SO₄, filtered, and concentrated. The crude material was purified by column chromatography, eluting with a gradient of 10%-15% EtOAc in pet. ether, to afford cis-1-(6-chlorothieno[2,3-b]pyridin-2-yl)-3-(trifluoromethyl)cyclobutan-1-ol (900 mg, 2.9 mmol). m/z (ESI): 308.1 (M+H)⁺. ¹H NMR (400 MHz, DMSO-d₆) δ 8.21 (d, J=8.4 Hz, 1H), 7.58 (s, 1H), 7.51 (d, J=8.4 Hz, 1H), 6.73 (s, 1H), 3.08 (h, J=9.0 Hz, 1H), 2.77-2.66 (m, 2H), 2.55 (dd, J=9.9, 2.9 Hz, 2H).

Step 2: cis-1-(6-chlorothieno[2,3-b]pyridin-2-yl)-3-(trifluoromethyl)cyclobutyl acetate. To a solution of cis-1-(6-chlorothieno[2,3-b]pyridin-2-yl)-3-(trifluoromethyl)cyclobutan-1-ol (700 mg, 2.3 mmol) in DCM (10.0 mL) at 0° C. was added acetic anhydride (1.16 g, 11.4 mmol), N,N-dimethylpyridin-4-amine (28 mg, 0.23 mmol) and Et₃N (0.460 g,4.6 mmol). The reaction mass was warmed to rt and stirred for 2 h, Then, the mixture was concentrated, diluted with H₂O (100 mL), and extracted with DCM (50 mL×3). The combined organic layers were concentrated to give cis-1-(6-chlorothieno[2,3-b]pyridin-2-yl)-3-(trifluoromethyl)cyclobutyl acetate (500 mg, 1.4 mmol, 63% yield). ¹H NMR (401 MHz, DMSO-d₆) δ 8.27 (d, J=8.4 Hz, 1H), 7.73 (s, 1H), 7.56 (d, J=8.4 Hz, 1H), 3.22 (dt, J=17.8, 8.9 Hz, 1H), 3.00 (ddd, J=11.0, 8.4, 2.8 Hz, 2H), 2.74 (td, J=9.9, 2.9 Hz, 2H), 2.06 (s, 3H).

Step 3:cis-1-(6-(3-methyl-3H-[1,2,3]triazolo[4,5-b]pyridin-6-yl)thieno[2,3-b]pyridin-2-yl)-3-(trifluoromethyl)cyclobutyl acetate. To a stirred solution of cis-1-(6-chlorothieno[2,3-b]pyridin-2-yl)-3-(trifluoromethyl)cyclobutyl acetate (100 mg, 0.29 mmol) in 1,4-dioxane (5 mL) and H₂O (1 mL) was added 3-methyl-6-(4,4,5,5-tetramethyl-1,3,2-dioxaborolan-2-yl)-3H-[1,2,3]triazolo[4,5-b]pyridine (112 mg, 0.43 mmol) and K₂CO₃ (119 mg, 0.86 mmol), and the reaction was purged with N₂ for 5 min. After purging, Pd(dppf)Cl₂ (21 mg, 0.03 mmol) was added. The reaction mixture was stirred at 100° C. for 2 h. The reaction mixture was diluted with H₂O (20 mL) and extracted with EtOAc (2×20 mL). The combined organic extracts were washed with satd. aq. NaCl (20 mL), dried over Na₂SO₄, filtered, and concentrated to give crude cis-1-(6-(3-methyl-3H-[1,2,3]triazolo[4,5-b]pyridin-6-yl)thieno[2,3-b]pyridin-2-yl)-3-(trifluoromethyl)cyclobutyl acetate (0.065 g), which was used directly in the next step. m/z (ESI): 448.2 (M+H)⁺.

Step 4: cis-1-(6-(3-methyl-3H-[1,2,3]triazolo[4,5-b]pyridin-6-yl)thieno[2,3-b]pyridin-2-yl)-3-(trifluoromethyl)cyclobutan-1-ol. To a solution of the crude cis-1-(6-(3-methyl-3H-[1,2,3]triazolo[4,5-b]pyridin-6-yl)thieno[2,3-b]pyridin-2-yl)-3-(trifluoromethyl)cyclobutyl acetate (65 mg, 0.16 mmol) in MeOH (10 mL) was added K₂CO₃ (0.185 g, 1.341 mmol), and the reaction mixture was stirred at rt for 2 h. The reaction mixture was concentrated, diluted with H₂O (10 mL), and extracted with EtOAc (2×10 mL). The combined organic extracts were washed with satd. aq. NaCl (10 mL), dried over Na₂SO₄, filtered, and concentrated. The crude material was purified by chromatography, eluting with a gradient of 40% to 50% EtOAc in hexane, to provide cis-1-(6-(3-methyl-3H-[1,2,3]triazolo[4,5-b]pyridin-6-yl)thieno[2,3-b]pyridin-2-yl)-3-(trifluoromethyl)cyclobutan-1-ol (65 mg, 0.16 mmol, 60% yield). m/z (ESI): 406.0 (M+H)⁺. ¹H NMR (401 MHz, DMSO-d₆) δ 9.58 (d, J=2.0 Hz, 1H), 9.23 (d, J=2.0 Hz, 1H), 8.32 (d, J=8.4 Hz, 1H), 8.26 (d, J=8.4 Hz, 1H), 7.60 (s, 1H), 6.73 (s, 1H), 4.37 (s, 3H), 3.11 (dq, J=17.9, 9.0 Hz, 1H), 2.76 (ddd, J=11.0, 8.5, 2.9 Hz, 2H), 2.58 (dd, J=9.9, 2.9 Hz, 2H).

### Group 26

Example 26-1: (S)-(3-amino-6-(2-methyl-2H-pyrazolo[3,4-b]pyridin-5-yl)thieno[2,3-b]pyridin-2-yl)(3,3-difluorocyclobutyl)methanol B95S; (R)-(3-amino-6-(2-methyl-2H-pyrazolo[3,4-b]pyridin-5-yl)thieno[2,3-b]pyridin-2-yl)(3,3-difluorocyclobutyl)methanol B95R

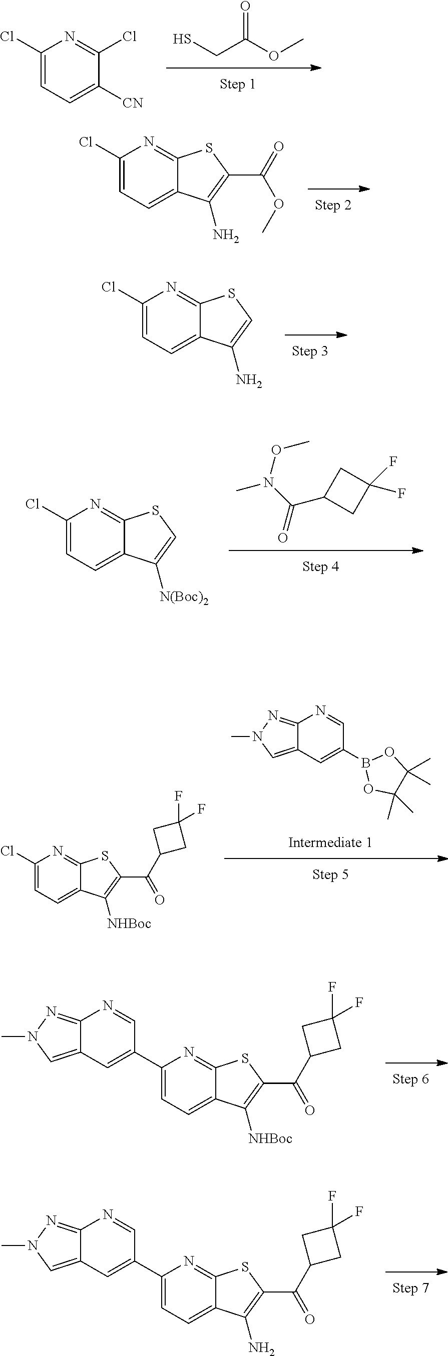

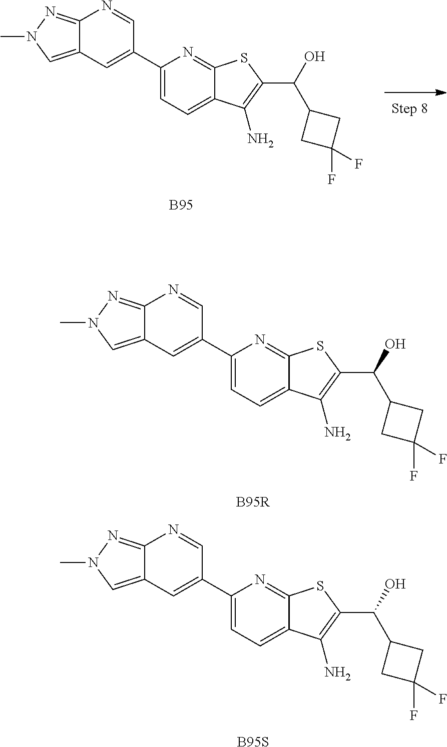 [SMILES: "B95" ; Cn1cc2cc(-c3ccc4c(N)c(C(O)C5CC(F)(F)C5)sc4n3)cnc2n1 ; "B95R" ; "B95S" ; Cn1cc2cc(-c3ccc4c(N)c([C@@H](O)C5CC(F)(F)C5)sc4n3)cnc2n1 ; Cn1cc2cc(-c3ccc4c(N)c([C@H](O)C5CC(F)(F)C5)sc4n3)cnc2n1]

Step 1: methyl 3-amino-6-chlorothieno[2,3-b]pyridine-2-carboxylate. To a solution of 2,6-dichloronicotinonitrile (20 g, 116 mmol) and methyl 2-mercaptoacetate (12.3 g, 116 mmol) in DMF (200 mL) at 0° C. was added KOH (25.9 g, 462 mmol) in water (200 mL). The reaction mixture was stirred at 0° C. for 1 h. The reaction mass was then diluted with water (50 mL) and filtered to provide methyl 3-amino-6-chlorothieno[2,3-b]pyridine-2-carboxylate (6.5 g, 26.8 mmol, 23.2% yield). m/z (ESI). 243.0 (M+H)⁺. ¹H NMR (401 MHz, DMSO-d₆) δ 8.59 (d, J=8.5 Hz, 1H), 7.59 (d, J=8.5 Hz, 1H), 7.37 (s, 2H), 3.81 (s, 3H).

Step 2: 6-chlorothieno[2,3-b]pyridin-3-amine. A solution of methyl 3-amino-6-chlorothieno[2,3-b]pyridine-2-carboxylate (6.5 g, 26.8 mmol) and lithium chloride (5.68 g, 134 mmol) in dimethyl sulfoxide (40 mL) was stirred at 145° C. for 16 h. The reaction mixture was allowed to cool to room temperature. The reaction mixture was then diluted with water (100 mL) and extracted with EtOAc (2×50 mL). The combined organic extracts were washed with satd. aq. NaCl (100 mL), dried over Na2SO4, filtered and concentrated in vacuo to afford crude material. The crude material was purified by reverse phase chromatography to provide 6-chlorothieno[2,3-b]pyridin-3-amine (1.50 g, 8.12 mmol, 30.3% yield). m/z (ESI): 185.0 (M+H)⁺. ¹H NMR (401 MHz, DMSO-d₆) δ 8.27 (d, J=8.4 Hz, 1H), 7.48 (d, J=8.4 Hz, 1H), 6.28 (s, 1H), 5.52 (s, 2H).

Step 3: tert-butyl (tert-butoxycarbonyl)(6-chlorothieno[2,3-b]pyridin-3-yl)carbamate. To a solution of 6-chlorothieno[2,3-b]pyridin-3-amine (1.50 g, 8.12 mmol) in THF (30 mL) was added 4-(dimethylamino)pyridine (0.993 g, 8.12 mmol), Boc-anhydride (2.83 mL, 12.2 mmol), and TEA (2.27 mL, 16.3 mmol). The reaction mixture was stirred for 16 h at rt. The reaction mixture was then diluted with water (100 mL) and extracted with EtOAc (2×50 mL). The combined organic extracts were washed with satd. aq. NaCl (50 mL), dried over Na₂SO₄, filtered and concentrated in vacuo to give crude material. The crude material was purified by reverse phase chromatography to afford tert-butyl (tert-butoxycarbonyl)(6-chlorothieno[2,3-b]pyridin-3-yl)carbamate (1.5 g, 3.90 mmol, 48.0% yield). m z (ESI): 385.0 (M+H)⁺. ¹H NMR (401 MHz, DMSO-d₆) δ 8.05 (dd, J=8.4, 0.9 Hz, 1H), 8.00 (d, J=0.9 Hz, 1H), 7.59 (dd, J=8.4, 0.9 Hz, 1H), 1.36 (s, 18H).

Step 4: tert-butyl (6-chloro-2-(3,3-difluorocyclobutane-1-carbonyl)thieno[2,3-b]pyridin-3-yl)carbamate. To a −78° C. solution of tert-butyl (tert-butoxycarbonyl)(6-chlorothieno[2,3-b]pyridin-3-yl)carbamate (1.4 g, 3.64 mmol) in THF (10 mL) was added a solution of LDA in THF (2.0 M, 2.73 mL, 5.46 mmol). The reaction mixture was stirred at −78° C. for 30 min before 3,3-difluoro-N-methoxy-N— methylcyclobutane-1-carboxamide (1.30 g, 7.28 mmol) was added. The reaction mixture was stirred at -78° C. for 1 h before being quenched with sat. aq. NH₄Cl (50 mL) and extracted with EtOAc (2×50 mL). The combined organic extracts were filtered and concentrated in vacuo to afford crude material. The crude material was purified by reverse phase chromatography to provide tert-butyl (6-chloro-2-(3,3-difluorocyclobutane-1-carbonyl)thieno[2,3-b]pyridin-3-yl)carbamate (800 mg, 1.192 mmol, 32.8% yield). m/z (ESI): 403.0 (M+H)⁺.

Step 5: tert-butyl (2-(3,3-difluorocyclobutane-1-carbonyl)-6-(2-methyl-2H-pyrazolo[3,4-b]pyridin-5-yl)thieno[2,3-b]pyridin-3-yl)carbamate. A mixture of tert-butyl (6-chloro-2-(3,3-difluorocyclobutane-1-carbonyl)thieno[2,3-b]pyridin-3-yl)carbamate (400 mg, 0.993 mmol), 2-methyl-5-(4,4,5,5-tetramethyl-1,3,2-dioxaborolan-2-yl)-2H-pyrazolo[3,4-b]pyridine (334 mg, 1.291 mmol), and potassium carbonate (412 mg, 2.98 mmol) in 1,4-dioxane (10 mL) and water (3.0 mL) was purged with N₂ for 5 min. SPhos Pd G3 (77 mg, 0.099 mmol) was added, and the reaction mixture was stirred at 100° C. for 2 h. The reaction mixture was diluted with water (10 mL) and extracted with EtOAc (2×20 mL). The combined organic extracts were washed with satd. aq. NaCl (20 mL), dried over Na₂SO₄, filtered and concentrated in vacuo to give crude tert-butyl (2-(3,3-difluorocyclobutane-1-carbonyl)-6-(2-methyl-2H-pyrazolo[3,4-b]pyridin-5-yl)thieno[2,3-b]pyridin-3-yl)carbamate, which was used directly in the next step (480 mg). m/z (ESI): 500.2 (M+H)⁺.

Step 6: (3-amino-6-(2-methyl-2H-pyrazolo[3,4-b]pyridin-5-yl)thieno[2,3-b]pyridin-2-yl)(3,3-difluorocyclobutyl)methanone. To a solution of tert-butyl (2-(3,3-difluorocyclobutane-1-carbonyl)-6-(2-methyl-2H-pyrazolo[3,4-b]pyridin-5-yl)thieno[2,3-b]pyridin-3-yl)carbamate (0.400 mmol) in dioxane (10 mL) was added a solution of hydrochloric acid in 1,4-dioxane (4 N, 500 μL, 2.002 mmol). The reaction mixture was stirred at rt for 1 h. The mixture was concentrated in vacuo to afford crude material. The crude material was purified by reverse-phase preparative HPLC to provide (3-amino-6-(2-methyl-2H-pyrazolo[3,4-b]pyridin-5-yl)thieno[2,3-b]pyridin-2-yl)(3,3-difluorocyclobutyl)methanone (150 mg, 0.376 mmol, 94.0% yield). m/z (ESI): 400.0 (M+H)⁺. ¹H NMR (401 MHz, DMSO-d₆) δ 9.41 (d, J=2.3 Hz, 1H), 9.01 (d, J=2.3 Hz, 1H), 8.71 (d, J=8.6 Hz, 1H), 8.60 (s, 1H), 8.23-8.18 (m, 3H), 4.25 (s, 3H), 3.53-3.46 (m, 1H), 2.98-2.83 (m, 4H).

Step 7: 6-(2-methyl-2H-pyrazolo[3,4-b]pyridin-5-yl)thieno[2,3-b]pyridin-2-yl)(3,3-difluorocyclobutyl)methanol. To a solution of (3-amino-6-(2-methyl-2H-pyrazolo[3,4-b]pyridin-5-yl)thieno[2,3-b]pyridin-2-yl)(3,3-difluorocyclobutyl)methanone (150 mg, 0.376 mmol) in methanol (5 mL) and tetrahydrofuran (5 mL) was added sodium borohydride (14.2 mg, 0.376 mmol). The reaction mixture was stirred at rt for 3 h, at which time it was diluted with water (10 mL) and extracted with EtOAc (2×10 mL). The combined organic extracts were washed with satd. aq. NaCl (10 mL), dried over Na₂SO₄, filtered and concentrated in vacuo to give the crude material. The crude material was purified by reverse-phase preparative HPLC to provide (3-amino-6-(2-methyl-2H-pyrazolo[3,4-b]pyridin-5-yl)thieno[2,3-b]pyridin-2-yl)(3,3-difluorocyclobutyl)methanol (120 mg, 0.299 mmol, 80% yield). m z (ESI): 402.2 (M+H)⁺. ¹H NMR (401 MHz, DMSO-d₆) δ 9.37 (d, J=2.3 Hz, 1H), 8.90 (d, J=2.3 Hz, 1H), 8.53 (s, 1H), 8.28 (d, J=8.5 Hz, 1H), 8.06 (d, J=8.5 Hz, 1H), 5.85 (d, J=4.3 Hz, 1H), 5.39 (s, 2H), 5.10 (d, J=4.7 Hz, 1H), 4.24 (s, 3H), 2.75-2.42 (m, 5H).

Step 8: SFC Purification. The sample was purified via SFC using a Chiral Pak IB N-5250×30 mm, 5 column with a mobile phase of (1:1) EtOH:IPA in CO₂ with a flow rate of 140 mL/min to generate peak 1 and peak 2.

Peak 1: B95R. (20 mg, 0.050 mmol, 15.38% yield). m/z (ESI): 402.2 (M+H)⁺. ¹H NMR (401 MHz, DMSO-d₆) δ 9.37 (d, J=2.3 Hz, 1H), 8.90 (d, J=2.3 Hz, 1H), 8.53 (s, 1H), 8.28 (d, J=8.5 Hz, 1H), 8.06 (d, J=8.5 Hz, 1H), 5.87 (br s, 1H), 5.39 (s, 2H), 5.10 (d, J=4.6 Hz, 1H), 4.24 (s, 3H), 2.72-2.40 (m, 5H).

Peak 2: B95S. (11 mg, 0.027 mmol, 8.46% yield). m/z (ESI): 402.2 (M+H)⁺. ¹H NMR (401 MHz, DMSO-d₆) δ 9.37 (d, J=2.3 Hz, 1H), 8.90 (d, J=2.3 Hz, 1H), 8.54 (s, 1H), 8.28 (d, J=8.5 Hz, 1H), 8.06 (d, J=8.5 Hz, 1H), 5.91 (br s, 1H), 5.39 (s, 2H), 5.10 (d, J=4.6 Hz, 1H), 4.24 (s, 3H) 2.72-2.42 (m, 5H).

### Section 3: Biochemical & Cellular Assays

Example 27: Inhibition Study

The compound dose-response of its inhibition of 15-PGDH activity was determined in human recombinant 15-PGDH enzymatical assays. These assays measured 15-PGDH enzymatic activity by quantifying the amount of NADH produced during a 60-minute reaction initiated by the addition of PGE2 and NAD+. Under the conditions of these experiments, the increase of NADH signal upon oxidation of PGE2 and reduction of NAD+ was linearly proportional to 15-PGDH enzymatic activity. The recombinant human, mouse, rat and dog 15-PGDH enzymatic assays were performed in a 25 μL volume of reaction buffer containing 50 mM Tris, pH 7.5, 0.01% Tween-20 and 100 pM DTT in a 384-well microtiter plate. In concentration-response experiments with tested compounds, 22 concentrations from 2-fold serial dilutions in DMSO were pre-incubated with 15-PGDH for 15 minutes at room temperature. Then, PGE2 and β-NAD+ were added to initiate the 15-PGDH reaction. After 60 minutes at room temperature, the reaction was quenched and NADH signal was measured using a microtiter plate reader.

The following data (Table 27-1) provides the IC50 in μM of each compound for inhibiting enzymatic activity of the recombinant 15-PGDH in the assay described above.

TABLE 27-1

| ID | PGDH-Lumi IC₅₀ (μM) |
|---|---|
| A3 | 0.671 |
| A4 | >100.0 |
| B1 | 0.0586 |
| B2 | 0.00151 |
| B3 | 0.0643 |
| B4 | 0.0397 |
| C23 | 5.43E−4 |
| C24 | 0.23 |
| B5 | 0.0784 |
| B6 | 0.00293 |
| B7 | 0.0149 |
| B8 | 3.93E−4 |
| A2 | 2.16 |
| B9 | 17.7 |
| B10 | 0.139 |
| B11 | 0.00712 |
| B12 | 0.0578 |
| B13 | 9.01E−4 |
| B6R | 0.00184 |
| B6S | 0.0896 |
| B15 | 7.01E−4 |
| B16 | 4.71E−4 |
| B11S | 0.0981 |
| B11R | 0.00342 |
| B13R | 5.70E−4 |
| B13S | 0.0236 |
| B18 | 3.82E−4 |
| C20 | 0.0860 |
| C21 | 0.00176 |
| C22 | 0.257 |
| B19 | 5.38E−4 |
| B15S | 0.150 |
| B15R | 4.01E−4 |
| B19RR | 2.44E−4 |
| B19SR | 0.00187 |
| C18 | 0.0129 |
| C19 | 0.0406 |
| B22 | 5.13E−4 |
| A1 | 0.159 |
| B23 | 3.41E−4 |
| B24 | 5.17E−4 |
| B25 | 0.00585 |
| B19(RS)S | 6.37E−4 |
| B27 | 4.02E−4 |
| B28 | 4.63E−4 |
| B29 | 3.80E−4 |
| C15 | 0.00285 |
| C16 | 0.235 |
| C17 | 1.265 |
| B30 | 2.60E−4 |
| B31 | 4.20E−4 |
| C12 | 0.00727 |
| C13 | 0.761 |
| C14 | 0.555 |
| B32 | 0.00118 |
| C8 | 0.0677 |
| C9 | 0.419 |
| C10 | 0.195 |
| C11 | 0.101 |
| C7 | 2.92 |
| C6 | 0.544 |
| C5 | 3.71E−4 |
| C4 | 1.80 |
| B33 | 0.0270 |
| B34S | 4.7 |
| B34R | 0.0124 |
| B34 | 0.0207 |
| B35S | 0.149 |
| B35R | 0.00109 |
| C3 | 0.807 |
| B37S | 1.88 |
| B37R | 0.00874 |
| B38S | 1.01 |
| B38R | 0.093 |
| C2 | 0.225 |
| B39S | 0.184 |
| B39R | 4.23E−4 |
| B40S | 3.4 |
| B40R | 0.00716 |
| C1 | 0.0438 |
| B41R | 9.55E−4 |
| B41S | 0.151 |
| B42 | 0.0128 |
| B43S | 0.626 |
| B43R | 0.0138 |
| B44S | 0.133 |
| B44R | 4.64E−4 |
| B45 | 0.00864 |
| B46S | 0.00210 |
| B46R | 2.77E−4 |
| B47S | 7.58E−4 |
| B47R | 2.098E−4 |
| B48S | 0.0118 |
| B48R | 4.00E−4 |
| B49R | 2.60E−4 |
| B49S | 0.00930 |
| B50S | 0.00186 |
| B50R | 3.43E−4 |
| B51S | 0.00658 |
| B51R | 1.64E−4 |
| B52S | 0.00160 |
| B52R | 2.91E−4 |
| B53S | 4.36E−4 |
| B53R | 2.22E−4 |
| B54S | 0.757 |
| B54R | 0.00411 |
| B54 | 0.00935 |
| B55S | 0.00264 |
| B55R | 3.08E−4 |
| B56S | 0.0183 |
| B56R | 6.03E−4 |
| B56 | 0.00115 |
| B57S | 0.0132 |
| B57R | 3.20E−4 |
| B58S | 0.111 |
| B58R | 2.40E−4 |
| B58 | 4.88E−4 |
| B59S | 1.10 |
| B59 | 0.00841 |
| B59R | 0.00475 |
| B60S | 0.308 |
| B60 | 0.00303 |
| B60R | 0.00117 |
| B61S | 0.0606 |
| B61 | 4.0E−4 |
| B61R | 2.25E−4 |
| B62 | 0.0207 |
| B63 | 0.015 |
| B64S | 5.97E−4 |
| B64R | 3.25E−4 |
| B65S | 0.0231 |
| B65R | 3.81E−4 |
| B65 | 9.44E−4 |
| B66 | 9.48E−4 |
| B66S | 0.155 |
| B66R | 3.96E−4 |
| B30S | 3.50E−4 |
| B30R | 1.59E−4 |
| B49 | 6.08E−4 |
| B31S | 0.00564 |
| B31R | 2.03E−4 |
| B24R | 2.58E−4 |
| B24S | 0.0159 |
| B27S | 0.0123 |
| B27R | 2.55E−4 |
| B29S | 0.0105 |
| B29R | 2.09E−4 |
| B71 | 7.49E−4 |
| B16S | 0.027 |
| B16R | 3.25E−4 |
| B28S | 0.0122 |
| B28R | 4.24E−4 |
| B23S | 0.121 |
| B23R | 4.22E−4 |
| C25 | 3.32E−4 |
| C26 | 2.73E−4 |
| C27 | 3.79E−4 |
| C28 | 4.72E−4 |
| C29C | 3.00E−4 |
| C30C | 2.78E−4 |
| C31C | 3.56E−4 |
| B72S | 0.00958 |
| B72R | 2.16E−4 |
| C32 | 3.69E−4 |
| B73S | 7.91E−4 |
| B73R | 4.12E−4 |
| C33 | 0.00255 |
| C34 | 7.8E−4 |
| C35 | 8.32E−4 |
| C36 | 7.56E−4 |
| C37 | 0.00901 |
| C38 | 0.00459 |
| B74R | 0.00476 |
| B74S | 0.0241 |
| C39 | 0.01 |
| C40T | 0.00658 |
| B75R | 3.82E−4 |
| B75S | 0.0254 |
| B76R | 3.87E−4 |
| B76S | 7.18E−4 |
| C41 | 0.00124 |
| C42 | 0.0238 |
| B77R | 0.00493 |
| B77S | 0.154 |
| B78R | 0.017 |
| B78S | 0.896 |
| C43 | 0.0053 |
| B79R | 3.99E−4 |
| B79S | 7.06E−4 |
| C44 | 0.00756 |
| C45 | 0.00168 |
| B80R | 0.0467 |
| B80S | 0.00116 |
| C46 | 0.0261 |
| C47 | 9.96E−4 |
| D1S | 0.00117 |
| D1R | 6.05E−4 |
| C48 | 0.0277 |
| C49 | 0.0132 |
| C50 | 0.0347 |
| C51 | 0.00219 |
| C52 | 3.61E−4 |
| C53 | 3.63E−4 |
| C54 | 0.00351 |
| C55 | 8.21E−4 |
| B82S | 0.00165 |
| B82R | 3.98E−4 |
| C56T | 0.00118 |
| C56C | 4.23E−4 |
| C57 | 6.2E−4 |
| C58C | 8.89E−4 |
| C59T | 7.27E−4 |
| C59C | 5.84E−4 |
| B83S | 0.00513 |
| B83R | 2.96E−4 |
| C60 | 0.0521 |
| C61 | 2.8E−4 |
| C62C | 3.66E−4 |
| C63T | 3.76E−4 |
| C63C | 4.29E−4 |
| C64SR | 0.466 |
| C64RS | 0.0209 |
| C65S | 1.99 |
| C65R | 0.0326 |
| C66T | 4.7E−4 |
| C66C | 8.52E−4 |
| C67C | 5.34E−4 |
| C68 | 5.8E−4 |
| C69C | 0.00116 |
| C70RR | 0.403 |
| C70RS | 3.86 |
| C70SR | 2.26 |
| C71C | 5.4E−4 |
| C70SS | 2.28 |
| C73 | 0.00244 |
| C74C | 4.58E−4 |
| B84RC | 4.7E−4 |
| B84SC | 0.00153 |
| B84RT | 3.65E−4 |
| B84ST | 0.00121 |
| C75C | 7.07E−4 |
| B85SS | 0.00332 |
| B85RR | 4.7E−4 |
| B85RS | 3.79E−4 |
| B85SR | 0.00122 |
| B86S | 0.286 |
| B86R | 5.04E−4 |
| B87R | 4.94E−4 |
| B87S | 0.0288 |
| B88RC | 5.05E−4 |
| C76C | 8.3167E−4 |
| B88SC | 0.0101 |
| B88RT | 5.24E−4 |
| B88ST | 0.00112 |
| D2 | 0.537 |
| C77 | 0.00769 |
| C78C | 0.00103 |
| C79C | 5.85E−4 |
| C80C | 7.7067E−4 |
| D3R | 21.7 |
| D3S | 11.5 |
| D4S | 0.426 |
| D4R | 0.243 |
| C81C | 6.4133E−4 |
| C82C | 0.00949 |
| B89S | 0.922 |
| B89R | 0.00177 |
| B90S | 0.488 |
| B90R | 9.52E−4 |
| D5C | 0.198 |
| B91S | 0.0349 |
| B91R | 5.44E−4 |
| C40C | 7.42E−4 |
| C83C | 0.00533 |
| D6C | 1.03 |
| C84C | 0.00779 |
| C85C | 0.00289 |
| C86C | 0.00152 |
| D7R | 0.0247 |
| D7S | 0.265 |
| C87C | 6.1033E−4 |
| C88T | 3.47E−4 |
| C88C | 5.03E−4 |
| C89C | 0.0338 |
| C90C | 0.0144 |
| C91T | 0.00214 |
| C91C | 7.99E−4 |
| C92C | 0.00146 |
| C93C | 5.87E−4 |
| B92R | 5.11E−4 |
| B92S | 0.00514 |
| B93R | 0.174 |
| B93S | 0.00264 |
| C94C | 0.002 |
| C95C | 0.00126 |
| C96C | 7.55E−4 |
| C96T | 4.45E−4 |
| C97C | 3.33E−4 |
| C98T | 0.00173 |
| C98C | 0.00869 |
| C99C | 2.91E−4 |
| C100C | 3.0E−4 |
| C101C | 4.96E−4 |
| C102C | 9.71E−4 |
| C103C | 3.02E−4 |
| C104T | 5.15E−4 |
| C105C | 2.94E−4 |
| C106T | 0.0242 |
| C106C | 0.0167 |
| C107 | 4.9E−4 |
| C108 | 6.71E−4 |
| B94SR | 6.4E−4 |
| B94RS | 3.25E−4 |
| B94SS | 0.00123 |
| B94RR | 3.4E−4 |
| C109C | 0.00335 |
| C104C | 0.00388 |
| C111C | 0.00109 |
| C112C | 5.89E−4 |
| C113T | 4.26E−4 |
| C113C | 0.00142 |
| C114T | 4.88E−4 |
| C114C | 3.23E−4 |
| C115T | 5.13E−4 |
| C115C | 3.93E−4 |
| C116 | 4.3E−4 |
| C117C | 4.29E−4 |
| C118T | 8.64E−4 |
| C118C | 5.57E−4 |
| C119C | 0.0028 |
| C120C | 0.101 |
| B95R | 0.00121 |
| B95S | 0.00643 |
| C121T | 0.00652 |
| C121C | 0.0104 |
| C122C | 0.00683 |
| C123C | 9.67E−4 |
| C124C | 0.00201 |
| C125T | 7.5E−4 |
| C125C | 8.17E−4 |
| C102T | 0.00165 |
| C126C | 0.0116 |
| C127C | 0.0064 |
| C128C | 6.20E−04 |
| C129C | 0.0035 |
| B96R | 4.40E−04 |
| B96S | 0.0031 |

Example 28: Cellular 15-PGDH Binding Study

This assay was used for the quantification of IL-13 induced Native Prostaglandin E2 (PGE2) produced by A549 cells (epithelial cells from lung carcinoma) in cell culture supernatants. Inhibition of 15-PGDH blocks PGE2 degradation and resulted in PGE2 accumulation over basal level. Briefly, A549 cells were treated with 2.5 ng/mL IL-10 (EC₅₀) and a 15-PGDH inhibitor for 24 hrs. PGE2 was detected in a competitive HTRF assay by using anti PGE2 antibody labeled with Europium cryptate, and PGE2 labeled with deuterium. The PGE2 present in the sample competes with the binding between the two HTRF detection solutions (reagents) and thereby prevents FRET from occurring. The specific signal is inversely proportional to the PGE2 concentration. From this binding data, IC50 values were calculated.

The following data (Table 28-1) provides the IC50 in μM for each compound for inhibiting enzymatic activity of the recombinant (native or cellular) 15-PGDH in the cellular assay.

TABLE 28-1

| ID | A549 Cell Assay IC50 (μM) |
|---|---|
| B2 | 0.00104 |
| C23 | 0.00477 |
| B6 | 0.0394 |
| B8 | 9.19E−4 |
| B11 | 0.0319 |
| B13 | 0.00419 |
| B6R | 0.00486 |
| B6S | 0.102 |
| B15 | 9.57E−4 |
| B16 | 0.00119 |
| B11R | 0.0269 |
| B13R | 0.0032 |
| B18 | 9.18E−5 |
| C21 | 0.0189 |
| B19 | 1.71E−4 |
| B15S | 4.99E−4 |
| B15R | 5.61E−4 |
| B19RR | 3.81E−4 |
| B19SR | 0.00353 |
| B22 | 0.00129 |
| B23 | 0.00141 |
| B24 | 5.79E−4 |
| B25 | 0.0164 |
| B19(RS)S | 0.00103 |
| B27 | 5.97E−4 |
| B28 | 0.00107 |
| B29 | 0.00104 |
| C15 | 0.0128 |
| B30 | 7.95E−4 |
| B31 | 5.53E−4 |
| B32 | 0.00749 |
| C5 | 0.00169 |
| B34R | 0.0398 |
| B35R | 0.0182 |
| B37R | 0.0617 |
| B39R | 0.00237 |
| B41R | 0.00216 |
| B43R | 0.196 |
| B44R | 0.00261 |
| B46S | 0.01735 |
| B46R | 6.97E−4 |
| B47S | 0.00193 |
| B47R | 3.66E−4 |
| B48R | 0.00177 |
| B49R | 7.23E−4 |
| B50S | 0.00799 |
| B50R | 5.20E−4 |
| B51R | 3.57E−4 |
| B52S | 0.0117 |
| B52R | 6.9E−4 |
| B53S | 2.88E−4 |
| B53R | 3.54E−4 |
| B54R | 0.0118 |
| B55S | 0.0118 |
| B55R | 6.57E−4 |
| B56R | 0.00247 |
| B56 | 0.00322 |
| B57R | 6.57E−4 |
| B58R | 0.00141 |
| B58 | 0.00250 |
| B59R | 0.00779 |
| B60 | 0.00936 |
| B60R | 0.00338 |
| B61 | 0.00180 |
| B61R | 6.97E−4 |
| B64S | 4.83E−4 |
| B64R | 3.11E−4 |
| B65R | 2.63E−4 |
| B65 | 4.37E−4 |
| B66 | 8.29E−4 |
| B66R | 3.03E−4 |
| B30S | 4.06E−4 |
| B30R | 2.33E−4 |
| B49 | 7.39E−4 |
| B31R | 5.3E−4 |
| B24R | 6.14E−4 |
| B27S | 0.00178 |
| B27R | 7.28E−4 |
| B29S | 0.00308 |
| B29R | 7.45E−4 |
| B16S | 0.00958 |
| B16R | 1.49E−4 |
| B28S | 0.00956 |
| B28R | 1.6E−4 |
| B23S | 0.0192 |
| B23R | 3.89E−4 |
| C25 | 1.4E−4 |
| C26 | 6.01E−5 |
| C27 | 5.82E−5 |
| C28 | 4.52E−4 |
| C29C | 5.13E−5 |
| C30C | 1.37E−4 |
| C31C | 1.57E−4 |
| B72S | 0.00527 |
| B72R | 4.79E−5 |
| C32 | 2.55E−4 |
| B73S | 8.63E−4 |
| B73R | 6.1E−5 |
| C33 | 0.00108 |
| C34 | 0.00135 |
| C35 | 0.00135 |
| C36 | 3.48E−4 |
| C37 | 0.00228 |
| C38 | 0.00375 |
| B74R | 4.94E−4 |
| B74S | 0.00448 |
| C39 | 0.00287 |
| C40T | 0.00336 |
| B75R | 1.62E−5 |
| B75S | 0.0125 |
| B76R | 1.26E−5 |
| B76S | 7.25E−4 |
| C41 | 0.00215 |
| C42 | 0.00681 |
| B77R | 0.00129 |
| B77S | 0.0114 |
| B78R | 0.00249 |
| B78S | 0.0571 |
| C43 | 0.00634 |
| B79R | 9.4E−6 |
| B79S | 3.7E−4 |
| C44 | 0.00617 |
| C45 | 0.00135 |
| B80S | 3.39E−4 |
| C46 | 0.0123 |
| C47 | 3.73E−4 |
| D1S | 6.56E−4 |
| D1R | 8.22E−5 |
| C48 | 0.00736 |
| C49 | 0.00182 |
| C50 | 0.0111 |
| C51 | 0.0011 |
| C52 | 9.38E−5 |
| C53 | 3.61E−5 |
| C54 | 0.00306 |
| C55 | 0.00159 |
| B82R | 1.24E−5 |
| C56T | 9.97E−4 |
| C56C | 1.8E−4 |
| C57 | 1.43E−4 |
| C58C | 5.19E−4 |
| C59T | 1.6E−4 |
| C59C | 1.13E−4 |
| B83S | 0.00111 |
| B83R | 1.03E−5 |
| C60 | 0.0267 |
| C61 | 5.94E−5 |
| C62C | 3.21E−5 |
| C63T | 8.63E−5 |
| C63C | 1.15E−4 |
| C64RS | 0.00325 |
| C65R | 0.00345 |
| C66T | 2.23E−5 |
| C66C | 2.06E−4 |
| C67C | 1.87E−5 |
| C68 | 3.61E−4 |
| C69C | 7.22E−4 |
| C70RR | 0.117 |
| C70RS | >0.75 |
| C70SR | 0.172 |
| C71C | 1.42E−4 |
| C70SS | 0.316 |
| C73 | 0.00126 |
| C74C | 1.92E−4 |
| B84RC | 4.92E−4 |
| B84SC | 0.00145 |
| B84RT | 5.15E−4 |
| B84ST | 6.42E−4 |
| C75C | 0.00107 |
| B85SS | 0.00283 |
| B85RR | 7.04E−4 |
| B85RS | 5.17E−4 |
| B85SR | 0.0015 |
| B86S | 0.00161 |
| B86R | 4.99E−4 |
| B87R | 6.67E−4 |
| B87S | 5.72E−4 |
| B88RC | 4.38E−4 |
| C76C | 0.00103 |
| B88SC | 5.22E−4 |
| B88RT | 5.11E−4 |
| B88ST | 5.04E−4 |
| D2 | 0.268 |
| C77 | 0.00915 |
| C78C | 0.00242 |
| C79C | 8.32E−4 |
| C80C | 0.00126 |
| D3R | 0.289 |
| D3S | >0.75 |
| D4S | 0.305 |
| D4R | 0.187 |
| C81C | 0.00269 |
| C82C | 0.0342 |
| B89S | 0.375 |
| B89R | 0.00482 |
| B90S | 0.237 |
| B90R | 8.57E−4 |
| D5C | 0.253 |
| B91S | 0.00237 |
| B91R | 0.00109 |
| C40C | 0.00122 |
| C83C | 0.0123 |
| D6C | 0.354 |
| C84C | 0.0132 |
| C85C | 0.0076 |
| C86C | 0.00412 |
| D7R | 0.0618 |
| D7S | 0.168 |
| C87C | 8.42E−4 |
| C88T | 5.89E−4 |
| C88C | 4.55E−4 |
| C89C | 0.0491 |
| C90C | 0.033 |
| C91T | 0.00585 |
| C91C | 0.00222 |
| C92C | 0.00527 |
| C93C | 0.00196 |
| B92R | 4.09E−4 |
| B92S | 0.00344 |
| B93R | 0.245 |
| B93S | 0.0142 |
| C94C | 0.00145 |
| C95C | 0.00976 |
| C96C | 0.00233 |
| C96T | 0.00139 |
| C97C | 0.00146 |
| C98T | 0.00266 |
| C98C | 0.0187 |
| C99C | 6.66E−4 |
| C100C | 6.71E−4 |
| C101C | 8.47E−4 |
| C102C | 0.00253 |
| C103C | 0.00243 |
| C104T | 0.00144 |
| C105C | 7.88E−4 |
| C106T | 0.0452 |
| C106C | 0.0294 |
| C107 | 9.89E−4 |
| C108 | 0.00125 |
| B94SR | 0.00264 |
| B94RS | 6.57E−4 |
| B94SS | 0.00363 |
| B94RR | 6.61E−4 |
| C109C | 0.00848 |
| C104C | 0.0113 |
| C111C | 0.00395 |
| C112C | 0.00315 |
| C113T | 0.00111 |
| C113C | 0.00719 |
| C114T | 6.69E−4 |
| C114C | 8.21E−4 |
| C115T | 0.00139 |
| C115C | 9.78E−4 |
| C116 | 0.00195 |
| C117C | 0.00146 |
| C118T | 0.00273 |
| C118C | 0.00221 |
| C119C | 0.00666 |
| C120C | 0.0667 |
| B95R | 0.00164 |
| B95S | 0.00946 |
| C121T | 0.0134 |
| C121C | 0.0238 |
| C122C | 0.0183 |
| C123C | 0.00618 |
| C124C | 0.0159 |
| C125T | 0.00542 |
| C125C | 0.0029 |
| C102T | 0.00577 |
| C126C | 0.029 |

Example 29-1: In Vivo Mouse DSS Studies -Colon Length

In this study, 12-week-old C57BL/6 female mice (Taconic, the Cambridge City) were supplemented with 2.5% DSS (MP Biomedicals, MW 36,000-50,000) in drinking water for days 0-7, and then switched to normal drinking water beginning on day 7 and continued through day 10. Mice were treated once/twice daily by oral gavage with different compounds of Formula (A) (e.g., B15S, B15R, B30R, or B41R) in different dosing levels or with vehicle solution of 2% HPMC, 1% Tween 80 (w/v) in DI water starting on day 0. Mice were sacrificed on day 10 for assessment of colon length. The DSS-induced model of colitis is a widely used animal model for studying the pathogenesis of and potential treatments for UC, which is a primary form of IBD. DSS water intake causes colonic mucosa injury and erosion, gland loss, and infiltration of proinflammatory immune cells in the colon tissue, followed by mucosal repair and tissue remodeling. This is manifested by a shortened colon, the degree of which correlates well with the severity of mucosal injury in this mouse model of DSS-induced experimental colitis.

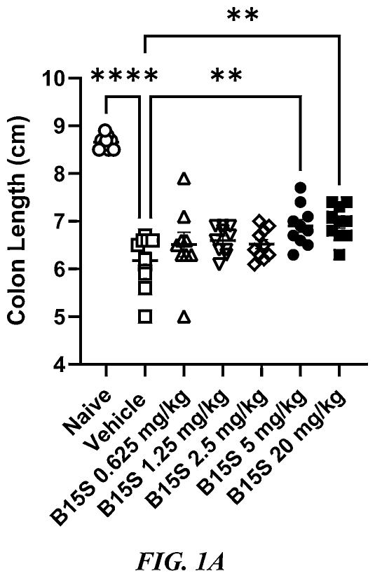

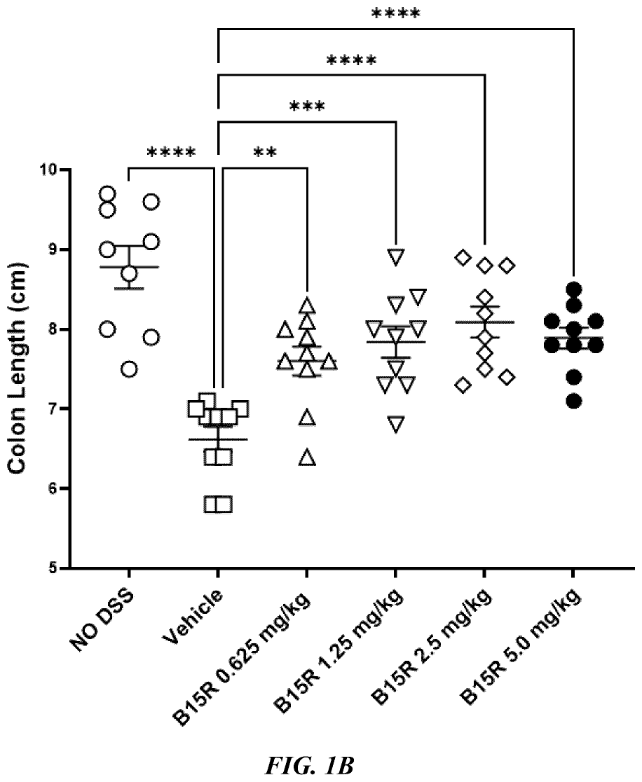

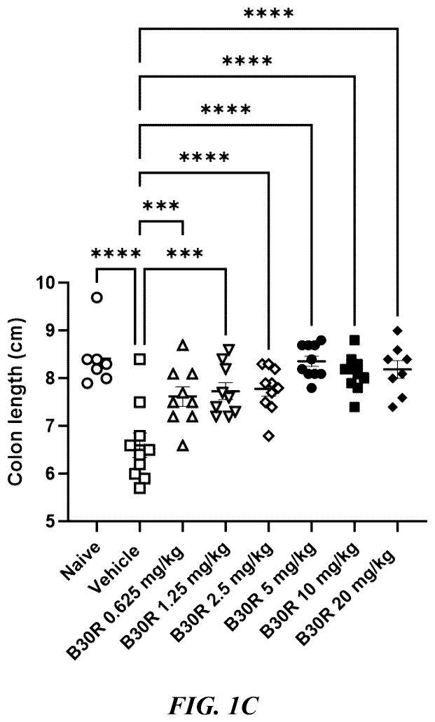

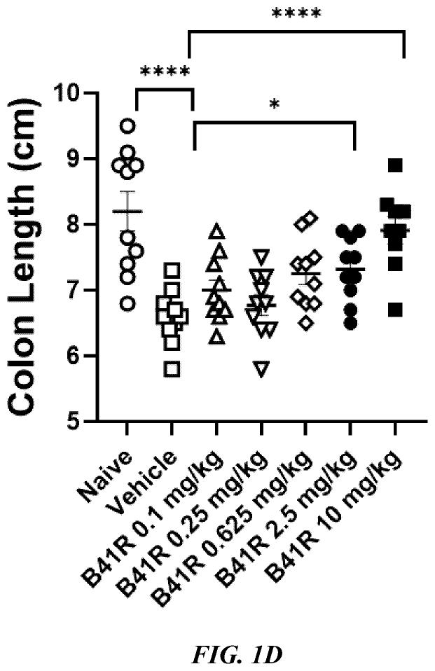

FIG. 1A provides results for mice treated with B 15 S versus vehicle (or naïve mice that were untreated). Treatment with B 15 S at 5 mg/kg and 20 mg/kg once daily showed significant improvement of colon length comparing to vehicle group. Table 29-1 summaries the endpoint mean values of body weight 00 and colon lengths of different groups. The colon length reduction 00 of vehicle and B1SS treatment groups are compared with naïve group. The colon length recovery 00 of B15 S treatment groups are compared with vehicle group.

TABLE 29-1
(B15S)

| | Colon | P value | Reduction | Recovery |
|---|---|---|---|---|
| | length | (vs | % | % (vs |
| Group | Mean (cm) | vehicle) | (vs naïve) | vehicle) |
| Naïve (n = 9) | 8.66 | | | |
| Vehicle (n = 10) | 6.18 | <0.0001 | 28.6 | |
| 0.625 mpk, BID | 6.51 | 0.3926 | 24.8 | 13.4 |
| (n = 10) | | | | |
| 1.25 mpk, QD | 6.60 | 0.1608 | 23.7 | 17.0 |
| (n = 9) | | | | |
| 2.5 mpk, BID (n = 10) | 6.52 | 0.3399 | 24.7 | 13.7 |
| 5 mpk, QD (n = 10) | 6.90 | **0.0031 | 20.3 | 29.0 |
| 20 mpk, QD (n = 10) | 6.97 | **0.0010 | 19.5 | 31.9 |

FIG. 1B provides results for mice treated with B 15R versus vehicle (or naïve mice that were untreated). Treatment with B15R at different doses daily showed significant improvement of colon length comparing to vehicle group. Table 29-2 summaries the endpoint mean values of body weight 00 and colon lengths of different groups. The colon length reduction of vehicle and B15R treatment groups are compared with naïve group. The colon length recovery 00 ofB15R treatment groups are compared with vehicle group. Notably, the B15S S-enantiomer is around 200 times less potent than B 15R in the biochemical assay. The difference in potency shown in DSS efficacy studies is in line with their potency in biochemical and cell assays.

TABLE 29-2
(B15R)

| | Colon | P value | Reduction | Recovery |
|---|---|---|---|---|
| | length | (vs | % | % (vs |
| Group | Mean (cm) | vehicle) | (vs naïve) | vehicle) |
| Naïve (n = 9) | 8.94 | | | |
| Vehicle (n = 10) | 6.62 | <0.0001 | 25.93 | |
| 0.625 mpk, BID | 7.6 | **0.0015 | 14.97 | 42.29 |
| (n = 10) | | | | |
| 1.25 mpk, QD | 7.84 | <0.0001 | 12.28 | 57.64 |
| (n = 10) | | | | |
| 2.5 mpk, BID (n = 10) | 8.09 | <0.0001 | 8.09 | 63.43 |
| 5 mpk, QD (n = 10) | 7.89 | <0.0001 | 11.72 | 54.80 |

As noted above, histopathologic analysis was performed on the mice treated with B 15R. Treatment of mice with B15R resulted in decreased severity of DSS-induced colitis at all doses evaluated for sum of histopathology scores, inflammation scores, erosion scores and gland loss scores, compared to mice exposed to DSS and treated with vehicle.

FIG. 1C provides results for mice treated with B3Rversus vehicle (or naïve mice that were untreated). Treatment with B30R at different doses daily showed significant improvement of colon length comparing to vehicle group. Table 29-3 summaries the endpoint mean values of body weight 00 and colon lengths of different groups. The colon length reduction 00 of vehicle and B30R treatment groups are compared with naïve group. The colon length recovery 00 of B30R treatment groups are compared with vehicle group.

TABLE 29-3
(B30R)

| | Colon | P value | Reduction | Recovery |
|---|---|---|---|---|
| | length | (vs | % | % (vs |
| Group | Mean (cm) | vehicle) | (vs naïve) | vehicle) |
| Naïve (n = 7) | 8.414 | <0.0001 | | |
| Vehicle (n = 10) | 6.60 | | 21.562% | |
| 0.625 mpk, BID | 7.622 | <0.001 | 9.413% | 56.343% |
| (n = 9) | | | | |
| 1.25 mpk, QD | 7.733 | <0.001 | 8.093% | 62.467% |
| (n = 9) | | | | |
| 2.5 mpk, BID (n = 10) | 7.780 | <0.0001 | 7.538% | 65.039% |
| 5 mpk, QD (n = 10) | 8.360 | <0.0001 | 0.645% | 97.008% |
| 10 mpk, QD (n = 10) | 8.110 | <0.0001 | 3.616% | 83.228% |
| 20 mpk, QD (n = 8) | 8.188 | <0.0001 | 2.265% | 87.5% |

FIG. 1D provides results for mice treated with 1B41R versus vehicle (or naïve mice that were untreated). Treatment with B41R at 2.5 mg/kg and 10 mg/kg daily showed significant improvement of colon length comparing to vehicle group. Table 29-4 summaries the endpoint mean values of body weight 00 and colon lengths of different groups. The colon length reduction 00 of vehicle and B41R treatment groups are compared with naïve group. The colon length recovery 00 of B41R treatment groups are compared with vehicle group.

TABLE 29-4
(B41R)

| | Colon | P value | Reduction | Recovery |
|---|---|---|---|---|
| | length | (vs | % | % (vs |
| Group | Mean (cm) | vehicle) | (vs naïve) | vehicle) |
| Naïve (n = 10) | 8.2 | <0.0001 | | |
| Vehicle (n = 10) | 6.59 | | 19.63% | |
| 0.1 mpk, QD (n = 10) | 7.0 | 0.4570 | 14.63% | 25.46% |
| 0.25 mpk, QD (n = 10) | 6.8 | 0.9735 | 17.44% | 11.18% |
| 0.625 mpk, QD (n = 10) | 7.3 | 0.0697 | 11.59% | 40.99% |
| 2.5 mpk, QD (n = 10) | 7.3 | 0.0355 | 10.73% | 45.34% |
| 10 mpk, QD (n = 10) | 7.9 | <0.0001 | 3.52% | 82.06% |

Example 29-2: In Vivo Mouse DSS Studies -Histopathological Analysis

Histopathological analysis was performed on the C57B3L/6 mice from the DSS study described above. Analysis was performed on mice from the B15R, B30R, or B41R groups. Briefly, mouse colon tissues were collected at necropsy, flushed with PBS, manipulated intact into individual modified Swiss rolls, placed into separate tissue cassettes and fixed for approximately 24 hours by immersion in 10% neutral buffered formalin followed by transfer to 7000 ethanol. Fixed tissues were routinely processed, paraffin-embedded, sectioned, mounted on glass slides and hematoxylin and eosin stained. Glass slides were scanned and digital images were accessed for histopathology evaluation. Prior to histopathology assessment, metadata for all digital images reviewed was exported from a digital pathology platform database into an Excel spreadsheet, followed by assignment of each digital image with a random number (greater than 0 and less than 1) using the RAND function in Excel. From smallest to largest random number assigned, each digital image was retrieved for evaluation using only the RTLIMS Inventory Code as the unique identifier for each digital image. Each digital image was individually reviewed without knowledge of treatment group (blinded/masked evaluation), and with the slide label image hidden when viewing digital images. Upon completion of histopathology evaluation and scoring, the slide label image was revealed, and animal ID recorded and verified to be associated with the RTLIMS Inventory Code unique identifier. Scoring of tissues for DSS-induced colitis was performed using a scoring system and criteria modified and adapted from BolderBioPATH, Inc./Inotiv. Ordinal data (histopathology scores) were analyzed using a non-parametric Kruskal-Wallis one-way analysis of variance test followed by a Dunn's multiple comparison test (GraphPad Prism).

The following scoring systems were used to provide histological scores:

TABLE 29-5
Inflammation Scoring

| Score | Inflammation Scoring Criteria |
|---|---|
| 0 | No inflammation. |
| 0.5 | Very minimal: 1 or 2 small foci of mononuclear inflammatory |
| | cells, affecting less than 1% of the mucosa. |
| 1 | Minimal: larger focal areas of mononuclear inflammatory cells |
| | and neutrophils affecting 1-10% of the mucosa or minimal |
| | diffuse, may be mostly in areas of submucosal edema. |
| 2 | Mild: diffuse mild or multifocal, affecting 11-25% of the |
| | mucosa with minor focal or multifocal gland separation; |
| | no separation in most areas. |
| 3 | Moderate: 26-50% of mucosa affected with minimal to mild |
| | focal or multifocal separation of glands by inflammatory |
| | cell infiltrate, milder in remaining areas of mucosa with |
| | some areas having no gland separation by inflammation. |
| 4 | Marked: 51-75% of mucosa affected with mild to moderate |
| | separation of glands by inflammatory cell infiltrate, |
| | minimal to mild in remaining areas of mucosa but all glands |
| | have some separation by infiltrate. |
| 5 | Severe: 76-100% of mucosa affected with moderate to marked |
| | areas of gland separation by inflammatory cell infiltrate; |
| | mild to moderate in remaining areas of mucosa. |

TABLE 29-6
Gland Loss Scoring

| Score | Gland Loss Scoring Criteria |
|---|---|
| 0 | No gland loss. |
| 0.5 | Very minimal: 1 or 2 small focal areas of gland |
| | loss affecting less than 1% of the mucosa. |
| 1 | Minimal: 1-10% of the mucosa affected. |
| 2 | Mild: 11-25% of the mucosa affected. |
| 3 | Moderate: 26-50% of the mucosa affected. |
| 4 | Marked: 51-75% of the mucosa affected. |
| 5 | Severe: 76-100% of the mucosa affected. |

TABLE 29-7
Erosion Scoring

| Score | Erosion Scoring Criteria |
|---|---|
| 0 | No erosion. |
| 0.5 | Very minimal: 1 or 2 small focal areas of erosion |
| | affecting less than 1% of the mucosa. |
| 1 | Minimal: 1-10% of the mucosa affected. |
| 2 | Mild: 11-25% of the mucosa affected. |
| 3 | Moderate: 26-50% of the mucosa affected. |
| 4 | Marked: 51-75% of the mucosa affected. |
| 5 | Severe: 76-100% of the mucosa affected. |

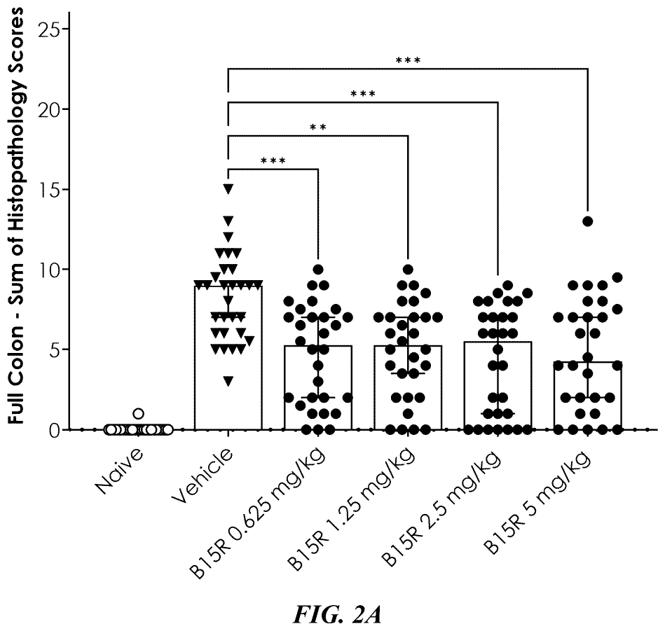

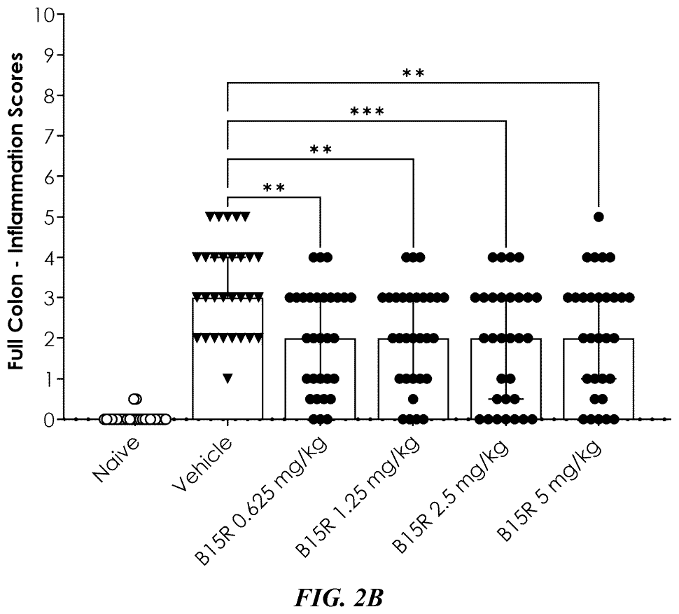

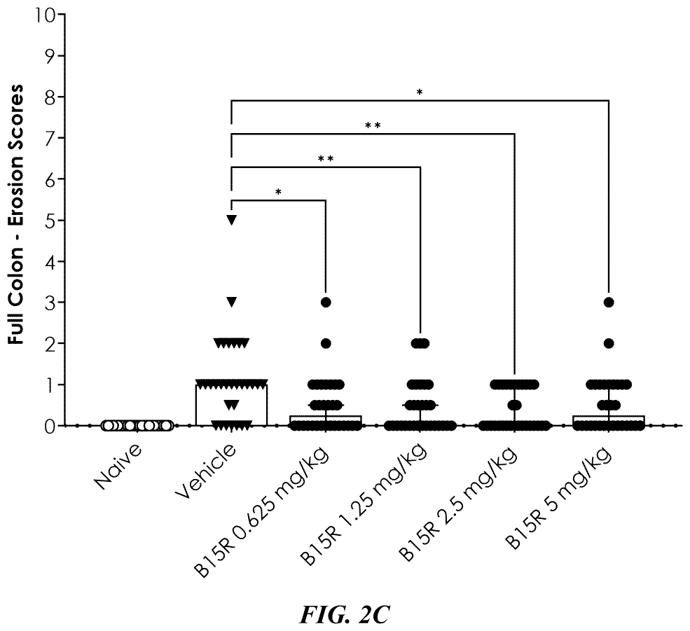

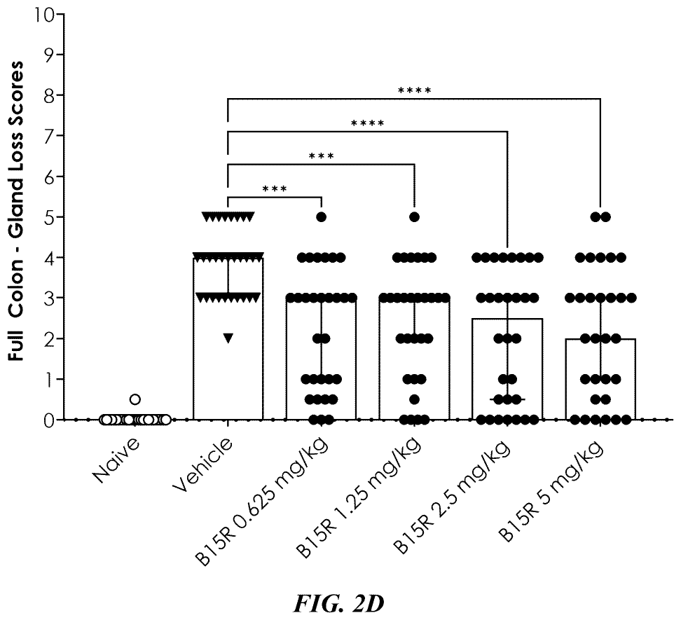

Compared to mice exposed to DSS and treated with vehicle, treatment of mice with B15R resulted in decreased severity of DSS-induced colitis at all doses evaluated for sum of histopathology scores (FIG. 2A), inflammation scores (FIG. 2B), erosion scores (FIG. 2C) and gland loss scores (FIG. 2D).

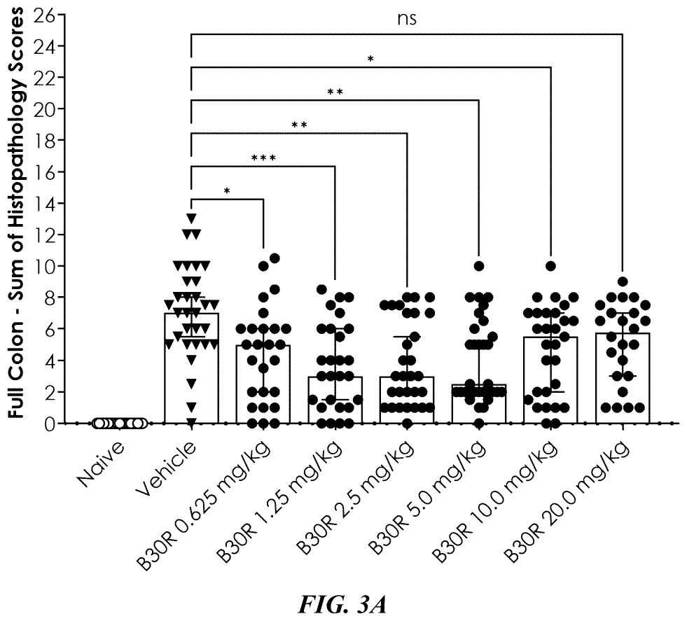

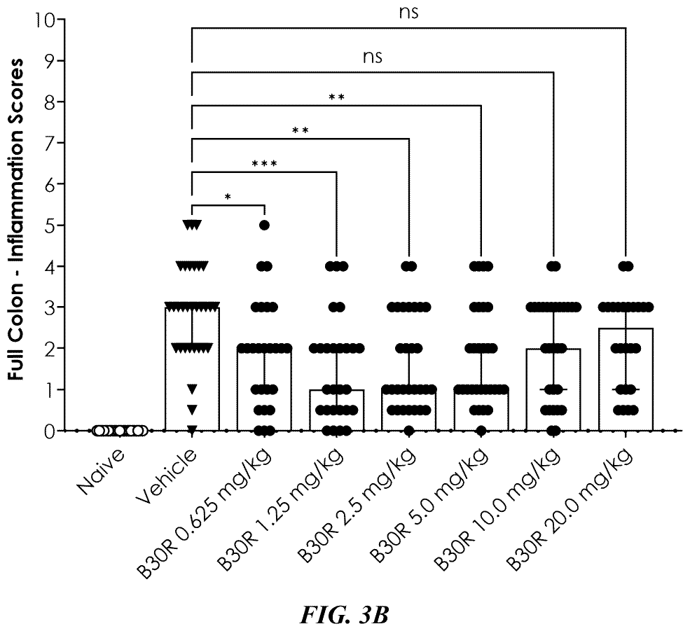

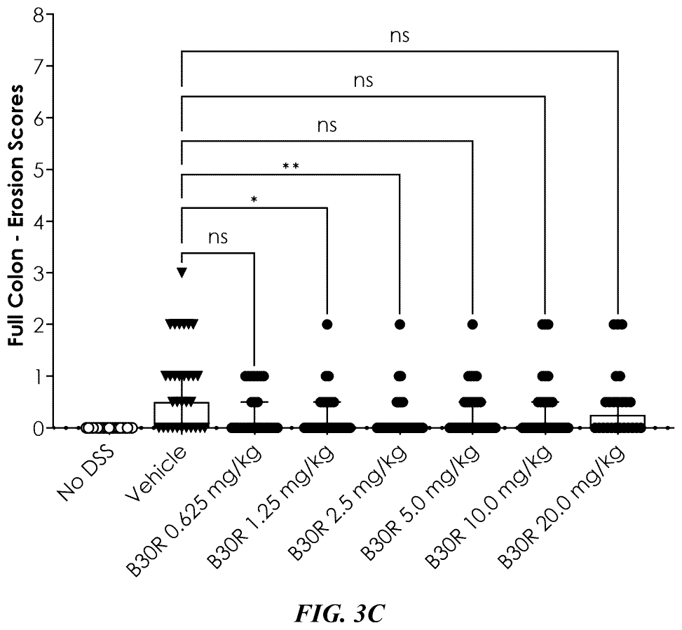

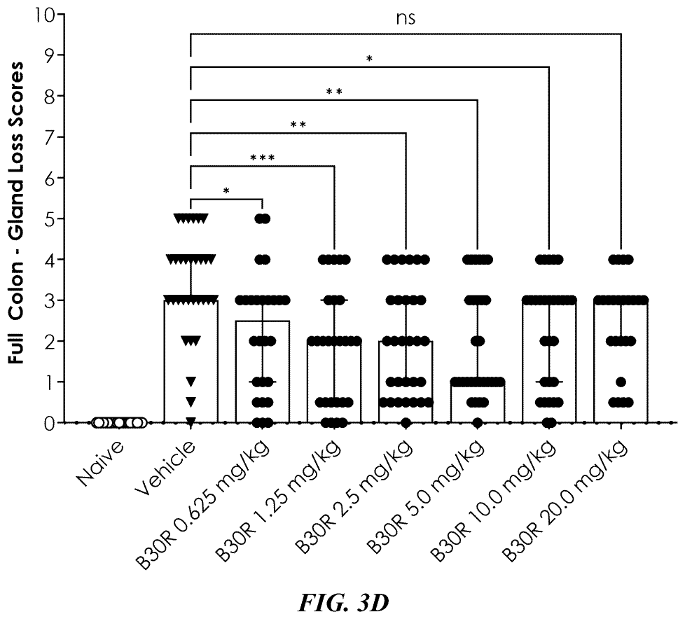

Compared to mice exposed to DSS and treated with vehicle, treatment of mice with B30R resulted in decreased severity of full colon DSS-induced colitis at all doses evaluated for sum of histopathology scores (FIG. 3A), inflammation scores (FIG. 3B) and erosion scores (FIG. 3C) and at 0.625 mg/kg BID, 1.25 mg/kg QD, 2.5 mg/kg BID and 5 mg/kg QD for gland loss scores (FIG. 3D).

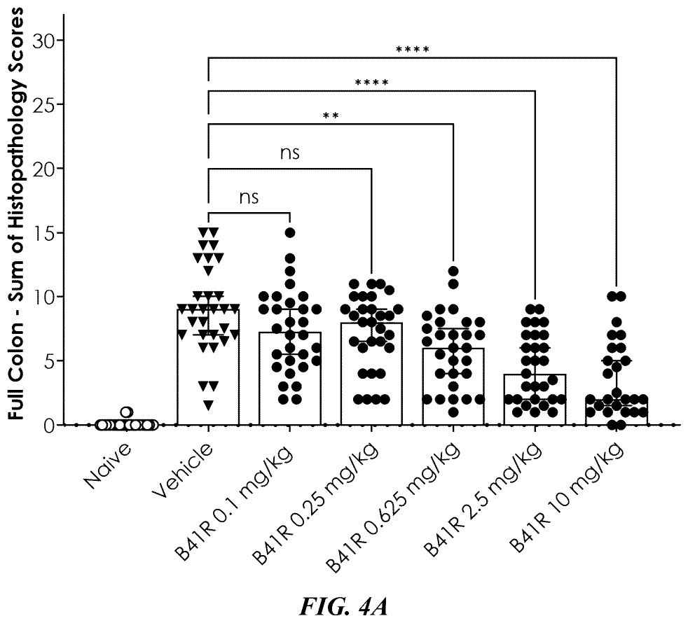

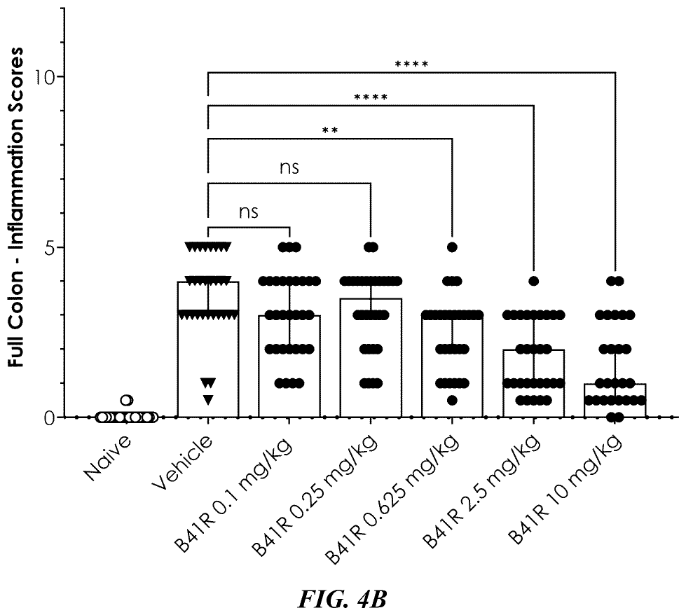

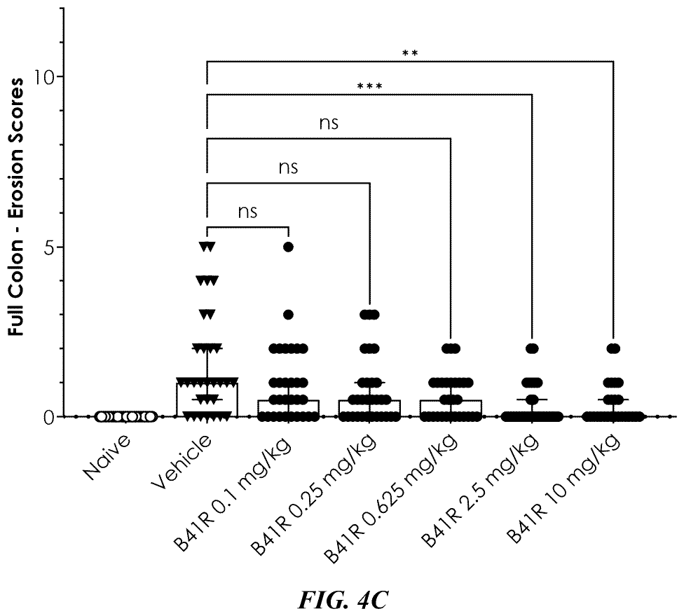

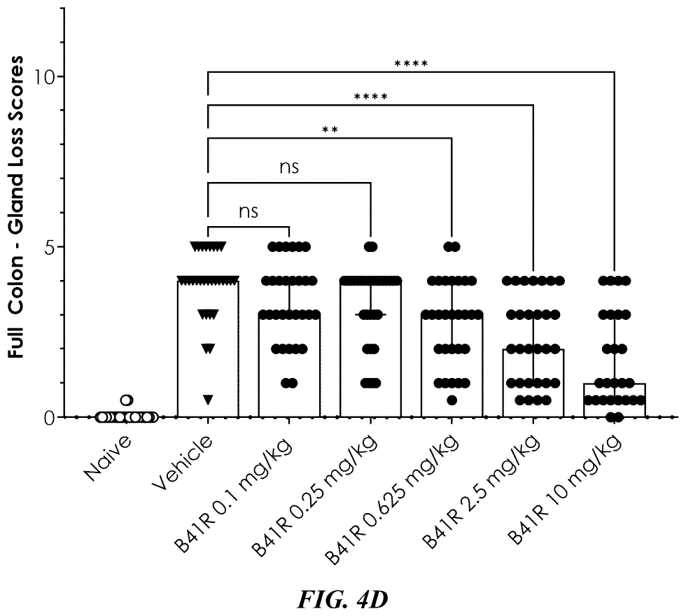

Compared to mice exposed to DSS and treated with vehicle, treatment of mice with B41R resulted in decreased severity of full colon DSS-induced colitis at all doses evaluated for sum of histopathology scores (FIG. 4A), inflammation scores (FIG. 4B) and erosion scores (FIG. 4C) and at 0.1 mg/kg QD and >0.625 mg/kg QD for gland loss scores (FIG. 4D).

1. A compound of Formula (A) 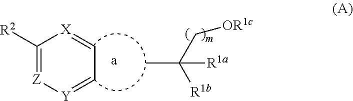
or a pharmaceutically acceptable salt of said compound; 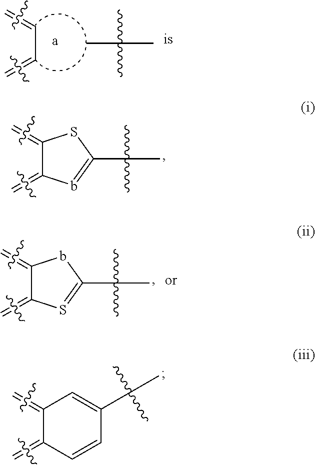
wherein
m is 0 or 1;
X is N or CH;
Y is N or C—R³;
Z is N or C—R⁵;
b is N or C—R⁴;
R^{1a} is —H, deuterium, C₁₋₆ alkyl, or C₁₋₆ haloalkyl;
wherein, when R^{1a} is C₁₋₆ alkyl or C₁₋₆ haloalkyl, then R^{1a} may be unsubstituted or substituted with one or more substituents, each of which independently is halogen, —OH, —N(R^{b})₂, C₁₋₃ alkyl, or C₁₋₃ alkoxy;
R^{1b} is —H, C₁₋₆ alkyl, C₁₋₆ haloalkyl, C₁₋₆ heteroalkyl, —(C₁₋₆ alkylene)-O-(C₁₋₆ alkyl), C₃₋₁₀ cycloalkyl, or heterocyclyl having 3 to 10 ring members;
wherein, when R^{1b} is C₁₋₆ alkyl, C₁₋₆ haloalkyl, C₁₋₆ heteroalkyl, —(C₁₋₆ alkylene)-O-(C₁₋₆ alkyl), C₃₋₁₀ cycloalkyl, or heterocyclyl having 3 to 10 ring members, then R^{1b} may be unsubstituted or substituted with one or more substituents, each of which independently is halogen, —OH, —N(R^{b})₂, C₁₋₃ alkyl, or C₁₋₃ alkoxy; wherein each of the C₁₋₃ alkyl and C₁₋₃ alkoxy may be unsubstituted or substituted with 1 to 6—F groups;
or, alternatively, R^{1a} and R^{1b} together form a C₃₋₁₀ cycloalkyl, C₃₋₁₀ cycloalkenyl, or heterocyclyl having 3 to 10 ring members;
wherein the R^{1a} and R^{1b} cycle may be unsubstituted or substituted with one or more substituents, each of which independently is halogen, —OH, oxo, —N(R^{b})₂, C₁₋₃ alkyl, C₁₋₃alkoxy, -CN, C₁₋₆heteroalkyl, C₃₋₁₀ cycloalkyl, or heterocyclyl having 3 to 10 ring members; wherein each of the C₁₋₃ alkyl and C₁₋₃ alkoxy may be unsubstituted or substituted with 1 to 9 halogen groups or C₁₋₃ alkoxy;
R^{1c} is —H, C₁₋₆ alkyl, or C₃₋₆ cycloalkyl;
wherein, when R^{1c} is C₁₋₆ alkyl or C₃₋₆ cycloalkyl, then R^{1c} may be unsubstituted or substituted with one or more substituents, each of which independently is halogen, —OH, —N(R^{b})₂, C₁₋₃ alkyl, C₁₋₃ alkoxy, or C₁₋₆ heteroalkyl;
R² is C₃₋₂₀ cycloalkyl, C₃₋₂₀ cycloalkenyl, C₆₋₁₈ aryl, heteroaryl having 5 to 20 ring members, or heterocyclyl having 3 to 20 ring members;
wherein R² may be unsubstituted or substituted with one or more substituents, each of which independently is halogen, —OH, oxo, —N(R^{b})₂, C₁₋₆ alkyl, C₁₋₆ haloalkyl, C₁₋₆ alkoxy, C₁₋₆alkylene-OH, C₁₋₆alkylene-O-C₁₋₆alkyl, or C₁₋₆heteroalkyl;
R³ is —H, C₁₋₆ alkyl, C₁₋₆ haloalkyl, C₃₋₆ cycloalkyl, amino, C₁₋₄alkyl-amino, di-C₁₋₄alkyl-amino, C₆₋₁₀aryl, heteroaryl having 5 to 10 ring members, or heterocyclyl having 5 to 10 ring members;
wherein, when R³ is C₁₋₆ alkyl, C₁₋₆ haloalkyl, C₃₋₆ cycloalkyl, C₁₋₄alkyl-amino, di-C₁₋₄ alkyl-amino, C₆₋₁₀aryl, heteroaryl having 5 to 10 ring members, or heterocyclyl having 5 to 10 ring members, then R³ may be unsubstituted or substituted with one or more substituents, each of which independently is halogen, —OH, oxo, —N(R^{b})₂, C₁₋₃ alkyl, C₁₋₃ haloalkyl, C₁₋₃ alkoxy, C₁₋₃ alkylene-C₁₋₃ alkoxy, C₁₋₆ heteroalkyl, or two R³ substituents together form a C₃₋₆ carbocyclyl or a heterocyclyl having 3 to 6 ring members;
R⁴ is —H, C₁₋₃ alkyl, C₁₋₃ haloalkyl, —CN, halogen, or —N(R^{a})₂ where each instance of R^{a}, where present, independently is —H, C₁₋₆ alkyl, or —C(O)C₁₋₁₆ alkyl; or, alternatively, two R^{a} substituents together form a heterocyclyl having 3 to 6 ring members;
wherein R^{a} may be unsubstituted or substituted with one or more substituents, each of which independently is —F, —Cl, —OH, C₁₋₃ alkoxy;
R⁵ is —H, C₁₋₆ alkyl, C₃₋₆ cycloalkyl, C₁₋₆ haloalkyl, or C₁₋₆ heteroalkyl;
wherein, when R⁵ is C₁₋₁₆ alkyl, C₃₋₆ cycloalkyl, C₁₋₁₆ haloalkyl, or C₁₋₆heteroalkyl, then R⁵ may be unsubstituted or substituted one or more substituents, each of which independently is halogen, —OH, C₁₋₃ alkyl, or C₁₋₃ alkoxy;
where each instance of R^{b}, where present, independently is —H or C₁₋₆ alkyl; or, alternatively, two R^{b} substituents together form a heterocyclyl having 3 to 6 ring members; and
wherein, when R^{b} is C₁₋₆ alkyl or, alternatively, two R^{b} substituents together form a heterocyclyl having 3 to 6 ring members, then R^{b} may be unsubstituted or substituted with one or more substituents, each of which independently is —F, —Cl, —OH, C₁₋₃ alkoxy;
provided that:
a) if 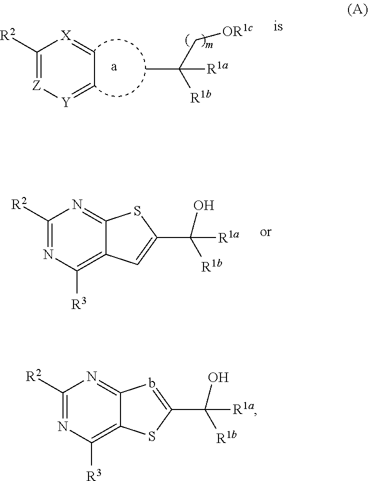
then R³ is not unsubstituted or substituted morpholinyl; and
b) if 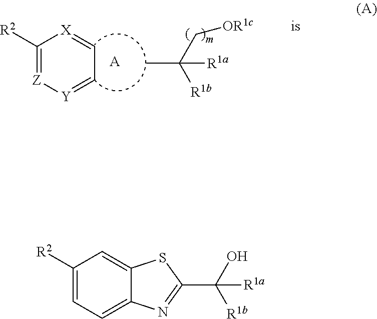
then R^{1a} and R^{1b} are not both methyl, R^{1a} and R^{1b} are not both —CF₃, R^{1a} is not —CF₃ when R^{1b} is methyl, R^{1b} is not —CF₃ when R^{1a} is methyl, and R^{1a} and R^{1b} together do not provide unsubstituted or substituted piperdinyl.

2-3. (canceled)

4. The compound or salt of claim 1, wherein

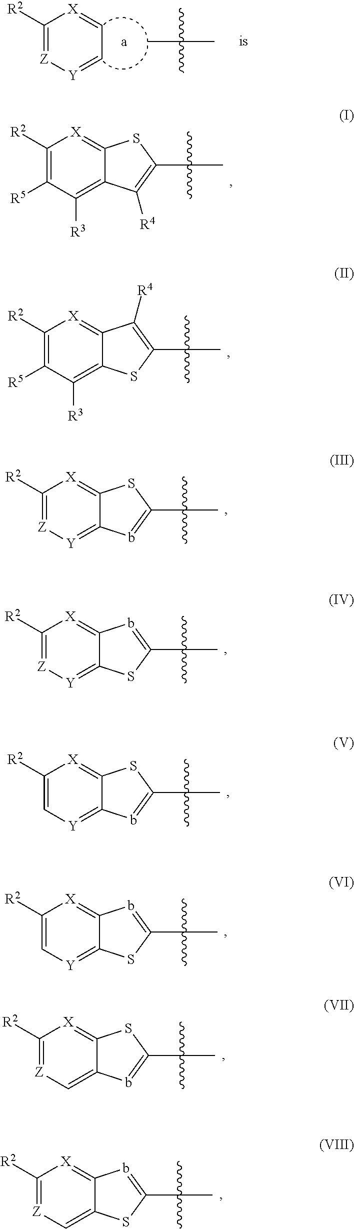

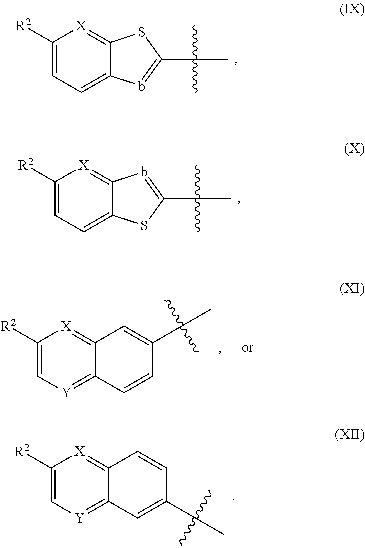

5. The compound or salt of claim 4, wherein

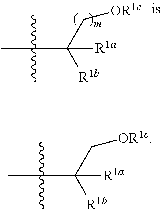

6. The compound of claim 1, wherein the compound of Formula (A) is a compound of Formula (I) 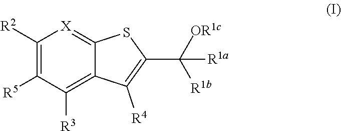
or a pharmaceutically acceptable salt of said compound;
where
X is N or CH;
R^{1a} is —H, C₁₋₆ alkyl, or C₁₋₆ haloalkyl;
wherein, when R^{1a} is C₁₋₆alkyl or C₁₋₆haloalkyl, then R^{1a} may be unsubstituted or substituted with one or more substituents, each of which independently is halogen, —OH, —N(R^{b})₂, C₁₋₃ alkyl, or C₁₋₃ alkoxy;
R^{1b} is —H, C₁₋₆ alkyl, C₁₋₆ haloalkyl, C₁₋₆ heteroalkyl, —(C₁₋₆ alkylene)-O-(C₁₋₆ alkyl), C₃₋₁₀ cycloalkyl, or heterocyclyl having 3 to 10 ring members;
wherein, when R^{1b} is C₁₋₆ alkyl, C₁₋₆ haloalkyl, C₁₋₆ heteroalkyl, C₃₋₁₀ cycloalkyl, or heterocyclyl having 3 to 10 ring members, then R^{1b} may be unsubstituted or substituted with one or more substituents, each of which independently is halogen, —OH, —N(R^{b})₂, C₁₋₃ alkyl, or C₁₋₃ alkoxy; wherein each of the C₁₋₃ alkyl and C₁₋₃ alkoxy may be unsubstituted or substituted with 1 to 6—F groups;
or, alternatively, R^{1a} and R^{1b} together form a C₃₋₀ cycloalkyl, C₃₋₁₀ cycloalkenyl, or heterocyclyl having 3 to 10 ring members;
wherein the R^{1a} and R^{1b} cycle may be unsubstituted or substituted with one or more substituents, each of which independently is halogen, —OH, oxo, —N(R^{b})₂, C₁₋₃ alkyl, C₁₋₃alkoxy, or C₁₋₆ heteroalkyl; wherein each of the C₁₋₃ alkyl and C₁₋₃ alkoxy may be unsubstituted or substituted with 1 to 9 halogen groups or C₁₋₃ alkoxy;
R^{1c} is —H, C₁₋₆ alkyl, or C₃₋₆ cycloalkyl;
wherein, when R^{1c} is C₁₋₆ alkyl or C₃₋₆ cycloalkyl, R^{1c} may be unsubstituted or substituted with one or more substituents, each of which independently is halogen, —OH, —N(R^{b})₂, C₁₋₃ alkyl, C₁₋₃ alkoxy, or C₁₋₆heteroalkyl;
R² is C₃₋₂₀ cycloalkyl, C₃₋₂₀ cycloalkenyl, C₆₋₁₈ aryl, heteroaryl having 5 to 20 ring members, or heterocyclyl having 3 to 20 ring members;
wherein R² may be unsubstituted or substituted with one or more substituents, each of which independently is halogen, —OH, oxo, —N(R^{b})₂, C₁₋₆ alkyl, C₁₋₆ haloalkyl, C₁₋₆ alkoxy, C₁₋₆alkylene-OH, C₁₋₆alkylene-O-C₁₋₆alkyl, or C₁₋₆heteroalkyl;
R³ is —H, C₁₋₆ alkyl, C₁₋₆ haloalkyl, C₃₋₆ cycloalkyl, amino, C₁₋₄alkyl-amino, di-C₁₋₄alkyl-amino, C₆₋₁₀aryl, heteroaryl having 5 to 10 ring members, or heterocyclyl having 5 to 10 ring members;
wherein, when R³ is C₁₋₆ alkyl, C₁₋₆ haloalkyl, C₃₋₆ cycloalkyl, C₁₋₄alkyl-amino, di-C₁₋₄ alkyl-amino, C₆₋₁₀aryl, heteroaryl having 5 to 10 ring members, or heterocyclyl having 5 to 10 ring members, then R³ may be unsubstituted or substituted with one or more substituents, each of which independently is halogen, —OH, oxo, —N(R^{b})₂, C₁₋₃ alkyl, C₁₋₃ haloalkyl, C₁₋₃ alkoxy, C₁₋₃ alkylene-C₁₋₃ alkoxy, C₁₋₆ heteroalkyl, or two R³ substituents together form a C₃₋₆ carbocyclyl or a heterocyclyl having 3 to 6 ring members;
R⁴ is —H or —N(R^{a})₂ where each instance of R^{a}, where present, independently is —H or C₁₋₆ alkyl; or, alternatively, two R^{a} substituents together form a heterocyclyl having 3 to 6 ring members;
wherein R^{a} may be unsubstituted or substituted with one or more substituents, each of which independently is —F, —Cl, —OH, C₁₋₃ alkoxy;
R⁵ is —H, C₁₋₆ alkyl, C₃₋₆ cycloalkyl, C₁₋₆ haloalkyl, or C₁₋₆ heteroalkyl;
wherein, when R⁵ is C₁₋₆ alkyl, C₃₋₆ cycloalkyl, C₁₋₆ haloalkyl, or C₁₋₆heteroalkyl, then R⁵ may be unsubstituted or substituted one or more substituents, each of which independently is halogen, —OH, C₁₋₃ alkyl, or C₁₋₃ alkoxy;
where each instance of R^{b}, where present, independently is —H or C₁₋₆ alkyl; or, alternatively, two R^{b} substituents together form a heterocyclyl having 3 to 6 ring members; and
wherein, when R^{b} is C₁₋₆ alkyl or, alternatively, two R^{b} substituents together form a heterocyclyl having 3 to 6 ring members, then R^{b} may be unsubstituted or substituted with one or more substituents, each of which independently is —F, —Cl, —OH, C₁₋₃ alkoxy;
provided that the compound is not:
6-phenyl-4-(trifluoromethyl)thienol[2,3-b]pyridine-2-methanol;
6-cyclopropylbenzol[b]thiophene-2-methanol;
6-phenylbenzo[b]thiophene-2-methanol; or
6-[3-[(dimethylamino)methyl]bicyclo[2.2.1]hept-2-en-2-yl]benzo[b]thiophene-2-methanol.

7-8. (canceled)

9. The compound or salt of claim 4, wherein R^{1a} is —H, unsubstituted or substituted C₁₋₃alkyl, or unsubstituted or substituted C₁₋₃haloalkyl.

10. (canceled)

11. The compound or salt of claim 4, wherein, when R^{1a} is substituted, each R^{1a} substituent independently is —F, —OH, or C₁₋₃alkoxy.

12-13. (canceled)

14. The compound or salt of claim 4, wherein R^{1b} is —H, unsubstituted or substituted C₁₋₃ alkyl, unsubstituted or substituted C₁₋₃ haloalkyl, unsubstituted or substituted C₃₋₆ cycloalkyl, or unsubstituted or substituted heterocyclyl having 3- to 6-ring members.

15-23. (canceled)

24. The compound or salt of claim 4, wherein R^{1a} and R^{1b} together form an unsubstituted or substituted C₃₋₁₀ cycloalkyl or an unsubstituted or substituted 3 to 10 membered heterocyclyl.

25. (canceled)

26. The compound or salt of claim 24, wherein, when R^{1a} and R^{1b} together form an unsubstituted or substituted heterocyclyl, the heterocyclyl includes 1, 2, or 3 heteroatom ring members, each of which independently is nitrogen, oxygen, or —S(O)ₓ—, where x is 0, 1, or 2.

27. The compound or salt of claim 4, wherein R^{1a} and R^{1b} together form an unsubstituted or substituted C₃₋₁₀ cycloalkyl.

28. The compound or salt of claim 24, wherein R^{1a} and R^{1b} together form a spiro ring system.

29. The compound or salt of claim 24, wherein R^{1a} and R^{1b} together form a bicyclic ring system.

30-31. (canceled)

32. The compound or salt of claim 24, wherein R^{1a} and R^{1b} together form a cycle that is substituted and each substituent of the cycle independently is halogen, —OH, oxo, —N(R^{b})₂, C₁₋₃ alkyl, or C₁₋₃alkoxy, wherein each of the C₁₋₃ alkyl and C₁₋₃ alkoxy may be unsubstituted or substituted with 1 to 6 halogen groups or C₁₋₃ alkoxy.

33. The compound or salt of claim 24, wherein R^{1a} and R^{1b} together form a cycle that is substituted and each substituent of the cycle independently is —F, —OH, oxo, methyl, methoxy, or -OCF₃.

34. (canceled)

35. The compound or salt of claim 4, wherein R^{1c} is —H.

36-38. (canceled)

39. The compound or salt of claim 4, wherein R² is unsubstituted or substituted heteroaryl having 1, 2, 3, or 4 heteroatom ring members.

40. (canceled)

41. The compound or salt of claim 39, wherein R² is unsubstituted or substituted fused bicyclic heteroaryl.

42. The compound or salt of claim 41, wherein R² is unsubstituted or substituted heteroaryl having 8 to 10 ring-members.

43-44. (canceled)

45. The compound or salt of claim 42, wherein, when R² is substituted, each R² substituent independently is halogen, —OH, oxo, C₁₋₃alkyl, or C₁₋₃ alkoxy.

46-48. (canceled)

49. The compound or salt of any one of claim 4, wherein R² is

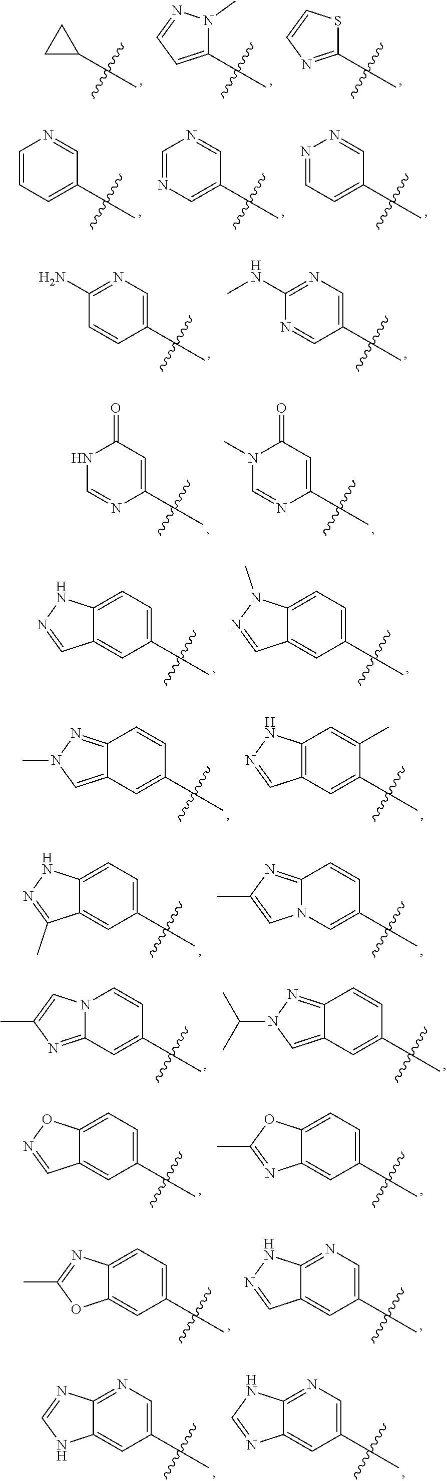

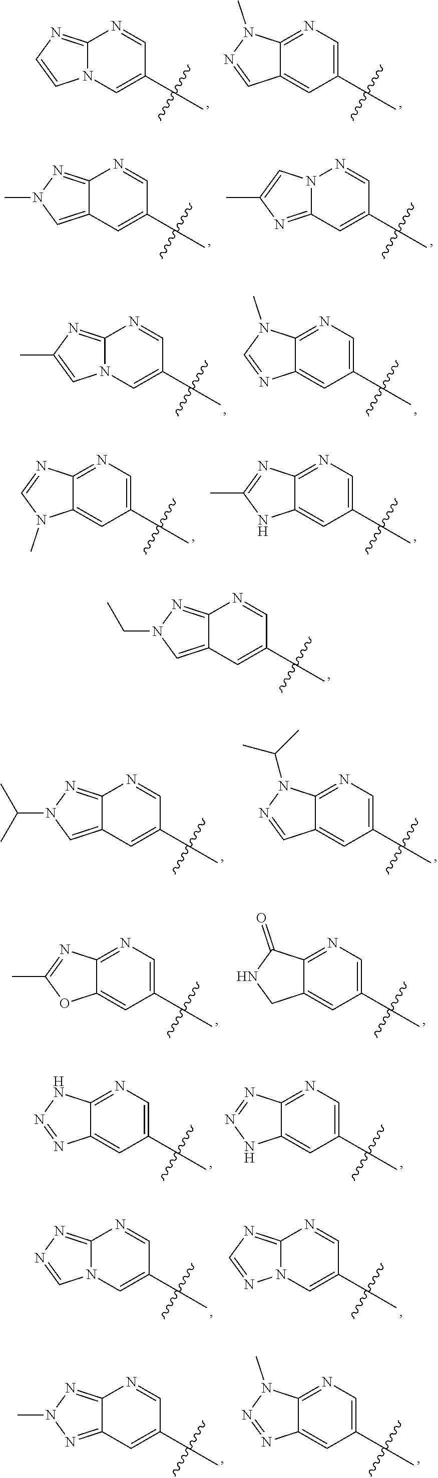

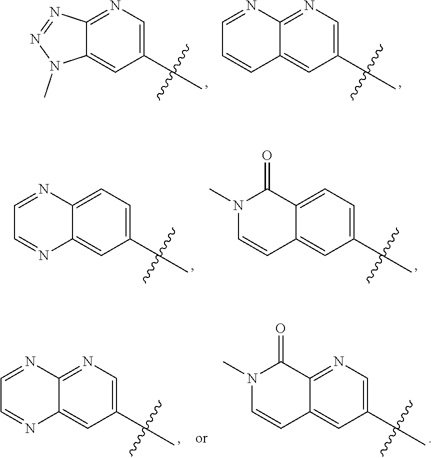 [SMILES: *c1cnc2nnn(C)c2c1 ; *c1cnc2ncccc2c1 ; *c1ccc2nccnc2c1 ; *c1ccc2c(=O)n(C)ccc2c1 ; *c1cnc2nccnc2c1 ; *c1cnc2c(=O)n(C)ccc2c1]

50-59. (canceled)

60. The compound or salt of claim 1, wherein R³ is —H.

61. The compound or salt of claim 1, wherein R⁴ is —H, C₁₋₃ alkyl, halogen, or —N(R^{a})₂ where each instance of R^{a}, where present, independently is —H or C₁₋₆ alkyl.

62-65. (canceled)

66. The compound or salt of claim 1, wherein R⁵ is —H.

67. The compound or salt of claim 1, wherein the compound is:
5-(4-cyclobutyl-2-(methoxymethyl)thieno [2,3-b]pyridin-6-yl)-2-methyl-2H-pyrazolo [3,4-b]pyridine;
5-(2-(methoxymethyl)-4-(1-(methoxymethyl)cyclobutyl)thieno[2,3-b]pyridin-6-yl)-2-methyl-2H-pyrazolo [3,4-b]pyridine;
(R)-(4,6-bis(1-methyl-1H-pyrazol-5-yl)thieno[2,3-b]pyridin-2-yl)(cyclobutyl)methanol;
(S)-(4,6-bis(1-methyl-1H-pyrazol-5-yl)thieno[2,3-b]pyridin-2-yl)(cyclobutyl)methanol;
(R)-cyclobutyl(6-(2-methyl-2H-pyrazolo[3,4-b]pyridin-5-yl)-4-(1-methyl-1H-pyrazol-5-yl)thieno [2,3-b]pyridin-2-yl)methanol;
(S)-cyclobutyl(6-(2-methyl-2H-pyrazolo[3,4-b]pyridin-5-yl)-4-(1-methyl-1H-pyrazol-5-yl)thieno [2,3-b]pyridin-2-yl)methanol;
(R)-cyclobutyl(4-(2-methyl-2H-pyrazolo[3,4-b]pyridin-5-yl)-6-(1-methyl-1H-pyrazol-5-yl)thieno [2,3-b]pyridin-2-yl)methanol;
(S)-cyclobutyl(4-(2-methyl-2H-pyrazolo[3,4-b]pyridin-5-yl)-6-(1-methyl-1H-pyrazol-5-yl)thieno [2,3-b]pyridin-2-yl)methanol;
5-(2-((R)-cyclobutyl(methoxy)methyl)-4-(1-methyl-1H-pyrazol-5-yl)thieno[2,3-b]pyridin-6-yl)-2-methyl-2H-pyrazolo [3,4-b]pyridine;
5-(2-((S)-cyclobutyl(methoxy)methyl)-4-(1-methyl-1H-pyrazol-5-yl)thieno[2,3-b]pyridin-6-yl)-2-methyl-2H-pyrazolo[3,4-b]pyridine;
(1R)-1-cyclobutyl-1-(6-(2-methyl-2H-pyrazolo[3,4-b]pyridin-5-yl)-4-(1-methyl-1H-pyrazol-5-yl)thieno [2,3-b]pyridin-2-yl)ethanol;
(1S)-1-cyclobutyl-1-(6-(2-methyl-2H-pyrazolo[3,4-b]pyridin-5-yl)-4-(1-methyl-1H-pyrazol-5-yl)thieno [2,3-b]pyridin-2-yl)ethanol;
(1R)-1-cyclobutyl-2,2,2-trifluoro-1-(6-(2-methyl-2H-pyrazolo[3,4-b]pyridin-5-yl)-4-(1-methyl-1H-pyrazol-5-yl)thieno [2,3-b]pyridin-2-yl)ethanol;
(1S)-1-cyclobutyl-2,2,2-trifluoro-1-(6-(2-methyl-2H-pyrazolo[3,4-b]pyridin-5-yl)-4-(1-methyl-1H-pyrazol-5-yl)thieno [2,3-b]pyridin-2-yl)ethanol;
(1R)-1-(4-(2-methyl-2H-pyrazolo[3,4-b]pyridin-5-yl)-6-(1-methyl-1H-pyrazol-5-yl)thieno[2,3-b]pyridin-2-yl)ethanol;
(1S)-1-(4-(2-methyl-2H-pyrazolo[3,4-b]pyridin-5-yl)-6-(1-methyl-1H-pyrazol-5-yl)thieno[2,3-b]pyridin-2-yl)ethanol;
(1R)-1-(6-(2-methyl-2H-pyrazolo[3,4-b]pyridin-5-yl)-4-(1-methyl-1H-pyrazol-5-yl)thieno[2,3-b]pyridin-2-yl)ethanol;
(1S)-1-(6-(2-methyl-2H-pyrazolo[3,4-b]pyridin-5-yl)-4-(1-methyl-1H-pyrazol-5-yl)thieno[2,3-b]pyridin-2-yl)ethanol;
(R)-cyclobutyl(6-cyclopropyl-4-(2-methyl-2H-pyrazolo [3,4-b]pyridin-5-yl)thieno [2,3-b]pyridin-2-yl)methanol;
(S)-cyclobutyl(6-cyclopropyl-4-(2-methyl-2H-pyrazolo [3,4-b]pyridin-5-yl)thieno [2,3-b]pyridin-2-yl)methanol;
(R)-cyclobutyl(4-cyclopropyl-6-(2-methyl-2H-pyrazolo [3,4-b]pyridin-5-yl)thieno [2,3-b]pyridin-2-yl)methanol;
(S)-cyclobutyl(4-cyclopropyl-6-(2-methyl-2H-pyrazolo [3,4-b]pyridin-5-yl)thieno [2,3-b]pyridin-2-yl)methanol;
(4-(dimethylamino)-6-(2-methyl-2H-pyrazolo [3,4-b]pyridin-5-yl)thieno [2,3-b]pyridine-2,5-diyl)dimethanol;
(IR)-1-(4,6-bis(1-methyl-1H-pyrazol-5-yl)thieno [2,3-b]pyridin-2-yl)ethanol;
(1S)-1-(4,6-bis(1-methyl-1H-pyrazol-5-yl)thieno[2,3-b]pyridin-2-yl)ethanol;
(1R)-1-(4,6-bis(2-methyl-2H-pyrazolo[3,4-b]pyridin-5-yl)thieno[2,3-b]pyridin-2-yl)ethanol;
(1S)-1-(4,6-bis(2-methyl-2H-pyrazolo[3,4-b]pyridin-5-yl)thieno [2,3-b]pyridin-2-yl)ethanol;
(1R)-1-(4-cyclopropyl-6-(2-methyl-2H-pyrazolo[3,4-b]pyridin-5-yl)thieno[2,3-b]pyridin-2-yl)ethanol;
(1S)-1-(4-cyclopropyl-6-(2-methyl-2H-pyrazolo[3,4-b]pyridin-5-yl)thieno[2,3-b]pyridin-2-yl)ethanol;
(1R)-1-(6-cyclopropyl-4-(2-methyl-2H-pyrazolo[3,4-b]pyridin-5-yl)thieno[2,3-b]pyridin-2-yl)ethanol;
(1S)-1-(6-cyclopropyl-4-(2-methyl-2H-pyrazolo[3,4-b]pyridin-5-yl)thieno[2,3-b]pyridin-2-yl)ethanol;
(1R)-1-(4-cyclopropyl-6-(2-methyl-2H-pyrazolo[3,4-b]pyridin-5-yl)thieno[2,3-b]pyridin-2-yl)-1-propanol;
(1S)-1-(4-cyclopropyl-6-(2-methyl-2H-pyrazolo[3,4-b]pyridin-5-yl)thieno[2,3-b]pyridin-2-yl)-1-propanol;
(R)-(6-(2-methyl-2H-pyrazolo [3,4-b]pyridin-5-yl)thieno [2,3-b]pyridin-2-yl)(tetrahydro-2H-pyran-4-yl)methanol;
(S)-(6-(2-methyl-2H-pyrazolo [3,4-b]pyridin-5-yl)thieno [2,3-b]pyridin-2-yl)(tetrahydro-2H-pyran-4-yl)methanol;
(R)-cyclopropyl(6-(2-methyl-2H-pyrazolo [3,4-b]pyridin-5-yl)thieno [2,3-b]pyridin-2-yl)methanol;
(S)-cyclopropyl(6-(2-methyl-2H-pyrazolo [3,4-b]pyridin-5-yl)thieno [2,3-b]pyridin-2-yl)methanol;
(R)-cyclobutyl(6-(2-methyl-2H-pyrazolo [3,4-b]pyridin-5-yl)thieno [2,3-b]pyridin-2-yl)methanol;
(S)-cyclobutyl(6-(2-methyl-2H-pyrazolo [3,4-b]pyridin-5-yl)thieno [2,3-b]pyridin-2-yl)methanol;
(3R)-3-(6-(2-methyl-2H-pyrazolo[3,4-b]pyridin-5-yl)thieno[2,3-b]pyridin-2-yl)tetrahydro-3-furanol;
(3S)-3-(6-(2-methyl-2H-pyrazolol[3,4-b]pyridin-5-yl)thieno[2,3-b]pyridin-2-yl)tetrahydro-3-furanol;
1-(6-(2-methyl-2H-pyrazolo[3,4-b]pyridin-5-yl)thieno[2,3-b]pyridin-2-yl)cyclobutanol;
4-(6-(2-methyl-2H-pyrazolo[3,4-b]pyridin-5-yl)thieno [2,3-b]pyridin-2-yl)tetrahydro-2H-pyran-4-ol;
(R)-(6-(2-methyl-2H-pyrazolo[3,4-b]pyridin-5-yl)thieno[2,3-b]pyridin-2-yl)((3R)-tetrahydro-3-furanyl)methanol;
(R)-(6-(2-methyl-2H-pyrazolo[3,4-b]pyridin-5-yl)thieno[2,3-b]pyridin-2-yl)((3S)-tetrahydro-3-furanyl)methanol;
(S)-(6-(2-methyl-2H-pyrazolol[3,4-b]pyridin-5-yl)thienol[2,3-b]pyridin-2-yl)((3R)-tetrahydro-3-furanyl)methanol;
(S)-(6-(2-methyl-2H-pyrazolo[3,4-b]pyridin-5-yl)thieno[2,3-b]pyridin-2-yl)((3S)-tetrahydro-3-furanyl)methanol;
3-(6-(2-methyl-2H-pyrazolo[3,4-b]pyridin-5-yl)thieno[2,3-b]pyridin-2-yl)-3-oxetanol;
1-(6-(2-methyl-2H-pyrazolo[3,4-b]pyridin-5-yl)thieno [2,3-b]pyridin-2-yl)cyclopentanol;
(1R)-1-(6-(2-methyl-2H-pyrazolo[3,4-b]pyridin-5-yl)thieno[2,3-b]pyridin-2-yl)-1-propanol;
(1S)-1-(6-(2-methyl-2H-pyrazolo[3,4-b]pyridin-5-yl)thieno[2,3-b]pyridin-2-yl)-1-propanol;
(6-(2-methyl-2H-pyrazolol[3,4-b]pyridin-5-yl)thieno[2,3-b]pyridin-2-yl)methanol;
(1R)-2,2,2-trifluoro-1-(6-(2-methyl-2H-pyrazolo[3,4-b]pyridin-5-yl)thieno[2,3-b]pyridin-2-yl)ethanol;
(1S)-2,2,2-trifluoro-1-(6-(2-methyl-2H-pyrazolo[3,4-b]pyridin-5-yl)thieno[2,3-b]pyridin-2-yl)ethanol;
(1R)-2-methyl-1-(6-(2-methyl-2H-pyrazolo[3,4-b]pyridin-5-yl)thieno[2,3-b]pyridin-2-yl)-1-propanol;
(1S)-2-methyl-1-(6-(2-methyl-2H-pyrazolo[3,4-b]pyridin-5-yl)thieno[2,3-b]pyridin-2-yl)-1-propanol;
(1R)-1-(6-(2-methyl-2H-pyrazolo[3,4-b]pyridin-5-yl)thieno[2,3-b]pyridin-2-yl)ethanol;
(1S)-1-(6-(2-methyl-2H-pyrazolo[3,4-b]pyridin-5-yl)thieno[2,3-b]pyridin-2-yl)ethanol;
(1R)-1-(6-(2-methyl-2H-pyrazolo[3,4-b]pyridin-5-yl)thieno[2,3-b]pyridin-2-yl)-1-butanol;
(1S)-1-(6-(2-methyl-2H-pyrazolo[3,4-b]pyridin-5-yl)thieno[2,3-b]pyridin-2-yl)-1-butanol;
(R)-(6-(2-methyl-2H-pyrazolo[3,4-b]pyridin-5-yl)thieno[2,3-b]pyridin-2-yl)(3-oxetanyl)methanol;
(S)-(6-(2-methyl-2H-pyrazolo[3,4-b]pyridin-5-yl)thieno[2,3-b]pyridin-2-yl)(3-oxetanyl)methanol;
(1R)-3-methoxy-1-(6-(2-methyl-2H-pyrazolo [3,4-b]pyridin-5-yl)thieno[2,3-b]pyridin-2-yl)-1-propanol;
(1S)-3-methoxy-1-(6-(2-methyl-2H-pyrazolo[3,4-b]pyridin-5-yl)thieno[2,3-b]pyridin-2-yl)-1-propanol;
4,4-difluoro-1-(6-(2-methyl-2H-pyrazolo[3,4-b]pyridin-5-yl)thieno[2,3-b]pyridin-2-yl)cyclohexanol;
(2R)-1,1,1-trifluoro-3-methoxy-2-(6-(2-methyl-2H-pyrazolo[3,4-b]pyridin-5-yl)thieno[2,3-b]pyridin-2-yl)-2-propanol;
(2S)-1,1,1-trifluoro-3-methoxy-2-(6-(2-methyl-2H-pyrazolo[3,4-b]pyridin-5-yl)thieno[2,3-b]pyridin-2-yl)-2-propanol;
(1R)-1-cyclopropyl-2,2,2-trifluoro-1-(6-(2-methyl-2H-pyrazolo[3,4-b]pyridin-5-yl)thieno[2,3-b]pyridin-2-yl)ethanol;
(1S)-1-cyclopropyl-2,2,2-trifluoro-1-(6-(2-methyl-2H-pyrazolo[3,4-b]pyridin-5-yl)thieno[2,3-b]pyridin-2-yl)ethanol;
(R)-(3,3-difluorocyclobutyl)(6-(2-methyl-2H-pyrazolo [3,4-b]pyridin-5-yl)thieno[2,3-b]pyridin-2-yl)methanol;
(S)-(3,3-difluorocyclobutyl)(6-(2-methyl-2H-pyrazolo [3,4-b]pyridin-5-yl)thieno [2,3-b]pyridin-2-yl)methanol;
(R)-(4,4-difluorocyclohexyl)(6-(2-methyl-2H-pyrazolo [3,4-b]pyridin-5-yl)thieno [2,3-b]pyridin-2-yl)methanol;
(S)-(4,4-difluorocyclohexyl)(6-(2-methyl-2H-pyrazolo [3,4-b]pyridin-5-yl)thieno [2,3-b]pyridin-2-yl)methanol;
1-methyl-3-(6-(2-methyl-2H-pyrazolo[3,4-b]pyridin-5-yl)thieno[2,3-b]pyridin-2-yl)-3-azetidinol;
(3R)-1-methyl-3-(6-(2-methyl-2H-pyrazolo[3,4-b]pyridin-5-yl)thieno[2,3-b]pyridin-2-yl)-3-piperidinol;
(3S)-1-methyl-3-(6-(2-methyl-2H-pyrazolo[3,4-b]pyridin-5-yl)thieno[2,3-b]pyridin-2-yl)-3-piperidinol;
(2R)-1,1,1-trifluoro-2-(6-(2-methyl-2H-pyrazolo[3,4-b]pyridin-5-yl)thieno[2,3-b]pyridin-2-yl)-2-butanol;
(2S)-1,1,1-trifluoro-2-(6-(2-methyl-2H-pyrazolo[3,4-b]pyridin-5-yl)thieno[2,3-b]pyridin-2-yl)-2-butanol;
(1R)-2-methoxy-1-(6-(2-methyl-2H-pyrazolo[3,4-b]pyridin-5-yl)thieno[2,3-b]pyridin-2-yl)ethanol;
(1S)-2-methoxy-1-(6-(2-methyl-2H-pyrazolo[3,4-b]pyridin-5-yl)thieno[2,3-b]pyridin-2-yl)ethanol;
4-(6-(2-methyl-2H-pyrazolo[3,4-b]pyridin-5-yl)thieno [2,3-b]pyridin-2-yl)tetrahydro-2H-thiopyran-4-ol 1,1-dioxide;
2-(6-(2-methyl-2H-pyrazolo[3,4-b]pyridin-5-yl)thieno [2,3-b]pyridin-2-yl)-2-propanol;
(2R)-2-(6-(2-methyl-2H-pyrazolo[3,4-b]pyridin-5-yl)thieno[2,3-b]pyridin-2-yl)-2-butanol;
(2S)-2-(6-(2-methyl-2H-pyrazolol[3,4-b]pyridin-5-yl)thieno[2,3-b]pyridin-2-yl)-2-butanol;
(1R)-1-cyclopropyl-1-(6-(2-methyl-2H-pyrazolo[3,4-b]pyridin-5-yl)thieno[2,3-b]pyridin-2-yl)ethanol;
(1S)-1-cyclopropyl-1-(6-(2-methyl-2H-pyrazolo[3,4-b]pyridin-5-yl)thieno[2,3-b]pyridin-2-yl)ethanol;
1-methyl-4-(6-(2-methyl-2H-pyrazolo[3,4-b]pyridin-5-yl)thieno[2,3-b]pyridin-2-yl)-4-piperidinol;
(2R)-1-methoxy-2-(6-(2-methyl-2H-pyrazolo [3,4-b]pyridin-5-yl)thieno[2,3-b]pyridin-2-yl)-2-propanol;
(2S)-1-methoxy-2-(6-(2-methyl-2H-pyrazolo[3,4-b]pyridin-5-yl)thieno[2,3-b]pyridin-2-yl)-2-propanol;
3,3-difluoro-1-(6-(2-methyl-2H-pyrazolo[3,4-b]pyridin-5-yl)thieno[2,3-b]pyridin-2-yl)cyclobutanol;
(3R)-1-methyl-3-(6-(2-methyl-2H-pyrazolo[3,4-b]pyridin-5-yl)thieno[2,3-b]pyridin-2-yl)-3-pyrrolidinol;
(3S)-1-methyl-3-(6-(2-methyl-2H-pyrazolo[3,4-b]pyridin-5-yl)thieno[2,3-b]pyridin-2-yl)-3-pyrrolidinol;
(R)-(1-methyl-4-piperidinyl)(6-(2-methyl-2H-pyrazolo[3,4-b]pyridin-5-yl)thieno[2,3-b]pyridin-2-yl)methanol;
(S)-(1-methyl-4-piperidinyl)(6-(2-methyl-2H-pyrazolo[3,4-b]pyridin-5-yl)thieno[2,3-b]pyridin-2-yl)methanol;
(S)-(6-(1-methyl-1H-indazol-5-yl)thieno[2,3-b]pyridin-2-yl)(tetrahydro-2H-pyran-4-yl)methanol;
(R)-(6-(1-methyl-1H-indazol-5-yl)thieno[2,3-b]pyridin-2-yl)(tetrahydro-2H-pyran-4-yl)methanol;
(S)-(6-(1-methyl-1H-pyrazolo[3,4-b]pyridin-5-yl)thieno[2,3-b]pyridin-2-yl)(tetrahydro-2H-pyran-4-yl)methanol;
(R)-(6-(1-methyl-1H-pyrazolo[3,4-b]pyridin-5-yl)thieno[2,3-b]pyridin-2-yl)(tetrahydro-2H-pyran-4-yl)methanol;
(2R)-1,1,1-trifluoro-4-methoxy-2-(6-(2-methyl-2H-pyrazolo[3,4-b]pyridin-5-yl)thieno[2,3-b]pyridin-2-yl)-2-butanol;
(2S)-1,1,1-trifluoro-4-methoxy-2-(6-(2-methyl-2H-pyrazolo[3,4-b]pyridin-5-yl)thieno[2,3-b]pyridin-2-yl)-2-butanol;
(S)-(6-(1H-indazol-5-yl)thienol[2,3-b]pyridin-2-yl)(tetrahydro-2H-pyran-4-yl)methanol;
(R)-(6-(1H-indazol-5-yl)thieno[2,3-b]pyridin-2-yl)(tetrahydro-2H-pyran-4-yl)methanol;
(S)-(6-(6-methyl-1H-indazol-5-yl)thienol[2,3-b]pyridin-2-yl)(tetrahydro-2H-pyran-4-yl)methanol;
(R)-(6-(6-methyl-1H-indazol-5-yl)thienol[2,3-b]pyridin-2-yl)(tetrahydro-2H-pyran-4-yl)methanol;
(2R)-1,1,1-trifluoro-2-(6-(2-methyl-2H-pyrazolo [3,4-b]pyridin-5-yl)thieno[2,3-b]pyridin-2-yl)-2-propanol;
(2S)-1,1,1-trifluoro-2-(6-(2-methyl-2H-pyrazolo[3,4-b]pyridin-5-yl)thieno[2,3-b]pyridin-2-yl)-2-propanol;
(S)-(6-(2-methyl-2H-[1,2,3]triazolo[4,5-b]pyridin-6-yl)thieno[2,3-b]pyridin-2-yl)(tetrahydro-2H-pyran-4-yl)methanol;
(R)-(6-(2-methyl-2H-[1,2,3]triazolo[4,5-b]pyridin-6-yl)thieno[2,3-b]pyridin-2-yl)(tetrahydro-2H-pyran-4-yl)methanol;
(S)-(6-(3-methyl-1H-indazol-5-yl)thieno [2,3-b]pyridin-2-yl)(tetrahydro-2H-pyran-4-yl)methanol;
(R)-(6-(3-methyl-1H-indazol-5-yl)thieno [2,3-b]pyridin-2-yl)(tetrahydro-2H-pyran-4-yl)methanol;
(2R)-4-methoxy-2-(6-(2-methyl-2H-pyrazolo[3,4-b]pyridin-5-yl)thieno[2,3-b]pyridin-2-yl)-2-butanol;
(2S)-4-methoxy-2-(6-(2-methyl-2H-pyrazolo[3,4-b]pyridin-5-yl)thieno[2,3-b]pyridin-2-yl)-2-butanol;
(R)-(6-(3-methyl-3H-[1,2,3]triazolo[4,5-b]pyridin-6-yl)thieno[2,3-b]pyridin-2-yl)(tetrahydro-2H-pyran-4-yl)methanol;
(S)-(6-(3-methyl-3H-[1,2,3]triazolo [4,5-b]pyridin-6-yl)thieno[2,3-b]pyridin-2-yl)(tetrahydro-2H-pyran-4-yl)methanol;
(R)-(1-methyl-3-azetidinyl)(6-(2-methyl-2H-pyrazolo[3,4-b]pyridin-5-yl)thieno[2,3-b]pyridin-2-yl)methanol;
(S)-(1-methyl-3-azetidinyl)(6-(2-methyl-2H-pyrazolo[3,4-b]pyridin-5-yl)thieno[2,3-b]pyridin-2-yl)methanol;
(S)-(6-(1,2-benzoxazol-5-yl)thieno[2,3-b]pyridin-2-yl)(tetrahydro-2H-pyran-4-yl)methanol;
(R)-(6-(1,2-benzoxazol-5-yl)thieno[2,3-b]pyridin-2-yl)(tetrahydro-2H-pyran-4-yl)methanol;
(S)-tetrahydro-2H-pyran-4-yl(6-(3H-[1,2,3]triazolo[4,5-b]pyridin-6-yl)thieno[2,3-b]pyridin-2-yl)methanol;
(R)-tetrahydro-2H-pyran-4-yl(6-(3H-[1,2,3]triazolo[4,5-b]pyridin-6-yl)thieno[2,3-b]pyridin-2-yl)methanol;
(R)-((2R)-1-methyl-2-azetidinyl)(6-(2-methyl-2H-pyrazolo[3,4-b]pyridin-5-yl)thieno[2,3-b]pyridin-2-yl)methanol;
(R)-((2S)-1-methyl-2-azetidinyl)(6-(2-methyl-2H-pyrazolo[3,4-b]pyridin-5-yl)thieno[2,3-b]pyridin-2-yl)methanol;
(S)-((2R)-1-methyl-2-azetidinyl)(6-(2-methyl-2H-pyrazolo[3,4-b]pyridin-5-yl)thieno[2,3-b]pyridin-2-yl)methanol;
(S)-((2S)-1-methyl-2-azetidinyl)(6-(2-methyl-2H-pyrazolo[3,4-b]pyridin-5-yl)thieno[2,3-b]pyridin-2-yl)methanol;
(S)-(3,3-difluorocyclobutyl)(6-(2-methyl-1,3-benzoxazol-5-yl)thieno[2,3-b]pyridin-2-yl)methanol;
(R)-(3,3-difluorocyclobutyl)(6-(2-methyl-1,3-benzoxazol-5-yl)thieno[2,3-b]pyridin-2-yl)methanol;
(S)-(3,3-difluorocyclobutyl)(6-(1H-pyrazolo[3,4-b]pyridin-5-yl)thieno[2,3-b]pyridin-2-yl)methanol;
(R)-(3,3-difluorocyclobutyl)(6-(1H-pyrazolo[3,4-b]pyridin-5-yl)thieno[2,3-b]pyridin-2-yl)methanol;
(S)-(3,3-difluorocyclobutyl)(6-(2-methylimidazo[1,2-a]pyridin-6-yl)thieno[2,3-b]pyridin-2-yl)methanol;
(R)-(3,3-difluorocyclobutyl)(6-(2-methylimidazo[1,2-a]pyridin-6-yl)thieno[2,3-b]pyridin-2-yl)methanol;
6-(2-((R)-(3,3-difluorocyclobutyl)(hydroxy)methyl)thieno [2,3-b]pyridin-6-yl)-3-methyl-4(3H)-pyrimidinone;
6-(2-((S)-(3,3-difluorocyclobutyl)(hydroxy)methyl)thieno[2,3-b]pyridin-6-yl)-3-methyl-4(3H)-pyrimidinone;
6-(2-((S)-(3,3-difluorocyclobutyl)(hydroxy)methyl)thieno[2,3-b]pyridin-6-yl)-2-methyl-1(2H)-isoquinolinone;
6-(2-((R)-(3,3-difluorocyclobutyl)(hydroxy)methyl)thieno[2,3-b]pyridin-6-yl)-2-methyl-1(2H)-isoquinolinone;
(S)-(3,3-difluorocyclobutyl)(6-(2-methyl-1,3-benzoxazol-6-yl)thieno[2,3-b]pyridin-2-yl)methanol;
(R)-(3,3-difluorocyclobutyl)(6-(2-methyl-1,3-benzoxazol-6-yl)thieno[2,3-b]pyridin-2-yl)methanol;
(S)-(3,3-difluorocyclobutyl)(6-(6-quinoxalinyl)thieno [2,3-b]pyridin-2-yl)methanol;
(R)-(3,3-difluorocyclobutyl)(6-(6-quinoxalinyl)thieno [2,3-b]pyridin-2-yl)methanol;
(S)-(3,3-difluorocyclobutyl)(6-(2-ethyl-2H-pyrazolo[3,4-b]pyridin-5-yl)thieno[2,3-b]pyridin-2-yl)methanol;
(R)-(3,3-difluorocyclobutyl)(6-(2-ethyl-2H-pyrazolo [3,4-b]pyridin-5-yl)thieno[2,3-b]pyridin-2-yl)methanol;
(1S)-1-(6-(2-methyl-2H-pyrazolo[3,4-b]pyridin-5-yl)-1-benzothiophen-2-yl)-1-butanol;
(1R)-1-(6-(2-methyl-2H-pyrazolo[3,4-b]pyridin-5-yl)-1-benzothiophen-2-yl)-1-butanol;
(S)-(3,3-difluorocyclobutyl)(6-(2-methylimidazo[1,2-a]pyridin-7-yl)thieno[2,3-b]pyridin-2-yl)methanol;
(R)-(3,3-difluorocyclobutyl)(6-(2-methylimidazo[1,2-a]pyridin-7-yl)thieno[2,3-b]pyridin-2-yl)methanol;
(S)-(3,3-difluorocyclobutyl)(6-(2-(2-propanyl)-2H-indazol-5-yl)thieno[2,3-b]pyridin-2-yl)methanol;
(R)-(3,3-difluorocyclobutyl)(6-(2-(2-propanyl)-2H-indazol-5-yl)thieno [2,3-b]pyridin-2-yl)methanol;
(S)-(3,3-difluorocyclobutyl)(6-(5-pyrimidinyl)thieno[2,3-b]pyridin-2-yl)methanol;
(R)-(3,3-difluorocyclobutyl)(6-(5-pyrimidinyl)thieno[2,3-b]pyridin-2-yl)methanol;
(S)-cyclopentyl(6-(2-methyl-2H-pyrazolo[3,4-b]pyridin-5-yl)-1-benzothiophen-2-yl)methanol;
(R)-cyclopentyl(6-(2-methyl-2H-pyrazolo[3,4-b]pyridin-5-yl)-1-benzothiophen-2-yl)methanol;
(1S)-3-methoxy-1-(6-(2-methyl-2H-pyrazolo1[3,4-b]pyridin-5-yl)-1-benzothiophen-2-yl)-1-propanol;
(1R)-3-methoxy-1-(6-(2-methyl-2H-pyrazolo[3,4-b]pyridin-5-yl)-1-benzothiophen-2-yl)-1-propanol;
(1S)-4,4,4-trifluoro-1-(6-(2-methyl-2H-pyrazolo[3,4-b]pyridin-5-yl)-1-benzothiophen-2-yl)-1-butanol;
(1R)-4,4,4-trifluoro-1-(6-(2-methyl-2H-pyrazolo[3,4-b]pyridin-5-yl)-1-benzothiophen-2-yl)-1-butanol;
(S)-(3,3-difluorocyclobutyl)(6-(2-methyl-2H-pyrazolo[3,4-b]pyridin-5-yl)-1-benzothiophen-2-yl)methanol;
(R)-(3,3-difluorocyclobutyl)(6-(2-methyl-2H-pyrazolo[3,4-b]pyridin-5-yl)-1-benzothiophen-2-yl)methanol;
(R)-(6-(2-methyl-2H-pyrazolo[3,4-b]pyridin-5-yl)thieno[2,3-b]pyridin-2-yl)((3R)-1-methyl-3-pyrrolidinyl)methanol;
(R)-(6-(2-methyl-2H-pyrazolo[3,4-b]pyridin-5-yl)thieno[2,3-b]pyridin-2-yl)((3S)-1-methyl-3-pyrrolidinyl)methanol;
(S)-(6-(2-methyl-2H-pyrazolo[3,4-b]pyridin-5-yl)thieno[2,3-b]pyridin-2-yl)((3R)-1-methyl-3-pyrrolidinyl)methanol;
(S)-(6-(2-methyl-2H-pyrazolo[3,4-b]pyridin-5-yl)thieno[2,3-b]pyridin-2-yl)((3S)-1-methyl-3-pyrrolidinyl)methanol;
(R)-((3R)-1-methyl-3-piperidinyl)(6-(2-methyl-2H-pyrazolo[3,4-b]pyridin-5-yl)thieno[2,3-b]pyridin-2-yl)methanol;
(R)-((3S)-1-methyl-3-piperidinyl)(6-(2-methyl-2H-pyrazolo[3,4-b]pyridin-5-yl)thieno[2,3-b]pyridin-2-yl)methanol;
(S)-((3R)-1-methyl-3-piperidinyl)(6-(2-methyl-2H-pyrazolo[3,4-b]pyridin-5-yl)thieno[2,3-b]pyridin-2-yl)methanol;
(S)-((3S)-1-methyl-3-piperidinyl)(6-(2-methyl-2H-pyrazolo[3,4-b]pyridin-5-yl)thieno[2,3-b]pyridin-2-yl)methanol;
(S)-(3,3-difluorocyclobutyl)(6-(2-(2-propanyl)-2H-pyrazolo[3,4-b]pyridin-5-yl)thieno[2,3-b]pyridin-2-yl)methanol;
(R)-(3,3-difluorocyclobutyl)(6-(2-(2-propanyl)-2H-pyrazolo[3,4-b]pyridin-5-yl)thieno[2,3-b]pyridin-2-yl)methanol;
(S)-(3,3-difluorocyclobutyl)(6-(1-(2-propanyl)-1H-pyrazolo[3,4-b]pyridin-5-yl)thieno[2,3-b]pyridin-2-yl)methanol;
(R)-(3,3-difluorocyclobutyl)(6-(1-(2-propanyl)-1H-pyrazolo[3,4-b]pyridin-5-yl)thieno[2,3-b]pyridin-2-yl)methanol;
(R)-(6-(2-methyl-2H-pyrazolo [3,4-b]pyridin-5-yl)thieno [2,3-b]pyridin-2-yl)(tetrahydro-2H-pyran-4-yl)methan-d-ol;
(S)-(6-(2-methyl-2H-pyrazolo [3,4-b]pyridin-5-yl)thieno [2,3-b]pyridin-2-yl)(tetrahydro-2H-pyran-4-yl)methan-d-ol;
(R)-(3-amino-6-(5-pyrimidinyl)thieno [2,3-b]pyridin-2-yl)(3,3-difluorocyclobutyl)methanol;
(S)-(3-amino-6-(5-pyrimidinyl)thieno [2,3-b]pyridin-2-yl)(3,3-difluorocyclobutyl)methanol;
2-(6-(2-methyl-2H-pyrazolo[3,4-b]pyridin-5-yl)thieno [2,3-b]pyridin-2-yl)spiro[3.3]heptan-2-ol;
3,3-dimethyl-1-(6-(2-methyl-2H-pyrazolo[3,4-b]pyridin-5-yl)thieno[2,3-b]pyridin-2-yl)cyclobutanol;
6,6-difluoro-2-(6-(2-methyl-2H-pyrazolo[3,4-b]pyridin-5-yl)thieno[2,3-b]pyridin-2-yl)spiro[3.3]heptan-2-ol;
6-(6-(2-methyl-2H-pyrazolo[3,4-b]pyridin-5-yl)thieno [2,3-b]pyridin-2-yl)-2-oxaspiro[3.3]heptan-6-ol;
cis-1-(6-(2-methyl-2H-pyrazolo[3,4-b]pyridin-5-yl)thieno[2,3-b]pyridin-2-yl)-3-(trifluoromethyl)cyclobutanol;
cis-3-(difluoromethyl)-1-(6-(2-methyl-2H-pyrazolo[3,4-b]pyridin-5-yl)thieno[2,3-b]pyridin-2-yl)cyclobutanol;
cis-3-methoxy-1-(6-(2-methyl-2H-pyrazolo[3,4-b]pyridin-5-yl)thieno[2,3-b]pyridin-2-yl)cyclobutanol;
(S)-(3,3-difluorocyclobutyl)(5-(2-methyl-2H-pyrazolo[3,4-b]pyridin-5-yl)[1,3]thiazolo[5,4-b]pyridin-2-yl)methanol;
(R)-(3,3-difluorocyclobutyl)(5-(2-methyl-2H-pyrazolo[3,4-b]pyridin-5-yl)[1,3]thiazolo[5,4-b]pyridin-2-yl)methanol;
1-(6-(2-ethyl-2H-pyrazolo[3,4-b]pyridin-5-yl)thieno[2,3-b]pyridin-2-yl)-3,3-difluorocyclobutanol;
(S)-(3,3-difluorocyclobutyl)(5-(2-methyl-2H-pyrazolo[3,4-b]pyridin-5-yl)thieno [3,2-b]pyridin-2-yl)methanol;
(R)-(3,3-difluorocyclobutyl)(5-(2-methyl-2H-pyrazolo [3,4-b]pyridin-5-yl)thieno[3,2-b]pyridin-2-yl)methanol;
3,3-difluoro-1-(6-(3-methyl-3H-imidazo[4,5-b]pyridin-6-yl)thieno[2,3-b]pyridin-2-yl)cyclobutanol;
3,3-difluoro-1-(6-(2-methyl[1,3]oxazolo[4,5-b]pyridin-6-yl)thieno[2,3-b]pyridin-2-yl)cyclobutanol;
3,3-difluoro-1-(6-(2-methyl-3H-imidazo[4,5-b]pyridin-6-yl)thieno[2,3-b]pyridin-2-yl)cyclobutanol;
3,3-difluoro-1-(6-(1H-pyrazolo[3,4-b]pyridin-5-yl)thieno[2,3-b]pyridin-2-yl)cyclobutanol;
3,3-difluoro-1-(6-(2-methylimidazo[1,2-a]pyridin-6-yl)thieno[2,3-b]pyridin-2-yl)cyclobutanol;
1-(6-(2-amino-5-pyrimidinyl)thieno[2,3-b]pyridin-2-yl)-3,3-difluorocyclobutanol;
(S)-(3,3-difluorocyclobutyl)(5-(2-methyl-2H-pyrazolo[3,4-b]pyridin-5-yl)[1,3]thiazolo[4,5-b]pyridin-2-yl)methanol;
(R)-(3,3-difluorocyclobutyl)(5-(2-methyl-2H-pyrazolo[3,4-b]pyridin-5-yl)[1,3]thiazolo[4,5-b]pyridin-2-yl)methanol;
3,3-difluoro-1-(6-(2-methyl-1,3-benzoxazol-6-yl)thieno[2,3-b]pyridin-2-yl)cyclobutanol;
trans-3-fluoro-1-(6-(2-methyl-2H-pyrazolo[3,4-b]pyridin-5-yl)thieno[2,3-b]pyridin-2-yl)cyclobutanol;
(R)-(3,3-difluorocyclobutyl)(2-(2-methyl-2H-pyrazolo[3,4-b]pyridin-5-yl)thieno[2,3-d]pyrimidin-6-yl)methanol;
(S)-(3,3-difluorocyclobutyl)(2-(2-methyl-2H-pyrazolo[3,4-b]pyridin-5-yl)thieno[2,3-d]pyrimidin-6-yl)methanol;
(S)-(3-chloro-6-(2-methyl-2H-pyrazolo[3,4-b]pyridin-5-yl)thieno[2,3-b]pyridin-2-yl)(3,3-difluorocyclobutyl)methanol;
(R)-(3-chloro-6-(2-methyl-2H-pyrazolo[3,4-b]pyridin-5-yl)thieno[2,3-b]pyridin-2-yl)(3,3-difluorocyclobutyl)methanol;
3,3-difluoro-1-(6-(3H-imidazo[4,5-b]pyridin-6-yl)thieno[2,3-b]pyridin-2-yl)cyclobutanol;
3,3-difluoro-1-(6-(1H-indazol-5-yl)thieno[2,3-b]pyridin-2-yl)cyclobutanol;
(R)-(3,3-difluorocyclobutyl)(5-(2-methyl-2H-pyrazolo [3,4-b]pyridin-5-yl)thieno[2,3-b]pyridin-2-yl)methanol;
(S)-(3,3-difluorocyclobutyl)(5-(2-methyl-2H-pyrazolo [3,4-b]pyridin-5-yl)thieno [2,3-b]pyridin-2-yl)methanol;
(S)-(3,3-difluorocyclobutyl)(6-(2-methyl-2H-pyrazolo [3,4-b]pyridin-5-yl)thieno [3,2-b]pyridin-2-yl)methanol;
(R)-(3,3-difluorocyclobutyl)(6-(2-methyl-2H-pyrazolo [3,4-b]pyridin-5-yl)thieno[3,2-b]pyridin-2-yl)methanol;
3,3-difluoro-1-(6-(2-(methylamino)-5-pyrimidinyl)thieno[2,3-b]pyridin-2-yl)cyclobutanol;
(R)-(3,3-difluorocyclobutyl)(6-(2-methyl-2H-pyrazolo[3,4-b]pyridin-5-yl)thieno[2,3-b]pyridin-2-yl)methan-d-ol;
(S)-(3,3-difluorocyclobutyl)(6-(2-methyl-2H-pyrazolo [3,4-b]pyridin-5-yl)thieno [2,3-b]pyridin-2-yl)methan-d-ol;
3,3-difluoro-1-(6-(2-methylimidazo[1,2-a]pyridin-7-yl)thieno[2,3-b]pyridin-2-yl)cyclobutanol;
3,3-difluoro-1-(6-(2-methylimidazo[1,2-a]pyrimidin-6-yl)thieno[2,3-b]pyridin-2-yl)cyclobutanol;
(2R)-2-(3,3-difluorocyclobutyl)-2-(6-(2-methyl-2H-pyrazolo [3,4-b]pyridin-5-yl)thieno[2,3-b]pyridin-2-yl)ethanol;
(2S)-2-(3,3-difluorocyclobutyl)-2-(6-(2-methyl-2H-pyrazolo [3,4-b]pyridin-5-yl)thieno [2,3-b]pyridin-2-yl)ethanol;
3,3-difluoro-1-(6-(5-pyrimidinyl)thieno[2,3-b]pyridin-2-yl)cyclobutanol;
3,3-difluoro-1-(6-(pyrido[2,3-b]pyrazin-7-yl)thieno[2,3-b]pyridin-2-yl)cyclobutanol;
(S)-(3,3-difluorocyclobutyl)(2-(2-methyl-2H-pyrazolo [3,4-b]pyridin-5-yl)-7-quinolinyl)methanol;
(R)-(3,3-difluorocyclobutyl)(2-(2-methyl-2H-pyrazolo [3,4-b]pyridin-5-yl)-7-quinolinyl)methanol;
3,3-difluoro-1-(6-(2-methyl-2H-pyrazolo[3,4-b]pyridin-5-yl)-1-benzothiophen-2-yl)cyclobutanol;
3,3-difluoro-1-(6-(2-methyl-2H-indazol-5-yl)thieno[2,3-b]pyridin-2-yl)cyclobutanol;
1-(6-(1,2-benzoxazol-5-yl)thieno[2,3-b]pyridin-2-yl)-3,3-difluorocyclobutanol;
3,3-difluoro-1-(6-(3-methyl-3H-[1,2,3]triazolo[4,5-b]pyridin-6-yl)thieno[2,3-b]pyridin-2-yl)cyclobutanol;
2-(6-(2-methyl-2H-pyrazolo[3,4-b]pyridin-5-yl)thieno[2,3-b]pyridin-2-yl)spiro[3.5]nonan-2-ol;
2-(6-(2-methyl-2H-pyrazolo[3,4-b]pyridin-5-yl)thieno[2,3-b]pyridin-2-yl)spiro[3.4]octan-2-ol;
3,3-difluoro-1-(6-(2-methylimidazo[1,2-b]pyridazin-7-yl)thieno[2,3-b]pyridin-2-yl)cyclobutanol;
3,3-difluoro-1-(6-(3H-[1,2,3]triazolo[4,5-b]pyridin-6-yl)thieno[2,3-b]pyridin-2-yl)cyclobutanol;
(R)-(3,3-difluorocyclobutyl)(6-(3-methyl-3H-[1,2,3]triazolo[4,5-b]pyridin-6-yl)thieno[2,3-b]pyridin-2-yl)methanol;
(S)-(3,3-difluorocyclobutyl)(6-(3-methyl-3H-[1,2,3]triazolo[4,5-b]pyridin-6-yl)thieno[2,3-b]pyridin-2-yl)methanol;
trans-6-(6-(2-methyl-2H-pyrazolo[3,4-b]pyridin-5-yl)thieno[2,3-b]pyridin-2-yl)-1-oxaspiro[3.3]heptan-6-ol;
cis-6-(6-(2-methyl-2H-pyrazolo[3,4-b]pyridin-5-yl)thieno[2,3-b]pyridin-2-yl)-1-oxaspiro[3.3]heptan-6-ol;
3,3-dimethyl-1-(2-(2-methyl-2H-pyrazolo[3,4-b]pyridin-5-yl)thieno[2,3-d]pyrimidin-6-yl)cyclobutanol;
cis-3-(methoxymethyl)-1-(6-(2-methyl-2H-pyrazolo[3,4-b]pyridin-5-yl)thieno[2,3-b]pyridin-2-yl)cyclobutanol;
trans-3-methoxy-3-methyl-1-(6-(2-methyl-2H-pyrazolo[3,4-b]pyridin-5-yl)thieno[2,3-b]pyridin-2-yl)cyclobutanol;
cis-3-methoxy-3-methyl-1-(6-(2-methyl-2H-pyrazolo[3,4-b]pyridin-5-yl)thieno[2,3-b]pyridin-2-yl)cyclobutanol;
(R)-(3,3-difluorocyclobutyl)(3-methyl-6-(2-methyl-2H-pyrazolo[3,4-b]pyridin-5-yl)thieno[2,3-b]pyridin-2-yl)methanol;
(S)-(3,3-difluorocyclobutyl)(3-methyl-6-(2-methyl-2H-pyrazolo [3,4-b]pyridin-5-yl)thieno [2,3-b]pyridin-2-yl)methanol;
1-(6-(2-methyl-2H-pyrazolo [3,4-b]pyridin-5-yl)thieno [2,3-b]pyridin-2-yl)cyclopropanol;
5-(6-(2-methyl-2H-pyrazolo[3,4-b]pyridin-5-yl)thieno[2,3-b]pyridin-2-yl)spiro[2.3]hexan-5-ol;
cis-3-cyclopropyl-1-(6-(2-methyl-2H-pyrazolo[3,4-b]pyridin-5-yl)thieno[2,3-b]pyridin-2-yl)cyclobutanol;
trans-3-hydroxy-1-methyl-3-(6-(2-methyl-2H-pyrazolo[3,4-b]pyridin-5-yl)thieno[2,3-b]pyridin-2-yl)cyclobutanecarbonitrile;
cis-3-hydroxy-1-methyl-3-(6-(2-methyl-2H-pyrazolo[3,4-b]pyridin-5-yl)thieno[2,3-b]pyridin-2-yl)cyclobutanecarbonitrile;
(1S,2R)-2-methyl-1-(6-(2-methyl-2H-pyrazolo[3,4-b]pyridin-5-yl)thieno[2,3-b]pyridin-2-yl)cyclobutanol;
(1R,2S)-2-methyl-1-(6-(2-methyl-2H-pyrazolo[3,4-b]pyridin-5-yl)thieno[2,3-b]pyridin-2-yl)cyclobutanol;
(1S)-2,2-dimethyl-1-(6-(2-methyl-2H-pyrazolo[3,4-b]pyridin-5-yl)thieno[2,3-b]pyridin-2-yl)cyclobutanol;
(1R)-2,2-dimethyl-1-(6-(2-methyl-2H-pyrazolo[3,4-b]pyridin-5-yl)thieno[2,3-b]pyridin-2-yl)cyclobutanol;
trans-3-methoxy-1-(6-(2-methyl-2H-pyrazolo[3,4-b]pyridin-5-yl)thieno[2,3-b]pyridin-2-yl)-3-(trifluoromethyl)cyclobutanol;
cis-3-methoxy-1-(6-(2-methyl-2H-pyrazolo[3,4-b]pyridin-5-yl)thieno[2,3-b]pyridin-2-yl)-3-(trifluoromethyl)cyclobutanol;
cis-1-(6-(2-methyl-2H-pyrazolo[3,4-b]pyridin-5-yl)thieno[2,3-b]pyridin-2-yl)-3-(trifluoromethoxy)cyclobutanol;
3,3-dimethyl-1-(5-(2-methyl-2H-pyrazolo[3,4-b]pyridin-5-yl)[1,3]thiazolo[5,4-b]pyridin-2-yl)cyclobutanol;
cis-1-(3-chloro-6-(2-methyl-2H-pyrazolo[3,4-b]pyridin-5-yl)thieno[2,3-b]pyridin-2-yl)-3-methoxycyclobutanol;
(1R,2R)-2-methoxy-1-(6-(2-methyl-2H-pyrazolo[3,4-b]pyridin-5-yl)thieno[2,3-b]pyridin-2-yl)cyclobutanol;
(1R,2S)-2-methoxy-1-(6-(2-methyl-2H-pyrazolo[3,4-b]pyridin-5-yl)thieno[2,3-b]pyridin-2-yl)cyclobutanol;
(1S,2R)-2-methoxy-1-(6-(2-methyl-2H-pyrazolo[3,4-b]pyridin-5-yl)thieno[2,3-b]pyridin-2-yl)cyclobutanol;
cis-1-(2-(2-methyl-2H-pyrazolo[3,4-b]pyridin-5-yl)thieno[2,3-d]pyrimidin-6-yl)-3-(trifluoromethyl)cyclobutanol;
(1S,2S)-2-methoxy-1-(6-(2-methyl-2H-pyrazolo[3,4-b]pyridin-5-yl)thieno[2,3-b]pyridin-2-yl)cyclobutanol;
1-(3-chloro-6-(2-methyl-2H-pyrazolo[3,4-b]pyridin-5-yl)thieno[2,3-b]pyridin-2-yl)-3,3-dimethylcyclobutanol;
cis-1-(5-(2-methyl-2H-pyrazolo[3,4-b]pyridin-5-yl)[1,3]thiazolo[5,4-b]pyridin-2-yl)-3-(trifluoromethyl)cyclobutanol;
(S)-(6-(2-methyl-2H-pyrazolo[3,4-b]pyridin-5-yl)thieno[2,3-b]pyridin-2-yl)((cis)-3-(trifluoromethyl)cyclobutyl)methanol;
(R)-(6-(2-methyl-2H-pyrazolo[3,4-b]pyridin-5-yl)thieno[2,3-b]pyridin-2-yl)((cis)-3-(trifluoromethyl)cyclobutyl)methanol;
(S)-(6-(2-methyl-2H-pyrazolo[3,4-b]pyridin-5-yl)thieno[2,3-b]pyridin-2-yl)((trans)-3-(trifluoromethyl)cyclobutyl)methanol;
(R)-(6-(2-methyl-2H-pyrazolo[3,4-b]pyridin-5-yl)thieno[2,3-b]pyridin-2-yl)((trans)-3-(trifluoromethyl)cyclobutyl)methanol;
cis-1-(3-chloro-6-(2-methyl-2H-pyrazolo[3,4-b]pyridin-5-yl)thieno[2,3-b]pyridin-2-yl)-3-(trifluoromethyl)cyclobutanol;
(R)-((1R)-2,2-difluorocyclopropyl)(6-(2-methyl-2H-pyrazolo[3,4-b]pyridin-5-yl)thieno[2,3-b]pyridin-2-yl)methanol;
(S)-((1S)-2,2-difluorocyclopropyl)(6-(2-methyl-2H-pyrazolo[3,4-b]pyridin-5-yl)thieno[2,3-b]pyridin-2-yl)methanol;
(S)-((1R)-2,2-difluorocyclopropyl)(6-(2-methyl-2H-pyrazolo[3,4-b]pyridin-5-yl)thieno[2,3-b]pyridin-2-yl)methanol;
(R)-((1S)-2,2-difluorocyclopropyl)(6-(2-methyl-2H-pyrazolo[3,4-b]pyridin-5-yl)thieno[2,3-b]pyridin-2-yl)methanol;
(R)-(3,3-difluoro-1-methylcyclobutyl)(6-(2-methyl-2H-pyrazolo[3,4-b]pyridin-5-yl)thieno[2,3-b]pyridin-2-yl)methanol;
(S)-(3,3-difluoro-1-methylcyclobutyl)(6-(2-methyl-2H-pyrazolo[3,4-b]pyridin-5-yl)thieno[2,3-b]pyridin-2-yl)methanol;
(R)-bicyclo[1.1.1]pentan-1-yl(6-(2-methyl-2H-pyrazolo[3,4-b]pyridin-5-yl)thieno[2,3-b]pyridin-2-yl)methanol;
(S)-bicyclo[1.1.1]pentan-1-yl(6-(2-methyl-2H-pyrazolo[3,4-b]pyridin-5-yl)thieno[2,3-b]pyridin-2-yl)methanol;
(S)-((cis)-3-methoxycyclobutyl)(6-(2-methyl-2H-pyrazolo[3,4-b]pyridin-5-yl)thieno[2,3-b]pyridin-2-yl)methanol;
cis-3-(difluoromethyl)-1-(6-(3-methyl-3H-[1,2,3]triazolo[4,5-b]pyridin-6-yl)thieno[2,3-b]pyridin-2-yl)cyclobutanol;
(R)-((cis)-3-methoxycyclobutyl)(6-(2-methyl-2H-pyrazolo[3,4-b]pyridin-5-yl)thieno[2,3-b]pyridin-2-yl)methanol;
(S)-((trans)-3-methoxycyclobutyl)(6-(2-methyl-2H-pyrazolo[3,4-b]pyridin-5-yl)thieno[2,3-b]pyridin-2-yl)methanol;
(R)-((trans)-3-methoxycyclobutyl)(6-(2-methyl-2H-pyrazolo[3,4-b]pyridin-5-yl)thieno[2,3-b]pyridin-2-yl)methanol;
3,3-dimethyl-1-(2-(2-methyl-2H-pyrazolo[3,4-b]pyridin-5-yl)-7-quinolinyl)cyclobutanol;
3,3-difluoro-1-(3-methyl-6-(2-methyl-2H-pyrazolo[3,4-b]pyridin-5-yl)thieno[2,3-b]pyridin-2-yl)cyclobutanol;
cis-1-(3-methyl-6-(2-methyl-2H-pyrazolo[3,4-b]pyridin-5-yl)thieno[2,3-b]pyridin-2-yl)-3-(trifluoromethyl)cyclobutanol;
cis-1-(6-(3-methyl-3H-[1,2,3]triazolo[4,5-b]pyridin-6-yl)thieno[2,3-b]pyridin-2-yl)-3-(trifluoromethyl)cyclobutanol;
cis-3-(difluoromethyl)-1-(2-(2-methyl-2H-pyrazolo[3,4-b]pyridin-5-yl)thieno[2,3-d]pyrimidin-6-yl)cyclobutanol;
(R)-(2-(3-methyl-3H-[1,2,3]triazolo [4,5-b]pyridin-6-yl)-7-quinolinyl)(tetrahydro-2H-pyran-4-yl)methanol;
(S)-(2-(3-methyl-3H-[1,2,3]triazolo[4,5-b]pyridin-6-yl)-7-quinolinyl)(tetrahydro-2H-pyran-4-yl)methanol;
(R)-(2-(2-methyl-2H-pyrazolo [3,4-b]pyridin-5-yl)-7-quinolinyl)(tetrahydro-2H-pyran-4-yl)methanol;
(S)-(2-(2-methyl-2H-pyrazolo [3,4-b]pyridin-5-yl)-7-quinolinyl)(tetrahydro-2H-pyran-4-yl)methanol;
cis-3-(difluoromethyl)-1-(5-(2-methyl-2H-pyrazolo[3,4-b]pyridin-5-yl)[1,3]thiazolo[5,4-b]pyridin-2-yl)cyclobutanol;
cis-3-(difluoromethyl)-1-(5-(3-methyl-3H-[1,2,3]triazolo[4,5-b]pyridin-6-yl)[1,3]thiazolo[5,4-b]pyridin-2-yl)cyclobutanol;
(R)-(3-methyl-6-(3-methyl-3H-[1,2,3]triazolo[4,5-b]pyridin-6-yl)thieno[2,3-b]pyridin-2-yl)(tetrahydro-2H-pyran-4-yl)methanol;
(S)-(3-methyl-6-(3-methyl-3H-[1,2,3]triazolo[4,5-b]pyridin-6-yl)thieno[2,3-b]pyridin-2-yl)(tetrahydro-2H-pyran-4-yl)methanol;
(R)-(3-methyl-6-(2-methyl-2H-pyrazolo [3,4-b]pyridin-5-yl)thieno1[2,3-b]pyridin-2-yl)(tetrahydro-2H-pyran-4-yl)methanol;
(S)-(3-methyl-6-(2-methyl-2H-pyrazolo [3,4-b]pyridin-5-yl)thieno1[2,3-b]pyridin-2-yl)(tetrahydro-2H-pyran-4-yl)methanol;
cis-3-(difluoromethyl)-1-(2-(2-methyl-2H-pyrazolo[3,4-b]pyridin-5-yl)-7-quinolinyl)cyclobutanol;
(R)-(6-(2-methyl-2H-pyrazolo[3,4-b]pyridin-5-yl)thieno[2,3-b]pyridin-2-yl)(3-(trifluoromethyl)bicyclo[1.1.1]pentan-1-yl)methanol;
(S)-(6-(2-methyl-2H-pyrazolo[3,4-b]pyridin-5-yl)thieno[2,3-b]pyridin-2-yl)(3-(trifluoromethyl)bicyclo[1.1.1]pentan-1-yl)methanol;
cis-3-fluoro-1-(6-(2-methyl-2H-pyrazolo[3,4-b]pyridin-5-yl)thieno[2,3-b]pyridin-2-yl)cyclobutanol;
cis-3-(difluoromethyl)-1-(3-methyl-6-(2-methyl-2H-pyrazolo[3,4-b]pyridin-5-yl)thieno[2,3-b]pyridin-2-yl)cyclobutanol;
cis-3-(difluoromethyl)-1-(2-(2-methyl-2H-pyrazolo[3,4-b]pyridin-5-yl)-6-quinolinyl)cyclobutanol;
cis-3-(difluoromethyl)-1-(5-(2-methyl-2H-pyrazolo[3,4-b]pyridin-5-yl)thieno[3,2-b]pyridin-2-yl)cyclobutanol;
cis-3-(difluoromethyl)-1-(2-(3-methyl-3H-[1,2,3]triazolo[4,5-b]pyridin-6-yl)thieno[2,3-d]pyrimidin-6-yl)cyclobutanol;
cis-1-(5-(2-methyl-2H-pyrazolo[3,4-b]pyridin-5-yl)thieno[3,2-b]pyridin-2-yl)-3-(trifluoromethyl)cyclobutanol;
(S)-(3,3-difluorocyclobutyl)(2-(2-methyl-2H-pyrazolo [3,4-b]pyridin-5-yl)-6-quinolinyl)methanol;
(R)-(3,3-difluorocyclobutyl)(2-(2-methyl-2H-pyrazolo [3,4-b]pyridin-5-yl)-6-quinolinyl)methanol;
cis-3-(difluoromethyl)-1-(6-(2-ethyl-2H-pyrazolo[3,4-b]pyridin-5-yl)thieno[2,3-b]pyridin-2-yl)cyclobutanol;
(trans)-1,1-difluoro-5-(6-(2-methyl-2H-pyrazolo[3,4-b]pyridin-5-yl)thieno[2,3-b]pyridin-2-yl)spiro1[2.3]hexan-5-ol;
(cis)-1,1-difluoro-5-(6-(2-methyl-2H-pyrazolo[3,4-b]pyridin-5-yl)thieno[2,3-b]pyridin-2-yl)spiro1[2.3]hexan-5-ol;
cis-3-(difluoromethyl)-1-(2-(2-methyl-2H-pyrazolo[3,4-b]pyridin-5-yl)thieno[3,2-d]pyrimidin-6-yl)cyclobutanol;
cis-3-(difluoromethyl)-1-(5-methyl-2-(2-methyl-2H-pyrazolo[3,4-b]pyridin-5-yl)thieno[2,3-d]pyrimidin-6-yl)cyclobutanol;
trans-3-hydroxy-1-methyl-3-(5-(2-methyl-2H-pyrazolo[3,4-b]pyridin-5-yl)[1,3]thiazolo[5,4-b]pyridin-2-yl)cyclobutanecarbonitrile;
cis-3-hydroxy-1-methyl-3-(5-(2-methyl-2H-pyrazolo[3,4-b]pyridin-5-yl)[1,3]thiazolo[5,4-b]pyridin-2-yl)cyclobutanecarbonitrile;
cis-3-(difluoromethyl)-1-(6-(2-methylimidazo[1,2-b]pyridazin-7-yl)thieno[2,3-b]pyridin-2-yl)cyclobutanol;
cis-3-(difluoromethyl)-1-(6-(2-methyl-3H-imidazo[4,5-b]pyridin-6-yl)thieno[2,3-b]pyridin-2-yl)cyclobutanol;
(R)-(3,3-difluorocyclobutyl)(3-(2-methyl-2H-pyrazolo[3,4-b]pyridin-5-yl)thieno[2,3-b]pyrazin-6-yl)methanol;
(S)-(3,3-difluorocyclobutyl)(3-(2-methyl-2H-pyrazolo [3,4-b]pyridin-5-yl)thieno[2,3-b]pyrazin-6-yl)methanol;
(2S)-2-(3,3-difluorocyclobutyl)-2-(3-methyl-6-(2-methyl-2H-pyrazolo[3,4-b]pyridin-5-yl)thienol[2,3-b]pyridin-2-yl)ethanol;
(2R)-2-(3,3-difluorocyclobutyl)-2-(3-methyl-6-(2-methyl-2H-pyrazolo[3,4-b]pyridin-5-yl)thienol[2,3-b]pyridin-2-yl)ethanol;
cis-3-methoxy-1-(2-(2-methyl-2H-pyrazolo[3,4-b]pyridin-5-yl)thieno[2,3-d]pyrimidin-6-yl)cyclobutanol;
cis-1-(2-(3-methyl-3H-[1,2,3]triazolo[4,5-b]pyridin-6-yl)thieno[2,3-d]pyrimidin-6-yl)-3-(trifluoromethyl)cyclobutanol;
cis-3-hydroxy-1-methyl-3-(2-(2-methyl-2H-pyrazolo [3,4-b]pyridin-5-yl)thieno[2,3-d]pyrimidin-6-yl)cyclobutanecarbonitrile;
trans-3-hydroxy-1-methyl-3-(2-(2-methyl-2H-pyrazolo[3,4-b]pyridin-5-yl)thieno[2,3-d]pyrimidin-6-yl)cyclobutanecarbonitrile;
cis-3-(difluoromethyl)-1-(6-(2-methylimidazo[1,2-a]pyrimidin-6-yl)thieno[2,3-b]pyridin-2-yl)cyclobutanol;
trans-3-hydroxy-1-methyl-3-(2-(3-methyl-3H-[1,2,3]triazolo[4,5-b]pyridin-6-yl)thieno[2,3-d]pyrimidin-6-yl)cyclobutanecarbonitrile;
cis-3-hydroxy-1-methyl-3-(2-(3-methyl-3H-[1,2,3]triazolo[4,5-b]pyridin-6-yl)thieno[2,3-d]pyrimidin-6-yl)cyclobutanecarbonitrile;
cis-3-cyclopropyl-1-(2-(2-methyl-2H-pyrazolo[3,4-b]pyridin-5-yl)thieno[2,3-d]pyrimidin-6-yl)cyclobutanol;
cis-1-(2-(2-methyl-2H-pyrazolo[3,4-b]pyridin-5-yl)thieno[2,3-d]pyrimidin-6-yl)-3-(trifluoromethoxy)cyclobutanol;
cis-1-(6-(3-methyl-3H-[1,2,3]triazolo[4,5-b]pyridin-6-yl)thieno[2,3-b]pyridin-2-yl)-3-(trifluoromethoxy)cyclobutanol;
cis-3-methoxy-3-methyl-1-(6-(3-methyl-3H-[1,2,3]triazolo[4,5-b]pyridin-6-yl)thieno[2,3-b]pyridin-2-yl)cyclobutanol;
cis-3-(difluoromethyl)-1-(6-(3H-[1,2,3]triazolo[4,5-b]pyridin-6-yl)thieno[2,3-b]pyridin-2-yl)cyclobutanol;
trans-3-hydroxy-1-methyl-3-(6-(3-methyl-3H-[1,2,3]triazolo[4,5-b]pyridin-6-yl)thieno[2,3-b]pyridin-2-yl)cyclobutanecarbonitrile;
cis-1-(5-(2-methyl-2H-pyrazolo[3,4-b]pyridin-5-yl)[1,3]thiazolo[5,4-b]pyridin-2-yl)-3-(trifluoromethoxy)cyclobutanol;
trans-3-hydroxy-1-methyl-3-(5-(2-methyl-2H-pyrazolo[3,4-b]pyridin-5-yl)thieno[3,2-b]pyridin-2-yl)cyclobutanecarbonitrile;
cis-3-hydroxy-1-methyl-3-(5-(2-methyl-2H-pyrazolo [3,4-b]pyridin-5-yl)thieno[3,2-b]pyridin-2-yl)cyclobutanecarbonitrile;
5-(2-(2-methyl-2H-pyrazolo[3,4-b]pyridin-5-yl)thieno [2,3-d]pyrimidin-6-yl)spiro[2.3]hexan-5-ol;
5-(5-(2-methyl-2H-pyrazolo[3,4-b]pyridin-5-yl) [1,3]thiazolo[5,4-b]pyridin-2-yl)spiro[2.3]hexan-5-ol;
(R)-((1S)-2,2-difluorocyclobutyl)(6-(2-methyl-2H-pyrazolo[3,4-b]pyridin-5-yl)thieno[2,3-b]pyridin-2-yl)methanol;
(S)-((1R)-2,2-difluorocyclobutyl)(6-(2-methyl-2H-pyrazolo[3,4-b]pyridin-5-yl)thieno[2,3-b]pyridin-2-yl)methanol;
(R)-((1R)-2,2-difluorocyclobutyl)(6-(2-methyl-2H-pyrazolo[3,4-b]pyridin-5-yl)thieno[2,3-b]pyridin-2-yl)methanol;
(S)-((1S)-2,2-difluorocyclobutyl)(6-(2-methyl-2H-pyrazolo[3,4-b]pyridin-5-yl)thieno[2,3-b]pyridin-2-yl)methanol;
cis-3-(difluoromethyl)-1-(5-(2-methyl-2H-pyrazolo[3,4-b]pyridin-5-yl)[1,3]thiazolo[5,4-d]pyrimidin-2-yl)cyclobutanol;
cis-3-hydroxy-1-methyl-3-(6-(3-methyl-3H-[1,2,3]triazolo[4,5-b]pyridin-6-yl)thieno[2,3-b]pyridin-2-yl)cyclobutanecarbonitrile;
cis-1-(2-(3-methyl-3H-[1,2,3]triazolo[4,5-b]pyridin-6-yl)thieno[2,3-d]pyrimidin-6-yl)-3-(trifluoromethoxy)cyclobutanol;
cis-1-(5-(3-methyl-3H-[1,2,3]triazolo[4,5-b]pyridin-6-yl)[1,3]thiazolo[5,4-b]pyridin-2-yl)-3-(trifluoromethoxy)cyclobutanol;
trans-3-methoxy-1-(2-(2-methyl-2H-pyrazolo[3,4-b]pyridin-5-yl)thieno[2,3-d]pyrimidin-6-yl)-3-(trifluoromethyl)cyclobutanol;
cis-3-methoxy-1-(2-(2-methyl-2H-pyrazolo[3,4-b]pyridin-5-yl)thieno[2,3-d]pyrimidin-6-yl)-3-(trifluoromethyl)cyclobutanol;
trans-1,1-difluoro-5-(2-(2-methyl-2H-pyrazolo[3,4-b]pyridin-5-yl)thieno[2,3-d]pyrimidin-6-yl)spiro1[2.3]hexan-5-ol;
cis-1,1-difluoro-5-(2-(2-methyl-2H-pyrazolo[3,4-b]pyridin-5-yl)thieno[2,3-d]pyrimidin-6-yl)spiro1[2.3]hexan-5-ol;
trans-1,1-difluoro-5-(5-(2-methyl-2H-pyrazolo[3,4-b]pyridin-5-yl)[1,3]thiazolo[5,4-b]pyridin-2-yl)spiro1[2.3]hexan-5-ol;
cis-1,1-difluoro-5-(5-(2-methyl-2H-pyrazolo[3,4-b]pyridin-5-yl)[1,3]thiazolo[5,4-b]pyridin-2-yl)spiro1[2.3]hexan-5-ol;
6,6-difluoro-2-(2-(2-methyl-2H-pyrazolo[3,4-b]pyridin-5-yl)thieno[2,3-d]pyrimidin-6-yl)spiro[3.3]heptan-2-ol;
cis-1-(5-(2-methyl-2H-pyrazolo[3,4-b]pyridin-5-yl)thieno[3,2-b]pyridin-2-yl)-3-(trifluoromethoxy)cyclobutanol;
trans-3-methoxy-3-methyl-1-(2-(2-methyl-2H-pyrazolo[3,4-b]pyridin-5-yl)thieno[2,3-d]pyrimidin-6-yl)cyclobutanol;
cis-3-methoxy-3-methyl-1-(2-(2-methyl-2H-pyrazolo [3,4-b]pyridin-5-yl)thieno[2,3-d]pyrimidin-6-yl)cyclobutanol;
cis-3-(methoxymethyl)-1-(2-(2-methyl-2H-pyrazolo[3,4-b]pyridin-5-yl)thieno[2,3-d]pyrimidin-6-yl)cyclobutanol;
cis-1-(2-(1-methyl-1H-pyrazol-4-yl)thieno[2,3-d]pyrimidin-6-yl)-3-(trifluoromethyl)cyclobutanol;
(S)-(3-amino-6-(2-methyl-2H-pyrazolo[3,4-b]pyridin-5-yl)thieno[2,3-b]pyridin-2-yl)(3,3-difluorocyclobutyl)methanol;
(R)-(3-amino-6-(2-methyl-2H-pyrazolo [3,4-b]pyridin-5-yl)thieno [2,3-b]pyridin-2-yl)(3,3-difluorocyclobutyl)methanol;
trans-3-methoxy-3-methyl-1-(2-(3-methyl-3H-[1,2,3]triazolo[4,5-b]pyridin-6-yl)thieno[2,3-d]pyrimidin-6-yl)cyclobutanol;
cis-3-methoxy-3-methyl-1-(2-(3-methyl-3H-[1,2,3]triazolo[4,5-b]pyridin-6-yl)thieno[2,3-d]pyrimidin-6-yl)cyclobutanol;
cis-1-(2-(3-pyridinyl)thieno[2,3-d]pyrimidin-6-yl)-3-(trifluoromethyl)cyclobutanol;
cis-1-(2-(6-quinoxalinyl)thieno[2,3-d]pyrimidin-6-yl)-3-(trifluoromethyl)cyclobutanol;
cis-1-(2-(imidazo[1,2-a]pyrimidin-6-yl)thieno[2,3-d]pyrimidin-6-yl)-3-(trifluoromethyl)cyclobutanol;
trans-3-methoxy-3-methyl--(5-(2-methyl-2H-pyrazolo[3,4-b]pyridin-5-yl)[1,3]thiazolo[5,4-b]pyridin-2-yl)cyclobutanol;
cis-3-methoxy-3-methyl-1-(5-(2-methyl-2H-pyrazolo[3,4-b]pyridin-5-yl)[1,3]thiazolo[5,4-b]pyridin-2-yl)cyclobutanol;
trans-3-methoxy-3-methyl--(6-(3-methyl-3H-[1,2,3]triazolo[4,5-b]pyridin-6-yl)thieno[2,3-b]pyridin-2-yl)cyclobutanol;
cis-1-(2-(4-pyridazinyl)thieno[2,3-d]pyrimidin-6-yl)-3-(trifluoromethyl)cyclobutanol;
cis-3-methoxy-3-methyl-1-(5-(2-methyl-2H-pyrazolo[3,4-b]pyridin-5-yl)thieno[3,2-b]pyridin-2-yl)cyclobutanol;
3-(6-(cis-1-hydroxy-3-(trifluoromethyl)cyclobutyl)thieno[2,3-d]pyrimidin-2-yl)-5,6-dihydro-7H-pyrrolo [3,4-b]pyridin-7-one;
cis-1-(2-([1,2,4]triazolo[4,3-a]pyrimidin-6-yl)thieno[2,3-d]pyrimidin-6-yl)-3-(trifluoromethyl)cyclobutanol;
(R)-(3,3-difluorocyclobutyl)(2-(2-methyl-2H-pyrazolo[3,4-b]pyridin-5-yl)thieno[2,3-b]pyrazin-6-yl)methanol;
(S)-(3,3-difluorocyclobutyl)(2-(2-methyl-2H-pyrazolo[3,4-b]pyridin-5-yl)thieno[2,3-b]pyrazin-6-yl)methanol;
trans-3-methyl-1-(6-(2-methyl-2H-pyrazolo[3,4-b]pyridin-5-yl)thieno[2,3-b]pyridin-2-yl)-3-(trifluoromethyl)cyclobutanol;
cis-3-methyl-1-(6-(2-methyl-2H-pyrazolo[3,4-b]pyridin-5-yl)thieno[2,3-b]pyridin-2-yl)-3-(trifluoromethyl)cyclobutanol;
cis-1-(2-(2-methyl-2H-[1,2,3]triazolo[4,5-b]pyridin-6-yl)thieno[2,3-d]pyrimidin-6-yl)-3-(trifluoromethyl)cyclobutanol;
cis-1-(2-(1-methyl-1H-[1,2,3]triazolo[4,5-b]pyridin-6-yl)thieno[2,3-d]pyrimidin-6-yl)-3-(trifluoromethyl)cyclobutanol;
cis-1-(2-(1-methyl-1H-imidazo[4,5-b]pyridin-6-yl)thieno[2,3-d]pyrimidin-6-yl)-3-(trifluoromethyl)cyclobutanol;
cis-1-(2-(1,8-naphthyridin-3-yl)thieno[2,3-d]pyrimidin-6-yl)-3-(trifluoromethyl)cyclobutanol;
trans-3-(difluoromethyl)-1-(5-(2-methyl-2H-pyrazolo [3,4-b]pyridin-5-yl)thiazolo[5,4-b]pyridin-2-yl)cyclobutanol;
trans-3-methoxy-1-(2-(2-methyl-2H-pyrazolo[3,4-b]pyridin-5-yl)thieno[2,3-d]pyrimidin-6-yl)cyclobutan-i-ol; or
cis-1-(2-([1,2,4]triazolo[1,5-a]pyrimidin-6-yl)thieno[2,3-d]pyrimidin-6-yl)-3-(trifluoromethyl)cyclobutan-1-ol.

68-91. (canceled)

92. A pharmaceutical composition comprising the compound of claim 1, and a pharmaceutically acceptable excipient.

93-96. (canceled)

97. A method of treating intestinal, gastrointestinal, or bowel disorders in a subject in need thereof, the method comprising:
administering to the subject a therapeutically acceptable amount of a compound or salt according to claim 1.

98. A method of manufacturing the compound or salt of claim 1, the method comprising:
performing a Suzuki coupling between a compound of Formula (A-int-b) 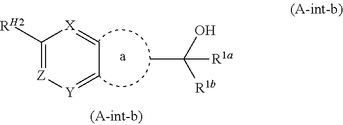
and a compound of Formula (R2-B): 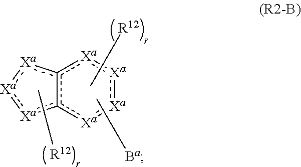
where
X^{H} is a —Br or —Cl;
B^{a} is a boronic acid group or a boronic ester group; [symbol]
indicates a double bond or single bond may be present;
each instance of X^{a} independently is nitrogen, oxygen, or carbon;
each instance of r, where present, is an integer selected from 0, 1, 2, 3, 4, or 5, wherein where two instances of r occur on the same R² group, the total of both r values combined does not exceed 5;
each instance of R¹², when present and attached to an N ring member, independently is C₁₋₆alkyl, C₁₋₆haloalkyl, C₁₋₆alkylene-OH, or C₁₋₆alkylene-O-C₁₋₆alkyl; and
each instance of R¹², when present and attached to a C ring member, independently is —OH, oxo, —N(R^{b})₂, C₁₋₆ alkyl, C₁₋₆ haloalkyl, C₁₋₆ alkoxy, C₁₋₆alkylene-OH, or C₁₋₆alkylene-O-C₁₋₆alkyl;
where each instance of R^{b}, where present, independently is —H or C₁₋₆ alkyl; or, alternatively, two R^{b} substituents together form a heterocyclyl having 3 to 6 ring members; and
wherein, when R^{b} is C₁₋₆ alkyl or, alternatively, two R^{b} substituents together form a heterocyclyl having 3 to 6 ring members, then R^{b} may be unsubstituted or substituted with one or more substituents, each of which independently is —F, —Cl, —OH, C₁₋₃ alkoxy.

99. A compound or salt of claim 1, made by a method comprising:
performing a Suzuki coupling between a compound of Formula (A-int-b) 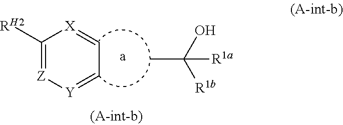
and a compound of Formula (R2-B): 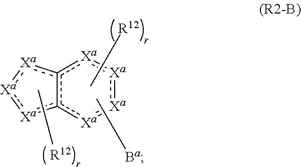
where
X^{H} is a —Br or —Cl;
B^{a} is a boronic acid group or a boronic ester group; [symbol]
indicates a double bond or single bond may be present;
each instance of X^{a} independently is nitrogen, oxygen, or carbon;
each instance of r, where present, is an integer selected from 0, 1, 2, 3, 4, or 5, wherein where two instances of r occur on the same R² group, the total of both r values combined does not exceed 5;
each instance of R¹², when present and attached to an N ring member, independently is C₁₋₆alkyl, C₁₋₆haloalkyl, C₁₋₆alkylene-OH, or C₁₋₆alkylene-O-C₁₋₆alkyl; and
each instance of R¹², when present and attached to a C ring member, independently is —OH, oxo, —N(R^{b})₂, C₁₋₆ alkyl, C₁₋₆ haloalkyl, C₁₋₆ alkoxy, C₁₋₆alkylene-OH, or C₁₋₆alkylene-O-C₁₋₆ alkyl;
where each instance of R^{b}, where present, independently is —H or C₁₋₆ alkyl; or, alternatively, two R^{b} substituents together form a heterocyclyl having 3 to 6 ring members; and
wherein, when R^{b} is C₁₋₆ alkyl or, alternatively, two R^{b} substituents together form a heterocyclyl having 3 to 6 ring members, then R^{b} may be unsubstituted or substituted with one or more substituents, each of which independently is —F, —Cl, —OH, C₁₋₃ alkoxy.

100-101. (canceled)
